(19) United States
(12) Patent Application Publication
(10) Pub. No.: US 2004/0038223 A1
(43) Pub. Date: Feb. 26, 2004

(54) Novel proteins and nucleic acids encoding same

(76) Inventors: Glennda Smithson, Guilford, CT (US); Isabelle Millet, Milford, CT (US); John A. Peyman, New Haven, CT (US); Ramesh Kekuda, Norwalk, CT (US); Jingfang Ju, Orange, CT (US); Li Li, Branford, CT (US); Xiaojia (Sasha) Guo, Branford, CT (US); Meera Patturajan, Branford, CT (US); Kimberly A. Spytek, New Haven, CT (US); Shlomit R. Edinger, New Haven, CT (US); Karen Ellerman, Branford, CT (US); Uriel M. Malyankar, Branford, CT (US); Tatiana Ort, Milford, CT (US); Linda Gorman, Branford, CT (US); Bryan D. Zerhusen, Branford, CT (US); David W. Anderson, Branford, CT (US); Mei Zhong, Branford, CT (US); Elina Catterton, Madison, CT (US); Weizhen Ji, Branford, CT (US); Charles E. Miller, Guilford, CT (US); Luca Rastelli, Guilford, CT (US); David J. Stone, Guilford, CT (US); Carol E. A. Pena, New Haven, CT (US); Suresh G. Shenoy, Branford, CT (US); Richard A. Shimkets, Guilford, CT (US); Mark E. Rothenberg, Clinton, CT (US); Martin D. Leach, Madison, CT (US); Michele L. Agee, Wallingford, CT (US); Constance Berghs, New Haven, CT (US); Vincent A. DiPippo, East Haven, CT (US); Andrew Eisen, Rockville, MD (US); Esha A. Gangolli, Madison, CT (US); Daniel K. Rieger, Branford, CT (US); Steven K. Spaderna, Berlin, CT (US)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/262,511

(22) Filed: Oct. 1, 2002

Related U.S. Application Data
(60) Provisional application No. 60/326,483, filed on Oct. 2, 2001.
(60) Provisional application No. 60/373,815, filed on Apr. 19, 2002.
(60) Provisional application No. 60/327,917, filed on Oct. 9, 2001.
(60) Provisional application No. 60/381,642, filed on May 17, 2002.
(60) Provisional application No. 60/328,029, filed on Oct. 9, 2001.
(60) Provisional application No. 60/381,038, filed on May 16, 2002.
(60) Provisional application No. 60/328,056, filed on Oct. 9, 2001.
(60) Provisional application No. 60/373,260, filed on Apr. 17, 2002.
(60) Provisional application No. 60/373,826, filed on Apr. 19, 2002.
(60) Provisional application No. 60/327,435, filed on Oct. 5, 2001.
(60) Provisional application No. 60/327,449, filed on Oct. 5, 2001.
(60) Provisional application No. 60/373,884, filed on Apr. 19, 2002.
(60) Provisional application No. 60/328,044, filed on Oct. 9, 2001.
(60) Provisional application No. 60/374,977, filed on Apr. 22, 2002.
(60) Provisional application No. 60/381,042, filed on May 16, 2002.
(60) Provisional application No. 60/328,849, filed on Oct. 12, 2001.
(60) Provisional application No. 60/329,414, filed on Oct. 15, 2001.
(60) Provisional application No. 60/330,142, filed on Oct. 17, 2001.
(60) Provisional application No. 60/330,309, filed on Oct. 18, 2001.
(60) Provisional application No. 60/341,058, filed on Oct. 22, 2001.
(60) Provisional application No. 60/343,629, filed on Oct. 24, 2001.
(60) Provisional application No. 60/383,831, filed on May 29, 2002.
(60) Provisional application No. 60/339,266, filed on Oct. 24, 2001.
(60) Provisional application No. 60/391,335, filed on Jun. 25, 2002.
(60) Provisional application No. 60/349,575, filed on Oct. 29, 2001.
(60) Provisional application No. 60/383,656, filed on May 28, 2002.
(60) Provisional application No. 60/373,817, filed on Apr. 19, 2002.
(60) Provisional application No. 60/381,037, filed on May 16, 2002.
(60) Provisional application No. 60/346,357, filed on Nov. 1, 2001.

(51) Int. Cl. C12Q 1/68; G01N 33/53; C07H 21/04; C12N 9/00; C07K 14/47; C12P 21/02; C12N 5/06

(52) U.S. Cl. 435/6; 435/7.1; 435/69.1; 435/183; 435/320.1; 435/325; 530/350; 514/12; 536/23.2

(57) ABSTRACT

The present invention provides novel isolated polynucleotides and small molecule target polypeptides encoded by the polynucleotides. Antibodies that immunospecifically bind to a novel small molecule target polypeptide or any derivative, variant, mutant or fragment of that polypeptide, polynucleotide or antibody are disclosed, as are methods in which the small molecule target polypeptide, polynucleotide and antibody are utilized in the detection and treatment of a broad range of pathological states. More specifically, the present invention discloses methods of using recombinantly expressed and/or endogenously expressed proteins in various screening procedures for the purpose of identifying therapeutic antibodies and therapeutic small molecules associated with diseases. The invention further discloses therapeutic, diagnostic and research methods for diagnosis, treatment, and prevention of disorders involving any one of these novel human nucleic acids and proteins.

### RELATED APPLICATIONS

This application claims priority to provisional patent applications U.S. Ser. No. 60/326,483, filed Oct. 2, 2001; U.S. Ser. No. 60/373,815, filed Apr. 19, 2002; U.S. Ser. No. 60/327,917, filed Oct. 9, 2001; U.S. Ser. No. 60/381,642, filed May 17, 2002; U.S. Ser. No. 60/328,029, filed Oct. 9, 2001; U.S. Ser. No. 60/381,038, filed May 16, 2002; U.S. Ser. No. 60/328,056, filed Oct. 9, 2001; U.S. Ser. No. 60/373,260, filed Apr. 17, 2002; U.S. Ser. No. 60/373,826, filed Apr. 19, 2002; U.S. Ser. No. 60/327,435, filed Oct. 5, 2001; U.S. Ser. No. 60/327,449, filed Oct. 5, 2001; U.S. Ser. No. 60/373,884, filed Apr. 19, 2002; U.S. Ser. No. 60/328,044, filed Oct. 9, 2001; U.S. Ser. No. 60/374,977, filed Apr. 22, 2002; U.S. Ser. No. 60/381,042, filed May 16, 2002; U.S. Ser. No. 60/328,849, filed Oct. 12, 2001; U.S. Ser. No. 60/329,414, filed Oct. 15, 2001; U.S. Ser. No. 60/330,142, filed Oct. 17, 2001; U.S. Ser. No. 60/330,309, filed Oct. 18, 2001; U.S. Ser. No. 60/341,058, filed Oct. 22, 2001; U.S. Ser. No. 60/343,629, filed Oct. 24, 2001; U.S. Ser. No. 60/383,831, filed May 29, 2002; U.S. Ser. No. 60/339,266, filed Oct. 24, 2001; U.S. Ser. No. 60/391,335, filed Jun. 25, 2002; U.S. Ser. No. 60/349,575, filed Oct. 29, 2001; U.S. Ser. No. 60/383,656, filed May 28, 2002; U.S. Ser. No. 60/373,817, filed Apr. 19, 2002; U.S. Ser. No. 60/381,037, filed May 16, 2002; U.S. Ser. No. 60/346,357, filed Nov. 1, 2001; each of which is incorporated herein by reference in its entirety.

### FIELD OF THE INVENTION

The present invention relates to novel polypeptides that are targets of small molecule drugs and that have properties related to stimulation of biochemical or physiological responses in a cell, a tissue, an organ or an organism. More particularly, the novel polypeptides are gene products of novel genes, or are specified biologically active fragments or derivatives thereof. Methods of use encompass diagnostic and prognostic assay procedures as well as methods of treating diverse pathological conditions.

### BACKGROUND

Eukaryotic cells are characterized by biochemical and physiological processes which under normal conditions are exquisitely balanced to achieve the preservation and propagation of the cells. When such cells are components of multicellular organisms such as vertebrates, or more particularly organisms such as mammals, the regulation of the biochemical and physiological processes involves intricate signaling pathways. Frequently, such signaling pathways involve extracellular signaling proteins, cellular receptors that bind the signaling proteins and signal transducing components located within the cells.

Signaling proteins may be classified as endocrine effectors, paracrine effectors or autocrine effectors. Endocrine effectors are signaling molecules secreted by a given organ into the circulatory system, which are then transported to a distant target organ or tissue. The target cells include the receptors for the endocrine effector, and when the endocrine effector binds, a signaling cascade is induced. Paracrine effectors involve secreting cells and receptor cells in close proximity to each other, for example two different classes of cells in the same tissue or organ. One class of cells secretes the paracrine effector, which then reaches the second class of cells, for example by diffusion through the extracellular fluid. The second class of cells contains the receptors for the paracrine effector; binding of the effector results in induction of the signaling cascade that elicits the corresponding biochemical or physiological effect. Autocrine effectors are highly analogous to paracrine effectors, except that the same cell type that secretes the autocrine effector also contains the receptor. Thus the autocrine effector binds to receptors on the same cell, or on identical neighboring cells. The binding process then elicits the characteristic biochemical or physiological effect.

Signaling processes may elicit a variety of effects on cells and tissues including by way of nonlimiting example induction of cell or tissue proliferation, suppression of growth or proliferation, induction of differentiation or maturation of a cell or tissue, and suppression of differentiation or maturation of a cell or tissue.

Many pathological conditions involve dysregulation of expression of important effector proteins. In certain classes of pathologies the dysregulation is manifested as diminished or suppressed level of synthesis and secretion of protein effectors. In other classes of pathologies the dysregulation is manifested as increased or up-regulated level of synthesis and secretion of protein effectors. In a clinical setting a subject may be suspected of suffering from a condition brought on by altered or mis-regulated levels of a protein effector of interest. Therefore there is a need to assay for the level of the protein effector of interest in a biological sample from such a subject, and to compare the level with that characteristic of a nonpathological condition. There also is a need to provide the protein effector as a product of manufacture. Administration of the effector to a subject in need thereof is useful in treatment of the pathological condition. Accordingly, there is a need for a method of treatment of a pathological condition brought on by a diminished or suppressed levels of the protein effector of interest. In addition, there is a need for a method of treatment of a pathological condition brought on by a increased or up-regulated levels of the protein effector of interest.

Small molecule targets have been implicated in various disease states or pathologies. These targets may be proteins, and particularly enzymatic proteins, which are acted upon by small molecule drugs for the purpose of altering target function and achieving a desired result. Cellular, animal and clinical studies can be performed to elucidate the genetic contribution to the etiology and pathogenesis of conditions in which small molecule targets are implicated in a variety of physiologic, pharmacologic or native states. These studies utilize the core technologies at CuraGen Corporation to look at differential gene expression, protein-protein interactions, large-scale sequencing of expressed genes and the association of genetic variations such as, but not limited to, single nucleotide polymorphisms (SNPs) or splice variants in and between biological samples from experimental and control groups. The goal of such studies is to identify potential avenues for therapeutic intervention in order to prevent, treat the consequences or cure the conditions.

In order to treat diseases, pathologies and other abnormal states or conditions in which a mammalian organism has been diagnosed as being, or as being at risk for becoming, other than in a normal state or condition, it is important to identify new therapeutic agents. Such a procedure includes at least the steps of identifying a target component within an affected tissue or organ, and identifying a candidate therapeutic agent that modulates the functional attributes of the target. The target component may be any biological macromolecule implicated in the disease or pathology. Commonly the target is a polypeptide or protein with specific functional attributes. Other classes of macromolecule may be a nucleic acid, a polysaccharide, a lipid such as a complex lipid or a glycolipid; in addition a target may be a sub-cellular structure or extra-cellular structure that is comprised of more than one of these classes of macromolecule. Once such a target has been identified, it may be employed in a screening assay in order to identify favorable candidate therapeutic agents from among a large population of substances or compounds.

In many cases the objective of such screening assays is to identify small molecule candidates; this is commonly approached by the use of combinatorial methodologies to develop the population of substances to be tested. The implementation of high throughput screening methodologies is advantageous when working with large, combinatorial libraries of compounds.

### SUMMARY OF THE INVENTION

The invention includes nucleic acid sequences and the novel polypeptides they encode. The novel nucleic acids and polypeptides are referred to herein as NOVX, or NOV1, NOV2, NOV3, etc., nucleic acids and polypeptides. These nucleic acids and polypeptides, as well as derivatives, homologs, analogs and fragments thereof, will hereinafter be collectively designated as “NOVX” nucleic acid, which represents the nucleotide sequence selected from the group consisting of SEQ ID NO: 2n−1, wherein n is an integer between 1 and 124, or polypeptide sequences, which represents the group consisting of SEQ ID NO: 2n, wherein n is an integer between 1 and 124.

In one aspect, the invention provides an isolated polypeptide comprising a mature form of a NOVX amino acid. One example is a variant of a mature form of a NOVX amino acid sequence, wherein any amino acid in the mature form is changed to a different amino acid, provided that no more than 15% of the amino acid residues in the sequence of the mature form are so changed. The amino acid can be, for example, a NOVX amino acid sequence or a variant of a NOVX amino acid sequence, wherein any amino acid specified in the chosen sequence is changed to a different amino acid, provided that no more than 15% of the amino acid residues in the sequence are so changed. The invention also includes fragments of any of these. In another aspect, the invention also includes an isolated nucleic acid that encodes a NOVX polypeptide, or a fragment, homolog, analog or derivative thereof.

Also included in the invention is a NOVX polypeptide that is a naturally occurring allelic variant of a NOVX sequence. In one embodiment, the allelic variant includes an amino acid sequence that is the translation of a nucleic acid sequence differing by a single nucleotide from a NOVX nucleic acid sequence. In another embodiment, the NOVX polypeptide is a variant polypeptide described therein, wherein any amino acid specified in the chosen sequence is changed to provide a conservative substitution. In one embodiment, the invention discloses a method for determining the presence or amount of the NOVX polypeptide in a sample. The method involves the steps of: providing a sample; introducing the sample to an antibody that binds immunospecifically to the polypeptide; and determining the presence or amount of antibody bound to the NOVX polypeptide, thereby determining the presence or amount of the NOVX polypeptide in the sample. In another embodiment, the invention provides a method for determining the presence of or predisposition to a disease associated with altered levels of a NOVX polypeptide in a mammalian subject. This method involves the steps of: measuring the level of expression of the polypeptide in a sample from the first mammalian subject; and comparing the amount of the polypeptide in the sample of the first step to the amount of the polypeptide present in a control sample from a second mammalian subject known not to have, or not to be predisposed to, the disease, wherein an alteration in the expression level of the polypeptide in the first subject as compared to the control sample indicates the presence of or predisposition to the disease.

In a further embodiment, the invention includes a method of identifying an agent that binds to a NOVX polypeptide. This method involves the steps of: introducing the polypeptide to the agent; and determining whether the agent binds to the polypeptide. In various embodiments, the agent is a cellular receptor or a downstream effector.

In another aspect, the invention provides a method for identifying a potential therapeutic agent for use in treatment of a pathology, wherein the pathology is related to aberrant expression or aberrant physiological interactions of a NOVX polypeptide. The method involves the steps of: providing a cell expressing the NOVX polypeptide and having a property or function ascribable to the polypeptide; contacting the cell with a composition comprising a candidate substance; and determining whether the substance alters the property or function ascribable to the polypeptide; whereby, if an alteration observed in the presence of the substance is not observed when the cell is contacted with a composition devoid of the substance, the substance is identified as a potential therapeutic agent. In another aspect, the invention describes a method for screening for a modulator of activity or of latency or predisposition to a pathology associated with the NOVX polypeptide. This method involves the following steps: administering a test compound to a test animal at increased risk for a pathology associated with the NOVX polypeptide, wherein the test animal recombinantly expresses the NOVX polypeptide. This method involves the steps of measuring the activity of the NOVX polypeptide in the test animal after administering the compound of step; and comparing the activity of the protein in the test animal with the activity of the NOVX polypeptide in a control animal not administered the polypeptide, wherein a change in the activity of the NOVX polypeptide in the test animal relative to the control animal indicates the test compound is a modulator of latency of, or predisposition to, a pathology associated with the NOVX polypeptide. In one embodiment, the test animal is a recombinant test animal that expresses a test protein transgene or expresses the transgene under the control of a promoter at an increased level relative to a wild-type test animal, and wherein the promoter is not the native gene promoter of the transgene. In another aspect, the invention includes a method for modulating the activity of the NOVX polypeptide, the method comprising introducing a cell sample expressing the NOVX polypeptide with a compound that binds to the polypeptide in an amount sufficient to modulate the activity of the polypeptide.

The invention also includes an isolated nucleic acid that encodes a NOVX polypeptide, or a fragment, homolog, analog or derivative thereof. In a preferred embodiment, the nucleic acid molecule comprises the nucleotide sequence of a naturally occurring allelic nucleic acid variant. In another embodiment, the nucleic acid encodes a variant polypeptide, wherein the variant polypeptide has the polypeptide sequence of a naturally occurring polypeptide variant. In another embodiment, the nucleic acid molecule differs by a single nucleotide from a NOVX nucleic acid sequence. In one embodiment, the NOVX nucleic acid molecule hybridizes under stringent conditions to the nucleotide sequence selected from the group consisting of SEQ ID NO: 2n−1, wherein n is an integer between 1 and 124, or a complement of the nucleotide sequence. In another aspect, the invention provides a vector or a cell expressing a NOVX nucleotide sequence.

In one embodiment, the invention discloses a method for modulating the activity of a NOVX polypeptide. The method includes the steps of: introducing a cell sample expressing the NOVX polypeptide with a compound that binds to the polypeptide in an amount sufficient to modulate the activity of the polypeptide. In another embodiment, the invention includes an isolated NOVX nucleic acid molecule comprising a nucleic acid sequence encoding a polypeptide comprising a NOVX amino acid sequence or a variant of a mature form of the NOVX amino acid sequence, wherein any amino acid in the mature form of the chosen sequence is changed to a different amino acid, provided that no more than 15% of the amino acid residues in the sequence of the mature form are so changed. In another embodiment, the invention includes an amino acid sequence that is a variant of the NOVX amino acid sequence, in which any amino acid specified in the chosen sequence is changed to a different amino acid, provided that no more than 15% of the amino acid residues in the sequence are so changed.

In one embodiment, the invention discloses a NOVX nucleic acid fragment encoding at least a portion of a NOVX polypeptide or any variant of the polypeptide, wherein any amino acid of the chosen sequence is changed to a different amino acid, provided that no more than 10% of the amino acid residues in the sequence are so changed. In another embodiment, the invention includes the complement of any of the NOVX nucleic acid molecules or a naturally occurring allelic nucleic acid variant. In another embodiment, the invention discloses a NOVX nucleic acid molecule that encodes a variant polypeptide, wherein the variant polypeptide has the polypeptide sequence of a naturally occurring polypeptide variant. In another embodiment, the invention discloses a NOVX nucleic acid, wherein the nucleic acid molecule differs by a single nucleotide from a NOVX nucleic acid sequence.

In another aspect, the invention includes a NOVX nucleic acid, wherein one or more nucleotides in the NOVX nucleotide sequence is changed to a different nucleotide provided that no more than 15% of the nucleotides are so changed. In one embodiment, the invention discloses a nucleic acid fragment of the NOVX nucleotide sequence and a nucleic acid fragment wherein one or more nucleotides in the NOVX nucleotide sequence is changed from that selected from the group consisting of the chosen sequence to a different nucleotide provided that no more than 15% of the nucleotides are so changed. In another embodiment, the invention includes a nucleic acid molecule wherein the nucleic acid molecule hybridizes under stringent conditions to a NOVX nucleotide sequence or a complement of the NOVX nucleotide sequence. In one embodiment, the invention includes a nucleic acid molecule, wherein the sequence is changed such that no more than 15% of the nucleotides in the coding sequence differ from the NOVX nucleotide sequence or a fragment thereof.

In a further aspect, the invention includes a method for determining the presence or amount of the NOVX nucleic acid in a sample. The method involves the steps of: providing the sample; introducing the sample to a probe that binds to the nucleic acid molecule; and determining the presence or amount of the probe bound to the NOVX nucleic acid molecule, thereby determining the presence or amount of the NOVX nucleic acid molecule in the sample. In one embodiment, the presence or amount of the nucleic acid molecule is used as a marker for cell or tissue type.

In another aspect, the invention discloses a method for determining the presence of or predisposition to a disease associated with altered levels of the NOVX nucleic acid molecule of in a first mammalian subject. The method involves the steps of: measuring the amount of NOVX nucleic acid in a sample from the first mammalian subject; and comparing the amount of the nucleic acid in the sample of step (a) to the amount of NOVX nucleic acid present in a control sample from a second mammalian subject known not to have or not be predisposed to, the disease; wherein an alteration in the level of the nucleic acid in the first subject as compared to the control sample indicates the presence of or predisposition to the disease.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The present invention provides novel nucleotides and polypeptides encoded thereby. Included in the invention are the novel nucleic acid sequences, their encoded polypeptides, antibodies, and other related compounds. The sequences are collectively referred to herein as “NOVX nucleic acids” or “NOVX polynucleotides” and the corresponding encoded polypeptides are referred to as “NOVX polypeptides” or “NOVX proteins.” Unless indicated otherwise, “NOVX” is meant to refer to any of the novel sequences disclosed herein. Table A provides a summary of the NOVX nucleic acids and their encoded polypeptides.

TABLE A
Sequences and Corresponding SEQ ID Numbers

| | | SEQ ID | SEQ ID | |
|---|---|---|---|---|
| | | NO | NO | |
| NOVX | Internal | (nucleic | (amino | |
| Assignment | Identification | acid) | acid) | Homology |
| 1a | CG106764-01 | 1 | 2 | Citron Kinase |
| 1b | 268667493 | 3 | 4 | RHO/RAC-Interacting Citron Kinase |
| 1c | 268667539 | 5 | 6 | RHO/RAC-Interacting Citron Kinase |
| 1d | 268667543 | 7 | 8 | RHO/RAC-Interacting Citron Kinase |
| 1e | 268667555 | 9 | 10 | RHO/RAC-Interacting Citron Kinase |
| 1f | 268667574 | 11 | 12 | RHO/RAC-Interacting Citron Kinase |
| 1g | CG106764-02 | 13 | 14 | RHO/RAC-Interacting Citron Kinase |
| 2a | CG117662-01 | 15 | 16 | Renal Renin Precursor |
| 2b | CG117662-02 | 17 | 18 | Renal Renin Precursor |
| 3a | CG118051-03 | 19 | 20 | Aldehyde Dehydrogenase |
| 3b | CG118051-02 | 21 | 22 | Aldehyde Dehydrogenase |
| 3c | CG118051-03 | 23 | 24 | Aldehyde Dehydrogenase |
| 4a | CG120277-01 | 25 | 26 | Aldehyde Dehydrogenase-3 |
| 4b | CG120277-02 | 27 | 28 | Aldehyde Dehydrogenase-3 |
| 5a | CG140468-01 | 29 | 30 | Serine/Threonine-Protein Kinase PAK 1 |
| 5b | CG140468-02 | 31 | 32 | Serine/Threonine-Protein Kinase PAK 1 |
| 6a | CG142182-01 | 33 | 34 | Ubiquitin Carboxyl-terminal Hydrolase |
| | | | | 15 |
| 7a | CG142564-01 | 35 | 36 | Carnitine O-Palmitoyltransferase I |
| 8a | CG142797-01 | 37 | 38 | Cathepsin L |
| 9a | CG143216-01 | 39 | 40 | Laminin Gamma 3 Chain Precursor |
| 10a | CG143787-01 | 41 | 42 | Disintegrin Protease |
| 10b | 278889162 | 43 | 44 | Disintegrin Protease |
| 10c | 278689868 | 45 | 46 | Disintegrin Protease |
| 11a | CG144112-01 | 47 | 48 | NEUROPSIN PRECURSOR like homo |
| | | | | sapiens |
| 11b | CG144112-04 | 49 | 50 | Neuropsin Precursor |
| 11c | 255501898 | 51 | 52 | Neuropsin Precursor |
| 11d | 255612524 | 53 | 54 | Neuropsin Precursor |
| 11e | 255612566 | 55 | 56 | Neuropsin Precursor |
| 11f | 306434072 | 57 | 58 | Neuropsin Precursor |
| 11g | CG144112-02 | 59 | 60 | Neuropsin Precursor |
| 11h | CG144112-03 | 61 | 62 | Neuropsin Precursor |
| 12a | CG144497-01 | 63 | 64 | Adenylosuccinate Synthetase Muscle |
| | | | | Isozyme |
| 13a | CG144686-01 | 65 | 66 | Mast Cell Carboxypeptidase A Precursor |
| 13b | 278690008 | 67 | 68 | Mast Cell Carboxypeptidase A Precursor |
| 13c | 278690035 | 69 | 70 | Mast Cell Carboxypeptidase A Precursor |
| 13d | CG144686-02 | 71 | 72 | Mast Cell Carboxypeptidase A Precursor |
| 14a | CG144906-01 | 73 | 74 | Testisin Precursor |
| 14b | CG144906-02 | 75 | 76 | Testisin Precursor |
| 15a | CG144997-01 | 77 | 78 | RNase H I |
| 15b | 278693648 | 79 | 80 | RNase H I |
| 15c | 278480974 | 81 | 82 | RNase H I |
| 15d | 278498047 | 83 | 84 | RNase H I |
| 15e | CG144997-02 | 85 | 86 | RNaseHI |
| 16a | CG145494-01 | 87 | 88 | PRESTIN |
| 17a | CG145722-01 | 89 | 90 | WEE1 |
| 18a | CG145754-01 | 91 | 92 | Kallikrein 7 Precursor |
| 18b | CG145754-03 | 93 | 94 | Kallikrein 7 Precursor |
| 18c | CG145754-02 | 95 | 96 | Kallikrein 7 Precursor |
| 18d | 252718128 | 97 | 98 | Kallikrein |
| 18e | 252718152 | 99 | 100 | Kallikrein |
| 18f | 247856668 | 101 | 102 | Kallikrein 7 Precursor |
| 18g | 247856705 | 103 | 104 | Kallikrein 7 Precursor |
| 19a | CG146279-01 | 105 | 106 | Novel Potassium Channel Subfamily K |
| | | | | Member 10 (TREK-2) |
| 20a | CG146374-01 | 107 | 108 | Glycogen Branching Enzyme |
| 21a | CG146403-01 | 109 | 110 | Diacylglycerol Acyltransferase 2 |
| 22a | CG146513-01 | 111 | 112 | Diacyiglycerol Acyltransferase 2 |
| 23a | CG146522-01 | 113 | 114 | Diacyiglycerol Acyltransferase 2 |
| 24a | CG146531-01 | 115 | 116 | Diacylglycerol Acyltransferase 2 |
| 25a | CG147274-01 | 117 | 118 | Protease |
| 26a | CG147351-01 | 119 | 120 | Testis-Development Related NYD-SP27 |
| 27a | CG147419-01 | 121 | 122 | Glutamine: Fructose-6-Phosphate |
| | | | | Amidotransferase 1 Muscle Isoform |
| 28a | CG148102-01 | 123 | 124 | Carnitine 0-Palmitoyltransferase |
| 28b | CG148102-02 | 125 | 126 | Carnitine 0-Palmitoyltransferase |
| 29a | CG148431-01 | 127 | 128 | Class II Aminotransferase |
| 29b | CG148431-02 | 129 | 130 | Class II Aminotransferase |
| 30a | CG148888-01 | 131 | 132 | GALNAC 4-Sulfotransferase |
| 31a | CG149008-01 | 133 | 134 | Sodium/Hydrogen Exchanger |
| 32a | CG149350-01 | 135 | 136 | Vacuolar ATP Synthase Subunit F |
| 32b | CG149350-02 | 137 | 138 | Vacuolar ATP Synthase Subunit F |
| 33a | CG149463-01 | 139 | 140 | Serine/Threonine-Protein Kinase SGK |
| 34a | CG149536-01 | 141 | 142 | Protein-Tyrosine Phosphatase, |
| | | | | Non-Receptor Type 2 |
| 35a | CG149964-01 | 143 | 144 | Brain Mitochondrial Carrier Protein-1 |
| 35b | 309326356 | 145 | 146 | Brain Mitochondrial Carrier Protein-1 |
| 35c | 309326444 | 147 | 148 | Brain Mitochondrial Carrier Protein-1 |
| 35d | 309326473 | 149 | 150 | Brain Mitochondrial Carrier Protein-1 |
| 35e | CG149964-02 | 151 | 152 | Brain Mitochondrial Carrier Protein-1 |
| 36a | CG150306-01 | 153 | 154 | Dual Specificity Protein Phosphatase 5 |
| 37a | CG150510-01 | 155 | 156 | Human Alpha-2,3-Sialyltransferase |
| 38a | CG150704-01 | 157 | 158 | Testis ecto-ADP-Ribosyltransferase |
| | | | | Precursor |
| 39a | CG150799-01 | 159 | 160 | MASS1 |
| 39b | CG150799-02 | 161 | 162 | MASS1 |
| 39c | CG150799-03 | 163 | 164 | MASS1 |
| 39d | CG150799-01 | 165 | 166 | MASS1 |
| 40a | CG1S1O14-01 | 167 | 168 | Metabotropic Glutamate Receptor 3 |
| 40b | CG151014-02 | 169 | 170 | Metabotropic Glutamate Receptor 3 |
| 40c | CG151014-03 | 171 | 172 | Metabotropic Glutamate Receptor 3 |
| 41a | CG151297-01 | 173 | 174 | Calmodulin-Dependent |
| | | | | Phosphodiesterase |
| 41b | CG151297-02 | 175 | 176 | Calmodulin-Dependent |
| | | | | Phosphodiesterase |
| 42a | CG151822-01 | 177 | 178 | Prenylcysteine Carboxyl |
| | | | | Methyltransferase |
| 42b | CG151822-02 | 179 | 180 | Prenylcysteine Carboxyl |
| | | | | Methyltransferase |
| 43a | CG152256-01 | 181 | 182 | Phosphatidylserine Synthase |
| 44a | CG171804-01 | 183 | 184 | N-Acetylgalactosaminide Alpha 2, |
| | | | | 6-Sialyltransferase |
| 45a | CG171841-01 | 185 | 186 | Iron-Containing Alcohol Dehydrogenase |
| 46a | CG173017-01 | 187 | 188 | Retinoic Acid Receptor RXR-Beta |
| 47a | CG173347-01 | 189 | 190 | Serum Paraoxonase/Arylesterase 3 |
| 48a | CG56234-01 | 191 | 192 | Phosphoenolpyruvate Carboxykinase 2 |
| | | | | (PCK2) |
| 48b | CG56234-02 | 193 | 194 | Phosphoenolpyruvate Carboxykinase 2 |
| | | | | (PCK2) |
| 49a | CG56836-01 | 195 | 196 | Cathepsin B |
| 49b | CG56836-02 | 197 | 198 | Cathepsin B |
| 49c | CG56836-03 | 199 | 200 | Cathepsin B |
| 49d | CG56836-04 | 201 | 202 | Cathepsin B |
| 49e | 247856403 | 203 | 204 | Cathepsin B |
| 49f | 247856434 | 205 | 206 | Cathepsin B |
| 49g | 247856497 | 207 | 208 | Cathepsin B |
| 49h | 247856493 | 209 | 210 | Cathepsin B |
| 49i | 247856574 | 211 | 212 | Cathepsin B |
| 49j | 247856545 | 213 | 214 | Cathepsin B |
| 49k | 275480714 | 215 | 216 | Cathepsin B |
| 50a | CG57284-01 | 217 | 218 | RAS-Related Protein RAB-5C |
| 50b | CG57284-03 | 219 | 220 | RAS-Related Protein RAB-5C |
| 50c | CG57284-02 | 221 | 222 | RAS-Related Protein RAB-5C |
| 51a | CG57308-01 | 223 | 224 | Sulfonylurea Receptor 1 |
| 51b | CG57308-02 | 225 | 226 | Sulfonylurea Receptor 1 |
| 52a | CG93659-01 | 227 | 228 | Mitogen-Activated Protein Kinase |
| | | | | Kinase Kinase 8 |
| 52b | CG93659-03 | 229 | 230 | Mitogen-Activated Protein Kinase |
| | | | | Kinase Kinase 8 |
| 52c | CG93659-02 | 231 | 232 | Mitogen-Activated Protein Kinase |
| | | | | Kinase Kinase 8 |
| 53a | CG94521-01 | 233 | 234 | Cytoplasmic Glycerol-3-Phosphate |
| | | | | Dehydrogenase [NAD+] |
| 53b | CG94521-03 | 235 | 236 | Cytoplasmic Glycerol-3-Phosphate |
| | | | | Dehydrogenase [NAD+] |
| 53c | CG94521-02 | 237 | 238 | Cytoplasmic Glycerol-3-Phosphate |
| | | | | Dehydrogenase [NAD+] |
| 54a | CG96613-01 | 239 | 240 | Pyruvate Dehydrogenase Kinase (PDK1) |
| 54b | CG96613-03 | 241 | 242 | Pyruvate Dehydrogenase Kinase (PDK1) |
| 54c | CG96613-02 | 243 | 244 | Pyruvate Dehydrogenase Kinase (PDK1) |
| 55a | CG96736-01 | 245 | 246 | Neutral Amino Acid Transporter B |
| 55b | CG96736-02 | 247 | 248 | Neutral Amino Acid Transporter B |

Table A indicates the homology of NOVX polypeptides to known protein families. Thus, the nucleic acids and polypeptides, antibodies and related compounds according to the invention corresponding to a NOVX as identified in column 1 of Table A will be useful in therapeutic and diagnostic applications implicated in, for example, pathologies and disorders associated with the known protein families identified in column 5 of Table A.

Pathologies, diseases, disorders and condition and the like that are associated with NOVX sequences include, but are not limited to: e.g., cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, metabolic disturbances associated with obesity, transplantation, adrenoleukodystrophy, congenital adrenal hyperplasia, prostate cancer, diabetes, metabolic disorders, neoplasm; adenocarcinoma, lymphoma, uterus cancer, fertility, hemophilia, hypercoagulation, idiopathic thrombocytopenic purpura, immunodeficiencies, graft versus host disease, AIDS, bronchial asthma, Crohn's disease; multiple sclerosis, treatment of Albright Hereditary Ostoeodystrophy, infectious disease, anorexia, cancer-associated cachexia, cancer, neurodegenerative disorders, Alzheimer's Disease, Parkinson's Disorder, immune disorders, hematopoietic disorders, and the various dyslipidemias, the metabolic syndrome X and wasting disorders associated with chronic diseases and various cancers, as well as conditions such as transplantation and fertility.

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

Consistent with other known members of the family of proteins, identified in column 5 of Table A, the NOVX polypeptides of the present invention show homology to, and contain domains that are characteristic of, other members of such protein families. Details of the sequence relatedness and domain analysis for each NOVX are presented in Example A.

The NOVX nucleic acids and polypeptides can also be used to screen for molecules, which inhibit or enhance NOVX activity or function. Specifically, the nucleic acids and polypeptides according to the invention may be used as targets for the identification of small molecules that modulate or inhibit diseases associated with the protein families listed in Table A.

The NOVX nucleic acids and polypeptides are also useful for detecting specific cell types. Details of the expression analysis for each NOVX are presented in Example C. Accordingly, the NOVX nucleic acids, polypeptides, antibodies and related compounds according to the invention will have diagnostic and therapeutic applications in the detection of a variety of diseases with differential expression in normal vs. diseased tissues, e.g. detection of a variety of cancers.

Additional utilities for NOVX nucleic acids and polypeptides according to the invention are disclosed herein.

NOVX Clones

NOVX nucleic acids and their encoded polypeptides are useful in a variety of applications and contexts. The various NOVX nucleic acids and polypeptides according to the invention are useful as novel members of the protein families according to the presence of domains and sequence relatedness to previously described proteins. Additionally, NOVX nucleic acids and polypeptides can also be used to identify proteins that are members of the family to which the NOVX polypeptides belong.

The NOVX genes and their corresponding encoded proteins are useful for preventing, treating or ameliorating medical conditions, e.g., by protein or gene therapy. Pathological conditions can be diagnosed by determining the amount of the new protein in a sample or by determining the presence of mutations in the new genes. Specific uses are described for each of the NOVX genes, based on the tissues in which they are most highly expressed. Uses include developing products for the diagnosis or treatment of a variety of diseases and disorders.

The NOVX nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed, as well as potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), and (v) a composition promoting tissue regeneration in vitro and in vivo (vi) a biological defense weapon.

In one specific embodiment, the invention includes an isolated polypeptide comprising an amino acid sequence selected from the group consisting of: (a) a mature form of the amino acid sequence selected from the group consisting of SEQ ID NO: 2n, wherein n is an integer between 1 and 124; (b) a variant of a mature form of the amino acid sequence selected from the group consisting of SEQ ID NO: 2n, wherein n is an integer between 1 and 124, wherein any amino acid in the mature form is changed to a different amino acid, provided that no more than 15% of the amino acid residues in the sequence of the mature form are so changed; (c) an amino acid sequence selected from the group consisting of SEQ ID NO: 2n, wherein n is an integer between 1 and 124; (d) a variant of the amino acid sequence selected from the group consisting of SEQ ID NO: 2n, wherein n is an integer between 1 and 124 wherein any amino acid specified in the chosen sequence is changed to a different amino acid, provided that no more than 15% of the amino acid residues in the sequence are so changed; and (e) a fragment of any of (a) through (d).

In another specific embodiment, the invention includes an isolated nucleic acid molecule comprising a nucleic acid sequence encoding a polypeptide comprising an amino acid sequence selected from the group consisting of: (a) a mature form of the amino acid sequence given SEQ ID NO: 2n, wherein n is an integer between 1 and 124; (b) a variant of a mature form of the amino acid sequence selected from the group consisting of SEQ ID NO: 2n, wherein n is an integer between 1 and 124 wherein any amino acid in the mature form of the chosen sequence is changed to a different amino acid, provided that no more than 15% of the amino acid residues in the sequence of the mature form are so changed; (c) the amino acid sequence selected from the group consisting of SEQ ID NO: 2n, wherein n is an integer between 1 and 124; (d) a variant of the amino acid sequence selected from the group consisting of SEQ ID NO: 2n, wherein n is an integer between 1 and 124, in which any amino acid specified in the chosen sequence is changed to a different amino acid, provided that no more than 15% of the amino acid residues in the sequence are so changed; (e) a nucleic acid fragment encoding at least a portion of a polypeptide comprising the amino acid sequence selected from the group consisting of SEQ ID NO: 2n, wherein n is an integer between 1 and 124 or any variant of said polypeptide wherein any amino acid of the chosen sequence is changed to a different amino acid, provided that no more than 10% of the amino acid residues in the sequence are so changed; and (f) the complement of any of said nucleic acid molecules.

In yet another specific embodiment, the invention includes an isolated nucleic acid molecule, wherein said nucleic acid molecule comprises a nucleotide sequence selected from the group consisting of: (a) the nucleotide sequence selected from the group consisting of SEQ ID NO: 2n−1, wherein n is an integer between 1 and 124; (b) a nucleotide sequence wherein one or more nucleotides in the nucleotide sequence selected from the group consisting of SEQ ID NO: 2n−1, wherein n is an integer between 1 and 124 is changed from that selected from the group consisting of the chosen sequence to a different nucleotide provided that no more than 15% of the nucleotides are so changed; (c) a nucleic acid fragment of the sequence selected from the group consisting of SEQ ID NO: 2n−1, wherein n is an integer between 1 and 124; and (d) a nucleic acid fragment wherein one or more nucleotides in the nucleotide sequence selected from the group consisting of SEQ ID NO: 2n−1, wherein n is an integer between 1 and 124 is changed from that selected from the group consisting of the chosen sequence to a different nucleotide provided that no more than 15% of the nucleotides are so changed.

NOVX Nucleic Acids and Polypeptides

One aspect of the invention pertains to isolated nucleic acid molecules that encode NOVX polypeptides or biologically active portions thereof. Also included in the invention are nucleic acid fragments sufficient for use as hybridization probes to identify NOVX-encoding nucleic acids (e.g., NOVX mRNAs) and fragments for use as PCR primers for the amplification and/or mutation of NOVX nucleic acid molecules. As used herein, the term “nucleic acid molecule” is intended to include DNA molecules (e.g., cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

A NOVX nucleic acid can encode a mature NOVX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product encoded by the corresponding gene. Alternatively, it may be defined as the polypeptide, precursor or proprotein encoded by an ORF described herein. The product “mature” form arises, by way of nonlimiting example, as a result of one or more naturally occurring processing steps that may take place within the cell (e.g., host cell) in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue 1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristylation or phosphorylation. In general, a mature polypeptide or protein may result from the operation of only one of these processes, or a combination of any of them.

The term “probe”, as utilized herein, refers to nucleic acid sequences of variable length, preferably between at least about 10 nucleotides (nt), about 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single-stranded or double-stranded and designed to have specificity in PCR, membrane-based hybridization technologies, or ELISA-like technologies.

The term “isolated” nucleic acid molecule, as used herein, is a nucleic acid that is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated NOVX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material, or culture medium, or of chemical precursors or other chemicals.

A nucleic acid molecule of the invention, e.g., a nucleic acid molecule having the nucleotide sequence of SEQ ID NO: 2n−1, wherein n is an integer between 1 and 124, or a complement of this nucleotide sequence, can be isolated using standard molecular biology techniques and the sequence information provided herein. Using all or a portion of the nucleic acid sequence of SEQ ID NO: 2n−1, wherein n is an integer between 1 and 124, as a hybridization probe, NOVX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template with appropriate oligonucleotide primers according to standard PCR amplification techniques. The nucleic acid so amplified can be cloned into an appropriate vector and characterized by DNA sequence analysis. Furthermore, oligonucleotides corresponding to NOVX nucleotide sequences can be prepared by standard synthetic techniques, e.g., using an automated DNA synthesizer.

As used herein, the term “oligonucleotide” refers to a series of linked nucleotide residues. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NO: 2n−1, wherein n is an integer between 1 and 124, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NO: 2n−1, wherein n is an integer between 1 and 124, or a portion of this nucleotide sequence (e.g., a fragment that can be used as a probe or primer or a fragment encoding a biologically-active portion of a NOVX polypeptide). A nucleic acid molecule that is complementary to the nucleotide sequence of SEQ ID NO: 2n−1, wherein n is an integer between 1 and 124, is one that is sufficiently complementary to the nucleotide sequence of SEQ ID NO: 2n−1, wherein n is an integer between 1 and 124, that it can hydrogen bond with few or no mismatches to the nucleotide sequence shown in SEQ ID NO: 2n−1, wherein n is an integer between 1 and 124, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermnediates.

A “fragment” provided herein is defined as a sequence of at least 6 (contiguous) nucleic acids or at least 4 (contiguous) amino acids, a length sufficient to allow for specific hybridization in the case of nucleic acids or for specific recognition of an epitope in the case of amino acids, and is at most some portion less than a full length sequence. Fragments may be derived from any contiguous portion of a nucleic acid or amino acid sequence of choice.

A full-length NOVX clone is identified as containing an ATG translation start codon and an in-frame stop codon. Any disclosed NOVX nucleotide sequence lacking an ATG start codon therefore encodes a truncated C-terminal fragment of the respective NOVX polypeptide, and requires that the corresponding full-length cDNA extend in the 5′ direction of the disclosed sequence. Any disclosed NOVX nucleotide sequence lacking an in-frame stop codon similarly encodes a truncated N-terminal fragment of the respective NOVX polypeptide, and requires that the corresponding full-length cDNA extend in the 3′ direction of the disclosed sequence.

A “derivative” is a nucleic acid sequence or amino acid sequence formed from the native compounds either directly, by modification or partial substitution. An “analog” is a nucleic acid sequence or amino acid sequence that has a structure similar to, but not identical to, the native compound, e.g. they differs from it in respect to certain components or side chains. Analogs may be synthetic or derived from a different evolutionary origin and may have a similar or opposite metabolic activity compared to wild type. A “homolog” is a nucleic acid sequence or amino acid sequence of a particular gene that is derived from different species.

Derivatives and analogs may be full length or other than full length. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the proteins under stringent, moderately stringent, or low stringent conditions. See e.g. Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences include those sequences coding for isoforms of NOVX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for a NOVX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human NOVX protein. Homologous nucleic acid sequences include those nucleic acid sequences that encode conservative amino acid substitutions (see below) in SEQ ID NO: 2n−1, wherein n is an integer between 1 and 124, as well as a polypeptide possessing NOVX biological activity. Various biological activities of the NOVX proteins are described below.

A NOVX polypeptide is encoded by the open reading frame (“ORF”) of a NOVX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bonafide cellular protein, a minimum size requirement is often set, e.g., a stretch of DNA that would encode a protein of 50 amino acids or more.

The nucleotide sequences determined from the cloning of the human NOVX genes allows for the generation of probes and primers designed for use in identifying and/or cloning NOVX homologues in other cell types, e.g. from other tissues, as well as NOVX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NO: 2n−1, wherein n is an integer between 1 and 124; or an anti-sense strand nucleotide sequence of SEQ ID NO: 2n−1, wherein n is an integer between 1 and 124; or of a naturally occurring mutant of SEQ ID NO: 2n−1, wherein n is an integer between 1 and 124.

Probes based on the human NOVX nucleotide sequences can be used to detect transcripts or genomic sequences encoding the same or homologous proteins. In various embodiments, the probe has a detectable label attached, e.g. the label can be a radioisotope, a fluorescent compound, an enzyme, or an enzyme co-factor. Such probes can be used as a part of a diagnostic test kit for identifying cells or tissues which mis-express a NOVX protein, such as by measuring a level of a NOVX-encoding nucleic acid in a sample of cells from a subject e.g., detecting NOVX mRNA levels or determining whether a genomic NOVX gene has been mutated or deleted.

“A polypeptide having a biologically-active portion of a NOVX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of NOVX” can be prepared by isolating a portion of SEQ ID NO: 2n−1, wherein n is an integer between 1 and 124, that encodes a polypeptide having a NOVX biological activity (the biological activities of the NOVX proteins are described below), expressing the encoded portion of NOVX protein (e.g., by recombinant expression in vitro) and assessing the activity of the encoded portion of NOVX.

NOVX Nucleic Acid and Polypeptide Variants

The invention further encompasses nucleic acid molecules that differ from the nucleotide sequences of SEQ ID NO: 2n−1, wherein n is an integer between 1 and 124, due to degeneracy of the genetic code and thus encode the same NOVX proteins as that encoded by the nucleotide sequences of SEQ ID NO: 2n−1, wherein n is an integer between 1 and 124. In another embodiment, an isolated nucleic acid molecule of the invention has a nucleotide sequence encoding a protein having an amino acid sequence of SEQ ID NO: 2n, wherein n is an integer between 1 and 124.

In addition to the human NOVX nucleotide sequences of SEQ ID NO: 2n−1, wherein n is an integer between 1 and 124, it will be appreciated by those skilled in the art that DNA sequence polymorphisms that lead to changes in the amino acid sequences of the NOVX polypeptides may exist within a population (e.g., the human population). Such genetic polymorphism in the NOVX genes may exist among individuals within a population due to natural allelic variation. As used herein, the terms “gene” and “recombinant gene” refer to nucleic acid molecules comprising an open reading frame (ORF) encoding a NOVX protein, preferably a vertebrate NOVX protein. Such natural allelic variations can typically result in 1-5% variance in the nucleotide sequence of the NOVX genes. Any and all such nucleotide variations and resulting amino acid polymorphisms in the NOVX polypeptides, which are the result of natural allelic variation and that do not alter the functional activity of the NOVX polypeptides, are intended to be within the scope of the invention.

Moreover, nucleic acid molecules encoding NOVX proteins from other species, and thus that have a nucleotide sequence that differs from a human SEQ ID NO: 2n−1, wherein n is an integer between 1 and 124, are intended to be within the scope of the invention. Nucleic acid molecules corresponding to natural allelic variants and homologues of the NOVX cDNAs of the invention can be isolated based on their homology to the human NOVX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NO: 2n−1, wherein n is an integer between 1 and 124. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least about 65% homologous to each other typically remain hybridized to each other.

Homologs (i.e., nucleic acids encoding NOVX proteins derived from species other than human) or other related sequences (e.g., paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to a sequence of SEQ ID NO: 2n−1, wherein n is an integer between 1 and 124, corresponds to a naturally-occurring nucleic acid molecule. As used herein, a “naturally-occuring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NO: 2n−1, wherein n is an integer between 1 and 124, or fragments, analogs or derivatives thereof, under conditions of moderate stringency is provided. A non-limiting example of moderate stringency hybridization conditions are hybridization in 6×SSC, 5×Reinhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, N.Y., and Krieger, 1990; GENE TRANSFER AND EXPRESSION, A LABORATORY MANUAL, Stockton Press, N.Y.

In a third embodiment, a nucleic acid that is hybridizable to the nucleic acid molecule comprising the nucleotide sequences of SEQ ID NO: 2n−1, wherein n is an integer between 1 and 124, or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g., as employed for cross-species hybridizations). See, e.g., Ausubel, et al. (eds.), 1993, CURRENT PROTOCOLS IN MOLECULAR BIOLOGY, John Wiley & Sons, N.Y., and Kriegler, 1990, GENE TRANSFER AND EEPRESSION, A LABORATORY MANUAL, Stockton Press, N.Y.; Shilo and Weinberg, 1981. Proc Natl Acad Sci USA 78: 6789-6792.

Conservative Mutations

In addition to naturally-occurring allelic variants of NOVX sequences that may exist in the population, the skilled artisan will further appreciate that changes can be introduced by mutation into the nucleotide sequences of SEQ ID NO: 2n−1, wherein n is an integer between 1 and 124, thereby leading to changes in the amino acid sequences of the encoded NOVX protein, without altering the functional ability of that NOVX protein. For example, nucleotide substitutions leading to amino acid substitutions at “non-essential” amino acid residues can be made in the sequence of SEQ ID NO: 2n, wherein n is an integer between 1 and 124. A “non-essential” amino acid residue is a residue that can be altered from the wild-type sequences of the NOVX proteins without altering their biological activity, whereas an “essential” amino acid residue is required for such biological activity. For example, amino acid residues that are conserved among the NOVX proteins of the invention are predicted to be particularly non-amenable to alteration. Amino acids for which conservative substitutions can be made are well-known within the art.

Another aspect of the invention pertains to nucleic acid molecules encoding NOVX proteins that contain changes in amino acid residues that are not essential for activity. Such NOVX proteins differ in amino acid sequence from SEQ ID NO: 2n−1, wherein n is an integer between 1 and 124, yet retain biological activity. In one embodiment, the isolated nucleic acid molecule comprises a nucleotide sequence encoding a protein, wherein the protein comprises an amino acid sequence at least about 40% homologous to the amino acid sequences of SEQ ID NO: 2n, wherein n is an integer between 1 and 124. Preferably, the protein encoded by the nucleic acid molecule is at least about 60% homologous to SEQ ID NO: 2n, wherein n is an integer between 1 and 124; more preferably at least about 70% homologous to SEQ ID NO: 2n, wherein n is an integer between 1 and 124; still more preferably at least about 80% homologous to SEQ ID NO: 2n, wherein n is an integer between 1 and 124; even more preferably at least about 90% homologous to SEQ ID NO: 2n, wherein n is an integer between 1 and 124; and most preferably at least about 95% homologous to SEQ ID NO: 2n, wherein n is an integer between 1 and 124.

An isolated nucleic acid molecule encoding a NOVX protein homologous to the protein of SEQ ID NO: 2n, wherein n is an integer between 1 and 124, can be created by introducing one or more nucleotide substitutions, additions or deletions into the nucleotide sequence of SEQ ID NO: 2n−1, wherein n is an integer between 1 and 124, such that one or more amino acid substitutions, additions or deletions are introduced into the encoded protein.

Mutations can be introduced any one of SEQ ID NO: 2n−1, wherein n is an integer between 1 and 124, by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g., lysine, arginine, histidine), acidic side chains (e.g., aspartic acid, glutamic acid), uncharged polar side chains (e.g., glycine, asparagine, glutamine, senne, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a predicted non-essential amino acid residue in the NOVX protein is replaced with another amino acid residue from the same side chain family. Alternatively, in another embodiment, mutations can be introduced randomly along all or part of a NOVX coding sequence, such as by saturation mutagenesis, and the resultant mutants can be screened for NOVX biological activity to identify mutants that retain activity. Following mutagenesis of a nucleic acid of SEQ ID NO: 2n−1, wherein n is an integer between 1 and 124, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, HFY, wherein the letters within each group represent the single letter amino acid code.

In one embodiment, a mutant NOVX protein can be assayed for (i) the ability to form protein:protein interactions with other NOVX proteins, other cell-surface proteins, or biologically-active portions thereof, (ii) complex formation between a mutant NOVX protein and a NOVX ligand; or (iii) the ability of a mutant NOVX protein to bind to an intracellular target protein or biologically-active portion thereof; (e.g. avidin proteins).

In yet another embodiment, a mutant NOVX protein can be assayed for the ability to regulate a specific biological function (e.g., regulation of insulin release).

Interfering RNA

In one aspect of the invention, NOVX gene expression can be attenuated by RNA interference. One approach well-known in the art is short interfering RNA (siRNA) mediated gene silencing where expression products of a NOVX gene are targeted by specific double stranded NOVX derived siRNA nucleotide sequences that are complementary to at least a 19-25 nt long segment of the NOVX gene transcript, including the 5′ untranslated (UT) region, the ORF, or the 3′ UT region. See, e.g., PCT applications WO00/44895, WO99/32619, WO01/75164, WO01/92513, WO 01/29058, WO01/89304, WO02/16620, and WO02/29858, each incorporated by reference herein in their entirety. Targeted genes can be a NOVX gene, or an upstream or downstream modulator of the NOVX gene. Nonlimiting examples of upstream or downstream modulators of a NOVX gene include, e.g., a transcription factor that binds the NOVX gene promoter, a kinase or phosphatase that interacts with a NOVX polypeptide, and polypeptides involved in a NOVX regulatory pathway.

According to the methods of the present invention, NOVX gene expression is silenced using short interfering RNA. A NOVX polynucleotide according to the invention includes a siRNA polynucleotide. Such a NOVX siRNA can be obtained using a NOVX polynucleotide sequence, for example, by processing the NOVX ribopolynucleotide sequence in a cell-free system, such as but not limited to a Drosophila extract, or by transcription of recombinant double stranded NOVX RNA or by chemical synthesis of nucleotide sequences homologous to a NOVX sequence. See, e.g., Tuschl, Zamore, Lehmann, Bartel and Sharp (1999), Genes & Dev. 13: 3191-3197, incorporated herein by reference in its entirety. When synthesized, a typical 0.2 micromolar-scale RNA synthesis provides about 1 milligram of siRNA, which is sufficient for 1000 transfection experiments using a 24-well tissue culture plate format.

The most efficient silencing is generally observed with siRNA duplexes composed of a 21-nt sense strand and a 21-nt antisense strand, paired in a manner to have a 2-nt 3′ overhang. The sequence of the 2-nt 3′ overhang makes an additional small contribution to the specificity of siRNA target recognition. The contribution to specificity is localized to the unpaired nucleotide adjacent to the first paired bases. In one embodiment, the nucleotides in the 3′ overhang are ribonucleotides. In an alternative embodiment, the nucleotides in the 3′ overhang are deoxyribonucleotides. Using 2′-deoxyribonucleotides in the 3′ overhangs is as efficient as using ribonucleotides, but deoxyribonucleotides are often cheaper to synthesize and are most likely more nuclease resistant.

A contemplated recombinant expression vector of the invention comprises a NOVX DNA molecule cloned into an expression vector comprising operatively-linked regulatory sequences flanking the NOVX sequence in a manner that allows for expression (by transcription of the DNA molecule) of both strands. An RNA molecule that is antisense to NOVX mRNA is transcribed by a first promoter (e.g., a promoter sequence 3′ of the cloned DNA) and an RNA molecule that is the sense strand for the NOVX mRNA is transcribed by a second promoter (e.g., a promoter sequence 5′ of the cloned DNA). The sense and antisense strands may hybridize in vivo to generate siRNA constructs for silencing of the NOVX gene. Alternatively, two constructs can be utilized to create the sense and anti-sense strands of a siRNA construct. Finally, cloned DNA can encode a construct having secondary structure, wherein a single transcript has both the sense and complementary antisense sequences from the target gene or genes. In an example of this embodiment, a hairpin RNAi product is homologous to all or a portion of the target gene. In another example, a hairpin RNAi product is a siRNA. The regulatory sequences flanking the NOVX sequence may be identical or may be different, such that their expression may be modulated independently, or in a temporal or spatial manner.

In a specific embodiment, siRNAs are transcribed intracellularly by cloning the NOVX gene templates into a vector containing, e.g., a RNA pol III transcription unit from the smaller nuclear RNA (snRNA) U6 or the human RNase P RNA H1. One example of a vector system is the GeneSuppressor™ RNA Interference kit (commercially available from lmgenex). The U6 and H1 promoters are members of the type III class of Pol III promoters. The +1 nucleotide of the U6-like promoters is always guanosine, whereas the +1 for H1 promoters is adenosine. The termination signal for these promoters is defined by five consecutive thyrmidines. The transcript is typically cleaved after the second uridine. Cleavage at this position generates a 3′ UU overhang in the expressed siRNA, which is similar to the 3′ overhangs of synthetic siRNAs. Any sequence less than 400 nucleotides in length can be transcribed by these promoter, therefore they are ideally suited for the expression of around 21-nucleotide siRNAs in, e.g., an approximately 50-nucleotide RNA stem-loop transcript.

A siRNA vector appears to have an advantage over synthetic siRNAs where long term knock-down of expression is desired. Cells transfected with a siRNA expression vector would experience steady, long-term mRNA inhibition. In contrast, cells transfected with exogenous synthetic siRNAs typically recover from mRNA suppression within seven days or ten rounds of cell division. The long-term gene silencing ability of siRNA expression vectors may provide for applications in gene therapy.

In general, siRNAs are chopped from longer dsRNA by an ATP-dependent ribonuclease called DICER. DICER is a member of the RNase III family of double-stranded RNA-specific endonucleases. The siRNAs assemble with cellular proteins into an endonuclease complex. In vitro studies in Drosophila suggest that the siRNAs/protein complex (siRNP) is then transferred to a second enzyme complex, called an RNA-induced silencing complex (RISC), which contains an endoribonuclease that is distinct from DICER. RISC uses the sequence encoded by the antisense siRNA strand to find and destroy mRNAs of complementary sequence. The siRNA thus acts as a guide, restricting the ribonuclease to cleave only mRNAs complementary to one of the two siRNA strands.

A NOVX mRNA region to be targeted by siRNA is generally selected from a desired NOVX sequence beginning 50 to 100 nt downstream of the start codon. Alternatively, 5′ or 3′ UTRs and regions nearby the start codon can be used but are generally avoided, as these may be richer in regulatory protein binding sites. UTR-binding proteins and/or translation initiation complexes may interfere with binding of the siRNP or RISC endonuclease complex. An initial BLAST homology search for the selected siRNA sequence is done against an available nucleotide sequence library to ensure that only one gene is targeted. Specificity of target recognition by siRNA duplexes indicate that a single point mutation located in the paired region of an siRNA duplex is sufficient to abolish target mRNA degradation. See, Elbashir et al. 2001 EMBO J. 20(23):6877-88. Hence, consideration should be taken to accommodate SNPs, polymorphisms, allelic variants or species-specific variations when targeting a desired gene.

In one embodiment, a complete NOVX siRNA experiment includes the proper negative control. A negative control siRNA generally has the same nucleotide composition as the NOVX siRNA but lack significant sequence homology to the genome. Typically, one would scramble, the nucleotide sequence of the NOVX siRNA and do a homology search to make sure it lacks homology to any other gene.

Two independent NOVX siRNA duplexes can be used to knock-down a target NOVX gene. This helps to control for specificity of the silencing effect. In addition, expression of two independent genes can be simultaneously knocked down by using equal concentrations of different NOVX siRNA duplexes, e.g., a NOVX siRNA and an siRNA for a regulator of a NOVX gene or polypeptide. Availability of siRNA-associating proteins is believed to be more limiting than target mRNA accessibility.

A targeted NOVX region is typically a sequence of two adenines (AA) and two thymidines (TT) divided by a spacer region of nineteen (N19) residues (e.g., AA(N19)TT). A desirable spacer region has a G/C-content of approximately 30% to 70%, and more preferably of about 50%. If the sequence AA(N19)TT is not present in the target sequence, an alternative target region would be AA(N21). The sequence of the NOVX sense siRNA corresponds to (N19)TT or N21, respectively. In the latter case, conversion of the 3′ end of the sense siRNA to TT can be performed if such a sequence does not naturally occur in the NOVX polynucleotide. The rationale for this sequence conversion is to generate a symmetric duplex with respect to the sequence composition of the sense and antisense 3′ overhangs. Symmetric 3′ overhangs may help to ensure that the siRNPs are formed with approximately equal ratios of sense and antisense target RNA-cleaving siRNPs. See, e.g., Elbashir, Lendeckel and Tuschl (2001). Genes & Dev. 15: 188-200, incorporated by reference herein in its entirely. The modification of the overhang of the sense sequence of the siRNA duplex is not expected to affect targeted mRNA recognition, as the antisense siRNA strand guides target recognition.

Alternatively, if the NOVX target mRNA does not contain a suitable AA(N21) sequence, one may search for the sequence NA(N21). Further, the sequence of the sense strand and antisense strand may still be synthesized as 5′ (N19)TT, as it is believed that the sequence of the 3′-most nucleotide of the antisense siRNA does not contribute to specificity. Unlike antisense or ribozyme technology, the secondary structure of the target mRNA does not appear to have a strong effect on silencing. See, Harborth, et al. (2001) J. Cell Science 114: 4557-4565, incorporated by reference in its entirety.

Transfection of NOVX siRNA duplexes can be achieved using standard nucleic acid transfection methods, for example, OLIGOFECTAMINE Reagent (commercially available from Invitrogen). An assay for NOVX gene silencing is generally performed approximately 2 days after transfection. No NOVX gene silencing has been observed in the absence of transfection reagent, allowing for a comparative analysis of the wild-type and silenced NOVX phenotypes. In a specific embodiment, for one well of a 24-well plate, approximately 0.84 μg of the siRNA duplex is generally sufficient. Cells are typically seeded the previous day, and are transfected at about 50% confluence. The choice of cell culture media and conditions are routine to those of skill in the art, and will vary with the choice of cell type. The efficiency of transfection may depend on the cell type, but also on the passage number and the confluency of the cells. The time and the manner of formation of siRNA-liposome complexes (e.g. inversion versus vortexing) are also critical. Low transfection efficiencies are the most frequent cause of unsuccessful NOVX silencing. The efficiency of transfection needs to be carefully examined for each new cell line to be used. Preferred cell are derived from a mammal, more preferably from a rodent such as a rat or mouse, and most preferably from a human. Where used for therapeutic treatment, the cells are preferentially autologous, although non-autologous cell sources are also contemplated as within the scope of the present invention.

For a control experiment, transfection of 0.84 μg single-stranded sense NOVX siRNA will have no effect on NOVX silencing, and 0.84 μg antisense siRNA has a weak silencing effect when compared to 0.84 μg of duplex siRNAs. Control experiments again allow for a comparative analysis of the wild-type and silenced NOVX phenotypes. To control for transfection efficiency, targeting of common proteins is typically performed, for example targeting of lamin A/C or transfection of a CMV-driven EGFP-expression plasmid (e.g. commercially available from Clontech). In the above example, a determination of the fraction of lamin A/C knockdown in cells is determined the next day by such techniques as immunofluorescence, Western blot, Northern blot or other similar assays for protein expression or gene expression. Lamin A/C monoclonal antibodies may be obtained from Santa Cruz Biotechnology.

Depending on the abundance and the half life (or turnover) of the targeted NOVX polynucleotide in a cell, a knock-down phenotype may become apparent after 1 to 3 days, or even later. In cases where no NOVX knock-down phenotype is observed, depletion of the NOVX polynucleotide may be observed by immunofluorescence or Western blotting. If the NOVX polynucleotide is still abundant after 3 days, cells need to be split and transferred to a fresh 24-well plate for re-transfection. If no knock-down of the targeted protein is observed, it may be desirable to analyze whether the target mRNA (NOVX or a NOVX upstream or downstream gene) was effectively destroyed by the transfected siRNA duplex. Two days after transfection, total RNA is prepared, reverse transcribed using a target-specific primer, and PCR-amplified with a primer pair covering at least one exon-exon junction in order to control for amplification of pre-mRNAs. RT/PCR of a non-targeted mRNA is also needed as control. Effective depletion of the mRNA yet undetectable reduction of target protein may indicate that a large reservoir of stable NOVX protein may exist in the cell. Multiple transfection in sufficiently long intervals may be necessary until the target protein is finally depleted to a point where a phenotype may become apparent. If multiple transfection steps are required, cells are split 2 to 3 days after transfection. The cells may be transfected immediately after splitting.

An inventive therapeutic method of the invention contemplates administering a NOVX siRNA construct as therapy to compensate for increased or aberrant NOVX expression or activity. The NOVX ribopolynucleotide is obtained and processed into siRNA fragments, or a NOVX siRNA is synthesized, as described above. The NOVX siRNA is administered to cells or tissues using known nucleic acid transfection techniques, as described above. A NOVX siRNA specific for a NOVX gene will decrease or knockdown NOVX transcription products, which will lead to reduced NOVX polypeptide production, resulting in reduced NOVX polypeptide activity in the cells or tissues.

The present invention also encompasses a method of treating a disease or condition associated with the presence of a NOVX protein in an individual comprising administering to the individual an RNAi construct that targets the MRNA of the protein (the mRNA that encodes the protein) for degradation. A specific RNAi construct includes a siRNA or a double stranded gene transcript that is processed into siRNAs. Upon treatment, the target protein is not produced or is not produced to the extent it would be in the absence of the treatment.

Where the NOVX gene function is not correlated with a known phenotype, a control sample of cells or tissues from healthy individuals provides a reference standard for determining NOVX expression levels. Expression levels are detected using the assays described, e.g., RT-PCR, Northern blotting, Western blotting, ELISA, and the like. A subject sample of cells or tissues is taken from a mammal, preferably a human subject, suffering from a disease state. The NOVX ribopolynucleotide is used to produce siRNA constructs, that are specific for the NOVX gene product. These cells or tissues are treated by administering NOVX siRNA's to the cells or tissues by methods described for the transfection of nucleic acids into a cell or tissue, and a change in NOVX polypeptide or polynuclectide expression is observed in the subject sample relative to the control sample, using the assays described. This NOVX gene knockdown approach provides a rapid method for determination of a NOVX minus (NOVX⁻) phenotype in the treated subject sample. The NOVX⁻phenotype observed in the treated subject sample thus serves as a marker for monitoring the course of a disease state during treatment.

In specific embodiments, a NOVX siRNA is used in therapy. Methods for the generation and use of a NOVX siRNA are known to those skilled in the art. Example techniques are provided below.

Production of RNAs

Sense RNA (ssRNA) and antisense RNA (asRNA) of NOVX are produced using known methods such as transcription in RNA expression vectors. In the initial experiments, the sense and antisense RNA are about 500 bases in length each. The produced ssRNA and asRNA (0.5 μM) in 10 mM Tris-HCl (pH 7.5) with 20 mM NaCl were heated to 95° C. for 1 min then cooled and annealed at room temperature for 12 to 16 h. The RNAs are precipitated and resuspended in lysis buffer (below). To monitor annealing, RNAs are electrophoresed in a 2% agarose gel in TBE buffer and stained with ethidium bromide. See, e.g., Sambrook et al., Molecular Cloning. Cold Spring Harbor Laboratory Press, Plainview, N.Y. (1989).

Lysate Preparation

Untreated rabbit reticulocyte lysate (Ambion) are assembled according to the manufacturer's directions. dsRNA is incubated in the lysate at 30° C. for 10 min prior to the addition of mRNAs. Then NOVX mRNAs are added and the incubation continued for an additional 60 min. The molar ratio of double stranded RNA and mRNA is about 200:1. The NOVX mRNA is radiolabeled (using known techniques) and its stability is monitored by gel electrophoresis.

In a parallel experiment made with the same conditions, the double stranded RNA is internally radiolabeled with a ³²P-ATP. Reactions are stopped by the addition of 2×proteinase K buffer and deproteinized as described previously (Tuschl et al., Genes Dev., 13:3191-3197 (1999)). Products are analyzed by electrophoresis in 15% or 18% polyacrylamide sequencing gels using appropriate RNA standards. By monitoring the gels for radioactivity, the natural production of 10 to 25 nt RNAs from the double stranded RNA can be determined.

The band of double stranded RNA, about 21-23 bps, is eluded. The efficacy of these 21-23 mers for suppressing NOVX transcription is assayed in vitro using the same rabbit reticulocyte assay described above using 50 nanomolar of double stranded 21-23 mer for each assay. The sequence of these 21-23 mers is then determined using standard nucleic acid sequencing techniques.

RNA Preparation

21 nt RNAs, based on the sequence determined above, are chemically synthesized using Expedite RNA phosphoramidites and thymidine phosphoramidite (Proligo, Germany). Synthetic oligonucleotides are deprotected and gel-purified (Elbashir, Lendeckel, & Tuschl, Genes & Dev. 15, 188-200 (2001)), followed by Sep-Pak C18 cartridge (Waters, Milford, Mass., USA) purification (Tuschl, et al., Biochemistry, 32:11658-11668 (1993)).

These RNAs (20 μM) single strands are incubated in annealing buffer (100 mM potassium acetate, 30 mM HEPES-KOH at pH 7.4, 2 mM magnesium acetate) for 1 min at 90° C. followed by 1 h at 37°C.

Cell Culture

A cell culture known in the art to regularly express NOVX is propagated using standard conditions. 24 hours before transfection, at approx. 80% confluency, the cells are trypsinized and diluted 1:5 with fresh medium without antibiotics (1-3×105 cells/ml) and transferred to 24-well plates (500 ml/well). Transfection is performed using a commercially available lipofection kit and NOVX expression is monitored using standard techniques with positive and negative control. A positive control is cells that naturally express NOVX while a negative control is cells that do not express NOVX. Base-paired 21 and 22 nt siRNAs with overhanging 3′ ends mediate efficient sequence-specific mRNA degradation in lysates and in cell culture. Different concentrations of siRNAs are used. An efficient concentration for suppression in vitro in mammalian culture is between 25 nM to 100 nM final concentration. This indicates that siRNAs are effective at concentrations that are several orders of magnitude below the concentrations applied in conventional antisense or ribozyme gene targeting experiments.

The above method provides a way both for the deduction of NOVX siRNA sequence and the use of such siRNA for in vitro suppression. In vivo suppression may be performed using the same siRNA using well known in vivo transfection or gene therapy transfection techniques.

Antisense Nucleic Acids

Another aspect of the invention pertains to isolated antisense nucleic acid molecules that are hybridizable to or complementary to the nucleic acid molecule comprising the nucleotide sequence of SEQ ID NO: 2n−1, wherein n is an integer between 1 and 124, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire NOVX coding strand, or to only a portion thereof. Nucleic acid molecules encoding fragments, homologs, derivatives and analogs of a NOVX protein of SEQ ID NO: 2n, wherein n is an integer between 1 and 124, or antisense nucleic acids complementary to a NOVX nucleic acid sequence of SEQ ID NO: 2n−1, wherein n is an integer between 1 and 124, are additionally provided.

In one embodiment, an antisense nucleic acid molecule is antisense to a “coding region” of the coding strand of a nucleotide sequence encoding a NOVX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the NOVX protein. The term “noncoding region” refers to 5′ and 3′ sequences which flank the coding region that are not translated into amino acids (i.e., also referred to as 5′ and 3′ untranslated regions).

Given the coding strand sequences encoding the NOVX protein disclosed herein, antisense nucleic acids of the invention can be designed according to the rules of Watson and Crick or Hoogsteen base pairing. The antisense nucleic acid molecule can be complementary to the entire coding region of NOVX mRNA, but more preferably is an oligonucleotide that is antisense to only a portion of the coding or noncoding region of NOVX mRNA. For example, the antisense oligonucleotide can be complementary to the region surrounding the translation start site of NOVX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-carboxymethylaminomethyl-2-thiouridine, 5-(carboxyhydroxylmethyl) uracil, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 5-methoxyuracil, 3-methylcytosine, 5-methylcytosine, N6-adenine, 7-methylguanine, 5-methylaminomethyluracil, 5-methoxyaminomethyl-2-thiouracil, 2-thiouracil, 4-thiouracil, beta-D-mannosylqueosine, 5′-methoxycarboxymethyluracil, 2-methylthio-N6-isopentenyladenine, uracil-5-oxyacetic acid (v), wybutoxosine, pseudouracil, queosine, 2-thiocytosine, 5-methyl-2-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genomic DNA encoding a NOVX protein to thereby inhibit expression of the protein (e.g., by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e.g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an (x-anomeric nucleic acid molecule. An α-anomeric nucleic acid molecule forms specific double-stranded hybrids with complementary RNA in which, contrary to the usual 0-units, the strands run parallel to each other. See, e.g., Gaultier, et al., 1987. Nucl. Acids Res. 15: 6625-6641. The antisense nucleic acid molecule can also comprise a 2′-o-methylribonucleotide (See, e.g., Inoue, et al. 1987. Nucl. Acids Res. 15: 6131-6148) or a chimeric RNA-DNA analogue (See, e.g., Inoue, et al., 1987. FEBS Lett. 215: 327-330.

Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g., hammerhead ribozymes as described in Haselhoff and Gerlach 1988. Nature 334: 585-591) can be used to catalytically cleave NOVX mRNA transcripts to thereby inhibit translation of NOVX mRNA. A ribozyme having specificity for a NOVX-encoding nucleic acid can be designed based upon the nucleotide sequence of a NOVX cDNA disclosed herein (i.e., SEQ ID NO: 2n−1, wherein n is an integer between 1 and 124). For example, a derivative of a Tetrahymena L-19 IVS RNA can be constructed in which the nucleotide sequence of the active site is complementary to the nucleotide sequence to be cleaved in a NOVX-encoding mRNA. See, e.g., U.S. Pat. No. 4,987,071 to Cech, et al. and U.S. Pat. No. 5,116,742 to Cech, et al. NOVX mRNA can also be used to select a catalytic RNA having a specific ribonuclease activity from a pool of RNA molecules. See, e.g., Bartel et al., (1993) Science 261:1411-1418.

Alternatively, NOVX gene expression can be inhibited by targeting nucleotide sequences complementary to the regulatory region of the NOVX nucleic acid (e.g., the NOVX promoter and/or enhancers) to form triple helical structures that prevent transcription of the NOVX gene in target cells. See, e.g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the NOVX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleotide bases are retained. The neutral backbone of PNAs has been shown to allow for specific hybridization to DNA and RNA under conditions of low ionic strength. The synthesis of PNA oligomer can be performned using standard solid phase peptide synthesis protocols as described in Hyrup, et al., 1996. supra; Perry-O'Keefe, et al., 1996. Proc. Natl. Acad. Sci. USA 93: 14670-14675.

PNAs of NOVX can be used in therapeutic and diagnostic applications. For example, PNAs can be used as antisense or antigene agents for sequence-specific modulation of gene expression by, e.g., inducing transcription or translation arrest or inhibiting replication. PNAs of NOVX can also be used, for example, in the analysis of single base pair mutations in a gene (e.g., PNA directed PCR clamping; as artificial restriction enzymes when used in combination with other enzymes, e.g., S₁ nucleases (See, Hyrup, et al., 1996.supra); or as probes or primers for DNA sequence and hybridization (See, Hyrup, et al., 1996, supra; Perry-O'Keefe, et al., 1996. supra).

In another embodiment, PNAs of NOVX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of NOVX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleotide bases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. See, e.g., Finn, et al., 1996. supra. Alternatively, chimeric molecules can be synthesized with a 5′ DNA segment and a 3′ PNA segment. See, e.g., Petersen, et al., 1975. Bioorg. Med. Chem. Lett. 5: 1119-11124.

In other embodiments, the oligonucleotide may include other appended groups such as peptides (e.g., for targeting host cell receptors in vivo), or agents facilitating transport across the cell membrane (see, e.g., Letsinger, et al., 1989. Proc. Nati. Acad. Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharnn. Res. 5: 539-549). To this end, the oligonucleotide may be conjugated to another molecule, e.g., a peptide, a hybridization triggered cross-linking agent, a transport agent, a hybridization-triggered cleavage agent, and the like.

NOVX Polypeptides

A polypeptide according to the invention includes a polypeptide including the amino acid sequence of NOVX polypeptides whose sequences are provided in any one of SEQ ID NO: 2n, wherein n is an integer between 1 and 124. The invention also includes a mutant or variant protein any of whose residues may be changed from the corresponding residues shown in any one of SEQ ID NO: 2n, wherein n is an integer between 1 and 124, while still encoding a protein that maintains its NOVX activities and physiological functions, or a functional fragment thereof.

In general, a NOVX variant that preserves NOVX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated NOVX proteins, and biologically-active portions thereof, or derivatives, fragments, analogs or homologs thereof. Also provided are polypeptide fragments suitable for use as immunogens to raise anti-NOVX antibodies. In one embodiment, native NOVX proteins can be isolated from cells or tissue sources by an appropriate purification scheme using standard protein purification techniques. In another embodiment, NOVX proteins are produced by recombinant DNA techniques. Alternative to recombinant expression, a NOVX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the NOVX protein is derived, or substantially free from chemical precursors or other chemicals when chemically synthesized. The language “substantially free of cellular material” includes preparations of NOVX proteins in which the protein is separated from cellular components of the cells from which it is isolated or recombinantly-produced. In one embodiment, the language “substantially free of cellular material” includes preparations of NOVX proteins having less than about 30% (by dry weight) of non-NOVX proteins (also referred to herein as a “contaminating protein”), more preferably less than about 20% of non-NOVX proteins, still more preferably less than about 10% of non-NOVX proteins, and most preferably less than about 5% of non-NOVX proteins. When the NOVX protein or biologically-active portion thereof is recombinantly-produced, it is also preferably substantially free of culture medium, i.e., culture medium represents less than about 20%, more preferably less than about 10%, and most preferably less than about 5% of the volume of the NOVX protein preparation.

The language “substantially free of chemical precursors or other chemicals” includes preparations of NOVX proteins in which the protein is separated from chemical precursors or other chemicals that are involved in the synthesis of the protein. In one embodiment, the language “substantially free of chemical precursors or other chemicals” includes preparations of NOVX proteins having less than about 30% (by dry weight) of chemical precursors or non-NOVX chemicals, more preferably less than about 20% chemical precursors or non-NOVX chemicals, still more preferably less than about 10% chemical precursors or non-NOVX chemicals, and most preferably less than about 5% chemical precursors or non-NOVX chemicals.

Biologically-active portions of NOVX proteins include peptides comprising amino acid sequences sufficiently homologous to or derived from the amino acid sequences of the NOVX proteins (e.g., the amino acid sequence of SEQ ID NO: 2n, wherein n is an integer between 1 and 124) that include fewer amino acids than the full-length NOVX proteins, and exhibit at least one activity of a NOVX protein. Typically, biologically-active portions comprise a domain or motif with at least one activity of the NOVX protein. A biologically-active portion of a NOVX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native NOVX protein.

In an embodiment, the NOVX protein has an amino acid sequence of SEQ ID NO: 2n, wherein n is an integer between 1 and 124. In other embodiments, the NOVX protein is substantially homologous to SEQ ID NO: 2n, wherein n is an integer between 1 and 124, and retains the functional activity of the protein of SEQ ID NO: 2n, wherein n is an integer between 1 and 124, yet differs in amino acid sequence due to natural allelic variation or mutagenesis, as described in detail, below. Accordingly, in another embodiment, the NOVX protein is a protein that comprises an amino acid sequence at least about 45% homologous to the amino acid sequence of SEQ ID NO: 2n, wherein n is an integer between 1 and 124, and retains the functional activity of the NOVX proteins of SEQ ID NO: 2n, wherein n is an integer between 1 and 124.

Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence of SEQ ID NO: 2n−1, wherein n is an integer between 1 and 124.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e.g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

Chimeric and Fusion Proteins

The invention also provides NOVX chimeric or fusion proteins. As used herein, a NOVX “chimeric protein” or “fusion protein” comprises a NOVX polypeptide operatively-linked to a non-NOVX polypeptide. An “NOVX polypeptide” refers to a polypeptide having an amino acid sequence corresponding to a NOVX protein of SEQ ID NO: 2n, wherein n is an integer between 1 and 124, whereas a “non-NOVX polypeptide” refers to a polypeptide having an amino acid sequence corresponding to a protein that is not substantially homologous to the NOVX protein, e.g., a protein that is different from the NOVX protein and that is derived from the same or a different organism. Within a NOVX fusion protein the NOVX polypeptide can correspond to all or a portion of a NOVX protein. In one embodiment, a NOVX fusion protein comprises at least one biologically-active portion of a NOVX protein. In another embodiment, a NOVX fusion protein comprises at least two biologically-active portions of a NOVX protein. In yet another embodiment, a NOVX fusion protein comprises at least three biologically-active portions of a NOVX protein. Within the fusion protein, the term “operatively-linked” is intended to indicate that the NOVX polypeptide and the non-NOVX polypeptide are fused in-frame with one another. The non-NOVX polypeptide can be fused to the N-terminus or C-terminus of the NOVX polypeptide.

In one embodiment, the fusion protein is a GST-NOVX fusion protein in which the NOVX sequences are fused to the C-terminus of the GST (glutathione S-transferase) sequences. Such fusion proteins can facilitate the purification of recombinant NOVX polypeptides.

In another embodiment, the fusion protein is a NOVX protein containing a heterologous signal sequence at its N-terminus. In certain host cells (e.g., mammalian host cells), expression and/or secretion of NOVX can be increased through use of a heterologous signal sequence.

In yet another embodiment, the fusion protein is a NOVX-immunoglobulin fusion protein in which the NOVX sequences are fused to sequences derived from a member of the immunoglobulin protein family. The NOVX-immunoglobulin fusion proteins of the invention can be incorporated into pharmaceutical compositions and administered to a subject to inhibit an interaction between a NOVX ligand and a NOVX protein on the surface of a cell, to thereby suppress NOVX-mediated signal transduction in vivo. The NOVX-immunoglobulin fusion proteins can be used to affect the bioavailability of a NOVX cognate ligand. Inhibition of the NOVX ligand&NOVX interaction may be useful therapeutically for both the treatment of proliferative and differentiative disorders, as well as modulating (e.g. promoting or inhibiting) cell survival. Moreover, the NOVX-immunoglobulin fusion proteins of the invention can be used as immunogens to produce anti-NOVX antibodies in a subject, to purify NOVX ligands, and in screening assays to identify molecules that inhibit the interaction of NOVX with a NOVX ligand.

A NOVX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g., a GST polypeptide). A NOVX-encoding nucleic acid can be cloned into such an expression vector such that the fusion moiety is linked in-frame to the NOVX protein.

NOVX Agonists and Antagonists

The invention also pertains to variants of the NOVX proteins that function as either NOVX agonists (i.e., mimetics) or as NOVX antagonists. Variants of the NOVX protein can be generated by mutagenesis (e.g., discrete point mutation or truncation of the NOVX protein). An agonist of the NOVX protein can retain substantially the same, or a subset of, the biological activities of the naturally occurring form of the NOVX protein. An antagonist of the NOVX protein can inhibit one or more of the activities of the naturally occurring form of the NOVX protein by, for example, competitively binding to a downstream or upstream member of a cellular signaling cascade which includes the NOVX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the NOVX proteins.

Variants of the NOVX proteins that function as either NOVX agonists (i.e., mimetics) or as NOVX antagonists can be identified by screening combinatorial libraries of mutants (e.g., truncation mutants) of the NOVX proteins for NOVX protein agonist or antagonist activity. In one embodiment, a variegated library of NOVX variants is generated by combinatorial mutagenesis at the nucleic acid level and is encoded by a variegated gene library. A variegated library of NOVX variants can be produced by, for example, enzymatically ligating a mixture of synthetic oligonucleotides into gene sequences such that a degenerate set of potential NOVX sequences is expressible as individual polypeptides, or alternatively, as a set of larger fusion proteins (e.g., for phage display) containing the set of NOVX sequences therein. There are a variety of methods which can be used to produce libraries of potential NOVX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential NOVX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

Polypeptide Libraries

In addition, libraries of fragments of the NOVX protein coding sequences can be used to generate a variegated population of NOVX fragments for screening and subsequent selection of variants of a NOVX protein. In one embodiment, a library of coding sequence fragments can be generated by treating a double stranded PCR fragment of a NOVX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the NOVX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of NOVX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify NOVX variants. See, e.g., Arkin and Yourvan, 1992. Proc. Natl. Acad. Sci. USA 89: 7811-7815; Delgrave, et al., 1993. Protein Engineering 6:327-331.

Anti-NOVX Antibodies

Included in the invention are antibodies to NOVX proteins, or fragments of NOVX proteins. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein, such as an amino acid sequence of SEQ ID NO: 2n, wherein n is an integer between 1 and 124, and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of NOVX that is located on the surface of the protein, e.g., a hydrophilic region. A hydrophobicity analysis of the human NOVX protein sequence will indicate which regions of a NOVX polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. A NOVX polypeptide or a fragment thereof comprises at least one antigenic epitope. An anti-NOVX antibody of the present invention is said to specifically bind to antigen NOVX when the equilibrium binding constant (KD) is ≦1 μM, preferably ≦100 nM, more preferably ≦10 nM, and most preferably ≦100 pM to about 1 pM, as measured by assays such as radioligand binding assays or similar assays known to those skilled in the art.

A protein of the invention, or a derivative, fragment, analog, homolog or ortholog thereof, may be utilized as an immunogen in the generation of antibodies that immunospecifically bind these protein components.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, NY, incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, California and the American Type Culture Collection, Manassas, Virginia. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding,1986). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al,(Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof: Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) F, fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994).

Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcyRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA, or TETA. Another bispecific antibody of interest binds the protein antigen described herein and further binds tissue factor (TF).

Heteroconjugate Antibodies

Heteroconjugate antibodies are also within the scope of the present invention. Heteroconjugate antibodies are composed of two covalently joined antibodies. Such antibodies have, for example, been proposed to target immune system cells to unwanted cells (U.S. Pat. No. 4,676,980), and for treatment of HIV infection (WO 91/00360; WO 92/200373; EP 03089). It is contemplated that the antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

Effector Function Engineering

It can be desirable to modify the antibody of the invention with respect to effector function, so as to enhance, e.g., the effectiveness of the antibody in treating cancer. For example, cysteine residue(s) can be introduced into the Fc region, thereby allowing interchain disulfide bond formation in this region. The homodimeric antibody thus generated can have improved internalization capability and/or increased complement-mediated cell killing and antibody-dependent cellular cytotoxicity (ADCC). See Caron et al., J. Exp Med., 176: 1191-1195 (1992) and Shopes, J. Immunol., 148: 2918-2922 (1992). Homodimeric antibodies with enhanced anti-tumor activity can also be prepared using heterobifunctional cross-linkers as described in Wolff et al. Cancer Research, 53: 2560-2565 (1993). Alternatively, an antibody can be engineered that has dual Fc regions and can thereby have enhanced complement lysis and ADCC capabilities. See Stevenson et al., Anti-Cancer Drug Design, 3: 219-230 (1989).

Immunoconjugates

The invention also pertains to immunoconjugates comprising an antibody conjugated to a cytotoxic agent such as a chemotherapeutic agent, toxin (e.g., an enzymatically active toxin of bacterial, fungal, plant, or animal origin, or fragments thereof), or a radioactive isotope (i.e., a radioconjugate).

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and ¹⁸⁶Re.

Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

In another embodiment, the antibody can be conjugated to a “receptor” (such streptavidin) for utilization in tumor pretargeting wherein the antibody-receptor conjugate is administered to the patient, followed by removal of unbound conjugate from the circulation using a clearing agent and then administration of a “ligand” (e.g., avidin) that is in turn conjugated to a cytotoxic agent.

Immunoliposomes

The antibodies disclosed herein can also be formnulated as immunoliposomes. Liposomes containing the antibody are prepared by methods known in the art, such as described in Epstein et al., Proc. Natl. Acad. Sci. USA, 82: 3688 (1985); Hwang et al., Proc. Natl Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al.,J. Biol. Chem., 257: 286-288 (1982) via a disulfide-interchange reaction. A chemotherapeutic agent (such as Doxorubicin) is optionally contained within the liposome. See Gabizon et al., J. National Cancer Inst., 81(19): 1484 (1989).

Diagnostic Applications of Antibodies Directed Against the Proteins of the Invention

In one embodiment, methods for the screening of antibodies that possess the desired specificity include, but are not limited to, enzyme linked immunosorbent assay (ELISA) and other immunologically mediated techniques known within the art. In a specific embodiment, selection of antibodies that are specific to a particular domain of an NOVX protein is facilitated by generation of hybridomas that bind to the fragment of an NOVX protein possessing such a domain. Thus, antibodies that are specific for a desired domain within an NOVX protein, or derivatives, fragments, analogs or homologs thereof, are also provided herein.

Antibodies directed against a NOVX protein of the invention may be used in methods known within the art relating to the localization and/or quantitation of a NOVX protein (e.g., for use in measuring levels of the NOVX protein within appropriate physiological samples, for use in diagnostic methods, for use in imaging the protein, and the like). In a given embodiment, antibodies specific to a NOVX protein, or derivative, fragment, analog or homolog thereof, that contain the antibody derived antigen binding domain, are utilized as pharmacologically active compounds (referred to hereinafter as “Therapeutics”).

An antibody specific for a NOVX protein of the invention (e.g., a monoclonal antibody or a polyclonal antibody) can be used to isolate a NOVX polypeptide by standard techniques, such as immunoaffinity, chromatography or immunoprecipitation. An antibody to a NOVX polypeptide can facilitate the purification of a natural NOVX antigen from cells, or of a recombinantly produced NOVX antigen expressed in host cells. Moreover, such an anti-NOVX antibody can be used to detect the antigenic NOVX protein (e.g., in a cellular lysate or cell supernatant) in order to evaluate the abundance and pattern of expression of the antigenic NOVX protein. Antibodies directed against a NOVX protein can be used diagnostically to monitor protein levels in tissue as part of a clinical testing procedure, e.g., to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidintbiotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or³H.

Antibody Therapeutics

Antibodies of the invention, including polyclonal, monoclonal, humanized and fully human antibodies, may used as therapeutic agents. Such agents will generally be employed to treat or prevent a disease or pathology in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous ligand to which it naturally binds. In this case, the antibody binds to the target and masks a binding site of the naturally occurring ligand, wherein the ligand serves as an effector molecule. Thus the receptor mediates a signal transduction pathway for which ligand is responsible.

Alternatively, the effect may be one in which the antibody elicits a physiological result by virtue of binding to an effector binding site on the target molecule. In this case the target, a receptor having an endogenous ligand which may be absent or defective in the disease or pathology, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen, and will also depend on the rate at which an administered antibody is depleted from the free volume other subject to which it is administered. Common ranges for therapeutically effective dosing of an antibody or antibody fragment of the invention may be, by way of nonlimiting example, from about 0.1 mg/kg body weight to about 50 mg/kg body weight. Common dosing frequencies may range, for example, from twice daily to once a week.

Pharmaceutical Compositions of Antibodies

Antibodies specifically binding a protein of the invention, as well as other molecules identified by the screening assays disclosed herein, can be administered for the treatment of various disorders in the form of pharmaceutical compositions. Principles and considerations involved in preparing such compositions, as well as guidance in the choice of components are provided, for example, in Remington: The Science And Practice Of Pharmacy 19th ed. (Alfonso R. Gennaro, et al., editors) Mack Pub. Co., Easton, Pa.: 1995; Drug Absorption Enhancement: Concepts, Possibilities, Limitations, And Trends, Harwood Academic Publishers, Langhorne, Pa., 1994; and Peptide And Protein Drug Delivery (Advances In Parenteral Sciences, Vol. 4), 1991, M. Dekker, New York.

If the antigenic protein is intracellular and whole antibodies are used as inhibitors, internalizing antibodies are preferred. However, liposomes can also be used to deliver the antibody, or an antibody fragment, into cells. Where antibody fragments are used, the smallest inhibitory fragment that specifically binds to the binding domain of the target protein is preferred. For example, based upon the variable-region sequences of an antibody, peptide molecules can be designed that retain the ability to bind the target protein sequence. Such peptides can be synthesized chemically and/or produced by recombinant DNA technology. See, e.g., Marasco et al., Proc. Natl. Acad. Sci. USA, 90: 7889-7893 (1993). The formulation herein can also contain more than one active compound as necessary for the particular indication being treated, preferably those with complementary activities that do not adversely affect each other. Alternatively, or in addition, the composition can comprise an agent that enhances its function, such as, for example, a cytotoxic agent, cytokine, chemotherapeutic agent, or growth-inhibitory agent. Such molecules are suitably present in combination in amounts that are effective for the purpose intended.

The active ingredients can also be entrapped in microcapsules prepared, for example, by coacervation techniques or by interfacial polymerization, for example, hydroxymethylcellulose or gelatin-microcapsules and poly-(methylmethacrylate) microcapsules, respectively, in colloidal drug delivery systems (for example, liposomes, albumin microspheres, microemulsions, nano-particles, and nanocapsules) or in macroemulsions.

The formulations to be used for in vivo administration must be sterile. This is readily accomplished by filtration through sterile filtration membranes.

Sustained-release preparations can be prepared. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods.

ELISA Assay

An agent for detecting an analyte protein is an antibody capable of binding to an analyte protein, preferably an antibody with a detectable label. Antibodies can be polyclonal, or more preferably, monoclonal. An intact antibody, or a fragment thereof (e.g., F_{ab} or F_{(ab)2}) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. Included within the usage of the term “biological sample”, therefore, is blood and a fraction or component of blood including blood serum, blood plasma, or lymph. That is, the detection method of the invention can be used to detect an analyte mRNA, protein, or genomic DNA in a biological sample in vitro as well as in vivo. For example, in vitro techniques for detection of an analyte mRNA include Northern hybridizations and in situ hybridizations. In vitro techniques for detection of an analyte protein include enzyme linked immunosorbent assays (ELISAs), Western blots, immunoprecipitations, and immunofluorescence. In vitro techniques for detection of an analyte genomic DNA include Southern hybridizations. Procedures for conducting immunoassays are described, for example in “ELISA: Theory and Practice: Methods in Molecular Biology”, Vol. 42, J. R. Crowther (Ed.) Human Press, Totowa, N.J., 1995; “Immunoassay”, E. Diamandis and T. Christopoulus, Academic Press, Inc., San Diego, Calif., 1996; and “Practice and Thory of Enzyme Immunoassays”, P. Tijssen, Elsevier Science Publishers, Amsterdam, 1985. Furthermore, in vivo techniques for detection of an analyte protein include introducing into a subject a labeled anti-an analyte protein antibody. For example, the antibody can be labeled with a radioactive marker whose presence and location in a subject can be detected by standard imaging techniques.

NOVX Recombinant Expression Vectors and Host Cells

Another aspect of the invention pertains to vectors, preferably expression vectors, containing a nucleic acid encoding a NOVX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmid” and “vector” can be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g., in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g., NOVX proteins, mutant forms of NOVX proteins, fusion proteins, etc.).

The recombinant expression vectors of the invention can be designed for expression of NOVX proteins in prokaryotic or eukaryotic cells. For example, NOVX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS mN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E. coli (see, e.g., Wada, et al., 1992. Nucl. Acids Res. 20: 2111-2118). Such alteration of nucleic acid sequences of the invention can be carried out by standard DNA synthesis techniques.

In another embodiment, the NOVX expression vector is a yeast expression vector. Examples of vectors for expression in yeast Saecharomyces cerivisae include pYepSec1 (Baldari, et al., 1987. EMBO J. 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, NOVX can be expressed in insect cells using baculovirus expression vectors. Baculovirus vectors available for expression of proteins in cultured insect cells (e.g., SF9 cells) include the pAc series (Smith, et al., 1983. Mol. Cell. Biol. 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e.g., the neurofilament promoter; Byrne and Ruddle, 1989. Proc. Natl. Acad. Sci. USA 86: 5473-5477), pancreas-specific promoters (Edlund, et al., 1985. Science 230: 912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the (x-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to NOVX mRNA. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, NOVX protein can be expressed in bacterial cells such as E. coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding NOVX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e., express) NOVX protein. Accordingly, the invention further provides methods for producing NOVX protein using the host cells of the invention. In one embodiment, the method comprises culturing the host cell of invention (into which a recombinant expression vector encoding NOVX protein has been introduced) in a suitable medium such that NOVX protein is produced. In another embodiment, the method further comprises isolating NOVX protein from the medium or the host cell.

Transgenic NOVX Animals

The host cells of the invention can also be used to produce non-human transgenic animals. For example, in one embodiment, a host cell of the invention is a fertilized oocyte or an embryonic stem cell into which NOVX protein-coding sequences have been introduced. Such host cells can then be used to create non-human transgenic animals in which exogenous NOVX sequences have been introduced into their genome or homologous recombinant animals in which endogenous NOVX sequences have been altered. Such animals are useful for studying the function and/or activity of NOVX protein and for identifying and/or evaluating modulators of NOVX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous NOVX gene has been altered by homologous recombination between the endogenous gene and an exogenous DNA molecule introduced into a cell of the animal, e.g., an embryonic cell of the animal, prior to development of the animal.

A transgenic animal of the invention can be created by introducing NOVX-encoding nucleic acid into the male pronuclei of a fertilized oocyte (e.g., by microinjection, retroviral infection) and allowing the oocyte to develop in a pseudopregnant female foster animal. The human NOVX cDNA sequences, i.e., any one of SEQ ID NO: 2n−1, wherein n is an integer between 1 and 124, can be introduced as a transgene into the genome of a non-human animal. Alternatively, a non-human homologue of the human NOVX gene, such as a mouse NOVX gene, can be isolated based on hybridization to the human NOVX CDNA (described further supra) and used as a transgene. Intronic sequences and polyadenylation signals can also be included in the transgene to increase the efficiency of expression of the transgene. A tissue-specific regulatory sequence(s) can be operably-linked to the NOVX transgene to direct expression of NOVX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the NOVX transgene in its genome and/or expression of NOVX mRNA in tissues or cells of the animals. A transgenic founder animal can then be used to breed additional animals carrying the transgene. Moreover, transgenic animals carrying a transgene-encoding NOVX protein can further be bred to other transgenic animals carrying other transgenes.

To create a homologous recombinant animal, a vector is prepared which contains at least a portion of a NOVX gene into which a deletion, addition or substitution has been introduced to thereby alter, e.g., functionally disrupt, the NOVX gene. The NOVX gene can be a human gene (e.g., the cDNA of any one of SEQ ID NO: 2n−1, wherein n is an integer between 1 and 124), but more preferably, is a non-human homologue of a human NOVX gene. For example, a mouse homologue of human NOVX gene of SEQ ID NO: 2n−1, wherein n is an integer between 1 and 124, can be used to construct a homologous recombination vector suitable for altering an endogenous NOVX gene in the mouse genome. In one embodiment, the vector is designed such that, upon homologous recombination, the endogenous NOVX gene is functionally disrupted (i.e., no longer encodes a functional protein; also referred to as a “knock out” vector).

Alternatively, the vector can be designed such that, upon homologous recombination, the endogenous NOVX gene is mutated or otherwise altered but still encodes functional protein (e.g., the upstream regulatory region can be altered to thereby alter the expression of the endogenous NOVX protein). In the homologous recombination vector, the altered portion of the NOVX gene is flanked at its 5′- and 3′-termini by additional nucleic acid of the NOVX gene to allow for homologous recombination to occur between the exogenous NOVX gene carried by the vector and an endogenous NOVX gene in an embryonic stem cell. The additional flanking NOVX nucleic acid is of sufficient length for successful homologous recombination with the endogenous gene. Typically, several kilobases of flanking DNA (both at the 5′- and 3′-termini) are included in the vector. See, e.g., Thomas, et al., 1987. Cell 51: 503 for a description of homologous recombination vectors. The vector is ten introduced into an embryonic stem cell line (e.g., by electroporation) and cells in which the introduced NOVX gene has homologously-recombined with the endogenous NOVX gene are selected. See, e.g., Li, et al., 1992. Cell 69: 915.

The selected cells are then injected into a blastocyst of an animal (e.g., a mouse) to form aggregation chimeras. See, e.g., Bradley, 1987. In: TERATOCARCINOMAS AND EMBRYONIC STEM CELLS: A PRACTICAL APPROACH, Robertson, ed. IRL, Oxford, pp. 113-152. A chimeric embryo can then be implanted into a suitable pseudopregnant female foster animal and the embryo brought to term. Progeny harboring the homologously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol. 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc. Natl. Acad. Sci. USA 89: 6232-6236. Another example of a recombinase system is the FLP recombinase system of Saccharomyces cerevisiae. See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e.g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e.g., a somatic cell) from the transgenic animal can be isolated and induced to exit the growth cycle and enter Go phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

Pharmaceutical Compositions

The NOVX nucleic acid molecules, NOVX proteins, and anti-NOVX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., a NOVX protein or anti-NOVX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an exciplent such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucesal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e.g., U.S. Pat. No. 5,328,470) or by stereotactic injection (see, e.g., Chen, et al., 1994. Proc. Natl. Acad. Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for adminnistration.

Screening and Detection Methods

The isolated nucleic acid molecules of the invention can be used to express NOVX protein (e.g., via a recombinant expression vector in a host cell in gene therapy applications), to detect NOVX mRNA (e.g., in a biological sample) or a genetic lesion in a NOVX gene, and to modulate NOVX activity, as described further, below. In addition, the NOVX proteins can be used to screen drugs or compounds that modulate the NOVX protein activity or expression as well as to treat disorders characterized by insufficient or excessive production of NOVX protein or production of NOVX protein forms that have decreased or aberrant activity compared to NOVX wild-type protein (e.g.; diabetes (regulates insulin release); obesity (binds and transport lipids); metabolic disturbances associated with obesity, the metabolic syndrome X as well as anorexia and wasting disorders associated with chronic diseases and various cancers, and infectious disease(possesses anti-microbial activity) and the various dyslipidemias. In addition, the anti-NOVX antibodies of the invention can be used to detect and isolate NOVX proteins and modulate NOVX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

Screening Assays

The invention provides a method (also referred to herein as a “screening assay”) for identifying modulators, i.e., candidate or test compounds or agents (e.g., peptides, peptidomimetics, small molecules or other drugs) that bind to NOVX proteins or have a stimulatory or inhibitory effect on, e.g., NOVX protein expression or NOVX protein activity. The invention also includes compounds identified in the screening assays described herein.

In one embodiment, the invention provides assays for screening candidate or test compounds which bind to or modulate the activity of the membrane-bound form of a NOVX protein or polypeptide or biologically-active portion thereof. The test compounds of the invention can be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e.g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc. Natl. Acad. Sci. U.S.A. 91: 11422; Zuckermann, et al., 1994. J. Med. Chem. 37: 2678; Cho, et al., 1993. Science 261: 1303; Carrell, et al., 1994. Angew. Chem. Int. Ed. Engl. 33: 2059; Carell, et al., 1994. Angew. Chem. Int. Ed. Engl. 33: 2061; and Gallop, et al., 1994. J. Med. Chem. 37: 1233.

Libraries of compounds may be presented in solution (e.g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl. Acad. Sci. U.S.A. 87: 6378-6382; Felici, 1991. J. Mol. Biol. 222: 301-310; Ladner, U.S. Pat. No. 5,233,409.).

In one embodiment, an assay is a cell-based assay in which a cell which expresses a membrane-bound form of NOVX protein, or a biologically-active portion thereof, on the cell surface is contacted with a test compound and the ability of the test compound to bind to a NOVX protein determined. The cell, for example, can of mammalian origin or a yeast cell. Determining the ability of the test compound to bind to the NOVX protein can be accomplished, for example, by coupling the test compound with a radioisotope or enzymatic label such that binding of the test compound to the NOVX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of NOVX protein, or a biologically-active portion thereof, on the cell surface with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX protein or a biologically-active portion thereof as compared to the known compound.

In another embodiment, an assay is a cell-based assay comprising contacting a cell expressing a membrane-bound form of NOVX protein, or a biologically-active portion thereof, on the cell surface with a test compound and determining the ability of the test compound to modulate (e.g., stimulate or inhibit) the activity of the NOVX protein or biologically-active portion thereof. Determining the ability of the test compound to modulate the activity of NOVX or a biologically-active portion thereof can be accomplished, for example, by determining the ability of the NOVX protein to bind to or interact with a NOVX target molecule. As used herein, a “target molecule” is a molecule with which a NOVX protein binds or interacts in nature, for example, a molecule on the surface of a cell which expresses a NOVX interacting protein, a molecule on the surface of a second cell, a molecule in the extracellular milieu, a molecule associated with the internal surface of a cell membrane or a cytoplasmic molecule. A NOVX target molecule can be a non-NOVX molecule or a NOVX protein or polypeptide of the invention. In one embodiment, a NOVX target molecule is a component of a signal transduction pathway that facilitates transduction of an extracellular signal (e.g. a signal generated by binding of a compound to a membrane-bound NOVX molecule) through the cell membrane and into the cell. The target, for example, can be a second intercellular protein that has catalytic activity or a protein that facilitates the association of downstream signaling molecules with NOVX.

Determining the ability of the NOVX protein to bind to or interact with a NOVX target molecule can be accomplished by one of the methods described above for determining direct binding. In one embodiment, determining the ability of the NOVX protein to bind to or interact with a NOVX target molecule can be accomplished by determining the activity of the target molecule. For example, the activity of the target molecule can be determined by detecting induction of a cellular second messenger of the target (i.e. intracellular Ca²⁺, diacylglycerol, IP3, etc.), detecting catalytic/enzymatic activity of the target an appropriate substrate, detecting the induction of a reporter gene (comprising a NOVX-responsive regulatory element operatively linked to a nucleic acid encoding a detectable marker, e.g., luciferase), or detecting a cellular response, for example, cell survival, cellular differentiation, or cell proliferation.

In yet another embodiment, an assay of the invention is a cell-free assay comprising contacting a NOVX protein or biologically-active portion thereof with a test compound and determining the ability of the test compound to bind to the NOVX protein or biologically-active portion thereof. Binding of the test compound to the NOVX protein can be determined either directly or indirectly as described above. In one such embodiment, the assay comprises contacting the NOVX protein or biologically-active portion thereof with a known compound which binds NOVX to form an assay mixture, contacting the assay mixture with a test compound, and determining the ability of the test compound to interact with a NOVX protein, wherein determining the ability of the test compound to interact with a NOVX protein comprises determining the ability of the test compound to preferentially bind to NOVX or biologically-active portion thereof as compared to the known compound.

In still another embodiment, an assay is a cell-free assay comprising contacting NOVX protein or biologically-active portion thereof with a test compound and determining the ability of the test compound to modulate (e.g. stimulate or inhibit) the activity of the NOVX protein or biologically-active portion thereof. Determining the ability of the test compound to modulate the activity of NOVX can be accomplished, for example, by determining the ability of the NOVX protein to bind to a NOVX target molecule by one of the methods described above for determining direct binding. In an alternative embodiment, determining the ability of the test compound to modulate the activity of NOVX protein can be accomplished by determining the ability of the NOVX protein further modulate a NOVX target molecule. For example, the catalytic/enzymatic activity of the target molecule on an appropriate substrate can be determined as described, supra.

In yet another embodiment, the cell-free assay comprises contacting the NOVX protein or biologically-active portion thereof with a known compound which binds NOVX protein to form an assay mixture, contacting the assay mixture with a test compound, and determining the ability of the test compound to interact with a NOVX protein, wherein determining the ability of the test compound to interact with a NOVX protein comprises determining the ability of the NOVX protein to preferentially bind to or modulate the activity of a NOVX target molecule.

The cell-free assays of the invention are amenable to use of both the soluble form or the membrane-bound form of NOVX protein. In the case of cell-free assays comprising the membrane-bound form of NOVX protein, it may be desirable to utilize a solubilizing agent such that the membrane-bound form of NOVX protein is maintained in solution. Examples of such solubilizing agents include non-ionic detergents such as n-octylglucoside, n-dodecylglucoside, n-dodecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl—N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either NOVX protein or its target molecule to facilitate separation of complexed from uncomplexed forms of one or both of the proteins, as well as to accommodate automation of the assay. Binding of a test compound to NOVX protein, or interaction of NOVX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-NOVX fusion proteins or GST-target fusion proteins can be adsorbed onto glutathione sepharose beads (Sigma Chemical, St. Louis, Mo.) or glutathione derivatized microtiter plates, that are then combined with the test compound or the test compound and either the non-adsorbed target protein or NOVX protein, and the mixture is incubated under conditions conducive to complex formation (e.g., at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of NOVX protein binding or activity determined using standard techniques.

Other techniques for immobilizing proteins on matrices can also be used in the screening assays of the invention. For example, either the NOVX protein or its target molecule can be immobilized utilizing conjugation of biotin and streptavidin. Biotinylated NOVX protein or target molecules can be prepared from biotin-NHS (N-hydroxy-succinimide) using techniques well-known within the art (e.g., biotinylation kit, Pierce Chernicals, Rockford, Ill.), and immobilized in the wells of streptavidin-coated 96 well plates (Pierce Chemical). Alternatively, antibodies reactive with NOVX protein or target molecules, but which do not interfere with binding of the NOVX protein to its target molecule, can be derivatized to the wells of the plate, and unbound target or NOVX protein trapped in the wells by antibody conjugation. Methods for detecting such complexes, in addition to those described above for the GST-immobilized complexes, include immunodetection of complexes using antibodies reactive with the NOVX protein or target molecule, as well as enzyme-linked assays that rely on detecting an enzymatic activity associated with the NOVX protein or target molecule.

In another embodiment, modulators of NOVX protein expression are identified in a method wherein a cell is contacted with a candidate compound and the expression of NOVX mRNA or protein in the cell is determined. The level of expression of NOVX mRNA or protein in the presence of the candidate compound is compared to the level of expression of NOVX mRNA or protein in the absence of the candidate compound. The candidate compound can then be identified as a modulator of NOVX mRNA or protein expression based upon this comparison. For example, when expression of NOVX mRNA or protein is greater (i.e., statistically significantly greater) in the presence of the candidate compound than in its absence, the candidate compound is identified as a stimulator of NOVX mRNA or protein expression. Alternatively, when expression of NOVX mRNA or protein is less (statistically significantly less) in the presence of the candidate compound than in its absence, the candidate compound is identified as an inhibitor of NOVX mRNA or protein expression. The level of NOVX mRNA or protein expression in the cells can be determined by methods described herein for detecting NOVX mRNA or protein.

In yet another aspect of the invention, the NOVX proteins can be used as “bait proteins” in a two-hybrid assay or three hybrid assay (see, e.g., U.S. Pat. No. 5,283,317; Zervos, et al., 1993. Cell 72: 223-232; Madura, et al., 1993. J. Biol. Chem. 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with NOVX (“NOVX-binding proteins” or “NOVX-bp”) and modulate NOVX activity. Such NOVX-binding proteins are also involved in the propagation of signals by the NOVX proteins as, for example, upstream or downstream elements of the NOVX pathway.

The two-hybrid system is based on the modular nature of most transcription factors, which consist of separable DNA-binding and activation domains. Briefly, the assay utilizes two different DNA constructs. In one construct, the gene that codes for NOVX is fused to a gene encoding the DNA binding domain of a known transcription factor (e.g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming a NOVX-dependent complex, the DNA-binding and activation domains of the transcription factor are brought into close proximity. This proximity allows transcription of a reporter gene (e.g., LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with NOVX.

The invention further pertains to novel agents identified by the aforementioned screening assays and uses thereof for treatments as described herein.

Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

Chromosome Mapping

Once the sequence (or a portion of the sequence) of a gene has been isolated, this sequence can be used to map the location of the gene on a chromosome. This process is called chromosome mapping. Accordingly, portions or fragments of the NOVX sequences of SEQ ID NO: 2n−1, wherein n is an integer between 1 and 124, or fragments or derivatives thereof, can be used to map the location of the NOVX genes, respectively, on a chromosome. The mapping of the NOVX sequences to chromosomes is an important first step in correlating these sequences with genes associated with disease.

Briefly, NOVX genes can be mapped to chromosomes by preparing PCR primers (preferably 15-25 bp in length) from the NOVX sequences. Computer analysis of the NOVX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the NOVX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1983. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the NOVX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e.g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the NOVX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

Tissue Typing

The NOVX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the NOVX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The NOVX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If coding sequences, such as those of SEQ ID NO: 2n−1, wherein n is an integer between 1 and 124, are used, a more appropriate number of primers for positive individual identification would be 500-2,000.

Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly, one aspect of the invention relates to diagnostic assays for determining NOVX protein and/or nucleic acid expression as well as NOVX activity, in the context of a biological sample (e.g., blood, serum, cells, tissue) to thereby determine whether an individual is afflicted with a disease or disorder, or is at risk of developing a disorder, associated with aberrant NOVX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with NOVX protein, nucleic acid expression or activity. For example, mutations in a NOVX gene can be assayed in a biological sample. Such assays can be used for prognostic or predictive purpose to thereby prophylactically treat an individual prior to the onset of a disorder characterized by or associated with NOVX protein, nucleic acid expression, or biological activity.

Another aspect of the invention provides methods for determining NOVX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g., the genotype of the individual examined to determine the ability of the individual to respond to a particular agent.)

Yet another aspect of the invention pertains to monitoring the influence of agents (e.g., drugs, compounds) on the expression or activity of NOVX in clinical trials.

These and other agents are described in further detail in the following sections.

Diagnostic Assays

An exemplary method for detecting the presence of absence of NOVX in a biological sample involves obtaining a biological sample from a test subject and contacting the biological sample with a compound or an agent capable of detecting NOVX protein or nucleic acid (e.g., mRNA, genomic DNA) that encodes NOVX protein such that the presence of NOVX is detected in the biological sample. An agent for detecting NOVX mRNA or genomic DNA is a labeled nucleic acid probe capable of hybridizing to NOVX mRNA or genomic DNA. The nucleic acid probe can be, for example, a full-length NOVX nucleic acid, such as the nucleic acid of SEQ ID NO: 2n−1, wherein n is an integer between 1 and 124, or a portion thereof, such as an oligonucleotide of at least 15, 30, 50, 100, 250 or 500 nucleotides in length and sufficient to specifically hybridize under stringent conditions to NOVX mRNA or genomic DNA. Other suitable probes for use in the diagnostic assays of the invention are described herein.

An agent for detecting NOVX protein is an antibody capable of binding to NOVX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e., physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect NOVX mRNA, protein, or genomic DNA in a biological sample in vitro as well as in vivo. For example, in vitro techniques for detection of NOVX mRNA include Northern hybridizations and in situ hybridizations. In vitro techniques for detection of NOVX protein include enzyme linked immunosorbent assays (ELISAs), Western blots, immunoprecipitations, and immunofluorescence. In vitro techniques for detection of NOVX genomic DNA include Southern hybridizations. Furthermore, in vivo techniques for detection of NOVX protein include introducing into a subject a labeled anti-NOVX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting NOVX protein, mRNA, or genomic DNA, such that the presence of NOVX protein, mRNA or genomic DNA is detected in the biological sample, and comparing the presence of NOVX protein, mRNA or genomic DNA in the control sample with the presence of NOVX protein, mRNA or genomic DNA in the test sample.

The invention also encompasses kits for detecting the presence of NOVX in a biological sample. For example, the kit can comprise: a labeled compound or agent capable of detecting NOVX protein or mRNA in a biological sample; means for determining the amount of NOVX in the sample; and means for comparing the amount of NOVX in the sample with a standard. The compound or agent can be packaged in a suitable container. The kit can further comprise instructions for using the kit to detect NOVX protein or nucleic acid.

Prognostic Assays

The diagnostic methods described herein can furthermore be utilized to identify subjects having or at risk of developing a disease or disorder associated with aberrant NOVX expression or activity. For example, the assays described herein, such as the preceding diagnostic assays or the following assays, can be utilized to identify a subject having or at risk of developing a disorder associated with NOVX protein, nucleic acid expression or activity. Alternatively, the prognostic assays can be utilized to identify a subject having or at risk for developing a disease or disorder. Thus, the invention provides a method for identifying a disease or disorder associated with aberrant NOVX expression or activity in which a test sample is obtained from a subject and NOVX protein or nucleic acid (e.g., mRNA, genomic DNA) is detected, wherein the presence of NOVX protein or nucleic acid is diagnostic for a subject having or at risk of developing a disease or disorder associated with aberrant NOVX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e.g., an agonist, antagonist, peptidomimetic, protein, peptide, nucleic acid, small molecule, or other drug candidate) to treat a disease or disorder associated with aberrant NOVX expression or activity. For example, such methods can be used to determine whether a subject can be effectively treated with an agent for a disorder. Thus, the invention provides methods for determining whether a subject can be effectively treated with an agent for a disorder associated with aberrant NOVX expression or activity in which a test sample is obtained and NOVX protein or nucleic acid is detected (e.g., wherein the presence of NOVX protein or nucleic acid is diagnostic for a subject that can be administered the agent to treat a disorder associated with aberrant NOVX expression or activity).

The methods of the invention can also be used to detect genetic lesions in a NOVX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding a NOVX-protein, or the misexpression of the NOVX gene. For example, such genetic lesions can be detected by ascertaining the existence of at least one of: (i) a deletion of one or more nucleotides from a NOVX gene; (ii) an addition of one or more nucleotides to a NOVX gene; (iii) a substitution of one or more nucleotides of a NOVX gene, (iv) a chromosomal rearrangement of a NOVX gene; (v) an alteration in the level of a messenger RNA transcript of a NOVX gene, (vi) aberrant modification of a NOVX gene, such as of the methylation pattern of the genomic DNA, (vii) the presence of a non-wild-type splicing pattern of a messenger RNA transcript of a NOVX gene, (viii) a non-wild-type level of a NOVX protein, (ix) allelic loss of a NOVX gene, and (x) inappropriate post-translational modification of a NOVX protein. As described herein, there are a large number of assay techniques known in the art which can be used for detecting lesions in a NOVX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g., U.S. Pat. Nos. 4,683,195 and 4,683,202), such as anchor PCR or RACE PCR, or, alternatively, in a ligation chain reaction (LCR) (see, e.g., Landegran, et al., 1988. Science 241: 1077-1080; and Nakazawa, et al., 1994. Proc. Natl. Acad. Sci. USA 91: 360-364), the latter of which can be particularly useful for detecting point mutations in the NOVX-gene (see, Abravaya, et al., 1995. Nucl. Acids Res. 23: 675-682). This method can include the steps of collecting a sample of cells from a patient, isolating nucleic acid (e.g., genomic, mRNA or both) from the cells of the sample, contacting the nucleic acid sample with one or more primers that specifically hybridize to a NOVX gene under conditions such that hybridization and amplification of the NOVX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustained sequence replication (see, Guatelli, et al., 1990. Proc. Natl. Acad. Sci. USA 87: 1874-1878), transcriptional amplification system (see, Kwoh, et al., 1989. Proc. Natl. Acad. Sci. USA 86: 1173-1177); Qβ Replicase (see, Lizardi, et al, 1988. BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in a NOVX gene from a sample cell can be identified by alterations in restriction enzyme cleavage patterns. For example, sample and control DNA is isolated, amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e.g., U.S. Pat. No. 5,493,531) can be used to score for the presence of specific mutations by development or loss of a ribozyme cleavage site.

In other embodiments, genetic mutations in NOVX can be identified by hybridizing a sample and control nucleic acids, e.g., DNA or RNA, to high-density arrays containing hundreds or thousands of oligonucleotides probes. See, e.g., Cronin, et al., 1996. Human Mutation 7: 244-255; Kozal, et al., 1996. Nat. Med. 2: 753-759. For example, genetic mutations in NOVX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the NOVX gene and detect mutations by comparing the sequence of the sample NOVX with the corresponding wild-type (control) sequence. Examples of sequencing reactions include those based on techniques developed by Maxim and Gilbert, 1977. Proc. Natl. Acad. Sci. USA 74: 560 or Sanger, 1977. Proc. Natl. Acad. Sci. USA 74: 5463. It is also contemplated that any of a variety of automated sequencing procedures can be utilized when performing the diagnostic assays (see, e.g., Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol. 38: 147-159).

Other methods for detecting mutations in the NOVX gene include methods in which protection from cleavage agents is used to detect mismatched bases in RNA/RNA or RNA/DNA heteroduplexes. See, e.g., Myers, et al., 1985. Science 230: 1242. In general, the art technique of “mismatch cleavage” starts by providing heteroduplexes of formed by hybridizing (labeled) RNA or DNA containing the wild-type NOVX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated waith hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc. Natl. Acad. Sci. USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in NOVX cDNAs obtained from samples of cells. For example, the mutY enzyme of E. coli cleaves A at G/A mismatches and the thymidine DNA glycosylase from HeLa cells cleaves T at G/T mismatches. See, e.g., Hsu, et al., 1994. Carcinogenesis 15: 1657-1662. According to an exemplary embodiment, a probe based on a NOVX sequence, e.g., a wild-type NOVX sequence, is hybridized to a cDNA or other DNA product from a test cell(s). The duplex is treated with a DNA mismatch repair enzyme, and the cleavage products, if any, can be detected from electrophoresis protocols or the like. See, e.g., U.S. Pat. No. 5,459,039.

In other embodiments, alterations in electrophoretic mobility will be used to identify mutations in NOVX genes. For example, single strand conformation polymorphism (SSCP) may be used to detect differences in electrophoretic mobility between mutant and wild type nucleic acids. See, e.g., Orita, et al., 1989. Proc. Natl. Acad. Sci. USA: 86: 2766; Cotton, 1993. Mutat. Res. 285: 125-144; Hayashi, 1992. Genet. Anal. Tech. Appl. 9: 73-79. Single-stranded DNA fragments of sample and control NOVX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g., Keen, et al., 1991. Trends Genet. 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g., Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc. Natl. Acad. Sci. USA 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e.g., Gasparini, et al., 1992. Mol. Cell Probes 6: 1. It is anticipated that in certain embodiments amplification may also be performed using Taq ligase for amplification. See, e.g., Barany, 1991. Proc. Natl. Acad. Sci. USA 88: 189. In such cases, ligation will occur only if there is a perfect match at the 3′-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving a NOVX gene.

Furthermore, any cell type or tissue, preferably peripheral blood leukocytes, in which NOVX is expressed may be utilized in the prognostic assays described herein. However, any biological sample containing nucleated cells may be used, including, for example, buccal mucosal cells.

Pharmacogenomics

Agents, or modulators that have a stimulatory or inhibitory effect on NOVX activity (e.g., NOVX gene expression), as identified by a screening assay described herein can be administered to individuals to treat (prophylactically or therapeutically) disorders. The disorders include but are not limited to, e.g., those diseases, disorders and conditions listed above, and more particularly include those diseases, disorders, or conditions associated with homologs of a NOVX protein, such as those summarized in Table A.

In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g., drugs) for prophylactic or therapeutic treatments based on a consideration of the individual's genotype. Such pharmacogenomics can further be used to determine appropriate dosages and therapeutic regimens. Accordingly, the activity of NOVX protein, expression of NOVX nucleic acid, or mutation content of NOVX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e.g., Eichelbaum, 1996. Clin. Exp. Pharmacol. Physiol., 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofurans) and consumption of fava beans.

As an illustrative embodiment, the activity of drug metabolizing enzymes is a major determinant of both the intensity and duration of drug action. The discovery of genetic polymorphisms of drug metabolizing enzymes (e.g., N-acetyltransferase 2 (NAT 2) and cytochrome pregnancy zone protein precursor enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the population, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of NOVX protein, expression of NOVX nucleic acid, or mutation content of NOVX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with a NOVX modulator, such as a modulator identified by one of the exemplary screening assays described herein.

Monitoring of Effects During Clinical Trials

Monitoring the influence of agents (e.g., drugs, compounds) on the expression or activity of NOVX (e.g., the ability to modulate aberrant cell proliferation and/or differentiation) can be applied not only in basic drug screening, but also in clinical trials. For example, the effectiveness of an agent determined by a screening assay as described herein to increase NOVX gene expression, protein levels, or upregulate NOVX activity, can be monitored in clinical trails of subjects exhibiting decreased NOVX gene expression, protein levels, or downregulated NOVX activity. Alternatively, the effectiveness of an agent determined by a screening assay to decrease NOVX gene expression, protein levels, or downregulate NOVX activity, can be monitored in clinical trails of subjects exhibiting increased NOVX gene expression, protein levels, or upregulated NOVX activity. In such clinical trials, the expression or activity of NOVX and, preferably, other genes that have been implicated in, for example, a cellular proliferation or immune disorder can be used as a “read out” or markers of the immune responsiveness of a particular cell.

By way of example, and not of limitation, genes, including NOVX, that are modulated in cells by treatment with an agent (e.g., compound, drug or small molecule) that modulates NOVX activity (e.g., identified in a screening assay as described herein) can be identified. Thus, to study the effect of agents on cellular proliferation disorders, for example, in a clinical trial, cells can be isolated and RNA prepared and analyzed for the levels of expression of NOVX and other genes implicated in the disorder. The levels of gene expression (i.e., a gene expression pattern) can be quantified by Northern blot analysis or RT-PCR, as described herein, or alternatively by measuring the amount of protein produced, by one of the methods as described herein, or by measuring the levels of activity of NOVX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g., an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of a NOVX protein, mRNA, or genomic DNA in the preadministration sample; (iii) obtaining one or more post-administration samples from the subject; (iv) detecting the level of expression or activity of the NOVX protein, mRNA, or genomic DNA in the post-administration samples; (v) comparing the level of expression or activity of the NOVX protein, mRNA, or genomic DNA in the pre-administration sample with the NOVX protein, mRNA, or genomic DNA in the post administration sample or samples; and (vi) altering the administration of the agent to the subject accordingly. For example, increased administration of the agent may be desirable to increase the expression or activity of NOVX to higher levels than detected, i.e., to increase the effectiveness of the agent. Alternatively, decreased administration of the agent may be desirable to decrease expression or activity of NOVX to lower levels than detected, i.e., to decrease the effectiveness of the agent.

Methods of Treatment

The invention provides for both prophylactic and therapeutic methods of treating a subject at risk of (or susceptible to) a disorder or having a disorder associated with aberrant NOVX expression or activity. The disorders include but are not limited to, e.g., those diseases, disorders and conditions listed above, and more particularly include those diseases, disorders, or conditions associated with homologs of a NOVX protein, such as those summarized in Table A.

These methods of treatment will be discussed more fully, below.

Diseases and Disorders

Diseases and disorders that are characterized by increased (relative to a subject not suffering from the disease or disorder) levels or biological activity may be treated with Therapeutics that antagonize (i.e., reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244: 1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Prophylactic Methods

In one aspect, the invention provides a method for preventing, in a subject, a disease or condition associated with an aberrant NOVX expression or activity, by administering to the subject an agent that modulates NOVX expression or at least one NOVX activity. Subjects at risk for a disease that is caused or contributed to by aberrant NOVX expression or activity can be identified by, for example, any or a combination of diagnostic or prognostic assays as described herein. Administration of a prophylactic agent can occur prior to the manifestation of symptoms characteristic of the NOVX aberrancy, such that a disease or disorder is prevented or, alternatively, delayed in its progression. Depending upon the type of NOVX aberrancy, for example, a NOVX agonist or NOVX antagonist agent can be used for treating the subject. The appropriate agent can be determined based on screening assays described herein. The prophylactic methods of the invention are further discussed in the following subsections.

Therapeutic Methods

Another aspect of the invention pertains to methods of modulating NOVX expression or activity for therapeutic purposes. The modulatory method of the invention involves contacting a cell with an agent that modulates one or more of the activities of NOVX protein activity associated with the cell. An agent that modulates NOVX protein activity can be an agent as described herein, such as a nucleic acid or a protein, a naturally-occurring cognate ligand of a NOVX protein, a peptide, a NOVX peptidomimetic, or other small molecule. In one embodiment, the agent stimulates one or more NOVX protein activity. Examples of such stimulatory agents include active NOVX protein and a nucleic acid molecule encoding NOVX that has been introduced into the cell. In another embodiment, the agent inhibits one or more NOVX protein activity. Examples of such inhibitory agents include antisense NOVX nucleic acid molecules and anti-NOVX antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder characterized by aberrant expression or activity of a NOVX protein or nucleic acid molecule. In one embodiment, the method involves administering an agent (e.g., an agent identified by a screening assay described herein), or combination of agents that modulates (e.g., up-regulates or down-regulates) NOVX expression or activity. In another embodiment, the method involves administering a NOVX protein or nucleic acid molecule as therapy to compensate for reduced or aberrant NOVX expression or activity.

Stimulation of NOVX activity is desirable in situations in which NOVX is abnormally downregulated and/or in which increased NOVX activity is likely to have a beneficial effect. One example of such a situation is where a subject has a disorder characterized by aberrant cell proliferation and/or differentiation (e.g., cancer or immune associated disorders). Another example of such a situation is where the subject has a gestational disease (e.g., preclampsia).

Determination of the Biological Effect of the Therapeutic

In various embodiments of the invention, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Prophylactic and Therapeutic Uses of the Compositions of the Invention

The NOVX nucleic acids and proteins of the invention are useful in potential prophylactic and therapeutic applications implicated in a variety of disorders. The disorders include but are not limited to, e.g., those diseases, disorders and conditions listed above, and more particularly include those diseases, disorders, or conditions associated with homologs of a NOVX protein, such as those summarized in Table A.

As an example, a cDNA encoding the NOVX protein of the invention may be useful in gene therapy, and the protein may be useful when administered to a subject in need thereof. By way of non-limiting example, the compositions of the invention will have efficacy for treatment of patients suffering from diseases, disorders, conditions and the like, including but not limited to those listed herein.

Both the novel nucleic acid encoding the NOVX protein, and the NOVX protein of the invention, or fragments thereof, may also be useful in diagnostic applications, wherein the presence or amount of the nucleic acid or the protein are to be assessed. A further use could be as an anti-bacterial molecule (i.e., some peptides have been found to possess anti-bacterial properties). These materials are further useful in the generation of antibodies, which immunospecifically-bind to the novel substances of the invention for use in therapeutic or diagnostic methods.

The invention will be further described in the following examples, which do not limit the scope of the invention described in the claims.

### EXAMPLES

### Example A: Polynucleotide and Polypeptide Sequences, and Homology Data

### Example 1

The NOV1 clone was analyzed, and the nucleotide and encoded polypeptide sequences are shown in Table 1A.

TABLE 1A
NOV1 Sequence Analysis

| | |
|---|---|
| SEQ ID NO: 1 | 6189 bp |

| | |
|---|---|
| NOV1a, | ATGTTGAAGTTCAAATATGGAGCGCGGAATCCTTTGGATGCTGGTGCTGCTGAACCCATTGCCAGCCG |
| CG106764-01 | |
| DNA Sequence | GGCCTCCAGGCTGAATCTGTTCTTCCAGGGGAAACCACCCTTTATGACTCAACAGCAGATGTCTCCTC |
| | TTTCCCGAGAAGGGATATTAGATGCCCTCTTTGTTCTCTTTGAAGAATGCAGTCAGCCTGCTCTGATG |
| | AAGATTAAGCACGTGAGCAACTTTGTCCGGAAGTGTTCCGACACCATAGCTGAGTTACAGGAGCTCCA |
| | GCCTTCGGCAAAGGACTTCGAAGTCAGAAGTCTTGTAGGTTGTGGTCACTTTGCTGAAGTGCAGGTGG |
| | TAAGAGAGAAAGCAACCGGGGACATCTATGCTATGAAAGTGATGAAGAAGAAGGCTTTATTGGCCCAG |
| | GAGCAGGTTTCATTTTTTGAGGAAGAGCGGAACATATTATCTCGAAGCACAAGCCCGTGGATCCCCCA |
| | ATTACAGTATGCCTTTCAGGACAAAAATCACCTTTATCTGGTGATGGAATATCAGCCTGGAGGGGACT |
| | TGCTGTCACTTTTGAATAGATATGAGGACCAGTTAGATGAAAACCTGATACAGTTTTACCTAGCTGAG |
| | CTGATTTTGGCTGTTCACAGCGTTCATCTGATGGGATACGTGCATCGGGACATCAAGCCTGAGAACAT |
| | TCTCGTTGACCGCACAGGACACATCAAGCTGGTGGATTTTGGATCTGCCGCGAAAATGAATTCAAACA |
| | AGGTGAATGCCAAACTCCCGATTGGGACCCCAGATTACATGGCTCCTGAAGTGCTGACTGTGATGAAC |
| | GGGGATGGAAAAGGCACCTACGGCCTGGACTGTGACTGGTGGTCAGTGGGCGTGATTGCCTATGAGAT |
| | GATTTATGGGAGATCCCCCTTCGCAGAGGGAACCTCTGCCAGAACCTTCAATAACATTATGAATTTCC |
| | AGCGGTTTTTGAAATTTCCAGATGACCCCAAAGTGAGCAGTGACTTTCTTGATCTGATTCAAAGCTTG |
| | TTGTGCGGCCAGAAAGAGAGACTGAAGTTTGAAGGTCTTTGCTGCCATCCTTTCTTCTCTAAAATTGA |
| | CTGGAACAACATTCGTAACGCTCCTCCCCCCTTCGTTCCCACCCTCAAGTCTGACGATGACACCTCCA |
| | ATTTTGATGAACCAGAGAAGAATTCGTGGGTTTCATCCTCTCCGTGCCAGCTGAGCCCCTCAGGCTTC |
| | TCGGGTGAAGAACTGCCGTTTGTGGGGTTTTCGTACAGCAAGGCACTGGGGATTCTTGGTAGATCTGA |
| | GTCTGTTGTGTCGGGTCTGGACTCCCCTGCCAAGACTAGCTCCATGGAAAAGAAACTTCTCATCAAAA |
| | GCAAAGAGCTACAAGACTCTCAGGACAAGTGTCACAAGATGGAGCAGGAAATGACCCGGTTACATCGG |
| | AGAGTGTCAGAGGTGGAGGCTGTGCTTAGTCAGAAGGAGGTGGAGCTGAAGGCCTCTGAGACTCAGAG |
| | ATCCCTCCTGGAGCAGGACCTTGCTACCTACATCACAGAATGCAGTAGCTTAAAGCGAAGTTTGGAGC |
| | AAGCACGGATGGAGGTGTCCCAGGAGGATGACAAAGCACTGCAGCTTCTCCATGATATCAGAGAGCAG |
| | AGCCGGAAGCTCCAAGAAATCAAAGAGCAGGAGTACCAGGCTCAAGTGGAAGAAATGAGGTTGATGAT |
| | GAATCAGTTGGAAGAGGATCTTGTCTCAGCAAGAAGACGGAGTGATCTCTACGAATCTGAGCTGAGAG |
| | AGTCTCGGCTTGCTGCTGAAGAATTCAAGCGGAAAGCGACAGAATGTCAGCATAAACTGTTGAAGGCT |
| | AAGGATCAGGGGAAGCCTGAAGTGGGAGAATATGCGAAACTGGAGAAGATCAATGCTGAGCAGCAGCT |
| | CAAAATTCAGGAGCTCCAAGAGAAACTGGAGAAGGCTGTAAAAGCCAGCACGGAGGCCACCGAGCTGC |
| | TGCAGAATATCCGCCAGGCAAAGGAGCGAGCCGAGAGGGAGCTGGAGAAGCTGCAGAACCGAGAGGAT |
| | TCTTCTGAAGGCATCAGAAAGAAGCTGGTGGAAGCTGAGGAACGCCGCCATTCTCTGGAGAACAAGGT |
| | AAAGAGACTAGAGACCATGGAGCGTAGAGAAAACAGACTGAAGGATGACATCCAGACAAAATCCCAAC |
| | AGATCCAGCAGATGGCTGATAAAATTCTGGAGCTCGAAGAGAAACATCGGGAGGCCCAAGTCTCAGCC |
| | CAGCACCTAGAAGTGCACCTGAAACAGAAAGAGCAGCACTATGAGGAAAAGATTAAAGTATTGGACAA |
| | TCAGATAAAGAAAGACCTGGCTGACAAGGAGACACTGGAGAACATGATGCAGAGACACGAGGAGGAGG |
| | CCCATGAGAAGGGCAAAATTCTCAGCGAACAGAAGGCGATGATCAATGCTATGGATTCCAAGATCAGA |
| | TCCCTGGAACAGAGGATTGTGGAACTGTCTGAAGCCAATAAACTTGCAGCAAATAGCAGTCTTTTTAC |
| | CCAAAGGAACATGAAGGCCCAAGAAGAGATGATTTCTGAACTCAGGCAACAGAAATTTTACCTGGAGA |
| | CACAGGCTGGGAAGTTGGAGGCCCAGAACCGAAAACTGGAGGAGCAGCTGGAGAAGATCAGCCACCAA |
| | GACCACAGTGACAAGAATCGGCTGCTGGAACTGGAGACAAGATTGCGGGAGGTGAGTCTAGAGCACGA |
| | GGAGCAGAAACTGGAGCTCAAGCGCCAGCTCACAGAGCTACAGCTCTCCCTGCAGGAGCGCGAGTCAC |
| | AGTTGACAGCCCTGCAGGCTGCACGGGCGGCCCTGGAGAGCCAGCTTCGCCAGGCGAAGACAGAGCTG |
| | GAAGAGACCACAGCAGAAGCTGAAGAGGAGATCCAGGCACTCACGGCACATAGAGATGAAATCCAGCG |
| | CAAATTTGATGCTCTTCGTAACAGCTGTACTGTGATCACAGACCTGGAGGAGCAGCTAAACCAGCTGA |
| | CCGAGGACAACGCTGAACTCAACAACCAAAACTTCTACTTGTCCAAACAACTCGATGAGGCTTCTGGC |
| | GCCAACGACGAGATTGTACAACTGCGAAGTGAAGTGGACCATCTCCGCCGGGAGATCACGGAACGAGA |
| | GATGCAGCTTACCAGCCAGAAGCAAACGATGGAGGCTCTGAAGACCACGTGCACCATGCTGGAGGAAC |
| | AGGTCATGGATTTGGAGGCCCTAAACGATGAGCTGCTAGAAAAAGAGCGGCAGTGGGAGGCCTGGAGG |
| | AGCGTCCTGGGTGATGAGAAATCCCAGTTTGAGTGTCGGGTTCGAGAGCTGCAGAGGATGCTGGACAC |
| | CGAGAAACAGAGCAGGGCGAGAGCCGATCAGCGGATCACCGAGTCTCGCCAGGTGGTGGAGCTGGCAG |
| | TGAAGGAGCACAAGGCTGAGATTCTCGCTCTGCAGCAGGCTCTCAAAGAGCAGAAGCTGAAGGCCGAG |
| | AGCCTCTCTGACAAGCTCAATGACCTGGAGAAGAAGCATGCTATGCTTGAAATGAATGCCCGAAGCTT |
| | ACAGCAGAAGCTGGAGACTGAACGAGAGCTCAAACAGAGGCTTCTGGAAGAGCAAGCCAAATTACAGC |
| | AGCAGATGGACCTGCAGAAAAATCACATTTTCCGTCTGACTCAAGGACTGCAAGAAGCTCTAGATCGG |
| | GCTGATCTACTGAAGACAGAAAGAAGTGACTTGGAGTATCAGCTGGAAAACATTCAGGTGCTCTATTC |
| | TCATGAAAAGGTGAAAATGGAAGGCACTATTTCTCAACAAACCAAACTCATTGATTTTCTGCAAGCCA |
| | AAATGGACCAACCTGCTAAAAAGAAAAAGGTGCCTCTGCAGTACAATGAGCTGAAGCTGGCCCTGGAG |
| | AAGGAGAAAGCTCGCTGTGCAGAGCTAGAGGAAGCCCTTCAGAAGACCCGCATCGAGCTCCGGTCCGC |
| | CCGGGAGGAAGCTGCCCACCGCAAAGCAACGGACCACCCACACCCATCCACGCCAGCCACCGCGAGGC |
| | AGCAGATCGCCATGTCTGCCATCGTGCGGTCGCCAGAGCACCAGCCCAGTGCCATGAGCCTGCTGGCC |
| | CCGCCATCCAGCCGCAGAAAGGAGTCTTCAACTCCAGAGGAATTTAGTCGGCGTCTTAAGGAACGCAT |
| | GCACCACAATATTCCTCACCGATTCAACGTAGGACTGAACATGCGAGCCACAAAGTGTGCTGTGTGTC |
| | TGGATACCGTGCACTTTGGACGCCAGGCATCCAAATGTCTAGAATGTCAGGTGATGTGTCACCCCAAG |
| | TGCTCCACGTGCTTGCCAGCCACCTGCGGCTTGCCTGCTGAATATGCCACACACTTCACCGAGGCCTT |
| | CTGCCGTGACAAAATGAACTCCCCAGGTCTCCAGACCAAGGAGCCCAGCAGCAGCTTGCACCTGGAAG |
| | GGTGGATGAAGGTGCCCAGGAATAACAAACGAGGACAGCAAGGCTGGGACAGGAAGTACATTGTCCTG |
| | GAGGGATCAAAAGTCCTCATTTATGACAATGAAGCCAGAGAAGCTGGACAGAGGCCGGTGGAAGAATT |
| | TGAGCTGTGCCTTCCCGACGGGGATGTATCTATTCATGGTGCCGTTGGTGCTTCCGAACTCGCAAATA |
| | CAGCCAAAGCAGATGTCCCATACATACTGAAGATGGAATCTCACCCGCACACCACCTGCTGGCCCGGG |
| | AGAACCCTCTACTTGCTAGCTCCCAGCTTCCCTGACAAACAGCGCTGGGTCACCGCCTTAGAATCAGT |
| | TGTCGCAGGTGGGAGAGTTTCTAGGGAAAAAGCAGAAGCTGATGCTAAACTGCTTGGAAACTCCCTGC |
| | TGAAACTGGAAGGTGATGACCGTCTAGACATGAACTGCACGCTGCCCTTCAGTGACCAGGTAGTGTTG |
| | GTGGGCACCGAGGAAGGGCTCTACGCCCTGAATGTCTTGAAAAACTCCCTAACCCATGTCCCAGGAAT |
| | TGGAGCAGTCTTCCAAATTTATATTATCAAGGACCTGGAGAAGCTACTCATGATAGCAGGTGAAGAGC |
| | GGGCACTGTGTCTTGTGGACGTGAAGAAAGTGAAACAGTCCCTGGCCCAGTCCCACCTGCCTGCCCAG |
| | CCCGACATCTCACCCAACATTTTTGAAGCTGTCAAGGGCTGCCACTTGTTTGGGGCAGGCAAGATTGA |
| | GAACGGGCTCTGCATCTGTGCAGCCATGCCCAGCAAAGTCGTCATTCTCCGCTACAACGAAAACCTCA |
| | GCAAATACTGCATCCGGAAAGAGATAGAGACCTCAGAGCCCTGCAGCTGTATCCACTTCACCAATTAC |
| | AGTATCCTCATTGGAACCAATAAATTCTACGAAATCGACATGAAGCAGTACACGCTCGAGGAATTCCT |
| | GGATAAGAATGACCATTCCTTGGCACCTGCTGTGTTTGCCGCCTCTTCCAACAGCTTCCCTGTCTCAA |
| | TCGTGCAGGTGAACAGCGCAGGGCAGCGAGAGGAGTACTTGCTGTGTTTCCACGAATTTGGAGTGTTC |
| | GTGGATTCTTACGGAAGACGTAGCCGCACAGACGATCTCAAGTGGAGTCGCTTACCTTTGGCCTTTGC |
| | CTACAGAGAACCCTATCTGTTTGTGACCCACTTCAACTCACTCGAAGTAATTGAGATCCAGGCACGCT |
| | CCTCAGCAGGGACCCCTGCCCGAGCGTACCTGGACATCCCGAACCCGCGCTACCTGGGGCCTGCCATT |
| | TCCTCAGGAGCGATTTACTTGGCGTCCTCATACCAGGATAAATTAAGGGTCATTTGCTGCAAGGGAAA |
| | CCTCGTGAAGGAGTCCGGCACTGAACACCACCGGGGCCCGTCCACCTCCCGCAGCAGCCCCAACAAGC |
| | GAGGCCCACCCACGTACAACGAGCACATCACCAAGCGCGTGGCCTCCAGCCCAGCGCCGCCCGAAGGC |
| | CCCAGCCACCCGCGAGAGCCAAGCACACCCCACCGCTACCGCGAGGGGCGGACCGAGCTGCGCAGGGA |
| | CAAGTCTCCTGGCCGCCCCCTGGAGCGAGAGAAGTCCCCCGGCCGGATGCTCAGCACGCGGAGAGAGC |
| | GGTCCCCCGGGAGGCTGTTTGAAGACAGCAGCAGGGGCCGGCTGCCTGCGGGAGCCGTGAGGACCCCG |
| | CTGTCCCAGGTGAACAAGGTGTGGGACCAGTCTTCAGTATAAATCTCAGCCAGAAAAACCAACTCCTC |
| | A |

| | | |
|---|---|---|
| ORF Start: ATG at 1 | | ORF Stop: TAA at 6160 |
| SEQ ID NO: 2 | 2053 aa | MW at 234700.1 kD |

| | |
|---|---|
| NOV1a, | MLKFKYGARNPLDAGAAEPIASRASRLNLFFQGKPPFMTQQQMSPLSREGILDALFVLFEECSQPALM |
| CG106764-01 | |
| Protein | KIKHVSNFVRKCSDTIAELQELQPSAKDFEVRSLVGCGHFAEVQVVREKATGDIYAMKVMKKKALLAQ |
| Sequence | |
| | EQVSFFEEERNILSRSTSPWIPQLQYAFQDKNHLYLVMEYQPGGDLLSLLNRYEDQLDENLIQFYLAE |
| | LILAVHSVHLMGYVHRDIKPENILVDRTGHIKLVDFGSAAKMNSNKVNAKLPIGTPDYMAPEVLTVMN |
| | GDGKGTYGLDCDWWSVGVIAYEMIYGRSPFAEGTSARTFNNIMNFQRFLKFPDDPKVSSDFLDLIQSL |
| | LCGQKERLKFEGLCCHPFFSKIDWNNIRNAPPPFVPTLKSDDDTSNFDEPEKNSWVSSSPCQLSPSGF |
| | SGEELPFVGFSYSKALGILGRSESVVSGLDSPAKTSSMEKKLLIKSKELQDSQDKCHKMEQEMTRLHR |
| | RVSEVEAVLSQKEVELKASETQRSLLEQDLATYITECSSLKRSLEQARMEVSQEDDKALQLLHDIREQ |
| | SRKLQEIKEQEYQAQVEEMRLMMNQLEEDLVSARRRSDLYESELRESRLAAEEFKRKATECQHKLLKA |
| | KDQGKPEVGEYAKLEKINAEQQLKIQELQEKLEKAVKASTEATELLQNIRQAKERAERELEKLQNRED |
| | SSEGIRKKLVEAEERRHSLENKVKRLETMERRENRLKDDIOTKSOOIOOMADKILELEEKHREAOVSA |
| | QHLEVHLKQKEQHYEEKIKVLDNQIKKDLADKETLENMMQRHEEEAHEKGKILSEQKAMINAMDSKIR |
| | SLEQRIVELSEANKLAANSSLFTQRNMKAQEEMISELRQQKFYLETQAGKLEAQNRKLEEQLEKISHQ |
| | DHSDKNRLLELETRLREVSLEHEEQKLELKRQLTELQLSLQERESQLTALQAARAALESQLRQAKTEL |
| | EETTAEAEEEIQALTAHRDEIQRKFDALRNSCTVITDLEEQLNQLTEDNAELNNQNFYLSKQLDEASG |
| | ANDEIVQLRSEVDHLRREITEREMQLTSQKQTMEALKTTCTMLEEQVMDLEALNDELLEKERQWEAWR |
| | SVLGDEKSQFECRVRELQRMLDTEKQSRARADQRITESRQVVELAVKEHKAEILALQQALKEQKLKAE |
| | SLSDKLNDLEKKHAMLEMNARSLQQKLETERELKQRLLEEQAKLQQQMDLQKNHIFRLTQGLQEALDR |
| | ADLLKTERSDLEYQLENIQVLYSHEKVKMEGTISQQTKLIDFLQAKMDQPAKKKKVPLQYNELKLALE |
| | KEKARCAELEEALQKTRIELRSAREEAAHRKATDHPHPSTPATARQQIAMSAIVRSPEHQPSAMSLLA |
| | PPSSRRKESSTPEEFSRRLKERMHHNIPHRFNVGLNMRATKCAVCLDTVHFGRQASKCLECQVMCHPK |
| | CSTCLPATCGLPAEYATHFTEAFCRDKMNSPGLQTKEPSSSLHLEGWMKVPRNNKRGQQGWDRKYIVL |
| | EGSKVLIYDNEAREAGQRPVEEFELCLPDGDVSIHGAVGASELANTAKADVPYILKMESHPHTTCWPG |
| | RTLYLLAPSFPDKQRWVTALESVVAGGRVSREKAEADAKLLGNSLLKLEGDDRLDMNCTLPFSDQVVL |
| | VGTEEGLYALNVLKNSLTHVPGIGAVFQIYIIKDLEKLLMIAGEERALCLVDVKKVKQSLAQSHLPAQ |
| | PDISPNIFEAVKGCHLFGAGKIENGLCICAAMPSKVVILRYNENLSKYCIRKEIETSEPCSCIHFTNY |
| | SILIGTNKFYEIDMKQYTLEEFLDKNDHSLAPAVFAASSNSFPVSIVQVNSAGQREEYLLCFHEFGVF |
| | VDSYGRRSRTDDLKWSRLPLAFAYREPYLFVTHFNSLEVIEIQARSSAGTPARAYLDIPNPRYLGPAI |
| | SSGAIYLASSYQDKLRVICCKGNLVKESGTEHHRGPSTSRSSPNKRGPPTYNEHITKRVASSPAPPEG |
| | PSHPREPSTPHRYREGRTELRRDKSPGRPLEREKSPGRMLSTRRERSPGRLFEDSSRGRLPAGAVRTP |
| | LSQVNKVWDQSSV |

| | |
|---|---|
| SEQ ID NO: 3 | 1870 bp |

| | |
|---|---|
| NOV1b, | CACCGGTACCACCATGTTGAAGTTCAAATATGGAGCGCGGAATCCTTTGGATGCTGGTGCTGCTGAA |
| 268667493 | |
| DNA Sequence | CCCATTGCCAGCCGGGCCTCCAGGCTGAATCTGTTCTTCCAGGGGAAACCACCCTTTATGACTCAAC |
| | AGCAGATGTCTCCTCTTTCCCGAGAAGGGATATTAGATGCCCTCTTTGTTCTCTTTGAAGAATGCAG |
| | TCAGCCTGCTCTGATGAAGATTAAGCACGTGAGCAACTTTGTCCGGAAGTATTCCGACACCATGACT |
| | GAGTTACAGGAGCTCCAGCCTTCGGCAAAGGACTTCGAAGTCAGAAGTCTTGTAGGTTGTGGTCACT |
| | TTGCTGAAGTGCAGGTGGTAAGAGAGAAAGCAACCGGGGACATCTATGCTATGAAAGTGATGAAGAA |
| | GAAGGCTTTATTGGCCCAGGAGCAGGTTTCATTTTTTGAGGAAGAGCGGAACATATTATCTCGAAGC |
| | ACAAGCCCGTGGATCCCCCAATTACAGTATGCCTTTCAGGACAAAAATCACCTTTATCTGGTCATGG |
| | AATATCAGCCTGGAGGGGACTTGCTGTCACTTTTGAATAGATATGAGGACCAGTTAGATGAAAACCT |
| | GATACAGTTTTACCTAGCTGAGCTGATTTTGGCTGTTCACAGCGTTCATCTGATGGGATACGTGCAT |
| | CGAGACATCAAGCCTGAGAACATTCTCGTTGACCGCACAGGACACATCAAGCTGGTGGATTTTGGAT |
| | CTGCCGCGAAAATGAATTCAAACAAGATGGTGAATGCCAAACTCCCGATTGGGACCCCAGATTACAT |
| | GGCTCCTGAAGTGCTGACTGTGATGAACGGGGATGGAAAAGGCACCTACGGCCTGGACTGTGACTGG |
| | TGGTCAGTGGGCGTGATTGCCTATGAGATGATTTATGGGAGATCCCCCTTCGCAGAGGGAACCTCTG |
| | CCAGAACCTTCAATAACATTATGAATTTCCAGCGGTTTTTGAAATTTCCAGATGACCCCAAAGTGAG |
| | CAGTGACTTTCTTGATCTGATTCAAAGCTTGTTGTGCGGCCAGAAAGAGAGACTGAAGTTTGAAGGT |
| | CTTTGCTGCCATCCTTTCTTCTCTAAAATTGACTGGAACAACATTCGTAACTCTCCTCCCCCCTTCG |
| | TTCCCACCCTCAAGTCTGACGATGACACCTCCAATTTTGATGAACCAGAGAAGAATTCGTGGGTTTC |
| | ATCCTCTCCGTGCCAGCTGAGCCCCTCAGGCTTCTCGGGTGAAGAACTGCCGTTTGTGGGGTTTTCG |
| | TACAGCAAGGCACTGGGGATTCTTGGTAGATCTGAGTCTGTTGTGTCGGGTCTGGACTCCCCTGCCA |
| | AGACTAGCTCCATGGAAAAGAAACTTCTCATCAAAAGCAAAGAGCTACAAGACTCTCAGGACAAGTG |
| | TCACAAGATGGAGCAGGAAATGACCCGGTTACATCGGAGAGTGTCAGAGGTGGAGGCTGTGCTTAGT |
| | CAGAAGGAGGTGGAGCTGAAGGCCTCTGAGACTCAGAGATCCCTCCTGGAGCAGGACCTTGCTACCT |
| | ACATCACAGAATGCAGTAGCTTAAAGCGAAGTTTGGAGCAAGCACGGATGGAGGTGTCCCAGGAGGA |
| | TGACAAAGCACTGCAGCTTCTCCATGATATCAGAGAGCAGAGCCGGAAGCTCCAAGAAATCAAAGAG |
| | CAGGAGTACCAGGCTCAAGTGGAAGAAATGAGGTTGATGATGAATCAGTTGGAAGAGGATCTTGTCT |
| | CAGCAAGAAGACGGAGTGATCTCTACGAATCTGAGCTGAGAGAGTCTCGGCTTGCTGCTGAAGAATT |
| | CAAGCGGAAAGCGACAGAATGTCAGCATAAACTGTTGAAGGCTAAGGATCAGGTCGACGGC |

| | | |
|---|---|---|
| ORF Start: at 2 | | ORF Stop: end of sequence |
| SEQ ID NO: 4 | 623 aa | MW at 70970.0 kD |

| | |
|---|---|
| NOV1b, | TGTTMLKFKYGARNPLDAGAAEPIASRASRLNLFFQGKPPFMTQQQMSPLSREGILDALFVLFEECS |
| 268667493 | |
| Protein | QPALMKIKHVSNFVRKYSDTIAELQELQPSAKDFEVRSLVGCGHFAEVQVVREKATGDIYAMKVMKK |
| Sequence | |
| | KALLAQEQVSFFEEERNILSRSTSPWIPQLQYAFQDKNHLYLVMEYQPGGDLLSLLNRYEDQLDENL |
| | IQFYLAELILAVHSVHLMGYVHRDIKPENILVDRTGHIKLVDFGSAAKMNSNKMVNAKLPIGTPDYM |
| | APEVLTVMNGDGKGTYGLDCDWWSVGVIAYEMIYGRSPFAEGTSARTFNNIMNFQRFLKFPDDPKVS |
| | SDFLDLIQSLLCGQKERLKFEGLCCHPFFSKIDWNNIRNSPPPFVPTLKSDDDTSNFDEPEKNSWVS |
| | SSPCQLSPSGFSGEELPFVGFSYSKALGILGRSESVVSGLDSPAKTSSMEKKLLIKSKELQDSQDKC |
| | HKMEOEMTRLHRRVSEVEAVLSOKEVELKASETORSLLEODLATYITECSSLKRSLEOARMEVSOED |
| | DKALQLLHDIREQSRKLQEIKEQEYQAQVEEMRLMMNQLEEDLVSARRRSDLYESELRESRLAAEEF |
| | KRKATECQHKLLKAKDQVDG |

| | |
|---|---|
| SEQ ID NO: 5 | 2497 bp |

| | |
|---|---|
| NOV1c, | CACCGGTACCCAGGGGAAGCCTGAAGTGGGAGAATATGCGAAACTGGAGAAGATCAATGCTGAGCAGC |
| 268667539 | |
| DNA Sequence | AGCTCAAAATTCAGGAGCTCCAAGAGAAACTGGAGAAGGCTGTAAAAGCCAGCACGGAGGCCACCGAG |
| | CTGCTGCAGAATATCCGCCAGGCAAAGGAGCGAGCCGAGAGGGAGCTGGAGAAGCTGCAGAACCGAGA |
| | GGATTCTTCTGAAGGCATCAGAAAGAAGCTGGTGGAAGCTGAGGAACGCCGCCATTCTCTGGAGAACA |
| | AGGTAAAGAGACTAGAGACCATGGAGCGTAGAGAAAACAGACTGAAGGATGACATCCAGACAAAATCC |
| | CAACAGATCCAGCAGATGGCTGATAAAATTCTGGAGCTCGAAGAGAAACATCGGGAGGCCCAAGTCTC |
| | AGCCCAGCACCTAGAAGTGCACCTGAAACAGAAAGAGCAGCACTATGAGGAAAAGATTAAAGTGTTGG |
| | ACAATCAGATAAAGAAAGACCTGGCTGACAAGGAGACACTGGAGAACATGATGCAGAGACACGAGGAG |
| | GAGGCCCATGAGAAGGGCAAAATTCTCAGCGAACAGAAGGCGATGATCAATGCTATGGATTCCAAGAT |
| | CAGATCCCTGGAACAGAGGATTGTGGAACTGTCTGAAGCCAATAAACTTGCAGCAAATAGCAGTCTTT |
| | TTACCCAAAGGAACATGAAGGCCCAAGAAGAGATGATTTCTGAACTCAGGCAACAGAAATTTTACCTG |
| | GAGACACAGGCTGGGAAGTTGGAGGCCCAGAACCGAAAACTGGAGGAGCAGCTGGAGAAGATCAGCCA |
| | CCAAGACCACAGTGACAAGAATCGGCTGCTGGAACTGGAGACAAGATTGCGGGAGGTCAGTCTAGAGC |
| | ACGAGGAGCAGAAACTGGAGCTCAAGCGCCAGCTCACAGAGCTACAGCTCTCCCTGCAGGAGCGCGAG |
| | TCACAGTTGACAGCCCTGCAGGCTGCACGGGCGGCCCTGGAGAGCCAGCTTCGCCAGGCGAAGACAGA |
| | GCTGGAAGAGACCACAGCAGAAGCTGAAGAGGAGATCCAGGCACTCACGGCACATAGAGATGAAATCC |
| | AGCGCAAATTTGATGCTCTTCGTAACAGCTGTACTGTAATCACAGACCTGGAGGAGCAGCTAAACCAG |
| | CTGACCGAGGACAACGCTGAACTCAACAACCAAAACTTCTACTTGTCCAAACAACTCGATGAGGCTTC |
| | TGGCGCCAACGACGAGATTGTACAACTGCGAAGTGAAGTGGACCATCTCCGCCGGGAGATCACGGAAC |
| | GAGAGATGCAGCTTACCAGCCAGAAGCAAACGATGGAGGCTCTGAAGACCACGTGCACCATGCTGGAG |
| | GAACAGGTCATGGATTTGGAGGCCCTAAACGATGATCTGCTAGAAAAAGAGCGGCAGTGGGAGGCCTG |
| | GAGGAGCGTCCTGGGTGATGAGAAATCCCAGTTTGAGTGTCGGGTTCGAGAGCTGCAGAGAATGCTGG |
| | ACACCGAGAAACAGAGCAGGGCGAGAGCCGATCAGCGGATCACCGAGTCTCGCCAGGTGGTGGAGCTG |
| | GCAGTGAAGGAGCACAAGGCTGAGATTCTCGCTCTGCAGCAGGCTCTCAAAGAGCAGAAGCTGAAGGC |
| | CGAGAGCCTCTCTGACAAGCTCAATGACCTGGAGAAGAAGCATGCTATGCTTGAAATGAATGCCCGAA |
| | GCTTACAGCAGAAGCTGGAGACTGAACGAGAGCTCAAACAGAGGCTTCTGGAAGAGCAAGCCAAATTA |
| | CAGCAGCAGATGGACCTGCAGAAAAATCACATTTTCCGTCTGACTCAAGGACTGCAAGAAGCTCTAGA |
| | TCGGGCTGATCTACTGAAGACAGAAAGAAGTGACTTGGAGTATCAGCTGGAAAACATTCAGGTTCTCT |
| | ATTCTCATGAAAAGGTGAAAATGGAAGGCACTATTTCTCAACAAACCAAACTCATTGATTTTCTGCAA |
| | GCCAAAATGGACCAACCTGCTAAAAAGAAAAAGGTTCCTCTGCAGTACAATGAGCTGAAGCTGGCCCT |
| | GGAGAAGGAGAAAGCTCGCTGTGCAGAGCTAGAGGAAGCCCTTCAGAAGACCCGCATCGAGCTCCGGT |
| | CCGCCCGGGAGGAAGCTGCCCACCGCAAAGCAACGGACCACCCACACCCATCCACGCCAGCCACCGCG |
| | AGGCAGCAGATCGCCATGTCCGCCATCGTGCGGTCGCCAGAGCACCAGCCCAGTGCCATGAGCCTGCT |
| | GCATGCACCACAATATTCCTCACCGATTCAACGTAGGACTGAACATGCGAGCCACAAAGTGTGCTGTG |
| | TGTCTGGATACCGTGCACTTTGGACGCCAGGCATCCAAATGTCTCGAATGTCAGGTGATGTGTCACCC |
| | CAAGTGCTCCACGTGCTTGCCAGCCACCTGCGGCTTGCCTGTCGACGGC |

| | | |
|---|---|---|
| ORF Start: at 2 | | ORF Stop: end of sequence |
| SEQ ID NO: 6 | 832 aa | MW at 96885.8 kD |

| | |
|---|---|
| NOV1c | TGTQGKPEVGEYAKLEKINAEQQLKIQELQEKLEKAVKASTEATELLQNIRQAKERAERELEKLQNRE |
| 268667539 | |
| Protein | DSSEGIRKKLVEAEERRHSLENKVKRLETMERRENRLKDDIQTKSQQIQQMADKILELEEKHREAQVS |
| Sequence | |
| | AQHLEVHLKQKEQHYEEKIKVLDNQIKKDLADKETLENMMQRHEEEAHEKGKILSEQKAMINAMDSKI |
| | RSLEQRIVELSEANKLAANSSLFTQRNMKAQEEMISELRQQKFYLETQAGKLEAQNRKLEEQLEKISH |
| | QDHSDKNRLLELETRLREVSLEHEEQKLELKRQLTELQLSLQERESQLTALQAARAALESQLRQAKTE |
| | LEETTAEAEEEIQALTAHRDEIQRKFDALRNSCTVITDLEEQLNQLTEDNAELNNQNFYLSKQLDEAS |
| | GANDEIVQLRSEVDHLRREITEREMQLTSQKQTMEALKTTCTMLEEQVMDLEALNDELLEKERQWEAW |
| | RSVLGDEKSQFECRVRELQRMLDTEKQSRARADQRITESRQVVELAVKEHKAEILALQQALKEQKLKA |
| | ESLSDKLNDLEKKHAMLEMNARSLQQKLETERELKQRLLEEQAKLQQQMDLQKNHIFRLTQGLQEALD |
| | RADLLKTERSDLEYQLENIQVLYSHEKVKMEGTISQQTKLIDFLQAKMDQPAKKKKVPLQYNELKLAL |
| | EKEKARCAELEEALQKTRIELRSAREEAAHRKATDHPHPSTPATARQQIAMSAIVRSPEHQPSAMSLL |
| | APPSSRRKESSTPEEFSRRLKERMHHNIPHRFNVGLNMRATKCAVCLDTVHFGRQASKCLECQVMCHP |
| | KCSTCLPATCGLPVDG |

| | |
|---|---|
| SEQ ID NO 7 | 2542 bp |

| | |
|---|---|
| NOV1d, | CACCGGTACCCAGGGGAAGCCTGAAGTGGGAGAATATGCGAAACTGGAGAAGATCAATGCTGAGCAG |
| 268667543 | |
| DNA Sequence | CAGCTCAAAATTCAGGAGCTCCAAGAGAAACTGCAGAAGGCTGTAAAAGCCACCACCGAGGCCACCG |
| | AGCTGCTGCACAATATCCGCCAGGCAAAGGAGCGAGCCGAGAGGGAGCTGGAGAAGCTGCAGAACCG |
| | AGAGGATTCTTCTGAAGGCATCAGAAAGAAGCTGCTGGAAGCTGAGGAACGCCGCCATTCTCTGGAG |
| | AACAAGGTAAAGAGACTAGAGACCATGGAGCCTAGAGAAAACAGACTGAACGATGACATCCAGACAA |
| | AATCCCAACAGATCCAGCAGATGGCTGATAAAATTCTGGACCTCGAAGAGAAACATCGGGAGGCCCA |
| | AGTCTCAGCCCAGCACCTAGAAGTGCACCTGAAACAGAAAGAGCAGCACTATGAGGAAAAGATTAAA |
| | GTGTTGGACAATCAGATAAACAAAGACCTGGCTGACAAGGAGACACTGGAGAACATGATGCAGAGAC |
| | ACGAGGAGGAGGCCCATGAGAAGGGCAAAATTCTCAGCGAACAGAAGGCGATGATCAATGCTATGGA |
| | TTCCAACATCAGATCCCTGGAACAGAGGATTGTGGAACTGTCTGAACCCAATAAACTTCCAGCAAAT |
| | AGCAGTCTTTTTACCCAAAGGAACATGAAGGCCCAAGAAGAGATGATTTCTGAACTCAGGCAACAGA |
| | AATTTTACCTGGAGACACAGGCTGGGAAGTTGGAGGCCCAGAACCGAAAACTGGAGGAGCAGCTGGA |
| | GAAGATCAGCCACCAAGACCACAGTGACAAGAATCGGCTGCTGGAACTGGAGACAAGATTGCGGGAG |
| | GTCAGTCTAGAGCACGAGGAGCAGAAACTGGAGCTCAAGCGCCAGCTCACAGAGCTACAGCTCTCCC |
| | TGCAGGAGCGCGAGTCACAGTTGACAGCCCTGCAGGCTGCACGGGCGGCCCTGGAGAGCCAGCTTCG |
| | CCAGGCGAAGACAGAGCTGGAAGAGACCACAGCAGAAGCTGAAGAGGAGATCCAGGCACTCACGGCA |
| | CATAGAGATGAAATCCAGCGCAAATTTGATGCTCTTCGTAACAGCTGTACTGTAATCACAGACCTGG |
| | AGGAGCAGCTAAACCAGCTGACCGAGGACAACGCTGAACTCAACAACCAAAACTTCTACTTGTCCAA |
| | ACAACTCGATGAGGCTTCTGGCGCCAACGACGAGATTGTACAACTGCGAAGTGAAGTGGACCATCTC |
| | CGCCGGGAGATCACGGAACGAGAGATGCAGCTTACCAGCCAGAAGCAAACGATGGAGGCTCTGAAGA |
| | CCACGTGCACCATGCTGGAGGAACAGGTCATGGATTTGGAGGCCCTAAACGATGAGCTGCTAGAAAA |
| | AGAGCGGCAGTGGGAGGCCTGGAGGAGCGTCCTGGGTGATGAGAAATCCCAGTTTGAGTGTCGGGTT |
| | CGAGAGCTGCAGAGGATGCTGGACACCGAGAAACAGAGCAGGGCGAGAGCCGATCAGCGGATCACCG |
| | AGTCTCGCCAGGTGGTGGAGCTGGCAGTGAAGGAGCACAAGGCTGAGATTCTCGCTCTGCAGCAGGC |
| | TCTCAAAGAGCAGAAGCTGAAGGCCGAGAGCCTCTCTGACAAGCTCAATGACCTGGAGAAGAAGCAT |
| | GCTATGCTTGAAATGAATGCCCGAAGCTTACAGCAGAAGCTGGAGACTGAACGAGAGCTCAAACAGA |
| | GGCTTCTGGAAGAGCAAGCCAAATTACAGCAGCAGATGGACCTGCAGAAAAATCACATTTTCCGTCT |
| | GACTCAAGGACTGCAAGAAGCTCTAGATCGGGCTGATCTACTGAAGACAGAAAGAAGTGACTTGGAG |
| | TATCAGCTGGAAAACATTCAGGTTCTCTATTCTCATGAAAAGGTGAAAATGGAAGGCACTATTTCTC |
| | AACAAACCAAACTCATTGATTTTCTGCAAGCCAAAATGGACCAACCTGCTAAAAAGAAAAAGGGTTT |
| | ATTTAGTCGACGGAAAGAGGACCCTGCTTTACCCACACAGGTTCCTCTGCAGTACAATGAGCTGAAG |
| | CTGGCCCTGGAGAAGGAGAAAGCTCGCTGTGCAGAGCTAGAGGAAGCCCTTCAGAAGACCCGCATCG |
| | AGCTCCGGTCCGCCCGGGAGGAAGCTGCCCACCGCAAAGCAACGGACCACCCACACCCATCCACGCC |
| | AGCCACCGCGAGGCAGCAGATCGCCATGTCTGCCATCGTGCGGTCGCCAGAGCACCAGCCCAGTGCC |
| | ATGAGCCTGCTGGCCCCGCCATCCAGCCGCAGAAAGGAGTCTTCAACTCCAGAGGAATTTAGTCGGC |
| | GTCTTAAGGAACGCATGCACCACAATATTCCTCACCGATTCAACGTAGGACTGAACATGCGAGCCAC |
| | AAAGTGTGCTGTYGTGTCTGGATACCGTGCACTTTGGACGCCAGGATCCAAATGTCTCGAATGTCAG |
| | GTGATGTGTCACCCCAAGTGCTCCACGTGCTTGCCAGCCACCTGCGGCTTGCCTGTCGACGGC |

| | | |
|---|---|---|
| ORF Start: at 2 | | ORF Stop: end of sequence |
| SEQ ID NO: 8 | 847 aa | MW at 98582.7 kD |

| | |
|---|---|
| NOV1d | TGTQGKPEVGEYAKLEKINAEQQLKIQELQEKLEKAVKASTEATELLQNIRQAKERAERELEKLQNR |
| 268667543 | |
| Protein | EDSSEGIRKKLVEAEERRHSLENKVKRLETMERRENRLKDDIQTKSQQIQQMADKILELEEKHREAQ |
| Sequence | |
| | VSAQHLEVHLKQKEQHYEEKIKVLDNQIKKDLADKETLENMMQRHEEEAHEKGKILSEQKAMINAMD |
| | SKIRSLEQRIVELSEANKLAANSSLFTQRNMKAQEEMISELRQQKFYLETQAGKLEAQNRKLEEQLE |
| | KISHQDHSDKNRLLELETRLREVELEHEEQKLELKRQLTELQLSLQERESQLTALQAARAALESQLR |
| | QAKTELEETTAEAEEEIQALTAHRDEIQRKFDALRNSCTVITDLEEQLNQLTEDNAELNNQNFYLSK |
| | QLDEASGANDEIVQLRSEVDHLRREITEREMQLTSQKQTMEALKTTCTMLEEQVMDLEALNDELLEK |
| | ERQWEAWRSVLGDEKSQFECRVRELQRMLDTEKQSRARADQRITESRQVVELAVKEHKAEILALQQA |
| | LKEQKLKAESLSDKLNDLEKKHAMLEMNARSLQQKLETERELKQRLLEEQAKLQQQMDLQKNHIFRL |
| | TQGLQEALDRADLLKTERSDLEYQLENIQVLYSHEKVKMEGTISQQTKLIDFLQAKMDQPAKKKKGL |
| | FSRRKEDPALPTQVPLQYNELKLALEKEKARCAELEEALQKTRIELRSAREEAAHRKATDHPHPSTP |
| | ATARQQIAMSAIVRSPEHQPSAMSLLAPPSSRRKESSTPEEFSRRLKERMHHNIPHRFNVGLNMRAT |
| | KCAVCLDTVHFGRQASKCLECQVMCHPKCSTCLPATCGLPVDG |

| | |
|---|---|
| SEQ ID NO: 9 | 1870 bp |

| | |
|---|---|
| NOV1e, | CACCGGTACCTGCGGCTTGCCTGCTGAATATGCCACACACTTCACCGAGGCCTTCTGCCGTGACAAAA |
| 268667555 | |
| DNA Sequence | TGAACTCCCCAGGTCTCCAGACCAAGGAGCCCAGCAGCAGCTTGCACCTGGAAGGGTGGATGAAGGTG |
| | CCCAGGAATAACAAACGAGGACAGCAAGGCTGGGACAGGAAGTACATTGTCCTGGAGGGATCAAAAGT |
| | CCTCATTTATGACAATGAAGCCAGAGAAGCTGGACAGAGGCCGGTGGAAGAATTTGAGCTGTGCCTTC |
| | CCGACGGGGATGTATCTATTCATGGTGCCGTTGGTGCTTCCGAACTCGCAAATACAGCCAAAGCAGAT |
| | GTCCCATACATACTGAAGATGGAATCTCACCCGCACACCACCTGCTGGCCCGGGAGAACCCTCTACTT |
| | GCTAGCTCCCAGCTTCCCTGACAAACAGCGCTGGGTCACCGCCTTAGAATCAGTTGTCGCAGGTGGGA |
| | GAGTTTCTAGGGAAAAAGCAGAAGCTGATGCTAAACTGCTTGGAAACTCCCTGCTGAAACTGGAAGGT |
| | GATGACCGTCTAGACATGAACTGCACGCTGCCCTTCAGTGACCAGGTGGTGTTGGTGGGTACCGAGGA |
| | AGGGCTCTACGCCCTGAATGTCTTGAAAAACTCCCTAACCCATGTCCCAGGAATTGGAGCAGTCTTCC |
| | AAATTTATATTATCAAGGACCTGGAGAAGCTACTCATGATAGCAGGAGAAGAGCGGGCACTGTGTCTT |
| | GTGGACGTGAAGAAAGTGAAACAGTCCCTGGCCCAGTCCCACCTGCCTGCCCAGCCCGACATCTCACC |
| | CAACATTTTTGAAGCTGTCAAGGGCTGCCACTTGTTTGGGGCAGGCAAGATTGAGAACGGGCTCTGCA |
| | TCTGTGCAGCCATGCCCAGCAAAGTCGTCATTCTCCGCTACAACGAAAACCTCAGCAAATACTGCATC |
| | CGGAAAGAGATAGAGACCTCAGAGCCCTGCAGCTGTATCCACTTCACCAATTACAGTATCCTCATTGG |
| | AACCAATAAATTCTACGAAATCGACATGAAGCAGTACACGCTCGAGGAATTCCTGGATAAGAATGACC |
| | ATTCCTTGGCACCTGCTGTGTTTGCCGCCTCTTCCAACAGCTTCCCTGTCTCAATCGTGCAGGTGAAC |
| | AGCGCAGGGCAGCGAGAGGAGTACTTGCTGTGTTTCCACGAATTTGGAGTGTTCGTGGATTCTTACGG |
| | AAGACGTAGCCGCACAGACGATCTCAAGTGGAGTCGCTTACCTTTGGCCTTTGCCTACAGAGAACCCT |
| | ATCTGTTTGTGACCCACTTCAACTCACTCGAAGTAATTGAGATCCAGGCACGCTCCTCAGCAGGGACC |
| | CCTGCCCGAGCGTACCTGGACATCCCGAACCCGCGCTACCTGGGCCCTGCCATTTCCTCAGGAGCGAT |
| | TTACTTGGCGTCCTCATACCAGGATAAATTAAGGGTCATTTGCTGCAAGGGAAACCTCGTGAAGGAGT |
| | CCGGCACTGAACACCACCGGGGCCCGTCCACCTCCCGCAGCAGCCCCAACAAGCGAGGCCCACCCACG |
| | TACAACGAGCACATCACCAAGCGCGTGGCCTCCAGCCCAGCGCCGCCCGCAAGGCCCAGCCACCCGCG |
| | AGAGCCAAGCACACCCCACCGCTACCGCGAGGGGCGGACCGAGCTGCGCAGGGACAAGTCTCCTGGCC |
| | GCCCCCTGGAGCGAGAGAAGTCCCCCGGCCGGATGCTCAGCACGCGGAGAGAGCGGTCCCCCGGGAGG |
| | CTGTTTGAAGACAGCAGCAGGGGCCGGCTGCCTGCGGGAGCCGTGAGGACCCCGCTGTCCCAGGTGAA |
| | CAAGGTGTGGGACCAGTCTTCAGTAGTCGACGGC |

| | | |
|---|---|---|
| ORF Start: at 2 | | ORF Stop: end of sequence |
| SEQ ID NO: 10 | 623 aa | MW at 69278.9 kD |

| | |
|---|---|
| NOV1e, | TGTCGLPAEYATHFTEAFCRDKMNSPGLQTKEPSSSLHLEGWMKVPRNNKRGQQGWDRKYIVLEGSKV |
| 268667555 | |
| Protein | LIYDNEAREAGQRPVEEFELCLPDGDVSIHGAVGASELANTAKADVPYILKMESHPHTTCWPGRTLYL |
| Sequence | |
| | LAPSFPDKQRWVTALESVVVAGGRVSREKAEDAKLLGNSLLKLEGDDRLDMNCTLPFSDQVVLVGTEE |
| | GLYALNVLKNSLTHVPGIGAVFQIYIIKDLEKLLMIAGEERALCLVDVKKVKQSLAQSHLPAQPDISP |
| | NIFEAVKGCHLFGAGKIENGLCICAAMPSKVVILRYNENLSKYCIRKEIETSEPCSCIHFTNYSILIG |
| | TNKFYEIDMKQYTLEEFLDKNDHSLAPAVFAASSNSFPVSIVQVNSAGQREEYLLCFHEFGVFVDSYG |
| | RRSRTDDLKWSRLPLAFAYREPYLFVTHFNSLEVIEIQARSSAGTPARAYLDIPNPRYLGPAISSGAI |
| | YLASSYQDKLRVICCKGNLVKESGTEHHRGPSTSRSSPNKRGPPTYNEHITKRVASSPAPPEGPSHPR |
| | EPSTPHRYREGRTELRRDKSPGRPLEREKSPGRMLSTRRERSPGRLFEDSSRGRLPAGAVRTPLSQVN |
| | KVWDQSSVVDG |

| | |
|---|---|
| SEQ ID NO: 11 | 1915 bp |

| | |
|---|---|
| NOV1f, | CACCGGTACCTGCGGCTTGCCTGCTGAATATGCCACACACTTCACCGAGGCCTTCTGCCGTGATAAAA |
| 268667574 | |
| DNA Sequence | TGAACTCCCCAGGTCTCCAGACCAAGGAGCCCAGCAGCAGCTTGCACCTGGAAGGGTGGATGAAGGTG |
| | CCCAGGAATAACAAACGAGGACAGCAAGGCTGGGACAGGAAGTACATTGTCCTGGAGGGATCAAAAGT |
| | CCTCATTTATGACAATGAAGCCAGAGAAGCTGGACAGAGGCCGGTGGAAGAATTTGAGCTGTGCCTTC |
| | CCGACGGGGATGTATCTATTCATGGTGCCGTTGGTGCTTCCGAACTCGCAAATACAGCCAAAGCAGAT |
| | GTCCCATACATACTGAAGATGGAATCTCACCCGCACACCACCTGCTGGCCCGGGAGAACCCTCTACTT |
| | GCTAGCTCCCAGCTTCCCTGACAAACAGCGCTGGGTCACCGCCTTAGAATCAGTTGTCGCAGGTGGGA |
| | GAGTTTCTAGGGAAAAAGCAGAAGCTGATGCTGCCCGCGACTGTGTTTCTTACGAGCTTCTGCCTGCC |
| | TGGGTTCAGAAACTGCTTGGAAACTCCCTGCTGAAACTGGAAGGTGATGACCGTCTAGACATGAACTG |
| | CACACTGCCCTTCAGTGACCAGGTGGTGTTGGTGGGCACCGAGGAAGGGCTCTACGCCCTGAATGTCT |
| | TGAAAAACTCCCTAACCCATGTCCCAGGAATTGGAGCAGTCTTCCAAATTTATATTATCAAGGACCTG |
| | GAGAAGCTACTCATGATAGCAGGAGAAGAGCGGGCACTGTGTCTTGTGGACGTGAAGAAAGTGAAACA |
| | GTCCCTGGCCCAGTCCCACCTGCCTGCCCAGCCCGACATCTCACCCAACATTTTTGAAGCTGTCAAGG |
| | GCTGCCACTTGTTTGGGGCAGGCAAGATTGAGAACGGGCTCTGCATCTGTGCAGCCATGCCCAGCAAA |
| | GTCGTCATTCTCCGCTACAACGAAAACCTCAGCAAATACTGCATCCGGAAAGAGATAGAGACCTCAGA |
| | GCCCTGCAGCTGTATCCACTTCACCAATTACAGTATCCTCATTGGAACCAATAAATTCTACGAAATCG |
| | ACATGAAGCAGTACACGCTCGAGGAATTCCTGGATAAGAATGACCATTCCTTGGCACCTGCTGTGTTT |
| | GCCGCCTCTTCCAACAGCTTCCCTGTCTCAATCGTGCAGGTGAACAGCGCAGGGCAGCGAGAGGAGTA |
| | GTTGCTGTGTTTCCACGAATTTGGAGTGTTCGTGGATTCTTACGGAAGACGTAGCCGCACAGACGATC |
| | TCAAGTGGAGTCGCTTACCTTTGGCCTTTGCCTACAGAGAACCCTATCTGTTTGTGACCCACTTCAAC |
| | TCACTCGAAGTAATTGAGATCCAGGCACGCTCCTCAGCAGGGACCCCTGCCCGAGCGTACCTGGACAT |
| | CCCGAACCCGCGCTACCTGGGCCCTGCCATTTCCTCAGGAGCGATTTACTTGGCGTCCTCATACCAGG |
| | ATAAATTAAGGGTCATTTGCTGCAAGGGAAACCTCGTGAAGGAGTCCGGCACTGAACACCACCGGGGC |
| | CCGTCCACCTCCCGCAGCAGCCCCAACAAGCGAGGCCCACCCACGTACAACGAGCACATCACCAAGCG |
| | CGTGGCCTCCAGCCCAGCGCCGCCCGAAGGCCCCAGCCACCCGCGAGAGCCAAGCACACCCCACCGCT |
| | ACCGCGAGGGGCGGACCGAGCTGCGAGGGACAAGTCTCCTGGCCGCCCCCCTGGAGCGAGAGAAGTCC |
| | CCCGGCCGGATGCTCAGCACGCGGAGAGAGCGGTCCCCCGGGAGGCTGTTTGAAGACAGCAGCAGGGG |
| | CCGGCTGCCTGCGGGAGCCGTGAGGACCCCGCTGTCCCAGGTGAACAAGGTGTGGGACCAGTCTTCAG |
| | TAGTCGACGGC |

| | | |
|---|---|---|
| ORF Start: at 2 | | ORF Stop: end of sequence |
| SEQ ID NO: 12 | 638 aa | MW at 71010.8 kD |

| | |
|---|---|
| NOV1f, | TGTCGLPAEYATHFTEAFCRDKMNSPGLQTKEPSSSLHLEGWMKVPRNNKRGQQGWDRKYIVLEGSKV |
| 268667574 | |
| Protein | LIYDNEAREAGQRPVEEFELCLPDGDVSIHGAVGASELANTAKADVPYILKMESHPHTTCWPGRTLYL |
| Sequence | |
| | LAPSFPDKQRWVTALESVVAGGRVSREKAEADAARDCVSYELLPAWVQKLLGNSLLKLEGDDRLDMNC |
| | TLPFSDQVVLVGTEEGLYALNVLKNSLTHVPGIGAVFQIYIIKDLEKLLMIAGEERALCLVDVKKVKQ |
| | SLAQSHLPAQPDISPNIFEAVKGCHLFGAGKIENGLCICAAMPSKVVILRYNENLSKYCIRKEIETSE |
| | PCSCIHFTNYSILIGTNKFYEIDMKQYTLEEFLDKNDHSLAPAVFAASSNSFPVSIVQVNSAGQREEY |
| | LLCFHEFGVFVDSYGRRSRTDDLKWSRLPLAFAYREPYLFVTHFNSLEVIEIQARSSAGTPARAYLDI |
| | PNPRYLGPAISSGAIYLASSYQDKLRVICCKGNLVKESGTEHHRGPSTSRSSPNKRGPPTYNEHITKR |
| | VASSPAPPEGPSHPREPSTPHRYREGRTELRRDKSPGRPLEREKSPGRMLSTRRERSPGRLFEDSSRG |
| | RLPAGAVRTPLSQVNKVWDQSSVVDG |

| | |
|---|---|
| SEQ ID NO: 13 | 6201 bp |

| | |
|---|---|
| NOV1g, | ATGTTGAAGTTCAAATATGGAGCGCGGAATCCTTTGGATGCTGGTGCTGCTGAACCCATTGCCAGCC |
| CG106764-02 | |
| DNA Sequence | GGGCCTCCAGGCTGAATCTGTTCTTCCAGGGGAAACCACCCTTTATGACTCAACAGCAGATGTCTCC |
| | TCTTTCCCGAGAAGGGATATTAGATGCCCTCTTTGTTCTCTTTGAAGAATGCAGTCAGCCTGCTCTG |
| | ATGAAGATTAAGCACGTGAGCAACTTTGTCCGGAAGTGTTCCGACACCATAGCTGAGTTACAGGAGC |
| | TCCAGCCTTCGGCAAAGGACTTCGAAGTCAGAAGTCTTGTAGGTTGTGGTCACTTTGCTGAAGTGCA |
| | GGTGGTAAGAGAGAAAGCAACCGGGGACATCTATGCTATGAAAGTGATGAAGAAGAAGGCTTTATTG |
| | GCCCAGGAGCAGGTTTCATTTTTTGAGGAAGAGCGGAACATATTATCTCGAAGCACAAGCCCGTGGA |
| | TCCCCCAATTACAGTATGCCTTTCAGGACAAAAATCACCTTTATCTGGTGATGGAATATCAGCCTGG |
| | AGGGGACTTGCTGTCACTTTTGAATAGATATGAGGACCAGTTAGATGAAAACCTGATACAGTTTTAC |
| | CTAGCTGAGCTGATTTTGGCTGTTCACAGCGTTCATCTGATGGGATACGTGCATCGGGACATCAAGC |
| | CTGAGAACATTCTCGTTGACCGCACAGGACACATCAAGCTGGTGGATTTTGGATCTGCCGCGAAAAT |
| | GAATTCAAACAAGGTGAATGCCAAACTCCCGATTGGGACCCCAGATTACATGGCTCCTGAAGTGCTG |
| | ACTGTGATGAACGGGGATGGAAAAGGCACCTACGGCCTGGACTGTGACTGGTGGTCAGTGGGCGTGA |
| | TTGCCTATGAGATGATTTATGGGAGATCCCCCTTCGCAGAGGGAACCTCTGCCAGAACCTTCAATAA |
| | CATTATGAATTTCCAGCGGTTTTTGAAATTTCCAGATGACCCCAAAGTGAGCAGTGACTTTCTTGAT |
| | CTGATTCAAAGCTTGTTGTGCGGCCAGAAAGAGAGACTGAAGTTTGAAGGTCTTTGCTGCCATCCTT |
| | TCTTCTCTAAAATTGACTGGAACAACATTCGTAACGCTCCTCCCCCCTTCGTTCCCACCCTCAAGTC |
| | TGACGATGACACCTCCAATTTTGATGAACCAGAGAAGAATTCGTGGGTTTCATCCTCTCCGTGCCAG |
| | CTGAGCCCCTCAGGCTTCTCGGGTGAAGAACTGCCGTTTGTGGGGTTTTCGTACAGCAAGGCACTGG |
| | GGATTCTTGGTAGATCTGAGTCTGTTGTGTCGGGTCTGGACTCCCCTGCCAAGACTAGCTCCATGGA |
| | AAAGAAACTTCTCATCAAAAGCAAAGAGCTACAAGACTCTCAGGACAAGTGTCACAAGATGGAGCAG |
| | GAAATGACCCGGTTACATCGGAGAGTGTCAGAGGTGGAGGCTGTGCTTAGTCAGAAGGAGGTGGAGC |
| | TGAAGGCCTCTGAGACTCAGAGATCCCTCCTGGAGCAGGACCTTGCTACCTACATCACAGAATGCAG |
| | TAGCTTAAAGCGAAGTTTGGAGCAAGCACGGATGGAGGTGTCCCAGGAGGATGACAAAGCACTGCAG |
| | CTTCTCCATGATATCAGAGAGCAGAGCCGGAAGCTCCAAGAAATCAAAGAGCAGGAGTACCAGGCTC |
| | AAGTGGAAGAAATGAGGTTGATGATGAATCAGTTGGAAGAGGATCTTGTCTCAGCAAGAAGACGGAG |
| | TGATCTCTACGAATCTGAGCTGAGAGAGTCTCGGCTTGCTGCTGAAGAATTCAAGCGGAAAGCGACA |
| | GAATGTCAGCATAAACTGTTGAAGGCTAAGGATCAGGGGAAGCCTGAAGTGGGAGAATATGCGAAAC |
| | TGGAGAAGATCAATGCTGAGCAGCAGCTCAAAATTCAGGAGCTCCAAGAGAAACTGGAGAAGGCTGT |
| | AAAAGCCAGCACGGAGGCCACCGAGCTGCTGCAGAATATCCGCCAGGCAAAGGAGCGAGCCGAGAGG |
| | GAGCTGGAGAAGCTGCAGAACCGAGAGGATTCTTCTGAAGGCATCAGAAAGAAGCTGGTGGAAGCTG |
| | AGGAACGCCGCCATTCTCTGGAGAACAAGGTAAAGAGACTAGAGACCATGGAGCGTAGAGAAAACAG |
| | ACTGAAGGATGACATCCAGACAAAATCCCAACAGATCCAGCAGATGGCTGATAAAATTCTGGAGCTC |
| | GAAGAGAAACATCGGGAGGCCCAAGTCTCAGCCCAGCACCTAGAAGTGCACCTGAAACAGAAAGAGC |
| | AGCACTATGAGGAAAAGATTAAAGTATTGGACAATCAGATAAAGAAAGACCTGGCTGACAAGGAGAC |
| | ACTGGAGAACATGATGCAGAGACACGAGGAGGAGGCCCATGAGAAGGGCAAAATTCTCAGCGAACAG |
| | AAGGCGATGATCAATGCTATGGATTCCAAGATCAGATCCCTGGAACAGAGGATTGTGGAACTGTCTG |
| | AAGCCAATAAACTTGCAGCAAATAGCAGTCTTTTTACCCAAAGGAACATGAAGGCCCAAGAAGAGAT |
| | GATTTCTGAACTCAGGCAACAGAAATTTTACCTGGAGACACAGGCTGGGAAGTTGGAGGCCCAGAAC |
| | CGAAAACTGGAGGAGCAGCTGGAGAAGATCAGCCACCAAGACCACAGTGACAAGAATCGGCTGCTGG |
| | AACTGGAGACAAGATTGCGGGAGGTGAGTCTAGAGCACGAGGAGCAGAAACTGGAGCTCAAGCGCCA |
| | GCTCACAGAGCTACAGCTCTCCCTGCAGGAGCGCGAGTCACAGTTGACAGCCCTGCAGGCTGCACGG |
| | GCGGCCCTGGAGAGCCAGCTTCGCCAGGCGAAGACAGAGCTGGAAGAGACCACAGCAGAAGCTGAAG |
| | AGGAGATCCAGGCACTCACGGCACATAGAGATGAAATCCAGCGCAAATTTGATGCTCTTCGTAACAG |
| | CTGTACTGTGATCACAGACCTGGAGGAGCAGCTAAACCAGCTGACCGAGGACAACGCTGAACTCAAC |
| | AACCAAAACTTCTACTTGTCCAAACAACTCGATGAGGCTTCTGGCGCCAACGACGAGATTGTACAAC |
| | TGCGAAGTGAAGTGGACCATCTCCGCCGGGAGATCACGGAACGAGAGATGCAGCTTACCAGCCAGAA |
| | GCAAACGATGGAGGCTCTGAAGACCACGTGCACCATGCTGGAGGAACAGGTCATGGATTTGGAGGCC |
| | CTAAACGATGAGCTGCTAGAAAAAGAGCGGCAGTGGGAGGCCTGGAGGAGCGTCCTGGGTGATGAGA |
| | AATCCCAGTTTGAGTGTCGGGTTCGAGAGCTGCAGAGGATGCTGGACACCGAGAAACAGAGCAGGGC |
| | GAGAGCCGATCAGCGGATCACCGAGTCTCGCCAGGTGGTGGAGCTGGCAGTGAAGGAGCACAAGGCT |
| | GAGATTCTCGCTCTGCAGCAGGCTCTCAAAGAGCAGAAGCTGAAGGCCGAGAGCCTCTCTGACAAGC |
| | TCAATGACCTGGAGAAGAAGCATGCTATGCTTGAAATGAATGCCCGAAGCTTACAGCAGAAGCTGGA |
| | GACTGAACGAGAGCTCAAACAGAGGCTTCTGGAAGAGCAAGCCAAATTACAGCAGCAGATGGACCTG |
| | CAGAAAAATCACATTTTCCGTCTGACTCAAGGACTGCAAGAAGCTCTAGATCGGGCTGATCTACTGA |
| | AGACAGAAAGAAGTGACTTGGAGTATCAGCTGGAAAACATTCAGGTGCTCTATTCTCATGAAAAGGT |
| | GAAAATGGAAGGCACTATTTCTCAACAAACCAAACTCATTGATTTTCTGCAAGCCAAAATGGACCAA |
| | CCTGCTAAAAAGAAAAAGGTGCCTCTGCAGTACAATGAGCTGAAGCTGGCCCTGGAGAAGGAGAAAG |
| | CTCGCTGTGCAGAGCTAGAGGAAGCCCTTCAGAAGACCCGCATCGAGCTCCGGTCCGCCCGGGAGGA |
| | AGCTGCCCACCGCAAAGCAACGGACCACCCACACCCATCCACGCCAGCCACCGCGAGGCAGCAGATC |
| | GCCATGTCTGCCATCGTGCGGTCGCCAGAGCACCAGCCCAGTGCCATGAGCCTGCTGGCCCCGCCAT |
| | CCAGCCGCAGAAAGGAGTCTTCAACTCCAGAGGAATTTAGTCGGCGTCTTAAGGAACGCATGCACCA |
| | CAATATTCCTCACCGATTCAACGTAGGACTGAACATGCGAGCCACAAAGTGTGCTGTGTGTCTGGAT |
| | ACCGTGCACTTTGGACGCCAGGCATCCAAATGTCTAGAATGTCAGGTGATGTGTCACCCCAAGTGCT |
| | CCACGTGCTTGCCAGCCACCTGCGGCTTGCCTGCTGAATATGCCACACACTTCACCGAGGCCTTCTG |
| | CCGTGACAAAATGAACTCCCCAGGTCTCCAGACCAAGGAGCCCAGCAGCAGCTTGCACCTGGAAGGG |
| | TGGATGAAGGTGCCCAGGAATAACAAACGAGGACAGCAAGGCTGGGACAGGAAGTACATTGTCCTGG |
| | AGGGATCAAAAGTCCTCATTTATGACAATGAAGCCAGAGAAGCTGGACAGAGGCCGGTGGAAGAATT |
| | TGAGCTGTGCCTTCCCGACGGGGATGTATCTATTCATGGTGCCGTTGGTGCTTCCGAACTCGCAAAT |
| | ACAGCCAAAGCAGATGTCCCATACATACTGAAGATGGAATCTCACCCGCACACCACCTGCTGGCCCG |
| | GGAGAACCCTCTACTTGCTAGCTCCCAGCTTCCCTGACAAACAGCGCTGGGTCACCGCCTTAGAATC |
| | AGTTGTCGCAGGTGGGAGAGTTTCTAGGGAAAAAGCAGAAGCTGATGCTAAACTGCTTGGAAACTCC |
| | CTGCTGAAACTGGAAGGTGATGACCGTCTAGACATGAACTGCACGCTGCCCTTCAGTGACCAGGTAG |
| | TGTTGGTGGGCACCGAGGAAGGGGCTCTACGCCTGGATGTCTTGAAAAACTCCCTAACCCATGTCCC |
| | AGGAATTGGAGCAGTCTTCCAAATTTATATTATCAAGGACCTGGAGAAGCTACTCATGATAGCAGGT |
| | GAAGAGCGGGCACTGTGTCTTGTGGACGTGAAGAAAGTGAAACAGTCCCTGGCCCAGTCCCACCTGC |
| | CTGCCCAGCCCGACATCTCACCCAACATTTTTGAAGCTGTCAAGGGCTGCCACTTGTTTGGGGCAGG |
| | CAAGATTGAGAACGGGCTCTGCATCTGTGCAGCCATGCCCAGCAAAGTCGTCATTCTCCGCTACAAC |
| | GAAAACCTCAGCAAATACTGCATCCGGAAAGAGATAGAGACCTCAGAGCCCTGCAGCTGTATCCACT |
| | TCACCAATTACAGTATCCTCATTGGAACCAATAAATTCTACGAAATCGACATGAAGCAGTACACGCT |
| | CGAGGAATTCCTGGATAAGAATGACCATTCCTTGGCACCTGCTGTGTTTGCCGCCTCTTCCAACAGC |
| | TTCCCTGTCTCAATCGTGCAGGTGAACAGCGCAGGGCAGCGAGAGGAGTACTTGCTGTGTTTCCACG |
| | AATTTGGAGTGTTCGTGGATTCTTACGGAAGACGTAGCCGCACAGACGATCTCAAGTGGAGTCGCTT |
| | ACCTTTGGCCTTTGCCTACAGAGAACCCTATCTGTTTGTGACCCACTTCAACTCACTCGAAGTAATT |
| | GAGATCCAGGCACGCTCCTCAGCAGGGACCCCTGCCCGAGCGTACCTGGACATCCCGAACCCGCGCT |
| | ACCTGGGCCCTGCCATTTCCTCAGGAGCGATTTACTTGGCGTCCTCATACCAGGATAAATTAAGGGT |
| | CATTTGCTGCAAGGGAAACCTCGTGAAGGAGTCCGGCACTGAACACCACCGGGGCCCGTCCACCTCC |
| | CGCAGCAGCCCCAACAAGCGAGGCCCACCCACGTACAACGAGCACATCACCAAGCGCGTGGCCTCCA |
| | GCCCAGCGCCGCCCGAAGGCCCCAGCCACCCGCGAGAGCCAAGCACACCCCACCGCTACCGCGAGGG |
| | GCGGACCGAGCTGCGCAGGGACAAGTCTCCTGGCCGCCCCCTGGAGCGAGAGAAGTCCCCCGGCCGG |
| | ATGCTCAGCACGCGGAGAGAGCGGTCCCCCGGGAGGCTGTTTGAAGACAGCAGCAGGGGCCGGCTGC |
| | CTGCGGGAGCCGTGAGGACCCCGCTGTCCCAGGTGAACAAGGTGAGGCAGCATTCCGAGGCCTGTGT |
| | GTCTGTTGCGGAGGCCAGGAGTGACTTGGGGAACTGA |

| | | |
|---|---|---|
| ORF Start: ATG at 1 | | ORF Stop: TGA at 6199 |
| SEQ ID NO: 14 | 2066 aa | MW at 236008.5 kD |

| | |
|---|---|
| NOV1g, | MLKFKYGARNPLDAGAAEPIASRASRLNLFFQGKPPFMTQQQMSPLSREGILDALFVLFEECSQPAL |
| CG106764-02 | |
| Protein | MKIKHVSNFVRKCSDTIAELQELQPSAKDFEVRSLVGCGHFAEVQVVREKATGDIYAMKVMKKKALL |
| Sequence | |
| | AQEQVSFFEEERNILSRSTSPWIPQLQYAFQDKNHLYLVMEYQPGGDLLSLLNRYEDQLDENLIQFY |
| | LAELILAVHSVHLMGYVHRDIKPENILVDRTGHIKLVDFGSAAKMNSNKVNAKLPIGTPDYMAPEVL |
| | TVMNGDGKGTYGLDCDWWSVGVIAYEMIYGRSPFAEGTSARTFNNIMNFQRFLKFPDDPKVSSDFLD |
| | LIQSLLCGQKERLKFEGLCCHPFFSKIDWNNIRNAPPPFVPTLKSDDDTSNFDEPEKNSWVSSSPCQ |
| | LSPSGFSGEELPFVGFSYSKALGILGRSESVVSGLDSPAKTSSMEKKLLIKSKELODSODKCHKMEO |
| | EMTRLHRRVSEVEAVLSQKEVELKASETQRSLLEQDLATYITECSSLKRSLEQARMEVSQEDDKALQ |
| | LLHDIREQSRKLQEIKEQEYQAQVEEMRLMMNQLEEDLVSARRRSDLYESELRESRLAAEEFKRKAT |
| | ECQHKLLKAKDQGKPEVGEYAKLEKINAEQQLKIQELQEKLEKAVKASTEATELLQNIRQAKERAER |
| | ELEKLQNREDSSEGIRKKLVEAEERRHSLENKVKRLETMERRENRLKDDIQTKSQQIQQMADKILEL |
| | EEKHREAQVSAQHLEVHLKQKEQHYEEKIKVLDNQIKKDLADKETLENMMQRHEEEAHEKGKILSEQ |
| | KAMINAMDSKIRSLEQRIVELSEANKLAANSSLFTQRNMKAQEEMISELRQQKFYLETQAGKLEAQN |
| | RKLEEQLEKISHQDHSDKNRLLELETRLREVSLEHEEQKLELKRQLTELQLSLQERESQLTALQAAR |
| | AALESQLRQAKTELEETTAEAEEEIQALTAHRDEIQRKFDALRNSCTVITDLEEQLNQLTEDNAELN |
| | NQNFYLSKQLDEASGANDEIVQLRSEVDHLRREITEREMQLTSQKQTMEALKTTCTMLEEQVMDLEA |
| | LNDELLEKERQWEAWRSVLGDEKSQFECRVRELQRMLDTEKQSRARADQRITESRQVVELAVKEHKA |
| | EILALQQALKEQKLKAESLSDKLNDLEKKHAMLEMNARSLQQKLETERELKQRLLEEQAKLQQQMDL |
| | QKNHIFRLTQGLQEALDRADLLKTERSDLEYQLENIQVLYSHEKVKMEGTISQQTKLIDFLQAKMDQ |
| | PAKKKKVPLQYNELKLALEKEKARCAELEEALQKTRIELRSAREEAAHRKATDHPHPSTPATARQQI |
| | AMSAIVRSPEHQPSAMSLLAPPSSRRKESSTPEEFSRRLKERMHHNIPHRFNVGLNMRATKCAVCLD |
| | TVHFGRQASKCLECQVMCHPKCSTCLPATCGLPAEYATHFTEAFCRDKMNSPGLQTKEPSSSLHLEG |
| | WMKVPRNNKRGQQGWDRKYIVLEGSKVLIYDNEAREAGQRPVEEFELCLPDGDVSIHGAVGASELAN |
| | TAKADVPYILKMESHPHTTCWPGRTLYLLAPSFPDKQRWVTALESVVAGGRVSREKAEADAKLLGNS |
| | LLKLEGDDRLDMNCTLPFSDQVVLVGTEEGLYALNVLKNSLTHVPGIGAVFQIYIIKDLEKLLMIAG |
| | EERALCLVDVKKVKQSLAQSHLPAQPDISPNIFEAVKGCHLFGAGKIENGLCICAAMPSKVVILRYN |
| | ENLSKYCIRKEIETSEPCSCIHFTNYSILIGTNKFYEIDMKQYTLEEFLDKNDHSLAPAVFAASSNS |
| | FPVSIVQVNSAGQREEYLLCFHEFGVFVDSYGRRSRTDDLKWSRLPLAFAYREPYLFVTHFNSLEVI |
| | EIQARSSAGTPARAYLDIPNPRYLGPAISSGAIYLASSYQDKLRVICCKGNLVKESGTEHHRGPSTS |
| | RSSPNKRGPPTYNEHITKRVASSPAPPEGPSHPREPSTPHRYREGRTELRRDKSPGRPLEREKSPGR |
| | MLSTRRERSPGRLFEDSSRGRLPAGAVRTPLSQVNKVRQHSEACVSVAEARSDLGN |

Sequence comparison of the above protein sequences yields the following sequence relationships shown in Table 1B.

TABLE 1B
Comparison of NOV1a against NOV1b through NOV1g.

| | NOV1a Residues/ | Identities/Similarities |
|---|---|---|
| Protein Sequence | Match Residues | for the Matched Region |
| NOV1b | 1 . . . 615 | 601/616 (97%) |
| | 5 . . . 620 | 602/616 (97%) |
| NOV1c | 615 . . . 1442 | 690/828 (83%) |
| | 4 . . . 831 | 691/828 (83%) |
| NOV1d | 615 . . . 1442 | 690/843 (81%) |
| | 4 . . . 846 | 691/843 (81%) |
| NOV1e | 1436 . . . 2053 | 618/618 (100%) |
| | 3 . . . 620 | 618/618 (100%) |
| NOV1f | 1436 . . . 2053 | 618/633 (97%) |
| | 3 . . . 635 | 618/633 (97%) |
| NOV1g | 1 . . . 2051 | 1900/2051 (92%) |
| | 1 . . . 2051 | 1900/2051 (92%) |

Further analysis of the NOV1a protein yielded the following properties shown in Table 1C.

TABLE 1C
Protein Sequence Properties NOV1a

| | |
|---|---|
| PSort analysis: | 0.9800 probability located in nucleus; |
| | 0.3000 probability located in microbody (peroxisome); |
| | 0.1000 probability located in mitochondrial matrix |
| | space; 0.1000 probability located in lysosome (lumen) |
| SignalP analysis: | No Known Signal Sequence Predicted |

A search of the NOVIa protein against the Geneseq database, a proprietary database that contains sequences published in patents and patent publication, yielded several homologous proteins shown in Table 1D.

TABLE 1D
Geneseq Results for NOV1a

| | | NOV1a | | |
|---|---|---|---|---|
| | | Residues/ | Identities/ | |
| Geneseq | Protein/Organism/Length | Match | Similarities for the | Expect |
| Identifier | [Patent #, Date] | Residues | Matched Region | Value |
| AAU03501 | Human protein kinase #1 - | 1 . . . 2051 | 2044/2053 (99%) | 0.0 |
| | Homo sapiens, 2053 aa. | 1 . . . 2053 | 2046/2053 (99%) | |
| | [WO200138503-A2, | | | |
| | 31-MAY-2001 | | | |
| AAB43359 | Human ORFX ORF3123 | 768 . . . 2053 | 1286/1286 (100%) | 0.0 |
| | polypeptide sequence SEQ | 1 . . . 1286 | 1286/1286 (100%) | |
| | ID NO: 6246 - Homo | | | |
| | sapiens, 1286 aa. | | | |
| | [WO200058473-A2, | | | |
| | 05-OCT-2000] | | | |
| ABB11117 | Human RHO/RAC effector | 968 . . . 1947 | 976/980 (99%) | 0.0 |
| | homologue, SEQ ID | 1 . . . 980 | 976/980 (99%) | |
| | NO: 1487 - Homo sapiens, | | | |
| | 999 aa. | | | |
| | [WO200157188-A2, | | | |
| | 09-AUG-2001] | | | |
| AAU31443 | Novel human secreted | 1114 . . . 1982 | 867/869 (99%) | 0.0 |
| | protein #1934 - Homo | 1 . . . 869 | 867/869 (99%) | |
| | sapiens, 910 aa. | | | |
| | [WO200179449-A2, | | | |
| | 25-OCT-2001] | | | |
| AAE16261 | Human kinase PKIN-7 | 1 . . . 467 | 463/468 (98%) | 0.0 |
| | protein - Homo sapiens, | 1 . . . 468 | 465/468 (98%) | |
| | 497 aa. | | | |
| | [WO200196547-A2, | | | |
| | 20-DEC-2001] | | | |

In a BLAST search of public sequence datbases, the NOV1a protein was found to have homology to the proteins shown in the BLASTP data in Table 1E.

TABLE 1E
Public BLASTP Results for NOV1a

| | | NOV1a | | |
|---|---|---|---|---|
| Protein | | Residues/ | Identities/ | |
| Accession | | Match | Similarities for the | Expect |
| Number | Protein/Organism/Length | Residues | Matched Portion | Value |
| O88938 | Rho/rac-interacting citron | 1 . . . 2053 | 1974/2055 (96%) | 0.0 |
| | kinase - Mus musculus | 1 . . . 2055 | 2014/2055 (97%) | |
| | (Mouse), 2055 aa. | | | |
| O88528 | Citron-K kinase - Mus | 373 . . . 2053 | 1599/1683 (95%) | 0.0 |
| | musculus (Mouse), 1641 aa | 1 . . . 1641 | 1616/1683 (96%) | |
| | (fragment). | | | |
| P49025 | Citron protein | 467 . . . 2053 | 1563/1589 (98%) | 0.0 |
| | (Rho-interacting, | 9 . . . 1597 | 1578/1589 (98%) | |
| | serine/threonine kinase 21) - | | | |
| | Mus musculus (Mouse), | | | |
| | 1597 aa. | | | |
| Q9QX19 | Postsynaptic density protein - | 467 . . . 2053 | 1556/1619 (96%) | 0.0 |
| | Rattus norvegicus (Rat), | 1 . . . 1618 | 1573/1619 (97%) | |
| | 1618 aa. | | | |
| O14578 | Citron protein | 768 . . . 2053 | 1286/1286 (100%) | 0.0 |
| | (Rho-interacting, | 1 . . . 1286 | 1286/1286 (100%) | |
| | serine/threonine kinase 21) - | | | |
| | Homo sapiens (Human), | | | |
| | 1286 aa (fragment). | | | |

PFam analysis predicts that the NOV1a protein contains the domains shown in the Table 1F.

TABLE 1F
Domain Analysis of NOV1a

| | | Identities/ | |
|---|---|---|---|
| | NOV1a | Similarities for | |
| Pfam Domain | Match Region | the Matched Region | Expect Value |
| pkinase | 97 . . . 359 | 89/302 (29%) | 2.7e−62 |
| | | 196/302 (65%) | |
| pkinase_C | 360 . . . 389 | 15/32 (47%) | 0.00023 |
| | | 24/32 (75%) | |
| DAG_PE-bind | 1389 . . . 1437 | 14/51 (27%) | 6.1e−05 |
| | | 34/51 (67%) | |
| PH | 1470 . . . 1589 | 20/121 (17%) | 1.8e−11 |
| | | 87/121 (72%) | |
| CNH | 1618 . . . 1915 | 107/378 (28%) | 1.5e−110 |
| | | 289/378 (76%) | |

### Example 2

The NOV2 clone was analyzed, and the nucleotide and encoded polypeptide sequences are shown in Table 2A.

TABLE 2A
NOV2 Sequence Analysis

| | |
|---|---|
| SEQ ID NO: 15 | 1238 bp |

| | |
|---|---|
| NOV2a, | ATGGATGGATGGAGAAGGATGCCTCGCTGGGGACTGCTGCTGCTGCTCTGGGGCTCCTGTACCTTTGG |
| CG117662-01 | |
| DNA Sequence | TCTCCCGACAGACACCACCACCTTTAAACGGATCTTCCTCAAGAGAATGCCCTCAATCCGAGAAAGCC |
| | TGAAGGAACGAGGTGTGGACATGGCCAGGCTTGGTCCCGAGTGGAGCCAACCCATGAAGAGGCTGACA |
| | CTTGGCAACACCACCTCCTCCGTGATCCTCACCAACTACATGGACACCCAGTACTATGGCGAGATTGG |
| | CATCGGCACCCCACCCCAGACCTTCAAAGTCGTCTTTGACACTGGTTCGTCCAATGTTTGGGTGCCCT |
| | CCTCCAAGTGCAGCCGTCTCTACACTGCCTGTGTGTATCACAAGCTCTTCGATGCTTCGGATTCCTCC |
| | AGCTACAAGCACAATGGAACAGAACTCACCCTCCGCTATTCAACAGGGACAGTCAGTGGCTTTCTCAG |
| | CCAGGACATCATCACCGTGGGTGGAATCACGGTGACACAGATGTTTGGAGAGGTCACGGAGATGCCCG |
| | CCTTACCCTTCATGCTGGCCGAGTTTGATGGGGTTGTGGGCATGGGCTTCATTGAACAGGCCATTGGC |
| | AGGGTCACCCCTATCTTCGACAACATCATCTCCCAAGGGGTGCTAAAAGAGGACGTCTTCTCTTTCTA |
| | CTACAACAGAGATTCCGAGAATTCCCAATCGCTGGGAGGACAGATTGTGCTGGGAGGCAGCGACCCCC |
| | AGCATTACGAAGGGAATTTCCACTATATCAACCTCATCAAGACTGGTGTCTGGCAGATTCAAATGAAG |
| | GGGGTGTCTGTGGGGTCATCCACCTTGCTCTGTGAAGACGGCTGCCTGGCATTGGTAGACACCGGTGC |
| | ATCCTACATCTCAGGTTCTACCAGCTCCATAGAGAAGCTCATGGAGGCCTTGGGAGCCAAGAAGAGGC |
| | TGTTTGATTATGTCGTGAAGTGTAACGAGGGCCCTACACTCCCCGACATCTCTTTCCACCTGGGAGGC |
| | AAAGAATACACGCTCACCAGCGCGGACTATGTATTTCAGGAATCCTACAGTAGTAAAAAGCTGTGCAC |
| | ACTGGCCATCCACGCCATGGATATCCCGCCACCCACTGGACCCACCTGGGCCCTGGGGGCCACCTTCA |
| | TCCGAAAGTTCTACACAGAGTTTGATCGGCGTAACAACCGCATTGGCTTCGCCTCGGCCCGCTGAGGC |
| | CCTCTGCCACCCAG |

| | | |
|---|---|---|
| ORF Start: ATG at 1 | | ORF Stop: TGA at 1219 |
| SEQ ID NO: 16 | 406 aa | MW at 45030.9 kD |

| | |
|---|---|
| NOV2a, | MDGWRRMPRWGLLLLLWGSCTFGLPTDTTTFKRIFLKRMPSIRESLKERGVDMARLGPEWSQPMKRLT |
| CG117662-01 | |
| Protein | LGNTTSSVILTNYMDTQYYGEIGIGTPPQTFKVVFDTGSSNVWVPSSKCSRLYTACVYHKLFDASDSS |
| Sequence | |
| | SYKHNGTELTLRYSTGTVSGFLSQDIITVGGITVTQMFGEVTEMPALPFMLAEFDGVVGMGFIEQAIG |
| | RVTPIFDNIISQGVLKEDVFSFYYNRDSENSQSLGGQIVLGGSDPQHYEGNFHYINLIKTGVWQIQMK |
| | GVSVGSSTLLCEDGCLALVDTGASYISGSTSSIEKLMEALGAKKRLFDYVVKCNEGPTLPDISFHLGG |
| | KEYTLTSADYVFQESYSSKKLCTLAIHAMDIPPPTGPTWALGATFIRKFYTEFDRRNNRIGFASAR |

| | |
|---|---|
| SEQ ID NO: 17 | 911 bp |

| | |
|---|---|
| NOV2b, | ATGGATGGATGGAGAAGGATGCCTCGCTGGGGACTGCTGCTGCTGCTCTGGGGCTCCTGTACCTTTG |
| CG117662-02 | |
| DNA Sequence | GTCTCCCGACAGACACCACCACCTTTAAACGGATCTTCCTCAAGAGAATGCCCTCAATCCGAGAAAG |
| | CCTGAAGGAACGAGGTGTGGACATGGCCAGGCTTGGTCCCGAGTGGAGCCAACCCATGAAGAGGCTG |
| | ACACTTGGCAACACCACCTCCTCCGTGATCCTCACCAACTACATGGACACCCAGTACTATGGCGAGA |
| | TTGGCATCGGCACCCCACCCCAGACCTTCAAAGTCGTCTTTGACACTGGTTCGTCCAATGTTTGGGT |
| | GCCCTCCTCCAAGTGCAGCCGTCTCTACACTGCCTGTGTGTATCACAAGCTCTTCGATGCTTCGGAT |
| | TCCTCCAGCTACAAGCACAATGGAACAGAACTCACCCTCCGCTATTCAACAGGGACAGTCAGTGGCT |
| | TTCTCAGCCAGGACATCATCACCGTGTCTGTGGGGTCATCCACCTTACTCTGTGAAGACGGCTGCCT |
| | GGCATTGGTAGACACCGGTGCATCCTACATCTCAGGTTCTACCAGCTCCATAGAGAAGCTCATGGAG |
| | GCCTTGGGAGCCAAGAAGAGGCTGTTTGATTATGTCGTGAAGTGTAACGAGGGCCCTACACTCCCCG |
| | ACATCTCTTTCCACCTGGGAGGCAAAGAATACACGCTCACCAGCGCGGACTATGTATTTCAGGAATC |
| | CTACAGTAGTAAAAAGCTGTGCACACTGGCCATCCACGCCATGGATATCCCGCCACCCACTGGACCC |
| | ACCTGGGCCCTGGGGGCCACCTTCATCCGAAAGTTCTACACAGAGTTTGATCGGCGTAACAACCGCA |
| | TTGGCTTCGCCTCGGCCCGCTGAGGCCCTCTGCCACCCAG |

| | | |
|---|---|---|
| ORF Start: ATG at 1 | | ORF Stop: TGA at 892 |
| SEQ ID NO: 18 | 297 aa | MW at 33025.3 kD |

| | |
|---|---|
| NOV2b, | MDGWRRMPRWGLLLLLWGSCTFGLPTDTTTFKRIFLKRMPSIRESLKERGVDMARLGPEWSQPMKRL |
| CG117662-02 | |
| Protein | TLGNTTSSVILTNYMDTGYYGEIGIGTPPQTFKVVFDTGSSNVWVPSSKCSRLYTACVYHKLFDASD |
| Sequence | |
| | SSSYKHNGTELTLRYSTGTVSGFLSQDIITVSVGSSTLLCEDGCLALVDTGASYISGSTSSIEKLME |
| | ALGAKKRLFDYVVKCNEGPTLPDISFHLGGKEYTLTSADYVFQESYSSKKLCTLAIHAMDIPPPTGP |
| | TWALGATFIRKFYTEFDRRNNRIGFASAR |

Sequence comparison of the above protein sequences yields the following sequence relationships shown in Table 2B.

TABLE 2B
Comparison of NOV2a against NOV2b.

| Protein Sequence | NOV2a Residues/ | Identities/Similarities |
|---|---|---|
| | Match Residues | for the Matched Region |
| NOV2b | 1 . . . 165 | 165/165 (100%) |
| | 1 . . . 165 | 165/165 (100%) |

Further analysis of the NOV2a protein yielded the following properties shown in Table 2C.

TABLE 2C
Protein Sequence Properties NOV2a

| | |
|---|---|
| PSort analysis: | 0.3700 probability located in outside; |
| | 0.2541 probability located in microbody (peroxisome); |
| | 0.1900 probability located in lysosome (lumen); |
| | 0.1000 probability located in endoplasmic |
| | reticulum (membrane) |
| SignalP analysis: | Cleavage site between residues 24 and 25 |

A search of the NOV2a protein against the Geneseq database, a proprietary database that contains sequences published in patents and patent publication, yielded several homologous proteins shown in Table 2D.

TABLE 2D
Geneseq Results for NOV2a

| | | NOV2a | Identities/ | |
|---|---|---|---|---|
| | | Residues/ | Similarities | |
| Geneseq | Protien/Organism/Length | Match | for the Matched | Expect |
| Identifier | [Patent #, Date] | Residues | Region | value |
| AAW23244 | Human renin - Homo sapiens, | 1 . . . 406 | 404/406 (99%) | 0.0 |
| | 406 aa. [WO9728684-A1, | 1 . . . 406 | 404/406 (99%) | |
| | 14-AUG-1997] | | | |
| AAP50135 | Sequence of pre-pro-renin - | 1 . . . 406 | 404/406 (99%) | 0.0 |
| | Homo sapiens, 406 aa. | 1 . . . 406 | 404/406 (99%) | |
| | [EP135347-A, | | | |
| | 27-MAR-1985] | | | |
| ABB11781 | Human renin homologue, | 1 . . . 406 | 391/408 (95%) | 0.0 |
| | SEQ ID NO: 2151 - Homo | 31 . . . 438 | 393/408 (95%) | |
| | sapiens, 438 aa. | | | |
| | [WO200157188-A2, | | | |
| | 09-AUG-2001] | | | |
| AAU72879 | Human aspartyl protease | 24 . . . 405 | 169/400 (42%) | 1e−90 |
| | partial protein sequence #4 - | 14 . . . 409 | 246/400 (61%) | |
| | Homo sapiens, 412 aa. | | | |
| | [WO200183782-A2, | | | |
| | 08-NOV-2001] | | | |
| AAY93685 | Amino acid sequence of | 24 . . . 405 | 169/400 (42%) | 1e−90 |
| | novel polypeptide PRO292 - | 14 . . . 409 | 246/400 (61%) | |
| | Homo sapiens, 412 aa. | | | |
| | [WO200037640-A2, | | | |
| | 29-JUN-2000] | | | |

In a BLAST search of public sequence datbases, the NOV2a protein was found to have homology to the proteins shown in the BLASTP data in Table 2E.

TABLE 2E
Public BLASTP Results for NOV2a

| | | NOV2a | Identities/ | |
|---|---|---|---|---|
| Protein | | Residues/ | Similarities for | |
| Accession | | Match | the Matched | Expect |
| Number | Protein/Organism/Length | Residues | Portion | Value |
| P00797 | Renin precursor, renal (EC | 1 . . . 406 | 405/406 (99%) | 0.0 |
| | 3.4.23.15) | 1 . . . 406 | 405/406 (99%) | |
| | (Angiotensinogenase) - Homo | | | |
| | sapiens (Human), 406 aa. | | | |
| Q9TSZ1 | Preprorenin precursor (EC | 1 . . . 406 | 381/406 (93%) | 0.0 |
| | 3.4.23.15) - Callithrix jacchus | 1 . . . 400 | 389/406 (94%) | |
| | (Common marmoset), 400 aa. | | | |
| P52115 | renin precursor, renal (EC | 7 . . . 406 | 292/401 (72%) | e−175 |
| | 3.4.23.15) | 1 . . . 400 | 338/401 (83%) | |
| | (Angiotensinogenase) - Ovis | | | |
| | aries (Sheep), 400 aa. | | | |
| Q15296 | Kidney mRNA fragment for | 108 . . . 406 | 297/300 (99%) | e−172 |
| | renin (Aa 105-401) - Homo | 1 . . . 300 | 298/300 (99%) | |
| | sapiens (Human), 300 aa | | | |
| | (fragment). | | | |
| P06281 | Renin precursor, renal (BC | 5 . . . 406 | 281/403 (69%) | e−167 |
| | 3.4.23.15) | 4 . . . 402 | 331/403 (81%) | |
| | (Angiotensinogenase) - Mus | | | |
| | musculus (Mouse), 402 aa. | | | |

PFam analysis predicts that the NOV2a protein contains the domains shown in the Table 2F.

TABLE 2F
Domain Analysis of NOV2a

| | | Identities/ | |
|---|---|---|---|
| | NOV2a | Similarities for | |
| Pfam Domain | Match Region | the Matched Region | Expect Value |
| asp | 31 . . . 405 | 174/428(41%) | 4.le−197 |
| | | 339/428 (79%) | |

### Example 3

The NOV3 clone was analyzed, and the nucleotide and encoded polypeptide sequences are shown in Table 3A.

TABLE 3A
NOV3 Sequence Analysis

| | |
|---|---|
| SEQ ID NO:19 | 2827 bp |

| | |
|---|---|
| NOV3a, | TGGCGATGCTACTGTTTAATTGCAGGAGGTGGGGGTGTGTGTACCATGTACCAGGGCTATTAGAAGCA |
| CG118051-01 | |
| DNA Sequence | AGAAGGAAGGAGGGAGGGCAGAGCGCCCTGCTGAGCAACAAAGGACTCCTGCAGCCTTCTCTGTCTGT |
| | CTCTTGGCACAGGCACATGGGGAGGCCTCCCGCAGGTGGGGGGCCACCAGTCCAGGGGTGGGAGCACT |
| | ACAGGGCACGAGTTGGTTTGGGAGCTGCCAGTCTCCTGGGAGGATCGCAGTCAGCAGAGCAGGGCTGA |
| | GGCCTGGGGGTAGGAGCAGAGCCTGCGCATCTGGAGGCAGCATGTCCAAGAAAGGGAGTGGAGGTGCA |
| | GCGAAGGACCCAGGGGCAGAGCCCACGCTGGGGATGGACCCCTTCGAGGACACACTGCGGCGGCTGCG |
| | TGAGGCCTTCAACTGAGGGCGCACGCGGCCGGCCGAGTTCCGGGCTGCGCAGCTCCAGGGCCTGGGCC |
| | ACTTCCTTCAAGAAAACAAGCAGCTTCTGCGCGACGTGCTGGCCCAGGACCTGCATAAGCCAGCTTTC |
| | GAGGCAGACATATCTGAGCTCATCCTTTGCCAGAACGAGGTTGACTACGCTCTCAAGAACCTTCAGGC |
| | CTGGATGAAGGATGAACCACGGTCCACGAACCTGTTCATGAAGCTGGACTCGGTCTTCATCTGGAAGG |
| | AACCCTTTGGCCTGGTCCTCATCATCGCACCCTGGAACTACCCATTGAACCTGACCCTGGTGCTCCTG |
| | GTGGGCACCCTCCCCGCAGGGAATTGCGTGGTGCTGAAGCCGTCAGAAATCAGCCAGGGCACAGAGAA |
| | GGTCCTGGCTGAGGTGCTGCCCCAGTACCTGGACCAGAGCTGCTTTGCCGTGGTGCTGGGCGGACCCC |
| | AGGAGACAGGGCAGCTGCTAGAGCACAAGTTGGACTACATCTTCTTCACAGGGAGCCCTCGTGTGGGC |
| | AAGATTGTCATGACTGCTGCCACCAAGCACCTGACGCCTGTCACCCTGGAGCTGGGGGGCAAGAACCC |
| | CTGCTACGTGGACGACAACTGCGACCCCCAGACCGTGGCCAACCGCGTGGCCTGGTTCTGCTACTTCA |
| | ATGCCGGCCAGACCTGCGTGGCCCCTGACTACGTCCTGTGCAGCCCCGAGATGCAGGAGAGGCTGCTG |
| | CCCGCCCTGCAGAGCACCATCACCCGTTTCTATGGCGACGACCCCCAGAGCTCCCCAAACCTGGGCCG |
| | CATCATCAACCAGAAACAGTTCCAGCGGCTGCGGGCATTGCTGGGCTGCGGCCGCGTGGCCATTGGGG |
| | GCCAGAGCAACGAGAGCGATCGCTACATCGCCCCCACGGTGCTGGTGGACGTGCAGGAGACGGAGCCT |
| | GTGATGCAGGAGGAGATCTTCGGGCCCATCCTGCCCATCGTGAACGTGCAGAGCGTGGACGAGGCCAT |
| | CAAGTTCATCAACCGGCAGGAGAAGCCCCTGGCCCTGTACGCCTTCTCCAACAGCAGACAGGTTGTGA |
| | ACCAGATGCTGGAGCGGACCAGCAGCGGCAGCTTTGGAGGCAATGAGGGCTTCACCTACATATCTCTG |
| | CTGTCCGTGCCATTCGGGGGAGTCGGCCACAGTGGGATGGGCCGGTACCACGGCAAGTTCACCTTCGA |
| | CACCTTCTCCCACCACCGCACCTGCCTGCTCGCCCCCTCCGGCCTGGAGAAATTAAAGGAGATCCGCT |
| | ACCCACCCTATACCGACTGGAACCAGCAGCTGTTACGCTGGGGCATGGGCTCCCAGAGCTGCACCCTC |
| | CTGTGAGCGTCCCACCCGCCTCCAACGGGTCACACAGAGAAACCTGAGTCTAGCCATGAGGGGCTTAT |
| | GCTCCCAACTCACATTGTTCCTCCAGACCGCAGGCTCCCCCAGCCTCAGGTTGCTGGAGCTGTCACAT |
| | GACTGCATCCTGCCTGCCAGGGCTGCAAAGCAAGGTCTTGCTTCTATCTGGGGGACGCTGCTCGAGAG |
| | AGGCCGAGAGGCCGCAGAACATGCCAGGTGTCCTCACTCACCCCACCCTCCCCAATTCCAGCCCTTTG |
| | CCCTCTCGGTCAGGGTTGGCCAGGCCCAGTCACAGGGGCAGTGTCACCCTGGAAAATACAGTGCCCTG |
| | CCTTCTTAGGGGCATCAGCCCTGAACGGTTGAGAGCGTGGAGCCCTCCAGGCCTTTGCTCTCCCCTCT |
| | AGGCACACGCGCACTTCCACCTCTGCCCCATCCCAACTGCACCAGCACTGCCTCCCCCAGGGATCCTC |
| | TCACATCCCACACTGGTCTCTGCACCACCCCTCTGGTTCACACCGCACCCTGCACTCACCCACAGCAG |
| | CTCCATCCACTGGGAAAACTGGGGTTTGCATCACTCCACTGCACAGTGTTAGTGGGACCTGGGGGCAA |
| | GTCCCTTGACTTCTCTGAGCCTCAGTTTCCTTATGTGAAAGTTGCTGGAACCAAAATGGAGTCACTTA |
| | TGCCAAACTCTAATAAAATGGAGTCGGGGGGGCACATAGAAGCCCTCACACACACATGCCCGTAACAG |
| | GATTTATCACCAAGACACGCCTGCATGTAAGACCAGACACAGGGCGTATGGAAAAGCACGTCCTCAAA |
| | GACTGTAGTATTCCAGATGAGCTGCAGATGCTTACCTACCACGGCCGTCTCCACCAGAAAACCATCGC |
| | CAACTCCTGCGATCAGCTTGTGACTTACAAACCTTGTTTAAAAGCTGCTTACATGGACTTCTGTCCTT |
| | TAAAACGTTCCCCTTGGCTGTGGCCCTCTGTGTATGCCTGGGATCCTTCCAAGCACTCATAGCCCAGA |
| | TAGGAATCCTCTGCTCCTCCCAAATAAATTCATCTGTTC |

| | | |
|---|---|---|
| ORF Start: ATG at 617 | | ORF Stop: TGA at 1772 |
| SEQ ID NO: 20 | 385 aa | MW at 42794.8 kD |

| | |
|---|---|
| NOV3a, | MKDEPRSTNLFMKLDSVFIWKEPFGLVLIIAPWNYPLNTLTVLLVGTLPAGNCVVLKPSEISQGTEKV |
| CG118051-01 | |
| Protein | LAEVLPQYLDQSCFAVVLGGPQETGQLLEHKLDYIFFTGSPRVGKIVMTAATKHLTPVTLELGGKNPC |
| Sequence | |
| | YVDDNCDPQTVANRVAWFCYFNAGQTCVAPDYVLCSPEMQERLLPALQSTITRFYGDDPQSSPNLGRI |
| | INQKQFQRLRALLGCGRVAIGGQSNESDRYIAPTVLVDVQETEPVMQEEIFGPILPIVNVQSVDEAIK |
| | FINRQEKPLALYAFSNSRQVVNQMLERTSSGSFGGNEGFTYISLLSVPFGGVGHSGMGRYHGKFTFDT |
| | FSHHRTCLLAPSGLEKLKEIRYPPYTDWNQQLLRWGMGSQSCTLL |

| | |
|---|---|
| SEQ ID NO:21 | 1586 bp |

| | |
|---|---|
| NOV3b, | CACGAGTTGGTTTGGGAGCTGCCAGTCTCCTGGGAGGATCGCAGTCAGCAGAGCAGGGCTGAGGCCT |
| CG118051-02 | |
| DNA Sequence | GGGGGTAGGAGCAGAGCCTGCGCATCTGGAGGCAGCATGTCCAAGAAAGGGAGTGGAGGTGCAGCGA |
| | AGGACCCAGGGGCAGAGCCCACGCTGGGGATGGACCCCTTCGAGGACACACTGCGGCGGCTGCGTGA |
| | GGCCTTCAACTGAGGGCGCACGCGGCCGGCCGAGTTCCGGGCTGCGCAGCTCCAGGGCCTGGGCCAC |
| | TTCCTTCAAGAAAACAAGCAGCTTCTGCGCGACGTGCTGGCCCAGGACCTGCATAAGCCAGCTTTCG |
| | AGGCAGACATATCTGAGCTCATCCTTTGCCAGAACGAGGTTGACTACGCTCTCAAGAACCTTCAGGC |
| | CTGGATGAAGGATGAACCACGGTCCACGAACCTGTTCATGAAGCTGGACTCGGTCTTCATCTGGAAG |
| | GAACCCTTTGGCCTGGTCCTCATCATCGCACCCTGGAACTACCCATTGAACCTGACCCTGGTGCTCC |
| | TGGTGGGCACCCTCCCCGCAGGGAATTGCGTGGTGCTGAAGCCGTCAGAAATCAGCCAGGGCACAGA |
| | GAAGGTCCTGGCTGAGGTGCTGCCCCAGTACCTGGACCAGAGCTGCTTTGCCGTGGTGCTGGGCGGA |
| | CCCCAGGAGACAGGGCAGCTGCTAGAGCACAAGTTGGACTACATCTTCTTCACAGGGAGCCCTCGTG |
| | TGGGCAAGATTGTCATGACTGCTGCCACCAAGCACCTGACGCCTGTCACCCTGGAGCTGGGGGGCAA |
| | GAACCCCTGCTACGTGGACGACAACTGCGACCCCCAGACCGTGGCCAACCGCGTGGCCTGGTTCTGC |
| | TACTTCAATGCCGGCCAGACCTGCGTGGCCCCTGACTACGTCCTGTGCAGCCCCGAGATGCAGGAGA |
| | GGCTGCTGCCCGCCCTGCAGAGCACCATCACCCGTTTCTATGGCGACGACCCCCAGAGCTCCCCAAA |
| | CCTGGGCCGCATCATCAACCAGAAACAGTTCCAGCGGCTGCGGGCATTGCTGGGCTGCGGCCGCGTG |
| | GCCATTGGGGGCCAGAGCAACGAGAGCGATCGCTACATCGCCCCCACGGTGCTGGTGGACGTGCAGG |
| | AGACGGAGCCTGTGATGCAGGAGGAGATCTTCGGGCCCATCCTGCCCATCGTGAACGTGCAGAGCGT |
| | GGACGAGGCCATCAAGTTCATCAACCGGCAGGAGAAGCCCCTGGCCCTGCACAGTGGGATGGGCCGG |
| | TACCACGGCAAGTTCACCTTCGACACCTTCTCCCACCACCGCACCTGCCTGCTCGCCCCCTCCGGCC |
| | TGGAGAAATTAAAGGAGATCCGCTACCCACCCTATACCGACTGGAACCAGCAGCTGTTACGCTGGGG |
| | CATGGGCTCCCAGAGCTGCACCCTCCTGTGAGCGTCCCACCCGCCTCCAACGGGTCACACAGAGAAA |
| | CCTGAGTCTAGCCATGAGGGGCTTATGCTCCCAACTCACATTGTTCCTCCAGACCGCAGGCTCCCCC |
| | AGCCTCAGGTTGCTGGAGCTGTCACATGACTGCATCCTGCCTGCC |

| | | |
|---|---|---|
| ORF Start: ATG at 407 | | ORF Stop: TGA at 1436 |
| SEQ ID NO: 22 | 343 aa | MW at 38350.9 kD |

| | |
|---|---|
| NOV3b, | MKDEPRSTNLFMKLDSVFIWKEPFGLVLIIAPWNYPLNLTLVLLVGTLPAGNCVVLKPSEISQGTEK |
| CG118051-02 | |
| Protein | VLAEVLPQYLDQSCFAVVLGGPQETGQLLEHKLDYIFFTGSPRVGKIVMTAATKHLTPVTLELGGKN |
| Sequence | |
| | PCYVDDNCDPQTVANRVAWFCYFNAGQTCVAPDYVLCSPEMQERLLPALQSTITRFYGDDPQSSPNL |
| | GRIINQKQFQRLRALLGCGRVAIGGQSNESDRYIAPTVLVDVQETEPVMQEEIFGPILPIVNVQSVD |
| | EAIKFINRQEKPLALHSGMGRYHGKFTFDTFSHHRTCLLAPSGLEKLKEIRYPPYTDWNQQLLRWGM |
| | GSQSCTLL |

| | |
|---|---|
| SEQ ID NO:23 | 1791 bp |

| | |
|---|---|
| NOV3c | TTAAGGAGAATCTTAAAGTGAGGGCTGAGGGACTCTCCTGATCCAGAGCTGAGGACTCTCCTGATCCA |
| CG118051-03 | |
| DNA Sequence | GAGCTGAGGGCTCTCCTGATGGACCCCTTCGAGGACACGCTGCGGCGGCTGCGTGAGGCCTTCAACTG |
| | AGGGCGCACGCGGCCGGCCGAGTTCCGGGCTGCGCAGCTCCAGGGCCTGGGCCACTTCCTTCAAGAAA |
| | ACAAGCAGCTTCTGCGCGACGTGCTGGCCCAGGACCTGCATAAGCCAGCTTTCGAGGCAGACATATCT |
| | GAGCTCATCCTTTGCCAGAACGAGGTTGACTACGCTCTCAAGAACCTTCAGGCCTGGATGAAGGATGA |
| | ACCACGGTCCACGAACCTGTTCATGAAGCTGGACTCGGTCTTCATCTGGAAGGAACCCTTTGGCCTGG |
| | TCCTCATCATCGCACCCTGGAACTACCCACTGAACCTGACCCTGGTGCTCCTGGTGGGCGCCCTCGCC |
| | GCAGGGAATTGCGTGGTGCTGAAGCCGTCAGAAATCAGCCAGGGCACAGAGAAGGTCCTGGCTGAGGT |
| | GCTGCCCCAGTACCTGGACCAGAGCTGCTTTGCCGTGGTGCTGGGCGGACCCCAGGAGACAGGGCAGC |
| | TGCTAGAGCACAAGTTGGACTACATCTTCTTCACAGGGAGCCCTCGTGTGGGCAAGATTGTCATGACT |
| | GCTGCCACCAAGCACCTGACGCCTGTCACCCTGGAGCTGGGGGGCAAGAACCCCTGCTACGTGGACGA |
| | CAACTGCGACCCCCAGACCGTGGCCAACCGCGTGGCCTGGTTCTGCTACTTCAATGCCGGCCAGACCT |
| | GCGTGGCCCCTGACTACGTCCTGTGCAGCCCCGAGATGCAGGAGAGGCTGCTGCCCGCCCTGCAGAGC |
| | ACCATCACCCGTTTCTATGGCGACGACCCCCAGAGCTCCCCAAACCTGGGCCGCATCATCAACCAGAA |
| | ACAGTTCCAGCGGCTGCGGGCATTGCTGGGCTGCGGCCGCGTGGCCATTGGGGGCCAGAGCAACGAGA |
| | GCGATCGCTACATCGCCCCCACGGTGCTGGTGGACGTGCAGGAGACGGAGCCTGTGATGCAGGAGGAG |
| | ATCTTCGGGCCCATCCTGCCCATCGTGAACGTGCAGAGCGTGGACGAGGCCATCAAGTTCATCAACCG |
| | GCAGGAGAAGCCCCTGGCCCTGTACGCCTTCTCCAACAGCAGCCAGGTTGTGAACCAGATGCTGGAGC |
| | GGACCAGCAGCGGCAGCTTTGGAGGCAATGAGGGCTTCACCTACATATCTCTGCTGTCCGTGCCATTC |
| | GGGGGAGTCGGCCACAGTGGGATGGGCCGGTACCACGGCAAGTTCACCTTCGACACCTTCTCCCACCA |
| | CCGCACCTGCCCGCTCGCCCCCTCCGGCCTGGAGAAATTAAAGGAGATCCGCTACCCACCCTATACCG |
| | ACTGGAACCAGCAGCTGTTACGCTGGGGCATGGGCTCCCAGAGCTGCACCCTCCTGTGAGCGTCCCAC |
| | CCGCCTCCAACGGGTCACACAGAGAAACCTGAGTCTAGCCATGAGGGGCTTATGCTCCCAACTCACAT |
| | TGTTCCTCCAGACCGCAGGCTCCCCCAGCCTCAGGTTGCTGGAGCTGTCACATGACTGCATCCTGCCT |
| | GCCAGGGCTGCAAAGCAAGGTCTTGCTTCTATCTGGGGGACGCTGCTCGAGAGAGGCCGAGAGGCCGC |
| | AGAACATGCCAGGTGTCCTCACTCACCCCACCCTCCCCAATTCCAGCCCTTTGCCCTCTCGGTCAGGG |
| | TTGACCAGGCCAAGGGCTAGCAT |

| | | |
|---|---|---|
| ORF Start: ATG at 330 | | ORF Stop: TGA at 1485 |
| SEQ ID NO:24 | 385 aa | MW at 42653.5kD |

| | |
|---|---|
| NOV3c, | MKDEPRSTNLFMKLDSVFIWKEPFGLVLIIAPWNYPLNLTLVLLVGALAAGNCVVLKPSEISQGTEKV |
| CG118051-03 | |
| Protein | LAEVLPQYLDQSCFAVVLGGPQETGQLLEHKLDYIFFTGSPRVGKIVMTAATKHLTPVTLELGGKNPC |
| Sequence | |
| | YVDDNCDPQTVANRVAWFCYFNAGQTCVAPDYVLCSPEMQERLLPALQSTITRFYGDDPQSSPNLGRI |
| | INQKQFQRLRALLGCGRVAIGGQSNESDRYIAPTVLVDVQETEPVMQEEIFGPILPIVNVQSVDEAIK |
| | FINRQEKPLALYAFSNSSQVVNQMLERTSSGSFGGNEGFTYISLLSVPFGGVGHSGMGRYHGKFTFDT |
| | FSHHRTCPLAPSGLEKLKEIRYPPYTDWNQQLLRWGMGSQSCTLL |

Sequence comparison of the above protein sequences yields the following sequence relationships shown in Table 3B.

TABLE 3B
Comparison of NOV3a against NOV3b and NOV3c.

| | NOV3a Residues/ | Identities/Similarities |
|---|---|---|
| Protein Sequence | Match Residues | for the Matched Region |
| NOV3b | 1 . . . 385 | 331/385 (85%) |
| | 1 . . . 343 | 331/385 (85%) |
| NOV3c | 1 . . . 385 | 363/385 (94%) |
| | 1 . . . 385 | 363/385 (94%) |

Further analysis of the NOV3a protein yielded the following properties shown in Table 3C.

TABLE 3C
Protein Sequence Properties NOV3a

| | |
|---|---|
| PSort analysis: | 0.7900 probability located in plasma membrane; |
| | 0.3000 probability located in Golgi body; |
| | 0.2000 probability located in endoplasmic |
| | reticulum (membrane); |
| | 0.1743 probability located in microbody (peroxisome) |
| SignalP analysis: | Cleavage site between residues 54 and 55 |

A search of the NOV3a protein against the Geneseq database, a proprietary database that contains sequences published in patents and patent publication, yielded several homologous proteins shown in Table 3D.

TABLE 3D
Geneseq Results for NOV3a

| | | NOV3a | Identities/ | |
|---|---|---|---|---|
| | | Residues/ | Similarities | |
| Geneseq | Protein/Organism/Length | Match | for the Matched | Expect |
| Identifier | [Patent #, Date] | Residues | Region | Value |
| AAB58156 | Lung cancer associated | 1 . . . 353 | 325/353 (92%) | 0.0 |
| | polypeptide sequence SEQ ID | 62 . . . 414 | 337/353 (95%) | |
| | 494 - Homo sapiens, 430 aa. | | | |
| | [WO200055180-A2, | | | |
| | 21-SEP-2000] | | | |
| ABB66868 | Drosophila melanogaster | 14 . . . 309 | 158/296 (53%) | 3e−94 |
| | polypeptide SEQ ID NO | 95 . . . 390 | 212/296 (71%) | |
| | 27396 - Drosophila | | | |
| | melanogaster, 561 aa. | | | |
| | [WO200171042-A2, | | | |
| | 27-SEP-2001] | | | |
| ABB65492 | Drosophila melanogaster | 14 . . . 309 | 158/296 (53%) | 3e−94 |
| | polypeptide SEQ ID NO | 95 . . . 390 | 212/296 (71%) | |
| | 23268 - Drosophila | | | |
| | melanogaster, 561 aa. | | | |
| | [WO200171042-A2, | | | |
| | 27-SEP-2001] | | | |
| ABP39856 | Staphylococcus epidermidis | 2 . . . 365 | 157/366 (42%) | 1e−85 |
| | ORF amino acid sequence | 88 . . . 451 | 235/366 (63%) | |
| | SEQ ID NO: 4701 - | | | |
| | Staphylococcus epidermidis, | | | |
| | 464 aa. [US6380370-B1, | | | |
| | 30-APR-2002] | | | |
| AAG82730 | S. epidermidis open reading | 2 . . . 365 | 157/366 (42%) | 1e−85 |
| | frame protein sequence SEQ | 83 . . . 446 | 235/366 (63%) | |
| | ID NO: 2554 - | | | |
| | Staphylococcus | | | |
| | epidermidis, 459 aa. | | | |
| | [WO200134809-A2, | | | |
| | 17-MAY-2001] | | | |

In a BLAST search of public sequence datbases, the NOV3a protein was found to have homology to the proteins shown in the BLASTP data in Table 3E.

TABLE 3E
Public BLASTP Results for NOV3a

| | | NOV3a | | |
|---|---|---|---|---|
| Protein | | Residues/ | Identities/ | |
| Accession | | Match | Similarities for the | Expect |
| Number | Protein/Organism/Length | Residues | Matched Portion | Value |
| P48448 | Aldehyde dehydrogenase 8 | 1 . . . 385 | 385/385 (100%) | 0.0 |
| | (EC 1.2.1.5) - Homo sapiens | 1 . . . 385 | 385/385 (100%) | |
| | (Human), 385 aa. | | | |
| BAC03897 | CDNA FLJ35145 fis, clone | 1 . . . 385 | 380/385 (98%) | 0.0 |
| | PLACE6009853, highly | 1 . . . 385 | 381/385 (98%) | |
| | similar to ALDEHYDE | | | |
| | DEHYDROGENASE 8 (BC | | | |
| | 1.2.1.5) - Homo sapiens | | | |
| | (Human), 385 aa. | | | |
| P43353 | Aldehyde dehydrogenase 7 | 1 . . . 385 | 321/387 (82%) | 0.0 |
| | (EC 1.2.1.5) - Homo sapiens | 82 . . . 468 | 345/387 (88%) | |
| | (Human), 468 aa. | | | |
| AAH33099 | Similar to aldehyde | 13. . . 385 | 315/375 (84%) | 0.0 |
| | dehydrogenase 3 family, | 57 . . . 431 | 339/375 (90%) | |
| | member B1 - Homo sapiens | | | |
| | (Human), 431 aa. | | | |
| Q8VHW0 | Aldehyde dehydrogenase | 1 . . . 385 | 295/387 (76%) | e−174 |
| | ALDH3B1 (EC 1.2.1.3) - | 63 . . . 449 | 336/387 (86%) | |
| | Mus musculus (Mouse), 449 | | | |
| | aa (fragment). | | | |

PFam analysis predicts that the NOV3a protein contains the domains shown in the Table 3F.

TABLE 3F
Domain Analysis of NOV3a

| | | Identities/ | |
|---|---|---|---|
| | NOV3a | Similarities | |
| Pfam | Match | for the Matched | Expect |
| Domain | Region | Region | Value |
| aldedh | 1 . . . 351 | 129/492 (26%) | 1.1e−103 |
| | | 299/492 (61%) | |

### Example 4.

The NOV4 clone was analyzed, and the nucleotide and encoded polypeptide sequences are shown in Table 4A.

TABLE 4A
NOV4 Sequence Analysis

| | |
|---|---|
| SEQ ID NO:25 | 1636 bp |

| | |
|---|---|
| NOV4a, | CCAGGAGCCCCAGTTACCGGGAGAGGCTGTGTCAAAGGCGCCATGAGCAAGATCAGCGAGGCCGTGAA |
| CG120277-01 | |
| DNA Sequence | GCGCGCCCGCGCCGCCTTCAGCTCGGGCAGGACCCGTCCGCTGCAGTTCCGATTCCAGCAGCTGGAGG |
| | CGCTGCAGCGCCTGATCCAGGAGCAGGAGCAGGAGCTGGTGGGCGCGCTGGCCGCAGACCTGCACAAG |
| | AATGAATGGAACGCCTACTATGAGGAGGTGGTGTACGTCCTAGAGGAGATCGAGTACATGATCCAGAA |
| | GCTCCCTGAGTGGGCCGCGGATGAGCCCGTGGAGAAGACGCCCCAGACTCAGCAGGACGAGCTCTACA |
| | TCCACTCGGAGCCACTGGGCGTGGTCCTCGTCATTGGCACCTGGAACTACCCCTTCAACCTCACCATC |
| | CAGCCCATGGTGGGCGCCATCGCTGCAGGGAACGCAGTGGTCCTCAAGCCCTCGGAGCTGAGTGAGAA |
| | CATGGCGAGCCTGCTGGCTACCATCATCCCCCAGTACCTGGACAAGGATCTGTACCCAGTAATCAATG |
| | GGGGTGTCCCTGAGACCACGGAGCTGCTCAAGGAGAGGTTCGACCATATCCTGTACACGGGCAGCACG |
| | GGGGTGGGGAAGATCATCATGACGGCTGCTGCCAAGCACCTGACCCCTGTCACGCTGGAGCTGGGAGG |
| | GAAGAGTCCCTGCTACGTGGACAAGAACTGTGACCTGGACGTGGCCTGCCGACGCATCGCCTGGGGGA |
| | AATTCATGAACAGTGGCCAGACCTGCGTGGCCCCAGACTACATCCTCTGTGACCCCTCGATCCAGAAC |
| | CAAATTGTGGAGAAGCTCAAGAAGTCACTGAAAGAGTTCTACGGGGAAGATGCTAAGAAATCCCGGGA |
| | CTATGGAAGAATCATTAGTGCCCGGCACTTCCAGAGGGTGATGGGCCTGATTGAGGGCCAGAAGGTGG |
| | CTTATGGGGGCACCGGGGATGCCGCCACTCGCTACATAGCCCCCACCATCCTCACGGACGTGGACCCC |
| | CAGTCCCCGGTGATGCAAGAGGAGATCTTCGGGCCTGTGCTGCCCATCGTGTGCGTGCGCAGCCTGGA |
| | GGAGGCCATCCAGTTCATCAACCAGCGTGAGAAGCCCCTGGCCCTCTACATGTTCTCCAGCAACGACA |
| | AGGTGATTAAGAAGATGATTGCAGAGACATCCAGTGGTGGGGTGGCGGCCAACGATGTCATCGTCCAC |
| | ATCACCTTGCACTCTCTGCCCTTCGGGGGCGTGGGGAACAGCGGCATGGGATCCTACCATGGCAAGAA |
| | GAGCTTCGAGACTTTCTCTCACCGCCGCTCTTGCCTGGTGAGGCCTCTGATGAATGATGAAGGCCTGA |
| | AGGTCAGATACCCCCCGAGCCCGGCCAAGATGACCCAGCACTGAGGAGGGGTTGCTCCGCCTGGCCTG |
| | GCCATACTGTGTCCCATCGGAGTGCGGACCACCCTCACTGGCTCTCCTGGCCCTGGAGAATCGCTCCT |
| | GCAGCCCCAGCCCAGCCCCACTCCTCTGCTGACCTGCTGACCTGTGCACACCCCACTCCCACATGGGC |
| | CCAGGCCTCACCATTCCAAGTCTCCACCCCTTTCTAGACCAATAAAGAGACAAATACAATTTTCTAAC |
| | TCGG |

| | | |
|---|---|---|
| ORF Start: ATG at 43 | | ORF Stop: TGA at 1402 |
| SEQ ID NO:26 | 453 aa | MW at 50412.5 kD |

| | |
|---|---|
| NOV4a, | MSKISEAVKRARAAFSSGRTRPLQFRFQQLEALQRLIQEQEQELVGALAADLHKNEWNAYYEEVVYVL |
| CG120277-01 | |
| Protein | EEIEYMIQKLPEWAADEPVEKTPQTQQDELYIHSEPLGVVLVIGTWNYPFNLTIQPMVGAIAAGNAVV |
| Sequence | |
| | LKPSELSENMASLLATIIPQYLDKDLYPVINGGVPETTELLKERFDHILYTGSTGVGKIIMTAAAKHL |
| | TPVTLELGGKSPCYVDKNCDLDVACRRIAWGKFMNSGQTCVAPDYILCDPSIQNQIVEKLKKSLKEFY |
| | GEDAKKSRDYGRIISARHFQRVMGLIEGQKVAYGGTGDAATRYIAPTILTDVDPQSPVMQEEIFGPVL |
| | PIVCVRSLEEAIQFINQREKPLALYMFSSNDKVIKKMIAETSSGGVAANDVIVHITLHSLPFGGVGNS |
| | GMGSYHGKKSFETFSHRRSCLVRPLMNDEGLKVRYPPSPAKMTQH |

| | |
|---|---|
| SEQ ID NO:27 | 1554 bp |

| | |
|---|---|
| NOV4b, | GAGCCCCAGTTACCGGGAGAGGCTGTGTCAAAGGCGCCATGAGCAAGATCAGCGAGGCCGTGAAGCG |
| CG120277-02 | |
| DNA Sequence | CGCCCGCGCCGCCTTCAGCTCGGGCAGGACCCGTCCGCTGCAGTTCCGGATCCAGCAGCTGGAGGCG |
| | CTGCAGCGCCTGATCCAGGAGCAGGAGCAGGAGCTGGTGGGCGCGCTGGCCGCAGACCTGCACAAGA |
| | ATGAATGGAACGCCTACTATGAGGAGGTGGTGTACGTCCTAGAGGAGATCGAGTACATGATCCAGAA |
| | GCTCCCTGAGTGGGCCGCGGATGAGCCCGTGGAGAAGACGCCCCAGACTCAGCAGGACGAGCTCTAC |
| | ATCCACTCGGAGCCACTGGGCGTGGTCCTCGTCATTGGCACCTGGAACTACCCCTTCAACCTCACCA |
| | TCCAGCCCATGGTGGGCGCCATCGCTGCAGGGAACGCAGTGGTCCTCAAGCCCTCGGAGCTGAGTGA |
| | GAACATGGCGAGCCTGCTGGCTACCATCATCCCCCAGTACCTGGACAAGGATCTGTACCCAGTAATC |
| | AATGGGGGTGTCCCTGAGACCACGGAGCTGCTCAAGGAGAGGTTCGACCATATCCTGTACACGGGCA |
| | GCACGGGGGTGGGGAAGATCATCATGACGGCTGCTGCCAAGCACCTGACCCCTGTCACGCTGGAGCT |
| | GGGAGGGAAGAGTCCCTGCTACGTGGACAAGAACTGTGACCTGGACGTGGCCTGCCGACGCATCGCC |
| | TGGGGGAAATTCATGAACAGTGGCCAGACCTGCGTGGCCCCAGACTACATCCTCTGTGACCCCTCGA |
| | TCCAGAACCAAATTGTGGAGAAGCTCAAGAAGTCACTGAAAGAGTTCTACGGGGAAGATGCTAAGAA |
| | ATCCCGGGACTATGGAAGAATCATTAGTGCCCGGCACTTCCAGAGGGTGATGGGCCTGATTGAGGGC |
| | CAGAAGGTGGCTTATGGGGGCACCGGGGATGCCGCCACTCGCTACATAGCCCCCACCATCCTCACGG |
| | ACGTGGACCCCCAGTCCCCGGTGATGCAAGAGGAGATCTTCGGGCCTGTGCTGCCCATCGTGTGCGT |
| | GCGCAGCCTGGAGGAGGCCATCCAGTTCATCAACCAGCGTGAGAAGCCCCTGGCCCTCTACATGTTC |
| | TCCAGCAACGACAAGGTGATTAAGAAGATGATTGCAGAGACATCCAGTGGTGGGGTGGCGGCCAACG |
| | ATGTCATCGTCCACATCACCTTGCACTCTCTGCCCTTCGGGGGCGTGGGGAACAGCGGCATGGTGAG |
| | GCCTCTGATGAATGATGAAGGCCTGAAGGTCAGATACCCCCCGAGCCCGGCCAAGATGACCCAGCAC |
| | TGAGGAGGGGTTGCTCCGTCTGGCCTGGCCATACTGTGTCCCATCGGAGTGCGGACCACCCTCACTG |
| | GCTCTCCTGGCCCTGGGAGAATCGCTCCTGCAGCCCCAGCCCAGCCCCACTCCTCTGCTGACCTGCT |
| | GACCTGTGCACACCCCACTCCCACATGGGCCCAGGCCTCACCATTCCAAGTCTCCACCCCTTTCTAG |
| | ACCAATAAAGAGA |

| | | |
|---|---|---|
| ORF Start: ATG at 39 | | ORF Stop: TGA at 1341 |
| SEQ ID NO:28 | 434 aa | MW at 48169.0 kD |

| | |
|---|---|
| NOV4b, | MSKISEAVKRARAAFSSGRTRPLQFRIQQLEALQRLIQEQEQELVGALAADLHKNEWNAYYEEVVYV |
| CG120277-02 | |
| Protein | LEEIEYMIQKLPEWAADEPVEKTPQTQQDELYIHSEPLGVVLVIGTWNYPFNLTIQPMVGAIAAGNA |
| Sequence | |
| | VVLKPSELSENMASLLATIIPQYLDKDLYPVINGGVPETTELLKERFDHILYTGSTGVGKIIMTAAA |
| | KHLTPVTLELGGKSPCYVDKNCDLDVACRRIAWGKFMNSGQTCVAPDYILCDPSIQNQIVEKLKKSL |
| | KEFYGEDAKKSRDYGRIISARHFQRVMGLIEGQKVAYGGTGDAATRYIAPTILTDVDPQSPVMQEEI |
| | FGPVLPIVCVRSLEEAIQFINQREKPLALYMFSSNDKVIKKMIAETSSGGVAANDVIVHITLHSLPF |
| | GGVGNSGMVRPLMNDEGLKVRYPPSPAKMTQH |

Sequence comparison of the above protein sequences yields the following sequence relationships shown in Table 4B.

TABLE 4B
Comparison of NOV4a against NOV4b.

| | | Identities/ |
|---|---|---|
| | | Similarities |
| Protein | NOV4a Residues/ | for the Matched |
| Sequence | Match Residues | Region |
| NOV4b | 1 . . . 453 | 401/453 (88%) |
| | 1 . . . 434 | 401/453 (88%) |

Further analysis of the NOV4a protein yielded the following properties shown in Table 4C.

TABLE 4C
Protein Sequence Properties NOV4a

| | |
|---|---|
| PSort analysis: | 0.7636 probability located in mitochondrial matrix |
| | space; 0.4422 probability located in mitochondrial |
| | inner membrane; 0.4422 probability located in |
| | mitochondrial intermembrane space; 0.4422 |
| | probability located in mitochondrial |
| | outer membrane |
| SignalP analysis: | No Known Signal Sequence Predicted |

A search of the NOV4a protein against the Geneseq database, a proprietary database that contains sequences published in patents and patent publication, yielded several homologous proteins shown in Table 4D.

TABLE 4D
Geneseq Results for NOV4a

| | | | Identities/ | |
|---|---|---|---|---|
| | | NOV4a | Similarities for | |
| Geneseq | Protein/Organism/Length | Residues/Match | the Matched | Expect |
| Identifier | [Patent #, Date] | Residues | Region | Value |
| AAB58156 | Lung cancer associated | 48 . . . 431 | 208/384 (54%) | e−124 |
| | polypeptide sequence SEQ ID | 28 . . . 411 | 277/384 (71%) | |
| | 494 - Homo sapiens, 430 aa. | | | |
| | [WO200055180-A2, | | | |
| | 21 SEP. 2000] | | | |
| ABB66868 | Drosophila melanogaster | 1 . . . 394 | 199/394 (50%) | e−115 |
| | polypeptide SEQ ID NO | 1 . . . 394 | 270/394 (68%) | |
| | 27396 - Drosophila | | | |
| | melanogaster, 561 aa. | | | |
| | [WO200171042-A2, | | | |
| | 27 SEP. 2001] | | | |
| ABB65492 | Drosophila melanogaster | 1 . . . 394 | 199/394 (50%) | e−115 |
| | polypeptide SEQ ID NO | 1 . . . 394 | 270/394 (68%) | |
| | 23268 - Drosophila | | | |
| | melanogaster, 561 aa. | | | |
| | [WO200171042-A2, | | | |
| | 27 SEP. 2001] | | | |
| AAG21988 | Arabidopsis thaliana protein | 2 . . . 445 | 210/449 (46%) | e−112 |
| | fragment SEQ ID NO: 24747 - | 10 . . . 456 | 288/449 (63%) | |
| | Arabidopsis thaliana, 484 | | | |
| | aa. [EP1033405-A2, | | | |
| | 06 SEP. 2000] | | | |
| AAG11789 | Arabidopsis thaliana protein | 2 . . . 445 | 210/449 (46%) | e−112 |
| | fragment SEQ ID NO: 10644 - | 10 . . . 456 | 288/449 (63%) | |
| | Arabidopsis thaliana, 484 | | | |
| | aa. [EP1033405-A2, | | | |
| | 06 SEP. 2000] | | | |

In a BLAST search of public sequence datbases, the NOV4a protein was found to have homology to the proteins shown in the BLASTP data in Table 4E.

TABLE 4E
Public BLASTP Results for NOV4a

| | | | Identities/ | |
|---|---|---|---|---|
| Protein | | | Similarities | |
| Accession | | NOV4a Residues/ | for the | |
| Number | Protein/Organism/Length | Match Residues | Matched Portion | Expect Value |
| P30838 | Aldehyde dehydrogenase, | 1 . . . 453 | 453/453 (100%) | 0.0 |
| | dimeric NADP-preferring (EC | 1 . . . 453 | 453/453 (100%) | |
| | 1.2.1.5) (ALDH class 3) | | | |
| | (ALDHIII) - Homo sapiens | | | |
| | (Human), 453 aa. | | | |
| Q9BT37 | Aldehyde dehydrogenase 3 | 1 . . . 453 | 452/453 (99%) | 0.0 |
| | (Aldehyde dehydrogenase 3 | 1 . . . 453 | 452/453 (99%) | |
| | family, member Al) - Homo | | | |
| | sapiens (Human), 453 aa. | | | |
| A42584 | aldehyde dehydrogenase | 1 . . . 453 | 450/453 (99%) | 0.0 |
| | (NAD(P)+) (EC 1.2. 1.5) 3 - | 1 . . . 453 | 451/453 (99%) | |
| | human, 453 aa. | | | |
| A30149 | aldehyde dehydrogenase | 1 . . . 453 | 370/453 (81%) | 0.0 |
| | (NADP+) (EC 1.2.1.4) 3, | 1 . . . 453 | 415/453 (90%) | |
| | tumor-associated [similarity] - | | | |
| | rat, 453 aa. | | | |
| P11883 | Aldehyde dehydrogenase, | 2 . . . 453 | 369/452 (81%) | 0.0 |
| | dimeric NADP-preferring (EC | 1 . . . 452 | 414/452 (90%) | |
| | 1.2.1.5) (ALDH class 3) | | | |
| | (Tumor-associated aldehyde | | | |
| | dehydrogenase) | | | |
| | (HTC-ALDH) - Rattus | | | |
| | norvegicus (Rat), 452 aa. | | | |

PFam analysis predicts that the NOV4a protein contains the domains shown in the Table 4F.

TABLE 4F
Domain Analysis of NOV4a

| | | Identities/ | |
|---|---|---|---|
| | NOV4a | Similarities | |
| Pfam | Match | for the | Expect |
| Domain | Region | Matched Region | Value |
| aldedh | 1 . . . 432 | 182/492 (37%) | 7.4e−206 |
| | | 401/492 (82%) | |

### Example 5

The NOV5 clone was analyzed, and the nucleotide and encoded polypeptide sequences are shown in Table 5A.

TABLE 5A
NOV5 Sequence Analysis

| | |
|---|---|
| SEQ ID NO: 29 | 2316 bp |

| | |
|---|---|
| NOV5a, | GCCACGAAGGCCACAGACGCCTTCCCCCTTGGACTCTCATTCCCTTTTCCACGGAGCCCCGCGCTTTC |
| GC140468-01 | |
| DNA Sequence | GTGAGCCCCCTCGAGGAACCTGGTCTCCGCATCCAGTTACCACCTCCTGCCTCAGAGGCCATCTGAGC |
| | CCTTCGCACCTCGCCCCTCAGTCCCCCCTTGCCCCCCCGCGGAGATCGCCTCGCTCCCTCCCGCCCCC |
| | CCATCATCCCTTCCCTCGCAGTTCCCCTGTCCTGAGGGGAGCCCCGCCACGGCAGCGACAGCGGGCAG |
| | GAGGGAGAAAGTGAAGGTTGGGCGACACTTGGCCTCACTCCCGGCTAGGCGCACCCACGGGGAGGAGA |
| | GGAGGAGCCGAGAGAGCTGAGCAGCGCGGAAGTAGCTGCTGCTGGTGGTGACAATGTCAAATAACGGC |
| | CTAGACATTCAAGACAAACCCCCAGCCCCTCCGATGAGAAATACCAGCACTATGATTGGAGTCGGCAG |
| | CAAAGATGCTGGAACCCTAAACCATGGTTCTAAACCTCTGCCTCCAAACCCAGAGGAGAAGAAAAAGA |
| | AGGACCGATTTTACCGATCCATTTTACCTGGAGATAAAACAAATAAAAAGAAAGAGAAAGAGCGGCCA |
| | GAGATTTCTCTCCCTTCAGATTTTGAACACACAATTCATGTCCGTTTTGATGCTGTCACAGGGGAGTT |
| | TACGGGAATGCCAGAGCAGTGGGCCCGCTTGCTTCAGACATCAAATATCACTAAGTCGGAGCAGAAGA |
| | AAAACCCGCAGGCTGTTCTGGATGTGTTGGAGTTTTACAACTCGAAGAAGACATCCAACAGCCAGAAA |
| | TACATGAGCTTTACAGATAAGTCAGCTGAGGATTACAATTCTTCTAATGCCTTGAATGTGAAGGCTGT |
| | GTCTGAGACTCCTGCAGTGCCACCAGTTTCAGAAGATGAGGATGATGATGATGATGATGCTACCCCAC |
| | CACCAGTGATTGCTCCACGCCCAGAGCACACAAAATCTGTATACACACGGTCTGTGATTGAACCACTT |
| | CCTGTCACTCCAACTCGGGACGTGGCTACATCTCCCATTTCACCTACTGAAAATAACACCACTCCACC |
| | AGATGCTTTGACCCGGAATACTGAGAAGCAGAAGAAGAAGCCTAAAATGTCTGATGAGGAGATCTTGG |
| | AGAAATTACGAAGCATAGTGAGTGTGGGCGATCCTAAGAAGAAATATACACGGTTTGAGAAGATTGGA |
| | CAAGGTGCTTCAGGCACCGTGTACACAGCAATGGATGTGGCCACAGGACAGGAGGTGGCCATTAAGCA |
| | GATGAATCTTCAGCAGCAGCCCAAGAAAGAGCTGATTATTAATGAGATCCTGGTCATGAGGGAAAACA |
| | AGAACCCAAACATTGTGAATTACTTGGACAGTTACCTCGTGGGAGATGAGCTGTGGGTTGTTATGGAA |
| | TACTTGGCTGGAGGCTCCTTGACAGATGTGGTGACAGAAACTTGCATGGATGAAGGCCAAATTGCAGC |
| | TGTGTGCCGTGAGTGTCTGCAGGCTCTGGAGTTCTTGCATTCGAACCAGGTCATTCACAGAGACATCA |
| | AGAGTGACAATATTCTGTTGGGAATGGATGGCTCTGTCAAGCTAACTGACTTTGGATTCTGTGCACAG |
| | ATAACCCCAGAGCAGAGCAAACGGAGCACCATGGTAGGAACCCCATACTGGATGGCACCAGAGGTTGT |
| | GACACGAAAGGCCTATGGGCCCAAGGTTGACATCTGGTCCCTGGGCATCATGGCCATCGAAATGATTG |
| | AAGGGGAGCCTCCATACCTCAATGAAAACCCTCTGAGAGCCTTGTACCTCATTGCCACCAATGGGACC |
| | CCAGAACTTCAGAACCCAGAGAAGCTGTCAGCTATCTTCCGGGACTTTCTGAACCGCTGTCTCGATAT |
| | GGATGTGGAGAAGAGAGGTTCAGCTAAAGAGCTGCTACAGCATCAATTCCTGAAGATTGCCAAGCCCC |
| | TCTCCAGCCTCACTCCACTGATTGCTGCAGCTAAGGAGGCAACAAAGAACAATCACTAAAACCACACT |
| | CACCCCAGCCTCATTGTGCCAAGCTCTGTGAGATAAATGCACATTTCAGAAATTCCAACTCCTGATGC |
| | CCTCTTCTCCTTGCCTTGCTTCTCCCATTTCCTGATCTAGCACTCCTCAAGACTTTGATCCTTGGAAA |
| | CCGTGTGTCCAGCATTGAAGAGAACTGCAACTGAATGACTAATCAGATGATGGCCATTTCTAAATAAG |
| | GAATTTCCTCCCAATTCATGGATATGAGGGTGGTTTATGATTAAGGGTTTATATAAATAAATGTTTCT |
| | AGTC |

| | | |
|---|---|---|
| ORF Start: ATG at 394 | | ORF Stop: TAA at 2029 |
| SEQ ID NO: 30 | 545 aa | MW at 60660.3kD |

| | |
|---|---|
| NOV5a, | MSNNGLDIQDKPPAPPMRNTSTMIGVGSKDAGTLNHGSKPLPPNPEEKKKKDRFYRSILPGDKTNKKK |
| CG140468-01 | |
| Protein | EKERPEISLPSDFEHTIHVGFDAVTGEFTGMPEQWARLLQTSNITYSEQKKNPQAVLDVLEFYNSKKT |
| Sequence | |
| | SNSQKYMSFTDKSAEDYNSSNALNVKAVSETPAVPPVSEDEDDDDDDATPPPVIAPRPEHTKSVYTRS |
| | VIEPLPVTPTRDVATSPISPTENNTTPPDALTRNTEKQKKKPKMSDEEILEKLRSIVSVGDPKKKYTR |
| | FEKIGQGASGTVYTAMDVATGQEVAIKQMNLQQQPKKELIINEILVMRENKNPNIVNYLDSYLVGDEL |
| | WVVMEYLAGGSLTDVVTETCMDEGQIAAVCRECLQALEFLHSNQVIHRDIKSDNILLGMDGSVKLTDF |
| | GFCAQITPEQSKRSTMVGTPYWMAPEVVTRKAYGPKVDIWSLGIMAIEMIEGEPPYLNENPLRALYLI |
| | ATNGTPELQNPEKLSAIFRDFLNRCLDMDVEKRGSAKELLQHQFLKIAKPLSSLTPLIAAAKEATKNN |
| | H |

| | |
|---|---|
| SEQ ID NO: 31 | 957 bp |

| | |
|---|---|
| NOV5b, | GACAATGTCAAATAACGGCCTAGACATTCAAGACAAACCCCCAGCCCCTCCGATGAGAAATACCAGC |
| CG140468-02 | |
| DNA Sequence | ACTATGATTGGAGCCGGCAGCAAAGATGCTGGAACCCTAAACCATGGTTCTAAACCTCTGCCTCCAA |
| | ACCCAGAGGAGAAGAAAAAGAAGGACCGATTTTACCGATCCATTTTACCTGGAGATAAAACAAATAA |
| | AAAGAAAGAGAAAGAGCGGCCAGAGATTTCTCTCCCTTCAGATTTTGAACACACAATTCATGTCGGT |
| | TTTGATGCTGTCACAGGGGAGTTTACGGGAATGCCAGAGCAGTGGGCCCGCTTGCTTCAGACATCAA |
| | ATATCACTAAGTCGGAGCAGAAGAAAAACCCGCAGGCTGTTCTGGATGTGTTGGAGTTTTACAACTC |
| | GAAGAAGACATCCAACAGCCAGAAATACATGAGCTTTACAGATAAGTCAGCTGAGGATTACAATTCT |
| | TCTAATGCCTTGAATGTGAAGGCTGTGTCTGAGACTCCTGCAGTGCCACCAGTTTCAGAAGATGAGG |
| | ATGATGATGATGATGATGCTACCCCACCACCAGTGATTGCTCCACGCCCAGAGCACACAAAATCTGT |
| | ATACACACGGTCTGTGATTGAACCACTTCCTGTCACTCCAACTCGGGACGTGGCTACATCTCCCATT |
| | TCACCTACTGAAAATAACACCACTCCACCAGATGCTTTGACCCGGAATACTGAGAAGCAGAAGAAGA |
| | AGCCTAAAATGTCTGATGAGGAGATCTTGGAGAAATTACGAAGCATAGTGAGTGTGGGCGATCCTAA |
| | GAAGAAATATACACGGTTTGAGAAGATTGCCAAGCCCCTCTCCAGCCTCACTCCACTGATTGCTGCA |
| | GCTAAGGAGGCAACAAAGAACAATCACTAAAACCACACTCACCCCAGCCTCATTGTGCCAAGCCTTC |
| | TGTGAGATAAATGCACATT |

| | | |
|---|---|---|
| ORF Start: ATG at 5 | | ORF Stop: TAA at 899 |
| SEQ ID NO: 32 | 298 aa | MW at 32989.7kD |

| | |
|---|---|
| NOV5b, | MSNNGLDIQDKPPAPPMRNTSTMIGAGSKDAGTLNHGSKPLPPNPEEKKKKDRFYRSILPGDKTNKK |
| CG140468-02 | |
| Protein | KEKERPEISLPSDFEHTIHVGFDAVTGEFTGMPEQWARLLQTSNITKSEQKKNPQAVLDVLEFYNSK |
| Sequence | |
| | KTSNSQKYMSFTDKSAEDYNSSNALNVKAVSETPAVPPVSEDEDDDDDDATPPPVIAPRPEHTKSVY |
| | TRSVIEPLPVTPTRDVATSPISPTENNTTPPDALTRNTEKQKKKPKMSDEEILEKLRSIVSVGDPKK |
| | KYTRFEKIAKPLSSLTPLIAAAKEATKNNH |

Sequence comparison of the above protein sequences yields the following sequence relationships shown in Table 5B.

TABLE 5B
Comparison of NOV5a against NOV5b.

| | | Identities/ |
|---|---|---|
| | | Similarities |
| | NOV5a Residues/ | for the |
| Protein Sequence | Match Residues | Matched Region |
| NOV5b | 1 . . . 281 | 238/281 (84%) |
| | 1 . . . 281 | 239/281 (84%) |

Further analysis of the NOVSa protein yielded the following properties shown in Table 5C.

TABLE 5C
Protein Sequence Properties NOV5a

| | |
|---|---|
| PSort analysis: | 0.7000 probability located in nucleus; 0.3000 |
| | probability located in microbody (peroxisome); |
| | 0.1000 probability located in mitochondrial |
| | matrix space; 0.1000 probability located in |
| | lysosome (lumen) |
| SignalP analysis: | No Known Signal Sequence Predicted |

A search of the NOV5a protein against the Geneseq database, a proprietary database that contains sequences published in patents and patent publication, yielded several homologous proteins shown in Table 5D.

TABLE 5D
Geneseq Results for NOV5a

| | | | Identities/ | |
|---|---|---|---|---|
| | | | Similarities | |
| Geneseq | Protein/Organism/Length | NOV5a Residues/ | for the Matched | Expect |
| Identifier | [Patent #, Date] | Match Residues | Region | Value |
| AAB03968 | p-21 activated protein kinase | 1 . . . 545 | 544/545 (99%) | 0.0 |
| | (PAK1) - Homo sapiens, 545 | 1 . . . 545 | 545/545 (99%) | |
| | aa. [WO200060062-A2, | | | |
| | 12 OCT. 2000] | | | |
| AAY55958 | Human STE20-related | 1 . . . 545 | 541/545 (99%) | 0.0 |
| | protein kinase PAK1_h - | 1 . . . 545 | 542/545 (99%) | |
| | Homo sapiens, 545 aa. | | | |
| | [W09953036-A2, | | | |
| | 21 OCT. 1999] | | | |
| ABG30251 | Novel human diagnostic | 1 . . . 542 | 474/556 (85%) | 0.0 |
| | protein #30242 - Homo | 7 . . . 557 | 500/556 (89%) | |
| | sapiens, 587 aa. | | | |
| | [WO200175067-A2, | | | |
| | 11 OCT. 2001] | | | |
| AAW72757 | Human doublin - Homo | 3 . . . 544 | 444/552 (80%) | 0.0 |
| | sapiens, 544 aa. | 2 . . . 542 | 489/552 (88%) | |
| | [WO9840495-A1, | | | |
| | 17 SEP. 1998] | | | |
| ABB57290 | Mouse ischaemic condition | 3 . . . 544 | 441/552 (79%) | 0.0 |
| | related protein sequence SEQ | 2 . . . 542 | 483/552 (86%) | |
| | ID NO: 817 - Mus musculus, | | | |
| | 544 aa. [WO200188188-A2, | | | |
| | 22 NOV. 2001] | | | |

In a BLAST search of public sequence datbases, the NOV5a protein was found to have homology to the proteins shown in the BLASTP data in Table 5E.

TABLE 5E
Public BLASTP Results for NOV5a

| | | | Identities/ | |
|---|---|---|---|---|
| Protein | | | Similarities | |
| Accession | | NOV5a Residues/ | for the | Expect |
| Number | Protein/Organism/Length | Match Residues | Matched Portion | Value |
| Q13153 | Serine/threonine-protein | 1 . . . 545 | 545/545 (100%) | 0.0 |
| | kinase PAK1 (EC 2.7.1.-) | 1 . . . 545 | 545/545 (100%) | |
| | (p21-activated kinase 1) | | | |
| | (PAK-1) (P65-PAK) | | | |
| | (Alpha-PAK) - Homo sapiens | | | |
| | (Human), 545 aa. | | | |
| P35465 | Serine/threonine-protein | 1 . . . 545 | 537/545 (98%) | 0.0 |
| | kinase PAK1 (EC 2.7.1.-) | 1 . . . 544 | 539/545 (98%) | |
| | (p21-activated kinase 1) | | | |
| | (PAK-1) (P68-PAK) | | | |
| | (Alpha-PAK) (Protein kinase | | | |
| | MUK2) - Rattus norvegicus | | | |
| | (Rat), 544 aa. | | | |
| S40482 | serine/threonine-specific | 1 . . . 545 | 534/545 (97%) | 0.0 |
| | protein kinase (EC 2.7.1.-) - | 1 . . . 544 | 537/545 (97%) | |
| | rat, 544 aa. | | | |
| O88643 | Serine/threonine-protein | 1 . . . 545 | 530/545 (97%) | 0.0 |
| | kinase PAK1 (EC 2.7.1.-) | 1 . . . 545 | 537/545 (98%) | |
| | (p21-activated kinase 1) | | | |
| | (PAK-1) (P65-PAK) | | | |
| | (Alpha-PAK) (CDC42/RAC | | | |
| | effector kinase PAK-A) - Mus | | | |
| | musculus (Mouse), 545 aa. | | | |
| O75561 | P21 activated kinase 1B - | 1 . . . 522 | 517/522 (99%) | 0.0 |
| | Homo sapiens (Human), 553 | 1 . . . 522 | 520/522 (99%) | |
| | aa. | | | |

PFam analysis predicts that the NOV5a protein contains the domains shown in the Table 5F.

TABLE 5F
Domain Analysis of NOV5a

| | | Identities/ | |
|---|---|---|---|
| | | Similarities | |
| | NOV5a Match | for the Matched | Expect |
| Pfam Domain | Region | Region | Value |
| PBD | 75 . . . 135 | 37/64 (58%) | 3.4e−34 |
| | | 59/64 (92%) | |
| pkinase | 270 . . . 521 | 94/291 (32%) | 5.7e−90 |
| | | 208/291 (71%) | |

### Example 6

The NOV6 clone was analyzed, and the nucleotide and encoded polypeptide sequences are shown in Table 6A.

TABLE 6A
NOV6 Sequence Analysis

| | |
|---|---|
| SEQ ID NO: 33 | 3255 bp |

| | |
|---|---|
| NOV6a, | GACAGCTTTGGGTGGACCAGTAATGAGGAAATGAGGCAACATGATGTGCAGGAACTGAATCGAATCCT |
| CG142182-01 | |
| DNA Sequence | CTTCAGCGCTTTGGAAACTTCTTTAGTTGGGACCTCCGGTCATGACCTCATCTATCGTCTGTACCATG |
| | GAACCATTGTTAACCAGATTGTTTGTAAAGAATGTAAGAACGTTAGCGAGAGGCAGGAAGACTTCTTA |
| | GATCTAACAGTAGCAGTCAAAAATGTATCCGGTTTGGAAGATGCTCTCTGGAACATGTATGTAGAAGA |
| | GGAAGTTTTTGATTGTGACAACTTGTACCACTGTGGAACTTGTGACAGGCTGGTTAAAGCAGCAAAGT |
| | CGGCCAAATTACGTAAGCTGCCTCCTTTTCTTACTGTTTCATTACTAAGATTTAATTTTGATTTTGTG |
| | AAATGCGAACGCTACAAGGAAACTAGCTGTTATACATTCCCTCTCCGGATTAATCTCAAGCCCTTTTG |
| | TGAACAGAGTGAATTGGATGACTTAGAATATATATATGACCTCTTCTCAGTTATTATACACAAAGGTG |
| | GCTGCTACGGAGGCCATTACCATGTATATATTAAAGATGTTGATCATTTGGGAAACTGGCAGTTTCAA |
| | GAGGAAAAAAGTAAACCAGATGTGAATCTGAAAGATCTCCAGAGTGAAGAAGAGATTGATCATCCACT |
| | GATGATTCTAAAAGCAATCTTATTAGAGGAGGAGAATAATCTAATTCCTGTTGATCAGCTGGGCCAGA |
| | AACTTTTGAAAAAGATAGGAATATCTTGGAACAAGAAGTACAGAAAACAGCATGGACCATTGCGGAAG |
| | TTCTTACAGCTCCATTCTCAGATATTTCTACTCAGTTCAGATGAAAGTACAGTTCGTCTCTTGAAGAA |
| | TAGTTCTCTCCAGGCTGAGTCTGATTTCCAAAGGAATGACCAGCAAATTTTCAAGATGCTTCCTCCAG |
| | AATCCCCAGGTTTAAACAATAGCATCTCCTGTCCCCACTGGTTTGATATAAATGATTCTAAAGTCCAG |
| | CCAATCAGGGAAAAGGATATTGAACAGCAATTTCAGGGTAAAGAAAGTGCCTACATGTTGTTTTATCG |
| | GAAATCCCAGTTGCAGAGACCCCCTGAAGCTCGAGCTAATCCAAGATATGGGGTTCCATGTCATTTAC |
| | TGAATGAAATGGATGCAGCTAACATTGAACTGCAAACCAAAAGGGCAGAATGTGATTCTGCAAACAAT |
| | ACTTTTGAATTGCATCTTCACCTGGGCCCTCAGTATCATTTCTTCAATGGGGCTCTGCACCCAGTAGT |
| | CTCTCAAACAGAAAGCGTGTGGGATTTGACCTTTGATAAAAGAAAAACTTTAGGAGATCTCCGGCAGT |
| | CAATATTTCAGCTGTTAGAATTTTGGGAAGGAGACATGGTTCTTAGTGTTGCAAAGCTTGTACCAGCA |
| | GGACTTCACATTTACCAGTCACTTGGCGGGGATGAACTGACACTGTGTGAAACTGAAATTGCTGATGG |
| | GGAAGACATCTTTGTGTGGAATGGGGTGGAGGTTGGTGGAGTCCACATTCAAACTGGTATTGACTGCG |
| | AACCTCTACTTTTAAATGTTCTTCATCTAGACACAAGCAGTGATGGAGAAAAGTGTTGTCAGGTGATA |
| | GAATCTCCACATGTCTTTCCAGCTAATGCAGAAGTGGGCACTGTCCTCAACGCCTTAGCAATCCCAGC |
| | AGGTGTCATCTTCATCAACAGTGCTGGATGTCCAGGTGGGGAGGGTTGGACGGCCATCCCCAAGGAAG |
| | ACATGAGGAAGACGTTCAGGGAGCAAGGGCTCAGAAATGGAAGCTCAATTTTAATTCAGGATTCTCAT |
| | GATGATAACAGCTTGTTGACCAAGGAAGAGAAATGGGTCACTAGTATGAATGAGATTGACTGGCTCCA |
| | CGTTAAAAATTTATGCCAGTTAGAATCTGAAGAGAAGCAAGTTAAAATATCAGCAACTGTTAACACAA |
| | TGGTGTTTGATATTCGAATTAAAGCCATAAAGGAATTAAAATTAATGAAGGAACTAGCTGACAACAGC |
| | TGTTTGAGACCTATTGATAGAAATGGGAAGCTTCTTTGTCCAGTGCCGGACAGCTATACTTTGAAGGA |
| | AGCAGAATTGAAGATGGGAAGTTCATTGGGACTGTGTCTTGGAAAAGCACCAAGTTCGTCTCAGTTGT |
| | TCCTGTTTTTTGCAATGGGGAGTGACGTTCAACCTGGGACAGAAATGGAAATCGTAGTAGAAGAAACA |
| | ATATCTGTGAGAGATTGTTTAAAGTTAATGCTGAAGAAATCTGGCCTACAAGACTCCTTTATAGGAGA |
| | TGCCTGGCATTTACGAAAAATGGATTGGTGCTATGAAGCTGGAGAGCCTTTATGTGAAGAAGATGCAA |
| | CACTGAAAGAACTTCTGATATGTTCTGGAGATACTTTGCTTTTAATTGAAGGACAACTTCCTCCTCTG |
| | GGTTTCCTGAAGGTGCCCATCTGGTGGTACCAGCTTCAGGGTCCCTCAGGACACTGGGAGAGTCATCA |
| | GGACCAGACCAACTGTACTTCGTCTTGGGGCAGAGTTTGGAGAGCCACTTCCAGCCAAGGTGCTTCTG |
| | GGAACGAGCCTGCGCAAGTTTCTCTCCTCTACTTGGGAGACATAGAGATCTCAGAAGATGCCACGCTG |
| | GCGGAGCTGAAGTCTCAGGCCATGACCTTGCCTCCTTTCCTGGAGTTCGGTGTCCCGTCCCCAGCCCA |
| | CCTCAGAGCCTGGACGGTGGAGAGGAAGCGCCCAGGCAGGCTTTTACGAACTGACCGGCAGCCACTCA |
| | GGGAATATAAACTAGGACGGAGAATTGAGATCTGCTTAGAGCCCCTTCAGAAAGGCGAAAACTTGGGC |
| | CCCCAGGACGTGCTGCTGAGGACACAGGTGCGCATCCCTGGTGAGAGGACCTATGCCCCTGCCCTGGA |
| | CCTGGTGTGGAACGCGGCCCAGGGTGGGACTGCCGGCTCCCTGAGGCAGAGAGTTGCCGATTTCTATT |
| | GTCTTCCCGTGGAGAAGATTGAAATTGCCAAATACTTTCCCGAAAAGTTCGAGTGGCTTCCGATATCT |
| | AGCTGGAACCAACAAATAACCAAGAGGAAAAAAAAAAAAAAACAAGATTATTTGCAAGGGGCACCGTA |
| | TTACTTGAAAGACGGAGATACTATTGGTGTTAAGGTAAGTTGTTTAACAGCAAATTTACCACTTTGAG |
| | AAGACACGAGGGTCACATGATTTTATAGAGACGTTTTATTGAATCTTCAAGACACAGAT |

| | | |
|---|---|---|
| ORF Start: ATG at 31 | | ORF Stop: TGA at 3193 |
| SEQ ID NO: 34 | 1054 aa | MW at 119613.5 kD |

| | |
|---|---|
| NOV6a, | MRQHDVQELNRILFSALETSLVGTSGHDLIYRLYHGTIVNQIVCKECKNVSERQEDFLDLTVAVKNVS |
| CG142182-01 | |
| Protein | GLEDALWNMYVEEEVFDCDNLYHCGTCDRLVKAAKSAKLRKLPPFLTVSLLRFNFDFVKCERYKETSC |
| Sequence | |
| | YTFPLRINLKPFCEQSELDDLEYIYDLFSVIIHKGGCYGGHYHVYIKDVDHLGNWQFQEEKSKPDVNL |
| | KDLQSEEEIDHPLMILKAILLEEENNLIPVDQLGQKLLKKIGISWNKKYRKQHGPLRKFLQLHSQIFL |
| | LSSDESTVRLLKNSSLQAESDFQRNDQQIFKMLPPESPGLNNSISCPHWFDINDSKVQPIREKDIEQQ |
| | FQGKESAYMLFYRKSQLQRPPEARANPRYGVPCHLLNEMDAANIELQTKRAECDSANNTFELHLHLGP |
| | QYHFFNGALHPVVSQTESVWDLTFDKRKTLGDLRQSIFQLLEFWEGDMVLSVAKLVPAGLHIYQSLGG |
| | DELTLCETEIADGEDIFVWNGVEVGGVHIQTGIDCEPLLLNVLHLDTSSDGEKCCQVIESPHVFPANA |
| | EVGTVLTALAIPAGVIFINSAGCPGGEGWTAIPKEDMRKTFREQGLRNGSSILIQDSHDDNSLLTKEE |
| | KWVTSMNEIDWLHVKNLCQLESEEKQVKISATVNTMVFDIRIKAIKELKLMKELADNSCLRPIDRNGK |
| | LLCPVPDSYTLKEAELKMGSSLGLCLGKAPSSSQLFLFFAMGSDVQPGTEMEIVVEETISVRDCLKLM |
| | LKKSGLQDSFIGDAWHLRKMDWCYEAGEPLCEEDATLKELLICSGDTLLLIEGQLPPLGFLKVPIWWY |
| | QLQGPSGHWESHQDQTNCTSSWGRVWRATSSQGASGNEPAQVSLLYLGDIEISEDATLAELKSQAMTL |
| | PPFLEFGVPSPAHLRAWTVERKRPGRLLRTDRQPLREYKLGRRIEICLEPLQKGENLGPQDVLLRTQV |
| | RIPGERTYAPALDLVWNAAQGGTAGSLRQRVADFYCLPVEKIEIAKYFPEKFEWLPISSWNQQITKRK |
| | KKKKQDYLQGAPYYLKDGDTIGVKVSCLTANLPL |

Further analysis of the NOV6a protein yielded the following properties shown in Table 6B.

TABLE 6B
Protein Sequence Properties NOV6a

| | |
|---|---|
| PSort analysis: | 0.7000 probability located in plasma membrane; |
| | 0.3500 probability located in nucleus; 0.3000 |
| | probability located in microbody (peroxisome); |
| | 0.2000 probability located in endoplasmic |
| | reticulum (membrane) |
| SignalP analysis: | No Known Signal Sequence Predicted |

A search of the NOV6a protein against the Geneseq database, a proprietary database that contains sequences published in patents and patent publication, yielded several homologous proteins shown in Table 6C.

TABLE 6C
Geneseq Results for NOV6a

| | | | Identities/ | |
|---|---|---|---|---|
| | | | Similarities | |
| Geneseq | Protein/Organism/Length | NOV6a Residues/ | for the | Expect |
| Identifier | [Patent #, Date] | Match Residues | Matched Region | Value |
| AAE14346 | Human protease PRTS-11 | 1 . . . 1044 | 1037/1044 (99%) | 0.0 |
| | protein - Homo sapiens, | 1 . . . 1040 | 1037/1044 (99%) | |
| | 1108 aa. | | | |
| | [WO200183775-A2, | | | |
| | 08 NOV. 2001] | | | |
| AAU68535 | Human novel cytokine | 1 . . . 1044 | 1037/1044 (99%) | 0.0 |
| | encoded by cDNA | 129 . . . 1167 | 1038/1044 (99%) | |
| | 790CIP2C_6 #1 - Homo | | | |
| | sapiens, 1346 aa. | | | |
| | [WO200175093-A1, | | | |
| | 11 OCT. 2001] | | | |
| AAB93169 | Human protein sequence | 1 . . . 1019 | 1013/1019 (99%) | 0.0 |
| | SEQ ID NO: 12102 - Homo | 1 . . . 1014 | 1013/1019 (99%) | |
| | sapiens, 1014 aa. | | | |
| | [EP1074617-A2, | | | |
| | 07 FEB. 2001] | | | |
| AAU68534 | Human novel cytokine | 1 . . . 1044 | 1015/1044 (97%) | 0.0 |
| | encoded by cDNA | 129 . . . 1145 | 1015/1044 (97%) | |
| | 790CIP2C_5 #1 - Homo | | | |
| | sapiens, 1324 aa. | | | |
| | [WO200175093-A1, | | | |
| | 11 OCT. 2001] | | | |
| ABG27066 | Novel human diagnostic | 500 . . . 666 | 166/168 (98%) | 4e−91 |
| | protein #27057 - Homo | 47 . . . 214 | 166/168 (98%) | |
| | sapiens, 674 aa. | | | |
| | [WO200175067-A2, | | | |
| | 11 OCT. 2001] | | | |

In a BLAST search of public sequence datbases, the NOV6a protein was found to have homology to the proteins shown in the BLASTP data in Table 6D.

TABLE 6D
Public BLASTP Results for NOV6a

| | | | Identities/ | |
|---|---|---|---|---|
| Protein | | | Similarities | |
| Accession | | NOV6a Residues/ | for the | Expect |
| Number | Protein/Organism/Length | Match Residues | Matched Portion | Value |
| Q9NVE5 | CDNA FLJ10785 fis, clone | 1 . . . 1019 | 1013/1019 (99%) | 0.0 |
| | NT2RP4000457, weakly | 1 . . . 1014 | 1013/1019 (99%) | |
| | similar to ubiquitin | | | |
| | carboxyl-terminal hydrolase | | | |
| | 15 (EC 3.1.2.15) - Homo | | | |
| | sapiens (Human), 1014 aa | | | |
| | (fragment). | | | |
| Q95KB6 | Hypothetical 102.2 kDa | 143 . . . 1024 | 844/882 (95%) | 0.0 |
| | protein - Macaca fascicularis | 30 . . . 907 | 860/882 (96%) | |
| | (Crab eating macaque) | | | |
| | (Cynomolgus monkey), 907 | | | |
| | aa (fragment). | | | |
| Q8S1J6 | Putative ubiquitin | 3 . . . 342 | 102/359 (28%) | 3e−23 |
| | carboxyl-terminal hydrolase - | 223 . . . 568 | 165/359 (45%) | |
| | Oryza sativa (japonica | | | |
| | cultivar-group), 1079 aa. | | | |
| Q8VZM4 | Putative ubiquitin | 3 . . . 202 | 72/205 (35%) | 3e−23 |
| | carboxyl-terminal hydrolase - | 278 . . . 480 | 105/205 (51%) | |
| | Arabidopsis thaliana | | | |
| | (Mouse-ear cress), 683 aa. | | | |
| Q94ED6 | Putative ubiquitin | 3 . . . 342 | 102/359 (28%) | 3e−23 |
| | carboxyl-terminal hydrolase - | 273 . . . 618 | 165/359 (45%) | |
| | Oryza sativa (Rice), 1108 | | | |
| | aa. | | | |

PFam analysis predicts that the NOV6a protein contains the domains shown in the Table 6E.

TABLE 6E
Domain Analysis of NOV6a

| | | Identities/ | |
|---|---|---|---|
| | | Similarities | |
| Pfam | NOV6a Match | for the | Expect |
| Domain | Region | Matched Region | Value |
| UCH-2 | 157 . . . 354 | 23/203 (11%) | 0.00033 |
| | | 141/203 (69%) | |

### Example 7

The NOV7 clone was analyzed, and the nucleotide and encoded polypeptide sequences are shown in Table 7A.

TABLE 7A
NOV7 Sequence Analysis

| | |
|---|---|
| SEQ ID NO: 35 | 692 bp |

| | |
|---|---|
| NOV7a, | GACAGGAGTGAACCCGAGCTGTGCCGACCAACCCCCAGGATGGCGGAAGCTCACCAGGCCGTGGCCTT |
| CG142564-01 | |
| DNA Sequence | CCAGTTCACGGTGACCCCAGACGGGGTCGACTTCCGGCTCAGTCGGGAGGCCCTGAAACACGTCTACC |
| | TGTCTGGGATCAACTCCTGGAAGAAACGCCTGATCCGCATCAAGAATGGCATCCTCAGGGGCGTGTAC |
| | CCTGGCAGCCCCACCAGCTGGCTGGTCGTCATCATGGTAACAGTGGGTTCCTCCTTCTGCAACGTGGA |
| | CATCTCCTTGGGGCTGGTCAGTTGCATCCAGAGATGCCTCCCTCAGGGGTGTGGCCCCTACCAGACCC |
| | CGCAGACCCGGGCACTTCTCAGCATGGCCATCTTCTCCACGGGCGTCTGGGTGACGGGCATCTTCTTC |
| | TTCCGCCAAACCCTGAAGCTGCTTCTCTGCTACCAATCCCAGATCCGCATGTTCGACCCAGAGCAGCA |
| | CCCCAATCACCTGGGCGCTGGAGGTGGCTTTGGCCCTGTAGCAGATGATGGCTATGGAGTTTCCTACA |
| | TGATTGCAGGCGAGAACACGATCTTCTTCCACATCTCCAGCAAGTTCTCAAGCTCAGAGACGAACGCC |
| | CAGCGCTTTGGAAACCACATCCGCAAAGCCCTGCTGGACATTGCTGATCTTTTCCAAGTTCCTCAGGC |
| | CTACAGCTGAAG |

| | | |
|---|---|---|
| ORF Start: ATG at 40 | | ORF Stop: TGA at 688 |
| SEQ ID NO: 36 | 216 aa | MW at 23874.3kD |

| | |
|---|---|
| NOV7a, | MAEAHQAVAFQFTVTPDGVDFRLSREALKHVYLSGINSWKKRLIRIKNGILRGVYPGSPTSWLVVIMV |
| CG142564-01 | |
| Protein | TVGSSFCNVDISLGLVSCIQRCLPQGCGPYQTPQTRALLSMAIFSTGVWVTGIFFFRQTLKLLLCYQS |
| Sequence | |
| | QIRMFDPEQHPNHLGAGGGFGPVADDGYGVSYMIAGENTIFFHISSKFSSSETNAQRFGNHIRKALLD |
| | IADLFQVPQAYS |

Further analysis of the NOV7a protein yielded the following properties shown in Table 7B.

TABLE 7B
Protein Sequence Properties NOV7a

| | |
|---|---|
| PSort analysis: | 0.7900 probability located in plasma membrane; |
| | 0.6400 probability located in microbody |
| | (peroxisome); 0.3000 probability located in |
| | Golgi body; 0.2000 probability located in |
| | endoplasmic reticulum (membrane) |
| SignalP analysis: | Cleavage site between residues 5 and 6 |

A search of the NOV7a protein against the Geneseq database, a proprietary database that contains sequences published in patents and patent publication, yielded several homologous proteins shown in Table 7C.

TABLE 7C
Geneseq Results for NOV7a

| | | NOV7a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAW14438 | Type I carnitine palmitoyl | 1 . . . 134 | 131/134 (97%) | 4e−72 |
| | transferase-like protein - | 1 . . . 134 | 131/134 (97%) | |
| | Homo sapiens, 772 aa. | | | |
| | [JP09009969-A, | | | |
| | 14 JAN. 1997] | | | |
| AAE10322 | Human carnitine | 1 . . . 134 | 57/134 (42%) | 1e−21 |
| | acyltransferase, 26886 - | 1 . . . 132 | 78/134 (57%) | |
| | Homo sapiens, 803 aa. | | | |
| | [WO200166759-A2, | | | |
| | 13 SEP. 2001] | | | |
| AAY79220 | Human transferase | 1 . . . 134 | 57/134 (42%) | 1e−21 |
| | TRNSFS-12 - Homo sapiens, | 1 . . . 132 | 78/134 (57%) | |
| | 803 aa. [WO200014251-A2, | | | |
| | 16 MAR. 2000] | | | |
| ABB67527 | Drosophila melanogaster | 137 . . . 210 | 43/74 (58%) | 6e−19 |
| | polypeptide SEQ ID NO | 688 . . . 761 | 55/74 (74%) | |
| | 29373 - Drosophila | | | |
| | melanogaster, 780 aa. | | | |
| | [WO200171042-A2, | | | |
| | 27 SEP. 2001] | | | |
| ABB66942 | Drosophila melanogaster | 137 . . . 210 | 43/74 (58%) | 6e−19 |
| | polypeptide SEQ ID NO | 690 . . . 763 | 55/74 (74%) | |
| | 27618 - Drosophila | | | |
| | melanogaster, 782 aa. | | | |
| | [WO200171042-A2, | | | |
| | 27 SEP. 2001] | | | |

In a BLAST search of public sequence datbases, the NOV7a protein was found to have homology to the proteins shown in the BLASTP data in Table 7D.

TABLE 7D
Public BLASTP Results for NOV7a

| | | | Identities/ | |
|---|---|---|---|---|
| Protein | | | Similarities for | |
| Accession | | NOV7a Residues/ | the Matched | Expect |
| Number | Protein/Organism/Length | Match Residues | Portion | Value |
| Q9BY90 | KIAA1670 protein - Homo | 1 . . . 134 | 133/134 (99%) | 2e−73 |
| | sapiens (Human), 598 aa | 18 . . . 151 | 133/134 (99%) | |
| | (fragment). | | | |
| Q92523 | Carnitine | 1 . . . 134 | 133/134 (99%) | 2e−73 |
| | O-palmitoyltransferase I, | 1 . . . 134 | 133/134 (99%) | |
| | mitochondrial muscle isoform | | | |
| | (EC 2.3.1.21) (CPT I) | | | |
| | (CPTI-M) (Carnitine | | | |
| | palmitoyltransferase I like | | | |
| | protein) - Homo sapiens | | | |
| | (Human), 772 aa. | | | |
| Q924X2 | Muscle-type carnitine | 1 . . . 149 | 118/149 (79%) | 1e−63 |
| | palmitoyltransferase I (EC | 1 . . . 147 | 128/149 (85%) | |
| | 2.3.1.21) (Hypothetical 88.2 | | | |
| | kDa protein) - Mus musculus | | | |
| | (Mouse), 772 aa. | | | |
| O35287 | Carnitine palmitoyltransferase | 1 . . . 149 | 118/149 (79%) | 1e−63 |
| | I - Mus musculus (Mouse), | 1 . . . 147 | 128/149 (85%) | |
| | 772 aa. | | | |
| Q9QYP4 | Muscle type carnitine | 1 . . . 149 | 118/149 (79%) | 1e−63 |
| | palmitoyltransferase I - Mus | 1 . . . 147 | 128/149 (85%) | |
| | musculus (Mouse), 772 aa. | | | |

### Example 8

The NOV8 clone was analyzed, and the nucleotide and encoded polypeptide sequences are shown in Table 8A.

TABLE 8A
NOV8 Sequence Analysis

| | |
|---|---|
| SEQ ID NO:37 | 1122 bp |

| | |
|---|---|
| NOV8a, | CTAGATTTTTGAAACATGAATCCTTCACTCCTCCTGGCTGCCTTTTTCCTGGGAATTGCCTCAGCTGC |
| CG142797-01 | |
| DNA Sequence | TCTAACACGTGACCACAGTCTAGACGCACAATGGACCAAGTGGAAGGCAAAGCACAAGAGATTATATG |
| | ACATGGAGAACATGAAGATGACTGAGCAGCACAATCAGGAATACAGCCAAGGGAAACACAGCTTCACA |
| | ATGGCCATGAACACCTTTGGAGACATGACCACTGAAGAATTCAGGCAGGTGATGAATGGTTTTCAATA |
| | CCAGAAGCACAGGAACGGGAAACAGTTCCAGGAACGCCTGCTTCTTGAGATCCCCACATCTGTGGACT |
| | GGAGAGAGAAAGGCTACATGACTCCTGTGAAGGATCAGGGTCAGTGTGGCTCTTGTTGGGCTTTTAGT |
| | GCAACTGGTGCTCTGGAAGGGCAGATGTTCTGGAAAACAGGCAAACTTATCTCACTGAATGAGCAGAA |
| | TCTGGTAGACTGCTCTGGGCCTCAAGGCAATGAGGGCTGCAATGGTGGCTTCATGGATAATCCCTTCC |
| | GGTATGTTCAGGAGAACGGAGGCCTGGACTCTGAGGCATCCTATCCATATGAAAAAACCTGTAGGTAC |
| | AATCCCAAGTATTCTGCTGCTAATGACACTGGCTTTGTGGACATCCCTTCACAGGAGAAGGACCTGGC |
| | GAAGGCAGTGGCAACTGTGGGGCCCATCTCTGTTGCTGCTGGTGCAAGCCATGTCTCCTTCCAGTTCT |
| | ATAAAAAAGGTATTTATTTTGAGCCACGCTGTGACCCCGAAGGTCTGGATCATGCTATGCTGCTGGTT |
| | GGCTACAGCTATGAAGGAGCAGACTCAGATAACAATAAATATTGGCTGGTGAAGAACAGGTATGGTAA |
| | AAACTGGGGCATGGATGGCTACATAAAGATGGCCAAAGACCGGAGGAACAACTGTGGAATTGCCACAG |
| | CAGCCAGCTACCCCACTGTGTGAGCTGATGGATGGTGATGAGGAAGAACTTGACTGAGGATGGCACAT |
| | CCAAAGGAGGAATTTATCTTCAATCTACCAGCCCCTGCTGTGTGGAATGCGCACTTCAATCATTGAAG |
| | ATCCAAGTGTGATTGGAATTCTGATATTTTCACA |

| | | |
|---|---|---|
| ORF Start: ATG at 16 | | ORF Stop: TGA at 973 |
| SEQ ID NO: 38 | 319 aa | MW at 35984.2 kD |

| | |
|---|---|
| NOV8a, | MNPSLLLAAFFLGIASAALTRDHSLDAQWTKWKAKHKRLYDMENMKMTEQHNQEYSQGKHSFTMAMNT |
| CG142797-01 | |
| Protein | FGDMTTEEFRQVMNGFQYQKHRNGKQFQERLLLEIPTSVDWREKGYMTPVKDGQGCGSCWAFSATGAL |
| Sequence | |
| | EGQMFWKTGKLISLNEQNLVDCSGPQGNEGCNGGFMDNPFRYVQENGGLDSEASYPYEKTCRYNPKYS |
| | AANDTGFVDIPSQEKDLAKAVATVGPISVAAGASHVSFQFYKKGIYFEPRCDPEGLDHAMLLVGYSYE |
| | GADSDNNKYWLVKNRYGKNWGMDGYIKMAKDRRNNCGIATAASYPTV |

Further analysis of the NOV8a protein yielded the following properties shown in Table 8B.

TABLE 8B
Protein Sequence Properties NOV8a

| | |
|---|---|
| PSort | 0.8200 probability located in endoplasmic reticulum |
| analysis: | (membrane); 0.5140 probability located in plasma |
| | membrane; 0.2423 probability located in |
| | microbody (peroxisome); 0.1000 probability located |
| | in endoplasmic reticulum (lumen) |
| SignalP | Cleavage site between residues 18 and 19 |
| analysis: | |

A search of the NOV8a protein against the Geneseq database, a proprietary database that contains sequences published in patents and patent publication, yielded several homologous proteins shown in Table 8C.

TABLE 8C
Geneseq Results for NOV8a

| | | | Identities/ | |
|---|---|---|---|---|
| | | | Similarities for | |
| Geneseq | Protein/Organism/Length | NOV8a Residues/ | the Matched | Expect |
| Identifier | [Patent #, Date] | Match Residues | Region | Value |
| AAU98883 | Human protease PRTS1 - | 1 . . . 319 | 303/334 (90%) | e−180 |
| | Homo sapiens, 334 aa. | 1 . . . 334 | 310/334 (92%) | |
| | [WO200238744-A2, | | | |
| | 16 MAY 2002] | | | |
| ABG61771 | Novel cathepsin-L | 1 . . . 319 | 288/333 (86%) | e−171 |
| | precursor-like protein - Homo | 1 . . . 333 | 300/333 (89%) | |
| | sapiens, 333 aa. | | | |
| | [WO200229058-A2, | | | |
| | 11 APR. 2002] | | | |
| ABG66692 | Human novel polypeptide | 1 . . . 319 | 260/333 (78%) | e−154 |
| | #27 - Homo sapiens, 333 aa. | 1 . . . 333 | 278/333 (83%) | |
| | [WO200244340-A2, | | | |
| | 06 JUN. 2002] | | | |
| ABG66714 | Human novel polypeptide | 1 . . . 319 | 259/333 (77%) | e−154 |
| | #49 - Homo sapiens, 333 aa. | 1 . . . 333 | 277/333 (82%) | |
| | [WO200244340-A2, | | | |
| | 06 JUN. 2002] | | | |
| ABB77396 | Human cathepsin L - Homo | 1 . . . 319 | 249/333 (74%) | e−147 |
| | sapiens, 333 aa. | 1 . . . 333 | 274/333 (81%) | |
| | [DE10050274-A1, | | | |
| | 18 APR. 2002] | | | |

In a BLAST search of public sequence datbases, the NOV8a protein was found to have homology to the proteins shown in the BLASTP data in Table 8D.

TABLE 8D
Public BLASTP Results for NOV8a

| | | | Identities/ | |
|---|---|---|---|---|
| Protein | | | Similarities for | |
| Accession | | NOV8a Residues/ | the Matched | Expect |
| Number | Protein/Organism/Length | Match Residues | Portion | Value |
| P07711 | Cathepsin L precursor (EC | 1 . . . 319 | 249/333 (74%) | e−147 |
| | 3.4.22.15) (Major excreted | 1 . . . 333 | 274/333 (81%) | |
| | protein) (MEP) - Homo | | | |
| | sapiens (Human), 333 aa. | | | |
| Q9GKL8 | Cysteine protease - | 1 . . . 319 | 247/333 (74%) | e−146 |
| | Cercopithecus aethiops (Green | 1 . . . 333 | 273/333 (81%) | |
| | monkey) (Grivet), 333 aa. | | | |
| Q9GL24 | Cathepsin L (EC 3.4.22.15) - | 1 . . . 319 | 236/334 (70%) | e−138 |
| | Canis familiaris (Dog), 333 aa. | 1 . . . 333 | 265/334 (78%) | |
| Q28944 | Cathepsin L precursor (EC | 1 . . . 319 | 228/334 (68%) | e−135 |
| | 3.4.22.15) - Sus scrofa (Pig), | 1 . . . 334 | 263/334 (78%) | |
| | 334 aa. | | | |
| P25975 | Cathepsin L precursor (EC | 1 . . . 319 | 222/334 (66%) | e−133 |
| | 3.4.22.15)- Bos taurus | 1 . . . 334 | 261/334 (77%) | |
| | (Bovine), 334 aa. | | | |

PFam analysis predicts that the NOV8a protein contains the domains shown in the Table 8E.

TABLE 8E
Domain Analysis of NOV8a

| | | Identities/ | |
|---|---|---|---|
| | NOV8a | Similarities | |
| | Match | for the Matched | Expect |
| Pfam Domain | Region | Region | Value |
| Peptidase_C1 | 103 . . . 318 | 123/337 (36%) | 2.4e−111 |
| | | 194/337 (58%) | |

### Example 9

The NOV9 clone was analyzed, and the nucleotide and encoded polypeptide sequences are shown in Table 9A.

TABLE 9A
NOV9 Sequence Analysis

| | |
|---|---|
| SEQ ID NO:32 | 1740 bp |

| | |
|---|---|
| NOV9a, | CACGAGGCCGCTAACGGTCCGGCGCCCCTCGGCGTCCGCGCGCCCCCAGCCTGGCGGACGAGCCCGGC |
| CG143216-01 | |
| DNA Sequence | GGCGGAGATGGGGGCGACGGGGGCGGCGGAGCCGCTGCAATCCGTGCTGTGGGTGAAGCAGCAGCGCT |
| | GCGCCGTGAGCCTGGAGCCCGCGCGGGCTCTGCTGCGCTGGTGGCGGAGCCCGGGGCCCGGAGCCGGC |
| | GCCCCCGGTGCTGATGCCTGCTCTGTGCCTGTATCTGAGATCATCGCCGTTGAGGAAACAGACGTTCA |
| | CGGGAAACATCAAGGCAGTGGAAAATGGCAGAAAATGGAAAAGCCTTACGCTTTTACAGTTCACTGTG |
| | TAAAGAGAGCACGACGGCACCGCTGGAAGTGGGCGCAGGTGACTTTCTGGTGTCCAGAGGAGCAGCTG |
| | TGTCACTTGTGGCTGCAGACCCTGCGGGAGATGCTGGAGAAGCTGACGTCCAGACCAAAGCATTTACT |
| | GGTATTTATCAACCCGTTTGGAGGAAAAGGACAAGGCAAGCGGATATATGAAAGAAAAGTGGCACCAC |
| | TGTTCACCTTAGCCTCCATCACCACTGACATCATCGTTACTGAACATGCTAATCAGGCCAAGGAGACT |
| | CTGTATGAGATTAACATAGACAAATACGACGGCATGTCTGTGTCGGCGGAGATCGGTATGTTCAGCGA |
| | GGTGCTGCACGGTCTGATTGGGAGGACGCAGAGGAGCGCCGGGGTCGACCAGAACCACCCCCGGGCTG |
| | TGCTGGTCCCCAGTAGCCTCCGGATTGGAATCATTCCCGCAGGGTCAACGGACTGCGTGTGTTACTCC |
| | ACCGTGGGCACCAGCGACGCAGAAACCTCGGCGCTGCATATCGTTGTTGGGGACTCGCTGGCCATGGA |
| | TGTGTCCTCAGTCCACCACCACAGCACACTCCTTCGCTACTCCGTGTCCCTGCTGGGCTACGGCTTCT |
| | ACGGGGACATCATCAAGGACAGTGAGAAGAAACGGTGGTTGGGTCTTGCCAGATACGACTTTTCAGGT |
| | TTAAAGACCTTCCTCTCCCACCACTGCTATGAAGGGACAGTGTCCTTCCTCCCTGCACAACACACGGT |
| | GGGATCTCCAAGGGATAGGAAGCCCTGCCGGGCAGGATGCTTTGTTTGCAGGCAAAGCAAGCAGCAGC |
| | TGGAGGAGGAGCAGAAGAAAGCACTGTATGGTTTGGAAGCTGCGGAGGACGTGGAGGAGTGGCAAGTC |
| | GTCTGTGGGAAGTTTCTGGCCATCAATGCCACAAACATGTCCTGTGCTTGTCGCCGGAGCCCCAGGGG |
| | CCTCTCCCCGGCTGCCCACTTGGGAGACGGGTCTTCTGACCTCATCCTCATCCGGAAATGCTCCAGGT |
| | TCAATTTTCTGAGATTTCTCATCAGGCACACCAACCAGCAGGACCAGTTTGACTTCACTTTTGTTGAA |
| | GTTTATCGCGTCAAGAAATTCCAGTTTACGTCGAAGCACATGGAGGATGAGGACAGCGACCTCAAGGA |
| | GGGGGGGAAGAAGCGCTTTGGGCACATTTGCAGCAGCCACCCCTCCTGCTGCTGCACCGTCTCCAACA |
| | GCTCCTGGAACTGCGACGGGGAGGTCCTGCACAGCCCTGCCATCGAGGTCAGAGTCCACTGCCAGCTG |
| | GTTCGACTCTTTGCACGAGGAATTGAAGAGAATCCGAAGCCAGACTCACACAGCTGAGAAGCCGGCGT |
| | CCTGCTCTCGAACTGGGAAAGTGTGAAAACTATTTAAGAT |

| | | |
|---|---|---|
| ORF Start: ATG at 76 | | ORF Stop: TGA at 1687 |
| SEQ ID NO: 40 | 537 aa | MW at 59976.9kD |

| | |
|---|---|
| NOV9a, | MGATGAAEPLQSVLWVKQQRCAVSLEPARALLRWWRSPGPGAGAPGADACSVPVSEIIAVEETDVHGK |
| CG143216-01 | |
| Protein | HQGSGKWQKMEKPYAFTVHCVKRARRHRWKWAQVTFWCPEEQLCHLWLQTLREMLEKLTSRPKHLLVF |
| Sequence | |
| | INPFGGKGQGKRIYERKVAPLFTLASITTDIIVTEHANQAKETLYEINIDKYDGIVCVGGDGMFSEVL |
| | HGLIGRTQRSAGVDQNHPRAVLVPSSLRIGIIPAGSTDCVCYSTVGTSDAETSALHIVVGDSLAMDVS |
| | SVHHNSTLLRYSVSLLGYGFYGDIIKDSEKKRWLGLARYDFSGLKTFLSHHCYEGTVSFLPAQHTVGS |
| | PRDRKPCRAGCFVCRQSKQQLEEEQKKALYGLEAAEDVEEWQVVCGKFLAINATNMSCACRRSPRGLS |
| | PAAHLGDGSSDLILIRKCSRFNFLRFLIRHTNQQDQFDFTFVEVYRVKKFQFTSKHMEDEDSDLKEGG |
| | KKRFGHICSSHPSCCCTVSNSSWNCDGEVLHSPAIEVRVHCQLVRLFARGIEENPKPDSHS |

Further analysis of the NOV9a protein yielded the following properties shown in Table 9B.

TABLE 9B
Protein Sequence Properties NOV9a

| | |
|---|---|
| PSort | 0.5121 probability located in microbody (peroxisome); |
| analysis: | 0.3000 probability located in nucleus; 0.1000 |
| | probability located in mitochondrial matrix space; |
| | 0.1000 probability located in lysosome (lumen) |
| SignalP | No Known Signal Sequence Predicted |
| analysis: | |

A search of the NOV9a protein against the Geneseq database, a proprietary database that contains sequences published in patents and patent publication, yielded several homologous proteins shown in Table 9C.

TABLE 9C
Geneseq Results for NOV9a

| | | | Identities/ | |
|---|---|---|---|---|
| Geneseq | Protein/Organism/Length | NOV9a Residues/ | Similarities for the | Expect |
| Identifier | [Patent #, Date] | Match Residues | Matched Region | Value |
| ABB07857 | Human sphingosine | 1 . . . 537 | 537/537 (100%) | 0.0 |
| | kinase-like protein - Homo | 26 . . . 562 | 537/537 (100%) | |
| | sapiens, 562 aa. | | | |
| | [WO200228906-A2, | | | |
| | 11 APR. 2002] | | | |
| ABB07856 | Human sphingosine | 1 . . . 537 | 537/537 (100%) | 0.0 |
| | kinase-like protein - Homo | 1 . . . 537 | 537/537 (100%) | |
| | sapiens, 537 aa. | | | |
| | [WO200228906-A2, | | | |
| | 11 APR. 2002] | | | |
| AAM49115 | Human ceramide kinase | 1 . . . 537 | 535/537 (99%) | 0.0 |
| | hCERK1 - Homo sapiens, | 1 . . . 537 | 536/537 (99%) | |
| | 537 aa. [WO200196575-A1, | | | |
| | 20 DEC. 2001] | | | |
| AAY96059 | Human sphingosine kinase | 78 . . . 537 | 458/460 (99%) | 0.0 |
| | C - Homo sapiens, 460 aa. | 1 . . . 460 | 459/460 (99%) | |
| | [WO200052173-A2, | | | |
| | 08 SEP. 2000] | | | |
| AAE07884 | Human sphingosine kinase | 78 . . . 537 | 459/471 (97%) | 0.0 |
| | (SphK) protein #2 - Homo | 1 . . . 471 | 460/471 (97%) | |
| | sapiens, 471 aa. | | | |
| | [WO200160990-A2, | | | |
| | 23 AUG. 2001] | | | |

In a BLAST search of public sequence datbases, the NOV9a protein was found to have homology to the proteins shown in the BLASTP data in Table 9D.

TABLE 9D
Public BLASTP Results for NOV9a

| Protein | | | Identities/ | |
|---|---|---|---|---|
| Accession | | NOV9a Residues/ | Similarities for the | Expect |
| Number | Protein/Organism/Length | Match Residues | Matched Portion | Value |
| Q8TCT0 | Putative lipid kinase - Homo | 1 . . . 537 | 537/537 (100%) | 0.0 |
| | sapiens (Human), 537 aa. | 1 . . . 537 | 537/537 (100%) | |
| Q9BYB3 | KIAA1646 protein - Homo | 57 . . . 537 | 481/481 (100%) | 0.0 |
| | sapiens (Human), 481 aa | 1 . . . 481 | 481/481 (100%) | |
| | (fragment). | | | |
| BAC01155 | Ceramide kinases - Mus | 1 . . . 529 | 450/529 (85%) | 0.0 |
| | musculus (Mouse), 531 aa. | 1 . . . 529 | 483/529 (91%) | |
| Q9UGE5 | DA59H18.2 (Novel protein | 130 . . . 444 | 314/326 (96%) | 0.0 |
| | similar to human, mouse, | 1 . . . 326 | 315/326 (96%) | |
| | yeast, worm and plant | | | |
| | (Predicted) proteins) - Homo | | | |
| | sapiens (Human), 326 aa | | | |
| | (fragment). | | | |
| Q9TZI1 | T10B11.2 protein - | 79 . . . 525 | 141/458 (30%) | 1e−52 |
| | Caenorhabditis elegans, 549 | 115 . . . 526 | 230/458 (49%) | |
| | aa. | | | |

PFam analysis predicts that the NOV9a protein contains the domains shown in the Table 9E.

TABLE 9E
Domain Analysis of NOV9a

| | | Identities/ | |
|---|---|---|---|
| | NOV9a | Similarities | |
| | Match | for the | Expect |
| Pfam Domain | Region | Matched Region | Value |
| PH | 32 . . . 124 | 9/93 (10%) | 0.38 |
| | | 64/93 (69%) | |
| DAGKc | 132 . . . 278 | 32/165 (19%) | 0.00015 |
| | | 100/165 (61%) | |

### Example 10

The NOV10 clone was analyzed, and the nucleotide and encoded polypeptide sequences are shown in Table 10A.

TABLE 10A
NOV10 Sequence Analysis

| | |
|---|---|
| SEQ ID NO:41 | 772 bp |

| | |
|---|---|
| NOV 10a, | AACTGGAGACCACAACTTCATGCTGCGTGGGATCTCCCAACTACCTGCAGTGGCCACCATGTCTTGG |
| CG143787-01 | |
| DNA Sequence | GTCCTGCTGCCTGTACTTTGGCTCATTGTTCAAACTCAAGCAATAGCCATAAAGCAAACACCTGAAT |
| | TAACGCTCCATGAAATAGTTTGTCCTAAAAAACTTCACATTTTACACAAAAGAGAGATCAAGAACAA |
| | CCAGACAGAAAAGCATGGCAAAGAGGAAAGGTATGAACCTGAAGTTCAATATCAGATGATCTTAAAT |
| | GGAGAAGAAATCATTCTCTCCCTACAAAAAACCAAGCACCTCCTGGGGCCAGACTACACTGAAACAT |
| | TGTACTCACCCAGAGGAGAGGAAATTACCACGAAACCTGAGAACATGGAACACTGTTACTATAAAGG |
| | AAACATCCTAAATGAAAAGAATTCTGTTGCCAGCATCAGTACTTGTGACGGGTTGAGAGGATACTTC |
| | ACACATCATCACCAAAGATACCTTTTATCTCAGAAACCAAAGTGCCTGCTGCAAGCACCTATTCCTA |
| | CAAATATAATGACAACACCAGTGTGTGGGAACCACCTTCTAGAAGTGGGAGAAGACTGTGATTGTGG |
| | CTCTCTTAAGGAGTGTACCAATCTCTGCTGTGAAGCCCTAACGTGTAAACTGAAGCCTGGAACTGAT |
| | TGCGGAGGAGATGCTCCAAACCATACCACAGAGTGAATCCAAAAGTCTGCTTCACTGAGATGCTACC |
| | TTGCCAGGACAAGAACCAAGAACTCTAACTGTCCC |

| | | |
|---|---|---|
| ORF Start: ATG at 20 | | ORF Stop: TGA at 704 |
| SEQ ID NO: 42 | 228aa | MW at 25718.4 kD |

| | |
|---|---|
| NOV10a, | MLRGISQLPAVATMSWVLLPVLWLIVQTQAIAIKQTPELTLHEIVCPKKLHILHKREIKNNQTEKHG |
| CG143787-01 | |
| Protein Sequence | KEERYEPEVQYQMILNGEEIILSLQKTKHLLGPDYTETLYSPRGEEITTKPENMEHCYYKGNILNEK |
| | NSVASISTCDGLRGYFTHHHQRYLLSQKPKCLLQAPIPTNIMTTPVCGNHLLEVGEDCDCGSLKECT |
| | NLCCEALTCKLKPGTDCGGDAPNHTTE |

| | |
|---|---|
| SEQ ID NO: 43 | 706 bp |

| | |
|---|---|
| NOV10b, | CACCGGATCCACCATGCTGCGTGGGATCTCCCAACTACCTGCAGTGGCCACCATGTCTTGGGTCCTG |
| 278889162 | |
| DNA Sequence | CTGCCTGTACTTTGGCTCATTGTTCAAACTCAAGCAATAGCCATAAAGCAAACACCTGAATTAACGC |
| | TCCATGAAATAGTTTGTCCTAAAAAACTTCACATTTTACACAAAAGAGAGATCAAGAACAACCAGAC |
| | AGAAAAGCATGGCAAAGAGGAAAGGTATGAACCTGAAGTTCAATATCAGATGATCTTAAATGGAGAA |
| | GAAATCATTCTCTCCCTACAAAAAACCAAGCACCTCCTGGGGCCAGACTACACTGAAACATTGTACT |
| | CACCCAGAGGAGAGGAAATTACCACGAAACCTGAGAACATGGAACACTGTTACTATAAAGGAAACAT |
| | CCTAAATGAAAAGAATTCTGTTGCCAGCATCAGTACTTGTGACGGGTTGAGAGGATACTTCACACAT |
| | CATCACCAAAGATACCTTTTATCTCAGAAACCAAAGTGCCTGCTGCAAGCACCTATTCCTACAAATA |
| | TAATGACAACACCAGTGTGTGGCAACCACCTTCTAGAAGTGGGAGAAGACTGTGATTGTGGCTCTCT |
| | TAAGGAGTGTACCAATCTCTGCTGTGAAGCCCTAACGTGTAAACTGAAGCCTGGAACTGATTGCGGA |
| | GGAGATGCTCCAAACCATACCACAGAGCTCGAGGGC |

| | | |
|---|---|---|
| ORF Start: at 2 | | ORF Stop: end of sequence |
| SEQ ID NO:45 | 118 bp | |

| | |
|---|---|
| NOV10c, | TGSTMLRGISQLPAVATMSWVLLPVLWLIVQTQAIAIKQTPELTLHEIVCPKKLHILHKREIKNNQT |
| 278689868 | |
| DNA Sequence | EKHGKEERYEPEVQYQMILNGEEIILSLQKTKHLLGPDYTETLYSPRGEEITTKPENMEHCYYKGNI |
| | LNEKNSVASISTCDGLRGYFTHHHQRYLLSQKPKCLLQAPIPTNIMTTPVCGNHLLEVGEDCDCGSL |
| | KECTNLCCEALTCKLKPGTDCGGDAPNHTTELEG |

| | | |
|---|---|---|
| ORF Start: at 2 | | ORF Stop: end of sequence |
| SEQ ID NO: 46 | 39 aa | MW at 3983.4 kD |

| | |
|---|---|
| NOV10c, | CACCGGATCCGAAGTGGGAGAAGACTGTGATTGTGGCTCTCTTAAGGAGTGTACCAATCTCTGCTGT |
| 278689868 | |
| DNA Sequence | GAAGCCCTAACGTGTAAACTGAAGCCTGGAACTGATTGCGGACTCGAGGGC |

| | | |
|---|---|---|
| ORF Start: at 2 | | ORF Stop: end of sequence |
| SEQ ID NO:46 | 39 aa | MW at 3983.4 kD |

| | |
|---|---|
| NOV10c, | TGSEVGEDCDCGSLKECTNLCCEALTCKLKPGTDCGLEG |
| 278689868 | |
| Protein Sequence | |

Sequence comparison of the above protein sequences yields the following sequence relationships shown in Table 10B.

TABLE 10B
Comparison of NOV10a against NOV10b and NOV10c.

| | | Identities/ |
|---|---|---|
| | | Similarities |
| | NOV10a Residues/ | for the |
| Protein Sequence | Match Residues | Matched Region |
| NOV10b | 1 . . . 228 | 228/228 (100%) |
| | 5 . . . 232 | 228/228 (100%) |
| NOV10c | 187 . . . 219 | 33/33 (100%) |
| | 4 . . . 36 | 33/33 (100%) |

Further analysis of the NOV10a protein yielded the following properties shown in Table 10C.

TABLE 10C
Protein Sequence Properties NOV10a

| | |
|---|---|
| PSort | 0.8200 probability located in outside; 0.1900 probability |
| analysis: | located in lysosome(lumen); 0.1000 probability located |
| | in endoplasmic reticulum (membrane);0.1000 probability |
| | located in endoplasmic reticulum (lumen) |
| SignalP | Cleavage site between residues 33 and 34 |
| analysis: | |

A search of the NOV10a protein against the Geneseq database, a proprietary database that contains sequences published in patents and patent publication, yielded several homologous proteins shown in Table 10D.

TABLE 10D
Geneseq Results for NOV10a

| | | | Identities/ | |
|---|---|---|---|---|
| | | | Similarities | |
| Geneseq | Protein/Organism/Length | NOV10a Residues/ | for the | Expect |
| Identifier | [Patent #, Date] | Match Residues | Matched Region | Value |
| AAW75769 | Human metalloproteinase | 1 . . . 157 | 157/157 (100%) | 7e−90 |
| | BS10.55 - Homo sapiens, | 1 . . . 157 | 157/157 (100%) | |
| | 470 aa. [WO9839421-A2, | | | |
| | 11 SEP. 1998] | | | |
| AAW28509 | Product of clone J5 - Homo | 1 . . . 157 | 157/157 (100%) | 7e−90 |
| | sapiens, 470 aa. | 1 . . . 157 | 157/157 (100%) | |
| | [WO9707198-A2, | | | |
| | 27 FEB. 1997] | | | |
| AAB53240 | Human colon cancer antigen | 153 . . . 228 | 73/76 (96%) | 7e−41 |
| | protein sequence SEQ ID | 35 . . . 110 | 74/76 (97%) | |
| | NO: 780 - Homo sapiens, 110 | | | |
| | aa. [WO200055351-A1, | | | |
| | 21 SEP. 2000] | | | |
| ABB11929 | Human eMDC II protein | 18 . . . 159 | 71/142 (50%) | 2e−32 |
| | homologue, SEQ ID | 18 . . . 153 | 99/142 (69%) | |
| | NO: 2299 - Homo sapiens, | | | |
| | 788 aa. [WO200157188-A2, | | | |
| | 09 AUG. 2001] | | | |
| AAW90865 | Human ADAM protein #4 - | 18 . . . 159 | 71/142 (50%) | 2e−32 |
| | Homo sapiens, 775 aa. | 5 . . . 140 | 99/142 (69%) | |
| | [WO200014227-A1, | | | |
| | 16 MAR. 2000] | | | |

In a BLAST search of public sequence datbases, the NOV10a protein was found to have homology to the proteins shown in the BLASTP data in Table 10E.

TABLE 10E
Public BLASTP Results for NOV10a

| | | | Identities/ | |
|---|---|---|---|---|
| Protein | | | Similarities | |
| Accession | | NOV10a Residues/ | for the | Expect |
| Number | Protein/Organism/Length | Match Residues | Matched Portion | Value |
| O15204 | Disintegrin-protease - Homo | 1 . . . 157 | 157/157 (100%) | 2e−89 |
| | sapiens (Human), 470 aa. | 1 . . . 157 | 157/157 (100%) | |
| Q9R0X2 | Disintegrin metalloprotease | 1 . . . 157 | 104/157 (66%) | 8e−56 |
| | precursor - Mus musculus | 1 . . . 157 | 124/157 (78%) | |
| | (Mouse), 467 aa. | | | |
| Q9XSL6 | ADAM 28 precursor (EC | 14 . . . 159 | 70/146 (47%) | 1e−32 |
| | 3.4.24.-) (A disintegrin and | 1 . . . 141 | 101/146 (68%) | |
| | metalloproteinase domain 28) | | | |
| | (eMDC II) - Macaca | | | |
| | fascicularis (Crab eating | | | |
| | macaque) (Cynomolgus | | | |
| | monkey), 776 aa. | | | |
| E1262181 | SEQUENCE 3 FROM | 18 . . . 159 | 71/142 (50%) | 5e−32 |
| | PATENT WO9709430 - | 5 . . . 140 | 99/142 (69%) | |
| | unidentified, 530 aa. | | | |
| Q9UKQ2 | ADAM 28 precursor (EC | 18 . . . 159 | 71/142 (50%) | 5e−32 |
| | 3.4.24.-) (A disintegrin and | 5 . . . 140 | 99/142 (69%) | |
| | metalloproteinase domain 28) | | | |
| | (Metalloproteinase-like, | | | |
| | disintegrin-like, and cysteine- | | | |
| | rich protein-L) (MDC-L) | | | |
| | (eMDC II) (ADAM23) - | | | |
| | Homo sapiens (Human), 775 | | | |
| | aa. | | | |

PFam analysis predicts that the NOV10 a protein contains the domains shown in the Table 10F.

TABLE 10F
Domain Analysis of NOV10a

| | | Identities/ | |
|---|---|---|---|
| | | Similarities | |
| Pfam | NOV10a Match | for the Matched | Expect |
| Domain | Region | Region | Value |
| Pep_M12B_propep | 90 . . . 201 | 32/119 (27%) | 1.8e−20 |
| | | 79/119 (66%) | |
| disintegrin | 187 . . . 219 | 20/33 (61%) | 4e−14 |
| | | 26/33 (79%) | |

### Example 11

The NOV11 clone was analyzed, and the nucleotide and encoded polypeptide sequences are shown in Table 11A.

TABLE 11A
NOV11 Sequence Analysis

| | |
|---|---|
| SEQ ID NO: 47 | 484 bp |

| | |
|---|---|
| NOV11a, | ACTGGGTCCGAATCAGTAGGTGACCCCGCCCCTGGATTCTGGAAGACCTCACCATGGGACGCCCCCG |
| CG144112-01 | |
| DNA Sequence | ACCTCGTGCGGCCAAGACGTGGATGTTCCTGCTCTTGCTGGGGGGAGCCTGGGCAGGAAATACACAG |
| | TACGCCTGGGAGACCACAGCCTACAGAATAAAGATGGCCCAGAAGTGCAGTCCCCGAGAGAATTTTC |
| | CTGACACTCTCAACTGTGCAGAAGTAAAAATCTTTCCCCAGAAGAAGTGTGAGGATGCTTACCCGGG |
| | GCAGATCACAGATGGCATGGTCTGTGCAGGCAGCAGCAAAGGGGCTGACACGTGCCAGGGCGATTCT |
| | GGAGGCCCCCTGGTGTGTGATGGTGCACTCCAGGGCATCACATCCTGGGGCTCAGACCCCTGTGGGA |
| | GGTCCGACAAACCTGGCGTCTATACCAACATCTGCCGCTACCTGGACTGGATCAAGAAGATCATAGG |
| | CAGCAAGGGCTGATT |

| | | |
|---|---|---|
| ORF Start: ATG at 54 | | ORF Stop: TGA at 480 |
| SEQ ID NO: 48 | 142 aa | MW at 15404.5 kD |

| | |
|---|---|
| NOV11a, | MGRPRPRAAKTWMFLLLLGGAWAGNTQYAWETTAYRIKMAQKCSPRENFPDTLNCAEVKIFPQKKCE |
| CG144112-01 | |
| Protein Sequence | DAYPGQITDGMVCAGSSKGADTCQGDSGGPLVCDGALQGITSWGSDPCGRSDKPGVYTNICRYLDWI |
| | KKIIGSKG |

| | |
|---|---|
| SEQ ID NO: 49 | 288 bp |

| | |
|---|---|
| NOV11b, | CCCCGCCCCTGGATTCTGGAAGACCTCACCATGGGACGCCCCCGACCTCGTGCGGCCAAGACGTGGA |
| CG144112-04 | |
| DNA Sequence | TGTTCCTGCTCTTGCTGGGGGGAGCCTGGGCAGGGCAGGGCGATTCTGGAGGCCCCCTGGTGTGTGA |
| | TGGTGCACTCCAGGGCATCACATCCTGGGGCTCAGACCCCTGTGGGAGGTCCGACAAACCTGGCGTC |
| | TATACCAACATCTGCCGCTACCTGGACTGGATCAAGAAGATCATAGGCAGCAAGGGCTGATTCTAGG |
| | ATAAGCACTAGATCTCCCTT |

| | | |
|---|---|---|
| ORF Start: ATG at 31 | | ORF Stop: TGA at 259 |
| SEQ ID NO: 50 | 76 aa | MW at 8110.3 kD |

| | |
|---|---|
| NOV11b, | MGRPRPRAAKTWMFLLLLGGAWAGQGDSGGPLVCDGALQGITSWGSDPCGRSDKPGVYTNICRYLDW |
| CG144112-04 | |
| Protein Sequence | IKKIIGSKG |

| | |
|---|---|
| SEQ ID NO: 51 | 445 bp |

| | |
|---|---|
| NOV11c, | CACCAAGCTTATGGGACGCCCCCGACCTCGTGCGGCCAAGACGTGGATGTTCCTGCTCTTGCTGGGG |
| 255501898 | |
| DNA Sequence | GGAGCCTGGGCAGGAAATACACAGTACGCCTGGGAGACCACAGCCTACAGAATAAAGATGGCCCAGA |
| | AGTGCAGTCCCCGAGAGAATTTTCCTGACACTCTCAACTGTGCAGAAGTAAAAATCTTTCCCCAGAA |
| | GAAGTGTGAGGATGCTTACCCGGGGCAGATCACAGATGGCATGGTCTGTGCAGGCAGCAGCAAAGGG |
| | GCTGACACGTGCCAGGGCGATTCTGGAGGCCCCCTGGTGTGTGATGGTGCACTCCAGGGCATCACAT |
| | CCTGGGGCTCAGACCCCTGTGGGAGGTCCGACAAACCTGGCGTCTATACCAACATCTGCCGCTACCT |
| | GGACTGGATCAAGAAGATCATAGGACAGCAAGGGCCTCGAGGGC |

| | | |
|---|---|---|
| ORF Start: at 2 | | ORF Stop: end of sequence |
| SEQ ID NO:52 | 148 aa | MW at 16046.2 kD |

| | |
|---|---|
| NOV11c | TKLMGRPRPRAAKTWMFLLLLGGAWAGNTQYAWETTAYRIKMAQKCSPRENFPDTLNCAEVKIFPQK |
| 255501898 | |
| Protein Sequence | KCEDAYPGQITDGMVCAGSSKGADTCQGDSGGPLVCDGALQGITSWGSDPCGRSDKPGVYTNICRYL |
| | DWIKKIGSKGLEG |

| | |
|---|---|
| SEQ ID NO:53 | 358 bp |

| | |
|---|---|
| NOV11d, | CACCAAGCTTGGAAATACACAGTACGCCTGGGAGACCACAGCCTACAGAATAAAGATGGCCCAGAAG |
| 255612524 | |
| DNA Sequence | TGCAGTCCCCGAGAGAATTTTCCTGACACTCTCAACTGTGCAGAAGTAAAAATCTTTCCCCAGAAGA |
| | AGTGTGAGGATGCTTACCCGGGGCAGATCACAGATGGCATGGTCTGTGCAGGCAGCAGCAAAGGGGC |
| | TGACACGTGCCAGGGCGATTCTGGAGGCCCCCTGGTGTGTGATGGTGCACTCCAGGGCATCACATCC |
| | TGGGGCTCAGACCCCTGTGGGAGGTCCGACAAACCTGGCGTCTATACCAACATCTGCCGCTACCTGG |
| | ACTGGATCAAGAAGCTCGAGGGC |

| | | |
|---|---|---|
| ORF Start: at 2 | | ORF Stop: end of sequence |
| SEQ ID NO: 54 | 119 aa | MW at 12908.4 kD |

| | |
|---|---|
| NOV11d, | TKLGNTQYAWETTAYRIKMAQKCSPRENFPDTLNCAEVKIFPQKKCEDAYPGQITDGMVCAGSSKGA |
| 255612524 | |
| Protein Sequence | DTCQGDSGGPLVCDGALQGITSWGSDPCGRSDKPGVYTNICRYLDWIKKLEG |

| | |
|---|---|
| SEQ ID NO: 55 | 307 bp |

| | |
|---|---|
| NOV11e, | CACCAAGCTTCAGAAGTGCAGTCCCCGAGAGAATTTTCCTGACACTCTCAACTGTGCAGAAGTAAAA |
| 255612566 | |
| DNA Sequence | ATCTTTCCCCAGAAGAAGTGTGAGGATGCTTACCCGGGGCAGATCACAGATGGCATGGTCTGTGCAG |
| | GCAGCAGCAAAGGGGCTGACACGTGCCAGGGCGATTCTGGAGGCCCCCTGGTGTGTGATGGTGCACT |
| | CCAGGGCATCACATCCTGGGGCTCAGACCCCTGTGGGAGGTCCGACAAACCTGGCGTCTATACCAAC |
| | ATCTGCCGCTACCTGGACTGGATCAAGAAGCTCGAGGGC |

| | | |
|---|---|---|
| ORF Start: at 2 | | ORF Stop: end of sequence |
| SEQ ID NO: 56 | 102 aa | MW at 10922.2 kD |

| | |
|---|---|
| NOV11e, | TKLQKCSPRENFPDTLNCAEVKIFPQKKCEDAYPGQITDGMVCATSSKGADTCQGDSGGPLVCDGAL |
| 256612566 | |
| Protein Sequence | QGITSWGSDPCGRSDKPGVYTNICRYLDWIKKLEG |

| | |
|---|---|
| SEQ ID NO: 57 | 178 bp |

| | |
|---|---|
| NOV11f, | CACCGGATCCGGGCAGGGCGATTCTGGAGGCCCCCTGGTGTGTGATGGTGCACTCCAGGGCATCACA |
| 306434072 | |
| DNA Sequence | TCCTGGGGCTCAGACCCCTGTGGGAGGTCCGACAAACCTGGCGTCTATACCAACATCTGCCGCTACC |
| | TGGACTGGATCAAGAAGATCATAGGCAGCAAGGGCCTCGAGGGC |

| | | |
|---|---|---|
| ORF Start: at 2 | | ORF Stop: end of sequence |
| SEQ ID NO: 58 | 59 aa | MW at 6072.7 kD |

| | |
|---|---|
| NOV11f, | TGSGQGDSGGPLVCDGALQGITSWGSDPCGRSDKPGVYTNICRYLDWIKKIIGSKGLEG |
| 306434072 | |
| Protein Sequence | |

| | |
|---|---|
| SEQ ID NO: 59 | 436 bp |

| | |
|---|---|
| NOV11g, | AGTGTGCTGGAATTCGCCCTTACTGGGTCCGAATCAGTAGGTGACCCCGCCCCTGGATTCTTGAAGA |
| CG144112-02 | |
| DNA Sequence | CCTCACCATGGGACGCCCCCGACCTCGTGCGGCCAAGACGTGGATGTTCCTGCTCTTGCTGGGGGGA |
| | GCCTGGGCAGAGAATTTTCCTGACACTCTCAACTGTGCAGAAGTAAAAATCTTTCCCCAGAAGAAGT |
| | GTGAGGATGCTTACCCGGGGCAGATCACAGATGGCATGGTCTGTGCAGGCAGCAGCAAAGGGGCTGA |
| | CACGTGCCAGGGCGATTCTGGAGGCCCCCTGGTGTGTGATGGTGCACTCCAGGGCATCACATCCTGG |
| | GGCTCAGACCCCTGTGGGAGGTCCGACAAACCTGGCGTCTATACCAACATCTGCCGCTACCTGGACT |
| | GGATCAAGAAGATCATAGGCAGCAAGGGCTGATT |

| | | |
|---|---|---|
| ORF Start: ATG at 75 | | ORF Stop: TGA at 432 |
| SEQ ID NO: 60 | 119 aa | MW at 12718.4kD |

| | |
|---|---|
| NOV11g, | MGRPRPRAAKTWMFLLLLGGAWAENFPDTLNCAEVKIFPQKKCEDAYPGQITDGMVCAGSSKGADTC |
| CG144112-02 | |
| Protein Sequence | QCDSGGPLVCDGALQGITSWGSDPCGRSDKPGVYTNICRYLDWIKKIIGSKG |

| | |
|---|---|
| SEQ ID NO: 61 | 845 bp |

| | |
|---|---|
| NOV11h, | CGCCCTTACTGGGTCCGAATCAGTAGGTGACCCCGCCCCTGGATTCTGGAAGACCTCACCATGGGAC |
| CG144112-03 | |
| DNA Sequence | GCCCCCGACCTCGTGCGGCCAAGACGTGGATGTTCCTGCTCTTGCTGGGGGGAGCCTGGGCAGGACA |
| | CTCCAGGGCACAGGAGGACAAGGTGCTGGGGGGTCATGAGTGCCAACCCCATTCGCAGCCTTGGCAG |
| | GCGGCCTTGTTCCAGGGCCAGCAACTACTCTGTGGCGGTGTCCTTGTAGGTGGCAACTGGGTCCTTA |
| | CAGCTGCCCACTGTAAAAAACCGAAATACACAGTACGCCTGGGAGACCACAGCCTACAGAATAAAGA |
| | TGGCCCAGAGCAAGAAATACCTGTGGTTCAGTCCATCCCACACCCCTGCTACAACAGCAGCGATGTG |
| | GAGGACCACAACCATGATCTGATGCTTCTTCAACTGCGTGACCAGGCATCCCTGGGGTCCAAAGTGA |
| | AGCCCATCAGCCTGGCAGATCATTGCACCCAGCCTGGCCAGAAGTGCACCGTCTCAGGCTGGGGCAC |
| | TGTCACCAGTCCCCGAGAGAATTTTCCTGACACTCTCAACTGTGCAGAAGTAAAAATCTTTCCCCAG |
| | AAGAAGTGTGAGGATGCTTACCCGGGGCAGATCACAGATGGCATGGTCTGTGCAGGCAGCAGCAAAG |
| | GGGCTGACACGTGCCAGGGCGATTCTGGAGGCCCCCTGGTGTGTGATGGTGCACTCCAGGGCATCAC |
| | ATCCTGGGGCTCAGACCCCTGTGGGAGGTCCGACAAACCTGGCGTCTATACCAACATCTGCCGCTAC |
| | CTGGACTGGATCAAGAAGATCATAGGCAGCAAGGGCTGATT |

| | | |
|---|---|---|
| ORF Start: ATG at 61 | | ORF Stop: TGA at 841 |
| SEQ ID NO: 62 | 260 aa | MW at 28047.6 kD |

| | |
|---|---|
| NOV11h, | MGRPRPRAAKTWMFLLLLGGAWAGHSRAQEDKVLGGHECQPHSQPWQAALFQGQQLLCGGVLVGGNW |
| CG114112-03 | |
| Protein Sequence | VLTAAHCKKPKYTVRLGDHSLQNKDGPEQEIPVVQSIPHPCYNSSDVEDHNHDLMLLQLRDQASLGS |
| | KVKPISLADHCTQPGQKCTVSGWGTVTSPRENFPDTLNCAEVKIFPQKKCEDAYPGQITDGMVCAGS |
| | SKGADTCQGDSGGPLVCDGALQGITSWGSDPCGRSDKPGVYTNICRYLDWIKKIIGSKG |

Sequence comparison of the above protein sequences yields the following sequence relationships shown in Table 11B.

TABLE 11B
Comparison of NOV11a against NOV11b through NOV11h.

| | | Identities/ |
|---|---|---|
| | NOV11a Residues/ | Similarities for |
| Protein Sequence | Match Residues | the Matched Region |
| NOV11b | 97 . . . 142 | 46/46 (100%) |
| | 31 . . . 76 | 46/46 (100%) |
| NOV11c | 1 . . . 142 | 142/142 (100%) |
| | 4 . . . 145 | 142/142 (100%) |
| NOV11d | 24 . . . 139 | 114/116 (98%) |
| | 4 . . . 119 | 115/116 (98%) |
| NOV11e | 41 . . . 139 | 97/99 (97%) |
| | 4 . . . 102 | 98/99 (98%) |
| NOV11f | 91 . . . 142 | 52/52 (100%) |
| | 5 . . . 56 | 52/52 (100%) |
| NOV11g | 1 . . . 142 | 119/142 (83%) |
| | 1 . . . 119 | 119/142 (83%) |
| NOV11h | 44 . . . 142 | 99/99 (100%) |
| | 162 . . . 260 | 99/99 (100%) |

Further analysis of the NOV11 a protein yielded the following properties shown in Table 11C.

TABLE 11C
Protein Sequence Properties NOV11a

| | |
|---|---|
| PSort analysis: | 0.3700 probability located in outside; 0.1000 |
| | probability located in endoplasmic reticulum |
| | (membrane); 0.1000 probability located in |
| | endoplasmic reticulum (lumen); 0.1000 |
| | probability located in lysosome (lumen) |
| SignaIP analysis: | Cleavage site between residues 24 and 25 |

A search of the NOV11a protein against the Geneseq database, a proprietary database that contains sequences published in patents and patent publication, yielded several homologous proteins shown in Table 11D.

TABLE 11D
Geneseq Results for NOV11a

| | | NOV11a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABP41332 | Human ovarian antigen | 44 . . . 142 | 99/99 (100%) | 3e−57 |
| | HCOQP78, SEQ ID NO: 2464 - | 217 . . . 315 | 99/99 (100%) | |
| | Homo sapiens, 315 aa. | | | |
| | [WO200200677-A1, | | | |
| | 03-JAN-2002] | | | |
| AAU81959 | Human PR0322 - Homo | 44 . . . 142 | 99/99 (100%) | 3e−57 |
| | sapiens, 260 aa. | 162 . . . 260 | 99/99 (100%) | |
| | [WO200109327-A2, | | | |
| | 08-FEB-2001] | | | |
| ABB84852 | Human PR0322 protein | 44 . . . 142 | 99/99 (100%) | 3e−57 |
| | sequence SEQ ID NO: 72 - | 162 . . . 260 | 99/99 (100%) | |
| | Homo sapiens, 260 aa. | | | |
| | [WO200200690-A2, | | | |
| | 03-JAN-2002] | | | |
| ABB95458 | Human angiogenesis related | 44 . . . 142 | 99/99 (100%) | 3e−57 |
| | protein PR0322 SEQ ID NO: | 162 . . . 260 | 99/99 (100%) | |
| | 72 - Homo sapiens, 260 aa. | | | |
| | [WO200208284-A2, | | | |
| | 31-JAN-2002] | | | |
| AAB53087 | Human | 44 . . . 142 | 99/99 (100%) | 3e−57 |
| | angiogenesis-associated | 162 . . . 260 | 99/99 (100%) | |
| | protein PR0322, SEQ ID | | | |
| | NO: 127 - Homo sapiens, 260 | | | |
| | aa. [WO200053753-A2, | | | |
| | 14-SEP-2000] | | | |

In a BLAST search of public sequence datbases, the NOV11a protein was found to have homology to the proteins shown in the BLASTP data in Table 11E.

TABLE 11E
Public BLASTP Results for NOV11a

| | | NOV11a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9NR68 | Serine protease | 1 . . . 142 | 119/142 (83%) | 9e−66 |
| | kallikrein/ovasin/neuropsin | 1 . . . 119 | 119/142 (83%) | |
| | Type 3 - Homo sapiens | | | |
| | (Human), 119 aa. | | | |
| 060259 | Neuropsin precursor (EC | 44 . . . 142 | 99/99 (100%) | 9e−57 |
| | 3.4.21.-) (NP) (Kallikrein 8) | 162 . . . 260 | 99/99 (100%) | |
| | (Ovasin) (Serine protease | | | |
| | TADG-14) | | | |
| | (Tumor-associated | | | |
| | differentially expressed | | | |
| | gene-14 protein) - Homo | | | |
| | sapiens (Human), 260 aa. | | | |
| 088780 | Neuropsin precursor (EC | 38 . . . 141 | 80/113 (70%) | 8e−45 |
| | 3.4.21.-) (NP) (Kallikrein 8) | 147 . . . 259 | 93/113 (81%) | |
| | (Brain serine protease 1) - | | | |
| | Rattus norvegicus (Rat), 260 | | | |
| | aa. | | | |
| BAB92021 | Neuropsin-Musmusculus | 38 . . . 141 | 81/113 (71%) | le−44 |
| | (Mouse), 176 aa (fragment). | 63 . . . 175 | 92/113 (80%) | |
| Q61955 | Neuropsin precursor (EC | 38 . . . 141 | 81/113 (71%) | le−44 |
| | 3.4.21.-) (NP) (Kallikrein 8)- | 147 . . . 259 | 92/113 (80%) | |
| | Mus musculus (Mouse), 260 | | | |
| | aa. | | | |

PFam analysis predicts that the NOV11a protein contains the domains shown in the Table 11F.

TABLE 11F
Domain Analysis of NOV11a

| | | Identities/ | |
|---|---|---|---|
| Pfam | NOV11a | Similarities | Expect |
| Domain | Match Region | for the Matched Region | Value |
| trypsin | 49 . . . 134 | 47/101 (47%) | 5.5e−40 |
| | | 76/101 (75%) | |

### Example 12

The NOV12 clone was analyzed, and the nucleotide and encoded polypeptide sequences are shown in Table 12A.

TABLE 12A
NOV12 Sequence Analysis

| | |
|---|---|
| SEQ ID NO:63 | 1536 bp |

| | |
|---|---|
| NOV12a, | AAGAGCCAAGCCAGCATGTCGGGGACCCGAGCCTCCAACGACCGGCCCCCCGGCGCAGGCGGCGTCA |
| CG14497-01 | |
| DNA Sequence | AGCGGGGGCGGCTGCAGCAGGAGGCGGCGGCGACCGGCTCCCGCGTGACGGTGGTGCTGGGCGCGCA |
| | GTGGGGGGACGAGGGCAAAGGCAAGGTGGTGGACCTGCTGGCCACGGACGCCGACATCATCAGCCGC |
| | TGCCAGGGGGGCAACAACGCCGGCCACACGGTGGTGGTGGATGGGAAAGAGTACGACTTCCACCTGC |
| | TGCCCAGCGGCATCATCAACACCAAGGCCGTGTCCTTCATTGGTAACGGGGTGGTCATCCACTTGCC |
| | AGGCTTGTTTGAGGAAGCAGAGAAGAATGAAAAGAAACGTCTGAAGGACTGGGAGAAGAGGCTCATC |
| | ATCTCTGACAGAGCCCACCTTGTGTTTGATTTTCACCAGGCTGTCGACGGACTTCAGGAAGTGCAGC |
| | GCCAGGCACAAGAGGGGAAGAGTATAGGCACCACCAAGAAGGGAATCGGACCAACCTACTCTTCCAA |
| | AGCTGCCCGGACAGGCCTCCGCATCTGCGACCTCCTGTCAGATTTTGATGAGTTTTCCTCCAGATTC |
| | AAGAACCTGGCCCACCAGCACCAGTCGATGTTCCCCACCCTGGAAATAGACATTGAAGGCCAACTCA |
| | AAAGGCTCAAGGGCTTTGCTGAGCGGATCAGACCCATGGTCCGAGATGGTGTTTACTTTATGTATGA |
| | GGCACTCCACGGCCCCCCCAAGAAGATCCTGGTGGAGGGTGCCAACGCCGCCCTCCTCGACATTGAC |
| | TTCGGTACCTACCCCTTTGTGACTTCATCCAACTGCACCGTGGGCGGTGTGTGCACGGGCCTGGGCA |
| | TCCCCCCGCAGAACATAGGTGACGTGTATGGCGTGGTGAAAGCCTATACCACACGTGTGGGCATCGG |
| | GGCCTTCCCCACCGAGCAGATCAACGAGATTGGAGGCCTGCTGCAGACCCGCGGCCACGAGTGGGGA |
| | GTGACCACAGGCAGGAAGAGGCGCTGCGGCTGGCTCGACCTGATGATTCTAAGATATGCTCACATGG |
| | TCAACGGATTCACTGCGCTGGCCCTGACGAAGCTGGACATCCTGGACGTACTGGGTGAGGTTAAAGT |
| | CGGTGTCTCATACAAGCTGAACGGGAAAAGGATTCCCTATTTCCCAGCTAACCAGGAGATGCTTCAG |
| | AAGGTCGAAGTTGAGTATGAAACGCTGCCTGGGTGGAAAGCAGACACCACAGGCGCCAGGAGGTGGG |
| | AGGACCTGCCCCCACAGGCCCAGAACTACATCCGCTTTGTGGAGAATCACGTGGGAGTCGCAGTCAA |
| | ATGGGTTGGTGTTGGCAAGTCAAGAGAGTCGATGATCCAGCTGTTTTAGTCACAGACTGAGCTGATC |
| | CCAACAGGCCCTGGCAGCGTCTGGACTTGTGTAAACAGCAGCAGTCACGTTCCTCGGCCGCCACAAC |
| | CAACACCAAAGCAGGAAAACCATTTTCTGTACTTTTATATTTCTGTTCAACCTGTTGGTTTC |

| | | |
|---|---|---|
| ORF Start: ATG at 16 | | ORF Stop: TAG at 1387 |
| SEQ ID NO: 64 | 457aa | MW at 50181.0 kD |

| | |
|---|---|
| NOV12a, | MSGTRASNDRPPGAGGVKRGRLQQEAAATGSRVTVVLGAQWGDEGKGKVVDLLATDATIISRCQGGN |
| CG144497-01 | |
| Protein Sequence | NAGHTVVVDGKEYDFHLLPSGIINTKAVSFIGNGVVIHLPGLFEEAEKNEKKGLKDWEKRLIISDRA |
| | HLVFDFHQAVDGLQEVQRQAQEGKSIGTTKKGIGPTYSSKAARTGLRICDLLSDFDEFSSRFKNLAH |
| | QHQSMFPTLEIDIEGQLKRLKGFAERIRPMVRDGVYFMYEALHGPPKKILVEGANAALLDIDFGTYP |
| | FVTSSNCTVGGVCTGLGIPPQNIGDVYGVVKAYTTRVGIGAFPTEQINEIGGLLQTRGHEWGVTTGR |
| | KRRCGWLDLMILRYAHMVNGFTALALTKLDILDVLGEVKVGVSYKLNGKRIPYFPANQEMLQKVEVE |
| | YETLPGWKADTTGARRWEDLPPQAQNYIRFVENHVGVAVKWVGVGKSRESMIQLF |

Further analysis of the NOV12a protein yielded the following properties shown in Table 12B.

TABLE 12B
Protein Sequence Properties NOV12a

| | |
|---|---|
| PSort analysis: | 0.5946 probability located in microbody |
| | (peroxisome); 0.3000 probability located in nucleus; |
| | 0.2377 probability located in lysosome (lumen); |
| | 0.1000 probability located in mitochondrial |
| | matrix space |
| SignalP analysis: | No Known Signal Sequence Predicted |

A search of the NOV 12a protein against the Geneseq database, a proprietary database that contains sequences published in patents and patent publication, yielded several homologous proteins shown in Table 12C.

TABLE 12C
Geneseq Results for NOV12a

| | | NOV12a | | |
|---|---|---|---|---|
| | | Residues/ | Identities/Similarities | |
| Geneseq | Protein/Organism/Length | Match | for the Matched | Expect |
| Identifier | [Patent #, Date] | Residues | Region | Value |
| AAB41627 | Human ORFX ORF1391 | 144 . . . 457 | 313/314 (99%) | 0.0 |
| | polypeptide sequence SEQ ID NO: | 1 . . . 314 | 314/314 (99%) | |
| | 2782 - Homo sapiens, | | | |
| | 314 aa. [WO200058473-A2, | | | |
| | 05-OCT-2000] | | | |
| ABB70971 | Drosophila melanogaster | 31 . . . 456 | 270/427 (63%) | e−161 |
| | polypeptide SEQ ID NO | 24 . . . 446 | 338/427 (78%) | |
| | 39705 - Drosophila | | | |
| | melanogaster, 447 aa. | | | |
| | [WO200171042-A2, | | | |
| | 27-SEP-2001] | | | |
| AAY95049 | Candida albicans polypeptide | 35 . . . 455 | 227/425 (53%) | e−130 |
| | sequence #17 - Candida | 4 . . . 409 | 306/425 (71%) | |
| | albicans, 412 aa. | | | |
| | [EP982401-A2, | | | |
| | 01-MAR-2000] | | | |
| AAU23499 | Novel human enzyme | 249 . . . 457 | 208/209 (99%) | e−121 |
| | polypeptide #585 - Homo | 1 . . . 209 | 209/209 (99%) | |
| | sapiens, 209 aa. | | | |
| | [WO200155301-A2, | | | |
| | 02-AUG-2001] | | | |
| AAW99455 | Maize adenylosuccinate | 24 . . . 454 | 217/436 (49%) | e−119 |
| | synthetase - Zea mays, 484 | 53 . . . 482 | 310/436 (70%) | |
| | aa. [US5882869-A, | | | |
| | 16-MAR-1999] | | | |

In a BLAST search of public sequence datbases, the NOV12a protein was found to have homology to the proteins shown in the BLASTP data in Table 12D.

Table 12D
Public BLASTP Results for NOV12a

| | | NOV12a | Identities/ | |
|---|---|---|---|---|
| Protein | | Residues/ | Similarities for | |
| Accession | | Match | the Matched | Expect |
| Number | Protein/Organism/Length | Residues | Portion | Value |
| BAC04649 | CDNA FLJ38602 fis, clone | 1 . . . 457 | 456/457 (99%) | 0.0 |
| | HEART2003836, highly | 1 . . . 457 | 457/457 (99%) | |
| | similar to | | | |
| | ADENYLOSUCCINATE | | | |
| | SYNTHETASE, MUSCLE | | | |
| | ISOZYME (EC 6.3.4.4) - | | | |
| | Homo sapiens (Human), 457 | | | |
| | aa. | | | |
| P28650 | Adenylosuccinate synthetase, | 1 . . . 457 | 441/457 (96%) | 0.0 |
| | muscle isozyme (EC 6.3.4.4) | 1 . . . 457 | 453/457 (98%) | |
| | (IMP-- aspartate ligase) | | | |
| | (ADSS) (AMPSASE) - Mus | | | |
| | musculus (Mouse), 457 aa. | | | |
| AJMSDS | adenylosuccinate synthase | 1 . . . 425 | 411/425 (96%) | 0.0 |
| | (EC 6.3.4.4), muscle - mouse, | 1 . . . 425 | 421/425 (98%) | |
| | 452 aa. | | | |
| AAH32039 | Similar to | 64 . . . 457 | 392/394 (99%) | 0.0 |
| | ADENYLOSUCCINATE | 109 . . . 502 | 394/394 (99%) | |
| | SYNTHETASE, MUSCLE | | | |
| | ISOZYME | | | |
| | (IMP--ASPARTATE | | | |
| | LIGASE) (ADSS) | | | |
| | (AMIPSASE) - Homo sapiens | | | |
| | (Human), 502 aa (fragment). | | | |
| Q9CQL9 | Adenylosuccinate synthetase | 8 . . . 457 | 345/453 (76%) | 0.0 |
| | (EC 6.3.4.4) (IMP--aspartate | 4 . . . 456 | 399/453 (87%) | |
| | ligase) (ADSS) (AMPSase) - | | | |
| | Mus musculus (Mouse), 456 | | | |
| | aa. | | | |

PFam analysis predicts that the NOV12a protein contains the domains shown in the Table 12E.

TABLE 12E
Domain Analysis of NOV12a

| | | Identities/ | |
|---|---|---|---|
| Pfam | NOV12a Match | Similarities | Expect |
| Domain | Region | for the Matched Region | Value |
| Ald_Xan_dh_C | 396 . . . 411 | 8/16 (50%) | 0.43 |
| | | 14/16 (88%) | |
| Adenylsucc_synt | 32 . . . 455 | 261/431 (61%) | 0 |
| | | 417/431 (97%) | |

### Example 13

The NOV13 clone was analyzed, and the nucleotide and encoded polypeptide sequences are shown in Table 13A.

TABLE 13A
NOV13 Sequence Analysis

| | |
|---|---|
| SEQ ID NO:65 | 278 bp |

| | |
|---|---|
| NOV13a, | TGCCTGTGGGTTTGATTGCTACCACTCTTGCAATTGCTCCTGTCCGCTTTGACAGGGAGAAGGTGTT |
| CG144686-01 | |
| DNA Sequence | CCGCGTGAAGCCTCAGGATGAAAAACAAGCAGACATCATAAAGGACTTGGCCAAAACCAGTGAGCTC |
| | CGAGATAAAGGCAAATTTGGTTTTCTCCTTCCAGAATCCCGGATAAAGCCAACGTGCAGAGAGACCA |
| | TGCTAGCTGTCAAATTTATTGCCAAGTATATCCTCAAGCATACTTCCTAAAGAACTGCCCTCTGTTT |
| | GGAATAAGCC |

| | | |
|---|---|---|
| ORF Start: at 3 | | ORF Stop: TAA at 249 |
| SEQ ID NO: 66 | 82 aa | MW at 9327.9kD |

| | |
|---|---|
| NOV13a, | PVGLIATTLAIAPVRFDREKVFRVKPQDEKQADIIKDLAKTSELRDKGKFGFLLPESRIKPTCRETM |
| CG144686-01 | |
| Protein Sequence | LAVKFIAKYILKHTS |

| | |
|---|---|
| SEQ ID NO: 67 | 268 bp |

| | |
|---|---|
| NOV13b, | CACCGGATCCACCCCTGTGGGTTTGATTGCTACCACTCTTGCAATTGCTCCTGTCCGCTTTGACAGG |
| 27869008 | |
| DNA Sequence | GAGAAGGTGTTCCGCGTGAAGCCTCAGGATGAAAAACAAGCAGACATCATAAAGGACTTGGCCAAAA |
| | CCAGTGAGCTCCGAGATAAAGGCAAATTTGTTTTCTCCTTCCAGAATCCCGGATAAAGCCAACGTG |
| | CAGAGAGACCATGCTAGCTGTCAAATTTATTGCCAAGTATATCCTCAAGCATACTTCCCTCGAGGGC |

| | | |
|---|---|---|
| ORF Start: at 2 | | ORF Stop: end of sequence |
| SEQ ID NO: 68 | 89 aa | MW at 9973.6kD |

| | |
|---|---|
| NOV13b, | TGSTPVGLIATTLAIAPVRFDREKVFRVKPQDEKQADIIKDLAKTSELRDKGKFGFLLPESRIKPTC |
| 278690008 | |
| Protein Sequence | RETMLAVKFIAKYILKHTSLEG |

| | |
|---|---|
| SEQ ID NO: 69 | 94 bp |

| | |
|---|---|
| NOV13c, | CACCGGATCCACCAGTGAGCTCCGAGATAAAGGCAAATTTGGTTTTCTCCTTCCAGAATCCCGGATA |
| 278690035 | |
| DNA Sequence | AAGCCAACGTGAGAGAGCTCGAGGGC |

| | | |
|---|---|---|
| ORF Start: at 2 | | ORF Stop: end of sequence |
| SEQ ID NO: 70 | 431 aa | MW at 3452.9kD |

| | |
|---|---|
| NOV13c, | TGSTSELRDKGKFGFLLPESRIKPTCRELEG |
| 278690035 | |
| Protein Sequence | |

| | |
|---|---|
| SEQ ID NO:71 | 1622 bp |

| | |
|---|---|
| NOV13d, | ATGAGGCTCATCCTGCCTGTGGGTTTGATTGCTACCACTCTTGCAATTGCTCCTGTCCGCTTTGACA |
| CG144686-02 | |
| DNA Sequence | GGGAGAAGGTGTTCCGCGTGAAGCCCCAGGATGAAAAACAAGCAGACATCATAAAGGACTTGGCCAA |
| | AACCAATGAGCTTGACTTCTGGTATCCAGGTGCCACCCACCACGTAGCTGCTAATATGATGGTGGAT |
| | TTCCGAGTTAGTGAGAAGGAATCCCAAGCCATCCAGTCTGCCTTGGATCAAAATAAAATGCACTATG |
| | AAATCTTGATTCATGATCTACAAGAAGAGATTGAGAAACAGTTTGATGTTAAAGAAGATATCCCAGG |
| | CAGGCACAGCTACGCAAAATACAATAATTGGGAAAAGATTGTGGCTTGGACTGAAAAGATGATGGAT |
| | AAGTATCCTGAAATGGTCTCTCGTATTAAAATTGGATCTACTGTTGAAGATAATCCACTATATGTTC |
| | TGAAGATTGGGGAAAAGAATGAAAGAAGAAAGGCTATTTTTATGGATTGTGGCATTCACGCACGAGA |
| | ATGGGTCTCCCCAGCATTCTGCCAGTGGTTTGTCTATCAGGCAACCAAAACTTATGGGAGAAACAAA |
| | ATTATGACCAAACTCTTGGACCGAATGAATTTTTACATTCTTCCTGTGTTCAATGTTGATGGATATA |
| | CGGCACTGACCTCAACAGGAATTTTAATGCTTCATGGAACTCCATTCCTAACACCAATGACCCATGT |
| | GCAGATAACTATCGGGGCTCTGCACCAGAGTCCGAGAAAGAGACGAAAGCTGTCACTAATTTCATTA |
| | GAAGCCACCTGAATGAAATCAAGGTTTACATCACCTTCCATTCCTACTCCCAGATGCTATTGTTTCC |
| | CTATGGATATACATCAAAACTGCCACCTAACCATGAGGACTTGGCCAAAGTTGCAAAGATTGGCACT |
| | GATGTTCTATCAACTCGATATGAAACCCGCTACATCTATGGCCCAATAGAATCAACAATTTACCCGA |
| | TATCAGGTTCTTCTTTAGACTGGGCTTATGACCTGGGCATCAAACACACATTTGCCTTTGAGCTCCG |
| | AGATAAAGGCAAATTTGGTTTTCTCCTTCCAGAATCCCGGATAAAGCCAACGTGCAGAGAGACCATG |
| | CTAGCTGTCAAATTTATTGCCAAGTATATCCTCAAGCATACTTCCTAAAGAACTGCCCTCTGTTTGG |
| | AATAAGCCAATTAATCCTTTTTTGTGCCTTTCATCAGAAAGTCAATCTTCAGTTATCCCCAAATGCA |
| | GCTTCTATTTCACCTGAATCCTTCTCTTGCTCATTTAAGTCCCATGTTACTGCTGTTTGCTTTTACT |
| | TACTTTCAGTAGCACCATAACGAAGTAGCTTTAAGTGAAACCTTTTAACTACCTTTCTTTGCTCCAA |
| | GTGAAGTTTGGACCCAGCAGAAAGCATTATTTTGAAAGGTGATATACAGTGGGGCACAGAAAACAAA |
| | TGAAAACCCTCAGTTTCTCACAGATTTTCACCATGTGGCTTCATCAATTTATGTGCTAATACAATAA |
| | AATAAAATGCACTT |

| | | |
|---|---|---|
| ORF Start: ATG at 1 | | ORF Stop: TAA at 1252 |
| SEQ ID NO: 72 | 417 aa | MW at 48699.4kD |

| | |
|---|---|
| NOV13d, | MRLILPVGLIATTLAIAPVRFDREKVFRVKPQDEKQADIIKDLAKTNELDFWYPGATHHVAANMMVD |
| CG144686-02 | |
| Protein Sequence | FRVSEKESQAIQSALDQNKMHYEILIHDLQEEIEKQFDVKEDIPGRHSYAKYNNWEKIVAWTEKMMD |
| | KYPEMVSRIKIGSTVEDNPLYVLKIGEKNERRKAIFMDCGIHAREWVSPAFCQWFVYQATKTYGRNK |
| | IMTKLLDRMNFYILPVFNVDGYIWSWTKNRMWRKNRSKNQNSKCIGTDLNRNFNASWNSIPNTNDPC |
| | ADNYRGSAPESEKETKAVTNFIRSHLNEIKVYITFHSYSQMLLFPYGYTSKLPPNHEDLAKVAKIGT |
| | DVLSTRYETRYIYGPIESTIYPISGSSLDWAYDLGIKHTFAFELRDKGKFGFLLPESRIKPTCRETM |
| | LAVKFIAKYILKHTS |

Sequence comparison of the above protein sequences yields the following sequence relationships shown in Table 13B.

TABLE 13B
Comparison of NOV13a against NOV13b through NOV13d.

| Protein | NOV13a Residues/ | Identities/Similarities |
|---|---|---|
| Sequence | Match Residues | for the Matched Region |
| NOV13b | 1 . . . 82 | 82/82 (100%) |
| | 5 . . . 86 | 82/82 (100%) |
| NOV13c | 41 . . . 65 | 25/25 (100%) |
| | 4 . . . 28 | 25/25 (100%) |
| NOV13d | 1 . . . 44 | 43/44 (97%) |
| | 6 . . . 49 | 44/44 (99%) |

Further analysis of the NOV13a protein yielded the following properties shown in Table 13C.

TABLE 13C
Protein Sequence Properties NOV13a

| | |
|---|---|
| PSort analysis: | 0.5500 probability located in endoplasmic reticulum |
| | (membrane); 0.1900 probability located in lysosome |
| | (lumen); 0.1000 probability located in endoplasmic |
| | reticulum (lumen); 0.1000 probability located in |
| | outside |
| SignalP analysis: | No Known Signal Sequence Predicted |

A search of the NOV13a protein against the Geneseq database, a proprietary database that contains sequences published in patents and patent publication, yielded several homologous proteins shown in Table 13D.

TABLE 13D
Geneseq Results for NOV13a

| | | | Identities/ | |
|---|---|---|---|---|
| | | | Similarities for | |
| Geneseq | Protein/Organism/Length | NOV13a Residues/ | the Matched | Expect |
| Identifier | [Patent #, Date] | Match Residues | Region | Value |
| AAU84325 | Protein CPA3 differentially | 1 . . . 44 | 43/44 (97%) | 2e−17 |
| | expressed in breast cancer | 6 . . . 49 | 44/44 (99%) | |
| | tissue - Homo sapiens, 417 | | | |
| | aa. [WO200210436-A2, | | | |
| | 07-FEB-2002] | | | |
| AAG75369 | Human colon cancer antigen | 43 . . . 82 | 40/40 (100%) | 9e−17 |
| | protein SEQ ID NO: 6133 - | 141 . . . 180 | 40/40 (100%) | |
| | Homo sapiens, 180 aa. | | | |
| | [WO200122920-A2, | | | |
| | 05-APR-200] | | | |
| AAU04477 | Porcine carboxypeptidase B | 41 . . . 80 | 25/40 (62%) | 4e−10 |
| | (CpB) protein - Sus scrofa, | 266 . . . 305 | 34/40 (84%) | |
| | 306 aa. [WO200151624-A2, | | | |
| | 19-JUL-2001] | | | |
| AAR75132 | Porcine carboxypeptidase B - | 41 . . . 80 | 25/40 (62%) | 4e−10 |
| | Sus scrofa, 306 aa. | 266 . . . 305 | 34/40 (84%) | |
| | [WO9514096-A1, | | | |
| | 26-MAY-1995] | | | |
| AAR75131 | Porcine Tyr-His-Met | 41 . . . 80 | 25/40 (62%) | 4e−10 |
| | Procarboxypeptidase B - Sus | 364 . . . 403 | 34/40 (84%) | |
| | scrofa, 404 aa. | | | |
| | [WO9514096-A1, | | | |
| | 26-MAY-1995] | | | |

In a BLAST search of public sequence datbases, the NOV 13a protein was found to have homology to the proteins shown in the BLASTP data in Table 13E.

TABLE 13E
Public BLASTP Results for NOV13a

| | | | Identities/ | |
|---|---|---|---|---|
| Protein | | | Similarities for | |
| Accession | | NOV13a Residues/ | the Matched | Expect |
| Number | Protein/Organism/Length | Match Residues | Portion | Value |
| P15088 | Mast cell carboxypeptidase A | 1 . . . 44 | 43/44 (97%) | 5e−17 |
| | precursor (EC 3.4.17.1) | 6 . . . 49 | 44/44 (99%) | |
| | (MC-CPA) (Carboxypeptidase | | | |
| | A3) - Homo sapiens (Human), | | | |
| | 417 aa. | | | |
| P97597 | Mast cell carboxypeptidase A | 43 . . . 82 | 37/40 (92%) | 1e−14 |
| | precursor - Rattus norvegicus | 373 . . . 412 | 39/40 (97%) | |
| | (Rat), 412 aa (fragment). | | | |
| P21961 | Mast cell carboxypeptidase | 43 . . . 82 | 37/40 (92%) | 1e−14 |
| | (EC 3.4. 17.1) (RMC-CP) | 270 . . . 309 | 39/40 (97%) | |
| | (Carboxypeptidase A3) - Rattus | | | |
| | norvegicus (Rat), 309 aa. | | | |
| P15089 | Mast cell carboxypeptidase A | 43 . . . 82 | 36/40 (90%) | 7e−14 |
| | precursor (EC 3.4.17.1) | 378 . . . 417 | 39/40 (97%) | |
| | (MC-CPA) (Carboxypeptidase | | | |
| | A3) - Mus musculus (Mouse), | | | |
| | 417 aa. | | | |
| P00732 | Carboxypeptidase B (EC | 41 . . . 80 | 26/40 (65%) | 7e−11 |
| | 3.4.17.2) - Bos taurus (Bovine), | 266 . . . 305 | 36/40 (90%) | |
| | 306 aa. | | | |

PFam analysis predicts that the NOV13a protein contains the domains shown in the Table 13F.

TABLE 13F
Domain Analysis of NOV13a

| | | Identities/ | |
|---|---|---|---|
| | | Similarities | |
| Pfam | NOV13a | for the Matched | Expect |
| Domain | Match Region | Region | Value |
| Zn_carbOpept | 41 . . . 65 | 16/30 (53%) | 5.6e−08 |
| | | 24/30 (80%) | |

### Example 14

The NOV14 clone was analyzed, and the nucleotide and encoded polypeptide sequences are shown in Table 14A.

TABLE 14A
NOV14 Sequence Analysis

| | |
|---|---|
| SEQ ID NO: 73 | 829 bp |

| | |
|---|---|
| NOV14a, | GCCCTTCGCGGGAGAGGAGGCCATGGGCGCGCGCGGGGCGCTGCTGCTGGCGCTGCTGCTGGCTCGG |
| CG144906-01 | |
| DNA Sequence | GCTGGACTCAGGAAGCCGGAGTCGCAGGAGGCGGCGCCCTTATCAGGACCATGCGGCCGACGGGTCA |
| | TCACGTCGCGCATCGTGGGTGGAGAGGACGCCGAACTCGGGCGTTGGCCGTGGCAGGGGAGCCTGCG |
| | CCTGTGGGATTCCCACGTATGCGGAGTGAGCCTGCTCAGCCACCGCTGGGCACTCACGGCGGCGCAC |
| | TGCTTTGAAACCTATAGTGACCTTAGTGATCCCTCCGGGTGGATGGTCCAGTTTGGCCAGCTGACTT |
| | CCATGCCATCCTCCACATTTGAGTTTGAGAACCGGACAGACTGCTGGGTGACTGGCTGGGGGTACAT |
| | CAAAGAGGATGAGGCACTGCCATCTCCCCACACCCTCCAGGAAGTTCAGGTCGCCATCATAAACAAC |
| | TCTATGTGCAACCACCTCTTCCTCAAGTACAGTTTCCGCAAGGACATCTTTGGAGACATGGTTTGTG |
| | CTGGCAATGCCCAAGGCGGGAAGGATGCCTGCTTCGGTGACTCAGGTGGACCCTTGGCCTGTAACAA |
| | GAATGGACTGTGGTATCAGATTGGAGTCGTGAGCTGGGGAGTGGGCTGTGGTCGGCCCAATCGGCCC |
| | GGTGTCTACACCAATATCAGCCACCACTTTGAGTGGATCCAGAAGCTGATGGCCCAGAGTGGCATGT |
| | CCCAGCCAGACCCCTCCTGGCCACTACTCTTTTTCCCTCTTCTCTGGGCTCTCCCACTCCTGGGGCC |
| | GGTCTGAGCCTACCTGAGCCCATGC |

| | | |
|---|---|---|
| ORF Start: ATG at 23 | | ORF Stop: TGA at 809 |
| SEQ ID NO: 74 | 262 aa | MW at 28826.7kD |

| | |
|---|---|
| NOV14a, | MGARGALLLALLLARAGLRKPESQEAAPLSGPCGRRVITSRIVGGEDAELGRWPWQGSLRLWDSHVC |
| CG144906-01 | |
| Protein Sequence | GVSLLSHRWALTAAHCFETYSDLSDPSGWMVQFGQLTSMPSSTFEFENRTDCWVTGWGYIKEDEALP |
| | SPHTLQEVQVAIINNSMCNHLFLKYSFRKDIFGDMVCAGNAQGGKDACFGDSGGPLACNKNGLWYQI |
| | GVVSWGVGCGRPNRPGVYTNISHHFEWIQKLMAQSGMSQPDPSWPLLFFPLLWALPLLGPV |

| | |
|---|---|
| SEQ ID NO: 75 | 989 bp |

| | |
|---|---|
| NOV14b, | AATCGCCCTTCGCGGGAGAGGAGGCCATGGGCGCGCGCGGGGCGCTGCTGCTGGCGCTGCTGCTGGC |
| CG144906-02 | |
| DNA Sequence | TCGGGCTGGACTCAGGAAGCCGGAGTCGCAGGAGGCGGCGCCCTTATCAGGACCATGCGGCCGACGG |
| | GTCATCACGTCGCGCATCGTGGGTGGAGAGGACGCCGAACTCGGGCGTTGGCCGTGGCAGGGGAGCC |
| | TGCGCCTGTGGGATTCCCACGTATGCGGAGTGAGCCTGCTCAGCCACCGCTGGGCACTCACGGCGGC |
| | GCACTGCTTTGAAACCTATAGTGACCTTAGTGATCCCTCCGGGTGGATGGTCCAGTTTGGCCAGCTG |
| | ACTTCCATGCCATCCTTCTGGAGCCTGCAGGCCTACTACACCCGTTACTTCGTATCGAATATCTATC |
| | TGAGCCCTCGCTACCTGGGGAATTCACCCTATGACATTGCCTTGGTGAAGCTGTCTGCACCTGTCAC |
| | CTACACTAAACACATCCAGCCCATCTGTCTCCAGGCCTCCACATTTGAGTTTGAGAACCGGACAGAC |
| | TGCTGGGTGACTGGCTGGGGGTACATCAAAGAGGATGAGGCACTGCCATCTCCCCACACCCTCCAGG |
| | AAGTTCAGGTCGCCATCATAAACAACTCTATGTGCAACCACCTCTTCCTCAAGTACAGTTTCCGCAA |
| | GGACATCTTTGGAGACATGGTTTGTGCTGGCAATGCCCAAGGCGGGAAGGATGCCTGCTTCGGTGAC |
| | TCAGGTGGACCCTTGGCCTGTAACAGGAATGGACTGTGGTATCAGATTGGAGTCGTGAGCTGGGGAG |
| | TGGGCTGTGGTCGGCCCAATCGGCCCGGTGTCTACACCAATATCAGCCACCACTTTGAGTGGATCCA |
| | GAAGCTGATGGCCCAGAGTGGCATGTCCCAGCCAGACCCCTCCTGGCCACTACTCTTTTTCCCTCTT |
| | CTCTGGGCTCTCCCACTCCTGGGGCCGGTCTGAGCCTACCTTAGCCCATGC |

| | | |
|---|---|---|
| ORF Start: ATG at 27 | | ORF Stop: TGA at 969 |
| SEQ ID NO: 76 | 314 aa | MW at 34911.6kD |

| | |
|---|---|
| NOV14b, | MGARGALLLALLLARAGLRKPESQEAAPLSGPCGRRVITSRIVGGEDAELGRWPWQGSLRLWDSHVC |
| CG144906-02 | |
| Protein Sequence | GVSLLSHRWALTAAHCFETYSDLSDPSGWMVQFGQLTSMPSFWSLQAYYTRYFVSNIYLSPRYLGNS |
| | PYDIALVKLSAPVTYTKHIQPICLQASTFEFENRTDCWVTGWGYIKEDEALPSPHTLQEVQVAIINN |
| | SMCNHLFLKYSFRKDIFGDMVCAGNAQGGKDACFGDSGGPLACNRNGLWYQIGVVSWGVGCGRPNRP |
| | GVYTHISHHFEWIQKLMAQSGMSQPDPSWPLLFFPLLWALPLLGPV |

Sequence comparison of the above protein sequences yields the following sequence relationships shown in Table 14B.

TABLE 14B
Comparison of NOV14a against NOV14b.

| | | Identities/ |
|---|---|---|
| | NOV14a Residues/ | Similarities for |
| Protein Sequence | Match Residues | the Matched Region |
| NOV14b | 20 . . . 240 | 219/273 (80%) |
| | 20 . . . 292 | 221/273 (80%) |

Further analysis of the NOV14a protein yielded the following properties shown in Table 14C.

TABLE 14C
Protein Sequence Properties NOV14a

| | |
|---|---|
| PSort analysis: | 0.5422 probability located in outside; 0.4639 probability located in lysosome |
| | (lumen); 0.2779 probability located in microbody (peroxisome); 0.1900 |
| | probability located in plasma membrane |
| SignalP analysis: | Cleavage site between residues 20 and 21 |

A search of the NOV14a protein against the Geneseq database, a proprietary database that contains sequences published in patents and patent publication, yielded several homologous proteins shown in Table 14D.

TABLE 14D
Geneseq Results for NOV14a

| | | NOV14a | | |
|---|---|---|---|---|
| | | Residues/ | Identities/ | |
| Geneseq | Protein/Organism/Length | Match | Similarities for | Expect |
| Identifier | [Patent #, Date] | Residues | the Matched Region | Value |
| AAE17010 | Human eosinophil serine | 1 . . . 262 | 262/314 (83%) | e−154 |
| | protease-1 (esp-1) like | 1 . . . 314 | 262/314 (83%) | |
| | enzyme #2 - Homo sapiens, | | | |
| | 314 aa.[WO200198503-A2, | | | |
| | 27-DEC-2001] | | | |
| AAB80256 | Human PR0303 protein - | 1 . . . 262 | 262/314 (83%) | e−154 |
| | Homo sapiens, 314 aa. | 1 . . . 314 | 262/314 (83%) | |
| | [WO200104311-A1, | | | |
| | 18-JAN-2001] | | | |
| AAU01569 | Human secreted protein | 1 . . . 262 | 262/314 (83%) | e−154 |
| | immunogenic epitope | 1 . . . 314 | 262/314 (83%) | |
| | encoded by gene #9 - Homo | | | |
| | sapiens, 315 aa. | | | |
| | [WO200123547-A1, | | | |
| | 05-APR-2001] | | | |
| AAU02223 | Human extracellular serine | 1 . . . 262 | 262/314 (83%) | e−154 |
| | protease TADG-16 - Homo | 1 . . . 314 | 262/314 (83%) | |
| | sapiens, 314 aa. | | | |
| | [WO200127257-A1, | | | |
| | 19-APR-2001] | | | |
| AAY91871 | Human cancer-specific gene | 1 . . . 262 | 262/314 (83%) | e−154 |
| | protein, Pro104 - Homo | 14 . . . 327 | 262/314 (83%) | |
| | sapiens, 327 aa. | | | |
| | [WO200016805-A1, | | | |
| | 30-MAR-2000] | | | |

In a BLAST search of public sequence datbases, the NOV14a protein was found to have homology to the proteins shown in the BLASTP data in Table 14E.

TABLE 14E
Public BLASTP Results for NOV14a

| | | NOV14a | | |
|---|---|---|---|---|
| Protein | | Residues/ | Identities/ | |
| Accession | | Match | Similarities for | Expect |
| Number | Protein/Organism/Length | Residues | the Matched Portion | Value |
| Q9Y6MO | Testisin precursor (EC | 1 . . . 262 | 262/314 (83%) | e−154 |
| | 3.4.21.-) (Eosinophil serine | 1 . . . 314 | 262/314 (83%) | |
| | protease 1) (ESP-1) - Homo | | | |
| | sapiens (Human), 314 aa. | | | |
| Q9JHJ7 | Testisin precursor (EC | 1 . . . 261 | 179/326 (54%) | 1e−98 |
| | 3.4.21.-) (Tryptase 4) - Mus | 1 . . . 323 | 210/326 (63%) | |
| | musculus (Mouse), 324 aa. | | | |
| Q920S2 | Testis serine protease-1 - Mus | 1 . . . 261 | 150/325 (46%) | 2e−69 |
| | musculus (Mouse), 322 aa. | 1 . . . 321 | 180/325 (55%) | |
| Q9D4I3 | 493l44OBO9Rik protein - | 32 . . . 261 | 135/283 (47%) | 1e−66 |
| | Mus musculus (Mouse), 282 | 2 . . . 281 | 161/283 (56%) | |
| | aa. | | | |
| Q9PVX7 | Epidermis specific serine | 33 . . . 244 | 100/264 (37%) | 3e−45 |
| | protease - Xenopus Laevis | 17 . . . 277 | 136/264 (50%) | |
| | (African clawed frog), 389 aa. | | | |

PFam analysis predicts that the NOV14a protein contains the domains shown in the Table 14F.

TABLE 14F
Domain Analysis of NOV14a

| | | Identities/ | |
|---|---|---|---|
| Pfam | | Similarities for | Expect |
| Domain | NOV14a Match Region | the Matched Region | Value |
| trypsin | 42 . . . 85 | 24/51 (47%) | 2.3e−13 |
| | | 36/51 (71%) | |
| trypsin | 119 . . . 229 | 52/121 (43%) | 9e−43 |
| | | 92/121 (76%) | |

### Example 15

The NOV15 clone was analyzed, and the nucleotide and encoded polypeptide sequences are shown in Table 15A.

TABLE 15A
NOV15 Sequence Analysis

| | |
|---|---|
| SEQ ID NO:77 | 716 bp |

| | |
|---|---|
| NOV15a, | GAGTGAGCGATGAGCTGGTTTCTGTTCCTGGCCCACAGAGTCGCCTTGGCCGCCTTGCCCTGCCGCC |
| CG144997-01 | |
| DNA Sequence | GCGGCTCTCGCGGGTTCGGGATGTTCTATGCCGTGAGGAGGGGCCGCAAGACCGGGGTCTTTCTGAC |
| | CTGGAATGAGTGCAGAGACACGTTTTCCTACATGGGAGACTTCGTCGTCGTCTACACTGATGGCTGC |
| | TGCTCCAGTAATGGGCGTAGAAGGCCGCGAGCAGGAATCGGCGTTTACTGGGGGCCGGGCCATCCTT |
| | TAAATGTAGGCATTAGACTTCCTGGGCGGCAGACAAACCAAAGAGCGGAAATTCATGCAGCCTGCAA |
| | AGCCATTGAACAAGCAAAGACTCAAAACATCAATAAACTGGTTCTGTATACAGACAGTATGTTTACG |
| | ATAAATGGTATAACTAACTGGGTTCAAGGTTGGAAGAAAAATGGGTGGAAGACAAGTGCAGGGAAAG |
| | AGGTGATCAACAAAGAGGACTTTGTGGCACTGGAGAGGCTTACCCAGGGGATGGACATTCAGTGGAT |
| | GCATGTTCCTGGTCATTCGGGATTTATAGGCAATGAAGAAGCTGACAGATTAGCCAGAGAAGGAGCT |
| | AAACAATCGGAAGACTGAGCCATGTGACTTTAGTCCTTGGGAGAACTTGAGCCAGCGGCTGTCTTGC |
| | TGCCTGTACTTACTGGTGTGGAAAATAGCCTGCAGGTAGGACCATT |

| | | |
|---|---|---|
| ORF Start: ATG at 10 | | ORF Stop: TGA at 619 |
| SEQ ID NO: 78 | 203 aa | MW at 22889.0 kD |

| | |
|---|---|
| NOV15a, | MSWFLFLAHRVALAALPCRRGSRGFGMFYAVRRGRKTGVFLTWNECRDTFSYMGDFVVVYTDGCCSS |
| CG144997-01 | |
| Protein Sequence | NGRRRPRAGIGVYWGPGHPLNVGIRLPGRQTNQRAEIHAACKAIEQAKTQNINKLVLYTDSMFTING |
| | ITNWVQGWKKNGWKTSAGKEVINKEDFVALERLTQGMDIQWMHVPGHSGFIGNEEADRLAREGAKQS |
| | ED |

| | |
|---|---|
| SEQ ID NO: 79 | 631 bp |

| | |
|---|---|
| NOV15b, | CACCGGATCCACCATGAGCTGGTTTCTGTTCCTGGCCCACAGAGTCGCCTTGGCCGCCTTGCCCTGC |
| 278693648 | |
| DNA Sequence | CGCCGCGGCTCTCGCGGGTTCGGGATGTTCTATGCCGTGAGGAGGGGCCGCAAGACCGGGGTCTTTC |
| | TGACCTGGAATGAGTGCAGAGACACGTTTTCCTACATGGGAGACTTCGTCGTCGTCTACACTGATGG |
| | CTGCTGCTCCAGTAATGGGCGTAGAAGGCCGCGAGCAGGAATCGGCGTTTACTGGGGGCCGGGCCAT |
| | CCTTTAAATGTAGGCATTAGACTTCCTGGGCGGCAGACAAACCAAAGAGCGGAAATTCATGCAGCCT |
| | GCAAAGCCATTGAACAAGCAAAGACTCAAAACATCAATAAACTGGTTCTGTATACAGACAGTATGTT |
| | TACGATAAATGGTATAACTAACTGGGTTCAAGGTTGGAAGAAAAATGGGTGGAAGACAAGTGCAGGG |
| | AAAGAGGTGATCAACAAAGAGGACTTTGTGGCACTGGAGAGGCTTACCCAGGGGATGGACATTCAGT |
| | GGATGCATGTTCCTGGTCATTCGGGATTTATAGGCAATGAAGAAGCTGACAGATTAGCCAGAGAAGG |
| | AGCTAAACAATCGGAAGACCTCGAGGGC |

| | | |
|---|---|---|
| ORF Start: at 2 | | ORF Stop: end of sequence |
| SEQ ID NO: 80 | 210 aa | MW at 23534.6 kD |

| | |
|---|---|
| NOV15b, | TGSTMSWFLFLAHRVALAALPCRRGSRGFGMFYAVRRGRKTGVFLTWNECRDTFSYMGDFVVVYTDG |
| 278693648 | |
| Protein Sequence | CCSSNGRRRPRAGIGVYWGPGHPLNVGIRLPGRQTNQRAEIHAACKAIEQAKTQNINKLVLYTDSMF |
| | TINGITNWVQGWKKNGWKTSAGKEVINKEDFVALERLTQGMDIQWMHVPGHSGFIGNEEADRLAREG |
| | AKGSEDLEG |

| | |
|---|---|
| SEQ ID NO: 81 | 586 bp |

| | |
|---|---|
| NOV15c, | CACCGGATCCGCCTTGCCCTGCCGCCGCGGCTCTCGCGGGTTCGGGATGTTCTATGCCGTGAGGAGG |
| 278480974 | |
| DNA Sequence | GGCCGCAAGACCGGGGTCTTTCTGACCTGGAATGAGTGCAGAGACACGTTTTCCTACATGGGAGACT |
| | TCGTCGTCGTCTACACTGATGGCTGCTGCTCCAGTAATGGGCGTAGAAGGCCGCGAGCAGGAATCGG |
| | CGTTTACTGGGGGCCGGGCCATCCTTTAAATGTAGGCATTAGACTTCCTGGGCGGCAGACAAACCAA |
| | AGAGCGGAAATTCATGCAGCCTGCAAAGCCATTGAACAAGCAAAGACTCAAAACATCAATAAACTGG |
| | TTCTGTATACAGACAGTATGTTTACGATAAATGGTATAACTAACTGGGTTCAAGGTTGGAAGAAAAA |
| | TGGGTGGAAGACAAGTGCAGGGAAAGAGGTGATCAACAAAGAGGACTTTGTGGCACTGGAGAGGCTT |
| | ACCCAGGGGATGGACATTCAGTGGATGCATGTTCCTGGTCATTCGGGATTTATAGGCAATGAAGAAG |
| | CTGACAGATTAGCCAGAGAAGGAGCTAAACAATCGGAAGACCTCGAGGGC |

| | | |
|---|---|---|
| ORF Start: at 2 | | ORF Stop: end of sequence |
| SEQ ID NO: 82 | 195 aa | MW at 21789.5 kD |

| | |
|---|---|
| NOV15c, | TGSALPCRRGSRGFGMFYAVRRGRKTGVFLTWNECRDTFSYMGDFVVVYTDGCCSSNGRRRPRAGIG |
| 2784980974 | |
| Protein Sequence | VYWGPGHPLNVGIRLPGRQTNQRAEIHAACKAIEQAKTQNINKLVLYTDSMFTINGITNWVQGWKKN |
| | GWKTSAGKEVINKEDFVALERLTQGMDIQWMHVPGHSGFIGNEEADRLAREGAKQSEDLEG |

| | |
|---|---|
| SEQ ID NO: 83 | 457 bp |

| | |
|---|---|
| NOV15d, | CAACGGATCCGGAGACTTCGTCGTCGTCTACACTGATGGCTGCTGCTCCAGTAATGGGCGTAGAAGG |
| 278498047 | |
| DNA Sequence | CCGCGAGCAGGAATCGGCGTTTACTGGGGGCCGGGCCATCCTTTAAATGTAGGCATTAGACTTCCTG |
| | GGCGGCAGACAAACCAAAGAGCGGAAATTCATGCAGCCTGCAAAGCCATTGAACAAGCAAAGACTCA |
| | AAACATCAATAAACTGGTTCTGTATACAGACAGTATGTTTACGATAAATGGTATAACTAACTGGGTT |
| | CAAGGTTGGAAGAAAAATGGGTGGAAGACAAGTGCAGGGAAAGAGGTGATCAACAAAGAGGACTTTG |
| | TGGCACTGGAGAGGCTTACCCAGGGGATGGACATTCAGTGGATGCATGTTCCTGGTCATTCGGGATT |
| | TATAGGCAATGAAGAAGCTGACAGATTAGCCAGAGAAGGAGCTAAACTCGAGGGC |

| | | |
|---|---|---|
| ORF Start: at 2 | | ORF Stop: end of sequence |
| SEQ ID NO: 84 | 152 aa | MW at 16753.8 kD |

| | |
|---|---|
| NOV15d, | TGSGDFVVVYTDGCCSSNGRRRPRAGIGVYWGPGHPLNVGIRLPGRQTNQRAEIHAACKAIEQAKTQ |
| 278498047 | |
| Protein Sequence | NINKLVLYTDSMFTINGITNWVQGWKKNGWKTSAGKEVINKEDFVALERLTQGMDIQWMHVPGHSGF |
| | IGNEEADRLAREGAKLEG |

| | |
|---|---|
| SEQ ID NO: 85 | 965 bp |

| | |
|---|---|
| NOV15e, | GAGTGAGCGATGAGCTGGTTTCTGTTCCTGGCCCACAGAGTCGCCTTGGCCGCCTTGCCCTGCCGCC |
| CG144997-02 | |
| DNA Sequence | GCGGCTCTCGCGGGTTCGGGATGTTCTATGCCGTGAGGAGGGGCCGCAAGACCGGGGTCTTTCTGAC |
| | CTGGAATGAGTGCAGAGCACAGGTGGACCGGTTTCCTGCTGCCAGATTTAAGAAGTTTGCCACAGAG |
| | GATGAGGCCTGGGCCTTTGTCAGGAAATCTGCAAGCCCGGAAGTTTCAGAAGGGCATGAAAATCAAC |
| | ATGGACAAGAATCGGAGGCGAAAGCCAGCAAGCGACTCCGTGAGCCACTGGATGGAGATGGACATGA |
| | AAGCGCAGAGCCGTATGCAAAGCACATGAAGCCGAGCGTGGAGCCGGCGCCTCCAGTTAGCAGAGAC |
| | ACGTTTTCCTACATGGGAGACTTCGTCGTCGTCTACACTGATGGCTGCTGCTCCAGTAATGGGCGTA |
| | GAAGGCCGCGAGCAGGAATCGGCGTTTACTGGGGGCCAGGCCATCCTTTAAATGTAGGCATTAGACT |
| | TCCTGGGCGGCAGACAAACCAAAGAGCGGAAATTCATGCAGCCTGCAAAGCCATTGAACAAGCAAAG |
| | ACTCAAAACATCAATAAACTGGTTCTGTATACAGACAGTATGTTTACGATAAATGGTATAACTAACT |
| | GGGTTCAAGGTTGGAAGAAAAATGGGTGGAAGACAAGTGCAGGGAAAGAGGTGATCAACAAAGAGGA |
| | CTTTGTGGCACTGGAGAGGCTTACCCAGGGGATGGACATTCAGTGGATGCATGTTCCTGGTCATTCG |
| | GGATTTATAGGCAATGAAGAAGCTGACAGATTAGCCAGAGAAGGAGCTAAACAATCGGAAGACTGAG |
| | CCATGTGACTTTAGTCCTTGGGAGAACTTGAGCCAGCGGCTGTCTTGCTGCCTGTACTTACTGGTGT |
| | GGAAAATAGCCTGCAGGTAGGACCATT |

| | | |
|---|---|---|
| ORF Start: ATG at 10 | | ORF Stop: TGA at 868 |
| SEQ ID NO:86 | 286aa | MW at 32098.0kD |

| | |
|---|---|
| NOV15e, | MSWFLFLAHRVALAALPCRRGSRGFGMFYAVRRGRKTGVFLTWNECRAQVDRFPAARFKKFATEDEA |
| CG144997-02 | |
| Protein Sequence | WAFVRKSASPEVSEGHENQHGQESEAKASKRLREPLDGDGHESAEPYAKHMKPSVEPAPPVSRDTFS |
| | YMGDFVVVYTDGCCSSNGRRRPRAGIGVYWGPGHPLNVGIRLPGRQTNQRAEIHAACKAIEQAKTQN |
| | INKLVLYTDSMFTINGITNWVQGWKKNGWKTSAGKEVINKEDFVALERLTQGMDIQWMHVPGHSGFI |
| | GNEEADRLAREGAKQSED |

Sequence comparison of the above protein sequences yields the following sequence relationships shown in Table 15B.

TABLE 15B
Comparison of NOV15a against NOV15b through NOV15e.

| | | Identities/ |
|---|---|---|
| | NOV15a Residues/ | Similarities for |
| Protein Sequence | Match Residues | the Matched Region |
| NOV15b | 1 . . . 203 | 203/203 (100%) |
| | 5 . . . 207 | 203/203 (100%) |
| NOV15c | 14 . . . 203 | 189/190 (99%) |
| | 3 . . . 192 | 190/190 (99%) |
| NOV15d | 54 . . . 199 | 146/146 (100%) |
| | 4 . . . 149 | 146/146 (100%) |
| NOV15e | 47 . . . 203 | 157/157 (100%) |
| | 130 . . . 286 | 157/157 (100%) |

Further analysis of the NOV15a protein yielded the following properties shown in Table 15C.

TABLE 15C
Protein Sequence Properties NOV15a

| | |
|---|---|
| PSort analysis: | 0.3700 probability located in outside; 0.1805 probability located in microbody |
| | (peroxisome); 0.1080 probability located in nucleus; 0.1000 probability |
| | located in endoplasmic reticulum (membrane) |
| SignalP analysis: | Cleavage site between residues 15 and 16 |

A search of the NOV15a protein against the Geneseq database, a proprietary database that contains sequences published in patents and patent publication, yielded several homologous proteins shown in Table 15D.

TABLE 15D
Geneseq Results for NOV15a

| | | NOV15a | | |
|---|---|---|---|---|
| | | Residues/ | Identities/ | |
| Geneseq | Protein/Organism/Length | Match | Similarities for | Expect |
| Identifier | [Patent #, Date] | Residues | the Matched Region | Value |
| AAY70235 | Human RNA-associated | 47 . . . 203 | 157/157 (100%) | 6e−92 |
| | protein-16 (RNAAP-16) - | 130 . . . 286 | 157/157 (100%) | |
| | Homo sapiens, 286 aa. | | | |
| | [WO200011171-A2, | | | |
| | 02-MAR-2000] | | | |
| AAB97508 | Human type II RNase H | 47 . . . 203 | 156/157 (99%) | 1e−91 |
| | protein - Homo sapiens, 286 | 130 . . . 286 | 157/157 (99%) | |
| | aa. [WPO200123613-A1, | | | |
| | 05-APR-2001] | | | |
| AAY25094 | Human type 2 RNase H | 47 . . . 203 | 156/157 (99%) | 1e−91 |
| | protein - Homo sapiens, 286 | 130 . . . 286 | 157/157 (99%) | |
| | aa. [WO9928447-A1, | | | |
| | 10-JUN-1999] | | | |
| ABB83371 | Human wild-type RNase H1 - | 47 . . . 203 | 156/157 (99%) | 2e−90 |
| | Homo sapiens, 286 aa. | 130 . . . 286 | 156/157 (99%) | |
| | [WO200240635-A2, | | | |
| | 23-MAY-2002] | | | |
| ABB83374 | Mutant RNase H1, E186Q - | 47 . . . 203 | 155/157 (98%) | 5e−90 |
| | Homo sapiens, 286 aa. | 130 . . . 286 | 156/157 (98%) | |
| | [WO200240635-A2, | | | |
| | 23-MAY-2002] | | | |

In a BLAST search of public sequence datbases, the NOV15a protein was found to have homology to the proteins shown in the BLASTP data in Table 15E.

TABLE 15E
Public BLASTP Results for NOV15a

| | | NOV15a | | |
|---|---|---|---|---|
| Protein | | Residues/ | Identities/ | |
| Accession | | Match | Similarities for the | Expect |
| Number | Protein/Organism/Length | Residues | Matched Portion | Value |
| O60930 | Ribonuclease H1 (EC | 47 . . . 203 | 157/157 (100%) | 2e−91 |
| | 3.1.26.4) (RNase H1) | 130 . . . 286 | 157/157 (100%) | |
| | (Ribonuclease H type II) - | | | |
| | Homo sapiens (Human), 286 | | | |
| | aa. | | | |
| Q8VCR6 | Ribonuclease H1 - Mus | 47 . . . 203 | 139/157 (88%) | 5e−83 |
| | musculus (Mouse), 285 aa. | 129 . . . 285 | 150/157 (95%) | |
| O70338 | Ribonuclease H1 (EC | 47 . . . 203 | 139/157 (88%) | 5e−83 |
| | 3.1.26.4) (RNase H1) - Mus | 129 . . . 285 | 150/157 (95%) | |
| | musculus (Mouse), 285 aa. | | | |
| Q91953 | mRNA, complete cds, clone | 50 . . . 202 | 117/153 (76%) | 4e−70 |
| | CLFEST65 - Gallus gallus | 140 . . . 292 | 135/153 (87%) | |
| | (Chicken), 293 aa. | | | |
| Q21024 | F59A6.6 protein - | 58 . . . 199 | 65/142 (45%) | 3e−32 |
| | Caenorhabditis elegans, 369 | 222 . . . 363 | 93/142 (64%) | |
| | aa. | | | |

PFam analysis predicts that the NOV15a protein contains the domains shown in the Table 15F.

TABLE 15F
Domain Analysis of NOV15a

| | | Identities/ | |
|---|---|---|---|
| Pfam | | Similarities | Expect |
| Domain | NOV15a Match Region | for the Matched Region | Value |
| rnaseH | 54 . . . 199 | 65/176 (37%) | 2.8e−54 |
| | | 125/176 (71%) | |

### Example 16

The NOV16 clone was analyzed, and the nucleotide and encoded polypeptide sequences are shown in Table 16A.

TABLE 16A
NOV16 Sequence Analysis

| | SEQ ID NO: 87 | 2274 bp | |
|---|---|---|---|
| NOV 16a, | CCCCTAGTGACACTCAGGAAATGCTTGTCTCCGGCTGTTAAGGAATAATTTCAGAGTACTATGGATC | | |
| CG145494-01 | | | |
| DNA Sequence | ATGCTGAAGAAAATGAAATCCTTGCAGCAACCCAGAGGTACTATGTGGAAAGGCCTATCTTTAGTCA | | |
| | TCCGGTCCTCCAGGAAAGACTACACACAAAGGACAAGGTTCCTGATTCCATTGCGGATAAGCTGAAA | | |
| | CAGGCATTCACATGTACTCCTAAAAAAATAAGAAATATCATTTATATGTTCCTACCCATAACTAAAT | | |
| | GGCTGCCAGCATACAAATTCAAGGAATATGTGTTGGGTGACTTGGTCTCAGGCATAAGCACAGGGGT | | |
| | GCTTCAGCTTCCTCAAGGCTTAGCCTTTGCAATGCTGGCAGCTGTGCCTCCAATATTTGGCCTGTAC | | |
| | TCTTCATTTTACCCTGTTATCATGTATTGTTTTCTTGGAACCTCCAGACACATATCCATAGGTCCTT | | |
| | TTGCTGTTATTAGCCTGATGATTGGTGGTGTAGCTGTTCGATTAGTACCAGATGATATAGTCATTCC | | |
| | AGGAGGAGTAAATGCAACCAATGGCACAGAGGCCAGAGATGCCTTGAGAGTGAAAGTCGCCATGTCT | | |
| | GTGACCTTACTTTCAGGAATCATTCAGTTTTGCCTAGGTGTCTGTAGGTTTGGATTTGTGGCCATAT | | |
| | ATCTCACAGAGCCTCTGGTCCGTGGGTTTACCACCGCAGCAGCTGTGCATGTCTTCACCTCCATGTT | | |
| | AAAATATCTGTTTGGAGTTAAAACAAAGCGGTACAGTGGAATCTTTTCCGTGGTGTATAGTACAGTT | | |
| | GCTGTGTTGCAGAATGTTAAAAACCTCAACGTGTGTTCCCTAGGCGTCGGGCTGATGGTTTTTGGTT | | |
| | TGCTGTTGGGTGGCAAGGAGTTTAATGAGAGATTTAAAGAGAAATTGCCGGCGCCTATTCCTTTAGA | | |
| | GTTCTTTGCGGTCGTAATGGGAACTGGCATTTCAGCTGGGTTTAACTTGAAAGAATCATACAATGTG | | |
| | GATGTCGTTGGAACACTTCCTCTAGGGCTGCTACCTCCAGCCAATCCGGACACCAGCCTCTTCCACC | | |
| | TTGTGTACGTAGATGCCATTGCCATAGCCATCGTTGGATTTTCAGTGACCATCTCCATGGCCAAGAC | | |
| | CTTAGCAAATAAACATGGCTACCAGGTTGACGGCAATCAGGAGCTCATTGCCCTGGGACTGTGCAAT | | |
| | TCCATTGGCTCACTCTTCCAGACCTTTTCAATTTCATGCTCCTTGTCTCGAAGCCTTGTTCAGGAGG | | |
| | GAACCGGTGGGAAGACACAGGCTGTGCTGTCGGCCATTGTGATTGTCAACCTGAAGGGAATGTTTAT | | |
| | GCAGTTCTCAGATCTCCCCTTTTTCTGGAGAACCAGCAAAATAGAGCTGACCATCTGGCTTACCACT | | |
| | TTTGTGTCCTCCTTGTTCCTGGGATTGGACTATGGTTTGATCACTGCTGTGATCATTGCTCTGCTGA | | |
| | CTGTGATTTACAGAACACAGAGTCCAAGCTACAAAGTCCTTGGAAAGCTTCCTGAAACTGATGTGTA | | |
| | TATTGATATAGACGCATATGAGGAGGTGAAAGAAATTCCTGGAATAAAAATATTTCAAATAAATGCA | | |
| | CCAATTTACTATGCAAATAGCGACTTGTATAGCAATGCATTAAAACGAAAGACTGGAGTGAACCCAG | | |
| | CAGTCATCATGGGAGCAAGGAGAAAGGCCATGCGGAAGTACGCTAAGGAAGTCGGAAATGCAAATAT | | |
| | GGCCAACGCAACTGTTGTCAAAGCAGATGCAGAAGTAGATGGAGAGGATGCTACCAAGCCTGAAGAA | | |
| | GAGGATGGTGAAGTAAAATATCCCCCAATAGTGATCAAAAGCACATTTCCTGAGGAAATGCAAAGAT | | |
| | TTATGCCCCCAGGGGATAACGTCCACACTGTCATTTTGGATTTCACTCAAGTCAATTTTATTGATTC | | |
| | TGTTGGAGTGAAAACTCTGGCAGGGATTGTAAAAGAATATGGAGACGTCGGTATATATGTATACTTA | | |
| | GCAGGATGCAGTGCACAAGTTGTGAATGACCTCACTCGGAATAGATTTTTTGAAAATCCTGCCCTAT | | |
| | GGGAGCTGCTGTTCCACAGCATTCATGATGCAGTTTTAGGCAGCCAACTTAGAGAGGCACTTGCTGA | | |
| | ACAGGAAGCCTCGGCTCCCCCTTCCCAGGAGGACTTGGAGCCCAATGCCACTCCTGCCACTCCTGAG | | |
| | GCATAGATGAGGACCTCACCCTAGGATGGGGTTATAAGCCTCTCATGAAGTTCATAATTTACA | | |
| | ORF Start: ATG at 61 | | ORF Stop: TAG at 2215 |
| | SEQ ID NO: 88 | 718 aa | MW at 78546.4 kD |

| | |
|---|---|
| NOV 16a, | MDHAEENEILAATQRYYVERPIFSHPVLQERLHTKDKVPDSIADKLKQAFTCTPKKIRNIIYMFLPI |
| CG145494-01 | |
| Protein Sequence | TKWLPAYKFKEYVLGDLVSGISTGVLQLPQGLAFAMLAAVPPIFGLYSSFYPVIMYCFLGTSRHISI |
| | GPFAVISLMIGGVAVRLVPDDIVIPGGVNATNGTEARDALRVKVAMSVTLLSGIIQFCLGVCRFGFV |
| | AIYLTEPLVRGFTTAAAVHVFTSMLKYLFGVKTKRYSGIFSVVYSTVAVLQNVKNLNVCSLGVGLMV |
| | FGLLLGGKEFNERFKEKLPAPIPLEFFAVVMGTGISAGFNLKESYNVDVVGTLPLGLLPPANPDTSL |
| | FHLVYVDAIAIAIVGFSVTISMAKTLANKHGYQVDGNQELIALGLCNSIGSLFQTFSISCSLSRSLV |
| | QEGTGGKTQAVLSAIVIVNLKGMFMQFSDLPFFWRTSKIELTIWLTTFVSSLFLGLDYGLITAVIIA |
| | LLTVIYRTQSPSYKVLGKLPETDVYIDIDAYEEVKEIPGIKIFQINAPIYYANSDLYSNALKRKTGV |
| | NPAVIMGARRKAMRKYAKEVGNANMANATVVKADAEVDGEDATKPEEEDGEVKYPPIVIKSTFPEEM |
| | QRFMPPGDNVHTVILDFTQVNFIDSVGVKTLAGIVKEYGDVGIYVYLAGCSAQVVNDLTRNRFFENP |
| | ALWELLFHSIHDAVLGSQLREALAEQEASAPPSQEDLEPNATPATPEA |

Further analysis of the NOV16a protein yielded the following properties shown in Table 16B.

TABLE 16B
Protein Sequence Properties NOV16a

| | |
|---|---|
| PSort analysis: | 0.6000 probability located in plasma membrane; 0.4000 probability located in |
| | Golgi body; 0.3200 probability located in nucleus; 0.3000 probability located |
| | in endoplasmic reticulum (membrane) |
| SignalP analysis: | No Known Signal Sequence Predicted |

A search of the NOV16a protein against the Geneseq database, a proprietary database that contains sequences published in patents and patent publication, yielded several homologous proteins shown in Table 16C.

TABLE 16C
Geneseq Results for NOV16a

| | | NOV16a | | |
|---|---|---|---|---|
| Residues/ | Identities/ | | | |
| Geneseq | Protein/Organism/Length | Match | Similarities for | Expect |
| Identifier | [Patent #, Date] | Residues | the Matched Region | Value |
| AAY71067 | Human membrane transport | 9 . . . 684 | 291/741 (39%) | e−148 |
| | protein, MTRP-12 - Homo | 15 . . . 738 | 433/741 (58%) | |
| | sapiens, 758 aa. | | | |
| | [WO200026245-A2, | | | |
| | 11-MAY-2000] | | | |
| AAG67162 | Amino acid sequence of a | 9 . . . 684 | 289/734 (39%) | e−147 |
| | human 32613 transporter | 15 . . . 731 | 432/734 (58%) | |
| | polypeptide - Homo sapiens, | | | |
| | 751 aa. [WO200164875-A2, | | | |
| | 07-SEP-2001] | | | |
| ABG61914 | Prostate cancer-associated | 16 . . . 699 | 268/723 (37%) | e−144 |
| | protein #115 - Mammalia, | 20 . . . 741 | 419/723 (57%) | |
| | 790 aa. [WO200230268-A2, | | | |
| | 18-APR-2002] | | | |
| AAM51696 | Human pendrin SEQ ID NO | 16 . . . 699 | 268/723 (37%) | e−144 |
| | 2 - Homo sapiens, 780 aa. | 20 . . . 741 | 419/723 (57%) | |
| | [JP2001228146-A, | | | |
| | 24-AUG-2001] | | | |
| AAM51695 | Mouse pendrin SEQ ID | 16 . . . 688 | 270/713 (37%) | e−142 |
| | NO 1 - Mus sp, 780 aa. | 20 . . . 730 | 414/713 (57%) | |
| | [JP2001228146-A, | | | |
| | 24-AUG-2001] | | | |

In a BLAST search of public sequence datbases, the NOV16a protein was found to have homology to the proteins shown in the BLASTP data in Table 16D.

TABLE 16D
Public BLASTP Results for NOV16a

| | | NOV16a | | |
|---|---|---|---|---|
| Protein | | Residues/ | Identities/ | |
| Accession | | Match | Similarities for | Expect |
| Number | Protein/Organism/Length | Residues | the Matched Portion | Value |
| P58743 | Prestin - Homo sapiens | 1 . . . 718 | 718/744 (96%) | 0.0 |
| | (Human), 744 aa. | 1 . . . 744 | 718/744 (96%) | |
| Q9JKQ2 | Prestin - Meriones | 1 . . . 718 | 679/744 (91%) | 0.0 |
| | unguiculatus (Mongolian | 1 . . . 744 | 699/744 (93%) | |
| | jird) (Mongolian gerbil), 744 | | | |
| | aa. | | | |
| Q99NH7 | Prestin - Mus musculus | 1 . . . 718 | 680/744 (91%) | 0.0 |
| | (Mouse), 744 aa. | 1 . . . 744 | 700/744 (93%) | |
| Q9EPH0 | Prestin - Rattus norvegicus | 1 . . . 718 | 677/744 (90%) | 0.0 |
| | (Rat), 744 aa. | 1 . . . 744 | 699/744 (92%) | |
| AAH28856 | Solute carrier family 26, | 16 . . . 684 | 282/718 (39%) | e−148 |
| | member 6 - Mus musculus | 8 . . . 715 | 432/718 (59%) | |
| | (Mouse), 735 aa. | | | |

PFam analysis predicts that the NOV16a protein contains the domains shown in the Table 16E.

TABLE 16E
Domain Analysis of NOV16a

| | | Identities/ | |
|---|---|---|---|
| | | Similarities | |
| | | for the | Expect |
| Pfam Domain | NOV16a Match Region | Matched Region | Value |
| COX3 | 334 . . . 458 | 31/266 (12%) | 0.7 |
| | | 80/266 (30%) | |
| Sulfate_transp | 191 . . . 477 | 111/328 (34%) | 7e−78 |
| | | 234/328 (71%) | |
| STAS | 500 . . . 683 | 34/188 (18%) | 1.4e−12 |
| | | 124/188 (66%) | |

### Example 17

The NOV17 clone was analyzed, and the nucleotide and encoded polypeptide sequences are shown in Table 17A.

TABLE 17A
NOV17 Sequence Analysis

| | SEQ ID NO: 89 | 2124 bp | |
|---|---|---|---|
| NOV 17a, | AAGCTGAGGTCTTATAGATTGGTGGTACTTAAGGCAGAAAATTAACACCGTGTTTTGTAGCTGTTAG | | |
| CG145722-01 | | | |
| DNA Sequence | TTGGTAGAGGGAAATTCAGGCTACCGTCGCGAAACCTGCAGGTTAAGTTATTTTCTCCTCCCTGCTT | | |
| | CTGTAGGTTCACAGCGTTCCCTTCTGATAGAGCTTTTTGTCTGTGTTGTAAAGCTCTTTGGCTGAGA | | |
| | TGGATGACAAAGATATTGACAAAGAACTAAGGCAGAAATTAAACTTTTCCTATTGTGAGGAGACTGA | | |
| | GATTGAAGGGCAGAAGAAAGTAGAAGAAAGCAGGGAGGCTTCGAGCCAAACCCCAGAGAAGGGTGAA | | |
| | GTGCAGGATTCAGAGGCAAAGGGTACACCACCTTGGACTCCCCTTAGCAACGTGCATGAGCTCGACA | | |
| | CATCTTCGGAAAAAGACAAAGAAAGTCCAGATCAGATTTTGAGGACTCCAGTGTCACACCCTCTCAA | | |
| | ATGTCCTGAGACACCAGCCCAACCAGACAGCAGGAGCAAGCTGCTGCCCAGTGACAGCCCCTCTACT | | |
| | CCCAAAACCATGCTGAGCCGGTTGGTGATTTCTCCAACAGGGAAGCTTCCTTCCAGAGGCCCTAAGC | | |
| | ATTTGAAGCTCACACCTGCTCCCCTCAAGGATGAGATGACCTCATTGGCTCTGGTCAATATTAATCC | | |
| | CTTCACTCCAGAGTCCTATAAAAAATTATTTCTTCAATCTGGTGGCAAGAGGAAAATAAGAAGATGT | | |
| | GTTTTACGAGAAACCAACATGGCTTCCCGCTATGAAAAAGAATTCTTGGAGGTTGAAAAAATTGGGG | | |
| | TTGGCGAATTTGGTACAGTCTACAAGTGCATTAAGAGGCTGGATGGATGTGTTTATGCAATAAAGCG | | |
| | CTCTATGAAAACTTTTACAGAATTATCAAATGAGAATTCGGCTTTGCATGAAGTTTATGCTCACGCA | | |
| | GTGCTTGGGCATCACCCCCATGTGGTACGTTACTATTCCTCATGGGCAGAAGATGACCACATGATCA | | |
| | TTCAGAATGAATACTGCAATGGTGGGAGTTTGCAAGCTGCTATATCTGAAAACACTAAGTCTGGCAA | | |
| | TCATTTTGAAGAGCCAAAACTCAAGGACATCCTTCTACAGATTTCCCTTGGCCTTAATTACATCCAC | | |
| | AACTCTAGCATGGTACACCTGGACATCAAACCTAGTAATATATTCATTTGTCACAAGATGCAAAGTG | | |
| | AATCCTCTGGAGTCATAGAAGAAGTTGAAAATGAAGCTGATTGGTTTCTCTCTGCCAATGTGATGTA | | |
| | TAAAATTGGTGACCTGGGCCACGCAACATCAATAAACAAACCCAAAGTGGAAGAAGGAGATAGTCGC | | |
| | TTCCTGGCTAATGAGATTTTGCAAGAGGATTACCGGCACCTTCCCAAAGCAGACATATTTGCCTTGG | | |
| | GATTAACAATTGCAGTGGCTGCAGGAGCAGAGTCATTGCCCACCAATGGTGCTGCATGGCACCATAT | | |
| | CCGCAAGGGTAACTTTCCGGACGTTCCTCAGGAGCTCTCAGAAAGCTTTTCCAGTCTGCTCAAGAAC | | |
| | ATGATCCAACCTGATGCCGAACAGAGACCTTCTGCAGCAGCTCTGGCCAGAAATACAGTTCTCCGGC | | |
| | CTTCCCTGGGAAAAACAGAAGAGCTCCAACAGCAGCTGAATTTGGAAAAGTTCAAGACTGCCACACT | | |
| | GGAAAGGGAACTGAGAGAAGCCCAGCAGGCCCAGTCACCCCAGGGATATACCCATCATGGTGACACT | | |
| | GGGGTCTCTGGGACCCACACAGGATCAAGAAGCACAAAACGCCTGGTGGGAGGAAAGAGTGCAAGGT | | |
| | CTTCAAGCTTTACCTGTGAGTAATCTTCCCCTTAAGAACTCATTTTGCAGCCGGGCGTGGTGGCTCA | | |
| | CGCCTGTAATCCCAACACTTTGGGAGGCCAAGGCAGGTGGATCATGAGGTCAGGAGATCGAAACCAT | | |
| | CCTGGCTAACACGGTGAAACCCCATCTCTACTAAAAATACAAAAAATTAGCAGGGCGAGGTGGCAGG | | |
| | CGCCTATAATCCCAGCTACTCAGGAGGCTGAGGAAGGAGAATCGCTTGAACCCGGGAGGTGGAGCTT | | |
| | GCAGTGAGCTGAGATCACACCACTGCACTCCAGCCTGGGCAACAGAG | | |
| | ORF Start: ATG at 201 | | ORF Stop: TAA at 1830 |
| | SEQ ID NO: 90 | 543 aa | MW at 60514.5 kD |

| | |
|---|---|
| NOV 17a, | MDDKDIDKELRQKLNFSYCEETEIEGQKKVEESREASSQTPEKGEVQDSEAKGTPPWTPLSNVHELD |
| CG 145722-01 | |
| Protein Sequence | TSSEKDKESPDQILRTPVSHPLKCPETPAQPDSRSKLLPSDSPSTPKTMLSRLVISPTGKLPSRGPK |
| | HLKLTPAPLKDEMTSLALVNINPFTPESYKKLFLQSGGKRKIRRCVLRETNMASRYEKEFLEVEKIG |
| | VGEFGTVYKCIKRLDGCVYAIKRSMKTFTELSNENSALHEVYAHAVLGHHPHVVRYYSSWAEDDHMI |
| | IQNEYCNGGSLQAAISENTKSGNHFEEPKLKDILLQISLGLNYIHNSSMVHLDIKPSNIFICHKMQS |
| | ESSGVIEEVENEADWFLSANVMYKIGDLGHATSINKPKVEEGDSRFLANEILQEDYRHLPKADIFAL |
| | GLTIAVAAGAESLPTNGAAWHHIRKGNFPDVPQELSESFSSLLKNMIQPDAEQRPSAAALARNTVLR |
| | PSLGKTEELQQQLNLEKFKTATLERELREAQQAQSPQGYTHHGDTGVSGTHTGSRSTKRLVGGKSAR |
| | SSSFTCE |

Further analysis of the NOV17a protein yielded the following properties shown in Table 17B.

TABLE 17B
Protein Sequence Properties NOV17a

| | |
|---|---|
| PSort analysis: | 0.4500 probability located in cytoplasm; 0.3000 probability located in |
| | microbody (peroxisome); 0.1000 probability located in mitochondrial matrix |
| | space; 0.1000 probability located in lysosome (lumen) |
| SignalP analysis: | No Known Signal Sequence Predicted |

A search of the NOV17a protein against the Geneseq database, a proprietary database that contains sequences published in patents and patent publication, yielded several homologous proteins shown in Table 17C.

TABLE 17C
Geneseq Results for NOV17a

| Geneseq | Protein/Organism/Length | NOV 17a Residues/ | Identities/Similarities | Expect |
|---|---|---|---|---|
| Identifier | [Patent #, Date] | Match Residues | for the Matched Region | Value |
| AAB62519 | Xenopus Wee1 protein | 188 . . . 431 | 170/244 (69%) | 1e−94 |
| | catalytic domain (residues | 1 . . . 240 | 191/244 (77%) | |
| | 210-443) - Xenopus sp, 240 | | | |
| | aa. [US6225101-B1, | | | |
| | 01-MAY-2001] | | | |
| AAY51401 | Xenopus sp. Wee1 catalytic | 188 . . . 431 | 170/244 (69%) | 1e−94 |
| | domain protein fragment - | 1 . . . 240 | 191/244 (77%) | |
| | Xenopus sp, 240 aa. | | | |
| | [US6020194-A, | | | |
| | 01-FEB-2000] | | | |
| ABB60693 | Drosophila melanogaster | 109 . . . 501 | 180/464 (38%) | 9e−78 |
| | polypeptide SEQ ID NO | 101 . . . 551 | 257/464 (54%) | |
| | 8871 - Drosophila | | | |
| | melanogaster, 609 aa. | | | |
| | [WO200171042-A2, | | | |
| | 27-SEP-2001] | | | |
| AAY96776 | Z. mays partial wee1 kinase - | 185 . . . 465 | 103/282 (36%) | 3e−45 |
| | Zea mays, 525 aa. | 264 . . . 513 | 153/282 (53%) | |
| | [WO200037645-A2, | | | |
| | 29-JUN-2000] | | | |
| AAY96770 | Z. mays partial wee1 kinase - | 185 . . . 465 | 103/282 (36%) | 3e−45 |
| | Zea mays, 403 aa. | 142 . . . 391 | 153/282 (53%) | |
| | [WO200037645-A2, | | | |
| | 29-JUN-2000] | | | |

In a BLAST search of public sequence datbases, the NOV 17a protein was found to have homology to the proteins shown in the BLASTP data in Table 17D.

TABLE 17D
Public BLASTP Results for NOV17a

| Protein | | | | |
|---|---|---|---|---|
| Accession | | NOV17a Residues/ | Identities/Similarities | Expect |
| Number | Protein/Organism/Length | Match Residues | for the Matched Portion | Value |
| O95017 | WUGSC:H_DJ0894A10.2 | 1 . . . 541 | 541/541 (100%) | 0.0 |
| | protein - Homo sapiens | 1 . . . 541 | 541/541 (100%) | |
| | (Human), 541 aa (fragment). | | | |
| P47817 | Wee1-like protein kinase (EC | 10 . . . 542 | 291/560 (51%) | e−143 |
| | 2.7.1.112) - Xenopus laevis | 11 . . . 552 | 352/560 (61%) | |
| | (African clawed frog), 555 | | | |
| | aa. | | | |
| O57473 | Wee1 homolog - Xenopus | 10 . . . 542 | 294/566 (51%) | e−143 |
| | laevis (African clawed frog), | 11 . . . 551 | 357/566 (62%) | |
| | 554aa. | | | |
| Q8QGV2 | Wee1B kinase - Xenopus | 10 . . . 541 | 263/579 (45%) | e−122 |
| | laevis (African clawed frog), | 20 . . . 593 | 350/579 (60%) | |
| | 595 aa. | | | |
| Q63802 | Wee1-like protein kinase (EC | 92 . . . 541 | 236/484 (48%) | e−118 |
| | 2.7.1.112) -Rattus | 168 . . . 644 | 308/484 (62%) | |
| | norvegicus (Rat), 646 aa. | | | |

PFam analysis predicts that the NOV 17a protein contains the domains shown in the Table 17E.

TABLE 17E
Domain Analysis of NOV17a

| | NOV17a | Identities/Similarities | Expect |
|---|---|---|---|
| Pfam Domain | Match Region | for the Matched Region | Value |
| pkinase | 194 . . . 462 | 73/310 (24%) | 6.4e−45 |
| | | 193/310 (62%) | |

### Example 18

The NOV18 clone was analyzed, and the nucleotide and encoded polypeptide sequences are shown in Table 18A.

TABLE 18A
NOV18 Sequence Analysis

| | SEQ ID NO 91 | 753 bp | |
|---|---|---|---|
| NOV18a, | TCCCTTCTCCTGCCCCTGCAGATCCTACTGCTATCCTTAGCCTTGGAAACTGCAGGAGAAGAAGCCC | | |
| CG145754-01 | | | |
| DNA Sequence | AGGGTGACAAGATTATTGATGGCGCCCCATGTGCAAGAGGCTCCCACCCATGGCAGGTGGCCCTGCT | | |
| | CAGTGGCAATCAGCTCCACTGCGGAGGCGTCCTGGTCAATGAGCGCTGGGTGCTCACTGCCGCCCAC | | |
| | TGCAAGATGAATGAGTACACCGTGCACCTGGGCAGTGATACGCTGGGCGACAGGAGAGCTCAGAGGA | | |
| | TCAAGGCCTCGAAGTCATTCCGCCACCCCGGCTACTCCACACAGACCCATGTTAATGACCTCATGCT | | |
| | CGTGAAGCTCAATAGCCAGGCCAGGCTGTCATCCATGGTGAAGAAAGTCAGGCTGCCCTCCCGCTGC | | |
| | GAACCCCCTGGAACCACCTGTACTGTCTCCGGCTGGGGCACTACCACGAGCCCAGATGTGACCTTTC | | |
| | CCTCTGACCTCATGTGCGTGGATGTCAAGCTCATCTCCCCCCAGGACTGCACGAAGGTTTACAAGGA | | |
| | CTTACTGGAAAATTCCATGCTGTGCGCTGGCATCCCCGACTCCAAGAAAAACGCCTGCAATGGTGAC | | |
| | TCAGGGGGACCGTTGGTGTGCAGAGGTACCCTGCAAGGTCTGGTGTCCTGGGGAACTTTCCCTTGCG | | |
| | GCCAACCCAATGACCCAGGAGTCTACACTCAAGTGTGCAAGTTCACCAAGTGGATAAATGACACCAT | | |
| | GAAAAAGCATCGCTAA | | |
| | ORF Start: at 1 | | ORF Stop: TAA at 751 |
| | SEQ ID NO: 92 | 250 aa | MW at 27166.0 kD |
| NOV18a, | SLLLPLQILLLSLALETAGEEAQGDKIIDGAPCARGSHPWQVALLSGNQLHCGGVLVNERWVLTAAH | | |
| CG145754-01 | | | |
| Protein Sequence | CKMNEYTVHLGSDTLGDRRAQRIKASKSFRHPGYSTQTHVNDLMLVKLNSQARLSSMVKKVRLPSRC | | |
| | EPPGTTCTVSGWGTTTSPDVTFPSDLMCVDVKLISPODCTKVYKDLLENSMLCAGIPDSKKNACNGD | | |
| | SGGPLVCRGTLQGLVSWGTFPCGQPNDPGVYTQVCKFTKWINDTMKKHR | | |
| | SEQ ID NO:93 | 862 bp | |
| NOV18b, | ACTGGGTCCGAATCAGTAGGTGACCCCGCCCCTGGATTCTGGAAGACCTCACCATGGGACGCCCCCG | | |
| CG145754-03 | | | |
| DNA Sequence | ACCTCGTGCGGCCAAGACGTGGATGTTCCTGCTCTTACTGGGGGGAGCCTGGGCAGCCAGGGGTGAC | | |
| | AAGATTATTGATGGCGCCCCATGTGCAAGAGGCTCCCACCCATGGCAGGTGGCCCTGCTCAGTGGCA | | |
| | ATCAGCTCCACTGCGGAGGCGTCCTGGTCAATGAGCGCTGGGTGCTCACTGCCGCCCACTGCAAGAT | | |
| | GAATGAGTACACCGTGCACCTGGGCAGTGATACGCTGGGCGACAGGAGAGCTCAGAGGATCAAGGCC | | |
| | TCGAAGTCATTCCGCCACCCCGGCTACTCCACACAGACCCATGTTAATGACCTCATGCTCGTGAAGC | | |
| | TCAATAGCCAGGCCAGGCTGTCATCCATGGTGAAGAAAGTCAGGCTGCCCTCCCGCTGCGAACCCCC | | |
| | TGGAACCACCTGTACTGTCTCCGGCTGGGGCACTACCACGAGCCCAGATGTGACCTTTCCCTCTGAC | | |
| | CTCATGTGCGTGGATGTCAAGCTCATCTCCCCCCAGGACTGCACGAAGGTTTACAAGGACTTACTGG | | |
| | AAAATTCCATGCTGTGCGCTGGCATCCCCGACTCCAAGAAAAACGCCTGCAATGGTGACTCAGGGGG | | |
| | ACCGTTGGTGTGCAGAGGTACCCTGCAAGGTCTGGTGTCCTGGGGAACTTTCCCTTGCGGCCAACCC | | |
| | AATGACCCAGGAGTCTACACTCAAGTGTGCAAGTTCACCAAGTGGATAAATGACACCATGAAAAAGC | | |
| | ATCGCTAACGCCACACTGAGTTAATTAACTGTGTGCTTCCAACAGAAAATGCACAGGA | | |
| | ORF Start: ATG at 54 | | ORF Stop: TAA at 810 |
| | SEQ ID NO: 94 | 252 aa | MW at 27557.6 kD |
| NOV18b, | MGRPRPRAAKTWMFLLLLGGAWAARGDKIIDGAPCARGSHPWQVALLSGNQLHCGGVLVNERWVLTA | | |
| CG145754-03 | | | |
| Protein Sequence | AHCKMNEYTVHLGSDTLGDRRAQRIKASKSFRHPGYSTQTHVNDLMLVKLNSQARLSSMVKKVRLPS | | |
| | RCEPPGTTCTVSGWGTTTSPDVTFPSDLMCVDVKLISPQDCTKVYKDLLENSMLCAGIPDSKKNACN | | |
| | GDSGGPLVCRGTLQGLVSWGTFPCGQPNDPGVYTQVCKFTKWINDTMKKHR | | |
| | SEQ ID NO:95 | 804 bp | |
| NOV18c, | GGATTTCCGGGCTCCATGGCAAGATCCCTTCTCCTGCCCCTGCAGATCCTACTGCTATCCTTAGCCT | | |
| CG145754-02 | | | |
| DNA Sequence | TGGAAACTGCAGGAGAAGAAGCCCAGGGTGACAAGATTATTGATGGCGCCCCATGTGCAAGAGGCTC | | |
| | CCACCCATGGCAGGTGGCCCTGCTCAGTGGCAATCAGCTCCACTGCGGAGGCGTCCTGGTCAATGAG | | |
| | CGCTGGGTGCTCACTGCCGCCCACTGCAAGATGAATGAGTACACCGTGCACCTGGGCAGTGATACGC | | |
| | TGGGCGACAGGAGAGCTCAGAGGATCAAGGCCTCGAAGTCATTCCGCCACCCCGGCTACTCCACACA | | |
| | GACCCATGTTAATGACCTCAAGCTCATCTCCCCCCAGGACTGCACGAAGGTTTACAAGGACTTACTG | | |
| | GAAAATTCCATGCTGTGCGCTGGCATCCCCGACTCCAAGAAAAACGCCTGCAATGGTGACTCAGGGG | | |
| | GACCGTTGGTGTGCAGAGGTACCCTGCAAGGTCTGGTGTCCTGGGGAACTTTCCCTTGCGGCCAACC | | |
| | CAATGACCCAGGAGTCTACACTCAAGTGTGCAAGTTCACCAAGTGGATAAATGACACCATGAAAAAG | | |
| | CATCGCTAACGCCACACTGAGTTAATTAACTGTGTGCTTCCAACAGAAATGCACAGGAGTGAGGAC | | |
| | GCCGATGACCTATGAAGTCAAATTTGACTTTACCTTTCCTCAAAGATATATTTAAACCTCATGCCCT | | |
| | GTTGATAAACCAATCAAATTGGTAAAGACCTAAAACCAAAACAAATAAAGAAACACAAAACCCTCAA | | |
| | ORF Start: ATG at 16 | | ORF Stop: TAA at 610 |
| | SEQ ID NO:96 | 198 aa | MW at 21613.6 kD |

| NOV18c, | MARSLLLPLQILLLSLALETAGEEAQGDKIIDGAPCARGSHPWQVALLSGNQLHCGGVLVNERWVLT | | |
|---|---|---|---|
| CG145754-02 | | | |
| Protein Sequence | AAHCKMNEYTVHLGSDTLGDRRAQRIKASKSFRHPGYSTQTHVNDLKLISPQDCTKVYKDLLENSML | | |
| | CAGIPDSKKNACNGDSGGPLVCRGTLQGLVSWGTFPCGQPNDPGVYTQVCKFTKWINDTMKKHR | | |
| | SEQ ID NO:97 | 544 bp | |
| NOV18d, | CACCGGATCCGAAGAAGCCCAGGGTGACAAGATTATTGATGGCGCCCCATGTGCAAGAGGCTCCCAC | | |
| 252718128 DNA | | | |
| Sequence | CCATGGCAGGTGGCCCTGCTCAGTGGCAATCAGCTCCACTGCGGAGGCGTCCTGGTCAATGAGCGCT | | |
| | GGGTGCTCACTGCCGCCCACTGCAAGATGAATGAGTACACCGTGCACCTGGGCAGTGATACGCTGGG | | |
| | CGACAGGAGAGCTCAGAGGATCAAGGCCTCGAAGTCATTCCGCCACCCCGGCTACTCCACACAGACC | | |
| | CATGTTAATGACCTCAAGCTCATCTCCCCCCAGGACTGCACGAAGGTTTACAAGGACTTACTGGAAA | | |
| | ATTCCATGCTGTGCGCTGGCATCCCCGACTCCAAGAAAAACGCCTGCAATGGTGACTCAGGGGGACC | | |
| | GTTGGTGTGCAGAGGTACCCTGCAAGGTCTGGTGTCCTGGGGAACTTTCCCTTGCGGCCAACCCAAT | | |
| | GACCCAGGAGTCTACACTCAAGTGTGCAAGTTCACCAAGTGGATAAATGACACCATGAAAAAGCATC | | |
| | TCGAGGGC | | |
| | ORF Start: at 2 | | ORF Stop: end of sequence |
| | SEQ ID NO:98 | 181 aa | MW at 19683.2 kD |
| NOV18d, | TGSEEAQGDKIIDGAPCARGSHPWQVALLSGNQLHCGGVLVNERWVLTAAHCKMNEYTVHLGSDTLG | | |
| 252718128 | | | |
| Protein Sequence | DRRAQRIKASKSFRHPGYSTQTHVNDLKLISPQDCTKVYKDLLENSMLCAGIPDSKKNACNGDSGGP | | |
| | LVCRGTLQGLVSWGTFPCGQPNDPGVYTQVCKFTKWINDTMKKHLEG | | |
| | SEQ ID NO: 99 | 292 bp | |
| NOV18e, | CACCGGATCCGAAGAAGCCCAGGGTGACAAGATTATTGATGGCGCCCCATGTGCAAGAGGCTCCCAC | | |
| 252718152 DNA | | | |
| Sequence | CCATGGCAGGTGGCCCTGCTCAGTGGCAATCAGCTCCACTGCGGAGGCGTCCTGGTCAATGAGCGCT | | |
| | GGGTGCTCACTGCCGCCCACTGCAAGATGAATGAGTACACCGTGCACCTGGGCAGTGATACGCTGGG | | |
| | CGACAGGAGAGCTCAGAGGATCAAGGCCTCGAAGTCATTCCGCCACCCCGGCTACTCCACACAGACC | | |
| | CATGTTAATGACCTCCTCGAGGGC | | |
| | ORF Start: at 2 | | ORF Stop: end of sequence |
| | SEQ ID NO: 100 | 97 aa | MW at 10551.7 kD |
| NOV18e, | TGSEEAQGDKIIDGAPCARGSHPWQVALLSGNQLHCGGVLVNERWVLTAAHCKMNEYTVHLGSDTLG | | |
| 252718152 | | | |
| Protein Sequence | DRRAQRIKASKSFRHPGYSTQTHVNDLLEG | | |
| | SEQ ID NO: 101 | 742 bp | |
| NOV18f, | AGGCTCCGCGGCCGCCCCCTTCACCGGATCCGCCAGGGGTGACAAGATTATTGATGGCGCCCCATGT | | |
| 247856668 DNA | | | |
| Sequence | GCAAGAGGCTCCCACCCATGGCAGGTGGCCCTGCTCAGTGGCAATCAGCTCCACTGCGGAGGCGTCC | | |
| | TGGTCAATGAGCGCTGGGTGCTCACTGCCGCCCACTGCAAGATGAATGAGTACACCGTGCACCTGGG | | |
| | CAGTGATACGCTGGGCGACAGGAGAGCTCAGAGGATCAAGGCCTCGAAGTCATTCCGCCACCCCGGC | | |
| | TACTCCACACAGACCCATGTTAATGACCTCATGCTCGTGAAGCTCAATAGCCAGGCCAGGCTGTCAT | | |
| | CCATGGTGAAGAAAGTCAGGCTGCCCTCCCGCTGCGAACCCCCTGGAACCACCTGTACTGTCTCCGG | | |
| | CTGGGGCACTACCACGAGCCCAGATGTGACCTTTCCCTCTGACCTCATGTGCGTGGATGTCAAGCTC | | |
| | ATCTCCCCCCAGGACTGCACGAAGGTTTACAAGGACTTACTGGAAAATTCCATGCTGTGCGCTGGCA | | |
| | TCCCCGACTCCAAGAAAAACGCCTGCAATGGTGACTCAGGGGGACCGTTGGTGTGCAGAGGTACCCT | | |
| | GCAAGGTCTGGTGTCCTGGGGAACTTTCCCTTGCGGCCAACCCAATGACCCAGGAGTCTACACTCAA | | |
| | GTGTGCAAGTTCACCAAGTGGATAAATGACACCATGAAAAAGCATCGCCTCGAGGGCAAGGGTGGGC | | |
| | GCGCC | | |
| | ORF Start: at 2 | | ORF Stop: end of sequence |
| | SEQ ID NO: 102 | 247 aa | MW at 26591.2 kD |
| NOV18f, | GSAAAPFTGSARGDKIIDGAPCARGSHPWQVALLSGNQLHCGGVLVNERWVLTAAHCKMNEYTVHLG | | |
| 247856668 | | | |
| Protein Sequence | SDTLGDRRAQRIKASKSFRHPGYSTQTHVNDLMLVKLNSQARLSSMVKKVRLPSRCEPPGTTCTVSG | | |
| | WGTTTSPDVTFPSDLMCVDVKLISPQDCTKVYKDLLENSMLCAGIPDSKKNACNGDSGGPLVCRGTL | | |
| | QGLVSWGTFPCGQPNDPGVYTQVCKFTKWINDTMKKHRLEGKGGRA | | |
| | SEQ ID NO: 103 | 673 bp | |
| NOV18g, | AGGCTCCGCGGCCGCCCCCTTCACCGGATCCGCCAGGGGTGACAAGATTATTGATGGCGCCCCATGT | | |
| 247856705 DNA | | | |
| Sequence | GCAAGAGGCTCCCACCCATGGCAGGTGGCCCTGCTCAGTGGCAATCAGCTCCACTGCGGAGGCGTCC | | |
| | TGGTCAATGAGCGCTGGGTGCTCACTGCCGCCCACTGCAAGATGAATGAGTACACCGTGCACCTGGG | | |
| | CAGTGATACGCTGGGCGACAGGAGAGCTCAGAGGATCAAGGCCTCGAAGTCATTCCGCCACCCCGGC | | |
| | TACTCCACACAGACCCATGTTAATGACCTCATGCTCGTGAAGCTCAATAGCCAGGCCAGGCTGTCAT | | |
| | CCATGGTGAAGAAAGTCAGGCTGCCCTCCCGCTGCGAACCCCCTGGAACCACCTGTACTGTCTCCGG | | |
| | CTGGGGCACTACCACGAGCCCAGATGTGACCTTTCCCTCTGACCTCATGTGCGTGGATGTCAAGCTC | | |
| | ATCTCCCCCCAGGACTGCACGAAGGTTTACAAGGACTTACTGGAAAATTCCATGCTGTGCGCTGGCA | | |
| | TCCCCGACTCCAAGAAAAACGCCTGCAATGGTGACTCAGGGGGACCGTTGGTGTGCAGAGGTACCCT | | |
| | GCAAGGTCTGGTGTCCTGGGGAACTTTCCCTTGCGGCCAACCCAATCTCGAGGGCAAGGGTGGGCGC | | |
| | GCC | | |
| | ORF Start: at 2 | | ORF Stop: end of sequence |
| | SEQ ID NO: 104 | 224 aa | MW at 23813.0 kD |

| | |
|---|---|
| NOV18g, | GSAAAPFTGSARGDKIIDGAPCARGSHPWQVALLSGNQLHCGGVLVNERWVLTAAHCKMNEYTVHLG |
| 247856705 | |
| Protein Sequence | SDTLGDRRAQRIKASKSFRHPGYSTQTHVNDLMLVkLNSQARLSSMVKKVRLPSRCEPPGTTCTVSG |
| | WGTTTSPDVTFPSDLMCVDVKLISPQDCTKVYKDLLENSMLCAGIPDSKKNACNGDSGGPLVCRGTL |
| | QGLVSWGTFPCGQPNLEGKGGRA |

Sequence comparison of the above protein sequences yields the following sequence relationships shown in Table 18B.

TABLE 18B
Comparison of NOV18a against NOV18b through NOV18g.

| | NOV18a Residues/ | Identities/Similarities |
|---|---|---|
| Protein Sequence | Match Residues | for the Matched Region |
| NOV18b | 25 . . . 250 | 213/226 (94%) |
| | 27 . . . 252 | 213/226 (94%) |
| NOV18c | 16 . . . 250 | 176/235 (74%) |
| | 19 . . . 198 | 177/235 (74%) |
| NOV18d | 17 . . . 249 | 172/233 (73%) |
| | 1 . . . 178 | 173/233 (73%) |
| NOV18e | 17 . . . 111 | 92/95 (96%) |
| | 1 . . . 95 | 93/95 (97%) |
| NOV18f | 22 . . . 250 | 215/229 (93%) |
| | 11 . . . 239 | 216/229 (93%) |
| NOV18g | 22 . . . 230 | 193/209 (92%) |
| | 11 . . . 219 | 194/209 (92%) |

Further analysis of the NOV18a protein yielded the following properties shown in Table 18C.

TABLE 18C
Protein Sequence Properties NOV18a

| | |
|---|---|
| PSort | 0.6233 probability located in outside; 0.1000 probability |
| analysis: | located in endoplasmic reticulum (membrane); 0.1000 |
| | probability located in endoplasmic reticulum (lumen); 0.1000 |
| | probability located in microbody (peroxisome) |
| SignalP | Cleavage site between residues 20 and 21 |
| analysis: | |

A search of the NOV18a protein against the Geneseq database, a proprietary database that contains sequences published in patents and patent publication, yielded several homologous proteins shown in Table 18D.

TABLE 18D
Geneseq Results for NOV18a

| Geneseq | Protein/Organism/Length | NOV18a Residues/ | Identities/Similarities | Expect |
|---|---|---|---|---|
| Identifier | [Patent #, Date] | Match Residues | for the Matched Region | Value |
| AAU82740 | Amino acid sequence of | 1 . . . 250 | 250/250 (100%) | e−150 |
| | novel human protease #39 - | 4 . . . 253 | 250/250 (100%) | |
| | Homo sapiens, 253 aa. | | | |
| | [WO200200860-A2, | | | |
| | 03-JAN-2002] | | | |
| AAW05383 | Human amyloid precursor | 1 . . . 250 | 250/250 (100%) | e−150 |
| | protein protease - Homo | 4 . . . 253 | 250/250 (100%) | |
| | sapiens, 253 aa. | | | |
| | [WO9631122-A1, | | | |
| | 10-OCT-1996] | | | |
| AAR67888 | Human stratum corneum | 1 . . . 250 | 250/250 (100%) | e−150 |
| | chymotrophic recombinant | 4 . . . 253 | 250/250 (100%) | |
| | enzyme (SCCE) - Homo | | | |
| | sapiens, 253 aa. | | | |
| | [WO9500651-A, | | | |
| | 05-JAN-1995] | | | |
| AAB21326 | Human HSCEE - Homo | 1 . . . 250 | 249/255 (97%) | e−146 |
| | sapiens, 257 aa. | 4 . . . 257 | 249/255 (97%) | |
| | [WO200053776-A2, | | | |
| | 14-SEP-2000] | | | |
| AAB98502 | Human Stratum Corneum | 26 . . . 250 | 225/225 (100%) | e−136 |
| | Chymotryptic Enzyme, | 1 . . . 225 | 225/225 (100%) | |
| | SCCE, catalytic domain - | | | |
| | Homo sapiens, 225 aa. | | | |
| | [WO200129056-A1 | | | |
| | 26-APR-2001] | | | |

In a BLAST search of public sequence datbases, the NOV18a protein was found to have homology to the proteins shown in the BLASTP data in Table 18E.

TABLE 18E
Public BLASTP Results for NOV18a

| Protein | | | | |
|---|---|---|---|---|
| Accession | | NOV18a Residues/ | Identities/Similarities | Expect |
| Number | Protein/Organism/Length | Match Residues | for the Matched Portion | Value |
| P49862 | Kallikrein 7 precursor (EC | 1 . . . 250 | 250/250 (100%) | e−149 |
| | 3.4.21.-) (Stratum corneum | 4 . . . 253 | 250/250 (100%) | |
| | chymotryptic enzyme) | | | |
| | (hSCCE) - Homo sapiens | | | |
| | (Human), 253 aa. | | | |
| AAH32005 | Kallikrein 7 (chymotryptic, | 1 . . . 250 | 249/250 (99%) | e−148 |
| | stratum corneum) - Homo | 4 . . . 253 | 249/250 (99%) | |
| | sapiens (Human), 253 aa. | | | |
| Q91VE3 | Thymopsin (Stratum | 3 . . . 250 | 185/248 (74%) | e−111 |
| | corneum chymotryptic | 5 . . . 249 | 212/248 (84%) | |
| | enzyme) - Mus musculus | | | |
| | (Mouse), 249 aa. | | | |
| AAN03663 | Kallikrein 7 short variant | 70 . . . 250 | 181/181 (100%) | e−107 |
| | protein - Homo sapiens | 1 . . . 181 | 181/181 (100%) | |
| | (Human), 181 aa. | | | |
| Q9R048 | Stratum corneum | 3 . . . 235 | 175/233 (75%) | e−102 |
| | chymotryptic enzyme - Mus | 5 . . . 234 | 198/233 (84%) | |
| | musculus (Mouse), 234 aa | | | |
| | (fragment). | | | |

PFam analysis predicts that the NOV18a protein contains the domains shown in the Table 18F.

TABLE 18F
Domain Analysis of NOV18a

| | NOV18a | Identities/Similarities | Expect |
|---|---|---|---|
| Pfam Domain | Match Region | for the Matched Region | Value |
| trypsin | 27 . . . 242 | 93/262 (35%) | 3.8e−87 |
| | | 182/262 (69%) | |

### Example 19

The NOV19 clone was analyzed, and the nucleotide and encoded polypeptide sequences are shown in Table 19A.

TABLE 19A
NOV19 Sequence Analysis

| | SEQ ID NO: 105 | 2028 bp | |
|---|---|---|---|
| NOV19a, | TTGAGGACTTTATTATTATTTGGGTTCTTTTCATTTCTTCCCCTTCTGGGCAACGAAGCAATGAAAT | | |
| CG146279-01 | | | |
| DNA Sequence | TTCCAATCGAGACGCCAAGAAAACAGGTGAACTGGGATCCTAAAGTGGCCGTTCCCGCAGCAGCACC | | |
| | GGTGTGCCAGCCCAAGAGCGCCACTAACGGGCAACCCCCGGCTCCGGCTCCGACTCCAACTCCGCGC | | |
| | CTGTCCATTTCCTCCCGAGCCACAGTGGTAGCCAGGATGGAAGGCACCTCCCAAGGGGGCTTGCAGA | | |
| | CCGTCATGAAGTGGAAGACGGTGGTTGCCATCTTTGTGGTTGTGGTGGTCTACCTTGTCACTGGCGG | | |
| | TCTTGTCTTCCGGGCATTGGAGCAGCCCTTTGAGAGCAGCCAGAAGAATACCATCGCCTTGGAGAAG | | |
| | GCGGAATTCCTGCGGGATCATGTCTGTGTGAGCCCCCAGGAGCTGGAGACGTTGATCCAGCATGCTC | | |
| | TTGATGCTGACAATGCGGGAGTCAGTCCAATAGGAAACTCTTCCAACAACAGCAGCCACTGGGACCT | | |
| | CGGCAGTGCCTTTTTCTTTGCTGGAACTGTCATTACGACCATAGGGTATGGGAATATTGCTCCGAGC | | |
| | ACTGAAGGAGGCAAAATCTTTTGTATTTTATATGCCATCTTTGGAATTCCACTCTTTGGTTTCTTAT | | |
| | TGGCTGGAATTGGAGACCAACTTGGAACCATCTTTGGGAAAAGCATTGCAAGAGTGGAGAAGGTCTT | | |
| | TCGAAAAAAGCAAGTGAGTCAGACCAAGATCCGGGTCATCTCAACCATCCTGTTCATCTTGGCCGGC | | |
| | TGCATTGTGTTTGTGACGATCCCTGCTGTCATCTTTAAGTACATCGAGGGCTGGACGGCCTTGGAGT | | |
| | CCATTTACTTTGTGGTGGTCACTCTGACCACGGTGGGCTTTGGTGATTTTGTGGCAGGGGGAAACGC | | |
| | TGGCATCAATTATCGGGAGTGGTATAAGCCCCTAGTGTGGTTTTGGATCCTTGTTGGCCTTGCCTAC | | |
| | TTTGCAGCTGTCCTCAGTATGATCGGAGATTGGCTACGGGTTCTGTCCAAAAAGACAAAAGAAGAGG | | |
| | TGGGTGAAATCAAGGCCCATGCGGCAGAGTGGAAGGCCAATGTCACGGCTGAGTTCCGGGAGACACG | | |
| | GCGAAGGCTCAGCGTGGAGATCCACGATAAGCTGCAGCGGGCGGCCACCATCCGCAGCATGGAGCGC | | |
| | CGGCGGCTGGGCCTGGACCAGCGGGCCCACTCACTGGACATGCTGTCCCCCGAGAAGCGCTCTGTCT | | |
| | TTGCTGCCCTGGACACCGGCCGCTTCAAGGCCTCATCCCAGGAGAGCATCAACAACCGGCCCAACAA | | |
| | CCTGCGCCTGAAGGGGCCGGAGCAGCTGAACAAGCATGGGCAGGGTGCGTCCGAGGACAACATCATC | | |
| | AACAAGTTCGGGTCCACCTCCAGACTCACCAAGAGGAAAAACAAGGACCTCAAAAAGACCTTGCCCG | | |
| | AGGACGTTCAGAAAATCTACAAGACCTTCCGGAATTACTCCCTGGACGAGGAGAAGAAAGAGGAGGA | | |
| | GACGGAAAAGATGTGTAACTCAGACAACTCCAGCACAGCCATGCTGACGGACTGTATCCAGCAGCAC | | |
| | GCTGAGTTGGAGAACGGAATGATACCCACGGACACCAAAGACCGGGAGCCGGAGAACAACTCATTAC | | |
| | TTGAAGACAGAAACTAAATGTGAAGGACATTGGTCTTGGACTGAGCGTTGTGTGTGTGTGTGTGTGT | | |
| | GTTTTTAATATTCACACTGAGACATGTGCCTTAAACAGACTTTTTAGTCCAAAATTACATAGCATTG | | |
| | AAGAATATATTTCACTGTGCCATAAACAACTGAAAGCTTGCTCTGCCAAAAGGAATCAGAGAACAAG | | |
| | AACTTCATTTCAGATAGCAAACGCAGGACACACCAAGAGTGTCCGTGCACGTAGCCGGTTCTGGCCG | | |
| | TACATGTTAAGGGCATTTCAGTGGCAGTGCTGTACCCCTGGGCAGTGCTACCTGGGCACACACGTAG | | |
| | ACAAGGGCAGCTATTCCT | | |
| | ORF Start: ATG at 61 | | ORF Stop: TAA at 1690 |
| | SEQ ID NO: 106 | 543 aa | MW at 60334.6 kD |

| | |
|---|---|
| NOV19a, | MKFPIETPRKQVNWDPKVAVPAAAPVCQPKSATNGQPPAPAPTPTPRLSISSRATVVARMEGTSQGG |
| CG146279-01 | |
| Protein Sequence | LQTVMKWKTVVAIFVVVVVYLVTGGLVFRALEQPFESSQKNTIALEKAEFLRDHVCVSPQELETLIQ |
| | HALDADNAGVSPIGNSSNNSSHWDLGSAFFFAGTVITTIGYGNIAPSTEGGKIFCILYAIFGIPLFG |
| | FLLAGIGDQLGTIFGKSIARVEKVFRKKQVSQTKIRVISTILFILAGCIVFVTIPAVIFKYIEGWTA |
| | LESIYFVVVTLTTVGFGDFVAGGNAGINYREWYKPLVWFWILVGLAYFAAVLSMIGDWLRVLSKKTK |
| | EEVGEIKAHAAEWKANVTAEFRETRRRLSVEIHDKLQRAATIRSMERRRLGLDQRAHSLDMLSPEKR |
| | SVFAALDTGRFKASSQESINNRPNNLRLKGPEQLNKHGQGASEDNIINKFGSTSRLTKRKNKDLKKT |
| | LPEDVQKIYKTFRNYSLDEEKKEEETEKMCNSDNSSTAMLTDCIQQHAELENGMIPTDTKDREPENN |
| | SLLEDRN |

Further analysis of the NOVI9a protein yielded the following properties shown in Table 19B.

TABLE 19B
Protein Sequence Properties NOV19a

| | |
|---|---|
| PSort | 0.6000 probability located in plasma membrane; 0.4000 |
| analysis: | probability located in Golgi body; 0.3000 probability located |
| | in endoplasmic reticulum (membrane); 0.3000 probability |
| | located in microbody (peroxisome) |
| SignalP | No Known Signal Sequence Predicted |
| analysis: | |

A search of the NOV19a protein against the Geneseq database, a proprietary database that contains sequences published in patents and patent publication, yielded several homologous proteins shown in Table 19C.

TABLE 19C
Geneseq Results for NOV19a

| Geneseq | Protein/Organism/Length | NOV19a Residues/ | Identities/Similarities | Expect |
|---|---|---|---|---|
| Identifier | [Patent #, Date] | Match Residues | for the Matched Region | Value |
| AAU81354 | Novel human ion channel | 1 . . . 543 | 543/543 (100%) | 0.0 |
| | protein #34 - Homo sapiens, | 1 . . . 543 | 543/543 (100%) | |
| | 543 aa. [WO200185788-A2, | | | |
| | 15-NOV-2001] | | | |
| AAU79472 | Human novel transporter | 1 . . . 543 | 543/543 (100%) | 0.0 |
| | protein - Homo sapiens, 543 | 1 . . . 543 | 543/543 (100%) | |
| | aa. [WO200224748-A2, | | | |
| | 28-MAR-2002] | | | |
| AAU79473 | Human novel transporter | 1 . . . 543 | 542/543 (99%) | 0.0 |
| | protein variant - Homo | 1 . . . 543 | 543/543 (99%) | |
| | sapiens, 543 aa. | | | |
| | [WO200224748-A2, | | | |
| | | 28-MAR-2002] | | |
| AAE16596 | Human TWIK-Related K+ | 18 . . . 543 | 526/526 (100%) | 0.0 |
| | channel-2 (TREK-2) protein - | 13 . . . 538 | 526/526 (100%) | |
| | Homo sapiens, 538 aa. | | | |
| | [WO200200715-A2, | | | |
| | 03-JAN-2002] | | | |
| AAB47930 | Human TREK2 - Homo | 18 . . . 543 | 526/526 (100%) | 0.0 |
| | sapiens, 538 aa. | 13 . . . 538 | 526/526 (100%) | |
| | [WO200200715-A2, | | | |
| | 03-JAN-2002] | | | |

In a BLAST search of public sequence datbases, the NOV19a protein was found to have homology to the proteins shown in the BLASTP data in Table 19D.

TABLE 19D
Public BLASTP Results for NOV19a

| Protein | | | | |
|---|---|---|---|---|
| Accession | | NOV19a Residues/ | Identities/Similarities | Expect |
| Number | Protein/Organism/Length | Match Residues | for the Matched Portion | Value |
| Q8TDK7 | Potassium channel TREK2 | 1 . . . 543 | 542/543 (99%) | 0.0 |
| | splice variant b - Homo | 1 . . . 543 | 542/543 (99%) | |
| | sapiens (Human), 543 aa. | | | |
| P57789 | Potassium channel subfamily | 18 . . . 543 | 526/526 (100%) | 0.0 |
| | K member 10 (Outward | 13 . . . 538 | 526/526 (100%) | |
| | rectifying potassium channel | | | |
| | protein TREK-2) (TREK-2 | | | |
| | K+ channel subunit) - Homo | | | |
| | sapiens (Human), 538 aa. | | | |
| Q8TDK8 | Potassium channel TREK2 | 18 . . . 543 | 525/526 (99%) | 0.0 |
| | splice variant a - Homo | 18 . . . 543 | 525/526 (99%) | |
| | sapiens (Human), 543 aa. | | | |
| Q9JIS4 | Potassium channel subfamily | 1 . . . 543 | 520/544 (95%) | 0.0 |
| | K member 10 (Outward | 1 . . . 538 | 529/544 (96%) | |
| | rectifying potassium channel | | | |
| | protein TREK-2) (TREK-2 | | | |
| | K+ channel subunit) - Rattus | | | |
| | norvegicus (Rat), 538 aa. | | | |
| P97438 | Potassium channel subfamily | 22 . . . 404 | 247/384 (64%) | e−136 |
| | K member 2 (Outward | 2 . . . 369 | 301/384 (78%) | |
| | rectifying potassium channel | | | |
| | protein TREK-1) (Two-pore | | | |
| | potassium channel TPKC1) | | | |
| | (TREK-1 K+channel | | | |
| | subunit) - Mus musculus | | | |
| | (Mouse), 411 aa. | | | |

PFam analysis predicts that the NOV 19a protein contains the domains shown in the Table 19E.

TABLE 19E
Domain Analysis of NOV19a

| | NOV19a | Identities/Similarities | Expect |
|---|---|---|---|
| Pfam Domain | Match Region | for the Matched Region | Value |
| ion_trans | 158 . . . 323 | 41/231 (18%) | 0.046 |
| | | 119/231 (52%) | |

The NOV20 clone was analyzed, and the nucleotide and encoded polypeptide sequences are shown in Table 20A.

TABLE 20A
NOV20 Sequence Analysis

| | SEQ ID NO: 107 | 2958 bp | |
|---|---|---|---|
| NOV20a, | GCTCCTCCCCGCTGGCGGGGGGAGAAAGGGCAGGAGGCCTTCCGTCCCGGCTATAAAGGGCCCCGGA | | |
| CG146374-01 | | | |
| DNA Sequence | CCGCCGCGGCTCGCCTCGGCTTGCCTCGACACGCCTAGGCGCCCTCCGGCTCCGCCCTAGCCGCCGC | | |
| | GTCCCAGCTAGAGCTCCAGCGCCCGCTCAGGCCCCACTCGACCCTCTCGGGCCTCGGCTACTTGGAC | | |
| | TGCGGCGGAATATGGCGGCTCCGATGACTCCCGCGGCTCGGCCCGAGGACTACGAGGCGGCGCTCAA | | |
| | TGCCGCCCTGGCTGACGTGCCCGAACTGGCCAGACTCCTGGAGATCGACCCGTACTTGAAGCCCTAC | | |
| | GCCGTGGACTTCCAGCGCAGGTATAAGCAGTTTAGCCAAATTTTGAAGAACATTGGAGAAAATGAAG | | |
| | GTGGTATTGATAAGTTTTCCAGAGGCTATGAATCATTTGGCGTCCACAGATGTGCTGATGGTGGTTT | | |
| | ATACTGCAAAGAATGGGCCCCGGGAGCAGAAGGAGTTTTTCTTACTGGAGATTTTAATGGTTGGAAT | | |
| | CCATTTTCGTACCCATACAAAAAACTGGATTATGGAAAATGGGAGCTGTATATCCCACCAAAGCAGA | | |
| | ATAAATCTGTACTCGTGCCTCATGGATCCAAATTAAAGGTAGTTATTACTAGTAAAAGCGGAGAGAT | | |
| | CTTGTATCGTATTTCACCGTGGGCAAAGTATGTGGTTCGTGAAGGTGATAATGTGAATTATGATTGG | | |
| | ATACACTGGGATCCAGAACACTCATATGAGTTTAAGCATTCCAGACCAAAGAAGCCACGGAGTCTAA | | |
| | GAATTTATGAATCTCATGTGGGAATTTCTTCCCATGAAGGAAAAGTAGCTTCTTATAAACATTTTAC | | |
| | ATGCAATGTACTACCAAGAATCAAAGGCCTTGGATACAACTGCATTCAGTTGATGGCAATCATGGAG | | |
| | CATGCTTACTATGCCAGCTTTGGTTACCAAATCACAAGCTTCTTTGCAGCTTCCAGCCGTTATGGAT | | |
| | CACCTGAAGAGCTACAAGAACTGGTAGACACAGCTCATTCCATGGGTATCATAGTCCTCTTAGATGT | | |
| | GGTACACAGCCATGCTTCAAAAAATTCAGCAGATGGATTGAATATGTTTGATGGGACAGATTCCTGT | | |
| | TATTTTCATTCTGGACCTAGAGGGACTCATGATCTTTGGGATAGCAGATTGTTTGCCTACTCCAGGT | | |
| | TGAATATTTCAGACATCTAAGCCAATTAGAATCATGATTGTTTTGATTGCCAGAAATCCTTAAATCT | | |
| | GGGAAGTTTTAAGATTCCTTCTGTCAAACATAAGATGGTGGTTGGAAGAATATCGCTTTGATGGATT | | |
| | TCGTTTTGATGGTGTTACGTCCATGCTTTATCATCACCATGGAGTGGGTCAAGGTTTCTCAGGTGAT | | |
| | TACAGTGAATATTTCGGACTACAAGTAGATGAAGATGCCTTGACTTACCTCATGTTGGCAAATCATT | | |
| | TGGTTCACACGCTGTGTCCCGATTCTATAACAATAGCTGAGGATGTATCAGGAATGCCAGCTCTGTG | | |
| | CTCTCCAATTTCCCAGGGAGGGGGTGGTTTTGACTATCGACTAGCCATGGCAATTCCAGATAAGTGG | | |
| | ATTCAGCTACTTAAAGAGTTTAAAGATGAAGACTGGAACATGGGCGATATAGTATACACGCTCACAA | | |
| | ACAGGCGCTACCTTGAAAAGTGCATTGCTTATGCAGAGAGCCATGATCAGGCATTGGTTGGGGATAA | | |
| | GTCGCTGGCATTTTGGTTGATGGATGCCGAAATGTATACAAACATGAGTGTCCTGACTCCTTTTACT | | |
| | CCAGTTATTGATCGTGGAATACAGCTTCATAAAATGATTCGACTCATTACGCATGGGCTTGGTGGAG | | |
| | AAGGCTATCTCAATTTCATGGGTAATGAATTTGGGCATCCTGAATGGTTAGACTTCCCAAGAAAAGG | | |
| | AAATAATGAGAGTTACCATTATGCCAGGCGGCAGTTTCATTTAACTGACGACGACCTTCTTCGCTAC | | |
| | AAGTTCCTAAATAATTTTGACAGGGATATGAATAGATTGGAAGAAAGATATGGTTGGCTTGCAGCTC | | |
| | CACAGGCCTACGTGAGTGAAAAACATGAAGGCAATAAGATCATTGCTTTTGAAAGAGCAGGTCTTCT | | |
| | TTTCATTTTCAACTTCCATCCAAGCAAGAGCTACACTGACTACCGAGTTGGAACAGCATTGCCAGGG | | |
| | AAATTCAAAATTGTGCTAGATTCAGATGCAGCGGAATATGGAGGGCATCAGAGACTGGACCACAGCA | | |
| | CTGACTTTTTTTCTGAGGCTTTTGAACATAATGGGCGTCCCTATTCTCTTTTGGTGTACATTCCAAG | | |
| | CAGAGTGGCCCTCATCCTTCAGAATGTGGATCTGCCGAATTGAAGAGGCCTGATTTCAGCTCCACCA | | |
| | GATGCAGATTTGTGTTTTGTTTTCTTGTTATCACTGTCACACAGCTTATAACATGTATGCTTTTCAG | | |
| | AATACAGTTGTCTAGCCAAGCCATCAAGTGTCTGAAATTCAATATTGGTTTATGCAAATACAGCAAA | | |
| | CTTTTATTTAAGTAGATAGGAGAATATGTTTAAAATATTAGGAATCCTAGACCATATTTTCAAGTCA | | |
| | TCTTAGCAGCTAGGATTCTCAAATGGAAGTGTTATATATAATATGTTAAAAACATTTTGCTTTCCTG | | |
| | GCTAATTATTTGATCCTTTTAAATTCAAATTTGAATCATTTGTCATGTATGATTATTTCTGTTAAAT | | |
| | GTACACAGTATTTAAGATGGATATTTGGTGGCTCTATTTGTTCTGATATCTTTTGGTCTAAATTATG | | |
| | AGGTACCAAGATTGTTTCTTTGTTTCTTTTTTTCAAATTGTGTTTAGAAATACTGTAATAAATATGC | | |
| | AGTAGTGATATAAAGAATTATATCCAAGGTAATATAAAAGCCATTACGTATGAACTCAAAAAAAAAA | | |
| | AAAAAAAAAA | | |
| | ORF Start: ATG at 213 | | ORF Stop: TAA at 1224 |
| | SEQ ID NO: 108 | 337 aa | MW at 38247.8 kD |

| | |
|---|---|
| NOV20a, | MAAPMTPAARPEDYEAALNAALADVPELARLLEIDPYLKPYAVDFQRRYKQFSQILKNIGENEGGID |
| CG146374-01 | |
| Protein Sequence | KFSRGYESFGVHRCADGGLYCKEWAPGAEGVFLTGDFNGWNPFSYPYKKLDYGKWELYIPPKQNKSV |
| | LVPHGSKLKVVITSKSGEILYRISPWAKYVVREGDNVNYDWIHWDPEHSYEFKHSRPKKPRSLRIYE |
| | SHVGISSHEGKVASYKHFTCNVLPRIKGLGYNCIQLMAIMEHAYYASFGYQITSFFAASSRYGSPEE |
| | LQELVDTAHSMGIIVLLDVVHSHASKNSADGLNMFDGTDSCYFHSGPRGTHDLWDSRLFAYSRLNIS |
| | DI |

Further analysis of the NOV20a protein yielded the following properties shown in Table 20B.

TABLE 20B
Protein Sequence Properties NOV20a

| | |
|---|---|
| PSort | 0.7480 probability located in microbody (peroxisome); 0.6000 |
| analysis: | probability located in nucleus; 0.1000 probability located in |
| | mitochondrial matrix space; 0.1000 probability located in |
| | lysosome (lumen) |
| SignalP | No Known Signal Sequence Predicted |
| analysis: | |

A search of the NOV20a protein against the Geneseq database, a proprietary database that contains sequences published in patents and patent publication, yielded several homologous proteins shown in Table 20C.

TABLE 20C
Geneseq Results for NOV20a

| Geneseq | Protein/Organism/Length | NOV20a Residues/ | Identities/Similarities | Expect |
|---|---|---|---|---|
| Identifier | [Patent #, Date] | Match Residues | for the Matched Region | Value |
| AAB90803 | Human shear stress-response | 1 . . . 330 | 328/330 (99%) | 0.0 |
| | protein SEQ ID NO: 106 - | 1 . . . 330 | 329/330 (99%) | |
| | Homo sapiens, 702 aa. | | | |
| | [WO200125427-A1, | | | |
| | 12-APR-2001] | | | |
| ABB60350 | Drosophila melanogaster | 22 . . . 329 | 170/314 (54%) | e−102 |
| | polypeptide SEQ ID NO | 1 . . . 314 | 227/314 (72%) | |
| | 7842 - Drosophila | | | |
| | melanogaster, 865 aa. | | | |
| | [WO200171042-A2, | | | |
| | 27-SEP-2001] | | | |
| AAB49603 | Glycogen branching enzyme | 31 . . . 329 | 175/305 (57%) | 1e−98 |
| | amino acid sequence - | 12 . . . 314 | 228/305 (74%) | |
| | Aspergillus nidulans, 686 aa. | | | |
| | [JP2000279180-A, | | | |
| | 10-OCT-2000] | | | |
| AAG39093 | Arabidopsis thaliana protein | 30 . . . 329 | 161/302 (53%) | 3e−92 |
| | fragment SEQ ID NO: 48322 | 22 . . . 321 | 214/302 (70%) | |
| | - Arabidopsis thaliana, 721 | | | |
| | aa. [EP1033405-A2, | | | |
| | 06-SEP-2000] | | | |
| AAG39092 | Arabidopsis thaliana protein | 30 . . . 329 | 161/302 (53%) | 3e−92 |
| | fragment SEQ ID NO: 48321 | 159 . . . 458 | 214/302 (70%) | |
| | - Arabidopsis thaliana, 858 | | | |
| | aa. [EP1033405-A2, | | | |
| | 06-SEP-2000] | | | |

In a BLAST search of public sequence datbases, the NOV20a protein was found to have homology to the proteins shown in the BLASTP data in Table 20D.

TABLE 20D
Public BLASTP Results for NOV20a

| Protein | | | | |
|---|---|---|---|---|
| Accession | | NOV20a Residues/ | Identities/Similarities | Expect |
| Number | Protein/Organism/Length | Match Residues | for the Matched Portion | Value |
| Q96EN0 | Similar to glucan | 1 . . . 330 | 330/330 (100%) | 0.0 |
| | (1,4-alpha-), branching | 1 . . . 330 | 330/330 (100%) | |
| | enzyme 1 (glycogen | | | |
| | branching enzyme, Andersen | | | |
| | disease, glycogen storage | | | |
| | disease type IV) - Homo | | | |
| | sapiens (Human), 702 aa. | | | |
| Q04446 | 1,4-alpha-glucan branching | 1 . . . 330 | 328/330 (99%) | 0.0 |
| | enzyme (EC 2.4.1.18) | 1 . . . 330 | 329/330 (99%) | |
| | (Glycogen branching | | | |
| | enzyme) (Brancher enzyme) | | | |
| | - Homo sapiens (Human), | | | |
| | 702 aa. | | | |
| Q9D6Y9 | 2310045H19Rik protein | 1 . . . 330 | 291/330 (88%) | e−179 |
| | (RIKEN cDNA 2310045H19 | 1 . . . 330 | 310/330 (93%) | |
| | gene) - Mus musculus | | | |
| | (Mouse), 702 aa. | | | |
| AAF58416 | CG4023-PA - Drosophila | 22 . . . 329 | 170/314 (54%) | e−102 |
| | melanogaster (Fruit fly), 685 | 1 . . . 314 | 227/314 (72%) | |
| | aa. | | | |
| Q9V6K7 | GG4023 protein - Drosophila | 22 . . . 329 | 170/314 (54%) | e−102 |
| | melanogaster (Fruit fly), 865 | 1 . . . 314 | 227/314 (72%) | |
| | aa. | | | |

PFam analysis predicts that the NOV20a protein contains the domains shown in the Table 20E.

TABLE 20E
Domain Analysis of NOV20a

| | | Identities/ | |
|---|---|---|---|
| | | Similarities | |
| | NOV20a | for the Matched | |
| Pfam Domain | Match Region | Region | Expect Value |
| isoamylase_N | 73 . . . 168 | 31/123(25%) | 5.1e−11 |
| | | 64/123 (52%) | |

### Example 21

The NOV21 clone was analyzed, and the nucleotide and encoded polypeptide sequences are shown in Table 21A.

TABLE 21A
NOV21 Sequence Analysis

| | SEQ ID NO: 109 | 885 bp | |
|---|---|---|---|
| NOV21a. | TGGATGCTGGCGGTCCTCTACCTGGTCTGGCTCTATTGGGATAGAAACATACCCAGGGCTGGTGGAA | | |
| CG146403-01 | | | |
| DNA Sequence | GGCGTTCGGAGTGGATAAGGAACCGGGCAATTTGGAGACAACTAAGGGATTATTATCCTGTCAAGCT | | |
| | GGTGAAAACAGCAGAGCTGCCCCCGGATCGGAACTACGTGCTGGGCGCCCACCCTCATGGGATCATG | | |
| | TGTACAGGCTTCCTCTGTAATTTCTCCACCGAGAGCAATGGCTTCTCCCAGCTCTTCCCGGGGCTCC | | |
| | GGCCCTGGTTAGCCGTGCTGGCTGGCCTCTTCTACCTCCCGGTCTATCGCGACTACATCATGTCCTT | | |
| | TGGTCTCTGTCCGGTGAGCCGCCAGAGCCTGGACTTCATCCTGTCCCAGCCCCAGCTCGGGCAGGCC | | |
| | GTGGTCATCATGGTGGGGGGTGCGCACGAGGCCCTGTATTCAGTCCCCGGGGAGCACTGCCTTACGC | | |
| | TCCAGAAGCGCAAAGGCTTCGTGCGCCTGGCGCTGAGGCACGGGGCGTCCCTGGTGCCCGTGTACTC | | |
| | CTTTGGGGAGAATGACATCTTTAGACTTAAGGCTTTTGCCACAGGCTCCTGGCAGCATTGGTGCCAG | | |
| | CTCACCTTCAAGAAGCTCATGGGCTTCTCTCCTTGCATCTTCTGGGGTCGCGGTCTCTTCTCAGCCA | | |
| | CCTCCTGGGGCCTGCTGCCCTTTGCTGTGCCCATCACCACTGTGGGTGAGCCCATCCCCGTCCCCCA | | |
| | GCGCCTCCACCCCACCGAGGAGGAAGTCAATCACTATCACGCCCTCTACATGACGGCCCTGGAGCAG | | |
| | CTCTTCGAGGAGCACAAGGAAAGCTGTGGGGTCCCCGCTTCCACCTGCCTCACCTTCATCTAGGCCT | | |
| | GGCCGCGGCCTTTC | | |
| | ORF Start: ATG at 4 | | ORF Stop: TAG at 865 |
| | SEQ ID NO: 110 | 287 aa | MW at 32641.7 kD |

| | |
|---|---|
| NOV21a, | MLAVLYLVWLYWDRNIPRAGGRRSEWIRNRAIWRQLRDYYPVKLVKTAELPPDRNYVLGAHPHGIMC |
| CG146403-01 | |
| Protein Sequence | TGFLCNFSTESNGFSQLFPGLRPWLAVLAGLFYLPVYRDYIMSFGLCPVSRQSLDFILSQPQLGQAV |
| | VIMVGGAHEALYSVPGEHCLTLQKRKGFVRLALRHGASLVPVYSFGENDIFRLKAFATGSWQHWCQL |
| | TFKKLMGFSPCIFWGRGLFSATSWGLLPFAVPITTVGEPIPVPQRLHPTEEEVNHYHALYMTALEQL |
| | FEEHKESCGVPASTCLTFI |

Further analysis of the NOV21a protein yielded the following properties shown in Table 21B.

TABLE 21B
Protein Sequence Properties NOV21a

| | |
|---|---|
| PSort analysis: | 0.5500 probability located in endoplasmic |
| | reticulum (membrane); 0.3814 probability |
| | located in lysosome (lumen); 0.3200 probability |
| | located in microbody (peroxisome); 0.1000 probability |
| | located in endoplasmic reticulum (lumen) |
| SignalP analysis: | Cleavage site between residues 22 and 23 |

A search of the NOV21a protein against the Geneseq database, a proprietary database that contains sequences published in patents and patent publication, yielded several homologous proteins shown in Table 21C.

TABLE 21C
Geneseq Results for NOV21a

| | | NOV21a | | |
|---|---|---|---|---|
| | | Residues/ | Identities/ | |
| Geneseq | Protein/Organism/Length | Match | Similarities for the | Expect |
| Identifier | [Patent #, Date] | Residues | Matched Region | Value |
| AAM80262 | Human protein SEQ ID NO | 43 . . . 237 | 195/195 (100%) | e−115 |
| | 3908 - Homo sapiens, 223 | 29 . . . 223 | 195/195 (100%) | |
| | aa. [WO200157190-A2, | | | |
| | 09-AUG-2001] | | | |
| ABB75677 | Breast protein-eukaryotic | l . . . 284 | 158/285 (55%) | 1e−97 |
| | conserved gene 1 | 101 . . . 385 | 218/285 (76%) | |
| | (BSTP-ECG1) protein - | | | |
| | Homo sapiens, 388 aa. | | | |
| | [WO200208260-A2, | | | |
| | 31-JAN-2002] | | | |
| AAB66170 | Protein of the invention #82 - | 1 . . . 284 | 158/285 (55%) | 1e−97 |
| | Unidentified, 388 aa. | 101 . . . 385 | 218/285 (76%) | |
| | [WO200078961-A1, | | | |
| | 28-DEC-2000] | | | |
| AAU29191 | Human PRO polypeptide | 1 . . . 284 | 158/285 (55%) | 1e−97 |
| | sequence #168 - Homo | 101 . . . 385 | 218/285 (76%) | |
| | sapiens, 388 aa. | | | |
| | [WO200168848-A2, | | | |
| | 20-SEP-2001] | | | |
| AAY99421 | Human PR01433 (UNQ738) | 1 . . . 284 | 158/285 (55%) | 1e−97 |
| | amino acid sequence SEQ ID | 101 . . . 385 | 218/285 (76%) | |
| | NO: 292 - Homo sapiens, 388 | | | |
| | aa. [WO200012708-A2, | | | |
| | 09-MAR-2000] | | | |

In a BLAST search of public sequence datbases, the NOV21a protein was found to have homology to the proteins shown in the BLASTP data in Table 21D.

TABLE 21D
Public BLASTP Results for NOV21a

| | | NOV21a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9UDW7 | WUGSC: H_DJ0747G18.5 | 43 . . . 287 | 244/246 (99%) | e−145 |
| | protein - Homo sapiens | 16 . . . 261 | 244/246 (99%) | |
| | (Human), 261 aa (fragment). | | | |
| CAD38961 | Hypothetical protein - Homo | 1 . . . 284 | 158/285 (55%) | 3e−97 |
| | sapiens (Human), 434 aa | 147 . . . 431 | 218/285 (76%) | |
| | (fragment). | | | |
| Q96PD7 | Diacyiglycerol | 1 . . . 284 | 158/285 (55%) | 3e−97 |
| | acyltransferase 2 | 1O1 . . . 385 | 218/285 (76%) | |
| | (Hypothetical 43.8 kDa | | | |
| | protein) - Homo sapiens | | | |
| | (Human), 388 aa. | | | |
| Q9BYE5 | GSl999full protein - Homo | 1 . . . 284 | 158/285 (55%) | 3e−97 |
| | sapiens (Human), 297 aa. | 1O . . . 294 | 218/285 (76%) | |
| Q9DCV3 | 0610010B06Rik protein | 1 . . . 284 | 159/285 (55%) | 8e−97 |
| | (Diacyiglycerol acyltransferase | 101 . . . 385 | 217/285 (75%) | |
| | 2) - Mus | | | |
| | musculus (Mouse), 388 aa. | | | |

### Example 22

The NOV22 clone was analyzed, and the nucleotide and encoded polypeptide sequences are shown in Table 22A.

TABLE 22A
NOV22 Sequence Analysis

| | SEQ ID NO: 111 | 1135 bp | |
|---|---|---|---|
| NOV22a, | CACAGTAAGAGATTATAGCAAAGCATCTATAATCAACTCAGCTTAAGAAGTTTTGACCTTCTGGTTA | | |
| CG146513-01 | | | |
| DNA Sequence | GGCTTCTTGCCACAACAGAACAGCACCATAACCATGGCTTTCTTCTCCCGACTGAATCTCCAGGAGG | | |
| | GCCTCCAAACCTTCTTTGTTTTGCAATGGATCCCAGTCTATATATTTTTAGGAGCTATTCCCATTCT | | |
| | CCTTATACCCTACTTTCTGTTATTCAGTAAGTTCTGGCCCTTGGCTGTGCTCTCCTTAGCCTGGCTC | | |
| | ACCTATGATTGGAACACCCACAGTCAAGGTGGCAGGCGTTCAGCTTGGGTACGAAACTGGACCCTAT | | |
| | GGAAGTATTTCCGAAATTACTTCCCAGTACAGCTGGTGAAGACTCATGATCTTTCTCCCAAACACAA | | |
| | CTACATCATTGCCAATCACCCCCATGGCATTCTCTCTTTTGGTGTCTTCATCAACTTTGCCACTGAG | | |
| | GCCACTGGCATTGCTCGGATTTTCCCATCCATCACTCCCTTTGTAGGGACCTTAGAAAGGATATTTT | | |
| | GGATCCCAATTGTGCGAGAATATGTGATGTCAATGGGTGTGTGCCCTGTGAGTAGCTCAGCCTTGAA | | |
| | GTACTTGCTGACCCAGAAAGGCTCAGGCAATGCCGTGGTTATTGTGGTGGGTGGAGCTGCTGAAGCT | | |
| | CTCTTGTGCCGACCAGGAGCCTCCACTCTCTTCCTCAAGCAGCGTAAAGGTTTTGTGAAGATGGCAC | | |
| | TGCAAACAGGGGCATACCTTGTCCCTTCATATTCCTTTGGTGAGAACGAAGTTTTCAATCAGGAGAC | | |
| | CTTCCCTGAGGGCACGTGGTTAAGGTTGTTCCAAAAAACCTTCCAGGACACATTCAAAAAAATCCTG | | |
| | GGACTAAATTTCTGTACCTTCCATGGCCGGGGCTTCACTCGCGGATCCTGGGGCTTCCTGCCTTTCA | | |
| | ATCGGCCCATTACCACTGTTGGGGAACCCCTTCCAATTCCCAGGATTAAGAGGCCAAACCAGAAGAC | | |
| | AGTAGACAAGTATCACGCACTCTACATCAGTGCCCTGCGCAAGCTCTTTGACCAACACAAAGTTGAA | | |
| | TATGGCCTCCCTGAGACCCAAGAGCTGACAATTACATAACAGGAGCCACATTCCCCATTGATC | | |
| | ORF Start: ATG at 101 | | ORF Stop: TAA at 1109 |
| | SEQ ID NO: 112 | 336 aa | MW at 38493.6 kD |

| | |
|---|---|
| NOV22a, | MAFFSRLNLQEGLQTFFVLQWIPVYIFLGAIPILLIPYFLLFSKFWPLAVLSLAWLTYDWNTHSQGG |
| CG146513-01 | |
| Protein Sequence | RRSAWVRNWTLWKYFRNYFPVQLVKTHDLSPKHNYIIANHPHGILSFGVFINFATEATGIARIFPSI |
| | TPFVGTLERIFWIPIVREYVMSMGVCPVSSSALKYLLTQKGSGNAVVIVVGGAAEALLCRPGASTLF |
| | LKQRKGFVKMALQTGAYLVPSYSFGENEVFNQETFPEGTWLRLFQKTFQDTFKKILGLNFCTFHGRG |
| | FTRGSWGFLPFNRPITTVGEPLPIPRIKRPNQKTVDKYHALYISALRKLFDQHKVEYGLPETQELTI |
| | T |

Further analysis of the NOV22a protein yielded the following properties shown in Table 22B.

TABLE 22B
Protein Sequence Properties NOV22a

| | |
|---|---|
| PSort analysis: | 0.6850 probability located in plasma membrane; |
| | 0.6400 probability located in endoplasmic reticulum |
| | (membrane); 0.3880 probability located in microbody |
| | (peroxisome); 0.3700 probability located in Golgi body |
| SignalP analysis: | Cleavage site between residues 65 and 66 |

A search of the NOV22a protein against the Geneseq database, a proprietary database that contains sequences published in patents and patent publication, yielded several homologous proteins shown in Table 22C.

TABLE 22C
Geneseq Results for NOV22a

| | | NOV22a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAM06866 | Human foetal protein, SEQ | 1 . . . 216 | 211/216 (97%) | e−124 |
| | ID NO: 1074 - Homo | 1 . . . 216 | 214/216 (98%) | |
| | sapiens, 225 aa. | | | |
| | [WO200155339-A2, | | | |
| | 02-AUG-2001] | | | |
| ABB75677 | Breast protein-eukaryotic | 1 . . . 335 | 171/337 (50%) | e−101 |
| | conserved gene 1 | 56 . . . 387 | 237/337 (69%) | |
| | (BSTP-ECG1) protein - | | | |
| | Homo sapiens, 388 aa. | | | |
| | [WO200208260-A2, | | | |
| | 31-JAN-2002] | | | |
| AAB66170 | Protein of the invention #82 - | 1 . . . 335 | 171/337 (50%) | e−101 |
| | Unidentified, 388 aa. | 56 . . . 387 | 237/337 (69%) | |
| | [WO200078961-A1, | | | |
| | 28-DEC-2000] | | | |
| AAU29191 | Human PRO polypeptide | 1 . . . 335 | 171/337 (50%) | e−101 |
| | sequence #168 - Homo | 56 . . . 387 | 237/337 (69%) | |
| | sapiens, 388 aa. | | | |
| | [WO200168848-A2, | | | |
| | 20-SEP-2001] | | | |
| AAY99421 | Human PR01433 (UNQ738) | 1 . . . 335 | 171/337 (50%) | e−101 |
| | amino acid sequence SEQ ID | 56 . . . 387 | 237/337 (69%) | |
| | NO: 292 - Homo sapiens, 388 | | | |
| | aa. [WO200012708-A2, | | | |
| | 09-MAR-2000] | | | |

In a BLAST search of public sequence datbases, the NOV22a protein was found to have homology to the proteins shown in the BLASTP data in Table 22D.

TABLE 22D
Public BLASTP Results for NOV22a

| | | NOV22a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9DCV3 | 0610010B06Rik protein | 1 . . . 335 | 172/337(51%) | e−101 |
| | (Diacyiglycerol | 56 . . . 387 | 238/337 (70%) | |
| | acyltransferase 2) - Mus | | | |
| | musculus (Mouse), 388 aa. | | | |
| CAD38961 | Hypothetical protein - Homo | 1 . . . 335 | 171/337 (50%) | e−100 |
| | sapiens (Human), 434 aa | 102 . . . 433 | 237/337 (69%) | |
| | (fragment). | | | |
| Q96PD7 | Diacyiglycerol | 1 . . . 335 | 171/337 (50%) | e−100 |
| | acyltransferase 2 | 56 . . . 387 | 237/337 (69%) | |
| | (Hypothetical 43.8 kDa | | | |
| | protein) - Homo sapiens | | | |
| | (Human), 388 aa. | | | |
| Q8TAB1 | BA351K23.5 (Novel protein) - | 38 . . . 335 | 161/299 (53%) | 2e−98 |
| | Homo sapiens (Human), | 1 . . . 295 | 221/299 (73%) | |
| | 296 aa (fragment). | | | |
| Q9BYE5 | GS1999full protein - Homo | 39 . . . 335 | 161/299 (53%) | 4e−96 |
| | sapiens (Human), 297 aa. | 2 . . . 296 | 217/299 (71%) | |

### Example 23

The NOV23 clone was analyzed, and the nucleotide and encoded polypeptide sequences are shown in Table 23A.

TABLE 23A
NOV23 Sequence Analysis

| | SEQ ID NO: 113 | 1022 bp | |
|---|---|---|---|
| NOV23a, | ACTGTTCTGAGATCTTTGCCTCCCTCAGGCTCCCGAGAATCATGGCTCATTCCAAGCAGCCTAGTCA | | |
| CG146522-01 | | | |
| DNA Sequence | CTTCCAGAGTCTGATGCTTCTGCAGTGGCCTTTGAGCTACCTTGCCATCTTGTTCGTCTACCTGCTG | | |
| | TTTACATCCTTGTGGCCGCTACCAGTGCTTTACTTTGCCTGGTTGTTCCTGGACTGGAAGACCCCAG | | |
| | AGCGAGGTGGCAGGCGTTCGGCCTGGGTAAGGAACTGGTGTGTCTGGACCCACATCAGGGACTATTT | | |
| | CCCCATTATCCTGAAGACAAAGGACCTATCACCTGAGCACAACTACCTCATGGGGGTTCACCCCCAT | | |
| | GGCCTCCTGACCTTTGGCGCCTTCTGCAACTTCTGCACTGAGGCCACAGGCTTCTCGAAGACCTTCC | | |
| | CAGGCATCACTCCTCACTTGGCCACGCTGTCCTGGTTCTTCAAGATCCCCTTTGTTAGGGAGTACCT | | |
| | CATGGCCAAAGGTGTGTGCTCTGTGAGCCAGCCAGCCATCAACTATCTGCTGAGCCATGGCACTGGC | | |
| | AACCTCGTGGGCATTGTAGTGGGAGGTGTGGGTGAGGCCCTGCAAAGTGTGCCCAACACCACCACCC | | |
| | TCATCCTCCAGAAGCGCAAGGGGTTCGTGCGCACAGCCCTCCAGCATGGGGCTCATCTGGTCCCCAC | | |
| | CTTCACTTTTGGGGAAACTGAGGTGTATGATCAGGTGCTGTTCCATAAGGATAGCAGGATGTACAAG | | |
| | TTCCAGAGCTGCTTCCGCCGTATCTTTGGTTTCTACTGTTGTGTCTTCTATGGACAAAGCTTCTGTC | | |
| | AAGGCTCCACTGGGCTCCTGCCATACTCCAGGCCTATTGTCACTGTTGGGGAGCCTCTGCCACTGCC | | |
| | CCAAATTGAAAAGCCAAGCCAGGAGATGGTGGACAAATACCATGCACTTTATATGGATGCTCTGCAC | | |
| | AAACTGTTCGACCAGCATAAGACCCACTATGGCTGCTCAGAGACCCAAAAGCTGTTTTTCCTGTGAA | | |
| | TGAAGGTACTGCATGCC | | |
| | ORF Start: ATG at 42 | | ORF Stop: TGA at 1002 |
| | SEQ ID NO: 114 | 320 aa | MW at 36773.5 kD |

| | |
|---|---|
| NOV23a, | MAHSKQPSHFQSLMLLQWPLSYLAILFVYLLFTSLWPLPVLYFAWLFLDWKTPERGGRRSAWVRNWC |
| CG146522-01 | |
| Protein Sequence | VWTHIRDYFPIILKTKDLSPEHNYLMGVHPHGLLTFGAFCNFCTEATGFSKTFPGITPHLATLSWFF |
| | KIPFVREYLMAKGVCSVSQPAINYLLSHGTGNLVGIVVGGVGEALQSVPNTTTLILQKRKGFVRTAL |
| | QHGAHLVPTFTFGETEVYDQVLFHKDSRMYKFQSCFRRIFGFYCCVFYGQSFCQGSTGLLPYSRPIV |
| | TVGEPLPLPQIEKPSQEMVDKYHALYMDALHKLFDQHKTHYGCSETQKLFFL |

Further analysis of the NOV23a protein yielded the following properties shown in Table 23B.

TABLE 23B
Protein Sequence Properties NOV23a

| | |
|---|---|
| PSort analysis: | 0.7284 probability located in outside; 0.3880 |
| | probability located in microbody (peroxisome); |
| | 0.1000 probability located in endoplasmic reticulum |
| | (membrane); 0.1000 probability located in |
| | endoplasmic reticulum (lumen) |
| SignalP analysis: | Cleavage site between residues 43 and 44 |

A search of the NOV23a protein against the Geneseq database, a proprietary database that contains sequences published in patents and patent publication, yielded several homologous proteins shown in Table 23C.

TABLE 23C
Geneseq Results for NOV23a

| | | NOV23a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB75677 | Breast protein-eukaryotic | 4 . . . 317 | 165/324 (50%) | 1e−93 |
| | conserved gene 1 | 62 . . . 385 | 224/324 (68%) | |
| | (BSTP-ECG1) protein - | | | |
| | Homo sapiens, 388 aa. | | | |
| | [WO200208260-A2, | | | |
| | 31-JAN-2002] | | | |
| AAB66170 | Protein of the invention #82 - | 4 . . . 317 | 165/324 (50%) | 1e−93 |
| | Unidentified, 388 aa. | 62 . . . 385 | 224/324 (68%) | |
| | [WO200078961-A1, | | | |
| | 28-DEC-2000] | | | |
| AAU29191 | Human PRO polypeptide | 4 . . . 317 | 165/324 (50%) | 1e−93 |
| | sequence #168 - Homo | 62 . . . 385 | 224/324 (68%) | |
| | sapiens, 388 aa. | | | |
| | [WO200168848-A2, | | | |
| | 20-SEP-2001] | | | |
| AAY99421 | Human PR01433 (UNQ738) | 4 . . . 317 | 165/324 (50%) | 1e−93 |
| | amino acid sequence SEQ ID | 62 . . . 385 | 224/324 (68%) | |
| | NO: 292 - Homo sapiens, 388 | | | |
| | aa. [WO200012708-A2, | | | |
| | 09-MAR-2000] | | | |
| AAY94889 | Human protein clone | 11 . . . 319 | 144/318 (45%) | 3e−74 |
| | HP02485 - Homo sapiens, 334 aa. | 16 . . . 333 | 200/318 (62%) | |
| | [WO200005367-A2, | | | |
| | 03-FEB-2000] | | | |

In a BLAST search of public sequence datbases, the NOV23a protein was found to have homology to the proteins shown in the BLASTP data in Table 23D.

TABLE 23D
Public BLASTP Results for NOV23a

| | | NOV23a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8TAB1 | BA351K23.5 (Novel protein) - | 30 . . . 317 | 163/291 (56%) | 5e−96 |
| | Homo sapiens (Human), | 3 . . . 293 | 214/291 (73%) | |
| | 296 aa (fragment). | | | |
| Q9DCV3 | 0610010B06Rik protein | 4 . . . 317 | 166/324 (51%) | 2e−93 |
| | (Diacyiglycerol | 62 . . . 385 | 225/324 (69%) | |
| | acyltransferase 2) - Mus | | | |
| | musculus (Mouse), 388 aa. | | | |
| CAD38961 | Hypothetical protein - Homo | 4 . . . 317 | 165/324 (50%) | 3e−93 |
| | sapiens (Human), 434 aa | 108 . . . 431 | 224/324 (68%) | |
| | (fragment). | | | |
| Q96PD7 | Diacyiglycerol | 4 . . . 317 | 165/324 (50%) | 3e−93 |
| | acyltransferase 2 | 62 . . . 385 | 224/324 (68%) | |
| | (Hypothetical 43.8 kDa | | | |
| | protein) - Homo sapiens | | | |
| | (Human), 388 aa. | | | |
| Q9BYE5 | GS1999full protein - Homo | 28 . . . 317 | 156/294 (53%) | 1e−89 |
| | sapiens (Human), 297 aa. | 1 . . . 294 | 210/294 (71%) | |

### Example 24

The NOV24 clone was analyzed, and the nucleotide and encoded polypeptide sequences are shown in Table 24A.

TABLE 24A
NOV24 Sequence Analysis

| | SEQ ID NO: 115 | 1056 bp | |
|---|---|---|---|
| NOV24a, | CATTTTCCAAAGGTGTCACAGGAAGAGCATGGCAGAGCTGGGACTGGGAGCCAGGTCACCATGGCTT | | |
| CG146531-01 | | | |
| DNA Sequence | TCTTCTCCCGACTGAATCTCCAGGAGGGCCTCCAAACCTTCTTTGTTTTGCAATGGATCCCAGTCTA | | |
| | TATATTTTTAGGTTTGTTCGTCTACCTGCTGTTTACATCCTTGTGGCCGCTACCAGTGCTTTACTTT | | |
| | GCCTGGTTGTTCCTGGACTGGAAGACCCCAGAGCGAGGTGGCAGGCGTTCGGCCTGGGTAAGGAACT | | |
| | GGTGTGTCTGGACCCACATCAGGGACTATTTCCCCATTCAGATCCTGAAGACAAAGGACCTATCACC | | |
| | TGAGCACAACTACCTCATGGGGGTTCACCCCCATGGCCTCCTGACCTTTGGCGCCTTCTGCAACTTC | | |
| | TGCACTGAGGCCACAGGCTTCTCGAAGACCTTCCCAGGCATCACTCCTCACTTGGCCACGCTGTCCT | | |
| | GGTTCTTCAAGATCCCCTTTGTTAGGGAGTACCTCATGGCCAAAGGTGTGTGCTCTGTGAGCCAGCC | | |
| | AGCCATCAACTATCTGCTGAGCCATGGCACTGGCAACCTCGTGGGCATTGTAGTGGGAGGTGTGGGT | | |
| | GAGGCCCTGCAAAGTGTGCCCAACACCACCACCCTCATCCTCCAGAAGCGCAAGGGGTTCGTGCGCA | | |
| | CAGCCCTCCAGCATGGGGCTCATCTGGTCCCCACCTTCACTTTTGGGGAAACTGAGGTGTATGATCA | | |
| | GGTGCTGTTCCATAAGGATAGCAGGATGTACAAGTTCCAGAGCTGCTTCCGCCGTATCTTTGGTTTC | | |
| | TACTGTTGTGTCTTCTATGGACAAAGCTTCTGTCAAGGCTCCACTGGGCTCCTGCCATACTCCAGGC | | |
| | CTATTGTCACTGTTGGGGAGCCTCTGCCACTGCCCCAAATTGAAAAGCCAAGCCAGGAGATGGTGGA | | |
| | CAAATACCATGCACTTTATATGGATGCTCTGCACAAACTGTTCGACCAGCATAAGACCCACTATGGC | | |
| | TGCTCAGAGACCCAAAAGCTGTTTTTCCTGTGAATGAAGGTACTGCATCCC | | |
| | ORF Start: ATG at 61 | | ORF Stop: TGA at 1036 |
| | SEQ ID NO: 116 | 325 aa | MW at 37453.3 kD |

| | |
|---|---|
| NOV24a, | MAFFSRLNLQEGLQTFFVLQWIPVYIFLGLFVYLLFTSLWPLPVLYFAWLFLDWKTPERGGRRSAWV |
| CG146531-01 | |
| Protein Sequence | RNWCVWTHIRDYFPIQILKTKDLSPEHNYLMGVHPHGLLTFGAFCNFCTEATGFSKTFPGITPHLAT |
| | LSWFFKIPFVREYLMAKGVCSVSQPAINYLLSHGTGNLVGIVVGGVGEALQSVPNTTTLILQKRKGF |
| | VRTALQHGAHLVPTFTFGETEVYDQVLFHKDSRMYKFQSCFRRIFGFYCCVFYGQSFCQGSTGLLPY |
| | SRPIVTVGEPLPLPQIEKPSQEMVDKYHALYMDALHKLFDQHKTHYGCSETQKLFFL |

Further analysis of the NOV24a protein yielded the following properties shown in Table 24B.

TABLE 24B
Protein Sequence Properties NOV24a

| | |
|---|---|
| PSort analysis: | 0.8200 probability located in outside; |
| | 0.3880 probability located in microbody |
| | (peroxisome); 0.1000 probability located in |
| | endoplasmic reticulum (membrane); 0.1000 |
| | probability located in endoplasinic reticulum (lumen) |
| SignalP analysis: | Cleavage site between residues 47 and 48 |

A search of the NOV24a protein against the Geneseq database, a proprietary database that contains sequences published in patents and patent publication, yielded several homologous proteins shown in Table 24C.

Table 24C
Geneseq Results for NOV24a

| | | NOV24a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB75677 | Breast protein-eukaryotic | 1 . . . 322 | 166/330 (50%) | 2e−96 |
| | conserved gene 1 | 56 . . . 385 | 230/330 (69%) | |
| | BSTP-ECG1) protein - | | | |
| | Homo sapiens, 388 aa. | | | |
| | [WO200208260-A2, | | | |
| | 31-JAN-2002] | | | |
| AAB66170 | Protein of the invention #82 - | 1 . . . 322 | 166/330 (50%) | 2e−96 |
| | Unidentified, 388 aa. | 56 . . . 385 | 230/330 (69%) | |
| | [WO200078961-A1, | | | |
| | 28-DEC-2000] | | | |
| AAU29191 | Human PRO polypeptide | 1 . . . 322 | 166/330 (50%) | 2e−96 |
| | sequence #168 - Homo | 56 . . . 385 | 230/330 (69%) | |
| | sapiens, 388 aa. | | | |
| | [WO200168848-A2, | | | |
| | 20-SEP-2001] | | | |
| AAY99421 | Human PRO 1433 (UNQ738) | 1 . . . 322 | 166/330 (50%) | 2e−96 |
| | amino acid sequence SEQ ID | 56 . . . 385 | 230/330 (69%) | |
| | NO: 292 - Homo sapiens, 388 | | | |
| | aa. [WO200012708-A2, | | | |
| | 09-MAR-2000] | | | |
| AAY94889 | Human protein clone | 13 . . . 324 | 147/321 (45%) | 2e−75 |
| | HP02485 - Homo sapiens, 334 aa. | 15 . . . 333 | 200/321 (61%) | |
| | [WO200005367-A2, | | | |
| | 03-FEB-2000] | | | |

In a BLAST search of public sequence datbases, the NOV24a protein was found to have homology to the proteins shown in the BLASTP data in Table 24D.

TABLE 24D
Public BLASTP Results for NOV24a

| | | NOV24a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q8TAB1 | BA351K23.5 (Novel protein) - | 34 . . . 322 | 163/291 (56%) | 1e−97 |
| | Homo sapiens (Human), | 3 . . . 293 | 215/291 (73%) | |
| | 296 aa (fragment). | | | |
| CAD38961 | Hypothetical protein - Homo | 1 . . . 322 | 166/330 (50%) | 6e−96 |
| | sapiens (Human), 434 aa | 102 . . . 431 | 230/330 (69%) | |
| | (fragment). | | | |
| Q9DCV3 | 0610010B06Rik protein | 1 . . . 322 | 167/330 (50%) | 6e−96 |
| | (Diacylglycerol | 56 . . . 385 | 230/330 (69%) | |
| | acyltransferase 2) - Mus | | | |
| | musculus (Mouse), 388 aa. | | | |
| Q96PD7 | Diacyiglycerol | 1 . . . 322 | 166/330 (50%) | 6e−96 |
| | acyltransferase 2 | 56 . . . 385 | 230/330 (69%) | |
| | (Hypothetical 43.8 kDa | | | |
| | protein) - Homo sapiens | | | |
| | (Human), 388 aa. | | | |
| Q9BYE5 | GS1999full protein - Homo | 32 . . . 322 | 157/294 (53%) | 1e−91 |
| | sapiens (Human), 297 aa. | 1 . . . 294 | 211/294 (71%) | |

### Example 25

The NOV25 clone was analyzed, and the nucleotide and encoded polypeptide sequences are shown in Table 25A.

TABLE 25A
NOV25 Sequence Analysis

| | SEQ ID NO: 117 | 951 bp | |
|---|---|---|---|
| NOV25a, | ATGGGGCTTCGGGCAGGCCCCATCCTGCTTCTGCTGCTGTGGCTGCTGCCAGGGGCCCATTGGGATG | | |
| CG147274-01 | | | |
| DNA Sequence | TGCTGCCTTCAGAATGCGGCCACTCCAAGGAGGCCGGGAGGATTGTGGGAGGCCAAGACACCCAGGA | | |
| | AGGACGCTGGCCGTGGCAGGTTGGCCTGTGGTTGACCTCAGTGGGGCATGTATGTGGGGGCTCCCTC | | |
| | ATCCACCCACGCTGGGTGCTCACAGCCGCCCACTGCTTCCTGAGGTCTGAGGATCCCGGGCTCTACC | | |
| | ATGTTAAAGTCGGAGGGCTGACACCCTCACTTTCAGAGCCCCACTCGGCCTTGGTGGCTGTGAGGAG | | |
| | GCTCCTGGTCCACTCCTCATACCATGGGACCACCACCAGCGGGGACATTGCCCTGATGGAGCTGGAC | | |
| | TCCCCCTTGCAGGCCTCCCAGTTCAGCCCCATCTGCCTCCCAGGACCCCAGACCCCCCTCGCCATTG | | |
| | GGACCGTGTGCTGGGTAAACGGGCTGGGGCCCACATCACATCCAGCCCTGGCGAGTGTCCTTCAGGA | | |
| | GGTGGCTGTGCCCCTCCTGGACTCGAACATGTGTGAGCTGATGTACCACCTAGGAGAGCCCAGCCTG | | |
| | GCTGGCCAGCGCCTCATCCAGGACGACATGCTCTGTGCTGGCTCTGTCCAGGGCAAGAAAGACTCCT | | |
| | GCCAGGGTGACTCCGGGGGGCCGCTGGTCTGCCCCATCAATGATACGTGGATCCAGGCCGGCATTGT | | |
| | GAGCTGGGGATTCGGCTGTGCCCGGCCTTTCCGGCCTGGTGTCTACACCCAGGTGCTAAGCTACACA | | |
| | GACTGGATTCAGAGAACCCTGGCTGAATCTCACTCAGGCATGTCTGGGGCCCGCCCAGGTGCCCCAG | | |
| | GATCCCACTCAGGCACCTCCAGATCCCACCCAGTGCTGCTGCTTGAGCTGTTGACCGTATGCTTGCT | | |
| | TGGGTCCCTGTGA | | |
| | ORF Start: ATG at 1 | | ORF Stop: TGA at 949 |
| | SEQ ID NO: 118 | 316 aa | MW at 33574.2 kD |

| | |
|---|---|
| NOV25a, | MGLRAGPILLLLLWLLPGAHWDVLPSECGHSKEAGRIVGGQDTQEGRWPWQVGLWLTSVGHVCGGSL |
| CG147274-01 | |
| Protein Sequence | IHPRWVLTAAHCFLRSEDPGLYHVKVGGLTPSLSEPHSALVAVRRLLVHSSYHGTTTSGDIALMELD |
| | SPLQASQFSPICLPGPQTPLAIGTVCWVNGLGPTSHPALASVLQEVAVPLLDSNMCELMYHLGEPSL |
| | AGQRLIQDDMLCAGSVQGKKDSCQGDSGGPLVCPINDTWIQAGIVSWGFGCARPFRPGVYTQVLSYT |
| | DWIQRTLAESHSGMSGARPGAPGSHSGTSRSHPVLLLELLTVCLLGSL |

Further analysis of the NOV25a protein yielded the following properties shown in Table 25B.

TABLE 25B
Protein Sequence Properties NOV25a

| | |
|---|---|
| PSort analysis: | 0.9190 probability located in plasma membrane; |
| | 0.3000 probability located in lysosome (membrane); |
| | 0.1000 probability located in endoplasmic reticulum |
| | (membrane); 0.1000 probability located in |
| | endoplasmic reticulum (lumen) |
| SignalP analysis: | Cleavage site between residues 22 and 23 |

A search of the NOV25a protein against the Geneseq database, a proprietary database that contains sequences published in patents and patent publication, yielded several homologous proteins shown in Table 25C.

TABLE 25C
Geneseq Results for NOV25a

| | | NOV25a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAU98887 | Human protease PRTS5 - | 1 . . . 316 | 304/316 (96%) | 0.0 |
| | Homo sapiens, 304 aa. | 1 . . . 304 | 304/316 (96%) | |
| | [WO200238744-A2, | | | |
| | 16-MAY-2002] | | | |
| AAW77303 | Amino acid sequence of | 28 . . . 316 | 285/289 (98%) | e−171 |
| | SP002LA, a homologue of | 1 . . . 289 | 285/289 (98%) | |
| | HELA2 - Homo sapiens, 289 | | | |
| | aa. [WO9836054-A1, | | | |
| | 20-AUG-1998] | | | |
| ABG64545 | Human albumin fusion | 5 . . . 275 | 121/275 (44%) | 1e−63 |
| | protein #1220 - Homo | 6 . . . 276 | 168/275 (61%) | |
| | sapiens, 290 aa. | | | |
| | [WO200177137-A1, | | | |
| | 18-OCT-2001] | | | |
| AAB73945 | Human protease T - Homo | 5 . . . 275 | 121/275 (44%) | 1e−63 |
| | sapiens, 290 aa. | 6 . . . 276 | 168/275 (61%) | |
| | [WO200116293-A2, | | | |
| | 08-MAR-2001] | | | |
| AAE03821 | Human gene 4 encoded | 5 . . . 275 | 121/275 (44%) | 1e−63 |
| | secreted protein HWHIH10, | 6 . . . 276 | 168/275 (61%) | |
| | SEQ ID NO: 67 - Homo sapiens, | | | |
| | 290 aa. | | | |
| | [WO200136440-A1, | | | |
| | 25-MAY-2001] | | | |

In a BLAST search of public sequence datbases, the NOV25a protein was found to have homology to the proteins shown in the BLASTP data in Table 25D.

TABLE 25D
Public BLASTP Results for NOV25a

| | | NOV25a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q91XC4 | Similar to distal intestinal | 1 . . . 316 | 202/317 (63%) | e−114 |
| | serine protease - Mus | 1 . . . 310 | 235/317 (73%) | |
| | musculus (Mouse), 310 aa. | | | |
| Q9QYZ9 | Distal intestinal serine | 1 . . . 316 | 201/317 (63%) | e−113 |
| | protease - Mus musculus | 1 . . . 310 | 233/317 (73%) | |
| | (Mouse), 310 aa. | | | |
| Q9BQR3 | Marapsin precursor (EC | 5 . . . 275 | 121/275 (44%) | 3e−63 |
| | 3.4.21.−) - Homo sapiens | 6 . . . 276 | 168/275 (61%) | |
| | (Human), 290 aa. | | | |
| Q8R1A6 | RHKIEN cDNA 2010001P08 | 24 . . . 305 | 142/293 (48%) | 5e−62 |
| | gene - Mus musculus | 41 . . . 329 | 174/293 (58%) | |
| | (Mouse), 331 aa. | | | |
| Q9DGR3 | Embryonic serine protease-1 - | 25 . . . 304 | 123/288 (42%) | 1e−59 |
| | Xenopus laevis (African | 29 . . . 308 | 165/288 (56%) | |
| | clawed frog), 317 aa. | | | |

PFam analysis predicts that the NOV25a protein contains the domains shown in the Table 25E.

Domain Analysis of NOV25a

| | | Identities/ | |
|---|---|---|---|
| | NOV25a | Similarities for | |
| Pfam Domain | Match Region | the Matched Region | Expect Value |
| trypsin | 37 . . . 271 | 109/266 (41%) | 1.7e−79 |
| | | 176/266 (66%) | |

### Example 26

The NOV26 clone was analyzed, and the nucleotide and encoded polypeptide sequences are shown in Table 26A.

TABLE 26A
NOV26 Sequence Analysis

| | SEQ ID NO: 119 | 970 bp | |
|---|---|---|---|
| NOV26a, | CACAAGAACAATATGCAGCTGAGATGAGTAAAGCTATTGCTTTTGAGATCATTCAGAAATACGAGCC | | |
| CG147351-01 | | | |
| DNA Sequence | TATCGAAGAAGTTAGGAAAGCACACCAAATGTCATTAGAAGGTTTTACAAGATACATGGATTCACGT | | |
| | GAATGTCTACTGTTTAAAAATGAATGTAGAAAAGTTTATCAAGATATGACTCATCCATTAAATGATT | | |
| | ATTTTATTTCATCTTCACATAACACATATTTGGTATCTGATCAATTATTGGGACCAAGTGACCTTTG | | |
| | GGGATATGTAAGTGCCCTTGTGAAAGGATGCCGTTGTTTGGAGATTGACTGCTGGGATGGAGCACAA | | |
| | AATGAACCTGTTGTATATCATGGCTACACACTCACAAGCAAACTTCTGTTTAAAACTGTTATCCAAG | | |
| | CTATACACAAGTATGCATTCATGGTGGCTTTAAATTTCCAGACCCCTGGTCTGCCCATGGATCTGCA | | |
| | AAATGGGAAATTTTTGGATAATGGTGGTTCTGGATATATTTTGAAACCACATTTCTTAAGAGAGAGT | | |
| | AAATCATACTTTAACCCAAGTAACATAAAAGAGGGTATGCCAATTACACTTACAATAAGGCTCATCA | | |
| | GTGGTATCCAGTTGCCTCTTACTCATTCATCATCTAACAAAGGTGATTCATTAGTAATTATAGAAGT | | |
| | TTTTGGTGTTCCAAATGATCAAATGAAGCAGCAGACTCGTGTAATTAAAAAAAATGCTTTTAGTCCA | | |
| | AGATGGAATGAAACATTCACATTTATTATTCATGTCCCAGAATTGGCATTGATACGTTTTGTTGTTG | | |
| | AAGGTCAAGGTTTAATAGCAGGAAATGAATTTCTTGGGCAATATACTTTGCCACTTCTATGCATGAA | | |
| | CAAAGGTTATCGTCGTATTCCTCTGTTTTCCAGAATGGGTGAGAGCCTTGAGCCTGCTTCACTGTTT | | |
| | GTTTATGTTTGGTACGTCAGATAACAGCTAAG | | |
| | ORF Start: ATG at 24 | | ORF Stop: TAA at 960 |
| | SEQ ID NO: 120 | 312 aa | MW at 35720.0 kD |

| | |
|---|---|
| NOV26a, | MSKAIAFEIIQKYEPIEEVRKAHQMSLEGFTRYMDSRECLLFKNECRKVYQDMTHPLNDYFISSSHN |
| CG147351-01 | |
| Protein Sequence | TYLVSDQLLGPSDLWGYVSALVKGCRCLEIDCWDGAQNEPVVYHGYTLTSKLLFKTVIQAIHKYAFM |
| | VALNFQTPGLPMDLQNGKFLDNGGSGYILKPHFLRESKSYFNPSNIKEGMPITLTIRLISGIQLPLT |
| | HSSSNKGDSLVIIEVFGVPNDQMKQQTRVIKKNAFSPRWNETFTFIIHVPELALIRFVVEGQGLIAG |
| | NEFLGQYTLPLLCMNKGYRRIPLFSRMGESLEPASLFVYVWYVR |

Further analysis of the NOV26a protein yielded the following properties shown in Table 26B.

TABLE 26B
Protein Sequence Properties NOV26a

| | |
|---|---|
| PSort | 0.5844 probability located in microbody (peroxisome); |
| analysis: | 0.1814 probability located in lysosome (lumen); 0.1000 |
| | probability located in mitochondrial matrix space; 0.0000 |
| | probability located in endoplasmic reticulum (membrane) |
| SignalP | No Known Signal Sequence Predicted |
| analysis: | |

A search of the NOV26a protein against the Geneseq database, a proprietary database that contains sequences published in patents and patent publication, yielded several homologous proteins shown in Table 26C.

TABLE 26C
Geneseq Results for NOV26a

| | | NOV26a | | |
|---|---|---|---|---|
| | | Residues/ | Identities/ | |
| Geneseq | Protein/Organism/Length | Match | Similarities for the | Expect |
| Identifier | [Patent #, Date] | Residues | Matched Region | Value |
| AAU76817 | Human phospholipase C | 134 . . . .312 | 179/179 (100%) | e−101 |
| | 16839 polypeptide - Homo | 430 . . . 608 | 179/179 (100%) | |
| | sapiens, 608 aa. | | | |
| | [WO200206302-A2, | | | |
| | 24-JAN-2002] | | | |
| ABB90425 | Human polypeptide SEQ ID | 134 . . . 312 | 179/179 (100%) | e−101 |
| | NO 2801 - Homo sapiens, | 1 . . . 179 | 179/179 (100%) | |
| | 179 aa. [WO200190304-A2, | | | |
| | 29-NOV-2001] | | | |
| AAU87271 | Novel central nervous | 134 . . . 312 | 179/179 (100%) | e−101 |
| | system protein #181 - Homo | 76 . . . 254 | 179/179 (100%) | |
| | sapiens, 254 aa. | | | |
| | [WO200155318-A2, | | | |
| | 02-AUG-2001] | | | |
| AAM95867 | Human reproductive system | 134 . . . 312 | 178/179 (99%) | e−100 |
| | related antigen SEQ ID NO: | 76 . . . 254 | 178/179 (99%) | |
| | 4525 - Homo sapiens, 254 | | | |
| | aa. [WO200155320-A2, | | | |
| | 02-AUG-2001] | | | |
| AAU22938 | Novel human enzyme | 134 . . . .312 | 178/179 (99%) | e−100 |
| | polypeptide #24 - Homo | 76 . . . 254 | 178/179 (99%) | |
| | sapiens, 254 aa. | | | |
| | [WO200155301-A2, | | | |
| | 02-AUG-20011 | | | |

In a BLAST search of public sequence datbases, the NOV26a protein was found to have homology to the proteins shown in the BLASTP data in Table 26D.

TABLE 26D
Public BLASTP Results for NOV26a

| | | NOV26a | Identities/ | |
|---|---|---|---|---|
| Protein | | Residues/ | Similarities for | |
| Accession | | Match | the Matched | Expect |
| Number | Protein/Organism/Length | Residues | Portion | Value |
| BAC05152 | CDNA FLJ40406 fis, clone | 134 . . . 312 | 179/179(100%) | e−101 |
| | TESTI2037534, weakly similar to | 212 . . . 390 | 179/179(100%) | |
| | 1-PHOSPHATIDYLINOSITOL−4,5−B | | | |
| | ISPHOSPHATE | | | |
| | PHOSPHODIESTERASE DELTA 1 | | | |
| | (EC 3.1.4.11) - Homo sapiens | | | |
| | (Human), 390 aa. | | | |
| Q96J70 | Testis-development related NYD-SP27 - | 134 . . . 312 | 178/179 (99%) | e−100 |
| | Homo sapiens (Human), 504 aa. | 326 . . . 504 | 178/179 (99%) | |
| Q95JSO | Hypothetical 74.4 kDa protein - | 134 . . . 312 | 172/179 (96%) | 2e−97 |
| | Macaca fascicularis (Crab eating | 462 . . . 640 | 177/179 (98%) | |
| | macaque)(Cynomolgus monkey), 640 | | | |
| | aa. | | | |
| Q95JS1 | Hypothetical 74.6 kDa protein - | 134 . . . 312 | 172/179 (96%) | 2e−97 |
| | Macaca fascicularis (Crab eating | 463 . . . 641 | 177/179 (98%) | |
| | macaque)(Cynomolgus monkey), 641 | | | |
| | aa. | | | |
| AAM95914 | PLC-zeta - Mus musculus (Mouse), | 134 . . . 312 | 135/181 (74%) | 7e−73 |
| | 647 aa. | 467 . . . 646 | 158/181 (86%) | |

PFam analysis predicts that the NOV26a protein contains the domains shown in the Table 26E.

TABLE 26E
Domain Analysis of NOV26a

| | | Identities/ | |
|---|---|---|---|
| | | Similarities | |
| | NOV26a | for the Matched | |
| Pfam Domain | Match Region | Region | Expect Value |
| PI-PLC-X | 52 . . . 133 | 45/83 (54%) | 4.3e−36 |
| | | 66/83 (80%) | |
| PI-PLC-Y | 134 . . . 169 | 25/43 (58%) | 2.9e−17 |
| | | 33/43 (77%) | |
| C2 | 188 . . . 276 | 33/97 (34%) | 4.9e−20 |
| | | 73/97 (75%) | |

### Example 27

The NOV27 clone was analyzed, and the nucleotide and encoded polypeptide sequences are shown in Table 27A.

TABLE 27A
NOV27 Sequence Analysis

| | SEQ ID NO: 121 | 3136 bp | |
|---|---|---|---|
| NOV27a, | AGGGAGTCGTGTCGGCGCCACCCCGGCCCCCGAGCCCGCAGATTGCCCACCGAAGCTCGTGTGTGCA | | |
| CG147419-01 | | | |
| DNA Sequence | CCCCCGATCCCGCCAGCCACTCGCCCCTGGCCTCGCGGGCCGTGTCTCCGGCATCATGTGTGGTATA | | |
| | TTTGCTTACTTAAACTACCATGTTCCTCGAACGAGACGAGAAATCCTGGAGACCCTAATCAAAGGCC | | |
| | TTCAGAGACTGGAGTACAGAGGATATGATTCTGCTGGTGTGGGATTTGATGGAGGCAATGATAAAGA | | |
| | TTGGGAAGCCAATGCCTGCAAAACCCAGCTTATTAAGAAGAAAGGAAAAGTTAAGGCACTGGATGAA | | |
| | GAAGTTCACAAGCAACAAGATATGGATTTGGATATAGAATTTGATGTACACCTTGGAATAGCTCATA | | |
| | CCCGTTGGGCAACACATGGAGAACCCAGTCCTGTCAATAGCCACCCCCAGCGCTCTGATAAAAATAA | | |
| | TGAATTTATCGTTATTCACAATGGCATCATCACCAACTACAAAGACTTGAAAAAGTTTTTGGAAAGC | | |
| | AAAGGCTATGACTTCGAATCTGAAACAGACACAGAGACAATTGCCAAGCTCGTTAAGTATATGTATG | | |
| | ACAATCGGGAAAGTCAAGATACCAGCTTTACTACCTTGGTGGAGAGAGTTATCCAACAATTGGAAGG | | |
| | TGCTTTTGCACTTGTGTTTAAAAGTGTTCATTTTCCCGGGCAAGCAGTTGGCACAAGGCGAGGTAGC | | |
| | CCTCTGTTGATTGGTGTACGGAGTGAACATAAACTTTCTACTGATCACATTCCTATACTCTACAGAA | | |
| | CAGCTAGGACTCAGATTGGATCAAAATTCACACGGTGGGGATCACAGGGAGAAAGAGGCAAAGACAA | | |
| | GAAAGGAAGCTGCAATCTCTCTCGTGTGGACAGCACAACCTGCCTTTTCCCGGTGGAAGAAAAAGCA | | |
| | GTGGAGTATTACTTTGCTTCTGATGCAAGTGCTGTCATAGAACACACCAATCGCGTCATCTTTCTGG | | |
| | AAGATGATGATGTTGCAGCAGTAGTGGATGGACGTCTTTCTATCCATCGAATTAAACGAACTGCAGG | | |
| | AGATCACCCCGGACGAGCTGTGCAAACACTCCAGATGGAACTCCAGCAGATCATGAAGGGCAACTTC | | |
| | AGTTCATTTATGCAGAAGGAAATATTTGAGCAGCCAGAGTCTGTCGTGAACACAATGAGAGGAAGAG | | |
| | TCAACTTTGATGACTATACTGTGAATTTGGGTGGTTTGAAGGATCACATAAAGGAGATCCAGAGATG | | |
| | CCGGCGTTTGATTCTTATTGCTTGTGGAACAAGTTACCATGCTGGTGTAGCAACACGTCAAGTTCTT | | |
| | GAGGAGCTGACTGAGTTGCCTGTGATGGTGGAACTAGCAAGTGACTTCCTGGACAGAAACACACCAG | | |
| | TCTTTCGAGATGATGTTTGCTTTTTCCTTAGTCAATCAGGTGAGACAGCAGATACTTTGATGGGTCT | | |
| | TCGTTACTGTAAGGAGAGAGGAGCTTTAACTGTGGGGATCACAAACACAGTTGGCAGTTCCATATCA | | |
| | CGGGAGACAGATTGTGGAGTTCATATTAATGCTGGTCCTGAGATTGGTGTGGCCAGTACAAAGGCTT | | |
| | ATACCAGCCAGTTTGTATCCCTTGTGATGTTTGCCCTTATGATGTGTGATGATCGGATCTCCATGCA | | |
| | AGAAAGACGCAAAGAGATCATGCTTGGATTGAAACGGCTGCCTGATTTGATTAAGGAAGTACTGAGC | | |
| | ATGGATGACGAAATTCAGAAACTAGCAACAGAACTTTATCATCAGAAGTCAGTTCTGATAATGGGAC | | |
| | GAGGCTATCATTATGCTACTTGTCTTGAAGGGGCACTGAAAATCAAAGAAATTACTTATATGCACTC | | |
| | TGAAGGCATCCTTGCTGGTGAATTGAAACATGGCCCTCTGGCTTTGGTGGATAAATTGATGCCTGTG | | |
| | ATCATGATCATCATGAGAGATCACACTTATGCCAAGTGTCAGAATGCTCTTCAGCAAGTGGTTGCTC | | |
| | GGCAGGGGCGGCCTGTGGTAATTTGTGATAAGGAGGATACTGAGACCATTAAGAACACAAAAAGAAC | | |
| | GATCAAGGTGCCCCACTCAGTGGACTGCTTGCAGGGCATTCTCAGCGTGATCCCTTTACAGTTGCTG | | |
| | GCTTTCCACCTTGCTGTGCTGAGAGGCTATGATGTTGATTTCCCACGGAATCTTGCCAAATCTGTGA | | |
| | CTGTAGAGTGAGGAATATCTATACAAAATGTACGAAACTGTATGATTAAGCAACACAAGACACCTTT | | |
| | TGTATTTAAAACCTTGATTTAAAATATCACCCCTTGAAGCCTTTTTTTAGTAAATCCTTATTTATAT | | |
| | ATCAGTTATAATTATTCCACTCAATATGTGATTTTTGTGAAGTTACCTCTTACATTTTCCCAGTAAT | | |
| | TTGTGGAGGACTTTGAATAATGGAATCTATATTGGAATCTGTATCAGAAAGATTCTAGCTATTATTT | | |
| | TCTTTAAAGAATGCTGGGTGTTGCATTTCTGGACCCTCCACTTCAATCTGAGAAGACAATATGTTTC | | |
| | TAAAAATTGGTACTTGTTTCACCATACTTCATTCAGACCAGTGAAAGAGTAGTGCATTTAATTGGAG | | |
| | TATCTAAAGCCAGTGGCAGTGTATGCTCATACTTGGACAGTTAGGGAAGGGTTTGCCAAGTTTTAAG | | |
| | AGAAGATGTGATTTATTTTGAAATTTGTTTCTGTTTTGTTTTTAAATCAAACTGTAAAACTTAAAAC | | |
| | TGAAAAATTTTATTGGTAGGATTTATATCTAAGTTTGGTTAGCCTTAGTTTCTCAGACTTGTTGTCT | | |
| | ATTATCTGTAGGTGGAAGAAATTTAGGAAGCGAAATATTACAGTAGTGCATTGGTGGGTCTCAATCC | | |
| | TTAACATATTTGCACAATTTTATAGCACAAACTTTAAATTCAAGCTGCTTTGGACAACTGACAATAT | | |
| | GATTTTAAATTTGAAGATGGGATGTGTACATGTTGGGTATCCTACTACTTTGTGTTTTCATCTCCTA | | |
| | AAAGTGTTTTTTATTTCCTTGTATCTGTAGTCTTTTATTTTTTAAATGACTGCTGAATGACATATTT | | |
| | TATCTTGTTCTTTAAAATCACAACACAGAGCTGCTATTAAATTAATATTGATAT | | |
| | ORF Start: ATG at 123 | | ORF Stop: TGA at 2220 |
| | SEQ ID NO: 122 | 699 aa | MW at 78793.6 kD |

| | |
|---|---|
| NOV27a, | MCGIFAYLNYHVPRTRREILETLIKGLQRLEYRGYDSAGVGFDGGNDKDWEANACKTQLIKKKGKVK |
| CG147419-01 | |
| Protein Sequence | ALDEEVHKQQDMDLDIEFDVHLGIAHTRWATHGEPSPVNSHPQRSDKNNEFIVIHNGIITNYKDLKK |
| | FLESKGYDFESETDTETIAKLVKYMYDNRESQDTSFTTLVERVIQQLEGAFALVFKSVHFPGQAVGT |
| | RRGSPLLIGVRSEHKLSTDHIPILYRTARTQIGSKFTRWGSQGERGKDKKGSCNLSRVDSTTCLFPV |
| | EEKAVEYYFASDASAVIEHTNRVIFLEDDDVAAVVDGRLSIHRIKRTAGDHPGRAVQTLQMELQQIM |
| | KGNFSSFMQKEIFEQPESVVNTMRGRVNFDDYTVNLGGLKDHIKEIQRCRRLILIACGTSYHAGVAT |
| | RQVLEELTELPVMVELASDFLDRNTPVFRDDVCFFLSQSGETADTLMGLRYCKERGALTVGITNTVG |
| | SSISRETDCGVHINAGPEIGVASTKAYTSQFVSLVMFALMMCDDRISMQERRKEIMLGLKRLPDLIK |
| | EVLSMDDEIQKLATELYHQKSVLIMGRGYHYATCLEGALKIKEITYMHSEGILAGELKHGPLALVDK |
| | LMPVIMIIMRDHTYAKCQNALQQVVARQGRPVVICDKEDTETIKNTKRTIKVPHSVDCLQGILSVIP |
| | LQLLAFHLAVLRGYDVDFPRNLAKSVTVE |

Further analysis of the NOV27a protein yielded the following properties shown in Table 27B.

TABLE 27B
Protein Sequence Properties NOV27a

| | |
|---|---|
| PSort | 0.4902 probability located in mitochondrial inner membrane; |
| analysis: | 0.4400 probability located in plasma membrane; |
| | 0.3000 probability located in |
| | microbody (peroxisome); 0.2000 probability located in |
| | endoplasmic reticulum (membrane) |
| SignalP | No Known Signal Sequence Predicted |
| analysis: | |

A search of the NOV27a protein against the Geneseq database, a proprietary database that contains sequences published in patents and patent publication, yielded several homologous proteins shown in Table 27C.

TABLE 27C
Geneseq Results for NOV27a

| | | NOV27a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB05747 | Human GFAT1L protein | 1 . . . 699 | 698/699 (99%) | 0.0 |
| | SEQ ID NO: 1 - Homo | 1 . . . 699 | 698/699 (99%) | |
| | sapiens, 699 aa. | | | |
| | [WO200196574-A1, | | | |
| | 20-DEC-2001] | | | |
| AAY90260 | Human GFAT protein | 1 . . . 681 | 681/699 (97%) | 0.0 |
| | sequence - Homo sapiens, | 1 . . . 681 | 681/699 (97%) | |
| | 681 aa. [WO200037617-AI, | | | |
| | 29-JUN-2000] | | | |
| AAR43348 | Human GFAT - Homo | 1 . . . 699 | 680/699 (97%) | 0.0 |
| | sapiens, 681 aa. | 1 . . . 681 | 680/699 (97%) | |
| | [WO9321330-A, | | | |
| | 28-OCT-1993] | | | |
| AAY90261 | Human GFAT II protein | 1 . . . 699 | 541/701 (77%) | 0.0 |
| | sequence - Homo sapiens, | 1 . . . 682 | 618/701 (87%) | |
| | 682 aa. [WO200037617-Al, | | | |
| | 29-JUN-2000] | | | |
| AW37772 | Huma | 1 . . . 699 | 541/701 (77%) | 0.0 |
| | glutamine: fructose-6- | 1 . . . 682 | 618/701 (87%) | |
| | phosphate amidotransferase | | | |
| | TGC028−4 - Homo sapiens, | | | |
| | 682 aa. [EP824149−A2, | | | |
| | 18-FEB-1998] | | | |

In a BLAST search, of public sequence datbases, the NOV27a protein was found to have homology to the proteins shown in the BLASTP data in Table 27D.

TABLE 27D
Public BLASTP Results for NOV27a

| | | NOV27a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q99MJ4 | Glutamine: fructose-6-phosphate | 1 . . . 699 | 688/699 (98%) | 0.0 |
| | amidotransferase 1 muscle | 1 . . . 697 | 690/699 (98%) | |
| | isoform GFAT1M - Mus | | | |
| | musculus (Mouse), 697 aa. | | | |
| A45055 | glutamine--fructose-6-phosphatae | 1. . . 699 | 681/699 (97%) | 0.0 |
| | transaminase (isomerizing)(EC | 1 . . . 681 | 681/699 (97%) | |
| | 2.6.1.16) - human, 681 aa. | | | |
| Q06210 | Glucosamine--fructose-6-phospha | 2 . . . 699 | 680/698 (97%) | 0.0 |
| | te aminotransferase [isomerizing] | 1. . . 680 | 680/698 (97%) | |
| | 1 (EC 2.6.1.16)(Hexosephosphate | | | |
| | aminotransferase 1) | | | |
| | (D-fructose-6- phosphate | | | |
| | amidotransferase 1)(GFAT 1) | | | |
| | (GFAT1) - Homo sapiens | | | |
| | (Human), 680 aa. | | | |
| BAB31882 | Gfptl protein - Mus musculus | 1 . . . 699 | 674/699 (96%) | 0.0 |
| | (Mouse), 681 aa. | 1 . . . 681 | 676/699 (96%) | |
| P47856 | Glucosamine--fructose-6-phosphate | 2 . . . 699 | 673/698 (96%) | 0.0 |
| | aminotransferase [isomerizing] | 1 . . . 680 | 675/698 (96%) | |
| | 1 (EC 2.6.1.16)(Hexosephosphate | | | |
| | aminotransferase 1) | | | |
| | (D-fructose-6- phosphate | | | |
| | amidotransferase 1)(GFAT 1) | | | |
| | (GFAT1) - Mus musculus | | | |
| | (Mouse), 680 aa. | | | |

PFam analysis predicts that the NOV27a protein contains the domains shown in the Table 27E.

TABLE 27E
Domain Analysis of NOV27a

| | | Identities/ | |
|---|---|---|---|
| | | Similarities | |
| | NOV27a | for the Matched | |
| Pfam Domain | Match Region | Region | Expect Value |
| GATase_2 | 2 . . . 210 | 91/219(42%) | 4.6e−127 |
| | | 202/219 (92%) | |
| SIS | 378 . . . 512 | 52/156 (33%) | 2.2e−48 |
| | | 118/156 (76%) | |
| SIS | 549 . . . 685 | 52/156 (33%) | 3.3e−46 |
| | | 124/156 (79%) | |

### Example 28

The NOV28 clone was analyzed, and the nucleotide and encoded polypeptide sequences are shown in Table 28A.

TABLE 28A
NOV28 Sequence Analysis

| | SEQ ID NO: 123 | 2521 bp | |
|---|---|---|---|
| NOV28a, | ACTCTGCCCGACTCAGGGCTCCAGCGTGACATGGCTGAAGCGCACCAGGCCGTGGGCTTCCGACCCT | | |
| CG148102-01 | | | |
| DNA Sequence | CGCTGACCTCGGACGGGGCTGAAGTGGAACTCAGTGCCCCTGTGCTGCAGGAGATCTACCTCTCTGG | | |
| | CCTGCGCTCCTGGAAAAGGCATCTCTCACGTTTCTGGGTGCAGAATGACTTTCTCACCGGTGTGTTT | | |
| | CCTGCCAGCCCCCTCAGTTGGCTTTTCCTCTTCAGTGCCATCCAGCTTGCCTGGTTCCTCCAGCTGG | | |
| | ATCCTTCCTTAGGACTGATGGAGAAGATCAAAGAGTTGCTGCGGGGGGTCCTGGCAGCCGCGCTGTT | | |
| | TGCCTCGTGTTTGTGGGGAGCCCTGATCTTCACACTGCACGTGGCCCTGAGGCTGCTTCTGTCCTAC | | |
| | CACGGCTGGCTTCTTGAGCCCCACGGAGCCATGTCCTCCCCCACCAAGACCTGGCTGGCCCTGGTCC | | |
| | GCATCTTCTCTGGCCGCCACCCGATGCTGTTCAGTTACCAGCGCTCCCTGCCACGCCAGCCCGTGCC | | |
| | CTCTGTGCAGGACACCGTGCGCAAGTACCTGGAGTCGGTCCGGCCCATCCTCTCCGACGAGGACTTC | | |
| | GACTGGACCGCGGTCCTGGCGCAGGAATTCCTGAGGCTGCAGGCGTCGCTGCTGCAGTGGTACCTGC | | |
| | GGCTCAAGTCCTGGTGGGCGTCCAATTATGTGAGTGACTGGTGGGAGGAATTTGTGTACCTGCGCTC | | |
| | CCGAAATCCGCTGATGGTGAACAGCAACTATTACATGATGGACTTCCTGTATGTCACACCCACGCCT | | |
| | CTGCAGGCAGCTCGCGCTGGGAATGCCGTCCATGCCCTCCTCCTGTACCGCCACCGCCTGAACCGCC | | |
| | AGGAGATACCCCCGGTGAGACTGATGGGAATGCGCCCCTTATGCTCTGCCCAGTACGAGAAGATCTT | | |
| | CAACACCACGCGGATTCCAGGGGTCCAAAAAGGTGAGACCATCCGCCACCTCCATGACAGCCAACAC | | |
| | GTGGCTGTCTTCCACCGGGGCCGATTCTTCCGCATGGGGACCCACTCCCGAAACAGCCTGCTTTCCC | | |
| | CGAGAGCCCTGGAGCAGCAGTTTCAGAGAATCCTGGATGATCCCTCACCGGCCTGCCCCCACGAGGA | | |
| | ACATCTGGCAGCTCTGACAGCTGCTCCCAGGGGCACGTGGGCCCAGGTGCGGACATCCCTGAAGACC | | |
| | CAGGCAGCGGAGGCCCTGGAGGCGGTGGAAGGGGCCGCTTTCTTTGTGTCACTGGATGCTGAGCCCG | | |
| | CGGGGCTCACCAGGGAGGACCCGGCAGCGTCGTTGGATGCCTACGCCCATGCTCTGCTGGCCGGCCG | | |
| | GGGCCATGATCGGTGGTTTGACAAATCCTTCACCCTAATCGTCTTCTCTAACGGGAAGCTGGGCCTC | | |
| | AGCGTGGAGCACTCCTGGGCCGACTGCCCCATCTCAGGACACATGTGGGAGTTCACTCTGGCTACAG | | |
| | AATGCTTTCAGCTGGGCTACTCAACAGACGGCCACTGCAAGGGGCACCCGGACCCCACACTACCCCA | | |
| | GCCCCAGCGGCTGCAATGGGACCTTCCAGACCAGGTGAGGCTGGGTATCTCTCTAGCCCTGAGGGGA | | |
| | GCCAAGATCTTGTCTGAAAATGTCGACTGCCATGTCGTTCCATTCTCCCTATTTGGCAAGAGCTTCA | | |
| | TCCGACGCTGCCACCTCTCTTCAGACAGCTTCATCCAGATCGCCTTGCAACTGGCCCACTTCCGGGA | | |
| | CCCACAGTGCCTCGCCCTGTTCCGCGTGGCAGTGGACAAGCACCAGGCTCTGCTGAAGGCAGCCATG | | |
| | AGCGGGCAGGGAGTTGACCGCCACCTGTTTGCGCTGTACATCGTGTCCCGATTCCTCCACCTGCAGT | | |
| | CGCCCTTCCTGACCCAGGTCCATTCGGAGCAGTGGCAGCTGTCCACCAGCCAGATCCCTGTTCAGCA | | |
| | AATGCATCTGTTTGACGTCCACAATTACCCGGACTATGTTTCCTCAGGCGGTGGATTCGGGCCTGCT | | |
| | GATGACCATGGTTATGGTGTTTCTTATATCTTCATGGGGGATGGCATGATCACCTTCCACATCTCCA | | |
| | GCAAAAAATCAAGCACAAAAACGGATTCCCACAGGCTGGGGCAGCACATTGAGGACGCACTGCTGGA | | |
| | TGTGGCCTCCCTGTTCCAGGCGGGACAGCATTTTAAGCGCCGGTTCAGAGGGTCAGGGAAGGAGAAC | | |
| | TCCAGGCACAGGTGTGGATTTCTCTCCCGCCAGACTGGGGCCTCCAAGGCCTCAATGACATCCACCG | | |
| | ACTTCTGACTCCTTCCAGCAGGCAGCTGGCCTCTCCAAGGAATAAGGGTGAAATTGCCACAGCTGGC | | |
| | TGACACAGGACAGGGGCAACTGGTTTGGCAACCCCACATCCAGGCCAATAAAGATGTGTGAGCTGGG | | |
| | TGTGTGGTGTCTGCTATGCTCTTGGGCAGGGCAGGGGTAGAAGAGGTAAGGACCAGGGTGGAGGAGG | | |
| | ACAGAAGCTCCCATCCATTCCCAGGCCCAGCCAGGGATTCCC | | |
| | ORF Start: ATG at 31 | | ORF Stop: TGA at 2284 |
| | SEQ ID NO: 124 | 751 aa | MW at 84918.2 kD |
| NOV28a, | MAEAHQAVGFRPSLTSDGAEVELSAPVLQEIYLSGLRSWKRHLSRFWVQNDFLTGVFPASPLSWLFL | | |
| CG148102-01 | | | |
| Protein Sequence | FSAIQLAWFLQLDPSLGLMEKIKELLRGVLAAALFASCLWGALIFTLHVALRLLLSYHGWLLEPHGA | | |
| | MSSPTKTWLALVRIFSGRHPMLFSYQRSLPRQPVPSVQDTVRKYLESVRPILSDEDFDWTAVLAQEF | | |
| | LRLQASLLQWYLRLKSWWASNYVSDWWEEFVYLRSRNPLMVNSNYYMMDFLYVTPTPLQAARAGNAV | | |
| | HALLLYRHRLNRQEIPPVRLMGMRPLCSAQYEKIFNTTRIPGVQKGETIRHLHDSQHVAVFHRGRFF | | |
| | RMGTHSRNSLLSPRALEQQFQRILDDPSPACPHEEHLAALTAAPRGTWAQVRTSLKTQAAEALEAVE | | |
| | GAAFFVSLDAEPAGLTREDPAASLDAYAHALLAGRGHDRWFDKSFTLIVFSNGKLGLSVEHSWADCP | | |
| | ISGHMWEFTLATECFQLGYSTDGHCKGHPDPTLPQPQRLQWDLPDQVRLGISLALRGAKILSENVDC | | |
| | HVVPFSLFGKSFIRRCHLSSDSFIQIALQLAHFRDPQCLALFRVAVDKHQALLKAAMSGQGVDRHLF | | |
| | ALYIVSRFLHLQSPFLTQVHSEQWQLSTSQIPVQQMHLFDVHNYPDYVSSGGGFGPADDHGYGVSYI | | |
| | FMGDGMITFHISSKKSSTKTDSHRLGQHIEDALLDVASLFQAGQHFKRRFRGSGKENSRHRCGFLSR | | |
| | QTGASKASMTSTDF | | |
| | SEQ ID NO: 125 | 2748 bp | |
| NOV28b, | CGAGAGACAGGAATCGGGGTTTCTGGGTGACGGTGATCTCGGGGTGGGCAGGACTCCAAAGGCCCGT | | |
| CG148102-02 | | | |
| DNA Sequence | CGACCCGGTGGTGGACTCCTTGCACTGGGATTGGACATATGCAAGCGGGAGATTTGGGGCCGGCGCT | | |
| | CAAAATCGGGGGGCGGGGGTGGACTCGGGTTTGGACCCCAGGATCCGATCAGCGGACCCTTGATTCA | | |
| | ACGTGGGCTCCAGCGTGACATGGCTGAAGCGCACCAGGCCGTGGGCTTCCGACCCTCGCTGACCTCG | | |
| | GACGGGGCTGAAGTGGAACTCAGTGCCCCTGTGCTGCAGGAGATCTACCTCTCTGGCCTGCGCTCCT | | |
| | GGAAAAGGCATCTCTCACGTTTCTGGAATGACTTTCTCACCGGTGTGTTTCCTGCCAGCCCCCTCAG | | |
| | TTGGCTTTTCCTCTTCAGTGCCATCCAGCTTGCCTGGTTCCTCCAGCTGGATCCTTCCTTAGGACTG | | |
| | ATGGAGAAGATCAAAGAGTTGCTGCCTGACTGGGGTGGACAACACCACGGGCTCCGGGGGGTCCTGG | | |
| | CAGCCGCGCTGTTTGCCTCGTGTTTGTGGGGAGCCCTGATCTTCACACTGCACGTGGCCCTGAGGCT | | |
| | GCTTCTGTCCTACCACGGCTGGCTTCTTGAGCCCCACGGAGCCATGTCCTCCCCCACCAAGACCTGG | | |
| | CTGGCCCTGGTCCGCATCTTCTCTGGCCGCCACCCGATGCTGTTCAGTTACCAGCGCTCCCTGCCAC | | |
| | GCCAGCCCGTGCCCTCTGTGCAGGACACCGTGCGCAAGTACCTGGAGTCGGTCCGGCCCATCCTCTC | | |
| | CGACGAGGACTTCGACTGGACCGCGGTCCTGGCGCAGGAATTCCTGAGGCTGCAGGCGTCACTGCTG | | |
| | CAGTGGTACCTGCGGCTCAAGTCCTGGTGGGCGTCCAATTATGTCAGTGACTGGTGGGAGGAATTTG | | |
| | TGTACCTGCGCTCCCGAAATCCGCTGATGGTGAACAGCAACTATTACATGATGGACTTCCTGTATGT | | |
| | CACACCCACGCCTCTGCAGGCAGCTCGCGCTGGGAATGCCGTCCATGCCCTCCTCCTGTACCGCCAC | | |
| | CGCCTGAACCGCCAGGAGATACCCCCGACTTTGCTGATGGGAATGCGCCCCTTATGCTCTGCCCAGT | | |
| | ACGAGAAGATCTTCAACACCACGCGGATTCCAGGGGTCCAAAAAGACTACATCCGCCACCTCCATGA | | |
| | CAGCCAACACGTGGCTGTCTTCCACCGGGGCCGATTCTTCCGCATGGGGACCCACTCCCGAAACAGC | | |
| | CTGCTTTCCCCGAGAGCCCTGGAGCAGCAGTTTCAGAGAATCCTGGATGATCCCTCACCGGCCTGCC | | |
| | CCCACGAGGAACATCTGGCAGCTCTGACAGCTGCTCCCAGGGGCACGTGGGCCCAGGTGCGGACATC | | |
| | CCTGAAGACCCAGGCAGCGGAGGCCCTGGAGGCGGTGGAAGGGGCCGCTTTCTTTGTGTCACTGGAT | | |
| | GCTGAGCCCGCGGGGCTCACCAGGGAGGACCCGGCAGCGTCGTTGGATGCCTACGCCCATGCTCTGC | | |
| | TGGCTGGCCGGGGCCATGATCGCTGGTTTGACAAATCCTTCACCCTAATCGTCTTCTCTAACGGGAA | | |
| | GCTGGGCCTCAGCGTGGAGCACTCCTGGGCCGACTGCCCCATCTCAGGACACATGTGGGAGTTCACT | | |
| | CTGGCTACAGAATGCTTTCAGCTGGGCTACTCAACAGATGGCCACTGCAAGGGGCACCCGGACCCCA | | |
| | CACTACCCCAGCCCCAGCGGCTGCAATGGGACCTTCCAGACCAGATCCACTCCTCCATCTCTCTAGC | | |
| | CCTGAGGGGAGCCAAGATCTTGTCTGAAAATGTCGACTGCCATGTCGTTCCATTCTCCCTATTTGGC | | |
| | AAGAGCTTCATCCGACGCTGCCACCTCTCTTCAGACAGCTTCATCCAGATCGCCTTGCAACTGGCCC | | |
| | ACTTCCGGGACAGGGGTCAATTCTGCCTGACTTATGAGTCGGCCATGACTCGCTTATTCCTGGAAGG | | |
| | CCGGACGGAGACGGTGCGGTCTTGCACGAGGGAGGCCTGCAACTTTGTCAGGGCCATGGAGGACAAA | | |
| | GAGAAGACGGACCCACAGTGCCTCGCCCTGTTCCGCGTGGCAGTGGACAAGCACCAGGCTCTGCTGA | | |
| | AGGCAGCCATGAGCGGGCAGGGAGTTGACCGCCACCTGTTTGCGCTGTACATCGTGTCCCGATTCCT | | |
| | CCACCTGCAGTCGCCCTTCCTGACCCAGGTCCATTCGGAGCAGTGGCAGCTGTCCACCAGCCAGATC | | |
| | CCTGTTCAGCAAATGCATCTGTTTGACGTCCACAATTACCCGGACTATGTTTCCTCAGGCGGTGGAT | | |
| | TCGGGCCTGCTGATGACCATGGTTATGGTGTTTCTTATATCTTCATGGGGGATGGCATGATCACCTT | | |
| | CCACATCTCCAGCAAAAAATCAAGCACAAAAACGGATTCCCACAGGCTGGGGCAGCACATTGAGGAC | | |
| | GCACTGCTGGATGTGGCCTCCCTGTTCCAGGCGGGACAGCATTTTAAGCGCCGGTTCAGAGGGTCAG | | |
| | GGAAGGAGAACTCCAGGCACAGGTGTGGATTTCTCTCCCGCCAGACTGGGGCCTCCAAGGCCTCAAT | | |
| | GACATCCACCGACTTCTGACTCCTTCCAGCAGGCAGCTGGCCTCTCCAAGGAATAAGGGTGAAATTG | | |
| | CCACAGCTGGCTGACACAGGACAGGGGCAACTGGTTTGGCAACCCCACATCCAGGCAAATAAAGATG | | |
| | G | | |
| | ORF Start: ATG at 221 | | ORF Stop: TGA at 2630 |
| | SEQ ID NO: 126 | 803 aa | MW at 90987.8 kD |

| | |
|---|---|
| NOV28b, | MAEAHQAVGFRPSLTSDGAEVELSAPVLQEIYLSGLRSWKRHLSRFWNDFLTGVFPASPLSWLFLFS |
| CG148102-02 | |
| Protein Sequence | AIQLAWFLQLDPSLGLMEKIKELLPDWGGQHHGLRGVLAAALFASCLWGALIFTLHVALRLLLSYHG |
| | WLLEPHGAMSSPTKTWLALVRIFSGRHPMLFSYQRSLPRQPVPSVQDTVRKYLESVRPILSDEDFDW |
| | TAVLAQEFLRLQASLLQWYLRLKSWWASNYVSDWWEEFVYLRSRNPLMVNSNYYMMDFLYVTPTPLQ |
| | AARAGNAVHALLLYRHRLNRQEIPPTLLMGMRPLCSAQYEKIFNTTRIPGVQKDYIRHLHDSQHVAV |
| | FHRGRFFRMGTHSRNSLLSPRALEQQFQRILDDPSPACPHEEHLAALTAAPRGTWAQVRTSLKTQAA |
| | EALEAVEGAAFFVSLDAEPAGLTREDPAASLDAYAHALLAGRGHDRWFDKSFTLIVFSNGKLGLSVE |
| | HSWADCPISGHMWEFTLATECFQLGYSTDGHCKGHPDPTLPQPQRLQWDLPDQIHSSISLALRGAKI |
| | LSENVDCHVVPFSLFGKSFIRRCHLSSDSFIQIALQLAHFRDRGQFCLTYESAMTRLFLEGRTETVR |
| | SCTREACNFVRAMEDKEKTDPQCLALFRVAVDKHQALLKAAMSGQGVDRHLFALYIVSRFLHLQSPF |
| | LTQVHSEQWQLSTSQIPVQQMHLFDVHNYPDYVSSGGGFGPADDHGYGVSYIFMGDGMITFHISSKK |
| | SSTKTDSHRLGQHIEDALLDVASLFQAGQHFKRRFRGSGKENSRHRCGFLSRQTGASKASMTSTDF |

Sequence comparison of the above protein sequences yields the following sequence relationships shown in Table 28B.

TABLE 28B
Comparison of NOV28a against NOV28b.

| | NOV28a Residues/ | Identities/Similarities |
|---|---|---|
| Protein Sequence | Match Residues | for the Matched Region |
| NOV28b | 1 . . . 751 | 717/806 (88%) |
| | 1 . . . 803 | 719/806 (88%) |

Further analysis of the NOV28a protein yielded the following properties shown in Table 28C.

TABLE 28C
Protein Sequence Properties NOV28a

| | |
|---|---|
| PSort analysis: | 0.7900 probability located in plasma membrane; |
| | 0.6400 probability located in microbody |
| | (peroxisome); 0.3000 probability |
| | located in Golgi body; 0.2000 probability |
| | located in endoplasmic reticulum (membrane) |
| SignalP analysis: | Cleavage site between residues 5 and 6 |

A search of the NOV28a protein against the Geneseq database, a proprietary database that contains sequences published in patents and patent publication, yielded several homologous proteins shown in Table 28D.

TABLE 28D
Geneseq Results for NOV28a

| | | NOV28a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAY79220 | Human transferase | 1 . . . 751 | 739/806 (91%) | 0.0 |
| | TRNSFS-12 - Homo sapiens, | 1 . . . 803 | 742/806 (91%) | |
| | 803 aa. [WO200014251-A2, | | | |
| | 16-MAR-2000] | | | |
| AA1E10322 | Human carnitine | 1 . . . 751 | 739/806 (91%) | 0.0 |
| | acyltransferase, 26886− | 1 . . . 803 | 742/806 (91%) | |
| | Homo sapiens, 803 aa. | | | |
| | [WO200166759-A2, | | | |
| | 13-SEP-2001] | | | |
| AAW14438 | Type I carnitine palmitoyl | 1 . . . 711 | 375/770 (48%) | 0.0 |
| | transferase-like protein - | 1 . . . 766 | 495/770 (63%) | |
| | Homo sapiens, 772 aa. | | | |
| | [JP09009969-A, | | | |
| | 14-JAN-1997] | | | |
| ABG04960 | Novel human diagnostic | 224 . . . 571 | 337/381 (88%) | 0.0 |
| | protein #4951 - Homo | 92 . . . 471 | 339/381 (88%) | |
| | sapiens, 521 aa. | | | |
| | [WO200175067-A2, | | | |
| | 11-OCT-2001] | | | |
| ABB67527 | Drosophila melanogaster | 1 . . . 717 | 315/775 (40%) | e−161 |
| | polypeptide SEQ ID NO | 1 . . . 765 | 447/775 (57%) | |
| | 29373 - Drosophila | | | |
| | melanogaster, 780 aa. | | | |
| | [WO200171042-A2, | | | |
| | 27-SEP-2001] | | | |

In a BLAST search of public sequence datbases, the NOV28a protein was found to have homology to the proteins shown in the BLASTP data in Table 28E.

TABLE 28E
Public BLASTP Results for NOV28a

| | | NOV28a | Identities/ | |
|---|---|---|---|---|
| | | Residuesl | Similarities for | |
| Protein Accession | | Match | the Matched | Expect |
| Number | Protein/Organism/Length | Residues | Portion | Value |
| Q8TCG5 | Carnitine | 1 . . . 751 | 740/806 (91%) | 0.0 |
| | palmitoyltransferase IC - | 1 . . . 803 | 742/806 (91%) | |
| | Homo sapiens (Human), 803 | | | |
| | aa. | | | |
| CAC88591 | Sequence 1 from Patent | 1 . . . 751 | 739/806 (91%) | 0.0 |
| | WO0166759 - Homo sapiens | 1 . . . 803 | 742/806 (91%) | |
| | (Human), 803 aa | | | |
| AAH29104 | Similar to carnitine | 1 . . . 751 | 729/806 (90%) | 0.0 |
| | palmitoyltransferase IC - | 1 . . . 792 | 731/806 (90%) | |
| | Homo sapiens (Human), 792 | | | |
| | aa. | | | |
| P32198 | Carnitine | 1 . . . 710 | 394/768 (51%) | 0.0 |
| | O-pahmitoyltransferase I, | 1 . . . 765 | 524/768 (67%) | |
| | mitochondrial liver isoform | | | |
| | (EC 2.3.1.21) (CPT I) | | | |
| | (CPTI-L) - Rattus norvegicus | | | |
| | (Rat), 773 aa. | | | |
| Q9BWK0 | Similar to carnitine | 1 . . . 690 | 381/748 (50%) | 0.0 |
| | palmitoyltransferase I, liver - | 1 . . . 745 | 510/748 (67%) | |
| | Homo sapiens (Human), 756 | | | |
| | aa. | | | |

PFam analysis predicts that the NOV28a protein contains the domains shown in the Table 28F.

TABLE 28F
Domain Analysis of NOV28a

| | NOV28a | Identities/ | |
|---|---|---|---|
| | Match | Similarities for | |
| Pfam Domain | Region | the Matched Region | Expect Value |
| Carn₁₃ acyltransf | 162 . . . 708 | 208/680 (31%) | 1.5e−167 |
| | | 437/680 (64%) | |

### Example 29

The NOV29 clone was analyzed, and the nucleotide and encoded polypeptide sequences are shown in Table 29A.

TABLE 29A
NOV29 Sequence Analysis

| | SEQ ID NO: 127 | 1776 bp | |
|---|---|---|---|
| NOV29a, | ACTAAAGCCTGCAGAGACCTCTGAAGGAAAACCTGTCCCGGGCTCTGTCACTTCACACCCATGGCTA | | |
| CG148431-01 | | | |
| DNA Sequence | ACCCTGGAGGTGGTGCTGTTTGCAACGGGAAACTTCACAATCACAAGAAACAGAGCAATGGCTCACA | | |
| | AAGCAGAAACTGCACAAAGAATGGAATAGTGAAGGAAGCCCAGCAAAATGGGAAGCCACATTTTTAT | | |
| | GATAAGCTCATTGTTGAATCGTTTGAGGAAGCACCCCTTCATGTTATGGTTTTCACTTACATGGGAT | | |
| | ATGGAATTGGAACCCTGTTTGGCTATCTCAGAGACTTTTTAAGAAACTGGGGAATAGAAAAATGCAA | | |
| | CGCAGCTGTGGAAAGAAAAGAACAAAAAGATTTTGTGCCACTGTATCAAGACTTTGAAAATTTTTAT | | |
| | ACAAGAAACCTTTACATGCGAATCAGAGACAACTGGAACCGGCCCATCTGCAGTGCCCCAGGGCCTC | | |
| | TGTTTGATTTGATGGAGAGGGTATCAGACGACTATAACTGGACGTTTAGGTTTACTGGAAGAGTCAT | | |
| | CAAAGATGTCATCAACATGGGCTCCTATAACTTCCTTGGTCTTGCAGCCAAGTATGATGAGTCTATG | | |
| | AGGACAATAAAGGATGTTTTAGAGGTGTATGGCACAGGCGTGGCCAGCACCAGGCATGAAATGGGCA | | |
| | CCTTGGATAAGCACAAGGAGTTGGAGGACCTTGTGGCTAAGTTCCTGAATGTGGAAGCAGCTATGGT | | |
| | CTTTGGGATGGGATTCGCAACTAACTCAATGAATATCCCAGCATTAGTTGGAAAGGGATGCCTCATT | | |
| | TTAAGTGATGAGTTAAACCACACATCGCTTGTGCTTGGGGCCCGACTCTCAGGTGCAACCATAAGAA | | |
| | TCTTCAAACACAACAACACACAAAGCCTAGAGAAGCTCCTGAGAGATGCTGTCATCTATGGCCAGCC | | |
| | TCGAACCCGCAGAGCTTGGAAAAAGATTCTCATCCTGGTGGAGGGTGTCTACAGCATGGAAGGTTCC | | |
| | ATCGTGCATCTGCCCCAGATCATAGCTCTAAAGAAGAAATACAAGGCTTACCTCTACATAGATGAAG | | |
| | CTCACAGTATTGGGGCCGTGGGCCCAACCGGCCGGGGTGTCACGGAGTTCTTTGGACTAGACCCTCA | | |
| | TGAAGTTGATGTGCTCATGGGCACATTCACCAAAAGTTTTGGAGCTTCAGGAGGTTACATAGCTGGA | | |
| | AGGAAGGACCTCGTGGATTATTTACGGGTTCACTCGCATAGTGCTGTTTATGCTTCATCCATGAGCC | | |
| | CACCGATAGCAGAGCAAATCATCAGATCACTAAAACTTATCATGGGACTGGATGGGACCACTCAAGG | | |
| | GCTGCAGAGAGTACAGCAACTTGCGAAAAACACAAGATACTTCAGACAAAGACTGCAGGAAATGGGA | | |
| | TTCATTATCTATGGCAATGAGAATGCTTCTGTTGTTCCTCTGCTTCTTTATATGCCTGGTAAAGTAG | | |
| | CGGCTTTTGCAAGGCATATGCTAGAGAAAAAAATTGGAGTGGTGGTCGTGGGATTTCCAGCCACTCC | | |
| | CCTCGCAGAAGCTCGGGCTCGGTTTTGTGTTTCAGCGGCACATACCCGGGAGATGTTAGACACGGTT | | |
| | TTAGAAGCTCTTGATGAAATGGGTGATCTCTTGCAACTGAAATATTCCCGGCACAAGAAGTCAGCAC | | |
| | GTCCTGAGCTCTATGATGAGACGAGCTTTGAACTCGAAGATTAAGTTTCCTGGTCCTGAATGACACA | | |
| | TAAAGACTTTGCGAGAAAGACCTCCCTCCTTGCC | | |
| | ORF Start: ATG at 61 | | ORF Stop: TAA at 1717 |
| | SEQ ID NO: 128 | 1552 aa | MW at 62048.9 kD |
| NOV29a, | MANPGGGAVCNGKLHNHKKQSNGSQSRNCTKNGIVKEAQQNGKPHFYDKLIVESFEEAPLHVMVFTY | | |
| CG148431-01 | | | |
| Protein Sequence | MGYGIGTLFGYLRDFLRNWGIEKCNAAVERKEQKDFVPLYQDFENFYTRNLYMRIRDNWNRPICSAP | | |
| | GPLFDLMERVSDDYNWTFRFTGRVIKDVINMGSYNFLGLAAKYDESMRTIKDVLEVYGTGVASTRHE | | |
| | MGTLDKHKELEDLVAKFLNVEAAMVFGMGFATNSMNIPALVGKGCLILSDELNHTSLVLGARLSGAT | | |
| | IRIFKHNNTQSLEKLLRDAVIYGQPRTRRAWKKILILVEGVYSMEGSIVHLPQIIALKKKYKAYLYI | | |
| | DEAHSIGAVGPTGRGVTEFFGLDPHEVDVLMGTFTKSFGASGGYIAGRKDLVDYLRVHSHSAVYASS | | |
| | MSPPIAEQIIRSLKLIMGLDGTTQGLQRVQQLAKNTRYFRQRLQEMGFIIYGNENASVVPLLLYMPG | | |
| | KVAAFARHMLEKKIGVVVVGFPATPLAEARARFCVSAAHTREMLDTVLEALDEMGDLLQLKYSRHKK | | |
| | SARPELYDETSFELED | | |
| | SEQ ID NO: 129 | 1492 bp | |
| NOV29b, | CACCGGATCCACCATGGCTAACCCTGGAGGTGGTGCTGTTTGCAACGGGAAACTTCACAATCACAAG | | |
| CG148431-02 | | | |
| DNA Sequence | AAACAGAGCAATGGCTCACAAAGCAGAAACTGCACAAAGAATGGAATAGTGAAGGAAGCCCAGGATT | | |
| | TTGTGCCACTGTATCAAGACTTTGAAAATTTTTATACAAGAAACCTTTACATGCGAATCAGAGACAA | | |
| | CTGGAACCGGCCCATCTGCAGTGCCCCAGGGCCTCTGTTTGATGTGATGGAGAGGGTATCGGACGAC | | |
| | TATAACTGGACGTTTAGGTTTACTGGAAGAGTCATCAAAGATGTCATCAACATGGGCTCCTATAACT | | |
| | TCCTTGGTCTTGCAGCCAAGTATGATGAGTCTATGAGGACAATAAAGGATGTTTTAGAGGTGTATGG | | |
| | CACAGGCGTGGCCAGCACCAGGCATGAAATGGGCACCTTGGATAAGCACAAGGAGTTGGAGGACCTT | | |
| | GTGGCTAAGTTCCTGAATGTGGAAGCAGCTATGGTCTTTGGGATGGGATTCGCAACTAACTCAATGA | | |
| | ATATCCCAGCATTAGTTGGAAAGGGATGCCTCATTTTAAGTGATGAGTTAAACCACACATCGCTTGT | | |
| | GCTTGGGGCCCGACTCTCAGGTGCAACCATAAGAATCTTCAAACACAACAACACACAAAGCCTAGAG | | |
| | AAGCTCCTGAGAGATGCTGTCATCTATGGCCAGCCTCGAACCCGCAGAGCTTGGAAAAAGATTCTCA | | |
| | TCCTGGTGGAGGGTGTCTACAGCATGGAAGGTTCCATCGTGCATCTGCCCCAGATCATAGCTCTAAA | | |
| | GAAGAAATACAAGGCTTACCTCTACATAGATGAAGCTCACAGTATTGGGGCCGTGGGCCCAACCGGC | | |
| | CGGGGTGTCACGGAGTTCTTTGGACTAGACCCTCATGAAGTTGATGTGCTCATGGGCACATTCACCA | | |
| | AAAGTTTTGGAGCTTCAGGAGGTTACATAGCTGGAAGGAAGGACCTCGTGGATTATTTACGGGTTCA | | |
| | CTCGCATAGTGCTGTTTATGCTTCATCCATGAGCCCACCGATAGCAGAGCAAATCATCAGATCACTA | | |
| | AAACTTATCATGGGACTGGATGGGACCACTCAAGGGCTGCAGAGAGTACAGCAACTTGCGAAAAACA | | |
| | CAAGATACTTCAGACAAAGACTGCAGGAAATGGGATTCATTATCTATGGCAATGAGAATGCTTCTGT | | |
| | TGTTCCTCTGCTTCTTTATATGCCTGGTAAAGTAGCGGCTTTTGCAAGGCATATGCTAGAGAAAAAA | | |
| | ATTGGAGTGGTGGTCGTGGGATTTCCAGCCACTCCCCTCGCAGAAGCTCGGGCTCGGTTTTGTGTTT | | |
| | CAGCGGCACATACCCGGGAGATGTTAGACACGGTTTTAGAAGCTCTTGATGAAATGGGTGATCTCTT | | |
| | GCAACTGAAATATTCCCGGCACAAGAAGTCAGCACGTCCTGAGCTCTATGATGAGACGAGCTTTGAA | | |
| | CTCGAAGATCTCGAGGGC | | |
| | ORF Start: ATG at 14 | | ORF Stop: at 1484 |
| | SEQ ID NO: 130 | 490 aa | MW at 54766.5 kD |

| | |
|---|---|
| NOV29b, | MANPGGGAVCNGKLHNHKKQSNGSQSRNCTKNGIVKEAQDFVPLYQDFENFYTRNLYMRIRDNWNRP |
| CG148431-02 | |
| Protein Sequence | ICSAPGPLFDVMERVSDDYNWTFRFTGRVIKDVINMGSYNFLGLAAKYDESMRTIKDVLEVYGTGVA |
| | STRHEMGTLDKHKELEDLVAKFLNVEAAMVFGMGFATNSMNIPALVGKGCLILSDELNHTSLVLGAR |
| | LSGATIRIFKHNNTQSLEKLLRDAVIYGQPRTRRAWKKILILVEGVYSMEGSIVHLPQIIALKKKYK |
| | AYLYIDEAHSIGAVGPTGRGVTEFFGLDPHEVDVLMGTFTKSFGASGGYIAGRKDLVDYLRVHSHSA |
| | VYASSMSPPIAEQIIRSLKLIMGLDGTTQGLQRVQQLAKNTRYFRQRLQEMGFIIYGNENASVVPLL |
| | LYMPGKVAAFARHMLEKKIGVVVVGFPATPLAEARARFCVSAAHTREMLDTVLEALDEMGDLLQLKY |
| | SRHKKSARPELYDETSFELED |

Sequence comparison of the above protein sequences yields the following sequence relationships shown in Table 29B.

TABLE 29B
Comparison of NOV29a against NOV29b.

| | NOV29a Residues/ | Identities/Similarities |
|---|---|---|
| Protein Sequence | Match Residues | for the Matched Region |
| NOV29b | 98 . . . 552 | 438/455 (96%) |
| | 36 . . . 490 | 440/455 (96%) |

Further analysis of the NOV29a protein yielded the following properties shown in Table 29C.

TABLE 29C
Protein Sequence Properties NOV29a

| | |
|---|---|
| PSort analysis: | 0.4761 probability located in microbody (peroxisome); |
| | 0.3000 probability located in nucleus; |
| | 0.2077 probability located in lysosome (lumen); |
| | 0.1000 probability located in mitochondrial matrix |
| | space |
| SignalP analysis: | No Known Signal Sequence Predicted |

A search of the NOV29a protein against the Geneseq database, a proprietary database that contains sequences published in patents and patent publication, yielded several homologous proteins shown in Table 29D.

TABLE 29D
Geneseq Results for NOV29a

| | | NOV29a | | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAE22153 | Human TRNFR-15 protein - | 1 . . . 552 | 551/552 (99%) | 0.0 |
| | Homo sapiens, 552 aa. | 1 . . . 552 | 552/552 (99%) | |
| | [WO200226950-A2, | | | |
| | 04-APR-2002] | | | |
| AAG73598 | Human colon cancer antigen | 201 . . . 549 | 269/352 (76%) | e−158 |
| | protein SEQ ID NO: 4362 - | 38 . . . 387 | 316/352 (89%) | |
| | Homo sapiens, 391 aa. | | | |
| | [WO200122920-A2, | | | |
| | 05-APR-2001] | | | |
| ABB60160 | Drosophila melanogaster | 54 . . . 543 | 256/491 (52%) | e−151 |
| | polypeptide SEQ ID NO | 114 . . . 597 | 350/491 (71%) | |
| | 7272 - Drosophila | | | |
| | melanogaster 597 aa. | | | |
| | [WO200171042-A2, | | | |
| | 27-SEP-2001] | | | |
| AAE21820 | Human serine | 47 . . . 276 | 228/230 (99%) | e−133 |
| | palmitoyltransferase | 1 . . . 230 | 230/230 (99%) | |
| | (SPT)-like enzyme #2 - | | | |
| | Homo sapiens, 230 aa. | | | |
| | [WO200224884-A2, | | | |
| | 28-MAR-2002] | | | |
| AAY32003 | Rice serine | 59 . . . 541 | 237/485 (48%) | e−133 |
| | palmitoyltransferase Lcb2 | 5 . . . 483 | 333/485 (67%) | |
| | subunit - Oryza sativa, 489 | | | |
| | aa. [WO9949021-A1, | | | |
| | 30-SEP-1999] | | | |

In a BLAST search of public sequence datbases, the NOV29a protein was found to have homology to the proteins shown in the BLASTP data in Table 29E.

TABLE 29E
Public BLASTP Results for NOV29a

| | | NOV29a | | |
|---|---|---|---|---|
| Protein | | Residues/ | Identities/ | |
| Accession | | Match | Similarities for the | Expect |
| Number | Protein/Organism/Length | Residues | Matched Portion | Value |
| Q9UGB6 | DJ718P11.1.1 (Novel class II | 102 . . . 515 | 414/414 (100%) | 0.0 |
| | aminotransferase similar to | 1 . . . 414 | 414/414 (100%) | |
| | serine palmotyltransferase | | | |
| | (Isoform 1)) - Homo sapiens | | | |
| | (Human), 414 aa (fragment). | | | |
| O15270 | Serine palmitoyltransferase 2 | 7 . . . 549 | 383/546 (70%) | 0.0 |
| | (EC 2.3.1.50) (Long chain | 18 . . . 558 | 449/546 (82%) | |
| | base biosynthesis protein 2) | | | |
| | (LCB 2) | | | |
| | (Serine-palmitoyl-CoA | | | |
| | transferase 2) (SPT 2) - | | | |
| | Homo sapiens (Human), 562 | | | |
| | aa. | | | |
| P97363 | Serine palmitoyltransferase 2 | 7 . . . 549 | 379/546 (69%) | 0.0 |
| | (EC 2.3.1.50) (Long chain | 18 . . . 556 | 449/546 (81%) | |
| | base biosynthesis protein 2) | | | |
| | (LCB 2) | | | |
| | (Serine-palmitoyl-CoA | | | |
| | transferase 2) (SPT 2) - Mus | | | |
| | musculus (Mouse), 560 aa. | | | |
| JC5180 | serine C-palmitoyltransferase | 7 . . . 549 | 378/546 (69%) | 0.0 |
| | (EC 2.3.1.50) Lcb2 chain - | 18 . . . 556 | 449/546 (82%) | |
| | mouse, 560 aa. | | | |
| O54694 | Serine palmitoyltransferase 2 | 7 . . . 549 | 377/546 (69%) | 0.0 |
| | (EC 2.3.1.50) (Long chain | 18 . . . 556 | 446/546 (81%) | |
| | base biosynthesis protein 2) | | | |
| | (LCB 2) | | | |
| | (Serine-palmitoyl-CoA | | | |
| | transferase 2) (SPT 2) - | | | |
| | Cricetulus griseus (Chinese | | | |
| | hamster), 560 aa. | | | |

PFam analysis predicts that the NOV29a protein contains the domains shown in the Table 29F.

TABLE 29F
Domain Analysis of NOV29a

| | | Identities/ | |
|---|---|---|---|
| | | Similarities | |
| | NOV29a | for the Matched | |
| Pfam Domain | Match Region | Region | Expect Value |
| aminotran_1_2 | 193 . . . 521 | 71/363 (20%) | 2.6e−29 |
| | | 237/363 (65%) | |

### Example 30

The NOV30 clone was analyzed, and the nucleotide and encoded polypeptide sequences are shown in Table 30A.

TABLE 30A
NOV30 Sequence Analysis

| | SEQ ID NO: 131 | 576 bp | |
|---|---|---|---|
| NOV30a, | TGAGCCAGCCCCGGATGACCCTGCGACCTGGAACAATGCGGCTGGCCTGCATGTTCTCTTCCATCCT | | |
| CG148888-01 | | | |
| DNA Sequence | GCTGTTCGGAGCTGCAGGCCTCCTCCTCTTCATCAGCCTGCAGGACCCTACGGAGCTCGCCCCCCAG | | |
| | CAGGTGCCAGGAATAAAGTTCAACATCAGGCCAAGGCAGCCCCACCACGACCTCCCACCAGGCGGCT | | |
| | CTGGGGTGCGTTTTCCCGAGTTCGTCCAGTACCTGCTGGACGTGCACCGGCCCGTGGGGATGGACAT | | |
| | TCACTGGGACCATGTCAGCCGGCTCTGCAGCCCCTGCCTCATCGACTACGATTTCGTAGGCAAGTTC | | |
| | GAGAGCATGGAGGACGATGCCAACTTCTTCCTGAGCCTCATCCGCGCGCCGCGGAACCTGACCTTCC | | |
| | CCCGGTTCAAGGACCGGCACTCGCAGGAGGCGCGGACCACAGCGAGGATCGCCCACCAGTACTTCGC | | |
| | CCAACTCTCGGCCCTGCAAAGGCAGCGCACCTACGACTTCTACTACATGGATTACCTGATGTTCAAC | | |
| | TATTCCAAGCCCTTTACAGATCTGTACTGAGGGGCGCCGC | | |
| | ORF Start: ATG at 15 | | ORF Stop: TGA at 564 |
| | SEQ ID NO: 132 | 183 aa | MW at 21347.3 kD |

| | |
|---|---|
| NOV30a, | MTLRPGTMRLACMFSSILLFGAAGLLLFISLQDPTELAPQQVPGIKFNIRPRQPHHDLPPGGSGVRF |
| CG148888-01 | |
| Protein Sequence | PEFVQYLLDVHRPVGMDIHWDHVSRLCSPCLIDYDFVGKFESMEDDANFFLSLIRAPRNLTFPRFKD |
| | RHSQEARTTARIAHQYFAQLSALQRQRTYDFYYMDYLMFNYSKPFTDLY |

Further analysis of the NOV30a protein yielded the following properties shown in Table 30B.

TABLE 30B
Protein Sequence Properties NOV30a

| | |
|---|---|
| PSort analysis: | 0.8650 probability located in lysosome (lumen); |
| | 0.8200 probability located in outside; |
| | 0.3657 probability located in microbody (peroxisome); |
| | 0.1000 probability located in endoplasmic reticulum |
| | (membrane) |
| SignalP analysis: | Cleavage site between residues 38 and 39 |

A search of the NOV30a protein against the Geneseq database, a proprietary database that contains sequences published in patents and patent publication, yielded several homologous proteins shown in Table 30C.

TABLE 30C
Geneseq Results for NOV30a

| | | NOV30a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB53266 | Human polypeptide #6 - | 62 . . . 183 | 121/122 (99%) | 4e−69 |
| | Homo sapiens, 424 aa. | 303 . . . 424 | 121/122 (99%) | |
| | [WO200181363-AI, | | | |
| | 01-NOV-2001] | | | |
| ABB53265 | Human polypeptide #5 - | 62 . . . 183 | 121/122 (99%) | 4e−69 |
| | Homo sapiens, 628 aa. | 507 . . . 628 | 121/122 (99%) | |
| | [WO200181363-A1, | | | |
| | 01-NOV-2001] | | | |
| AAE15437 | Human drug metabolising enzyme | 62 . . . 183 | 121/122 (99%) | 4e−69 |
| | (DME)-4 - Homo sapiens, 396 aa. | 275 . . . 396 | 121/122 (99%) | |
| | [WO200179468-A2, | | | |
| | 25-OCT-2001] | | | |
| AAB85083 | Human interleukin-6 (IL-6) | 62 . . . 183 | 121/122 (99%) | 4e−69 |
| | like polypeptide - Homo | 50 . . . 171 | 121/122 (99%) | |
| | sapiens, 171 aa. | | | |
| | [WO200142484-A1, | | | |
| | 14-JUN-2001] | | | |
| AAM24429 | Murine EST encoded protein | 62 . . . 183 | 121/122 (99%) | 4e−69 |
| | SEQ ID NO: 1954 - Mus | 303 . . . 424 | 121/122 (99%) | |
| | musculus, 424 aa. | | | |
| | [WO200154477-A2, | | | |
| | 02-AUG-2001] | | | |

In a BLAST search of public sequence datbases, the NOV30a protein was found to have homology to the proteins shown in the BLASTP data in Table 30D.

TABLE 30D
Public BLASTP Results for NOV30a

| | | NOV30a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q9H3N2 | GalNAc 4-sulfotransferase | 62 . . . 183 | 121/122 (99%) | 1e−68 |
| | (GalNAc-4-O-sulfotransferase 1) | 303 . . . 424 | 121/122 (99%) | |
| | (Carbohydrate (N-acetylgalactosamine | | | |
| | 4-0) sulfotransferase 8) (Hypothetical | | | |
| | 48.8 kDa protein) - Homo sapiens | | | |
| | (Human), 424 aa. | | | |
| Q9H2A9 | N-acetylgalactosamine-4-O- | 62 . . . 183 | 120/122 (98%) | 4e−68 |
| | sulfotransferase - Homo sapiens | 303 . . . 424 | 120/122 (98%) | |
| | (Human), 424 aa. | | | |
| Q9BXH4 | GalNAc-4-sulfotransferase 2 - Homo | 62 . . . 179 | 77/118 (65%) | 1e−44 |
| | sapiens (Human), 443 aa. | 325 . . . 442 | 95/118 (80%) | |
| Q9BXH3 | GalNAc-4-sulfotransferase 2 - Homo | 62 . . . 179 | 77/118 (65%) | 1e−44 |
| | sapiens (Human), 358 aa. | 240 . . . 357 | 95/118 (80%) | |
| Q9BZW9 | N-acetylgalactosamine | 62 . . . 179 | 77/118 (65%) | 1e−44 |
| | 4-O-sulfotransferase 2 GalNAc4ST-2 - | 320 . . . 437 | 95/118 (80%) | |
| | Homo sapiens (Human), 438 aa. | | | |

### Example 31

The NOV31 clone was analyzed, and the nucleotide and encoded polypeptide sequences are shown in Table 31A.

TABLE 31A
NOV31 Sequence Analysis

| | SEQ ID NO: 133 | 2325 bp | |
|---|---|---|---|
| NOV31a, | CCCAGGCCGGACAAGCGTCCCGAAAGCCCCGGGAGAGACTAAGAAGCAATCCTCCCACGCGCTTTCT | | |
| CG149008-01 | | | |
| DNA Sequence | CCCACCCTCGGGCCACTGAGACCGAGGGACAGAGGGCCGCCCTCGCCCCGCCGAGGCCCCGCCTCCC | | |
| | GCTCGCCCGCCCGCGCCTCCAGCGGAAGCCGGAAGCAAAAGCGGGTCCTGCTAGCCCCGCGGCTCCG | | |
| | AACTCGGTGGTCCTGGAAGCTCCGCAGGATGGGGGAGAAGATGGCGGAAGACGAGAGGTTCCCCAAT | | |
| | ACAACTCATGAGGGTTTCAATGTCACCCTCCACACCACCCTGGTTGTCACGACGAAACTGGTGCTCC | | |
| | CGACCCCTGGCAAGCCCATCCTCCCCGTGCAGACAGGGGAGCAGGCCCAGCAAGAGGAGCAGTCCAG | | |
| | CGGCATGACCATTTTCTTCACCCTCCTTGTCCTAGCTATCTGCATCATATTGGTGCATTTACTGATC | | |
| | CGATACAGATTACATTTCTTGCCAGAGAGTGTTGCTGTTGTTTCTTTAGGTATTCTCATGGGAGCAG | | |
| | TTATAAAAATTATAGAGTTTAAAAAACTGGCGAATTGGAAGGAAGAAGAAATGTTTCGTCCAAACAT | | |
| | GTTTTTCCTCCTCCTGCTTCCCCCTATTATCTTTGAGTCTGGATATTCATTACACAAGGTGAGACTC | | |
| | AGGCACACATTGGGTAACTTCTTTCAAAATATTGGTTCCATCACCCTGTTTGCTGTTTTTGGGACGG | | |
| | CAATCTCCGCTTTTGTAGTAGGTGGAGGAATTTATTTTCTGGGTCAGGCTGATGTAATCTCTAAACT | | |
| | CAACATGACACACAGTTTTGCGTTTGGCTCCCTAATATCTGCTGTCGATCCAGTGGCCACTATTGCC | | |
| | ATTTTCAATGCACTTCATGTGGACCCCGTGCTCAACATGCTGGTCTTTGCAGAAAGTATTCTCAACG | | |
| | ATGCAGTCTCCATTGTTCTGACCAACACAGCTGAAGGTTTAACAAGAAAAAATATGTCAGATGTCAG | | |
| | TGGGTGGCAAACATTTTTACAAGCCCTTGACTACTTCCTCAAAATGTTCTTTGGCTCTGCAGCGCTC | | |
| | GGCACTCTCACTGGCTTAATTTCTGCATTAGTGCTGAAGCATATTGACTTGAGGAAAACGCCTTCCT | | |
| | TGGAGTTTGGCATGATGATCATTTTTGCTTATCTGCCTTATGGGCTTGCAGAAGGAATCTCACTCTC | | |
| | AGGCATCATGGCCATCCTGTTCTCAGGCATCGTGATGTCCCACTACACGCACCATAACCTCTCCCCA | | |
| | GTCACCCAGATCCTCATGCAGCAGACCCTCCGCACCGTGGCCTTCTTATGTGAAACATGTGTGTTTG | | |
| | CATTTCTTGGCCTGTCCATTTTTAGTTTTCCTCACAAGTTTGAAATTTCCTTTGTCATCTGGTGCAT | | |
| | AGTGCTTGTACTATTTGGCAGAGCGGTAAACATTTTCCCTCTTTCCTACCTCCTGAATTTCTTCCGG | | |
| | GATCATAAAATCACACCGAAGATGATGTTCATCATGTGGTTTAGTGGCCTGCGGGGAGCCATCCCCT | | |
| | ATGCCCTGAGCCTACACCTGGACCTGGAGCCCATGGAGAAGCGGCAGCTCATCGGCACCACCACCAT | | |
| | CGTCATCGTGCTCTTCACCATCCTGCTGCTGGGCGGCAGCACCATGCCCCTCATTCGCCTCATGGAC | | |
| | ATCGAGGACGCCAAGGCACACCGCAGGAACAAGAAGGACGTCAACCTCAGCAAGACTGAGAAGATGG | | |
| | GCAACACTGTGGAGTCGGAGCACCTGTCGGAGCTCACGGAGGAGGAGTACGAGGCCCACTACATCAG | | |
| | GCGGCAGGACCTTAAGGGCTTCGTGTGGCTGGACGCCAAGTACCTGAACCCCTTCTTCACTCGGAGG | | |
| | CTGACGCAGGAGGACCTGCACCACGGGCGCATCCAGATGAAAACTCTCACCAACAAGTGGTACGAGG | | |
| | AGGTACGCCAGGGCCCCTCCCGCTCCGAGGACGACGAGCAGGAGCTCCTCTGACGCCAGGTGCCAAG | | |
| | GCTTCAGGCAGGCAGGCCCAGGATGGGCGTTTGCTGCGCACAGACACTCAGCAGGGGCCTCGCAGAC | | |
| | ATGCCTGCATCCAGCAGCCCCTTCAAGACATAAGAGGGCGGGGCGAGGTACTGGCTGCACAGTCGCC | | |
| | TTAGTCCAGAACCTGACACGCCTCTGGAGCCAGGCGACTTCTTGGGAAACTGTCATCTCCCGACTCC | | |
| | TCCCTGAGCCAGCCTCCGCTCAGTGTGGCTCCTCAGCCCACAGAGGGGAGGGAGCATGGGGCCAGGT | | |
| | GCCAGTCATCTGTGAAGCTAGGGCGCCTACCCCCCCACCCGGAGGAC | | |
| | ORF Start: ATG at 230 | | ORF Stop: TGA at 1994 |
| | SEQ ID NO: 134 | 588 aa | MW at 66297.1 kD |

| | |
|---|---|
| NOV31a, | MGEKMAEEERFPNTTHEGFNVTLHTTLVVTTKLVLPTPGKPILPVQTGEQAQQEEQSSGMTIFFSLL |
| CG149008-01 | |
| Protein Sequence | VLAICIILVHLLIRYRLHFLPESVAVVSLCILMGAVIKIIEFKKLANWKEEEMFRPNMFFLLLLPPT |
| | IFESGYSLHKVRLRHTLCNFFQNIGSTTLFAVFGTAISAFVVGGGIYFLGQADVTSKLNMTDSFAFG |
| | SLISAVDPVATIAIFNALHVDPVLNMLVFGESILNDAVSIVLTNTAEGLTRKNMSDVSGWQTFLQAL |
| | DYFLKMFFGSAALGTLTGLISALVLKHIDLRKTPSLEFGMMIIFAYLPYGLAEGISLSGIMAILFSG |
| | IVMSHYTHHNLSPVTQILMQQTLRTVAFLCETCVFAFLGLSIFSFPHRFETSFVIWCIVLVLFGRAV |
| | NIFPLSYLLNFFRDHKITPKMMFIMWFSGLRGAIPYALSLHLDLEPMEKRQLIGTTTIVIVLFTILL |
| | LGGSTMPLIRLMDIEDAXAHRRNKKDVNLSKTEKMGNTVESEHLSELTEEEYEAHYIRRQDLKGFVW |
| | LDAKYLNPFFTRRLTQEDLHMGRIQMKTLTNKWYEEVRQGPSGSEDDEQELL |

Further analysis of the NOV31a protein yielded the following properties shown in Table 31B.

TABLE 31B
Protein Sequence Properties NOV31a

| | |
|---|---|
| PSort analysis: | 0.8000 probability located in plasma membrane; |
| | 0.4000 probability located in Golgi body; |
| | 0.3000 probability located in endoplasmic |
| | reticulum (membrane); 0.3000 probability located in |
| | microbody (peroxisome) |
| SignalP analysis: | Cleavage site between residues 40 and 41 |

A search of the NOV31a protein against the Geneseq database, a proprietary database that contains sequences published in patents and patent publication, yielded several homologous proteins shown in Table 31C.

TABLE 31C
Geneseq Results for NOV31a

| | | NOV31a | | |
|---|---|---|---|---|
| | | Residues/ | Identities/ | |
| Geneseq | Protein/Organism/Length | Match | Similarities for the | Expect |
| Identifier | [Patent #, Date] | Residues | Matched Region | Value |
| ABG61535 | Human transporter and ion | 1 . . . 588 | 581/588 (98%) | 0.0 |
| | channel, TRICH5, Incyte ID | 91 . . . 671 | 581/588 (98%) | |
| | 7476938CD1 - Homo | | | |
| | sapiens, 671 aa. | | | |
| | [WO200240541-A2, | | | |
| | 23-MAY-2002] | | | |
| AAM24062 | Human EST encoded protein | 274 . . . 588 | 315/315 (100%) | 0.0 |
| | SEQ ID NO: 1587 - Homo | 1 . . . 315 | 315/315 (100%) | |
| | sapiens, 315 aa. | | | |
| | [WO200154477-A2, | | | |
| | 02-AUG-2001] | | | |
| AAB29621 | Cat flea HMT Na/H | 8 . . . 584 | 329/585 (56%) | e−175 |
| | transporter, SEQ ID | 33 . . . 602 | 416/585 (70%) | |
| | NO: 1868 - Ctenocephalides | | | |
| | felis, 608 aa. | | | |
| | [WO200061621-A2, | | | |
| | 19-OCT-2000] | | | |
| ABB59364 | Drosophila melanogaster | 44 . . . 587 | 310/562 (55%) | e−170 |
| | polypeptide SEQ ID NO | 86 . . . 635 | 399/562 (70%) | |
| | 4884 - Drosophila | | | |
| | melanogaster, 649 aa. | | | |
| | [WO200171042-A2, | | | |
| | 27-SEP-2001] | | | |
| AA014196 | Human transporter and ion | 117 . . . 547 | 166/439 (37%) | 2e−72 |
| | channel TRICH-13 - Homo | 125 . . . 542 | 253/439 (56%) | |
| | sapiens, 631 aa. | | | |
| | [WO200204520-A2, | | | |
| | 17-JAN-2002] | | | |

In a BLAST search of public sequence datbases, the NOV31 a protein was found to have homology to the proteins shown in the BLASTP data in Table 31D.

TABLE 31D
Public BLASTP Results for NOV31a

| | | NOV31a | | |
|---|---|---|---|---|
| Protein | | Residues/ | Identities/ | |
| Accession | | Match | Similarities for the | Expect |
| Number | Protein/Organism/Length | Residues | Matched Portion | Value |
| BAA76783 | KIAA0939 protein - Homo | 1 . . . 588 | 581/588 (98%) | 0.0 |
| | sapiens (Human), 595 aa | 15 . . . 595 | 581/588 (98%) | |
| | (fragment). | | | |
| Q8R4D1 | Na—H exchanger isoform | 5 . . . 587 | 556/583 (95%) | 0.0 |
| | NHE8 - Mus musculus | 1 . . . 575 | 565/583 (96%) | |
| | (Mouse), 576 aa. | | | |
| Q9Y507 | DJ963K23.4 (Continues in | 152 . . . 588 | 437/437 (100%) | 0.0 |
| | dJ1041C10 (AL162615)) - | 1 . . . 437 | 437/437 (100%) | |
| | Homo sapiens (Human), 437 | | | |
| | aa (fragment). | | | |
| Q9Y2E8 | KIAA0939 protein - Homo | 182 . . . 588 | 405/407 (99%) | 0.0 |
| | sapiens (Human), 411 aa | 5 . . . 411 | 406/407 (99%) | |
| | (fragment). | | | |
| AAH34508 | Hypothetical protein - Mus | 209 . . . 587 | 366/379 (96%) | 0.0 |
| | musculus (Mouse), 388 aa | 9 . . . 387 | 374/379 (98%) | |
| | (fragment). | | | |

PFam analysis predicts that the NOV31a protein contains the domains shown in the Table 31E.

TABLE 31E
Domain Analysis of NOV31a

| | | Identities/ | |
|---|---|---|---|
| | NOV31a | Similarities | |
| | Match | for the Matched | Expect |
| Pfam Domain | Region | Region | Value |
| Na_H_Exchanger | 62 . . . 485 | 141/465 (30%) | 3.1e−98 |
| | | 345/465 (74%) | |

### Example 32

The NOV32 clone was analyzed, and the nucleotide and encoded polypeptide sequences are shown in Table 32A.

TABLE 32A
NOV32 Sequence Analysis

| | SEQ ID NO: 135 | 367 bp | |
|---|---|---|---|
| NOV32a, | ATGGCGGGGAGAAGGAAGCTCATCGCAGTGATCAGAGACAAGGACACGGTGACTGGTTTCCTGCTGG | | |
| CG149350-01 | | | |
| DNA Sequence | GCAGCATAGGGGAGCTTAACAAGAACTGCCACCCCAATTTCCTGGTGGTGGAGAAGGATACGACCAT | | |
| | CAATGAGATCGAAGACACTTTCCGGCAATTTCTAAACCGGGATGACACTGGCATCATCCTCATCAAC | | |
| | CAGTACATCGCAGAGATGGTGCAGCATGCCCTGGACACCCACCAGCACTCTATCCCTACTGTCCTGG | | |
| | AGATCCCCTCCAAGGAGCACCCATATGAGGACGCCAAGGACTCCACCCTGCGGAGGGCCAGGGGCAT | | |
| | GTTCACTGCCGAAGACCTGTGCTAGGGTCTTT | | |
| | ORF Start: ATG at 1 | | ORF Stop: TAG at 358 |
| | SEQ ID NO: 136 | 119 aa | MW at 13566.3 kD |
| NOV32a, | MAGRRKLIAVIRDKDTVTGFLLGSIGELNXNCHFNFLVVEKDTTINEIEDTFRQFLNRDDTGIILIN | | |
| CG149350-01 | | | |
| Protein Sequence | QYIAEMVQHALDTHQHSIPTVLEIPSKEHPYEDAKDSTLRRARGMFTAEDLC | | |
| | SEQ ID NO: 137 | 367 bp | |
| NOV32b, | ATGGCGGGGAGAAGGAAGCTCATCGCAGTGATCAGAGACAAGGACACGGTGACTCGTTTCCTGCTGG | | |
| CG149350-02 | | | |
| DNA Sequence | GCAGCATAGGGGAGCTTAACAAGAACTGCCACCCCAATTTCCTGGTGGTGGAGAAGGATACGACCAT | | |
| | CAATGAGATCGAAGACACTTTCCGGCAATTTCTAAACCGGGATGACACTGGCATCATCCTCATCAAC | | |
| | CAGTACATCGCAGAGATGGTGCAGCATGCCCTGGACACCCACCAGCACTCTATCCCTACTGTCCTGG | | |
| | AGATCCCCTCCAAGGAGCACCCATATGAGGACGCCAAGGACTCCACCCTGCGGAGGGCCAGGGGCAT | | |
| | GTTCACTGCCGAAGACCTGTGCTAGGGTCTTT | | |
| | ORF Start: ATG at 1 | | ORF Stop: TAG at 358 |
| | SEQ ID NO: 138 | 119 aa | MW at 13566.3 kD |

| | |
|---|---|
| NOV32b, | MAGRRKLIAVIRDKDTVTGFLLGSIGELNKNCHPNFLVVEKDTTINEIEDTFRQFLNRDDTGIILIN |
| CG149350-02 | |
| Protein Sequence | QYIAEMVQHALDTHQHSIPTVLEIPSKEHPYEDAKDSTLRRARGMFTAEDLC |

Sequence comparison of the above protein sequences yields the following sequence relationships shown in Table 32B.

TABLE 32B
Comparison of NOV32a against NOV32b.

| | | Identities/ |
|---|---|---|
| | NOV32a Residues/ | Similarities for |
| Protein Sequence | Match Residues | the Matched Region |
| NOV32b | 1 . . . 119 | 119/119 (100%) |
| | 1 . . . 119 | 119/119 (100%) |

Further analysis of the NOV32a protein yielded the following properties shown in Table 32C.

TABLE 32C
Protein Sequence Properties NOV32a

| | |
|---|---|
| PSort analysis: | 0.4852 probability located in mitochondrial matrix |
| | space; 0.4500 probability located in cytoplasm; |
| | 0.1957 probability located in mitochondrial inner |
| | membrane; 0.1957 probability located in |
| | mitochondrial intermembrane space |
| SignalP analysis: | No Known Signal Sequence Predicted |

A search of the NOV32a protein against the Geneseq database, a proprietary database that contains sequences published in patents and patent publication, yielded several homologous proteins shown in Table 32D.

TABLE 32D
Geneseq Results for NOV32a

| | | NOV32a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAW27337 | Human vacuolar ATPase 14 | 1 . . . 118 | 105/118 (88%) | 2e−54 |
| | kDa subunit hV-14B - Homo | 1 . . . 118 | 108/118 (90%) | |
| | sapiens, 119 aa. | | | |
| | [JP09168390-A, | | | |
| | 30-JUN-1997] | | | |
| AAW27336 | Human vacuolar ATPase 14 | 1 . . . 118 | 104/118 (88%) | 8e−54 |
| | kDa subunit hV-14A - Homo | 1 . . . 118 | 107/118 (90%) | |
| | sapiens, 119 aa. | | | |
| | [JP09168390-A, | | | |
| | 30-JUN-1997] | | | |
| ABB62928 | Drosophila melanogaster | 6 . . . 118 | 71/113 (62%) | 2e−38 |
| | polypeptide SEQ ID NO | 10 . . . 122 | 91/113 (79%) | |
| | 15576 - Drosophila | | | |
| | melanogaster, 124 aa. | | | |
| | [WO200171042-A2, | | | |
| | 27-SEP-2001] | | | |
| ABB57798 | Drosophila melanogaster | 6 . . . 114 | 58/109 (53%) | 7e−29 |
| | polypeptide SEQ ID NO 186 - | 10 . . . 118 | 84/109 (76%) | |
| | Drosophila melanogaster, | | | |
| | 124 aa. [WO200171042-A2, | | | |
| | 27-SEP-2001] | | | |
| AAG35989 | Zea mays protein fragment | 1 . . . 118 | 56/125 (44%) | 1e−27 |
| | SEQ ID NO: 44042 - Zea | 1 . . . 125 | 85/125 (67%) | |
| | mays subsp. mays, 130 aa. | | | |
| | [EP1033405-A2, | | | |
| | 06-SEP-2000] | | | |

In a BLAST search of public sequence datbases, the NOV32a protein was found to have homology to the proteins shown in the BLASTP data in Table 32E.

TABLE 32E
Public BLASTP Results for NOV32a

| | | NOV32a | Identities/ | |
|---|---|---|---|---|
| Protein | | Residues/ | Similarities for | |
| Accession | | Match | the Matched | |
| Number | Protein/Organism/Length | Residues | Portion | Expect Value |
| P50408 | Vacuolar ATP synthase | 1 . . . 118 | 104/118 (88%) | 1e−53 |
| | sunit F (EC 3.6.3.14) | 1 . . . 118 | 108/118 (91%) | |
| | (V-ATPase F subunit) | | | |
| | (Vacuolar proton pump F | | | |
| | subunit) (V-ATPase 14 kDa | | | |
| | subunit) - Rattus norvegicus | | | |
| | (Rat), 119 aa. | | | |
| Q16864 | Vacuolar ATP synthase | 1 . . . 118 | 104/118 (88%) | 2e−53 |
| | subunit F(EC 3.6.3.14) | 1 . . . 118 | 107/118 (90%) | |
| | (V-ATPase F subunit) | | | |
| | (Vacuolar proton pump F | | | |
| | subunit)(V-ATPase 14 kDa | | | |
| | subunit) - Homo sapiens | | | |
| | (Human), 119 aa. | | | |
| Q9D1K2 | 1110004G16Rik protein- | 1 . . . 118 | 103/118 (87%) | 5e−53 |
| | Mus musculus (Mouse), 119 | 1 . . . 118 | 108/118 (91%) | |
| | aa. | | | |
| Q28029 | Vacuolar ATP synthase | 10 . . . 118 | 97/109 (88%) | 7e−50 |
| | subunit F (EC 3.6.3.14) | 1 . . . 109 | 100/109 (90%) | |
| | (V-ATPase F subunit) | | | |
| | (Vacuolar proton pump F | | | |
| | subunit) (V-ATPase 14 kDa | | | |
| | subunit) - Bos taurus | | | |
| | (Bovine), 110 aa (fragment). | | | |
| Q9I8H3 | Vacuolar ATP synthase | 10 . . . 118 | 83/109 (76%) | 7e−43 |
| | subunit F (EC 3.6.3.14) | 1 . . . 109 | 94/109 (86%) | |
| | (V-ATPase F subunit) | | | |
| | (Vacuolar proton pump F | | | |
| | subunit) (V-ATPase 14 kDa | | | |
| | subunit) - Xenopus laevis | | | |
| | (African clawed frog), 110 aa | | | |
| | (fragment). | | | |

PFam analysis predicts that the NOV32a protein contains the domains shown in the Table 32F.

TABLE 32F
Domain Analysis of NOV32a

| | | Identities/ | |
|---|---|---|---|
| | | Similarities | |
| | | for the Matched | Expect |
| Pfam Domain | NOV32a Match Region | Region | Value |
| ATP-synt_F | 8 . . . 108 | 51/107 (48%) | 9.2e−43 |
| | | 90/107 (84%) | |

### Example 33

The NOV33 clone was analyzed, and the nucleotide and encoded polypeptide sequences are shown in Table 33A.

TABLE 33A
NOV33 Sequence Analysis

| | SEQ ID NO: 139 | 1510 bp | |
|---|---|---|---|
| NOV33a, | ATGGGTTCAGACTTTATGCCCTGAAAAGATCCTTCCAGCCCTGGCCATCTTGGACTTCTGGAGCTAC | | |
| CG149463-01 | | | |
| DNA Sequence | CCTGGCTCACAGGGGTCTTGTTGCCCTGGGTGTCCCCAGTTCTTGAAAAGAATCACCCTGGGAGGGC | | |
| | CCACACCCTGACCATCCCCCTTTATCCCTTCTGAGATGTTTGTTAGGAAGTCTGGGTCCAGGCGATA | | |
| | TCATTTCTTGTTCCATCCATGCAGGGGTTGCTTACCTCGGGTAGGAAACCCTCAGGCGGTGGCAGGT | | |
| | GCACAGGTAGGGGAGGATGGAGAGGGCAGTCGTGCCTGAAGCCCTGGATGGGCGGAGCTGACCCCCC | | |
| | AACACCAACTCTATCATGCCTGCTCCTCCCTGTCCCCCCAGAGCTGCCTGATCATTGCTACAGAATG | | |
| | AACTCTAGCCCAGCTGGGACCCCAAGTCCACAGCCCTCCAGGGCCAATGGGAACATCAACCTGGGGC | | |
| | CTTCAGCCAACCCAAATGCCCAGCCCACGGACTTCGACTTCCTCAAAGTCATCGGCAAAGGGAACTA | | |
| | CGGGAAGGTCCTACTGGCCAAGCGCAAGTCTGATGGGGCGTTCTATGCAGTGAAGGTACTACAGAAA | | |
| | AAGTCCATCTTAAACAAGAAAGAGCAGAGCCACATCATGGCAGAGCGCAGTGTGCTTCTGAAGAACG | | |
| | TGCGGCACCCCTTCCTCGTGGGCCTGCGCTACTCCTTCCAGACACCTGAGAAGCTCTACTTCGTGCT | | |
| | CGACTATGTCAACGGGGGAGAGCTCTTCTTCCACCTGCAGCGGGAGCGCCGGTTCCTGGAGCCCCGG | | |
| | GCCAGGTTCTACGCTGCTGAGGTGGCCAGCGCCATTGGCTACCTGCACTCCCTCAACATCATTTACA | | |
| | GGGATCTGAAACCAGAGAACATTCTCTTGGACTGCCAGTACTTGGCACCTGAAGTGCTTCGGAAAGA | | |
| | GCCTTATGATCGAGCAGTGGACTGGTGGTGCTTGGGGGCAGTCCTCTACGAGATGCTCCATGGCCTG | | |
| | CCGCCCTTCTACAGCCAAGATGTATCCCAGATGTATGAGAACATTCTGCACCAGCCGCTACAGATCC | | |
| | CCGGAGGCCGGACAGTGGCCGCCTGTGACCTCCTGCAAAGCCTTCTCCACAAGGACCAGAGGCAGCG | | |
| | GCTGGGCTCCAAAGCAGACTTTCTTGAGATTAAGAACCATGTATTCTTCAGCCCCATAAACTGGGAT | | |
| | GACCTGTACCACAAGAGCCTAACTCCACCCTTCAACCCAAATGTGACAGGACCTGCTGACTTGAAGC | | |
| | ATTTTGACCCAGAGTTCACCCAGGAAGCTGTGTCCAAGTCCATTGGCTGTACCCCCGACACTGTGGC | | |
| | CAGCAGCTCTGCCGCCTCAAGTGCATTCCTCGGATTTTCTTATGCGCCAGAGGATGATGACATCTTG | | |
| | GATTGTTAGAAGAGAAGGGCCTGTGAAACTACTGAGCCCAGCTGGTATTAGTAAGGAATTACCTTCA | | |
| | GCTGCTAGGAAGAGCGACTCAAACTAACAATGGCTT | | |
| | ORF Start: ATG at 220 | | ORF Stop: TAG at 1414 |
| | SEQ ID NO 140 | 398 aa | MW at 44552.5 kD |

| | |
|---|---|
| NOV33a, | MQGLLTSGRKPSGGGRCTGRGGWRGQWCLKPWMCGADPPTPTLSCLLLPVPPELPDHCYRNNSSPAG |
| CG149463-01 | |
| Protein Sequence | TPSPQPSRANGNINLGPSANPNAQPTDFDFLKVIGKGNYGKVLLAKRKSDGAFYAVKVLQKKSILKR |
| | KEQSHIMAERSVLLKNVRHPFLVGLRYSFQTPEKLYFVLDYVNGGELFFHLQRERRFLEPRARFYAA |
| | EVASAIGYLHSLNIIYRDLKPENILLDCQYLAPEVLRKEFYDRAVDWWCLGAVLYEMLHGLPPFYSQ |
| | DVSQMYENILHQPLQIPGGRTVAACDLLQSLLHKDQRQRLGSKADFLEIKNHVFFSPINWDDLYHKR |
| | LTPPFNPNVTGPADLKHFDPEFTQEAVSKSIGCTPDTVASSSGASSAFLGFSYAPEDDDILDC |

Further analysis of the NOV33a protein yielded the following properties shown in Table 33B.

TABLE 33B
Protein Sequence Properties NOV33a

| | |
|---|---|
| PSort analysis: | 0.4500 probability located in cytoplasm; |
| | 0.2677 probability located in microbody (peroxisome); |
| | 0.1859 probability located in lysosome (lumen); |
| | 0.1000 probability located in mitochondrial |
| | matrix space |
| SignalP analysis: | No Known Signal Sequence Predicted |

A search of the NOV33a protein against the Geneseq database, a proprietary database that contains sequences published in patents and patent publication, yielded several homologous proteins shown in Table 33C.

TABLE 33C
Geneseq Results for NOV33a

| | | NOV33a | Identities/ | |
|---|---|---|---|---|
| | | Residues/ | Similarities for | |
| Geneseq | Protein/Organism/Length | Match | the Matched | |
| Identifier | [Patent #, Date] | Residues | Region | Expect Value |
| AAY95276 | Human serum and | 1 . . . 398 | 398/427 (93%) | 0.0 |
| | glucocorticoid-induced | 1 . . . 427 | 398/427 (93%) | |
| | protein kinase 2-beta - Homo | | | |
| | sapiens, 427 aa. | | | |
| | [WO200035946-A1, | | | |
| | 22-JUN-2000] | | | |
| AAM25594 | Human protein sequence | 53 . . . 398 | 346/375 (92%) | 0.0 |
| | SEQ ID NO: 1109 - Homo | 8 . . . 382 | 346/375 (92%) | |
| | sapiens, 382 aa. | | | |
| | [WO200153455-A2, | | | |
| | 26-JUL-2001] | | | |
| AAE22765 | Human serum and | 61 . . . 398 | 338/367 (92%) | 0.0 |
| | glucocoticoid-induced | 1 . . . 367 | 338/367 (92%) | |
| | protein kinase, SGK2-alpha - | | | |
| | Homo sapiens, 367 aa. | | | |
| | [WO200224947-A2, | | | |
| | 28-MAR-2002] | | | |
| AAB65708 | Novel protein kinase, SEQ | 61 . . . 398 | 337/367 (91%) | 0.0 |
| | ID NO: 237 - Homo sapiens, | 1 . . . 367 | 338/367 (91%) | |
| | 367 aa. [WO200073469-A2, | | | |
| | 07-DEC-2000] | | | |
| AAB65615 | Novel protein kinase, SEQ | 184 . . . 398 | 215/244 (88%) | e−122 |
| | ID NO: 141 - Mus musculus, | 1 . . . 244 | 215/244 (88%) | |
| | 244 aa. [WO200073469-A2, | | | |
| | 07-DEC-2000] | | | |

In a BLAST search of public sequence datbases, the NOV33a protein was found to have homology to the proteins shown in the BLASTP data in Table 33D.

TABLE 33D
Public BLASTP Results for NOV33a

| | | NOV33a | Identities/ | |
|---|---|---|---|---|
| Protein | | Residues/ | Similarities for | |
| Accession | | Match | the Matched | |
| Number | Protein/Organism/Length | Residues | Portion | Expect Value |
| Q9HBY8 | Protein kinase - Homo sapiens | 1 . . . 398 | 398/427 (93%) | 0.0 |
| | (Human), 427 aa. | 1 . . . 427 | 398/427 (93%) | |
| Q9UKG6 | Protein kinase (DJ138B7.2) | 61 . . . 398 | 338/367 (92%) | 0.0 |
| | (Serum/glucocorticoid | 1 . . . 367 | 338/367 (92%) | |
| | regulated kinase 2) (Similar to | | | |
| | serum/glucocorticoid | | | |
| | regulated kinase 2) - Homo | | | |
| | sapiens (Human), 367 aa. | | | |
| Q8R0P6 | Serum/glucocorticoid | 61 . . . 397 | 317/366 (86%) | 0.0 |
| | regulated kinase 2 - Mus | 1 . . . 365 | 326/366 (88%) | |
| | musculus (Mouse), 366 aa. | | | |
| O73927 | S-sgk2 - Squalus acanthias | 70 . . . 396 | 235/359 (65%) | e−133 |
| | (Spiny dogfish), 594 aa. | 236 . . . 594 | 277/359 (76%) | |
| O73926 | S-sgk1 - Squalus acanthias | 61 . . . 396 | 239/374 (63%) | e−132 |
| | (Spiny dogfish), 433 aa. | 60 . . . 433 | 282/374 (74%) | |

PFam analysis predicts that the NOV33a protein contains the domains shown in the Table 33E.

TABLE 33E
Domain Analysis of NOV33a

| | | Identities/ | |
|---|---|---|---|
| Pfam | NOV33a | Similarities | Expect |
| Domain | Match Region | for the Matched Region | Value |
| pkinase | 95 . . . 228 | 54/135 (40%) | 5e−39 |
| | | 116/135 (86%) | |
| pkinase | 231 . . . 323 | 35/128 (27%) | 1.5e−21 |
| | | 69/128 (54%) | |
| pkinase₁₃C | 324 . . . 393 | 25/73 (34%) | 3.1e−15 |
| | | 47/73 (64%) | |

### Example 34

The NOV34 clone was analyzed, and the nucleotide and encoded polypeptide sequences are shown in Table 34A.

TABLE 34A
NOV34 Sequence Analysis

| | SEQ ID NO: 141 | | 2152 bp |
|---|---|---|---|
| NOV34a, | GGGGGGCCTGAGCCTCTCCGCCGGCGCAGGCTCTGCTCGCGCCAGCTCGCTCCCGCAGCCATGCCCA | | |
| CG149536-01 | | | |
| DNA Sequence | CCACCATCGAGCGGGAGTTCGAAGAGTTGGATACTCAGCGTCCCTGGCAGCCGCTGTACTTGGAAAT | | |
| | TCGAAATGAGTCCCATGACTATCCTCATAGAGTGGCCAAGTTTCCAGAAAACAGAAATCGAAACAGA | | |
| | TACAGAGATGTAAGCCCATATGATCACAGTCGTGTTAAACTGCAAAATGCTGAGAATGATTATATTA | | |
| | ATGCCAGTTTAGTTGACATAGAAGAGGCACAAAGGAGTTACATCTTAACACAGGGACCACTTCCTAA | | |
| | CACATGCTGCCATTTCTGGCTTATGGTTTGGCAGCAGAAGACCAAAGCAGTTGTCATGCTGAACCGC | | |
| | ATTGTGGAGAGAGAATCGAGTGGTGAAACCAGAACAATATCTCACTTTCATTATACTACCTGGCCAG | | |
| | ATTTTGGAGTCCCTGAATCACCAGCTTCATTTCTCAATTTCTTGTTTAAAGTGAGAGAATCTGGCTC | | |
| | CTTGAACCCTCACCATGGGCCTGCGGTGATCCACTGTAGTGCAGGCATTGGGCGCTCTGGCACCTTC | | |
| | TCTCTGGTAGACACTTGTCTTGTTTTGATCGAAAAAGGAGATGATATTAACATAAAACAAGTGTTAC | | |
| | TGAACATGAGAAAATACCGAATGGGTCTTATTCAGACCCCAGATCAACTGAGATTCTCATACATGGC | | |
| | TATAATAGAAGGAGCAAAATGTATAAAGGGAGATTCTAGTATACAGAAACGATGGAAAGAACTTTCT | | |
| | AAGGAAGACTTATCTCCTGCCTTTGATCATTCACCAAACAAAATAATGACTGAAAAATACAATGGGA | | |
| | ACAGAATAGGTCTAGAAGAAGAAAAACTGACAGGTGACCGATGTACACGACTTTCCTCTAAAATGCA | | |
| | AGATACAATGGAGGAGAACAGTGAGAGTGCTCTACGGAAACGTATTCGAGAGGACAGAAAGGCCACC | | |
| | ACAGCTCAGAAGGTGCAGCAGATGAAACAGAGGCTAAATGAGAATGAACCAGAAAAAGAAAAGTGGT | | |
| | TATATTGGCAACCTATTCTCACTAAGATGGGGTTTATGTCAGTCATTTTCGTTGGCGCTTTTGTTGG | | |
| | CTGGAGACTGTTTTTTCAGCAAAATGCCCTATAAACAATTAATTTTGCCCAGCAAGCTTCTGCACTA | | |
| | GTAACTGACAGTGCTACATTAATCATAGGGGTTTGTCTGCAGCAAACGCCTCATATCCCAAAAACGC | | |
| | TGCAGTAGAATAGACATCAACCAGATAAGTGATATTTACAGTCACAAGCCCAACATCTCAGGACTCT | | |
| | TGACTGCAGGTTCCTCTGAACCCCAAACTGTAAATGGCTGTCTAAAATAAAGACATTCATGTTTGTT | | |
| | AAAAACTGGTAAATTTTGCAACTGTATTCATACATGTCAAACACAGTATTTCACCTGACCAACATTG | | |
| | AGATATCCTTTATCACAGGATTTGTTTTTGGAGGCTATCTGGATTTTAACCTGCACTTGATATAAGC | | |
| | AATAAATATTGTGGTTTTATCTACGTTATTGGAAAGAAAATGACATTTAAATAATGTGTGTAATGTA | | |
| | TAATGTACTATTGACATGGGCATCAACACTTTTATTCTTAAGCATTTCAGGGTAAATATATTTTATA | | |
| | AGTATCTATTTAATCTTTTGTAGTTAACTGTACTTTTTAAGAGCTCAATTTGAAAAATCTGTTACTA | | |
| | AAAAAAAAAATTGTATGTCGATTGAATTGTACTGGATACATTTTCCATTTTTCTAAAAAGAAGTTTG | | |
| | ATATGAGCAGTTAGAAGTTGGAATAAGCAATTTCTACTATATATTGCATTTCTTTTATGTTTTACAC | | |
| | TTTTCCCCATTTTAAAAAGAAAAGCAAACAAAGAAACAAAAGTTTTTCCTAAAAATATCTTTGAAGG | | |
| | AAAATTCTCCTTACTGGGATAGTCAGGTAAACAGTTGGTCAAGACTTTGTAAAGAAATTGGTTTCTG | | |
| | TAAATCCCATTATTGATATGTTTATTTTTCATGAAAATTTCAATGTAGTTGGGGTAGATTATGATTT | | |
| | AGGAAGCAAAAGTAAGAAGCAGCATTTTATGATTCATAATTTCAGTTTACTACACTGAAGTTTTGAA | | |
| | GTAAACCC | | |
| | ORF Start: ATG at 61 | | ORF Stop: TAA at 1171 |
| | SEQ ID NO: 142 | 370 aa | MW at 43248.9 kD |

| | |
|---|---|
| NOV34a, | MPTTIEREFEELDTQRRWQPLYLEIRNESHDYPHRVAKFPENRNRNRYRDVSPYDHSRVKLQNAEND |
| CG149536-01 | |
| Protein Sequence | YINASLVDIEEAQRSYILTQGPLPNTCCHFWLMVWQQKTKAVVMLNRIVERESSGETRTISHFHYTT |
| | WPDFGVPESPASFLNFLFKVRESGSLNPDHGPAVIHCSAGIGRSGTFSLVDTCLVLMEKGDDINIKQ |
| | VLLNMRKYRMGLIQTPDQLRFSYMAIIEGAKCIKGDSSIQKRWKELSKEDLSPAFDHSPNKIMTEKY |
| | NGNRIGLEEEKLTGDRCTGLSSKMQDTMEENSESALRKRIREDRKATTAQKVQQMKQRLNENERKRK |
| | RWLYWQPILTKMGFMSVILVGAFVGWRLFFQQNAL |

Further analysis of the NOV34a protein yielded the following properties shown in Table 34B.

TABLE 34B
Protein Sequence Properties NOV34a

| | |
|---|---|
| PSort analysis: | 0.8500 probability located in endoplasmic |
| | reticulum (membrane); 0.4400 probability located in |
| | plasma membrane; 0.3000 probability located in |
| | nucleus; 0.1000 probability located in mitochondrial |
| | inner membrane |
| SignalP analysis: | No Known Signal Sequence Predicted |

A search of the NOV34a protein against the Geneseq database, a proprietary database that contains sequences published in patents and patent publication, yielded several homologous proteins shown in Table 34C.

TABLE 34C
Geneseq Results for NOV34a

| | | NOV34a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAR14114 | Non-receptor linked protein | 1 . . . 370 | 368/415 (88%) | 0.0 |
| | tyrosine phosphatase - Homo | 1 . . . 415 | 369/415 (88%) | |
| | sapiens, 415 aa. | | | |
| | [WO9113989-A, | | | |
| | 19-SEP-1991] | | | |
| AAU91293 | Human NOV8 protein - | 1 . . . 370 | 337/415 (81%) | 0.0 |
| | Homo sapiens, 415 aa. | 1 . . . 415 | 345/415 (82%) | |
| | [WO200216600-A2, | | | |
| | 28-FEB-2002] | | | |
| ABP41882 | Human ovarian antigen | 24 . . . 336 | 312/358 (87%) | e−178 |
| | HOCPJ87, SEQ ID NO:3014 - | 5 . . . 362 | 313/358 (87%) | |
| | Homo sapiens, 368 aa. | | | |
| | [WO200200677-A 1, | | | |
| | 03-JAN-2002] | | | |
| AAM25250 | Human protein sequence | 116 . . . 269 | 137/154 (88%) | 1e−77 |
| | SEQ ID NO:765 - Homo | 14 . . . 167 | 145/154 (93%) | |
| | sapiens, 168 aa. | | | |
| | [WO200153455-A2, | | | |
| | 26-JUL-2001] | | | |
| AAB56662 | Human prostate cancer | 1 . . . 124 | 123/124 (99%) | 1e−69 |
| | antigen protein sequence | 29 . . . 152 | 124/124 (99%) | |
| | SEQ ID NO:1240 - Homo | | | |
| | sapiens, 180 aa. | | | |
| | [WO200055174-A1, | | | |
| | 21-SEP-2000] | | | |

In a BLAST search of public sequence datbases, the NOV34a protein was found to have homology to the proteins shown in the BLASTP data in Table 34D.

TABLE 34D
Public BLASTP Results for NOV34a

| | | NOV34a | Identities/ | |
|---|---|---|---|---|
| Protein | | Residues/ | Similarities for | |
| Accession | | Match | the Matched | |
| Number | Protein/Organism/Length | Residues | Portion | Expect Value |
| P17706 | Protein-tyrosine phosphatase, | 1 . . . 370 | 369/415 (88%) | 0.0 |
| | non-receptor type 2 (EC | 1 . . . 415 | 370/415 (88%) | |
| | 3.1.3.48) (T - cell | | | |
| | protein-tyrosine phosphatase) | | | |
| | (TCPTP) - Homo sapiens | | | |
| | (Human), 415 aa. | | | |
| A33899 | protein-tyrosine-phosphatase | 1 . . . 370 | 368/415 (88%) | 0.0 |
| | (EC 3.1.3.48), nonreceptor type | 1 . . . 415 | 369/415 (88%) | |
| | 2 - human, 415 aa. | | | |
| A60345 | protein-tyrosine-phosphatase | 1 . . . 336 | 334/381 (87%) | 0.0 |
| | (EC 3.1.3.48) 11A - human, | 1 . . . 381 | 335/381 (87%) | |
| | 387 aa. | | | |
| Q922E7 | Protein tyrosine phosphatase, | 1 . . . 365 | 323/410 (78%) | 0.0 |
| | non-receptor type 2 - Mus | 1 . . . 405 | 338/410 (81%) | |
| | musculus (Mouse), 406 aa. | | | |
| Q06180 | Protein-tyrosine phosphatase, | l . . . 336 | 298/381 (78%) | e−168 |
| | non-receptor type 2 (EC | 1 . . . 376 | 312/381 (81%) | |
| | 3.1.3.48) (Protein-tyrosine | | | |
| | phosphatase PTP-2)(MPTP) - | | | |
| | Mus musculus (Mouse), 382 | | | |
| | aa. | | | |

PFam analysis predicts that the NOV34a protein contains the domains shown in the Table 34E.

TABLE 34E
Domain Analysis of NOV34a

| | | Identities/ | |
|---|---|---|---|
| | | Similarities | |
| | | for the Matched | Expect |
| Pfam Domain | NOV34a Match Region | Region | Value |
| Y₁₃phosphatase | 42 . . . 229 | 99/272 (36%) | 5.5e−88 |
| | | 163/272 (60%) | |

### Example 35

The NOV35 clone was analyzed, and the nucleotide and encoded polypeptide sequences are shown in Table 35A.

TABLE 35A
NOV35 Sequence Analysis

| | SEQ ID NO: 143 | 908 bp | |
|---|---|---|---|
| NOV35a, | CCCTTCTACCCAGAGGGTGAATGGGTATCTTTCCCGGAATAATCCTAATTTTTCTAAGGGTGAAGTT | | |
| CG149964-01 | | | |
| DNA Sequence | TGCAACCGCGGCCGTGACTGTAAGCGGACACCAGAAAAGTACCACTGTAAGTCATGAGATGTCTGGT | | |
| | CTGAATTGGAAACCCTTTGTATATGGCGGCCTTGCCTCTATCGTGGCTGAGTTTGGGACTTTCCCTG | | |
| | TGGACCTTACCAAAACACGACTTCAGGTTCAAGGCCAAAGCATTGATGCCCGTTTCAAAGAGATAAA | | |
| | ATATAGAGGCATGTTCCATGCGCTGTTTCGCATCTGTAAAGAGGAAGGTGTATTGGCTCTCTATTCA | | |
| | GGAATTGCTCCTGCGTTGCTAAGACAAGCATCATATGGCACCATTAAAATTGGGATTTACCAAAGCT | | |
| | TGAAGCGCTTATTCGTAGAACGTTTAGAAGATGAAACTCTTTTAATTAATATGATCTGTGGGGTAGT | | |
| | GTCAGGAGTGATATCTTCCACTATAGCCAATCCCACCCATGTTCTAAAGATTCGAATGCAGGCTCAA | | |
| | GGAAGCTTGTTCCAAGGGAGCATGATTGGAAGCTTTATCGATATATACCAACAAGAAGGCACCAGGG | | |
| | GTCTGTGGAGGGGTGTGGTTCCAACTGCTCACCGTGCTGCCATCGTTGTAGGAGTAGAGCTACCAGT | | |
| | CTATGATATTACTAAGAAGCATTTAATATTGTCAGGAATGATGGGACATGTGGATCTCTATAAGGGC | | |
| | ACTGTTGATGGTATTTTAAACATGTGGAAACATGAGGGCTTTTTTGCACTCTATAAAGGATTTTGGC | | |
| | CAAACTGGCTTCGGCTTGGACCCTGGAACATCATTTTTTTTATTACATACGACCAGGTAAAGAGGCT | | |
| | TCAAATCTAAGAACTGAATTATATGTGAGCCCAGCAC | | |
| | ORF Start: ATG at 21 | | ORF Stop: TAA at 879 |
| | SEQ ID NO: 144 | 286 aa | MW at 32043.5 kD |

| NOV35a, | MGIFPGIILIFLRVKFATAAVTVSGHQKSTTVSHEMSGLNWKPFVYGGLASIVAEFGTFPVDLTKTR | |
|---|---|---|
| CG149964-01 | | |
| Protein Sequence | LQVQGQSIDARFKEIKYRGMFHALFRICKEEGVLALYSGIAPALLRQASYGTIKIGIYQSLKRLFVE | |
| Protein Sequence | | |
| | RLEDETLLINMICGVVSGVISSTIANPTDVLKIRMQAQCSLFQGSMIGSFIDIYQQEGTRGLWRGVV | |
| | PTAQRAATVVGVELPVYDITKKHLILSGMMGHVDLYKGTVDGILKMWKHEGFFALYKGFWPNWLRLG | |
| | PWNIIFFITYEQVKRLQI | |
| | SEQ ID NO: 145 | 871 bp |

| | |
|---|---|
| NOV35b, | CACCGGATCCACCATGGGTATCTTTCCCGGAATAATCCTAATTTTTCTAAGGGTGAAGTTTGCAACG |
| 309326356 DNA | |
| Sequence | GCGGCCGTGATTCACCAGAAAAGTACCACTGTAAGTCATGAGATGTCTGGTCTGAATTGGAAACCCT |
| Sequence | |
| | TTGTATATGGCGGCCTTGCCTCTATCGTGGCTGAGTTTGGGACTTTCCCTGTGGACCTTACCAAAAC |
| | ACGACTTCAGGTTCAAGCCCAAAGCATTGATGCCCGTTTCAAAGAGATAAAATATAGAGGGATGTTC |
| | CATGCGCTGTTTCGCATCTGTAAAGACGAAGGTGTATTCGCTCTCTATTCAGGAATTGCTCCTGCGT |
| | TGCTAAGACAAGCATCATATGGCACCATTAAAATTGGGATTTACCAAAGCTTGAAGCGCTTATTCGT |
| | AGAACGTTTAGAAGATGAAACTCTTTTAATTAATATGATCTGTGGGGTAGTGTCAGGAGTGATATCT |
| | TCCACTATAGCCAATCCCACCGATGTTCTAAAGATTCGAATGCAGGCTCAAGGAAGCTTGTTCCAAG |
| | GGAGCATGATTCGAAGCTTTATCGATATATACCAACAAGAAGGCACCAGGGGTCTGTGGAGGGGTGT |
| | GGTTCCAACTGCTCAGCGTGCTCCCATCGTTGTAGGAGTAGAGCTACCAGTCTATGATATTACTAAG |
| | AACCATTTAATATTGTCAGGAATGATGGGACATGTGGATCTCTATAAGGGCACTGTTGATGGTATTT |
| | TAAAGATGTGGAAACATGAGGGCTTTTTTGCACTCTATAAAGGATTTTGGCCAAACTGGCTTCGGCT |
| | TGGACCCTGGAACATCATTTTTTTTATTACATACGAGCAGGTAAAGAGGCTTCAAATCGTCGACGGC |

| | ORF Start: at 2 | | ORF Stop: end of sequence |
|---|---|---|---|
| | SEQ ID NO:146 | 290 aa | MW at 32429.9 kD |
| NOV35b, | TGSTMGIFPGIILIFLRVKFATAAVIHQKSTTVSHEMSGLNWKPFVYGGLASTVAEFGTFPVDLTKT | | |
| 309326356 | | | |
| Protein Sequence | RLQVQGQSIDARFKEIKYRGMFHALFRICKEEGVLALYSGIAPALLRQASYGTIKIGIYQSLKRLFV | | |
| | ERLEDETLLINMICCVVSGVISSTIANPTDVLKIRMQAQGSLFQGSMIGSFIDIYQQEGTRGLWRGV | | |
| | VPTAQRAAIVVGVELPVYDITKKHLILSGMMGHVDLYKGTVDGILKMWKHEGFFALYKGFWPNWLRL | | |
| | GPWNIIFFITYEQVKRLQIVDG | | |
| | SEQ ID NO: 147 | 811 bp | |
| NOV35c, | CACCGGATCCGCCGTGATTCACCAGAAAAGTACCACTGTAAGTCATGAGATGTCTGGTCTGAATTGG | | |
| 309326444 DNA | | | |
| Sequence | AAACCCTTTCTATATGGCGGCCTTGCCTCTATCGTGGCTGAGTTTGGGACTTTCCCTGTGGACCTTA | | |
| | CCAAAACACCACTTCAGGTTCAAGGCCAAAGCATTGATGCCCGTTTCAAAGAGATAAAATATAGAGG | | |
| | GATGTTCCATGCGCTGTTTCGCATCTGTAAAGAGGAAGGTGTATTGGCTCTCTATTCAGGAATTGCT | | |
| | CCTGCGTTGCTAAGACAAGCATCATATGGCACCATTAAAATTGGGATTTACCAAAGCTTGAAGCGCT | | |
| | TATTCGTAGAACGTTTAGAAGATGAAACTCTTTTAATTAATATGATCTGTGGGGTAGTCTCAGGAGT | | |
| | GATATCTTCCACTATAGCCAATCCCACCGATGTTCTAAGATTCGAATGCAGGCTCAAGGAAGCCTTG | | |
| | TTCCAAGGGAGCATGATTGGAAGCTTTATCGATATATACCAACAAGAAGGCACCAGGGGTCTGTGGA | | |
| | GGGGTGTGGTTCCAACTGCTCAGCGTGCTGCCATCGTTGTAGGAGTAGAGCTACCAGTCTATGATAT | | |
| | TACTAAGAAGCATTTAATATTGTCAGGAATGATGGGACATGTGGATCTCTATAAGGGCACTGTTGAT | | |
| | GGTATTTTAAAGATGTGGAAACATGAGGGCTTTTTTGCACTCTATAAAGGATTTTGGCCAAACTGGC | | |
| | TTCGGCTTGGACCCTGGAACATCATTTTTTTTATTACATACGAGCAGGTAAAGAGGCTTCAAATCGT | | |
| | CGACGGC | | |
| | ORF Start: at 2 | | ORF Stop: end of sequence |
| | SEQ ID NO: 148 | 270 aa | MW at 30239.1 kD |
| NOV35c, | TGSAVIHQKSTTVSHEMSGLNWKPFVYGGLASIVAEFGTFPVDLTKTRLQVQGQSIDARFKEIKYRG | | |
| 309326444 | | | |
| Protein Sequence | MFHALFRTCKEEGVLALYSGIAPALLRQASYGTIKIGIYQSLKRLFVERLEDETLLINMICGVVSGV | | |
| | ISSTIANPTDVLKIRMQAQGSLFQGSMIGSFIDIYQQEGTRGLWRGVVPTAQRAAIVVGVELPVYDI | | |
| | TKKHLILSGMMGHVDLYKGTVDGILKMWKHEGFFALYKGFWPNWLRLGPWNIIFFITYEQVKRLQIV | | |
| | DG | | |
| | SEQ ID NO: 149 | 761 bp | |
| NOV35d, | CACCGGATCCCTGAATTGCAAACCCTTTGTATATGGCGGCCTTGCCTCTATCGTGGCTGAGTTTGGG | | |
| 309326473 DNA | | | |
| Sequence | ACTTTCCCTGTGGACCTTACCAAAACACGACTTCAGGTTCAAGGCCAAAGCATTGATGCCCGTTTCA | | |
| | AAGAGATAAAATATAGAGGGATGTTCCATGCGCTGTTTCGCATCTGTAAAGAGGAAGGTGTATTGGC | | |
| | TCTCTATTCAGGAATTGCTCCTGCGTTGCTAAGACAAGCATCATATGGCACCATTAAAATTGGGATT | | |
| | TACCAAAGCTTGAAGCGCTTATTCGTAGAACGTTTAGAAGATGAAACTCTTTTAATTAATATGATCT | | |
| | GTGGGGTAGTGTCAGGAGTGATATCTTCCACTATAGCCAATCCCACCGATGTTCTAAAGATTCGAAT | | |
| | GCAGGCTCAAGGAAGCTTGTTCCAAGGGAGCATGATTGGAAGCTTTATCGATATATACCAACAAGAA | | |
| | GGCACCAGGGGTCTGTGGAGGGGTGTGGTTCCAACTGCTCAGCGTGCTGCCATCGTTGTAGCAGTAG | | |
| | AGCTACCAGTCTATGATATTACTAAGAAGCATTTAATATTGTCAGGAATGATGGGACATGTGGATCT | | |
| | CTATAAGGGCACTGTTGATGGTATTTTAAAGATGTGGAAACATGAGGGCTTTTTTGCACTCTATAAA | | |
| | GGATTTTGGCCAAACTGGCTTCGGCTTGGACCCTGGAACATCATTTTTTTTATTACATACGAGCAGG | | |
| | TAAAGAGGCTTCAAATCGTCGACG | | |
| | ORF Start: at 2 | | ORF Stop: end of sequence |
| | SEQ ID NO: 150 | 254 aa | MW at 28488.2 kD |
| NOV35d, | TGSLNWKPFVYGGLASIVAEFGTFPVDLTKTRLQVQGQSIDARFKEIKYRGMFHALFRICKEEGVLA | | |
| 309326473 | | | |
| Protein Sequence | LYSGIAPALLRQASYGTIKIGIYQSLKRLFVERLEDETLLINMICGVVSGVISSTIANPTDVLKTRM | | |
| | QAQGSLFQGSMIGSEIDIYQQEGTRGLWRGVVPTAQRAAIVVGVELPVYDITKKHLILSGMMGHVDL | | |
| | YKGTVDGILKMWKHEGFFALYKGFWPNWLRLGPWNIIFFITYEQVKRLQIVDX | | |
| | SEQ ID NO: 151 | 1019 bp | |
| NOV35e, | CTACCCAGAGGGTGAATGGGTATCTTTCCCGGAATAATCCTAATTTTTCTAAGGGTGAAGTTTGCAA | | |
| CG149964-02 | | | |
| DNA Sequence | CGGCGGCCGTGACTGTAAGCGGACACCAGAAAAGTACCACTGTAAGTCATGAGATGTCTGGTCTGAA | | |
| | TTGGAAACCCTTTGTATATGGCGGCCTTGCCTCTATCGTGGCTGAGTTTGGGACTTTCCCTGTGGAC | | |
| | CTTACCAAAACACGACTTCAGGTTCAAGGCCAAAGCATTGATGCCCGTTTCAAAGAGATAAAATATA | | |
| | GAGGGATGTTCCATGCGCTGTTTCGCATCTGTAAAGAGGAAGGTGTATTGGCTCTCTATTCAGGAAT | | |
| | TGCTCCTGCGTTGCTAAGACAAGCATCATATGGCACCATTAAAATTGGGATTTACCAAAGCTTGAAG | | |
| | CGCTTATTCGTAGAACGTTTAGAAGATGAAACTCTTTTAATTAATATGATCTGTGGGGTAGTGTCAG | | |
| | GAGTGATATCTTCCACTATAGCCAATCCCACCGATGTTCTAAAGATTCGAATGCAGGCTCAAGGAAG | | |
| | CTTGTTCCAAGGGAGCATGATTGGAAGCTTTATCGATATATACCAGCAAGAAGGCACCAGGGGTCTG | | |
| | TGGAGGGGTGTGGTTCCAACTGCTCAGCGTGCTGCCATCGTTGTAGGAGTAGAGCTACCAGTCTATG | | |
| | ATATTACTAAGAAGCATTTAATATTGTCAGGAATGATGGGCGATACAATTTTAACTCACTTCGTTTC | | |
| | CAGCTTTACATGTGGTTTGGCTGGGGCTCTGGCCTCCAACCCGGTTGATGTGGTTCGAACTCGCATG | | |
| | ATGAACCAGAGGGCAATCGTGGGACATGTGGATCTCTATAAGGGCACTGTTGATGGTATTTTAAAGA | | |
| | TGTGGAAACATCAGCGCTTTTTTGCACTCTATAAAGGATTTTGGCCAAACTGGCTTCGGCTTGGACC | | |
| | CTGGAACATCATTTTTTTTATTACATACGAGCAGGTAAAGAGGCTTCAAATCTAAGAACTGAATTAT | | |
| | ATGTGAGCCCAGCC | | |
| | ORF Start: ATG at 16 | | ORF Stop: TAA at 991 |
| | SEQ ID NO: 152 | 325 aa | MW at 36175.2 kD |

| | |
|---|---|
| NOV35e, | MGIFPGIILIFLRVKFATAAVTVSGHQKSTTVSHEMSGLNWKPFVYGGLASIVAEFGTFPVDLTKTR |
| CG149964-02 | |
| Protein Sequence | LQVQGQSIDARFKEIKYRGMFHALFRICKEEGVLALYSGIAPALLRQASYGTIKIGIYQSLKRLFVE |
| | RLEDETLLINMICGVVSGVISSTIANPTDVLKIRMQAQGSLFQGSMTGSFIDIYQQEGTRGLWRGVV |
| | PTAQRAAIVVGVELPVYDITKKHLILSGMMGDTILTHFVSSFTCGLAGALASNPVDVVRTRMMNQRA |
| | IVGHVDLYKGTVDGILKMWKHEGFFALYKGFWPNWLRLGPWNIIFFITYEQVKRLQI |

Sequence comparison of the above protein sequences yields the following sequence relationships shown in Table 35B.

TABLE 35B
Comparison of NOV35a against NOV35b through NOV35e.

| Protein | NOV35a Residues/ | Identities/ |
|---|---|---|
| Sequence | Match Residues | Similarities for the Matched Region |
| NOV35b | 1 . . . 286 | 282/286 (98%) |
| | 5 . . . 287 | 282/286 (98%) |
| NOV35c | 26 . . . 286 | 261/261 (100%) |
| | 7 . . . 267 | 261/261 (100%) |
| NOV35d | 39 . . . 286 | 248/248 (100%) |
| | 4 . . . 251 | 248/248 (100%) |
| NOV35e | 1 . . . 286 | 286/325 (88%) |
| | 1 . . . 325 | 286/325 (88%) |

Further analysis of the NOV35a protein yielded the following properties shown in Table 35C.

TABLE 35C
Protein Sequence Properties NOV35a

| | |
|---|---|
| PSort analysis: | 0.4600 probability located in plasma membrane; |
| | 0.2648 probability located in microbody (peroxisome); |
| | 0.1000 probability located in endoplasmic reticulum |
| | (membrane); 0.1000 probability located in |
| | endoplasmic reticulum (lumen) |
| SignalP analysis: | Cleavage site between residues 20 and 21 |

A search of the NOV35a protein against the Geneseq database, a proprietary database that contains sequences published in patents and patent publication, yielded several homologous proteins shown in Table 35D.

TABLE 35D
Geneseq Results for NOV35a

| | | NOV35a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAY94665 | Human uncoupling protein | 1 . . . 286 | 284/325 (87%) | e−158 |
| | (UCP5) amino acid sequence - | 1 . . . 325 | 285/325 (87%) | |
| | Homo sapiens, 325 aa. | | | |
| | [WO200032624-A2, 08-JUN-2000] | | | |
| ABG33878 | Human secreted protein | 1 . . . 286 | 284/334 (85%) | e−155 |
| | encoded by gene 16 - Homo | 1 . . . 334 | 285/334 (85%) | |
| | sapiens, 334 aa. | | | |
| | [WO200226931-A2, 04-APR-2002] | | | |
| AAE06056 | Human gene 16 encoded | 1 . . . 286 | 284/334 (85%) | e−155 |
| | secreted protein HMIAP86, | 1 . . . 334 | 285/334 (85%) | |
| | SEQ ID NO:118 - Homo | | | |
| | sapiens, 334 aa. | | | |
| | [WO200151504-A1, 19-JUL-2001] | | | |
| AAY87079 | Human secreted protein | 1 . . . 286 | 284/334 (85%) | e−155 |
| | sequence SEQ ID NO:118 - | 1 . . . 334 | 285/334 (85%) | |
| | Homo sapiens, 335 aa. | | | |
| | [WO200004140-A1, 27-JAN-2000] | | | |
| AAY94666 | Human uncoupling protein | 1 . . . 286 | 281/325 (86%) | e−154 |
| | isoform hUCP5S amino acid | 1 . . . 322 | 282/325 (86%) | |
| | sequence - Homo sapiens, | | | |
| | 322 aa. [WO200032624-A2, | | | |
| | 08-JUN-2000] | | | |

In a BLAST search of public sequence datbases, the NOV35a protein was found to have homology to the proteins shown in the BLASTP data in Table 35E.

TABLE 35E
Public BLASTP Results for NOV35a

| | | NOV35a | Identities/ | |
|---|---|---|---|---|
| Protein | | Residues/ | Similarities for | |
| Accession | | Match | the Matched | |
| Number | Protein/Organism/Length | Residues | Portion | Expect Value |
| O95258 | Brain mitochondrial carrier | 1 . . . 286 | 284/325 (87%) | e−157 |
| | protein-1 (BMCP-1) | 1 . . . 325 | 285/325 (87%) | |
| | (Mitochondrial uncoupling | | | |
| | protein 5)(UCP 5)(Solute | | | |
| | carrier family 25, member 14) - | | | |
| | Homo sapiens (Human), 325 | | | |
| | aa. | | | |
| Q9Z2B2 | Brain mitochondrial carrier | 1 . . . 286 | 276/325 (84%) | e−154 |
| | protein-1 (BMCP-1) | 1 . . . 325 | 283/325 (86%) | |
| | (Mitochondrial uncoupling | | | |
| | protein 5)(UCP 5)(Solute | | | |
| | carrier family 25, member 14) - | | | |
| | Mus musculus (Mouse), 325 | | | |
| | aa. | | | |
| Q9EP88 | Brain mitochondrial carrier | 1 . . . 286 | 274/325 (84%) | e−153 |
| | protein BMCP1 (Brain | 1 . . . 325 | 282/325 (86%) | |
| | mitochondrial carrier | | | |
| | protein-1) - Rattus norvegicus | | | |
| | (Rat), 325 aa. | | | |
| Q9JMH0 | Brain mitochondrial carrier | 1 . . . 286 | 271/325 (83%) | e−149 |
| | protein-1 - Rattus norvegicus | 1 . . . 322 | 279/325 (85%) | |
| | (Rat), 322 aa. | | | |
| Q8R206 | Similar to RIKEN cDNA | 36 . . . 232 | 160/197 (81%) | 1e−87 |
| | 4933433D23 gene - Mus | 1 . . . 197 | 176/197 (89%) | |
| | musculus (Mouse), 210 aa. | | | |

PFam analysis predicts that the NOV35a protein contains the domains shown in the Table 35F.

TABLE 35F
Domain Analysis of NOV35a

| | | Identities/ | |
|---|---|---|---|
| Pfam | | Similarities | Expect |
| Domain | NOV35a Match Region | for the Matched Region | Value |
| mito_carr | 39 . . . 138 | 39/126 (31%) | 5.7e−24 |
| | | 78/126 (62%) | |
| mito_carr | 140 . . . 231 | 29/125 (23%) | 4.4e−27 |
| | | 76/125 (61%) | |
| mito_carr | 233 . . . 286 | 24/125 (19%) | 0.0072 |
| | | 46/125 (37%) | |

### Example 36

The NOV36 clone was analyzed, and the nucleotide and encoded polypeptide sequences are shown in Table 36A.

TABLE 36A
NOV36 Sequence Analysis

| | SEQ ID NO: 153 | 1144 bp | |
|---|---|---|---|
| NOV36a, | CGCGGGGCGCGCGGCGCGGGGCGGCCTGGCCGGCGGCGGCGGCGGCATGAAGGTCACGTCGCTCGAC | | |
| CG150306-01 | | | |
| DNA Sequence | GGGCGCCAGCTGCGCAAGATGCTCCGCAAGGAGGCGGCGGCGCGCTGCGTGGTGCTCGACTGCCGGC | | |
| | CCTATCTGGCCTTCGCTGCCTCGAACGTGCGCGGCTCGCTCAACGTCAACCTCAACTCGGTGGTGCT | | |
| | GGACCAGGGCAGCCGCCACTGGCAGAACCTGCGAGAGGAGAGCCCCGCGCGTGTCGTCCTCACCTCG | | |
| | CTACTCGCTTGCCTACCCGCCGGCCCGCGGGTCTACTTCCTCAAAGGGGGATATGAGACTTTCTACT | | |
| | CGGAATATCCTGAGTGTTGCGTGGATGTAAAACCCATTTCACAAGAGAAGATTGAGAGTGAGAGAGC | | |
| | CCTCATCAGCCAGTGTGGAAAACCAGTGGTAAATGTCAGCTACAGGCCAGCTTATCACCAGGGTGGC | | |
| | CCAGTTGAAATCCTTCCCTTCCTCTACCTTCGAAGTGCCTACCATGCATCCAAGTGCGAGTTCCTCG | | |
| | CCAACTTGCACATCACAGCCCTCCTGAATGTCTCCCGACGGACCTCCGACGCCTGCATGACCCACCT | | |
| | ACACTACAAATGGATCCCTGTGGAAGACAGCCACACGGCTGACATTAGCTCCCACTTTCAAGAAGCA | | |
| | ATAGACTTCATTGACTGTGTCAGGGAAAAGGGAGGCAAGGTCCTGGTCCACTGTGAGGCTGGGATCT | | |
| | CCCGTTCACCCACCATCTGCATGGCTTACCTTATGAAGACCAAGCAGTTCCGCCTGAAGGAGGCCTT | | |
| | CGATTACATCAAGCAGAGGAGGAGCATGGTCTCGCCCAACTTTGGCTTCATGGGCCAGCTCCTGCAG | | |
| | TACGAATCTGAGATCCTGCCCTCCACGCCCAACCCCCAGCCTCCCTCCTGCCAAGGGGAGGCAGCAG | | |
| | GCTCTTCACTGATAGGCCATTTGCAGACACTGAGCCCTGACATGCAGGGTGCCTACTGCACATTCCC | | |
| | TGCCTCGGTGCTGGCACCGGTGCCTACCCACTCAACAGTCTCAGAGCTCAGCAGAAGCCCTGTGGCA | | |
| | ACGGCCACATCCTGCTAAAACTGGGATGGAGGAATCGGCCCAGCCCCAAGAGCAACTGTGATTTTTG | | |
| | TTTTT | | |
| | ORF Start: ATG at 47 | | ORF Stop: TAA at 1088 |
| | SEQ ID NO: 154 | 347 aa | MW at 38362.6 kD |

| | |
|---|---|
| NOV36a, | MKVTSLDGRQLRKMLRKEAAARCVVLDCRPYLAFAASNVRGSLNVNLNSVVLDQGSRHWQKLREESA |
| CG150306-01 | |
| Protein Sequence | ARVVLTSLLACLPAGPRVYFLKGGYETFYSEYPECCVDVKPISQEKIESERALISQCGKPVVNVSYR |
| | PAYDQGGPVEILPFLYLGSAYHASKCEFLANLHITALLNVSRRTSEACMTHLHYKWIPVEDSHTADI |
| | SSHFQEAIDFIDCVREKGGKVLVHCEAGISRSPTICMAYLMKTKQFRLKEAFDYIKQRRSMVSPNFG |
| | FMGQLLQYESEILPSTPNPQPPSCQGEAAGSSLIGHLQTLSPDMQGAYCTFPASVLAPVPTHSTVSE |
| | LSRSPVATATSC |

Further analysis of the NOV36a protein yielded the following properties shown in Table 36B.

TABLE 36B
Protein Sequence Properties NOV36a

| | |
|---|---|
| PSort analysis: | 0.4811 probability located in mitochondrial matrix |
| | space; 0.4500 probability located in cytoplasm; |
| | 0.1892 probability located in mitochondrial inner |
| | membrane; 0.1892 probability located in mitochondrial |
| | intermembrane space |
| SignalP analysis: | No Known Signal Sequence Predicted |

A search of the NOV36a protein against the Geneseq database, a proprietary database that contains sequences published in patents and patent publication, yielded several homologous proteins shown in Table 36C.

TABLE 36C
Geneseq Results for NOV36a

| | | NOV36a | Identities/ | |
|---|---|---|---|---|
| | | Residues/ | Similarities | |
| Geneseq | Protein/Organism/Length | Match | for the | Expect |
| Identifier | [Patent #, Date] | Residues | Matched Region | Value |
| ABB07842 | Amino acid sequence of | 1 . . . 347 | 347/384 (90%) | 0.0 |
| | protein identified by | 1 . . . 384 | 347/384 (90%) | |
| | Swissprot Accn No. Q16690 - | | | |
| | Homo sapiens, 384 aa. | | | |
| | [WO200220732-A2, | | | |
| | 14 MAR. 2002] | | | |
| AAB66440 | Human MAP-kinase | 116 . . . 286 | 171/171 (100%) | 1e−97 |
| | phosphatase MKP-5 - Homo | 1 . . . 171 | 171/171 (100%) | |
| | sapiens, 171 aa. | | | |
| | [WO200102582-A1, | | | |
| | 11 JAN. 2001] | | | |
| AAE06784 | Human dual-specificity | 116 . . . 286 | 171/171 (100%) | 1e−97 |
| | phosphatase (DSP) protein, | 1 . . . 171 | 171/171 (100%) | |
| | MKP-5 - Homo sapiens, 171 | | | |
| | aa. [WO200157221-A2, | | | |
| | 09 AUG. 2001] | | | |
| AAR63602 | MAP-kinase-phosphatase | 1 . . . 347 | 168/388 (43%) | 5e−72 |
| | CL100 - Homo sapiens, 367 | 3 . . . 367 | 220/388 (56%) | |
| | aa. [WO9423039-A, | | | |
| | 13 OCT. 1994] | | | |
| AAU84270 | Human endometrial cancer | 1 . . . 347 | 167/388 (43%) | 1e−70 |
| | related protein, DUSP1 - | 3 . . . 367 | 219/388 (56%) | |
| | Homo sapiens, 367 aa. | | | |
| | [WO200209573-A2, | | | |
| | 07 FEB. 2002] | | | |

In a BLAST search of public sequence datbases, the NOV36a protein was found to have homology to the proteins shown in the BLASTP data in Table 36D.

TABLE 36D
Public BLASTP Results for NOV36a

| | | | Identities/ | |
|---|---|---|---|---|
| Protein | | | Similarities for | |
| Accession | | NOV36a Residues/ | the Matched | Expect |
| Number | Protein/Organism/Length | Match Residues | Portion | Value |
| Q16690 | Dual specificity protein | 1 . . . 347 | 347/384 (90%) | 0.0 |
| | phosphatase 5 (EC 3.1.3.48) | 1 . . . 384 | 347/384 (90%) | |
| | (EC 3.1.3.16) (Dual | | | |
| | specificity protein | | | |
| | phosphatase hVH3) - Homo | | | |
| | sapiens (Human), 384 aa. | | | |
| O54838 | Dual specificity protein | 1 . . . 347 | 320/384 (83%) | 0.0 |
| | phosphatase 5 (EC 3.1.3.48) | 1 . . . 384 | 336/384 (87%) | |
| | (EC 3.1.3.16) (MAP-kinase | | | |
| | phosphatase CPG21) - Rattus | | | |
| | norvegicus (Rat), 384 aa. | | | |
| Q90W58 | MAP kinase phosphatase | 13 . . . 347 | 164/378 (43%) | 9e−72 |
| | XCL100 (beta) protein - | 15 . . . 369 | 217/378 (57%) | |
| | Xenopus laevis (African | | | |
| | clawed frog), 369 aa. | | | |
| P28562 | Dual specificity protein | 1 . . . 347 | 167/388 (43%) | 3e−70 |
| | phosphatase 1 (EC 3.1.3.48) | 3 . . . 367 | 219/388 (56%) | |
| | (EC 3.1.3.16) (MAP kinase | | | |
| | phosphatase-1) (MKP-1) | | | |
| | (Protein-tyrosine phosphatase | | | |
| | CL100) (Dual specificity | | | |
| | protein phosphatase hVH1) - | | | |
| | Homo sapiens (Human), 367 | | | |
| | aa. | | | |
| O42253 | Dual specificity protein | 15 . . . 344 | 166/366 (45%) | 1e−68 |
| | phosphatase 1 (EC 3.1.3.48) | 4 . . . 353 | 213/366 (57%) | |
| | (EC 3.1.3.16) (MAP kinase | | | |
| | phosphatase-1) (MPK-1) | | | |
| | (MAP kinase phosphatase-1) - | | | |
| | Gallus gallus (Chicken), 353 | | | |
| | aa (fragment). | | | |

PFam analysis predicts that the NOV36a protein contains the domains shown in the Table 36E.

TABLE 36E
Domain Analysis of NOV36a

| | | Identities/ | |
|---|---|---|---|
| | NOV36a | Similarities | |
| | Match | for the Matched | Expect |
| Pfam Domain | Region | Region | Value |
| Rhodanese | 7 . . . 98 | 23/134 (17%) | 0.0052 |
| | | 66/134 (49%) | |
| DSPc | 141 . . . 279 | 76/172 (44%) | 1.8e−70 |
| | | 132/172 (77%) | |
| Y_phosphatase | 44 . . . 279 | 39/336 (12%) | 0.54 |
| | | 144/336 (43%) | |

### Example 37

The NOV37 clone was analyzed, and the nucleotide and encoded polypeptide sequences are shown in Table 37A.

TABLE 37A
NOV37 Sequence Analysis

| | |
|---|---|
| SEQ ID NO: 155 | 2277 bp |

| NOV37a, | CGCGTTCTCGGCTCCCGCCGGGGTCCCCCGCGGCTGTCGCCGCCGCCTACGCCGCTCCCTCCGCCTT | | |
|---|---|---|---|
| CG150510-01 | | | |
| DNA Sequence | CCTGCCCCGCGTCGGGCCGGGCGCCACCTCCCCCCTGCCTCCCTCTCCGCTCTGGTCATTTAGGAAA | | |
| | TCGTAAATCATGTGAAGATGGGACTCTTGGTATTTGTGCGCAATCTGCTGCTAGCCCTCTGCCTCTT3 | | |
| | TCTGGTACTCGGATTTTTGTATTATTCTGCGTGGAAGCTACACTTACTCCAGTGGGAGGACGACTCC | | |
| | AGTAAGTATAGTCACTCTAGCTCACCCCAGGAGAAGCCTGTTGCAGATTCACTGGTTCTTTCCTTTG | | |
| | ACTCCGCTGGACAAACACTAGGCTCAGAGTATGATCGGTTGGGCTTCCTCCTGAATCTGGACTCTAA | | |
| | ACTGCCTGCTGAATTAGCCACCAAGTACGCAAACTTTTCAGAGGGAGCTTGCAAGCCTGGCTATGCT | | |
| | TCAGCCTTGATGACGGCCATCTTCCCCCGGTTCTCCAAGCCAGCACCCATGTTCCTGGATGACTCCT | | |
| | TTCGCAAGTGGGCTAGAATCCGGGAGTTCGTGCCGCCTTTTGGGATCAAAGGTCAAGACAATCTGAT | | |
| | CAAAGCCATCTTGTCAGTCACCAAAGAGTACCGCCTGACCCCTGCCTTGGACAGCCTCCGCTGCCGC | | |
| | CGCTGCATCATCGTGGGCAATGGAGGCGTTCTTGCCAACAAGTCTCTGGGGTCACGAATTGACGACT | | |
| | ATGACATTGTGGTGAGACTGAATTCAGCACCAGTGAAAGGCTTTGAGAAGGACGTGGGCAGCAAAAC | | |
| | GACACTGCGCATCACCTACCCCCAGGGCGCCATGCAGCGGCCTGAGCAGTACGAGCGCGATTCTCTC | | |
| | TTTGTCCTCGCCGGCTTCAAGTGGCAGGACTTTAAGTGGTTGAAATACATCGTCTACAAGGAGAGAG | | |
| | TGAGTGCATCGGATGGCTTCTGCAAATCTGTGGCCACTCGAGTGCCCAAGGAGCCCCCTGAGATTCG | | |
| | AATCCTCAACCCATATTTCATCCAGGAGGCCGCCTTCACCCTCATTGGCCTGCCCTTCAACAATGGC | | |
| | CTCATGGGCCGGGGGAACATCCCTACCCTTGGCAGTGTGGCAGTCACCATGGCACTACACGGCTGTG | | |
| | ACGAGGTGGCAGTCGCAGGATTTGGCTATGACATGAGCACACCCAACGCACCCCTGCACTACTATGA | | |
| | GACCGTTCGCATGGCAGCCATCAAAGAGTCCTGGACGCACAATATCCAGCGAGAGAAAGAGTTTCTG | | |
| | CGGAAGCTGGTGAAAGCTCGCGTCATCACTGATCTAAGCAGTGGCATCTGAGTGGGCCCAGCACATG | | |
| | GCCATAGAGGCCCAGGCACCACCAGGAGCAGCAGCCAGCACCACCTACACACGAGTCTTCAGACCCA | | |
| | GAGAAGGACGGTGCCAAGGGCCCCAGGGGCAGCAAGGCCTTGGTGGAGCAGCCAGAGCTGTGCCTGC | | |
| | TCAGCAGCCAGTCTCAGAGACCAGCACTCAGCCTCATTCAGCATGGGTCCTTGATGCCAGAGGGCCA | | |
| | GCAGGCTCCTGGCTGTGCCCAGCACGCCCAGCATGCAGGTGGTGGGACACTGGGCAGCAAGGCTGCT | | |
| | GCCGGAATCACTTCTCCAATCAGTGTTTGGTGTATTATCATTTTGTGAATTTGGGTAGGGGGGAGGG | | |
| | TAGGGATAATTTATTTTTAAATAAGGTTGGAGATGTCAAGTTGGGTTCACTTGCCATGCAGGAAGAG | | |
| | GCCCACTAGAGGGCCCATCAGGCAGTGTTACCTGTTAGCTCCCTGTGGGGCAGGAGTGCCAGGACCA | | |
| | GCCTGTACCTTGCTGTGGGGCTACAGGATGGTGGGCAGGATCTCAAGCCAGCCCCCTCCAGCTCATG | | |
| | ACACTGTTTGGCCTTTCTTGGGGAGAAGGCGGGGTATTCCCACTCACCAGCCCTAGCTGTCCCATGG | | |
| | GGAAACCCTGGAGCCATCCCTTCGGAGCCAACAAGACCGCCCCAGCGCTATAGCAGAAAGAACTTTA | | |
| | AAGCTCAGCAGGGTGACGCCCAGCTCCGCCTGCTGGGAAGAGCTCCCCTCCACAGCTGCAGCTGATC | | |
| | CATAGGACTACCGCAGGCCCGGACTCACCAACTTGCCACATGTTCTAGGTTTCAGCAACAAGACTGC | | |
| | CAGGTGGTTGGGTTCTGCCTTTAGCCTGGACCAAAGGGAAGTGAGGCCCAAGGAGCTTACCCAAGCT | | |
| | GTGGCAGCCGTCCCAGGCCACCCCCATGGAAGCAATAAAGCTCTTCCCTGTAAAAAAAAAAAAAAA | | |
| | ORF Start: ATG at 152 | | ORF Stop: TGA at 1322 |
| | SEQ ID NO: 156 | 390 aa | MW at 43785.1 kD |

| | |
|---|---|
| NOV37a, | MGLLVFVRNLLLALCLFLVLGFLYYSAWKLHLLQWEEDSSKYSHSSSPQEKPVADSVVLSFDSAGQT |
| CG150510-01 | |
| Protein Sequence | LGSEYDRLGFLLNLDSKLPAELATKYANFSEGACKPGYASALMTAIFPRFSKPAPMFLDDSFRKWAR |
| | IREFVPPFGIKGQDNLIKAILSVTKEYRLTPALDSLRCRRCIIVGNGGVLANKSLGSRIDDYDIVVR |
| | LNSAPVKGFEKDVGSKTTLRITYPEGAMQRPEQYERDSLFVLAGFKWQDEKWLKYIVYKERVSASDG |
| | FWKSVATRVPKEPPEIRILNPYFIQEAAFTLIGLPFNNGLMGRGNIPTLGSVAVTMALHGCDEVAVA |
| | GFGYDMSTPNAPLHYYETVRMAAIKESWTHNIQREKEFLRKLVKARVITDLSSGI |

Further analysis of the NOV37a protein yielded the following properties shown in Table 37B.

TABLE 37B
Protein Sequence Properties NOV37a

| | |
|---|---|
| PSort analysis: | 0.8200 probability located in outside; 0.2360 |
| | probability located in microbody (peroxisome); |
| | 0.1900 probability located in lysosome (lumen); |
| | 0.1000 probability located in endoplasmic |
| | reticulum (membrane) |
| SignalP analysis: | Cleavage site between residues 22 and 23 |

A search of the NOV37a protein against the Geneseq database, a proprietary database that contains sequences published in patents and patent publication, yielded several homologous proteins shown in Table 37C.

TABLE 37C
Geneseq Results for NOV37a

| | | | Identities/ | |
|---|---|---|---|---|
| | | | Similarities for | |
| Geneseq | Protein/Organism/Length | NOV37a Residues/ | the Matched | Expect |
| Identifier | [Patent #, Date] | Match Residues | Region | Value |
| AAY39960 | Human alpha2-3 sialate | 1 . . . 390 | 374/390 (95%) | 0.0 |
| | transferase protein sequence - | 1 . . . 375 | 375/390 (95%) | |
| | Homo sapiens, 375 aa. | | | |
| | [JP11253163-A, | | | |
| | 21 SEP. 1999] | | | |
| AAR65242 | Human ST3N | 1 . . . 390 | 374/390 (95%) | 0.0 |
| | sialyltransferase - Homo | 1 . . . 375 | 375/390 (95%) | |
| | sapiens, 375 aa. | | | |
| | [W09504816-A, | | | |
| | 16 FEB. 1995] | | | |
| AAR63217 | Human | 1 . . . 390 | 374/390 (95%) | 0.0 |
| | alpha-2, 3-sialyltransferase | 1 . . . 375 | 375/390 (95%) | |
| | (WM16) - Homo sapiens | | | |
| | (melanoma WM266-4 cells), | | | |
| | 375 aa. [WO9423021-A, | | | |
| | 13 OCT. 1994] | | | |
| AAR62808 | Alpha 2, 3-sialyl transferase - | 1 . . . 390 | 374/390 (95%) | 0.0 |
| | Homo sapiens, 375 aa. | 1 . . . 375 | 375/390 (95%) | |
| | [JP06277052-A, | | | |
| | 04 OCT. 1994] | | | |
| AAR41671 | Rat sialyltransferase - Rattus | 1 . . . 390 | 361/390 (92%) | 0.0 |
| | rattus, 374 aa. | 1 . . . 374 | 370/390 (94%) | |
| | [WO9318157-A, | | | |
| | 16 SEP. 1993] | | | |

In a BLAST search of public sequence datbases, the NOV37a protein was found to have homology to the proteins shown in the BLASTP data in Table 37D.

TABLE 37D
Public BLASTP Results for NOV37a

| | | | Identities/ | |
|---|---|---|---|---|
| Protein | | | Similarities for | |
| Accession | | NOV37a Residues/ | the Matched | Expect |
| Number | Protein/Organism/Length | Match Residues | Portion | Value |
| Q11203 | CMP-N-acetylneuraminate-beta-1,4- | 1 . . . 390 | 374/390 (95%) | 0.0 |
| | galactoside alpha-2,3-sialyltransferase | 1 . . . 375 | 375/390 (95%) | |
| | (EC 2.4.99.6) (N-acetyllactosaminide | | | |
| | alpha-2,3-sialyltransferase) (Gal | | | |
| | beta-1,3(4) GlcNAc alpha-2,3 | | | |
| | sialyltransferase) (ST3N) | | | |
| | (Sialyltransferase 6) - Homo sapiens | | | |
| | (Human), 375 aa. | | | |
| Q922X5 | Sialyltransferase (N-acetyllacosaminide | 1 . . . 390 | 361/390 (92%) | 0.0 |
| | alpha 2,3-sialyltransferase) - Mus | 1 . . . 374 | 371/390 (94%) | |
| | musculus (Mouse), 374 aa. | | | |
| Q9DBB6 | Sialyltransferase (N-acetyllacosaminide | 1 . . . 390 | 360/390 (92%) | 0.0 |
| | alpha 2,3- sialyltransferase) - Mus | 1 . . . 374 | 371/390 (94%) | |
| | musculus (Mouse), 374 aa. | | | |
| Q02734 | CMP-N-acetylneuraminate-beta-1,4- | 1 . . . 390 | 361/390 (92%) | 0.0 |
| | galactoside alpha-2,3- sialyltransferase | 1 . . . 374 | 370/390 (94%) | |
| | (EC 2.4.99.6) (N-acetyllactosaminide | | | |
| | alpha-2,3- sialyltransferase) (Gal | | | |
| | beta-1,3(4) GlcNAc alpha-2,3 | | | |
| | sialyltransferase) (ST3N) | | | |
| | (Sialyltransferase 6) - Rattus norvegicus | | | |
| | (Rat), 374 aa. | | | |
| P97325 | CMP-N-acetylneuraminate-beta-1,4- | 1 . . . 390 | 359/390 (92%) | 0.0 |
| | galactoside alpha-2,3- sialyltransferase | 1 . . . 374 | 370/390 (94%) | |
| | (EC 2.4.99.6) (N-acetyllactosaminide | | | |
| | alpha-2,3- sialyltransferase) (Gal | | | |
| | beta-1,3(4) GlcNAc alpha-2,3 | | | |
| | sialyltransferase) (ST3N) | | | |
| | (Sialyltransferase 6) - Mus musculus | | | |
| | (Mouse), 374 aa. | | | |

PFam analysis predicts that the NOV37a protein contains the domains shown in the Table 37E.

TABLE 37E
Domain Analysis of NOV37a

| | | Identities/ | |
|---|---|---|---|
| | NOV37a | Similarities | |
| | Match | for the | Expect |
| Pfam Domain | Region | Matched Region | Value |
| Glyco_transf_29 | 101 . . . 389 | 108/324 (33%) | 3.2e−116 |
| | | 270/324 (83%) | |

### Example 38

The NOV38 clone was analyzed, and the nucleotide and encoded polypeptide sequences are shown in Table 38A.

TABLE 38A
NOV38 Sequence Analysis

| | SEQ ID NO: 157 | 1976 bp | |
|---|---|---|---|
| NOV38a, | CCCTTATGAAGACGGGACATTTTGAAATAGTCACCATGCTGCTGGCAACCATGATTCTAGTGGACAT | | |
| CG150704-01 | | | |
| DNA Sequence | TTTCCAGGTGAAGGCTGAAGTGTTAGACATGGCACATAATGCATTTGATGATGAATACCTGAAATGT | | |
| | ACGGACAGGATGGAAATTAAATACGTTCCCCAACTGCTAAAGGAGGAAAAAGCAAGCCACCAGCAAT | | |
| | TAGATACTGTGTGGGAAAATGCAAAAGCCAAATGCGCAGCCCCAAAGACTCAAATCTTTCTCCCTAT | | |
| | GAATTTTAAGGATAACCATGGAATAGCCCTGATGGCATATATTTCCGAACCTCAAGAGCAAACTCCC | | |
| | TTTTACCATCTGTTCAGTGAAGCTGTCAAGATGGCTGGCCAATCTCGAGAAGATTATATCTATGGCT | | |
| | TCCAGTTCAAAGCTTTCCACTTTTACCTCACAAGAGCCCTGCAGTTGCTGAGAAAACCTTGTGAGGC | | |
| | CAGTTCCAAAACTGTGGTATATAGAACAAGCCACGGCACTTCATTTACATTTGGAGGGCTAAACCAA | | |
| | GCCAGGTTTGGCCATTTTACCTTGGCATATTCAGCCAAACCTCAGGCTGCTAATGACCAGCTCACTG | | |
| | TGTTATCCATCTACACATGCCTTGGAGTTGACATTCAAAATTTTCTTCATAAAGAAAGTGAAAGAAT | | |
| | TACTTTAATACCTCTGAATGAGGTTTTTCAAGTGTCACAGGAGGGGGCTGGCAATAACCTTATCCTT | | |
| | CAAAGCATAAACAAGACCTGCAGCCATTATGAGTGTGCATTTCTACGTGGACTAAAAACCGAAAACT | | |
| | GTATTGAGAACCTAGAATATTTTCAACCCATCTATGTCTACAACCCTGGTGAGAAAAACCAGAAGCT | | |
| | TCAAGACCATAGTGAGAAAAACTGGAAGCTTGAAGACCATGGTGAGAAAAACCACAAGCTTGAAGAC | | |
| | CATGCTCCAGGTCCAGTTCCTGTTCCAGGTCCCAAAAGCCATCCTTCTGCATCCTCGGGCAAACTGC | | |
| | TGCTTCCACAGTTTGGGATGGTCATCATTTTAATCAGTGTTTCTGCTATAAATCTCTTTGTTGCTCT | | |
| | GTAG | | |
| | ORF Start: ATG at 6 | | ORF Stop: TAG at 1074 |
| | SEQ NO: 158 | 356 aa | MW at 40311.7 kD |

| | |
|---|---|
| NOV38a, | MKTGHFEIVTMLLATMILVDIFQVKAEVLDMADNAFDDEYLKCTDRMEIKYVPQLLKEEKASHQQLD |
| CG150704-01 | |
| Protein Sequence | TVWENAKAKWAARKTQIFLPMNFKDNHGIALMAYISEAQEQTPFYHLFSEAVKMAGQSREDYIYGFQ |
| | FKAFHFYLTRALQLLRKPCEASSKTVVYRTSQGTSFTFGGLNQARFGHFTLAYSAKPQAANDQLTVL |
| | SIYTCLGVDIENFLDKESERITLIPLNEVFQVSQEGAGNNLILQSINKTCSHYECAPLGGLKTENCI |
| | ENLEYFQPIYVYNPGEKNQKLEDHSEKNWKLEDHGEKNQKLEDHAPGPVPVPGPKSHPSASSGKLLL |
| | PQFGMVIILISVSATMLFVAL |

Further analysis of the NOV38a protein yielded the following properties shown in Table 38B.

TABLE 38B
Protein Sequence Properties NOV38a

| | |
|---|---|
| PSort analysis: | 0.6850 probability located in endoplasmic reticulum |
| | (membrane); 0.6400 probability located in plasma |
| | membrane; 0.4600 probability located in Golgi body; |
| | 0.1000 probability located in endoplasmic |
| | reticulum (lumen) |
| SignalP analysis: | Cleavage site between residues 27 and 28 |

A search of the NOV38a protein against the Geneseq database, a proprietary database that contains sequences published in patents and patent publication, yielded several homologous proteins shown in Table 38C.

TABLE 38C
Geneseq Results for NOV38a

| | | | Identities/ | |
|---|---|---|---|---|
| | | | Similarities for | |
| Geneseq | Protein/Organism/Length | NOV38a Residues/ | the Matched | Expect |
| Identifier | [Patent #, Date] | Match Residues | Region | Value |
| AAR41876 | Human HT6 - Homo sapiens, | 29 . . . 256 | 82/238 (34%) | 1e−21 |
| | 230 aa. [DE4209216-A, | 7 . . . 227 | 120/238 (49%) | |
| | 23 SEP. 1993] | | | |
| AAW76806 | Human | 20 . . . 266 | 83/266 (31%) | 6e−21 |
| | ADP-ribosyltransferase | 31 . . . 287 | 123/266 (46%) | |
| | protein - Homo sapiens, 327 | | | |
| | aa. [US5834310-A, | | | |
| | 10 NOV. 1998] | | | |
| AAW76804 | Rabbit skeletal muscle | 8 . . . 259 | 88/282 (31%) | 1e−20 |
| | ADP-ribosyltransferase | 6 . . . 280 | 130/282 (45%) | |
| | protein - Oryctolagus | | | |
| | cuniculus, 327 aa. | | | |
| | [US5834310-A, | | | |
| | 10 NOV. 1998] | | | |
| AAR37572 | Rabbit skeletal muscle | 8 . . . 259 | 88/282 (31%) | 1e−20 |
| | ADP-ribosyltransferase - | 6 . . . 280 | 130/282 (45%) | |
| | Oryctolagus cuniculus, 327 | | | |
| | aa. [USN7985698-N, | | | |
| | 01 MAY 1993] | | | |
| ABB97573 | Novel human protein SEQ ID | 29 . . . 163 | 59/137 (43%) | 1e−20 |
| | NO: 841 - Homo sapiens, 229 | 29 . . . 161 | 76/137 (55%) | |
| | aa. [WO200222660-A2, | | | |
| | 21 MAR. 2002] | | | |

In a BLAST search of public sequence datbases, the NOV38a protein was found to have homology to the proteins shown in the BLASTP data in Table 38D.

TABLE 38D
Public BLASTP Results for NOV38a

| | | | Identities/ | |
|---|---|---|---|---|
| Protein | | | Similarities for | |
| Accession | | NOV38a Residues/ | the Matched | Expect |
| Number | Protein/Organism/Length | Match Residues | Portion | Value |
| S62906 | mono-ADP-ribosyltransferase - | 1 . . . 356 | 356/367 (97%) | 0.0 |
| | human, 367 aa. | 1 . . . 367 | 356/367 (97%) | |
| Q8WVJ7 | Hypothetical 42.7 kDa protein - | 1 . . . 356 | 355/378 (93%) | 0.0 |
| | Homo sapiens (Human), 378 | 1 . . . 378 | 355/378 (93%) | |
| | aa. | | | |
| Q13508 | Ecto-ADP-ribosyltransferase 3 | 1 . . . 356 | 355/389 (91%) | 0.0 |
| | precursor (EC 2.4.2.31) | 1 . . . 389 | 355/389 (91%) | |
| | (NAD(P)(+)-- arginine | | | |
| | ADP-ribosyltransferase 3) | | | |
| | (Mono(ADP-ribosyl)transferase | | | |
| | 3) - Homo sapiens (Human), | | | |
| | 389 aa. | | | |
| Q96HL1 | Unknown (protein for | 1 . . . 356 | 354/389 (91%) | 0.0 |
| | MGC: 14489) - Homo sapiens | 1 . . . 389 | 354/389 (91%) | |
| | (Human), 389 aa. | | | |
| Q9GKV6 | Hypothetical 38.2 kDa protein - | 31 . . . 356 | 300/338 (88%) | e−174 |
| | Macaca fascicularis (Crab | 1 . . . 338 | 312/338 (91%) | |
| | eating macaque) (Cynomolgus | | | |
| | monkey), 338 aa. | | | |

PFam analysis predicts that the NOV38a protein contains the domains shown in the Table 38E.

TABLE 38E
Domain Analysis of NOV38a

| | | Identities/ | |
|---|---|---|---|
| | NOV38a | Similarities | |
| Pfam | Match | for the | Expect |
| Domain | Region | Matched Region | Value |
| ART | 1 . . . 312 | 164/340 (48%) | 1.5e−200 |
| | | 312/340 (92%) | |

### Example 39

The NOV39 clone was analyzed, and the nucleotide and encoded polypeptide sequences are shown in Table 39A.

TABLE 39A
NOV39 Sequence Analysis

| | SEQ ID NO: 159 | 8350 bp | |
|---|---|---|---|
| NOV39a, | CAGGGAAAAGGGAACCTATGGAATGGTCATGGTGACTTTTGAGGTAGAGGGTGGCCCAAATCCCCCT | | |
| CG150799-01 | | | |
| DNA Sequence | GATGAAGATTTGAGTCCAGTTAAAGGAAATATCACCTTTCCCCCTGGCAGAGCAACAGAAATTTATA | | |
| | ACTTGACAGTACTCGATGACGAGGTACCAGAAAATGATGAAATATTTTTAATTCAACTGAAAAGTGT | | |
| | AGAAGGAGGAGCTGAGATTAACACCTCTAGGAATTCCATTGAGATCATCATTAAGAAAAATGATAGT | | |
| | CCCGTGAGATTCCTTCAGAGTATTTATTTGGTTCCTGAGGAAGACCACATACTCATAATTCCAGTAG | | |
| | TTCGTGGAAAGGACAACAATCGAAATCTCATTGGATCTGATGAATATGAGGTTTCAATCAGTTATGC | | |
| | TGTCACAACTGGGAATTCCACAGCACATGCCCAGCAAAATCTGGACTTCATTGATCTTCAGCCAAAC | | |
| | ACAACTGTTGTTTTTCCACCTTTTATTCATGAATCTCACTTGAAATTTCAAATAGTTGATGACACCA | | |
| | CACCGGAGATTGCTGAATCGTTTCACATTATGTTACTAAAAGATACCTTACAGGGAGATGCTGTGCT | | |
| | AATAAGCCCTTCTGTTGTACAAGTCACCATTAAGCCAAATGATAAACCTTATGGAGTCCTTTCATTC | | |
| | AACAGTGTTTTGTTTGAAAGGACAGTTATAATTGATCAAGATAGAATATCAAGATATGAAGAAATCA | | |
| | CAGTCGTTAGAAATGGAGGAACCCATGGGAATGTCTCTGCGAATTGGGTGTTGACACGGAACAGCAC | | |
| | TGATCCCTCACCAGTAACAGCACATATCAGACCGAGCTCTGGAGTTCTCCATTTTGCACAACGGCAG | | |
| | ATGTTGGCAACAATTCCTCTTACTGTGGTTGATGATGATCTTCCAGAAGAGGCAGAAGCTTATCTAC | | |
| | TTCAAATTCTGCCTCATACAATACGAGGACCTGCAGAAGTGAGCGAGCCAGCGGAGGATAGTGATGA | | |
| | TGTCTATGGCCTAATAACATTTTTTCCTATGGAAAACCAGAAGATTCAAAGCAGCCCAGGTGAACGA | | |
| | TACTTATCCTTGAGTTTTACAAGACTAGGAGGGACTAAAGGAGATGTGAGGTTGCTTTATTCTGTAC | | |
| | TTTACATTCCTGCTGGAGCTGTGGACCCCTTGCAAGCAAAAGAAGGCATCTTAAATATATCAAGGAG | | |
| | AAATGACCTCATTTTTCCAGAGCAAAAAACTCAAGTCACTACAAAATTACCAATAAGAAATGATGCA | | |
| | TTCTTTCAAAATGGAGCTCACTTTCTAGTACAGTTGCAAACTGTGGAGTTGTTAAACATAATTCCTC | | |
| | TAATCCCACCCATAAGCCCTAGATTTGGGGAAATCTGCAATATTTCTTTACTGGTTACTCCAGCCAT | | |
| | TGCAAATGGAGAAATTGGCTTTCTCAGCAATCTTCCAATTATTTTGCATGAACCAGAAGATTTTGCT | | |
| | GCTGAAGTGGTATACATTCCCTTACATCGGGATGGAACTGATGGCCAGGCTACTGTCTACTGGAGTT | | |
| | TGAAGCCCTCTGGCTTTAATTCAAAAGCAGTGACCCCCGATGATATAGGCCCCTTTAATGGCTCTGT | | |
| | TTTGTTTTTATCTGGGCAAAGTGACACAACAATCAACATTACTATCAAAGGTGATGACATACCGGAA | | |
| | ATGAATGAAACTGTAACACTTTCTCTAGACAGGGTTAACGTGGAAAACCAAGTGCTGAAATCTCGAT | | |
| | ATACTAGCCGTGACCTAATTATTTTGGAAAATGATGACCCTCGGGGAGTTTTTGAATTTTCTCCTGC | | |
| | TTCCAGAGGACCCTATGTTATAAAAGAAGGAGAATCTGTAGAGCTCCACATCATCCGATCAAGGGGG | | |
| | TCCCTTGTTAAGCAGTTTCTACACTACCGAGTAGAGCCAAGAGATAGCAATGAATTCTATGGAAACA | | |
| | CGGGAGTACTAGAATTTAAACCTGGAGAAAGGGAGATAGTGATCACCTTGCTAGCAAGATTCGATGG | | |
| | GATACCAGAGTTGGATGAACACTACTGGGTCGTCCTCAGCAGCCACGGAGAACGGGAAAGCAAGTTG | | |
| | CGAAGTGCCACCATTGTCAATATAACGATTCTGAAAAATGATGATCCTCATGGCATTATAGAATTTG | | |
| | TTTCTGATGGTCTAATTGTGATGATAAATGAAAGCAAAGGAGATGCTATCTATAGTGCTGTTTATGA | | |
| | TGTAGTAAGAAATCGAGGCAACTTTGGTGATGTTAGTGTATCATGGTTGGTTAGTCCAGACTTTACA | | |
| | CAAGATGTATTTCCTGTACAAGGGACTGTTGTCTTTGGAGATCACGAATTTTCAAAAAATATCACCA | | |
| | TTTACTCCCTTCCAGATGAGATTCCAGAAGAAATGGAAGAATTTACCGTTATCCTACTGAATGGCAC | | |
| | TGGAGGAGCTAAACTGGGAAATAGAACAACTGCAACTCTGAGGATTAGAAGAAATGATGACCCCATT | | |
| | TATTTTGCAGAACCTCGTGTAGTGAGGGTTCAGGAAGGTGAGACTGCCAACTTTACAGTTCTCAGAA | | |
| | ATGGATCTGTTGATGTGACTTGCATGGTCCAGTATGCTACCAAGGATGGGAAGGCTACTCCAAGAGA | | |
| | GAGAGATTTCATTCCTGTTGAAAAAGGAGAAACGCTCATTTTTGAGGTTGGAAGTAGACAGCAGAGC | | |
| | ATATCCATATTTGTTAATGAAGATGGTATCCCGGAAACAGATGAGCCCTTTTATATAATCCTCTTGA | | |
| | ATTCAACAGGTGATACAGTAGTATATCAATATGGAGTAGCTACAGTAATAATTGAAGCTAATGATGA | | |
| | CCCAAATGGCATTTTTTCTCTGGAGCCCATAGACAAAGCAGTGGAAGAAGGAAAGACTAATGCATTT | | |
| | TGGATTTTGAGGCACCGAGGATACTTTGGTAGTGTTTCTGTATCTTGGCAGCTCTTTCAGAATGATT | | |
| | CTGCTTTGCAGCCTGGGCAGGAGTTCTATGAAACTTCACGAACTGTTAACTTCATGGATGGAGAAGA | | |
| | AGCAAAACCAATCATTCTCCATGCTTTTCCAGATAAAATTCCTGAATTCAATGAATTTTATTTCCTA | | |
| | AAACTTGTAAACATTTCAGGTCCTCGGGGCCAGCTAGCAGAAACCAACCTCCAGGTGACACTAATGG | | |
| | TTCCATTCAATGATGATCCCTTTGGAGTTTTTATCTTGGATCCAGAGTGTTTAGAGAGAGAAGTGGC | | |
| | AGAAGATGTCCTGTCTGAAGATGATATGTCTTATATTACCAACTTCACCATTTTGACGCAGCAGGGT | | |
| | GTGTTTCGTGATGTACAACTGGGCTGGGAAATACTGTCCAGTGAGTTCCCTGCTGGTTTGCCACCAA | | |
| | TGATAGATTTTTTACTGGTTGGAATTTTCCCCACCACCGTGCATTTACAACAGCACATGCCGCGTCA | | |
| | CCACAGTGGAACGGATGCTTTGTACTTTACCGGACTAGAGGGTGCATTTGGGACTGTTAATCCAAAA | | |
| | TACCATCCCTCCAGGIATAATACAATTGCCAACTTTACATTCTCAGCTTGGGTAATGCCCAATGCCA | | |
| | ATACGAATGGATTCATTATAGCGAAGGATGACCGTAATGGAAGCATCTACTACGGGGTAAAAATACA | | |
| | AACAAACGAATCCCATGTGACACTTTCCCTTCATTATAAAACCTTGGGTTCCAATGCTACATACATT | | |
| | GCCAAGACAACAGTCATGAAATATTTAGAAGAAAGTGTTTGGCTTCATCTACTAATTATCCTGGAGG | | |
| | ATGGTATAATCGAATTCTACCTGGATGGAAATGCAATGCCCAGGGGAATCAAGAGTCTGAAAGGAGA | | |
| | AGCCATTACTGACGGTCCTGGGATACTGAGAATTGGAGCAGGGATAAATGGCAATGACAGATTTACA | | |
| | GGTCTGATGCAGGATGTGAGGTCCTATCAGCGGAAACTGACCCTTGAAGAAATTTATGAACTTCATG | | |
| | CCATGCCCGCAAAAAGTGATTTACACCCAATTTCTGGATATCTGGAGTTCAGACAGGGAGAAACTAA | | |
| | CAAATCATTCATTATTTCTGCAAGAGATGACAATGACGAGGAAGGAGAAGAATTATTCATTCTTAAA | | |
| | CTACTTTCTGTATATGGAGGAGCTCGTATTTCGGAAGAAAATACTACTGCAAGATTAACAATACAAA | | |
| | AAAGTGACAATGCAAATGGCTTGTTTCGTTTCACACGAGCTTGTATACCAGAGATTGCAGAGGAGGG | | |
| | ATCAACCATTTCTTGTGTGGTTGAGAGAACCAGAGGAGCTCTGGATTATGTGCATGTTTTTTACACC | | |
| | ATTTCACAGATTGAAACTGATGGCATTAATTACCTTGTTGATGACTTTGCTAATGCCAGTGGAACTA | | |
| | TTACATTCCTTCCTTGGCAGAGATCAGAGGTTCTGAATATATATGTTCTTGATGATGATATTCCTGA | | |
| | ACTTAATGAGTATTTCCGTGTGACATTGGTTTCTGCAATTCCTGGAGATGGGAAGCTAGGCTCAACT | | |
| | CCTACCAGTGGTGCAACCATAGATCCTGAAAAGGAAACGACTGATATCACCATCAAAGCTAGTGATC | | |
| | ATCCATATGGCTTGCTGCAGTTCTCCACAGGGCTCCCTCCTCAGCCTAACGACGCAATGACCCTGCC | | |
| | TGCAAGCAGCGTTCCACATATCACTGTGGAGGAGGAAGATGGAGAAATCACGTTATTGGTCATCCGT | | |
| | GCACACGGACTTCTGGCAAGGGTGACTGCGGAATTTAGAACAGTGTCCTTGACAGCATTCAGTCCTG | | |
| | AGGATTACCAGAATGTTGCTGGCACATTAGAATTTCAACCAGGAGAAAGATATAAATACATTTTCAT | | |
| | AAACATCACTGATAATTCTATTCCTGAACTGGAAAAATCTTTTAAAGTTGAGTTGTTAAACTTGGAA | | |
| | GGAGGAGTAGCTGAACTCTTTAGGGTTGATGGAAGTCGTAGTGCCAGTCTAGGAGTGGCTTCCCAAA | | |
| | TTCTACTGACAATTGCAGCCTCTGACCACGCTCATGGCGTATTTGAATTTAGCCCTGAGTCACTCTT | | |
| | TGTCAGTGGAACTGAACCAGAAGATGGGTATAGCACTGTTACATTAAATGTTATAAGACATCATGGA | | |
| | ACTCTGTCTCCAGTGACTTTGCATTGGAACATAGACTCTGATCCTGATGGTGATCTCGCCTTCACCT | | |
| | CTGGCAACATCACATTTGAGATTGGGCAGACGAGCGCCAATATCACTGTGGAGATATTGCCTGACGA | | |
| | AGACCCAGAACTCGATAAGGCATTCTCTGTGTCAGTCCTCAGTGTTTCCAGTGGTTCTTTGGGAGCT | | |
| | CATATTAATGCCACGTTAACAGTTTTGGCTAGTGATGATCCATATGGGATATTCATTTTTTCTGAGA | | |
| | AAAACAGACCTGTTAAAGTTGAGGAAGCAACCCAGAACATCACACTATCAATAATAAGGTTGAAAGG | | |
| | CCTCATGGGAAAAGTCCTTGTCTCATATGCAACACTAGATGATATGGAAAAACCACCTTATTTTCCA | | |
| | CCTAATTTAGCGAGACCAACTCAAGGAAGAGACTATATACCACCTTCTGGATTTGCTCTTTTTGGAG | | |
| | CTAATCAGAGTGAGGCAACAATAGCTATTTCAATTTTGGATGATGATGAGCCAGAAAGGTCCGAATC | | |
| | TGTCTTTATCGAACTACTCAACTCTACTTTAGTAGCGAAAGTACAGAGTCGTTCAATTCCAAATTCT | | |
| | CCACGTCTTGGGCCTAAGGTAGAAACTATTGCGCAACTAATTATCATTGCCAATGATGATGCATTTG | | |
| | GAACTCTTCAGCTCTCAGCACCAATTGTCCGAGTGGCAGAAAATCATGTTGGACCCATTATCAATGT | | |
| | GACTAGAACAGGAGGAGCATTTGCAGATGTCTCTGTGAAGTTTAAAGCTGTGCCAATAACTGCAATA | | |
| | GCTCGTGAAGATTATAGTATAGCTTCATCAGATGTGGTCTTGCTAGAAGGGGAAACCAGTAAAGCCG | | |
| | TGCCAATATATGTCATTAATGATATCTATCCTGAACTGGAAGAATCTTTTCTTGTCCAACTGATGAA | | |
| | TGAAACAACAGGAGGAGCCAGACTAGGGGCTTTAACAGAGGCAGTCATTATTATTGAGGCCTCTGAT | | |
| | GACCCCTATGGATTATTTGGTTTTCAGATTACTAAACTTATTGTAGAGGAACCTGAGTTTAACTCAG | | |
| | TGAAGGTAAACCTGCCAATAATTCGAAATTCTGGGACACTCGGCAATGTTACTGTTCAGTGGGTTGC | | |
| | CACCATTAATGGACAGCTTGCTACTGGCGACCTGCGAGTTGTCTCAGGTAATGTGACCTTTGCCCCT | | |
| | GGGGAAACCATTCAAACCTTGTTGTTAGAGGTCCTGGCTGACGACGTTCCGGAGATTGAAGAGGTTA | | |
| | TCCAAGTGCAACTAACTGATGCCTCTCGTGGAGGTACTATTGGGTTAGATCGAATTGCAAATATTAT | | |
| | TATTCCTGCCAATGATGATCCTTATGGTACAGTAGCCTTTGCTCAGATGGTTTATCGTGTTCAAGAG | | |
| | CCTCTGGAAAGAAGTTCCTGTGCTAATATAACTGTCAGGCGAAGCGGAGGGCACTTTGGTCGGCTGT | | |
| | TCTTGTTCTACAGTACTTCCGACATTCATGTAGTGGCTCTGGCAATGGAGGAAGGTCAAGATTTACT | | |
| | GTCCTACTATGAATCTCCAATTCAAGGGGTGCCTGACCCACTTTGGAGAACTTGGATGAATGTCTCT | | |
| | GCCGTCCCGGAGCCCCTGTATACCTGTGCCACTTTGTGCCTTAAGGAACAAGCTTGCTCAGCGTTTT | | |
| | CATTTTTCAGTGCTTCTGAGGGTCCCCAGTGTTTCTGGATGACATCATGGATCAGCCCACCTGTCAA | | |
| | CAATTCAGACTTCTGGACCTACAGGAAAAACATGACCAGGGTAGCATCTCTTTTTAGTGCTCAGGCT | | |
| | GTGGCTGGGAGTGACTATGAGCCTGTGACAAGGCAATGCGCCATAATGCAGGAAGGTGATGAATTCG | | |
| | CAAATCTCACAGTGTCTATTCTTCCTGATGATTTCCCAGAGATCGATGAGAGTTTTCTAATTTCTCT | | |
| | CCTTGAAGTTCACCTCATGAACATTTCAGCCAGTTTGAAAAATCAGCCAACCATAGGACAGCCAAAT | | |
| | ATTTCTACAGTTGTCATAGCACTAAATGGTGATGCCTTTGCAGTGTTTGTGATCTACAATATTACTC | | |
| | CCAATACTTCCGAAGATGGCTTATTTGTTGAAGTTCACGAGCAGCCCCAAACCTTGGTGCAGCTGAT | | |
| | CATACACAGGACAGGGGGCAGCTTAGGTCAAGTGCCAGTCGAATGGCGTCTTGTTGGTGCAACAGCT | | |
| | ACTGAAGGTTTAGATTTTATAGGTGCTGGAGAGATTCTGACCTTTGCTGAAGGTCAAACCAAAAAGA | | |
| | CAGTCATTTTAACCATCTTGGATGACTCTGAACCAGAGGATGACGAAAGTATCATAGTTAGTTTGCT | | |
| | GTACACTGAAGCTCGAAGTAGAATTTTGCCAAGCTCCGACACTGTTAGAGTGAACATTTTGCCCAAT | | |
| | GACAATGTGGCAGCAATTGTTAGCTTTCAGACAGCTTCCAGATCTGTCATAGGTCATGAACGAGAAA | | |
| | TTTTACAATTCCATGTGATAAGAACTTTCCCTGGTCGAGGAAATGTTACTGTTAACTGGAAAATTAT | | |
| | TGGCCAAAATCTAGAACTCAATTTTGCTAACTTTAGCGGACAACTTTTCTTTCCTGAGGGGTCGTTG | | |
| | AATACAACATTGTTTGTGCATTTGTTGGATGACAACATTCCTGAGGAGAAAGAAGTATACCAAGTCA | | |
| | TTCTGTATGATGTCAGGACACAAGGAGTTCCACCAGCCGGAATCGCCCTGCTTGATGCTCAAGGATA | | |
| | TGCACCTGTCCTCACAGTAGAACCCAGTGATGAACCACATGGAGTTTTAAATTTTGCTCTTTCATCA | | |
| | AGATTTGTCTTACTACAAGAGGCTAACATAACAATTCACCTTTTCATCAACAGAGAATTTGGATCTC | | |
| | TACGAGCTATCAATGTCACATATACCACGGTTCCTGGAATGCTGAGTCTGAAGAACCAAACACTACG | | |
| | AAACCTAGCAGAGCCAGAAGTTGATTTTGTCCCTATCATTCGCTTTCTGATTTTAGAAGAAGGGCAA | | |
| | ACAGCAGCAGCCATCAACATTACCATTCTTGAGGATGATGTACCAGAGCTAGAAGAATATTTCCTCG | | |
| | TGAATTTAACTTACGTTGGACTTACCATGCCTGCTTCAACTTCATTTCCTCCCACACTACGTATCAG | | |
| | GGGTTTCTTGTTTGTTTCTTTTTGCTCACTTCAAATGAAATGAAGAAACTTCATTTTTGAATCAGAA | | |
| | GTGATCATTGTCCTGTTTTGTTAATCTTACCTATGTGTTAAA | | |
| | ORF Start: ATG at 23 | | ORF Stop: TGA at 8282 |
| | SEQ ID NO: 160 | 2753 aa | MW at 301743.8 kD |
| NOV39a, | NVMVTFEVECGPNPPDEDLSPVKGNITFPPGRATVIYNLTVLDDEVPENDEIFLIQLKSVEGGAEIN | | |
| CG150799-01 | | | |
| Protein Sequence | TSRNSIEIIIKKNDSPVRFLQSIYLVPEEDHILIIPVVRGKDNNGNLIGSDEYEVSISYAVTTGNST | | |
| | AHAQQNLDFIDLQFNTTVVFPPFTHESHLKFQIVDDTTFEIAESFHIMLLKDTLQGDAVLISPSVVQ | | |
| | VTIKPNDKPYGVLSFNSVLFERTVIIDEDRISRYEEITVVRNGGTHCNVSANWVLTRNSTDPSPVTA | | |
| | DIRPSSGVLHFAQGQMLATIPLTVVDDDLPEEAEAYLLQILPHTIRGGAEVSEPAEDSDDVYGLITF | | |
| | FPMENQKIESSPGERYLSLSFTRLGGTKGDVRLLYSVLYIPAGAVDPLQAKEGILNISRRNDLIFPE | | |
| | QKTQVTTKLPIRNDAFFQNGAHFLVQLETVELLNIIPLIPPISPRFGEICNISLLVTPAIANGEIGF | | |
| | LSNLPIILHEPEDFAAEVVYIPLHRDGTDGQATVYWSLKPSGFNSKAVTPDDIGPFNGSVLFLSGQS | | |
| | DTTINITIKGDDIPEMNETVTLSLDRVNVENQVLKSGYTSRDLIILENDDPGGVFEFSPASRGPYVI | | |
| | KEGESVELHIIRSRGSLVKQFLHYRVEPRDSNEFYGNTCVLEFKPGEREIVITLLARLDGIPELDEH | | |
| | YWVVLSSHGERESKLGSATIVNITILKNDDPHGIIEFVSDGLIVMINESKGDAIYSAVYDVVRNRGN | | |
| | FGDVSVSWVVSPDFTQDVFPVQGTVVFGDQEFSKNITIYSLPDEIPEEMEEFTVILLNGTGGAKVGN | | |
| | RTTATLRIRRNDDPIYFAEPRVVRVQEGETANFTVLRNGSVDVTCMVQYATKDGEATARERDFIPVE | | |
| | KGETLIFEVGSRQQSISIFVNEDGIFETDEPFYIILLNSTGDTVVYQYGVATVIIEANDDPNGIFSL | | |
| | EPIDKAVEEGKTNAFWILRHRGYFGSVSVSWQLFQNDSALQPGQEFYETSGTVNFMDGEEAKPIILH | | |
| | AFPDKIPEFNEFYFLKLVNISGPGGQLAETNLQVTVMVPFNDDPFGVFILDPECLEREVAEDVLSED | | |
| | DMSYITNFTILRQQGVFGDVQLGWEILSSEFPAGLPPMIDFLLVGIFPTTVHLQQHMRRHHSGTDAL | | |
| | YFTGLEGAFGTVNPKYHPSRNNTIANFTFSAWVMPNANTNGFIIAKDDCNGSIYYGVKIQTNESHVT | | |
| | LSLHYKTLGSNATYIAKTTVMXYLEESVWLHLLIILEDGIIEFYLDGNAMPRGIKSLKGEAITDGPG | | |
| | ILRIGAGINGNDRFTGLMQDVRSYERKLTLEEIYELHAMPAKSDLHPISGYLEFRQGETNKSFIISA | | |
| | RDDNDEEGEELFILKLVSVYGGARISEENTTARLTIQKSDNANGLFGFTGACIPEIAEEGSTISCVV | | |
| | ERTRGALDYVHVFYTISQIETDGINYLVDDFANASGTITFLPWQRSEVLNIYVLDDDIPELNEYFRV | | |
| | TLVSAIPGDGKLGSTPTSGASIDPEKETTDTTIKASDHPYGLLQFSTGLPPQPKDAMTLPASSVPHI | | |
| | TVEEEDGEIRLLVIRAQGLLGRVTAEFRTVSLTAFSPEDYQNVAGTLEFQPGERYKYIFINITDNSI | | |
| | PELEKSFKVELLNLEGGVAELFRVDGSGSASLGVASQILVTIAASDHAHGVFEFSPESLFVSGTEPE | | |
| | DGYSTVTLNVIRHHCTLSPVTLEWNIDSDPDGDLAFTSGNITFEIGQTSANITVEILPDEDPELDKA | | |
| | FSVSVLSVSSGSLGAHINATLTVLASDDFYGIFIFSEKNRPVKVEEATQNITLSIIRLKGLMGKVLV | | |
| | SYATLDDMEKPPYFPPNLARATQGRDYIPASGFALFGANQSEATIAISILDDDEPERSESVFIELLN | | |
| | STLVAKVQSRSIPNSPRLGPKVETIAQLIIIANDDAFGTLQLSAPIVRVAENHVCPIINVTRTGGAE | | |
| | ADVSVKFKAVPITAIAGEDYSIASSDVVLLEGETSKAVPIYVINDIYPELEESFLVQLMNETTGGAR | | |
| | LGALTEAVIIIEASDDFYGLFGFQITKLIVEEPEFNSVKVNLPIIRNSGTLGNVTVQWVATINGQLA | | |
| | TGDLRVVSGNVTFAPGETIQTLLLEVLADDVFEIEEVIQVQLTDASGGGTIGLDRIANIIIPANDDP | | |
| | YGTVAFAQMVYRVQEPLERSSCANITVRRSGGHFGRLLLFYSTSDIDVVALAMEEGQDLLSYYESPI | | |
| | QGVPDPLWRTWMNVSAVGEPLYTCATLCLKEQACSAFSFFSASEGPQCFWMTSWISPAVNNSDFWTY | | |
| | RKNMTRVASLFSGQAVAGSDYEPVTRQWAIMQEGDEFANLTVSILPDDFPEMDESFLISLLEVHLNN | | |
| | ISASLKNQPTIGQPNISTVVIALNGDAFGVFVIYNISPNTSEDGLFVEVQEQPQTLVELMIHRTGGS | | |
| | LGQVAVEWRVVGGTATEGLDFIGAGEILTFAEGETKKTVILTILDDSEPEDDESIIVSLVYTEGGSR | | |
| | ILPSSDTVRVNILANDNVAGIVSFQTASRSVIGHEGEILQFHVIRTFPGRGNVTVNWKIIGQNLELN | | |
| | FANFSGQLFFPEGSLNTTLFVHLLDDNIPEEKEVYQVILYDVRTQGVPPAGIALLDAQGYAAVLTVE | | |
| | ASDEPHGVLNFALSSRFVLLGEANITIGLFINREFGSLGAINVTYTTVPGMLSLKNQTVGNLAEPEV | | |
| | DFVPIIGFLILEEGETAAAINITILEDDVPELEEYFLVNLTYVGLTMAASTSFPPRLGMRGFLFVSF | | |
| | ORF Start: ATG at 23 | | ORF Stop: TAA at 11537 |
| | SEQ ID NO: 162 | 3838 aa | MW at 421384.3 kD |
| NOV39b, | MVMVTFEVEGGPNPPDEDLSPVKGNITFPPGRATVIYNLTVLDDEVPENDEIFLIQLKSVEGGAEIN | | |
| CG150799-02 | | | |
| Protein Sequence | TSRNSIEIIIKKNDSPVRFLQSIYLVPEEDHILIIPVVRGKDNNGNLICSDEYEVSISYAVTTGNST | | |
| | AHAQQNLDFIDLQPNTTVVFPPFIHESHLKFQIVDDTTPEIAESFHIMLLKDTLQGDAVLISPSVVQ | | |
| | VTIKPNDKFYGVLSFNSVLFERTVIIDEDRISRYEEITVVRNGGTHGNVSANWVLTRNSTDPSPVTA | | |
| | DIRPSSGVLHFAQGQMLATIFLTVVDDDLPEEAEAYLLQILPHTIRGGAEVSEPAEDSDDVYGLTTF | | |
| | FPMENQKIESSFGERYLSLSFTRLGGTKGDVRLLYSVLYIPAGAVDPLQAKEGILNISRRNDLIFPE | | |
| | QKTQVTTKLPIRNDAFFQNCAHFLVQLETVELLNIIPLIPPISPRFGEICNISLLVTPAIANGEIGF | | |
| | LSNLPIILHEPEDFAAEVVYIPLHRDGTDGQATVYWSLKPSGFNSKAVTPDDIGPFNGSVLFLSGQS | | |
| | DTTINITIKGDDIPEMNETVTLSLDRVNVENQVLKSGYTSRDLIILENDDFGGVFEFSPASRGPYVI | | |
| | KEGESVELHIIRSRGSLVKQFLHYRVEPRDSNEFYGNTGVLEFKFGEREIVITLLARLDGIPELDEH | | |
| | YWVVLSSHGERESKLGSATIVNITILKNDDPHGIIEFVSDGLIVMINESKGDAIYSAVYDVVRNRGN | | |
| | FGDVSVSWVVSPDFTQDVFPVQGTVVFGDQEFSKNITIYSLPDEIPEEMEEFTVILLNGTGGAKVGN | | |
| | RTTATLRIRRNDDPIYFAEPRVVRVQEGETANFTVLRNGSVDVTCMVQYATKDGKATARERDFIPVE | | |
| | KGETLIFEVGSRQQSISIFVNEDGIPETDEPFYIILLNSTGDTVVYQYGVATVIIEANDDPNGIFSL | | |
| | EPIDKAVEEGKTNAFWILRHRGYFGSVSVSWQLFQNDSALQFGQEFYETSGTVNFHDGEEAKPIILE | | |
| | AFFDKIPEFNEFYFLKLVNISGPGCQLAETNLQVTVMVPFNDDPFGVFILDPECLEREVAEDVLSED | | |
| | DMSYITNFTILRQQGVFGDVQLGWETLSSEFPAGLPPMIDFLLVGIFPTTVHLQQHMRRHHSGTDAL | | |
| | YFTGLEGAFGTVNPKYHPSRNNTIANFTFSAWVMPNANTNGFIIAKDDGNGSIYYGVKIQTNESHVT | | |
| | LSLHYKTLGSNATYIAKTTVMKYLEESVWLHLLIILEDGIIEFYLDGNAMPRGIKSLKGEAITDGPG | | |
| | ILRIGAGINGNDRFTGLMQDVRSYERKLTLEEIYELHAMPAKSDLHPISGYLEFRQGETNKSFIISA | | |
| | RDDNDEEGEELFILKLVSVYGGARISEENTTARLTIQKSDNANGLFGFTGACIPEIAEEGSTISCVV | | |
| | ERTRGALDYVHVFYTISQIETDGINYLVDDFANASGTITFLPWQRSEVLNIYVLDDDIPELNEYFRV | | |
| | TLVSAIPGDGKLGSTPTSGASIDPEKETTDITIKASDHPYGLLQFSTGLPFQPKDAMTLPASSVPHI | | |
| | TVEEEDGEIRLLVIRAQGLLGRVTAEFRTVSLTAFSPEDYQNVAGTLEFQPGERYKYIFINITDNSI | | |
| | PELEKSFKVELLNLEGGVAELFRVDGSGSASLGVASQILVTIAASDHAHGVFEFSPESLFVSGTEPE | | |
| | DGYSTVTLNVIRHHGTLSPVTLHWNIDSDPDGDLAFTSGNITFEIGQTSANITVEILPDEDPELDKA | | |
| | FSVSVLSVSSGSLGAHINATLTVLASDDPYGIFIFSEKNRPVKVEEATQNITLSIIRLKGLMGKVLV | | |
| | SYATLDDMEKPFYFPPNLARATGGRDYIPASGFALFGANGSEATIAISILDDDEPERSESVFIELLN | | |
| | STLVAKVQSRSIPNSPRLGPKVETIAQLIIIANDDAFGTLQLSAPIVRVAENHVGPIINVTRTGGAF | | |
| | ADVSVKFKAVPTTAIAGEDYSIASSDVVLLEGETSKAVPIYVINDIYPELEESFLVQLMNETTGGAR | | |
| | LGALTEAVIIIEASDDPYGLFGFQITKLIVEEPEFNSVKVNLPIIRNSGTLGNVTVQWVATINGQLA | | |
| | TGDLRVVSGNVTFAPGETIQTLLLEVLADDVPEIEEVIQVQLTDASCGGTIGLDRIANIIIPANDDP | | |
| | YGTVAFAQMVYRVQEPLERSSCANITVRRSGGHFGRLLLFYSTSDIDVVALAMEEGQDLLSYYESPI | | |
| | QGVPDPLWRTWNNVSAVGEPLYTCATLCLKEQACSAFSFFSASEGPQCFWMTSWISPAVNNSDFWTY | | |
| | RKNMTRVASLFSGQAVAGSDYEPVTRQWAIMQEGDEFANLTVSILPDDFPEMDESFLISLLEVHLMM | | |
| | ISASLKNQPTIGQPNISTVVIALNGDAFGVFVIYSISPNTSEDGLFVEVQEQPQTLVELMIHRTGGS | | |
| | LGQVAVEWRVVGGTATEGLDFIGAGEILTFAEGETKKTVILTILDDSEPEDDESIIVSLVYTEGGSR | | |
| | ILPSSDTVRVNILANDNVAGIVSFQTASRSVIGHEGEILQFHVIRTFPCRGNVTVNWKIIGQNLELN | | |
| | FANFSGQLFFFEGSLNTTLFVHLLDDNIPEEKEVYQVILYDVRTQGVPPAGIALLDAQGYAAVLTVE | | |
| | ASDEPHGVLNFALSSRFVLLQEANITIQLFINREFGSLCAINVTYTTVPGMLSLKNQTVGNLAEPEV | | |
| | DFVPIIGFLILEEGETAAAINITILEDDVPELEEYFLVNLTYVGLTMAASTSFPPRLDSEGLTAQVI | | |
| | IDANDGARGVIEWQQSRFEVNETHGSLTLVAQRSREPLGHVSLFVYAQNLEAQVGLDYIFTPMILHF | | |
| | ADGERYXNVNIMILDDDIPEGDEKFQLILTNPSPGLELGKNTIALIIVLANDDGPGVLSFNNSEHFF | | |
| | LREPTALYVQESVAVLYIVREPAQGLFGTVTVQFIVTEVNSSNESKDLTPSKGYIVLEEGVRFKALQ | | |
| | ISAILDTEPEMDEYFVCTLFNPTGGARLGVHVQTLITVLQNQAPLCLFSISAVENRATSIDIEEANR | | |
| | TVYLNVSRTNGIDLAVSVQWETVSETAFGMRGMDVVFSVFQSFLDESASGWCFFTLENLIYGIMLRK | | |
| | SSVTVYRWQGIFIPVEDLNIENPKTCEAFNIGFSPYFVITHEERNEEKPSLNSVFTFTSGFKLFLVQ | | |
| | TIIILESSQVRYFTSDSQDYLIIASQRDDSELTQVFRWNCGSFVLHQKLPVRGVLTVALFNXGGSVF | | |
| | LAISQANARLNSLLFRWSGSGFINFQEVPVSGTTEVEALSSANDIYLIFAXNVFLGDQNSIDIFIWE | | |
| | MGQSSFRYFQSVDFAAVNRIESFTPASGIAHILLIGQDMSALYCWNSERNQFSFVLEVPSAYDVASV | | |
| | TVXSLNSSKNLIALVGAHSHIYELAYISSHSDFIPSSGELIFEPGEREATIAVNILDDTVPEKEESF | | |
| | KVQLKNPKGGAEIGINDSVTITILSNDDAYGIVAFAQNSLYKQVEEMEQDSLVTLNVERLKGTYGRI | | |
| | TIAWEADGSISDIFPTSGVILFTEGQVLSTITLTILADNIPELSEVVIVTLTRITTEGVEDSYKGAT | | |
| | IDQDRSKSVITTLPNDSPFGLVGWRAASVFIRVAEPKENTTTLQLQIARDKGLLGDIAIHLRAQPNF | | |
| | LLHVDNQATENEDYVLQETIIIMKENIKEAHAEVSILPDDLPELEEGFIVTITEVNLVNSDFSTGQP | | |
| | SVRRPGMEIAEIMIEENDDPRGIFMFHVTRGAGEVITAYEVPPPLNVLQVPVVRLAGSFCAVNVYWK | | |
| | ASPDSAGLEDFKPSHGILEFADKQVTAMIEITIIDDAEFELTETFNISLISVAGGGRLGDDVVVTVV | | |
| | IPQNDSFFGVFCFEEKTVS | | |
| | SEQ ID NO: 163 | 5102 bp | |
| NOV39c, | CAGGGAAAACGGAACCTATCGAATGGTCATGGTGACTTTTGAGGTAGAGGGTGGCCCAAATCCCCCT | | |
| CG150799-03 | | | |
| DNA Sequence | GATGAAGATTTGAGTCCAGTTAAACGAAATATCACCTTTCCCCCTGGCAGAGCAACAGTAATTTATA | | |
| | ACTTGACAGTACTCGATGACGAGGTACCAGAAAATGATGAAATATTTTTAATTCAACTGAAAAGTGT | | |
| | AGAAGGAGGAGCTGAGATTAACACCTCTAGGAATTCCATTGACATCATCATTAACAAAAATGATAGT | | |
| | CCCGTGAGATTCCTTCAGAGTATTTATTTGGTTCCTCAGGAACACCACATACTCATAATTCCAGTAG | | |
| | TTCGTGGAAAGGACAACAATGGAAATCTGATTGGATCTGATGAATATGAGGTTTCAATCAGTTATGC | | |
| | TGTCACAACTGGGAATTCCACAGCACATGCCCAGCAAAATCTCCACTTCATTGATCTTCAGCCAAAC | | |
| | ACAACTGTTGTTTTTCCACCTTTTATTCATGAATCTCACTTGAAATTTCAAATAGTTGATGACACCA | | |
| | CACCGGAGATTGCTGAATCGTTTCACATTATGTTACTAAAACATACCTTACAGGGAGATGCTGTGCT | | |
| | AATAAGCCCTTCTGTTGTACAACTCACCATTAAGCCAAATGATAAACCTTATGGAGTCCTTTCATTC | | |
| | AACAGTGTTTTGTTTGAAACGACAGTTATAATTGATGAAGATACAATATCAAGATATGAAGAAATGA | | |
| | CAGTGGTTAGAAATGGAGGAACCCATGGGAATGTCTCTCCGAATTGGGTGTTGACACGCAACAGCAC | | |
| | TGATCCCTCACCACTAACAGCAGATATCAGACCGAGCTCTGGAGTTCTCCATTTTGCACAACGGCAG | | |
| | ATCTTGGCAACAATTCCTCTTACTGTGGTTGATGATGATCTTCCAGAAGAGGCAGAAGCTTATCTAC | | |
| | TTCAAATTCTGCCTCATACAATACGAGGAGGTGCAGAAGTGAGCGACCCACCGGAGGATAGTGATGA | | |
| | TGTCTATGGCCTAATAACATTTTTTCCTATGGAAAACCAGAAGATTCAAAGCAGCCCAGGTGAACGA | | |
| | TACTTATCCTTGAGTTTTACAAGACTAGGAGGGACTAAAGGAGATCTCAGCTTGCTTTATTCTGTAC | | |
| | TTTACATTCCTGCTGGAGCTGTGGACCCCTTGCAAGCAAAAGAAGCCATCTTAAATATATCAAGGAG | | |
| | AAATGACCTCATTTTTCCAGAGCAAAAAACTCAAGTCACTACAAAATTACCAATAAGAAATGATGCA | | |
| | TTCTTTCAAAATGGAGCTCACTTTCTAGTACAGTTGGAAACTGTGCAGTTGTTAAACATAATTCCTC | | |
| | TAATCCCACCCATAAGCCCTAGATTTGGGGAAATCTGCAATATTTCTTTACTCGTTACTCCAGCCAT | | |
| | TGCAAATGGACAAATTGGCTTTCTCAGCAATCTTCCAATTATTTTGCATGAACCAGAAGATTTTGCT | | |
| | GCTGAAGTGGTATACATTCCCTTACATCGGGATGGAACTGATGGCCAGGCTACTGTCTACTGGAGTT | | |
| | TGAAGCCCTCTGGCTTTAATTCAAAAGCAGTGACCCCGGATGATATAGGCCCCTTTAATGGCTCTGT | | |
| | TTTCTTTTTATCTGGGCAAAGTGACACAACAATCAACATTACTATCAAAGGTGATGACATACCCGAA | | |
| | ATGAATGAAACTGTAACACTTTCTCTAGACAGGGTTAACGTGGAAAACCAACTGCTGAAATCTGGAT | | |
| | ATACTAGCCGTGACCTAATTATTTTGGAAAATGATCACCCTCGGGGAGTTTTTGAATTTTCTCCTGC | | |
| | TTCCAGAGGACCCTATGTTATAAAAGAACGAGAATCTGTAGAGCTCCACATCATCCGATCAAGGGGG | | |
| | TCCCTTGTTAAGCAGTTTCTACACTACCGAGTAGAGCCAAGAGATAGCAATGAATTCTATGGAAACA | | |
| | CGGGAGTACTAGAATTTAAACCTGGAGAAAGGGAGATAGTGATCACCTTGCTAGCAAGATTCGATGG | | |
| | GATACCAGAGTTGGATGAACACTACTGGGTGGTCCTCAGCAGCCACGGAGAACGGGAAAGCAAGTTG | | |
| | GGAAGTGCCACCATTGTCAATATAACGATTCTGAAAAATGATCATCCTCATGGCATTATAGAATTTG | | |
| | TTTCTGATGGTCTAATTGTGATGATAAATGAAAGCAAAGGACATGCTATCTATAGTGCTGTTTATGA | | |
| | TGTAGTAAGAAATCGAGGCAACTTTGGTGATGTTAGTGTATCATGCGTGGTTAGTCCAGACTTTACA | | |
| | CAAGATGTATTTCCTGTACAAGGGACTGTTGTCTTTGCAGATCAGCAATTTTCAAAAAATATCACCA | | |
| | TTTACTCCCTTCCAGATGAGATTCCAGAAGAAATGGAAGAATTTACCGTTATCCTACTGAATGGCAC | | |
| | TGGAGGAGCTAAAGTGGGAAATAGAACAACTGCAACTCTCAGGATTAGAAGAAATGATGACCCCATT | | |
| | TATTTTGCACAACCTCGTGTAGTGAGGGTTCACGAAGGTGAGACTGCCAACTTTACAGTTCTCAGAA | | |
| | ATCGATCTGTTGATGTGACTTGCATGGTCCAGTATGCTACCAACGATGGGAAGGCTACTGCAAGAGA | | |
| | GAGAGATTTCATTCCTGTTGAAAAAGGAGAAACGCTCATTTTTGAGGTTGGAAGTAGACAGCAGAGC | | |
| | ATATCCATATTTGTTAATGAAGATGGTATCCCGGAAACAGATGAGCCCTTTTATATAATCCTCTTGA | | |
| | ATTCAACAGGTGATACAGTAGTATATCAATATGGAGTAGCTACAGTAATAATTGAAGCTAATGATGA | | |
| | CCCAAATGGCATTTTTTCTCTGGAGCCCATAGACAAAGCAGTGCAAGAAGGAAAGACTAATGCATTT | | |
| | TGGATTTTGAGGCACCGAGGATACTTTGGTAGTGTTTCTGTATCTTGGCAGCTCTTTCAGAATGATT | | |
| | CTGCTTTGCAGCCTGGGCAGGAGTTCTATGAAACTTCAGGAACTGTTAACTTCATGGATGGAGAAGA | | |
| | AGCAAAACCAATCATTCTCCATGCTTTTCCAGATAAAATTCCTGAATTCAATGAATTTTATTTCCTA | | |
| | AAACTTGTAAACATTTCAGGTCCTGGGGGCCAGCTAGCAGAAACCAACCTCCAGGTGACAGTAATGG | | |
| | TTCCATTCAATGATGATCCCTTTGGAGTTTTTATCTTGGATCCAGAGTGTTTAGAGAGAGAAGTGGC | | |
| | AGAAGATGTCCTGTCTGAAGATGATATGTCTTATATTACCAACTTCACCATTTTGAGGCAGCAGGGT | | |
| | GTGTTTGGTGATGTACAACTGGGCTGGGAAATACTCTCCAGTGAGTTCCCTGCTGGTTTGCCACCAA | | |
| | TGATAGATTTTTTACTGGTTGGAATTTTCCCCACCACCGTGCATTTACAACAGCACATGCGGCGTCA | | |
| | CCACAGTGGAACGGATGCTTTGTACTTTACCGGACTAGAGCGTGCATTTGGGACTGTTAATCCAAAA | | |
| | TACCATCCCTCCAGGAATAATACAATTGCCAACTTTACATTCTCAGCTTGGGTAATGCCCAATGCCA | | |
| | ATACGAATGGATTCATTATAGCGAAGGATGACGGTAATGGAAGCATCTACTACGGGGTAAAAATACA | | |
| | AACAAACGAATCCCATGTGACACTTTCCCTTCATTATAAAACCTTCGGTTCCAATGCTACATACATT | | |
| | GCCAAGACAACAGTCATGAAATATTTAGAAGAAAGTGTTTGGCTTCATCTACTAATTATCCTGGAGG | | |
| | ATGGTATAATCGAATTCTACCTGGATGGAAATGCAATGCCCAGGGGAATCAAGAGTCTGAAAGGAGA | | |
| | AGCCATTACTGACGGTCCTGGGATACTGAGAATTGGAGCAGCGATAAATGGCAATGACAGATTTACA | | |
| | GGTCTGATGCAGGATGTGAGGTCCTATGAGCGGAAACTGACGCTTGAAGAAATTTATGAACTTCATG | | |
| | CCATGCCCGCAAAAAGTGATTTACACCCAATTTCTGGATATCTGGAGTTCAGACAGGGAGAAACTAA | | |
| | CAAATCATTCATTATTTCTGCAAGAGATGACAATGACGAGGAAGGAGAAGAATTATTCATTCTTAAA | | |
| | CTAGTTTCTGTATATGGAGGAGCTCGTATTTCGGAAGAAAATACTACTGCAAGATTAACAATACAAA | | |
| | AAAGTGACAATGCAAATGGCTTGTTTGGTTTCACAGGAGCTTGTATACCAGAGATTGCAGAGGAGGG | | |
| | ATCAACCATTTCTTGTGTGGTTGAGAGAACCAGAGGAGCTCTGGATTATGTGCATGTTTTTTACACC | | |
| | ATTTCACAGATTGAAACTGATGGCATTAATTACCTTGTTGATGACTTTGCTAATGCCAGTGGAACTA | | |
| | TTACATTCCTTCCTTGGCAGAGATCAGAGCTTTTGATTGAAGTGTCGCTTCCCATTATTATTTACAA | | |
| | CTGTAACTGATACATTAGAATTTGCTTCAAACATGTCTGCTGTAAACCTTTATCAGGTTCTGCAATA | | |
| | TATATGTTCTTGATGATGATATTCCTGAACTTAATGAGTATTTCCGTGTGACATTGGTTTCTGCAAT | | |
| | TCCTGGAGATGGGAAGCTAGGCTCAACTCCTACCAGTGGTGCAAGCATAGATCCTGAAAAGGAAACC | | |
| | ACTGATATCACCATCAAAGCTAGTGATCATCCATATGGCTTGCTGCAGTTCTCCACAGGGCTGCCTC | | |
| | CTCAGCCTAAGGACGCAATGACCCTGCCTGCAAGCAGCGTTCCACATATCACTGTGGAGGAGGAAGA | | |
| | TGGAGAAATCAGGTTATTGGTCATCCGTGCACAGGGACTTCTGGGAAGGGTGACTGCGGAATTTAGA | | |
| | ACAGTGTCCTTGACAGCATTCAGTCCTGAGGATTACCAGAATGTTGCTGGCACATTAGAATTTCAAC | | |
| | CAGGACAAAGATATAAATACATTTTCATAAACATCACTGATAATTCTATTCCTGAACTGGAAAAATC | | |
| | TTTTAAAGTTGAGTTGTTAAACTTGGAAGGAGGAGCTCTGCTAGATCTATCTACAGATATAACGCTG | | |
| | TAAAATCTGGTCCTTTTGGATGATCTATAATGAGTTGATTATTAATAAAAGAAGTCAACAATACCTT | | |
| | AAAAAAAAAA | | |
| | ORF Start: ATG at 23 | | ORF Stop: TGA at 4430 |
| | SEQ ID NO: 164 | 1469 aa | MW at 162809.6 kD |
| NOV39c, | MVMVTFEVEGGPNPPDEDLSPVKGNITFPPGRATVIYNLTVLDDEVPENDEIFLIQLKSVEGGAEIN | | |
| CG150799-03 | | | |
| Protein Sequence | TSRNSIEIIIKKNDSPVRFLQSIYLVPEEDHILIIPVVRGKDNNGNLIGSDEYEVSISYAVTTGNST | | |
| | AHAQQNLDFIDLQPNTTVVFPPFIHESHLKFQIVDDTTPEIAESFHIMLLKDTLQGDAVLISPSVVQ | | |
| | VTIKPNDKPYGVLSFNSVLFERTVIIDEDRISRYEEITVVRNGGTHGNVSANWVLTRNSTDPSPVTA | | |
| | DIRPSSGVLHFAQGQMLATIPLTVVDDDLPEEAEAYLLQILPHTIRGGAEVSEPAEDSDDVYGLITF | | |
| | FPMENQKIESSPGERYLSLSFTRLGGTKGDVRLLYSVLYIPAGAVDPLQAKEGILNISRRNDLIFPE | | |
| | QKTQVTTKLPXRNDAFFQNGAHFLVQLETVELLNIIPLIPPISFRFGEICNISLLVTPAIANGEIGF | | |
| | LSNLPIILHEPEDFAAEVVYIPLHRDGTDGQATVYWSLKPSGFNSKAVTPDDIGPFNGSVLFLSGQS | | |
| | DTTINITIKGDDIPEMNETVTLSLDRVNVENQVLKSGYTSRDLIILENDDPGGVFEFSPASRGPYVI | | |
| | KEGESVELHIIRSRGSLVKQFLHYRVEPRDSNEFYGNTGVLEFKPGEREIVTTLLARLDGIPELDEH | | |
| | YWVVLSSHGERESKLGSATIVNITILKNDDPHGIIEFVSDGLIVMINESKGDAIYSAVYDVVRNRGN | | |
| | FGDVSVSWVVSPDFTQDVFPVQGTVVFGDQEFSKNITIYSLPDEIPEEMEEFTVILLNGTGGAKVGN | | |
| | RTTATLRIRRNDDPIYFAEPRVVRVQEGETANFTVLRNGSVDVTCMVQYATKDGKATARERDFIPVE | | |
| | KGETLIFEVGSRQQSISIFVNEDGIPETDEPFYIILLNSTGRTVVYQYGVATVIIEANDDPNGIFSL | | |
| | EPIDKAVEEGKTNAFWILRHRGYFGSVSVSWQLFQNDSALQPGQEFYETSGTVNFMDGEEAXPIILH | | |
| | AFPDKIPEFNEFYFLKLVNTSGPGCQLAETNLQVTVMVPFNDDPFGVFILDPECLEREVAEDVLSED | | |
| | DMSYITNFTILRQQGVFGDVQLGWEILSSEFPAGLPPMIDFLLVGIFPTTVHLQQHMRRHHSGTDAL | | |
| | YFTGLEGAFGTVNPKYHPSRNNTIANFTFSAWVMPNANTNGFIIAKDDGNGSIYYGVKIQTNESHVT | | |
| | LSLHYKTLGSNATYIAKTTVMXYLEESVWLHLLIILEDGIIEFYLDGNAMPRGIKSLKGEAITDGPG | | |
| | ILRIGAGINGNDRFTGLMQDVRSYERKLTLEEIYELHAMPAKSDLHPISGYLEFRQGETNKSFIISA | | |
| | RDDNDEEGEELFILKLVSVYGGARISEENTTARLTIQKSDNANGLFGFTGACIPEIAEEGSTISCVV | | |
| | ERTRGALDYVHVFYTISQIETDGINYLVDDFANASGTITFLPWQRSELLIEVSLPIIIYNCN | | |
| | SEQ ID NO: 165 | 8350 bp | |
| NOV39d, | CAGGGAAAAGGGAACCTATGGAATGGTCATGGTGACTTTTGAGGTAGAGGGTGGCCCAAATCCCCCT | | |
| CG150799-01 | | | |
| DNA Sequence | GATGAAGATTTGAGTCCAGTTAAAGGAAATATCACCTTTCCCCCTGGCAGACCAACAGTAATTTATA | | |
| | ACTTGACAGTACTCGATGACGAGGTACCAGAAAATGATGAAATATTTTTAATTCAACTGAAAAGTGT | | |
| | AGAAGGACGAGCTGAGATTAACACCTCTAGGAATTCCATTGAGATCATCATTAAGAAAAATGATAGT | | |
| | CCCGTGAGATTCCTTCAGAGTATTTATTTGGTTCCTGAGGAAGACCACATACTCATAATTCCAGTAG | | |
| | TTCGTGGAAACGACAACAATCGAAATCTGATTGGATCTGATGAATATGAGGTTTCAATCAGTTATGC | | |
| | TCTCACAACTGGGAATTCCACAGCACATGCCCAGCAAAATCTGGACTTCATTGATCTTCAGCCAAAC | | |
| | ACAACTGTTGTTTTTCCACCTTTTATTCATGAATCTCACTTGAAATTTCAAATAGTTGATGACACCA | | |
| | CACCGGAGATTGCTGAATCGTTTCACATTATGTTACTAAAAGATACCTTACAGGGAGATGCTGTGCT | | |
| | AATAAGCCCTTCTGTTGTACAAGTCACCATTAAGCCAAATGATAAACCTTATGGAGTCCTTTCATTC | | |
| | AACAGTGTTTTGTTTGAAACGACAGTTATAATTGATGAAGATAGAATATCAAGATATGAAGAAATCA | | |
| | CAGTGGTTAGAAATGGAGGAACCCATGGGAATGTCTCTGCGAATTGGGTGTTGACACGGAACAGCAC | | |
| | TGATCCCTCACCAGTAACAGCAGATATCAGACCGAGCTCTCGAGTTCTCCATTTTGCACAAGGGCAG | | |
| | ATGTTGGCAACAATTCCTCTTACTGTGGTTGATGATGATCTTCCAGAAGAGGCAGAAGCTTATCTAC | | |
| | TTCAAATTCTGCCTCATACAATACGAGGAGGTGCAGAAGTGAGCGAGCCAGCGGACGATAGTGATGA | | |
| | TGTCTATGGCCTAATAACATTTTTTCCTATGGAAAACCAGAAGATTGAAAGCAGCCCAGGTGAACGA | | |
| | TACTTATCCTTGAGTTTTACAAGACTAGGAGGGACTAAAGGAGATGTGAGGTTGCTTTATTCTGTAC | | |
| | TTTACATTCCTGCTGCAGCTGTGGACCCCTTGCAAGCAAAAGAAGGCATCTTAAATATATCAAGGAG | | |
| | AAATGACCTCATTTTTCCAGAGCAAAAAACTCAAGTCACTACAAAATTACCAATAAGAAATGATGCA | | |
| | TTCTTTCAAAATGGAGCTCACTTTCTAGTACAGTTGGAAACTCTGGAGTTGTTAAACATAATTCCTC | | |
| | TAATCCCACCCATAAGCCCTAGATTTGGGGAAATCTGCAATATTTCTTTACTGGTTACTCCAGCCAT | | |
| | TGCAAATGGAGAAATTCGCTTTCTCAGCAATCTTCCAATTATTTTGCATGAACCAGAAGATTTTGCT | | |
| | GCTGAAGTGGTATACATTCCCTTACATCGGGATGGAACTGATGGCCAGGCTACTGTCTACTGGAGTT | | |
| | TGAAGCCCTCTGGCTTTAATTCAAAAGCAGTGACCCCGGATGATATAGGCCCCTTTAATGGCTCTGT | | |
| | TTTGTTTTTATCTGGGCAAAGTGACACAACAATCAACATTACTATCAAAGGTGATGACATACCGGAA | | |
| | ATGAATGAAACTGTAACACTTTCTCTAGACAGGGTTAACGTGGAAAACCAAGTGCTGAAATCTGGAT | | |
| | ATACTAGCCGTGACCTAATTATTTTGGAAAATGATGACCCTGGGGGAGTTTTTGAATTTTCTCCTGC | | |
| | TTCCAGAGGACCCTATGTTATAAAACAAGGAGAATCTGTAGAGCTCCACATCATCCGATCAAGGGGG | | |
| | TCCCTTGTTAAGCAGTTTCTACACTACCGAGTAGAGCCAAGAGATAGCAATGAATTCTATGGAAACA | | |
| | CGGGAGTACTAGAATTTAAACCTGGAGAAACCGAGATAGTGATCACCTTGCTAGCAAGATTGGATGG | | |
| | GATACCAGAGTTGGATGAACACTACTGGGTGGTCCTCAGCAGCCACGGAGAACGGGAAAGCAAGTTG | | |
| | GGAAGTGCCACCATTGTCAATATAACGATTCTGAAAAATGATGATCCTCATGGCATTATAGAATTTC | | |
| | TTTCTGATGGTCTAATTGTGATGATAAATGAAAGCAAAGGAGATGCTATCTATAGTGCTGTTTATGA | | |
| | TGTAGTAAGAAATCGAGGCAACTTTGGTCATGTTAGTGTATCATGGGTGGTTAGTCCAGACTTTACA | | |
| | CAAGATGTATTTCCTGTACAAGGGACTGTTGTCTTTGGAGATCAGGAATTTTCAAAAAATATCACCA | | |
| | TTTACTCCCTTCCAGATGAGATTCCAGAAGAAATGGAAGAATTTACCGTTATCCTACTGAATGGCAC | | |
| | TGGAGGAGCTAAAGTGGGAAATAGAACAACTGCAACTCTGAGGATTAGAAGAAATGATGACCCCATT | | |
| | TATTTTGCAGAACCTCGTGTAGTGAGGGTTCAGGAAGGTGAGACTGCCAACTTTACAGTTCTCAGAA | | |
| | ATGGATCTGTTGATGTGACTTGCATGGTCCAGTATGCTACCAAGGATGGGAAGGCTACTGCAAGAGA | | |
| | GAGAGATTTCATTCCTGTTGAAAAAGGAGAAACGCTCATTTTTGAGGTTGGAAGTAGACAGCAGAGC | | |
| | ATATCCATATTTGTTAATGAAGATGGTATCCCGGAAACACATGAGCCCTTTTATATAATCCTCTTGA | | |
| | ATTCAACAGGTGATACAGTAGTATATCAATATGGAGTAGCTACAGTAATAATTGAAGCTAATGATGA | | |
| | CCCAAATGGCATTTTTTCTCTGGAGCCCATAGACAAAGCAGTGGAAGAAGGAAAGACTAATGCATTT | | |
| | TGGATTTTGAGGCACCGAGGATACTTTGGTAGTGTTTCTGTATCTTGGCAGCTCTTTCAGAATGATT | | |
| | CTGCTTTGCAGCCTGGGCAGGAGTTCTATGAAACTTCAGGAACTGTTAACTTCATGGATGGAGAAGA | | |
| | AGCAAAACCAATCATTCTCCATCCTTTTCCAGATAAAATTCCTGAATTCAATGAATTTTATTTCCTA | | |
| | AAACTTGTAAACATPTCAGGTCCTGGGGGCCAGCTAGCAGAAACCAACCTCCAGGTGACAGTAATGG | | |
| | TTCCATTCAATGATGATCCCTTTGGAGTTTTTATCTTGGATCCAGAGTGTTTAGAGAGAGAAGTGGC | | |
| | AGAAGATGTCCTGTCTGAAGATGATATGTCTTATATTACCAACTTCACCATTTTGAGGCAGCAGGGT | | |
| | GTGTTTGGTGATGTACAACTGGGCTGGGAAATACTGTCCAGTGAGTTCCCTGCTGGTTTGCCACCAA | | |
| | TGATAGATTTTTTACTGGTTGCAATTTTCCCCACCACCGTGCATTTACAACAGCACATGCGGCGTCA | | |
| | CCACAGTGGAACGGATGCTTTGTACTTTACCGGACTAGAGGGTGCATTTGGGACTGTTAATCCAAAA | | |
| | TACCATCCCTCCAGGAATAATACAATTGCCAACTTTACATTCTCAGCTTGGGTAATGCCCAATGCCA | | |
| | ATACGAATGGATTCATTATAGCGAACGATGACGGTAATGGAAGCATCTACTACGGGGTAAAAATACA | | |
| | AACAAACGAATCCCATGTGACACTTTCCCTTCATTATAAAACCTTGGGTTCCAATGCTACATACATT | | |
| | GCCAAGACAACAGTCATGAAATATTTAGAAGAAAGTGTTTGGCTTCATCTACTAATTATCCTGGAGG | | |
| | ATGGTATAATCGAATTCTACCTGGATGGAAATGCAATGCCCAGGGGAATCAAGAGTCTGAAAGGAGA | | |
| | AGCCATTACTGACGGTCCTGCGATACTGAGAATTGGAGCAGGGATAAATGGCAATGACAGATTTACA | | |
| | GGTCTGATGCAGGATGTGAGGTCCTATGAGCGGAAACTGACGCTTGAAGAAATTTATGAACTTCATG | | |
| | CCATGCCCGCAAAAAGTGATTTACACCCAATTTCTGGATATCTGGAGTTCAGACAGGGAGAAACTAA | | |
| | CAAATCATTCATTATTTCTGCAAGAGATGACAATGACGAGGAAGGAGAAGAATTATTCATTCTTAAA | | |
| | CTAGTTTCTGTATATGGAGGAGCTCGTATTTCGGAAGAAAATACTACTGCAAGATTAACAATACAAA | | |
| | AAAGTGACAATGCAAATGGCTTGTTTGGTTTCACAGGAGCTTGTATACCAGAGATTGCAGAGGAGGG | | |
| | ATCAACCATTTCTTGTGTCGTTGAGAGAACCAGAGGAGCTCTGGATTATGTGCATGTTTTTTACACC | | |
| | ATTTCACAGATTGAAACTGATGGCATTAATTACCTTGTTGATGACTTTGCTAATGCCAGTGGAACTA | | |
| | TTACATTCCTTCCTTGGCAGAGATCAGAGGTTCTGAATATATATGTTCTTGATGATGATATTCCTGA | | |
| | ACTTAATGAGTATTTCCGTGTGACATTGGTTTCTGCAATTCCTGGACATGGGAAGCTAGGCTCAACT | | |
| | CCTACCAGTGGTGCAAGCATAGATCCTGAAAAGGAAACGACTGATATCACCATCAAAGCTAGTGATC | | |
| | ATCCATATGGCTTGCTGCAGTTCTCCACAGGGCTGCCTCCTCAGCCTAAGGACGCAATCACCCTGCC | | |
| | TGCAAGCAGCGTTCCACATATCACTGTGGAGGAGGAAGATGGAGAAATCAGGTTATTGGTCATCCGT | | |
| | GCACAGGGACTTCTGGGAAGGGTGACTGCGGAATTTAGAACAGTGTCCTTGACAGCATTCAGTCCTG | | |
| | AGGATTACCAGAATGTTGCTGGCACATTAGAATTTCAACCAGGAGAAAGATATAAATACATTTTCAT | | |
| | AAACATCACTGATAATTCTATTCCTGAACTGGAAAAATCTTTTAAAGTTGAGTTGTTAAACTTGGAA | | |
| | GGACGAGTAGCTGAACTCTTTAGGGTTCATGGAAGTGGTAGTGCCAGTCTAGGAGTGGCTTCCCAAA | | |
| | TTCTAGTGACAATTGCAGCCTCTGACCACGCTCATGGCGTATTTGAATTTAGCCCTGAGTCACTCTT | | |
| | TGTCAGTGGAACTGAACCAGAAGATGGGTATAGCACTGTTACATTAAATGTTATAAGACATCATGGA | | |
| | ACTCTGTCTCCAGTGACTTTGCATTGGAACATAGACTCTGATCCTGATGGTGATCTCGCCTTCACCT | | |
| | CTGGCAACATCACATTTGAGATTGGGCAGACGAGCGCCAATATCACTGTGGAGATATTGCCTGACGA | | |
| | AGACCCAGAACTGGATAAGGCATTCTCTGTGTCAGTCCTCAGTGTTTCCAGTGGTTCTTTGGGAGCT | | |
| | CATATTAATGCCACGTTAACAGTTTTGGCTAGTGATGATCCATATGGGATATTCATTTTTTCTGAGA | | |
| | AAAACAGACCTGTTAAAGTTGAGGAAGCAACCCAGAACATCACACTATCAATAATAAGGTTGAAAGG | | |
| | CCTCATGGGAAAAGTCCTTGTCTCATATCCAACACTAGATGATATGGAAAAACCACCTTATTTTCCA | | |
| | CCTAATTTAGCGAGAGCAACTCAAGGAAGAGACTATATACCAGCTTCTGGATTTGCTCTTTTTGGAG | | |
| | CTAATCAGAGTGAGGCAACAATAGCTATTTCAATTTTGGATGATGATGAGCCAGAAAGGTCCGAATC | | |
| | TGTCTTTATCGAACTACTCAACTCTACTTTAGTAGCGAAAGTACAGAGTCGTTCAATTCCAAATTCT | | |
| | CCACGTCTTGGGCCTAAGGTAGAAACTATTGCGCAACTAATTATCATTGCCAATGATGATGCATTTG | | |
| | GAACTCTTCAGCTCTCAGCACCAATTGTCCGAGTGGCAGAAAATCATGTTGGACCCATGATCAATGT | | |
| | GACTAGAACAGGAGGAGCATTTGCAGATGTCTCTGTGAAGTTTAAAGCTGTGCCAATAACTGCAATA | | |
| | GCTGGTGAAGATTATAGTATAGCTTCATCAGATGTGGTCTTGCTAGAAGGGGAAACCAGTAAAGCCG | | |
| | TGCCAATATATGTCATTAATGATATCTATCCTGAACTGGAAGAATCTTTTCTTGTGCAACTGATGAA | | |
| | TGAAACAACAGGAGGAGCCAGACTAGGGGCTTTAACACAGGCAGTCATTATTATTGAGGCCTCTGAT | | |
| | GACCCCTATGGATTATTTGGTTTTCAGATTACTAAACTTATTGTAGAGGAACCTGAGTTTAACTCAG | | |
| | TGAAGGTAAACCTGCCAATAATTCGAAATTCTGGGACACTCGGCAATGTTACTGTTCAGTGGGTTGC | | |
| | CACCATTAATGGACAGCTTGCTACTGGCGACCTGCGAGTTGTCTCAGGTAATGTGACCTTTGCCCCT | | |
| | GGGGAAACCATTCAAACCTTGTTGTTAGAGGTCCTGGCTGACGACGTTCCGGAGATTGAAGAGGTTA | | |
| | TCCAAGTGCAACTAACTGATGCCTCTGGTGCAGGTACTATTGGGTTAGATCGAATTGCAAATATTAT | | |
| | TATTCCTGCCAATGATGATCCTTATGGTACAGTAGCCTTTGCTCAGATGGTTTATCGTGTTCAAGAG | | |
| | CCTCTGGAAAGAAGTTCCTGTGCTAATATAACTGTCAGGCGAAGCGGAGGCCACTTTGGTCGGCTGT | | |
| | TGTTGTTCTACAGTACTTCCGACATTGATGTAGTGGCTCTGGCAATGGAGGAAGGTCAAGATTTACT | | |
| | GTCCTACTATGAATCTCCAATTCAAGGGCTGCCTGACCCACTTTGGAGAACTTGGATGAATGTCTCT | | |
| | GCCGTGGGGGAGCCCCTGTATACCTGTGCCACTTTGTGCCTTAAGGAACAAGCTTGCTCAGCGTTTT | | |
| | CATTTTTCAGTGCTTCTGAGGGTCCCCAGTGTTTCTGGATGACATCATGGATCAGCCCAGCTGTCAA | | |
| | CAATTCAGACTTCTGGACCTACAGGAAAAACATGACCAGGGTAGCATCTCTTTTTAGTGGTCAGGCT | | |
| | GTGGCTGGGAGTGACTATGAGCCTGTCACAAGGCAATGGGCCATAATGCAGGAAGGTGATGAATTCG | | |
| | CAAATCTCACAGTGTCTATTCTTCCTGATGATTTCCCAGAGATGGATGAGAGTTTTCTAATTTCTCT | | |
| | CCTTGAAGTTCACCTCATGAACATTTCAGCCAGTTTGAAAAATCAGCCAACCATAGGACAGCCAAAT | | |
| | ATTTCTACAGTTGTCATAGCACTAAATGGTGATGCCTTTGGAGTGTTTGTGATCTACAATATTAGTC | | |
| | CCAATACTTCCGAAGATGGCTTATTTGTTGAAGTTCAGGAGCAGCCCCAAACCTTGGTGCAGCTGAT | | |
| | GATACACAGGACAGGGGGCAGCTTAGGTCAAGTGGCAGTCGAATGGCGTGTTGTTGGTGGAACAGCT | | |
| | ACTGAAGGTTTAGATTTTATAGGTGCTGGAGAGATTCTGACCTTTGCTGAAGGTGAAACCAAAAAGA | | |
| | CAGTCATTTTAACCATCTTGGATGACTCTGAACCAGAGGATGACGAAAGTATCATAGTTAGTTTGGT | | |
| | CTACACTGAAGGTGGAAGTAGAATTTTGCCAAGCTCCCACACTGTTAGAGTGAACATTTTGGCCAAT | | |
| | GACAATGTGGCAGGAATTGTTAGCTTTCAGACACCTTCCAGATCTGTCATAGCTCATGAAGGAGAAA | | |
| | TTTTACAATTCCATGTGATAAGAACTTTCCCTGGTCGAGGAAATGTTACTGTTAACTGGAAAATTAT | | |
| | TGGGCAAAATCTAGAACTCAATTTTGCTAACTTTAGCGGACAACTTTTCTTTCCTGAGGGGTCGTTC | | |
| | AATACAACATTGTTTGTGCATTTGTTGGATGACAACATTCCTGAGGAGAAAGAAGTATACCAAGTCA | | |
| | TTCTGTATGATGTCAGGACACAAGGAGTTCCACCAGCCCGAATCGCCCTGCTTGATGCTCAAGGATA | | |
| | TGCAGCTGTCCTCACAGTAGAAGCCAGTGATGAACCACATGGAGTTTTAAATTTTGCTCTTTCATCA | | |
| | AGATTTGTGTTACTACAAGAGGCTAACATAACAATTCAGCTTTTCATCAACAGAGAATTTGGATCTC | | |
| | TAGGAGCTATCAATGTCACATATACCACGGTTCCTGGAATGCTGAGTCTGAAGAACCAAACAGTAGG | | |
| | AAACCTAGCAGAGCCAGAAGTTGATTTTGTCCCTATCATTGGCTTTCTGATTTTAGAAGAAGGGGAA | | |
| | ACAGCAGCAGCCATCAACATTACCATTCTTGAGGATGATGTACCAGAGCTAGAAGAATATTTCCTGG | | |
| | TGAATTTAACTTACGTTGGACTTACCATGGCTGCTTCAACTTCATTTCCTCCCAGACTAGGTATGAG | | |
| | GGGTTTCTTGTTTGTTTCTTTTTGCTCACTTCAAATGAAATGAAGAAACTTCATTTTTGAATCAGAA | | |
| | GTGATCATTGTGCTGTTTTGTTAATCTTAGCTATGTGTTAAA | | |
| | ORF Start: ATG at 23 | | ORF Stop: TGA at 8282 |
| | SEQ ID NO: 166 | 2753 aa | MW at 301743.8 kD |

| | |
|---|---|
| NOV39d, | MVMVTFEVEGGPNPPDEDLSPVKGNITFPPGRATVIYNLTVLDDEVFENDEIELTQLKSVEGGAEIN |
| CG150799-01 | |
| Protein Sequence | TSRNSIEIIIKKNDSPVRFLQSIYLVPEEDHILIIPVVRGKDNNGNLIGSDEYEVSISYAVTTGNST |
| | AHAQQNLDFIDLQPNTTVVFPPFIHESHLKFQIVDDTTPEIAESFHIMLLKDTLQGDAVLISPSVVQ |
| | VTIKPNDKPYGVLSFNSVLFERTVIIDEDRISRYEEITVVRNGGTHGNVSANWVLTRNSTDPSPVTA |
| | DIRPSSGVLHFAQGQMLATIPLTVVDDDLPEEAEAYLLQILPHTIRGGAEVSEPAEDSDDVYGLITF |
| | FPMENQKIESSPGERYLSLSFTRLGGTKGDVRLLYSVLYIPAGAVDPLQAKEGILNISRRNDLIFPE |
| | QKTQVTTKLPIRNDAFFQNGAHFLVQLETVELLNIIPLIPPISPRFGEICNISLLVTPAIANGEIGF |
| | LSNLPIILHEPEDFAAEVVYIPLHRDGTDGQATVYWSLKPSGFNSKAVTPDDIGPFMGSVLFLSGQS |
| | DTTINITIKGDDIPFMNETVTLSLDRVNVENQVLKSGYTSRDLIILENDDPGGVFEFSPASRGPYVI |
| | KEGESVELHIIRSRGSLVKQFLHYRVEPRDSNEFYGNTGVLEFKPGEREIVITLLARLDGIPELDEH |
| | YWVVLSSHGERESKLGSATIVNITILKNDDFHGIIEFVSDGLIVMINESKGDAIYSAVYDVVRNRGN |
| | FGDVSVSWVVSPDFTGDVFPVGCTVVFGDGEFSKNITIYSLPDEIPEEMEEFTVILLNGTGGAKVGN |
| | RTTATLRIRRNDDPIYFAEPRVVRVQEGETANFTVLRNGSVDVTCMVQYATKDGKATARERDFIPVE |
| | KGETLIFEVGSRQQSISIFVNEDGIPETDEPFYTILLNSTGDTVVYQYGVATVIIEANDDPNGIFSL |
| | EPIDKAVEEGKTNAFWILRHRGYFGSVSVSWQLFQNDSALQPGQEFYETSGTVNFMDGEEAKPIILH |
| | AFPDKIPEFNEFYFLKLVNISGPGGQLAETNLQVTVMVPFNDDPFGVFILDPECLEREVAEDVLSED |
| | DMSYITNFTILRQQGVFGDVQLGWEILSSEFPAGLPFMIDFLLVGIFFTTVHLQQHMRRHHSGTDAL |
| | YFTGLEGAFGTVNPKYHPSRNNTIANFTFSAWVMPNANTNGFIIAKDDGNCSIYYCVKIQTNESHVT |
| | LSLHYKTLGSNATYIAKTTVMKYLEESVWLHLLIILEDGIIEFYLDGNAMPRGIKSLRGEAITDGPG |
| | ILRICAGINGNDRFTGLMQDVRSYERKLTLEEIYELHAMPAKSDLHPISGYLEFRQGETNKSFIISA |
| | RDDNDEEGEELFILKLVSVYGGARISEENTTARLTIQKSDNANGLFGFTGACIPEIAEEGSTISCVV |
| | ERTRCALDYVHVFYTISQIETDGINYLVDDFANASGTITFLPWQRSEVLNIYVLDDDIPELNEYFRV |
| | TLVSAIPGDGKLQSTPTSGASIDPEKETTDITIKASDHPYGLLQFSTGLPFQPKDAMTLPASSVPHI |
| | TVEEEDGEIRLLVIRAQGLLGRVTAEFRTVSLTAFSPEDYQNVAGTLEFQPCERYKYIFINITDNSI |
| | FELEKSFKVELLNLEGGVAELFRVDGSGSASLGVASQILVTIAASDHABGVFEFSPESLFVSGTEPE |
| | DGYSTVTLNVIRHUGTLSPVTLHWNIDSDPDGDLAFTSGNITFEIGQTSANITVEILPDEDPELDKA |
| | FSVSVLSVSSGSLGAHINATLTVLASDDPYGIFIFSEKNRPVKVEEATQNITLSIIRLKGLMGKVLV |
| | SYATLDDMEKPPYFPPNLARATQGRDYIPASGFALFGANQSEATIAISILDDDEPERSESVFIELLN |
| | STLVAKVQSRSIPNSPRLGPKVETIAQLIIIANDDAFGTLQLSAPIVRVAENUVGPIINVTRTGGAF |
| | ADVSVKFKAVPITAIAGEDYSIASSDVVLLEGETSKAVPIYVINDIYPELEESFLVQLMNETTGGAR |
| | LGALTEAVIIIEASDDPYGLFGFQITKLIVEEPEFNSVKVNLPIIRNSGTLCNVTVQWVATINGQLA |
| | TGDLRVVSGNVTFAPGETIQTLLLEVLADDVPEIEEVIQVQLTDASGGGTIGLDRIANIIIPANDDP |
| | YGTVAFAQMVYRVQEPLERSSCANITVRRSGGHFGRLLLFYSTSDIDVVALAMEECQDLLSYYESPI |
| | QGVPDPLWRTWMNVSAVGEPLYTCATLCLKEQACSAFSFFSASEGPQCFWMTSWISPAVNNSDFWTY |
| | RKNNTRVASLFSGQAVAGSDYEPVTRQWAIMQEGDEFANLTVSILPDDFPENDESFLISLLEVHLMN |
| | ISASLKNQPTIGQPNISTVVIALNGDAFGVFVIYNISFNTSEDCLFVEVQEQPQTLVELMIHRTGGS |
| | LGQVAVEWRVVGGTATEGLDFIGAGEILTFAEGETKKTVILTILDDSEPEDDESIIVSLVYTEGGSR |
| | ILPSSDTVRVNILANDNVAGIVSFQTASRSVIGHEGEILQFHVIRTFPGRGNVTVNWKIIGQNLELN |
| | FANFSGQLFFPEGSLNTTLFVHLLDDNIPEEKEVYQVILYDVRTQGVPPACIALLDAQCYAAVLTVE |
| | ASDEPHCVLNFALSSRFVLLQEANITIQLFINREFGSLGAINVTYPTVPGMLSLKNQTVGNLAEPEV |
| | DFVPIIGFLILEEGETAAAINITILEDDVPELEEYFLVNLTYVGLTMAASTSFPPRLGMRGFLFVSF |
| | CSLQMK |

Sequence comparison of the above protein sequences yields the following sequence relationships shown in Table 39B.

TABLE 39B
Comparison of NOV39a against NOV39b through NOV39d.

| | | Identities/ |
|---|---|---|
| | | Similarities for |
| | NOV39a Residues/ | the Matched |
| Protein Sequence | Match Residues | Region |
| NOV39b | 1 . . . 2741 | 2684/2741 (97%) |
| | 1 . . . 2741 | 2685/2741 (97%) |
| NOV39c | 1 . . . 1456 | 1442/1456 (99%) |
| | 1 . . . 1456 | 1443/1456 (99%) |
| NOV39d | 1 . . . 2753 | 2700/2753 (98%) |
| | 1 . . . 2753 | 2700/2753 (98%) |

Further analysis of the NOV39a protein yielded the following properties shown in Table 39C.

TABLE 39C
Protein Sequence Properties NOV39a

| | |
|---|---|
| PSort analysis: | 0.5050 probability located in cytoplasm; 0.3836 |
| | probability located in microbody (peroxisome); |
| | 0.1851 probability located in lysosome (lumen); |
| | 0.1000 probability located in mitochondrial |
| | matrix space |
| SignalP analysis: | No Known Signal Sequence Predicted |

A search of the NOV39a protein against the Geneseq database, a proprietary database that contains sequences published in patents and patent publication, yielded several homologous proteins shown in Table 39D.

TABLE 39D
Geneseq Results for NOV39a

| | | | Identities/ | |
|---|---|---|---|---|
| | | | Similarities | |
| Geneseq | Protein/Organism/Length | NOV39a Residues/ | for the | Expect |
| Identifier | [Patent #, Date] | Match Residues | Matched Region | Value |
| AAE10925 | Human monogenic | 1 . . . 2753 | 2736/2778 (98%) | 0.0 |
| | audiogenic | 1 . . . 2777 | 2739/2778 (98%) | |
| | seizure-susceptible-1 | | | |
| | (mass1) protein - Homo | | | |
| | sapiens, 2777 aa. | | | |
| | [WO200165927-A1, | | | |
| | 13 SEP. 2001] | | | |
| AAE10924 | Mouse monogenic | 1 . . . 2739 | 2295/2762 (83%) | 0.0 |
| | audiogenic | 1 . . . 2761 | 2516/2762 (91%) | |
| | seizure-susceptible-1 | | | |
| | (mass1) protein - Mus | | | |
| | musculus, 2780 aa. | | | |
| | [WO200165927-A1, | | | |
| | 13 SEP. 2001] | | | |
| AAE10949 | Mouse mass1 protein | 1 . . . 2049 | 1710/2072 (82%) | 0.0 |
| | mutant (7009deltaG) - Mus | 1 . . . 2071 | 1878/2072 (90%) | |
| | musculus, 2071 aa. | | | |
| | [WO200165927-A1, | | | |
| | 13 SEP. 2001] | | | |
| ABG61545 | Human transporter and ion | 1531 . . . 2288 | 740/758 (97%) | 0.0 |
| | channel, TRICH15, Incyte | 1 . . . 746 | 740/758 (97%) | |
| | ID 7476089CD1 - Homo | | | |
| | sapiens, 759 aa. | | | |
| | [WO200240541-A2, | | | |
| | 23 MAY. 2002] | | | |
| ABB05663 | Human signal transduction | 2232 . . . 2741 | 506/510 (99%) | 0.0 |
| | protein clone amy2_10p7 - | 9 . . . 518 | 507/510 (99%) | |
| | Homo sapiens, 1615 aa. | | | |
| | [WO200198454-A2, | | | |
| | 27 DEC. 2001] | | | |

In a BLAST search of public sequence datbases, the NOV39a protein was found to have homology to the proteins shown in the BLASTP data in Table 39E.

TABLE 39E
Public BLASTP Results for NOV39a

| | | NOV39a | Identities/ | |
|---|---|---|---|---|
| Protein | | Residues/ | Similarities | |
| Accession | | Match | for the | Expect |
| Number | Protein/Organism/Length | Residues | Matched Portion | Value |
| Q8WXG9 | Very large G protein-coupled | 1 . . . 2741 | 2721/2766 (98%) | 0.0 |
| | receptor 1b - Homo sapiens | 180 . . . 2945 | 2723/2766 (98%) | |
| | (Human), 6307 aa. | | | |
| Q91ZS2 | MASS1 - Mus musculus | 1 . . . 2739 | 2293/2762 (83%) | 0.0 |
| | (Mouse), 2780 aa. | 1 . . . 2761 | 2515/2762 (91%) | |
| Q8VHN7 | Very large G protein-coupled | 1 . . . 2741 | 2293/2764 (82%) | 0.0 |
| | receptor 1 - Mus musculus | 179 . . . 2941 | 2514/2764 (89%) | |
| | (Mouse), 6298 aa. | | | |
| Q91ZS1 | MASS1.2 - Mus musculus | 563 . . . 2739 | 1838/2192 (83%) | 0.0 |
| | (Mouse), 2238 aa. | 29 . . . 2219 | 2004/2192 (90%) | |
| Q8TF58 | KIAA1943 protein - Homo | 234 . . . 1273 | 1037/1050 (98%) | 0.0 |
| | sapiens (Human), 1054 aa | 1 . . . 1050 | 1037/1050 (98%) | |
| | (fragment). | | | |

### Example 40

The NOV40 clone was analyzed, and the nucleotide and encoded polypeptide sequences are shown in Table 40A.

TABLE 40A
NOV40 Sequence Analysis

| | SEQ ID NO: 167 | 2833 bp | |
|---|---|---|---|
| NOV40a, | CAAAGATCCAGTTTGGAAATGAGAGAGGACTAGCATGACACATTGGCTCCACCATTGATATCTCCCA | | |
| CG151014-01 | | | |
| DNA Sequence | GAGGTACAGAAACAGGATTCATGAAGATGTTGACAAGACTGCAAGTTCTTACCTTAGCTTTGTTTTC | | |
| | AAAGGGATTTTTACTCTCTTTAGGGGACCATAACTTTCTAAGGAGAGAGATTAAAATAGAAGGTGAC | | |
| | CTTGTTTTAGGGGGCCTGTTTCCTATTAACGAAAAAGGCACTGGAACTGAAGAATGTGGGCGAATCA | | |
| | ATGAAGACCGAGGGATTCAACGCCTGGAAGCCATGTTGTTTGCTATTGATGAAATCAACAAAGATGA | | |
| | TTACTTGCTACCAGGAGTGAAGTTGGGTGTTCACATTTTGGATACATGTTCAAGGGATACCTATGCA | | |
| | TTGGAGCAATCACTGGAGTTTGTCAGGGCATCTTTGACAAAAGTGGATGAAGCTGAGTATATGTGTC | | |
| | CTGATGGATCCTATGCCATTCAAGAAAACATCCCACTTCTCATTGCAGGGGTCATTGGTGGCTCTTA | | |
| | TAGCAGTGTTTCCATACAGGTGGCAAACCTGCTGCGGCTCTTCCAGATCCCTCAGATCAGCTACGCA | | |
| | TCCACCACCGCCAAACTCAGTGATAAGTCGCGCTATGATTACTTTGCCAGGACCGTGCCCCCCGACT | | |
| | TCTACCAGGCCAAAGCCATGGCTGAGATCTTGCGCTTCTTCAACTGGACCTACGTGTCCACAGTAGC | | |
| | CTCCGAGGGTGATTACGGGGAGACAGGGATCGAGGCCTTCGAGCAGGAAGCCCGCCTGCGCAACATC | | |
| | TGCATCGCTACGGCGGAGAAGGTGGGCCGCTCCAACATCCGCAAGTCCTACGACAGCGTGATCCGAG | | |
| | AACTGTTGCACAAGCCCAACGCGCGCGTCGTGGTCCTCTTCATCCGCAGCGACGACTCGCGGGAGCT | | |
| | CATTGCAGCCGCCAGCCGCGCCAATGCCTCCTTCACCTGGGTGGCCAGCGACGGCTGGGGCGCGCAG | | |
| | GAGAGCATCATCAAGGGCAGCGAGCATGTGGCCTACGGCGCCATCACCCTGGAGCTGGCCTCCCAGC | | |
| | CTGTCCGCCAGTTCGACCGCTACTTCCAGAGCCTCAACCCCTACAACAACCACCGCAACCCCTGGTT | | |
| | CCGGGACTTCTGGGAGCAAAAGTTTCAGTGCAGCCTCCAGAACAAACGCAACCACAGGCGCGTCTGC | | |
| | GACAAGCACCTGGCCATCGACAGCAGCAACTACGAGCAAGAGTCCAAGATCATGTTTGTGGTGAACG | | |
| | CGGTGTATGCCATGGCCCACGCTTTGCACAAAATGCAGCGCACCCTCTGTCCCAACACTACCAAGCT | | |
| | TTGTGATGCTATGAAGATCCTGGATGGGAAGAAGTTCTACAAGGATTACTTGCTGAAAATCAACTTC | | |
| | ACGGCTCCATTCAACCCAAATAAAGATGCAGATAGCATAGTCAAGTTTGACACTTTTGGAGATGGAA | | |
| | TGGGGCGATACAACGTGTTCAATTTCCAAAATGTAGGTGGAAAGTATTCCTACTTGAAAGTTGGTCA | | |
| | CTGGGCAGAAACCTTATCGCTAGATGTCAACTCTATCCACTGGTCCCCGAACTCAGTCCCCACTTCC | | |
| | CAGTGCAGCGACCCCTGTGCCCCCAATGAAATGAAGAATATGCAACCAGGGGATGTCTGCTGCTGGA | | |
| | TTTGCATCCCCTGTGAACCCTACGAATACCTGGCTGATGAGTTTACCTGTATGGATTGTGGGTCTGG | | |
| | ACAGTGGCCCACTGCAGACCTAACTGGATGCTATGACCTTCCTGAGGACTACATCAGGTGGGAAGAC | | |
| | GCCTGGGCCATTGGCCCAGTCACCATTGCCTGTCTGGGTTTTATGTGTACATGCATGGTTGTAACTG | | |
| | TTTTTATCAAGCACAACAACACACCCTTGGTCAAAGCATCCGGCCGAGAACTCTGCTACATCTTATT | | |
| | GTTTGGGGTTGGCCTGTCATACTGCATGACATTCTTCTTCATTGCCAAGCCATCACCAGTCATCTGT | | |
| | GCATTGCGCCGACTCGGGCTGGGGAGTTCCTTCGCTATCTGTTACTCAGCCCTGCTGACCAAGACAA | | |
| | ACTGCATTGCCCGCATCTTCGATGGGGTCAAGAATGGCGCTCAGAGGCCAAAATTCATCAGCCCCAG | | |
| | TTCTCAGGTTTTCATCTGCCTGGGTCTGATCCTGGTGCAAATTGTGATGGTGTCTGTGTGGCTCATC | | |
| | CTGCAGGCCCCAGGCACCAGGAGGTATACCCTTACAGAGAAGCGGGAAACAGTCATCCTAAAATGCA | | |
| | ATGTCAAAGATTCCAGCATGTTGATCTCTCTTACCTACGATGTGATCCTGGTGATCTTATGCACTGT | | |
| | GTACGCCTTCAAAACGCGGAAGTGCCCAGAAAATTTCAACGAAGCTAAGTTCATAGGTTTTACCATG | | |
| | TACACCACGTGCATCATCTGGTTGGCCTTCCTCCCTATATTTTATGTGACATCAAGTGACTACAGAC | | |
| | CTCTGCAAGCACGTATGTGTCAACGGTGTGCAATGGGCGGGAAGTCCTCGACTCCACCACCTCATCT | | |
| | CTGTGATTGTGAATTGCAGTTCAGTTCTTGTGTTTTTAGACTGTTAGACAAAAGTGCTCACGTGCAG | | |
| | CTCCAGAATATGGAAACAGAGCAAAAGAACAACCCTAGTACCTTTTTTTAGAAACAGTACGATAAAT | | |
| | TATTTTTGAGGACTGTATATAGTGATGTGCTAGAACTTTCTAGGCTGAGTCTAGTGCCCCTATTATT | | |
| | AACAATTCCCCCAGAACATGGAAATAACCATTGTTTACAGAGCTCAGCATTGGTGACAGGGTCTGAC | | |
| | ATGGTCAGTCTACTTCAAG | | |
| | ORF Start: ATG at 88 | | ORF Stop: TAG at 2662 |
| | SEQ ID NO: 168 | 858 aa | MW at 96975.6 kD |
| NOV40a, | MKMLTRLQVLTLALFSKGFLLSLGDHNFLRREIKIEGDLVLGGLFPINEKGTGTEECGRTNEDRGIQ | | |
| CG151014-01 | | | |
| Protein Sequence | RLEAMLFAIDEINKDDYLLPGVKLGVHTLDTCSRDTYALEQSLEFVRASLTKVDEAEYMCPDGSYAI | | |
| | QENIPLLIAGVIGGSYSSVSIQVANLLRLFQIPQISYASTSAKLSDKSRYDYFARTVPPDFYQAKAM | | |
| | AEILRFFNWTYVSTVASEGDYGETGIEAFEQEARLRNICIATAEKVGRSNIRKSYDSVIRELLQKPN | | |
| | ARVVVLFNRSDDSRELIAAASRANASFTWVASDGWGAQESIIKGSEHVAYGAITLELASQPVRQFDR | | |
| | YFQSLNPYNNHRNPWFRDFWEQKFQCSLQNKRNHRRVCDKHLAIDSSNYEQESKIMFVVNAVYAMAH | | |
| | ALHKMQRTLCPNTTKLCDAMKILDGKKLYKDYLLKINFTAPFNPNKDADSIVKFDTFGDGMGRYNVF | | |
| | NFQNVGGKYSYLKVGHWAETLSLDVNSIHWSRNSVFTSQCSDPCAPNEMXNMQPGDVCCWICIPCEP | | |
| | YEYLADEFTCMDCGSGQWPTADLTGCYDLPEDYIRWEDAWAIGPVTIACLGFMCTCMVVTVEIKHNN | | |
| | TPLVKASGRELCYILLFGVGLSYCMTFFFIAKPSPVICALRRLGLGSSFAICYSALLTKTNCIARIF | | |
| | DGVKNGAQRPKFISPSSQVFICLGLILVQIVNVSVWLILEAPGTRRYTLTEKRETVILKCNVKDSSM | | |
| | LISLTYDVILVILCTVYAFKTRKCPENFNEAXFIGFTMYTTCIIWLAFLPIFYVTSSDYRPLQARMC | | |
| | QRCAMGGKSSTPPPHLCDCELQFSSCVFRLLDKSAHVQLQNMETEQKNNPSTFF | | |
| | SEQ ID NO: 169 | 1758 bp | |
| NOV40b, | CAAAGATCCAGTTTGGAAATGAGAGAGGACTAGCATGACACATTGGCTCCACCATTGATATCTCCCA | | |
| CG151014-02 | | | |
| DNA Sequence | GAGGTACAGAAACAGGATTCATGAAGATGTTGACAAGACTGCAAGTTCTTACCTTAGCTTTGTTTTC | | |
| | AAAGGGATTTTTACTCTCTTTAGGGGACCATAACTTTCTAAGGAGAGAGATTAAAATAGAAGGTGAC | | |
| | CTTGTTTTAGGGGGCCTGTTTCCTATTAACGAAAAAGGCACTGGAACTGAAGAATGTGGGCGAATCA | | |
| | ATGAAGACCGAGGGATTCAACGCCTGGAAGCCATGTTGTTTGCTATTGATGAAATCAACAAAGATGA | | |
| | TTACTTGCTACCAGGAGTGAAGTTGGGTGTTCACATTTTGGATACATGTTCAACGGATACCTATGCA | | |
| | TTGGAGCAATCACTGGAGTTTGTCAGGGCATCTTTGACAAAAGTGGATGAAGCTGAGTATATGTGTC | | |
| | CTGATGGATCCTATGCCATTCAAGAAAACATCCCACTTCTCATTGCAGGGGTCATTGGTGGCTCTTA | | |
| | TAGCAGTGTTTCCATACAGGTGGCAAACCTGCTGCGGCTCTTCCAGATCCCTCAGATCAGCTACGCA | | |
| | TCCACCAGCGCCAAACTCAGTGATAAGTCGCGCTATGATTACTTTGCCAGGACCGTGCCCCCCGACT | | |
| | TCTACCAGGCCAAAGCCATGGCTGAGATCTTGCGCTTCTTCAACTGGACCTACGTGTCCACAGTAGC | | |
| | CTCCGAGGGTGATTACGGGGAGACAGGGATCGAGGCCTTCGAGCAGGAAGCCCGCCTGCGCAACATC | | |
| | TGCATCGCTACGGCGGAGAAGGTGGGCCGCTCCAACATCCGCAAGTCCTACGACAGCGTGATCCGAG | | |
| | AACTGTTGCAGAAGCCCAACGCGCGCGTCGTCGTCCTCTTCATCCGCAGCGACGACTCGCGGGAGCT | | |
| | CATTGCAGCCGCCAGCCGCGCCAATGCCTCCTTCACCTGGGTGCCCAGCGACGGCTGCGGCGCGCAG | | |
| | GAGAGCATCATCAAGGGCAGCGAGCATGTGGCCTACGGCCCCATCACCCTGGAGCTGGCCTCCCAGC | | |
| | CTGTCCGCCAGTTCGACCCCTACTTCCAGAGCCTCAACCCCTACAACAACCACCGCAACCCCTGGTT | | |
| | CCGGGACTTCTGCGAGCAAAAGTTTCACTGCAGCCTCCAGAACAAACGCAACCACAGGCGCGTCTGC | | |
| | GACAAGCACCTGGCCATCGACAGCAGCAACTACGAGCAAGAGTCCAAGATCATGTTTGTGGTGAACG | | |
| | CGGTGTATGCCATCGCCCACGCTTTGCACAAAATGCAGCGCACCCTCTGTCCCAACACTACCAAGCT | | |
| | TTGTGATGCTATGAAGATCCTGGATGGGAAGAAGTTGTACAAGGATTACTTGCTGAAAATCAACTTC | | |
| | ACGGGTGCAGACGACAACCATGTGCATCTCCGTCAGCCTGAGTGGCTTTCTGGTCTTGGGCTGTTTG | | |
| | TTTGCACCCAAGGTTCACATCATCCTGTTTCAACCCCAGAAGAATGTTGTCACACACAGACTGCACC | | |
| | TCAACAGGTTCAGTGTCAGTGGAACTGGGACCACATACTCTCAGTCCTCTGCAAGCACGTATGTGCC | | |
| | AACGGTGTGCAATGGGCGGGAAGTCCTCGACTCCACCACCTCATCTCTGTGATTGTGAATTGCAGTT | | |
| | CAGTTCTTGTGTTTTTAGACTGTTAGACAAAAGTGCTCACGTGCACCTCCAGAATATGGAAACAGAG | | |
| | CAAAAGAACAACCCTA | | |
| | ORF Start: ATG at 88 | | ORF Stop: TAG at 1699 |
| | SEQ ID NO: 170 | 537 aa | MW at 60801.8 kD |
| NOV40b, | MKMLTRLQVLTLALFSKGELLSLGDHNFLRREIKIEGDLVLGGLFPINEKGTGTEECGRINEDRGIQ | | |
| CG151014-02 | | | |
| Protein Sequence | RLEAMLFAIDEINKDDYLLPGVKLGVHILDTCSRDTYALEQSLEFVRASLTKVDEAEYMCPDGSYAI | | |
| | QENIPLLIAGVIGGSYSSVSIQVANLLRLFQIPQISYASTSAKLSDKSRYDYFARTVPPDFYQAKAM | | |
| | AEILRFFNWTYVSTVASEGDYGETGIEAFEQEARLRNICIATAEKVGRSNIRKSYDSVIRELLQKPN | | |
| | ARVVVLFMRSDDSRELIAAASRANASFTWVASDGWGAQESIIKGSEHVAYGAITLELASQPVRQFDR | | |
| | YFQSLNPYNNHRNPWFRDFWEQKFQCSLQNKRNHRRVCDKHLAIDSSNYEQESKIMFVVNAVYAMAH | | |
| | ALHKMQRTLCPNTTKLCDAMKILDGKKLYKDYLLKINFTGADDNHVHLRQPEWLCGLGLFVCTQGSH | | |
| | HPVSTPEECCHTQTAFQQVQCQWNWDHILSVLCKHVCANGVQWAGSPRLHHLISVIVNCSSVLVFLD | | |
| | C | | |
| | SEQ ID NO: 171 | 1758 bp | |
| NOV40c, | CCTTGATCCAGTTTCCAAATGAGAGAGGACTAGCATGACACATTGGCTCCACCATTGATATCTCCCA | | |
| CG151014-03 | | | |
| DNA Sequence | CAGGTACAGAAACAGGATTCATGAAGATGTTGACAAGACTGCAAGTTCTTACCTTAGCTTTGTTTTC | | |
| | AAAGGGATTTTTACTCTCTTTAGGGGACCATAACTTTCTAAGGAGAGACATTAAAATAGAAGGTGAC | | |
| | CTTGTTTTAGGGGGCCTGTTTCCTATTAACGAAAAAGGCACTGGAACTGAAGAATGTGGGCGAATCA | | |
| | ATGAAGACCGAGGGATTCAACGCCTGGAAGCCATGTTGTTTGCTATTGATGAAATCAACAAAGATGA | | |
| | TTACTTGCTACCAGGAGTGAAGTTGGGTGTTCACATTTTGGATACATGTTCAAGGGATACCTATGCA | | |
| | TTGCAGCAATCACTGGAGTTTGTCAGGGCATCTTTGACAAAAGTGGATGAAGCTGAGTATATGTGTC | | |
| | CTGATGGATCCTATGCCATTCAAGAAAACATCCCACTTCTCATTGCAGGGGTCATTGGTGGCTCTTA | | |
| | TAGCAGTGTTTCCATACAGGTGGCAAACCTGCTGCGGCTCTTCCAGATCCCTCAGATCAGCTACGCA | | |
| | TCCACCAGCGCCAAACTCAGTGATAAGTCGCGCTATGATTACTTTGCCAGGACCGTGCCCCCCGACT | | |
| | TCTACCAGGCCAAACCCATGGCTGAGATCTTGCGCTTCTTCAACTGGACCTACGTGTCCACAGTAGC | | |
| | CTCCGAGGGTGATTACGGGCAGACAGGGATCGAGGCCTTCGAGCAGGAAGCCCGCCTGCGCAACATC | | |
| | TGCATCGCTACGGCGGAGAAGGTGGGCCGCTCCAACATCCGCAAGTCCTACGACAGCGTGATCCGAG | | |
| | AACTGTTGCAGAAGCCCAACGCGCGCGTCGTGGTCCTCTTCATGCGCAGCGACGACTCGCGGGAGCT | | |
| | CATTGCAGCCGCCAGCCGCGCCAATGCCTCCTTCACCTGGGTGGCCAGCGACGCCTGGGGCGCGCAG | | |
| | GAGACCATCATCAAGGGCAGCGAGCATGTGGCCTACGGCGCCATCACCCTGGAGCTGGCCTCCCAGC | | |
| | CTGTCCGCCAGTTCGACCGCTACTTCCAGAGCCTCAACCCCTACAACAACCACCGCAACCCCTGGTT | | |
| | CCGGGACTTCTGGGAGCAAAAGTTTCAGTGCAGCCTCCAGAACAAACGCAACCACAGGCGCGTCTGC | | |
| | GACAAGCACCTGGCCATCGACAGCAGCAACTACGAGCAAGAGTCCAAGATCATGTTTGTGGTGAACG | | |
| | CGGTGPATGCCATGGCCCACGCTTTGCACAAAATGCAGCCCACCCTCTGTCCCAACACTACCAAGCT | | |
| | TTGTGATGCTATGAAGATCCTGGATGGGAAGAAGTTGTACAAGGATTACTTGCTGAAAATCAACTTC | | |
| | ACGGGTGCAGACGACAACCATGTGCATCTCCGTCAGCCTGAGTGGCTTTGTGGTCTTGGGCTGTTTG | | |
| | TTTCCACCCAAGGTTCACATCATCCTGTTTCAACCCCAGAAGAATGTTGTCACACACAGACTGCACC | | |
| | TCAACAGGTTCAGTGTCAGTGGAACTGGGACCACATACTCTCAGTCCTCTGCAAGCACGTATGTGCC | | |
| | AACGGTGTGCAATGGGCGGGAAGTCCTCGACTCCACCACCTCATCTCTGTGATTGTGAATTGCAGTT | | |
| | CAGTTCTTGTGTTTTTAGACTGTTAGACAAAAGTGCTCACGTGCAGCTCCACAATATGGAAACAGAG | | |
| | CAAAAGAACAACCCTA | | |
| | ORF Start: ATG at 88 | | ORF Stop: TAG at 1699 |
| | SEQ ID NO: 172 | 537 aa | MW at 60801.8kD |

| | |
|---|---|
| NOV40c, | MKMLTRLQVLTLALFSKCFLLSLGDHNFLRREIKIEGDLVLGGLFPINEKGTGTEECGRINEDRGIQ |
| CG151014-03 | |
| Protein Sequence | RLEAMLFAIDEINKDDYLLPGVKLGVHTLDTCSRDTYALEQSLEFVRASLTKVDEAEYMCPDGSYAI |
| | QENIPLLIAGVIGGSYSSVSIQVANLLRLFQIPQISYASTSAKLSDKSRYDYFARTVPPDFYQAXAN |
| | AEILRFFNWTYVSTVASEGDYGETGIEAFEQEARLRNICIATAEKVGRSNIRKSYDSVIRELLQKPN |
| | ARVVVLFMRSDDSRELIAAASRANASFTWVASDGWGAQESIIKGSEHVAYGAITLELASQPVRQFDR |
| | YFQSLNPYNNHRNPWFRDFWEQKFQCSLQNKRNHRRVCDKHLAIDSSNYEQESKIMFVVNAVYAMAH |
| | ALHKMQRTLCPNTTKLCDAMKILDGKKLYKDYLLKINFTGADDNHVHLRQPEWLCGLGLFVCTQGSH |
| | HPVSTPEECCHTQTAPQQVQCQWNWDHILSVLCKHVCANGVQWAGSPRLHHLISVIVNCSSVLVFLD |
| | C |

Sequence comparison of the above protein sequences yields the following sequence relationships shown in Table 40B.

TABLE 40B
Comparison of NOV40a against NOV40b and NOV40c.

| | | Identities/ |
|---|---|---|
| | NOV40a Residues/ | Similarities for |
| Protein Sequence | Match Residues | the Matched Region |
| NOV40b | 1 . . . 441 | 409/441 (92%) |
| | 1 . . . 441 | 409/441 (92%) |
| NOV40c | 1 . . . 441 | 409/441 (92%) |
| | 1 . . . 441 | 409/441 (92%) |

Further analysis of the NOV40a protein yielded the following properties shown in Table 40C.

TABLE 40C
Protein Sequence Properties NOV40a

| | |
|---|---|
| PSort | 0.6400 probability located in plasma membrane; 0.4600 |
| analysis: | probability located inGolgi body; 0.3700 probability |
| | located in endoplasmic reticulum (membrane);0.1000 |
| | probability located in endoplasmic reticulum (lumen) |
| SignalP | Cleavage site between residues 25 and 26 |
| analysis: | |

A search of the NOV40a protein against the Geneseq database, a proprietary database that contains sequences published in patents and patent publication, yielded several homologous proteins shown in Table 40D.

TABLE 40D
Geneseq Results for NOV40a

| | | | Identities/ | |
|---|---|---|---|---|
| | | | Similarities for | |
| Geneseq | Protein/Organism/Length | NOV40a Residues/ | the Matched | Expect |
| Identifier | [Patent #, Date] | Match Residues | Region | Value |
| AAE15990 | Human glutamate receptor, | 3 . . . 811 | 797/809 (98%) | 0.0 |
| | metabotrophic 3 (GRM3) | 1 . . . 809 | 799/809 (98%) | |
| | protein - Homo sapiens, 877 | | | |
| | aa. [WO200196350-A2, | | | |
| | 20 DEC. 2001] | | | |
| AAR82657 | Human mGluR3 - Homo | 3 . . . 811 | 797/809 (98%) | 0.0 |
| | sapiens, 877 aa. | 1 . . . 809 | 799/809 (98%) | |
| | [WO9522609-A2, | | | |
| | 24 AUG. 1995] | | | |
| AAM23698 | Human EST encoded protein | 1 . . . 811 | 796/811 (98%) | 0.0 |
| | SEQ ID NO: 1223 - Homo | 1 . . . 811 | 798/811 (98%) | |
| | sapiens, 857 aa. | | | |
| | [WO200154477-A2, | | | |
| | 02 AUG. 2001] | | | |
| AAR64252 | Human mGluR3 - Homo | 1 . . . 811 | 796/811 (98%) | 0.0 |
| | sapiens, 879 aa. | 1 . . . 811 | 799/811 (98%) | |
| | [WO9429449-A, | | | |
| | 22 DEC. 1994] | | | |
| AAO15105 | Human | 21 . . . 811 | 777/791 (98%) | 0.0 |
| | ph2SPMGluR3-CaR*AAA* | 17 . . . 807 | 781/791 (98%) | |
| | Gqi5 fusion construct protein | | | |
| | sequence - Chimeric - Homo | | | |
| | sapiens, 1402 aa. | | | |
| | [WO200229033-A2, | | | |
| | 11 APR. 2002] | | | |

In a BLAST search of public sequence datbases, the NOV40a protein was found to have homology to the proteins shown in the BLASTP data in Table 40E.

TABLE 40E
Public BLASTP Results for NOV40a

| | | | Identities/ | |
|---|---|---|---|---|
| Protein | | | Similarities for | |
| Accession | | NOV40a Residues/ | the Matched | Expect |
| Number | Protein/Organism/Length | Match Residues | Portion | Value |
| Q14832 | Metabotropic glutamate | 3 . . . 811 | 797/809 (98%) | 0.0 |
| | receptor 3 precursor | 1 . . . 809 | 799/809 (98%) | |
| | (mGluR3) - Homo sapiens | | | |
| | (Human), 877 aa. | | | |
| Q8TBH9 | Glutamate receptor, | 3 . . . 811 | 795/809 (98%) | 0.0 |
| | metabotropic 3 - Homo | 1 . . . 809 | 797/809 (98%) | |
| | sapiens (Human), 877 aa. | | | |
| Q9QYS2 | Metabotropic glutamate | 1 . . . 811 | 773/811 (95%) | 0.0 |
| | receptor 3 protein - Mus | 1 . . . 811 | 792/811 (97%) | |
| | musculus (Mouse), 879 aa. | | | |
| P31422 | Metabotropic glutamate | 1 . . . 811 | 772/811 (95%) | 0.0 |
| | receptor 3 precursor - Rattus | 1 . . . 811 | 790/811 (97%) | |
| | norvegicus (Rat), 879 aa. | | | |
| JC7160 | metabotropic glutamate | 1 . . . 811 | 771/811 (95%) | 0.0 |
| | receptor subtype 3 precursor - | 1 . . . 811 | 790/811 (97%) | |
| | mouse, 879 aa. | | | |

PFam analysis predicts that the NOV40a protein contains the domains shown in the Table 40F.

TABLE 40F
Domain Analysis of NOV40a

| | | Identities/ | |
|---|---|---|---|
| | | Similarities | |
| | NOV40a Match | for the | Expect |
| Pfam Domain | Region | Matched Region | Value |
| ANF_receptor | 58 . . . 489 | 194/473 (41%) | 3.2e−173 |
| | | 399/473 (84%) | |
| 7tm_3 | 576 . . . 820 | 109/283 (39%) | 3.1e−104 |
| | | 217/283 (77%) | |

### Example 41

The NOV41 clone was analyzed, and the nucleotide and encoded polypeptide sequences are shown in Table 41A.

TABLE 41A
NOV41 Sequence Analysis

| | SEQ ID NO: 173 | 880 bp | |
|---|---|---|---|
| NOV41a, | GAATTCTGATGTGCTTCAGTCCACAGAACAGTAACACATGAGCTGCTTTTGGGGAGAGCTTGAGTAC | | |
| CG151297-01 | | | |
| DNA Sequence | TCAGTCGGAGCATCATCATGGGGTCTAGTGCCACAGAGATTGAAGAATTGGAAAACACCACTTTTAA | | |
| | GTATCTTACAGGAGAACAGACTCAAAAAATGTGGCAGCGCCTGAAAGGAATACTAAGATGCTTGGTG | | |
| | AAGCAGCTGGAAAGAGGTGATGTTAACGTCGTCGACTTAAAGAAGAATATTGAATATGCGGCATCTG | | |
| | TGCTGGAAGCAGTTTATATCGATGAAACAAGAAGACTTCTGGATACTGAAGATGAGCTCAGTGACAT | | |
| | TCAGACTGACTCAGTCCCATCTGAAGTCCGGGACTCGTTGGCTTCTACCTTTACACGGAAAATGGGG | | |
| | ATGACAAAAAAGAAACCTGAGGAAAAACCAAAATTTCGGAGCATTGTGCATGCTGTTCAAGCTGGAA | | |
| | TTTTTGTGGAAAGAATGTACCGAAAAACATTTTCTCTTCTGACAGACTCAACAGAGAAAATTGTTAT | | |
| | TCCTCTTATAGAGGAAGCCTCAAAAGCCGAAACTTCTTCCTATGTGGCAAGCAGCTCAACCACCATT | | |
| | GTGGGGTTACACATTGCTGATGCACTAAGACGATCAAATACAAAAGGCTCCATGAGTGATGGGTCCT | | |
| | ATTCCCCAGACTACTCCCTTGCAGCAGTGGACCTGAAGAGTTTCAAGAACAACCTGGTGGACATCAT | | |
| | TCAGCAGAACAAAGAGAGGTGGAAAGAGTTAGCTGCACAAGAAGCAAGAACCAGTTCACAGAAGTGT | | |
| | GAGTTTATTCATCAGTAAACACCTTTAAGTAAAACCTCGTGCATGGTGGCAGCTCTAATTTGACCAA | | |
| | AAGACTTGG | | |
| | ORF Start: ATG at 85 | | ORF Stop: TAA at 820 |
| | SEQ ID NO: 174 | 245 aa | MW at 27787.2 kD |
| NOV41a, | MGSSATEIEELENTTFKYLTGEQTEKMWQRLKGILRCLVKQLERGDVNVVDLKKNIEYAASVLEAVY | | |
| CG151297-01 | | | |
| Protein Sequence | IDETRRLLDTEDELSDIQTDSVPSEVRDWLASTFTRKMGMTKKKPEEKPKFRSIVHAVQAGIFVERN | | |
| | YRKTFSLLTDSTEKIVIPLIEEASKAETSSYVASSSTTIVGLHIADALRRSNTKGSMSDGSYSPDYS | | |
| | LAAVDLKSFKNNLVDIIQQNKERWKELAAQEARTSSQKCEFIHQ | | |
| | SEQ ID NO: 175 | 1817 bp | |
| NOV41b, | TCAGTGCACAGAACAGTAACAGATGAGCTGCTTTTGGCGAGAGCTTGAGTACTCAGTCGGTCAGTAG | | |
| CG151297-02 | | | |
| DNA Sequence | TACAGTAGCAGGCTCACATGTACGGATTGTTCTTGTGAGGAGCATCATCATGGGGTCTAGTGCCACA | | |
| | GAGATTGAAGAATTGGAAAACACCACTTTTAAGTATCTTACAGGAGAACAGACTGAAAAAATGTGGC | | |
| | AGCGCCTGAAAGGAATACTAAGATGCTTGGTGAAGCAGCTGGAAACACGTGATGTTAACGTCGTCGA | | |
| | CTTAAAGAAGAATATTGAATATGCGGCATCTGTGCTGGAAGCAGTTTATATCGATGAAACAAGAAGA | | |
| | CTTCTGGATACTGAAGATGACCTCAGTGACATTCAGACTGACTCAGTCCCATCTGAAGTCCGGGACT | | |
| | GGTTGGCTTCTACCTTTACACGGAAAATGGGGATGACAAAAAAGAAACCTGAGGAAAAACCAAAATT | | |
| | TCGGAGCATTGTGCATGCTGTTCAAGCTGGAATTTTTGTGGAAAGAATGTACCGAAAAACATATCAT | | |
| | ATGGTTGGTTTGGCATATCCAGCAGCTGTCATCGTAACATTAAAGGATGTTGATAAATGGTCTTTCG | | |
| | ATGTATTTGCCCTAAATGAAGCAAGTGGAGAGCATAGTCTGAAGTTTATGATTTATGAACTGTTTAC | | |
| | CAGATATGATCTTATCAACCGTTTCAAGATTCCTGTTTCTTGCCTAATCACCTTTGCAGAAGCTTTA | | |
| | GAAGTTGGTTACGGCAAGTACAAAAATCCATATCACAATTTGATTCATGCAGCTGATGTCACTCAAA | | |
| | CTGTGCATTACATAATGCTTCATACAGGTATCATGCACTGGCTCACTGAACTGGAAATTTTAGCAAT | | |
| | GGTCTTTGCTGCTGCCATTCATGATTATGAGCATACAGCGACAACAAACAACTTTCACATTCAGACA | | |
| | AGGTCAGATGTTGCCATTTTGTATAATCATCGCTCTGTCCTTGAGAATCACCACGTGAGTGCAGCTT | | |
| | ATCGACTTATGCAAGAAGAAGAAATGAATATCTTGATAAATTTATCCAAAGATCACTGGAGGGATCT | | |
| | TCGGAACCTAGTGATTGAAATGGTTTTATCTACAGACATGTCAGGTCACTTCCAGCAAATTAAAAAT | | |
| | ATAAGAAACAGTTTGCAGCAGCCTGAAGGGATTGACAGAGCCAAAACCATGTCCCTGATTCTCCACG | | |
| | CAGCAGACATCAGCCACCCAGCCAAATCCTGGAAGCTGCATTATCGGTGGACCATGGCCCTAATGGA | | |
| | GGAGTTTTTCCTGCAGGGAGATAAAGAAGCTGAATTAGGGCTTCCATTTTCCCCACTTTGTGATCGG | | |
| | AAGTCAACCATGGTGGCCCAGTCACAAATAGGTTTCATCGATTTCATAGTAGAGCCAACATTTTCTC | | |
| | TTCTGACAGACTCAACAGAGAAAATTCTTATTCCTCTTATAGAGGAAGCCTCAAAAGCCGAAACTTC | | |
| | TTCCTATGTGGCAAGCAGCTCAACCACCATTGTGGGGTTACACATTGCTGATGCACTAAGACGATCA | | |
| | AATACAAAAGGCTCCATGAGTGATGGGTCCTATTCCCCAGACTACTCCCTTGCAGCAGTGGACCTGA | | |
| | AGAGTTTCAAGAACAACCTGGTGGACATCATTCAGCAGAACAAAGAGAGGTGGAAACAGTTAGTTGC | | |
| | ACAAGAAGCAAGAACCAGTTCACAGAAGTGTGAGTTTATTCATCAGTAAACACCTTTAAGTAAAACC | | |
| | TCGTGCATGGTGGCAGCTCTAATTTGACCAAAAGACTTGGAGATTTTGATTATGCTTGCTGGATATC | | |
| | TATTCTGT | | |
| | ORF Start: ATG at 117 | | ORF Stop: TAA at 1722 |
| | SEQ ID NO: 176 | 535 aa | MW at 61249.3 kD |

| | |
|---|---|
| NOV41b, | MGSSATEIEELENTTFKYLTGEQTEKMWQRLKGILRCLVKQLERGDVNVVDLKKNIEYAASVLEAVY |
| CG151297-02 | |
| Protein Sequence | IDETRRLLDTEDELSDIQTDSVPSEVRDWLASTFTRKMGMTKKKPEEKPKFRSIVHAVQAGIFVERM |
| | YRKTYHMVGLAYPAAVIVTLKDVDKWSFDVFALNEASGEHSLKFMIYELFTRYDLINRFKIPVSCLI |
| | TFAEALEVGYGKYKNPYHNLIHAADVTQTVHYIMLHTGIMHWLTELEILAMVFAAAIHDYEHTGTTN |
| | NFHIQTRSDVAILYNDRSVLENHHVSAAYRLMQEEEMNILINLSKDDWRDLRNLVIEMVLSTDMSGH |
| | FQQIKNIRNSLQQPEGIDRAKTMSLTLHAADISHPAKSWKLHYRWTMALMEEFFLQGDKEAELGLPF |
| | SPLCDRKSTMVAQSQIGFIDFIVEPTFSLLTDSTEKIVIPLIEEASKAETSSYVASSSTTIVGLHIA |
| | DALRRSNTKGSMSDGSYSPDYSLAAVDLKSFKNNLVDIIQQNKERWKELVAQEARTSSQKCEFIHQ |

Sequence comparison of the above protein sequences yields the following sequence relationships shown in Table 41B.

TABLE 41B
Comparison of NOV41a against NOV41b.

| | | Identities/ |
|---|---|---|
| Protein | NOV41a Residues/ | Similarities for |
| Sequence | Match Residues | the Matched Region |
| NOV41b | 1 . . . 159 | 141/159 (88%) |
| | 1 . . . 159 | 148/159 (92%) |

Further analysis of the NOV41a protein yielded the following properties shown in Table 41C.

TABLE 41C
Protein Sequence Properties NOV41a

| | |
|---|---|
| PSort | 0.8800 probability located in nucleus; 0.1000 probability |
| analysis: | located inmitochondrial matrix space; 0.1000 probability |
| | located in lysosome (lumen);0.1000 probability located |
| | in plasma membrane |
| SignalP | No Known Signal Sequence Predicted |
| analysis: | |

A search of the NOV41a protein against the Geneseq database, a proprietary database that contains sequences published in patents and patent publication, yielded several homologous proteins shown in Table 41D.

TABLE 41D
Geneseq Results for NOV41a

| | | | Identities/ | |
|---|---|---|---|---|
| | | | Similarities for | |
| Geneseq | Protein/Organism/Length | NOV41a Residues/ | the Matched | Expect |
| Identifier | [Patent #, Date] | Match Residues | Region | Value |
| AAB85116 | Human 3′, 5′ cyclic | 1 . . . 159 | 141/159 (88%) | 5e−75 |
| | nucleotide phosphodiesterase | 1 . . . 159 | 148/159 (92%) | |
| | (HSPDE1A3A) - Homo | | | |
| | sapiens, 535 aa. | | | |
| | [EP1097707-A1, | | | |
| | 09 MAY 2001] | | | |
| AAB85105 | Human 3′, 5′ cyclic | 1 . . . 159 | 141/159 (88%) | 5e−75 |
| | nucleotide phosphodiesterase | 1 . . . 159 | 148/159 (92%) | |
| | (HSPDE1A3A) - Homo | | | |
| | sapiens, 535 aa. | | | |
| | [EP1097706-A1, | | | |
| | 09 MAY 2001] | | | |
| AAE07953 | Human phosphodiesterase | 1 . . . 159 | 141/159 (88%) | 5e−75 |
| | (PDE) type 1 protein - Homo | 1 . . . 159 | 148/159 (92%) | |
| | sapiens, 535 aa. | | | |
| | [EP1097719-A1, | | | |
| | 09 MAY 2001] | | | |
| AAE07917 | Human phosphodiesterase | 1 . . . 159 | 141/159 (88%) | 5e−75 |
| | (PDE) type 1 protein - Homo | 1 . . . 159 | 148/159 (92%) | |
| | sapiens, 535 aa. | | | |
| | [EP1097718-A1, | | | |
| | 09 MAY 2001] | | | |
| AAY80988 | Human 61 kD CaM-PDE | 1 . . . 159 | 141/159 (88%) | 5e−75 |
| | (clone pHcam61-6N-7), SEQ | 1 . . . 159 | 148/159 (92%) | |
| | ID NO: 49 - Homo sapiens, | | | |
| | 535 aa. [US6015677-A, | | | |
| | 18 JAN. 2000] | | | |

In a BLAST search of public sequence datbases, the NOV41a protein was found to have homology to the proteins shown in the BLASTP data in Table 41E.

TABLE 41E
Public BLASTP Results for NOV41a

| | | | Identities/ | |
|---|---|---|---|---|
| Protein | | | Similarities for | |
| Accession | | NOV41a Residues/ | the Matched | Expect |
| Number | Protein/Organism/Length | Match Residues | Portion | Value |
| AAH22480 | Hypothetical 62.3 kDa protein - | 1 . . . 159 | 141/159 (88%) | 1e−74 |
| | Homo sapiens (Human), 545 | 1 . . . 159 | 148/159 (92%) | |
| | aa. | | | |
| P54750 | Calcium/calmodulin-dependent | 2 . . . 159 | 140/158 (88%) | 6e−74 |
| | 3′,5′-cyclic nucleotide | 1 . . . 158 | 147/158 (92%) | |
| | phosphodiesterase 1A (EC | | | |
| | 3.1.4.17) (Cam-PDE 1A) (61 | | | |
| | kDa Cam-PDE) (hCam-1) - | | | |
| | Homo sapiens (Human), 534 | | | |
| | aa. | | | |
| Q9EPR9 | Phosphodiesterase 1A - Rattus | 1 . . . 159 | 134/159 (84%) | 6e−71 |
| | norvegicus (Rat), 542 aa. | 1 . . . 159 | 144/159 (90%) | |
| Q61481 | Calcium/calmodulin-dependent | 1 . . . 159 | 133/159 (83%) | 3e−70 |
| | 3′,5′-cyclic nucleotide | 21 . . . 179 | 143/159 (89%) | |
| | phosphodiesterase 1A (EC | | | |
| | 3.1.4.17) (Cam-PDE 1A) (61 | | | |
| | kDa Cam-PDE) - Mus | | | |
| | musculus (Mouse), 565 aa. | | | |
| A45334 | 3′,5′-cyclic-nucleotide | 1 . . . 159 | 129/159 (81%) | 6e−69 |
| | phosphodiesterase (EC | 1 . . . 159 | 144/159 (90%) | |
| | 3.1.4.17) 1A, | | | |
| | calmodulin-dependent, 61K | | | |
| | brain form - bovine, 530 aa. | | | |

PFam analysis predicts that the NOV41a protein contains the domains shown in the Table 41F.

TABLE 41F
Domain Analysis of NOV41a

| | | Identities/ | |
|---|---|---|---|
| | NOV41a | Similarities | |
| Pfam | Match | for the Matched | Expect |
| Domain | Region | Region | Value |
| PDEase | 138 . . . 159 | 9/49 (18%) | 0.11 |
| | | 22/49 (45%) | |

### Example 42

The NOV42 clone was analyzed, and the nucleotide and encoded polypeptide sequences are shown in Table 42A.

TABLE 42A
NOV42 Sequence Analysis

| | SEQ ID NO: 177 | 512 bp | |
|---|---|---|---|
| NOV42a, | CCATGGCGGGCTGCGCGGCGCGGGCTCCGCCGGGCTCTGAGGCGCGTCTCAGCCTCGCCACCTTCCT | | |
| CG151822-01 | | | |
| DNA Sequence | GCTGGGCGCCTCGGTGCTCGCGCTGCCGCTGCTCACGCGCGCCGGCCTGCAGGGCCGCACCGGGCTG | | |
| | GCGCTCTACGTGGCCGGGCTCAACGCGCTGCTGCTGCTGCTCTATCGCCCGCCTCGCTACCAGATAG | | |
| | CCATCCGAGCTTGTTTCCTGGGGTTTGTGTTCGGCTGCGGCACGCTGCTAAGTTTTAGCCAGTCTTC | | |
| | TTGGAGTCACTTTGGCTGAACTGAAGCAGATTACCTGGCTCAGTGTCACACGGCTGCTGATGGTGGT | | |
| | CTTCGGAGAATGTCTGAGGAAGGCCGCCATGTNTACAGCTGGCTCCAATTTCAACCACGTGGTACAG | | |
| | AATGAAAAATCAGATACACATACTCTGGTGACCAGTGGAGTGTACGCTTGGTTTCGGCATCCTTCTT | | |
| | ACGTCGGGTGGTTTTACTGGAGTATTGGAACTCAGGTGATGCT | | |
| | ORF Start: ATG at 3 | | ORF Stop: TGA at 285 |
| | SEQ ID NO: 17 | 894 aa | MW at 9871.5 kD |
| NOV42a, | MAGCAARAPPGSEARLSLATFLLGASVLALPLLTRAGLQGRTGLALYVAGLNALLLLLYRPPRYQIA | | |
| CG151822-01 | | | |
| Protein Sequence | IRACFLGFVFGCGTLLSFSQSSWSHFG | | |
| | SEQ ID NO: 179 | 3597 bp | |
| NOV42b, | GGCACGAGCGGCGCCGCCGCCCGCTAGTCCGCCCCCCGGCGCCATGGCGGGCTGCGCGGCGCGGGCT | | |
| CG151822-02 | | | |
| DNA Sequence | CCGCCGGGCTCTGAGGCGCGTCTCAGCCTCGCCACCTTCCTGCTGGGCGCCTCGGTGCTCGCCCTGC | | |
| | CGCTGCTCACGCGCGCCGGCCTGCAGGGCCGCACCGGGCTGGCGCTCTACGTGGCCGGGCTCAACGC | | |
| | GCTGCTGCTGCTGCTCTATCGGCCGCCTCGCTACCAGATAGCCATCCGAGCTTGTTTCCTGGGGTTT | | |
| | GTGTTCGGCTGCGGCACGCTGCTAAGTTTTAGCCAGTCTTCTTGGAGTCACTTTGGCTGGTACATGT | | |
| | GCTCCCTGTCATTGTTCCACTATTCTGAATACTTGGTGACACCAGTCAATAATCCCAAAAGTCTGTC | | |
| | CTTGGATTCCTTTCTCCTGAATCACAGCCTGGAGTATACAGTAGCTGCTCTTTCTTCTTGGTTAGAG | | |
| | TTCACACTTGAAAATATCTTTTGGCCAGAACTGAAGCAGATTACCTGGCTCAGTGTCACAGGGCTGC | | |
| | TGATGGTGGTCTTCGGAGAATGTCTGAGGAAGGCCGCCATGTTTACAGCTGGCTCCAATTTCAACCA | | |
| | CGTGGTACAGAATGAAAAATCAGATACACATACTCTGGTGACCAGTGGAGTGTACGCTTGGTTTCGG | | |
| | CATCCTTCTTACGTCGGGTGGTTTTACTGGAGTATTGGAACTCAGGTGATGCTGTGTAACCCCATCT | | |
| | GCGGCGTCAGCTATGCCCTGACAGTGTGGCGATTCTTCCGCGATCGAACAGAAGAAGAAGAAATCTC | | |
| | ACTAATTCACTTTTTTGGAGAGGAGTACCTGCAGTATAAGAAGAGGGTGCCCACCGGCCTCCCTTTC | | |
| | ATAAAGGGGGTCAAGGTGGACCTGTGACGGGCAGTGGCCCCGGTGACCTTGGGGCCTCCGACCCTGT | | |
| | GCAGCCTGGGACAAAACTGTTTCCGGTTGGCCGCTCCCACATGGATTTTCTTAATCGTTTTATGTCA | | |
| | TTAGTCACTCTTCTGGAATGTCACTCAAGACCAAGCGGTCAGAAGGCCTGAGGACCCAAGGCCCCAC | | |
| | TGGAGCAGTCTGTCCTTATGCCGAATCAAGGCGGAACATGGGTGAAAGACGAGTAAGGGGCAAATCA | | |
| | CAGCAATATTCCACAGCGCCCTCCAGAGTTACCTGGGGAGGACCGAGGCCACACGCCACTGCCCCCG | | |
| | AGGCCAGAGTGTAAGTAAAGGATAACCAGGACTCGCTCGGAGAGATGGACTCTGTCCTCAGCAACAC | | |
| | TCCACAGCAGAAAGGGGTAGCAGGTACCCCTTCTTATCAGCGGTAAAAATGCATTTACAACCTTTCA | | |
| | TTTAACCGAAAAACACAGACCGCTTTAACCTCTTTATTTCTGTCCCCCACTGCATGAACATCTATAC | | |
| | AATTTTAAAAATACTTCCTCATAGGATGCTTTGGCCCTTCATCTATTTAATCATAGCTACATACCTA | | |
| | TTTTTTTATAAGTAGCAGTACACATTCAAAGGGGTATTCCTAGCTCAATGCTTGGTGTTCTAGTTCA | | |
| | ACTTTTATCCTGCAGCAAGTAAGCCTAGATAACTCTACACGATTTCGCTGAGTGGCTTTGTGTGACC | | |
| | GTGGCCCCAGGCCAAGGGGACCATGGCCCTGGCTGGCTTTCCCCCGGGGGTCTCAGCTCCTGTTGTC | | |
| | AGTGATAGGCGGCTCAAAGGAGCATCAGTTTCTTTTGATCCAAGAAGTGCTTACTGAATGCCTGCCC | | |
| | TGTGCGTGGCCTTAAACATTGAGAAGTCCTGCTCTCCGTTTATTTGGGATTTGATTCTCATTTTACC | | |
| | ATACCTTATATTCTCAATTTCAATGCCAGTCTCAGAACTCTTGTTTTCTGTGTTCTGTTCTCAAAAT | | |
| | TACATTGTCCCTCATGTCATTTCAAACTGTTTTCCAAAGGGATTTGAGCATATACAACTACAAATCC | | |
| | AAGCAGATTGACTCTCAAAAATAATCTTAAATACTGCAAATAGTCCCAACTAAGATTCAGTCAGTAT | | |
| | GTTTGTTTTGCAAGTTTCGGAGAGTAAGTTGGCTTTGAGTCACACATCGAAGCTTTAAGAGGTGAGA | | |
| | CGCTGGCTTCATTCTGGACTAGACAGGAACTTGGCCTCAGCGTGAGATCCTGCCATCCAGTGTTGCG | | |
| | GTGGCACTGAAGAAGTGTGAATGTGAAGGCGGCGTCGGCGCGGGGCCAGAGCACCACTCTGCTGCCC | | |
| | CACCACGCGGCCTGTGAGGAGCCACTAAACCTTTCCGTCCCTAGACCTCCCCATCTGTGGAATGGCG | | |
| | TCAATACCACCTACCTCACAGGGGTGTTGTGAGGACTGAGAAGAACAATGTCAAATGTTTTTAATAC | | |
| | TCAGATGTGGGAGCGACATCAATGAAATCTGTACTGTATGAAAGCTACACAAAAATGGGCAGACATT | | |
| | TGGTTAATTGTGCCAGATACCTAAAATGTATGTTCAGAAAAGCATTTTATCAACTCAGAAATATGAC | | |
| | TTATTTCTAGATTCATGGCTTAATGAATTTTTTCATTGTTATATATACCAAAGAGGCTTACGGGTTC | | |
| | ATTGATTGGTTTGAAAACCAGACAGACGGCCGGCCACGCCTGTAATCCCAAAGTGCTGGGATTGCAG | | |
| | CGTGAGCCACCACGCCCAGCCAAGATGAACTCCTTAAGGACAGGATTTGGTAAGTGATTGACTTCTT | | |
| | TTTAGTTCCATGATCTTGAGATTATTTTTAGCTTTATAAATTTAGCAGTGGCAGGGCCCGTGGAGAA | | |
| | TCAGGTTAATCAGGTAAAGCCTTTCTCGGTATTTGCTGCCAAGGCCACATCACCAATTTTCTCGATT | | |
| | TAAAAAACTGTCAAGAGATTTATTTTTCCATTGCACGTTTTAAAGTGGAGATTCTGAAGTGGAAAAT | | |
| | AGGTACTGTCAGAACAAAGCTACCTGGAAACAGCATAGAGTGAAGCCTTTCGTGAGGGCTTGCAGGC | | |
| | CGCTGCTGAGTGGCAGTTTACAGAAGAGGTCGCGGGGTGAGCCTCTTAGCAGGACAGAAAACAAGGC | | |
| | AGCAGCGCACCTGCCACCCCTTCACGAGCTGCTCCTTCAGCCTAAAAAGTAGGCTTTATTCATCCCT | | |
| | TCTGTTCATTTACCAACCTGGGGGATTGATACGACCGGGGAAAATGTTCCTAAACCAGGAAGCTGCG | | |
| | TTAGCCCATCAGGCTTTGTAAGATCTCGCCAACAGCTACCTGCTTAGGAGTACCCCCACGATACGCA | | |
| | CAGCACACCACTGTCCCTTCACTGCACTTTCTTCCTGCCTTAGGTAGTTGGGCTTGCCCACCCTAGT | | |
| | TTGCTTTTGTAGTGGTTTGGCAAGGTTAGAAGGCCTCGGCCTCTCTGTCATGCTGGGAAGTGCCTAC | | |
| | TCTCTGGGCCACTGCTGCAGAGGCCGTGGCACTTGTCATGGGTTTGGAAGACCCAGCCATCTGCAGC | | |
| | AGAGGCAGCCTATCCCATTGCAAGGAGAGGAACTGAACGGAGTAATTATTCPACTCTTCTTTTTACA | | |
| | TAAATGTTTATTTAAATATTCTAAATTGGATTTTCATTCACAGATACTGATTATTCTTTCCAGTTCT | | |
| | TAAATAAAACTGCACTTGATTTCACTCAAAAAAAAAAAAAAAAAAA | | |
| | ORF Start: ATG at 44 | | ORF Stop: TGA at 896 |
| | SEQ ID NO: 180 | 284 aa | MW at 31937.7 kD |

| | |
|---|---|
| NOV42b, | MAGCAARAPPGSEARLSLATFLLGASVLALPLLTRAGLQGRTGLALYVAGLNALLLLLYRPPRYQIA |
| CG151822-02 | |
| Protein Sequecne | IRACFLGFVFGCGTLLSFSQSSWSHFGWYMCSLSLFHYSEYLVTAVNNPKSLSLDSFLLNHSLEYTV |
| | AALSSWLEFTLENIFWPELKQITWLSVTGLLMVVFGECLRKAAMFTAGSNFNHVVQNEKSDTHTLVT |
| | SGVYAWFRHPSYVGWFYWSIGTQVMLCNPICGVSYALTVWRFERDRTEEEEISLIHFFGEEYLEYKK |
| | RVPTGLPFIKGVKVDL |

Sequence comparison of the above protein sequences yields the following sequence relationships shown in Table 42B.

TABLE 4
2B Comparison of NOV42a against NOV42b.

| | | Identities/ |
|---|---|---|
| | NOV42a Residues/ | Similarities for |
| Protein Sequence | Match Residues | the Matched Region |
| NOV42b | 1 . . . 94 | 67/94 (71%) |
| | 1 . . . 94 | 67/94 (71%) |

Further analysis of the NOV42a protein yielded the following properties shown in Table 42C.

TABLE 42C
Protein Sequence Properties NOV42a

| | |
|---|---|
| PSort | 0.6000 probability located in plasma membrane; 0.4000 |
| analysis: | probability located in Golgi body; 0.3174 probability |
| | located in mitochondrial intermembrane space; 0.3000 |
| | probability located in endoplasmic reticulum (membrane) |
| SignalP | Cleavage site between residues 37 and 38 |
| analysis: | |

A search of the NOV42a protein against the Geneseq database, a proprietary database that contains sequences published in patents and patent publication, yielded several homologous proteins shown in Table 42D.

TABLE 42D
Geneseq Results for NOV42a

| | | | Identities/ | |
|---|---|---|---|---|
| | | | Similarities for | |
| Geneseq | Protein/Organism/Length | NOV42a Residues/ | the Matched | Expect |
| Identifier | [Patent #, Date] | Match Residues | Region | Value |
| AAY32299 | Farnesyl-directed cysteine | 1 . . . 94 | 94/94 (100%) | 2e−48 |
| | carboxymethyltransferase | 1 . . . 94 | 94/94 (100%) | |
| | STE14 - Homo sapiens, 284 | | | |
| | aa. [WO9955878-A1, | | | |
| | 04 NOV. 1999] | | | |
| AAW67730 | Human prenylcysteine | 1 . . . 94 | 94/94 (100%) | 2e−48 |
| | carboxyl methyltransferase - | 1 . . . 94 | 94/94 (100%) | |
| | Homo sapiens 284 aa | | | |
| | [WO9856924-A1, | | | |
| | 17 DEC. 1998] | | | |
| AAB32052 | Human secreted protein | 12 . . . 94 | 83/83 (100%) | 3e−41 |
| | BLAST search protein SEQ | 1 . . . 83 | 83/83 (100%) | |
| | ID NO: 110 - Homo sapiens, | | | |
| | 223 aa. [WO200058350-A1, | | | |
| | 05 OCT. 2000] | | | |
| AAB32051 | Human secreted protein | 12 . . . 94 | 83/83 (100%) | 3e−41 |
| | BLAST search protein SEQ | 1 . . . 83 | 83/83 (100%) | |
| | ID NO: 109 - Homo sapiens, | | | |
| | 223 aa. [WO200058350-A1, | | | |
| | 05 OCT. 2000] | | | |
| AAY32300 | Mouse farnesyl-directed | 5 . . . 94 | 82/90 (91%) | 2e−40 |
| | cysteine | 4 . . . 93 | 83/90 (92%) | |
| | carboxymethyltransferase - | | | |
| | Mus musculus, 153 aa. | | | |
| | [WO9955878-A1, | | | |
| | 04 NOV. 1999] | | | |

In a BLAST search of public sequence datbases, the NOV42a protein was found to have homology to the proteins shown in the BLASTP data in Table 42E.

TABLE 42E
Public BLASTP Results for NOV42a

| | | | Identities/ | |
|---|---|---|---|---|
| Protein | | | Similarities for | |
| Accession | | NOV42a Residues/ | the Matched | Expect |
| Number | Protein/Organism/Length | Match Residues | Portion | Value |
| O60725 | Protein-S isoprenylcysteine | 1 . . . 94 | 94/94 (100%) | 5e−48 |
| | O-methyltransferase (EC | 1 . . . 94 | 94/94 (100%) | |
| | 2.1.1.100) (Isoprenylcysteine | | | |
| | carboxylmethyltransferase) | | | |
| | (Prenylcysteine carboxyl | | | |
| | methyltransferase) (pcCMT) | | | |
| | (Prenylated protein carboxyl | | | |
| | methyltransferase) (PPMT) - | | | |
| | Homo sapiens (Human), 284 | | | |
| | aa. | | | |
| Q9EQK7 | Protein-S isoprenylcysteine | 5 . . . 94 | 84/90 (93%) | 2e−41 |
| | O-methyltransferase (EC | 4 . . . 93 | 85/90 (94%) | |
| | 2.1.1.100) (Isoprenylcysteine | | | |
| | carboxylmethyltransferase) | | | |
| | (Prenylcysteine carboxyl | | | |
| | methyltransferase) (pcCMT) | | | |
| | (Prenylated protein carboxyl | | | |
| | methyltransferase) (PPMT) - | | | |
| | Mus musculus (Mouse), 283 | | | |
| | aa. | | | |
| O12947 | Protein-S isoprenylcysteine | 13 . . . 94 | 49/90 (54%) | 2e−19 |
| | O-methyltransferase (EC | 9 . . . 98 | 59/90 (65%) | |
| | 2.1.1.100) (Isoprenylcysteine | | | |
| | carboxylmethyltransferase) | | | |
| | (Prenylcysteine carboxyl | | | |
| | methyltransferase) (pcCMT) | | | |
| | (Prenylated protein carboxyl | | | |
| | methyltransferase) (PPMT) | | | |
| | (Farnesyl cysteine carboxyl | | | |
| | methyltransferase) (FCMT) - | | | |
| | Xenopus laevis (African | | | |
| | clawed frog), 288 aa. | | | |
| Q9WVM4 | Protein-S isoprenylcysteine | 53 . . . 94 | 39/42 (92%) | 8e−17 |
| | O-methyltransferase (EC | 1 . . . 42 | 40/42 (94%) | |
| | 2.1.1.100) (Isoprenylcysteine | | | |
| | carboxylmethyltransferase) | | | |
| | (Prenylcysteine carboxyl | | | |
| | methyltransferase) (pcCMT) | | | |
| | (Prenylated protein carboxyl | | | |
| | methyltransferase) (PPMT) | | | |
| | (Farnesyl cysteine carboxyl | | | |
| | methyltransferase) (FCMT) - | | | |
| | Rattus norvegicus (Rat), 232 | | | |
| | aa (fragment). | | | |
| Q9R1L8 | Farnesyl cysteine carboxyl | 65 . . . 94 | 28/30 (93%) | 4e−10 |
| | methyltransferase - Rattus | 1 . . . 30 | 29/30 (96%) | |
| | norvegicus (Rat), 33 aa | | | |
| | (fragment). | | | |

### Example 43

The NOV43 clone was analyzed, and the nucleotide and encoded polypeptide sequences are shown in Table 43A.

TABLE 43A
NOV43 Sequence Analysis

| | SEQ ID NO: 181 | 2306 bp | |
|---|---|---|---|
| NOV43a, | GCCATGGCGTCCTGCGTGGGGAGCCGGACCCTAAGCAAGGATCATGTGAACTACAAAATGCATTTCC | | |
| CG152256-01 | | | |
| DNA Sequence | CGATGATCAACGAGCAGCAAGTGGAGGACATCACCATTGACTTCTTCTACCGGCCGCATACCATCAC | | |
| | GACAACATCTGGAGAGGCATCCTCTCTGTTATTTTCTTCTTTCTTATCATCAGTGTGTTAGCTTTCC | | |
| | CTCTTCTTCATGCATCTCCTCCCCAATTTTCCCGAGTGCTGGTGGGATCAAGTCATTCTGGACATCC | | |
| | TGTTGTGCAATGGCGGTGGCATTTGGCTGGGCATGGTCGTTTGCCGGTTTTTAGAGATGAGGACTTA | | |
| | CCACTGGGCAAGCTTCAAGGACATTCATACCACCACCGGGAAGATCAAGAGAGCTGTTCTGCAGTTC | | |
| | ACTCCTGCTAGCTGGACCTATGTTCGATGGTTTGACCCCAAATCTTCTTTTCAGAGAGTAGCTGGAG | | |
| | TGTACCTTTTCATGATCATCTGGCAGCTGACTGAGTTGAATACCTTCTTCTTGAAGCATATCTTTGT | | |
| | GTTCCAAGCCAGTCATCCATTAAGTTCGGGTAGAATTCTCTTTATTGGTGGCATCACAGCTCCCACA | | |
| | GTGAGACAGTACTACGCTTACCTCACCGACACACAGTGCAACCGCGTAGGAACACAATGCTGGGTGT | | |
| | TTCGGGCTTTCACCACTTTCCTCTCTCTGTACGGCATGATTTGGTATGCAGAACACTATGGTCACCG | | |
| | AGAAAAGACCTACTCGGAGTGTGAAGATGGCACCTACAGTCCAGAGATCTCCTGGCATCACAGGAAA | | |
| | CGGACAAAAGGTTCTGAAGACAGCCCACCCAAGCATGCAGGCAACAACGAAAGCCATTCTTCCAGGA | | |
| | GAAGGAATCGGCATTCCAAGTCAAAAGTCACCAATGGCGTTGGAAAGAAATGAAAAACCCTGGTTAA | | |
| | TCAAAGATGTTCCAGAGTGCCTAGAACTCAGAGGGAAATGGAACTCATTTGGAACTCCCCGTGAGGA | | |
| | GGTCGAGGCGCACAGGGCAAGCAGCAAGAGGCGAGGGCACTTGGGGGTCATTATTTGAGATCGTAAG | | |
| | TCTTGTTTCCCACAGACCTGGCCGCGTCAGGCAGATCATCGCCTGGGCGGCCTTTGCCAACGTGGGG | | |
| | TCTCTTCTAACTTCAGCACTTGACATGCGGTCACCGGTGCCAGCGCGGTGTGTTGAAGGGAAACGGT | | |
| | AGCTATTCATTCACAGTTGCCAAGAGCACCTCCGCGCCTGCTGGATCGTGGATGCAGCGTAAACATC | | |
| | TTCCTTCAGACGAGGCATTAACCCCATGGTTAATGGACTGGTCACCAGTTTTTATTTTATTTTTATG | | |
| | AATCTACCTTTCCATTGATTGATTTAAGTTCAGGCCACTTTTCTGTCTTTTATTTGGTTACTGTTGT | | |
| | TATTTGTTTTTAAGTTAGGATGCTTTTTAACAGCCTTTAGAAGCCGCTGCTGAAATTGATACTGGGG | | |
| | GAAGGGTTCCCCTTCCTTCTAGAGCAGAAAAGGGAGAGAAGTGTTGTATTCCTGTTTGGTAACCTCA | | |
| | GTCTCCTGTAAGACCTCCTACCACATGGCGAGTATACACCAATCAGGAGAGGGTAGCTGCCTGCATA | | |
| | GGAGCCTCGCTTCCGATTATTCCCTTCCCAATATTATTCATCCAGACTTACCCACAGTGCACAAAAG | | |
| | CAAACCTGCTAGAGAGGCAGTGAACACCACAGCTTCTCCCCAGCTTGGTGCCTTTTACATCGGGTTT | | |
| | GTTCTCCTTCCATGGTGTGTTGCTGACATTGTCACTGAGTCCCATGTGACGTGCTGGTGAGTATTAC | | |
| | CTTTCATCTGTGCCATGCTCTAGAACCTTGACCTTGATAGTTCACCACGTCTGATGGATCCCTGTTT | | |
| | TAAATAAAAACGATTCACTTTAAAGCCT | | |
| | ORF Start: ATG at 4 | | ORF Stop: TGA at 1324 |
| | SEQ ID NO: 182 | 440 aa | MW at 51772.5 kD |

| | |
|---|---|
| NOV43a, | MASCVGSRTLSKDDVNYKMHFRMTNEQQVEDITIDFFYRPHTITLLSPTTVSLMYFAFTRDDSVPED |
| CG152256-01 | |
| Protein Sequence | NIWRGILSVIFFFLIISVLAFPNGPFTRPHPALWRMVFGLSVLYFLFLVFLLFLNFEQVKSLMYWLD |
| | PNLRYATREADVMEYAVNCHVITWERIISHFDIFAFGHFWGWAMKALLIRSYGLCWTISITWELTEL |
| | FFMHLLPNFAECWWDQVILDILLCNGGGIWLGMVVCRFLEMRTYHWASFKDIHTTTGKIKRAVLQFT |
| | PASWTYVRWFDPKSSFQRVAGVYLFMIIWQLTELNTFFLKHIFVFQASHPLSWGRILFIGGITAPTV |
| | RQYYAYLTDTQCKRVGTQCWVFGAFTTFLCLYGMTWYAEHYGNREKTYSECEDGTYSPEISWHHRKG |
| | TKGSEDSPPKHAGNNESHSSRRRNRHSKSKVTNGVGKK |

Further analysis of the NOV43a protein yielded the following properties shown in Table 43B.

TABLE 43B
Protein Sequence Properties NOV43a

| | |
|---|---|
| PSort analysis: | 0.6000 probability located in plasma membrane; |
| | 0.4000 probability located in Golgi body; |
| | 0.3000 probability located in endoplasmic |
| | reticulum (membrane); 0.0300 probability |
| | located in mitochondrial inner membrane |
| SignalP analysis: | No Known Signal Sequence Predicted |

A search of the NOV43a protein against the Geneseq database, a proprietary database that contains sequences published in patents and patent publication, yielded several homologous proteins shown in Table 43C.

TABLE 43C
Geneseq Results for NOV43a

| | | | Identities/ | |
|---|---|---|---|---|
| | | | Similarities | |
| Geneseq | Protein/Organism/Length | NOV43a Residues/ | for the | Expect |
| Identifier | [Patent #, Date] | Match Residues | Matched Region | Value |
| ABB89640 | Human polypeptide | 1 . . . 440 | 440/473 (93%) | 0.0 |
| | SEQ ID NO 2016 - | 1 . . . 473 | 440/473 (93%) | |
| | Homo sapiens, 473 | | | |
| | aa. [WO200190304-A2, | | | |
| | 29 NOV. 2001] | | | |
| AAB58945 | Breast and ovarian cancer | 1 . . . 440 | 439/473 (92%) | 0.0 |
| | associated antigen protein | 44 . . . 516 | 439/473 (92%) | |
| | sequence SEQ ID 653 - | | | |
| | Homo sapiens, 516 aa. | | | |
| | [WO200055173-A1, | | | |
| | 21 SEP. 2000] | | | |
| ABB71324 | Drosophila melanogaster | 3 . . . 359 | 206/357 (57%) | e−133 |
| | polypeptide SEQ ID NO | 59 . . . 412 | 276/357 (76%) | |
| | 40764 - Drosophila | | | |
| | melanogaster, 498 aa. | | | |
| | [WO200171042-A2, | | | |
| | 27 SEP. 2001] | | | |
| AAB73515 | Human transferase HTFS-22, | 22 . . . 361 | 128/351 (36%) | 2e−60 |
| | SEQ ID NO: 22 - Homo | 45 . . . 389 | 185/351 (52%) | |
| | sapiens, 487 aa. | | | |
| | [WO200132888-A2, | | | |
| | 10 MAY 2001] | | | |
| AAM79907 | Human protein SEQ ID NO | 22 . . . 361 | 128/351 (36%) | 2e−60 |
| | 3553 - Homo sapiens, 529 aa. | 63 . . . 407 | 185/351 (52%) | |
| | [WO200157190-A2, | | | |
| | 09 AUG. 2001] | | | |

In a BLAST search of public sequence datbases, the NOV43a protein was found to have homology to the proteins shown in the BLASTP data in Table 43D.

TABLE 43D
Public BLASTP Results for NOV43a

| | | | Identities/ | |
|---|---|---|---|---|
| Protein | | | Similarities for | |
| Accession | | NOV43a Residues/ | the Matched | Expect |
| Number | Protein/Organism/Length | Match Residues | Portion | Value |
| P48651 | Phosphatidylserine synthase I | 1 . . . 440 | 440/473 (93%) | 0.0 |
| | (Serine-exchange enzyme I) | 1 . . . 473 | 440/473 (93%) | |
| | (EC 2.7.8.-) - Homo sapiens | | | |
| | (Human), 473 aa. | | | |
| Q99LH2 | Similar to phosphatidylserine | 1 . . . 440 | 428/473 (90%) | 0.0 |
| | synthase 1 - Mus musculus | 1 . . . 473 | 437/473 (91%) | |
| | (Mouse), 473 aa. | | | |
| Q00576 | Phosphatidylserine synthase I | 1 . . . 440 | 428/473 (90%) | 0.0 |
| | (Serine-exchange enzyme I) | 1 . . . 471 | 434/473 (91%) | |
| | (EC 2.7.8.-) - Cricetulus | | | |
| | longicaudatus (Long-tailed | | | |
| | hamster) (Chinese hamster), | | | |
| | 471 aa. | | | |
| O55024 | Phosphatidylserine synthase- | 1 . . . 440 | 421/473 (89%) | 0.0 |
| | 1 - Mus musculus (Mouse), 473 | 1 . . . 473 | 432/473 (91%) | |
| | aa. | | | |
| Q9BSY0 | Similar to phosphatidylserine | 145 . . . 440 | 292/329 (88%) | e−178 |
| | synthase 1 - Homo sapiens | 6 . . . 334 | 293/329 (88%) | |
| | (Human), 334 aa (fragment). | | | |

PFam analysis predicts that the NOV43a protein contains the domains shown in the Table 43E.

TABLE 43E
Domain Analysis of NOV43a

| | | Identities/ | |
|---|---|---|---|
| | NOV43a | Similarities | |
| | Match | for the | |
| Pfam Domain | Region | Matched Region | Expect Value |
| COLFI | 119 . . . 137 | 10/19 (53%) | 0.12 |
| | | 14/19 (74%) | |
| PSS | 96 . . . 370 | 179/310 (58%) | 1.1e−206 |
| | | 267/310 (86%) | |

### Example 44

The NOV44 clone was analyzed, and the nucleotide and encoded polypeptide sequences are shown in Table 44A.

TABLE 44A
NOV44 Sequence Analysis

| | SEQ ID NO: 183 | 1151 bp | |
|---|---|---|---|
| NOV44a, | CNTGNATTTGGCCGCGGGGCCATGTAGCTCCGAGCGGCGGATCGCGAGCCTCCTGCGAACCCCAGCC | | |
| CG171804-01 | | | |
| DNA Sequence | TGCACGCCCGGTTAGCATTCCGCCGGGAGATGCGGCAGTGGAATCTGGAAGGGCGGTGAAAAACCTA | | |
| | CGTCCTGCCCTCGCCCGGCCTCTCCATTCGTCCCCCGGGTAGAGAGGGTCGGCTCGTGCTCATCATC | | |
| | CTGTGCTCCGTGGTCTTCTCTGCCCTCTACATCCTCCTGTGCTGCTGGGCCGGCCTGCCCCTCTGCC | | |
| | TGGCCACCTGCCTGGACCACCACTTCCCCACAGGCTCCAGGCCCACTGTGCCGGGACCCCTGCACTT | | |
| | CAGTGGATATAGCAGTGTGCCAGATGGCAAGCCGCTGGTCCGCGAGCCCTGCCGCAGCTGTGCCGTG | | |
| | GTGTCCAGCTCCGGCCAAATGCTGGGCTCAGGCCTGGGTGCTGAGATCGACAGTGCCGAGTGCGTGT | | |
| | TCCGCATGAACCAGGCGCCCACCGTGGGCTTTGAGGCGGATGTCGGCCAGCGCAGCACCCTGCGTGT | | |
| | CGTCTCACACACAAGCGTGCCGCTGCTGCTGCGCAACTATTCACACTACTTCCAGAAGGCCCGAGAC | | |
| | ACGCTCTACATGGTGTGGGGCCAGGGCAGGCACATGGACCGGGTGCTCGGCGGCCGCACCTACCGCA | | |
| | CGCTGCTGCAGCTCACCAGGATGTACCCCGGCCTGCAGGTGTACACCTTCACGGAGCGCATGATGGC | | |
| | CTACTGCGACCAGATCTTCCAGGACGAGACGGGCAAGAACCGGAGGCAGTCGGGCTCCTTCCTCAGC | | |
| | ACCGGCTGGTTCACCATGATCCTCGCGCTGGAGCTGTGTGAGGAGATCGTGGTCTATGGGATGGTCA | | |
| | GCGACAGCTACTGCAGGGAGAAGAGCCACCCCTCAGTGCCTTACCACTACTTTGAGAAGGGCCGGCT | | |
| | AGATGAGTGTCAGATGTACCTGGCACACGAGCAGGCGCCCCGAACCCCCCACCGCTTCATCACTGAG | | |
| | AAGGCGGTCTTCTCCCGCTGGGCCAAGAAGAGGCCCATCGTGTTCGCCCATCCGTCCTGGAGGACTG | | |
| | AGTAGCTTCCGTCGTCCTGCCAGCCGCCATGCCGTTCCGAGGCCTCCGGGATGTCCCATCCCAAGCC | | |
| | ATCACACTCCAC | | |
| | ORF Start: ATG at 421 | | ORF Stop: TAG at 1075 |
| | SEQ ID NO: 184 | 218 aa | MW at 25333.8 kD |

| | |
|---|---|
| NOV44a, | MLGSGLGAEIDSAECVFRMNQAPTVGFEADVGQRSTLRVVSHTSVPLLLRNYSHYFQKARDTLYMVW |
| CG171804-01 | |
| Protein Sequence | GQGRHMDRVLGGRTYRTLLQLTRMYPGLQVYTFTERMMAYCDQIFQDETGKNRRQSGSFLSTGWFTM |
| | ILALELCEEIVVYGMVSDSYCREKSEPSVPYHYFEKGRLDECQMYLAHEQAPRSAHRFITEKAVFSR |
| | WAKKRPIVFAHPSWRTE |

Further analysis of the NOV44a protein yielded the following properties shown in Table 44B.

TABLE 44B
Protein Sequence Properties NOV44a

| | |
|---|---|
| PSort analysis: | 0.6400 probability located in microbody (peroxisome); |
| | 0.3000 probability located in nucleus; 0.2068 |
| | probability located in lysosome (lumen); 0.1000 |
| | probability located in mitochondrial matrix space |
| SignalP analysis: | No Known Signal Sequence Predicted |

A search of the NOV44a protein against the Geneseq database, a proprietary database that contains sequences published in patents and patent publication, yielded several homologous proteins shown in Table 44C.

TABLE 44C
Geneseq Results for NOV44a

| | | | Identities/ | |
|---|---|---|---|---|
| | | | Similarities | |
| Geneseq | Protein/Organism/Length | NOV44a Residues/ | for the | Expect |
| Identifier | [Patent #, Date] | Match Residues | Matched Region | Value |
| AAB75350 | Human secreted protein #9 - | 1 . . . 218 | 218/218 (100%) | e−128 |
| | Homo sapiens, 302 aa. | 85 . . . 302 | 218/218 (100%) | |
| | [WO200100806-A2, | | | |
| | 04 JAN. 2001] | | | |
| AAB61614 | Human protein HP03380 - | 1 . . . 218 | 218/218 (100%) | e−128 |
| | Homo sapiens, 302 aa. | 85 . . . 302 | 218/218 (100%) | |
| | [WO200102563-A2, | | | |
| | 11 JAN. 2001] | | | |
| AAB25764 | Human secreted protein SEQ | 1 . . . 218 | 218/218 (100%) | e−128 |
| | ID #76 - Homo sapiens, 302 | 85 . . . 302 | 218/218 (100%) | |
| | aa. [WO200037491-A2, | | | |
| | 29 JUN. 2000] | | | |
| AAB28674 | Human | 1 . . . 218 | 218/218 (100%) | e−128 |
| | carbohydrate-modifying | 85 . . . 302 | 218/218 (100%) | |
| | enzyme Incyte ID No: | | | |
| | 983984CD1 - Homo sapiens, | | | |
| | 302 aa. [WO200063351-A2, | | | |
| | 26 OCT. 2000] | | | |
| AAB24495 | Human secreted protein | 1 . . . 218 | 217/218 (99%) | e−128 |
| | sequence encoded by gene 5 | 128 . . . 345 | 217/218 (99%) | |
| | SEQ ID NO: 120 - Homo | | | |
| | sapiens, 345 aa. | | | |
| | [WO200035937-A1, | | | |
| | 22 JUN. 2000] | | | |

In a BLAST search of public sequence datbases, the NOV44a protein was found to have homology to the proteins shown in the BLASTP data in Table 44D.

TABLE 44D
Public BLASTP Results for NOV44a

| | | | Identities/ | |
|---|---|---|---|---|
| Protein | | | Similarities for | |
| Accession | | NOV44a Residues/ | the Matched | Expect |
| Number | Protein/Organism/Length | Match Residues | Portion | Value |
| Q9H4F1 | Alpha-N-acetyl-neuraminyl-2,3-beta- | 1 . . . 218 | 218/218 (100%) | e−128 |
| | galactosyl-1,3-N-acetylgalactosaminide | 85 . . . 302 | 218/218 (100%) | |
| | alpha-2,6-sialyltransferase (EC 2.4.99.7) | | | |
| | (NeuAc-alpha-2,3-Gal-beta-1,3-GalNAc- | | | |
| | alpha-2, 6-sialyltransferase) (ST6GalNAc | | | |
| | IV) (Sialyltransferase 7D) - Homo sapiens | | | |
| | (Human), 302 aa. | | | |
| Q9H4F1 | Alpha2,6-sialyltransferase - Homo sapiens | 1 . . . 218 | 217/218 (99%) | e−128 |
| | (Human), 302 aa. | 85 . . . 302 | 218/218 (99%) | |
| Q9NWU6 | CDNA FLJ20593 fis, clone KAT08984 - | 1 . . . 218 | 217/218 (99%) | e−127 |
| | Homo sapiens (Human), 302 aa. | 85 . . . 302 | 217/218 (99%) | |
| Q9UKU1 | NeuAc-alpha-2,3-Gal-beta-1,3-GalNAc- | 1 . . . 218 | 216/218 (99%) | e−127 |
| | alpha-2, 6-sialyltransferase | 85 . . . 302 | 216/218 (99%) | |
| | alpha2,6-sialyltransferase - Homo sapiens | | | |
| | (Human), 302 aa. | | | |
| Q9R2B6 | Alpha-N-acetyl-neuraminyl-2,3-beta- | 1 . . . 218 | 202/218 (92%) | e−118 |
| | galactosyl-1,3-N-acetylgalactosaminide | 143 . . . 360 | 207/218 (94%) | |
| | alpha-2,6-sialyltransferase (EC 2.4.99.7) | | | |
| | (NeuAc-alpha-2,3-Gal-beta-1,3-GalNAc- | | | |
| | alpha-2, 6-sialyltransferase) (ST6GalNAc | | | |
| | IV) (Sialyltransferase 7D) - Mus musculus | | | |
| | (Mouse), 360 aa. | | | |

PFam analysis predicts that the NOV44a protein contains the domains shown in the Table 44E.

TABLE 44E
Domain Analysis of NOV44a

| | | Identities/ | |
|---|---|---|---|
| | NOV44a | Similarities | |
| | Match | for the Matched | Expect |
| Pfam Domain | Region | Region | Value |
| Glyco_transf_29 | 1 . . . 202 | 65/324 (20%) | 6e−43 |
| | | 184/324 (57%) | |

### Example 45

The NOV45 clone was analyzed, and the nucleotide and encoded polypeptide sequences are shown in Table 45A.

TABLE 45A
NOV45 Sequence Analysis

| | SEQ ID NO: 185 | 1475 bp | |
|---|---|---|---|
| NOV45a, | AGGACTCCAAGCGCCATGGCCGCTGCCCCCCGAGCCCGGGTCGCGTACTTGCTCAGGCAACTCCAAC | | |
| CG171841-01 | | | |
| DNA Sequence | GCGCAGCATGGCTGTTTCAAATATTAGATATGGAGCAGCAGTTACAAAGGAAGTAGGAATGGCAGAC | | |
| | CTAAAAAACATGGGTGCTAAAAATGTGTGCTTGATGACAGACAAGAACCTCTCCAAGCTCCCTCCTG | | |
| | TGCAAGTAGCTATGGATTCCCTAGTGAAGAATGGCATCCCCTTTACGGTTTATGATAATGTGAGAGT | | |
| | GGAACCAACGGATAGCTTCATGGAAGCTATTGAGTTTGCCCAAAAGGGAGCTTTTGATGCCTATGTT | | |
| | CCTGTCGGTGGTGGCTCTACCATGGACACCTGTAAGGCTGCTAATCTGTATGCATCCAGCCCTCATT | | |
| | CTGATTTCCTAGATTATGTCAGTGCCCCCATTGGCAAGGGAAAGCCTGTGTCTGTGCCTCTTAAGCC | | |
| | TCTGATTGCAGTGCCAACTACCTCAGGAACCGGGAGTGAAACTACTGGGGTTGCCATTTTTGACTAT | | |
| | GAACACTTGAAAGTAAAAATTGGCATCACTTCGAGAGCCATCAAACCCACACTGGGACTGATTGATC | | |
| | CTCTGCACACCCTCCACATGCCTGCCCGAGTGGTCGCCAACAGTGGCTTTCATGTGTTTAGCCATGC | | |
| | CCTGGAGTCATACACCACCCTGCCCTACCACCTGCGGAGCCCCTGCCCTTCAAATCCCATCACACGG | | |
| | CCTGCGTACCAGGGCAGCAACCCAATCAGTGACATTTGGGCTATCCACGCGCTGCGGATCGTGGCTA | | |
| | AGTATCTGAAGGCTGTCAGAAATCCCGATGATCTTGAAGCAAGGTCTCATATGCACTTGGCAAGTGC | | |
| | TTTTGCTGGCATCGGCTTTGGAAATGCTCCTGTTCATCTGCATGGAATGTCTTACCCAATTTCAGGT | | |
| | TTAGTGAAGATGTATAAAGCAAAGCATTACAATGTGGATCACCCACTGGTCCCCCATGGCCTTTCTG | | |
| | TGGTGCTCACGTCCCCAGCGGTGTTCACTTTCACCGCCCAGATGTTTCCAGAGCGACACCTGGAGAT | | |
| | GGCACAACTTCTAGGAGCCGACACCCGCACTGCCAGGATCCAAGATCCAGGGCTGGTGTTGGCAGAC | | |
| | ACGCTCCGGAAATTCTTATTCGATCTGGATGTTGATGATGGCCTAGCAGCTGTTGGTTACTCCAAAG | | |
| | CTGATATCCCCGCACTAGTGAAACGAACGCTGCCCCAGGAAAGGGTCACCAAGCTTGCACCCTGTCC | | |
| | CCAGTCAGAAGAGGATCTGGCTGCTCTGTTTGAAGCTTCAATGAAACTGTATTAATTGTCATTTTAA | | |
| | CTGAAAGAATTACCGCTGGCCATTGTAGTGCTGAGAGCAAGAGCTGATCTAGCTAGGGCTTTGTCTT | | |
| | TTCATCTTTGCGCATAACTTACCTGTTACCAGTATAGGTGGGATATACATTTATCTTCCAGCAAATT | | |
| | C | | |
| | ORF Start: ATG at 75 | | ORF Stop: TAA at 1326 |
| | SEQ ID NO: 186 | 417 aa | MW at 44871.2 kD |

| | |
|---|---|
| NOV45a, | MAVSNIRYGAAVTKEVCMADLKNMGAKNVCLNTDKNLSKLPPVQVAMDSLVKNGIPFTVYDNVRVEP |
| CG171841-01 | |
| Protein Sequence | TDSFMEAIEFAQKGAFDAYVAVGGGSTMDTCKAANLYASSPHSDFLDYVSAPIGKGKPVSVPLKPLI |
| | AVPTTSGTGSETTGVAIFDYEHLKVKIGITSRAIKPTLGLIDPLHTLHMPARVVANSGFDVFSHALE |
| | SYTTLPYHLRSPCPSNPITRPAYQGSNPISDIWAIHALRIVAKYLKAVRNPDDLEARSHMHLASAFA |
| | GIGFGNAGVHLHGMSYPISGLVKMYKAKDYNVDHPLVPHGLSVVLTSPAVFTFTAQMFPERHLEMAE |
| | LLGADTRTARIQDAGLVLADTLRKFLFDLDVDDGLAAVGYSKADIPALVKGTLPQERVTKLAPCPQS |
| | EEDLAALFEASMXLY |

Further analysis of the NOV45a protein yielded the following properties shown in Table 45B.

TABLE 45B
Protein Sequence Properties NOV45a

| | |
|---|---|
| PSort analysis: | 0.4500 probability located in cytoplasm; 0.3188 |
| | probability located in microbody (peroxisome); |
| | 0.2355 probability located in lysosome (lumen); |
| | 0.1000 probability located in mitochondrial |
| | matrix space |
| SignalP analysis: | No Known Signal Sequence Predicted |

A search of the NOV45a protein against the Geneseq database, a proprietary database that contains sequences published in patents and patent publication, yielded several homologous proteins shown in Table 45C.

TABLE 45C
Geneseq Results for NOV45a

| | | | Identities/ | |
|---|---|---|---|---|
| | | | Similarities for | |
| Geneseq | Protein/Organism/Length | NOV45a Residues/ | the Matched | Expect |
| Identifier | [Patent #, Date] | Match Residues | Region | Value |
| AAE21522 | Human dehydrogenase | 1 . . . 417 | 413/420 (98%) | 0.0 |
| | DHDR-6 protein - Homo | 49 . . . 467 | 414/420 (98%) | |
| | sapiens, 467 aa. | | | |
| | [WO200216562-A2, | | | |
| | 28 FEB. 2002] | | | |
| AAB73686 | Human oxidoreductase | 1 . . . 417 | 412/420 (98%) | 0.0 |
| | protein ORP-19 - Homo | 49 . . . 467 | 413/420 (98%) | |
| | sapiens, 467 aa. | | | |
| | [WO200144448-A2, | | | |
| | 21 JUN. 2001] | | | |
| ABB59876 | Drosophila melanogaster | 1 . . . 417 | 254/420 (60%) | e−146 |
| | polypeptide SEQ ID NO | 46 . . . 464 | 327/420 (77%) | |
| | 6420 - Drosophila | | | |
| | melanogaster, 464 aa. | | | |
| | [WO200171042-A2, | | | |
| | 27 SEP. 2001] | | | |
| ABG08093 | Novel human diagnostic | 62 . . . 322 | 240/268 (89%) | e−131 |
| | protein #8084 - Homo | 1 . . . 268 | 243/268 (90%) | |
| | sapiens, 268 aa. | | | |
| | [WO200175067-A2, | | | |
| | 11 OCT. 2001] | | | |
| AAB42855 | Human ORFX ORF2619 | 247 . . . 417 | 168/172 (97%) | 7e−91 |
| | polypeptide sequence SEQ | 41 . . . 212 | 170/172 (98%) | |
| | ID NO: 5238 - Homo sapiens, | | | |
| | 212 aa. [WO200058473-A2, | | | |
| | 05 OCT. 2000] | | | |

In a BLAST search of public sequence datbases, the NOV45a protein was found to have homology to the proteins shown in the BLASTP data in Table 45D.

TABLE 45D
Public BLASTP Results for NOV45a

| | | | Identities/ | |
|---|---|---|---|---|
| Protein | | | Similarities for | |
| Accession | | NOV45a Residues/ | the Matched | Expect |
| Number | Protein/Organism/Length | Match Residues | Portion | Value |
| CAD28993 | Sequence 4 from Patent | 1 . . . 417 | 413/420 (98%) | 0.0 |
| | WO0216562 - Homo sapiens | 49 . . . 467 | 414/420 (98%) | |
| | (Human), 467 aa. | | | |
| Q96MF9 | CDNA FLJ32430 fis, clone | 1 . . . 417 | 412/420 (98%) | 0.0 |
| | SKMUS2001129, weakly | 1 . . . 419 | 413/420 (98%) | |
| | similar to NAD-dependent | | | |
| | methanol dehydrogenase (EC | | | |
| | 1.1.1.244) - Homo sapiens | | | |
| | (Human), 419 aa. | | | |
| Q8RON6 | Hypothetical 45.0 kDa | 1 . . . 417 | 372/420 (88%) | 0.0 |
| | protein - Mus musculus | 1 . . . 419 | 394/420 (93%) | |
| | (Mouse), 419 aa. | | | |
| Q9W265 | T3DH protein - Drosophila | 1 . . . 417 | 254/420 (60%) | e−145 |
| | melanogaster (Fruit fly), 464 | 46 . . . 464 | 327/420 (77%) | |
| | aa. | | | |
| Q95S86 | GM05887p - Drosophila | 1 . . . 417 | 254/420 (60%) | e−145 |
| | melanogaster (Fruit fly), 425 | 7 . . . 425 | 327/420 (77%) | |
| | aa. | | | |

PFam analysis predicts that the NOV45a protein contains the domains shown in the Table 45E.

TABLE 45E
Domain Analysis of NOV45a

| Pfam Domain | NOV45a | Identities/ | Expect |
|---|---|---|---|
| | Match | Similarities | Value |
| | Region | for the | |
| | | Matched Region | |
| Fe-ADH | 4 . . . 205 | 68/216 (31%) | 5.6e−28 |
| | | 143/216 (66%) | |
| Fe-ADH | 228 . . . 288 | 30/68 (44%) | 2.5e−10 |
| | | 51/68 (75%) | |

### Example 46

The NOV46 clone was analyzed, and the nucleotide and encoded polypeptide sequences are shown in Table 46A.

TABLE 46A
NOV46 Sequence Analysis

| | SEQ ID NO: 187 | 1310 bp | |
|---|---|---|---|
| NOV46a, | CTACTCTCAGCCAGGAATCATGTCTTGGGCCGCTCGCCCGCCCTTCCTCCCTCAGCGGCATGCCGCA | | |
| CG173017-01 | | | |
| DNA Sequence | GGGCAGTGTGGGCCGGTGGGGGTGCGAAAAGAAATGCATTGTGGGGTCGCGTCCCGGTGGCGGCGGC | | |
| | GACGGCCCTGGCTGGATCCCGCAGCGGCGGCGGCGGCGGCGGTGGCAGGCGGAGAACAACAACCCC | | |
| | GGAGCCGGAGCCAGGGGAGGCTGGACGCGACGGGATGGGCGACAGCGGGCGGGTGGCCCTGGGGCT | | |
| | GGCAAACGCCTATGTGCAATCTGCGGGGACAGAAGCTCACGCAAACACTACGGGGTTTACAGCTGTG | | |
| | AGGGTTGCAAGGGCTTCTTCAAACGCACCATCCGCAAAGACCTTACATACTCTTGCCGGGACAACAA | | |
| | AGACTGCACAGTGGACAAGCGCCAGCGGAACCGCTGTCAGTACTGCCGCTATCAGAAGTGCCTGGCC | | |
| | ACTGGCATGAAGAGGGAGGCGGTACAGGAGGAGCGTCAGCGGGGAAAGGACAGGGATGGGGATGGGG | | |
| | AGGGGGCTGGGGGAGCCCCCGAGGAGATGCCTGTGGACAGGATCCTGGAGGCAGAGCTTGCTGTGGA | | |
| | ACAGAAGAGTGACCAGGGCGTTGAGGGTCCTGGGGGAACCGGGGGTAGCGGCAGCAGCCCAAATGAC | | |
| | CCTGTGACTAACATCTGTCAGGCAGCTGACAAACAGCTATTCACGCTTGTTGAGTGGGCGAAGAGGA | | |
| | TCCCACACTTTTCCTCCTTGCCTCTGGATGATCAGGTCATATTGCTGCGGGCAGGCTGGAATGAACT | | |
| | CCTCATTGCCTCCTTTTCACACCGATCCATTGATGTTCGAGATGGCATCCTCCTTGCCACAGGTCTT | | |
| | CACGTGCACCGCAACTCAGCCCATTCAGCAGGAGTAGGAGCCATCTTTGATCGGGTGCTGACAGAGC | | |
| | TAGTGTCCAAAATCCGTGACATGAGGATGGACAAGACAGAGCTTGGCTGCCTGAGGGCAATCATTCT | | |
| | GTTTAATCCAGATGCCAAGGGCCTCTCCAACCCTAGTGAAGGTGGAGGTCCTGCGGGAGAAGTGTAT | | |
| | GCATCACTGGAGACCTACTGCAAACAGAAGTACCCTGAGCAGCAGGGACGGTTTCCCAAGCTGCTGC | | |
| | TACGTCTTCCTGCCCTCCGGTCCATTCGCCTTAAGTGTCTAGAGCATCTGTTTTTCTTCAAGCTCAT | | |
| | TGGTGACACCCCCATCGACACCTTCCTCATGGAGATGCTTGAGGCTCCCCATCAACTGGCCTGAGCT | | |
| | CAGACCCAGACGTGGTGCTTCTCCACACTGGAGGAGC | | |
| | ORF Start: ATG at 20 | | ORF stop: TGA at 1268 |
| | SEQ ID NO: 188 | 416 aa | MW at 45778.7 kD |

| | |
|---|---|
| NOV46a, | MSWAARPPFLPQRHAAGQCGPVGVRKEMHCGVASRWRRRRPWLDPAAAAAAAVACGEQQTPEPEPGE |
| CG173017-01 | |
| Protein Sequence | AGRDGMGDSGRGGPGAGKRLCAICGDRSSGKHYGVYSCEGCKGFFKRTIRKDLTYSCRDNKDCTVDK |
| | RQRNRCQYCRYQKCLATGMKREAVQEERQRGKDRDGDGEGAGGAPEEMPVDRILEAELAVEQKSDQG |
| | VEGPGGTGGSGSSPNDPVTNTCQAADKQLFTLVEWAKRIPHFSSLPLDDQVILLRAGWNELLIASFS |
| | HRSIDVRDGILLATGLHHRNSAflSAGVGAIFDRVLTELVSKMRDMRMDKTELGCLRAITLFNPDAK |
| | GLSNPSEVEVLREKVYASLETYCKQKYPEQQGRFAXLLLRLPALRSIGLKCLEHLFFFKLIGDTPID |
| | TFLMEMLEAPHQLA |

Further analysis of the NOV46a protein yielded the following properties shown in Table 46B.

TABLE 46B
Protein Sequence Properties NOV46a

| | |
|---|---|
| PSort analysis: | 0.9700 probability located in nucleus; 0.3000 |
| | probability located in microbody (peroxisome); |
| | 0.1000 probability located in mitochondrial matrix |
| | space; 0.1000 probability located in lysosome (lumen) |
| SignalP analysis: | No Known Signal Sequence Predicted |

A search of the NOV46a protein against the Geneseq database, a proprietary database that contains sequences published in patents and patent publication, yielded several homologous proteins shown in Table 46C.

TABLE 46C
Geneseq Results for NOV46a

| | | | Identities/ | |
|---|---|---|---|---|
| | | | Similarities for | |
| Geneseq | Protein/Organism/Length | NOV46a Residues/ | the Matched | Expect |
| Identifier | [Patent #, Date] | Match Residues | Region | Value |
| AAU78297 | Human Retinoid X Receptor | 41 . . . 416 | 346/378 (91%) | 0.0 |
| | beta (RXRbeta) protein - | 156 . . . 533 | 352/378 (92%) | |
| | Homo sapiens, 533 aa. | | | |
| | [WO200218420-A2, | | | |
| | 07 MAR. 2002] | | | |
| AAR72483 | Human H-2RIIBP - Homo | 41 . . . 416 | 346/378 (91%) | 0.0 |
| | sapiens, 533 aa. | 156 . . . 533 | 352/378 (92%) | |
| | [US5403925-A, | | | |
| | 04 APR. 1995] | | | |
| AAR39468 | hRXR-beta1 - Homo sapiens, | 41 . . . 416 | 346/378 (91%) | 0.0 |
| | 533 aa. [WO9315216-A, | 156 . . . 533 | 352/378 (92%) | |
| | 05 AUG. 1993] | | | |
| AAR39469 | hRXR-beta2 - Homo sapiens, | 41 . . . 416 | 345/378 (91%) | 0.0 |
| | 510aa. [WO9315216-A, | 133 . . . 510 | 351/378 (92%) | |
| | 05 AUG. 1993] | | | |
| AAY21625 | Ligand binding domain of | 41 . . . 416 | 345/378 (91%) | 0.0 |
| | nuclear receptor hRXRbeta - | 148 . . . 525 | 351/378 (92%) | |
| | Homo sapiens, 525 aa. | | | |
| | [W09926966-A2, | | | |
| | 03 JUN. 1999] | | | |

In a BLAST search of public sequence datbases, the NOV46a protein was found to have homology to the proteins shown in the BLASTP data in Table 46D.

TABLE 46D
Public BLASTP Results for NOV46a

| | | | Identities/ | |
|---|---|---|---|---|
| Protein | | | Similarities for | |
| Accession | | NOV46a Residues/ | the Matched | Expect |
| Number | Protein/Organism/Length | Match Residues | Portion | Value |
| S37781 | retinoid X receptor beta - | 41 . . . 416 | 346/378 (91%) | 0.0 |
| | human, 533 aa. | 156 . . . 533 | 352/378 (92%) | |
| Q95L53 | Retinoid X receptor beta - | 41 . . . 416 | 346/378 (91%) | 0.0 |
| | Mustela vison (American | 148 . . . 525 | 352/378 (92%) | |
| | mink), 525 aa (fragment). | | | |
| P28702 | Retinoic acid receptor | 41 . . . 416 | 346/378 (91%) | 0.0 |
| | RXR-beta - Homo sapiens | 156 . . . 533 | 352/378 (92%) | |
| | (Human), 533 aa. | | | |
| A41651 | retinoic acid receptor | 41 . . . 416 | 341/378 (90%) | 0.0 |
| | coregulator - rat, 451 aa. | 74 . . . 451 | 349/378 (92%) | |
| D41727 | retinoid X receptor beta - | 41 . . . 416 | 341/378 (90%) | 0.0 |
| | mouse, 448 aa. | 71 . . . 448 | 349/378 (92%) | |

PFam analysis predicts that the NOV46a protein contains the domains shown in the Table 46E.

TABLE 46E
Domain Analysis of NOV46a

| | | Identities/ | |
|---|---|---|---|
| | | Similarities | |
| | | for the Matched | Expect |
| Pfam Domain | NOV46a Match Region | Region | Value |
| zf-C4 | 86 . . . 161 | 49/77 (64%) | 1.5e−54 |
| | | 73/77 (95%) | |
| hormone_rec | 227 . . . 409 | 74/207 (36%) | 3.3e−68 |
| | | 157/207 (76%) | |

### Example 47

The NOV47 clone was analyzed, and the nucleotide and encoded polypeptide sequences are shown in Table 47A.

TABLE 47A
NOV47 Sequence Analysis

| | |
|---|---|
| SEQ ID NO: 189 | 1229 bp |

| | |
|---|---|
| NOV47a, CCGAGACCATGGGGAACCTCGTGGCGCTGGTCCTGCTGGGGGTCGGCCTGTCCTTAGTCGGAGAT | |
| CG173347-01 | |
| DNA Sequence | GTTTCTGGCGTTTAGAGAAAGGGTGAATGCCTCTCGAGAAGTCGAGCCAGTAGAACCTGAAAACTGC |
| | CACCTTATTGAGCAACTTGAAAGTGGCTCTGAAGATATTGATATACTTCCTAGTGGGCTGGCTTTTA |
| | TCTCCAGTCTGCAGGTCTGTTGGAGTTTGCTGGAAGTCCACTCCAGACCCTGTTTGCCTCGGTATCA |
| | CCAGTGGAGGCTGCAGAACGGCAAATATTGCTGCCTGATTTTTCTTCTGGAAGCTTCATCCCAGAGG |
| | GGCATCCGCCTGTATGAGGGATTAAAATATCCACGCATGCCAAACTTTGCGCCAGATGAACCAGGAA |
| | AAATCTTCTTGATGGATCTGAATGAACAAAACCCAAGGGCACAAGCACTACAAATCAGTGGTGGATT |
| | TGACAAAGAATTATTTAATCCACATGGGATCAGTATTTTCATCGACAAAGACAATACTGTGTATCTT |
| | TATGTTGTGAATCATCCCCACATGAAGTCCACTGTGGAGATATTTAAATTTGAGGAACAACAACGTT |
| | CTCTGGTATACCTGAAAACTATAAAACATGAACTTCTCAAAAGTGTGAATGACATTGTGGTTCTTGG |
| | ACCAGAACAGTTCTATGCCACCAGAGACCACTATTTTACCAACTCCCTCCTGTCATTTTTTGAGATG |
| | ATCTTGGATCTTCGCTGGACTTATGTTCTTTTCTACAGCCCAAGGGAGGTTAAAGTGGTGGCCAAAG |
| | GATTTTGTAGTGCCAATGGGATCACAGTCTCAGCAGACCAGAAGTATGTCTATGTAGCTGATGTAGC |
| | AGCTAAGAACATTCACATAATGGAAAACATGATAACTGGGATTTAACTCAACTGAAAGGTGATACAG |
| | TTGGGCACCTTAGTGGATAACCTGACTGTCGATCCTGCCACACGAGACATTTTGGCAGGATGCCATC |
| | CTAATCCTATGAAGCTACTGAACTATAACCCTGAGGACCCTCCAGGATCAGAAGTACTTCGCATCCA |
| | GAATGTTTTGTCTGAGAAGCCCAGGGTGAGCACCGTGTATGCCAACAATGGCTCTGTGCTTCAAGGC |
| | ACCTCTGTGGCTTCTGTGTACCATGGGAAAATTCTCATACGCACCGTATTTCNCAAAACTCTGTACT |
| | GTGAGCTCTAGACTCTAGATAGT |

| | | |
|---|---|---|
| ORF Start: ATG at 9 | | ORF Stop: TAG at 1215 |
| SEQ ID NO: 190 | 402 aa | MW at 45160.5 kD |

| | |
|---|---|
| NOV47a, MGKLVALVLLGVGLSLVGEMFLAFRERVNASREVEPVEPENCHLIEELESGSEDIDILPSGLAFISS | |
| CG173347-01 | |
| Protein Sequence | LQVCWSLLEVHSRPCLPGYHQWRLQNGKYCCLIFLLEASSQRGIRLYEGLKYPGMPNFAPDEPGKIF |
| LMDLNEQNPRAQALEISGGFDKELFNPHGISIFIDKDNTVYLYVVNHPHMKSTVEIFKFEEQQRSLV | |
| YLKTIKHELLKSVNDTVVLGPEQFYATRDHYFTNSLLSFFEMILDLRWTYVLFYSPREVKVVAKGFC | |
| SANGITVSADQKYVYVADVAAKNIHIMEKHDNWDLTQLKVIQLGTLVDNLTVDPATGDILAGCHPNP | |
| | MXLLNYNPEDPPGSEVLRIQNVLSEKPRVSTVYANNGSVLQGTSVASVYHGKTLIGTVFXKTLYCEL |

Further analysis of the NOV47a protein yielded the following properties shown in Table 47B.

TABLE 47B
Protein Sequence Properties NOV47a

| | |
|---|---|
| PSort analysis: | 0.8200 probability located in outside; |
| | 0.1900 probability located in lysosome (lumen); |
| | 0.1000 probability located in endoplasmic |
| | reticulum (membrane); |
| | 0.1000 probability located in endoplasmic |
| | reticulum (lumen) |
| SignalP analysis: | Cleavage site between residues 31 and 32 |

A search of the NOV47a protein against the Geneseq database, a proprietary database that contains sequences published in patents and patent publication, yielded several homologous proteins shown in Table 47C.

TABLE 47C
Geneseq Results for NOV47a

| | | NOV47a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB97287 | Novel human protein SEQ ID | 1 . . . 402 | 352/402 (87%) | 0.0 |
| | NO: 555 - Homo sapiens, 354 | 1 . . . 354 | 352/402 (87%) | |
| | aa. [WO200222660-A2, | | | |
| | 21-MAR-2002] | | | |
| AAG75494 | Human colon cancer antigen | 2 . . . 402 | 352/401 (87%) | 0.0 |
| | protein SEQ ID NO: 6258 - | 18 . . . 370 | 352/401 (87%) | |
| | Homo sapiens, 370 aa. | | | |
| | [WO200122920-A2, | | | |
| | 05-APR-2001] | | | |
| ABG08350 | Novel human diagnostic | 1 . . . 402 | 330/407 (81%) | e−178 |
| | protein #8341 - Homo | 24 . . . 382 | 333/407 (81%) | |
| | sapiens, 382 aa. | | | |
| | [WO200175067-A2, | | | |
| | 11-OCT-2001] | | | |
| AAU11925 | Protein sequence of rabbit | 1 . . . 402 | 294/403 (72%) | e−164 |
| | paraoxonase-3 (PON3) | 1 . . . 355 | 318/403 (77%) | |
| | mutant D324N - Oryctolagus | | | |
| | cuniculus, 355 aa. | | | |
| | [WO200190336-A2, | | | |
| | 29-NOV-2001] | | | |
| AAU11922 | Protein sequence of rabbit | 1 . . . 402 | 294/403 (72%) | e−164 |
| | paraoxonase-3 (PON3) | 1 . . . 355 | 318/403 (77%) | |
| | mutant N169D - Oryctolagus | | | |
| | cuniculus, 355 aa. | | | |
| | [WO200190336-A2, | | | |
| | 29-NOV-2001] | | | |

In a BLAST search of public sequence datbases, the NOV47a protein was found to have homology to the proteins shown in the BLASTP data in Table 47D.

TABLE 47D
Public BLASTP Results for NOV47a

| | | NOV47a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q15166 | Serum | 1 . . . 402 | 354/402 (88%) | 0.0 |
| | paraoxonase/arylesterase 3 | 1 . . . 354 | 354/402 (88%) | |
| | (EC 3.1.1.2) (EC 3.1.8.1) | | | |
| | (PON 3) (Serum | | | |
| | aryldiakylphosphatase 3) | | | |
| | (A-esterase 3) (Aromatic | | | |
| | esterase 3) - Homo sapiens | | | |
| | (Human), 354 aa. | | | |
| Q9BZH9 | Paraoxanase-3 - Homo | 1 . . . 402 | 351/402 (87%) | 0.0 |
| | sapiens (Human), 354 aa | 1 . . . 354 | 351/402 (87%) | |
| | (fragment). | | | |
| Q9BGN0 | Paraoxonase 3 - Oryctolagus | 1 . . . 402 | 293/402 (72%) | e−164 |
| | cuniculus (Rabbit), 354 aa. | 1 . . . 354 | 318/402 (78%) | |
| Q62087 | Serum | 1 . . . 402 | 283/402 (70%) | e−158 |
| | paraoxonase/arylesterase 3 | 1 . . . 354 | 314/402 (77%) | |
| | (EC 3.1.1.2) (EC 3.1.8.1) | | | |
| | (PON 3) (Serum | | | |
| | aryldiakylphosphatase 3) | | | |
| | (A-esterase 3) (Aromatic | | | |
| | esterase 3) - Mus musculus | | | |
| | (Mouse), 354 aa. | | | |
| Q90952 | Serum | 1 . . . 402 | 230/402 (57%) | e−131 |
| | paraoxonase/arylesterase 2 | 1 . . . 354 | 287/402 (71%) | |
| | (EC 3.1.1.2) (BC 3.1.8.1) | | | |
| | (PON 2) (Serum | | | |
| | aryldiakylphosphatase 2) | | | |
| | (A-esterase 2) (Aromatic | | | |
| | esterase 2) - Gallus gallus | | | |
| | (Chicken), 354 aa. | | | |

PFam analysis predicts that the NOV47a protein contains the domains shown in the Table 47E.

TABLE 47E
Domain Analysis of NOV47a

| | NOV47a | Identities/ | |
|---|---|---|---|
| | Match | Similarities | |
| Pfam Domain | Region | for the Matched Region | Expect Value |
| Arylesterase | 2 . . . 402 | 230/422 (55%) | 1.2e−190 |
| | | 348/422 (82%) | |

### Example 48

The NOV48 clone was analyzed, and the nucleotide and encoded polypeptide sequences are shown in Table 48A.

TABLE 48A
NOV48 Sequence Analysis

| | SEQ ID NO: 191 | 2109 bp | |
|---|---|---|---|
| NOV48a, | CTTCCATACCTCCCCGGCTCCGCTCGGTTCCTGGCCACCCCGCAAGCCCCTGCCCAGGTGCCATGGC | | |
| CG56234-01 | | | |
| DNA Sequence | CGCATTGTACCGCCCTGGCCTGCGGCTTAACTGGCATGGGCTGAGCCCCTTGGGCTGGCCATCATGC | | |
| | CGTAGCATCCAGACCCTGCGAGTGCTTAGTGCAGATCTGGGCCAGCTTCCCACTGGCATTCGAGATT | | |
| | TTGTAGAGCACAGTGCCCGCCTGTGCCAACCAGAGGGCATCCACATCTGTGATGGAACTGAGCCTGA | | |
| | GAATACTGCCACACTGACCCTGCTGGAGCAGCAGGCCCTCATCCGAAAGCTCCCCAAGTACAATAAC | | |
| | TGCTGGCTGGCCCGCACAGACCCCAAGGATGTGGCACGAGTAGAGAGCAAGACGGTGATTGTAACTC | | |
| | CTTCTCAGCGGGACACGGTACCACTCCCGCCTGGTGGCGCCCGTGGGCAGCTGGGCAACTGGATGTC | | |
| | CCCAGCTGATTTCCAGCGAGCTGTGGATGAGAGGTTTCCAGGCTGCATGCAGGGCCGCACCATGTAT | | |
| | GTGCTTCCATTCAGCATGGGTCCTGTGGGCTCCCCGCTGTCCCGCATCGGGGTGCAGCTCACTGACT | | |
| | CAGCCTATGTGGTGGCAAGCATGCGTATTATGACCCGACTGGGGACACCTGTGCTTCAGGCCCTGGC | | |
| | AGATGGTGACTTTGTCAAGTGTCTGCACTCCGTGGGCCAGCCCCTGACAGGACAAGGGGAGCCAGTG | | |
| | AGCCAGTGGCCGTGCAACCCAGAGAAAACCCTGATTGGCCACGTGCCCGACCAGCGGGAGATCATCT | | |
| | CCTTCGGCAGCGGCTATGGTGGCAACTCCCTCCTGGGCAAGAAGTGCTTTGCCCTACGCATCGCCTC | | |
| | TCGGCTGGCCCGGGATGAGGGCTGGCTGGCAGAGCACATGCTGATCCTGGGCATCACCAGCCCTGCA | | |
| | GGGAAGAAGCGCTATGTGGCAGCCGCCTTCCCTAGTGCCTGTGGCAAGACCAACCTGGCTATGATGC | | |
| | GGCCTGCACTGCCAGGCTGGAAAGTGGAGTGTGTGGGGGATCATATTGCTTGCATGAGGTTTGACAG | | |
| | TGAAGGTCGACTCCCGGCCATCAACCCTGAGAACGGCTTCTTTGGGGTTGCCCCTGGTACCTCTGCC | | |
| | ACCACCAATCCCAACGCCATGGCTACAATCCAGAGTAACACTATTTTTACCAATGTGGCTGAGACCA | | |
| | GTGATCGTGGCCTGTACTGGGAGGGCATTGACCAGCCTCTTCCACCTGGTGTTACTGTGACCTCCTG | | |
| | GCTCGGCAAACCCTGGAAATCTGGTGACAAGGAGCCCTGTGCACATCCCAACTCTCGATTTTGTGCC | | |
| | CCGGCTCGCCAGTGCCCCATCATGGACCCAGCCTGGGAGGCCCCAGAGGGTGTCCCCATTGACGCCA | | |
| | TCATCTTTGGTGGCCGCAGACCCAAAGGGGTACCCCTGGTATACGACGCCTTCAACTCGCGTCATGG | | |
| | GGTGTTTGTGGGCAGCGCCATGCGCTCTGAGTCCACTGCTGCAGCAGAACACAAAGGGAAGATCATC | | |
| | ATGCACGACCCATTTGCCATGCGGCCCTTTTTTGGCTACAACTTCGGGCACTACCTGAAACACTGGC | | |
| | TGAGCATGGAAGGGCGCAACGGGGCCCAGCTGCCCCGTATCTTCCATGTCAACTGGTTCCGGCGTGA | | |
| | CGAGGCAGGGCACTTCCTGTGGCCAGGCTTTGGGGAGAATGCTCGGGTGCTAGACTGGATCTGCCGG | | |
| | CGGTTAGAGGGGGAGGACAGTGCCCGAGAGACACCCATTGGGCTGGTACCAAAGGAAGGAGCCTTGG | | |
| | ATCTCAGCGGCCTCAGAGCTATAGACACCACTCAGCTGTTCTCCCTCCCCAAGGACTTCTGGGAACA | | |
| | GGAGGTTCGTGACATTCGGAGCTACCTGACAGAGCAGGTCAACCAGGATCTGCCCAAAGAGGTGTTC | | |
| | GCTGAGCTTGAGGCCCTGGAGAGACGTCTGCACAAAATGTGACCTCAGGCCCTAGTCTAGCAAGAGG | | |
| | ACATAGCACCCTCATCTGGGAATAGGGAAGGCACCTTGCAGAAAATATGAGCAATTTGATATTAACT | | |
| | AACATCTTCAATGTGCCATAGACCTTCCCACA | | |
| | ORF Start: ATG at 63 | | ORF Stop: TGA at 1983 |
| | SEQ ID NO: 192 | 640 aa | MW at 70688.2 kD |
| NOV48a, | MAALYRPGLRLNWHGLSPLGWFSCRSIQTLRVLSGDLGQLPTGIRDFVEHSARLCQPEGIHICDGTE | | |
| CG56234-01 | | | |
| Protein Sequence | AENTATLTLLEQQGLIRKLPKYNNCWLARTDFKDVARVESKTVIVTPSQRDTVPLPPGGARGQLGNW | | |
| | MSPADFQRAVDERFPGCMQGRTMYVLPFSMGPVGSPLSRIGVQLTDSAYVVASMRIMTRLGTPVLQA | | |
| | LGDGDFVKCLHSVGQPLTGQGEPVSQWPCNPEKTLIGHVPDQREIISFGSGYGGNSLLGKKCFALRI | | |
| | ASRLARDEGWLAEHMLILGITSPAGKKRYVAAAFPSACGKTNLAMMRPALPGWKVECVGDDIAWMRF | | |
| | DSECRLRAINPENGFFGVAPGTSATTNPNAMATIQSNTTFTNVAETSDGGVYWEGIDQPLPPGVTVT | | |
| | SWLGKPWKSGDKEPCAHPNSRFCAPARQCPIMDPAWEAPEGVPIDAIIFGGRRPKGVPLVYEAFNWR | | |
| | HGVFVGSAMRSESTAAAEHKGKIIMHDPFAMRPFFGYNFGHYLEHWLSMEGRKGAQLPRIFHVNWFR | | |
| | RDEAGHFLWPGFGENARVLDWICRRLEGEDSARETPIGLVPKEGALDLSGLRAIDTTQLFSLPKDFW | | |
| | EQEVRDIRSYLTEQVNQDLPKEVLAELEALERRVHKM | | |
| | SEQ ID NO: 193 | 2069 bp | |
| NOV48b, | CCCGCCTTCCATACCTCCCCGGCTCCGCTCCGTTCCTGGCCACCCCGCAGCCCCTGCCCAGGTCCCA | | |
| CG56234-02 | | | |
| DNA Sequence | TGGCCGCATTGTACCGCCCTGGCCTGCGGCTTAACTGGCATGGGCTGAGCCCCTTGGGCTGGCCATC | | |
| | ATGCCGTAGCATCCAGACCCTGCGAGTGCTTAGTGGAGATCTGGGCCAGCTTCCCACTGGCATTCGA | | |
| | GATTTTGTAGACCACAGTGCCCGCCTGTGCCAACCAGAGAACATCCACATCTGTGATGGAACTGAGG | | |
| | CTGAGAATACTGCCACACTGACCCTGCTCGAGCAGCAGGGCCTCATCCGAAAGCTCCCCAAGTACAA | | |
| | TAACTGCTGGCTGGCCCGCACAGACCCCAAGGATGTCGCACGAGTAGAGAGCAAGACGGTGATTGTA | | |
| | ACTCCTTCTCAGCGGGACACGGTACCACTCCCGCCTGGTGGGGCCTGTGGGCAGCTGGGCAACTGGA | | |
| | TGTCCCCAGCTGATTTCCAGCGAGCTGTGGATCAGAGGTTTCCAGGCTGCATGCAGGGCCGCACCAT | | |
| | GTATGTGCTTCCATTCAGCATGGGTCCTGTCGGCTCCCCGCTGTCCCGCATCGGGGTGCAGCTCACT | | |
| | GACTCAGCCTATGTGGTGCCAACCATGCGTATTATAACCCGACTGGCCACACCTGTGCTTCAGGCCC | | |
| | TGGGAGATCGTGACTTTGTCAAGTGTCTGCACTCCGTGGGCCAGCCCCTGACAGGACAAGGGGAGCC | | |
| | AGTGAGCCAGTGGCCGTGCAACCCAGAGAAAACCCTGATTGGCCACGTGCCCGACCAGCGGGAGATC | | |
| | ATCTCCTTCGGCAGCGGCTATGGTGGCAACTCCCTGCTGGGCAAGAAGTGCTTTGCCCTACGCATCG | | |
| | CCTCTCGGCTGGCCCGGGATGAGGGCTCGCTGGCAGAGCACATGCTGATCCTGGGCATCACCAGCCC | | |
| | TGCAGGGAAGAAGGCGCTATGTGCAGCCGCCTTCCCTAGTGCCTGTGGCAAGACCAACCTGGCTATG | | |
| | ATGCGGCCTGCACTGCCAGGCTGGAAAGTGGAGTGTGTCGGGGATGATATTGCTTGGATGAGGTTTG | | |
| | ACAGTGAAGGTCGACTCCGGGCCATCAACCCTGAGAACGGCTTCTTTGGGGTTGCCCCTGGTACCTC | | |
| | TGCCACCACCAATCCCAACGCCATGGCTACAATCCAGAGTAACACTATTTTTACCAATGTGGCTGAG | | |
| | ACCAGTCATGGTGGCGTGTACTGGGAGGGCATTGACCAGCCTCTTCCACCTGGTGTTACTGTGACCT | | |
| | CCTGGCTGGGCAAACCCTGGAAACCTGGTGACAAGGAGCCCTGTGCACATCCCAACTCTCGATTTTG | | |
| | TGCCCCGGCTCCCCAGTGCCCCATCATGGACCCAGCCTGGGAGGCCCCAGAGGGTGTCCCCATTGAC | | |
| | GCCATCATCTTTGGTGGCCGCAGACCCAAAGGGAAGATCATCATGCACGACCCATTTGCCATGCGGC | | |
| | CCTTTTTTGGCTACAACTTCGGGCACTACCTGGAACACTGGCTGAGCATGGAAGGGCGCAAGGGGGC | | |
| | CCAGCTGCCCCGTATCTTCCATGTCAACTGGTTCCGGCGTGACGAGGCAGGGCACTTCCTGTGGCCA | | |
| | GGCTTTGGGGAGAATGCTCGCGTGCTAGACTGGATCTGCCGGCGGTTAGAGAAGGAGGACAGTGCCC | | |
| | GAGAGACACCCATTGGGCTGGTGCCAAAGGAAGGAGCCTTGGATCTCAGCGGCCTCAGAGCTATAGA | | |
| | CACCACTCAGCTGTTCTCCCTCCCCAAGGACTTCTGGGAACAGGAGGTTCGTGACATTCGGAGCTAC | | |
| | CTGACAGACCAGGTCAACCAGGATCTGCCCAAAGAGGTGTTGGCTGAGCTTGAGGCCCTGGAGAGAC | | |
| | GTGTGCACAAAATGTGACCTGAGGCCTAGTCTAGCAAGAGGACATAGCACCCTCATCTGGGAATAGG | | |
| | GAAGGCACCTTGCAGAAAATATGAGCAATTGATATTAACTAACATCTTCAATGTGCCATAGACCTTC | | |
| | CCACAAAGACTGTCCAATAATAAGAGATGCTTATCTATTTTAAAAAAAAAAAAAAAAAA | | |
| | ORF Start: ATG at 67 | | ORF Stop: TGA at 1891 |
| | SEQ ID NO: 194 | 608 aa | MW at 67027.1 kD |

| | |
|---|---|
| NOV48b, | MAALYRPGLRLNWHGLSPLGWPSCRSIQTLRVLSGDLGQLPTGIRDFVEHSARLCQPEGIHICDGTE |
| CG56234-02 | |
| Protein Sequence | AENTATLTLLEQQGLIRKLPKYNNCWLARTDPKDVARVESKTVIVTPSQRDTVPLPPGGACGQLGNW |
| | MSPADFQRAVDERFPGCMQGRTMYVLPFSMGPVGSPLSRIGVQLTDSAYVVASMRIMTRLGTPVLQA |
| | LGDGDFVKCLHSVGQPLTGQGEPVSQWPCNPEKTLTGHVPDQREIISFGSGYGGNSLLGKKCFALRI |
| | ASRLARDEGWLAEHMLILGITSPAGKKALCAAAFPSACGKTNLAMMRPALPGWKVECVGDDIAWMRE |
| | DSEGRLRAINPENGFFGVAPGTSATTNPNAMATIQSNTIFTNVAETSDGGVYWEGIDQPLPPGVTVT |
| | SWLGKPWKPGDKEPCAHPNSRFCAPARQCPIMDPAWEAPEGVPIDAIIFGGRRPKGKIIMHDPEAMR |
| | PFFGYNFGHYLEHWLSMEGRKGAQLPRIFHVNWFRRDEAGHFLWPGFGENARVLDWICRRLEGEDSA |
| | RETPIGLVPKECALDLSGLRAIDTTQLFSLPKDFWEQEVRDIRSYLTEQVNQDLPKEVLAELEALER |
| | RVHKM |

Sequence comparison of the above protein sequences yields the following sequence relationships shown in Table 48B.

TABLE 48B
Comparison of NOV48a against NOV48b.

| Protein | NOV48a Residues/ | Identities/ |
|---|---|---|
| Sequence | Match Residues | Similarities for the Matched Region |
| NOV48b | 1 . . . 640 | 577/640 (90%) |
| | 1 . . . 608 | 577/640 (90%) |

Further analysis of the NOV48a protein yielded the following properties shown in Table 48C.

TABLE 48C
Protein Sequence Properties NOV48a

| | |
|---|---|
| PSort analysis: | 0.6402 probability located in microbody (peroxisome); |
| | 0.3000 probability located in nucleus; 0.2412 |
| | probability located in lysosome (lumen); 0.1000 |
| | probability located in mitochondrial matrix space |
| SignalP analysis: | No Known Signal Sequence Predicted |

A search of the NOV48a protein against the Geneseq database, a proprietary database that contains sequences published in patents and patent publication, yielded several homologous proteins shown in Table 48D.

TABLE 48D
Geneseq Results for NOV48a

| | | NOV48a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| AAY80296 | Human mitochondrial | 1 . . . 640 | 634/640 (99%) | 0.0 |
| | phosphoenolpyruvate | 1 . . . 640 | 634/640 (99%) | |
| | carboxykinase SEQ ID NO: 1 - | | | |
| | Homo sapiens, 640 aa. | | | |
| | [US6030837-A, | | | |
| | 29-FEB-2000] | | | |
| AAB71890 | Mouse PEPCK-cytosolic | 31 . . . 640 | 440/610 (72%) | 0.0 |
| | protein - Mus musculus, 622 | 14 . . . 622 | 519/610 (84%) | |
| | aa. [US6187545-B1, | | | |
| | 13-FEB-2001] | | | |
| AAB71880 | Human PEPCK-cytosolic | 31 . . . 640 | 438/610 (71%) | 0.0 |
| | protein - Homo sapiens, 622 | 14 . . . 622 | 517/610 (83%) | |
| | aa. [US6187545-B1, | | | |
| | 13-FEB-2001] | | | |
| ABB65318 | Drosophila melanogaster | 27 . . . 640 | 394/616 (63%) | 0.0 |
| | polypeptide SEQ ID NO: | 35 . . . 647 | 480/616 (76%) | |
| | 22746 - Drosophila | | | |
| | melanogaster, 647 aa. | | | |
| | [WO200171042-A2, | | | |
| | 27-SEP-2001] | | | |
| ABB65322 | Drosophila melanogaster | 30 . . . 640 | 402/613 (65%) | 0.0 |
| | polypeptide SEQ ID NO: | 29 . . . 638 | 469/613 (75%) | |
| | 22758 - Drosophila | | | |
| | melanogaster, 638 aa. | | | |
| | [WO200171042-A2, | | | |
| | 27-SEP-2001] | | | |

In a BLAST search of public sequence datbases, the NOV48a protein was found to have homology to the proteins shown in the BLASTP data in Table 48E.

TABLE 48E
Public BLASTP Results for NOV48a

| | | NOV48a | Identities/ | |
|---|---|---|---|---|
| Protein | | Residues/ | Similarities for | |
| Accession | | Match | the Matched | Expect |
| Number | Protein/Organism/Length | Residues | Portion | Value |
| Q16822 | Phosphoenolpyruvate | 1 . . . 640 | 635/640 (99%) | 0.0 |
| | carboxykinase, mitochondrial | 1 . . . 640 | 635/640 (99%) | |
| | precursor [GTP] (EC 4.1.1.32) | | | |
| | (Phosphoenolpyruvate | | | |
| | carboxylase) (PEPCK-M) - | | | |
| | Homo sapiens (Human), 640 aa. | | | |
| S69546 | phosphoenolpyruvate | 1 . . . 640 | 634/640 (99%) | 0.0 |
| | carboxykinase (GTP) (EC | 1 . . . 640 | 634/640 (99%) | |
| | 4.1.1.32) precursor, | | | |
| | mitochondrial - human, 640 | | | |
| | aa. | | | |
| Q91Z10 | Similar to | 1 . . . 640 | 590/640 (92%) | 0.0 |
| | phosphoenolpyruvate | 1 . . . 640 | 609/640 (94%) | |
| | carboxykinase 2 | | | |
| | (mitochondrial) - Mus | | | |
| | musculus (Mouse), 640 aa. | | | |
| Q8R3X7 | Similar to RIKEN cDNA | 106 . . . 640 | 504/535 (94%) | 0.0 |
| | 9130022B02 gene - Mus | 1 . . . 535 | 518/535 (96%) | |
| | musculus (Mouse), 535 aa (fragment). | | | |
| P07379 | Phosphoenolpyruvate | 31 . . . 640 | 441/610 (72%) | 0.0 |
| | carboxykinase, cytosolic | 14 . . . 622 | 520/610 (84%) | |
| | [GTP] (EC 4.1.1.32) | | | |
| | (Phosphoenolpyruvate | | | |
| | carboxylase) (PEPCK-C) - | | | |
| | Rattus norvegicus (Rat), 622 | | | |
| | aa. | | | |

PFam analysis predicts that the NOV48a protein contains the domains shown in the Table 48F.

TABLE 48F
Domain Analysis of NOV48a

| | | Identities/ | |
|---|---|---|---|
| Pfam | | Similarities | Expect |
| Domain | NOV48a Match Region | for the Matched Region | Value |
| PEPCK | 46 . . . 640 | 445/608 (73%) | 0 |
| | | 591/608 (97%) | |

### Example 49

The NOV49 clone was analyzed, and the nucleotide and encoded polypeptide sequences are shown in Table 49A.

TABLE 49A
NOV49 Sequence Analysis

| | SEQ ID NO: 195 | 1202 bp | |
|---|---|---|---|
| NOV49a, | TGTAAGCGATCTGGTTCCCACCTCAGCCTCCCGAGTAGTGTCTTCAGGCCTATGGAGAGCAGCTTGC | | |
| CG56836-01 | | | |
| DNA Sequence | GTCGGCTGGGCCTGCAGTACCTGGTTTGCATAGATGATTCGCAGCTGGATCTAGGATCCGGCTTCCA | | |
| | ACATGTGGCAGCTCTCGGCCTCCCTCTGCTGCCTGCTGGTGTTGGCCAATGCCCGGAGCAGGCCCT | | |
| | TTTCCATCCCCTGTCGGATGAGCTGGTCAACTATGTCAACAAACGGAATACCACGTGGCAGGCCGGG | | |
| | CACAACTTCTACAACGTGGACATGAGCTACTTGAAGAGGCTATGTGGTACCTTCCTCGGTGGGCCCA | | |
| | AGCCACCCCAGAGAGTTATGTTTACCGAGGACCTGAAGCTGCCTGCAAGCTTCGATCCACGGGAACA | | |
| | ATGGCCACAGTGTCCCACCATCAAAGAGATCAGAGACCAGGGCTCCTCTGGCTCCTGCTGGGCCTTC | | |
| | GGGGCTGTGGAAGCCATCTCTGACCGGATCTGCATCCACACCAATGCGCACGTCAGCGTGGAGGTGT | | |
| | CGGCGGAGGACCPGCTCACATGCTGTGGCAGCATGTGTGGGGACGGCTGTAATGGTCGCTATCCTGC | | |
| | TGAAGCTTGCAACTTCTGGACAAGAAAAGGCCTCGTTTCTGGTGGCCTCTATGAATCCCATGTAGGG | | |
| | TGCAGACCGTACTCCATCCCTCCCTGTGAGCACCACGTCAACGGCTCCCGGCCCCCATGCACGGGGG | | |
| | AGGGAGATACCCCCAAGTGTAGCAAGATCTGTGAGCCTGGCTACAGCCCGACCTACAAACAGGACAA | | |
| | GCACTACGGATACAATTCCTACAGCGTCTCCAATAGCGAGAAGGACATCATGGCCGAGATCTACAAA | | |
| | AACGGCCCCGTGGAGGGAGCTTTCTCTGTGTATTCGGACTTCCTGCTCTACAAGTCAGGAGTGTACC | | |
| | AACACGTCACCGGAGAGATGATGGGTGGCCATGCCATCCGCATCCTGGGCTGGGGAGTGGAGAATGG | | |
| | CACACCCTACTGGCTGGTTGCCAACTCCTCGAACACTGACTGGGGTGACAATGGCTTCTTTAAAATA | | |
| | CTCAGAGGACAGGATCACTGTGGAATCGAATCAGAAGTGGTGGCTGGAATTCCACGCACCGATCAGT | | |
| | ACTGGGAAAAGATCTAATCTGCCGTGGGCCTGTCGTGCCAGTCCTGGGGGCGAGATCGCGGTA | | |
| | ORF Start: ATG at 137 | | ORF Stop: TAA at 1154 |
| | SEQ ID NO: 196 | 339 aa | MW at 37821.3 kD |
| NOV49a, | MWQLWASLCCLLVLANARSRPSFHPLSDELVNYVNKRNTTWQAGHNFYNVDMSYLKRLCGTFLGGPK | | |
| CG56836-01 | | | |
| Protein Sequenc PPQRVMFTEDLKLPASFDAREQWPQCPTIKEIRDQGSCGSCWAFGAVEAISDRICIHTNAHVSVEVS | | | |
| | AEDLLTCCGSMCGDGCNGGYPAEAWNFWTRKGLVSGCLYESHVGCRPYSIPPCEHHVNGSRPPCTGE | | |
| | CDTPKCSKICEPGYSPTYKQDKHYGYNSYSVSNSEKDIMAEIYKNGPVEGAFSVYSDFLLYKSGVYQ | | |
| | HVTGEMMGGHAIRILGWGVENGTPYWLVANSWNTDWGDNGFFKILRGQDUCGIESEVVAGIPRTDQY | | |
| | WEKI | | |
| | SEQ ID NO: 197 | 723 bp | |
| NOV49b, | ACATGGTGGATCTAGGATCCGGCTTCCAACATGTGGCAGCTCTGGGCCTCCCTCTGCTGCCTGCTGG | | |
| CG56836-02 | | | |
| DNA Sequence TGTTGGCCAATGCCCGGAGCAGGCCCTCTTTCCATCCCCTGTCGGATGAGCTGGTCAACTATGTCAA | | | |
| | CAAACGGAATACCACGTGGCAGGCCGGGCACAACTTCTACAACGTGGACATGAGCTACTTGAAGAGG | | |
| | CTATGTGGTACCTTCCTGGGTGGGCCCAAGCCACCCCAGAGAGTTATGTTTACCGAGGACCTGAAGC | | |
| | TGCCTGCAAGCTTCGATGCACGGGAACAATGGCCACAGTGTCCCACCATCAAAGAGATCAGAGACCA | | |
| | GGGCTCCTGTGGCTCCTGCTGGGCCTTCGCGGCTGTGGAAGCCATCTCTGACCGGATCTCCATCCAC | | |
| | ACCAATGCGCACGTCAGCGTGGAGGTGTCGGCGGAGGACCTGCTCACCTGCCTGCTCTACAAGTCAG | | |
| | GAGTGTACCAACACGTCACCGGAGAGATGATGGGTGGCCATGCCATCCGCATCCTGGGCTGGGGAGT | | |
| | GGAGAATGGCACACCCTACTGGCTGGTTGCCAACTCCTGGAACACTGACTGGGGTGACAATGGCTTC | | |
| | TTTAAAATACTCAGAGGACAGGATCACTGTGGAATCGAATCACAAGTGGTGGCTGGAATTCCACGCA | | |
| | CCGATCAGTACTGGGAAAAGATCTAATCTGCCGTGGGCCTGTCGTGCCAAACC | | |
| | ORF Start: ATG at 31 | | ORF Stop: TAA at 694 |
| | SEQ ID NO: 198 | 221 aa | MW at 24974.2 kD |
| NOV49b, | MWQLWASLCCLLVLANARSRPSFHPLSDELVNYVNKRNTTWQAGUNFYNVDMSYLKRLCGTFLGGPK | | |
| CG56836-02 | | | |
| Protein Sequence | PPQRVMFTEDLKLPASFDAREQWPQCPTIKEIRDQGSCGSCWAFGAVEAISDRICIHTNAHVSVEVS | | |
| | AEDLLTCLLYKSGVYQHVTGEMMGGHAIRILGWGVENGTPYWLVANSWNTDWGDNGFFKILRCQDHC | | |
| | GIESEVVAGIPRTDQYWEKI | | |
| | SEQ ID NO: 199 | 1028 bp | |
| NOV49c, | TGTAAGCGATCTGGTTCCCACCTCAGCCTCCCGAGTAGTCTCTTCAGGCCTATGGAGAGCAGCTTGC | | |
| CG56836-03 | | | |
| DNA Sequence | GTGGGCTGGGCCTGCAGTACCTGGTTTGCATAGATGATTGGCAGGTGGATCTACGATCCGGCTTCCA | | |
| | ACATGTGGCAGCTCTGCGCCTCCCTCTGCTGCCTGCTCGTGTTGGCCAATGCCCGGAGCAGGCCCTC | | |
| | TTTCCATCCCCTGTCGGATGAGCTGGTCAACTATGTCAACAAACGGAATACCACGTGGCAGGCCGGG | | |
| | CACAACTTCTACAACGTGCACATGAGCTACTTGAAGAGGCTATGTGGTACCTTCCTGGGTAAGCCCA | | |
| | AGCCACCCCAGAGAGTTATGTTTACCGAGCACCTGAAGCTGCCTGCAAGCTTCGATGCACGGGAACA | | |
| | ATGGCCACAGTGTCCCACCATCAAAGAGATCAGAGACCAGGGCTCCTGTGGCTCCTGCTGGGCCTTC | | |
| | GGGGCTGTGGAAGCCATCTCTGACCGGATCTGCATCCACACCAATGCGCACGTCAGCGTGGACGTGT | | |
| | CGGCGGAGGACCTCCTCACATGCTGTGGCAGCATGTGTGGCGACGGCTGTAATGGTGGCTATCCTGC | | |
| | TGAAGCTTGGAACTTCTGGACAAGAAAAGGCCTGGTTTCTGGTGCCCTCTATGAATCCAATAGCGAG | | |
| | AAGGACATCATGGCCGAGATCTACAAAAACGGCCCCGTGGAGGGAGCTTTCTCTGTGTATTCGGACT | | |
| | TCCTGCTCTACAAGTCAGGAGTGTACCAACACGTCACCGGAGAGATGATGGGTGGCCATGCCATCCG | | |
| | CATCCTGGGCTGGGGAGTGGAGAATGGCACACCCTACTGGCTGGTTGCCAACTCCTGGAACACTGAC | | |
| | TGGGGTGACAATGGCTTCTTTAAAATACTCAGAGGACAGGATCACTGTGGAATCGAATCAGAAGTGG | | |
| | TGGCTGGAATTCCACGCACCGATCAGTACTGGGAAAAGATCTAATCTGCCGTGGGCCTGTCGTGCCA | | |
| | GTCCTGGGGGCGAGATCGGGGTA | | |
| | ORF Start: ATG at 137 | | ORF Stop: TAA at 980 |
| | SEQ ID NO: 200 | 281 aa | MW at 31423.2 kD |
| NOV49c, | MWQLWASLCCLLVLANARSRPSFHPLSDELVNYVNKRNTTWQAGHNFYNVDMSYLKRLCGTFLGGPK | | |
| CG56836-03 | | | |
| Protein Sequence | PPQRVMFTEDLKLPASFDAREQWPQCPTIKEIRDQGSCGSCWAFCAVEAISDRICIHTNAHVSVEVS | | |
| | AEDLLTCCGSMCGDGCNGGYPAEAWNFWTRKGLVSGGLYESNSEKDIMAEIYKNGPVEGAFSVYSDF | | |
| | LLYZSGVYQHVTGEMMGGNAIRILGWGVENGTPYWLVANSWNTDWGDNGFFKILRGQDHCGIESEVV | | |
| | AGIPRTDQYWEKI | | |
| | SEQ ID NO: 201 | 1028 bp | |
| NOV49d, | TGTAAGCGATCTGGTTCCCACCTCAGCCTCCCGAGTAGTGTCTTCAGGCCTATGGAGAGCAGCTTGC | | |
| CG56836-04 | | | |
| DNA Sequence | GTCGGCTGCGCCTGCAGTACCTGGTTTGCATAGATGATTGGCAGGTGGATCTACGATCCGGCTTCCA | | |
| | ACATGTGGCAGCTCTGGGCCTCCCTCTGCTGCCTGCTGGTGTTGGCCAATGCCCGGAGCAGGCCCTC | | |
| | TTTCCATCCCCTGTCGGATGAGCTGGTCAACTATGTCAACAGACGGAATACCACGTGGCAGGCCGGG | | |
| | CACAACTTCTACAACGTGGACATGAGCTACTTGAAGAGGCTATGTCGTACCTTCCTGGGTGGGCCCA | | |
| | AGCCACCCCAGAGAGTTATGTTTACCGAGGACCTGAAGCTGCCTGCAAGCTTCGATGCACGGGAACA | | |
| | ATGGCCACAGTGTCCCACCATCAAAGAGATCAGAGACCAGGGCTCCTGTCGCTCCTGCTGGGTTTCT | | |
| | GGTGGCCTCTATGAATCCCATGTAGGGTGCAGACCGTACTCCATCCCTCCCTGTGAGCACCACGTCA | | |
| | ACGGCTCCCGGCCCCCATGCACGGGGGAGGCAGATACCCCCAAGTGTAGCAAGATCTGTGAGCCTGG | | |
| | CTACAGCCCGACCTACAAACAGGACAAGCACTACGGATACAATTCCTACACCGTCTCCAATAGCGAG | | |
| | AAGGACATCATGGCCGAGATCTACAAAAACGGCCCCGTGGAGGGAGCTTTCTCTGTGTATTCGGACT | | |
| | TCCTGCTCTACAAGTCAGGAGTGTACCAACACGTCACCGAAGAGATGATCGGTGGCCATGCCATCCG | | |
| | CATCCTGGGCTGGGGAGTGGAGAATGGCACACCCTACTGGCTGGTTGCCAACTCCTGGAACACTGAC | | |
| | TGGGGTGACAATGGCTTCTTTAAAATACTCAGAGGACAGGATCACTGTGGAATCGAATCAGAAGTGG | | |
| | TGGCTGGAATTCCACGCACCGATCAGTACTGGGAAAAGATCTAATCTGCCGTGGGCCTGTCGTGCCA | | |
| | GTCCTGGGGGCGAGATCGGGGTA | | |
| | ORF Start: ATG at 137 | | ORF Stop: TAA at 980 |
| | SEQ ID NO: 202 | 281 aa | MW at 31732.5 kD |
| NOV49d, | MWQLWASLCCLLVLANARSRPSFHPLSDELVNYVNRRNTTWQAGHNFYNVDMSYLKRLCGTFLGGPK | | |
| CG56836-04 | | | |
| Protein Sequence | PPQRVMFTEDLKLPASFDAREQWPQCPTIKEIRDQGSCGSCWVSGGLYESHVGCRPYSIPPCEHHVN | | |
| | GSRPPCTGEGDTPKCSKICEPGYSPTYKQDKHYGYNSYSVSNSEKDIMAEIYKNGPVEGAFSVYSDF | | |
| | LLYKSGVYQHVTGEMMGGHAIRILGWGVENGTPYWLVANSWNTDWGDNGFFKILRGQDHCGIESEVV | | |
| | AGIPRTDQYWEKI | | |
| | SEQ ID NO: 203 | 340 bp | |
| NOV49e, | AGGCTCCGCGGCCGCCCCCTTCACCGGATCCCPGCCTGCAAGCTTCGATGCACGGGAACAATGGCCA | | |
| 247856403 DNA | | | |
| Sequence | CAGTGTCCCACCATCAAAGAGATCAGAGACCAGGGCTCCTGTGACTCCTGCTGGGCCTTCGGGGCTG | | |
| | TGGAAGCCATCTCTGACCGGATCTGCATCCACACCAATGCGCACGTCAGCCTCGAGGTCTCGCCGGA | | |
| | GGACCTGCTCACATGCTGTGGCAGCATGTGTGGGGACGGCTGTAATGGTGGCTATCCTGCTGAAGCT | | |
| | TGGAACTTCTGGACAAGAAAAGGCCTGGTTTCTGGTGGCCTCTATCTCGAGGGCAAGGGTGGGCGCG | | |
| | CCGAC | | |
| | ORF Start: at 2 | | ORF Stop: end of sequence |
| | SEQ ID NO: 204 | 113 aa | MW at 11834.0 kD |
| NOV49e, | GSAAAPFTGSLPASFDAREQWPQCPTIKEIRDQGSCGSCWAFGAVEAISDRICIHTNAHVSVEVSAE | | |
| 247856403 | | | |
| Protein Sequence | DLLPCCGSMCGDGCNGGYPAEAWNFWTRKGLVSGGLYLEGKGGRAD | | |
| | SEQ ID NO: 205 | 1376 bp | |
| NOV49f, | AGGCTCCGCGGCCGCCCCCTTCACCGGATCCTCCAATAGCGAGAAGGACATCATGGCCGAGATCTAC | | |
| 247856434 DNA | | | |
| Sequence | AAAAACGGCCCCGTGCAGGGAGCTTTCTCTGTGTATTCGGACTTCCTGCTCTACAAGTCAGGAGTGT | | |
| | ACCAACACGTCACCGGAGAGATGATGGGTGGCCATGCCATCCCCATCCTGGGCTGGGGAGTGGAGAA | | |
| | TGGCACACCCTACTGGCTGGTTGCCAACTCCTGGAACACTGACTGGGGTGACAATGGCTTCTTTAAA | | |
| | ATACTCAGAGGACAGGATCACTGTGGAATCGAATCAGAAGTGGTGGCTGGAATTCCACGCACCGATC | | |
| | AGTACTGGGAAAAGATCCTCGAGGGCAAGGGTGGGCGCGCC | | |
| | ORF Start: at 2 | | ORF Stop: end of sequence |
| | SEQ ID NO: 206 | 125 aa | MW at 13666.1 kD |
| NOV49f, | GSAAAPFTGSSNSEKDIMAEIYKNGPVEGAFSVYSDFLLYKSGVYQHVTGEMMGGHAIRILGWGVEN | | |
| 247856434 | | | |
| Protein Sequence | GTPYWLVANSWNTDWGDNGFFKILRGQDHCGIESEVVAGIPRTDQYWEKILEGKGGRA | | |
| | SEQ ID NO: 207 | 574 bp | |
| NOV49g, | AGGCTCCGCGGCCGCCCCCTTCACCGGATCCATGTGGCAGCTCTGGGCCTCCCTCTGCTGCCTGCTG | | |
| 247856497 DNA | | | |
| Sequence | GTGTTGGCCAATGCCCGGAGCAGGCCCTCTTTCCATCCCCTGTCGGATGAGCTGGTCAACTATGTCA | | |
| | ACAAACGGAATACCACGTGGCAGGCCGGGCACAACTTCTACAACGTGGACATGAGCTACTTGAAGAG | | |
| | GCTATGTGGTACCTTCCTGGGTGGGCCCAAGCCACCCCAGAGAGTTATGTTTACCGAGGACCTGAAG | | |
| | CTGCCTGCAAGCTTCGATGCACGGGAACAATGGCCACAGTGTCCCACCATCAAAGAGATCAGAGACC | | |
| | AGGGCTCCPGTGGCTCCTGCTGGGCCTTCGGGGCTGTGGAAGCCATCTCTGACCGGATCTGCATCCA | | |
| | CACCAATGCGCACGTCAGCGTGGAGGTGTCGGCGGAGGACCTGCTCACATGCTGTGGCAGCATGTGT | | |
| | GGGGACGGCTGTAATGGTGGCTATCCTGCTGAAGCTTGAACTTCTGGAACAAGAAAAGGCCTGGTTT | | |
| | CTGGTGGCCTCTATCTCGAGGGCAAGGGTGGGCGCGCC | | |
| | ORF Start: at 2 | | ORF Stop: end of sequence |
| | SEQ ID NO: 208 | 191 aa | MW at 20877.5 kD |
| NOV49g, | GSAAAPFTGSMWQLWASLCCLLVLANARSRPSFHPLSDELVNYVNKRNTTWQAGHNFYNVDMSYLKR | | |
| 247856497 | | | |
| Protein Sequence | LCGTGLGGPKPRQRVMFTEDLKLPASFDAREQWPQCPTIKEIRDQGSCGSCWAFGAVEAISDRICIH | | |
| | TNAHVSVEVSAEDLLTCCGSMCGDGCNGGYPAEAWNFWTRKGLVSGGLYLEGKGGRA | | |
| | SEQ ID NO: 209 | 590 bp | |
| NOV49h, | AGGCTCCGCGGCCGCCCCCTTCACCGGATCCATGTGGCAGCTCTGGGCCTCCCTCTGCTGCCTGCTG | | |
| 247856493 DNA | | | |
| Sequence | GTGTTGGCCAATGCCCGGAGCAGGCCCTCTTTCCATCCCGTGTCGGATGAGCTGGTCAACTATGTCA | | |
| | ACAAACGGAATACCACGTGGCAGGCCGGGCACAACTTCTACAACGTGGACATGCGCTACTTCAAGAG | | |
| | GCTATGTGGTACCTTCCTCGGTCGGCCCAAGCCACCCCAGAGAGTTATGTTTACCGAGGACCTGAAG | | |
| | CTGCCTGCAAGCTTCGATGCACGGGAACAATGGCCACAGTGTCCCACCATCAAAGAGATCAGAGACC | | |
| | AGGGCTCCTGTGGCTCCTGCTGGGCCTTCGGCGCTGTGGAAGCCATCTCTGACCGGATCTGCATCCA | | |
| | CACCAATGCGCACGTCAGCGTGGAGGTGTCGGCGGAGGACCTGCTCACCTGCTGTGGCAGCATGTGT | | |
| | GGGGACGGCTGTAATGGTGGCTATCCTCCTGAAGCTTGGAACTTCTGGACAAGAAAAGGCCTGGTTT | | |
| | CTGGTGGCCTCTATCTCGAGGGCAAGGGTGGGCGCGCCGACCCAGCTTTCTGTA | | |
| | ORF Start: at 2 | | ORF Stop: end of sequence |
| | SEQ ID NO: 210 | 197 aa | MW at 21367.0 kD |
| NOV49h, | GSAAAPFTGSMWQLWASLCCLLVLANARSRPSFHPVSDELVNYVNKRNTTWQAGHNFYNVDMGYLKR | | |
| 247856493 | | | |
| Protein Sequence | LCGTFLGGPKPPQRVMFTEDLKLPASFDAREQWPQCPTIKEIRDQGSCGSCWAFGAVEAISDRICIH | | |
| | TNAHVSVEVSAEDLLTCCGSMCGDGCNGGYPAEAWNFWTRKGLVSGGLYLEGKGGRADPAFCX | | |
| | SEQ ID NO: 211 | 551 bp | |
| NOV49i, | AGGCTCCGCGGCCGCCCCCTTCACCGGATCCCGGAGCAGGCCCTCTTTCCATCCCCTGTCGGATGAG | | |
| 247856574 DNA | | | |
| Sequence | CTGGTCAACTATGTCAACAAACGGAATACCACGTGGCAGGCCGCGCACAACTTCTACAACGTGGACA | | |
| | TGACCTACTTGAAGAGGCTATGTGGTACCTTCCTGGGTGGGCCCAAGCCACCCCAGAGAGTTATGTT | | |
| | TACCGAGGACCTGAAGCTGCCTGCAAGCTTCGATGCACGGGAACAATGGCCACAGTGTCCCACCATC | | |
| | AAAGAGATCAGAGACCAGGGCTCCTGTGGCTCCTGCTCGGCCTTCGGCGCTGTGGAAGCCATCTCTG | | |
| | ACCGGATCTGCATCCACACCAATGCGCACGTCAGCGTGGAGGTGTCGGCGGAGGACCTGCTCACATG | | |
| | CTGTGGCAGCATGTGTGGAGACGGCTGTAATGGTGGCTATCCTGCTGAAGCTTCGAACTTCTGGACA | | |
| | AGAAAAGGCCTGGTTTCTCGTGGCCTCTATCTCGAGGGCAAGGGTGGGCGCCCCGACCCAGCTTTCC | | |
| | CGTACAAAGCTGGCA | | |
| | ORF Start: at 2 | | ORF Stop: end of sequence |
| | SEQ ID NO: 212 | 184 aa | MW at 19933.2 kD |
| NOV49i, | GSAAAPFTGSRSRPSFHPLSDELVNYVNKRNTTWQAGHNFYNVDMSYLKRLCGTFLGGPKPPQRVMF | | |
| 247856574 | | | |
| Protein | TEDLKLPASFDAREQWPQCPTIKEIRDQGSCCSCWAFGAVEAISDRICIHTNAHVSVEVSAEDLLTC | | |
| Sequence | | | |
| | CGSMCGDGCNGGYPAEAWNFWTRKGLVSGGLYLEGKGGRPDPAFPYKAGX | | |
| | SEQ ID NO: 213 | 523 bp | |
| NOV49j, | AGGCTCCGCGGCCGCCCCCTTCACCGGATCCCGGAGCAGGCCCTCTTTCCATCCCCTGTCGGATCAG | | |
| 247856545 DNA | | | |
| Sequence | CTGGTCAACTATGTCAACAAACGGAATACCACGTGGCAGGCCGGGCACAACTTCTACAACGTGGACA | | |
| | TGAGCTACTTGAAGAGGCTATGTGGTACCTTCCTGGGTGGGCCCAAGCCACCCCTGAGACTTATGTT | | |
| | TACCGACGACCTGAAGCTGCCTGCAAGCTTCGATGCACGGGAACAATGGCCACAGTGTCCCACCATC | | |
| | AAAGAGATCAGAGACCAGGGCTCCTGTGGCTCCTGCTGGGCCTTCGGGGCTGTCGAAGCCATCTCTG | | |
| | ACCGGATCTGCATCCACACCAATGCGCACGTCAGCGTGGAGGTGTCGGCCCAGGACCTGCTCACATG | | |
| | CTGTGGCGGCATGTGTGGGGACGCCTGTAATGGTGGCTATCCTGCTGAAGCTTGGAACTTCTGGACA | | |
| | AGAAAAGGCCTGGTTTCTGGTGGCCTCTATCTCGAGGGCAAGGGTGGGCGCGCC | | |
| | ORF Start: at 2 | | ORF Stop: end of sequence |
| | SEQ ID NO: 214 | 174 aa | MW at 18915.1 kD |
| NOV49j, | GSAAAPFTGSRSRPSFHPLSDELVNYVNKRNTTWQAGHNFYNVDMSYLKRLCGTFLGGPKPPLRVMF | | |
| 247856545 | | | |
| Protien | TEDLKLPASFDAREQWPQCPTIKEIRDQCSCGSCWAFGAVEAISDRICIHTNAHVSVEVSAEDLLTC | | |
| Sequence | | | |
| | CGGMCCDCCNGGYPAEAWNFWTRKGLVSGGLYLEGKGGRA | | |
| | SEQ ID NO:215 | 1036 bp | |
| NOV49k, | CACCCTCGAGATGTGGCAGCTCTGGGCCTCCCTCTGCTGCCTGCTGGTGTTGGCCAATGCCCGGACC | | |
| 275480714 DNA | | | |
| Sequence | AGGCCCTCTTTCCATCCCCTGTCGGATGACCTGGTCAACTATGTCAACAAACGCAATACCACGTGGC | | |
| | AGGCCGGGCACAACTTCTACAACGTGGACATGAGCTACTTGAAGAGGCTATGTGGTACCTTCCTGGG | | |
| | TGGGCCCAAGCCACCCCAGAGAGTTATGTTTACCGAGGACCTGAAGCTGCCTGCAAGCTTCGATGCA | | |
| | CGGGAACAATGGCCACAGTGTCCCACCATCAAAGAGATCAGAGACCAGGGCTCCTGTGGCTCCTGCT | | |
| | GGGCCTTCGGGGCTGTGGAAGCCATCTCTGACCGGATCTGCATCCACACCAATGCGCACGTCAGCGT | | |
| | GGAGGTGTCGGCGGAGGACCTGCTCACATGCTGTGGCAGCATGTGTGGGGACGGCTGTAATGGTGGC | | |
| | TATCCTGCTGAAGCTTGGAACTTCTGGACAAGAAAAGGCCTGGTTTCTGGTGGCCTCTATGAATCCC | | |
| | ATGTAGGGTGCAGACCGTACTCCATCCCTCCCTGTGAGCACCACGTCAACGGCTCCCGGCCCCCATC | | |
| | CACGGGGGAGGGAGATACCCCCAAGTGTAGCAAGATCTGTGAGCCTGGCTACAGCCCGACCTACAAA | | |
| | CAGGACAAGCACTACGGATACAATTCCTACAGCGTCTCCAATAGCGAGAAGGACATCATGGCCGAGA | | |
| | TCTACAAAAACCGCCCCGTGGAGGCAGCTTTCTCTGTGTATTCGGACTTCCTGCTCTACAAGTCAGG | | |
| | AGTGTACCAACACGTCACCGGAGAGATGATGGGTGGCCATGCCATCCGCATCCTGGGCTGGGGAGTG | | |
| | GAGAATGGCACACCCTACTGGCTGGTTGCCAACTCCTGGAACACTGACTGGGGTGACAATGCCTTCT | | |
| | TTAAAATACTCAGAGGACAGGATCACTGTGGAATCGAATCAGAAGTGGTGGCTGGAATTCCACGCAC | | |
| | CGATCAGTACTGGCAAAAGATCGTCGACGGC | | |
| | ORF Start: at 2 | | ORF Stop: end of sequence |
| | SEQ ID NO: 216 | 345 aa | MW at 38435.9 kD |

| | |
|---|---|
| NOV49k, | TLEMWQLWASLCCLLVLANARSRPSFHPLSDELVNYVNKRNTTWQAGHNFYWVDMSYLKRLCGTFLC |
| 275480714 | |
| Protein Sequence | GPKPPQRVMFTEDLKLPASFDAREQWPQCPTIKEIRDQGSCGSCWAFGAVEAISDRICIHTNAHVSV |
| | EVSAEDLLTCCGSMCGDGCNGGYPAEAWNFWTRKGLVSGGLYESHVGCRPYSIPPCEHHVNGSRPPC |
| | TGEGDTPKCSKICEPGYSPTYKQDKHYGYNSYSVSNSEKDIMAEIYKNGPVEGAFSVYSDFLLYKSG |
| | VYQHVTGEMMCCHAIRILGWGVENGTPYWLVANSWNTDWGDNGFFKILRGQDHCGIESEVVAGIPRT |
| | DQYWEKIVDG |

Sequence comparison of the above protein sequences yields the following sequence relationships shown in Table 49B.

TABLE 49B
Comparison of NOV49a against NOV49b through NOV49k.

| Protein | NOV49a Residues/ | Identities/ |
|---|---|---|
| Sequence | Match Residues | Similarities for the Matched Region |
| NOV49b | 1 . . . 141 | 141/141 (100%) |
| | 1 . . . 141 | 141/141 (100%) |
| NOV49c | 1 . . . 176 | 175/176 (99%) |
| | 1 . . . 176 | 176/176 (99%) |
| NOV49d | 1 . . . 339 | 279/339 (82%) |
| | 1 . . . 281 | 280/339 (82%) |
| NOV49e | 80 . . . 180 | 96/101 (95%) |
| | 11 . . . 111 | 96/101 (95%) |
| NOV49f | 233 . . . 339 | 107/107 (100%) |
| | 11 . . . 117 | 107/107 (100%) |
| NOV49g | 1 . . . 180 | 175/180 (97%) |
| | 11 . . . 190 | 175/180 (97%) |
| NOV49h | 1 . . . 180 | 173/180 (96%) |
| | 11 . . . 190 | 174/180 (96%) |
| NOV49i | 17 . . . 181 | 159/165 (96%) |
| | 10 . . . 174 | 160/165 (96%) |
| NOV49j | 17 . . . 180 | 144/164 (87%) |
| | 10 . . . 173 | 145/164 (87%) |
| NOV49k | 1 . . . 339 | 339/339 (100%) |
| | 4 . . . 342 | 339/339 (100%) |

Further analysis of the NOV49a protein yielded the following properties shown in Table 49C.

TABLE 49C
Protein Sequence Properties NOV49a

| | |
|---|---|
| PSort analysis: | 0.3700 probability located in outside; |
| | 0.1900 probability located in lysosome |
| | (lumen); 0.1376 probability located in |
| | microbody (peroxisome); 0.1000 probability |
| | located in endoplasmic reticulum (membrane) |
| SignalP analysis: | Cleavage site between residues 18 and 19 |

A search of the NOV49a protein against the Geneseq database, a proprietary database that contains sequences published in patents and patent publication, yielded several homologous proteins shown in Table 49D.

TABLE 49D
Geneseq Results for NOV49a

| | | | Identities/ | |
|---|---|---|---|---|
| | | NOV49a | Similarities for | |
| Geneseq | Protein/Organism/Length | Residues/Match | the Matched | Expect |
| Identifier | [Patent #, Date] | Residues | Region | Value |
| AAR90616 | Anti-procathepsin B | 1 . . . 339 | 338/339 (99%) | 0.0 |
| | monoclonal antibody - Homo | 1 . . . 339 | 339/339 (99%) | |
| | sapiens, 339 aa. | | | |
| | [JP07309900-A, | | | |
| | 28-NOV-1995] | | | |
| AAB53470 | Human colon cancer antigen | 1 . . . 339 | 338/339 (99%) | 0.0 |
| | protein sequence SEQ ID: | 6 . . . 344 | 338/339 (99%) | |
| | NO: 1010 - Homo sapiens, | | | |
| | 344 aa. [WO200055351-A1, | | | |
| | 21-SEP-2000] | | | |
| ABP41147 | Human ovarian antigen | 1 . . . 339 | 290/339 (85%) | 0.0 |
| | HOFMP73, SEQ ID: | 8 . . . 346 | 317/339 (92%) | |
| | NO: 2279 - Homo sapiens, | | | |
| | 346 aa. [WO200200677-A1, | | | |
| | 03-JAN-2002] | | | |
| ABB06116 | Human NS protein sequence | 1 . . . 267 | 266/267 (99%) | e−167 |
| | SEQ ID NO: 208 - Homo | 1 . . . 267 | 266/267 (99%) | |
| | sapiens, 273 aa. | | | |
| | [WO200206315-A2, | | | |
| | 24-JAN-2002] | | | |
| ABB65378 | Drosophila melanogaster | 13 . . . 331 | 190/330 (57%) | e−113 |
| | polypeptide SEQ ID NO: | 13 . . . 339 | 232/330 (69%) | |
| | 22926 - Drosophila | | | |
| | melanogaster, 340 aa. | | | |
| | [WO200171042-A2, | | | |
| | 27-SEP-2001] | | | |

In a BLAST search of public sequence datbases, the NOV49a protein was found to have homology to the proteins shown in the BLASTP data in Table 49E.

TABLE 49E
Public BLASTP Results for NOV49a

| | | | Identities/ | |
|---|---|---|---|---|
| Protein | | NOV49a | Similarities for | |
| Accession | | Residues/Match | the Matched | Expect |
| Number | Protein/Organism/Length | Residues | Portion | Value |
| P07858 | Cathepsin B precursor (EC | 1 . . . 339 | 338/339 (99%) | 0.0 |
| | 3.4.22.1) (Cathepsin B1) (APP | 1 . . . 339 | 339/339 (99%) | |
| | secretase) - Homo sapiens | | | |
| | (Human), 339 aa. | | | |
| KHBOB | cathepsin B (EC 3.4.22.1) | 1 . . . 335 | 280/335 (83%) | e−180 |
| | precursor - bovine, 335 aa. | 1 . . . 335 | 307/335 (91%) | |
| P07688 | Cathepsin B precursor (EC | 1 . . . 335 | 279/335 (83%) | e−180 |
| | 3.4.22.1) - Bos taurus | 1 . . . 335 | 307/335 (91%) | |
| | (Bovine), 335 aa. | | | |
| P00787 | Cathepsin B precursor (EC | 1 . . . 336 | 265/336 (78%) | e−175 |
| | 3.4.22.1) (Cathepsin B1) | 1 . . . 336 | 299/336 (88%) | |
| | (RSG-2) - Rattus norvegicus | | | |
| | (Rat), 339 aa. | | | |
| P10605 | Cathepsin B precursor (EC | 1 . . . 336 | 267/336 (79%) | e−174 |
| | 3.4.22.1) (Cathepsin B1) - | 1 . . . 336 | 297/336 (87%) | |
| | Mus musculus (Mouse), 339 | | | |
| | aa. | | | |

PFam analysis predicts that the NOV49a protein contains the domains shown in the Table 49F.

TABLE 49F
Domain Analysis of NOV49a

| | | Identities/ | |
|---|---|---|---|
| Pfam | NOV49a | Similarities | Expect |
| Domain | Match Region | for the Matched Region | Value |
| Peptidase_C1 | 80 . . . 329 | 112/344 (33%) | 1.3e−117 |
| | | 218/344 (63%) | |

### Example 50

The NOV50 clone was analyzed, and the nucleotide and encoded polypeptide sequences are shown in Table 50A.

TABLE 50A
NOV50 Sequence Analysis

| | SEQ ID NO: 217 | 960 bp | |
|---|---|---|---|
| NOV50a, | CCCGTCCGAGCCCCGGCCCCAAGTAACGCCGCCGCCCCGGAGCCGCCTTGGAGGTCCCCCTCCCCAC | | |
| CG572840-01 | | | |
| DNA Sequence | TAAGTGCCTCTTTGCATACCACCAGTCCCCACCCGCACGCTCTCTGGACCACTACAGCTGGACGGGC | | |
| | AATGGCGGGTCGGGGAGGCGCACGACGACCCAATGGACCAGCTGCTGGGAACAAGATCTGTCAATTT | | |
| | AACCTGGTTCTGCTGGGGGAGTCTGCGGTAGGCAAATCCAGCCTCGTCCTCCGCTTTGTCAAGGGAC | | |
| | AGTTTCACGAGTACCAGGAGAGCACAATTGGAGCGGCCTTCCTCACACAGACTGTCTGCCTGGATGA | | |
| | CACAACAGTCAAGTTTGAGATCTGGGACACAGCTGGACAGGAGCGGTATCACAGCCTGGCCCCCATG | | |
| | TACTATCCGCGGGCCCAGGCTGCCATCGTGGTCTATGACATCACCAACACAGATACATTTGCACGGG | | |
| | CCAAGAACTGGGTGAAGGAGCTACAGAGCCAGGCCAGCCCCAACATCGTCATTGCACTCGCGGGTAA | | |
| | CAAGGCAGACCTGGCCACCAAGAGAGCCGTGGAATTCCAGGAAGCACAAGCCTATGCAGACGACAAC | | |
| | AGTTTGCTGTTCATGGAGACATCAGCAAAGACTGCAATGAACGTGAACGAAATCTTCATGGCAATAG | | |
| | CTAAGAAGCTTCCCAAGAACGAGCCCCAGAATGCAACTGGTGCTCCAGGCCGAAACCGAGGTGTGGA | | |
| | CCTCCAGGAGAACAACCCAGCCAGCCGGAGCCAGTGCTGCACCAACTGAGCCCCCCTTGCCTGCCCG | | |
| | CTGCCCCCGCCTCCTCCGCCTGAATGACCCGACTGGAATCCACTCTAACCAATCGCACTTAACGACT | | |
| | CGGGCCACCACTGGGGGGGCAGGGGGAGGGGTCCACCATGATTTCTCCATATAATTTTGATCATAGG | | |
| | CCGGAGTGAGTCATTCCACCTG | | |
| | ORF Start: ATG at 136 | | ORF Stop: TGA at 784 |
| | SEQ ID NO: 218 | 216 aa | MW at 23567.4 kD |
| NOV50a, | MAGRGGARRPNGPAAGNKICQFKLVLLGESAVGKSSLVLRFVKGQFHEYQESTIGAAFLTQTVCLDD | | |
| CG57284-01 | | | |
| Protein Sequence | TTVKFEIWDTAGQERYHSLAPMYYRGAQAAIVVYDITNTDTFARAKNWVKELQRQASPNIVIALAGN | | |
| | KADLASKRAVEFQEAQAYADDNSLLFMETSAKTANNVNEIFMATAXKLPKNEPQNATGAPGRNRGVD | | |
| | LQENNPASRSQCCSN | | |
| | SEQ ID NO: 219 | 747 bp | |
| NOV50b, | CCACTAAGTGCCTCTTTGCATAGCACCAGTCCCCACCCGCACGCTCTCTGGACCACTACAGCTGGAC | | |
| CG57284-03 | | | |
| DNA Sequence | GGGCAATGGCGGGTCGCGGAGGCGCAGCACGACCCAATGGACCAGCTGCTGGGAACAAGATCTGTCA | | |
| | ATTTAAGCTGGTTCTGCTGGGGGAGTCTGCGGTAGGCAAATCCAGCCTCGTCCTCCGCTTTGTCAAG | | |
| | GGACAGTTTCACGAGTACCAGGAGAGCACAATTGGAGCGGCCTTCCTCACACAGACTGTCTGCCTCG | | |
| | ATGACACAACAGTCAAGTTTGAGATCTGGGACACAGCTGGACAGGACCGGTATCACAGCCTGGCCCC | | |
| | CATGTACTATCGGGCGGCCCAGGCTGCCATCGTGGTCTATGACATCACCAACATCGTCATTGCGCTC | | |
| | GCGGGTAACAAGGCAGACCTGGCCAGCAAGAGAGCCGTCGAATTCCAGGAAGCACAAGCCTATGCAG | | |
| | ACGACAACACTTTGCTGTTCATGGAGACATCAGCAAAGACTGCAATGAACGTGAACGAAATCTTCAT | | |
| | GGCAATAGCTAAGAAGCTTCCCAAGAACGAGCCCCAGAATGCAACTCGTGCTCCACGCCGAAACCGA | | |
| | GGTGTGGACCTCCAGGAGAACAACCCAGCCAGCCGGAGCCACTGCTGCAGCAACTGAGCCCCCCTTG | | |
| | CCTGCCCGCTGCCCCCGCCTCCTCCGCCTCAATGACCCCACTGGAATCCACTCTAACCAATCGCACT | | |
| | TAACGACTCG | | |
| | ORF Start: ATG at 73 | | ORF Stop: TGA at 658 |
| | SEQ ID NO: 220 | 195 aa | MW at 21039.6 kD |
| NOV50b, | MAGRGGAARPNGPAAGNKICQFKLVLLGESAVGKSSLVLRFVKGQFHEYQESTIGAAFLTQTVCLDD | | |
| CG57285-03 | TTVKFEIWDTAGQERYHSLAPMYYRGAQAAIVVYDITNIVIALAGNKADLASKRAVEFQEAQAYADD | | |
| Protein Sequence | NSLLFMETSAKTAMNVNEIFMAIAKKLPKNEPQNATGAPGRNRGVDLQENNPASRSQCCSN | | |
| | SEQ ID NO: 221 | 819 bp | |
| NOV50c, | AATCGCCTTCCACTAAGTGCCTCTTTGCATAGCACCAGTCCCCACCCGCACGCTCTCTGGACCACTA | | |
| CG57284-02 | CAGCTGGACGGGCAATGGCGGGTCGGGGAGGCGCACCACGACCCAATGGACCAGCTGCTGGGAACAA | | |
| DNA Sequence | GATCTGTCAATTTAAGCTGGTTCTGCTGGGGGAGTCTGCGGTAGGCAAATCCAGCCTCGTCCTCCGC | | |
| | TTTGTCAAGGGACAGTTTCACGAGTACCAGGAGAGCACAATTGGAGCGGCCTTCCTCACACAGACTC | | |
| | TCTGCCTGGATGACACAACAGTCAAGTTTGAGATCTGGGACACAGCTGGACAGGAGCGGTATCACAG | | |
| | CCTGGCCCCCATGTACTATCGGGGGGCCCAGGCTGCCATCGTGGTCTATGACATCACCAACACAGAT | | |
| | ACATTTGCACGCGCCAAGAACTGGGTGAAGGAGCTACAGAGGCAGGCCAGCCCCAACATCGTCATTG | | |
| | CACTCGCGGGTAACAAGGCAGACCTGGCCAGCAAGAGAGCCGTGGAATTCCAGGAAGCACAAGCCTA | | |
| | TGCAGACGACAACAGTTTGCTGTTCATGGAGACATCAGCAAAGACTGCAATGAACGTGAACGAAATC | | |
| | TTCATGGCAATAGCTAACAAGCTTCCCAAGAACGAGCCCCAGAATGCAACTGGTGCTCCAGGCCGAA | | |
| | ACCGAGGTGTGGACCTCCAGGAGAACAACCCAGCCAGCCGGAGCCAGTGCTGCAGCAACTGAGCCCC | | |
| | CCTTGCCTGCCCCCTGCCCCCGCCTCCTCCGCCTGAATGACCCGACTGGAATCCACTCTAACCAATC | | |
| | GCACTTAACGACTCG | | |
| | ORF Start: ATG at 82 | | ORF Stop: TGA at 730 |
| | SEQ ID NO: 222 | 216 aa | MW at 23482.3 kD |

| | |
|---|---|
| NOV50c, | MAGRGGAARPNGPAAGNXICQFKLVLLGESAVGKSSLVLRFVKGQEEEYQESTIGAAFLTQTVCLDD |
| CG57284-02 | TTVKFEIWDTAGQERYHSLAPMYYRGAQAAIVVYDITNTDTFARAKNWVKELQRQASPNIVIALAGN |
| Protein Sequence | KADLASKRAVEFQEAQAYADDNSLLFMETSAKTAMNVNEIFMAIAKKLPKNEPQNATGAPGRNRGVD |
| | LQENNPASRSQCCSN |

Sequence comparison of the above protein sequences yields the following sequence relationships shown in Table 50B.

TABLE 50B
Comparison of NOV50a against NOV50b and NOV50c.

| | NOV50a Residues/ | Identities/Similarities |
|---|---|---|
| Protein Sequence | Match Residues | for the Matched Region |
| NOV50b | 18 . . . 216 | 178/199 (89%) |
| | 18 . . . 195 | 178/199 (89%) |
| NOV50c | 18 . . . 216 | 199/199 (100%) |
| | 18 . . . 216 | 199/199 (100%) |

Further analysis of the NOV50a protein yielded the following properties shown in Table 50C.

TABLE 50C
Protein Sequence Properties NOV50a

| | |
|---|---|
| PSort analysis: | 0.6500 probability located in cytoplasm; 0.2189 |
| | probability located in lysosome (lumen); 0.1000 |
| | probability located in mitochondrial matrix space; |
| | 0.0000 probability located in endoplasmic reticulum |
| | (membrane) |
| SignalP analysis: | No Known Signal Sequence Predicted |

A search of the NOV50a protein against the Geneseq database, a proprietary database that contains sequences published in patents and patent publication, yielded several homologous proteins shown in Table 50D.

TABLE 50D
Geneseq Results for NOV50a

| | | | Identities/ | |
|---|---|---|---|---|
| | | NOV50a | Similarities | |
| | Protein/Organism/ | Residues/ | for the | |
| Geneseq | Length | Match | Matched | Expect |
| Identifier | [Patent #, Date] | Residues | Region | Value |
| AAM79225 | Human protein SEQ | 9 . . . 216 | 179/208 | e−101 |
| | ID NO 1887 - Homo | | (86%) | |
| | sapiens, 215 aa. | 8 . . . 215 | 194/208 | |
| | [WO200157190-A2, | | (93%) | |
| | 09-AUG-2001] | | | |
| AAY56173 | Human Wnt-1 | 9 . . . 216 | 179/208 | e−101 |
| | amino acid | | (86%) | |
| | sequence - Homo | 8 . . . 215 | 194/208 | |
| | sapiens, 215 aa. | | (93%) | |
| | [CA2200794-A, | | | |
| | 24-SEP-1998] | | | |
| AAB28187 | Human RAS-relates | 1 . . . 197 | 178/197 | 9e−97 |
| | protein RAB-5A - | | (90%) | |
| | Homo sapiens, | 1 . . . 192 | 186/197 | |
| | 193 aa. | | (94%) | |
| | [WO200052165-A2, | | | |
| | 08-SEP-2000] | | | |
| AAM80209 | Human protein SEQ | 9 . . . 216 | 172/209 | 1e−95 |
| | ID NO 3855 - Homo | | (82%) | |
| | sapiens, 255 aa. | 47 . . . 255 | 189/209 | |
| | [WO200157190-A2, | | (90%) | |
| | 09-AUG-2001] | | | |
| ABB60036 | Drosophila | 2 . . . 214 | 159/213 | 8e−85 |
| | melanogaster | | (74%) | |
| | polypeptide SEQ | 11 . . . 218 | 177/213 | |
| | ID NO 6900 - | | (82%) | |
| | Drosophila | | | |
| | melanogaster, | | | |
| | 219 aa. | | | |
| | [WO200171042-A2, | | | |
| | 27-SEP-2001] | | | |

In a BLAST search of public sequence datbases, the NOV50a protein was found to have homology to the proteins shown in the BLASTP data in Table 50E.

TABLE 50E
Public BLASTP Results for NOV50a

| | | | Identities/ | |
|---|---|---|---|---|
| | | NOV50a | Similarities | |
| Protein | | Residues/ | for the | |
| Accession | Protein/Organism/ | Match | Matched | Expect |
| Number | Length | Residues | Portion | Value |
| P51148 | Ras-related protein | 1 . . . 216 | 216/216 | e−122 |
| | Rab-5C (RAB5L) | | (100%) | |
| | (L1880) - Homo | 1 . . . 216 | 216/216 | |
| | sapiens (Human), | | (100%) | |
| | 216 aa. | | | |
| AAM21086 | Small GTP binding | 1 . . . 216 | 215/216 | e−121 |
| | protein RAB5C - | | (99%) | |
| | Homo sapiens | 1 . . . 216 | 215/216 | |
| | (Human), 216 aa. | | (99%) | |
| Q8R1V8 | Hypothetical | 1 . . . 216 | 212/216 | e−119 |
| | 23.4 kDa protein - | | (98%) | |
| | Mus musculus | 1 . . . 216 | 213/216 | |
| | (Mouse), 216 aa. | | (98%) | |
| P51147 | Ras-related protein | 1 . . . 216 | 212/216 | e−119 |
| | Rab-5C - | | (98%) | |
| | Canis familiaris | 1 . . . 216 | 213/216 | |
| | (Dog), 216 aa. | | (98%) | |
| Q98932 | Rab5C-like protein - | 1 . . . 216 | 203/216 | e−114 |
| | Gallus gallus | | (93%) | |
| | (Chicken), 216 aa. | 1 . . . 216 | 208/216 | |
| | | | (95%) | |

PFam analysis predicts that the NOV50a protein contains the domains shown in the Table 50F.

TABLE 50F
Domain Analysis of NOV50a

| | | Identities/ | |
|---|---|---|---|
| | NOV50a Match | Similarities for | |
| Pfam Domain | Region | the Matched Region | Expect Value |
| arf | 4 . . . 185 | 40/198 (20%) | 0.0018 |
| | | 105/198 (53%) | |
| ras | 23 . . . 216 | 90/209 (43%) | 3.1e−104 |
| | | 181/209 (87%) | |

### Example 51

The NOV51 clone was analyzed, and the nucleotide and encoded polypeptide sequences are shown in Table 51A.

TABLE 51A
NOV51 Sequence Analysis

| | SEQ ID NO: 223 | 4826 bp | |
|---|---|---|---|
| NOV51a, | AGCTGAGCCCGAGCCCAGACCGCGCCCGCGCCGCCATGCCCCTGGCCTTCTGCGGCAGCGAGAACCA | | |
| CG57308-01 | | | |
| DNA Sequence | GTGCCGCACGTCTTCCTACTCTTCATCACCTTCCCCATCCTCTTCATTGGATGGGGAAGTCAGAGCT | | |
| | CCAAGGTGCACATCCACCACACCACATGGCTTCATTTCCCTGGCCACAACCTGCGGTGGATCCTGAC | | |
| | CTTCATGCTGCTCTTCGTCCTGGTGTGTGAGATTGCAGAGGGCATCCTGTCTGATGGGGTGACCGAA | | |
| | TCCCACCATCTGCACCTGTACATGCCAGCCGGGATGGCGTTCATGGCTGCTGTCACCTCCGTGGTCT | | |
| | ACTATCACAACATCGAGACTTCCAACTTCCCCAAGCTGCTAATTGCCCTGCTGGTGTATTGGACCCT | | |
| | GGCCTTCATCACCAAGACCATCAAGTTTGTCAAGTTCTTGGACCACGCCATCGGCTTCTCGCAGCTA | | |
| | CGCTTCTGCCTCACAGGGCTGCTGGTGATCCTCTATGGGATGCTGCTCCTCGTGGAGGTCAATGTCA | | |
| | TCAGGGTGACGAGATACATCTTCTTCAAGACACCGAGGGAGGTGAAGCCTCCCGAGGACCTGCAAGA | | |
| | CCTGGGGGTACGCTTCCTGCAGCCCTTCGTGAATCTGCTGTCCAAAGGCACCTACTGGTGGATGAAC | | |
| | GCCTTCATCAAGACTGCCCACAAGAAGCCCATCGACTTGCGAGCCATCGGGAAGCTGCCCATCGCCA | | |
| | TCAGGGCCCTCACCAACTACCAACGGCTCTGCGAGGCCTTTGACGCCCAGGTGCGGAAGGACATTCA | | |
| | GGGCACTCAAGGTGCCCGGGCCATCTGGCAGGCACTCAGCCATGCCTTCGGGAGCCGCCTGGTCCTC | | |
| | AGCAGCACTTTCCGCATCTTGGCCGACCTCCTGGGCTTCGCCGGGCCACTGTGCATCTTTGGGATCG | | |
| | TGGACCACCTTGGGAAGGAGAACCACGTCTTCCAGCCCAAGACACAATTTCTCGGGGTTTACTTTGT | | |
| | CTCATCCCAAGAGTTCCTTGCCAATGCCTACGTCTTAGCTGTGCTTCTGTTCCTTGCCCTCCTACTG | | |
| | CAAAGGACATTTCTGCAACCATCCTACTATGTGGCCATTGAAACTGGAATTAACTTGAGACGAGCAA | | |
| | TACAGACCAAGATTTACAATAAAATTATGCACCTGTCCACCTCCAACCTGTCCATGGGAGAAATGAC | | |
| | TGCTGGACAGATCTGTAATCTGGTTGCCATCGACACCAATCAGCTCATGTGGTTTTTCTTCTTGTGC | | |
| | CCAAACCTCTGGGCTATGCCAGTACAGATCATPGTGGGTGTGATTCTCCTCTACTACATACTCGGAG | | |
| | TCAGTGCCTTAATTGGAGCAGCTGTCATCATTCTACTGGCTCCTGTCCAGTACTTCGTGGCCACCAA | | |
| | GCTGTCTCAGGCCCAGCGCAGCACACTGGAGTATTCCAATGAGCGGCTGAAGCAGACCAACGAGATG | | |
| | CTCCGCGGCATCAAGCTGCTGAAGCTGTACGCCTGGGAGAACATCTTCCCCACGCGGGTGGAGACGA | | |
| | CCCGCAGGAAGGAGATGACCAGCCTCACGGCCTTTGCCATCTATACCTCCATCTCCATTTTCATGAA | | |
| | CACGGCCATCCCCATTGCAGCTGTCCTCATAACTTTCGTGGGCCATGTCAGCTTCTTCAAAGAGGCC | | |
| | GACTTCTCGCCCTCCGTGGCCTTTGCCTCCCTCTCCCTCTTCCATATCTTGGTCACACCGCTGTTCC | | |
| | TGCTGTCCAGTGTGGTCCGATCTACCGTCAAAGCTCTAGTGAGCGTGCAAAACCTAAGCGAGTTCCT | | |
| | GTCCAGTGCAGAGATCCGTGAGGAGCAGTGTGCCCCCCATGAGCCCACACCTCAGGGCCCAGCCAGC | | |
| | AAGTACCAGGCGGTGCCCCTCAGGGTTGTGAACCGCAAGCGTCCAGCCCGGGAGGATTGTCGGGGCC | | |
| | TCACCGGCCCACTGCAGAGCCTGGTCCCCAGTGCAGATGGCGATGCTGACAACTGCTGTGTCCAGAT | | |
| | CATGGGACGCTACTTCACGTGGACCCCAGATGGAATCCCCACACTGTCCAACATCACCATTCGTATC | | |
| | CCCCGAGGCCAGCTGACTATGATCGTGGGGCACGTGGGCTGCGGCAAGTCCTCGCTCCTTCTAGCCG | | |
| | CACTGGGGGAGATGCAGAAGGTCTCAGGGGCTGTCTTCTGGAGCAGCCTTCCTGACAGCGAGATAGG | | |
| | AGAGGACCCCAGCCCAGAGCGGGAGACAGCGACCGACTTGGATATCAGGAAGAGAGGCCCCGTGGCC | | |
| | TATGCTTCGCAGAAACCATGGCTGCTAAATGCCACTGTGGAGGAGAACATCATCTTTGAGAGTCCCT | | |
| | TCAACAAACAACGGTACAAGATGGTCATTGAAGCCTGCTCTCTGCAGCCAGACATCGACATCCTGCC | | |
| | CCATGGAGACCAGACCCAGATTGGGGAACGGGGCATCAACCTGTCTCGTGGTCAACGCCAGCGAATC | | |
| | AGTGTGGCCCGAGCCCTCTACCACCACGCCAACGTTGTCTTCTTGGATGACCCCTTCTCAGCTCTGG | | |
| | ATATCCATCTGAGTGACCACTTAATGCAGGCCGGCATCCTTGAGCTGCTCCGGGACGACAAGAGGAC | | |
| | AGTGGTCTTAGTGACCCACAAGCTACAGTACCTGCCCCATCCAGACTGGATCATTGCCATGAAGGAT | | |
| | GGCACCATCCAGAGGGAGGTACCCTCAAGAGACTTCCAGAGGTCTGAATGCCAGCTCTTTGAGCACT | | |
| | GGAAGACCCTCATGAACCGACACGACCAAGAGCTGGAGAAGGACACTGTCACAGAGAGAAAAGCCAC | | |
| | AGAGCCACCCCAGGGCCTATCTCGTGCCATGTCCTCGAGGGATGCCCTTCTGCAGGATGAGGAAGAG | | |
| | GAGGAAGAGGAGGCAGCTGAGAGCGAGGAGGATGACAACCTGTCGTCCATGCTGCACCAGCGTGCTG | | |
| | AGATCCCATGGCGAGCCTGCGCCAAGTACCTGTCCTCCGCCGGCATCCTGCTCCTGTCGTTGCTGGT | | |
| | CTTCTCACAGCTGCTCAAGCACATGGTCCTGGTGGCCATCGACTACTGCCTGGCCAAGTGGACCGAC | | |
| | AGCGCCCTGACCCTGACCCCTGCAGCCAGGAACTGCTCCCTCAGCCAGGAGTGCACCCTCGACCAGA | | |
| | CTGTCTATGCCATGGTCTTCACGGTGCTCTGCAGCCTGGGCATTGTCCTGTCCCTCGTCACGTCTGT | | |
| | CACTGTGGAGTGGACAAGGCTGAAGGTGGCCAAGAGACTGCACCGCAGCCTGCTAAACCGGATCATC | | |
| | CTAGCCCCCATGAGGTTTTTTGAGACCACGCCCCTTGGGAGCATCCTGAACAGATTTTCATCTGACT | | |
| | GTAACACCATCGACCAGCACATCCCATCCACGCTGGAGTGCCTGAGCCGCTCCACCCTGCTCTGTGT | | |
| | CTCAGCCCTGGCCGTCATCTCCTATGTCACACCTGTGTTCCTCGTGGCCCTCTTGCCCCTGGCCATC | | |
| | GTGTGCTACTTCATCCAGAAGTACTTCCGGGTGGCGTCCAGGGACCTGCAGCAGCTGGATGACACCA | | |
| | CCCAGCTTCCACTTCTCTCACACTTTGCCGAAACCGTAGAAGGACTCACCACCATCCGGGCCTTCAG | | |
| | GTATGAGGCCCGGTTCCAGCAGAAGCTTCTCGAATACACAGACTCCAACAACATTGCTTCCCTCTTC | | |
| | CTCACAGCTGCCAACAGATGGCTGGAAGTCCGAATGGAGTACATCGCTGCATGTGTGGTGCTCATCG | | |
| | CAGCCGTGACCTCCATCTCCAACTCCCTGCACAGCGAGCTCTCTGCTGGCCTGGTGGGCCTGGGCCT | | |
| | TACCTACGCCCTAATGGTCTCCAACTACCTCAACTGGATGGTGAGCAACCTGGCAGACATGGAGCTC | | |
| | CAGCTGCGCGCTGTGAAGCGCATCCATGGGCTCCTGAAAACCGAGGCAGAGAGCTACGAGGGGCTCC | | |
| | TGGCACCATCGCTGATCCCAAAGAACTGGCCAGACCAAGGGAAGATCCAGATCCAGAACCTGAGCGT | | |
| | GCGCTACGACACCTCCCTGAAGCCGGTGCTGAAGCACGTCAATGCCCTCATCTCCCCTGGACAGAAG | | |
| | ATCGGGATCTGCGGCCGCACCGGCAGTGGGAAGTCCTCCTTCTCTCTTGCCTTCTTCCGCATGGTGG | | |
| | ACACGTTCGAAGGGCACATCATCATTGATGGCATTGACATCGCCAAACTGCCGCTGCACACCCTGCC | | |
| | CTCACGCCTCTCCATCATCCTGCAGGACCCCGTCCTCTTCAGCGGCACCATCCGATTTAACCTCGAC | | |
| | CCTGAGAGGAAGTGCTCAGATAGCACACTGTGGGAGGCCCTGGAAATCGCCCAGCTGAAGCTCGTGG | | |
| | TGAAGGCACTGCCAGGAGGCCTCGATGCCATCATCACAGAAGGCGGGGAGAATTTCAGCCAGCGACA | | |
| | GAGGCAGCTGTTCTGCCTGGCCCGGGCCTTCGTGAGGAAGACCAGCATCTTCATCATGGACGAGGCC | | |
| | ACGGCTTCCATTGACATGGCCACGGAAAACATCCTCCAAAAGGTGGTGATGACAGCCTTCGCAGACC | | |
| | GCACTGTGGTCACCATCGCGCATCGAGTGCACACCATCCTGAGTGCAGACCTGGTGATCGTCCTGAA | | |
| | GCGGGGTGCCATCCTTGAGTTCGATAAGCCAGAGAAGCTGCTCAGCCGGAAGGACAGCGTCTTCGCC | | |
| | TCCTTCGTCCGTGCAGACAAGTGACCTGCCAGAGCCCAAGTCCCATCCCACATTCGGACCCTGCCCA | | |
| | TA | | |
| | ORF Start: ATG at 36 | | ORF Stop: TGA at 4779 |
| | SEQ ID NO: 224 | 1581 aa | MW at 177005.9 kD |
| NOV51a, | MPLAFCGSENHSAAYRVDQGVLNNGCFVDALNVVPHVFLLFITFFILFIGWGSQSSKVHIHHSTWLH | | |
| CG57308-01 | | | |
| Protein Sequence | FPGHNLRWILTFMLLFVLVCETAECILSDGVTESHHLHLYMPAGMAFMAAVTSVVYYHNIETSNFPK | | |
| | LLIALLVYWTLAFITKTIKFVKFLDHAIGFSQLRFCLTGLLVILYGMLLLVEVNVIRVRRYIFFKTP | | |
| | REVKPPEDLQDLGVRFLQPFVNLLSKGTYWWMNAFIKTAHKKPIDLRAIGKLPIANRALTNYQRLCE | | |
| | AFDAQVRKDIOGTOGARAIWOALSHAFGRRLVLSSTFRILADLLGFAGPLCIFGIVDHLGKENDVFO | | |
| | PKTQFLGVYFVSSQEFLANAYVLAVLLFLALLLQRTFLQASYYVAIETGINLRGAIQTRIYNKIMHL | | |
| | STSNLSMGEMTACQICNLVAIDTNQLMWFFFLCPNLWAMPVQIIVGVILLYYILGVSALTGAAVTIL | | |
| | LAPVQYFVATKLSQAQRSTLEYSNERLKQTNEMLRGIKLLKLYAWENIFRTRVETTRRKEMTSLRAF | | |
| | AIYTSISIFMNTAIPIAAVLITFVGHVSFFKEADFSPSVAFASLSLFHILVTPLFLLSSVVRSTVKA | | |
| | LVSVQKLSEFLSSAEIREEQCAPHEPTPQGPASKYQAVPLRVVNRKRPAREDCRGLTGPLQSLVPSA | | |
| | DGDADNCCVQIMGGYFTWTPDGIPTLSNITIRIPRGQLTMIVGQVGCGKSSLLLAALGEMQKVSGAV | | |
| | FWSSLPDSEIGEDPSPERETATDLDIRKRGPVAYASQKPWLLNATVEENIIFESPFNKQRYKMVIEA | | |
| | CSLQPDTDILPHGDQTQIGERGINLSGGQRQRISVARALYQHANVVFLDDPFSALDIHLSDHLMQAG | | |
| | ILELLRDDKRTVVLVTHKLQYLPHADWIIAMKDGTTQREGTLKDFQRSECQLFEHWKTLNNRQDQEL | | |
| | EKETVTERKATEPPQGLSRAMSSRDGLLQDEEEEEEEAAESEEDDNLSSMLHQRAEIPWRACAKYLS | | |
| | SAGILLLSLLVFSQLLKHMVLVATDYWLAKWTDSALTLTPAARNCSLSQECTLDQTVYAMVFTVLCS | | |
| | LGIVLCLVTSVTVEWTGLKVAKRLHRSLLNRIILAPMRFFETTPLGSILNRFSSDCNTIDQEIPSTL | | |
| | ECLSRSTLLCVSALAVISYVTPVFLVALLPLAIVCYFIQKYFRVASRDLQQLDDTTQLPLLSHFAET | | |
| | VEGLTTIRAFRYEARFQQKLLEYTDSNNIASLFLTAANRWLEVRNEYIGACVVLIAAVTSISNSLHR | | |
| | ELSAGLVGLGLTYALMVSNYLNWMVRNLADMELQLCAVKRIHGLLKTEAESYEGLLAPSLIPKNWPD | | |
| | QCKIQIQNLSVRYDSSLKPVLKHVNALISPGQKIGICGRTGSGKSSFSLAFFRMVDTFEGHIIIDGI | | |
| | DIAKLPLHTLRSRLSIILQDPVLFSGTIRFNLDPERKCSDSTLWEALEIAQLKLVVKALPGGLDAII | | |
| | TEGGENFSQGQRQLFCLARAFVRKTSIFINDEATASIDMATENILQKVVMTAFADRTVVTIAHRVHT | | |
| | ILSADLVIVLKRGAILEFDKPEKLLSRKDSVFASFVRADK | | |
| | SEQ ID NO: 225 | 4745 bp | |
| NOV51b, | CGGGGCCCGGGGGGCGGGGGCCTGACGGCCGGGCCGGGCGGCGGAGCTGCAAGGGACAGAGGCGCGG | | |
| CG57308-02 | | | |
| DNA Sequence | CACGCGCGCGGAGCCAGCGGAGCCAGCTGAGCCCGAGCCCAGCCCGCGCCCGCGCCGCCATGCCCCT | | |
| | GGCCTTCTGCGGCAGCGAGAACCACTCGGCCGCCTACCGGGTGGACCAGGGGGTCCTCAACAACGGC | | |
| | TGCTTTGTGGACGCGCTCAACGTGGTGCCGCACGTCTTCCTACTCTTCATCACCTTCCCCATCCTCT | | |
| | TCATTGGATGGGGAAGTCAGAGCTCCAAGGTGCACATCCACCACAGCACATGGCTTCATTTCCCCGG | | |
| | GCACAACCTGCGGTGGATCCTGACCTTCATGCTGCTCTTCGTCCTGGTGTGTCAGATTGCAGAGGGC | | |
| | ATCCTGTCTGATGGGGTGACCGAATCCCACCATCTGCACCTGTACATGCCAGCCGGGATCGCGTTCA | | |
| | TGGCTGCTGTCACCTCCGTGGTCTACTATCACAACATCGAGACTTCCAACTTCCCCAAGCTGCTAAT | | |
| | TGCCCTGCTGGTGTATTGGACCCTGGCCTTCATCACCAAGACCATCAAGTTTGTCAAGCTCTTGGAC | | |
| | CACGCCATCGGCTTCTCGCAGCTACGCTTCTGCCTCACAGGGCTGCTGGTGATCCTCTATGGGATGC | | |
| | TGCTCCTCGTGGAGGTCAATGTCATCAGGGTGACGAGATACATCTTCTTCAAGACACCGAGGGAGGT | | |
| | GAAGCCTCCCGAGGACCTGCAAGACCTGGGGGTACGCTTCCTGCAGCCCTTCGTGAATCTGCCGTCC | | |
| | AAACGCACCTACTGGTGGATGAACGCCTTCATCAAGACTGCCCACAAGAAGCCCATCGACTTGCGAG | | |
| | CCATCGGGAACCTGCCCATCGTTATGAGGGCCCTCACCAACTACCAACGGCTCTGCGAGGCCTTTGA | | |
| | CGCCCAGGTGCGGAAGGACATTCAGGGCACTCAAGGTGCCCGGGCCATCTGGCAGGCACTCAGCCAT | | |
| | GCCTTCGGGACGCGCCTGGTCCTCAGCAGCACTTTCCGCATCTTGGCCGACCTGCTGGGCTTCGCCG | | |
| | GCCACTGTCCATCTTTGGCATCGTGGACCACCTTGGGAAGGAGAAACGACGTCTTCCAGCCCAAGAC | | |
| | ACAATTTCTCGGGGTTTACTTTGTCTCATCCCAAGAGTTCCTTGCCAATGCCTACGTCTTAGCTGTG | | |
| | CTTCTGTTCCTTGCCCTCCTACTGCAAAGGACATTTCTGCAAGCATCCTACTATGTGGCCATTGAAA | | |
| | CTGGAATTAACTTGAGAGGAGCAATACAGACCAAGATTTACAATAAAATTATGCACCTGTCCACCTC | | |
| | CAACCTGTCCATGGGAGAAATGACTGCTGGACAGATCTGTAATCTGGTTGCCATCGACACCAATCAG | | |
| | CTCATGTGGTTTTTCTTCTTGTGCCCAAACCTCTGGGCTATGCCAGTACAGATCATTGTGGGTGTGA | | |
| | TTCTCCTCTACTACATACTCGGAGTCAGTGCCTTAATTGGAGCAGCTGTCATCATTCTACTGGCTCC | | |
| | TGTCCAGTACTTCGTGGCCACCAAGCTGTCTCAGGCCCAGCGGAGCACACTGGAGTATTCCAATGAG | | |
| | CGGCTGAGCAGACCAACGAGATCCTCCGCGGCATCAAGCTCCTGAAGCTGTACGCCTGGGAAGAACA | | |
| | TCTTCCGCACGCGGGTGGAGACGACCCGCAGGAAGGAGATGACCAGCCTCAGGGCCTTTGCCATCTA | | |
| | TACCTCCATCTCCATTTTCATGAACACGGCCATCCCCATTGCAGCTGTCCTCATAACTTTCGTGGGC | | |
| | CATGTCAGCTTCTTCAAAGAGGCCGACTTCTCGCCCTCCGTGGCCTTTGCCTCCCTCTCCCTCTTCC | | |
| | ATATCTTGGTCACACCGCTGTTCCTGCTGTCCAGTGTGGTCCGATCTACCGTCAAAGCTCTAGTGAG | | |
| | CGTGCAAAAGCTAAGCGAGTTCCTGTCCAGTGCAGAGATCCGTGAGGAGCAGTGTGCCCCCCATGAG | | |
| | CCCACACCTCAGGGCCCAGCCAGCAAGTACCAGGCGGTGCCCCTCAGGGTTGTGAACCGCAAGCGTC | | |
| | CAGCCCGGGAGGATTGTCGGGGCCTCACCGGCCCACTGCAGACCCTGGTCCCCAGTGCAGATGGCGA | | |
| | TGCTGACAACTGCTGTGTCCAGATCATGGCAGGCTACTTCACGTGGACCCCAGATGGAATCCCCACA | | |
| | CTGTCCAACATCACCATTCGTATCCCCCGAGGCCAGCTGACTATGATCGTGGGGCAGGTGGGCTCCG | | |
| | GCAAGTCCTCGCTCCTTCTAGCCGCACTGGGGGAGATGCAGAAGGTCTCAGGGGCTGTCTTCTGGAG | | |
| | CAGCCTTCCTGACAGCGAGATAGGAGAGGACCCCAGCCCAGAGCGGGAGACAGCGACCGACTTGGAT | | |
| | ATCAGGAAGAGAGGCCCCGTGGCCTATGCTTCGCAGAAACCATGGCTGCTAAATGCCACTGTGGAGG | | |
| | AGAACATCATCTTTGAGAGTCCCTTCAACAAACAACGGTACAAGATGGTCATTGAAGCCTGCTCTCT | | |
| | GCAGCCAGACATCGACATCCTGCCCCATGGAGACCAGACCCAGATTGGGGAACGGGGCATCAACCTG | | |
| | TCTGGTGGTCAACGCCACCGAATCAGTGTGGCCCGAGCCCTCTACCAGCACGCCAACGTTGTCTTCT | | |
| | TGGATGACCCCTTCTCAGCTCTGGATATCCATCTGAGTGACCACTTAATGCACGCCGGCATCCTTGA | | |
| | GCTGCTCCGGGACGACAAGAGGACAGTGGTCTTAGTGACCCACAAGCTACAGTACCTGCCCCATGCA | | |
| | GACTGGATCATTGCCATGAAGGATGGCACCATCCAGAGGGAGGGTACCCTCAAGGACTTCCAGAGGT | | |
| | CTGAATGCCAGCTCTTTGAGCACTGGAGACCCTCATGAACCGACAGGACCAAAGAGCTGGAGAAGGA | | |
| | GACTGTCACAGAGAGAAAACCCACAGAGCCACCCCAGGGCCTATCTCGTGCCATGTCCTCGAGGGAT | | |
| | GGCCTTCTGCAGGATGAGGAAGAGGAGGAAGAGGAGGCAGCTGAGAGCGAGGAGGATGACAACCTGT | | |
| | CGTCCATGCTGCACCAGCGTGCTGAGATCCCATGGCCAGCCTGCGCCAAGTACCTGTCCTCCGCCGG | | |
| | CATCCTGCTCCTGTCGTTGCTGGTCTTCTCACAGCTGCTCAAGCACATGGTCCTGGTGGCCATCGAC | | |
| | TACTGGCTGGCCAAGTGGACCGACAGCGCCCTGACCCTGACCCCTGCAGCCAGGAACTGCTCCCTCA | | |
| | GCCAGGAGTGCACCCTCGACCAGACTGTCTATGCCATCGTGTTCACGCTGCTCTGCAGCCTGGGCAT | | |
| | TGTGCTGTGCCTCGTCACGTCTGTCACTGTGGAGTGGACAGGGCTGAACGTCGCCAAGAGACTGCAC | | |
| | CGCAGCCTGCTAAACCGGATCATCCTAGCCCCCATGAGGTTTTTTGAGACCACCCCCCTTGGGAGCA | | |
| | TCCTGAACAGATTTTCATCTGACTGTAACACCATCGACCAGCACATCCCATCCACGCTAAAGTGCCT | | |
| | GAGCCGCTCCACCCTGCTCTGTGTCTCAGCCCTGGCCGTCATCTCCTATGTCACACCTGTGTTCCTC | | |
| | GTGGCCCTCTTGCCCCTGCCCATCGTGTGCTACTTCATCCAGAAGTACTTCCCGGTGGCGTCCAGGG | | |
| | ACCTGCAGCAGCTGGATGACACCACCCAGCTTCCACTTCTCTCACACTTTGCCGAAACCGTAGAAGG | | |
| | ACTCACCACCATCCGGGCCTTCAGGTATGAGGCCCGGTTCCAGCAGAACCTTCTCGAATACACAGAC | | |
| | TCCAACAACATTGCTTCCCTCTTCCTCAACAGCTGCCAACAGATGCTGGAAGTCCGAATGGAGTACA | | |
| | TCGGTGCATGTGTGGTGCTCATCGCAGCGGTGACCTCCATCTCCAACTCCCTGCACAGGGAGCTCTC | | |
| | TGCTGGCCTGGTGGGCCTGGGCCTTACCTACGCCCTAATGGTCTCCAACTACCTCAACTGGATGGTG | | |
| | AGGAACCTGGCAGACATGGAGCTCCAGCTCGGGGCTGTGAAGCGCATCCATGGGCTCCTGAAAACCG | | |
| | AGGCAGAGAGCTACGAGGGGCTCCTGGCACCATCGCTGAATCCCAAGAACTGGCCAGACCAAGGGAA | | |
| | GATCCAGATCCAGAACCTGAGCGTGCGCTACGACAGCTCCCTGAAGCCGGTGCTGAAGCACGTCAAT | | |
| | GCCCTCATCTCCCCTGGACAGAAGATCGGGATCTGCGGCCGCACCGGCAGTGGGAAGTCCTCCTTCT | | |
| | CTCTTGCCTTCTTCCGCATGGTGGACACGTTCGAAGGGACATCATCACAGAAGGCGGGGGAGAATTT | | |
| | CAGCCAGGGACAGAGGCAGCTGTTCTGCCTGGCCCGGGCCTTCGTGACGAAGACCAGCATCTTCATC | | |
| | ATGGACGAGGCCACCGCTTCCATTGACATGGCCACGGAAAAAACATCCTCCAAGCTAATGATGACAG | | |
| | CCTTCGCAGACCGCACTGTGGTCACCATCGCGCATCGAGTGCACACCATCCTGAGTGCAGACCTGGT | | |
| | GATCGTCCTGAGCGGGGTGCCATCCTTGAGTTCGATAAGCCAGAGAAGCTGCTCAGCCGGAAAGGAC | | |
| | AGCGTCTTCGCCTCCTTCGTCCGTGCAGACAAGTGACCTGCCAGAGCCCAAGTGCCATCCCACATTC | | |
| | GGACCCTGCCCATACCCCTGCCTGCGTTTTCTAACTGTAAATCACTTGTAAATAA | | |
| | ORF Start: ATG at 127 | | ORF Stop: TGA at 4657 |
| | SEQ ID NO: 226 | 1510 aa | MW at 169179.9 kD |

| | |
|---|---|
| NOV51b, | MPLAFCGSENHSAAYRVDQGVLNNGCFVDALNVVPHVFLLFITFPILFIGWGSQSSKVHIHHSTWLH |
| CG57308-02 | |
| Protein Sequence | FPCHNLRWILTFMLLFVLVCEIAEGILSDGVTESHHLHLYMPAGMAFMAAVTSVVYYHNIETSNFPK |
| | LLIALLVYWTLAFITKTIKFVKLLDHTGFSQLRFCLTGLLVILYGMLLLVEVNVIRRVRRYIFFKTP |
| | REVKPPEDLQDLGVRFLQPFVNLPSKGTYWWMNAFIKTAHKKPIDLRAIGKLPIVMRALTNYQRLCE |
| | AFDAQVRKDIQGTQGARAIWQALSHAFGRRLVLSSTFRILADLLGFAGPLCIFGIVDHLGKENDVFQ |
| | PKTQFLGVYFVSSQEFLANAYVLAVLLFLALLIQRTFLQASYYVAIETGINLRGAIQTKIYNKIMHL |
| | STSNLSMGEMTAGQICNLVAIDTNQLMWFFFLCPNLWANPVQIIVGVILLYYILCVSALIGAAVIIL |
| | LAPVQYFVATKLSQAQRSTLEYSNERLKQTNEMLRGIKLLKLYAWENIFRTRVETTRRKEMTSLRAF |
| | AIYTSISIFMNTAIPIAAVLITFVGHVSFFKEADFSPSVAFASLSLFHILVTPLFLLSSVVRSTVKA |
| | LVSVQKLSEFLSSAEIREEQCAPHEPTPQGPASKYQAVPLRVVNRKRPAREDCRGLTGPLQSLVPSA |
| | DGDADNCCVQIMGGYFTWTPDGIPTLSNITIRIPRGQLTMIVGQVGCGKSSLLLAALCEMQKVSGAV |
| | FWSSLPDSEIGEDPSPERETATDLDIRKRGPVAYASQKPWLLNATVEENIIFESPFNKQRYKMVIEA |
| | CSLQPDIDILPHGDQTQIGERGTNLSGGQRQRISVARALYQHANVVFLDDPESALDIHLSDHLMQAG |
| | ILELLRDDKRTVVLVTHKLQYLPHADWIIAMKDGTIQREGTLKDFQRSECQLFEHWKTLMNRQDQEL |
| | EKETVTERKATEPPQGLSRAMSSRDGLLQDEEEEEEEAAESEEDDNLSSMLHQRAEIPWRACAKYLS |
| | SAGILLLSLLVFSQLLKHMVLVAIDYWLAKWTDSALTLTPAARNCSLSQECTLDQTVYAMVFTVLCS |
| | LGIVLCLVTSVTVEWTGLKVAKRLHRSLLNRIILAPMRFFETTPLGSILNRFSSDCNTIDQHIPSTL |
| | ECLSRSTLLCVSALAVISYVTPVFLVALLFLAIVCYEIQKYFRVASRDLQQLDDTTQLPLLSHFAET |
| | VEGLTTIRAFRYEARFQQKLLEYTDSNNIASLFLTAANRWLEVRMEYIGACVVLIAAVTSISNSLHR |
| | ELSAGLVGLGLTYALMVSNYLNWMVRNLADMELQLGAVKRIHGLLKTEAESYEGLLAPSLIPKNWPD |
| | QGKIQIQNLSVRYDSSLKPVLKHVNALISPGQKIGICGRTGSGKSSFSLAFFRMVDTFECHIITEGG |
| | ENFSQGQRQLFCLARAFVRKTSIFINDEATASIDMATENILQKVVMTAFADRTVVTIAHRVHTILSA |
| | DLVIVLKRGAILEFDKPEKLLSRKDSVFASFVRADK |

Sequence comparison of the above protein sequences yields the following sequence relationships shown in Table 51B.

TABLE 51B
Comparison of NOV51a against NOV51b.

| | NOV51a Residues/ | Identities/Similarities |
|---|---|---|
| Protein Sequence | Match Residues | for the Matched Region |
| NOV51b | 1 . . . 1406 | 1285/1406 (91%) |
| | 1 . . . 1406 | 1286/1406 (91%) |

Further analysis of the NOV51a protein yielded the following properties shown in Table 51C.

TABLE 51C
Protein Sequence Properties NOV51a

| | |
|---|---|
| PSort analysis: | 0.8000 probability located in plasma membrane; |
| | 0.4000 probability located in Golgi body; 0.3000 |
| | probability located in endoplasmic reticulum |
| | (membrane); 0.3000 probability located in microbody |
| | (peroxisome) |
| SignalP analysis: | Cleavage site between residues 56 and 57 |

A search of the NOV51a protein against the Geneseq database, a proprietary database that contains sequences published in patents and patent publication, yielded several homologous proteins shown in Table 51D.

TABLE 51D
Geneseq Results for NOV51a

| | | | Identities/ | |
|---|---|---|---|---|
| | | NOV51a | Similarities | |
| | Protein/Organism/ | Residues/ | for the | |
| Geneseq | Length | Match | Matched | Expect |
| Identifier | [Patent #, Date] | Residues | Region | Value |
| AAW57412 | Homo sapiens | 1 . . . 1581 | 1530/1582 | 0.0 |
| | sulphonylurea | | (96%) | |
| | receptor - Homo | 1 . . . 1580 | 1540/1582 | |
| | sapiens, 1580 aa. | | (96%) | |
| | [WO9814571-A1, | | | |
| | 09-APR-1998] | | | |
| AAR77087 | Rat sulphonylurea | 1 . . . 1581 | 1477/1582 | 0.0 |
| | receptor - Rattus | | (93%) | |
| | sp, 1582 aa. | 1 . . . 1582 | 1509/1582 | |
| | [WO9528411-A1, | | (95%) | |
| | 26-OCT-1995] | | | |
| AAR77088 | Hamster | 1 . . . 1581 | 1469/1582 | 0.0 |
| | sulphonylurea | | (92%) | |
| | receptor - Cricetus | 1 . . . 1582 | 1506/1582 | |
| | sp, 1582 aa. | | (94%) | |
| | [WO9528411-A1, | | | |
| | 26-OCT-1995] | | | |
| AAR77084 | Rat sulphonylurea | 1 . . . 1290 | 1195/1291 | 0.0 |
| | receptor - Rattus | | (92%) | |
| | sp, 1498 aa. | 1 . . . 1291 | 1223/1291 | |
| | [WO9528411-A1, | | (94%) | |
| | 26-OCT-1995] | | | |
| AAR77085 | Hamster | 1 . . . 1290 | 1186/1291 | 0.0 |
| | sulphonylurea | | (91%) | |
| | receptor - Cricetus | 1 . . . 1291 | 1220/1291 | |
| | sp, 1498 aa. | | (93%) | |
| | [WO9528411-A1, | | | |
| | 26-OCT-1995] | | | |

In a BLAST search of public sequence datbases, the NOV51a protein was found to have homology to the proteins shown in the BLASTP data in Table 51E.

TABLE 51E
Public BLASTP Results for NOV51a

| | | | Identities/ | |
|---|---|---|---|---|
| | | NOV51a | Similarities | |
| Protein | | Residues/ | for the | |
| Accession | Protein/Organism/ | Match | Matched | Expect |
| Number | Length | Residues | Portion | Value |
| Q09428 | Sulfonylurea | 2 . . . 1581 | 1579/1580 | 0.0 |
| | receptor 1 - Homo | | (99%) | |
| | sapiens (Human), | 1 . . . 1580 | 1579/1580 | |
| | 1580 aa. | | (99%) | |
| Q09429 | Sulfonylurea | 2 . . . 1581 | 1512/1582 | 0.0 |
| | receptor 1 - Rattus | | (95%) | |
| | norvegicus (Rat), | 1 . . . 1581 | 1536/1582 | |
| | 1581 aa. | | (96%) | |
| Q09427 | Sulfonylurea | 2 . . . 1581 | 1498/1582 | 0.0 |
| | receptor 1 - | | (94%) | |
| | Cricetus cricetus | 1 . . . 1581 | 1530/1582 | |
| | (Black-bellied | | (96%) | |
| | hamster), 1581 aa. | | | |
| A56248 | sulfonylurea | 1 . . . 1581 | 1469/1582 | 0.0 |
| | receptor - golden | | (92%) | |
| | hamster, 1582 aa. | 1 . . . 1582 | 1506/1582 | |
| | | | (94%) | |
| Q95J92 | Sulphonylurea | 1 . . . 1580 | 1076/1581 | 0.0 |
| | receptor 2B - | | (68%) | |
| | Oryctolagus | 1 . . . 1548 | 1277/1581 | |
| | cuniculus | | (80%) | |
| | (Rabbit), 1549 aa. | | | |

PFam analysis predicts that the NOV51a protein contains the domains shown in the Table 51F.

TABLE 51F
Domain Analysis of NOV51a

| | NOV51a Match | Identities/Similarities | Expect |
|---|---|---|---|
| Pfam Domain | Region | for the Matched Region | Value |
| ABC_membrane | 318 . . . 590 | 53/287 (18%) | 3.6e−46 |
| | | 212/287 (74%) | |
| ABC_tran | 706 . . . 905 | 55/214 (26%) | 1.3e−34 |
| | | 154/214 (72%) | |
| ABC_membrane | 1011 . . . 1298 | 58/292 (20%) | 2.7e−51 |
| | | 222/292 (76%) | |
| PRK | 1374 . . . 1391 | 6/19 (32%) | 0.21 |
| | | 15/19 (79%) | |
| ABC_tran | 1371 . . . 1554 | 54/199 (27%) | 5.7e−36 |
| | | 129/199 (65%) | |

### Example 52

The NOV52 clone was analyzed, and the nucleotide and encoded polypeptide sequences are shown in Table 52A.

TABLE 52A
NOV52 Sequence Analysis

| | SEQ ID NO: 227 | 1404 bp | |
|---|---|---|---|
| NOV52a, | ATGGAGTACATGAGCACTGGAAGTGACAATAAAGAAGAGATTGATTTATTAATTAAACATTTAAATG | ~ | |
| CG93659-01 | | | |
| DNA Sequence | TGTCTGATGTAATAGACATTATGGAAAATCTTTATGCAAGTGAAGAGCCAGCAGTTTATGAACCCAG | | |
| | TCTAATGACCATGTGTCAAGACAGTAATCAAAACGATGAGCGTTCTAAGTCTCTGCTGCTTAGTGGC | | |
| | CAAGAGGTACCATGGTTGTCATCAGTCAGATATGGAACTGTGGAGGATTTGCTTGCTTTTGCAAACC | | |
| | ATATATCCACACTGCAAAGCATTTTTATGGAACAACGACCACAGGAATCTGGAATTTTATTAAACAT | | |
| | GGTCATCACTCCCCAAAATGGACGTTACCAAATAGATTCCGATGTTCTCCTGATCCCCTGGAAGCTG | | |
| | ACTTACAGGATATTGGTTCTGATTTTAAATTCCTCGGGGCGCCTTTGGAAGGTATACTTGGCTCAAG | | |
| | ATATAAAGACGAAGAAAAGAATGGCGTGTAAACTGATCCCAGTAGAACAATTTAAGCCATCTGATGT | | |
| | GGAAATTCAGGCTTGCTTCCGGCACGAGAACATCGCACAGCTGTATGGCGCAGTCCTGTGGGGTGAA | | |
| | ACTGTCCATCTCTTTATGGAAGCAGGCGAGGGAAGGGTCTGTTCTGGAGAACTGGAGAGCTCTAAAC | | |
| | CAATGAGAGAATTTGAAATTATTTGGGTGACAAAGCATGTTCTCAAGGGACTTGATTTTCTACACTC | | |
| | AAACAAGTGATCCATCATGATATTAAACCTAGCAACATTGTTTTCAATGTCCACAAAAGCTGTTTTG | | |
| | GTGGATTTTGGCCTAAGTGTTCAATGAACCGAAGATGTCTATTTTCCTAAGGACCTCCGAGGAACAG | | |
| | AGATTTACATGAGCCCAGAGGTCATCCTGTGCAGGGGCCATTCAACCAAGCAGACAATCTACAGCCT | | |
| | GGGGGCCACGCTCATCCACATGCAGACGGGCACCCCACCCTGGGTGAAGCGCTACCCTCGCTCAGCC | | |
| | TATCCCTCCTACCTGTACATAATCCACAAGCAAGCACCTCCACTGGAAGACATTGCAGATGACTGCA | | |
| | GTCCAGGGATGAGAGAGCTGATAGAAGCTTCCCTGGAGAGAAACCCCAATCACCCCCCAAGAGCCGC | | |
| | AGACCTACTAAAACATGAGGCCCTGAACCCGCCCAGAGAGGATCAGCCACGCTGTACGAGTCTGGAC | | |
| | TCTGCCCTCTTGGAGCGCAAGAGGCTGCTGAGTAGGAAGCAGCTGGAACTTCCTGAGAACATTGCTG | | |
| | ATTCTTCGTGCACACGAAGCACCGAGGAATCTGAGATGCTCAAGAGGCAACGCTCTCTCTACATCGA | | |
| | CCTCGGCGCTCTGGCTGGCTACTTCAATCTTGTTCGGCGACCACCAACGCTTGAATATGGCTGA | | |
| | ORF Start: ATG at 1 | | ORF Stop: TGA at 1402 |
| | SEQ ID NO: 228 | 467 aa | MW at 52896.9 kD |
| NOV52a, | MEYMSTGSDNKEEIDLLIKHLNVSDVIDIMENLYASEEPAVYEPSLMTMCQDSNQNDERSKSLLLSG | | |
| CG93659-01 | | | |
| Protein Sequence | QEVPWLSSVRYGTVEDLLAFANHISNTKHFYGQRPQESGILLLNMVITPQNGRYQIDSDVLLIPWLK | | |
| | TYRNIGSDFIPRGAFGKVYLAQDIKTKKRMACKLIPVDQFKFSDVEIQACFRHENIAELYGAVLWGE | | |
| | TVHLFNEAGEGGSVLEKLESCGPMREFEIIWVTRHVLKGLDFLHSKKVIHHDIKPSNIVFNSTKAVL | | |
| | VDFGLSVQMTEDVYFPKDLRGTEIYMSPEVILCRGHSTKADIYSLGATLIHMQTGTPPWVKRYPRSA | | |
| | YPSYLYIIHKQAPPLEDIADDCSPGMRELIEASLERNPNHRPRAADLLKHEALNPPREDQPRCTSLD | | |
| | SALLERKRLLSRKELELPENIADSSCTGSTEESEMLKRQRSLYIDLGALAGYFNLVRGPPTLEYG | | |
| | SEQ ID NO: 229 | 1430 bp | |
| NOV52b, | CTGACACTGCACTGAGCACTTTATGAGCTTGAACTCTGTTAATCCTCACGACCACCTCATGAGACTC | | |
| CG93659-03 | | | |
| DNA Sequence | TCCAGAAGAGCAACAGTAATGGAGTACAATGAGCACTGGAAGTGACAATAAAGAAGAGATTGATTTA | | |
| | TTAATTAAACATTTAAATGTGTCTGATGTAATAGACATTAATGGAATCTTTATGCAAAGTGAAGAGC | | |
| | CAGCAGTTTATGAACCCAGTCTAATGACCATGTGTCAAGACAGTAATCAAAACGATGAGCGTTCTAA | | |
| | GTCTCTGCTGCTTAGTGGCCAAGAGGTACCATGGTTGTCATCAGTCAGATACGGAACTGTGGAGGAT | | |
| | TTGCTTGCTTTTGCAAACCATATATCCAACACTGCAAAGCATTTTTATGGACAACGACCACAGGAAT | | |
| | CTGGAATTTTATTAAACATGGTCATCACTCCCCAAAATAAACGTTACCAAATAGATTCCGATGTTCT | | |
| | CCTGATCCCCTGCAAGCTGACTTACAGGAATATTGGTTCTGATTTTATTTCTCGGGGCGCCTTTAAA | | |
| | AAGGTATACTTGGCACAAGATATAAAGACGAAGGAAAAGAATGGCGTGTAACTGATCCCAGTAGATC | | |
| | ATTTAAGCCATCTGATGTGGAAATCCAAGGCTTGCTTCCGGCACGAGAACATCGCAGAGCTGTATAA | | |
| | CGCAGTCCTGTCGGGTGAAACTGTCCATCTCTTTATGGAAGCAGGCGAGGGAGGGTCTGTTCTGGAG | | |
| | AAACTGGAGAGCTGTGGACCAATGAGAGAATTTGAAATTAATTTGGGTGACAAGCATGTTCTCAAGG | | |
| | GACTTGATTTTCTACACTCAAAGAAAGTGATCCATCATGATATAAACATTTACATGAGCCCAGAGGT | | |
| | CATCCTGTGCAGGGGCCATTCAACCAAAGCAGACATCTACAGCCTGGGGGCCACGCTCATCCACATG | | |
| | CAGACGGGCACCCCACCCTGGGTGAAGCGCTACCCTCGCTCAGCCTATCCCTCCTACCTGTACATAA | | |
| | TCCACAAGCAAGCACCTCCACTGGAAGACATTGCAGATGACTGCAGTCCAGGGATGAGAGAGCTGAT | | |
| | AGAAGCTTCCCTGGAGAGAAACCCCAATCACCGCCCAAGAGCCGCAGACCTACTAAAACATGAGGCC | | |
| | CTGAACCCGCCCAGAGAGGATCAGCCACGCTGTCACAGTCTGGACTCTGCCCTCTTGGAGCGCAAGA | | |
| | GGCTGCTGAGTAGGAAGGAGCTGGAACTTCCTGAGAACATTGCTGATTCTTCGTGCACAGGAACCAC | | |
| | CGAGGAATCTGAGATGCTCAAGAGGCAACGCTCTCTCTACATCGACCTCGCCGCTCTGGCTGGCTAC | | |
| | TTCAATCTTGTTCGGGGACCACCAACGCTTGAATATGGCTGAAGGATGCCATGTTTGCTCTAAATTA | | |
| | AGACAGCATTGATCTCCTGGAGG | | |
| | ORF Start: ATG at 87 | | ORF Stop: TGA at 1380 |
| | SEQ ID NO: 230 | 431 aa | MW at 48882.2 kD |
| NOV52b, | MEYMSTGSKNKEEIDLLIKHLNVSDVIDIMENLYASEEPAVYEPSLMTMCQDSNQNDERSKSLLLSG | | |
| CG93659-03 | | | |
| Protein Sequence | QEVPWLSSVRRYGTVEDLLAFAHISNTAKHFYGQRPQESGILLNMVITPQNGRYQIDSDVLLIPWKL | | |
| | TYRNIGSDFISRGAFGKVYLAQDIKTKKRMACKLIPVDQFKPSDVEIQACFRHENIAELYGAVLWGE | | |
| | TVHLFMEAGEGGSVLEKLESCGPMREFEIIWVTKHVLKGLDFLHSKKVIHHDINIYMSPEVILCRGH | | |
| | STKADIYSLGATLIHMQTGTPPWVKRYPRSAYPSYLYIIHKQAPPLEDIADDCSPGMRELIEASLER | | |
| | NPNHRPRAADLLKHEALNPPREDQPRCQSLDSALLERKRLLSRKELELPENIADSSCTCSTEESEML | | |
| | KRQRSLYIDLGALAGYFNLVRGPPTLEYG | | |
| | SEQ ID NO: 231 | 1538 bp | |
| NOV52c, | CTGACACTGCACTGAGCACTTTATGAGCTTCAACTCTGTTAATCCTCACGACCACCTCATGAGACTC | | |
| CG93659-02 | | | |
| DNA Sequence | TCCAGAAAGAGCAACAGTAATGGAGTACATGAGCACTGGAAGTGACAATAAAGAAGAGATTGATTTA | | |
| | TTAATTAAACATTTAAATGTGTCTGATGTAATAGACATTATGGAAAATCTTTATGCAAGTGAAGAGC | | |
| | CAGCAGTTTATGAACCCAGTCTAATGACCATGTGTCAAAGACAGTAATCAAACGATGAGCGTTCTAA | | |
| | GTCTCTGCTGCTTAGTGGCCAAGACGTACCATGGTTGTCATCAGTCAGATACGGAACTGTGGACGAT | | |
| | TTGCTTGCTTTTGCAAACCATATATCCAACACTGCAAAGCATTTTTATGGACAACGACCACAGGAAT | | |
| | CTGGAATTTTATTAAACATGGTCATCACTCCCCAAAATGGACGTTACCAAATAGATTCCGATGTTCT | | |
| | CCTGATCCCCTGGAAGCTGACTTACAGGAATATTGGTTCTGATTTTATTTCTCGGGGCGCCTTTGGA | | |
| | AAGGTATACTTGGCACAAGATATAAAGACGAAGAAAAGAATGGCGTGTAAACTGATCCCAGTAGATC | | |
| | ATTTAAGCCATCTGATGTGGAAATCCAGGCTTGCTTCCGGCACAAAGAACATCGCAGAGCTGTATAA | | |
| | CGCAGTCCTGTGGGGTCAAACTGTCCATCTCTTTATGGAAGCAGGCGAGGGAGGGTCTGTTCTGGAG | | |
| | AAACTGGAGAGCTGTGGACCAATGAGAGAATTTAAAAATTATTTGGGTGACAGCATGTTCTCAAAGG | | |
| | GACTTCATTTTCTACACTCAGAAAGTAAAATCCACCATGATATTAAACCTAGCAACATTGTTTTCAT | | |
| | GTCCACAAAAGCTGTTTTGGTGGATTTTGGCCTAAGTGTTCAAAGACCGAAAGATGTCTATTTTCCT | | |
| | AAGGACCTCCGAGGAACAGAGATTTACATGAGCCCACAGGTCATCCTGTGCAGTGGCCATTCAACCA | | |
| | AGCACACATCTACAGCCTGGGGGCCACGCTCATCCACATGCAGACGGGCAACCCCACCCTGAATGAA | | |
| | GCGCTACCCTCGCTCAGCCTATCCCTCCTACCTGTACATAATCCACAAGCAAGCACCTCCACTGGAA | | |
| | GACATTGCAGATGACTGCAGTCCAGGGATGAGAGAGCTGATAGAAGCTTCCCTGGAGAGAAACCCCA | | |
| | ATCACCGCCCAAGAGCCGCAGACCTACTAAAACATGAGGCCCTGAACCCGCCCAGAGAAAATCAGCC | | |
| | ACGCTGTCAGAGTCTGGACTCTGCCCTCTTGGAGCGCAAGAGGCTGCTGAGTAGGAAGGAGCTGGAA | | |
| | CTTCCTGAGAACATTGCTGATTCTTCGTGCACAGGAAGCACCGAGGAATCTGAGATGCTCAACAGGC | | |
| | AACGCTCTCTCTACATCGACCTCGGCGCTCTGGCTGGCTACTTCAATCTTGTTCGGGGACCACCAAC | | |
| | GCTTGAATATGGCTGAAGGATGCCATGTTTGCTCTAAATTAAGACAGCATTGATCTCCTGGAGG | | |
| | ORF Start: ATG at 87 | | ORF Stop: TGA at 1488 |
| | SEQ ID NO: 232 | 467 aa | MW at 52844.7 kD |

| | |
|---|---|
| NOV52c, | MEYMSTGSDNKEEIDLLIKHLNVSDVIDIMENLYASEEPAVYEPSLMTMCQDSNQNDERSKSLLLSG |
| CG93659-02 | |
| Protein Sequence | QEVPWLSSVRYGTVEDLLAFANHISNTAKHFYGQRPQESGILLNMVITPQNGRYQIDSDVLLIPWKL |
| | TYRNIGSDFISRGAFGKVYLAQDIKTKKRMACKLIPVDQFKPSDVEIQACFRHENIAELYGAVLWGE |
| | TVHLFMEAGEGGSVLEKLESCGPMREFEIIWVTKHVLKGLDFLHSKXVIHHDIKPSNIVFMSTKAVL |
| | VDFGLSVQMTEDVYFPKDLRGTEIYMSPEVILCSGHSTKADIYSLGATLIHMQTGTPPWVKRYPRSA |
| | YPSYLYIIHKQAPPLEDIADDCSPGMRELIEASLERNPNHRPRAADLLKHEALNPPREDQPRCQSLD |
| | SALLERKRLLSRKELELPENIADSSCTGSTEESEMLKRQRSLYIDLGALAGYFNLVRGPPTLEYG |

Sequence comparison of the above protein sequences yields the following sequence relationships shown in Table 52B.

TABLE 52B
Comparison of NOV52a against NOV52b and NOV52c.

| | NOV52a Residues/ | Identities/Similarities |
|---|---|---|
| Protein Sequence | Match Residues | for the Matched Region |
| NOV52b | 1 . . . 467 | 413/467 (88%) |
| | 1 . . . 431 | 413/467 (88%) |
| NOV52c | 1 . . . 467 | 449/467 (96%) |
| | 1 . . . 467 | 449/467 (96%) |

Further analysis of the NOV52a protein yielded the following properties shown in Table 52C.

TABLE 52C
Protein Sequence Properties NOV52a

| | |
|---|---|
| PSort analysis: | 0.6500 probability located in cytoplasm; 0.1000 |
| | probability located in mitochondrial matrix space; |
| | 0.1000 probability located in lysosome (lumen); |
| | 0.0000 probability located in endoplasmic reticulum |
| | (membrane) |
| SignalP analysis: | No Known Signal Sequence Predicted |

A search of the NOV52a protein against the Geneseq database, a proprietary database that contains sequences published in patents and patent publication, yielded several homologous proteins shown in Table 52D.

TABLE 52D
Geneseq Results for NOV52a

| | | | Identities/ | |
|---|---|---|---|---|
| | | NOV52a | Similarities | |
| | Protein/Organism/ | Residues/ | for the | |
| Geneseq | Length | Match | Matched | Expect |
| Identifier | [Patent #, Date] | Residues | Region | Value |
| AAE05951 | Human cot | 1 . . . 467 | 467/467 | 0.0 |
| | oncoprotein encoded | | (100%) | |
| | by D14497 | 1 . . . 467 | 467/467 | |
| | oncogene - Homo | | (100%) | |
| | sapiens, 467 aa. | | | |
| | [US6265216-B1, | | | |
| | 24-JUL-2001] | | | |
| AAY79244 | Human COT - | 1 . . . 467 | 467/467 | 0.0 |
| | Homo sapiens, | | (100%) | |
| | 467 aa. | 1 . . . 467 | 467/467 | |
| | [WO200011191-A2, | | (100%) | |
| | 02-MAR-2000] | | | |
| AAE10313 | Human Tp12 | 1 . . . 467 | 466/467 | 0.0 |
| | protein - Homo | | (99%) | |
| | sapiens, 467 aa. | 1 . . . 467 | 466/467 | |
| | [WO200166559-A1, | | (99%) | |
| | 13-SEP-2001] | | | |
| AAE10314 | Rat Tp12 protein - | 1 . . . 467 | 439/467 | 0.0 |
| | Rattus sp, 467 aa. | | (94%) | |
| | [WO200166559-A1, | 1 . . . 467 | 454/467 | |
| | 13-SEP-2001] | | (97%) | |
| AAY79243 | Rat TPL-2 - Rattus | 1 . . . 467 | 438/467 | 0.0 |
| | norvegicus, 467 aa. | | (93%) | |
| | [WO200011191-A2, | 1 . . . 467 | 453/467 | |
| | 02-MAR-2000] | | (96%) | |

In a BLAST search of public sequence datbases, the NOV52a protein was found to have homology to the proteins shown in the BLASTP data in Table 52E.

TABLE 52E
Public BLASTP Results for NOV52a

| | | | Identities/ | |
|---|---|---|---|---|
| | | NOV52a | Similarities | |
| Protein | | Residues/ | for the | |
| Accession | Protein/Organism/ | Match | Matched | Expect |
| Number | Length | Residues | Portion | Value |
| P41279 | Mitogen-activated | 1 . . . 467 | 467/467 | 0.0 |
| | protein kinase kinase | | (100%) | |
| | kinase 8 (EC 2.7.1.-) | 1 . . . 467 | 467/467 | |
| | (COT proto-oncogene | | (100%) | |
| | serine/threonine-protein | | | |
| | kinase) (C-COT) | | | |
| | (Cancer Osaka thyroid | | | |
| | oncogene) - Homo | | | |
| | sapiens (Human), 467 aa. | | | |
| A48713 | serine/threonine-specific | 1 . . . 467 | 466/467 | 0.0 |
| | protein kinase cot, 58K | | (99%) | |
| | form - human, 467 aa. | 1 . . . 467 | 466/467 | |
| | | | (99%) | |
| Q63562 | Mitogen-activated | 1 . . . 467 | 438/467 | 0.0 |
| | protein kinase kinase | | (93%) | |
| | kinase 8 (EC 2.7.1.-) | 1 . . . 467 | 453/467 | |
| | (Tumor progression | | (96%) | |
| | locus 2) (TPL-2) - Rattus | | | |
| | norvegicus (Rat), 467 aa. | | | |
| Q07174 | Mitogen-activated | 1 . . . 467 | 435/467 | 0.0 |
| | protein kinase kinase | | (93%) | |
| | kinase 8 (EC 2.7.1.-) | 1 . . . 467 | 454/467 | |
| | (COT proto-oncogene | | (97%) | |
| | serine/threonine-protein | | | |
| | kinase) (C-COT) (Cancer | | | |
| | Osaka thyroid | | | |
| | oncogene) - Mus | | | |
| | musculus (Mouse), | | | |
| | 467 aa. | | | |
| A41253 | kinase-related | 1 . . . 397 | 379/397 | 0.0 |
| | transforming protein (EC | | (95%) | |
| | 2.7.1.-) - human, 415 aa. | 1 . . . 397 | 379/397 | |
| | | | (95%) | |

PFam analysis predicts that the NOV52a protein contains the domains shown in the Table 52F.

TABLE 52F
Domain Analysis of NOV52a

| | NOV52a Match | Identities/Similarities | Expect |
|---|---|---|---|
| Pfam Domain | Region | for the Matched Region | Value |
| pkinase | 146 . . . 388 | 74/279 (27%) | 4.7e−54 |
| | | 187/279 (67%) | |

### Example 53

The NOV53 clone was analyzed, and the nucleotide and encoded polypeptide sequences are shown in Table 53A.

TABLE 53A
NOV53 Sequence Analysis

| | SEQ ID NO: 233 | 1078 bp | |
|---|---|---|---|
| NOV53a, | GCGGCTACATTCGGCCCGGCCATGGCAGCGGCGCCCCTGAAAGTGTGCATCGTGGGCTCGGGGAACT | | |
| CG94521-01 | | | |
| DNA Sequence | GGGGTTCAGCTGTTGCAAAAATAATTGGTAATAACGTCAAGAAACTTCAGAAATTTGCCTCCACAGT | | |
| | CAAGATGTGCGTCTTTGAAGAAACAGTGAATGGCAGAAAACTGACAGACATCATAATAAATGACCAT | | |
| | GAAAATGTAAAATATCTTCCTGGACACAAGCTGCCAGAAAATGTGGTTCCCATGTCAAATCTTAGCG | | |
| | AGGCTGTGCAGGATGCAGACCTGCTGGTGTTTGTCATTCCCCACCAGTTCATTCACAGAATCTGTGA | | |
| | TGAGATCACTGGGAGAGTGCCCAAGAAGCGCTGGGAATCACCCTCATCAAAAGCATAAGACGAGGCC | | |
| | CCCGAGGGGCTGAAACTCATTTCTGACATCATCCCTGAGAAGATGGGTATTGACATCAGTGTGCTGA | | |
| | TGGGAGCCAACATTGCCAATGAGGTGGCTGCAGAGAAGTTCTGTGAGACCACCATCGGCAGCAAAGT | | |
| | AATGGAGAACGCCCTTCTCTTCAAAGAACTTCTGCAGACTCCAAATTTTCGAATTACGGTGGTTGAT | | |
| | GATGCAGACACTGTTGAACTCTGTGGTGCGCTTAAGAACATCGTAGCTGTGGGAGCTAAGTTCTGCG | | |
| | ACGGCCTCCGCTCTCGACACAACACCAAAGCGGCCGTCATCCGCCTGGGACTCATGGAAATGATTGC | | |
| | TTTTGCCAGGATCTTCTGCAAAGGCCAAGTGTCTACAGCCACCTTCCTAGAGAGCTGCAGGGTGGCC | | |
| | GACCTGATCACCACCTGTTACGGAGGGCGGAACCGCAGGGTGGCCGAGGCCTTCGCCAGAACTGGGA | | |
| | AGACCATTGAAGAGTTGGAGAAGGAGATGCTGAATGGGCAAAAGCTCCAAGGACCGCAGACTTCTGC | | |
| | TGAAGTGTACCGCATCCTCAAACAGAAGGGACTACTGGACAAGTTTCCATTGTTTACTGCAGTGTAT | | |
| | CAGATCTGCTACGAAAGCAGACCAGTTCAAGAGATGTTGTCTTGTCTTCAGAGCCATCCAGAGCATA | | |
| | CATAAA | | |
| | ORF Start: ATG at 22 | | ORF Stop: TAA at 1075 |
| | SEQ ID NO: 234 | 351 aa | MW at 38418.3 kD |
| NOV53a, | MAAAPLKVCIVGSGNWGSAVAKIIGNNVKKLQKFASTVKMWVFEETVNGRKLTDIINNDHENVKYLP | | |
| CG94521-01 | | | |
| Protein Sequence | GHKLPENVVAMSNLSEAVQDADLLVFVIPHQFIHRICDEITGRVPKKALGITLIKGIDEGPEGLKLI | | |
| | SDIIREKMGIDISVLMGANIANEVAAEKFCETTIGSKVMENGLLFKELLQTPNFRITVVDDADTVEL | | |
| | CGALKNIVAVGAGFCDGLRCGDNTKAAVIRLGLMEMIAFARIFCKGQVSTATFLESCGVADLITTCY | | |
| | GGRNRRVAEAFARTGKTIEELEKEMLNGQKLQGPQTSAEVYRILKQKGLLDKFPLFTAVYQICYESR | | |
| | PVQEMLSCLQSHPEHT | | |
| | SEQ ID NO: 235 | 936 bp | |
| NOV53b, | TACATTCGGCCCGGCCATGGCAGCGGCGCCCCTGAAAGTGTGCATCGTGGGCTCGGGGAACTGGGGT | | |
| CG94521-03 | | | |
| DNA Sequence | TCAGCTGTTGCAAAAATAATTGGTAATAATGTCAAGAAACTTCAGAAATTTGCCTCCACAGTCAAGA | | |
| | TGTGGGTCTTTGAAGAACAGTGAAATGGCAGAAAACTGACAGACATCATAAATAATGACCATGAAAA | | |
| | TGTAAAATATCTTCCTGGACACAAGCTGCCAGAAAATGTGCGCATAGACGAGGGCCCCGAGGAACTG | | |
| | AAGCTCATTTCTGACATCATCCGTGAGAAGATGGGTATTGACATCAGTGTGCTGATGGGAGCCAACA | | |
| | TTGCCAATGAGGTGGCTGCAGAGAAGTTCTGTGAGACCACCATCGGCAGCAAAGGAATGCAGAACGG | | |
| | CCTTCTCTTCAAGAACTTCTGCAGACTCCAAATTTTCGAATTACCCTGAATTGATGATGCAGACACT | | |
| | GTTGAACTCTGTGGTCCGCTTAAGAACATCGTAGCTGTGGGAGCTGGGTTCTGCGACGGCCTCCGCT | | |
| | GTGGAGACAACACCAAAGCGGCCGTCATCCGCCTGGGACTCATGGAAATGATTGCTTTTGCCAGGAT | | |
| | CTTCTGCAAAGGCCAAGTGTCTACAGCCACCTTCCTAGAGAGCTGCGGGGTGGCCGACCTGATCACC | | |
| | ACCTGTTACGGAGGGCCGGACCGCAGGGTGGCCGAGGCCTTCGCCAGAACTGGGAAGACCATTGAAG | | |
| | AGTTGGAGAAGGAGATCCTGAATGGGCAAAAGCTCCAAGGACCGCAGACTTCTGCTGAAGTGTACCG | | |
| | CATCCTCAAACAGAAGGGACTACTGGACAAGTTTCCATTGTTTACTGCACTGTATCAGATCTGCTAC | | |
| | GAAAGCAGACCAGTTCAAGAGATGTTGTCTTGTCTTCAGAGCCATCCAGAGCATACATAAAAAGG | | |
| | ORF Start: ATG at 17 | | ORF Stop: TAA at 929 |
| | SEQ ID NO: 236 | 304 aa | MW at 33235.2 kD |
| NOV53b, | MAAAPLKVCIVGSGNWGSAVAKIIGNNVKKLQKFASTVKMWVFEETVNGRKLTDIINNDHENVKYLP | | |
| CG94521-03 | | | |
| Protein Sequence | GHKLPENVGIDEGPEGLKLISDIIREKMGIDISVLMGANIANEVAAEKFCETTIGSKVMENGLLFKE | | |
| | LLQTPNFRITVVDDADTVELCGALKNIVAVGAGFCDGLRCGDNTKAAVIRLGLMEMIAFARIFCKGQ | | |
| | VSTATFLESCGVADLTTTCYGGRNRRVAEAEARTGKTIEELEKEMLNGQKLQGPQTSAEVYRILKQK | | |
| | GLLDKFPLFTAVYQICYESRPVQEMLSCLQSHPEHT | | |
| | SEQ ID NO: 237 | 1077 bp | |
| NOV53c, | TACATTCGGCCCGGCCATGGCACCGGCGCCCCTGAAACTGTGCATCGTGGGCTCCGCGAACTGGGGT | | |
| CG94521-02 | | | |
| DNA Sequence | TCAGCTGTTGCAAAAATAATTGCTAATAATGTCAAGAAACTTCAGAAATTTGCCTCCACAGTCAAGA | | |
| | TGTGGGTCTTTGAAGAAACAGTGAATGGCAGAAAACTGACAGACATCATAAATAATGACCATGAAAA | | |
| | TGTAAAATATCTTCCTGGACACAAGCTGCCAGAAAATGTGGTTGCCATGTCAAATCTTAGCGAGGCT | | |
| | GTGCAGGATGCAGACCTGCTGGTGTTTGTCATTCCCCACCAGTTCATTCACAGAATCTGTGATGAGA | | |
| | TCACTGGGAGAGTGCCCAAGAAAGCGCTCGGAATCACCCTCATCAAGGGCATAGACGAGGGCCCCGA | | |
| | GGGGCTGAAGCTCATTTCTGACATCATCCGTGAGAAGATGGGTATTGACATCAGTGTGCTGATGGGA | | |
| | GCCAACATTGCCAATGAGGTGGCTGCAGAGAAGTTCTGTGAGACCACCATCGGCAGCAAAGTAATGG | | |
| | AGAACGGCCTTCTCTTCAAAGAACTTCTGCAGACTCCAAATTTTCGAATTACCGTGGTTGATGATGC | | |
| | AGACACTGTTGAACTCTGTGGTGCGCTTAAGAACATCGTAGCTGTGGGAGCTGGGTTCTGCGACGGC | | |
| | CTCCGCTGTGGAGACAACACCAAAGCGGCCGTCATCCGCCTGGGACTCATCGAAATGATTGCTTTTG | | |
| | CCAGGATCTTCTGCAAAGGCCAAGTGTCTACAGCCACCTTCCTAGAGAGCTGCGGGGTGGCCGACCT | | |
| | GATCACCACCTGTTACCCAGGGCGGAACCGCAGGGTGGCCGAGGCCTTCGCCAGAACTGGGAAGACC | | |
| | ATTGAAGAGTTGGAGAAGGAGATGCTGAATGGGCAAAAGCTCCAAGGACCGCAGACTTCTGCTGAAG | | |
| | TGTACCGCATCCTCAAACAGAAGGGACTACTGGACAAGTTTCCATTGTTTACTGCAGTGTATCAGAT | | |
| | CTGCTACGAAAGCACACCAGTTCAAGAGATGTTGTCTTGTCTTCAGAGCCATCCAGAGCATACATAA | | |
| | AAAGG | | |
| | ORF Start: ATG at 17 | | ORF Stop: TAA at 1070 |
| | SEQ ID NO: 238 | 351 aa | MW at 38418.3 kD |

| | |
|---|---|
| NOV53c, | MAAAPLKVCIVGSGNWCSAVAKIIGNNVKKLQKFASTVKMWVFEETVNGRKLTDIINNDHENVKYLP |
| CG94521-02 | |
| Protein Sequence | GHKLPENVVAMSNLSEAVQDADLLVFVTPHQFIHRICDEITGRVPKKALGITLIKGIDEGPEGLKLI |
| | SDITREKMGIDISVLMGANIANEVAAEKFCETTIGSKVMENGLLFKELLQTPNFRITVVDDADTVEL |
| | CGALKNIVAVGAGFCDGLRCGDNTKAAVIRLGLMEMIAFARIFCKGQVSTATFLESCGVADLITTCY |
| | GGRNRRVAEAFARTGKTIEELEKEMLNGQKLQGPQTSAEVYRILKQKGLLDKFPLFTAVYQICYESR |
| | PVQEMLSCLQSHPEHT |

Sequence comparison of the above protein sequences yields the following sequence relationships shown in Table 53B.

TABLE 53B
Comparison of NOV53a against NOV53b and NOV53c.

| | | Identities/ |
|---|---|---|
| Protein | NOV53a Residues/ | Similarities for the |
| Sequence | Match Residues | Matched Region |
| NOV53b | 1 . . . 351 | 304/351 (86%) |
| | 1 . . . 304 | 304/351 (86%) |
| NOV53c | 1 . . . 351 | 351/351 (100%) |
| | 1 . . . 351 | 351/351 (100%) |

Further analysis of the NOV53a protein yielded the following properties shown in Table 53C.

TABLE 53C
Protein Sequence Properties NOV53a

| | |
|---|---|
| PSort | 0.6500 probability located in cytoplasm; |
| analysis: | 0.1000 probability located in |
| | mitochondrial matrix space; 0.1000 |
| | probability located in lysosome (lumen); |
| | 0.0000 probability located in endoplasmic |
| | reticulum (membrane) |
| SignalP | Cleavage site between residues 22 and 23 |
| analysis: | |

A search of the NOV53a protein against the Geneseq database, a proprietary database that contains sequences published in patents and patent publication, yielded several homologous proteins shown in Table 53D.

TABLE 53D
Geneseq Results for NOV53a

| | | NOV53a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABB64184 | Drosophila melanogaster | 3 . . . 350 | 212/349 (60%) | e−120 |
| | polypeptide SEQ ID NO | 2 . . . 349 | 263/349 (74%) | |
| | 19344 - Drosophila | | | |
| | melanogaster, 360 aa. | | | |
| | [WO200171042-A2, | | | |
| | 27 SEP. 2001] | | | |
| AAG08446 | Arabidopsis thaliana protein | 7 . . . 331 | 180/329 (54%) | 8e−95 |
| | fragment SEQ ID NO: 5988 - | 22 . . . 349 | 233/329 (70%) | |
| | Arabidopsis thaliana, 366 aa. | | | |
| | [EP1033405-A2, | | | |
| | 06 SEP. 2000] | | | |
| AAG08445 | Arabidopsis thaliana protein | 7 . . . 331 | 180/329 (54%) | 8e−95 |
| | fragment SEQ ID NO: 5987 - | 56 . . . 383 | 233/329 (70%) | |
| | Arabidopsis thaliana, 400 aa. | | | |
| | [EP1033405-A2, | | | |
| | 06 SEP. 2000] | | | |
| AAG08444 | Arabidopsis thaliana protein | 7 . . . 331 | 180/329 (54%) | 8e−95 |
| | fragment SEQ ID NO: 5986 - | 77 . . . 404 | 233/329 (70%) | |
| | Arabidopsis thaliana, 421 aa. | | | |
| | [EP1033405-A2, | | | |
| | 06 SEP. 2000] | | | |
| AAG39422 | Arabidopsis thaliana protein | 7 . . . 331 | 180/329 (54%) | 1e−94 |
| | fragment SEQ ID NO: 48774 - | 22 . . . 349 | 232/329 (69%) | |
| | Arabidopsis thaliana, 366 | | | |
| | aa. [EP1033405-A2, | | | |
| | 06 SEP. 2000] | | | |

In a BLAST search of public sequence datbases, the NOV53a protein was found to have homology to the proteins shown in the BLASTP data in Table 53E.

TABLE 53E
Public BLASTP Results for NOV53a

| | | NOV53a | | |
|---|---|---|---|---|
| Protein | | Residues/ | Identities/ | |
| Accession | | Match | Similarities for the | Expect |
| Number | Protein/Organism/Length | Residues | Matched Portion | Value |
| AAH28726 | KIAA0089 protein - Homo | 1 . . . 351 | 351/351 (100%) | 0.0 |
| | sapiens (Human), 351 aa. | 1 . . . 351 | 351/351 (100%) | |
| Q14702 | KIAA0089 protein - Homo | 1 . . . 351 | 351/351 (100%) | 0.0 |
| | sapiens (Human), 411 aa | 61 . . . 411 | 351/351 (100%) | |
| | (fragment). | | | |
| O57656 | Glycerol-3-phosphate | 3 . . . 350 | 265/349 (75%) | e−155 |
| | dehydrogenase [NAD+], | 2 . . . 350 | 306/349 (86%) | |
| | cytoplasmic (EC 1.1.1.8) | | | |
| | (GPD-C) (GPDH-C) - Fugu | | | |
| | rubripes (Japanese | | | |
| | pufferfish) (Takifugu | | | |
| | rubripes), 351 aa. | | | |
| Q98SJ9 | Glycerol-3-phosphate | 7 . . . 350 | 258/345 (74%) | e−152 |
| | dehydrogenase (EC 1.1.1.8) - | 5 . . . 349 | 301/345 (86%) | |
| | Salmo salar (Atlantic | | | |
| | salmon), 350 aa. | | | |
| AAH32234 | Glycerol-3-phosphate | 4 . . . 350 | 249/347 (71%) | e−149 |
| | dehydrogenase 1 (soluble) - | 2 . . . 348 | 297/347 (84%) | |
| | Homo sapiens (Human), 349 | | | |
| | aa. | | | |

PFam analysis predicts that the NOV53a protein contains the domains shown in the Table 53F.

TABLE 53F
Domain Analysis of NOV53a

| | | Identities/ | |
|---|---|---|---|
| | | Similarities | |
| Pfam | NOV53a Match | for the | Expect |
| Domain | Region | Matched Region | Value |
| NAD_Gly3P_dh | 5 . . . 344 | 167/365 (46%) | 2.1e−184 |
| | | 307/365 (84%) | |

### Example 54

The NOV54 clone was analyzed, and the nucleotide and encoded polypeptide sequences are shown in Table 54A.

TABLE 54A
NOV54 Sequence Analysis

| | SEQ ID NO: 239 | 1552 bp | |
|---|---|---|---|
| NOV54a, | TTATTCCCCACTTTACCTGGCTAATTGAAGTGTAACAAAAGCTTCATCCAGGAACATTCGCGCGGGA | | |
| CG96613-01 | | | |
| DNA Sequence | AACCTGGCGTACTGGCTGTGGCTTCTCTAGCGGGACTCGGCATGAGGCTGGCGCGGCTGCTTCGCGG | | |
| | AGCCGCCTTGGCCGGCCCGGGCCCGGGGCTGCGCGCCGCCGGCTTCAGCCGCAGCTTCAGCTCGGAC | | |
| | TCGGGCTCCAGCCCGGCGTCCGACCGCGGCGTTCCGGGCCAAATGGACTTCTACGCGCGCTTCTCGC | | |
| | CGTCCCCGCTCTCCATGAAGCAGTTCCTGGACTTCGGATCAGTGAATGCTTGTGAAAAGACCTCATT | | |
| | TATGTTTCTGCGCCAAGAGTTGCCTCTCAGACTGCCAAATATAATGAAAGAAATAAGTCTCCTTCCA | | |
| | GATAATCTTCTCAGGACACCATCCGTTCAATTGGTACAAAGCTGGTATATCCAGACTCTTCAGGAGC | | |
| | TTCTTGATTTTAAGGACAAAAGTGCTCAGGATGCTAAAGCTATTTATGACTTTACAGATACTGTGAT | | |
| | ACGGATCAGAAACCGACACAATGATGTCATTCCCACAATGGCCCAGGGTGTGATTCAATACAAGGAG | | |
| | AGCTTTGGCGTGGATCCTGTCACCAGCCAGAATGTTCAGTACTTTTTGGATCGATTCTACATGAGTC | | |
| | GCATTTCAATTAGAATGTTACTCAATCAGCACTCTTTATTGTTTGGTGGAAAAGGCAAAGGAAGTCC | | |
| | ATCTCATCGAAAACACATTCGAAGCATAAATCCAAACTGCAATGTACTTGAAGTTATTAAAGATGGC | | |
| | TATGAAAATGCTAGGCGTCTGTCTGATTTGTATTATATTAACTCTCCCGAACTAGAACTTGAAGAAC | | |
| | TAAATGCAAAATCACCAGGACACCCAATACAAGTGGTTTATGTACCATCCCATCTCTATCACATGGT | | |
| | GTTTGAACTTTTCAAGAATGCAATGACAGCCACTATGGAACACCATGCCAACAGAGGTGTTTACCCC | | |
| | CCTATTCAAGTTCATGTCACGCTGGGTAATGAGGATTTGACTGTGAAGATGAGTGACCGAGGAGGTG | | |
| | GCGTTCCTTTGAGGAAAATTGACAGACTTTTCAACTACATGTATTCAACTGCACCAAGACCTCGTGT | | |
| | TGAGACCTCCCGCGCAGTGCCTCTGCCTGGTTTTGGTTATGGATTGCCCATATCACGTCTTTACGCA | | |
| | CAATACTTCCAAGCAGACCTGAAGCTGTATTCCCTAGAGGGTTACGGGACAGATGCAGTTATCTACA | | |
| | TTAAGGCTCTGTCAACAGACTCAATAGAAAGACTCCCAGTGTATAACAAAGCTGCCTGGAACCATTA | | |
| | CAACACCAACCACGACGCTGATGACTGGTGCGTCCCCAGCAGAGAACCCAAAGACATGACGACGTTC | | |
| | CGCAGTGCCTAGACACACTGGGGACATCGGAAAATCCAAATGTGGCTTTTGTATTAAATTTGGAAGG | | |
| | TATGGTGTTCAGAACTATATTATACCAAGTACTTTATTTATCGTTTTCACAAAACTATTTGAGTAGA | | |
| | ATAAATGCAAA | | |
| | ORF Start: ATG at 109 | | ORF Stop: TAG at 1417 |
| | SEQ ID NO: 240 | 436 aa | MW at 49243.6 kD |
| NOV54a, | MRLARLLRGAALAGPGPGLRAAGFSRSFSSDSGSSPASERGVPGQVDFYARFSPSPLSMKQFLDFGS | | |
| CG96613-01 | | | |
| Protein Sequence | VNACEKTSFMFLRQELPVRLANIMXEISLLPDNLLRTPSVQLVQSWYTQSLQELLDFKDKSAEDAKA | | |
| | IYDFTDTVIRIRNRHNDVIPTMAQGVIEYKESFGVDPVTSQNVQYFLDRFYMSRISIRMLLNQHSLL | | |
| | FGGKGKGSPSHRKHIGSINPNCNVLEVIKDGYENARRLCDLYYINSPELELEELNAKSPCQPIQVVY | | |
| | VPSHLYTHMVFELFKNMRATMEHHANRGVYPPIQVHVTLGNEDLTVKMSDRGGGVPLRKIDRLFNYM | | |
| | YSTAPRPRVETSRAVPLAGFGYGLPISRLYAQYFQGDLKLYSLEGYGTDAVIYIKALSTDSIERLPV | | |
| | YNKAAWXHYNTNHEADDWCVFSREPKDMTTFRSA | | |
| | SEQ ID NO: 241 | 1612 bp | |
| NOV54b, | TTATTCCCCACTTTACCTGGCTAATTGAAGTGTAACAAAAGCTTCATCCACGAACATTGGCGCGGGA | | |
| CG96613-03 | | | |
| DNA Sequence | AACCTGGCGTACTGGCTGTGGCTTCTCTAGCGGGACTCGGCATGAGGCTGGCGCGGCTGCTTCGCGG | | |
| | AGCCGCCTTGGCCCGCCCGGGCCCGGGGCTGCGCGCCGCCCGCTTCAGCCGCAGCTTCAGCTCGGAC | | |
| | TCGGGCTCCAGCCCGGCGTCCGAGCGCGGCCTTCCGGGCCAGGTGGACTTCTACGCGCGCTTCTCGC | | |
| | CGTCCCCGCTCTCCATGAACCAGTTCCTGGACTTCGGATCAGTGAATGCTTGTGAAAAGACCTCATT | | |
| | TATGTTTCTGCGGCAAGAGTTGCCTGTCAGACTGGCAAATATATGAAAGAAATAAAGTCTCCTTCCA | | |
| | GATAATCTTCTCAGGACACCATCCGTTCAATTGGTACAAAGCTGGTATATCCAGAGTCTTCAGGAGC | | |
| | TTCTTGATTTTAAGGACAAAGTGCTGAGGATGCTAAAGCTATTTATGAAAAGGCCTAGAAGAACATG | | |
| | GTTGCAGGTCTCTAGTTTATGCTGTATGGCCTGCAAGATGATCTTTACAGATACTGTGATACGGATC | | |
| | AGAAACCGACACAATGATGTCATTCCCACAATGGCCCAGGCTGTGATTGAATACAAGGAGAGCTTTG | | |
| | GGGTGGATCCTGTCACCAGCCAGAATGTTCAGTACTTTTTGGATCGATTCTACATGAGTCGCATTTC | | |
| | AATTAGAATGTTACTCAATCAGCACTCTTTATTGTTTGGTGGAAAAGGCAAAGGAAGTCCATCTCAT | | |
| | CGAAACACATTGGAAGCATAAATCCAAACTGCAATGTACTTGAAAGTTATTAAACATGGCTATGAAA | | |
| | ATGCTAGGCGTCTGTGTGATTTGTATTATATTAACTCTCCCGAACTAGAACTTGAAGAACTAAATGC | | |
| | AAAATCACCAGGACAGCCAATACAAGTGGTTTATGTACCATCCCATCTCTATCACATGGTGTTTGAA | | |
| | CTTTTCAAGAATGCAATGAGAGCCACTATGGAACACCATGCCAACAGAGGTGTTTACCCCCCTATTC | | |
| | AAGTTCATGTCACGCTGGATAATGAGGATTTGACTGTGAAGATGAGTGACCGAGGAGGTGGCGTTCC | | |
| | TTTGAGGAAAATTGACAGACTTTTCAACTACATGTATTCAACTGCACCAAGACCTCGTGTTGAGACC | | |
| | TCCCGCGCAGTGCCTCTGGCTGGTTTTGGTTATGGATTGCCCATATCACGTCTTTACGCACAATACT | | |
| | TCCAACGAGACCTGAAGCTGTATTCCCTAGAGGGTTACGGGACAGATGCACTTATCTACATTAACGC | | |
| | TCTGTCAACAGACTCAATAGAAAGACTCCCAGTGTATAACATAGCTGCCTGGAAGCATTACAACACC | | |
| | AACCACGAGGCTGATGACTGGTGCGTCCCCAGCAGAGAACCCAAAGACATCACGACGTTCCGCAGTG | | |
| | CCTAGACACACTGGGGACATCGGAAAATCCAAATGTGGCTTTTGTATTAAATTTGGAAGGTATGGTG | | |
| | TTCAGAACTATATTATACCAAGTACTTTATTTATCGTTTTCACAAAACTATTTGAGTAGAATAAATG | | |
| | GAAA | | |
| | ORF Start: ATG at 109 | | ORF Stop: TAG at 1477 |
| | SEQ ID NO: 242 | 456 aa | MW at 51622.6 kD |
| NOV54b, | MRLARLLRGAALAGPGPGLRAAGFSRSFSSDSGSSPASERGVPGQVDFYARFSPSPLSMKQFLDFGS | | |
| CG96613-03 | | | |
| Protein Sequence | VNACEKTSFMFLRQELPVRLANIMKEISLLPDNLLRTPSVQLVQSWYIQSLQELLDFKDKSAEDAKA | | |
| | IYERPRRTWLQVSSLCCMACKMIFTDTVIRIRNRHNDVIPTMAQGVIEYKESFGVDPVTSQNVQYFL | | |
| | DRFYMSRISIRMLLNQHSLLFGGKGKGSPSHRKHIGSINPNCNVLEVIKDGYENARRLCDLYYINSP | | |
| | ELELEELNAKSPGQPTQVVYVPSHLYHMVFELFKNANRATMEHHANRGVYPPIQVHVTLGNEDLTVK | | |
| | MSDRGGGVPLRKIDRLFNYMYSTAPRPRVETSRAVPLAGFGYGLPISRLYAQYFQGDLKLYSLEGYG | | |
| | TDAVIYIKALSTDSIERLPVYNKAAWKHYNTNHEADDWCVPSREPKDMTTFRSA | | |
| | SEQ ID NO: 243 | 967 bp | |
| NOV54c, | TTATTCCCCACTTTACCTGGCTAATTGAAGTGTAACAAAAGCTTCATCCAGGAACATTGGCGCGGGA | | |
| CG96613-02 | | | |
| DNA Sequence | AACCTGGCGTACTGGCTGTGGCTTCTCTAGCGGGACTCGGCATGAGGCTGGCGCGGCTGCTTCGCGG | | |
| | AGCCGCCTTGGCCGGCCCGGGCCCGGGCCTGCGCGCCGCCGGCTTCAGCCGCAGCTTCAGCTCGGAC | | |
| | TCGGGCTCCAGCCCGGCGTCCGAGCGCGGCGTTCCGGGCCAGGTGGACTTCTACGCGCGCTTCTCGC | | |
| | CGTCCCCGCTCTCCATGAAGCAGTTCCTGGACTTCGGATCAGTCAATGCTTGTCAAAAGACCTCATT | | |
| | TATGTTTCTGCGGCAAGAGTTGCCTCTCAGACTGGCAAATATAATGAAAGAAATAAGTCTCCTTCCA | | |
| | GATAATCTTCTCAGGACACCATCCGTTCAATTGGTACAAAGCTGGTATATCCAGAGTCTTCAGGAGC | | |
| | TTCTTGATTTTAAGGACAAAAGTGCTGAGGATGCTAAAGCTATTTATGAAAGGCCTAGAACAACATG | | |
| | GTTGCAGGTCTCTAGTTTATGCTGTATGGCCTGCAAGATGATCTTTACAGATACTGTGATACGGATC | | |
| | AGAAACCGACACAATGATGTCATTCCCACAATGGCCCAGGGTGTGATTGAATACAAGGACAGCTTTG | | |
| | GGGTGGATCCTGTCACCAGCCAGAATGTTCAGTACTTTATTTATCGTTTTCACAAAACTATTTGAGT | | |
| | AGAATAAATGGAAACTGAATTCCATTTGTGCCCGTTAAACCTCCTAAAGGATGAAATTGCACCTATT | | |
| | TTACACCTATATTTTCACAGTTAATTGAACATATTTTTAAACAACTGTAGTTTTGGGCAACTTTTCA | | |
| | CTTTGTGGTAGACTTCAGAAGPGTGGAAATCTTCGGGTTTCTATAGGAAACTAGTTTTTTTTTTTTT | | |
| | AAAAAAATCCTTTCTTTTTTGTGGGCTAG | | |
| | ORF Start: ATG at 109 | | ORF Stop: TGA at 733 |
| | SEQ ID NO: 244 | 208 aa | MW at 23483.8 kD |

| | |
|---|---|
| NOV54c, | MRLARLLRGAALAGPGPGLRAAGFSRSFSSDSGSSPASERGVPGQVDFYARFSPSPLSMKQFLDFGS |
| CG96613-02 | |
| Protein Sequence | VNACEKTSFMFLRQELPVRLANIMKEISLLPDNLLRTPSVQLVQSWYIQSLQELLDFKDKSAEDAKA |
| | IYERPRRTWLQVSSLCCMACKMIFTDTVIRTRNRHNDVIPTMAQGVIEYKESFGVDPVTSQNVQYFI |
| | YRFHKTI |

Sequence comparison of the above protein sequences yields the following sequence relationships shown in Table 54B.

TABLE 54B
Comparison of NOV54a against NOV54b and NOV54c.

| | | Identities/ |
|---|---|---|
| | | Similarities |
| Protein | NOV54a Residues/ | for the |
| Sequence | Match Residues | Matched Region |
| NOV54b | 42 . . . 436 | 394/415 (94%) |
| | 42 . . . 456 | 395/415 (94%) |
| NOV54c | 42 . . . 185 | 140/164 (85%) |
| | 42 . . . 205 | 143/164 (86%) |

Further analysis of the NOV54a protein yielded the following properties shown in Table 54C.

TABLE 54C
Protein Sequence Properties NOV54a

| | |
|---|---|
| PSort | 0.4251 probability located in mitochondrial |
| analysis: | matrix space; 0.3802 probability |
| | located in microbody (peroxisome); 0.1914 |
| | probability located in lysosome |
| | (lumen); 0.1017 probability located in |
| | mitochondrial inner membrane |
| SignalP | Cleavage site between residues 22 and 23 |
| analysis: | |

A search of the NOV54a protein against the Geneseq database, a proprietary database that contains sequences published in patents and patent publication, yielded several homologous proteins shown in Table 54D.

TABLE 54D
Geneseq Results for NOV54a

| | | NOV54a | Identities/ | |
|---|---|---|---|---|
| | | Residues/ | Similarities for | |
| Geneseq | Protein/Organism/Length | Match | the Matched | Expect |
| Identifier | [Patent #, Date] | Residues | Region | Value |
| ABG16621 | Novel human diagnostic | 42 . . . 435 | 269/395 (68%) | e−162 |
| | protein #16612 - Homo | 21 . . . 413 | 331/395 (83%) | |
| | sapiens, 415 aa. | | | |
| | [WO200175067-A2, | | | |
| | 11 OCT. 2001] | | | |
| ABB58044 | Drosophila melanogaster | 26 . . . 420 | 219/401 (54%) | e−121 |
| | polypeptide SEQ ID NO 924 - | 2 . . . 396 | 288/401 (71%) | |
| | Drosophila melanogaster, | | | |
| | 413 aa. [WO200171042-A2, | | | |
| | 27 SEP. 2001] | | | |
| AAE07838 | Maize pyruvate | 40 . . . 401 | 144/374 (38%) | 2e−60 |
| | dehydrogenase kinase | 8 . . . 364 | 211/374 (55%) | |
| | (PDK)-2 - Zea mays, 364 aa. | | | |
| | [US6265636-B1, | | | |
| | 24 JUL. 2001] | | | |
| AAW64724 | A. thaliana PDHK protein | 57 . . . 401 | 142/357 (39%) | 3e−58 |
| | from clone YA5 - | 29 . . . 366 | 209/357 (57%) | |
| | Arabidopsis thaliana, 366 aa. | | | |
| | [WO9835044-A1, | | | |
| | 13 AUG. 1998] | | | |
| AAE07837 | Maize pyruvate | 40 . . . 401 | 135/371 (36%) | 4e−56 |
| | dehydrogenase kinase | 8 . . . 347 | 205/371 (54%) | |
| | (PDK)-1 - Zea mays, 347 aa. | | | |
| | [US6265636-B1, | | | |
| | 24 JUL. 2001] | | | |

In a BLAST search of public sequence datbases, the NOV54a protein was found to have homology to the proteins shown in the BLASTP data in Table 54E.

TABLE 54E
Public BLASTP Results for NOV54a

| | | NOV54a | Identities/ | |
|---|---|---|---|---|
| Protein | | Residues/ | Similarities | |
| Accession | | Match | for the | |
| Number | Protein/Organism/Length | Residues | Matched Portion | Expect Value |
| Q15118 | [Pyruvate dehydrogenase | 1 . . . 436 | 436/436 (100%) | 0.0 |
| | [lipoamide]] kinase isozyme | 1 . . . 436 | 436/436 (100%) | |
| | 1, mitochondrial precursor | | | |
| | (EC 2.7.1.99) (Pyruvate | | | |
| | dehydrogenase kinase isoform | | | |
| | 1) - Homo sapiens (Human), | | | |
| | 436 aa. | | | |
| Q63065 | [Pyruvate dehydrogenase | 1 . . . 436 | 402/436 (92%) | 0.0 |
| | [lipoamide]] kinase isozyme | 1 . . . 434 | 412/436 (94%) | |
| | 1, mitochondrial precursor | | | |
| | (EC 2.7.1.99) (Pyruvate | | | |
| | dehydrogenase kinase isoform | | | |
| | 1) (PDK P48) - Rattus | | | |
| | norvegicus (Rat), 434 aa. | | | |
| Q8R2U8 | Similar to pyruvate | 1 . . . 436 | 401/436 (91%) | 0.0 |
| | dehydrogenase kinase, | 1 . . . 432 | 412/436 (93%) | |
| | isoenzyme 1 - Mus musculus | | | |
| | (Mouse), 432 aa. | | | |
| Q15119 | [Pyruvate dehydrogenase | 37 . . . 434 | 277/398 (69%) | e−168 |
| | [lipoamide]] kinase isozyme | 11 . . . 405 | 340/398 (84%) | |
| | 2, mitochondrial precursor | | | |
| | (EC 2.7.1.99) (Pyruvate | | | |
| | dehydrogenase kinase isoform | | | |
| | 2) - Homo sapiens (Human), | | | |
| | 407 aa. | | | |
| I70159 | [pyruvate dehydrogenase | 37 . . . 434 | 276/398 (69%) | e−168 |
| | (lipoamide)] kinase (EC | 11 . . . 405 | 340/398 (85%) | |
| | 2.7.1.99) 2 - human, 407 aa. | | | |

PFam analysis predicts that the NOV54a protein contains the domains shown in the Table 54F.

TABLE 54F
Domain Analysis of NOV54a

| | | Identities/ | |
|---|---|---|---|
| | | Similarities | |
| Pfam | NOV54a Match | for the | Expect |
| Domain | Region | Matched Region | Value |
| HATPase_c | 268 . . . 393 | 32/134 (24%) | 8.5e−20 |
| | | 84/134 (63%) | |

### Example 55

The NOV55 clone was analyzed, and the nucleotide and encoded polypeptide sequences are shown in Table 55A.

TABLE 55A
NOV55 Sequence Analysis

| | SEQ ID NO: 245 | 2885 bp | |
|---|---|---|---|
| NOV55a, | CGGCACGCCCGGGACGCTTTCTCTGGCTGGTAACCGCTACTCCCGGACACCAGACCACCGCCTTCCG | | |
| CG96736-01 | | | |
| DNA Sequence | TACACAGGGGCCCGCATCCCACCCTCCCGGACCTAAGAGCCTGGGTCCCCTGTTTCCGGAGTCCGCT | | |
| | TCCCGGCCCCCAGATTCTCGCATCCCAGCCCTCAGTGTCCAAGACCCAGGCAGCCCGGGTCCCCCCC | | |
| | TCCCGGATCCACGCGTCCGGGATCTGCGCCACCAGAACCTAGCCTCCTGCAGACCTCCGCCATCTGG | | |
| | GGGCACTCAACCTCCTGGAGCCAACGGCCCCACGTCCCACCCAGAGAAACTCTCGTATTCCCAGCTC | | |
| | CTAGGGCCAAGACCCGCGCGCTCCGAACTCCCAGCTTTCGGACATCTGAAACACACGGGGCAGAGCA | | |
| | GAGAAGCCTCAGCGCCCAGCCTGGGGAATTTAAACACTCCAGCTTCCAAGAGCCAAGGAACTTCAGT | | |
| | GCTGTGAACTCACAACTCTAACCAGCCCTCCAAAGTTCCAGTCTCCAGGTGCTGTTACTCAACTCAG | | |
| | TCCTAGGAACGTCGGGTCCTGCGAAGGAGCCCAGCGCTCCCAAGCCAGCTTCCAGGCGCTAAGAAAC | | |
| | CCCGGTGCTTCCCATCATGGTGGCCGATCCTCCTCGAGACTCCAAGGGGCTCGCAGCGGCGGAGCCA | | |
| | CCCCCAACGGGGGCCTGGCAGCTGGCCTCCATCGAGGACCAAGGCGCGGCAGCAGGCGGCTACTGCG | | |
| | GTTCCCGGGACCTGCTCCGCCGCTGCCTTCGAGCCAACCTGCTTGTGCTGCTGACAGTGGTCGCCGT | | |
| | GGTCGCCGGCGTGGCGCTGGGACTGGGGGTGTCGGGGGCCGCGCGTGCGCTGGCGTTGGGCCCGGGA | | |
| | GCGCTTGAGGCCTTCGTCTTCCCGCGCGAGCTGCTGCTGCGTCTGCTGCGGATGATCATCTTGCCGC | | |
| | TGGTGGTGTGCAGCTTGATCGGCGGCGCCGCCAGCCTCGACCCCGGCGCGCTCGGCCGTCTGGCCCC | | |
| | CTGGGCGCTGCTCTTTTTCCTGGTCACCACGCTGCTGGCGTCGGCGCTCGGAGTGGGCTTGGCGCTG | | |
| | GCTCTGCAGCCGGGCGCCGCCTCCGCCGCCATCAACGCCTCCGTGGGAGCCGCGGGCAGTGCCGAAA | | |
| | ATGCCCCCAGCAAGGAAGTGCTCGATTCGTTCCTGGATCTTGCGAGAAATATCTTCCCTTCCAACCT | | |
| | GGTGTCAGCAGCCTTTCGCTCATACTCTACCACCTATGAAGAGAGGAATATCACCGGAACCAGGGTG | | |
| | AAGGTGCCCGTGGGGCAGGAGGTGGAGGGGATGAACATCCTGGGCTTGGTAGTGTTTGCCATCGTCT | | |
| | TTGGTGTGGCGCTGCGGAAGCTGGGGCCTGAGGGGAGCTGCTTAATCCGCTTCTTCAACTCCTTCAA | | |
| | TGAGGCCACCATGGTTCTGGTCTCCTCGATCATGTGGTACCCCCCTGTGGGCATCATGTTCCTGGTG | | |
| | GCTGGCAAGATCGTGGAGATGGAGGATGTGGGTTTACTCTTTGCCCGCCTTGGCAAATACATTCTGT | | |
| | GCTGCCTGCTGCGTCACGCCATCCATGGGCTCCTGGTACTGCCCCTCATCTACTTCCTCTTCACCCG | | |
| | CAAAAACCCCTACCGCTTCCTGTGGGGCATCGTGACGCCCCTGGCCACTGCCTTTGGGACCTCTTCC | | |
| | AGTTCCGCCACGCTGCCGCTGATGATGAAGTGCGTGGAGGAGAATAATGGCGTGGCCAAGCACATCA | | |
| | GCCGTTTCATCCTGCCCATCGGCGCCACCGTCAACATGGACGGTGCCGCGCTCTTCCAGTGCGTGGC | | |
| | CGCAGTGTTCATTGCACAGCTCAGCCAGCAGTCCTTGGACTTCCTAAAGATCATCACCATCCTGGTC | | |
| | ACGGCCACAGCGTCCAGCGTGGGGGCAGCGGGCATCCCTGCTGGAGGTGTCCTCACTCTGGCCATCA | | |
| | TCCTCGAAGCAGTCAACCTCCCGGTCGACCATATCTCCTTGATCCTGGCTGTGGACTGGCTAGTCGA | | |
| | CCGGTCCTGTACCGTCCTCAATGTAGAAGGTGACGCTCTGGGGGCAGGACTCCTCCAAAATTATGTG | | |
| | GACCGTACGGAGTCGAGAAGCACAGAGCCTGAGTTGATACAAGTGAAGAGTGAGCTGCCCCTGGATC | | |
| | CGCTGCCAGTCCCCACTGAGGAAGGAAACCCCCTCCTCAAACACTATCGGGGGCCCGCAGGGGATGC | | |
| | CACGGTCGCCTCTGAGAAGGAATCAGTCATGTAAACCCCGGGAGGGACCTTCCCTGCCCTGCTGGGG | | |
| | GTGCTCTTTGGACACTGGATTATGAGGAATGGATAAATGGATGAGCTAGGGCTCTGGGGGTCTGCCT | | |
| | GCACACTCTGGGGAGCCAGGGGCCCCAGCACCCTCCACGACAGGAGATCTGGGATGCCTGGCTGCTG | | |
| | GAGTACATGTGTTCACAAGGGTTACTCCTCAAAACCCCCAGTTCTCACTCATGTCCCCAACTCAAGG | | |
| | CTAGAAAACAGCAACATGGACAAATAATGTTCTGCTGCGTCCCCACCGTGACCTGCCTGGCCTCCCC | | |
| | TGTCTCAGGGAGCAGGTCACAGGTCACCATGGGGAATTCTAGCCCCCACTGGGGGGATGTTACAACA | | |
| | CCATGCTGGTTATTTTGGCGGCTGTAGTTGTGGCGGGATGTGTGTGTGCACGTGTGTGTGTGTGTGT | | |
| | GTGTGTGTGTGTGTGTGTGTTCTGTGACCTCCTGTCCCCATCGTACGTCCCACCCTGTCCCCAGATC | | |
| | CCCTATTCCCTCCACAATAACAGAAACACTCCCAGGGACTCTGGGGAGAGGCTGACCACAAATACCT | | |
| | GCTGTCACTCCAGAGGACATTTTTTTTAGCAATAAAATTGAGTGTCAACTATTAAAAAAAAAAAAAA | | |
| | AAAA | | |
| | ORF Start: ATG at 620 | | ORF Stop: TAA at 2243 |
| | SEQ ID NO: 246 | 541 aa | MW at 56620.6 kD |
| NOV55a, | MVADPPRDSKGLAAAEPPPTGAWQLASIEDQGAAAGGYCGSRDLVRRCLRANLLVLLTVVAVVAGVA | | |
| CG96736-01 | | | |
| Protein Sequence | LGLGVSCACGALALGPGALEAFVFPGELLLRLLRNIILPLVVCSLIGGAASLDPGALGRLGAWALLF | | |
| | FLVTTLLASALGVGLALALQPGAASAAINASVCAAGSAENAPSKEVLDSFLDLARNIFPSNLVSAAF | | |
| | RSYSTTYEERNITGTRVXVPVGQEVEGMNILGLVVFAIVFGVALRKLGPEGELLIRFFNSFNEATMV | | |
| | LVSWTMWYAPVGIMFLVAGKIVEMEDVGLLFARLGKYILCCLLGHAIHGLLVLPLIYFLFTRKNPYR | | |
| | FLWGIVTPLATAFGTSSSSATLPLMMKCVEENNGVAKHISRFILPIGATVNMDGAALFQCVAAVFIA | | |
| | QLSQQSLDFVKIITILVTATASSVGAAGIPAGGVLTLAIILEAVNLPVDHISLILAVDWLVDRSCTV | | |
| | LNVEGDALGAGLLQNYVDRTESRSTEPELIQVKSELPLDPLPVPTEEGNPLLKHYRGPAGDATVASE | | |
| | SEQ ID NO: 247 | 2017 bp | |
| NOV55b, | CGTACAACTCCGCCCCATTGACGCAAATGGGCGGTAGGCGTGTACGGTGGGAGGTCTATATAAGCAG | | |
| CG96736-02 | | | |
| DNA Sequence | AGCTCTCTGGCTAACTAGAGAACCCACTGCTTACTGGCTTATCGAAATTAATACGACTCACTATAGG | | |
| | GAGACCCAAGCTGGCTAGCGTTTAAACTTAAGCTTGGTACCGAGCTCGCATCCACTAGTCCAGTGTG | | |
| | GTGGAATTCCACCATGGTCGCCGATCCTCCTCGAGACTCCAAGGGCCTCGCAGCGGCGGACCCCACC | | |
| | GCCAACGGGGGCCTGGCGCTGGCCTCCATCGAGGACCAAGGCGCGGCAGCAGCCGGCTACTGCGGTT | | |
| | CCCCGGACCAGGTGCGCCGCTGCCTTCGAGCCAACCTCCTTGTGCTGCTGACAGTGGTGGCCCTGGT | | |
| | GGCCGGCGTCGCGCTGGGACTGGGGGTGTCGGGGGCCGGGGGTGCGCTGGCGTTGGGCCCGGAGCGC | | |
| | TTGAGCGCCTTCGTCTTCCCGGGCGAGCTGCTGCTGCGTCTGCTGCGGATGATCATCTTGCCGCTGG | | |
| | TGGTGTGCAGCTTGATCGGCGGCGCCGCCAGCCTGGACCCCGGCGCGCTCGGCCGTCTGAACGCCTG | | |
| | GGCGCTGCTCTTTTTCCTGGTCACCACGCTGCTGGCGTCGGCGCTCGGAGTAAGCTTGGCGCTGGCT | | |
| | CTGCAGCCGGGCGCCGCCTCCGCCGCCATCAACGCCTCCGTGGGAGCCGCGGGCAGTGCCGAAAATG | | |
| | CCCCCAGCAAGGAGGTGCTCGATTCGTTCCTGGATCTTGCGAGAAATATCTTCCCTTCCAACCTAAT | | |
| | GTCAGCAGCCTTTCGCTCATACTCTACCACCTATGAAGAGAGGAATATCACCGGAACCAGGGTGAAG | | |
| | GTGCCCGTGGGGCAGGAGGTGGAGGGGATGAACATCCTGGGCTTGGTAGTGTTTGCCATCGTCTTTG | | |
| | GTGTGGCGCTGCCGAAGCTGGGGCCTGAAGGGGAGCTGCTTATCCGCTTCTTCAACTCCTTCAATGA | | |
| | GGCCACCATGGTTCTGGTCTCCTGGATCATGTGGTATGCCCCTGTGGGCATCATGTTCCTCGTGGCT | | |
| | GGCAAGATCGTGGAGATGGAGGATGTGGGTTTACTCTTTGCCCGCCTTGGCAAGTACATTCTGTGCT | | |
| | GCCTGCTGGGTCACGCCATCCATGGGCTCCTGGTACTGCCCCTCATCTACTTCCTCTTCACCCGCAA | | |
| | AAACCCCTACCGCTTCCTGTGGGGCATCGTGACGCCGCTGGCCACTGCCTTTGGGACCTCTTCCAGT | | |
| | TCCGCCACGCTGCCGCTGATGATGAAGTGCGTGGAGGAGAATAATGGCGTGGCCAAGCACATCAGCC | | |
| | GTTTCATCCTGCCCATCGGCGCCACCGTCAACATGGACGGTGCCGCGCTCTTCCAGTGCGTGGCCGC | | |
| | AGTGTTCATTGCACAGCTCAGCCAGCAGTCCTTGGACTTCGTAAAGATCATCACCATCCTGGTCACG | | |
| | GCCACAGCGTCCAGCGTGGGGGCAGCGGGCATCCCTGCTGGAGGTGTCCTCACTCTGGCCATCATCC | | |
| | TCCAAGCAGTCAACCTCCCGGTCGACCATATCTCCTTGATCCTGGCTGTGGACTGGCTAGTCGACCG | | |
| | GTCCTGTACCGTCCTCAATGTAGAAGGTGACGCTCTGGGGGCAGGACTCCTCCAAAATTACGTGGAC | | |
| | CGTACGGAGTCGAGAAGCACAGAGCCTGAGTTGATACAAGTGAAGAGTGAGCTGCCCCTGGATCCGC | | |
| | TGCCAGTCCCCACTGAGGAAGGAAACCCCCTCCPCAAACACTATCGGGGGCCCGCAGGGGATGCCAC | | |
| | GGTCGCCTCTGAGAAGGAATCAGTCATGTAAGCGGCCGCTCGAGTCTAGAGGGCCCGTTTAAACCCG | | |
| | CTGATCAGCCTCGACTGTGCCTTCTAGTTGCCAGCCATCTGTTGTTTGCCCCTCCCCCGTGCCTTCC | | |
| | TTGACCCTGGAAGGTGCCACTCCCACTGTCCTTTCCTAATAAAATGAGGAAATTGCATCGCATTGTC | | |
| | TGAGTAG | | |
| | ORF Start: at 134 | | ORF Stop: TAA at 1838 |
| | SEQ ID NO: 248 | 568 aa | MW at 59557.8 kD |

| | |
|---|---|
| NOV55b, | GDPSWLAFKLKLGTELGSTSPVWWNSTMVADPPRDSKCLAAAEPTANGGLALASIEDQGAAAGGYCG |
| CG96736-02 | |
| Protein Sequence | SRDQVRRCLRANLLVLLTVVAVVAGVALGLGVSGAGGALALGPERLSAFVFPGELLLRLLRMIILPL |
| | VVCSLIGGAASLDPGALGRLGAWALLFFLVTTLLASALGVGLALALQPGAASAAINASVGAAGSAEN |
| | APSKEVLDSFLDLARNIFPSNLVSAAFRSYSTTYEERNITGTRVKVPVGQEVEGNNILGLVVFAIVF |
| | GVALRKLGPEGELLIRFFNSFNEATMVLVSWIMWYAPVGIMFLVAGKIVEMEDVGLLFARLGKYILC |
| | CLLGHAIHGLLVLPLIYFLFTRKNPYRFLWGTVTPLATAFGTSSSSATLPLMMKCVEENNGVAKHIS |
| | RFILPIGATVNMDGAALFQCVAAVFIAQLSQQSLDFVKIITILVTATASSVGAAGIPAGGVLTLAII |
| | LEAVNLPVDHISLILAVDWLVDRSCTVLNVEGDALCAGLLQNYVDRTESRSTEPELIQVKSELPLDP |
| | LPVPTEEGNPLLKHYRGPAGDATVASEKESVM |

Sequence comparison of the above protein sequences yields the following sequence relationships shown in Table 55B.

TABLE 55B
Comparison of NOV55a against NOV55b.

| | | Identities/ |
|---|---|---|
| | | Similarities |
| Protein | NOV55a Residues/ | for the |
| Sequence | Match Residues | Matched Region |
| NOV55b | 1 . . . 541 | 423/541 (78%) |
| | 28 . . . 568 | 423/541 (78%) |

Further analysis of the NOV55a protein yielded the following properties shown in Table 55C.

TABLE 55C
Protein Sequence Properties NOV55a

| | |
|---|---|
| PSort | 0.6000 probability located in plasma |
| analysis: | membrane; 0.4000 probability located in |
| | Golgi body; 0.3000 probability located |
| | in endoplasmic reticulum (membrane); |
| | 0.3000 probability located in microbody |
| | (peroxisome) |
| SignalP | Cleavage site between residues 70 and 71 |
| analysis: | |

A search of the NOV55a protein against the Geneseq database, a proprietary database that contains sequences published in patents and patent publication, yielded several homologous proteins shown in Table 55D.

TABLE 55D
Geneseq Results for NOV55a

| | | NOV55a | Identities/ | |
|---|---|---|---|---|
| | | Residues/ | Similarities for | |
| Geneseq | Protein/Organism/Length | Match | the Matched | |
| Identifier | [Patent #, Date] | Residues | Region | Expect Value |
| ABG61858 | Prostate cancer-associated | 1 . . . 541 | 531/541 (98%) | 0.0 |
| | protein #59 - Mammalia, 541 | 1 . . . 541 | 531/541 (98%) | |
| | aa. [WO200230268-A2, | | | |
| | 18 APR. 2002] | | | |
| AAR95044 | Apoptosis participating | 1 . . . 513 | 499/513 (97%) | 0.0 |
| | protein - Homo sapiens, 514 | 1 . . . 513 | 499/513 (97%) | |
| | aa. [JP08089257-A, | | | |
| | 09 APR. 1996] | | | |
| AAY78144 | Human neutral amino acid | 32 . . . 541 | 314/521 (60%) | e−161 |
| | transporter ASCT1 - Homo | 21 . . . 532 | 378/521 (72%) | |
| | sapiens, 532 aa. | | | |
| | [US6020479-A, | | | |
| | 01 FEB. 2000] | | | |
| AAY99961 | Human amino acid | 32 . . . 541 | 314/521 (60%) | e−161 |
| | transporter ASCT1 protein - | 21 . . . 532 | 378/521 (72%) | |
| | Homo sapiens, 532 aa. | | | |
| | [US6074828-A, | | | |
| | 13 JUN. 2000] | | | |
| AAY97139 | ASCT1 human neutral amino | 32 . . . 541 | 314/521 (60%) | e−161 |
| | acid transporter protein - | 21 . . . 532 | 378/521 (72%) | |
| | Homo sapiens, 532 aa. | | | |
| | [US6100085-A, | | | |
| | 08 AUG. 2000] | | | |

In a BLAST search of public sequence datbases, the NOV55a protein was found to have homology to the proteins shown in the BLASTP data in Table 55E.

TABLE 55E
Public BLASTP Results for NOV55a

| | | NOV55a | Identities/ | |
|---|---|---|---|---|
| Protein | | Residues/ | Similarities for | |
| Accession | | Match | the Matched | |
| Number | Protein/Organism/Length | Residues | Portion | Expect Value |
| AAD09814 | Neutral amino acid | 1 . . . 541 | 532/541 (98%) | 0.0 |
| | transporter - Homo sapiens | 1 . . . 541 | 532/541 (98%) | |
| | (Human), 541 aa. | | | |
| Q15758 | Neutral amino acid | 1 . . . 541 | 531/541 (98%) | 0.0 |
| | transporter B(0) (ATB(0)) | 1 . . . 541 | 531/541 (98%) | |
| | (Sodium-dependent neutral | | | |
| | amino acid transporter type | | | |
| | 2) (RD114/simian type D | | | |
| | retrovirus receptor) (Baboon | | | |
| | M7 virus receptor) - Homo | | | |
| | sapiens (Human), 541 aa. | | | |
| O19105 | Neutral amino acid | 1 . . . 541 | 459/542 (84%) | 0.0 |
| | transporter B(0) (ATB(0)) | 1 . . . 541 | 485/542 (88%) | |
| | (Sodium-dependent neutral | | | |
| | amino acid transporter type | | | |
| | 2) - Oryctolagus cuniculus | | | |
| | (Rabbit), 541 aa. | | | |
| Q95JC7 | Neutral amino acid | 1 . . . 541 | 465/542 (85%) | 0.0 |
| | transporter B(0) (ATB(0)) | 1 . . . 539 | 486/542 (88%) | |
| | (Sodium-dependent neutral | | | |
| | amino acid transporter type | | | |
| | 2) - Bos taurus (Bovine), 539 | | | |
| | aa. | | | |
| AAM94351 | Na+-dependent amino acid | 1 . . . 541 | 445/553 (80%) | 0.0 |
| | transporter ASCT2 - Rattus | 1 . . . 551 | 471/553 (84%) | |
| | norvegicus (Rat), 551 aa. | | | |

PFam analysis predicts that the NOV55a protein contains the domains shown in the Table 55F.

TABLE 55F
Domain Analysis of NOV55a

| | | Identities/ | |
|---|---|---|---|
| | | Similarities | |
| Pfam | NOV55a Match | for the | Expect |
| Domain | Region | Matched Region | Value |
| SDF | 54 . . . 485 | 195/465 (42%) | 1.5e−178 |
| | | 373/465 (80%) | |

### Example B: Sequencing Methodology and Identification of NOVX Clones

1. GeneCalling™ Technology: This is a proprietary method of performing differential gene expression profiling between two or more samples developed at CuraGen and described by Shimkets, et al., “Gene expression analysis by transcript profiling coupled to a gene database query” Nature Biotechnology 17:198-803 (1999). cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then digested with up to as many as 120 pairs of restriction enzymes and pairs of linker-adaptors specific for each pair of restriction enzymes were ligated to the appropriate end. The restriction digestion generates a mixture of unique cDNA gene fragments. Limited PCR amplification is performed with primers homologous to the linker adapter sequence where one primer is biotinylated and the other is fluorescently labeled. The doubly labeled material is isolated and the fluorescently labeled single strand is resolved by capillary gel electrophoresis. A computer algorithm compares the electropherograms from an experimental and control group for each of the restriction digestions. This and additional sequence-derived information is used to predict the identity of each differentially expressed gene fragment using a variety of genetic databases. The identity of the gene fragment is confirmed by additional, gene-specific competitive PCR or by isolation and sequencing of the gene fragment.

2. SeqCalling™ Technology: cDNA was derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then sequenced using CuraGen's proprietary SeqCalling technology. Sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

3. PathCalling™ Technology: The NOVX nucleic acid sequences are derived by laboratory screening of cDNA library by the two-hybrid approach. cDNA fragments covering either the full length of the DNA sequence, or part of the sequence, or both, are sequenced. In silico prediction was based on sequences available in CuraGen Corporation's proprietary sequence databases or in the public human sequence databases, and provided either the full length DNA sequence, or some portion thereof.

The laboratory screening was performed using the methods summarized below:

cDNA libraries were derived from various human samples representing multiple tissue types, normal and diseased states, physiological states, and developmental states from different donors. Samples were obtained as whole tissue, primary cells or tissue cultured primary cells or cell lines. Cells and cell lines may have been treated with biological or chemical agents that regulate gene expression, for example, growth factors, chemokines or steroids. The cDNA thus derived was then directionally cloned into the appropriate two-hybrid vector (Gal4-activation domain (Gal4-AD) fusion). Such cDNA libraries as well as commercially available cDNA libraries from Clontech (Palo Alto, Calif.) were then transferred from E.coli into a CuraGen Corporation proprietary yeast strain (disclosed in U.S. Pat. Nos. 6,057,101 and 6,083,693, incorporated herein by reference in their entireties).

Gal4-binding domain (Gal4-BD) fusions of a CuraGen Corportion proprietary library of human sequences was used to screen multiple Gal4-AD fusion cDNA libraries resulting in the selection of yeast hybrid diploids in each of which the Gal4-AD fusion contains an individual cDNA. Each sample was amplified using the polymerase chain reaction (PCR) using non-specific primers at the cDNA insert boundaries. Such PCR product was sequenced; sequence traces were evaluated manually and edited for corrections if appropriate. cDNA sequences from all samples were assembled together, sometimes including public human sequences, using bioinformatic programs to produce a consensus sequence for each assembly. Each assembly is included in CuraGen Corporation's database. Sequences were included as components for assembly when the extent of identity with another component was at least 95% over 50 bp. Each assembly represents a gene or portion thereof and includes information on variants, such as splice forms single nucleotide polymorphisms (SNPs), insertions, deletions and other sequence variations.

Physical clone: the cDNA fragment derived by the screening procedure, covering the entire open reading frame is, as a recombinant DNA, cloned into pACT2 plasmid (Clontech) used to make the cDNA library. The recombinant plasmid is inserted into the host and selected by the yeast hybrid diploid generated during the screening procedure by the mating of both CuraGen Corporation proprietary yeast strains N106′ and YULH (U.S. Pat. Nos. 6,057,101 and 6,083,693).

4. RACE: Techniques based on the polymerase chain reaction such as rapid amplification of cDNA ends (RACE), were used to isolate or complete the predicted sequence of the cDNA of the invention. Usually multiple clones were sequenced from one or more human samples to derive the sequences for fragments. Various human tissue samples from different donors were used for the RACE reaction. The sequences derived from these procedures were included in the SeqCalling Assembly process described in preceding paragraphs.

5. Exon Linking: The NOVX target sequences identified in the present invention were subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The PCR product derived from exon linking was cloned into the pCR2.1 vector from Invitrogen. The resulting bacterial clone has an insert covering the entire open reading frame cloned into the pCR2.1 vector. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported herein.

6. Physical Clone: Exons were predicted by homology and the intron/exon boundaries were determined using standard genetic rules. Exons were further selected and refined by means of similarity determination using multiple BLAST (for example, tBlastN, BlastX, and BlastN) searches, and, in some instances, GeneScan and Grail. Expressed sequences from both public and proprietary databases were also added when available to further define and complete the gene sequence. The DNA sequence was then manually corrected for apparent inconsistencies thereby obtaining the sequences encoding the full-length protein.

The PCR product derived by exon linking, covering the entire open reading frame, was cloned into the pCR2.1 vector from Invitrogen to provide clones used for expression and screening purposes.

### Example C: Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR). RTQ PCR was performed on an Applied Biosystems ABI PRISM® 7700 or an ABI PRISM® 7900 HT Sequence Detection System. Various collections of samples are assembled on the plates, and referred to as Panel 1 (containing normal tissues and cancer cell lines), Panel 2 (containing samples derived from tissues from normal and cancer sources), Panel 3 (containing cancer cell lines), Panel 4 (containing cells and cell lines from normal tissues and cells related to inflammatory conditions), Panel 5D/5I (containing human tissues and cell lines with an emphasis on metabolic diseases), AI_comprehensive_pancl (containing normal tissue and samples from autoinflammatory diseases), Panel CNSD.01 (containing samples from normal and diseased brains) and CNS_neurodegeneration_panel (containing samples from normal and Alzheimer's diseased brains).

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

First, the RNA samples were normalized to reference nucleic acids such as constitutively expressed genes (for example, β-actin and GAPDH). Normalized RNA (5 ul) was converted to cDNA and analyzed by RTQ-PCR using One Step RT-PCR Master Mix Reagents (Applied Biosystems; Catalog No. 4309169) and gene-specific primers according to the manufacturer's instructions.

In other cases, non-normalized RNA samples were converted to single strand cDNA (sscDNA) using Superscript II (Invitrogen Corporation; Catalog No. 18064-147) and random hexamers according to the manufacturer's instructions. Reactions containing up to 10 μg of total RNA were performed in a volume of 20 μl and incubated for 60 minutes at 42° C. This reaction can be scaled up to 50 μg of total RNA in a final volume of 100 μl. sscDNA samples are then normalized to reference nucleic acids as described previously, using 1×TaqMan® Universal Master mix (Applied Biosystems; catalog No. 4324020), following the manufacturer's instructions.

Probes and primers were designed for each assay according to Applied Biosystems Primer Express Software package (version I for Apple Computer's Macintosh Power PC) or a similar algorithm using the target sequence as input. Default settings were used for reaction conditions and the following parameters were set before selecting primers: primer concentration=250 nM, primer melting temperature (Tm) range=58°-60° C., primer optimal Tm=59° C., maximum primer difference=2° C., probe does not have 5′G, probe Tm must be 10° C. greater than primer Tm, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe, respectively. Their final concentrations were: forward and reverse primers, 900 nM each, and probe, 200 nM.

PCR conditions: When working with RNA samples, normalized RNA from each tissue and each cell line was spotted in each well of either a 96 well or a 384-well PCR plate (Applied Biosystems). PCR cocktails included either a single gene specific probe and primers set, or two multiplexed probe and primers sets (a set specific for the target clone and another gene-specific set multiplexed with the target probe). PCR reactions were set up using TaqMan® One-Step RT-PCR Master Mix (Applied Biosystems, Catalog No. 4313803) following manufacturer's instructions. Reverse transcription was performed at 48° C. for 30 minutes followed by amplification/PCR cycles as follows: 95° C. 10 min, then 40 cycles of 95° C. for 15 seconds, 60° C. for 1 minute. Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100.

When working with sscDNA samples, normalized sscDNA was used as described previously for RNA samples. PCR reactions containing one or two sets of probe and primers were set up as described previously, using 1×TaqMan® Universal Master mix (Applied Biosystems; catalog No. 4324020), following the manufacturer's instructions. PCR amplification was performed as follows: 95° C. 10 min, then 40 cycles of 95° C. for 15 seconds, 60° C. for 1 minute. Results were analyzed and processed as described previously.

Panels 1, 1.1, 1.2, and 1.3D

The plates for Panels 1, 1.1, 1.2 and 1.3D include 2 control wells (genomic DNA control and chemistry control) and 94 wells containing cDNA from various samples. The samples in these panels are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in these panels are widely available through the American Type Culture Collection (ATCC), a repository for cultured cell lines, and were cultured using the conditions recommended by the ATCC. The normal tissues found on these panels are comprised of samples derived from all major organ systems from single adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose.

In the results for Panels 1, 1.1, 1.2 and 1.3D, the following abbreviations are used:

ca.=carcinoma,

*=established from metastasis,

met=metastasis,

s cell var=small cell variant,

non-s=non-sm=non-small,

squam=squamous,

pl. eff=p1 effusion=pleural effusion,

glio=glioma,

astro=astrocytoma, and

neuro=neuroblastoma.

General_screening_panel_v1.4, v1.5 and v1.6

The plates for Panels 1.4, 1.5, and 1.6 include 2 control wells (genomic DNA control and chemistry control) and 94 wells containing cDNA from various samples. The samples in Panels 1.4, 1.5, and 1.6 are broken into 2 classes: samples derived from cultured cell lines and samples derived from primary normal tissues. The cell lines are derived from cancers of the following types: lung cancer, breast cancer, melanoma, colon cancer, prostate cancer, CNS cancer, squamous cell carcinoma, ovarian cancer, liver cancer, renal cancer, gastric cancer and pancreatic cancer. Cell lines used in Panels 1.4, 1.5, and 1.6 are widely available through the American Type Culture Collection (ATCC), a repository for cultured cell lines, and were cultured using the conditions recommended by the ATCC. The normal tissues found on Panels 1.4, 1.5, and 1.6 are comprised of pools of samples derived from all major organ systems from 2 to 5 different adult individuals or fetuses. These samples are derived from the following organs: adult skeletal muscle, fetal skeletal muscle, adult heart, fetal heart, adult kidney, fetal kidney, adult liver, fetal liver, adult lung, fetal lung, various regions of the brain, the spleen, bone marrow, lymph node, pancreas, salivary gland, pituitary gland, adrenal gland, spinal cord, thymus, stomach, small intestine, colon, bladder, trachea, breast, ovary, uterus, placenta, prostate, testis and adipose. Abbreviations are as described for Panels 1, 1.1, 1.2, and 1.3D.

Panels 2D, 2.2, 2.3 and 2.4

The plates for Panels 2D, 2.2, 2.3 and 2.4 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI) or from Ardais or Clinomics). The tissues are derived from human malignancies and in cases where indicated many malignant tissues have “matched margins” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologist at NDRI/CHTN/Ardais/Clinomics). Unmatched RNA samples from tissues without malignancy (normal tissues) were also obtained from Ardais or Clinomics. This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, CA), Research Genetics, and Invitrogen.

HASS Panel v 1.0

The HASS panel v 1.0 plates are comprised of 93 cDNA samples and two controls. Specifically, 81 of these samples are derived from cultured human cancer cell lines that had been subjected to serum starvation, acidosis and anoxia for different time periods as well as controls for these treatments, 3 samples of human primary cells, 9 samples of malignant brain cancer (4 medulloblastomas and 5 glioblastomas) and 2 controls. The human cancer cell lines are obtained from ATCC (American Type Culture Collection) and fall into the following tissue groups: breast cancer, prostate cancer, bladder carcinomas, pancreatic cancers and CNS cancer cell lines. These cancer cells are all cultured under standard recommended conditions. The treatments used (serum starvation, acidosis and anoxia) have been previously published in the scientific literature. The primary human cells were obtained from Clonetics (Walkersville, Md.) and were grown in the media and conditions recommended by Clonetics. The malignant brain cancer samples are obtained as part of a collaboration (Henry Ford Cancer Center) and are evaluated by a pathologist prior to CuraGen receiving the samples. RNA was prepared from these samples using the standard procedures. The genomic and chemistry control wells have been described previously.

ARDAIS Panel v 1.0

The plates for ARDAIS panel v 1.0 generally include 2 control wells and 22 test samples composed of RNA isolated from human tissue procured by surgeons working in close cooperation with Ardais Corporation. The tissues are derived from human lung malignancies (lung adenocarcinoma or lung squamous cell carcinoma) and in cases where indicated many malignant samples have “matched margins” obtained from noncancerous lung tissue just adjacent to the tumor. These matched margins are taken from the tissue surrounding (i.e. immediately proximal) to the zone of surgery (designated “NAT”, for normal adjacent tissue) in the results below. The tumor tissue and the “matched margins” are evaluated by independent pathologists (the surgical pathologists and again by a pathologist at Ardais). Unmatched malignant and non-malignant RNA samples from lungs were also obtained from Ardais. Additional information from Ardais provides a gross histopathological assessment of tumor differentiation grade and stage. Moreover, most samples include the original surgical pathology report that provides information regarding the clinical state of the patient.

Panel 3D, 3.1 and 3.2

The plates of Panel 3D, 3.1, and 3.2 are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection), NCI or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D, 3.1, 3.2, 1, 1.1., 1.2, 1.3D, 1.4, 1.5, and 1.6 are of the most common cell lines used in the scientific literature.

Panels 4D, 4R, and 4.1D Panel 4 includes samples on a 96 well plate (2 control wells, 94 test samples) composed of RNA (Panel 4R) or cDNA (Panels 4D/4.1D) isolated from various human cell lines or tissues related to inflammatory conditions. Total RNA from control normal tissues such as colon and lung (Stratagene, La Jolla, Calif.) and thymus and kidney (Clontech) was employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothelial cells, microvascular lung endothelial cells, human pulmonary aortic endothelial cells, human umbilical vein endothelial cells were all purchased from Clonetics (Walkersville, MD) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

Mononuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 μg/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶cells/ml in DMEM 5% FCS (Hyclone), 100 μM non essential amino acids (Gibco), 1 mM sodium pyruvate (Gibco), mercaptoethanol (5.5×10⁻⁵M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵M (Gibco), and OnmM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. CD45RO beads were then used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24,48 and 72 hours.

To prepare the primary and secondary Th1/Th2 and Tr1 cells, six-well Falcon plates were coated overnight with 10 μg/ml anti-CD28 (Pharmingen) and 21 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (4 ng/ml). IL-12 (5 ng/ml) and anti-IL4 (1 μg/ml) were used to direct to Th1, while IL-4 (5 ng/ml) and anti-IFN gamma (1 μg/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 ng/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵M (Gibco), and 10 mM Hepes (Gibco). CCD1106 cells were activated for 6 and 14 hours with approximately 5 ng/ml TNF alpha and 1 ng/ml IL-1 beta, while NCI-H292 cells were activated for 6 and 14 hours with the following cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20° C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37° C. for 30 minutes to remove contaminating genomic DNA, extracted once with phenol chloroform and re-precipitated with 1/10 volume of 3M sodium acetate and 2 volumes of 100% ethanol. The RNA was spun down and placed in RNAse free water. RNA was stored at −80° C.

Al_comprehensive panel_v1.0

The plates for Alcomprehensive panel_v1.0 include two control wells and 89 test samples comprised of cDNA isolated from surgical and postmortem human tissues obtained from the Backus Hospital and Clinomics (Frederick, Md.). Total RNA was extracted from tissue samples from the Backus Hospital in the Facility at CuraGen. Total RNA from other tissues was obtained from Clinomics.

Joint tissues including synovial fluid, synovium, bone and cartilage were obtained from patients undergoing total knee or hip replacement surgery at the Backus Hospital. Tissue samples were immediately snap frozen in liquid nitrogen to ensure that isolated RNA was of optimal quality and not degraded. Additional samples of osteoarthritis and rheumatoid arthritis joint tissues were obtained from Clinomics. Normal control tissues were supplied by Clinomics and were obtained during autopsy of trauma victims.

Surgical specimens of psoriatic tissues and adjacent matched tissues were provided as total RNA by Clinomics. Two male and two female patients were selected between the ages of 25 and 47. None of the patients were taking prescription drugs at the time samples were isolated.

Surgical specimens of diseased colon from patients with ulcerative colitis and Crohns disease and adjacent matched tissues were obtained from Clinomics. Bowel tissue from three female and three male Crohn's patients between the ages of 41-69 were used. Two patients were not on prescription medication while the others were taking dexamethasone, phenobarbital, or tylenol. Ulcerative colitis tissue was from three male and four female patients. Four of the patients were taking lebvid and two were on phenobarbital.

Total RNA from post mortem lung tissue from trauma victims with no disease or with emphysema, asthma or COPD was purchased from Clinomics. Emphysema patients ranged in age from 40-70 and all were smokers, this age range was chosen to focus on patients with cigarette-linked emphysema and to avoid those patients with alpha-lanti-trypsin deficiencies. Asthma patients ranged in age from 36-75, and excluded smokers to prevent those patients that could also have COPD. COPD patients ranged in age from 35-80 and included both smokers and non-smokers. Most patients were taking corticosteroids, and bronchodilators.

In the labels employed to identify tissues in the AI_comprehensive panel_v 1.0 panel, the following abbreviations are used:

AI=Autoimmunity

Syn=Synovial

Normal=No apparent disease

Rep22/Rep20=individual patients

RA=Rheumatoid arthritis

Backus=From Backus Hospital

OA=Osteoarthritis

(SS)(BA) (MF)=Individual patients

Adj=Adjacent tissue

Match control=adjacent tissues

-M=Male

-F=Female

COPD=Chronic obstructive pulmonary disease

AI.05 chondrosarcoma

The AI.05 chondrosarcoma plates are comprised of SW1353 cells that had been subjected to serum starvation, and treatment with cytokines that are known to induce MMP (1, 3 and 13) synthesis (eg. IL1beta). These treatments include: IL-1β (10 ng/ml), IL-1β+TNF-α (50 ng/ml), IL-1β+Oncostatin (50 ng/ml) and PMA (100 ng/ml). The SW1353 cells were obtained from ATCC (American Type Culture Collection) and were all cultured under standard recommended conditions. The SW1353 cells were plated at 3×10⁵ cells/ml (in DMEM medium-10% FBS) in 6-well plate. The treatment was done in triplicate, for 6 and 18 h. The supernatants were collected for analysis of MMP 1, 3 and 13 production and for RNA extraction. RNA was prepared from these samples using the standard procedures.

Panels 5D and 5I

The plates for Panel 5D and 5I include two control wells and a variety of cDNAs isolated from human tissues and cell lines with an emphasis on metabolic diseases. Metabolic tissues were obtained from patients enrolled in the Gestational Diabetes study. Cells were obtained during different stages in the differentiation of adipocytes from human mesenchymal stem cells. Human pancreatic islets were alsoh obtained.

In the Gestational Diabetes study subjects are young (18-40 years), otherwise healthy women with and without gestational diabetes undergoing routine (elective) Caesarean section. After delivery of the infant, when the surgical incisions were being repaired/closed, the obstetrician removed a small sample (<1 cc) of the exposed metabolic tissues during the closure of each surgical level. The biopsy material was rinsed in sterile saline, blotted and fast frozen within 5 minutes from the time of removal. The tissue was then flash frozen in liquid nitrogen and stored, individually, in sterile screw-top tubes and kept on dry ice for shipment to or to be picked up by CuraGen. The metabolic tissues of interest include uterine wall (smooth muscle), visceral adipose, skeletal muscle (rectus) and subcutaneous adipose. Patient descriptions are as follows:

Patient 2: Diabetic Hispanic, overweight, not on insulin

Patient 7-9: Nondiabetic Caucasian and obese (BMI>30)

Patient 10: Diabetic Hispanic, overweight, on insulin

Patient 11: Nondiabetic African American and overweight

Patient 12: Diabetic Hispanic on insulin

Adiocyte differentiation was induced in donor progenitor cells obtained from Osirus (a division of Clonetics/BioWhittaker) in triplicate, except for Donor 3U which had only two replicates. Scientists at Clonetics isolated, grew and differentiated human mesenchymal stem cells (HuMSCs) for CuraGen based on the published protocol found in Mark F. Pittenger, et al., Multilineage Potential of Adult Human Mesenchymal Stem Cells Science Apr. 2 1999: 143-147. Clonetics provided Trizol lysates or frozen pellets suitable for mRNA isolation and ds cDNA production. A general description of each donor is as follows:

Donor 2 and 3 U: Mesenchymal Stem cells, Undifferentiated Adipose

Donor 2 and 3 μM: Adipose, AdiposeMidway Differentiated

Donor 2 and 3 AD: Adipose, Adipose Differentiated

Human cell lines were generally obtained from ATCC (American Type Culture Collection), NCI or the German tumor cell bank and fall into the following tissue groups: kidney proximal convoluted tubule, uterine smooth muscle cells, small intestine, liver HepG2 cancer cells, heart primary stromal cells, and adrenal cortical adenoma cells. These cells are all cultured under standard recommended conditions and RNA extracted using the standard procedures. All samples were processed at CuraGen to produce single stranded cDNA.

Panel 5I contains all samples previously described with the addition of pancreatic islets from a 58 year old female patient obtained from the Diabetes Research Institute at the University of Miami School of Medicine. Islet tissue was processed to total RNA at an outside source and delivered to CuraGen for addition to panel 5I.

In the labels employed to identify tissues in the 5D and 5I panels, the following abbreviations are used:

GO Adipose=Greater Omentum Adipose

SK=Skeletal Muscle

UT=Uterus

PL=Placenta

AD=Adipose Differentiated

AM=Adipose Midway Differentiated

U=Undifferentiated Stem Cells

Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

In the labels employed to identify tissues in the CNS panel, the following abbreviations are used:

PSP=Progressive supranuclear palsy

Sub Nigra=Substantia nigra

Glob Palladus=Globus palladus

Temp Pole=Temporal pole

Cing Gyr=Cingulate gyrus

BA 4=Brodman Area 4

Panel CNS_Neurodegeneration_V1.0

The plates for Panel CNS_Neurodegeneration_V1.0 include two control wells and 47 test samples comprised of cDNA isolated from postmortem human brain tissue obtained from the Harvard Brain Tissue Resource Center (McLean Hospital) and the Human Brain and Spinal Fluid Resource Center (VA Greater Los Angeles Healthcare System). Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains six brains from Alzheimer's disease (AD) patients, and eight brains from “Normal controls” who showed no evidence of dementia prior to death. The eight normal control brains are divided into two categories: Controls with no dementia and no Alzheimer's like pathology (Controls) and controls with no dementia but evidence of severe Alzheimer's like pathology, (specifically senile plaque load rated as level 3 on a scale of 0-3; 0=no evidence of plaques, 3=severe AD senile plaque load). Within each of these brains, the following regions are represented: hippocampus, temporal cortex (Brodman Area 21), parietal cortex (Brodman area 7), and occipital cortex (Brodman area 17). These regions were chosen to encompass all levels of neurodegeneration in AD. The hippocampus is a region of early and severe neuronal loss in AD; the temporal cortex is known to show neurodegeneration in AD after the hippocampus; the parietal cortex shows moderate neuronal death in the late stages of the disease; the occipital cortex is spared in AD and therefore acts as a “control” region within AD patients. Not all brain regions are represented in all cases.

In the labels employed to identify tissues in the CNS_Neurodegeneration_V1.0 panel, the following abbreviations are used:

AD=Alzheimer's disease brain; patient was demented and showed AD-like pathology upon autopsy

Control=Control brains; patient not demented, showing no neuropathology

Control (Path)=Control brains; pateint not demented but showing sever AD-like pathology

SupTemporal Ctx=Superior Temporal Cortex

Inf Temporal Ctx=Inferior Temporal Cortex

A. CG106764-01: RHO/RAC-INTERACTING CITRON KINASE.

Expression of gene CG106764-01 was assessed using the primer-probe set Ag2100, described in Table AA. Results of the RTQ-PCR runs are shown in Tables AB, AC, AD, AE, AF, AG, AH and AI.

TABLE AA
Probe Name AG2100

| | | | Start | SEQ ID |
|---|---|---|---|---|
| Primers | Sequence | Length | Position | No |
| Forward | 5′-agatccctggaacagaggatt-3′ | 21 | 2446 | 249 |
| Probe | TET-5′-tgtctgaagccaataaacttgcagca-3′-TAMRA | 26 | 2474 | 250 |
| Reverse | 5′-ccttcatgttcctttgggtaa-3′ | 121 | 2513 | 251 |

TABLE AB
AI.05 chondrosarcoma

| | Rel. |
|---|---|
| | Exp. (%) |
| | Ag2100, |
| | Run |
| Tissue Name | 306913849 |
| 138353_PMA (18 hrs) | 9.3 |
| 138352_IL-1beta + Oncostatin M (18 hrs) | 5.5 |
| 138351_IL-1beta + TNFa (18 hrs) | 12.5 |
| 138350_IL-1beta (18 hrs) | 12.5 |
| 138354_Untreated-complete medium (18 hrs) | 13.2 |
| 138347_PMA (6 hrs) | 34.9 |
| 138346_IL-1beta + Oncostatin M (6 hrs) | 64.2 |
| 138345_IL-1beta + TNFa (6 hrs) | 44.8 |
| 138344_IL-1beta (6 hrs) | 25.5 |
| 138349_Untreated-serum starved (6 hrs) | 100.0 |
| 138348_Untreated-complete medium (6 hrs) | 41.2 |

TABLE AC
AI_comprehensive panel_v1.0

| | Rel. | Rel. |
|---|---|---|
| | Exp. (%) | Exp. (%) |
| | Ag2100, | Ag2100, |
| | Run | Run |
| Tissue Name | 211059880 | 212328504 |
| 110967 COPD-F | 0.5 | 0.8 |
| 110980 COPD-F | 1.5 | 1.2 |
| 110968 COPD-M | 0.4 | 0.6 |
| 110977 COPD-M | 1.5 | 1.9 |
| 110989 Emphysema-F | 4.2 | 6.0 |
| 110992 Emphysema-F | 2.8 | 2.9 |
| 110993 Emphysema-F | 0.9 | 0.8 |
| 110994 Emphysema-F | 0.7 | 0.4 |
| 110995 Emphysema-F | 2.0 | 5.4 |
| 110996 Emphysema-F | 2.2 | 2.4 |
| 110997 Asthma-M | 1.9 | 3.1 |
| 111001 Asthma-F | 1.4 | 2.7 |
| 111002 Asthma-F | 1.0 | 1.0 |
| 111003 Atopic Asthma-F | 4.0 | 2.2 |
| 111004 Atopic Asthma-F | 16.6 | 17.0 |
| 111005 Atopic Asthma-F | 7.2 | 5.5 |
| 111006 Atopic Asthma-F | 0.9 | 0.7 |
| 111417 Allergy-M | 1.9 | 2.4 |
| 112347 Allergy-M | 0.0 | 0.1 |
| 112349 Normal Lung-F | 0.0 | 0.0 |
| 112357 Normal Lung-F | 6.1 | 6.0 |
| 112354 Normal Lung-M | 1.5 | 2.3 |
| 112374 Crohns-F | 2.9 | 5.2 |
| 112389 Match Control Crohns-F | 9.0 | 6.8 |
| 112375 Crohns-F | 2.5 | 3.8 |
| 112732 Match Control Crohns-F | 3.8 | 5.4 |
| 112725 Crohns-M | 0.1 | 0.7 |
| 112387 Match Control Crohns-M | 1.0 | 1.4 |
| 112378 Crohns-M | 0.0 | 0.0 |
| 112390 Match Control Crohns-M | 2.5 | 1.8 |
| 112726 Crohns-M | 3.8 | 5.9 |
| 112731 Match Control Crohns-M | 3.6 | 6.7 |
| 112380 Ulcer Col-F | 4.9 | 4.9 |
| 112734 Match Control Ulcer Col-F | 12.6 | 12.0 |
| 112384 Ulcer Col-F | 6.6 | 10.2 |
| 112737 Match Control Ulcer Col-F | 4.2 | 6.1 |
| 112386 Ulcer Col-F | 0.5 | 1.2 |
| 112738 Match Control Ulcer Col-F | 7.5 | 7.9 |
| 112381 Ulcer Col-M | 0.1 | 0.1 |
| 112735 Match Control Ulcer Col-M | 2.9 | 2.3 |
| 112382 Ulcer Col-M | 6.7 | 8.4 |
| 112394 Match Control Ulcer Col-M | 0.5 | 0.5 |
| 112383 Ulcer Col-M | 12.1 | 14.6 |
| 112736 Match Control Ulcer Col-M | 3.5 | 5.3 |
| 112423 Psoriasis-F | 1.4 | 1.1 |
| 112427 Match Control Psoriasis-F | 2.9 | 1.8 |
| 112418 Psoriasis-M | 0.8 | 0.8 |
| 112723 Match Control Psoriasis-M | 6.1 | 7.4 |
| 112419 Psoriasis-M | 1.0 | 1.3 |
| 112424 Match Control Psoriasis-M | 0.4 | 1.2 |
| 112420 Psoriasis-M | 1.8 | 2.4 |
| 112425 Match Control Psoriasis-M | 2.2 | 2.7 |
| 104689 (MF) OA Bone-Backus | 12.1 | 13.2 |
| 104690 (MF) Adj “Normal” | 5.4 | 4.2 |
| Bone-Backus | | |
| 104691 (MF) OA Synovium-Backus | 43.2 | 35.6 |
| 104692 (BA) OA Cartilage-Backus | 0.9 | 0.4 |
| 104694 (BA) OA Bone-Backus | 16.8 | 16.7 |
| 104695 (BA) Adj “Normal” | 6.5 | 6.1 |
| Bone-Backus | | |
| 104696 (BA) OA Synovium-Backus | 24.0 | 24.1 |
| 104700 (SS) OA Bone-Backus | 12.2 | 35.1 |
| 104701 (SS) Adj “Normal” | 7.9 | 9.5 |
| Bone-Backus | | |
| 104702 (SS) OA Synovium-Backus | 8.2 | 7.9 |
| 117093 OA Cartilage Rep7 | 2.0 | 2.3 |
| 112672 OA Bone5 | 1.9 | 0.8 |
| 112673 OA Synovium5 | 0.3 | 1.2 |
| 112674 OA Synovial Fluid cells5 | 0.5 | 0.4 |
| 117100 OA Cartilage Rep14 | 0.4 | 0.3 |
| 112756 OA Bone9 | 100.0 | 100.0 |
| 112757 OA Synovium9 | 0.5 | 0.2 |
| 112758 OA Synovial Fluid Cells9 | 0.8 | 1.5 |
| 117125 RA Cartilage Rep2 | 1.0 | 0.6 |
| 113492 Bone2 RA | 2.8 | 3.6 |
| 113493 Synovium2 RA | 1.7 | 0.7 |
| 113494 Syn Fluid Cells RA | 0.9 | 2.1 |
| 113499 Cartilage4 RA | 2.1 | 1.8 |
| 113500 Bone4 RA | 1.8 | 2.5 |
| 113501 Synovium4 RA | 2.1 | 2.3 |
| 113502 Syn Fluid Cells4 RA | 1.0 | 0.8 |
| 113495 Cartilage3 RA | 2.5 | 2.6 |
| 113496 Bone3 RA | 2.0 | 2.1 |
| 113497 Synovium3 RA | 1.4 | 1.4 |
| 113498 Syn Fluid Cells3 RA | 2.9 | 3.2 |
| 117106 Normal Cartilage Rep20 | 0.1 | 0.7 |
| 113663 Bone3 Normal | 0.3 | 0.1 |
| 113664 Synovium3 Normal | 0.0 | 0.0 |
| 113665 Syn Fluid Cells3 Normal | 0.1 | 0.2 |
| 117107 Normal Cartilage Rep22 | 0.9 | 0.3 |
| 113667 Bone4 Normal | 0.4 | 0.7 |
| 113668 Synovium4 Normal | 1.0 | 1.1 |
| 113669 Syn Fluid Cells4 Normal | 1.0 | 0.7 |

TABLE AD
CNS_neurodegeneration_v1.0

| | Rel. |
|---|---|
| | Exp. (%) |
| | Ag2100, |
| | Run |
| Tissue Name | 207929343 |
| AD 1 Hippo | 5.2 |
| AD 2 Hippo | 9.3 |
| AD 3 Hippo | 6.7 |
| AD 4 Hippo | 7.2 |
| AD 5 Hippo | 100.0 |
| AD 6 Hippo | 16.5 |
| Control 2 Hippo | 17.7 |
| Control 4 Hippo | 3.4 |
| Control (Path) 3 Hippo | 4.4 |
| AD 1 Temporal Ctx | 15.7 |
| AD 2 Temporal Ctx | 26.4 |
| AD 3 Temporal Ctx | 12.3 |
| AD 4 Temporal Ctx | 24.3 |
| AD 5 Inf Temporal Ctx | 65.5 |
| AD 5 Sup Temporal Ctx | 20.9 |
| AD 6 Inf Temporal Ctx | 44.1 |
| AD 6 Sup Temporal Ctx | 59.0 |
| Control 1 Temporal Ctx | 9.5 |
| Control 2 Temporal Ctx | 34.6 |
| Control 3 Temporal Ctx | 0.0 |
| Control 3 Temporal Ctx | 10.4 |
| Control (Path) 1 Temporal Ctx | 68.8 |
| Control (Path) 2 Temporal Ctx | 49.7 |
| Control (Path) 3 Temporal Ctx | 8.5 |
| Control (Path) 4 Temporal Ctx | 55.5 |
| AD 1 Occipital Ctx | 31.6 |
| AD 2 Occipital Ctx (Missing) | 0.0 |
| AD 3 Occipital Ctx | 8.4 |
| AD 4 Occipital Ctx | 28.7 |
| AD 5 Occipital Ctx | 52.5 |
| AD 6 Occipital Ctx | 22.8 |
| Control 1 Occipital Ctx | 3.9 |
| Control 2 Occipital Ctx | 64.6 |
| Control 3 Occipital Ctx | 40.6 |
| Control 4 Occipital Ctx | 6.4 |
| Control (Path) 1 Occipital Ctx | 77.9 |
| Control (Path) 2 Occipital Ctx | 28.5 |
| Control (Path) 3 Occipital Ctx | 1.5 |
| Control (Path) 4 Occipital Ctx | 40.9 |
| Control 1 Parietal Ctx | 7.8 |
| Control 2 Parietal Ctx | 34.4 |
| Control 3 Parietal Ctx | 15.8 |
| Control (Path) 1 Parietal Ctx | 68.8 |
| Control (Path) 2 Parietal Ctx | 32.3 |
| Control (Path) 3 Parietal Ctx | 4.9 |
| Control (Path) 4 Parietal Ctx | 58.6 |

TABLE AE
Panel 1.3D

| | Rel. |
|---|---|
| | Exp. (%) |
| | Ag2100, |
| | Run |
| Tissue Name | 152517508 |
| Liver adenocarcinoma | 11.7 |
| Pancreas | 0.0 |
| Pancreatic ca. CAPAN 2 | 3.2 |
| Adrenal gland | 1.4 |
| Thyroid | 0.1 |
| Salivary gland | 0.1 |
| Pituitary gland | 2.1 |
| Brain (fetal) | 2.1 |
| Brain (whole) | 24.7 |
| Brain (amygdala) | 11.2 |
| Brain (cerebellum) | 2.7 |
| Brain (hippocampus) | 36.3 |
| Brain (substantia nigra) | 1.5 |
| Brain (thalamus) | 30.4 |
| Cerebral Cortex | 100.0 |
| Spinal cord | 2.5 |
| glio/astro U87-MG | 6.4 |
| glio/astro U-118-MG | 33.7 |
| astrocytoma SW1783 | 5.9 |
| neuro*; met SK-N-AS | 14.5 |
| astrocytoma SF-539 | 7.4 |
| astrocytoma SNB-75 | 5.8 |
| glioma SNB-19 | 1.0 |
| glioma U251 | 2.4 |
| glioma SF-295 | 0.9 |
| Heart (fetal) | 0.4 |
| Heart | 0.1 |
| Skeletal muscle (fetal) | 3.4 |
| Skeletal muscle | 0.1 |
| Bone marrow | 5.4 |
| Thymus | 2.1 |
| Spleen | 0.6 |
| Lymph node | 0.4 |
| Colorectal | 1.8 |
| Stomach | 1.0 |
| Small intestine | 1.6 |
| Colon ca. SW480 | 13.1 |
| Colon ca.* SW620 (SW480 met) | 4.5 |
| Colon ca. HT29 | 4.1 |
| Colon ca. HCT-116 | 5.0 |
| Colon ca. CaCo-2 | 5.9 |
| Colon ca. tissue (ODO3866) | 2.8 |
| Colon ca. HCC-2998 | 3.7 |
| Gastric ca.* (liver met) NCI-N87 | 2.3 |
| Bladder | 0.9 |
| Trachea | 0.7 |
| Kidney | 0.7 |
| Kidney (fetal) | 1.8 |
| Renal ca. 786-0 | 7.1 |
| Renal ca. A498 | 3.7 |
| Renal ca. RXF 393 | 3.1 |
| Renal ca. ACHN | 4.4 |
| Renal ca. UO-31 | 6.3 |
| Renal ca. TK-10 | 3.2 |
| Liver | 0.0 |
| Liver (fetal) | 3.8 |
| Liver ca. (hepatoblast) HepG2 | 3.2 |
| Lung | 0.3 |
| Lung (fetal) | 0.9 |
| Lung ca. (small cell) LX-1 | 6.6 |
| Lung ca. (small cell) NCI-H69 | 8.5 |
| Lung ca. (s. cell var.) SHP-77 | 7.5 |
| Lung ca. (large cell) NCI-H460 | 0.0 |
| Lung ca. (non-sm. cell) A549 | 0.2 |
| Lung ca. (non-s. cell) NCI-H23 | 10.4 |
| Lung ca. (non-s. cell) HOP-62 | 1.4 |
| Lung ca. (non-s. cl) NCI-H522 | 5.3 |
| Lung ca. (squam.) SW 900 | 3.2 |
| Lung ca. (squam.) NCI-H596 | 7.2 |
| Mammary gland | 0.2 |
| Breast ca.* (pl. ef) MCF-7 | 5.6 |
| Breast ca.* (pl. ef) MDA-MB-231 | 14.5 |
| Breast ca.* (pl. ef) T47D | 2.4 |
| Breast ca. BT-549 | 6.8 |
| Breast ca. MDA-N | 14.0 |
| Ovary | 2.2 |
| Ovarian ca. OVCAR-3 | 2.5 |
| Ovarian ca. OVCAR-4 | 0.8 |
| Ovarian ca. OVCAR-5 | 2.7 |
| Ovarian ca. OVCAR-8 | 3.2 |
| Ovarian ca. IGROV-1 | 2.0 |
| Ovarian ca.* (ascites) SK-OV-3 | 7.4 |
| Uterus | 0.0 |
| Placenta | 0.2 |
| Prostate | 0.2 |
| Prostate ca.* (bone met) PC-3 | 2.0 |
| Testis | 4.0 |
| Melanoma Hs688(A).T | 0.7 |
| Melanoma* (met) Hs688(B).T | 0.3 |
| Melanoma UACC-62 | 0.5 |
| Melanoma M14 | 7.2 |
| Melanoma LOX IMVI | 2.8 |
| Melanoma* (met) SK-MEL-5 | 5.8 |
| Adipose | 0.2 |

TABLE AF
Panel 2.2

| | Rel. |
|---|---|
| | Exp. (%) |
| | Ag2100, |
| | Run |
| Tissue Name | 174166901 |
| Normal Colon | 6.3 |
| Colon cancer (OD06064) | 13.4 |
| Colon Margin (OD06064) | 9.0 |
| Colon cancer (OD06159) | 4.5 |
| Colon Margin (OD06159) | 5.9 |
| Colon cancer (OD06297-04) | 3.8 |
| Colon Margin (OD06297-05) | 9.9 |
| CC Gr.2 ascend colon (ODO3921) | 4.4 |
| CC Margin (ODO3921) | 2.8 |
| Colon cancer metastasis (OD06104) | 1.7 |
| Lung Margin (OD06104) | 3.1 |
| Colon mets to lung (OD04451-01) | 9.6 |
| Lung Margin (OD04451-02) | 3.2 |
| Normal Prostate | 1.2 |
| Prostate Cancer (OD04410) | 0.0 |
| Prostate Margin (OD04410) | 0.7 |
| Normal Ovary | 2.8 |
| Ovarian cancer (OD06283-03) | 11.7 |
| Ovarian Margin (OD06283-07) | 3.0 |
| Ovarian Cancer 064008 | 1.1 |
| Ovarian cancer (OD06145) | 0.9 |
| Ovarian Margin (OD06145) | 0.0 |
| Ovarian cancer (OD06455-03) | 15.8 |
| Ovarian Margin (OD06455-07) | 1.8 |
| Normal Lung | 1.2 |
| Invasive poor diff. lung adeno (ODO4945-01 | 8.4 |
| Lung Margin (ODO4945-03) | 1.2 |
| Lung Malignant Cancer (OD03126) | 5.0 |
| Lung Margin (OD03126) | 0.6 |
| Lung Cancer (OD05014A) | 10.2 |
| Lung Margin (OD05014B) | 9.0 |
| Lung cancer (OD06081) | 10.1 |
| Lung Margin (OD06081) | 4.0 |
| Lung Cancer (OD04237-01) | 4.1 |
| Lung Margin (OD04237-02) | 2.0 |
| Ocular Melanoma Metastasis | 0.9 |
| Ocular Melanoma Margin (Liver) | 0.4 |
| Melanoma Metastasis | 10.4 |
| Melanoma Margin (Lung) | 2.0 |
| Normal Kidney | 5.0 |
| Kidney Ca, Nuclear grade 2 (OD04338) | 15.4 |
| Kidney Margin (OD04338) | 5.0 |
| Kidney Ca Nuclear grade 1/2 (OD04339) | 100.0 |
| Kidney Margin (OD04339) | 9.3 |
| Kidney Ca, Clear cell type (OD04340) | 14.0 |
| Kidney Margin (OD04340) | 11.3 |
| Kidney Ca, Nuclear grade 3 (OD04348) | 9.0 |
| Kidney Margin (OD04348) | 30.4 |
| Kidney malignant cancer (OD06204B) | 3.6 |
| Kidney normal adjacent tissue (OD06204E) | 10.5 |
| Kidney Cancer (OD04450-01) | 2.4 |
| Kidney Margin (OD04450-03) | 13.3 |
| Kidney Cancer 8120613 | 6.7 |
| Kidney Margin 8120614 | 1.2 |
| Kidney Cancer 9010320 | 1.7 |
| Kidney Margin 9010321 | 4.5 |
| Kidney Cancer 8120607 | 0.5 |
| Kidney Margin 8120608 | 1.7 |
| Normal Uterus | 1.1 |
| Uterine Cancer 064011 | 1.5 |
| Normal Thyroid | 0.0 |
| Thyroid Cancer 064010 | 0.6 |
| Thyroid Cancer A302152 | 5.3 |
| Thyroid Margin A302153 | 0.0 |
| Normal Breast | 3.0 |
| Breast Cancer (OD04566) | 8.1 |
| Breast Cancer 1024 | 2.9 |
| Breast Cancer (OD04590-01) | 14.8 |
| Breast Cancer Mets (OD04590-03) | 3.2 |
| Breast Cancer Metastasis (OD04655-05) | 5.4 |
| Breast Cancer 064006 | 3.1 |
| Breast Cancer 9100266 | 2.6 |
| Breast Margin 9100265 | 2.3 |
| Breast Cancer A209073 | 1.8 |
| Breast Margin A2090734 | 2.5 |
| Breast cancer (OD06083) | 17.1 |
| Breast cancer node metastasis (OD06083) | 14.7 |
| Normal Liver | 0.4 |
| Liver Cancer 1026 | 0.0 |
| Liver Cancer 1025 | 1.8 |
| Liver Cancer 6004-T | 1.1 |
| Liver Tissue 6004-N | 2.5 |
| Liver Cancer 6005-T | 1.6 |
| Liver Tissue 6005-N | 0.0 |
| Liver Cancer 064003 | 0.7 |
| Normal Bladder | 2.9 |
| Bladder Cancer 1023 | 1.5 |
| Bladder Cancer A302173 | 17.8 |
| Normal Stomach | 10.4 |
| Gastric Cancer 9060397 | 1.1 |
| Stomach Margin 9060396 | 0.7 |
| Gastric Cancer 9060395 | 2.8 |
| Stomach Margin 9060394 | 2.8 |
| Gastric Cancer 064005 | 6.0 |

TABLE AG
Panel 3D

| | Rel. |
|---|---|
| | Exp (%) |
| | Ag2100, |
| | Run |
| Tissue Name | 164796104 |
| Daoy- Medulloblastoma | 7.3 |
| TE671- Medulloblastoma | 3.8 |
| D283 Med- Medulloblastoma | 15.7 |
| PFSK-1- Primitive Neuroectodermal | 11.2 |
| XF-498- CNS | 21.2 |
| SNB-78- Glioma | 11.3 |
| SF-268- Glioblastoma | 7.6 |
| T98G- Glioblastoma | 12.0 |
| SK-N-SH- Neuroblastoma (metastasis) | 5.6 |
| SF-295- Glioblastoma | 12.4 |
| Cerebellum | 16.2 |
| Cerebellum | 3.6 |
| NCI-H292- Mucoepidermoid lung carcinoma | 14.0 |
| DMS-114- Small cell lung | 10.4 |
| cancer | |
| DMS-79- Small cell lung cancer | 100.0 |
| NCI-H146- Small cell lung cancer | 14.3 |
| NCI-H526- Small cell lung cancer | 19.8 |
| NCI-N417- Small cell lung cancer | 5.8 |
| NCI-H82- Small cell lung cancer | 10.2 |
| NCI-H157- Squamous cell lung cancer (metastasis) | 13.8 |
| NCI-H1155- Large cell lung cancer | 36.1 |
| NCI-H1299- Large cell lung cancer | 22.7 |
| NCI-H727- Lung carcinoid | 14.4 |
| NCI-UMC-11- Lung carcinoid | 25.9 |
| LX-1- Small cell lung cancer | 11.0 |
| Colo-205- Colon cancer | 12.7 |
| KM12- Colon cancer | 17.2 |
| KM20L2- Colon cancer | 7.0 |
| NCI-H716- Colon cancer | 19.5 |
| SW-48- Colon adenocarcinoma | 10.6 |
| SW1116- Colon adenocarcinoma | 7.7 |
| LS 174T- Colon adenocarcinoma | 9.8 |
| SW-948- Colon adenocarcinoma | 1.4 |
| SW-480- Colon adenocarcinoma | 7.6 |
| NCI-SNU-5- Gastric carcinoma | 14.9 |
| KATO III- Gastric carcinoma | 18.8 |
| NCI-SNU-16- Gastric carcinoma | 12.6 |
| NCI-SNU-1- Gastric carcinoma | 12.3 |
| RF-1- Gastric adenocarcinoma | 5.3 |
| RF-48- Gastric adenocarcinoma | 7.6 |
| MKN-45- Gastric carcinoma | 11.7 |
| NCI-N87- Gastric carcinoma | 9.3 |
| OVCAR-5- Ovarian carcinoma | 3.0 |
| RL95-2- Uterine carcinoma | 4.5 |
| HelaS3- Cervical adenocarcinoma | 9.0 |
| Ca Ski- Cervical epidermoid carcinoma (metastasis) | 21.0 |
| ES-2- Ovarian clear cell carcinoma | 11.7 |
| Ramos- Stimulated with PMA/ionomycin 6 h | 10.8 |
| Ramos- Stimulated with PMA/ionomycin 14 h | 6.2 |
| MEG-01- Chronic myelogenous leukemia | 5.8 |
| (megokaryoblast) | |
| Raji- Burkitt's lymphoma | 6.7 |
| Daudi- Burkitt's lymphoma | 14.8 |
| U266- B-cell plasmacytoma | 5.1 |
| CA46- Burkitt's lymphoma | 5.0 |
| RL- non-Hodgkin's B-cell lymphoma | 3.8 |
| JM1- pre-B-cell lymphoma | 11.5 |
| Jurkat- T cell leukemia | 12.5 |
| TF-1- Erythroleukemia | 9.9 |
| HUT 78- T-cell lymphoma | 14.7 |
| U937- Histiocytic lymphoma | 8.1 |
| KU-812- Myelogenous leukemia | 17.7 |
| 769-P- Clear cell renal carcinoma | 6.3 |
| Caki-2- Clear cell renal carcinoma | 9.5 |
| SW 839- Clear cell renal carcinoma | 5.2 |
| G401- Wilms' tumor | 6.3 |
| Hs766T- Pancreatic carcinoma (LN metastasis) | 15.7 |
| CAPAN-1- Pancreatic adenocarcinoma (liver | 8.6 |
| metastasis) | |
| SU86.86- Pancreatic carcinoma (liver metastasis) | 14.1 |
| BxPC-3- Pancreatic adenocarcinoma | 9.4 |
| HPAC- Pancreatic adenocarcinoma | 14.5 |
| MIA PaCa-2- Pancreatic carcinoma | 2.6 |
| CFPAC-1- Pancreatic ductal adenocarcinoma | 38.7 |
| PANC-1- Pancreatic epithelioid ductal carcinoma | 19.5 |
| T24- Bladder carcinma (transitional cell) | 9.0 |
| 5637- Bladder carcinoma | 10.5 |
| HT-1197- Bladder carcinoma | 4.8 |
| UM-UC-3- Bladder carcinma (transitional cell) | 13.3 |
| A204- Rhabdomyosarcoma | 15.2 |
| HT-1080- Fibrosarcoma | 11.9 |
| MG-63- Osteosarcoma | 7.3 |
| SK-LMS-1- Leiomyosarcoma (vulva) | 48.0 |
| SJRH30- Rhabdomyosarcoma (met to bone marrow) | 10.2 |
| A431- Epidermoid carcinoma | 12.2 |
| WM266-4- Melanoma | 21.9 |
| DU 145- Prostate carcinoma (brain metastasis) | 0.2 |
| MDA-MB-468- Breast adenocarcinoma | 5.6 |
| SCC-4- Squamous cell carcinoma of tongue | 0.3 |
| SCC-9- Squamous cell carcinoma of tongue | 0.3 |
| SCC-15- Squamous cell carcinoma of tongue | 0.2 |
| CAL 27- Squamous cell carcinoma of tongue | 19.9 |

TABLE AH
Panel 4D

| | Rel. |
|---|---|
| | Exp (%) |
| | Ag2100, |
| | Run |
| Tissue Name | 152800279 |
| Secondary Th1 act | 15.4 |
| Secondary Th2 act | 11.9 |
| Secondary Tr1 act | 15.6 |
| Secondary Th1 rest | 4.9 |
| Secondary Th2 rest | 3.3 |
| Secondary Tr1 rest | 6.0 |
| Primary Th1 act | 13.6 |
| Primary Th2 act | 12.0 |
| Primary Tr1 act | 22.2 |
| Primary Th1 rest | 100.0 |
| Primary Th2 rest | 37.9 |
| HUVEC IL-1beta | 12.2 |
| HUVEC IFN gamma | 16.6 |
| HUVEC TNF alpha + IFN gamma | 11.8 |
| HUVEC TNF alpha + IL4 | 11.4 |
| HUVEC IL-11 | 8.2 |
| Lung Microvascular EC none | 7.3 |
| Lung Microvascular EC TNFalpha + IL-1beta | 6.3 |
| Microvascular Dermal EC none | 23.3 |
| Microsvasular Dermal EC TNFalpha + IL-1beta | 10.5 |
| Bronchial epithelium TNFalpha + IL1beta | 0.6 |
| Small airway epithelium none | 1.6 |

TABLE AI
Panel CNS_1

| | Rel. |
|---|---|
| | Exp. (%) |
| | Ag2100, |
| | Run |
| Tissue Name | 171649357 |
| BA4 Control | 23.8 |
| BA4 Control2 | 19.1 |
| BA4 Alzheimer's2 | 7.3 |
| BA4 Parkinson's | 43.8 |
| BA4 Parkinson's2 | 60.7 |
| BA4 Huntington's | 23.3 |
| BA4 Huntington's2 | 14.7 |
| BA4 PSP | 13.8 |
| BA4 PSP2 | 26.2 |
| BA4 Depression | 15.4 |
| BA4 Depression2 | 17.0 |
| BA7 Control | 36.6 |
| BA7 Control2 | 17.4 |
| BA7 Alzheimer's2 | 11.3 |
| BA7 Parkinson's | 21.9 |
| BA7 Parkinson's2 | 36.1 |
| BA7 Huntington's | 56.3 |
| BA7 Huntington's2 | 45.1 |
| BA7 PSP | 44.4 |
| BA7 PSP2 | 17.6 |
| BA7 Depression | 8.5 |
| BA9 Control | 31.9 |
| BA9 Control2 | 34.4 |
| BA9 Alzheimer's | 8.0 |
| BA9 Alzheimer's2 | 20.0 |
| BA9 Parkinson's | 40.6 |
| BA9 Parkinson's2 | 31.4 |
| BA9 Huntington's | 41.5 |
| BA9 Huntington's2 | 21.8 |
| BA9 PSP | 17.8 |
| BA9 PSP2 | 8.2 |
| BA9 Depression | 10.5 |
| BA9 Depression2 | 16.2 |
| BA17 Control | 58.2 |
| BA17 Control2 | 41.8 |
| BA17 Alzheimer's2 | 27.0 |
| BA17 Parkinson's | 58.6 |
| BA17 Parkinson's2 | 69.3 |
| BA17 Huntington's | 44.4 |
| BA17 Huntington's2 | 31.9 |
| BA17 Depression | 13.6 |
| BA17 Depression2 | 100.0 |
| BA17 PSP | 35.4 |
| BA17 PSP2 | 18.3 |
| Sub Nigra Control | 11.6 |
| Sub Nigra Control2 | 5.0 |
| Sub Nigra Alzheimer's2 | 4.6 |
| Sub Nigra Parkinson's2 | 11.8 |
| Sub Nigra Huntington's | 16.0 |
| Sub Nigra Huntington's2 | 8.8 |
| Sub Nigra PSP2 | 1.7 |
| Sub Nigra Depression | 2.7 |
| Sub Nigra Depression2 | 8.0 |
| Glob Palladus Control | 8.4 |
| Glob Palladus Control2 | 10.8 |
| Glob Palladus Alzheimer's | 1.8 |
| Glob Palladus Alzheimer's2 | 8.3 |
| Glob Palladus Parkinson's | 51.1 |
| Glob Palladus Parkinson's2 | 12.9 |
| Glob Palladus PSP | 9.3 |
| Glob Palladus PSP2 | 9.9 |
| Glob Palladus Depression | 6.0 |
| Temp Pole Control | 9.8 |
| Temp Pole Control2 | 21.5 |
| Temp Pole Alzheimer's | 6.6 |
| Temp Pole Alzheimer's2 | 8.1 |
| Temp Pole Parkinson's | 33.0 |
| Temp Pole Parkinson's2 | 24.8 |
| Temp Pole Huntington's | 33.2 |
| Temp Pole PSP | 8.8 |
| Temp Pole PSP2 | 6.0 |
| Temp Pole Depression2 | 17.0 |
| Cing Gyr Control | 23.3 |
| Cing Gyr Control2 | 17.8 |
| Cing Gyr Alzheimer's | 7.3 |
| Cing Gyr Alzheimer's2 | 10.4 |
| Cing Gyr Parkinson's | 13.4 |
| Cing Gyr Parkinson's2 | 17.0 |
| Cing Gyr Huntington's | 28.3 |
| Cing Gyr Huntington's2 | 10.6 |
| Cing Gyr PSP | 7.2 |
| Cing Gyr PSP2 | 4.0 |
| Cing Gyr Depression | 6.9 |
| Cing Gyr Depression2 | 10.4 |

AI.05 chondrosarcoma Summary: Ag2100 Highest expression of this gene is detected in untreated serum starved chondrosarcoma cell line (SW1353) (CT=27). Interestingly, expression of this gene appears to be somewhat down regulated upon IL-1 treatment, a potent activator of pro-inflammatory cytokines and matrix metalloproteinases which participate in the destruction of cartilage observed in Osteoarthritis (OA). Modulation of the expression of this transcript in chondrocytes by either small molecules or antisense might be important for preventing the degeneration of cartilage observed in OA

Al_comprehensive panel_v1.0 Summary: Ag2100 Highest expression of this gene is detected in osteoarthritis (OA) bone (CTs=27-28). This gene is highly expressed in bone isolated from 5 different osteoarthritic (OA) patients, synovium in 3 out of 5 OA patients, but not in cartilege from OA patients nor in any tissues from rheumatoid arthritis (RA) patients or control samples. Thus, small molecule therapeutics designed against the protein encoded for by this gene could reduce or inhibit inflammation. Anti-sense therapeutics that would block the translation of the transcript and protein production could also inhibit inflammatory processes. These types of therapeutics could be important in the treatment of diseases such as osteoarthritis

CNS_neurodegeneration_v1.0 Summary: Ag2100 This panel confirms the expression of this gene at low levels in the brains of an independent group of individuals. However, no differential expression of this gene was detected between Alzheimer's diseased postmortem brains and those of non-demented controls in this experiment. Please see Panel 1.3D for a discussion of this gene in treatment of central nervous system disorders.

Panel 1.3D Summary: Ag2100 Expression of this gene is highest in cerebral cortex (CT=26.3). This gene is expressed at moderate levels in all the regions of the CNS including amygdala, cerebellum, hippocampus, substantia nigra, thalamus, spinal cord, and fetal brain. This gene encodes a protein with homology to citron-kinase. Citron-kinase (Citron-K) has been proposed by in vitro studies to be a crucial effector of Rho in regulation of cytokinesis. Citron-K is essential for cytokinesis in vivo in specific neuronal precursors and may play a fundamental role in specific human malformative syndromes of the CNS (Di Cunto et al., 2000, Neuron 28:115-127, PMID: 11086988). General inhibitors of the RHO/RAC-INTERACTING CITRON KINASE family disrupt endothelial tight junctions, suggesting that specific modulators of this brain-preferential family member could be useful in delivery of therapeutics across the blood brain barrier. These general inhibitors also influence intracellular calcium flux, which is a central component of many important neuronal processes, such as apoptosis, neurotransmitter release and signal transduction (Jezior et al., 2001, Br. J. Pharmacol. 134:78-87, PMID: 11522599; Walsh et al., 2001, Gastroenterology 121:566-579, PMID: 11522741). Thus, modulators of the function of the protein encoded by this gene may prove useful in the treatment of neurodegenerative disorders involving apoptosis, such as spinal muscular atrophy, Alzheimer's disease, Huntington's disease, Parkinson's disease, and others. Diseases involving neurotransmitters or signal transduction, such as schizophrenia, mania, stroke, epilepsy and depression may also benefit from agents that modulate the function of the this gene product.

This gene also shows moderate to low expression in several metabolic tissues including adrenal gland, pituitary gland, gastrointestinal tract, fetal heart, fetal skeletal muscle and fetal liver. Therefore, therapeutic modulation of the activity of this gene may prove useful in the treatment of endocrine/metabolically related diseases, such as obesity and diabetes.

Interestingly, expression of this gene is higher in fetal tissues (CTs=31) as compared to the corresponding adult liver, and skeletal muscle (CTs=37-40). This observation suggests that expression of this gene can be used to distinguish fetal from adult liver and skeletal muscle. In addition, the relative overexpression of this gene in fetal tissue suggests that the protein product may enhance liver and muscle growth or development in the fetus and thus may also act in a regenerative capacity in the adult. Therefore, therapeutic modulation of the protein encoded by this gene could be useful in treatment of liver and skeletal muscle related diseases.

Moderate levels of expression of this gene is also seen in cluster of cancer cell lines derived from pancreatic, gastric, colon, lung, liver, renal, breast, ovarian, prostate, melanoma and brain cancers. Thus, therapeutic modulation of the expression or function of this gene may be effective in the treatment of pancreatic, gastric, colon, lung, liver, renal, breast, ovarian, prostate, melanoma and brain cancers.

Panel 2.2 Summary: Ag2100 Expression of this gene is highest in a kidney cancer sample (CT=28). In addition, significant expression of this gene is also seen in a number of normal and cancer tissues including colon, lung, ovary, breast, kidney, thyroid, liver, bladder, and stomach. Interestingly, this gene is expressed at slightly higher levels in most of the tumors than in the normal matched tissue. Thus, expression of this gene could be used to distinguish between cancerous tissue and normal tissue. In addition, therapeutic modulation of this gene product, through the use of small molecule drugs or antibodies, might be of benefit in the treatment of cancer.

Panel 3D Summary: Ag2100 Expression of this gene is highest in a lung cancer cell line (CT=26). However, low to moderate expression is also seen in the majority of cancer cell lines on this panel, suggesting that this gene may play an important role in many cell types.

Panel 4D Summary: Ag2100 Highest expression of this gene is detected in resting primary Th1 cells (CT=24.5). Moderate to low levels of expression of this gene is seen in members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. Interestingly, this gene is highly induced in Ramos B cells treated with PMA and ionomycin, in non-transformed B cells and PBMC treated with PWM. All three of these observations are consistent with this gene being induced in B cells after activation. This gene product has homology to the RHO/RAC-interacting citron kinase. Thus citron kinase encoded by this gene may play an important role in T cell activation, by regulating TCR-mediated T cell spreading, chemotaxis and other chemokine responses and in apoptosis. Likewise, this putative kinase may also be important in B cell motility, antigen receptor mediated activation and apoptosis.

Small molecule therapeutics designed against the protein encoded for by this gene could reduce or inhibit inflammation. Anti-sense therapeutics that would block the translation of the transcript and protein production could also inhibit inflammatory processes. These types of therapeutics could be important in the treatment of diseases such as osteoarthritis. Likewise, these therapeutics could be important in the treatment of asthma, psoriasis, diabetes, and IBD, which require activated T cells, as well as diseases that involve B cell activation such as systemic lupus erythematosus.

Panel CNS_{—}1 Summary: Ag2100 This panel confirms the expression of this gene at low levels in the brains of an independent group of individuals. Please see Panel 1.3D for a discussion of this gene in treatment of central nervous system disorders.

B. CG117662-02: Renal Renin Precursor Like.

Expression of gene CG117662-02 was assessed using the primer-probe sets Ag2078 and Ag5185, described in Tables BA and BB. Results of the RTQ-PCR runs are shown in Tables BC, BD, BE, BF and BG.

TABLE BA
Probe Name Ag2078

| | | | Start | SEQ ID |
|---|---|---|---|---|
| Primers | Sequence | Length | Position | No |
| Forward | 5′-accttcaaagtcgtctttgaca-3′ | 22 | 292 | 252 |
| Probe | TET-5′-ctccaagtgcagccgtctctacactg-3′-TAMRA | 26 | 342 | 253 |
| Reverse | 5′cgaagagcttgtgatacacaca-3′ | 22 | 370 | 254 |

TABLE BB
Probe Name Ag5185

| | | | Start | SEQ ID |
|---|---|---|---|---|
| Primers | Sequence | Length | Position | No |
| Forward | 5′-ccgtgtctgtggggtcat-3′ | 18 | 491 | 255 |
| Probe | TET-5′-attggtagacaccggtgcatcctaca-3′-TAMRA | 26 | 540 | 256 |
| Reverse | 5′-tggagctggtagaacctgaga-3′ | 21 | 566 | 257 |

TABLE BC
CNS_neurodegeneration_v1.0

| | Rel. |
|---|---|
| | Exp. (%) |
| | Ag5185, |
| | Run |
| Tissue Name | 226559655 |
| AD 1 Hippo | 5.7 |
| AD 2 Hippo | 82.4 |
| AD 3 Hippo | 11.4 |
| AD 4 Hippo | 50.0 |
| AD 5 Hippo | 22.5 |
| AD 6 Hippo | 15.2 |
| Control 2 Hippo | 9.6 |
| Control 4 Hippo | 18.3 |
| Control (Path) 3 Hippo | 85.3 |
| AD 1 Temporal Ctx | 38.4 |
| AD 2 Temporal Ctx | 74.7 |
| AD 3 Temporal Ctx | 0.0 |
| AD 4 Temporal Ctx | 49.0 |
| AD 5 Inf Temporal Ctx | 31.6 |
| AD 5 Sup Temporal Ctx | 36.3 |
| AD 6 Inf Temporal Ctx | 55.5 |
| AD 6 Sup Temporal Ctx | 63.3 |
| Control 1 Temporal Ctx | 100.0 |
| Control 2 Temporal Ctx | 40.6 |
| Control 3 Temporal Ctx | 47.0 |
| Control 3 Temporal Ctx | 24.7 |
| Control (Path) 1 Temporal Ctx | 50.7 |
| Control (Path) 2 Temporal Ctx | 65.5 |
| Control (Path) 3 Temporal Ctx | 48.6 |
| Control (Path) 4 Temporal Ctx | 54.3 |
| AD 1 Occipital Ctx | 12.2 |
| AD 2 Occipital Ctx (Missing) | 0.0 |
| AD 3 Occipital Ctx | 18.8 |
| AD 4 Occipital Ctx | 19.8 |
| AD 5 Occipital Ctx | 12.1 |
| AD 6 Occipital Ctx | 25.0 |
| Control 1 Occipital Ctx | 26.2 |
| Control 2 Occipital Ctx | 3.6 |
| Control 3 Occipital Ctx | 40.6 |
| Control 4 Occipital Ctx | 20.9 |
| Control (Path) 1 Occipital Ctx | 39.2 |
| Control (Path) 2 Occipital Ctx | 18.3 |
| Control (Path) 3 Occipital Ctx | 0.0 |
| Control (Path) 4 Occipital Ctx | 0.0 |
| Control 1 Parietal Ctx | 46.7 |
| Control 2 Parietal Ctx | 0.0 |
| Control 3 Parietal Ctx | 12.2 |
| Control (Path) 1 Parietal Ctx | 65.5 |
| Control (Path) 2 Parietal Ctx | 23.8 |
| Control (Path) 3 Parietal Ctx | 0.0 |
| Control (Path) 4 Parietal Ctx | 57.4 |

TABLE BD
General_screening_panel_v1.5

| | Rel. |
|---|---|
| | Exp. (%) |
| | Ag5185, |
| | Run |
| Tissue Name | 228757766 |
| Adipose | 1.0 |
| Melanoma* Hs688(A).T | 0.2 |
| Melanoma* Hs688(B).T | 0.1 |
| Melanoma* M14 | 0.1 |
| Melanoma* LOXIMVI | 0.1 |
| Melanoma* SK-MEL-5 | 0.2 |
| Squamous cell carcinoma SCC-4 | 0.4 |
| Testis Pool | 8.4 |
| Prostate ca.* (bone met) PC-3 | 1.5 |
| Prostate Pool | 0.6 |
| Placenta | 3.0 |
| Uterus Pool | 1.5 |
| Ovarian ca. OVCAR-3 | 0.9 |
| Ovarian ca. SK-OV-3 | 0.2 |
| Ovarian ca. OVCAR-4 | 0.7 |
| Ovarian ca. OVCAR-5 | 4.7 |
| Ovarian ca. IGROV-1 | 0.0 |
| Ovarian ca. OVCAR-8 | 0.2 |
| Ovary | 6.7 |
| Breast ca. MCF-7 | 0.5 |
| Breast ca. MDA-MB-231 | 0.6 |
| Breast ca. BT 549 | 0.2 |
| Breast ca. T47D | 2.1 |
| Breast ca. MDA-N | 0.0 |
| Breast Pool | 5.0 |
| Trachea | 1.0 |
| Lung | 22.1 |
| Fetal Lung | 0.6 |
| Lung ca. NCI-N417 | 0.4 |
| Lung ca. LX-1 | 0.3 |
| Lung ca. NCI-H146 | 0.0 |
| Lung ca. SHP-77 | 0.1 |
| Lung ca. A549 | 0.0 |
| Lung ca. NCI-H526 | 0.5 |
| Lung ca. NCI-H23 | 1.4 |
| Lung ca. NCI-H460 | 2.0 |
| Lung ca. HOP-62 | 0.1 |
| Lung ca. NCI-H522 | 0.6 |
| Liver | 1.0 |
| Fetal Liver | 1.0 |
| Liver ca. HepG2 | 10.0 |
| Kidney Pool | 4.2 |
| Fetal Kidney | 100.0 |
| Renal ca. 786-0 | 0.0 |
| Renal ca. A498 | 0.0 |
| Renal ca. ACHN | 0.2 |
| Renal ca. UO-31 | 0.3 |
| Renal ca. TK-10 | 0.0 |
| Bladder | 0.5 |
| Gastric ca. (liver met.) NCI-N87 | 1.1 |
| Gastric ca. KATO III | 0.3 |
| Colon ca. SW-948 | 18.2 |
| Colon ca. SW480 | 0.6 |
| Colon ca.* (SW480 met) SW620 | 0.5 |
| Colon ca. HT29 | 1.6 |
| Colon ca. HCT-116 | 0.5 |
| Colon ca. CaCo-2 | 0.2 |
| Colon cancer tissue | 2.6 |
| Colon ca. SW1116 | 0.1 |
| Colon ca. Colo-205 | 0.0 |
| Colon ca. SW-48 | 0.8 |
| Colon Pool | 4.7 |
| Small Intestine Pool | 4.0 |
| Stomach Pool | 2.3 |
| Bone Marrow Pool | 2.5 |
| Fetal Heart | 0.2 |
| Heart Pool | 1.6 |
| Lymph Node Pool | 12.6 |
| Fetal Skeletal Muscle | 0.3 |
| Skeletal Muscle Pool | 0.4 |
| Spleen Pool | 0.1 |
| Thymus Pool | 3.8 |
| CNS cancer (glio/astro) U87-MG | 0.0 |
| CNS cancer (glio/astro) U-118-MG | 0.1 |
| CNS cancer (neuro; met) SK-N-AS | 0.0 |
| CNS cancer (astro) SF-539 | 0.0 |
| CNS cancer (astro) SNB-75 | 0.0 |
| CNS cancer (glio) SNB-19 | 0.3 |
| CNS cancer (glio) SF-295 | 0.5 |
| Brain (Amygdala) Pool | 0.4 |
| Brain (cerebellum) | 0.5 |
| Brain (fetal) | 0.0 |
| Brain (Hippocampus) Pool | 0.2 |
| Cerebral Cortex Pool | 0.3 |
| Brain (Substantia nigra) Pool | 0.3 |
| Brain (Thalamus) Pool | 0.6 |
| Brain (whole) | 0.8 |
| Spinal Cord Pool | 0.5 |
| Adrenal Gland | 2.6 |
| Pituitary gland Pool | 0.6 |
| Salivary Gland | 0.5 |
| Thyroid (female) | 0.1 |
| Pancreatic ca. CAPAN2 | 0.2 |
| Pancreas Pool | 4.9 |

TABLE BE
Panel 1.3D

| | Rel. | Rel. | Rel. |
|---|---|---|---|
| | Exp. (%) | Exp. (%) | Exp. (%) |
| | Ag2078, | Ag2078, | Ag2078, |
| | Run | Run | Run |
| Tissue Name | 165626684 | 165627496 | 165678122 |
| Liver adenocarcinoma | 0.0 | 0.1 | 0.1 |
| Pancreas | 0.0 | 0.0 | 0.0 |
| Pancreatic ca. CAPAN 2 | 0.0 | 0.0 | 0.2 |
| Adrenal gland | 0.5 | 0.5 | 0.3 |
| Thyroid | 0.0 | 0.0 | 0.0 |
| Salivary gland | 0.0 | 0.1 | 0.0 |
| Pituitary gland | 0.0 | 0.2 | 0.0 |
| Brain (fetal) | 0.0 | 0.0 | 0.0 |
| Brain (whole) | 0.0 | 0.0 | 0.1 |
| Brain (amygdala) | 0.1 | 0.0 | 0.0 |
| Brain (cerebellum) | 0.1 | 0.0 | 0.1 |
| Brain (hippocampus) | 0.0 | 0.3 | 0.0 |
| Brain (substantia | 0.0 | 0.0 | 0.1 |
| nigra) | | | |
| Brain (thalamus) | 0.1 | 0.0 | 0.1 |
| Cerebral Cortex | 0.0 | 0.0 | 0.2 |
| Spinal cord | 0.0 | 0.0 | 0.0 |
| glio/astro U87-MG | 0.0 | 0.0 | 0.0 |
| glio/astro U-118-MG | 0.0 | 0.0 | 0.0 |
| astrocytoma SW1783 | 0.0 | 0.0 | 0.1 |
| neuro*; met SK-N-AS | 0.0 | 0.1 | 0.0 |
| astrocytoma SF-539 | 0.0 | 0.0 | 0.0 |
| astrocytoma SNB-75 | 0.0 | 0.0 | 0.2 |
| glioma SNB-19 | 0.0 | 0.0 | 0.0 |
| glioma U251 | 0.0 | 0.0 | 0.0 |
| glioma SF-295 | 0.0 | 0.0 | 0.0 |
| Heart (fetal) | 0.0 | 0.0 | 0.0 |
| Heart | 0.0 | 0.0 | 0.0 |
| Skeletal muscle | 0.0 | 0.0 | 0.0 |
| (fetal) | | | |
| Skeletal muscle | 0.0 | 0.0 | 0.0 |
| Bone marrow | 0.0 | 0.0 | 0.0 |
| Thymus | 0.0 | 0.0 | 0.0 |
| Spleen | 0.0 | 0.0 | 0.0 |
| Lymph node | 0.0 | 0.1 | 0.0 |
| Colorectal | 0.0 | 0.0 | 0.0 |
| Stomach | 0.0 | 0.0 | 0.1 |
| Small intestine | 0.1 | 0.0 | 0.0 |
| Colon ca. SW480 | 0.0 | 0.0 | 0.0 |
| Colon ca.* SW620 | 0.0 | 0.0 | 0.0 |
| (SW480 met) | | | |
| Colon ca. HT29 | 0.2 | 0.3 | 0.3 |
| Colon ca. HCT-116 | 0.0 | 0.0 | 0.0 |
| Colon ca. CaCo-2 | 0.0 | 0.0 | 0.0 |
| Colon ca. tissue | 0.2 | 0.1 | 0.5 |
| (ODO3866) | | | |
| Colon ca. HCC-2998 | 0.1 | 0.3 | 0.1 |
| Gastric ca.* (liver | 0.0 | 0.1 | 0.0 |
| met) NCI-N87 | | | |
| Bladder | 0.0 | 0.0 | 0.0 |
| Trachea | 0.1 | 0.0 | 0.0 |
| Kidney | 11.2 | 10.8 | 8.7 |
| Kidney (fetal) | 100.0 | 100.0 | 100.0 |
| Renal ca. 786-0 | 0.0 | 0.0 | 0.0 |
| Renal ca. A498 | 0.0 | 0.0 | 0.1 |
| Renal ca. RXF 393 | 0.0 | 0.0 | 0.0 |
| Renal ca. ACHN | 0.0 | 0.0 | 0.0 |
| Renal ca. UO-31 | 0.0 | 0.0 | 0.1 |
| Renal ca. TK-10 | 0.0 | 0.1 | 0.0 |
| Liver | 0.3 | 0.3 | 0.0 |
| Liver (fetal) | 0.6 | 0.7 | 0.5 |
| Liver ca. | 0.0 | 0.0 | 0.0 |
| (hepatoblast) HepG2 | | | |
| Lung | 0.0 | 0.0 | 0.1 |
| Lung (fetal) | 0.1 | 0.1 | 0.0 |
| Lung ca. (small | 0.0 | 0.0 | 0.0 |
| cell) LX-1 | | | |
| Lung ca. (small | 0.0 | 0.0 | 0.0 |
| cell) NCI-H69 | | | |
| Lung ca. (s. cell | 0.0 | 0.1 | 0.0 |
| var.) SHP-77 | | | |
| Lung ca. (large | 0.0 | 0.0 | 0.0 |
| cell) NCI-H460 | | | |
| Lung ca. (non-sm. | 0.0 | 0.0 | 0.0 |
| cell) A549 | | | |
| Lung ca. (non-s. | 0.0 | 0.0 | 0.0 |
| cell) NCI-H23 | | | |
| Lung ca. (non-s. | 0.1 | 0.0 | 0.0 |
| cell) HOP-62 | | | |
| Lung ca. (non-s. | 0.0 | 0.0 | 0.0 |
| cl) NCI-H522 | | | |
| Lung ca. (squam.) SW | 0.1 | 0.1 | 0.0 |
| 900 | | | |
| Lung ca. (squam.) | 0.0 | 0.0 | 0.0 |
| NCI-H596 | | | |
| Mammary gland | 0.2 | 0.2 | 0.1 |
| Breast ca.* (pl. | 0.0 | 0.0 | 0.1 |
| ef) MCF-7 | | | |
| Breast ca.* (pl. | 0.1 | 0.0 | 0.0 |
| ef) MDA-MB-231 | | | |
| Breast ca.* (pl. | 0.1 | 0.0 | 0.0 |
| ef) T47D | | | |
| Breast ca. BT-549 | 0.0 | 0.0 | 0.0 |
| Breast ca. MDA-N | 0.0 | 0.0 | 0.0 |
| Ovary | 0.6 | 0.8 | 0.6 |
| Ovarian ca. OVCAR-3 | 0.1 | 0.1 | 0.0 |
| Ovarian ca. OVCAR-4 | 0.0 | 0.1 | 0.0 |
| Ovarian ca. OVCAR-5 | 0.2 | 0.2 | 0.1 |
| Ovarian ca. OVCAR-8 | 0.0 | 0.0 | 0.0 |
| Ovarian ca. IGROV-1 | 0.0 | 0.0 | 0.0 |
| Ovarian ca.* (ascites) | 0.0 | 0.0 | 0.0 |
| SK-OV-3 | | | |
| Uterus | 1.7 | 1.1 | 1.1 |
| Placenta | 0.7 | 1.2 | 0.7 |
| Prostate | 0.1 | 0.0 | 0.1 |
| Prostate ca.* | 0.2 | 0.2 | 0.0 |
| (bone met) PC-3 | | | |
| Testis | 0.2 | 0.1 | 0.2 |
| Melanoma Hs688(A).T | 0.0 | 0.0 | 0.0 |
| Melanoma* (met) | 0.0 | 0.0 | 0.0 |
| Hs688(B).T | | | |
| Melanoma UACC-62 | 0.0 | 0.0 | 0.0 |
| Melanoma M14 | 0.0 | 0.0 | 0.0 |
| Melanoma LOX IMVI | 0.0 | 0.0 | 0.1 |
| Melanoma* (met) | 0.0 | 0.0 | 0.0 |
| SK-MEL-5 | | | |
| Adipose | 0.0 | 0.2 | 0.0 |

TABLE BF
Panel 4D

| | Rel. |
|---|---|
| | Exp. (%) |
| | Ag2078, |
| | Run |
| Tissue Name | 161905846 |
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 0.8 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.1 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 0.1 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 0.0 |
| EOL-1 dbcAMP | 0.0 |
| EOL-1 dbcAMP PMA/ionomycin | 0.2 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 0.4 |
| HUVEC starved | 0.2 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.2 |
| Lung Microvascular EC TNFalpha + IL-1beta | 0.2 |
| Microvascular Dermal EC none | 0.3 |
| Microsvasular Dermal EC TNFalpha + IL-1beta | 0.1 |
| Bronchial epithelium TNFalpha + IL1beta | 0.1 |
| Small airway epithelium none | 0.0 |
| Small airway epithelium TNFalpha + IL-1beta | 0.5 |
| Coronery artery SMC rest | 0.1 |
| Coronery artery SMC TNFalpha + IL-1beta | 0.1 |
| Astrocytes rest | 0.0 |
| Astrocytes TNFalpha + IL-1beta | 0.1 |
| KU-812 (Basophil) rest | 0.0 |
| KU-812 (Basophil) PMA/ionomycin | 0.0 |
| CCD1106 (Keratinocytes) none | 0.0 |
| CCD1106 (Keratinocytes) TNFalpha + IL-1beta | 0.0 |
| Liver cirrhosis | 0.4 |
| Lupus kidney | 3.9 |
| NCI-H292 none | 1.3 |
| NCI-H292 IL-4 | 0.5 |
| NCI-H292 IL-9 | 1.9 |
| NCI-H292 IL-13 | 0.3 |
| NCI-H292 IFN gamma | 1.0 |
| HPAEC none | 0.0 |
| HPAEC TNF alpha + IL-1beta | 0.0 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 5.9 |
| Dermal fibroblast CCD1070 TNF alpha | 4.5 |
| Dermal fibroblast CCD1070 IL-1 beta | 3.1 |
| Dermal fibroblast IFN gamma | 0.0 |
| Dermal fibroblast IL-4 | 0.0 |
| IBD Colitis 2 | 0.0 |
| IBD Crohn's | 0.0 |
| Colon | 0.0 |
| Lung | 0.2 |
| Thymus | 100.0 |
| Kidney | 0.4 |

TABLE BG
Panel 5D

| | Rel. |
|---|---|
| | Exp. (%) |
| | Ag2078, |
| | Run |
| Tissue Name | 168095527 |
| 97457_Patient-02go_adipose | 11.7 |
| 97476_Patient-07sk_skeletal muscle | 0.0 |
| 97477_Patient-07ut uterus | 2.8 |
| 97478_Patient-07pl_placenta | 12.9 |
| 97481_Patient-08sk_skeletal muscle | 0.0 |
| 97482_Patient-08ut_uterus | 22.8 |
| 97483_Patient-08pl_placenta | 4.5 |
| 97486_Patient-09sk_skeletal muscle | 0.0 |
| 97487_Patient-09ut_uterus | 0.0 |
| 97488_Patient-09pl_placenta | 2.7 |
| 97492_Patient-10ut_uterus | 100.0 |
| 97493_Patient-10pl_placenta | 5.4 |
| 97495_Patient-11go_adipose | 6.0 |
| 97496_Patient-11sk_skeletal muscle | 0.0 |
| 97497 Patient-11ut_uterus | 12.8 |
| 97498_Patient-11pl_placenta | 8.5 |
| 97500_Patient-12go_adipose | 87.1 |
| 97501_Patient-12sk_skeletal muscle | 0.0 |
| 97502_Patient-12ut_uterus | 4.6 |
| 97503_Patient-12pl_placenta | 8.0 |
| 94721_Donor 2 U - A_Mesenchymal Stem Cells | 0.0 |
| 94722_Donor 2 U - B_Mesenchymal Stem Cells | 0.0 |
| 94723_Donor 2 U - C_Mesenchymal Stem Cells | 0.0 |
| 94709_Donor 2 AM - A_adipose | 0.0 |
| 94710_Donor 2 AM - B_adipose | 0.0 |
| 94711_Donor 2 AM - C_adipose | 0.0 |
| 94712_Donor 2 AD - A_adipose | 1.0 |
| 94713_Donor 2 AD - B_adipose | 0.0 |
| 94714_Donor 2 AD - C_adipose | 0.0 |
| 94742_Donor 3 U - A_Mesenchymal Stem Cells | 0.0 |
| 94743_Donor 3 U - B_Mesenchymal Stem Cells | 0.0 |
| 94730_Donor 3 AM - A_adipose | 0.9 |
| 94731_Donor 3 AM - B_adipose | 0.0 |
| 94732_Donor 3 AM - C_adipose | 0.0 |
| 94733_Donor 3 AD - A_adipose | 0.0 |
| 94734_Donor 3 AD - B_adipose | 0.0 |
| 94735_Donor 3 AD - C_adipose | 0.0 |
| 77138_Liver_HepG2untreated | 0.0 |
| 73556_Heart_Cardiac stromal cells (primary) | 0.0 |
| 81735_Small Intestine | 0.0 |
| 72409_Kidney_Proximal Convoluted Tubule | 0.0 |
| 82685_Small intestine_Duodenum | 0.0 |
| 90650_Adrenal_Adrenocortical adenoma | 0.0 |
| 72410_Kidney_HRCE | 1.1 |
| 72411_Kidney_HRE | 5.3 |
| 73139_Uterus_Uterine smooth muscle cells | 2.4 |

CNS_neurodegeneration_v1.0 Summary: Ag5185 Low levels of expression of this gene is seen in control temporal cortex and in a hippocampus sample from an Alzheimer patient (CTs=34.6-34.9). Therefore, therapeutic modulation of this gene may be useful in the neurological disorders including seizure and memory related diseases.

General_screening_panel_v1.5 Summary: Ag5185 Highest expression of this gene is detected in fetal kidney (CT=26.7). Interestingly, expression of this gene is higher in fetal as compared to adult kidney (CT=31). This observation suggests that expression of this gene can be used to distinguish fetal from adult kidney and also from other samples in this panel. In addition, the relative overexpression of this gene in fetal tissue suggests that the protein product may enhance kidney growth or development in the fetus and thus may also act in a regenerative capacity in the adult. Therefore, therapeutic modulation of the protein encoded by this gene could be useful in treatment of kidney related diseases including lupus and glomerulonephritis.

Moderate to low levels of expression of this gene is also seen in tissues with metabolic/endocrine functions such as pancreas, adiposes, adrenal and pituitary glands, heart, skeletal muscle, and gastrointestinal tract. Therefore, therapeutic modulation of the activity of this gene may prove useful in the treatment of endocrine/metabolically related diseases, such as obesity and diabetes.

Moderate to low levels of expression of this gene is also seen in a number of cancer cell lines derived from colon, lung, and ovarian cancer. Therefore, therapeutic modulation of this gene may be useful in the treatment of colon, lung and ovarian cancers.

Panel 1.3D Summary: Ag2078 Three experiments with same probe-primer sets are in excellent agreement. Highest expression of this gene is seen in fetal kidney (CTs=26-27.8), with lower expression in the adult lung. This pattern correlates to the expression seen in panel 1.5. Please see panel 1.5 for further discussion of this gene.

Panel 4D Summary: Ag2078 Highest expression of this gene is detected in thymus (CT=27.3). This gene or its protein product may thus play an important role in T cell development. Small molecule therapeutics, or antibody therapeutics designed against the protein encoded for by this gene could be utilized to modulate immune function (T cell development) and be important for organ transplant, AIDS treatment or post chemotherapy immune reconstitiution.

Moderate to low levels of expression of this gene is also seen in lupus kidney, resting and cytokine activated mucoepidermoid NCI-H292 cells and dermal fibroblasts. Therefore, therapeutic modulation of this gene may be useful in the treatment of chronic obstructive pulmonary disease, asthma, allergy, emphysema, lupus kidney and skin disorders, including psoriasis.

Panel 5D Summary: Ag2078 Highest expression of this gene is detected in uterus and adipose of diabetic patients on insulin (CT=30.9-31). In addition, moderate to low levels of expression of this gene is also seen in uterus and placenta. Therefore, therapeutic modulation of this gene may be useful in the treatment of obesity and diabetes.

C. CG118051-02: ALDH8 Splice Variant, Submitted to Study DDSMT on Sep. 26, 2001 by Saguo; Classification Type=Finished In-Silico; Novelty=Update-Variants; ORF Start=407, ORF Stop=1436, Frame=2; 1586 bp.

Expression of gene CG118051-02 was assessed using the primer-probe set Ag3729, described in Table CA. Results of the RTQ-PCR runs are shown in Tables CB and CC.

TABLE CA
Probe Name Ag3729

| | | | Start | SEQ ID |
|---|---|---|---|---|
| Primers | | Length | Position | No |
| Forward | 5′-ttcaagaaaacaagcagcttct-3′ | 22 | 273 | 258 |
| Probe | TET-5′-cccaggacctgcataagccagct-3′-TAMRA | 23 | 309 | 259 |
| Reverse | 5′-ctcagatatqtctgcctcgaa-3′ | 21 | 332 | 260 |

TABLE CB
Panel 2.2

| | Rel. | Rel. |
|---|---|---|
| | Exp. (%) | Exp. (%) |
| | Ag3729, | Ag3729, |
| | Run | Run |
| Tissue Name | 174441818 | 259034396 |
| Normal Colon | 0.4 | 0.3 |
| Colon cancer (OD06064) | 1.4 | 1.0 |
| Colon Margin (OD06064) | 0.0 | 0.0 |
| Colon cancer (OD06159) | 0.2 | 0.1 |
| Colon Margin (OD06159) | 0.0 | 0.0 |
| Colon cancer (OD06297-04) | 0.0 | 0.0 |
| Colon Margin (OD06297-05) | 0.0 | 0.0 |
| CC Gr.2 ascend colon (ODO3921) | 1.1 | 0.8 |
| CC Margin (ODO3921) | 0.0 | 0.0 |
| Colon cancer metastasis (OD06104) | 0.2 | 0.1 |
| Lung Margin (OD06104) | 0.0 | 0.0 |
| Colon mets to lung (OD04451-01) | 0.2 | 0.2 |
| Lung Margin (OD04451-02) | 0.0 | 0.0 |
| Normal Prostate | 2.3 | 1.8 |
| Prostate Cancer (OD04410) | 2.2 | 1.6 |
| Prostate Margin (OD04410) | 5.1 | 3.8 |
| Normal Ovary | 0.7 | 0.3 |
| Ovarian cancer (OD06283-03) | 2.5 | 1.7 |
| Ovarian Margin (OD06283-07) | 0.0 | 0.0 |
| Ovarian Cancer 064008 | 1.0 | 0.6 |
| Ovarian cancer (OD06145) | 0.4 | 0.3 |
| Ovarian Margin (OD06145) | 0.5 | 0.3 |
| Ovarian cancer (OD06455-03) | 0.9 | 0.5 |
| Ovarian Margin (OD06455-07) | 0.0 | 0.0 |
| Normal Lung | 0.0 | 0.0 |
| Invasive poor diff. lung adeno | 9.2 | 7.5 |
| (ODO4945-01 | | |
| Lung Margin (ODO4945-03) | 0.0 | 0.0 |
| Lung Malignant Cancer (OD03126) | 0.5 | 0.4 |
| Lung Margin (OD03126) | 0.4 | 0.3 |
| Lung Cancer (OD05014A) | 0.0 | 0.0 |
| Lung Margin (OD05014B) | 0.8 | 0.6 |
| Lung cancer (OD06081) | 44.8 | 0.3 |
| Lung Margin (OD06081) | 0.0 | 0.0 |
| Lung Cancer (OD04237-01) | 3.1 | 2.6 |
| Lung Margin (OD04237-02) | 0.4 | 0.3 |
| Ocular Melanoma Metastasis | 0.0 | 0.0 |
| Ocular Melanoma Margin (Liver) | 0.0 | 0.0 |
| Melanoma Metastasis | 0.0 | 0.0 |
| Melanoma Margin (Lung) | 0.3 | 0.2 |
| Normal Kidney | 0.0 | 0.0 |
| Kidney Ca, Nuclear grade 2 (OD04338) | 1.5 | 1.2 |
| Kidney Margin (OD04338) | 0.4 | 0.3 |
| Kidney Ca Nuclear grade 1/2 (OD04339) | 0.0 | 0.0 |
| Kidney Margin (OD04339) | 0.0 | 0.0 |
| Kidney Ca, Clear cell type (OD04340) | 0.0 | 0.0 |
| Kidney Margin (OD04340) | 0.4 | 0.3 |
| Kidney Ca, Nuclear grade 3 (OD04348) | 0.0 | 0.0 |
| Kidney Margin (OD04348) | 0.0 | 0.0 |
| Kidney malignant cancer (OD06204B) | 0.0 | 0.0 |
| Kidney normal adjacent tissue | 0.0 | 0.0 |
| (OD06204E) | | |
| Kidney Cancer (OD04450-01) | 0.0 | 0.0 |
| Kidney Margin (OD04450-03) | 1.3 | 0.9 |
| Kidney Cancer 8120613 | 0.0 | 0.0 |
| Kidney Margin 8120614 | 0.0 | 0.0 |
| Kidney Cancer 9010320 | 0.5 | 0.3 |
| Kidney Margin 9010321 | 1.8 | 1.4 |
| Kidney Cancer 8120607 | 0.0 | 0.0 |
| Kidney Margin 8120608 | 1.0 | 0.8 |
| Normal Uterus | 0.0 | 0.0 |
| Uterine Cancer 064011 | 1.8 | 1.2 |
| Normal Thyroid | 0.0 | 0.0 |
| Thyroid Cancer 064010 | 0.0 | 0.0 |
| Thyroid Cancer A302152 | 0.0 | 0.0 |
| Thyroid Margin A302153 | 0.0 | 0.0 |
| Normal Breast | 9.2 | 6.5 |
| Breast Cancer (OD04566) | 17.4 | 12.9 |
| Breast Cancer 1024 | 100.0 | 100.0 |
| Breast Cancer (OD04590-01) | 3.9 | 2.5 |
| Breast Cancer Mets (OD04590-03) | 1.2 | 0.9 |
| Breast Cancer Metastasis (OD04655-05) | 48.6 | 34.4 |
| Breast Cancer 064006 | 2.4 | 2.1 |
| Breast Cancer 9100266 | 55.1 | 43.8 |
| Breast Margin 9100265 | 14.7 | 10.8 |
| Breast Cancer A209073 | 32.1 | 24.5 |
| Breast Margin A2090734 | 9.1 | 6.4 |
| Breast cancer (OD06083) | 69.7 | 61.6 |
| Breast cancer node metastasis | 28.5 | 23.3 |
| (OD06083) | | |
| Normal Liver | 0.0 | 0.0 |
| Liver Cancer 1026 | 0.0 | 0.0 |
| Liver Cancer 1025 | 0.8 | 0.6 |
| Liver Cancer 6004-T | 0.2 | 0.1 |
| Liver Tissue 6004-N | 0.4 | 0.3 |
| Liver Cancer 6005-T | 0.0 | 0.0 |
| Liver Tissue 6005-N | 0.0 | 0.0 |
| Liver Cancer 064003 | 0.0 | 0.0 |
| Normal Bladder | 0.0 | 0.0 |
| Bladder Cancer 1023 | 3.2 | 2.3 |
| Bladder Cancer A302173 | 4.5 | 3.2 |
| Normal Stomach | 0.0 | 0.0 |
| Gastric Cancer 9060397 | 0.5 | 0.3 |
| Stomach Margin 9060396 | 2.1 | 1.4 |
| Gastric Cancer 9060395 | 2.5 | 1.7 |
| Stomach Margin 9060394 | 1.8 | 1.1 |
| Gastric Cancer 064005 | 0.0 | 0.0 |

TABLE CC
Panel 4.1D

| | Rel. |
|---|---|
| | Exp. (%) |
| | Ag3729, |
| | Run |
| Tissue Name | 170222887 |
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 0.0 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.0 |
| PBMC PHA-L | 0.0 |
| Ramos (B cell) none | 7.4 |
| Ramos (B cell) ionomycin | 3.1 |
| B lymphocytes PWM | 0.0 |
| B lymphocytes CD40L and IL-4 | 0.0 |
| EOL-1 dbcAMP | 0.0 |
| EOL-1 dbcAMP PMA/ionomycin | 0.0 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 26.8 |
| Small airway epithelium none | 25.5 |
| Small airway epithelium TNFalpha + IL-1beta | 46.7 |
| Coronery artery SMC rest | 0.0 |
| Coronery artery SMC TNFalpha + IL-1beta | 0.0 |
| Astrocytes rest | 0.0 |
| Astrocytes TNFalpha + IL-1beta | 0.0 |
| KU-812 (Basophil) rest | 0.0 |
| KU-812 (Basophil) PMA/ionomycin | 0.0 |
| CCD1106 (Keratinocytes) none | 0.0 |
| CCD1106 (Keratinocytes) TNFalpha + IL-1beta | 6.7 |
| Liver cirrhosis | 0.0 |
| NCI-H292 none | 100.0 |
| NCI-H292 IL-4 | 55.9 |
| NCI-H292 IL-9 | 82.9 |
| NCI-H292 IL-13 | 58.2 |
| NCI-H292 IFN gamma | 60.3 |
| HPAEC none | 0.0 |
| HPAEC TNF alpha + IL-1 beta | 0.0 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 0.0 |
| Dermal fibroblast IL-4 | 0.0 |
| Dermal Fibroblasts rest | 0.0 |
| Neutrophils TNFa + LPS | 0.0 |
| Neutrophils rest | 0.0 |
| Colon | 0.0 |
| Lung | 6.3 |
| Thymus | 7.8 |
| Kidney | 2.6 |

CNS_neurodegeneration_v1.0 Summary: Ag3729 Expression of this gene is low/undetectable in all samples on this panel (CTs>35).

Panel 2.2 Summary: Ag3729 Two experiments with same probe-primer sets are in good agreement. Highest expression of this gene is seen in breast cancer (CTs=27-29). Thus, expression of this gene could be used to differentiate between the breast cancer samples and other samples on this panel.

In addition, moderate expression of this gene is also seen in cancer samples derived from colon, breast, ovarian, lung, bladder, kidney and uterine cancers. Interestingly, expression of gene higher cancer compared to the corresponding normal adjacent tissue. Thus, expression of this gene may be used as diagnostic marker to detect the presence of colon, breast, ovarian, lung, bladder, kidney and uterine cancers and also, therapeutic modulation of the expression or function of this gene may be effective in the treatment of these cancers.

Panel 4.1D Summary: Ag3729 Expression of this gene is restricted to a few samples, with highest expression is seen in untreated NCI-H292 cells (CT=31.4). The gene is also expressed in a cluster of treated and untreated samples derived from the NCI-H292 cell line, a human airway epithelial cell line that produces mucins. Mucus overproduction is an important feature of bronchial asthma and chronic obstructive pulmonary disease samples. Interestingly, the transcript is also expressed at lower but still significant levels in small airway and bronchial epithelium treated with IL-1 beta and TNF-alpha and untreated small airway epithelium. The expression of the transcript in this mucoepidermoid cell line that is often used as a model for airway epithelium (NCI-H292 cells) suggests that this transcript may be important in the proliferation or activation of airway epithelium. Therefore, therapeutics designed with the protein encoded by the transcript may reduce or eliminate symptoms caused by inflammation in lung epithelia in chronic obstructive pulmonary disease, asthma, allergy, and emphysema.

D. CG140468-02: Serine/Threonine-Protein Kinase Pak 1.

Expression of gene CG140468-02 was assessed using the primer-probe set Ag7054, described in Table DA. Results of the RTQ-PCR runs are shown in Table DB. Please note that CG140468-02 represents a full-length physical clone.

TABLE DA
Probe Name Ag7054

| | | | Start | SEQ ID |
|---|---|---|---|---|
| Primers | Sequence | Length | Position | No |
| Forward | 5′-ggtttgagaagattgccaagc-3′ | 21 | 819 | 261 |
| Probe | TET-5′-cctcactccactgattgctgcagctaa-3′-TAMRA | 27 | 850 | 262 |
| Reverse | 5′-ctggggtgagtgtggttttag-3′ | 21 | 898 | 263 |

TABLE DB
General_screening_panel_v1.6

| | Rel. |
|---|---|
| | Exp. (%) |
| | Ag7054, |
| | Run |
| Tissue Name | 282273878 |
| Adipose | 3.6 |
| Melanoma* Hs688(A).T | 7.3 |
| Melanoma* Hs688(B).T | 6.6 |
| Melanoma* M14 | 13.3 |
| Melanoma* LOXIMVI | 21.6 |
| Melanoma* SK-MEL-5 | 8.1 |
| Squamous cell carcinoma SCC-4 | 7.7 |
| Testis Pool | 5.6 |
| Prostate ca.* (bone met) PC-3 | 3.3 |
| Prostate Pool | 8.0 |
| Placenta | 9.5 |
| Uterus Pool | 2.4 |
| Ovarian ca. OVCAR-3 | 100.0 |
| Ovarian ca. SK-OV-3 | 16.4 |
| Ovarian ca. OVCAR-4 | 3.3 |
| Ovarian ca. OVCAR-5 | 35.1 |
| Ovarian ca. IGROV-1 | 5.3 |
| Ovarian ca. OVCAR-8 | 8.4 |
| Ovary | 5.1 |
| Breast ca. MCF-7 | 2.2 |
| Breast ca. MDA-MB-231 | 11.8 |
| Breast ca. BT 549 | 4.2 |
| Breast ca. T47D | 7.7 |
| Breast ca. MDA-N | 5.8 |
| Breast Pool | 8.8 |
| Trachea | 7.7 |
| Lung | 4.1 |
| Fetal Lung | 7.9 |
| Lung ca. NCI-N417 | 7.9 |
| Lung ca. LX-1 | 19.9 |
| Lung ca. NCI-H146 | 3.5 |
| Lung ca. SHP-77 | 5.8 |
| Lung ca. A549 | 8.8 |
| Lung ca. NCI-H526 | 3.5 |
| Lung ca. NCI-H23 | 11.0 |
| Lung ca. NCI-H460 | 1.0 |
| Lung ca. HOP-62 | 3.5 |
| Lung ca. NCI-H522 | 20.7 |
| Liver | 0.7 |
| Fetal Liver | 9.1 |
| Liver ca. HepG2 | 0.5 |
| Kidney Pool | 11.3 |
| Fetal Kidney | 16.0 |
| Renal ca. 786-0 | 9.9 |
| Renal ca. A498 | 4.4 |
| Renal ca. ACHN | 6.9 |
| Renal ca. UO-31 | 13.5 |
| Renal ca.TK-10 | 10.7 |
| Bladder | 9.0 |
| Gastric ca. (liver met.) NCI-N87 | 30.6 |
| Gastric ca. KATO III | 49.3 |
| Colon ca. SW-948 | 7.8 |
| Colon ca. SW480 | 2.5 |
| Colon ca.* (SW480 met) SW620 | 11.8 |
| Colon ca. HT29 | 22.2 |
| Colon ca. HCT-116 | 19.1 |
| Colon ca. CaCo-2 | 34.6 |
| Colon cancer tissue | 9.0 |
| Colon ca. SW1116 | 4.5 |
| Colon ca. Colo-205 | 10.2 |
| Colon ca. SW-48 | 8.0 |
| Colon Pool | 9.1 |
| Small Intestine Pool | 8.9 |
| Stomach Pool | 5.1 |
| Bone Marrow Pool | 3.4 |
| Fetal Heart | 1.5 |
| Heart Pool | 3.7 |
| Lymph Node Pool | 8.3 |
| Fetal Skeletal Muscle | 8.1 |
| Skeletal Muscle Pool | 4.3 |
| Spleen Pool | 5.1 |
| Thymus Pool | 7.6 |
| CNS cancer (glio/astro) U87-MG | 6.3 |
| CNS cancer (glio/astro) U-118-MG | 12.7 |
| CNS cancer (neuro; met) SK-N-AS | 6.2 |
| CNS cancer (astro) SF-539 | 7.4 |
| CNS cancer (astro) SNB-75 | 14.1 |
| CNS cancer (glio) SNB-19 | 5.5 |
| CNS cancer (glio) SF-295 | 5.8 |
| Brain (Amygdala) Pool | 24.8 |
| Brain (cerebellum) | 85.9 |
| Brain (fetal) | 16.4 |
| Brain (Hippocampus) Pool | 21.2 |
| Cerebral Cortex Pool | 64.6 |
| Brain (Substantia nigra) Pool | 27.9 |
| Brain (Thalamus) Pool | 51.8 |
| Brain (whole) | 55.5 |
| Spinal Cord Pool | 5.0 |
| Adrenal Gland | 4.9 |
| Pituitary gland Pool | 4.9 |
| Salivary Gland | 2.7 |
| Thyroid (female) | 5.8 |
| Pancreatic ca. CAPAN2 | 9.7 |
| Pancreas Pool | 5.5 |

General_screening_panel_v1.6 Summary: Ag7054 Highest expression of this gene is detected in a ovarian cancer cell line (CT=25.4). Moderate levels of expression of this gene is also seen in cluster of cancer cell lines derived from pancreatic, gastric, colon, lung, liver, renal, breast, ovarian, prostate, squamous cell carcinoma, melanoma and brain cancers. Thus, expression of this gene could be used as a marker to detect the presence of these cancers. Furthermore, therapeutic modulation of the expression or function of this gene may be effective in the treatment of pancreatic, gastric, colon, lung, liver, renal, breast, ovarian, prostate, squamous cell carcinoma, melanoma and brain cancers.

Among tissues with metabolic or endocrine function, this gene is expressed at moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

In addition, this gene is expressed at high levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

Interestingly, this gene is expressed at much higher levels in fetal (CT=28.9) when compared to adult liver (CT=32.7). This observation suggests that expression of this gene can be used to distinguish fetal from adult liver. In addition, the relative overexpression of this gene in fetal tissue suggests that the protein product may enhance liver growth or development in the fetus and thus may also act in a regenerative capacity in the adult. Therefore, therapeutic modulation of the protein encoded by this gene could be useful in treatment of liver related diseases.

E. CG142564-01: Carnitine O-Palmitoyltransferase I.

Expression of gene CG142564-01 was assessed using the primer-probe set Ag6952, described in Table EA. Results of the RTQ-PCR runs are shown in Table EB. Please note that CG142564-02 represents a full-length physical clone.

TABLE EA

Probe Name Ag6952

| | | | Start | SEQ ID |
|---|---|---|---|---|
| Primers | | Length | Position | No |
| Forward | 5′-tctgctaccaatcccagatcc-3′ | 21 | 434 | 264 |
| Probe | TET-5′-tcgacccagagcagcacccca-3′-TAMRA | 21 | 461 | 265 |
| Reverse | 5′-catctgctacagggccaaag-3′ | 20 | 504 | 266 |

TABLE EB
General_screening_panel_v1.6

| | Rel. |
|---|---|
| | Exp. (%) |
| | Ag6952, |
| | Run |
| Tissue Name | 278388893 |
| Adipose | 4.1 |
| Melanoma* Hs688(A).T | 0.8 |
| Melanoma* Hs688(B).T | 1.2 |
| Melanoma* M14 | 21.8 |
| Melanoma* LOXIMVI | 4.6 |
| Melanoma* SK-MEL-5 | 8.5 |
| Squamous cell carcinoma SCC-4 | 1.6 |
| Testis Pool | 31.6 |
| Prostate ca.* (bone met) PC-3 | 9.3 |
| Prostate Pool | 5.8 |
| Placenta | 8.5 |
| Uterus Pool | 0.7 |
| Ovarian ca. OVCAR-3 | 5.0 |
| Ovarian ca. SK-OV-3 | 50.7 |
| Ovarian ca. OVCAR-4 | 1.9 |
| Ovarian ca. OVCAR-5 | 25.3 |
| Ovarian ca. IGROV-1 | 6.9 |
| Ovarian ca. OVCAR-8 | 4.7 |
| Ovary | 3.0 |
| Breast ca. MCF-7 | 9.7 |
| Breast ca. MDA-MB-231 | 9.1 |
| Breast ca. BT 549 | 14.3 |
| Breast ca. T47D | 3.3 |
| Breast ca. MDA-N | 0.8 |
| Breast Pool | 3.1 |
| Trachea | 3.8 |
| Lung | 3.0 |
| Fetal Lung | 7.3 |
| Lung ca. NCI-N417 | 1.2 |
| Lung ca. LX-1 | 22.8 |
| Lung ca. NCI-H146 | 3.6 |
| Lung ca. SHP-77 | 26.4 |
| Lung ca. A549 | 13.4 |
| Lung ca. NCI-H526 | 0.8 |
| Lung ca. NCI-H23 | 13.8 |
| Lung ca. NCI-H460 | 13.9 |
| Lung ca. HOP-62 | 32.8 |
| Lung ca. NCI-H522 | 21.6 |
| Liver | 0.4 |
| Fetal Liver | 2.2 |
| Liver ca. HepG2 | 5.0 |
| Kidney Pool | 2.7 |
| Fetal Kidney | 4.6 |
| Renal ca. 786-0 | 14.6 |
| Renal ca. A498 | 1.8 |
| Renal ca. ACHN | 7.6 |
| Renal ca. UO-31 | 11.9 |
| Renal ca. TK-10 | 20.0 |
| Bladder | 33.4 |
| Gastric ca. (liver met.) NCI-N87 | 81.2 |
| Gastric ca. KATO III | 8.2 |
| Colon ca. SW-948 | 5.4 |
| Colon ca. SW480 | 14.8 |
| Colon ca.* (SW480 met) SW620 | 17.1 |
| Colon ca. HT29 | 1.3 |
| Colon ca. HCT-116 | 14.3 |
| Colon ca. CaCo-2 | 6.7 |
| Colon cancer tissue | 7.6 |
| Colon ca. SW1116 | 4.4 |
| Colon ca. Colo-205 | 4.7 |
| Colon ca. SW-48 | 2.6 |
| Colon Pool | 3.4 |
| Small Intestine Pool | 2.9 |
| Stomach Pool | 2.9 |
| Bone Marrow Pool | 1.5 |
| Fetal Heart | 100.0 |
| Heart Pool | 42.6 |
| Lymph Node Pool | 2.9 |
| Fetal Skeletal Muscle | 17.9 |
| Skeletal Muscle Pool | 21.8 |
| Spleen Pool | 10.4 |
| Thymus Pool | 17.9 |
| CNS cancer (glio/astro) U87-MG | 12.3 |
| CNS cancer (glio/astro) U-118-MG | 25.3 |
| CNS cancer (neuro; met) SK-N-AS | 21.0 |
| CNS cancer (astro) SF-539 | 2.6 |
| CNS cancer (astro) SNB-75 | 16.5 |
| CNS cancer (glio) SNB-19 | 10.1 |
| CNS cancer (glio) SF-295 | 61.1 |
| Brain (Amygdala) Pool | 4.5 |
| Brain (cerebellum) | 39.0 |
| Brain (fetal) | 13.2 |
| Brain (Hippocampus) Pool | 3.6 |
| Cerebral Cortex Pool | 3.4 |
| Brain (Substantia nigra) Pool | 5.3 |
| Brain (Thalamus) Pool | 5.6 |
| Brain (whole) | 3.3 |
| Spinal Cord Pool | 4.8 |
| Adrenal Gland | 6.9 |
| Pituitary gland Pool | 3.2 |
| Salivary Gland | 4.9 |
| Thyroid (female) | 1.1 |
| Pancreatic ca. CAPAN2 | 12.1 |
| Pancreas Pool | 5.0 |

General_screening_panel_v1.6 Summary: Ag6952 Highest expression of this gene is detected in fetal heart (CT=26.7). Moderate to high levels of expression of this gene is also seen in tissues with metabolic/endocrine functions such as pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

Moderate levels of expression of this gene is also seen in cluster of cancer cell lines derived from pancreatic, gastric, colon, lung, liver, renal, breast, ovarian, prostate, squamous cell carcinoma, melanoma and brain cancers. Thus, expression of this gene could be used as a marker to detect the presence of these cancers. Furthermore, therapeutic modulation of the expression or function of this gene may be effective in the treatment of pancreatic, gastric, colon, lung, liver, renal, breast, ovarian, prostate, squamous cell carcinoma, melanoma and brain cancers.

In addition, this gene is expressed at moderate levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

F. CG142797-01: Cathepsin L Like.

Expression of gene CG142797-01 was assessed using the primer-probe set Ag7539, described in Table FA.

TABLE FA

Probe Name Ag7539

| | | | Start | SEQ ID |
|---|---|---|---|---|
| Primers | Sequencs | Length | Position | No |
| Forward | 5′-ctctaacacgtgaccacagtctaga- 3′ | 25 | 68 | 267 |
| Probe | TET-5′-tcttgtgctttgccttccacttggt-3′-TAMRA | 25 | 103 | 268 |
| Reverse | 5′-atcttcatgttctccatgtcatataatc-3′ | 28 | 128 | 269 |

CNS_neurodegeneration_v1.0 Summary: Ag7539 Expression of this gene is low/undetectable (CTs>35) across all of the samples on this panel.

Panel 4.1D Summary: Ag7539 Expression of this gene is low/undetectable (CTs>35) across all of the samples on this panel.

G. CG143216-01: Diacylglycerol Kinase.

Expression of gene CG143216-01 was assessed using the primer-probe sets Ag4554 and Ag7230, described in Tables GA and GB. Results of the RTQ-PCR runs are shown in Tables GC, GD, GE and GF.

TABLE GA

Probe Name Ag4554

| | | | | |
|---|---|---|---|---|
| | | | Start | SEQ ID |
| Primers | Sequence | Length | Position | No |

| | | | | |
|---|---|---|---|---|
| Forward | 5′-aatgctccaggttcaattttct-3′ | 22 | 1349 | 270 |
| Probe | TET-5′-accaaccagcaggaccagtttgactt-3′-TAMRA | 26 | 1390 | 271 |
| Reverse | 5′-gacgcgataaacttcaacaaaa-3′ | 22 | 1419 | 272 |

TABLE GB

Probe Name Ag7230

| | | | Start | SEQ ID |
|---|---|---|---|---|
| Primers | Sequence | Length | Position | No |
| Forward | 5′-gcatatcgttgttggggact-3′ | 20 | 852 | 273 |
| Probe | TET-5′-atggatgtgtcctcagtccaccacaa-3′-TAMRA | 26 | 880 | 274 |
| Reverse | 5′-cacggagtagcgaaggagtg-3′ | 20 | 911 | 275 |

TABLE GC
CNS_neurodegeneration_v1.0

| | Rel. | Rel. |
|---|---|---|
| | Exp. (%) | Exp. (%) |
| | Ag4554, | Ag7230, |
| | Run | Run |
| Tissue Name | 224721290 | 288742189 |
| AD 1 Hippo | 9.3 | 14.1 |
| AD 2 Hippo | 22.2 | 20.2 |
| AD 3 Hippo | 10.6 | 9.7 |
| AD 4 Hippo | 7.1 | 5.3 |
| AD 5 hippo | 100.0 | 100.0 |
| AD 6 Hippo | 36.9 | 42.0 |
| Control 2 Hippo | 22.7 | 23.8 |
| Control 4 Hippo | 7.7 | 10.2 |
| Control (Path) 3 Hippo | 6.9 | 5.2 |
| AD 1 Temporal Ctx | 15.7 | 18.2 |
| AD 2 Temporal Ctx | 20.2 | 20.0 |
| AD 3 Temporal Ctx | 9.9 | 8.0 |
| AD 4 Temporal Ctx | 18.8 | 9.8 |
| AD 5 Inf Temporal Ctx | 97.9 | 81.2 |
| AD 5 Sup Temporal Ctx | 31.6 | 36.3 |
| AD 6 Inf Temporal Ctx | 26.2 | 28.9 |
| AD 6 Sup Temporal Ctx | 29.1 | 33.7 |
| Control 1 Temporal Ctx | 9.5 | 5.1 |
| Control 2 Temporal Ctx | 39.0 | 43.2 |
| Control 3 Temporal Ctx | 10.1 | 11.4 |
| Control 4 Temporal Ctx | 6.6 | 6.7 |
| Control (Path) 1 Temporal Ctx | 32.8 | 35.1 |
| Control (Path) 2 Temporal Ctx | 20.4 | 22.8 |
| Control (Path) 3 Temporal Ctx | 5.7 | 5.3 |
| Control (Path) 4 Temporal Ctx | 20.0 | 19.2 |
| AD 1 Occipital Ctx | 7.3 | 18.6 |
| AD 2 Occipital Ctx (Missing) | 0.0 | 0.0 |
| AD 3 Occipital Ctx | 11.3 | 8.0 |
| AD 4 Occipital Ctx | 19.8 | 13.4 |
| AD 5 Occipital Ctx | 15.9 | 18.0 |
| AD 6 Occipital Ctx | 53.2 | 54.3 |
| Control 1 Occipital Ctx | 4.5 | 3.9 |
| Control 2 Occipital Ctx | 81.8 | 90.8 |
| Control 3 Occipital Ctx | 14.4 | 14.7 |
| Control 4 Occipital Ctx | 6.4 | 6.8 |
| Control (Path) 1 Occipital Ctx | 45.4 | 57.8 |
| Control (Path) 2 Occipital Ctx | 6.1 | 6.1 |
| Control (Path) 3 Occipital Ctx | 5.1 | 5.2 |
| Control (Path) 4 Occipital Ctx | 12.6 | 12.8 |
| Control 1 Parietal Ctx | 6.4 | 5.7 |
| Control 2 Parietal Ctx | 26.4 | 26.4 |
| Control 3 Parietal Ctx | 18.0 | 19.6 |
| Control (Path) 1 Parietal Ctx | 56.3 | 70.7 |
| Control (Path) 2 Parietal Ctx | 15.7 | 15.2 |
| Control (Path) 3 Parietal Ctx | 5.5 | 5.1 |
| Control (Path) 4 Parietal Ctx | 41.5 | 36.3 |

TABLE GD
General_screening_panel_v1.4

| | Rel. |
|---|---|
| | Exp. (%) |
| | Ag4554, |
| | Run |
| Tissue Name | 222809973 |
| Adipose | 5.4 |
| Melanoma* Hs688(A).T | 45.1 |
| Melanoma* Hs688(B).T | 45.1 |
| Melanoma* M14 | 85.9 |
| Melanoma* LOXIMVI | 21.9 |
| Melanoma* SK-MEL-5 | 69.7 |
| Squamous cell carcinoma SCC-4 | 26.8 |
| Testis Pool | 6.8 |
| Prostate ca.* (bone met) PC-3 | 29.9 |
| Prostate Pool | 6.9 |
| Placenta | 5.7 |
| Uterus Pool | 4.8 |
| Ovarian ca. OVCAR-3 | 14.9 |
| Ovarian ca. SK-OV-3 | 100.0 |
| Ovarian ca. OVCAR-4 | 10.2 |
| Ovarian ca. OVCAR-5 | 36.1 |
| Ovarian ca. IGROV-1 | 20.3 |
| Ovarian ca. OVCAR-8 | 16.0 |
| Ovary | 15.0 |
| Breast ca. MCF-7 | 16.5 |
| Breast ca. MDA-MB-231 | 51.1 |
| Breast ca. BT 549 | 47.3 |
| Breast ca. T47D | 62.0 |
| Breast ca. MDA-N | 17.8 |
| Breast Pool | 12.5 |
| Trachea | 12.3 |
| Lung | 1.2 |
| Fetal Lung | 27.4 |
| Lung ca. NCI-N417 | 8.0 |
| Lung ca. LX-1 | 52.1 |
| Lung ca. NCI-H146 | 22.5 |
| Lung ca. SHP-77 | 97.9 |
| Lung ca. A549 | 25.0 |
| Lung ca. NCI-H526 | 0.0 |
| Lung ca. NCI-H23 | 45.1 |
| Lung ca. NCI-H460 | 15.9 |
| Lung ca. HOP-62 | 27.4 |
| Lung ca. NCI-H522 | 27.9 |
| Liver | 3.7 |
| Fetal Liver | 12.0 |
| Liver ca. HepG2 | 28.1 |
| Kidney Pool | 25.0 |
| Fetal Kidney | 13.7 |
| Renal ca. 786-0 | 24.0 |
| Renal ca. A498 | 4.5 |
| Renal ca. ACHN | 6.3 |
| Renal ca. UO-31 | 18.8 |
| Renal ca. TK-10 | 34.6 |
| Bladder | 15.8 |
| Gastric ca. (liver met.) NCI-N87 | 21.3 |
| Gastric ca. KATO III | 84.1 |
| Colon ca. SW-948 | 0.7 |
| Colon ca. SW480 | 52.5 |
| Colon ca.* (SW480 met) SW620 | 27.0 |
| Colon ca. HT29 | 12.5 |
| Colon ca. HCT-116 | 72.7 |
| Colon ca. CaCo-2 | 25.5 |
| Colon cancer tissue | 24.1 |
| Colon ca. SW1116 | 8.5 |
| Colon ca. Colo-205 | 12.9 |
| Colon ca. SW-48 | 6.5 |
| Colon Pool | 15.0 |
| Small Intestine Pool | 17.8 |
| Stomach Pool | 9.0 |
| Bone Marrow Pool | 5.0 |
| Fetal Heart | 23.5 |
| Heart Pool | 12.2 |
| Lymph Node Pool | 15.1 |
| Fetal Skeletal Muscle | 4.6 |
| Skeletal Muscle Pool | 12.0 |
| Spleen Pool | 10.7 |
| Thymus Pool | 26.2 |
| CNS cancer (glio/astro) U87-MG | 65.1 |
| CNS cancer (glio/astro) U-118-MG | 79.0 |
| CNS cancer (neuro; met) SK-N-AS | 48.6 |
| CNS cancer (astro) SF-539 | 23.3 |
| CNS cancer (astro) SNB-75 | 89.5 |
| CNS cancer (glio) SNB-19 | 21.8 |
| CNS cancer (glio) SF-295 | 63.7 |
| Brain (Amygdala) Pool | 14.8 |
| Brain (cerebellum) | 90.8 |
| Brain (fetal) | 30.4 |
| Brain (Hippocampus) Pool | 15.0 |
| Cerebral Cortex Pool | 29.3 |
| Brain (Substantia nigra) Pool | 31.2 |
| Brain (Thalamus) Pool | 27.7 |
| Brain (whole) | 29.3 |
| Spinal Cord Pool | 11.8 |
| Adrenal Gland | 29.1 |
| Pituitary gland Pool | 24.8 |
| Salivary Gland | 11.6 |
| Thyroid (female) | 11.5 |
| Pancreatic ca. CAPAN2 | 10.4 |
| Pancreas Pool | 21.8 |

TABLE GE
Panel 4.1D

| | Rel. | Rel. |
|---|---|---|
| | Exp. (%) | Exp. (%) |
| | Ag4554, | Ag7230, |
| | Run | Run |
| Tissue Name | 199319739 | 288211134 |
| Secondary Th1 act | 70.2 | 48.3 |
| Secondary Th2 act | 44.8 | 30.4 |
| Secondary Tr1 act | 64.2 | 17.8 |
| Secondary Th1 rest | 17.7 | 6.7 |
| Secondary Th2 rest | 22.4 | 6.6 |
| Secondary Tr1 rest | 17.0 | 6.0 |
| Primary Th1 act | 27.7 | 6.0 |
| Primary Th2 act | 42.3 | 24.8 |
| Primary Tr1 act | 39.5 | 31.4 |
| Primary Th1 rest | 17.2 | 12.2 |
| Primary Th2 rest | 11.0 | 10.1 |
| Primary Tr1 rest | 39.2 | 1.2 |
| CD45RA CD4 lymphocyte act | 39.8 | 18.7 |
| CD45RO CD4 lymphocyte act | 44.4 | 31.4 |
| CD8 lymphocyte act | 41.2 | 10.8 |
| Secondary CD8 lymphocyte rest | 43.5 | 9.9 |
| Secondary CD8 lymphocyte act | 11.2 | 4.4 |
| CD4 lymphocyte none | 19.2 | 5.0 |
| 2ry Th1/Th2/Tr1_anti-CD95 CH11 | 40.9 | 11.2 |
| LAK cells rest | 21.0 | 8.0 |
| LAK cells IL-2 | 23.0 | 13.0 |
| LAK cells IL-2 + IL-12 | 12.7 | 1.5 |
| LAK cells IL-2 + IFN gamma | 14.6 | 5.6 |
| LAK cells IL-2 + IL-18 | 18.7 | 7.7 |
| LAK cells PMA/ionomycin | 23.8 | 14.3 |
| NK Cells IL-2 rest | 42.9 | 35.8 |
| Two Way MLR 3 day | 22.5 | 9.9 |
| Two Way MLR 5 day | 20.9 | 3.3 |
| Two Way MLR 7 day | 21.2 | 10.2 |
| PBMC rest | 12.0 | 6.8 |
| PBMC PWM | 19.3 | 5.1 |
| PBMC PHA-L | 29.9 | 14.4 |
| Ramos (B cell) none | 19.3 | 6.5 |
| Ramos (B cell) ionomycin | 21.3 | 13.7 |
| B lymphocytes PWM | 18.2 | 9.9 |
| B lymphocytes CD40L and IL-4 | 26.4 | 25.7 |
| EOL-1 dbcAMP | 29.3 | 26.2 |
| EOL-1 dbcAMP PMA/ionomycin | 23.0 | 7.5 |
| Dendritic cells none | 28.9 | 17.6 |
| Dendritic cells LPS | 9.0 | 2.8 |
| Dendritic cells anti-CD40 | 40.6 | 8.3 |
| Monocytes rest | 20.7 | 7.6 |
| Monocytes LPS | 18.2 | 15.7 |
| Macrophages rest | 20.0 | 8.2 |
| Macrophages LPS | 4.0 | 2.0 |
| HUVEC none | 57.8 | 31.9 |
| HUVEC starved | 64.2 | 50.0 |
| HUVEC IL-1beta | 62.9 | 38.4 |
| HUVEC IFN gamma | 50.3 | 35.1 |
| HUVEC TNF alpha + IFN gamma | 18.2 | 14.0 |
| HUVEC TNF alpha + IL4 | 43.2 | 13.1 |
| HUVEC IL-11 | 38.2 | 16.7 |
| Lung Microvascular EC none | 100.0 | 100.0 |
| Lung Microvascular EC TNFalpha + | 82.4 | 42.0 |
| IL-1beta | | |
| Microvascular Dermal EC none | 40.3 | 9.7 |
| Microsvasular Dermal EC | 28.3 | 7.1 |
| TNFalpha + IL-1beta | | |
| Bronchial epithelium TNFalpha + | 17.7 | 5.6 |
| IL1beta | | |
| Small airway epithelium none | 4.5 | 3.6 |
| Small airway epithelium | 11.4 | 6.6 |
| TNFalpha + IL-1beta | | |
| Coronery artery SMC rest | 24.8 | 14.1 |
| Coronery artery SMC TNFalpha + | 24.7 | 19.8 |
| IL-1beta | | |
| Astrocytes rest | 11.7 | 10.2 |
| Astrocytes TNFalpha + IL-1beta | 7.8 | 3.8 |
| KU-812 (Basophil) rest | 5.8 | 4.3 |
| KU-812 (Basophil) PMA/ionomycin | 7.9 | 5.7 |
| CCD1106 (Keratinocytes) none | 14.6 | 13.4 |
| CCD1106 (Keratinocytes) | 5.7 | 2.1 |
| TNFalpha + IL-1beta | | |
| Liver cirrhosis | 3.0 | 4.0 |
| NCI-H292 none | 3.4 | 7.5 |
| NCI-H292 IL-4 | 7.1 | 8.0 |
| NCI-H292 IL-9 | 9.7 | 6.6 |
| NCI-H292 IL-13 | 10.7 | 6.3 |
| NCI-H292 IFN gamma | 3.2 | 1.5 |
| HPAEC none | 31.0 | 13.9 |
| HPAEC TNF alpha + IL-1 beta | 52.5 | 31.9 |
| Lung fibroblast none | 16.0 | 7.7 |
| Lung fibroblast TNF alpha + | 16.8 | 9.6 |
| IL-1 beta | | |
| Lung fibroblast IL-4 | 16.3 | 7.6 |
| Lung fibroblast IL-9 | 23.2 | 11.4 |
| Lung fibroblast IL-13 | 13.8 | 7.0 |
| Lung fibroblast IFN gamma | 7.1 | 6.1 |
| Dermal fibroblast CCD1070 rest | 22.7 | 36.6 |
| Dermal fibroblast CCD1070 TNF | 63.7 | 59.5 |
| alpha | | |
| Dermal fibroblast CCD1070 IL-1 | 29.9 | 19.3 |
| beta | | |
| Dermal fibroblast IFN gamma | 7.0 | 5.6 |
| Dermal fibroblast IL-4 | 20.6 | 12.9 |
| Dermal Fibroblasts rest | 15.2 | 20.7 |
| Neutrophils TNFa + LPS | 18.4 | 16.0 |
| Neutrophils rest | 16.3 | 20.6 |
| Colon | 14.1 | 3.9 |
| Lung | 9.9 | 2.6 |
| Thymus | 39.2 | 7.4 |
| Kidney | 18.8 | 11.6 |

TABLE GF
Panel 5 Islet

| | Rel. |
|---|---|
| | Exp. (%) |
| | Ag4554, |
| | Run |
| Tissue Name | 306350410 |
| 97457_Patient-02go_adipose | 5.0 |
| 97476_Patient-07sk_skeletal muscle | 0.0 |
| 97477_Patient-07ut_uterus | 5.4 |
| 97478_Patient-07pl_placenta | 2.6 |
| 99167_Bayer Patient 1 | 100.0 |
| 97482_Patient-08ut_uterus | 2.4 |
| 97483_Patient-08pl_placenta | 1.9 |
| 97486_Patient-09sk_skeletal muscle | 3.4 |
| 97487_Patient-09ut_uterus | 3.4 |
| 97488_Patient-09pl_placenta | 0.9 |
| 97492_Patient-10ut_uterus | 5.6 |
| 97493_Patient-10pl_placenta | 6.0 |
| 97495_Patient-11go_adipose | 4.7 |
| 97496_Patient-11sk_skeletal muscle | 3.4 |
| 97497_Patient-11ut_uterus | 6.0 |
| 97498_Patient-11pl_placenta | 2.0 |
| 97500_Patient-12go_adipose | 8.7 |
| 97501_Patient-12sk_skeletal muscle | 14.2 |
| 97502_Patient-12ut_uterus | 12.3 |
| 97503_Patient-12pl_placenta | 3.5 |
| 94721_Donor 2 U - A_Mesenchymal Stem Cells | 21.6 |
| 94722_Donor 2 U - B_Mesenchymal Stem Cells | 6.3 |
| 94723_Donor 2 U - C_Mesenchymal Stem Cells | 20.2 |
| 94709_Donor 2 AM - A_adipose | 20.3 |
| 94710_Donor 2 AM - B_adipose | 12.8 |
| 94711_Donor 2 AM - C_adipose | 9.5 |
| 94712_Donor 2 AD - A_adipose | 18.0 |
| 94713_Donor 2 AD - B_adipose | 34.4 |
| 94714_Donor 2 AD - C_adipose | 17.3 |
| 94742_Donor 3 U - A_Mesenchymal Stem Cells | 10.0 |
| 94743_Donor 3 U - B_Mesenchymal Stem Cells | 9.7 |
| 94730_Donor 3 AM - A_adipose | 29.1 |
| 94731_Donor 3 AM - B_adipose | 47.0 |
| 94732_Donor 3 AM - C_adipose | 33.9 |
| 94733_Donor 3 AD - A_adipose | 46.3 |
| 94734_Donor 3 AD - B_adipose | 72.7 |
| 94735_Donor 3 AD - C_adipose | 13.7 |
| 77138_Liver_HepG2untreated | 41.5 |
| 73556_Heart_Cardiac stromal cells (primary) | 8.5 |
| 81735_Small Intestine | 18.0 |
| 72409_Kidney_Proximal Convoluted Tubule | 9.3 |
| 82685_Small intestine_Duodenum | 20.2 |
| 90650_Adrenal_Adrenocortical adenoma | 10.1 |
| 72410_Kidney_HRCE | 16.8 |
| 72411_Kidney_HRE | 6.8 |
| 73139_Uterus_Uterine smooth muscle cells | 19.5 |

CNS_neurodegeneration_v1.0 Summary: Ag4554/Ag7230 Two experiments with different probe-primer sets are in excellent agreement. This panel confirms the expression of this gene at low levels in the brains of an independent group of individuals. However, no differential expression of this gene was detected between Alzheimer's diseased postmortem brains and those of non-demented controls in this experiment. Please see Panel 1.4 for a discussion of this gene in treatment of central nervous system disorders.

General_screeningpanel_v1.4 Summary: Ag4554 Highest expression of this gene is detected in a ovarian cancer cell line (CT=25.4). Moderate levels of expression of this gene is also seen in cluster of cancer cell lines derived from pancreatic, gastric, colon, lung, liver, renal, breast, ovarian, prostate, squamous cell carcinoma, melanoma and brain cancers. Thus, expression of this gene could be used as a marker to detect the presence of these cancers. Furthermore, therapeutic modulation of the expression or function of this gene may be effective in the treatment of pancreatic, gastric, colon, lung, liver, renal, breast, ovarian, prostate, squamous cell carcinoma, melanoma and brain cancers.

Among tissues with metabolic or endocrine function, this gene is expressed at moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

In addition, this gene is expressed at high levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

Interestingly, this gene is expressed at much higher levels in fetal (CT=27.3) when compared to adult lung (CT=31.8). This observation suggests that expression of this gene can be used to distinguish fetal from adult lung. In addition, the relative overexpression of this gene in fetal tissue suggests that the protein product may enhance lung growth or development in the fetus and thus may also act in a regenerative capacity in the adult. Therefore, therapeutic modulation of the protein encoded by this gene could be useful in treatment of lung related diseases.

Panel 4.1D Summary: Ag4554/Ag7230 Two experiments with different probe-primer sets are in excellent agreement. Highest expression of this gene is detected in lung microvascular endothelial cells (CTs=28-29). This gene is expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screening_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Panel 5 Islet Summary: Ag4554 Highest expression of this gene is detected in islet cells (CT=29.8). This gene shows a widespread expression pattern which correlates with the pattern seen in panel 1.4. Please see panel 1.4 for further discussion of this gene.

H. CG143787-01: Disintegrin Protease.

Expression of gene CG143787-01 was assessed using the primer-probe sets Ag6532, Ag6655 and Ag7048, described in Tables HA, HB and HC. Please note that CG143787-01 represents a full-length physical clone.

TABLE HA

Probe Name Ag6532

| | | | Start | SEQ ID |
|---|---|---|---|---|
| Primers | Sequencs | Length | Position | No |
| Forward | 5′-atcatcaccaaagataccttttatctc-3′ | 27 | 474 | 276 |
| Probe | TET-5′-agaaaccaaagtgcctgctgcaagc-3′TAMRA | 25 | 501 | 277 |
| Reverse | 5′-gtgttgtcattatatttgtaggaataggt-3′ | 29 | 526 | 278 |

TABLE HB

Probe Name Ag6655

| | | | | |
|---|---|---|---|---|
| | | | Start | SEQ ID |
| Primers | Sequenes | Length | Position | No |

| | | | | |
|---|---|---|---|---|
| Forward | 5′-atcatcaccaaagataccttttatctc-3′ | 27 | 474 | 279 |
| Probe | TET-5′-agaaaccaaagtgcctgctgcaagc-3′TAMRA | 25 | 501 | 280 |
| Reverse | 5′-gtgttgtcattatatttgtaggaataggt-3′ | 29 | 526 | 281 |

TABLE HC

Probe Name Ag7048

| | | | | |
|---|---|---|---|---|
| | | | Start | SEQ ID |
| Primers | Sequence | Length | Position | No |
| Forward | 5′-acatcatcaccaaagatacctttta-3′ | 25 | 472 | 282 |
| Probe | TET-5′-caaagtgcctgctgcaagcacctatt-3′-TAMRA | 26 | 507 | 283 |
| Reverse | 5′-gttcccacacactggtgttg-3′ | 20 | 549 | 284 |

General_screening_panel_v1.6 Summary: Ag6655/Ag7048 Expression of this gene is low/undetectable (CTs>35) across all of the samples on this panel.

Panel 4.1D Summary: Ag6655 Expression of this gene is low/undetectable (CTs>35) across all of the samples on this panel.

I. CG144112-01: Neuropsin Precursor.

Expression of gene CG144112-01 was assessed using the primer-probe set Ag7123, described in Table IA. Please note that CG56663-01 represents a full-length physical clone.

TABLE IA

Probe Name Ag7123

| | | | Start | SEQ ID |
|---|---|---|---|---|
| Primers | Sequencs | Length | Position | No |
| Forward | 5′-gcctgggcaggaaatacac-3′ | 19 | 353 | 285 |
| Probe | TET-5′-tacgcctgggagaccacagcctacag-3′TAMRA | 26 | 325 | 286 |
| Reverse | 5′-tctcggggactgcacttct-3′ | 19 | 292 | 287 |

CNS_neurodegeneration_v1.0 Summary: Ag7123 Expression of this gene is low/undetectable (CTs>35) across all of the samples on this panel.

Panel 4.1D Summary: Ag7123 Expression of this gene is low/undetectable (CTs>35) across all of the samples on this panel.

J. CG144112-04: Kallikrein-8.

Expression of gene CG144112-04 was assessed using the primer-probe set Ag5271, described in Table JA.

TABLE JA

Probe Name Ag5271

| | | | Start | SEQ ID |
|---|---|---|---|---|
| Primers | Sequence | Length | Position | No |
| Forward | 5′-gcagggcagggcgattct-3′ | 18 | 97 | 288 |
| Probe | TET-5′-cacatcctggggctcagacccctgtg-3′-TAMRA | 26 | 153 | 289 |
| Reverse | 5′-ctagaatcagcccttgctgccta-3′ | 23 | 245 | 290 |

CNS_neurodegeneration_v1.0 Summary: Ag5271 Expression of this gene is low/undetectable (CTs>35) across all of the samples on this panel.

Panel 4.1D Summary: Ag5271 Expression of this gene is low/undetectable (CTs>35) across all of the samples on this panel.

K. CG144686-01: Mast Cell Carboxypeptidase A Precursor.

Expression of gene CG144686-01 was assessed using the primer-probe set Ag6864, described in Table KA. Results of the RTQ-PCR runs are shown in Tables KB and KC. Please note that CG144686-01 represents a full-length physical clone.

TABLE KA

Probe Name Ag6864

| | | | Start | SEQ ID |
|---|---|---|---|---|
| Primers | Sequencs | Length | Position | No |
| Forward | 5′-aaccagtgagctccgaga-3′ | 18 | 122 | 291 |
| Probe | TET-5′-caaatttggttttctccttccagaatcc-3′-TAMRA | 28 | 146 | 292 |
| Reverse | 5′-tctgcacgttggctttat-3′ | 18 | 177 | 293 |

TABLE KB
General_screening_panel_v1.6

| | Rel. |
|---|---|
| | Exp. (%) |
| | Ag6864, |
| | Run |
| Tissue Name | 278387547 |
| Adipose | 15.0 |
| Melanoma* Hs688(A).T | 0.3 |
| Melanoma* Hs688(B).T | 0.7 |
| Melanoma* M14 | 0.0 |
| Melanoma* LOXIMVI | 0.0 |
| Melanoma* SK-MEL-5 | 0.0 |
| Squamous cell carcinoma SCC-4 | 0.0 |
| Testis Pool | 7.6 |
| Prostate ca.* (bone met) PC-3 | 0.0 |
| Prostate Pool | 16.4 |
| Placenta | 0.1 |
| Uterus Pool | 15.8 |
| Ovarian ca. OVCAR-3 | 0.0 |
| Ovarian ca. SK-OV-3 | 0.0 |
| Ovarian ca. OVCAR-4 | 0.0 |
| Ovarian ca. OVCAR-5 | 0.0 |
| Ovarian ca. IGROV-1 | 0.0 |
| Ovarian ca. OVCAR-8 | 0.0 |
| Ovary | 2.5 |
| Breast ca. MCF-7 | 0.0 |
| Breast ca. MDA-MB-231 | 0.0 |
| Breast ca. BT 549 | 0.7 |
| Breast ca. T47D | 0.0 |
| Breast ca. MDA-N | 0.0 |
| Breast Pool | 0.0 |
| Trachea | 2.5 |
| Lung | 2.7 |
| Fetal Lung | 5.3 |
| Lung ca. NCI-N417 | 0.0 |
| Lung ca. LX-1 | 0.0 |
| Lung ca. NCI-H146 | 0.0 |
| Lung ca. SHP-77 | 4.5 |
| Lung ca. A549 | 0.0 |
| Lung ca. NCI-H526 | 0.0 |
| Lung ca. NCI-H23 | 0.0 |
| Lung ca. NCI-H460 | 0.0 |
| Lung ca. HOP-62 | 0.9 |
| Lung ca. NCI-H522 | 0.0 |
| Liver | 0.0 |
| Fetal Liver | 6.0 |
| Liver ca. HepG2 | 0.0 |
| Kidney Pool | 51.4 |
| Fetal Kidney | 1.1 |
| Renal ca. 786-0 | 0.2 |
| Renal ca. A498 | 0.0 |
| Renal ca. ACHN | 0.0 |
| Renal ca. UO-31 | 0.2 |
| Renal ca. TK-10 | 0.0 |
| Bladder | 0.0 |
| Gastric ca. (liver met.) NCI-N87 | 0.0 |
| Gastric ca. KATO III | 0.0 |
| Colon ca. SW-948 | 0.0 |
| Colon ca. SW480 | 0.0 |
| Colon ca.* (SW480 met) SW620 | 0.0 |
| Colon ca. HT29 | 0.0 |
| Colon ca. HCT-116 | 0.0 |
| Colon ca. CaCo-2 | 0.0 |
| Colon cancer tissue | 70.7 |
| Colon ca. SW1116 | 0.0 |
| Colon ca. Colo-205 | 0.0 |
| Colon ca. SW-48 | 0.0 |
| Colon Pool | 78.5 |
| Small Intestine Pool | 0.0 |
| Stomach Pool | 20.0 |
| Bone Marrow Pool | 23.2 |
| Fetal Heart | 4.6 |
| Heart Pool | 20.0 |
| Lymph Node Pool | 100.0 |
| Fetal Skeletal Muscle | 5.5 |
| Skeletal Muscle Pool | 1.5 |
| Spleen Pool | 3.0 |
| Thymus Pool | 18.2 |
| CNS cancer (glio/astro) U87-MG | 0.0 |
| CNS cancer (glio/astro) U-118-MG | 1.8 |
| CNS cancer (neuro; met) SK-N-AS | 0.0 |
| CNS cancer (astro) SF-539 | 0.0 |
| CNS cancer (astro) SNB-75 | 0.0 |
| CNS cancer (glio) SNB-19 | 0.0 |
| CNS cancer (glio) SF-295 | 0.0 |
| Brain (Amygdala) Pool | 0.0 |
| Brain (cerebellum) | 0.0 |
| Brain (fetal) | 0.0 |
| Brain (Hippocampus) Pool | 0.0 |
| Cerebral Cortex Pool | 0.0 |
| Brain (Substantia nigra) Pool | 0.0 |
| Brain (Thalamus) Pool | 0.0 |
| Brain (whole) | 0.0 |
| Spinal Cord Pool | 0.0 |
| Adrenal Gland | 0.7 |
| Pituitary gland Pool | 1.0 |
| Salivary Gland | 0.0 |
| Thyroid (female) | 0.2 |
| Pancreatic ca. CAPAN2 | 0.0 |
| Pancreas Pool | 10.4 |

TABLE KC
Panel 5 Islet

| | Rel. | Rel. |
|---|---|---|
| | Exp. (%) | Exp. (%) |
| | Ag6864 | Ag6864, |
| | Run | Run |
| Tissue Name | 305424858 | 307650498 |
| 97457_Patient-02go_adipose | 5.5 | 34.9 |
| 97476_Patient-07sk_skeletal muscle | 0.0 | 0.0 |
| 97477_Patient-07ut_uterus | 1.4 | 32.1 |
| 97478_Patient-07pl_placenta | 0.0 | 4.7 |
| 99167_Bayer Patient 1 | 0.0 | 0.0 |
| 97482_Patient-08ut_uterus | 0.0 | 0.0 |
| 97483_Patient-08pl_placenta | 0.0 | 0.0 |
| 97486_Patient-09sk_skeletal muscle | 7.6 | 15.5 |
| 97487_Patient-09ut_uterus | 28.7 | 11.2 |
| 97488_Patient-09pl_placenta | 1.4 | 0.0 |
| 97492_Patient-10ut_uterus | 10.4 | 7.2 |
| 97493_Patient-10pl_placenta | 0.0 | 5.9 |
| 97495_Patient-11go_adipose | 20.0 | 5.0 |
| 97496_Patient-11sk_skeletal muscle | 6.0 | 8.7 |
| 97497_Patient-11ut_uterus | 45.1 | 65.1 |
| 97498_Patient-11pl_placenta | 0.0 | 0.0 |
| 97500_Patient-12go_adipose | 59.9 | 59.9 |
| 97501_Patient-12sk_skeletal muscle | 100.0 | 100.0 |
| 97502_Patient-12ut_uterus | 29.1 | 97.3 |
| 97503_Patient-12pl_placenta | 5.0 | 2.3 |
| 94721_Donor 2 U - A_Mesenchymal Stem | 0.0 | 0.0 |
| Cells | | |
| 94722_Donor 2 U - B_Mesenchymal Stem | 0.0 | 0.0 |
| Cells | | |
| 94723_Donor 2 U - C_Mesenchymal Stem | 1.5 | 0.0 |
| Cells | | |
| 94709_Donor 2 AM - A_adipose | 0.0 | 0.0 |
| 94710_Donor 2 AM - B_adipose | 0.0 | 0.0 |
| 94711_Donor 2 AM - C_adipose | 0.0 | 0.0 |
| 94712_Donor 2 AD - A_adipose | 0.0 | 0.0 |
| 94713_Donor 2 AD - B_adipose | 0.0 | 0.0 |
| 94714_Donor 2 AD - C_adipose | 2.3 | 0.0 |
| 94742_Donor 3 U - A_Mesenchymal Stem | 0.0 | 0.0 |
| Cells | | |
| 94743_Donor 3 U - B_Mesenchymal Stem | 0.0 | 0.0 |
| Cells | | |
| 94730_Donor 3 AM - A_adipose | 0.0 | 0.0 |
| 94731_Donor 3 AM - B_adipose | 0.0 | 1.9 |
| 94732_Donor 3 AM - C_adipose | 0.0 | 0.0 |
| 94733_Donor 3 AD - A_adipose | 0.0 | 0.0 |
| 94734_Donor 3 AD - B_adipose | 0.0 | 0.0 |
| 94735_Donor 3 AD - C_adipose | 0.0 | 0.0 |
| 77138_Liver_HepG2untreated | 0.0 | 0.0 |
| 73556_Heart_Cardiac stromal cells | 5.1 | 3.2 |
| (primary) | | |
| 81735_Small Intestine | 73.2 | 65.1 |
| 72409_Kidney_Proximal Convoluted | 0.0 | 0.0 |
| Tubule | | |
| 82685_Small intestine_Duodenum | 59.0 | 67.4 |
| 90650_Adrenal_Adrenocortical adenoma | 0.0 | 0.0 |
| 72410_Kidney_HRCE | 0.0 | 0.0 |
| 72411_Kidney_HRE | 0.0 | 0.0 |
| 73139_Uterus_Uterine smooth muscle | 0.0 | 0.0 |
| cells | | |

General_screening_panel_v1.6 Summary: Ag6864 Highest expression of this gene is seen in lymph node (CT=29). Moderate levels of expression are also seen predominantly in normal tissue, including adipose, colon, heart, thymus, prostate, and kidney, as well as in colon cancer tissue. Thus, expression of this gene could be used to identify these samples and tissues. Modulation of the expression of this gene may also be effective in the treatment of diseases of these tissues, including cancer, obesity and diabetes.

Panel 5 Islet Summary: Ag6864 Two experiments with the same probe and primer produce results that are in excellent agreement. Highest expression of this gene is seen in skeletal muscle (CTs=33.5). Please see Panel 1.6 for discussion of this gene.

L. CG144906-01: Testisin Precursor.

Expression of gene CG144906-01 was assessed using the primer-probe set Ag6915, described in Table LA. Please note that CG144906-01 represents a full-length physical clone.

TABLE LA

Probe Name AG6915

| | | | Start | SEQ ID |
|---|---|---|---|---|
| Primers | Sequence | Length | Position | No |
| Forward | 5′-catgccatcctccacattt-3′ | 19 | 337 | 294 |
| Probe | TET-5′-cagcagtctgtccggttctcaaactc-3′-TAMRA | 26 | 356 | 295 |
| Reverse | 5′-gtgcctcatcctctttgatgta-3′ | 22 | 398 | 296 |

General_screening_panel_v1.6 Summary: Ag6915 Expression of this gene is low/undetectable (CTs>35) across all of the samples on this panel.

M. CG144997-01: RNase H I.

Expression of gene CG144997-01 was assessed using the primer-probe set Ag7057, described in Table MA. Results of the RTQ-PCR runs are shown in Table MB. Please note that CG144997-01 represents a full-length physical clone.

TABLE MA

Probe Name Ag7057

| | | | Start | SEQ ID |
|---|---|---|---|---|
| Primers | | Length | Position | No |
| Forward | 5′-gtaaacgccgattcctgct-3′ | 19 | 468 | 297 |
| Probe | TET-5′-cttctacgcccattactggagcagca-3′-TAMRA | 26 | 493 | 298 |
| Reverse | 5′-gaatgagtgcagagacacgttt-3′ | 22 | 558 | 299 |

TABLE MB
General_screening_panel_v1.6

| | Rel. |
|---|---|
| | Exp. (%) |
| | Ag7057, |
| | Run |
| Tissue Name | 282273884 |
| Adipose | 3.9 |
| Melanoma* Hs688(A).T | 23.8 |
| Melanoma* Hs688(B).T | 28.3 |
| Melanoma* M14 | 50.7 |
| Melanoma* LOXIMVI | 57.8 |
| Melanoma* SK-MEL-5 | 51.4 |
| Squamous cell carcinoma SCC-4 | 22.5 |
| Testis Pool | 9.0 |
| Prostate ca.* (bone met) PC-3 | 60.3 |
| Prostate Pool | 5.4 |
| Placenta | 4.5 |
| Uterus Pool | 1.9 |
| Ovarian ca. OVCAR-3 | 31.2 |
| Ovarian ca. SK-OV-3 | 31.4 |
| Ovarian ca. OVCAR-4 | 17.1 |
| Ovarian ca. OVCAR-5 | 39.0 |
| Ovarian ca. IGROV-1 | 13.3 |
| Ovarian ca. OVCAR-8 | 15.0 |
| Ovary | 4.9 |
| Breast ca. MCF-7 | 21.8 |
| Breast ca. MDA-MB-231 | 17.3 |
| Breast ca. BT 549 | 24.8 |
| Breast ca. T47D | 9.5 |
| Breast ca. MDA-N | 22.7 |
| Breast Pool | 12.3 |
| Trachea | 7.3 |
| Lung | 1.9 |
| Fetal Lung | 8.6 |
| Lung ca. NCI-N417 | 10.1 |
| Lung ca. LX-1 | 22.4 |
| Lung ca. NCI-H146 | 11.9 |
| Lung ca. SHP-77 | 82.9 |
| Lung ca. A549 | 54.0 |
| Lung ca. NCI-H526 | 8.9 |
| Lung ca. NCI-H23 | 37.9 |
| Lung ca. NCI-H460 | 37.1 |
| Lung ca. HOP-62 | 12.1 |
| Lung ca. NCI-H522 | 56.6 |
| Liver | 0.8 |
| Fetal Liver | 6.7 |
| Liver ca. HepG2 | 18.6 |
| Kidney Pool | 10.8 |
| Fetal Kidney | 5.8 |
| Renal ca. 786-0 | 21.6 |
| Renal ca. A498 | 17.1 |
| Renal ca. ACHN | 17.6 |
| Renal ca. UO-31 | 18.0 |
| Renal ca. TK-10 | 33.9 |
| Bladder | 15.7 |
| Gastric ca. (liver met.) NCI-N87 | 49.0 |
| Gastric ca. KATO III | 100.0 |
| Colon ca. SW-948 | 11.4 |
| Colon ca. SW480 | 76.3 |
| Colon ca.* (SW480 met) SW620 | 34.9 |
| Colon ca. HT29 | 15.8 |
| Colon ca. HCT-116 | 36.6 |
| Colon ca. CaCo-2 | 42.0 |
| Colon cancer tissue | 17.6 |
| Colon ca. SW1116 | 5.4 |
| Colon ca. Colo-205 | 10.4 |
| Colon ca. SW-48 | 6.8 |
| Colon Pool | 9.5 |
| Small Intestine Pool | 5.7 |
| Stomach Pool | 5.1 |
| Bone Marrow Pool | 3.3 |
| Fetal Heart | 4.7 |
| Heart Pool | 4.5 |
| Lymph Node Pool | 8.9 |
| Fetal Skeletal Muscle | 4.0 |
| Skeletal Muscle Pool | 2.3 |
| Spleen Pool | 4.1 |
| Thymus Pool | 8.2 |
| CNS cancer (glio/astro) U87-MG | 55.5 |
| CNS cancer (glio/astro) U-118-MG | 49.7 |
| CNS cancer (neuro; met) SK-N-AS | 49.7 |
| CNS cancer (astro) SF-539 | 22.1 |
| CNS cancer (astro) SNB-75 | 45.1 |
| CNS cancer (glio) SNB-19 | 16.7 |
| CNS cancer (glio) SF-295 | 56.6 |
| Brain (Amygdala) Pool | 7.3 |
| Brain (cerebellum) | 20.0 |
| Brain (fetal) | 8.0 |
| Brain (Hippocampus) Pool | 8.1 |
| Cerebral Cortex Pool | 12.0 |
| Brain (Substantia nigra) Pool | 6.7 |
| Brain (Thalamus) Pool | 12.1 |
| Brain (whole) | 7.1 |
| Spinal Cord Pool | 6.7 |
| Adrenal Gland | 6.9 |
| Pituitary gland Pool | 2.9 |
| Salivary Gland | 2.6 |
| Thyroid (female) | 2.5 |
| Pancreatic ca. CAPAN2 | 23.3 |
| Pancreas Pool | 6.0 |

General_screening_panel_v1.6 Summary: Ag7057 Highest expression of this gene is detected in a gastric cancer cell line (CT=27). Moderate levels of expression of this gene is also seen in cluster of cancer cell lines derived from pancreatic, gastric, colon, lung, liver, renal, breast, ovarian, prostate, squamous cell carcinoma, melanoma and brain cancers. Thus, expression of this gene could be used as a marker to detect the presence of these cancers. Furthermore, therapeutic modulation of the expression or function of this gene may be effective in the treatment of pancreatic, gastric, colon, lung, liver, renal, breast, ovarian, prostate, squamous cell carcinoma, melanoma and brain cancers.

Among tissues with metabolic or endocrine function, this gene is expressed at moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

In addition, this gene is expressed at moderate levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

N. CG145494-01: Prestin.

Expression of gene CG145494-01 was assessed using the primer-probe sets Ag6694, Ag7803 and Ag7797, described in Tables NA, NB and NC. Results of the RTQ-PCR runs are shown in Table ND.

TABLE NA

Probe Name Ag6694

| | | | Start | SEQ ID |
|---|---|---|---|---|
| Primers | Sequeces | Length | Position | No |
| Forward | 5′-ggcacagaggccagagat-3′ | 18 | 559 | 300 |
| Probe | TET-5′-gtgaccttactttcaggaatcattcagttttgc-3′-TAMRA | 33 | 604 | 301 |
| Reverse | 5′-ggctctgtgaqatatatggcc-3′ | 21 | 663 | 302 |

TABLE NB

Probe Name Ag7803

| | | | Start | SEQ ID |
|---|---|---|---|---|
| Primers | Sequencs | Length | Position | No |
| Forward | 5′-ggagaaccagcaaaatagagct-3′ | 22 | 1367 | 303 |
| Probe | TET-5′-ccaatcccaggaacaaggaggacacaa-3′-TAMRA | 27 | 1409 | 304 |
| Reverse | 5′-atcacagcagtgatcaaacca-3′ | 21 | 1440 | 305 |

TABLE NC

Probe Name Ag7797

| | | | Start | SEQ ID |
|---|---|---|---|---|
| Primers | Sequenes | Length | Position | No |
| Forward | 5′-ccatctggcttaccacttttg-3′ | 21 | 1391 | 306 |
| Probe | TET-5′-cacagcagtgatcaaaccatagtccaatcc-3′-TAMRA | 30 | 1429 | 307 |
| Reverse | 5′-aaatcacagtcagcagagcaat-3′ | 22 | 1462 | 308 |

TABLE ND
General_screening_panel_v1.6

| | Rel. |
|---|---|
| | Exp. (%) |
| | Ag6694, |
| | Run |
| Tissue Name | 277223811 |
| Adipose | 0.0 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 0.0 |
| Squamous cell carcinoma SCC-4 | 0.0 |
| Testis Pool | 0.0 |
| Prostate ca.* (bone met) PC-3 | 100.0 |
| Prostate Pool | 0.9 |
| Placenta | 0.0 |
| Uterus Pool | 0.0 |
| Ovarian ca. OVCAR-3 | 0.0 |
| Ovarian ca. SK-OV-3 | 0.0 |
| Ovarian ca. OVCAR-4 | 0.0 |
| Ovarian ca. OVCAR-5 | 0.0 |
| Ovarian ca. IGROV-1 | 0.0 |
| Ovarian ca. OVCAR-8 | 0.0 |
| Ovary | 0.0 |
| Breast ca. MCF-7 | 0.0 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 0.0 |
| Trachea | 1.0 |
| Lung | 0.0 |
| Fetal Lung | 2.9 |
| Lung ca. NCI-N417 | 0.0 |
| Lung ca. LX-1 | 0.0 |
| Lung ca. NCI-H146 | 0.0 |
| Lung ca. SHP-77 | 0.0 |
| Lung ca. A549 | 0.0 |
| Lung ca. NCI-H526 | 0.0 |
| Lung ca. NCI-H23 | 0.0 |
| Lung ca. NCI-H460 | 0.0 |
| Lung ca. HOP-62 | 0.0 |
| Lung ca. NCI-H522 | 0.0 |
| Liver | 0.0 |
| Fetal Liver | 0.0 |
| Liver ca. HepG2 | 0.0 |
| Kidney Pool | 0.0 |
| Fetal Kidney | 0.0 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 0.0 |
| Bladder | 0.0 |
| Gastric ca. (liver met.) NCI-N87 | 0.0 |
| Gastric ca. KATO III | 0.0 |
| Colon ca. SW-948 | 0.0 |
| Colon ca. SW480 | 0.0 |
| Colon ca.* (SW480 met) SW620 | 0.0 |
| Colon ca. HT29 | 0.0 |
| Colon ca. HCT-116 | 0.0 |
| Colon ca. CaCo-2 | 0.0 |
| Colon cancer tissue | 0.0 |
| Colon ca. SW1116 | 0.0 |
| Colon ca. Colo-205 | 0.0 |
| Colon ca. SW-48 | 0.0 |
| Colon Pool | 0.0 |
| Small Intestine Pool | 0.0 |
| Stomach Pool | 0.0 |
| Bone Marrow Pool | 0.0 |
| Fetal Heart | 0.0 |
| Heart Pool | 0.0 |
| Lymph Node Pool | 0.0 |
| Fetal Skeletal Muscle | 0.0 |
| Skeletal Muscle Pool | 0.0 |
| Spleen Pool | 0.0 |
| Thymus Pool | 0.0 |
| CNS cancer (glio/astro) U87-MG | 0.0 |
| CNS cancer (glio/astro) U-118-MG | 0.0 |
| CNS cancer (neuro; met) SK-N-AS | 0.0 |
| CNS cancer (astro) SF-539 | 0.0 |
| CNS cancer (astro) SNB-75 | 0.0 |
| CNS cancer (glio) SNB-19 | 0.0 |
| CNS cancer (glio) SF-295 | 0.0 |
| Brain (Amygdala) Pool | 0.0 |
| Brain (cerebellum) | 14.6 |
| Brain (fetal) | 0.0 |
| Brain (Hippocampus) Pool | 0.0 |
| Cerebral Cortex Pool | 0.0 |
| Brain (Substantia nigra) Pool | 0.0 |
| Brain (Thalamus) Pool | 0.0 |
| Brain (whole) | 0.0 |
| Spinal Cord Pool | 0.0 |
| Adrenal Gland | 0.0 |
| Pituitary gland Pool | 0.0 |
| Salivary Gland | 0.0 |
| Thyroid (female) | 0.0 |
| Pancreatic ca. CAPAN2 | 0.0 |
| Pancreas Pool | 0.0 |

CNS_neurodegeneration_v1.0 Summary: Ag7797 Expression of this gene is low/undetectable (CTs>34.7) across all of the samples on this panel.

General_screening_panel_v1.6 Summary: Ag6694 Moderate level of expression of this gene is restricted to prostate cancer cell line (CT=32.6). Therefore, expression of this gene may be used to distinguish this sample from other samples in this panel and also as diagnostic marker to detect the presence of prostate cancer. In addition, therapeutic modulation of this gene may be useful in the treatment of prostate cancer.

Panel 4.1D Summary: Ag7803 Expression of this gene is low/undetectable (CTs>35) across all of the samples on this panel.

O. CG145722-01: WEE1-Like Protein Kinase.

Expression of gene CG145722-01 was assessed using the primer-probe set Ag6231, described in Table OA. Results of the RTQ-PCR runs are shown in Table OB.

TABLE OA

Probe Name Ag6231

| | | | Start | SEQ ID |
|---|---|---|---|---|
| Primers | Sequence | Length | Position | NO |
| Forward | 5′-gcttcctggctaatgagatttt-3′ | 22 | 1339 | 309 |
| Probe | TET-5′-agaggattaccggcaccttcccaaag3′-TAMRA | 26 | 1364 | 310 |
| Reverse | 5′-tgttaatcccaaggcaaatatg-3′ | 22 | 1394 | 311 |

TABLE OB
General_screening_panel_v1.5

| | Rel. |
|---|---|
| | Exp. (%) |
| | Ag6231, |
| | Run |
| Tissue Name | 259211049 |
| Adipose | 0.0 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 0.0 |
| Squamous cell carcinoma SCC-4 | 0.0 |
| Testis Pool | 0.0 |
| Prostate ca.* (bone met) PC-3 | 0.0 |
| Prostate Pool | 0.0 |
| Placenta | 0.0 |
| Uterus Pool | 0.0 |
| Ovarian ca. OVCAR-3 | 0.0 |
| Ovarian ca. SK-OV-3 | 0.0 |
| Ovarian ca. OVCAR-4 | 0.0 |
| Ovarian ca. OVCAR-5 | 0.0 |
| Ovarian ca. IGROV-1 | 0.0 |
| Ovarian ca. OVCAR-8 | 0.0 |
| Ovary | 0.0 |
| Breast ca. MCF-7 | 0.0 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 0.0 |
| Trachea | 0.0 |
| Lung | 0.0 |
| Fetal Lung | 0.0 |
| Lung ca. NCI-N417 | 0.0 |
| Lung ca. LX-1 | 0.0 |
| Lung ca. NCI-H146 | 100.0 |
| Lung ca. SHP-77 | 2.3 |
| Lung ca. A549 | 0.0 |
| Lung ca. NCI-H526 | 0.0 |
| Lung ca. NCI-H23 | 0.0 |
| Lung ca. NCI-H460 | 0.0 |
| Lung ca. HOP-62 | 0.0 |
| Lung ca. NCI-H522 | 0.0 |
| Liver | 0.0 |
| Fetal Liver | 0.0 |
| Liver ca. HepG2 | 0.0 |
| Kidney Pool | 1.8 |
| Fetal Kidney | 2.2 |
| Renal ca. 786-0 | 0.0 |
| Renal ca. A498 | 0.0 |
| Renal ca. ACHN | 0.0 |
| Renal ca. UO-31 | 6.0 |
| Renal ca. TK-10 | 0.0 |
| Bladder | 0.0 |
| Gastric ca. (liver met.) NCI-N87 | 0.0 |
| Gastric ca. KATO III | 0.0 |
| Colon ca. SW-948 | 0.0 |
| Colon ca. SW480 | 0.0 |
| Colon ca.* (SW480 met) SW620 | 0.0 |
| Colon ca. HT29 | 0.0 |
| Colon ca. HCT-116 | 0.0 |
| Colon ca. CaCo-2 | 97.3 |
| Colon cancer tissue | 0.0 |
| Colon ca. SW1116 | 0.0 |
| Colon ca. Colo-205 | 0.0 |
| Colon ca. SW-48 | 0.0 |
| Colon Pool | 0.0 |
| Small Intestine Pool | 0.0 |
| Stomach Pool | 0.0 |
| Bone Marrow Pool | 0.0 |
| Fetal Heart | 0.0 |
| Heart Pool | 0.0 |
| Lymph Node Pool | 0.0 |
| Fetal Skeletal Muscle | 0.0 |
| Skeletal Muscle Pool | 0.0 |
| Spleen Pool | 0.0 |
| Thymus Pool | 0.0 |
| CNS cancer (glio/astro) U87-MG | 0.0 |
| CNS cancer (glio/astro) U-118-MG | 0.0 |
| CNS cancer (neuro; met) SK-N-AS | 0.0 |
| CNS cancer (astro) SF-539 | 0.0 |
| CNS cancer (astro) SNB-75 | 4.2 |
| CNS cancer (glio) SNB-19 | 0.0 |
| CNS cancer (glio) SF-295 | 0.0 |
| Brain (Amygdala) Pool | 2.3 |
| Brain (cerebellum) | 5.6 |
| Brain (fetal) | 2.6 |
| Brain (Hippocampus) Pool | 0.0 |
| Cerebral Cortex Pool | 0.0 |
| Brain (Substantia nigra) Pool | 0.0 |
| Brain (Thalamus) Pool | 0.0 |
| Brain (whole) | 3.7 |
| Spinal Cord Pool | 0.0 |
| Adrenal Gland | 0.0 |
| Pituitary gland Pool | 0.0 |
| Salivary Gland | 0.0 |
| Thyroid (female) | 0.0 |
| Pancreatic ca. CAPAN2 | 4.6 |
| Pancreas Pool | 0.0 |

CNS_neurodegeneration_v1.0 Summary: Ag6231 Expression of this gene is low/undetectable (CTs>35) across all of the samples on this panel.

General_screening_panel_v1.5 Summary: Ag6231 Low levels of expression of this gene is restricted to a lung cancer and a colon cancer cell lines (CTs=32.2). Therefore, expression of this gene may be used to distinguish these cell lines from other samples in this panel and also as diagnostic marker to detect the presence of colon and lung cancers. In addition, therapeutic modulation of this gene may be useful in the treatment of these cancers.

Panel 4.1D Summary: Ag6231 Expression of this gene is low/undetectable (CTs>35) across all of the samples on this panel.

P. CG145754-02: Kallikrein 7 Precursor.

Expression of gene CG145754-02 was assessed using the primer-probe set Ag7038, described in Table PA. Results of the RTQ-PCR runs are shown in Tables PB and PC. Please note that CG145754-02 represents a full-length physical clone.

TABLE PA

Probe Name Ag7038

| | | | Start | SEQ ID |
|---|---|---|---|---|
| Primers | Sequence | Length | Position | No |
| Forward | 5′-tgttaatgacctcaagctcatctc-3′ | 24 | 342 | 312 |
| Probe | TET-5′-ccccaggactgcacgaaggtttacaa-3′-TAMRA | 26 | 367 | 313 |
| Reverse | 5′-tttcttggagtcggggatg-3′ | 19 | 426 | 314 |

TABLE PB
General_screening_panel_v1.6

| | Rel. |
|---|---|
| | Exp. (%) |
| | Ag7038, |
| | Run |
| Tissue Name | 282273672 |
| Adipose | 1.6 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 0.0 |
| Squamous cell carcinoma SCC-4 | 3.0 |
| Testis Pool | 0.0 |
| Prostate ca.* (bone met) PC-3 | 0.0 |
| Prostate Pool | 0.0 |
| Placenta | 0.0 |
| Uterus Pool | 0.0 |
| Ovarian ca. OVCAR-3 | 4.1 |
| Ovarian ca. SK-OV-3 | 0.0 |
| Ovarian ca. OVCAR-4 | 3.1 |
| Ovarian ca. OVCAR-5 | 0.0 |
| Ovarian ca. IGROV-1 | 0.0 |
| Ovarian ca. OVCAR-8 | 0.0 |
| Ovary | 0.0 |
| Breast ca. MCF-7 | 0.0 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 0.0 |
| Trachea | 0.0 |
| Lung | 0.0 |
| Fetal Lung | 0.0 |
| Lung ca. NCI-N417 | 0.0 |
| Lung ca. LX-1 | 0.5 |
| Lung ca. NCI-H146 | 0.0 |
| Lung ca. SHP-77 | 0.0 |
| Lung ca. A549 | 0.0 |
| Lung ca. NCI-H526 | 0.0 |
| Lung ca. NCI-H23 | 4.2 |
| Lung ca. NCI-H460 | 0.0 |
| Lung ca. HOP-62 | 0.0 |
| Lung ca. NCI-H522 | 0.0 |
| Liver | 0.0 |
| Fetal Liver | 0.0 |
| Liver ca. HepG2 | 0.0 |
| Kidney Pool | 0.0 |
| Fetal Kidney | 1.3 |
| Renal ca. 786-0 | 0.0 |
| Renal ca. A498 | 0.6 |
| Renal ca. ACHN | 0.0 |
| Renal ca. UO-31 | 0.0 |
| Renal ca. TK-10 | 0.0 |
| Bladder | 0.0 |
| Gastric ca. (liver met.) NCI-N87 | 100.0 |
| Gastric ca. KATO III | 22.1 |
| Colon ca. SW-948 | 4.4 |
| Colon ca. SW480 | 10.5 |
| Colon ca.* (SW480 met) SW620 | 0.0 |
| Colon ca. HT29 | 0.0 |
| Colon ca. HCT-116 | 9.7 |
| Colon ca. CaCo-2 | 0.0 |
| Colon cancer tissue | 0.6 |
| Colon ca. SW1116 | 38.7 |
| Colon ca. Colo-205 | 0.0 |
| Colon ca. SW-48 | 0.0 |
| Colon Pool | 0.0 |
| Small Intestine Pool | 0.0 |
| Stomach Pool | 0.0 |
| Bone Marrow Pool | 0.0 |
| Fetal Heart | 0.0 |
| Heart Pool | 0.0 |
| Lymph Node Pool | 0.0 |
| Fetal Skeletal Muscle | 0.0 |
| Skeletal Muscle Pool | 0.0 |
| Spleen Pool | 0.0 |
| Thymus Pool | 0.0 |
| CNS cancer (glio/astro) U87-MG | 0.0 |
| CNS cancer (glio/astro) U-118-MG | 0.0 |
| CNS cancer (neuro; met) SK-N-AS | 0.0 |
| CNS cancer (astro) SF-539 | 0.0 |
| CNS cancer (astro) SNB-75 | 2.0 |
| CNS cancer (glio) SNB-19 | 0.0 |
| CNS cancer (glio) SF-295 | 0.0 |
| Brain (Amygdala) Pool | 1.5 |
| Brain (cerebellum) | 5.6 |
| Brain (fetal) | 0.0 |
| Brain (Hippocampus) Pool | 4.0 |
| Cerebral Cortex Pool | 3.1 |
| Brain (Substantia nigra) Pool | 1.4 |
| Brain (Thalamus) Pool | 3.9 |
| Brain (whole) | 0.2 |
| Spinal Cord Pool | 0.3 |
| Adrenal Gland | 0.0 |
| Pituitary gland Pool | 0.0 |
| Salivary Gland | 0.0 |
| Thyroid (female) | 0.0 |
| Pancreatic ca. CAPAN2 | 2.2 |
| Pancreas Pool | 0.0 |

TABLE PC
Panel 5 Islet

| | Rel. |
|---|---|
| | Exp. (%) |
| | Ag703, |
| | Run |
| Tissue Name | 305424861 |
| 97457_Patient-02go_adipose | 3.0 |
| 97476_Patient-07sk_skeletal muscle | 0.0 |
| 97477_Patient-07ut_uterus | 0.0 |
| 97478_Patient-07pl_placenta | 0.0 |
| 99167_Bayer Patient 1 | 0.0 |
| 97482_Patient-08ut_uterus | 0.0 |
| 97483_Patient-08pl_placenta | 0.0 |
| 97486_Patient-09sk_skeletal muscle | 0.0 |
| 97487_Patient-09ut_uterus | 0.0 |
| 97488_Patient-09pl_placenta | 0.0 |
| 97492_Patient-10ut_uterus | 0.0 |
| 97493_Patient-10pl_placenta | 0.0 |
| 97495_Patient-11go_adipose | 2.7 |
| 97496_Patient-11sk_skeletal muscle | 0.0 |
| 97497_Patient-11ut_uterus | 0.0 |
| 97498_Patient-11pl_placenta | 0.0 |
| 97500_Patient-12go_adipose | 1.5 |
| 97501_Patient-12sk_skeletal muscle | 0.0 |
| 97502_Patient-12ut_uterus | 1.0 |
| 97503_Patient-12pl_placenta | 0.0 |
| 94721_Donor 2 U - A_Mesenchymal Stem Cells | 0.0 |
| 94722_Donor 2 U - B_Mesenchymal Stem Cells | 0.0 |
| 94723_Donor 2 U - C_Mesenchymal Stem Cells | 0.0 |
| 94709_Donor 2 AM - A_adipose | 0.0 |
| 94710_Donor 2 AM - B_adipose | 100.0 |
| 94711_Donor 2 AM - C_adipose | 0.0 |
| 94712_Donor 2 AD - A_adipose | 0.0 |
| 94713_Donor 2 AD - B_adipose | 0.0 |
| 94714_Donor 2 AD - C_adipose | 0.0 |
| 94742_Donor 3 U - A_Mesenchymal Stem Cells | 13.0 |
| 94743_Donor 3 U - B_Mesenchymal Stem Cells | 5.5 |
| 94730_Donor 3 AM - A_adipose | 0.0 |
| 94731_Donor 3 AM - B_adipose | 0.0 |
| 94732_Donor 3 AM - C_adipose | 0.0 |
| 94733_Donor 3 AD - A_adipose | 0.0 |
| 94734_Donor 3 AD - B_adipose | 0.0 |
| 94735_Donor 3 AD - C_adipose | 0.0 |
| 77138_Liver_HepG2untreated | 0.0 |
| 73556_Heart_Cardiac stromal cells (primary) | 0.0 |
| 81735_Small Intestine | 0.0 |
| 72409_Kidney_Proximal Convoluted Tubule | 2.4 |
| 82685_Small intestine_Duodenum | 0.0 |
| 90650_Adrenal_Adrenocortical adenoma | 0.0 |
| 72410_Kidney_HRCE | 5.7 |
| 72411_Kidney_HRE | 10.2 |
| 73139_Uterus_Uterine smooth muscle cells | 0.0 |

General_screening_panel_v1.6 Summary: Ag7038 Highest expression of this gene is detected in a gastric cancer NCI-N87 cell line (CT=31.3). Expression of this gene seems to be restricted to number of colon and gastric cancer cell lines. Therefore, expression of this gene may be used to distinguish colon and gastric cancer cell lines from other samples in this panel and also as a diagnostic marker to detect the presence of colon and gastric cancers. In addition, therapeutic modulation of this gene may be useful in the treatment of colon and gastric cancer.

Panel 5 Islet Summary: Ag7038 Low levels of expression of this gene is restricted to adipose tissue (CT=33). Therefore, expression of this gene may be used to distinguish this adipose sample from other samples in this panel. In addition, therapeutic modulation of this gene may be useful in the treatment of metabolic diseases such as obesity and diabetes.

Another experiment (Run 307650500) with this probe-primer set showed low/undetectable (CTs>35) across all of the samples on this panel.

Q. CG145754-03: Kallikrein-7.

Expression of gene CG145754-03 was assessed using the primer-probe set Ag5272, described in Table QA. Results of the RTQ-PCR runs are shown in Table QB.

TABLE QA

Probe Name Ag5272

| | | | Start | SEQ ID |
|---|---|---|---|---|
| Primers | Sequence | Length | Position | No |
| Forward | 5′-ggcagccaggggtgacaa-3′ | 18 | 119 | 315 |
| Probe | TET-5′-cgccccatgtgcaagaggctccc-3′-TAMRA | 23 | 149 | 316 |
| Reverse | 5′-cctccgcagtggagctgatt-3′ | 20 | 201 | 317 |

TABLE QB
Panel 4.1D

| | Rel. |
|---|---|
| | Exp. (%) |
| | Ag5272, |
| | Run |
| Tissue Name | 230500478 |
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 0.6 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 0.0 |
| EOL-1 dbcAMP | 0.0 |
| EOL-1 dbcAMP PMA/ionomycin | 0.0 |
| Dendritic cells none | 0.0 |
| Dendritic cells LPS | 0.5 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 1.3 |
| Small airway epithelium none | 100.0 |
| Small airway epithelium TNFalpha + IL-1beta | 46.7 |
| Coronery artery SMC rest | 0.0 |
| Coronery artery SMC TNFalpha + IL-1beta | 0.0 |
| Astrocytes rest | 0.0 |
| Astrocytes TNFalpha + IL-1beta | 0.0 |
| KU-812 (Basophil) rest | 0.0 |
| KU-812 (Basophil) PMA/ionomycin | 1.2 |
| CCD1106 (Keratinocytes) none | 14.2 |
| CCD1106 (Keratinocytes) TNFalpha + IL-1beta | 4.5 |
| Liver cirrhosis | 0.0 |
| NCI-H292 none | 0.0 |
| NCI-H292 IL-4 | 0.0 |
| NCI-H292 IL-9 | 0.0 |
| NCI-H292 IL-13 | 0.6 |
| NCI-H292 IFN gamma | 0.0 |
| HPAEC none | 0.0 |
| HPAEC TNF alpha + IL-1 beta | 0.0 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 0.0 |
| Dermal fibroblast IL-4 | 0.0 |
| Dermal Fibroblasts rest | 0.0 |
| Neutrophils TNFa + LPS | 0.0 |
| Neutrophils rest | 0.0 |
| Colon | 0.0 |
| Lung | 0.0 |
| Thymus | 0.0 |
| Kidney | 11.2 |

Panel 4.1D Summary: Ag5272 Highest expression of this gene is seen in resting small airway epithelium (CT=32). Significant expression of this gene is also seen in cytokines TNF-a and IL-1b treated small airway epithelium. Therefore, modulation of the expression or activity of the protein encoded by this transcript through the application of small molecule therapeutics may be useful in the treatment of asthma, COPD, and emphysema.

R. CG146279-01: Potassium Channel Subfamily K Member 10.

Expression of gene CG146279-01 was assessed using the primer-probe set Ag6035, described in Table RA. Results of the RTQ-PCR runs are shown in Tables RB, RC, RD and RE.

TABLE RA

Probe Name Ag6035

| | | | Start | SEQ ID |
|---|---|---|---|---|
| Primers | Sequence | Length | Position | No |
| Forward | 5′-atgaaatttccaatcgagacg-3′ | 21 | 61 | 318 |
| Probe | TET-5′-ctaaagtggccgttcccgcagc-3′-TAMRA | 22 | 107 | 319 |
| Reverse | 5′-ggggttgcccgttagtg-3′ | 17 | 156 | 320 |

TABLE RB
CNS_neurodegeneration_v1.0

| | Rel. |
|---|---|
| | Exp. (%) |
| | Ag6035, |
| | Run |
| Tissue Name | 225246892 |
| AD 1 Hippo | 22.5 |
| AD 2 Hippo | 25.9 |
| AD 3 Hippo | 12.4 |
| AD 4 Hippo | 13.5 |
| AD 5 Hippo | 82.9 |
| AD 6 Hippo | 74.2 |
| Control 2 Hippo | 21.5 |
| Control 4 Hippo | 19.3 |
| Control (Path) 3 Hippo | 8.2 |
| AD 1 Temporal Ctx | 24.3 |
| AD 2 Temporal Ctx | 43.8 |
| AD 3 Temporal Ctx | 4.5 |
| AD 4 Temporal Ctx | 36.6 |
| AD 5 Inf Temporal Ctx | 100.0 |
| AD 5 Sup Temporal Ctx | 62.0 |
| AD 6 Inf Temporal Ctx | 74.7 |
| AD 6 Sup Temporal Ctx | 65.1 |
| Control 1 Temporal Ctx | 5.8 |
| Control 2 Temporal Ctx | 29.5 |
| Control 3 Temporal Ctx | 22.7 |
| Control 3 Temporal Ctx | 22.7 |
| Control (Path) 1 Temporal Ctx | 74.2 |
| Control (Path) 2 Temporal Ctx | 47.0 |
| Control (Path) 3 Temporal Ctx | 9.9 |
| Control (Path) 4 Temporal Ctx | 38.2 |
| AD 1 Occipital Ctx | 22.2 |
| AD 2 Occipital Ctx (Missing) | 0.0 |
| AD 3 Occipital Ctx | 5.3 |
| AD 4 Occipital Ctx | 35.4 |
| AD 5 Occipital Ctx | 40.9 |
| AD 6 Occipital Ctx | 17.7 |
| Control 1 Occipital Ctx | 4.8 |
| Control 2 Occipital Ctx | 53.2 |
| Control 3 Occipital Ctx | 39.2 |
| Control 4 Occipital Ctx | 8.2 |
| Control (Path) 1 Occipital Ctx | 88.3 |
| Control (Path) 2 Occipital Ctx | 7.1 |
| Control (Path) 3 Occipital Ctx | 2.5 |
| Control (Path) 4 Occipital Ctx | 37.1 |
| Control 1 Parietal Ctx | 8.9 |
| Control 2 Parietal Ctx | 77.4 |
| Control 3 Parietal Ctx | 17.1 |
| Control (Path) 1 Parietal Ctx | 77.9 |
| Control (Path) 2 Parietal Ctx | 22.4 |
| Control (Path) 3 Parietal Ctx | 6.3 |
| Control (Path) 4 Parietal Ctx | 51.4 |

TABLE RC
General_screening_panel_v1.5

| | Rel. Exp. (%) |
|---|---|
| | Ag6035, Run |
| Tissue Name | 228763481 |
| Adipose | 0.5 |
| Melanoma* Hs688(A).T | 0.0 |
| Melanoma* Hs688(B).T | 0.0 |
| Melanoma* M14 | 0.0 |
| Melanoma* LOXIMVI | 0.0 |
| Melanoma* SK-MEL-5 | 0.0 |
| Squamous cell carcinoma SCC-4 | 0.0 |
| Testis Pool | 1.3 |
| Prostate ca.* (bone met) PC-3 | 0.0 |
| Prostate Pool | 4.7 |
| Placenta | 2.0 |
| Uterus Pool | 2.5 |
| Ovarian ca. OVCAR-3 | 3.3 |
| Ovarian ca. SK-OV-3 | 2.8 |
| Ovarian ca. OVCAR-4 | 3.8 |
| Ovarian ca. OVCAR-5 | 7.0 |
| Ovarian ca. IGROV-1 | 10.4 |
| Ovarian ca. OVCAR-8 | 3.1 |
| Ovary | 1.1 |
| Breast ca. MCF-7 | 3.7 |
| Breast ca. MDA-MB-231 | 6.9 |
| Breast ca. BT 549 | 2.0 |
| Breast ca. T47D | 1.1 |
| Breast ca. MDA-N | 4.3 |
| Breast Pool | 4.9 |
| Trachea | 0.2 |
| Lung | 1.1 |
| Fetal Lung | 4.1 |
| Lung ca. NCI-N417 | 3.9 |
| Lung ca. LX-1 | 30.1 |
| Lung ca. NCI-H146 | 8.4 |
| Lung ca. SHP-77 | 33.4 |
| Lung ca. A549 | 15.3 |
| Lung ca. NCI-H526 | 4.8 |
| Lung ca. NCI-H23 | 5.1 |
| Lung ca. NCI-H460 | 7.9 |
| Lung ca. HOP-62 | 0.0 |
| Lung ca. NCI-H522 | 0.0 |
| Liver | 0.5 |
| Fetal Liver | 2.0 |
| Liver ca. HepG2 | 7.4 |
| Kidney Pool | 1.6 |
| Fetal Kidney | 3.5 |
| Renal ca. 786-0 | 2.4 |
| Renal ca. A498 | 2.4 |
| Renal ca. ACHN | 11.8 |
| Renal ca.UO-31 | 6.2 |
| Renal ca. TK-10 | 9.3 |
| Bladder | 2.6 |
| Gastric ca. (liver met.) NCI-N87 | 8.2 |
| Gastric ca. KATO III | 12.8 |
| Colon ca. SW-948 | 1.0 |
| Colon ca. SW480 | 14.8 |
| Colon ca.* (SW480 met) SW620 | 29.1 |
| Colon ca. HT29 | 1.7 |
| Colon ca. HCT-116 | 12.7 |
| Colon ca. CaCo-2 | 12.3 |
| Colon cancer tissue | 5.3 |
| Colon ca. SW1116 | 0.0 |
| Colon ca. Colo-205 | 3.7 |
| Colon ca. SW-48 | 3.4 |
| Colon Pool | 0.9 |
| Small Intestine Pool | 1.5 |
| Stomach Pool | 2.1 |
| Bone Marrow Pool | 0.5 |
| Fetal Heart | 1.3 |
| Heart Pool | 0.2 |
| Lymph Node Pool | 0.9 |
| Fetal Skeletal Muscle | 1.4 |
| Skeletal Muscle Pool | 2.3 |
| Spleen Pool | 0.6 |
| Thymus Pool | 2.8 |
| CNS cancer (glio/astro) U87-MG | 0.0 |
| CNS cancer (glio/astro) U-118-MG | 2.8 |
| CNS cancer (neuro; met) SK-N-AS | 2.0 |
| CNS cancer (astro) SF-539 | 2.2 |
| CNS cancer (astro) SNB-75 | 4.6 |
| CNS cancer (glio) SNB-19 | 4.4 |
| CNS cancer (glio) SF-295 | 11.4 |
| Brain (Amygdala) Pool | 15.1 |
| Brain (cerebellum) | 100.0 |
| Brain (fetal) | 92.7 |
| Brain (Hippocampus) Pool | 32.1 |
| Cerebral Cortex Pool | 21.8 |
| Brain (Substantia nigra) Pool | 18.4 |
| Brain (Thalamus) Pool | 24.8 |
| Brain (whole) | 29.9 |
| Spinal Cord Pool | 16.3 |
| Adrenal Gland | 2.2 |
| Pituitary gland Pool | 3.7 |
| Salivary Gland | 1.0 |
| Thyroid (female) | 2.0 |
| Pancreatic ca. CAPAN2 | 0.0 |
| Pancreas Pool | 0.6 |

TABLE RD
Panel 4.1D

| | Rel. Exp. (%) |
|---|---|
| | Ag6035, Run |
| Tissue Name | 225157775 |
| Secondary Th1 act | 0.0 |
| Secondary Th2 act | 0.0 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 0.0 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 10.1 |
| Two Way MLR 5 day | 0.0 |
| Two Way MLR 7 day | 0.0 |
| PBMC rest | 5.5 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 0.0 |
| EOL-1 dbcAMP | 100.0 |
| EOL-1 dbcAMP PMA/ionomycin | 36.1 |
| Dendritic cells none | 0.0 |
| Dendritic cells LPS | 0.0 |
| Dendritic cells anti-CD40 | 0.0 |
| Monocytes rest | 9.9 |
| Monocytes LPS | 0.0 |
| Macrophages rest | 0.0 |
| Macrophages LPS | 0.0 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + IL-1beta | 0.0 |
| Bronchial epithelium TNFalpha + IL1beta | 0.0 |
| Small airway epithelium none | 0.0 |
| Small airway epithelium TNFalpha + IL-1beta | 0.0 |
| Coronery artery SMC rest | 0.0 |
| Coronery artery SMC TNFalpha + IL-1beta | 0.0 |
| Astrocytes rest | 0.0 |
| Astrocytes TNFalpha + IL-1beta | 0.0 |
| KU-812 (Basophil) rest | 0.0 |
| KU-812 (Basophil) PMA/ionomycin | 0.0 |
| CCD1106 (Keratinocytes) none | 0.0 |
| CCD1106 (Keratinocytes) TNFalpha + IL-1beta | 0.0 |
| Liver cirrhosis | 0.0 |
| NCI-H292 none | 0.0 |
| NCI-H292 IL-4 | 0.0 |
| NCI-H292 IL-9 | 0.0 |
| NCI-H292 IL-13 | 0.0 |
| NCI-H292 IFN gamma | 0.0 |
| HPAEC none | 0.0 |
| HPAEC TNF alpha + IL-1 beta | 0.0 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 0.0 |
| Dermal fibroblast IL-4 | 0.0 |
| Dermal Fibroblasts rest | 0.0 |
| Neutrophils TNFa + LPS | 0.0 |
| Neutrophils rest | 0.0 |
| Colon | 0.0 |
| Lung | 0.0 |
| Thymus | 8.5 |
| Kidney | 7.7 |

TABLE RE
Panel 5 Islet

| | Rel. Exp. (%) | Rel. Exp. (%) |
|---|---|---|
| | Ag6035 Run | Ag6035, Run |
| Tissue Name | 253578284 | 306414003 |
| 97457_Patient-02go_adipose | 0.0 | 0.0 |
| 97476_Patient-07sk_skeletal | 0.0 | 0.0 |
| muscle | | |
| 97477_Patient-07ut_uterus | 0.0 | 0.0 |
| 97478_Patient-07pl_placenta | 0.0 | 0.0 |
| 99167_Bayer Patient 1 | 100.0 | 100.0 |
| 97482_Patient-08ut_uterus | 0.0 | 0.0 |
| 97483_Patient-08pl_placenta | 0.0 | 0.0 |
| 97486_Patient-09sk_skeletal | 0.0 | 0.0 |
| muscle | | |
| 97487_Patient-09ut_uterus | 0.0 | 0.0 |
| 97488_Patient-09pl_placenta | 0.0 | 0.0 |
| 97492_Patient-10ut_uterus | 0.0 | 0.0 |
| 97493_Patient-10pl_placenta | 0.0 | 0.0 |
| 97495_Patient-11go_adipose | 0.0 | 0.0 |
| 97496_Patient-11sk_skeletal | 0.0 | 0.0 |
| muscle | | |
| 97497_Patient-11ut_uterus | 0.0 | 0.0 |
| 97498_Patient-11pl_placenta | 0.0 | 0.0 |
| 97500_Patient-12go_adipose | 0.0 | 0.0 |
| 97501_Patient-12sk_skeletal | 0.0 | 0.0 |
| muscle | | |
| 97502_Patient-12ut_uterus | 0.0 | 0.0 |
| 97503_Patient-12pl_placenta | 0.0 | 0.0 |
| 94721_Donor 2 U - A_Mesenchymal | 0.0 | 0.0 |
| Stem Cells | | |
| 94722_Donor 2 U - B_Mesenchymal | 0.0 | 0.0 |
| Stem Cells | | |
| 94723_Donor 2 U - C_Mesenchymal | 0.0 | 0.0 |
| Stem Cells | | |
| 94709_Donor2 AM - A_adipose | 0.0 | 0.0 |
| 94710_Donor2 AM - B_adipose | 0.0 | 0.0 |
| 94711_Donor2 AM - C_adipose | 0.0 | 0.0 |
| 94712_Donor2 AD - A_adipose | 0.0 | 0.0 |
| 94713_Donor2 AD - B_adipose | 0.0 | 0.0 |
| 94714_Donor2 AD - C_adipose | 0.0 | 0.0 |
| 94742_Donor 3 U - A_Mesenchymal | 0.0 | 0.0 |
| Stem Cells | | |
| 94743_Donor 3 U - B_Mesenchymal | 0.0 | 0.0 |
| Stem Cells | | |
| 94730_Donor 3 AM - A_adipose | 0.0 | 0.0 |
| 94731_Donor 3 AM - B_adipose | 0.0 | 0.0 |
| 94732_Donor 3 AM - C_adipose | 0.0 | 0.0 |
| 94733_Donor 3 AD - A_adipose | 0.0 | 0.0 |
| 94734_Donor 3 AD - B_adipose | 0.0 | 0.0 |
| 94735_Donor 3 AD - C_adipose | 0.0 | 0.0 |
| 77138_Liver_HepG2untreated | 0.0 | 0.0 |
| 73556_Heart_Cardiac stromal | 0.0 | 0.0 |
| cells (primary) | | |
| 81735_Small Intestine | 0.0 | 0.0 |
| 72409_Kidney_Proximal Convoluted | 0.0 | 0.0 |
| Tubule | | |
| 82685_Small intestine_Duodenum | 0.0 | 0.0 |
| 90650_Adrenal_Adrenocortical | 0.0 | 16.2 |
| adenoma | | |
| 72410_Kidney_HRCE | 0.0 | 0.0 |
| 72411_Kidney_HRE | 0.0 | 0.0 |
| 73139_Uterus_Uterine smooth | 0.0 | 0.0 |
| muscle cells | | |

CNS_neurodegeneration_v1.0 Summary: Ag6035 This panel confirms the expression of this gene at low levels in the brains of an independent group of individuals. However, no differential expression of this gene was detected between Alzheimer's diseased postmortem brains and those of non-demented controls in this experiment. Please see Panel 1.5 for a discussion of this gene in treatment of central nervous system disorders.

General_screening_panel_v1.5 Summary: Ag6035 Highest expression of this gene is detected in cerebellum (CT=27). This gene codes for a splice variant of potassium channel TREK2. As reported in literature (Bang et al., 2000, J Biol Chem 275(23): 17412-9, PMID: 10747911), this gene shows expression preferentially in all the regions of brain. Therefore, therapeutic modulation of this gene product may be useful in the treatment of central nervous system disorders such as Alzheimer's disease, Parkinson's disease, epilepsy, multiple sclerosis, schizophrenia and depression.

Moderate to low levels of expression of this gene is also seen in number of cancer cell lines derived from brain, colon, gastric, renal, lung, breast and ovarian cancer. Therefore, therapeutic modulation of this gene may be useful in the treatment of these cancers.

In addition, low levels of expression of this gene is also seen in tissues with metabolic/endocrine functions, including pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

Panel 4.1D Summary: Ag6035 Highest expression of this gene is detected in eosinophils (CT=32.5). Low levels of expression of this gene is also seen in PMA/ionomycin treated eosinophils. Therefore, therapeutic modulation of this gene or its protein product may useful in the treatment of hematopoietic disorders involving eosinophils, parasitic infections, autoimmune and inflammatory diseases including allergy and asthma.

Panel 5 Islet Summary: Ag6035 Two experiments with same probe-primer sets are in excellent agreement. Low levels of expression of this gene are restricted to islet cells (CTs=33-34). This gene codes for a splice variant of potassium channel TREK2. Potassium channels play an important role in insulin secretion by islet beta cells upon stimulation by glucose. Alteration in the insulin secretion pathway through the use of sulfonylureas or genetic inactivation of K(ATP) channels may lead to inappropriate insulin secretion at low glucose (Henquin JC., 2000, Diabetes 49(11):1751-60, PMID: 11078440). Therefore, therapeutic modulation of this gene or its protein product may be useful in the treatment type 2 diabetes.

S. CG146403-01: Diacylglycerol Acyltransferase 2.

Expression of gene CG146403-01 was assessed using the primer-probe set Ag6034, described in Table SA. Results of the RTQ-PCR runs are shown in Tables SB, SC and SD.

TABLE SA

Probe Name Ag6034

| | | | Start | SEQ ID |
|---|---|---|---|---|
| Primers | Sequence | Length | Position | No |
| Forward | 5′-tggggagaatgacatctttaga-3′ | 22 | 540 | 321 |
| Probe | TET-5′-cttaaggcttttgccacaggctcctg-3′-TAMRA | 26 | 562 | 322 |
| Reverse | 5′-agagaagcccatgagcttctt-3′ | 21 | 613 | 323 |

TABLE SB
General_screening_panel_v1.5

| | Rel. Exp. (%) |
|---|---|
| | Ag6034, Run |
| Tissue Name | 228763480 |
| Adipose | 0.2 |
| Melanoma* Hs688(A).T | 0.0 |
| Melanoma* Hs688(B).T | 0.0 |
| Melanoma* M14 | 0.1 |
| Melanoma* LOXIMVI | 0.0 |
| Melanoma* SK-MEL-5 | 0.2 |
| Squamous cell carcinoma SCC-4 | 0.0 |
| Testis Pool | 0.2 |
| Prostate ca.* (bone met) PC-3 | 0.4 |
| Prostate Pool | 0.0 |
| Placenta | 0.0 |
| Uterus Pool | 0.0 |
| Ovarian ca. OVCAR-3 | 0.0 |
| Ovarian ca. SK-OV-3 | 0.2 |
| Ovarian ca. OVCAR-4 | 0.0 |
| Ovarian ca. OVCAR-5 | 0.1 |
| Ovarian ca. IGROV-1 | 0.1 |
| Ovarian ca. OVCAR-8 | 0.2 |
| Ovary | 0.0 |
| Breast ca. MCF-7 | 0.0 |
| Breast ca. MDA-MB-231 | 0.0 |
| Breast ca. BT 549 | 0.1 |
| Breast ca. T47D | 0.0 |
| Breast ca. MDA-N | 0.0 |
| Breast Pool | 0.0 |
| Trachea | 0.0 |
| Lung | 0.0 |
| Fetal Lung | 0.4 |
| Lung ca. NCI-N417 | 0.1 |
| Lung ca. LX-1 | 28.1 |
| Lung ca. NCI-H146 | 0.0 |
| Lung ca. SHP-77 | 0.0 |
| Lung ca. A549 | 0.7 |
| Lung ca. NCI-H526 | 0.0 |
| Lung ca. NCI-H23 | 0.0 |
| Lung ca. NCI-H460 | 4.2 |
| Lung ca. HOP-62 | 0.0 |
| Lung ca. NCI-H522 | 0.2 |
| Liver | 1.7 |
| Fetal Liver | 55.9 |
| Liver ca. HepG2 | 62.9 |
| Kidney Pool | 0.0 |
| Fetal Kidney | 5.1 |
| Renal ca. 786-0 | 0.0 |
| Renal ca. A498 | 0.1 |
| Renal ca. ACHN | 0.0 |
| Renal ca. UO-31 | 0.0 |
| Renal ca. TK-10 | 27.9 |
| Bladder | 1.2 |
| Gastric ca. (liver met.) NCI-N87 | 0.5 |
| Gastric ca. KATO III | 7.9 |
| Colon ca. SW-948 | 3.6 |
| Colon ca. SW480 | 12.5 |
| Colon ca.* (SW480 met) SW620 | 1.9 |
| Colon ca. HT29 | 22.7 |
| Colon ca. HCT-116 | 0.0 |
| Colon ca. CaCo-2 | 100.0 |
| Colon cancer tissue | 63.3 |
| Colon ca. SW1116 | 0.0 |
| Colon ca. Colo-205 | 2.1 |
| Colon ca. SW-48 | 50.0 |
| Colon Pool | 0.2 |
| Small Intestine Pool | 0.4 |
| Stomach Pool | 0.0 |
| Bone Marrow Pool | 0.0 |
| Fetal Heart | 0.2 |
| Heart Pool | 0.1 |
| Lymph Node Pool | 0.0 |
| Fetal Skeletal Muscle | 0.1 |
| Skeletal Muscle Pool | 0.0 |
| Spleen Pool | 0.0 |
| Thymus Pool | 0.1 |
| CNS cancer (glio/astro) U87-MG | 0.0 |
| CNS cancer (glio/astro) U-118-MG | 0.0 |
| CNS cancer (neuro; met) SK-N-AS | 0.0 |
| CNS cancer (astro) SF-539 | 0.2 |
| CNS cancer (astro) SNB-75 | 0.0 |
| CNS cancer (glio) SNB-19 | 0.0 |
| CNS cancer (glio) SF-295 | 0.0 |
| Brain (Amygdala) Pool | 0.0 |
| Brain (cerebellum) | 0.1 |
| Brain (fetal) | 0.2 |
| Brain (Hippocampus) Pool | 0.0 |
| Cerebral Cortex Pool | 0.1 |
| Brain (Substantia nigra) Pool | 0.0 |
| Brain (Thalamus) Pool | 0.0 |
| Brain (whole) | 1.1 |
| Spinal Cord Pool | 0.0 |
| Adrenal Gland | 0.0 |
| Pituitary gland Pool | 0.0 |
| Salivary Gland | 0.0 |
| Thyroid (female) | 0.0 |
| Pancreatic ca. CAPAN2 | 0.0 |
| Pancreas Pool | 0.0 |

TABLE SC
Panel 4.1D

| | Rel. Exp. (%) |
|---|---|
| | Ag6034, Run |
| Tissue Name | 225245213 |
| Secondary Th1 act | 0.0 |
| Secondary Th2 act | 0.0 |
| Secondary Tr1 act | 0.4 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 0.0 |
| Primary Tr1 rest | 0.0 |
| CD45RA CD4 lymphocyte act | 0.0 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 0.0 |
| Two Way MLR 3 day | 0.0 |
| Two Way MLR 5 day | 0.0 |
| Two Way MLR 7 day | 0.0 |
| PBMC rest | 0.0 |
| PBMC PWM | 0.9 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 0.0 |
| EOL-1 dbcAMP | 0.0 |
| EOL-1 dbcAMP PMA/ionomycin | 0.0 |
| Dendritic cells none | 0.0 |
| Dendritic cells LPS | 0.0 |
| Dendritic cells anti-CD40 | 0.5 |
| Monocytes rest | 0.0 |
| Monocytes LPS | 0.0 |
| Macrophages rest | 0.0 |
| Macrophages LPS | 0.0 |
| HUVEC none | 0.0 |
| HUVEC starved | 0.0 |
| HUVEC IL-1beta | 0.0 |
| HUVEC IFN gamma | 0.0 |
| HUVEC TNF alpha + IFN gamma | 0.0 |
| HUVEC TNF alpha + IL4 | 0.0 |
| HUVEC IL-11 | 0.0 |
| Lung Microvascular EC none | 0.0 |
| Lung Microvascular EC TNFalpha + IL-1beta | 0.0 |
| Microvascular Dermal EC none | 0.0 |
| Microsvasular Dermal EC TNFalpha + | 0.0 |
| IL-1beta | |
| Bronchial epithelium TNFalpha + IL1beta | 0.0 |
| Small airway epithelium none | 0.0 |
| Small airway epithelium TNFalpha + | 0.0 |
| IL-1beta | |
| Coronery artery SMC rest | 0.0 |
| Coronery artery SMC TNFalpha + IL-1beta | 0.6 |
| Astrocytes rest | 0.0 |
| Astrocytes TNFalpha + IL-1beta | 0.0 |
| KU-812 (Basophil) rest | 0.0 |
| KU-812 (Basophil) PMA/ionomycin | 0.0 |
| CCD1106 (Keratinocytes) none | 0.0 |
| CCD1106 (Keratinocytes) TNFalpha + | 0.0 |
| IL-1beta | |
| Liver cirrhosis | 17.0 |
| NCI-H292 none | 0.0 |
| NCI-H292 IL-4 | 0.0 |
| NCI-H292 IL-9 | 0.0 |
| NCI-H292 IL-13 | 0.0 |
| NCI-H292 IFN gamma | 0.0 |
| HPAEC none | 0.0 |
| HPAEC TNF alpha + IL-1 beta | 0.0 |
| Lung fibroblast none | 0.0 |
| Lung fibroblast TNF alpha + IL-1 beta | 0.0 |
| Lung fibroblast IL-4 | 0.0 |
| Lung fibroblast IL-9 | 0.0 |
| Lung fibroblast IL-13 | 0.0 |
| Lung fibroblast IFN gamma | 0.0 |
| Dermal fibroblast CCD1070 rest | 0.0 |
| Dermal fibroblast CCD1070 TNF alpha | 0.0 |
| Dermal fibroblast CCD1070 IL-1 beta | 0.0 |
| Dermal fibroblast IFN gamma | 0.0 |
| Dermal fibroblast IL-4 | 0.0 |
| Dermal Fibroblasts rest | 0.3 |
| Neutrophils TNFa + LPS | 4.0 |
| Neutrophils rest | 0.0 |
| Colon | 81.2 |
| Lung | 4.7 |
| Thymus | 18.0 |
| Kidney | 100.0 |

TABLE SD
Panel 5 Islet

| | Rel. Exp. (%) |
|---|---|
| | Ag603, Run |
| Tissue Name | 256791126 |
| 97457_Patient-02go_adipose | 0.0 |
| 97476_Patient-07sk_skeletal muscle | 0.0 |
| 97477_Patient-07ut_uterus | 0.0 |
| 97478_Patient-07pl_placenta | 0.0 |
| 99167_Bayer Patient 1 | 0.0 |
| 97482_Patient-08ut_uterus | 0.0 |
| 97483_Patient-08pl_placenta | 0.0 |
| 97486_Patient-09sk_skeletal muscle | 0.0 |
| 97487_Patient-09ut_uterus | 0.0 |
| 97488_Patient-09pl_placenta | 0.0 |
| 97492_Patient-10ut_uterus | 0.0 |
| 97493_Patient-10pl_placenta | 0.0 |
| 97495_Patient-11go_adipose | 0.0 |
| 97496_Patient-11sk_skeletal muscle | 0.0 |
| 97497_Patient-11ut_uterus | 0.0 |
| 97498_Patient-11pl_placenta | 0.0 |
| 97500_Patient-12go_adipose | 0.0 |
| 97501_Patient-12sk_skeletal muscle | 0.0 |
| 97502_Patient-12ut_uterus | 0.0 |
| 97503_Patient-12pl_placenta | 0.0 |
| 94721_Donor 2 U-A_Mesenchymal Stem Cells | 0.0 |
| 94722_Donor 2 U-B_Mesenchymal Stem Cells | 0.0 |
| 94723_Donor 2 U-C_Mesenchymal Stem Cells | 0.0 |
| 94709_Donor 2 AM - A_adipose | 0.0 |
| 94710_Donor 2 AM - B_adipose | 0.0 |
| 94711_Donor 2 AM - C_adipose | 0.0 |
| 94712_Donor 2 AD - A_adipose | 0.0 |
| 94713_Donor 2 AD - B_adipose | 0.0 |
| 94714_Donor 2 AD - C_adipose | 0.0 |
| 94742_Donor 3 U - A_Mesenchymal Stem Cells | 0.0 |
| 94743_Donor 3 U - B_Mesenchymal Stem Cells | 0.0 |
| 94730_Donor 3 AM - A_adipose | 0.0 |
| 94731_Donor 3 AM - B_adipose | 0.0 |
| 94732_Donor 3 AM - C_adipose | 0.0 |
| 94733_Donor 3 AD - A_adipose | 0.0 |
| 94734_Donor 3 AD - B_adipose | 0.0 |
| 94735_Donor 3 AD - C_adipose | 0.0 |
| 77138_Liver_HepG2untreated | 100.0 |
| 73556_Heart_Cardiac stromal cells (primary) | 0.0 |
| 81735_Small Intestine | 25.5 |
| 72409_Kidney_Proximal Convoluted Tubule | 0.0 |
| 82685_Small intestine_Duodenum | 31.2 |
| 90650_Adrenal_Adrenocortical adenoma | 0.0 |
| 72410_Kidney_HRCE | 0.0 |
| 72411_Kidney_HRE | 0.0 |
| 73139_Uterus_Uterine smooth muscle cells | 0.0 |

CNS_neurodegeneration_v1.0 Summary: Ag6034 Expression of this gene is low/undetectable in all samples on this panel (CTs>35). (Data not shown.)

General_screening_panel_v1.5 Summary: Ag6034 Highest expression of this gene is seen in colon cancer (CT=26.3). High to moderate levels of expression are also seen in colon, renal, liver and lung cancer cell lines, as well as in fetal lung. This expression suggests that this gene may be involved in these cancers. Thus, expression of this gene could be used to differentiate between these samples and other samples on this panel and as a marker of these cancers. Therapeutic modulation of the expression or function of this gene may also be useful in the treatment of these cancers.

Panel 4.1D Summary: Ag6034 Expression of this gene is highest in colon and kidney (CTs=30). Thus, expression of this gene could be used as a marker of these tissues.

Panel 5 Islet Summary: Ag6034 Highest expression of this gene is seen in a liver cell line (CT=30.6). Thus, expression of this gene could be used to differentiate between this sample and other samples on this panel.

T. CG146513-01: Diacylglycerol Acyltransferase 2.

Expression of gene CG146513-01 was assessed using the primer-probe set Ag6036, described in Table TA. Results of the RTQ-PCR runs are shown in Table TB.

TABLE TA

Probe Name Ag6036

| | | | Start | SEQ ID |
|---|---|---|---|---|
| Primers | Sequence | Length | Position | No |
| Forward | 5′-tggaccctatggaagtatttcc-3′ | 22 | 326 | 324 |
| Probe | TET-5′-ttcccagtacagctggtgaagactca-3′-TAMRA | 26 | 356 | 325 |
| Reverse | 5′-gttgtgtttgggagaaagatca-3′ | 22 | 382 | 326 |

TABLE TB
Panel 5 Islet

| | Rel. Exp. (%) |
|---|---|
| | Ag603, Run |
| Tissue Name | 279370869 |
| 97457_Patient-02go_adipose | 10.5 |
| 97476_Patient-07sk_skeletal muscle | 0.0 |
| 97477_Patient-07ut_uterus | 3.3 |
| 97478_Patient-07pl_placenta | 6.0 |
| 99167_Bayer Patient 1 | 3.3 |
| 97482_Patient-08ut_uterus | 2.6 |
| 97483_Patient-08pl_placenta | 1.0 |
| 97486_Patient-09sk_skeletal muscle | 8.4 |
| 97487_Patient-09ut_uterus | 5.8 |
| 97488_Patient-09pl_placenta | 2.2 |
| 97492_Patient-10ut_uterus | 4.0 |
| 97493_Patient-10pl_placenta | 3.2 |
| 97495_Patient-11go_adipose | 6.0 |
| 97496_Patient-11sk_skeletal muscle | 20.2 |
| 97497_Patient-11ut_uterus | 8.7 |
| 97498_Patient-11pl_placenta | 1.9 |
| 97500_Patient-12go_adipose | 4.0 |
| 97501_Patient-12sk_skeletal muscle | 22.2 |
| 97502_Patient-12ut_uterus | 7.1 |
| 97503_Patient-12pl_placenta | 1.3 |
| 94721_Donor 2 U - A_Mesenchymal Stem Cells | 12.8 |
| 94722_Donor 2 U - B_Mesenchymal Stem Cells | 6.8 |
| 94723_Donor 2 U - C_Mesenchymal Stem Cells | 11.2 |
| 94709_Donor 2 AM - A_adipose | 11.4 |
| 94710_Donor 2 AM - B_adipose | 6.7 |
| 94711_Donor 2 AM - C_adipose | 4.2 |
| 94712_Donor 2 AD - A_adipose | 23.8 |
| 94713_Donor 2 AD - B_adipose | 32.8 |
| 94714_Donor 2 AD - C_adipose | 22.2 |
| 94742_Donor 3 U - A_Mesenchymal Stem Cells | 2.6 |
| 94743_Donor 3 U - B_Mesenchymal Stem Cells | 2.5 |
| 94730_Donor 3 AM - A_adipose | 12.9 |
| 94731_Donor 3 AM - B_adipose | 21.0 |
| 94732_Donor 3 AM - C_adipose | 20.4 |
| 94733_Donor 3 AD - A_adipose | 26.4 |
| 94734_Donor 3 AD - B_adipose | 25.5 |
| 94735_Donor 3 AD - C_adipose | 6.5 |
| 77138_Liver_HepG2untreated | 41.5 |
| 73556_Heart_Cardiac stromal cells (primary) | 1.6 |
| 81735_Small Intestine | 10.7 |
| 72409_Kidney_Proximal Convoluted Tubule | 100.0 |
| 82685_Small intestine_Duodenum | 15.7 |
| 90650_Adrenal_Adrenocortical adenoma | 5.0 |
| 72410_Kidney_HRCE | 31.2 |
| 72411_Kidney_HRE | 9.1 |
| 73139_Uterus_Uterine smooth muscle cells | 13.3 |

CNS_neurodegeneration_v1.0 Summary: Ag6036 Expression of this gene is low/undetectable in all samples on this panel (CTs>35).

General_screen_panel_v1.5 Summary: Ag6036 Expression of this gene is low/undetectable in all samples on this panel (CTs>35).

Panel 4.1D Summary: Ag6036 Expression of this gene is low/undetectable in all samples on this panel (CTs>35).

Panel 5 Islet Summary: Ag6036 Highest expression of this gene is seen in a kidney derived sample (CT=29.5). Moderate levels of expression are seen in many samples on this panel, including samples from uterus, placenta, adipose, and skeletal muscle. Thus, this gene may be involved in diseases of these tissues, including obesity and diabetes.

U. CG146522-01: Diacylglycerol Acyltransferase 2.

Expression of gene CG146522-01 was assessed using the primer-probe set Ag6037, described in Table UA. Results of the RTQ-PCR runs are shown in Table UB.

TABLE UA

Probe Name Ag6037

| | | | | |
|---|---|---|---|---|
| | | | Start | SEQ ID |
| Primers | Sequence | Length | Position | No |

| | | | | |
|---|---|---|---|---|
| Forward | 5′-attccaagcagcctagtcactt-3′ | 22 | 49 | 327 |
| Probe | TET-5′-ttctgcagtggcctttgagctacctt-3′-TAMRA | 26 | 85 | 328 |
| Reverse | 5′-cagcaggtagacgaacaagatg-3′ | 122 | 113 | 329 |

TABLE UB
Panel 5 Islet

| | Rel. Exp.(%) |
|---|---|
| | Ag6037, Run |
| Tissue Name | 279370870 |
| 97457_Patient-02go_adipose | 0.0 |
| 97476_Patient-07sk_skeletal muscle | 0.0 |
| 97477_Patient-07ut_uterus | 0.0 |
| 97478_Patient-07pl_placenta | 0.0 |
| 99167_Bayer Patient 1 | 0.9 |
| 97482_Patient-08ut_uterus | 0.8 |
| 97483_Patient-08pl_placenta | 0.0 |
| 97486_Patient-09sk_skeletal muscle | 9.0 |
| 97487_Patient-09ut_uterus | 2.2 |
| 97488_Patient-09pl_placenta | 0.0 |
| 97492_Patient-10ut_uterus | 0.5 |
| 97493_Patient-10pl_placenta | 3.5 |
| 97495_Patient-11go_adipose | 1.2 |
| 97496_Patient-11sk_skeletal muscle | 39.2 |
| 97497_Patient-11ut_uterus | 0.0 |
| 97498_Patient-11pl_placenta | 0.0 |
| 97500_Patient-12go_adipose | 1.7 |
| 97501_Patient-12sk_skeletal muscle | 100.0 |
| 97502_Patient-12ut_uterus | 0.0 |
| 97503_Patient-12pl_placenta | 1.0 |
| 94721_Donor 2 U-A_Mesenchymal Stem Cells | 0.0 |
| 94722_Donor 2 U-B_Mesenchymal Stem Cells | 0.0 |
| 94723_Donor 2 U-C_Mesenchymal Stem Cells | 0.5 |
| 94709_Donor 2 AM - A_adipose | 0.0 |
| 94710_Donor 2 AM - B_adipose | 0.0 |
| 94711_Donor 2 AM - C_adipose | 0.0 |
| 94712_Donor 2 AD - A_adipose | 0.0 |
| 94713_Donor 2 AD - B_adipose | 0.0 |
| 94714_Donor 2 AD - C_adipose | 0.0 |
| 94742_Donor 3 U - A_Mesenchymal Stem Cells | 0.0 |
| 94743_Donor 3 U - B_Mesenchymal Stem Cells | 0.0 |
| 94730_Donor 3 AM - A_adipose | 0.0 |
| 94731_Donor 3 AM - B_adipose | 0.0 |
| 94732_Donor 3 AM - C_adipose | 0.0 |
| 94733_Donor 3 AD - A_adipose | 0.0 |
| 94734_Donor 3 AD - B_adipose | 0.9 |
| 94735_Donor 3 AD - C_adipose | 0.0 |
| 77138_Liver_HepG2untreated | 0.0 |
| 73556_Heart_Cardiac stromal cells (primary) | 0.0 |
| 81735_Small Intestine | 1.0 |
| 72409_Kidney_Proximal Convoluted Tubule | 0.0 |
| 82685_Small intestine_Duodenum | 0.0 |
| 90650_Adrenal_Adrenocortical adenoma | 0.0 |
| 72410_Kidney_HRCE | 0.0 |
| 72411_Kidney_HRE | 0.0 |
| 73139_Uterus_Uterine smooth muscle cells | 0.0 |

CNS neurodegeneration_v1.0 Summary: Ag6037 Expression of this gene is low/undetectable in all samples on this panel (CTs>35).

General_screenin_panel_v1.5 Summary: Ag6037 Expression of this gene is low/undetectable in all samples on this panel (CTs>35).

Panel 4.1D Summary: Ag6037 Expression of this gene is low/undetectable in all samples on this panel (CTs>35).

Panel 5 Islet Summary: Ag6037 Expression of this gene is limited to skeletal muscle (CTs=30-31). Thus, expression of this gene could be used to differentiate these samples from other samples on this panel and as a marker of this tissue. Furthermore, therapeutic modulation of the expression or function of this gene may be useful in the treatment of metabolic disorders, including obesity and diabetes.

V. CG146531-01: Diacylglycerol Acyltransferase 2.

Expression of gene CG146531-01 was assessed using the primer-probe set Ag6038, described in Table VA.

TABLE VA

Probe Name Ag6038

| | | | Start | SEQ ID |
|---|---|---|---|---|
| Primers | Sequence | Length | Position | No |
| Forward | 5′-aaggtgtcacaggaagagcat-3′ | 21 | 10 | 330 |
| Probe | TET-5′-agccaggtcaccatggctttcttct-3′-TAMRA | 25 | 49 | 331 |
| Reverse | 5′-gccctcctggagattcagt-3′ | 19 | 78 | 332 |

CNS_neurodegeneration_v1.0 Summary: Ag6038 Expression of this gene is low/undetectable in all samples on this panel (CTs>35).

General_screen_panel_v1.5 Summary: Ag6038 Expression of this gene is low/undetectable in all samples on this panel (CTs>35).

Panel 4.1D Summary: Ag6038 Expression of this gene is low/undetectable in all samples on this panel (CTs>35).

Panel-5 Islet Summary: Ag6038 Expression of this gene is low/undetectable in all samples on this panel (CTs>35).

W. CG147274-01: Protease.

Expression of gene CG147274-01 was assessed using the primer-probe set Ag5623, described in Table WA.

TABLE WA

Probe Name Ag5623

| | | | | |
|---|---|---|---|---|
| | | | Start | SEQ ID |
| Primers | Sequence | Length | Position | No |

| | | | | |
|---|---|---|---|---|
| Forward | 5′-gatgtgctgccttcagaatg-3′ | 20 | 64 | 333 |
| Probe | TET-5′-aatcctcccggcctccttggagt-3′-TAMRA | 23 | 89 | 334 |
| Reverse | 5′-gtccttcctgggtgtcttg-3′ | 19 | 121 | 335 |

CNS_neurodegeneration_v1.0 Summary: Ag5623 Expression of this gene is low/undetectable in all samples on this panel (CTs>35).

General_screen_panel_v1.5 Summary: Ag5623 Expression of this gene is low/undetectable in all samples on this panel (CTs>35).

Panel 4.1D Summary: Ag5623 Expression of this gene is low/undetectable in all samples on this panel (CTs>35).

X. CG147419-01: Glutamine: Fructose-6-Phosphate Amidotrancferase 1 Muscle.

Expression of gene CG147419-01 was assessed using the primer-probe set Ag5207, described in Table XA. Results of the RTQ-PCR runs are shown in Tables XB, XC, XD and XE.

TABLE XA

Probe Name Ag5207

| | | | Start | SEQ ID |
|---|---|---|---|---|
| Primers | Sequenes | Length | Position | No |
| Forward | 5′-gccctctgttgattggtgta-3′ | 20 | 736 | 336 |
| Probe | TET-5′-cggagtgaacataaactttctactgatca-3′-TAMRA | 29 | 756 | 337 |
| Reverse | 5′-ccaatctgagtcctagctgttc-3′ | 22 | 802 | 338 |

TABLE XB
CNS_neurodegeneration_v1.0

| | Rel. Exp. (%) |
|---|---|
| | Ag5207, Run |
| Tissue Name | 226559656 |
| AD 1 Hippo | 11.3 |
| AD 2 Hippo | 14.6 |
| AD 3 Hippo | 0.0 |
| AD 4 Hippo | 6.3 |
| AD 5 hippo | 100.0 |
| AD 6 Hippo | 29.3 |
| Control 2 Hippo | 59.0 |
| Control 4 Hippo | 0.0 |
| Control (Path) 3 Hippo | 1.8 |
| AD 1 Temporal Ctx | 12.5 |
| AD 2 Temporal Ctx | 41.5 |
| AD 3 Temporal Ctx | 2.2 |
| AD 4 Temporal Ctx | 24.1 |
| AD 5 Inf Temporal Ctx | 65.5 |
| AD 5 SupTemporal Ctx | 29.1 |
| AD 6 Inf Temporal Ctx | 26.2 |
| AD 6 Sup Temporal Ctx | 49.3 |
| Control 1 Temporal Ctx | 0.0 |
| Control 2 Temporal Ctx | 88.3 |
| Control 3 Temporal Ctx | 19.5 |
| Control 4 Temporal Ctx | 4.9 |
| Control (Path) 1 Temporal Ctx | 97.3 |
| Control (Path) 2 Temporal Ctx | 48.0 |
| Control (Path) 3 Temporal Ctx | 2.3 |
| Control (Path) 4 Temporal Ctx | 54.7 |
| AD 1 Occipital Ctx | 1.8 |
| AD 2 Occipital Ctx (Missing) | 0.0 |
| AD 3 Occipital Ctx | 1.7 |
| AD 4 Occipital Ctx | 11.5 |
| AD 5 Occipital Ctx | 21.0 |
| AD 6 Occipital Ctx | 97.9 |
| Control 1 Occipital Ctx | 0.0 |
| Control 2 Occipital Ctx | 100.0 |
| Control 3 Occipital Ctx | 13.3 |
| Control 4 Occipital Ctx | 2.2 |
| Control (Path) 1 Occipital Ctx | 100.0 |
| Control (Path) 2 Occipital Ctx | 7.2 |
| Control (Path) 3 Occipital Ctx | 0.0 |
| Control (Path) 4 Occipital Ctx | 18.9 |
| Control 1 Parietal Ctx | 2.5 |
| Control 2 Parietal Ctx | 53.2 |
| Control 3 Parietal Ctx | 21.6 |
| Control (Path) 1 Parietal Ctx | 94.6 |
| Control (Path) 2 Parietal Ctx | 16.8 |
| Control (Path) 3 Parietal Ctx | 4.0 |
| Control (Path) 4 Parietal Ctx | 50.3 |

TABLE XC
General_screening_panel_v1.5

| | Rel. Exp. (%) |
|---|---|
| | Ag5207, Run |
| Tissue Name | 228757767 |
| Adipose | 9.9 |
| Melanoma* Hs688(A).T | 4.0 |
| Melanoma* Hs688(B).T | 12.1 |
| Melanoma* M14 | 4.1 |
| Melanoma* LOXIMVI | 0.7 |
| Melanoma* SK-MEL-5 | 1.8 |
| Squamous cell carcinoma SCC-4 | 0.7 |
| Testis Pool | 2.8 |
| Prostate ca.* (bone met) PC-3 | 6.3 |
| Prostate Pool | 4.1 |
| Placenta | 0.2 |
| Uterus Pool | 5.6 |
| Ovarian ca. OVCAR-3 | 0.2 |
| Ovarian ca. SK-OV-3 | 5.9 |
| Ovarian ca. OVCAR-4 | 1.2 |
| Ovarian ca. OVCAR-5 | 1.6 |
| Ovarian ca. IGROV-1 | 0.8 |
| Ovarian ca. OVCAR-8 | 1.7 |
| Ovary | 0.7 |
| Breast ca. MCF-7 | 0.3 |
| Breast ca. MDA-MB-231 | 3.8 |
| Breast ca. BT 549 | 1.3 |
| Breast ca. T47D | 0.0 |
| Breast ca. MDA-N | 0.2 |
| Breast Pool | 6.4 |
| Trachea | 1.0 |
| Lung | 1.5 |
| Fetal Lung | 1.2 |
| Lung ca. NCI-N417 | 0.7 |
| Lung ca. LX-1 | 0.6 |
| Lung ca. NCI-H146 | 0.5 |
| Lung ca. SHP-77 | 0.4 |
| Lung ca. A549 | 4.8 |
| Lung ca. NCI-H526 | 0.6 |
| Lung ca. NCI-H23 | 0.2 |
| Lung ca. NCI-H460 | 3.2 |
| Lung ca. HOP-62 | 4.3 |
| Lung ca. NCI-H522 | 2.0 |
| Liver | 0.1 |
| Fetal Liver | 0.4 |
| Liver ca. HepG2 | 0.4 |
| Kidney Pool | 14.4 |
| Fetal Kidney | 0.2 |
| Renal ca. 786-0 | 1.2 |
| Renal ca. A498 | 1.2 |
| Renal ca. ACHN | 1.6 |
| Renal ca. UO-31 | 1.5 |
| Renal ca. TK-10 | 2.9 |
| Bladder | 2.2 |
| Gastric ca. (liver met.) NCI-N87 | 23.2 |
| Gastric ca. KATO III | 17.4 |
| Colon ca. SW-948 | 0.4 |
| Colon ca. SW480 | 1.3 |
| Colon ca.* (SW480 met) SW620 | 0.1 |
| Colon ca. HT29 | 0.3 |
| Colon ca. HCT-116 | 0.2 |
| Colon ca. CaCo-2 | 1.6 |
| Colon cancer tissue | 1.3 |
| Colon ca. SW1116 | 0.0 |
| Colon ca. Colo-205 | 2.6 |
| Colon ca. SW-48 | 0.8 |
| Colon Pool | 11.3 |
| Small Intestine Pool | 4.2 |
| Stomach Pool | 2.9 |
| Bone Marrow Pool | 2.1 |
| Fetal Heart | 45.7 |
| Heart Pool | 38.2 |
| Lymph Node Pool | 11.3 |
| Fetal Skeletal Muscle | 19.3 |
| Skeletal Muscle Pool | 100.0 |
| Spleen Pool | 0.5 |
| Thymus Pool | 4.0 |
| CNS cancer (glic/astro) U87-MG | 11.0 |
| CNS cancer (glio/astro) U-118-MG | 24.0 |
| CNS cancer (neuro; met) SK-N-AS | 3.4 |
| CNS cancer (astro) SF-539 | 1.0 |
| CNS cancer (astro) SNB-75 | 1.4 |
| CNS cancer (glio) SNB-19 | 1.2 |
| CNS cancer (glio) SF-295 | 18.6 |
| Brain (Amygdala) Pool | 3.7 |
| Brain (cerebellum) | 4.6 |
| Brain (fetal) | 0.2 |
| Brain (Hippocampus) Pool | 3.1 |
| Cerebral Cortex Pool | 6.7 |
| Brain (Substantia nigra) Pool | 4.3 |
| Brain (Thalamus) Pool | 8.2 |
| Brain (whole) | 4.4 |
| Spinal Cord Pool | 1.2 |
| Adrenal Gland | 2.6 |
| Pituitary gland Pool | 1.5 |
| Salivary Gland | 0.4 |
| Thyroid (female) | 0.4 |
| Pancreatic ca. CAPAN2 | 2.8 |
| Pancreas Pool | 6.0 |

TABLE XD
Panel 4.1D

| | Rel. Exp. (%) |
|---|---|
| | Ag5207, Run |
| Tissue Name | 229739304 |
| Secondary Th1 act | 0.0 |
| Secondary Th2 act | 4.2 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 5.6 |
| Primary Tr1 act | 5.6 |
| Primary Th1 rest | 0.0 |
| Primary Th2 rest | 0.0 |
| Primary Tr1 rest | 0.0 |
| CD45RA CD4 lymphocyte act | 35.6 |
| CD45RO CD4 lymphocyte act | 0.0 |
| CD8 lymphocyte act | 7.8 |
| Secondary CD8 lymphocyte rest | 0.0 |
| Secondary CD8 lymphocyte act | 0.0 |
| CD4 lymphocyte none | 10.7 |
| 2ry Th1/Th2/Tr1_anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 6.0 |
| NK Cells IL-2 rest | 4.8 |
| Two Way MLR 3 day | 0.0 |
| Two Way MLR 5 day | 0.0 |
| Two Way MLR 7 day | 0.0 |
| PBMC rest | 7.0 |
| PBMC PWM | 0.0 |
| PBMC PHA-L | 0.0 |
| Ramos (B cell) none | 0.0 |
| Ramos (B cell) ionomycin | 0.0 |
| B lymphocytes PWM | 0.0 |
| B lymphocytes CD40L and IL-4 | 0.0 |
| EOL-1 dbcAMP | 0.0 |
| EOL-1 dbcAMP PMA/ionomycin | 0.0 |
| Dendritic cells none | 5.1 |
| Dendritic cells LPS | 0.0 |
| Dendritic cells anti-CD40 | 0.0 |
| Monocytes rest | 6.6 |
| Monocytes LPS | 0.0 |
| Macrophages rest | 0.0 |
| Macrophages LPS | 0.0 |
| HUVEC none | 6.0 |
| HUVEC starved | 29.5 |
| HUVEC IL-1beta | 16.0 |
| HUVEC IFN gamma | 9.6 |
| HUVEC TNF alpha + IFN gamma | 3.5 |
| HUVEC TNF alpha + IL4 | 0.0 |
| HUVEC IL-11 | 5.5 |
| Lung Microvascular EC none | 7.1 |
| Lung Microvascular EC TNFalpha + | 0.0 |
| IL-1beta | |
| Microvascular Dermal EC none | 0.0 |
| Microsvasular Dermal EC TNFalpha + | 0.0 |
| IL-1beta | |
| Bronchial epithelium TNFalpha + | 0.0 |
| IL1beta | |
| Small airway epithelium none | 5.8 |
| Small airway epithelium TNFalpha | 3.6 |
| + IL-1beta | |
| Coronery artery SMC rest | 7.4 |
| Coronery artery SMC TNFalpha + | 13.6 |
| IL-1beta | |
| Astrocytes rest | 0.0 |
| Astrocytes TNFalpha + IL-1beta | 12.9 |
| KU-812 (Basophil) rest | 0.0 |
| KU-812 (Basophil) PMA/ionomycin | 0.0 |
| CCD1106 (Keratinocytes) none | 18.6 |
| CCD1106 (Keratinocytes) TNFalpha + | 0.0 |
| IL-1beta | |
| Liver cirrhosis | 0.0 |
| NCI-H292 none | 0.0 |
| NCI-H292 IL-4 | 0.0 |
| NCI-H292 IL-9 | 0.0 |
| NCI-H292 IL-13 | 0.0 |
| NCI-H292 IFN gamma | 0.0 |
| HPAEC none | 4.7 |
| HPAEC TNF alpha + IL-1 beta | 20.9 |
| Lung fibroblast none | 17.7 |
| Lung fibroblast TNF alpha + IL-1 | 23.0 |
| beta | |
| Lung fibroblast IL-4 | 10.8 |
| Lung fibroblast IL-9 | 11.3 |
| Lung fibroblast IL-13 | 9.2 |
| Lung fibroblast IFN gamma | 33.0 |
| Dermal fibroblast CCD1070 rest | 41.8 |
| Dermal fibroblast CCD1070 TNF alpha | 100.0 |
| Dermal fibroblast CCD1070 IL-1 beta | 77.9 |
| Dermal fibroblast IFN gamma | 7.6 |
| Dermal fibroblast IL-4 | 15.3 |
| Dermal Fibroblasts rest | 34.6 |
| Neutrophils TNFa + LPS | 4.8 |
| Neutrophils rest | 0.0 |
| Colon | 0.0 |
| Lung | 12.3 |
| Thymus | 0.0 |
| Kidney | 0.0 |

TABLE XE
Panel 5 Islet

| | Rel. Exp. (%) |
|---|---|
| | Ag5207, Run |
| Tissue Name | 263594763 |
| 97457_Patient-02go_adipose | 2.0 |
| 97476_Patient-07sk_skeletal muscle | 3.1 |
| 97477_Patient-07ut_uterus | 3.2 |
| 97478_Patient-07pl_placenta | 2.0 |
| 99167_Bayer Patient 1 | 1.0 |
| 97482_Patient-08ut_uterus | 6.7 |
| 97483_Patient-08pl_placenta | 0.0 |
| 97486_Patient-09sk_skeletal muscle | 27.4 |
| 97487_Patient-09ut_uterus | 12.4 |
| 97488_Patient-09pl_placenta | 1.3 |
| 97492_Patient-10ut_uterus | 14.4 |
| 97493_Patient-10pl_placenta | 2.1 |
| 97495_Patient-11go_adipose | 2.0 |
| 97496_Patient-11sk_skeletal muscle | 50.3 |
| 97497_Patient-11ut_uterus | 7.1 |
| 97498_Patient-11pl_placenta | 0.0 |
| 97500_Patient-12go_adipose | 10.7 |
| 97501_Patient-12sk_skeletal muscle | 100.0 |
| 97502_Patient-12ut_uterus | 10.9 |
| 97503_Patient-12pl_placenta | 0.0 |
| 94721_Donor 2 U - A_Mesenchymal Stem Cells | 1.8 |
| 94722_Donor 2 U - B_Mesenchymal Stem Cells | 1.0 |
| 94723_Donor 2 U - C_Mesenchymal Stem Cells | 3.5 |
| 94709_Donor 2 AM - A_adipose | 4.6 |
| 94710_Donor 2 AM - B_adipose | 1.1 |
| 94711_Donor 2 AM - C_adipose | 0.8 |
| 94712_Donor 2 AD - A_adipose | 1.0 |
| 94713_Donor 2 AD - B_adipose | 8.1 |
| 94714_Donor 2 AD - C_adipose | 5.3 |
| 94742_Donor 3 U - A_Mesenchymal Stem Cells | 1.2 |
| 94743_Donor 3 U - B_Mesenchymal Stem Cells | 3.7 |
| 94730_Donor 3 AM - A_adipose | 4.6 |
| 94731_Donor 3 AM - B_adipose | 2.1 |
| 94732_Donor 3 AM - C_adipose | 1.0 |
| 94733_Donor 3 AD - A_adipose | 6.9 |
| 94734_Donor 3 AD - B_adipose | 3.2 |
| 94735_Donor 3 AD - C_adipose | 4.4 |
| 77138_Liver_HepG2untreated | 3.4 |
| 73556_Heart_Cardiac stromal cells (primary) | 2.2 |
| 81735_Small Intestine | 7.1 |
| 72409_Kidney_Proximal Convoluted Tubule | 0.0 |
| 82685_Small intestine_Duodenum | 0.0 |
| 90650_Adrenal_Adrenocortical adenoma | 0.0 |
| 72410_Kidney_HRCE | 4.9 |
| 72411_Kidney_HRE | 0.0 |
| 73139_Uterus_Uterine smooth muscle cells | 4.0 |

CNS_neurodegeneration_v1.0 Summary: Ag5207 This panel does not show differential expression of this gene in Alzheimer's disease. However, this profile confirms the expression of this gene at moderate levels in the brain. Please see Panel 1.5 for discussion of this gene in the central nervous system.

General_screenin_panel_v1.5 Summary: Ag5207 Highest expression of this gene is seen in skeletal muscle (CT=28). Low but significant expression is also seen in pancreas, adrenal, pituitary, adipose, adult and fetal heart, and fetal skeletal muscle. This gene encodes a protein that is homologous to Glutamine:fructose-6-phosphate amidotransferase (GFAT) which catalyzes the formation of glucosamine 6-phosphate and is the first and rate-limiting enzyme of the hexosamine biosynthetic pathway. Enhanced glucose flux via the hexosamine biosynthetic pathway has been implicated in in the induction of insulin resistance. Buse et al. showed in a mouse model that glucose flux via the hexosariine pathway is selectively increased in muscle and may contribute to muscle insulin resistance in non-insulin-dependent diabetes mellitus. (Am J Physiol 1997 Jun;272(6 Pt 1):E1080-8). Thus, based on the homology of this enzyme to GFAT and the high expression in muscle, modulation of the expression or function of this gene may be useful in the treatment of type II diabetes.

This gene is widely expressed on this panel with moderate to low expression seen throughout the CNS, including the hippocampus, thalamus, substantia nigra, amygdala, cerebellum and cerebral cortex. Therefore, therapeutic modulation of the expression or function of this gene may be useful in the treatment of neurological disorders, such as Alzheimer's disease, Parkinson's disease, schizophrenia, multiple sclerosis, stroke and epilepsy.

Moderate to low levels of expression are also seen in many cancer cell lines on this panel, including gastric cancer and melanoma cell lines. Thus, modulation of this gene product may be useful in the treatment of cancer.

Panel 4.1D Summary: Ag5207 Detectable levels of expression appear to be restricted to TNF-alpha treated dermal fibroblasts (CT=33.3). This expression suggests that this gene product may be involved in skin disorders, including psoriasis.

Panel 5 Islet Summary: Ag5207 Highest expression is seen in skeletal muscle (CT=30.2), in agreement with panel 1.5. Moderate to low levels of expression are also seen in other metabolic tissues, including uterus and adipose. Please see Panel 1.5 for discussion of this gene in metabolic disease.

Y. CG148102-01: Carnitine O-Palmitoyltransferase I.

Expression of gene CG148102-01 was assessed using the primer-probe set Ag5274, described in Table YA. Results of the RTQ-PCR runs are shown in Tables YB, YC, YD and YE.

TABLE YA

Probe Name Ag5274

| | | | Start | SEQ ID |
|---|---|---|---|---|
| Primers | Sequence | Length | Position | No |
| Forward | 5′-cacttccgggacccacagt-3′ | 19 | 1732 | 339 |
| Probe | TET-5′-caccaggctctgctgaaggcagcc-3′-TAMRA | 24 | 1783 | 340 |
| Reverse | 5′-caaacaggtggcggtcaact-3′ | 20 | 1821 | 341 |

TABLE YB
CNS_neurodegeneration_v1.0

| | Rel. Exp. (%) |
|---|---|
| | Ag5274, Run |
| Tissue Name | 230512893 |
| AD 1 Hippo | 19.3 |
| AD 2 Hippo | 33.2 |
| AD 3 Hippo | 11.7 |
| AD 4 Hippo | 9.9 |
| AD 5 Hippo | 95.9 |
| AD 6 Hippo | 43.5 |
| Control 2 Hippo | 57.0 |
| Control 4 Hippo | 11.9 |
| Control (Path) 3 Hippo | 8.5 |
| AD 1 Temporal Ctx | 17.0 |
| AD 2 Temporal Ctx | 29.5 |
| AD 3 Temporal Ctx | 8.3 |
| AD 4 Temporal Ctx | 19.6 |
| AD 5 Inf Temporal Ctx | 95.9 |
| AD 5 Sup Temporal Ctx | 53.6 |
| AD 6 Inf Temporal Ctx | 29.9 |
| AD 6 Sup Temporal Ctx | 33.2 |
| Control 1 Temporal Ctx | 8.4 |
| Control 2 Temporal Ctx | 70.2 |
| Control 3 Temporal Ctx | 25.0 |
| Control 3 Temporal Ctx | 11.3 |
| Control (Path) 1 Temporal Ctx | 74.2 |
| Control (Path) 2 Temporal Ctx | 44.4 |
| Control (Path) 3 Temporal Ctx | 7.7 |
| Control (Path) 4 Temporal Ctx | 29.7 |
| AD 1 Occipital Ctx | 18.3 |
| AD 2 Occipital Ctx (Missing) | 0.0 |
| AD 3 Occipital Ctx | 7.5 |
| AD 4 Occipital Ctx | 15.1 |
| AD 5 Occipital Ctx | 66.4 |
| AD 6 Occipital Ctx | 13.1 |
| Control 1 Occipital Ctx | 3.7 |
| Control 2 Occipital Ctx | 98.6 |
| Control 3 Occipital Ctx | 27.5 |
| Control 4 Occipital Ctx | 4.5 |
| Control (Path) 1 Occipital Ctx | 100.0 |
| Control (Path) 2 Occipital Ctx | 17.1 |
| Control (Path) 3 Occipital Ctx | 3.8 |
| Control (Path) 4 Occipital Ctx | 20.0 |
| Control 1 Parietal Ctx | 10.5 |
| Control 2 Parietal Ctx | 49.3 |
| Control 3 Parietal Ctx | 19.2 |
| Control (Path) 1 Parietal Ctx | 94.6 |
| Control (Path) 2 Parietal Ctx | 25.0 |
| Control (Path) 3 Parietal Ctx | 6.0 |
| Control (Path) 4 Parietal Ctx | 50.7 |

YC
General_screening_panel_v1.5

| | Rel. Exp. (%) |
|---|---|
| | Ag5274, Run |
| Tissue Name | 230762793 |
| Adipose | 1.2 |
| Melanoma* Hs688(A).T | 7.4 |
| Melanoma* Hs688(B).T | 13.0 |
| Melanoma* M14 | 0.1 |
| Melanoma* LOXMVI | 0.0 |
| Melanoma* SK-MEL-5 | 0.0 |
| Squamous cell carcinoma SCC-4 | 1.5 |
| Testis Pool | 2.1 |
| Prostate ca.* (bone met) PC-3 | 21.8 |
| Prostate Pool | 0.8 |
| Placenta | 0.7 |
| Uterus Pool | 0.7 |
| Ovarian ca. OVCAR-3 | 12.2 |
| Ovarian ca. SK-OV-3 | 0.2 |
| Ovarian ca. OVCAR-4 | 0.1 |
| Ovarian ca. OVCAR-5 | 2.8 |
| Ovarian ca. IGROV-1 | 7.2 |
| Ovarian ca. OVCAR-8 | 3.9 |
| Ovary | 6.3 |
| Breast ca. MCF-7 | 0.2 |
| Breast ca. MDA-MB-231 | 4.9 |
| Breast ca. BT 549 | 88.3 |
| Breast ca. T47D | 0.0 |
| Breast ca. MDA-N | 0.0 |
| Breast Pool | 4.9 |
| Trachea | 1.0 |
| Lung | 0.9 |
| Fetal Lung | 7.2 |
| Lung ca. NCI-N417 | 8.2 |
| Lungca. LX-1 | 0.5 |
| Lung ca. NCI-H146 | 16.2 |
| Lung ca. SHP-77 | 53.6 |
| Lung ca. A549 | 0.0 |
| Lung ca. NCI-H526 | 3.6 |
| Lung ca. NCI-H23 | 40.9 |
| Lung ca. NCI-H460 | 0.6 |
| Lung ca. HOP-62 | 1.6 |
| Lung ca. NCI-H522 | 57.8 |
| Liver | 0.3 |
| Fetal Liver | 0.9 |
| Liver ca. HepG2 | 0.0 |
| Kidney Pool | 4.2 |
| Fetal Kidney | 3.6 |
| Renal ca. 786-0 | 0.0 |
| Renal ca. A498 | 0.0 |
| Renal ca. ACHN | 0.5 |
| Renal ca. UO-31 | 0.3 |
| Renal ca. TK-10 | 0.0 |
| Bladder | 1.7 |
| Gastric ca. (liver met.) NCI-N87 | 1.0 |
| Gastric ca. KATO III | 0.2 |
| Colon ca. SW-948 | 1.4 |
| Colon ca. SW480 | 0.7 |
| Colon ca.* (SW480 met) SW620 | 0.0 |
| Colon ca. HT29 | 0.2 |
| Colon ca. HCT-116 | 2.1 |
| Colon ca. CaCo-2 | 0.3 |
| Colon cancer tissue | 2.4 |
| Colon ca. SW1116 | 0.0 |
| Colon ca. Colo-205 | 0.0 |
| Colon ca. SW-48 | 0.0 |
| Colon Pool | 3.5 |
| Small Intestine Pool | 2.1 |
| Stomach Pool | 1.8 |
| Bone Marrow Pool | 0.8 |
| Fetal Heart | 1.7 |
| Heart Pool | 1.5 |
| Lymph Node Pool | 5.3 |
| Fetal Skeletal Muscle | 1.0 |
| Skeletal Muscle Pool | 0.8 |
| Spleen Pool | 3.0 |
| Thymus Pool | 2.7 |
| CNS cancer (glio/astro) U87-MG | 27.7 |
| CNS cancer (glio/astro) U-118-MG | 27.4 |
| CNS cancer (neuro; met) SK-N-AS | 86.5 |
| CNS cancer (astro) SF-539 | 0.0 |
| CNS cancer (astro) SNB-75 | 0.5 |
| CNS cancer (glio) SNB-19 | 7.2 |
| CNS cancer (glio) SF-295 | 17.3 |
| Brain (Amygdala) Pool | 19.9 |
| Brain (cerebellum) | 100.0 |
| Brain (fetal) | 44.8 |
| Brain (Hippocampus) Pool | 16.8 |
| Cerebral Cortex Pool | 24.0 |
| Brain (Substantia nigra) Pool | 27.4 |
| Brain (Thalamus) Pool | 34.2 |
| Brain (whole) | 42.0 |
| Spinal Cord Pool | 10.5 |
| Adrenal Gland | 1.0 |
| Pituitary gland Pool | 4.9 |
| Salivary Gland | 0.1 |
| Thyroid (female) | 0.6 |
| Pancreatic ca. CAPAN2 | 0.0 |
| Pancreas Pool | 4.8 |

TABLE YD
Panel 4.1D

| | Rel. Exp. (%) |
|---|---|
| | Ag5274, Run |
| Tissue Name | 230472159 |
| Secondary Th1 act | 2.3 |
| Secondary Th2 act | 1.6 |
| Secondary Tr1 act | 0.0 |
| Secondary Th1 rest | 0.0 |
| Secondary Th2 rest | 2.3 |
| Secondary Tr1 rest | 0.0 |
| Primary Th1 act | 0.0 |
| Primary Th2 act | 0.0 |
| Primary Tr1 act | 0.0 |
| Primary Th1 rest | 0.0 |
| Primary Th2 rest | 4.6 |
| Primary Tr1 rest | 0.0 |
| CD45RA CD4 lymphocyte act | 7.8 |
| CD45RO CD4 lymphocyte act | 0.0 |
| CD8 lymphocyte act | 0.0 |
| Secondary CD8 lymphocyte rest | 0.0 |
| Secondary CD8 lymphocyte act | 0.0 |
| CD4 lymphocyte none | 0.0 |
| 2ry Th1/Th2/Tr1_anti-CD95 CH11 | 0.0 |
| LAK cells rest | 0.0 |
| LAK cells IL-2 | 0.0 |
| LAK cells IL-2 + IL-12 | 0.0 |
| LAK cells IL-2 + IFN gamma | 0.0 |
| LAK cells IL-2 + IL-18 | 0.0 |
| LAK cells PMA/ionomycin | 0.0 |
| NK Cells IL-2 rest | 2.5 |
| Two Way MLR 3 day | 0.0 |
| Two Way MLR 5 day | 0.0 |
| Two Way MLR 7 day | 0.0 |
| PBMC rest | 0.0 |
| PBMC PWM | 2.2 |
| PBMC PHA-L | 10.1 |
| Ramos (B cell) none | 0.0 |
| Ramos (B cell) ionomycin | 0.0 |
| B lymphocytes PWM | 0.0 |
| B lymphocytes CD40L and IL-4 | 2.2 |
| EOL-1 dbcAMP | 9.2 |
| EOL-1 dbcAMP PMA/ionomycin | 2.7 |
| Dendritic cells none | 0.0 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 48.3 |
| HUVEC starved | 61.1 |
| HUVEC IL-1beta | 45.1 |
| HUVEC IFN gamma | 92.0 |
| HUVEC TNF alpha + IFN gamma | 15.1 |
| HUVEC TNF alpha + IL4 | 11.7 |
| HUVEC IL-11 | 67.8 |
| Lung Microvascular EC none | 38.2 |
| Lung Microvascular EC TNFalpha + IL-1beta | 9.2 |
| Microvascular Dermal EC none | 26.2 |
| Microsvasular Dermal EC TNFalpha + | 9.0 |
| IL-1beta | |
| Bronchial epithelium TNFalpha + IL1beta | 0.0 |
| Small airway epithelium none | 0.0 |
| Small airway epithelium TNFalpha + | 0.0 |
| IL-1beta | |
| Coronery artery SMC rest | 56.6 |
| Coronery artery SMC TNFalpha + IL-1beta | 66.9 |
| Astrocytes rest | 23.2 |
| Astrocytes TNFalpha + IL-1beta | 14.8 |
| KU-812 (Basophil) rest | 0.0 |
| KU-812 (Basophil) PMA/ionomycin | 0.0 |
| CCD1106 (Keratinocytes) none | 31.9 |
| CCD1106 (Keratinocytes) TNFalpha + | 9.4 |
| IL-1beta | |
| Liver cirrhosis | 5.1 |
| NCI-H292 none | 0.0 |
| NCI-H292 IL-4 | 0.0 |
| NCI-H292 IL-9 | 0.0 |
| NCI-H292 IL-13 | 0.0 |
| NCI-H292 IFN gamma | 8.6 |
| HPAEC none | 45.4 |
| HPAEC TNF alpha + IL-1 beta | 27.9 |
| Lung fibroblast none | 100.0 |
| Lung fibroblast TNF alpha + IL-1 beta | 90.8 |
| Lung fibroblast IL-4 | 22.2 |
| Lung fibroblast IL-9 | 47.6 |
| Lung fibroblast IL-13 | 11.8 |
| Lung fibroblast IFN gamma | 61.1 |
| Dermal fibroblast CCD1070 rest | 28.7 |
| Dermal fibroblast CCD1070 TNF alpha | 23.3 |
| Dermal fibroblast CCD1070 IL-1 beta | 28.7 |
| Dermal fibroblast IFN gamma | 16.7 |
| Dermal fibroblast IL-4 | 13.1 |
| Dermal Fibroblasts rest | 58.6 |
| Neutrophils TNFa + LPS | 0.0 |
| Neutrophils rest | 0.0 |
| Colon | 0.0 |
| Lung | 1.7 |
| Thymus | 0.0 |
| Kidney | 5.5 |

TABLE YE
Panel 5 Islet

| | Rel. Exp. (%) |
|---|---|
| | Ag5274, Run |
| Tissue Name | 307720339 |
| 97457_Patient-02go_adipose | 15.3 |
| 97476_Patient-07sk_skeletal muscle | 0.0 |
| 97477_Patient-07ut_uterus | 13.7 |
| 97478_Patient-07pl_placenta | 9.0 |
| 99167_Bayer Patient 1 | 51.8 |
| 97482_Patient-08ut_uterus | 24.3 |
| 97483_Patient-08pl_placenta | 0.0 |
| 97486_Patient-09sk_skeletal muscle | 0.0 |
| 97487_Patient-09ut_uterus | 7.3 |
| 97488_Patient-09pl_placenta | 11.9 |
| 97492_Patient-10ut_uterus | 12.8 |
| 97493_Patient-10pl_placenta | 5.3 |
| 97495_Patient-11go_adipose | 5.3 |
| 97496_Patient-11sk_skeletal muscle | 3.8 |
| 97497_Patient-11ut_uterus | 20.9 |
| 97498_Patient-11pl_placenta | 5.4 |
| 97500_Patient-12go_adipose | 27.0 |
| 9750l_Patient-12sk_skeletal muscle | 12.5 |
| 97502_Patient-12ut_uterus | 10.2 |
| 97503_Patient-12pl_placenta | 2.4 |
| 94721_Donor 2 U - A_Mesenchymal Stem | 100.0 |
| Cells | |
| 94722_Donor 2 U - B_Mesenchymal Stem | 43.2 |
| Cells | |
| 94723_Donor 2 U - C_Mesenchymal Stem | 63.7 |
| Cells | |
| 94709_Donor 2 AM - A_adipose | 13.9 |
| 94710_Donor 2 AM - B_adipose | 15.2 |
| 9471l_Donor 2 AM - C_adipose | 19.8 |
| 94712_Donor 2 AD - A_adipose | 58.2 |
| 94713_Donor 2 AD - B_adipose | 29.7 |
| 94714_Donor 2 AD - C_adipose | 34.9 |
| 94742_Donor 3 U - A_Mesenchymal Stem | 62.9 |
| Cells | |
| 94743_Donor 3 U - B_Mesenchymal Stem | 39.5 |
| Cells | |
| 94730_Donor 3 AM - A_adipose | 31.4 |
| 94731_Donor 3 AM - B_adipose | 35.1 |
| 94732_Donor 3 AM - C_adipose | 49.3 |
| 94733_Donor 3 AD - A_adipose | 28.9 |
| 94734_Donor 3 AD - B_adipose | 44.8 |
| 94735_Donor 3 AD - C_adipose | 17.7 |
| 77138_Liver_HepG2untreated | 6.0 |
| 73556_Heart_Cardiac stromal cells | 55.5 |
| (primary) | |
| 81735_Small Intestine | 39.0 |
| 72409_Kidney_Proximal Convoluted | 15.2 |
| Tubule | |
| 82685_Small intestine_Duodenum | 0.0 |
| 90650_Adrenal_Adrenocortical adenoma | 12.2 |
| 72410_Kidney_HRCE | 0.0 |
| 72411_Kidney_HRE | 25.7 |
| 73139_Uterus_Uterine smooth muscle | 97.9 |
| cells | |

CNS_neurodegeneration_v1.0 Summary: Ag5274 This panel confirms the expression of this gene at low levels in the brain in an independent group of individuals. This gene appears to be slightly down-regulated in the temporal cortex of Alzheimer's disease patients. Therefore, up-regulation of this gene or its protein product, or treatment with specific agonists for this receptor may be of use in reversing the dementia, memory loss, and neuronal death associated with this disease.

General_screen_panel_v1.5 Summary: Ag5274 Highest expression of this gene is seen in the cerebellum (CT=29.3). Moderate expression of this gene is seen throughout the brain. Thus, this gene would be useful for distinguishing brain tissue from non-neural tissue, and may be beneficial as a drug target in neurodegenerative disease, and specifically disorders that have this brain region as the site of pathology, such as autism and the ataxias. Please see Panel_CNS_neurodegeneration for further discussion of potential utility in the central nervous system.

Low but significant expression is also seen in pancreas. This gene encodes a protein with homology to carnitine palmitoyltransferase. Giannessi et al has shown that inhibition of this enzyme produces a significant reduction in serum glucose levels (J Med Chem 2001 Jul 19;44(15):2383-6). Thus, modulation of this enzyme may also be useful in the treatment of obesity and/or diabetes.

Panel 4.1D Summary: Ag5274 Highest expression of this gene is seen in untreated lung fibroblasts. Low, but significant expression is also seen in a cluster of treated and untreated lung and dermal fibroblasts. Low levels of expression are also seen in coronary artery SMCs, and HUVECs. This profile suggests that this gene could be used to differentiate between these cells and other cells samples. In addition, this gene product may be involved in inflammatory conditions of the lung and skin.

Panel 5 Islet Summary: Ag5274 Expression is limited to a sample derived from mesenchymal stem cells (CTs=34.5).

Z. CG148431-01 and CG148431-02: Aminotransferase Simolar to Serine Palmotyltransferase.

Expression of gene CG148431-01 and CG148431-02 was assessed using the primer-probe set Ag5627, described in Table ZA. Results of the RTQ-PCR runs are shown in Tables ZB, ZC, ZD and ZE. Please note that CG148431-02 represents a full-length physical clone of the CG148431-01 gene, validating the prediction of the gene sequence.

TABLE ZA

Probe Name Ag5627

| | | | | |
|---|---|---|---|---|
| | | | Start | SEQ ID |
| Primers | Sequences | Length | Position | No |

| | | | | |
|---|---|---|---|---|
| Forward | 5′-gggctcctataacttccttggt-3′ | 22 | 555 | 342 |
| Probe | TET-5′-tcctcatagactcatcatacttggctgca-3′-TAMRA | 39 | 579 | 343 |
| Reverse | 5′-cctgtgccatacacctctaaaa-3′ | 22 | 620 | 344 |

TABLE ZB
CNS_neurodegeneration_v1.0

| | Rel. Exp. (%) | Rel. Exp. (%) |
|---|---|---|
| | Ag5627, Run | Ag5627, Run |
| Tissue Name | 246956910 | 264979289 |
| AD 1 Hippo | 17.4 | 57.0 |
| AD 2 Hippo | 67.8 | 4.8 |
| AD 3 Hippo | 50.0 | 62.4 |
| AD 4 Hippo | 19.1 | 30.8 |
| AD 5 Hippo | 17.0 | 31.2 |
| AD 6 Hippo | 100.0 | 86.5 |
| Control 2 Hippo | 24.1 | 31.6 |
| Control 4 Hippo | 50.7 | 70.7 |
| Control (Path) 3 Hippo | 21.0 | 24.3 |
| AD 1 Temporal Ctx | 43.8 | 65.5 |
| AD 2 Temporal Ctx | 47.6 | 100.0 |
| AD 3 Temporal Ctx | 11.0 | 23.0 |
| AD 4 Temporal Ctx | 20.4 | 33.9 |
| AD 5 Inf Temporal Ctx | 31.0 | 31.2 |
| AD 5 Sup Temporal Ctx | 51.1 | 63.3 |
| AD 6 Inf Temporal Ctx | 68.8 | 87.7 |
| AD 6 Sup Temporal Ctx | 56.3 | 97.3 |
| Control 1 Temporal Ctx | 7.3 | 4.5 |
| Control 2 Temporal Ctx | 12.9 | 31.6 |
| Control 3 Temporal Ctx | 7.9 | 15.0 |
| Control 3 Temporal Ctx | 13.8 | 15.6 |
| Control (Path) 1 Temporal Ctx | 30.1 | 46.0 |
| Control (Path) 2 Temporal Ctx | 28.7 | 39.5 |
| Control (Path) 3 Temporal Ctx | 6.4 | 8.2 |
| Control (Path) 4 Temporal Ctx | 10.3 | 24.0 |
| AD 1 Occipital Ctx | 11.8 | 26.8 |
| AD 2 Occipital Ctx (Missing) | 0.0 | 0.0 |
| AD 3 Occipital Ctx | 4.2 | 25.9 |
| AD 4 Occipital Ctx | 20.0 | 27.9 |
| AD 5 Occipital Ctx | 37.4 | 17.0 |
| AD 6 Occipital Ctx | 29.1 | 22.4 |
| Control 1 Occipital Ctx | 3.9 | 12.1 |
| Control 2 Occipital Ctx | 20.6 | 29.9 |
| Control 3 Occipital Ctx | 9.3 | 19.9 |
| Control 4 Occipital Ctx | 16.3 | 44.1 |
| Control (Path) 1 Occipital Ctx | 49.0 | 58.2 |
| Control (Path) 2 Occipital Ctx | 6.6 | 15.2 |
| Control (Path) 3 Occipital Ctx | 0.0 | 1.6 |
| Control (Path) 4 Occipital Ctx | 23.3 | 14.3 |
| Control 1 Parietal Ctx | 13.1 | 18.3 |
| Control 2 Parietal Ctx | 31.6 | 68.8 |
| Control 3 Parietal Ctx | 7.9 | 19.8 |
| Control (Path) 1 Parietal Ctx | 63.7 | 87.1 |
| Control (Path) 2 Parietal Ctx | 51.1 | 57.4 |
| Control (Path) 3 Parietal Ctx | 3.1 | 6.1 |
| Control (Path) 4 Parietal Ctx | 54.7 | 59.5 |

TABLE ZC
Panel 4.1D

| | Rel. Exp. (%) |
|---|---|
| | Ag5627, Run |
| Tissue Name | 246490777 |
| Secondary Th1 act | 0.0 |
| Secondary Th2 act | 0.4 |
| Secondary Tr1 act | 0.0 |
| Secondary Th1 rest | 0.0 |
| Secondary Th2 rest | 0.0 |
| Secondary Tr1 rest | 0.0 |
| Primary Th1 act | 0.0 |
| Primary Th2 act | 0.2 |
| Primary Tr1 act | 0.2 |
| Primary Th1 rest | 0.0 |
| Primary Th2 rest | 0.0 |
| Primary Tr1 rest | 0.0 |
| CD45RA CD4 lymphocyte act | 2.7 |
| CD45RO CD4 lymphocyte act | 6.8 |
| CD8 lymphocyte act | 0.0 |
| Secondary CD8 lymphocyte rest | 0.8 |
| Secondary CD8 lymphocyte act | 0.0 |
| CD4 lymphocyte none | 0.0 |
| 2ry Th1/Th2/Tr1_anti-CD95 CH11 | 0.4 |
| LAK cells rest | 0.0 |
| LAK cells IL-2 | 0.0 |
| LAK cells IL-2 + IL-12 | 0.2 |
| LAK cells IL-2 + IFN gamma | 0.0 |
| LAK cells IL-2 + IL-18 | 0.0 |
| LAK cells PMA/ionomycin | 0.2 |
| NK Cells IL-2 rest | 11.8 |
| Two Way MLR 3 day | 0.4 |
| Two Way MLR 5 day | 0.0 |
| Two Way MLR 7 day | 0.0 |
| PBMC rest | 0.0 |
| PBMC PWM | 0.0 |
| PBMC PHA-L | 1.3 |
| Ramos (B cell) none | 0.0 |
| Ramos (B cell) ionomycin | 0.0 |
| B lymphocytes PWM | 0.0 |
| B lymphocytes CD40L and IL-4 | 0.0 |
| EOL-1 dbcAMP | 3.5 |
| EOL-1 dbcAMP PMA/ionomycin | 0.0 |
| Dendritic cells none | 1.1 |
| Dendritic cells LPS | 0.0 |
| Dendritic cells anti-CD40 | 0.0 |
| Monocytes rest | 0.0 |
| Monocytes LPS | 0.0 |
| Macrophages rest | 0.0 |
| Macrophages LPS | 0.0 |
| HUVEC none | 0.7 |
| HUVEC starved | 2.9 |
| HUVEC IL-1beta | 0.0 |
| HUVEC IFN gamma | 16.7 |
| HUVEC TNF alpha + IFN gamma | 0.3 |
| HUVEC TNF alpha + IL4 | 0.0 |
| HUVEC IL-11 | 1.2 |
| Lung Microvascular EC none | 0.4 |
| Lung Microvascular EC TNFalpha + IL-1beta | 0.0 |
| Microvascular Dermal EC none | 0.0 |
| Microsvasular Dermal EC TNFalpha + IL- | 0.0 |
| 1beta | |
| Bronchial epithelium TNFalpha + IL1beta | 8.4 |
| Small airway epithelium none | 18.7 |
| Small airway epithelium TNFalpha + IL- | 24.3 |
| 1beta | |
| Coronery artery SMC rest | 3.3 |
| Coronery artery SMC TNFalpha + IL-1beta | 2.8 |
| Astrocytes rest | 3.9 |
| Astrocytes TNFalpha + IL-1beta | 1.4 |
| KU-812 (Basophil) rest | 8.0 |
| KU-812 (Basophil) PMA/ionomycin | 14.2 |
| CCD1106 (Keratinocytes) none | 17.4 |
| CCD1106 (Keratinocytes) TNFalpha + IL- | 24.3 |
| 1beta | |
| Liver cirrhosis | 13.3 |
| NCI-H292 none | 10.2 |
| NCI-H292 IL-4 | 36.3 |
| NCI-H292 IL-9 | 21.5 |
| NCI-H292 IL-13 | 27.7 |
| NCI-H292 IFN gamma | 18.3 |
| HPAEC none | 0.8 |
| HPAEC TNFalpha + IL-1 beta | 0.3 |
| Lung fibroblast none | 21.5 |
| Lung fibroblast TNF alpha + IL-1 beta | 2.7 |
| Lung fibroblast IL-4 | 10.2 |
| Lung fibroblast IL-9 | 6.2 |
| Lung fibroblast IL-13 | 1.3 |
| Lung fibroblast IFN gamma | 43.5 |
| Dermal fibroblast CCD1070 rest | 0.0 |
| Dermal fibroblast CCD1070 TNF alpha | 1.1 |
| Dermal fibroblast CCD1070 IL-1 beta | 1.6 |
| Dermal fibroblast IFN gamma | 39.5 |
| Dermal fibroblast IL-4 | 12.0 |
| Dermal Fibroblasts rest | 16.0 |
| Neutrophils TNFa + LPS | 0.0 |
| Neutrophils rest | 0.0 |
| Colon | 3.0 |
| Lung | 4.6 |
| Thymus | 3.5 |
| Kidney | 100.0 |

TABLE ZD
Panel 5 Islet

| | Rel. Exp. (%) | Rel. Exp. (%) |
|---|---|---|
| | Ag5627, Run | Ag5627, Run |
| Tissue Name | 279371483 | 312852505 |
| 97457_Patient-02go_adipose | 0.7 | 1.7 |
| 97476_Patient-07sk_skeletal | 0.0 | 0.0 |
| muscle | | |
| 97477_Patient-07ut_uterus | 0.4 | 0.5 |
| 97478_Patient-07pl_placenta | 40.3 | 46.0 |
| 99167_Bayer Patient 1 | 0.1 | 0.1 |
| 97482_Patient-08ut_uterus | 0.2 | 0.2 |
| 97483_Patient-08pl_placenta | 82.9 | 100.0 |
| 97486_Patient-09sk_skeletal | 0.2 | 0.1 |
| muscle | | |
| 97487_Patient-09ut_uterus | 0.2 | 0.5 |
| 97488_Patient-09pl_placenta | 29.9 | 25.5 |
| 97492_Patient-10ut_uterus | 0.3 | 0.4 |
| 97493_Patient-10pl_placenta | 100.0 | 71.7 |
| 97495_Patient-11go_adipose | 1.2 | 0.9 |
| 97496_Patient-11sk_skeletal | 0.2 | 0.1 |
| muscle | | |
| 97497_Patient-11ut_uterus | 0.5 | 0.8 |
| 97498_Patient-11pl_placenta | 28.1 | 31.6 |
| 97500_Patient-12go_adipose | 1.0 | 1.8 |
| 97501_Patient-12sk_skeletal | 0.5 | 0.6 |
| muscle | | |
| 97502_Patient-12ut_uterus | 0.3 | 0.4 |
| 97503_Patient-12pl_placenta | 85.9 | 88.3 |
| 94721_Donor 2 U - A_{—} | 1.2 | 1.3 |
| Mesenchymal Stem Cells | | |
| 94722_Donor 2 U - B_{—} | 0.6 | 0.8 |
| Mesenchymal Stem Cells | | |
| 94723_Donor 2 U - C_{—} | 1.0 | 1.3 |
| Mesenchymal Stem Cells | | |
| 94709_Donor 2 AM - A_adipose | 1.2 | 1.6 |
| 94710_Donor 2 AM - B_adipose | 1.1 | 1.7 |
| 94711_Donor 2 AM - C_adipose | 0.8 | 1.4 |
| 94712_Donor 2 AD - A_adipose | 2.7 | 2.0 |
| 94713_Donor 2 AD - B_adipose | 4.0 | 3.0 |
| 94714_Donor 2 AD - C_adipose | 3.0 | 3.0 |
| 94742_Donor 3 U - A_{—} | 0.4 | 0.4 |
| Mesenchymal Stem Cells | | |
| 94743_Donor 3 U - B_{—} | 0.3 | 0.6 |
| Mesenchymal Stem Cells | | |
| 94730_Donor 3 AM - A_adipose | 3.5 | 3.7 |
| 94731_Donor 3 AM - B_adipose | 5.3 | 5.6 |
| 94732_Donor 3 AM - C_adipose | 3.9 | 4.8 |
| 94733_Donor 3 AD - A_adipose | 2.6 | 3.5 |
| 94734_Donor 3 AD - B_adipose | 2.8 | 3.6 |
| 94735_Donor 3 AD - C_adipose | 0.5 | 0.8 |
| 77138_Liver_HepG2untreated | 39.5 | 43.2 |
| 73556_Heart_Cardiac stromal | 0.1 | 0.0 |
| cells (primary) | | |
| 81735_Small Intestine | 1.8 | 1.9 |
| 72409_Kidney_Proximal | 18.2 | 19.1 |
| Convoluted Tubule | | |
| 82685_Small intestine_{—} | 1.3 | 1.1 |
| Duodenum | | |
| 90650_Adrenal_{—} | 0.6 | 0.4 |
| Adrenocortical adenoma | | |
| 72410_Kidney_HRCE | 3.7 | 4.9 |
| 72411_Kidney_HRE | 1.6 | 1.7 |
| 73139_Uterus_Uterine | 1.0 | 0.7 |
| smooth muscle cells | | |

TABLE ZE
general oncology screening panel_v_2.4

| | Rel. Exp. (%) |
|---|---|
| | Ag5627, Run |
| Tissue Name | 268787222 |
| Colon cancer 1 | 2.8 |
| Colon NAT 1 | 2.7 |
| Colon cancer 2 | 7.8 |
| Colon NAT 2 | 3.1 |
| Colon cancer 3 | 5.7 |
| Colon NAT 3 | 6.4 |
| Colon malignant cancer 4 | 3.0 |
| Colon NAT 4 | 2.4 |
| Lung cancer 1 | 2.9 |
| Lung NAT 1 | 1.1 |
| Lung cancer 2 | 16.2 |
| Lung NAT 2 | 2.3 |
| Squamous cell carcinoma 3 | 4.8 |
| Lung NAT 3 | 0.5 |
| Metastatic melanoma 1 | 8.7 |
| Melanoma 2 | 3.7 |
| Melanoma 3 | 9.2 |
| Metastatic melanoma 4 | 16.3 |
| Metastatic melanoma 5 | 20.2 |
| Bladder cancer 1 | 1.3 |
| Bladder NAT 1 | 0.0 |
| Bladder cancer 2 | 3.9 |
| Bladder NAT 2 | 0.3 |
| Bladder NAT 3 | 0.2 |
| Bladder NAT 4 | 1.1 |
| Prostate adenocarcinoma 1 | 11.8 |
| Prostate adenocarcinoma 2 | 1.0 |
| Prostate adenocarcinoma 3 | 8.6 |
| Prostate adenocarcinoma 4 | 1.7 |
| Prostate NAT 5 | 1.1 |
| Prostate adenocarcinoma 6 | 2.6 |
| Prostate adenocarcinoma 7 | 3.3 |
| Prostate adenocarcinoma 8 | 0.6 |
| Prostate adenocarcinoma 9 | 6.5 |
| Prostate NAT 10 | 1.4 |
| Kidney cancer 1 | 14.2 |
| Kidney NAT 1 | 7.6 |
| Kidney cancer 2 | 100.0 |
| Kidney NAT 2 | 15.6 |
| Kidney cancer 3 | 38.7 |
| Kidney NAT 3 | 6.5 |
| Kidney cancer 4 | 11.8 |
| Kidney NAT 4 | 6.9 |

CNS_neurodegeneration_v1.0 Summary: Ag5627 Two experiments with same probe-primer sets are in good agreements. This panel confirms the expression of this gene at low levels in the brain in an independent group of individuals. This gene is found to be upregulated in the temporal cortex of Alzheimer's disease patients. Therefore, therapeutic modulation of the expression or function of this gene may decrease neuronal death and be of use in the treatment of this disease.

Panel 4.1D Summary: Ag5627 Highest expression of this gene is detected in kidney. Moderate to low levels of expression of this gene is also seen in activated naive and memory T cells, IL-2 treated NK cells, IFN gamma activated HUVEC cells, cytokine activated bronchial epithelial cells, astrocytes, resting and activated small airway epithelial cells, coronery artery SMC cells, basophils, keratinocytes, mucoepidermoid NCI-H292 cells, lung and dermal fibroblast, liver cirrhosis sample and normal tissues such as colon, lung, and thymus. Therefore, therapeutic modulation of this gene or its protein product through the use of small molecule drug may be useful in the treatment of autoimmune and inflammatory diseases such as asthma, allergies, inflammatory bowel disease, lupus erythematosus, psoriasis, rheumatoid arthritis, and osteoarthritis.

Panel 5 Islet Summary: Ag5627 Two experiments with same probe and primer sets are in good agreements. Highest expression of this gene is detected in placenta of diabetic and nondiabetic patients (CTs=26.4-26.7). Moderate to high levels of expression of this gene is also seen in liver HepG2 cell line, adipose, small intestine and kidney. This gene codes for a homolog of Serine palmitoyltransferase 2. Serine palmitoyltransferase catalyzes the first, rate limiting step in de novo ceramide biosynthesis. C2-ceramide inhibits GLUT4 translocation by inhibiting Akt phosphorylation and activation in 3T3-L1 adipocytes, independently of effects on IRS-1 (Summers et al., 1998, Mol Cell Biol 18:5457-64, PMID: 9710629). Ceramide downregulates PDE3B and induces lipolysis in 3T3-L1 cells. Ceramide effects are reversed by troglitazone (Mei et al., 2002, Diabetes 51: 631-7, PMID: 11872660). Palmitate-induced insulin resistance involves elevation of de novo ceramide synthesis in C2C12 myotubes (Schrmitz-Peiffer et al., 1999, J Biol Chem 274:24202, PMID: 10446195). Therefore, inhibition of the novel serine palmitoyltransferase through the use of small molecule drug may be beneficial in the treatment of diabetes.

General oncology screening panel_v_v2.4 Summary: Ag5627 Highest expression of this gene is detected in kidney cancer (CT=27.5). Moderate to high expression of this gene is also seen in normal and cancer samples derived from colon, lung, bladder, prostate and kidney. Moderate levels of expression of this gene is also seen in melanoma and metastatic melanoma samples. Expression of this gene is strongly associated with kidney, lung and bladder cancers as compared to the corresponding normal tissues. Therefore, expression of this gene may be used as diagnostic marker for detection of these cancers and also, therapeutic modulation of this gene or its protein product may be useful in the treatment of melanoma, colon, lung, bladder, prostate and kidney cancers.

AA. CG148888-01: Galnac 4-Sulfotransferase.

Expression of gene CG148888-01 was assessed using the primer-probe set Ag6854, described in Table AAA. Results of the RTQ-PCR runs are shown in Table AAB. Please note that CG148888-01 represents a full-length physical clone.

TABLE AAA

Probe Name Ag6854

| | | | Start | SEQ ID |
|---|---|---|---|---|
| Primers | Sequences | Length | Position | No |
| Forward | 5′-accccagagccgcctggt-3′ | 18 | 369 | 345 |
| Probe | TET-5′-cttggcctgatgttgaactttattcctggcacc-3′-TAMRA | 33 | 408 | 346 |
| Reverse | 5′-cagcctgcaggaccctacg-3′ | 19 | 458 | 347 |

TABLE AAB
General_screening_panel_v1.6

| | Rel. Exp. (%) |
|---|---|
| | Ag6854, Run |
| Tissue Name | 278020603 |
| Adipose | 0.0 |
| Melanoma* Hs688(A).T | 0.0 |
| Melanoma* Hs688(B).T | 0.2 |
| Melanoma* M14 | 0.0 |
| Melanoma* LOXIMVI | 0.0 |
| Melanoma* SK-MEL-5 | 0.3 |
| Squamous cell carcinoma SCC-4 | 0.1 |
| Testis Pool | 0.2 |
| Prostate ca.* (bone met) PC-3 | 0.0 |
| Prostate Pool | 0.0 |
| Placenta | 0.0 |
| Uterus Pool | 0.0 |
| Ovarian ca. OVCAR-3 | 0.0 |
| Ovarian ca. SK-OV-3 | 0.0 |
| Ovarian ca. OVCAR-4 | 0.0 |
| Ovarian ca. OVCAR-5 | 0.1 |
| Ovarian ca. IGROV-1 | 0.0 |
| Ovarian ca. OVCAR-8 | 0.0 |
| Ovary | 0.2 |
| Breast ca. MCF-7 | 0.7 |
| Breast ca. MDA-MB-231 | 0.0 |
| Breast ca. BT 549 | 0.0 |
| Breast ca. T47D | 0.0 |
| Breast ca. MDA-N | 0.0 |
| Breast Pool | 0.2 |
| Trachea | 0.3 |
| Lung | 0.2 |
| Fetal Lung | 0.0 |
| Lung ca. NCI-N417 | 0.0 |
| Lung ca. LX-1 | 0.0 |
| Lung ca. NCI-H146 | 0.0 |
| Lung ca. SHP-77 | 100.0 |
| Lung ca. A549 | 0.0 |
| Lung ca. NCI-H526 | 0.4 |
| Lung ca. NCI-H23 | 0.2 |
| Lung ca. NCI-H460 | 0.1 |
| Lung ca. HOP-62 | 0.0 |
| Lung ca. NCI-H522 | 1.4 |
| Liver | 0.0 |
| Fetal Liver | 0.0 |
| Liver ca. HepG2 | 0.0 |
| Kidney Pool | 0.0 |
| Fetal Kidney | 0.0 |
| Renal ca. 786-0 | 0.0 |
| Renal ca. A498 | 0.0 |
| Renal ca. ACHN | 0.0 |
| Renal ca. UO-31 | 0.0 |
| Renal ca. TK-10 | 0.0 |
| Bladder | 0.1 |
| Gastric ca. (liver met.) NCI-N87 | 0.0 |
| Gastric ca. KATO III | 0.0 |
| Colon ca. SW-948 | 0.0 |
| Colon ca. SW480 | 0.1 |
| Colon ca.* (SW480 met) SW620 | 0.0 |
| Colon ca. HT29 | 0.0 |
| Colon ca. HCT-116 | 0.0 |
| Colon ca. CaCo-2 | 0.0 |
| Colon cancer tissue | 0.1 |
| Colon ca. SW1116 | 0.0 |
| Colon ca. Colo-205 | 0.0 |
| Colon ca. SW-48 | 0.0 |
| Colon Pool | 0.2 |
| Small Intestine Pool | 0.1 |
| Stomach Pool | 0.3 |
| Bone Marrow Pool | 0.1 |
| Fetal Heart | 0.3 |
| Heart Pool | 0.0 |
| Lymph Node Pool | 0.5 |
| Fetal Skeletal Muscle | 0.0 |
| Skeletal Muscle Pool | 0.0 |
| Spleen Pool | 0.6 |
| Thymus Pool | 0.5 |
| CNS cancer (glio/astro) U87-MG | 0.0 |
| CNS cancer (glio/astro) U-118-MG | 0.0 |
| CNS cancer (neuro; met) SK-N-AS | 2.2 |
| CNS cancer (astro) SF-539 | 0.0 |
| CNS cancer (astro) SNB-75 | 0.7 |
| CNS cancer (glio) SNB-19 | 0.0 |
| CNS cancer (glio) SF-295 | 0.1 |
| Brain (Amygdala) Pool | 3.7 |
| Brain (cerebellum) | 8.8 |
| Brain (fetal) | 16.2 |
| Brain (Hippocampus) Pool | 3.6 |
| Cerebral Cortex Pool | 3.7 |
| Brain (Substantia nigra) Pool | 4.6 |
| Brain (Thalamus) Pool | 5.0 |
| Brain (whole) | 4.5 |
| Spinal Cord Pool | 4.7 |
| Adrenal Gland | 0.2 |
| Pituitary gland Pool | 8.0 |
| Salivary Gland | 0.0 |
| Thyroid (female) | 0.2 |
| Pancreatic ca. CAPAN2 | 0.1 |
| Pancreas Pool | 0.2 |

General_screen_panel_v1.6 Summary: Ag6854 Highest expression of this gene is seen in a lung cancer cell line (CT=27.8). Thus, expression of this gene could be used to differentiate between this sample and other samples on this panel and as a marker to detect the presence of lung cancer. Furthermore, therapeutic modulation of the expression or function of this gene may be effective in the treatment of lung cancer.

This gene is also expressed at moderate to low levels in the CNS, including the hippocampus, thalamus, substantia nigra, amygdala, cerebellum and cerebral cortex. Therefore, therapeutic modulation of the expression or function of this gene may be useful in the treatment of neurological disorders, such as Alzheimer's disease, Parkinson's disease, schizophrenia, multiple sclerosis, stroke and epilepsy.

AB. CGI49008-01: Novel Sodium/Hydrogen Exchanger Family Member.

Expression of gene CGI49008-01 was assessed using the primer-probe set Ag5630, described in Table ABA. Results of the RTQ-PCR runs are shown in Tables ABB, ABC, ABD and ABE.

TABLE ABA

Probe Name Ag5630

| | | | Start | SEQ ID |
|---|---|---|---|---|
| Primers | Sequences | Length | Position | No |
| Forward | 5′-tattttctgggtcaggctgat-3′ | 21 | 770 | 348 |
| Probe | TET-5′-tctctaaactcaacatgacagacagttttg-3′-TAMRA | 30 | 795 | 349 |
| Reverse | 5′-cagatattagggagccaaacg-3′ | 21 | 825 | 350 |

TABLE ABB
CNS_neurodegeneration_v1.0

| | Rel. Exp. (%) |
|---|---|
| | Ag5630, Run |
| Tissue Name | 246956911 |
| AD 1 Hippo | 9.3 |
| AD 2 Hippo | 31.4 |
| AD 3 Hippo | 5.5 |
| AD 4 Hippo | 8.4 |
| AD 5 hippo | 62.0 |
| AD 6 Hippo | 46.0 |
| Control 2 Hippo | 31.4 |
| Control 4 Hippo | 15.9 |
| Control (Path) 3 Hippo | 10.4 |
| AD 1 Temporal Ctx | 12.0 |
| AD 2 Temporal Ctx | 41.8 |
| AD 3 Temporal Ctx | 2.3 |
| AD 4 Temporal Ctx | 25.7 |
| AD 5 Inf Temporal Ctx | 100.0 |
| AD 5 Sup Temporal Ctx | 48.6 |
| AD 6 Inf Temporal Ctx | 36.9 |
| AD 6 Sup Temporal Ctx | 45.7 |
| Control 1 Temporal Ctx | 14.3 |
| Control 2 Temporal Ctx | 48.6 |
| Control 3 Temporal Ctx | 12.8 |
| Control 4 Temporal Ctx | 14.1 |
| Control (Path) 1 Temporal Ctx | 52.5 |
| Control (Path) 2 Temporal Ctx | 33.9 |
| Control (Path) 3 Temporal Ctx | 9.3 |
| Control (Path) 4 Temporal Ctx | 14.5 |
| AD 1 Occipital Ctx | 7.5 |
| AD 2 Occipital Ctx (Missing) | 0.0 |
| AD 3 Occipital Ctx | 4.5 |
| AD 4 Occipital Ctx | 18.9 |
| AD 5 Occipital Ctx | 13.9 |
| AD 6 Occipital Ctx | 46.3 |
| Control 1 Occipital Ctx | 3.8 |
| Control 2 Occipital Ctx | 61.6 |
| Control 3 Occipital Ctx | 6.1 |
| Control 4 Occipital Ctx | 13.2 |
| Control (Path) 1 Occipital Ctx | 62.0 |
| Control (Path) 2 Occipital Ctx | 10.5 |
| Control (Path) 3 Occipital Ctx | 8.4 |
| Control (Path) 4 Occipital Ctx | 11.8 |
| Control 1 Parietal Ctx | 10.4 |
| Control 2 Parietal Ctx | 49.0 |
| Control 3 Parietal Ctx | 20.3 |
| Control (Path) 1 Parietal Ctx | 44.1 |
| Control (Path) 2 Parietal Ctx | 22.7 |
| Control (Path) 3 Parietal Ctx | 8.2 |
| Control (Path) 4 Parietal Ctx | 35.1 |

TABLE ABC
General_screening_panel_v1.5

| | Rel. Exp. (%) |
|---|---|
| | Ag5630, Run |
| Tissue Name | 245065625 |
| Adipose | 4.2 |
| Melanoma* Hs688(A).T | 21.9 |
| Melanoma* Hs688(B).T | 19.2 |
| Melanoma* M14 | 41.2 |
| Melanoma* LOXIMVI | 25.2 |
| Melanoma* SK-MEL-5 | 20.0 |
| Squamous cell carcinoma SCC-4 | 8.4 |
| Testis Pool | 9.1 |
| Prostate ca.* (bone met) PC-3 | 5.8 |
| Prostate Pool | 3.0 |
| Placenta | 16.7 |
| Uterus Pool | 4.3 |
| Ovarian ca. OVCAR-3 | 35.6 |
| Ovarian ca. SK-OV-3 | 15.4 |
| Ovarian ca. OVCAR-4 | 9.5 |
| Ovarian ca. OVCAR-5 | 44.8 |
| Ovarian ca. IGROV-l | 13.9 |
| Ovarian ca. OVCAR-8 | 8.0 |
| Ovary | 3.8 |
| Breast ca. MCF-7 | 14.9 |
| Breast ca. MDA-MB-231 | 25.2 |
| Breast ca. BT 549 | 32.1 |
| Breast ca. T47D | 18.7 |
| Breast ca. MDA-N | 9.3 |
| Breast Pool | 1.7 |
| Trachea | 18.4 |
| Lung | 1.7 |
| Fetal Lung | 9.2 |
| Lung ca. NCI-N417 | 4.8 |
| Lung ca. LX-1 | 24.1 |
| Lung ca. NCI-H146 | 3.6 |
| Lung ca. SHP-77 | 14.0 |
| Lung ca. A549 | 35.4 |
| Lung ca. NCI-H526 | 3.5 |
| Lung ca. NCI-H23 | 23.5 |
| Lung ca. NCI-H460 | 6.7 |
| Lung ca. HOP-62 | 7.6 |
| Lung ca. NCI-H522 | 8.5 |
| Liver | 4.2 |
| Fetal Liver | 15.8 |
| Liver ca. HepG2 | 5.7 |
| Kidney Pool | 7.7 |
| Fetal Kidney | 5.0 |
| Renal ca. 786-0 | 19.9 |
| Renal ca. A498 | 14.3 |
| Renal ca. ACHN | 8.9 |
| Renal ca. UO-31 | 32.1 |
| Renal ca. TK-10 | 32.8 |
| Bladder | 9.5 |
| Gastric ca. (liver met.) NCI-N87 | 100.0 |
| Gastric ca. KATO III | 52.1 |
| Colon ca. SW-948 | 5.1 |
| Colon ca. SW480 | 27.2 |
| Colon ca.* (SW480 met) SW620 | 22.2 |
| Colon ca. HT29 | 10.5 |
| Colon ca. HCT-116 | 15.6 |
| Colon ca. CaCo-2 | 25.9 |
| Colon cancer tissue | 12.9 |
| Colon ca. SW1116 | 3.4 |
| Colon ca. Colo-205 | 19.8 |
| Colon ca. SW-48 | 12.6 |
| Colon Pool | 6.4 |
| Small Intestine Pool | 4.0 |
| Stomach Pool | 3.7 |
| Bone Marrow Pool | 2.9 |
| Fetal Heart | 4.1 |
| Heart Pool | 3.3 |
| Lymph Node Pool | 6.8 |
| Fetal Skeletal Muscle | 2.5 |
| Skeletal Muscle Pool | 15.6 |
| Spleen Pool | 5.4 |
| Thymus Pool | 7.6 |
| CNS cancer (glio/astro) U87-MG | 74.2 |
| CNS cancer (glio/astro) U-118-MG | 34.4 |
| CNS cancer (neuro; met) SK-N-AS | 8.5 |
| CNS cancer (astro) SF-539 | 11.9 |
| CNS cancer (astro) SNB-75 | 43.2 |
| CNS cancer (glio) SNB-19 | 12.9 |
| CNS cancer (glio) SF-295 | 30.8 |
| Brain (Amygdala) Pool | 4.9 |
| Brain (cerebellum) | 23.7 |
| Brain (fetal) | 6.5 |
| Brain (Hippocampus) Pool | 7.5 |
| Cerebral Cortex Pool | 5.3 |
| Brain (Substantia nigra) Pool | 4.3 |
| Brain (Thalamus) Pool | 7.4 |
| Brain (whole) | 5.4 |
| Spinal Cord Pool | 6.4 |
| Adrenal Gland | 24.1 |
| Pituitary gland Pool | 3.1 |
| Salivary Gland | 13.2 |
| Thyroid (female) | 8.1 |
| Pancreatic ca. CAPAN2 | 26.1 |
| Pancreas Pool | 9.3 |

TABLE ABD
Panel 4.1D

| | Rel. Exp. (%) |
|---|---|
| | Ag5630, Run |
| Tissue Name | 246490808 |
| Secondary Th1 act | 52.9 |
| Secondary Th2 act | 86.5 |
| Secondary Tr1 act | 14.5 |
| Secondary Th1 rest | 2.2 |
| Secondary Th2 rest | 1.7 |
| Secondary Tr1 rest | 0.0 |
| Primary Th1 act | 0.8 |
| Primary Th2 act | 42.6 |
| Primary Tr1 act | 35.4 |
| Primary Th1 rest | 1.9 |
| Primary Th2 rest | 3.4 |
| Primary Tr1 rest | 0.3 |
| CD45RA CD4 lymphocyte act | 30.6 |
| CD45RO CD4 lymphocyte act | 49.3 |
| CD8 lymphocyte act | 4.6 |
| Secondary CD8 lymphocyte rest | 29.9 |
| Secondary CD8 lymphocyte act | 6.6 |
| CD4 lymphocyte none | 0.0 |
| 2ry Th1/Th2/Tr1_anti-CD95 CH11 | 2.5 |
| LAK cells rest | 11.1 |
| LAK cells IL-2 | 9.7 |
| LAK cells IL-2 + IL-12 | 2.3 |
| LAK cells IL-2 + IFN gamma | 17.3 |
| LAK cells IL-2 + IL-18 | 9.5 |
| LAK cells PMA/ionomycin | 36.3 |
| NK Cells IL-2 rest | 17.0 |
| Two Way MLR 3 day | 9.4 |
| Two Way MLR 5 day | 1.0 |
| Two Way MLR 7 day | 7.0 |
| PBMC rest | 0.9 |
| PBMC PWM | 9.9 |
| PBMC PHA-L | 8.4 |
| Ramos (B cell) none | 1.4 |
| Ramos (B cell) ionomycin | 28.5 |
| B lymphocytes PWM | 19.6 |
| B lymphocytes CD40L and IL-4 | 28.1 |
| EOL-1 dbcAMP | 3.8 |
| EOL-1 dbcAMP PMA/ionomycin | 0.4 |
| Dendritic cells none | 9.2 |
| Dendritic cells LPS | 3.2 |
| Dendritic cells anti-CD40 | 3.8 |
| Monocytes rest | 0.0 |
| Monocytes LPS | 100.0 |
| Macrophages rest | 6.0 |
| Macrophages LPS | 10.6 |
| HUVEC none | 12.6 |
| HUVEC starved | 21.5 |
| HUVEC IL-1beta | 21.9 |
| HUVEC IFN gamma | 20.2 |
| HUVEC TNF alpha + IFN gamma | 6.7 |
| HUVEC TNF alpha + IL4 | 4.6 |
| HUVEC IL-11 | 12.6 |
| Lung Microvascular EC none | 31.6 |
| Lung Microvascular EC TNFalpha + IL- | 9.4 |
| 1beta | |
| Microvascular Dermal EC none | 0.7 |
| Microsvasular Dermal EC TNFalpha + IL- | 7.2 |
| 1beta | |
| Bronchial epithelium TNFalpha + IL1beta | 4.2 |
| Small airway epithelium none | 4.5 |
| Small airway epithelium TNFalpha + IL- | 29.1 |
| 1beta | |
| Coronery artery SMC rest | 9.9 |
| Coronery artery SMC TNFalpha + IL-1beta | 13.3 |
| Astrocytes rest | 2.6 |
| Astrocytes TNFalpha + IL-1beta | 4.2 |
| KU-812 (Basophil) rest | 4.9 |
| KU-812 (Basophil) PMA/ionomycin | 11.9 |
| CCD1106 (Keratinocytes) none | 28.3 |
| CCD1106 (Keratinocytes) TNFalpha + IL- | 18.6 |
| 1beta | |
| Liver cirrhosis | 4.6 |
| NCI-H292 none | 46.3 |
| NCI-H292 IL-4 | 46.0 |
| NCI-H292 IL-9 | 69.3 |
| NCI-H292 IL-13 | 59.0 |
| NCI-H292 IFN gamma | 33.9 |
| HPAEC none | 12.9 |
| HPAEC TNF alpha + IL-1 beta | 70.2 |
| Lung fibroblast none | 14.2 |
| Lung fibroblast TNF alpha + IL-1 beta | 20.0 |
| Lung fibroblast IL-4 | 12.4 |
| Lung fibroblast IL-9 | 4.8 |
| Lung fibroblast IL-13 | 2.7 |
| Lung fibroblast IFN gamma | 27.7 |
| Dermal fibroblast CCD1070 rest | 33.9 |
| Dermal fibroblast CCD1070 TNF alpha | 62.4 |
| Dermal fibroblast CCD1070 IL-1 beta | 18.3 |
| Dermal fibroblast IFN gamma | 19.3 |
| Dermal fibroblast IL-4 | 37.4 |
| Dermal Fibroblasts rest | 15.8 |
| Neutrophils TNFa + LPS | 37.6 |
| Neutrophils rest | 41.2 |
| Colon | 1.5 |
| Lung | 0.9 |
| Thymus | 2.4 |
| Kidney | 17.2 |

TABLE ABE
Panel 5 Islet

| | Rel. Exp. (%) |
|---|---|
| | Ag5630, Run |
| Tissue Name | 279370866 |
| 97457_Patient-02go_adipose | 15.5 |
| 97476_Patient-07sk_skeletal muscle | 0.0 |
| 97477_Patient-07ut_uterus | 5.0 |
| 97478_Patient-07pl_placenta | 9.3 |
| 99167_Bayer Patient 1 | 100.0 |
| 97482_Patient-08ut_uterus | 11.0 |
| 97483_Patient-08pl_placenta | 7.9 |
| 97486_Patient-09sk_skeletal muscle | 9.9 |
| 97487_Patient-09ut_uterus | 4.1 |
| 97488_Patient-09pl_placenta | 10.3 |
| 97492_Patient-10ut_uterus | 10.2 |
| 97493_Patient-10pl_placenta | 20.9 |
| 97495_Patient-11go_adipose | 5.8 |
| 97496_Patient-11sk_skeletal muscle | 4.4 |
| 97497_Patient-11ut_uterus | 13.5 |
| 97498_Patient-11pl_placenta | 3.4 |
| 97500_Patient-12go_adipose | 37.1 |
| 97501_Patient-12sk_skeletal muscle | 20.2 |
| 97502_Patient-12ut_uterus | 22.8 |
| 97503_Patient-12pl_placenta | 13.1 |
| 94721_Donor 2 U - A_Mesenchymal Stem | 87.7 |
| Cells | |
| 94722_Donor 2 U - B_Mesenchymal Stem | 75.8 |
| Cells | |
| 94723_Donor 2 U - C_Mesenchymal Stem | 77.9 |
| Cells | |
| 94709_Donor 2 AM - A_adipose | 26.6 |
| 94710_Donor 2 AM - B_adipose | 21.0 |
| 94711_Donor 2 AM - C_adipose | 16.7 |
| 94712_Donor 2 AD - A_adipose | 55.9 |
| 94713_Donor 2 AD - B_adipose | 74.7 |
| 94714_Donor 2 AD - C_adipose | 54.7 |
| 94742_Donor 3 U - A_Mesenchymal Stem | 5.7 |
| Cells | |
| 94743_Donor 3 U - B_Mesenchymal Stem | 8.0 |
| Cells | |
| 94730_Donor 3 AM - A_adipose | 8.3 |
| 94731_Donor 3 AM - B_adipose | 14.3 |
| 94732_Donor 3 AM - C_adipose | 11.3 |
| 94733_Donor 3 AD - A_adipose | 30.1 |
| 94734_Donor 3 AD - B_adipose | 22.5 |
| 94735_Donor 3 AD - C_adipose | 7.5 |
| 77138_Liver_HepG2untreated | 2.5 |
| 73556_Heart_Cardiac stromal cells | 2.7 |
| (primary) | |
| 81735_Small Intestine | 12.6 |
| 72409_Kidney_Proximal Convoluted Tubule | 28.1 |
| 82685_Small intestine_Duodenum | 24.0 |
| 90650_Adrenal_Adrenocortical adenoma | 7.3 |
| 72410_Kidney_HRCE | 33.0 |
| 72411_Kidney_HRE | 10.4 |
| 73139_Uterus_Uterine smooth muscle | 11.8 |
| cells | |

CNS_neurodegeneration_v1.0 Summary: Ag5630 This panel confirms the expression of this gene at low levels in the brains of an independent group of individuals. However, no differential expression of this gene was detected between Alzheimer's diseased postmortem brains and those of non-demented controls in this experiment. Please see Panel 1.5 for a discussion of this gene in treatment of central nervous system disorders.

General_screen_panel_v1.5 Summary: Ag5630 Higest expression of this gene is detected in a gastric cancer NCI-N87 cell line (CT=27.6). Moderate levels of expression of this gene is also seen in cluster of cancer cell lines derived from pancreatic, gastric, colon, lung, liver, renal, breast, ovarian, prostate, squamous cell carcinoma, melanoma and brain cancers. Thus, expression of this gene could be used as a marker to detect the presence of these cancers. Furthermore, therapeutic modulation of the expression or function of this gene may be effective in the treatment of pancreatic, gastric, colon, lung, liver, renal, breast, ovarian, prostate, squamous cell carcinoma, melanoma and brain cancers.

Among tissues with metabolic or endocrine function, this gene is expressed at moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

In addition, this gene is expressed at moderate levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

Panel 4.1D Summary: Ag5630 Higest expression of this gene is detected in LPS treated monocytes (CT=29.7). Interestingly, this gene is expressed at much higher levels in LPS activated when compared to resting monocytes (CT=40). This observation suggests that expression of this gene can be used to distinguish actvated from resting monocytes. In addition, upon activation monocytes contribute to the innate and specific immunity by migrating to the site of tissue injury and releasing inflammatory cytokines. This release contributes to the inflammation process. Therefore, modulation of the expression of the protein encoded by this gene may prevent the recruitment of monocytes and the initiation of the inflammatory process.

In addition, this gene is also expressed at moderate to low levels in activated polarized T cells, naive and memory T cells, resting and activated LAK cells, resting IL-2 treated NK cells, two way MLR, activated PBMC cells and B lymphocytes, dendritic cells, macrophage, different endothelial cells, bronchial and small airway epithelium, astrocytes, basophils, keratinocytes, mucoepidermoid cells, lung and dermal fibroblasts, neutrophils and kidney. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Panel 5 Islet Summary: Ag5630 Higest expression of this gene is detected in beta islet cells (CT=26.7). In addition, this gene shows widespread expression in this panel, with moderate to low expressions in adipose, placenta, uterus, skeletal muscle, kidney, and small intestine samples. Therefore, therapeutic modulation of this gene may be useful in the treatment of metabolic/endocrine disorders including, obesity, Type I and II diabetes.

AC. CG149350-01 and CG149350-02: Vacuolar ATP Synthase Subunit F.

Expression of gene CG149350-01 and CG149350-02 was assessed using the primer-probe set Ag7581, described in Table ACA. Results of the RTQ-PCR runs are shown in Table ACB. Please note that CG149350-02 represents a full-length physical clone of the CG149350-01 gene, validating the prediction of the gene sequence.

TABLE ACA

Probe Name Ag7581

| | | | Start | SEQ ID |
|---|---|---|---|---|
| Primers | Sequences | Length | Position | No |
| Forward | 5′-aagaactgccaccccaatt-3′ | 19 | 88 | 351 |
| Probe | TET-5′-cattgatggtcgtatccttctccacca-3′-TAMRA | 27 | 113 | 352 |
| Reverse | 5′-aaattgccggaaagtgtctt-3′ | 20 | 146 | 353 |

TABLE ACB
CNS_neurodegeneration_v1.0

| | Rel. Exp. (%) |
|---|---|
| | Ag7581, Run |
| Tissue Name | 308752174 |
| AD 1 Hippo | 19.9 |
| AD 2 Hippo | 21.3 |
| AD 3 Hippo | 14.9 |
| AD 4 Hippo | 6.4 |
| AD 5 hippo | 65.5 |
| AD 6 Hippo | 44.4 |
| Control 2 Hippo | 21.9 |
| Control 4 Hippo | 30.6 |
| Control (Path) 3 Hippo | 10.7 |
| AD 1 Temporal Ctx | 23.0 |
| AD 2 Temporal Ctx | 27.5 |
| AD 3 Temporal Ctx | 19.8 |
| AD 4 Temporal Ctx | 21.3 |
| AD 5 Inf Temporal Ctx | 46.3 |
| AD 5 Sup Temporal Ctx | 55.9 |
| AD 6 Inf Temporal Ctx | 52.9 |
| AD 6 Sup Temporal Ctx | 47.3 |
| Control 1 Temporal Ctx | 23.5 |
| Control 2 Temporal Ctx | 28.9 |
| Control 3 Temporal Ctx | 22.2 |
| Control 4 Temporal Ctx | 9.1 |
| Control (Path) 1 Temporal Ctx | 45.7 |
| Control (Path) 2 Temporal Ctx | 62.0 |
| Control (Path) 3 Temporal Ctx | 7.3 |
| Control (Path) 4 Temporal Ctx | 62.9 |
| AD 1 Occipital Ctx | 19.1 |
| AD 2 Occipital Ctx (Missing) | 0.0 |
| AD 3 Occipital Ctx | 22.4 |
| AD 4 Occipital Ctx | 32.3 |
| AD 5 Occipital Ctx | 4.4 |
| AD 6 Occipital Ctx | 20.2 |
| Control 1 Occipital Ctx | 3.0 |
| Control 2 Occipital Ctx | 35.6 |
| Control 3 Occipital Ctx | 53.2 |
| Control 4 Occipital Ctx | 6.8 |
| Control (Path) 1 Occipital Ctx | 70.7 |
| Control (Path) 2 Occipital Ctx | 17.9 |
| Control (Path) 3 Occipital Ctx | 4.2 |
| Control (Path) 4 Occipital Ctx | 32.5 |
| Control 1 Parietal Ctx | 8.7 |
| Control 2 Parietal Ctx | 56.3 |
| Control 3 Parietal Ctx | 32.5 |
| Control (Path) 1 Parietal Ctx | 100.0 |
| Control (Path) 2 Parietal Ctx | 38.4 |
| Control (Path) 3 Parietal Ctx | 17.6 |
| Control (Path) 4 Parietal Ctx | 64.2 |

CNS_neurodegeneration_v1.0 Summary: Ag7581 No differential expression of this gene was detected between Alzheimer's diseased postmortem brains and those of non-demented controls in this experiment. However, this panel confirms the expression of this gene at low levels in the brains of an independent group of individuals. Therefore, therapeutic modulation of this gene product may be useful in the treatment of central nervous system disorders such as Parkinson's disease, epilepsy, multiple sclerosis, schizophrenia and depression.

AD. CGI49536-01: Protein-Tyrosine Phosphatase, Non-Receptor Type 2.

Expression of gene CGI49536-01 was assessed using the primer-probe sets Ag5255 and Ag6844, described in Tables ADA and ADB. Results of the RTQ-PCR runs are shown in Tables ADC, ADD and ADE.

TABLE ADA

Probe Name Ag5255

| | | | Start | SEQ ID |
|---|---|---|---|---|
| Primers | | Length | Position | No |
| Forward | 5′-cttatggtttggcagcagaa-3′ | 20 | 355 | 354 |
| Probe | TET-5′-ccaaagcagttgtcatgctgaaccgc-3′-TAMRA | 26 | 377 | 355 |
| Reverse | 5′-tggtttcaccactcgattct-3′ | 20 | 414 | 356 |

TABLE ADB

Probe Name Ag6844

| | | | Start | SEQ ID |
|---|---|---|---|---|
| Primers | | Length | Position | No |
| Forward | 5′-agagaatcgagtggtgaaacc-3′ | 21 | 412 | 357 |
| Probe | TET-5′-actacctggccagattttggagtccc-3′-TAMRA | 26 | 457 | 358 |
| Reverse | 5′-aggagccagattctctcacttta-3′ | 23 | 516 | 359 |

TABLE ADC
CNS_neurodegeneration_v1.0

| | Rel. Exp. (%) |
|---|---|
| | Ag5255, Run |
| Tissue Name | 229929883 |
| AD 1 Hippo | 28.9 |
| AD 2 Hippo | 42.3 |
| AD 3 Hippo | 42.0 |
| AD 4 Hippo | 5.9 |
| AD 5 hippo | 92.7 |
| AD 6 Hippo | 29.7 |
| Control 2 Hippo | 52.5 |
| Control 4 Hippo | 22.4 |
| Control (Path) 3 Hippo | 17.9 |
| AD 1 Temporal Ctx | 39.5 |
| AD 2 Temporal Ctx | 56.3 |
| AD 3 Temporal Ctx | 23.3 |
| AD 4 Temporal Ctx | 10.9 |
| AD 5 Inf Temporal Ctx | 44.8 |
| AD 5 Sup Temporal Ctx | 53.2 |
| AD 6 Inf Temporal Ctx | 68.8 |
| AD 6 Sup Temporal Ctx | 100.0 |
| Control 1 Temporal Ctx | 13.4 |
| Control 2 Temporal Ctx | 34.4 |
| Control 3 Temporal Ctx | 84.1 |
| Control 4 Temporal Ctx | 18.4 |
| Control (Path) 1 Temporal Ctx | 41.2 |
| Control (Path) 2 Temporal Ctx | 58.6 |
| Control (Path) 3 Temporal Ctx | 21.0 |
| Control (Path) 4 Temporal Ctx | 38.7 |
| AD 1 Occipital Ctx | 45.4 |
| AD 2 Occipital Ctx (Missing) | 0.0 |
| AD 3 Occipital Ctx | 36.9 |
| AD 4 Occipital Ctx | 23.5 |
| AD 5 Occipital Ctx | 13.6 |
| AD 6 Occipital Ctx | 47.6 |
| Control 1 Occipital Ctx | 3.2 |
| Control 2 Occipital Ctx | 57.4 |
| Control 3 Occipital Ctx | 31.2 |
| Control 4 Occipital Ctx | 5.0 |
| Control (Path) 1 Occipital Ctx | 99.3 |
| Control (Path) 2 Occipital Ctx | 40.3 |
| Control (Path) 3 Occipital Ctx | 0.0 |
| Control (Path) 4 Occipital Ctx | 24.0 |
| Control 1 Parietal Ctx | 20.6 |
| Control 2 Parietal Ctx | 68.3 |
| Control 3 Parietal Ctx | 29.5 |
| Control (Path) 1 Parietal Ctx | 46.3 |
| Control (Path) 2 Parietal Ctx | 31.2 |
| Control (Path) 3 Parietal Ctx | 6.9 |
| Control (Path) 4 Parietal Ctx | 45.1 |

TABLE ADD
General_screening_panel_v1.5

| | Rel. Exp. (%) |
|---|---|
| | Ag5255, Run |
| Tissue Name | 230218532 |
| Adipose | 6.4 |
| Melanoma* Hs688(A).T | 9.5 |
| Melanoma* Hs688(B).T | 8.7 |
| Melanoma* M14 | 19.1 |
| Melanoma* LOXMVI | 25.5 |
| Melanoma* SK-MEL-5 | 18.8 |
| Squamous cell carcinoma SCC-4 | 24.0 |
| Testis Pool | 2.2 |
| Prostate ca.* (bone met) PC-3 | 33.9 |
| Prostate Pool | 4.1 |
| Placenta | 1.9 |
| Uterus Pool | 2.3 |
| Ovarian ca. OVCAR-3 | 19.6 |
| Ovarian ca. SK-OV-3 | 55.5 |
| Ovarian ca. OVCAR-4 | 8.5 |
| Ovarian ca. OVCAR-5 | 44.4 |
| Ovarian ca. IGROV-1 | 5.7 |
| Ovarian ca. OVCAR-8 | 7.8 |
| Ovary | 8.0 |
| Breast ca. MCF-7 | 38.2 |
| Breast ca. MDA-MB-231 | 13.4 |
| Breast ca. BT 549 | 51.8 |
| Breast ca. T47D | 5.4 |
| Breast ca. MDA-N | 7.0 |
| Breast Pool | 9.0 |
| Trachea | 1.0 |
| Lung | 5.7 |
| Fetal Lung | 17.1 |
| Lung ca. NCI-N417 | 1.0 |
| Lung ca. LX-1 | 12.6 |
| Lung ca. NCI-H146 | 16.6 |
| Lung ca. SHP-77 | 34.6 |
| Lung ca. A549 | 15.1 |
| Lung ca. NCI-H526 | 6.7 |
| Lung ca. NCI-H23 | 33.0 |
| Lung ca. NCI-H460 | 7.2 |
| Lung ca. HOP-62 | 26.2 |
| Lung ca. NCI-H522 | 35.1 |
| Liver | 0.9 |
| Fetal Liver | 7.2 |
| Liver ca. HepG2 | 9.7 |
| Kidney Pool | 7.3 |
| Fetal Kidney | 16.3 |
| Renal ca. 786-0 | 7.1 |
| Renal ca. A498 | 2.2 |
| Renal ca. ACHN | 9.2 |
| Renal ca. UO-31 | 6.5 |
| Renal ca. TK-10 | 18.8 |
| Bladder | 10.8 |
| Gastric ca. (liver met.) NCI-N87 | 50.3 |
| Gastric ca. KATO III | 60.3 |
| Colon ca. SW-948 | 5.8 |
| Colon ca. SW480 | 100.0 |
| Colon ca.* (SW480 met) SW620 | 23.3 |
| Colon ca. HT29 | 19.2 |
| Colon ca. HCT-116 | 46.7 |
| Colon ca. CaCo-2 | 49.3 |
| Colon cancer tissue | 5.7 |
| Colon ca. SW1116 | 3.5 |
| Colon ca. Colo-205 | 3.3 |
| Colon ca. SW-48 | 0.5 |
| Colon Pool | 5.9 |
| Small Intestine Pool | 5.7 |
| Stomach Pool | 3.2 |
| Bone Marrow Pool | 2.8 |
| Fetal Heart | 3.7 |
| Heart Pool | 0.7 |
| Lymph Node Pool | 4.1 |
| Fetal Skeletal Muscle | 5.8 |
| Skeletal Muscle Pool | 2.6 |
| Spleen Pool | 0.4 |
| Thymus Pool | 19.2 |
| CNS cancer (glio/astro) U87-MG | 26.4 |
| CNS cancer (glio/astro) U-118-MG | 33.2 |
| CNS cancer (neuro; met) SK-N-AS | 18.9 |
| CNS cancer (astro) SF-539 | 17.1 |
| CNS cancer (astro) SNB-75 | 12.2 |
| CNS cancer (glio) SNB-19 | 6.4 |
| CNS cancer (glio) SF-259 | 16.0 |
| Brain (Amygdala) Pool | 4.0 |
| Brain (cerebellum) | 33.2 |
| Brain (fetal) | 54.0 |
| Brain (Hippocampus) Pool | 4.7 |
| Cerebral Cortex Pool | 5.3 |
| Brain (Substantia nigra) Pool | 4.0 |
| Brain (Thalamus) Pool | 6.8 |
| Brain (whole) | 4.9 |
| Spinal Cord Pool | 7.0 |
| Adrenal Gland | 2.4 |
| Pituitary gland Pool | 2.1 |
| Salivary Gland | 1.5 |
| Thyroid (female) | 1.1 |
| Pancreatic ca. CAPAN2 | 66.4 |
| Pancreas Pool | 7.2 |

TABLE ADE
Panel 4.1D

| | Rel. Exp. (%) | Rel. Exp. (%) |
|---|---|---|
| | Ag5255, Run | Ag6844, Run |
| Tissue Name | 229851730 | 279029113 |
| Secondary Th1 act | 39.0 | 38.7 |
| Secondary Th2 act | 46.7 | 55.9 |
| Secondary Tr1 act | 15.7 | 18.9 |
| Secondary Th1 rest | 12.0 | 3.9 |
| Secondary Th2 rest | 0.0 | 5.3 |
| Secondary Tr1 rest | 0.0 | 9.2 |
| Primary Th1 act | 17.9 | 6.0 |
| Primary Th2 act | 15.0 | 33.7 |
| Primary Tr1 act | 18.2 | 22.7 |
| Primary Th1 rest | 0.0 | 1.9 |
| Primary Th2 rest | 5.0 | 1.5 |
| Primary Tr1 rest | 0.0 | 0.0 |
| CD45RA CD4 lymphocyte act | 32.1 | 13.9 |
| CD45RO CD4 lymphocyte act | 58.6 | 42.9 |
| CD8 lymphocyte act | 5.2 | 18.7 |
| Secondary CD8 lymphocyte | 10.9 | 5.5 |
| rest | | |
| Secondary CD8 lymphocyte | 0.0 | 4.4 |
| act | | |
| CD4 lymphocyte none | 6.7 | 3.4 |
| 2ry Th1/Th2/Tr1_anti-CD95 | 0.0 | 26.4 |
| CH11 | | |
| LAK cells rest | 19.1 | 14.7 |
| LAK cells IL-2 | 5.4 | 7.3 |
| LAK cells IL-2 + IL-12 | 7.9 | 1.0 |
| LAK cells IL-2 + IFN | 16.2 | 7.7 |
| gamma | | |
| LAK cells IL-2 + IL-18 | 5.1 | 8.0 |
| LAK cells PMA/ionomycin | 27.9 | 40.9 |
| NK Cells IL-2 rest | 27.9 | 40.3 |
| Two Way MLR 3 day | 18.2 | 27.0 |
| Two Way MLR 5 day | 23.3 | 2.1 |
| Two Way MLR 7 day | 4.5 | 1.7 |
| PBMC rest | 3.2 | 5.4 |
| PBMC PWM | 20.6 | 9.8 |
| PBMC PHA-L | 21.6 | 12.1 |
| Ramos (B cell) none | 40.3 | 4.8 |
| Ramos (B cell) ionomycin | 31.6 | 17.7 |
| B lymphocytes PWM | 26.6 | 6.0 |
| B lymphocytes CD40L and | 4.8 | 37.6 |
| IL-4 | | |
| EOL-1 dbcAMP | 62.9 | 74.2 |
| EOL-1 dbcAMP PMA/ionomycin | 45.4 | 15.1 |
| Dendritic cells none | 33.7 | 57.0 |
| Dendritic cells LPS | 21.0 | 15.2 |
| Dendritic cells anti-CD40 | 10.2 | 7.3 |
| Monocytes rest | 4.3 | 32.1 |
| Monocytes LPS | 69.7 | 100.0 |
| Macrophages rest | 17.0 | 3.8 |
| Macrophages LPS | 0.0 | 9.3 |
| HUVEC none | 5.9 | 28.7 |
| HUVEC starved | 28.1 | 8.5 |
| HUVEC IL-1beta | 39.8 | 9.6 |
| HUVEC IFN gamma | 12.5 | 15.9 |
| HUVEC TNF alpha + IFN | 21.0 | 8.4 |
| gamma | | |
| HUVEC TNF alpha + IL4 | 12.1 | 11.0 |
| HUVEC IL-11 | 13.6 | 4.4 |
| Lung Microvascular EC none | 25.2 | 18.4 |
| Lung Microvascular EC | 2.6 | 9.4 |
| TNFalpha + IL-1beta | | |
| Microvascular Dermal EC | 6.0 | 3.8 |
| none | | |
| Microsvasular Dermal EC | 0.0 | 3.7 |
| TNFalpha + IL-1beta | | |
| Bronchial epithelium | 9.3 | 10.2 |
| TNFalpha + IL1beta | | |
| Small airway epithelium | 0.0 | 10.0 |
| none | | |
| Small airway epithelium | 37.1 | 14.1 |
| TNFalpha + IL-1beta | | |
| Coronery artery SMC rest | 11.1 | 5.5 |
| Coronery artery SMC | 11.3 | 4.0 |
| TNFalpha + IL-1beta | | |
| Astrocytes rest | 0.0 | 1.1 |
| Astrocytes TNFalpha + IL- | 0.0 | 1.8 |
| 1beta | | |
| KU-812 (Basophil) rest | 38.4 | 17.2 |
| KU-812 (Basophil) PMA/ | 33.2 | 38.7 |
| ionomycin | | |
| CCD1106 (Keratinocytes) | 76.3 | 40.1 |
| none | | |
| CCD1106 (Keratinocytes) | 13.1 | 14.9 |
| TNFalpha + IL-1beta | | |
| Liver cirrhosis | 15.8 | 7.0 |
| NCI-H292 none | 35.1 | 20.2 |
| NCI-H292 IL-4 | 45.4 | 25.5 |
| NCI-H292 IL-9 | 60.7 | 31.2 |
| NCI-H292 IL-13 | 45.4 | 38.4 |
| NCI-H292 IFN gamma | 26.2 | 16.7 |
| HPAEC none | 5.6 | 6.3 |
| HPAEC TNFalpha + IL-1 beta | 21.5 | 12.1 |
| Lung fibroblast none | 22.5 | 12.2 |
| Lung fibroblast TNF | 6.3 | 8.2 |
| alpha + IL-1 beta | | |
| Lung fibroblast IL-4 | 16.0 | 13.5 |
| Lung fibroblast IL-9 | 15.9 | 11.9 |
| Lung fibroblast IL-13 | 0.0 | 5.8 |
| Lung fibroblast IFN gamma | 37.6 | 19.9 |
| Dermal fibroblast CCD1070 | 32.3 | 17.2 |
| rest | | |
| Dermal fibroblast CCD1070 | 100.0 | 54.7 |
| TNF alpha | | |
| Dermal fibroblast CCD1070 | 34.6 | 18.7 |
| IL-1 beta | | |
| Dermal fibroblast IFN | 17.1 | 12.7 |
| gamma | | |
| Dermal fibroblast IL-4 | 5.3 | 15.0 |
| Dermal Fibroblasts rest | 0.0 | 6.9 |
| Neutrophils TNFa + LPS | 0.0 | 2.7 |
| Neutrophils rest | 5.6 | 6.1 |
| Colon | 0.0 | 0.9 |
| Lung | 0.0 | 1.7 |
| Thymus | 15.2 | 18.2 |
| Kidney | 6.3 | 8.7 |

AI_comprehensive panel_v1.0 Summary: Ag5255 Expression of this gene is low/undetectable (CTs>35) across all of the samples on this panel.

CNS_neurodegeneration_v1.0 Summary: Ag5255 This panel confirms the expression of this gene at low levels in the brains of an independent group of individuals. However, no differential expression of this gene was detected between Alzheimer's diseased postmortem brains and those of non-demented controls in this experiment. Please see Panel 1.5 for a discussion of this gene in treatment of central nervous system disorders.

General_screen_panel_v1.5 Summary: Ag5255 Highest expression of this gene is detected in a colon cancer SW480 cell line (CT=31.6). Moderate to low levels of expression of this gene is also seen in cluster of cancer cell lines derived from pancreatic, gastric, colon, lung, liver, renal, breast, ovarian, prostate, squamous cell carcinoma, melanoma and brain cancers. Thus, expression of this gene could be used as a marker to detect the presence of these cancers. Furthermore, therapeutic modulation of the expression or function of this gene may be effective in the treatment of pancreatic, gastric, colon, lung, liver, renal, breast, ovarian, prostate, squamous cell carcinoma, melanoma and brain cancers.

In addition, this gene is expressed at moderate levels in cerebellum and fetal brain. Therefore, therapeutic modulation of this gene product may be useful in the treatment of central nervous system disorders such ataxia and autism.

Panel 4.1D Summary: Ag5255/Ag6844 Two experiments with different probe and primer sets are in good agreement. The highest expression of this gene is detected in TNF alpha activated dermal fibroblast and LPS activated monocytes (CTs=32.7-32.9). Moderate to low levels of expression of this gene is detected in activated polarized T cells, naive and memory T cells, PMA/ionomycin activated LAK cells, resting IL-2 treated NK cells, eosinophils, resting dendritic cells, activated basophils, resting keratinocyte, and activated mucoepidermoid NCI-H292 cells. Therefore, therapeutic modulation of this gene or its protein product may be useful in the treatment of autoimmune and inflammatory diseases such as asthma, allergies, inflammatory bowel disease, lupus erythematosus, psoriasis, rheumatoid arthritis, and osteoarthritis.

AE. CG149964-01: Brain Mitochondrial Carrier Protein-1.

Expression of gene CG149964-01 was assessed using the primer-probe set Ag7056, described in Table AEA.

TABLE AEA

Probe Name Ag7056

| | | | Start | SEQ ID |
|---|---|---|---|---|
| Primers | Sequences | Length | Position | No |
| Forward | 5′-tgtggttccaactgctcag-3′ | 19 | 617 | 360 |
| Probe | TET-5′-ctggtagctctactcctacaacgatggcag-3′-TAMRA | 30 | 640 | 361 |
| Reverse | 5′-agatccacatgtcccatcatt-3′ | 21 | 707 | 362 |

General_screen_panel_v1.6 Summary: Ag7056 Expression of this gene is low/undetectable in all samples on this panel (CTs>35).

AF. CG150799-01, CG150799-02 and CG150799-03: MASS1.

Expression of gene CG150799-01, CG150799-02 and CG150799-03 was assessed using the primer-probe sets Ag5242, Ag5243, Ag5244, Ag5245, Ag5247 and Ag5248, described in Tables AFA, AFB and AFC. Results of the RTQ-PCR runs are shown in Tables AFD, AFE, AFF, AFG, AFH and AFI. Please note that probe-primer sets Ag5243 is specific for CG150799-02 and probe-primer sets Ag5244 and Ag5245 are specific for CG150799-03.

TABLE AFA

Probe Name Ag5242

| | | | Start | SEQ ID |
|---|---|---|---|---|
| Primers | | Length | Position | No |
| Forward | 5′-acgaatcccatgtgacacttt-3′ | 21 | 3624 | 363 |
| Probe | TET-5′-cccttcattataaaaccttgggttcca-3′-TAMRA | 27 | 3645 | 364 |
| Reverse | 5′-tgactgttgtcttggcaatgt-3′ | 21 | 3681 | 365 |

TABLE AFB

Probe Name Ag5243

| | | | Start | SEQ ID |
|---|---|---|---|---|
| Primers | Sequences | Length | Position | No |
| Forward | 5′-gactccttccaaaggctatattgt-3′ | 24 | 8809 | 366 |
| Probe | TET-5′-cgattcaaggccctacaaatatctgcca-3′-TAMRA | 28 | 8849 | 367 |
| Reverse | 5′-ccatttctggttccgtgtcta-3′ | 21 | 8880 | 368 |

TABLE AFC

Probe Name g5244

| | | | Start | SEQ ID |
|---|---|---|---|---|
| Primers | Sequences | Length | Position | No |
| Forward | 5′-actgataattctattcctgaactgga-3′ | 26 | 4927 | 369 |
| Probe | TET-5′-agctctgctagatctatctacagatataacgctgtaaaatc-3′-TAMRA | 41 | 4992 | 370 |
| Reverse | 5′-aactcattatagatcatccaaaagga-3′ | 26 | 5036 | 371 |

TABLE AED

Probe Name g5245

| | | | Start | SEQ ID |
|---|---|---|---|---|
| Primers | Sequences | Length | Position | No |
| Forward | 5′-accttgttgatgactttgctaatg-3′ | 24 | 4320 | 372 |
| Probe | TET-5′-cagtggaactattacattccttccttggcaga-3′-TAMRA | 32 | 4345 | 373 |
| Reverse | 5′-ggaagcgacacttcaatcaaa-3′ | 21 | 4387 | 374 |

TABLE AFE

Probe Name Ag5247

| | | | Start | SEQ ID |
|---|---|---|---|---|
| Primers | Sequences | Length | Position | No |
| Forward | 5′-acttacgttggacttaccatgg-3′ | 22 | 8183 | 375 |
| Probe | TET-5′-caacttcatttcctcccagactaggtatgagg-3′-TAMRA | 32 | 8211 | 376 |
| Reverse | 5′-tcatttcatttgaagtgagcaa-3′ | 22 | 8263 | 377 |

TABLE AFF

Probe Name Ag5248

| | | | Start | SEQ ID |
|---|---|---|---|---|
| Primers | Sequences | Length | Position | No |
| Forward | 5′-accttgttgatgactttgctaatg-3′ | 24 | 4320 | 378 |
| Probe | TET-5′-cagtggaactattacattccttccttggcaga-3′-TAMRA | 32 | 4345 | 379 |
| Reverse | 5′-caagaacatatatattcagagaacctctgatc-3′ | 30 | 4377 | 380 |

TABLE AFG
AI_comprehensive_panel_v1.0

| | Rel. Exp. (%) |
|---|---|
| | Ag5242, Run |
| Tissue Name | 305464510 |
| 110967 COPD-F | 0.1 |
| 110980 COPD-F | 1.1 |
| 110968 COPD-M | 0.1 |
| 110977 COPD-M | 4.4 |
| 110989 Emphysema-F | 0.2 |
| 110992 Emphysema-F | 2.7 |
| 110993 Emphysema-F | 0.1 |
| 110994 Emphysema-F | 0.1 |
| 110995 Emphysema-F | 6.8 |
| 110996 Emphysema-F | 2.0 |
| 110997 Asthma-M | 0.1 |
| 111001 Asthma-F | 0.5 |
| 111002 Asthma-F | 0.9 |
| 111003 Atopic Asthma-F | 1.5 |
| 111004 Atopic Asthma-F | 6.1 |
| 111005 Atopic Asthma-F | 2.5 |
| 111006 Atopic Asthma-F | 0.9 |
| 111417 Allergy-M | 0.8 |
| 112347 Allergy-M | 0.0 |
| 112349 Normal Lung-F | 0.0 |
| 112357 Normal Lung-F | 1.0 |
| 112354 Normal Lung-M | 0.7 |
| 112374 Crohns-F | 0.5 |
| 112389 Match Control Crohns-F | 0.2 |
| 112375 Crohns-F | 0.1 |
| 112732 Match Control Crohns-F | 0.3 |
| 112725 Crohns-M | 0.1 |
| 112387 Match Control Crohns-M | 0.1 |
| 112378 Crohns-M | 0.0 |
| 112390 Match Control Crohns-M | 1.5 |
| 112726 Crohns-M | 1.2 |
| 112731 Match Control Crohns-M | 0.9 |
| 112380 Ulcer Col-F | 1.0 |
| 112734 Match Control Ulcer Col-F | 0.8 |
| 112384 Ulcer Col-F | 3.7 |
| 112737 Match Control Ulcer Col-F | 0.8 |
| 112386 Ulcer Col-F | 0.2 |
| 112738 Match Control Ulcer Col-F | 0.5 |
| 112381 Ulcer Col-M | 0.0 |
| 112735 Match Control Ulcer Col-M | 0.0 |
| 112382 Ulcer Col-M | 0.3 |
| 112394 Match Control Ulcer Col-M | 0.1 |
| 112383 Ulcer Col-M | 4.5 |
| 112736 Match Control Ulcer Col-M | 0.3 |
| 112423 Psoriasis-F | 0.2 |
| 112427 Match Control Psoriasis-F | 2.3 |
| 112418 Psoriasis-M | 0.1 |
| 112723 Match Control Psoriasis-M | 0.5 |
| 112419 Psoriasis-M | 0.0 |
| 112424 Match Control Psoriasis-M | 0.2 |
| 112420 Psoriasis-M | 1.8 |
| 112425 Match Control Psoriasis-M | 3.7 |
| 104689 (MF) OA Bone-Backus | 0.2 |
| 104690 (MF) Adj “Normal” Bone-Backus | 0.6 |
| 104691 (MF) OA Synovium-Backus | 0.1 |
| 104692 (BA) OA Cartilage-Backus | 0.0 |
| 104694 (BA) OA Bone-Backus | 0.2 |
| 104695 (BA) Adj “Normal” Bone-Backus | 0.4 |
| 104696 (BA) OA Synovium-Backus | 0.1 |
| 104700 (SS) OA Bone-Backus | 0.9 |
| 104701 (SS) Adj “Normal” Bone-Backus | 0.6 |
| 104702 (SS) OA Synovium-Backus | 0.2 |
| 117093 OA Cartilage Rep7 | 0.9 |
| 112672 OA Bone5 | 0.0 |
| 112673 OA Synovium5 | 0.1 |
| 112674 OA Synovial Fluid cells5 | 0.2 |
| 117100 OA Cartilage Rep14 | 0.0 |
| 112756 OA Bone9 | 100.0 |
| 112757 OA Synovium9 | 6.4 |
| 112758 OA Synovial Fluid Cells9 | 0.1 |
| 117125 RA Cartilage Rep2 | 0.0 |
| 113492 Bone2 RA | 31.6 |
| 113493 Synovium2 RA | 11.8 |
| 113494 Syn Fluid Cells RA | 22.2 |
| 113499 Cartilage4 RA | 22.7 |
| 113500 Bone4 RA | 28.1 |
| 113501 Synovium4 RA | 20.2 |
| 113502 Syn Fluid Cells4 RA | 16.4 |
| 113495 Cartilage3 RA | 22.7 |
| 113496 Bone3 RA | 24.5 |
| 113497 Synovium3 RA | 14.7 |
| 113498 Syn Fluid Cells3 RA | 33.0 |
| 117106 Normal Cartilage Rep20 | 0.0 |
| 113663 Bone3 Normal | 0.0 |
| 113664 Synovium3 Normal | 0.0 |
| 113665 Syn Fluid Cells3 Normal | 0.0 |
| 117107 Normal Cartilage Rep22 | 0.1 |
| 113667 Bone4 Normal | 0.4 |
| 113668 Synovium4 Normal | 0.1 |
| 113669 Syn Fluid Cells4 Normal | 0.8 |

TABLE AFH
CNS_neurodegeneration_v1.0

| | Rel. | Rel. | Rel. | Rel. | Rel. | Rel. | Rel. | Rel. |
|---|---|---|---|---|---|---|---|---|
| | Exp. | Exp. | Exp. | Exp. | Exp. | Exp. | Exp. | Exp. |
| | (%) | (%) | (%) | (%) | (%) | (%) | (%) | (%) |
| | Ag5242, | Ag5242, | Ag5243, | Ag5243, | Ag5243, | Ag5244, | Ag5244, | Ag5244, |
| Tissue | Run | Run | Run | Run | Run | Run | Run | Run |
| Name | 229661546 | 233609876 | 229661547 | 276863566 | 277731460 | 229661548 | 233610762 | 277731461 |
| AD 1 | 22.4 | 21.6 | 29.3 | 31.6 | 27.5 | 9.1 | 0.0 | 3.1 |
| Hippo | | | | | | | | |
| AD 2 | 47.3 | 42.0 | 54.7 | 53.2 | 44.8 | 0.0 | 2.9 | 4.0 |
| Hippo | | | | | | | | |
| AD 3 | 12.2 | 13.5 | 17.8 | 13.6 | 10.9 | 0.0 | 0.0 | 0.0 |
| Hippo | | | | | | | | |
| AD 4 | 14.8 | 14.4 | 16.6 | 17.7 | 20.6 | 0.0 | 0.0 | 0.0 |
| Hippo | | | | | | | | |
| AD 5 | 65.5 | 84.1 | 61.6 | 63.7 | 57.4 | 6.7 | 0.0 | 4.3 |
| Hippo | | | | | | | | |
| AD 6 | 56.3 | 59.5 | 82.4 | 84.7 | 90.1 | 74.2 | 57.8 | 51.8 |
| Control 2 | 29.5 | 25.7 | 29.3 | 31.6 | 31.9 | 0.0 | 0.0 | 5.5 |
| Hippo | | | | | | | | |
| Control 4 | 32.8 | 29.7 | 35.6 | 31.2 | 37.1 | 8.1 | 11.3 | 0.0 |
| Hippo | | | | | | | | |
| Control | 33.9 | 33.9 | 24.7 | 24.0 | 30.8 | 0.0 | 4.5 | 0.0 |
| (Path) 3 | | | | | | | | |
| Hippo | | | | | | | | |
| AD 1 | 32.3 | 33.9 | 32.3 | 34.6 | 35.4 | 2.0 | 5.4 | 2.9 |
| Temporal | | | | | | | | |
| Ctx | | | | | | | | |
| AD 2 | 35.8 | 42.3 | 39.5 | 51.1 | 46.3 | 3.3 | 5.4 | 0.0 |
| Temporal | | | | | | | | |
| Ctx | | | | | | | | |
| AD 3 | 28.3 | 21.2 | 20.4 | 23.5 | 20.7 | 0.0 | 0.0 | 0.0 |
| Temporal | | | | | | | | |
| Ctx | | | | | | | | |
| AD 4 | 47.3 | 44.8 | 36.6 | 39.0 | 45.4 | 10.3 | 0.0 | 8.3 |
| Temporal | | | | | | | | |
| Ctx | | | | | | | | |
| AD 5 | 73.7 | 100.0 | 100.0 | 100.0 | 100.0 | 0.0 | 11.4 | 17.6 |
| Inf | | | | | | | | |
| Temporal | | | | | | | | |
| Ctx | | | | | | | | |
| AD 5 | 93.3 | 77.4 | 87.7 | 82.4 | 88.3 | 7.3 | 10.7 | 8.9 |
| Sup | | | | | | | | |
| Temporal | | | | | | | | |
| Ctx | | | | | | | | |
| AD 6 | 59.0 | 58.2 | 62.0 | 0.0 | 58.2 | 55.9 | 94.0 | 49.0 |
| Inf | | | | | | | | |
| Temporal | | | | | | | | |
| Ctx | | | | | | | | |
| AD 6 | 85.3 | 99.3 | 74.2 | 74.7 | 90.1 | 100.0 | 100.0 | 100.0 |
| Sup | | | | | | | | |
| Temporal | | | | | | | | |
| Ctx | | | | | | | | |
| Control 1 | 47.6 | 46.3 | 27.4 | 28.5 | 29.1 | 1.7 | 0.0 | 0.0 |
| Temporal | | | | | | | | |
| Ctx | | | | | | | | |
| Control 2 | 37.6 | 37.4 | 30.6 | 27.5 | 32.8 | 2.7 | 11.0 | 4.5 |
| Temporal | | | | | | | | |
| Ctx | | | | | | | | |
| Control 3 | 27.5 | 24.1 | 27.4 | 32.8 | 37.6 | 7.1 | 5.4 | 2.6 |
| Temporal | | | | | | | | |
| Ctx | | | | | | | | |
| Control 3 | 38.2 | 39.0 | 34.6 | 30.6 | 31.9 | 8.7 | 2.6 | 4.9 |
| Temporal | | | | | | | | |
| Ctx | | | | | | | | |
| Control | 66.0 | 81.2 | 54.0 | 58.6 | 52.5 | 2.5 | 0.0 | 3.2 |
| (Path) 1 | | | | | | | | |
| Temporal | | | | | | | | |
| Ctx | | | | | | | | |
| Control | 43.5 | 50.0 | 40.1 | 41.8 | 41.5 | 2.0 | 10.9 | 0.0 |
| (Path) 2 | | | | | | | | |
| Temporal | | | | | | | | |
| Ctx | | | | | | | | |
| Control | 23.3 | 24.5 | 19.9 | 21.5 | 22.4 | 2.9 | 2.3 | 7.7 |
| (Path) 3 | | | | | | | | |
| Temporal | | | | | | | | |
| Ctx | | | | | | | | |
| Control | 52.5 | 48.0 | 33.7 | 39.8 | 39.0 | 0.0 | 4.7 | 4.3 |
| (Path) 4 | | | | | | | | |
| Temporal | | | | | | | | |
| Ctx | | | | | | | | |
| AD 1 | 18.0 | 18.8 | 22.8 | 25.7 | 24.3 | 0.0 | 3.0 | 0.0 |
| Occipital | | | | | | | | |
| Ctx | | | | | | | | |
| AD 2 | 0.0 | 0.0 | 0.0 | 0.0 | 0.0 | 0.0 | 0.0 | 0.0 |
| Occipital | | | | | | | | |
| Ctx | | | | | | | | |
| (Missing) | | | | | | | | |
| AD 3 | 15.5 | 14.0 | 17.8 | 17.8 | 18.0 | 0.0 | 0.0 | 0.0 |
| Occipital | | | | | | | | |
| Ctx | | | | | | | | |
| AD 4 | 17.3 | 23.7 | 25.3 | 27.5 | 24.3 | 3.3 | 3.1 | 3.3 |
| Occipital | | | | | | | | |
| Ctx | | | | | | | | |
| AD 5 | 22.4 | 26.1 | 21.3 | 15.2 | 22.5 | 2.0 | 3.1 | 5.3 |
| Occipital | | | | | | | | |
| Ctx | | | | | | | | |
| AD 6 | 28.9 | 21.6 | 19.1 | 20.4 | 18.9 | 11.7 | 15.6 | 2.8 |
| Occipital | | | | | | | | |
| Ctx | | | | | | | | |
| Control 1 | 9.3 | 10.2 | 6.8 | 6.1 | 7.4 | 0.0 | 0.0 | 0.0 |
| Occipital | | | | | | | | |
| Ctx | | | | | | | | |
| Control 2 | 34.9 | 33.0 | 24.7 | 28.7 | 31.6 | 0.0 | 5.1 | 7.4 |
| Occipital | | | | | | | | |
| Ctx | | | | | | | | |
| Control 3 | 27.2 | 24.1 | 27.5 | 25.2 | 24.5 | 2.4 | 9.2 | 4.2 |
| Occipital | | | | | | | | |
| Ctx | | | | | | | | |
| Control 4 | 19.6 | 20.3 | 18.0 | 26.8 | 21.2 | 0.0 | 1.6 | 0.0 |
| Occipital | | | | | | | | |
| Ctx | | | | | | | | |
| Control | 56.6 | 64.6 | 48.6 | 58.6 | 57.8 | 9.1 | 5.1 | 7.8 |
| (Path) 1 | | | | | | | | |
| Occipital | | | | | | | | |
| Ctx | | | | | | | | |
| Control | 5.7 | 6.1 | 9.0 | 7.1 | 8.5 | 2.0 | 0.0 | 0.0 |
| (Path) 2 | | | | | | | | |
| Occipital | | | | | | | | |
| Ctx | | | | | | | | |
| Control | 2.6 | 3.1 | 4.1 | 1.9 | 4.5 | 0.0 | 0.0 | 0.0 |
| (Path) 3 | | | | | | | | |
| Occipital | | | | | | | | |
| Ctx | | | | | | | | |
| Control | 9.9 | 11.2 | 5.4 | 9.2 | 8.2 | 0.0 | 0.0 | 0.0 |
| (Path) 4 | | | | | | | | |
| Occipital | | | | | | | | |
| Ctx | | | | | | | | |
| Control 1 | 28.9 | 32.5 | 19.6 | 21.8 | 22.4 | 0.0 | 0.0 | 0.0 |
| Parietal | | | | | | | | |
| Ctx | | | | | | | | |
| Control 2 | 100.0 | 90.8 | 79.0 | 83.5 | 76.3 | 7.9 | 23.8 | 9.7 |
| Parietal | | | | | | | | |
| Ctx | | | | | | | | |
| Control 3 | 14.8 | 11.9 | 17.3 | 15.3 | 17.0 | 0.0 | 9.8 | 0.0 |
| Parietal | | | | | | | | |
| Ctx | | | | | | | | |
| Control | 62.4 | 68.3 | 57.8 | 70.2 | 63.7 | 4.2 | 3.8 | 0.0 |
| (Path) 1 | | | | | | | | |
| Parietal | | | | | | | | |
| Ctx | | | | | | | | |
| Control | 17.1 | 19.8 | 22.1 | 21.0 | 25.9 | 1.9 | 10.4 | 0.0 |
| (Path) 2 | | | | | | | | |
| Parietal | | | | | | | | |
| Ctx | | | | | | | | |
| Control | 12.0 | 10.2 | 11.7 | 8.4 | 13.9 | 2.8 | 5.3 | 0.0 |
| (Path) 3 | | | | | | | | |
| Parietal | | | | | | | | |
| Ctx | | | | | | | | |
| Control | 30.1 | 25.5 | 26.1 | 25.7 | 29.1 | 1.5 | 0.0 | 0.0 |
| (Path) 4 | | | | | | | | |
| Parietal | | | | | | | | |
| Ctx | | | | | | | | |

| | Rel. | Rel. | Rel. | Rel. | Rel. | Rel. | Rel. |
|---|---|---|---|---|---|---|---|
| | Exp. | Exp. | Exp. | Exp. | Exp. | Exp. | Exp. |
| | (%) | (%) | (%) | (%) | (%) | (%) | (%) |
| | Ag5245, | Ag5245, | Ag5247, | Ag5247, | Ag5248, | Ag5248, | Ag5248, |
| Tissue | Run | Run | Run | Run | Run | Run | Run |
| Name | 229661549 | 230510320 | 229661550 | 276863570 | 229661551 | 276863572 | 277731466 |
| AD 1 | 16.0 | 0.0 | 9.0 | 6.7 | 14.9 | 13.7 | 17.9 |
| Hippo | | | | | | | |
| AD 2 | 16.2 | 4.6 | 41.8 | 21.8 | 44.4 | 32.8 | 32.5 |
| Hippo | | | | | | | |
| AD 3 | 0.0 | 0.0 | 5.8 | 0.0 | 9.8 | 4.8 | 6.8 |
| Hippo | | | | | | | |
| AD 4 | 23.2 | 7.6 | 17.3 | 8.6 | 12.8 | 6.4 | 7.0 |
| Hippo | | | | | | | |
| AD 5 | 11.6 | 5.3 | 84.7 | 31.0 | 85.3 | 61.1 | 62.0 |
| Hippo | | | | | | | |
| AD 6 | 58.6 | 30.8 | 100.0 | 92.0 | 69.3 | 48.3 | 55.5 |
| Hippo | | | | | | | |
| Control 2 | 15.1 | 29.1 | 42.0 | 29.9 | 27.7 | 25.0 | 26.1 |
| Hippo | | | | | | | |
| Control 4 | 0.0 | 0.0 | 27.0 | 23.7 | 25.2 | 20.9 | 16.5 |
| Hippo | | | | | | | |
| Control | 8.2 | 0.0 | 13.0 | 12.2 | 13.1 | 19.8 | 22.1 |
| (Path) 3 | | | | | | | |
| Hippo | | | | | | | |
| AD 1 | 9.3 | 19.5 | 29.9 | 21.9 | 26.2 | 17.9 | 26.1 |
| Temporal | | | | | | | |
| Ctx | | | | | | | |
| AD 2 | 14.2 | 0.0 | 28.7 | 32.5 | 38.2 | 37.6 | 100.0 |
| Temporal | | | | | | | |
| Ctx | | | | | | | |
| AD 3 | 0.0 | 0.0 | 4.4 | 4.5 | 12.2 | 9.5 | 11.3 |
| Temporal | | | | | | | |
| Ctx | | | | | | | |
| AD 4 | 39.0 | 19.2 | 43.5 | 25.3 | 33.0 | 25.9 | 29.5 |
| Temporal | | | | | | | |
| Ctx | | | | | | | |
| AD 5 | 24.5 | 0.0 | 43.5 | 74.7 | 76.8 | 100.0 | 79.6 |
| Inf | | | | | | | |
| Temporal | | | | | | | |
| Ctx | | | | | | | |
| AD 5 | 29.1 | 3.3 | 45.7 | 59.0 | 82.4 | 70.7 | 64.2 |
| Sup | | | | | | | |
| Temporal | | | | | | | |
| Ctx | | | | | | | |
| AD 6 | 55.5 | 100.0 | 87.1 | 87.7 | 71.7 | 46.3 | 65.1 |
| Inf | | | | | | | |
| Temporal | | | | | | | |
| Ctx | | | | | | | |
| AD 6 | 99.3 | 73.7 | 95.9 | 97.3 | 97.3 | 60.3 | 94.0 |
| Sup | | | | | | | |
| Temporal | | | | | | | |
| Ctx | | | | | | | |
| Control 1 | 58.2 | 27.7 | 25.3 | 19.1 | 44.4 | 25.2 | 32.3 |
| Temporal | | | | | | | |
| Ctx | | | | | | | |
| Control 2 | 31.4 | 48.3 | 8.9 | 15.4 | 50.0 | 34.4 | 29.1 |
| Temporal | | | | | | | |
| Ctx | | | | | | | |
| Control 3 | 5.1 | 6.3 | 16.6 | 5.8 | 35.6 | 21.3 | 27.7 |
| Temporal | | | | | | | |
| Ctx | | | | | | | |
| Control 3 | 8.4 | 0.0 | 31.4 | 13.7 | 22.4 | 26.1 | 31.4 |
| Temporal | | | | | | | |
| Ctx | | | | | | | |
| Control | 78.5 | 37.9 | 72.7 | 72.7 | 75.8 | 63.3 | 69.7 |
| (Path) 1 | | | | | | | |
| Temporal | | | | | | | |
| Ctx | | | | | | | |
| Control | 80.1 | 20.9 | 42.6 | 31.9 | 42.9 | 33.9 | 42.0 |
| (Path) 2 | | | | | | | |
| Temporal | | | | | | | |
| Ctx | | | | | | | |
| Control | 0.0 | 4.1 | 5.8 | 4.3 | 21.0 | 14.5 | 16.2 |
| (Path) 3 | | | | | | | |
| Temporal | | | | | | | |
| Ctx | | | | | | | |
| Control | 49.3 | 43.2 | 73.7 | 49.3 | 40.6 | 32.5 | 47.6 |
| (Path) 4 | | | | | | | |
| Temporal | | | | | | | |
| Ctx | | | | | | | |
| AD 1 | 0.0 | 0.0 | 10.2 | 13.8 | 19.9 | 12.8 | 14.3 |
| Occipital | | | | | | | |
| Ctx | | | | | | | |
| AD 2 | 0.0 | 0.0 | 0.0 | 0.0 | 0.0 | 0.0 | 0.0 |
| Occipital | | | | | | | |
| Ctx | | | | | | | |
| (Missing) | | | | | | | |
| AD 3 | 3.2 | 0.0 | 10.2 | 0.0 | 10.3 | 5.2 | 5.5 |
| Occipital | | | | | | | |
| Ctx | | | | | | | |
| AD 4 | 28.7 | 6.7 | 22.2 | 23.0 | 8.6 | 17.0 | 21.5 |
| Occipital | | | | | | | |
| Ctx | | | | | | | |
| AD 5 | 25.7 | 5.1 | 16.7 | 8.7 | 3.3 | 20.9 | 18.4 |
| Occipital | | | | | | | |
| Ctx | | | | | | | |
| AD 6 | 0.0 | 18.2 | 12.1 | 0.0 | 29.9 | 18.0 | 24.7 |
| Occipital | | | | | | | |
| Ctx | | | | | | | |
| Control 1 | 4.3 | 7.8 | 5.6 | 0.0 | 4.8 | 3.7 | 3.3 |
| Occipital | | | | | | | |
| Ctx | | | | | | | |
| Control 2 | 31.2 | 17.9 | 7.9 | 4.6 | 39.5 | 20.2 | 28.3 |
| Occipital | | | | | | | |
| Ctx | | | | | | | |
| Control 3 | 7.0 | 0.0 | 13.8 | 0.0 | 14.5 | 17.6 | 16.8 |
| Occipital | | | | | | | |
| Ctx | | | | | | | |
| Control 4 | 0.0 | 0.0 | 12.5 | 5.6 | 15.4 | 8.8 | 12.9 |
| Occipital | | | | | | | |
| Ctx | | | | | | | |
| Control | 66.4 | 30.6 | 69.7 | 57.0 | 76.3 | 42.6 | 55.5 |
| (Path) 1 | | | | | | | |
| Occipital | | | | | | | |
| Ctx | | | | | | | |
| Control | 5.6 | 0.0 | 1.6 | 0.0 | 16.3 | 3.8 | 4.1 |
| (Path) 2 | | | | | | | |
| Occipital | | | | | | | |
| Ctx | | | | | | | |
| Control | 0.0 | 0.0 | 0.0 | 0.0 | 1.5 | 1.8 | 1.7 |
| (Path) 3 | | | | | | | |
| Occipital | | | | | | | |
| Ctx | | | | | | | |
| Control | 11.7 | 15.7 | 5.1 | 7.2 | 2.1 | 0.3 | 5.0 |
| (Path) 4 | | | | | | | |
| Occipital | | | | | | | |
| Ctx | | | | | | | |
| Control 1 | 0.0 | 3.6 | 9.8 | 16.4 | 23.8 | 16.3 | 17.1 |
| Parietal | | | | | | | |
| Ctx | | | | | | | |
| Control 2 | 26.8 | 12.2 | 39.0 | 37.9 | 100.0 | 44.1 | 63.3 |
| Parietal | | | | | | | |
| Ctx | | | | | | | |
| Control 3 | 0.0 | 0.0 | 1.7 | 7.2 | 12.9 | 8.5 | 11.3 |
| Parietal | | | | | | | |
| Ctx | | | | | | | |
| Control | 100.0 | 55.9 | 41.5 | 100.0 | 99.3 | 53.2 | 71.2 |
| (Path) 1 | | | | | | | |
| Parietal | | | | | | | |
| Ctx | | | | | | | |
| Control | 30.8 | 0.0 | 17.9 | 18.0 | 6.3 | 10.2 | 15.8 |
| (Path) 2 | | | | | | | |
| Parietal | | | | | | | |
| Ctx | | | | | | | |
| Control | 6.3 | 0.0 | 3.9 | 0.0 | 3.2 | 4.9 | 4.2 |
| (Path) 3 | | | | | | | |
| Parietal | | | | | | | |
| Ctx | | | | | | | |
| Control | 59.0 | 40.9 | 23.7 | 30.1 | 25.9 | 26.4 | 17.8 |
| (Path) 4 | | | | | | | |
| Parietal | | | | | | | |
| Ctx | | | | | | | |

TABLE AFI
General_screening_panel_v1.5

| | Rel. | Rel. | Rel. | Rel. | Rel. |
|---|---|---|---|---|---|
| | Exp. | Exp. | Exp. | Exp. | Exp. |
| | (%) | (%) | (%) | (%) | (%) |
| | Ag5242, | Ag5243, | Ag5245, | Ag5247, | Ag5248, |
| Tissue | Run | Run | Run | Run | Run |
| Name | 229665046 | 229665047 | 229665049 | 229665052 | 229665053 |
| Adipose | 0.1 | 0.0 | 0.0 | 0.0 | 0.0 |
| Melanoma* | 0.8 | 0.5 | 0.0 | 0.0 | 1.2 |
| Hs688(A).T | | | | | |
| Melanoma* | 0.1 | 0.0 | 0.0 | 0.0 | 0.0 |
| Hs688(B).T | | | | | |
| Melanoma* M14 | 0.2 | 0.3 | 0.0 | 0.0 | 0.1 |
| Melanoma* | 0.9 | 0.2 | 0.0 | 0.0 | 0.1 |
| LOXIMVI | | | | | |
| Melanoma* | 0.6 | 1.6 | 0.0 | 1.2 | 0.0 |
| SK-MEL-5 | | | | | |
| Squamous cell | 0.0 | 0.0 | 0.0 | 0.0 | 0.0 |
| carcinoma SCC-4 | | | | | |
| Testis | 2.9 | 3.4 | 0.0 | 3.3 | 3.8 |
| Pool | | | | | |
| Prostate ca.* | 89.5 | 86.5 | 5.8 | 18.2 | |
| (bone met) PC-3 | | | | | |
| Prostate | 10.7 | 8.5 | 0.7 | 2.4 | 7.0 |
| Pool | | | | | |
| Placenta | 0.0 | 0.1 | 0.0 | 1.0 | 0.1 |
| Uterus | 0.0 | 0.1 | 0.0 | 0.0 | 0.1 |
| Pool | | | | | |
| Ovarian ca. | 10.5 | 18.7 | 12.1 | 1.7 | |
| OVCAR-3 | | | | | |
| Ovarian ca. | 0.2 | 0.1 | 0.0 | 0.2 | 0.0 |
| SK-OV-3 | | | | | |
| Ovarian ca. | 0.1 | 0.0 | 0.0 | 0.0 | 0.1 |
| OVCAR-4 | | | | | |
| Ovarian ca. | 7.3 | 7.1 | 0.0 | 3.7 | 12.1 |
| OVCAR-5 | | | | | |
| Ovarian ca. | 1.4 | 3.5 | 0.0 | 0.0 | 0.5 |
| IGROV-1 | | | | | |
| Ovarian ca. | 8.5 | 13.0 | 0.9 | 0.5 | 10.7 |
| OVCAR-8 | | | | | |
| Ovary | 0.1 | 0.4 | 0.0 | 0.0 | 1.0 |
| Breast ca. | 11.1 | 10.2 | 0.0 | 3.6 | 16.4 |
| MCF-7 | | | | | |
| Breast ca. | 3.7 | 4.8 | 3.2 | 0.6 | 5.9 |
| MDA-MB-231 | | | | | |
| Breast ca. | 0.0 | 0.0 | 0.0 | 0.0 | 0.0 |
| BT 549 | | | | | |
| Breast ca. | 10.2 | 4.4 | 0.0 | 3.1 | 9.9 |
| T47D | | | | | |
| Breast ca. | 0.1 | 0.2 | 0.0 | 0.0 | 0.5 |
| MDA-N | | | | | |
| Breast | 0.8 | 1.9 | 0.0 | 0.9 | 1.5 |
| Pool | | | | | |
| Trachea | 7.4 | 6.4 | 0.9 | 20.3 | 9.5 |
| Lung | 0.3 | 0.0 | 0.0 | 0.0 | 0.1 |
| Fetal | 25.7 | 20.9 | 1.7 | 6.7 | 22.2 |
| Lung | | | | | |
| Lung ca. | 3.4 | 3.6 | 0.0 | 0.7 | 11.6 |
| NCI-N417 | | | | | |
| Lung ca. | 0.1 | 0.0 | 0.0 | 0.0 | 0.0 |
| LX-1 | | | | | |
| Lung ca. | 26.1 | 28.9 | 27.9 | 7.7 | 24.7 |
| NCI-H146 | | | | | |
| Lung ca. | 100.0 | 100.0 | 100.0 | 42.9 | 98.6 |
| SHP-77 | | | | | |
| Lung ca. | 0.9 | 1.3 | 0.0 | 0.0 | 1.1 |
| A549 | | | | | |
| Lung ca. | 1.8 | 1.1 | 0.0 | 0.0 | 1.9 |
| NCI-H526 | | | | | |
| Lung ca. | 0.0 | 0.0 | 0.0 | 0.0 | 0.2 |
| NCI-H23 | | | | | |
| Lung ca. | 5.4 | 3.3 | 9.3 | 48.3 | 23.5 |
| NCI-H460 | | | | | |
| Lung ca. | 7.0 | 8.8 | 0.0 | 0.0 | 8.4 |
| HOP-62 | | | | | |
| Lung ca. | 0.0 | 0.0 | 0.0 | 0.0 | 0.0 |
| NCI-H522 | | | | | |
| Liver | 0.0 | 0.0 | 0.0 | 0.0 | 0.0 |
| Fetal | 0.0 | 0.0 | 0.0 | 0.6 | 0.2 |
| Liver | | | | | |
| Liver ca. | 0.0 | 0.1 | 0.0 | 0.0 | 0.0 |
| HepG2 | | | | | |
| Kidney | 1.0 | 1.0 | 0.0 | 0.4 | 1.6 |
| Pool | | | | | |
| Fetal | 8.5 | 6.9 | 1.0 | 6.5 | 9.2 |
| Kidney | | | | | |
| Renal ca. | 0.0 | 0.0 | 0.0 | 0.0 | 0.0 |
| 786-0 | | | | | |
| Renal ca. | 0.1 | 0.0 | 0.0 | 0.0 | 0.0 |
| A498 | | | | | |
| Renal ca. | 0.4 | 0.1 | 0.0 | 0.0 | 0.5 |
| ACHN | | | | | |
| Renal ca. | 0.0 | 0.1 | 0.0 | 0.0 | 0.0 |
| UO-31 | | | | | |
| Renal ca. | 0.0 | 0.1 | 0.0 | 0.0 | 0.0 |
| TK-10 | | | | | |
| Bladder | 2.6 | 1.8 | 0.0 | 2.5 | 3.7 |
| Gastric ca. | 0.0 | 0.0 | 0.0 | 0.0 | 0.0 |
| (liver met.) | | | | | |
| NCI-N87 | | | | | |
| Gastric ca. | 0.0 | 0.0 | 0.0 | 0.0 | 0.1 |
| KATO III | | | | | |
| Colon ca. | 5.2 | 4.6 | 0.4 | 0.6 | 3.7 |
| SW-948 | | | | | |
| Colon ca. | 4.6 | 3.7 | 0.0 | 1.1 | 5.9 |
| SW480 | | | | | |
| Colon ca.* | 0.1 | 0.0 | 0.0 | 0.0 | 0.0 |
| (SW480 met) | | | | | |
| SW620 | | | | | |
| Colon ca. | 0.0 | 0.0 | 0.0 | 0.0 | 0.0 |
| HT29 | | | | | |
| Colon ca. | 12.2 | 11.9 | 0.4 | 4.4 | 14.2 |
| HTC-116 | | | | | |
| Colon ca. | 13.8 | 14.0 | 8.9 | 6.6 | 16.4 |
| CaCo-2 | | | | | |
| Colon | 0.0 | 0.0 | 0.0 | 0.0 | 0.0 |
| cancer | | | | | |
| tissue | | | | | |
| Colon ca. | 0.1 | 0.0 | 0.0 | 0.0 | 0.0 |
| SW1116 | | | | | |
| Colon ca. | 0.0 | 0.0 | 0.0 | 0.0 | 1.2 |
| Colo-205 | | | | | |
| Colon ca. | 0.0 | 0.0 | 0.0 | 0.0 | 0.0 |
| SW-48 | | | | | |
| Colon | 0.1 | 0.0 | 0.0 | 0.6 | 0.1 |
| Pool | | | | | |
| Small | 3.7 | 1.6 | 1.6 | 1.0 | 4.1 |
| Intestine | | | | | |
| Pool | | | | | |
| Stomach | 1.6 | 0.7 | 0.0 | 0.4 | 0.9 |
| Pool | | | | | |
| Bone | 0.1 | 0.0 | 0.0 | 0.0 | 0.1 |
| Marrow | | | | | |
| Pool | | | | | |
| Fetal | 0.0 | 0.0 | 0.0 | 0.3 | 0.0 |
| Heart | | | | | |
| Heart Pool | 0.1 | 0.0 | 0.0 | 0.7 | 0.1 |
| Lymph | 0.5 | 0.0 | 0.0 | 0.6 | 0.1 |
| Node Pool | | | | | |
| Fetal | 0.2 | 0.0 | 0.0 | 1.1 | 0.0 |
| Skeletal | | | | | |
| Muscle | | | | | |
| Skeletal | 0.0 | 0.1 | 0.0 | 0.8 | 0.1 |
| Muscle | | | | | |
| Pool | | | | | |
| Spleen | 1.5 | 0.1 | 0.5 | 2.3 | 0.6 |
| Pool | | | | | |
| Thymus | 3.2 | 1.7 | 1.9 | 0.7 | 2.9 |
| Pool | | | | | |
| CNS cancer | 4.4 | 2.6 | 0.3 | 1.2 | 3.2 |
| (glio/astro) | | | | | |
| U87-MG | | | | | |
| CNS cancer | 0.1 | 0.0 | 0.0 | 0.0 | 0.0 |
| (glio/astro) | | | | | |
| U-118-MG | | | | | |
| CNS cancer | 0.0 | 0.0 | 0.0 | 0.0 | 0.0 |
| (neuro; met) | | | | | |
| SK-N-AS | | | | | |
| CNS cancer | 0.2 | 0.0 | 0.0 | 0.0 | 0.1 |
| (astro) | | | | | |
| SF-539 | | | | | |
| CNS cancer | 0.1 | 0.1 | 0.0 | 0.0 | 0.2 |
| (astro) | | | | | |
| SNB-75 | | | | | |
| CNS cancer (glio) | 2.0 | 4.1 | 0.0 | 0.6 | 3.4 |
| SNB-19 | | | | | |
| CNS cancer (glio) | 2.4 | 3.3 | 0.4 | 0.3 | 4.1 |
| SF-295 | | | | | |
| Brain | 13.4 | 29.1 | 1.8 | 4.2 | 14.6 |
| (Amygdala) | | | | | |
| Pool | | | | | |
| Brain | 14.2 | 13.4 | 0.8 | 6.1 | 15.6 |
| (cerebellum) | | | | | |
| Brain | 89.5 | 100.0 | 15.1 | 100.0 | 93.3 |
| (fetal) | | | | | |
| Brain | 35.4 | 47.3 | 6.6 | 13.7 | 31.9 |
| (Hippocampus) | | | | | |
| Pool | | | | | |
| Cerebral | 40.1 | 53.2 | 8.9 | 35.1 | 39.0 |
| Cortex | | | | | |
| Pool | | | | | |
| Brain | 14.2 | 33.7 | 4.7 | 2.2 | 16.7 |
| (Substantia | | | | | |
| nigra) | | | | | |
| Pool | | | | | |
| Brain | 37.9 | 43.2 | 0.8 | 25.5 | 45.1 |
| (Thalamus) | | | | | |
| Pool | | | | | |
| Brain | 13.9 | 25.7 | 2.1 | 13.4 | 18.6 |
| (whole) | | | | | |
| Spinal | 2.2 | 2.6 | 1.7 | 1.4 | 2.4 |
| Cord Pool | | | | | |
| Adrenal | 0.7 | 0.7 | 0.8 | 1.9 | 0.3 |
| Gland | | | | | |
| Pituitary | 18.6 | 16.3 | 5.0 | 3.2 | 36.6 |
| gland Pool | | | | | |
| Salivary | 0.1 | 0.5 | 0.0 | 0.0 | 0.1 |
| Gland | | | | | |
| Thyroid | 11.6 | 12.2 | 0.2 | 0.7 | 9.4 |
| (female) | | | | | |
| Pancreatic ca. | 0.1 | 0.0 | 0.0 | 0.0 | 0.1 |
| CAPAN2 | | | | | |
| Pancreas | 3.6 | 2.3 | 0.0 | 1.7 | 2.0 |
| Pool | | | | | |

TABLE AFJ
General_screening_panel_v1.6

| | Rel. | Rel. | Rel. | Rel. | Rel. | Rel. | Rel. | Rel. |
|---|---|---|---|---|---|---|---|---|
| | Exp. (%) | Exp. (%) | Exp. (%) | Exp. (%) | Exp. (%) | Exp. (%) | Exp. (%) | Exp. (%) |
| | Ag5243, | Ag5243, | Ag5245, | Ag5245, | Ag5247, | Ag5247, | Ag5248, | Ag5248, |
| | Run | Run | Run | Run | Run | Run | Run | Run |
| Tissue Name | 277218719 | 277729929 | 277219697 | 277730879 | 277219699 | 277729933 | 277219701 | 277730881 |
| Adipose | 0.1 | 0.2 | 0.0 | 0.0 | 0.0 | 0.0 | 0.1 | 0.1 |
| Melanoma* | 0.0 | 0.0 | 0.0 | 0.0 | 0.0 | 0.0 | 0.0 | 0.0 |
| Hs688(A).T | | | | | | | | |
| Melanoma* | 0.0 | 0.0 | 0.0 | 0.0 | 0.0 | 0.0 | 0.2 | 0.0 |
| Hs688(B).T | | | | | | | | |
| Melanoma* | 0.2 | 0.0 | 0.7 | 0.0 | 0.0 | 0.0 | 0.0 | 0.3 |
| M14 | | | | | | | | |
| Melanoma* | 0.2 | 0.1 | 0.0 | 0.0 | 0.0 | 0.0 | 0.1 | 0.2 |
| LOXIMVI | | | | | | | | |
| Melanoma* | 2.5 | 1.3 | 0.0 | 0.0 | 0.0 | 0.9 | 0.1 | 0.4 |
| SK-MEL-5 | | | | | | | | |
| Squamous cell | 0.0 | 0.0 | 0.0 | 0.0 | 0.0 | 0.0 | 0.0 | 0.1 |
| carcinoma | | | | | | | | |
| SCC-4 | | | | | | | | |
| Testis Pool | 2.2 | 3.4 | 3.1 | 2.3 | 7.1 | 3.5 | 2.7 | 2.8 |
| Prostate ca.* | 95.3 | 76.8 | 11.5 | 1.3 | 23.7 | 20.3 | 76.8 | 63.3 |
| (bone met) | | | | | | | | |
| PC-3 | | | | | | | | |
| Prostate Pool | 6.8 | 7.5 | 0.0 | 0.0 | 6.3 | 8.7 | 6.1 | 7.0 |
| Placenta | 0.0 | 0.0 | 0.0 | 0.0 | 0.0 | 0.0 | 0.1 | 0.1 |
| Uterus Pool | 0.0 | 0.0 | 0.0 | 0.0 | 0.0 | 0.0 | 0.0 | 0.1 |
| Ovarian ca. | 13.2 | 11.7 | 9.5 | 4.0 | 3.3 | 5.2 | 11.6 | 14.5 |
| OVCAR-3 | | | | | | | | |
| Ovarian ca. | 0.2 | 0.3 | 0.0 | 0.0 | 0.0 | 0.0 | 0.1 | 0.3 |
| SK-OV-3 | | | | | | | | |
| Ovarian ca. | 0.0 | 0.0 | 0.0 | 0.0 | 0.0 | 0.0 | 0.0 | 0.0 |
| OVCAR-4 | | | | | | | | |
| Ovarian ca. | 6.6 | 7.4 | 2.3 | 0.0 | 4.7 | 0.8 | 4.7 | 5.1 |
| OVCAR-5 | | | | | | | | |
| Ovarian ca. | 2.0 | 2.8 | 0.7 | 0.0 | 0.0 | 0.0 | 1.1 | 3.3 |
| IGROV-1 | | | | | | | | |
| Ovarian ca. | 14.2 | 8.1 | 3.6 | 0.0 | 7.5 | 8.1 | 8.2 | 13.4 |
| OVCAR-8 | | | | | | | | |
| Ovary | 0.1 | 0.6 | 0.0 | 0.0 | 0.0 | 0.0 | 0.7 | 0.2 |
| Breast ca. | 7.4 | 8.0 | 0.0 | 0.0 | 3.5 | 9.4 | 8.0 | 9.2 |
| MCF-7 | | | | | | | | |
| Breast ca. | 6.5 | 3.0 | 2.4 | 2.5 | 0.4 | 0.7 | 4.1 | 6.4 |
| MDA-MB-231 | | | | | | | | |
| Breast ca. BT 549 | 0.0 | 0.0 | 0.0 | 0.0 | 0.0 | 1.0 | 0.0 | 0.0 |
| Breast ca. | 6.7 | 3.8 | 0.8 | 0.0 | 5.5 | 1.5 | 4.7 | 8.0 |
| T47D | | | | | | | | |
| Breast ca. | 0.0 | 0.2 | 0.5 | 0.0 | 0.0 | 0.5 | 0.1 | 0.3 |
| MDA-N | | | | | | | | |
| Breast Pool | 0.2 | 0.1 | 0.9 | 0.0 | 0.0 | 0.0 | 0.5 | 0.3 |
| Trachea | 18.6 | 15.6 | 3.9 | 0.0 | 14.6 | 18.0 | 5.5 | 7.6 |
| Lung | 0.2 | 0.0 | 0.0 | 0.0 | 0.0 | 1.2 | 0.0 | 0.1 |
| Fetal Lung | 21.3 | 21.0 | 0.0 | 0.7 | 10.3 | 5.1 | 19.3 | 23.7 |
| Lung ca. | 6.3 | 3.2 | 0.0 | 0.0 | 1.7 | 4.2 | 2.4 | 2.0 |
| NCI-N417 | | | | | | | | |
| Lung ca. LX-1 | 0.0 | 0.0 | 0.0 | 0.0 | 0.0 | 0.0 | 0.0 | 0.0 |
| Lung ca. | 23.3 | 20.4 | 17.0 | 100.0 | 7.1 | 9.8 | 16.8 | 16.4 |
| NCI-H146 | | | | | | | | |
| Lung ca. | 95.9 | 77.9 | 100.0 | 35.6 | 24.7 | 31.9 | 100.0 | 76.3 |
| SHP-77 | | | | | | | | |
| Lung ca. A549 | 1.0 | 0.4 | 0.0 | 0.0 | 0.0 | 0.0 | 0.3 | 1.1 |
| Lung ca. | 1.4 | 1.9 | 0.0 | 0.0 | 0.0 | 0.0 | 0.7 | 0.5 |
| NCI-H526 | | | | | | | | |
| Lung ca. | 0.0 | 0.0 | 0.0 | 0.0 | 0.0 | 0.0 | 0.0 | 0.1 |
| NCI-H23 | | | | | | | | |
| Lung ca. | 2.8 | 2.1 | 0.0 | 0.0 | 0.9 | 0.9 | 3.1 | 3.4 |
| NCI-H460 | | | | | | | | |
| Lung ca. | 12.4 | 6.5 | 0.0 | 0.0 | 0.6 | 0.0 | 9.4 | 11.6 |
| HOP-62 | | | | | | | | |
| Lung ca. | 0.0 | 0.0 | 0.0 | 0.0 | 0.0 | 0.0 | 0.0 | 0.0 |
| NCI-H522 | | | | | | | | |
| Liver | 0.0 | 0.0 | 0.0 | 0.0 | 0.0 | 0.0 | 0.0 | 0.0 |
| Fetal | 0.0 | 0.0 | 0.0 | 0.0 | 0.0 | 0.9 | 0.2 | 0.0 |
| Liver | | | | | | | | |
| Liver ca. | 0.2 | 0.0 | 0.0 | 0.0 | 0.0 | 0.0 | 0.0 | 0.1 |
| HepG2 | | | | | | | | |
| Kidney | 0.5 | 0.9 | 0.0 | 0.0 | 1.0 | 0 0 | 0.6 | 1.8 |
| Pool | | | | | | | | |
| Fetal | 5.8 | 6.8 | 0.0 | 0.0 | 11.4 | 6.6 | 4.3 | 7.9 |
| Kidney | | | | | | | | |
| Renal ca. | 0.0 | 0.0 | 0.0 | 0.0 | 0.0 | 0.0 | 0.0 | 0.0 |
| 786-0 | | | | | | | | |
| Renal ca. | 0.0 | 0.0 | 0.0 | 0.0 | 0.0 | 0.0 | 0.0 | 0.0 |
| A498 | | | | | | | | |
| Renal ca. | 0.0 | 0.2 | 0.0 | 0.0 | 0.0 | 0.0 | 0.2 | 0.1 |
| ACHN | | | | | | | | |
| Renal ca. | 0.2 | 0.2 | 0.0 | 0.0 | 0.0 | 0.0 | 0.0 | 0.1 |
| UO-31 | | | | | | | | |
| Renal ca. | 0.0 | 0.0 | 0.0 | 0.0 | 0.0 | 0.0 | 0.0 | 0.0 |
| TK-10 | | | | | | | | |
| Bladder | 1.2 | 1.5 | 0.0 | 0.0 | 3.8 | 1.4 | 3.3 | 3.2 |
| Gastric ca. | 0.0 | 0.0 | 0.0 | 0.0 | 0.0 | 0.0 | 0.0 | 0.0 |
| (liver met.) | | | | | | | | |
| NCI-N87 | | | | | | | | |
| Gastric ca. | 0.0 | 0.0 | 0.0 | 0.0 | 0.0 | 0.0 | 0.0 | 0.0 |
| KATO III | | | | | | | | |
| Colon ca. | 4.0 | 4.4 | 0.7 | 0.0 | 2.8 | 0.6 | 3.6 | 3.8 |
| SW-948 | | | | | | | | |
| Colon ca. | 3.6 | 4.0 | 0.5 | 0.0 | 0.0 | 2.3 | 2.7 | 4.2 |
| SW480 | | | | | | | | |
| Colon ca.* | 0.2 | 0.0 | 0.0 | 0.0 | 0.0 | 0.0 | 0.0 | 0.0 |
| (SW480 met) | | | | | | | | |
| SW620 | | | | | | | | |
| Colon ca. | 0.0 | 0.0 | 0.0 | 0.0 | 0.0 | 0.0 | 0.0 | 0.0 |
| HT29 | | | | | | | | |
| Colon ca. | 13.8 | 12.7 | 1.0 | 0.0 | 6.8 | 3.1 | 5.6 | 14.7 |
| HCT-116 | | | | | | | | |
| Colon ca. | 18.8 | 14.9 | 10.8 | 4.7 | 10.2 | 10.1 | 2.4 | 11.6 |
| CaCo-2 | | | | | | | | |
| Colon cancer | 0.0 | 0.0 | 0.0 | 0.0 | 0.0 | 0.0 | 0.0 | 0.1 |
| tissue | | | | | | | | |
| Colon ca. | 0.0 | 0.0 | 0.0 | 0.0 | 0.0 | 0.0 | 0.0 | 0.0 |
| SW1116 | | | | | | | | |
| Colon ca. | 0.0 | 0.0 | 0.0 | 0.0 | 0.0 | 0.0 | 0.0 | 0.0 |
| Colo-205 | | | | | | | | |
| Colon ca. | 0.0 | 0.0 | 0.0 | 0.0 | 0.0 | 0.0 | 0.0 | 0.0 |
| SW-48 | | | | | | | | |
| Colon Pool | 0.1 | 0.0 | 0.0 | 0.0 | 0.9 | 0.0 | 0.4 | 0.1 |
| Small | 0.7 | 1.4 | 1.6 | 1.6 | 0.7 | 3.0 | 8.9 | 1.7 |
| Intestine | | | | | | | | |
| Pool | | | | | | | | |
| Stomach | 0.6 | 1.0 | 0.0 | 0.0 | 0.0 | 0.0 | 0.7 | 0.6 |
| Pool | | | | | | | | |
| Bone | 0.0 | 0.1 | 0.0 | 0.0 | 0.0 | 0.6 | 0.0 | 0.1 |
| Marrow | | | | | | | | |
| Pool | | | | | | | | |
| Fetal | 0.0 | 0.0 | 0.0 | 0.0 | 0.0 | 0.0 | 0.0 | 0.1 |
| Heart | | | | | | | | |
| Heart Pool | 0.0 | 0.0 | 0.0 | 0.0 | 0.0 | 0.0 | 0.0 | 0.0 |
| Lymph | 0.0 | 0.7 | 0.0 | 0.0 | 0.8 | 0.0 | 0.5 | 0.4 |
| Node Pool | | | | | | | | |
| Fetal | 0.4 | 0.1 | 0.0 | 0.0 | 0.0 | 0.0 | 0.1 | 0.2 |
| Skeletal | | | | | | | | |
| Muscle | | | | | | | | |
| Skeletal | 0.0 | 0.0 | 0.0 | 0.0 | 0.0 | 0.0 | 0.0 | 0.0 |
| Muscle | | | | | | | | |
| Pool | | | | | | | | |
| Spleen | 0.0 | 0.1 | 0.6 | 0.0 | 1.4 | 0.0 | 0.6 | 0.5 |
| Pool | | | | | | | | |
| Thymus | 2.0 | 2.1 | 1.0 | 0.7 | 1.4 | 2.6 | 1.9 | 3.2 |
| Pool | | | | | | | | |
| CNS cancer | 2.6 | 2.5 | 0.8 | 0.0 | 0.7 | 0.6 | 3.7 | 4.3 |
| (glio/astro) | | | | | | | | |
| U87-MG | | | | | | | | |
| CNS cancer | 0.3 | 0.1 | 0.0 | 0.0 | 0.0 | 0.0 | 0.0 | 0.0 |
| (glio/astro) | | | | | | | | |
| U-118-MG | | | | | | | | |
| CNS cancer | 0.0 | 0.0 | 0.0 | 0.0 | 0.0 | 0.0 | 0.0 | 0.0 |
| (neuro; met) | | | | | | | | |
| SK-N-AS | | | | | | | | |
| CNS cancer | 0.0 | 0.0 | 0.0 | 0.0 | 0.0 | 0.0 | 0.1 | 0.1 |
| (astro) SF-539 | | | | | | | | |
| CNS cancer | 0.2 | 0.3 | 0.0 | 0.0 | 0.0 | 0.0 | 0.5 | 0.3 |
| (astro) | | | | | | | | |
| SNB-75 | | | | | | | | |
| CNS cancer | 3.1 | 2.4 | 0.0 | 0.0 | 0.0 | 1.1 | 1.9 | 3.4 |
| (glio) SNB-19 | | | | | | | | |
| CNS cancer | 2.8 | 2.2 | 0.5 | 0.6 | 0.9 | 2.6 | 3.1 | 2.8 |
| (glio) SF-295 | | | | | | | | |
| Brain | 23.2 | 18.7 | 1.0 | 2.6 | 7.1 | 2.2 | 12.2 | 14.0 |
| (Amygdala) | | | | | | | | |
| Pool | | | | | | | | |
| Brain | 13.8 | 11.7 | 3.1 | 1.0 | 10.2 | 11.3 | 13.3 | 14.1 |
| (cerebellum) | | | | | | | | |
| Brain (fetal) | 100.0 | 100.0 | 20.6 | 14.8 | 100.0 | 100.0 | 73.2 | 100.0 |
| Brain | 51.1 | 40.3 | 6.9 | 5.3 | 25.9 | 14.3 | 26.8 | 35.8 |
| (Hippocampus) | | | | | | | | |
| Pool | | | | | | | | |
| Cerebral | 52.5 | 52.5 | 8.2 | 0.0 | 27.0 | 20.9 | 31.9 | 31.0 |
| Cortex | | | | | | | | |
| Pool | | | | | | | | |
| Brain | 29.5 | 29.1 | 1.1 | 1.7 | 5.5 | 2.9 | 9.7 | 12.2 |
| (Substantia | | | | | | | | |
| nigra) Pool | | | | | | | | |
| Brain | 48.3 | 51.1 | 2.2 | 2.5 | 21.9 | 25.2 | 17.4 | 31.0 |
| (Thalamus) | | | | | | | | |
| Pool | | | | | | | | |
| Brain (whole) | 28.7 | 30.6 | 6.0 | 4.2 | 15.2 | 13.3 | 9.2 | 14.7 |
| Spinal | 1.9 | 1.3 | 1.3 | 0.0 | 1.0 | 0.0 | 1.6 | 2.2 |
| Cord Pool | | | | | | | | |
| Adrenal | 0.4 | 0.8 | 1.5 | 0.0 | 0.0 | 0.8 | 0.3 | 0.4 |
| Gland | | | | | | | | |
| Pituitary | 17.9 | 13.7 | 2.6 | 7.4 | 0.0 | 11.1 | 13.4 | 15.8 |
| gland Pool | | | | | | | | |
| Salivary | 0.2 | 0.3 | 0.0 | 0.0 | 0.0 | 0.0 | 0.3 | 0.6 |
| Gland | | | | | | | | |
| Thyroid | 12.9 | 10.0 | 1.4 | 0.0 | 1.5 | 0.8 | 8.5 | 13.9 |
| (female) | | | | | | | | |
| Pancreatic ca. | 0.0 | 0.0 | 0.0 | 0.0 | 0.0 | 0.0 | 0.1 | 0.0 |
| CAPAN2 | | | | | | | | |
| Pancreas | 2.6 | 3.2 | 0.0 | 0.0 | 0.6 | 3.6 | 4.5 | 3.7 |
| Pool | | | | | | | | |

TABLE AFK
Panel 4.1D

| | Rel. | Rel. | Rel. | Rel. |
|---|---|---|---|---|
| | Exp. (%) | Exp. (%) | Exp. (%) | Exp. (%) |
| | Ag5242, | Ag5245, | Ag5247, | Ag5248, |
| | Run | Run | Run | Run |
| Tissue Name | 229819771 | 229819577 | 229819792 | 229819793 |
| Secondary Th1 act | 0.0 | 0.0 | 0.0 | 0.0 |
| Secondary Th2 act | 0.6 | 4.1 | 0.7 | 0.5 |
| Secondary Tr1 act | 2.3 | 1.2 | 0.6 | 2.3 |
| Secondary Th1 rest | 0.0 | 0.0 | 0.0 | 0.1 |
| Secondary Th2 rest | 13.7 | 0.6 | 5.1 | 12.2 |
| Secondary Tr1 rest | 15.5 | 1.9 | 8.7 | 14.0 |
| Primary Th1 act | 100.0 | 71.7 | 100.0 | 85.3 |
| Primary Th2 act | 27.9 | 12.6 | 20.4 | 28.3 |
| Primary Tr1 act | 36.6 | 9.4 | 24.3 | 28.9 |
| Primary Th1 rest | 15.9 | 2.9 | 5.1 | 14.6 |
| Primary Th2 rest | 34.2 | 3.4 | 23.3 | 29.1 |
| Primary Tr1 rest | 12.0 | 5.0 | 12.7 | 12.9 |
| CD45RA CD4 | 0.6 | 0.0 | 0.0 | 0.0 |
| lymphocyte act | | | | |
| CD45RO CD4 | 0.0 | 0.0 | 0.0 | 0.2 |
| lymphocyte act | | | | |
| CD8 lymphocyte act | 5.6 | 2.9 | 0.7 | 7.3 |
| Secondary CD8 | 0.0 | 0.0 | 0.0 | 0.0 |
| lymphocyte rest | | | | |
| Secondary CD8 | 2.1 | 0.0 | 0.0 | 1.9 |
| lymphocyte act | | | | |
| CD4 lymphocyte none | 8.1 | 1.2 | 5.8 | 7.4 |
| 2ry | 0.0 | 0.0 | 0.0 | 0.0 |
| Th1/Th2/Tr1_anti- | | | | |
| CD95 CH11 | | | | |
| LAK cells rest | 0.1 | 0.0 | 0.6 | 0.1 |
| LAK cells IL-2 | 0.3 | 0.0 | 0.0 | 0.3 |
| LAK cells | 25.2 | 3.1 | 4.8 | 24.0 |
| IL-2 + IL-12 | | | | |
| LAK cells | 0.2 | 0.0 | 0.0 | 1.1 |
| IL-2 + IFN gamma | | | | |
| LAK cells IL-2 + IL-18 | 0.5 | 0.0 | 0.7 | 0.7 |
| LAK cells | 0.2 | 0.0 | 0.6 | 0.0 |
| PMA/ionomycin | | | | |
| NK Cells IL-2 rest | 0.5 | 1.9 | 0.0 | 0.5 |
| Two Way MLR 3 day | 4.5 | 5.1 | 0.7 | 2.3 |
| Two Way MLR 5 day | 6.7 | 14.9 | 9.5 | 15.0 |
| Two Way MLR 7 day | 0.2 | 0.0 | 0.0 | 0.1 |
| PBMC rest | 8.7 | 0.0 | 2.3 | 6.0 |
| PBMC PWM | 0.2 | 0.0 | 0.0 | 0.4 |
| PBMC PHA-L | 0.2 | 0.0 | 0.0 | 0.1 |
| Ramos (B cell) none | 3.6 | 2.2 | 1.1 | 1.9 |
| Ramos (B cell) ionomycin | 1.8 | 3.6 | 1.5 | 2.2 |
| B lymphocytes PWM | 1.3 | 0.0 | 2.0 | 1.1 |
| B lymphocytes | 0.8 | 0.7 | 1.2 | 1.5 |
| CD40L and IL-4 | | | | |
| EOL-1 dbcAMP | 3.7 | 6.7 | 3.3 | 2.0 |
| EOL-1 dbcAMP | 3.0 | 0.0 | 2.3 | 2.0 |
| PMA/ionomycin | | | | |
| Dendritic cells none | 10.7 | 1.9 | 3.8 | 13.6 |
| Dendritic cells LPS | 4.7 | 6.2 | 11.7 | 8.2 |
| Dendritic cells anti-CD40 | 0.1 | 0.0 | 0.0 | 0.0 |
| Monocytes rest | 11.6 | 0.6 | 2.8 | 16.4 |
| Monocytes LPS | 4.6 | 5.6 | 1.4 | 5.4 |
| Macrophages rest | 0.2 | 0.0 | 0.0 | 0.1 |
| Macrophages LPS | 11.5 | 0.0 | 0.9 | 9.2 |
| HUVEC none | 0.3 | 0.0 | 0.0 | 0.5 |
| HUVEC starved | 15.9 | 8.4 | 2.4 | 15.5 |
| HUVEC IL-1beta | 0.2 | 0.0 | 0.0 | 0.1 |
| HUVEC IFN gamma | 0.0 | 0.0 | 0.0 | 0.0 |
| HUVEC TNF alpha +IFN gamma | 6.0 | 0.0 | 2.4 | 7.7 |
| HUVEC TNF alpha + IL4 | 1.0 | 0.0 | 0.6 | 4.2 |
| HUVEC IL-11 | 9.6 | 1.6 | 6.4 | 9.2 |
| Lung Microvascular EC none | 3.6 | 0.9 | 1.0 | 2.4 |
| Lung Microvascular | 0.0 | 0.0 | 0.0 | 0.0 |
| EC TNF alpha + IL-1beta | | | | |
| Microvascular | 0.1 | 0.0 | 0.0 | 0.3 |
| Dermal EC none | | | | |
| Microvascular | 0.0 | 0.0 | 0.0 | 0.0 |
| Dermal EC | | | | |
| TNF alpha + IL-1beta | | | | |
| Bronchial epithelium | 0.1 | 0.0 | 0.0 | 0.0 |
| TNF alpha + IL1beta | | | | |
| Small airway | 0.2 | 0.0 | 0.0 | 0.2 |
| epithelium none | | | | |
| Small airway | 3.1 | 0.0 | 0.7 | 3.7 |
| epithelium TNF alpha + IL-1beta | | | | |
| Coronery artery | 4.1 | 0.0 | 0.6 | 3.6 |
| SMC rest | | | | |
| Coronery artery | 3.1 | 0.0 | 0.0 | 2.6 |
| SMC TNF alpha + IL-1beta | | | | |
| Astrocytes rest | 3.8 | 0.9 | 0.6 | 4.0 |
| Astrocytes TNF alpha + IL-1beta | 0.0 | 0.0 | 0.0 | 0.0 |
| KU-812 (Basophil) rest | 0.0 | 0.0 | 0.0 | 0.0 |
| KU-812 (Basophil) | 12.6 | 1.0 | 4.5 | 15.4 |
| PMA/ionomycin | | | | |
| CCD1106 | 15.7 | 15.5 | 4.3 | 15.8 |
| (Keratinocytes) none | | | | |
| CCD1106 | 0.0 | 0.0 | 0.0 | 0.0 |
| (Keratinocytes) | | | | |
| TNF alpha + IL-1beta | | | | |
| Liver cirrhosis | 0.1 | 0.0 | 0.0 | 0.0 |
| NCI-H292 none | 0.0 | 0.0 | 0.0 | 0.0 |
| NCI-H292 IL-4 | 0.0 | 0.0 | 0.0 | 0.0 |
| NCI-H292 IL-9 | 0.0 | 0.0 | 0.0 | 0.0 |
| NCI-H292 IL-13 | 0.2 | 0.0 | 0.0 | 0.0 |
| NCI-H292 IFN gamma | 0.0 | 0.0 | 0.0 | 0.0 |
| HPAEC none | 0.0 | 0.0 | 0.0 | 0.0 |
| HPAEC TNF alpha + IL-1beta | 0.1 | 0.0 | 0.0 | 0.0 |
| Lung fibroblast none | 0.0 | 0.0 | 0.0 | 0.0 |
| Lung fibroblast TNF | 19.9 | 25.7 | 4.4 | 22.8 |
| alpha + IL-1beta | | | | |
| Lung fibroblast IL-4 | 72.2 | 100.0 | 32.8 | 49.7 |
| Lung fibroblast IL-9 | 1.2 | 0.0 | 0.4 | 0.6 |
| Lung fibroblast IL-13 | 1.8 | 0.0 | 1.5 | 1.2 |
| Lung fibroblast IFN gamma | 0.0 | 0.0 | 0.0 | 0.0 |
| Dermal fibroblast | 0.1 | 0.0 | 0.0 | 0.0 |
| CCD1070 rest | | | | |
| Dermal fibroblast | 2.9 | 0.0 | 1.3 | 5.3 |
| CCD1070 TNF alpha | | | | |
| Dermal fibroblast | 6.3 | 0.0 | 1.7 | 7.7 |
| CCD1070 IL-1beta | | | | |
| Dermal fibroblast | 0.0 | 0.0 | 0.0 | 0.0 |
| IFN gamma | | | | |
| Dermal fibroblast IL4 | 0.0 | 0.0 | 0.0 | 0.0 |
| Dermal Fibroblasts rest | 0.0 | 0.0 | 0.0 | 0.0 |
| Neutrophils | 0.1 | 0.0 | 0.0 | 0.0 |
| TNFa + LPS | | | | |
| Neutrophils rest | 87.7 | 11.7 | 28.3 | 100.0 |
| Colon | 0.0 | 0.0 | 0.0 | 0.0 |
| Lung | 0.2 | 0.0 | 0.0 | 0.3 |
| Thymus | 0.1 | 0.0 | 0.0 | 0.6 |
| Kidney | 0.1 | 0.0 | 1.4 | 0.6 |

TABLE AFL
general oncology screening panel_v_2.4

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag5242, | Ag5247, | Ag5248, | | Ag5242, | Ag5247, | Ag5248, |
| | Run | Run | Run | | Run | Run | Run |
| Tissue Name | 260269083 | 260269132 | 260269133 | Tissue Name | 260269083 | 260269132 | 260269133 |
| Colon cancer 1 | 0.0 | 0.0 | 3.5 | Bladder cancer | 0.0 | 0.0 | 0.0 |
| | | | | NAT 2 | | | |
| Colon cancer | 7.2 | 0.0 | 11.0 | Bladder cancer | 0.0 | 0.0 | 0.0 |
| NAT 1 | | | | NAT 3 | | | |
| Colon cancer 2 | 0.0 | 0.0 | 0.0 | Bladder cancer | 0.0 | 0.0 | 0.0 |
| | | | | NAT 4 | | | |
| Colon cancer | 17.6 | 16.6 | 15.7 | Prostate | 2.4 | 20.9 | 5.8 |
| NAT 2 | | | | adenocarcinoma 1 | | | |
| Colon cancer 3 | 4.5 | 0.0 | 3.8 | Prostate | 0.0 | 0.0 | 2.0 |
| | | | | adenocarcinoma 2 | | | |
| Colon cancer | 37.1 | 0.0 | 27.0 | Prostate | 71.7 | 55.9 | 54.3 |
| NAT3 | | | | adenocarcinoma 3 | | | |
| Colon | 6.1 | 0.0 | 1.0 | Prostate | 1.0 | 0.0 | 7.2 |
| malignant | | | | adenocarcinoma 4 | | | |
| cancer 4 | | | | | | | |
| Colon normal | 0.0 | 0.0 | 2.4 | Prostate cancer | 4.5 | 0.0 | 0.0 |
| adjacent tissue 4 | | | | NAT 5 | | | |
| Lung cancer 1 | 25.0 | 17.9 | 4.2 | Prostate | 30.6 | 4.5 | 11.1 |
| | | | | adenocarcinoma 6 | | | |
| Lung NAT 1 | 2.3 | 3.9 | 12.9 | Prostate | 14.4 | 6.3 | 23.0 |
| | | | | adenocarcinoma 7 | | | |
| Lung cancer 2 | 40.1 | 100.0 | 100.0 | Prostate | 9.1 | 5.0 | 6.8 |
| | | | | adenocarcinoma 8 | | | |
| Lung NAT 2 | 32.3 | 18.2 | 48.6 | Prostate | 75.3 | 10.7 | 31.0 |
| | | | | adenocarcinoma 9 | | | |
| Squamous cell | 73.2 | 47.0 | 82.4 | Prostate cancer | 0.0 | 0.0 | 7.1 |
| carcinoma 3 | | | | NAT 10 | | | |
| Lung NAT 3 | 13.3 | 3.5 | 5.8 | Kidney cancer 1 | 0.0 | 0.0 | 0.0 |
| metastatic | 4.4 | 0.0 | 1.5 | KidneyNAT 1 | 33.7 | 11.7 | 10.7 |
| melanoma 1 | | | | | | | |
| Melanoma 2 | 0.0 | 0.0 | 1.4 | Kidney cancer 2 | 10.7 | 7.4 | 2.8 |
| Melanoma 3 | 9.8 | 0.0 | 4.2 | Kidney NAT 2 | 100.0 | 42.9 | 51.4 |
| metastatic | 2.1 | 0.0 | 1.0 | Kidney cancer 3 | 61.1 | 8.6 | 24.8 |
| melanoma 4 | | | | | | | |
| metastatic | 6.4 | 9.3 | 2.2 | Kidney NAT 3 | 63.3 | 16.0 | 29.9 |
| melanoma 5 | | | | | | | |
| Bladder cancer 1 | 0.0 | 0.0 | 0.0 | Kidney cancer 4 | 8.8 | 0.0 | 1.9 |
| Bladder cancer | 0.0 | 0.0 | 0.0 | Kidney NAT 4 | 5.3 | 0.0 | 9.2 |
| NAT 1 | | | | | | | |
| Bladder cancer 2 | 2.1 | 0.0 | 0.0 | | | | |

AI_comprehensive panel_v1.0 Summary: Ag5242 Highest expression is seen in osteoarthritic bone sample (CT=27.5). Prominenet levels of expression are seen in a cluster of samples derived from RA. Thus, expression of this gene could be used to differentiate between these samples and other samples on this panel and as a marker of rheumatoid arthritis. In addition, modulation of the expression or function of this gene may be useful in the treatment of RA.

CNS_neurodegeneration_v1.0 Summary: Ag5242/Ag5243/Ag5247/Ag5248 Multiple experiments with four different probe and primer sets produce results that are in reasonable agreement. These panels do not show differential expression of this gene in Alzheimer's disease. However, these profiles confirm the expression of this gene at moderate levels in the brain. Please see Panel 1.5 for discussion of this gene in the central nervous system.

Ag5244 Three experiments with Ag5244, which is specific for CG150799-03, detect expression of this gene at low but significant levels in the hippocampus and temporal cortex of Alzheimer's patients. This expression may suggest an involvement of this gene product in the etiology of this disease.

One experiment with Ag5244 (Run 276863567) and two experiments with Ag5245 (Run 276863569 and Run 277731463), also specific for CG150799-03, show low/undetectable levels of expression (CTs>35). (Data not shown). Two additional experiments with Ag5245 show low expression in samples from the parietal cortex of a normal patient and the inferior temporal cortex of an Alzheimer's patient.

General_screen_panel_v1.5

Summary: Ag5242/Ag5243/Ag5245/Ag5247/Ag5248 Multiple experiments with five different probe and primer sets produce results that are in reasonable agreement. Highest expression is seen in cell lines from lung and prostate cancers and the fetal brain (CTs=28-30). This gene, which encodes a MASS 1 homolog, appears be preferentially expressed in the brain, with prominent levels of expression in all regions of the CNS examined. MASS 1 is a large, calcium-binding GPCR expressed in the central nervous system that may play a fundamental role in its development (MacMillan, J Biol Chem 2002 Jan 4;277(1):785-92). In addition, this gene has been associated with some nonsymptomatic epilepsies (Skardski, Neuron, Vol 31, 537-544, August 2001). Thus, based on the homology of this protein to MASSI and the preferential expression in the brain, expression of this gene could be used to differentiate between brain and non-neural tissue. In addition, therapeutic modulation of the expression or function of this gene may be useful in the treatment of neurological disorders, such as Alzheimer's disease, Parkinson's disease, schizophrenia, multiple sclerosis, stroke and epilepsy.

Moderate levels of expression are also seen in samples from lung, colon, ovarian and prostate cancer cell lines. This suggests that expression of this gene could be used as a marker of these cancers. Futhermore, therapeutic modulation of the expression or function of this gene may be useful in the treatment of these cancers.

Ag5244 Expression of this gene is low/undetectable (CTs>35) across all of the samples on this panel.

General_screen_panel_v1.6 Summary: Ag5243/Ag5247/Ag5248/Ag5245 Multiple experiments with three different probe and primer sets produce results that are in very good agreement. Highest expression is seen in a lung cancer cell line and the fetal brain (CTs=27-32). Overall, expression is in excellent agreement with Panel 1.5, with prominent expression seen in all regions of the CNS, and lung and prostate cancer cell lines. Please see Panel 1.5 for further discussion of this gene.

Ag5244 Expression of this gene is low/undetectable (CTs>35) across all of the samples on this panel.

Panel 4.1D Summary: Ag5242/Ag5243/Ag5247/Ag5248 Multiple experiments with four different probe and primers sets show highest expression of this gene in primary activated Th1 cells and resting neutrophils (CTs=27-31). Since this gene is expressed predominantly in activated Th-1 vs Th-2 cells, regulation of the expression of this gene might also be important for autoimmune disease such as rheumatoid arthritis (please see also AI panel). Moderate levels of expression are also seen in IL-4 treated lung fibroblasts and resting neutrophils. Thus, therapeutic regulation of the transcript or the protein encoded by the transcript could be important in immune modulation and in the treatment of T cell-mediated diseases such as asthma, arthritis, psoriasis, IBD, and lupus.

Ag5245 Highest expression of this gene is seen in IL-4 treated lung fibroblasts (CT=32). Low but significant expression is also seen in TNF-a/IL1-b treated lung fibroblasts and primary activated Th1 cells. Three experiments with the probe and primer set Ag5244 show low/undetectable levels of results (CTs>35).

General oncology screening panel_v_{—}2.4

Summary: Ag5242/Ag5243/Ag5247/Ag5248 Four experiments with the different probe and primer sets show highest expression in a lung cancers and normal kidney tissue adjacent to a tumor (CTs=31-34). Overall, this gene is expressed at low but significant levels in prostate cancer, normal kidney and kidney cancer, squamous cell carcinoma and normal colon. Therefore, therapeutic modulation of this gene or its protein product may be useful in the treatment of lung, prostate and kidney cancers.

Ag5244/Ag5245 Expression of this gene is low/undetectable in all samples on this panel (CTs>35).

AG. CG151014-01: Metabotropic Glutamate Receptor 3-Variant

Expression of gene CG151014-01 was assessed using the primer-probe set Ag5219, described in Table AGA. Results of the RTQ-PCR runs are shown in Tables AGB, AGC and AGD.

TABLE AGA

Probe Name Ag5219

| | | | Start | SEQ ID |
|---|---|---|---|---|
| Primers | | Length | Position | No |
| Forward | 5′-tgattgtgaattgcagttcagt-3′ | 22 | 2550 | 381 |
| Probe | TET-5′-aagtgctcacgtgcagctccagaata-3′-TAMRA | 26 | 2598 | 382 |
| Reverse | 5′-gtactagggttgttcttttgctct-3′ | 24 | 2631 | 383 |

TABLE AGB
CNS_neurodegeneration_v1.0

| | Rel. | | Rel. |
|---|---|---|---|
| | Exp. (%) | | Exp. (%) |
| | Ag5219, | | Ag5219, |
| | Run | | Run |
| Tissue Name | 228020421 | issue Name | 228020421 |
| AD 1 Hippo | 9.4 | Control (Path) 3 Temporal Ctx | 6.5 |
| AD 2 Hippo | 24.8 | Control (Path) 4 Temporal Ctx | 25.0 |
| AD 3 Hippo | 6.3 | AD 1 Occipital Ctx | 15.7 |
| AD 4 Hippo | 7.6 | AD 2 Occipital Ctx (Missing) | 0.0 |
| AD 5 Hippo | 53.2 | AD 3 Occipital Ctx | 6.8 |
| AD 6 Hippo | 24.1 | AD 4 Occipital Ctx | 33.2 |
| Control 2 Hippo | 40.9 | AD 5 Occipital Ctx | 51.8 |
| Control 4 Hippo | 6.7 | AD 6 Occipital Ctx | 15.3 |
| Control (Path) 3 Hippo | 5.6 | Control 1 Occipital Ctx | 7.6 |
| AD 1 Temporal Ctx | 19.1 | Control 2 Occipital Ctx | 46.0 |
| AD 2 Temporal Ctx | 34.9 | Control 3 Occipital Ctx | 16.6 |
| AD 3 Temporal Ctx | 5.6 | Control 4 Occipital Ctx | 8.5 |
| AD 4 Temporal Ctx | 25.3 | Control (Path) 1 Occipital Ctx | 90.1 |
| AD 5 Inf Temporal Ctx | 100.0 | Control (Path) 2 Occipital Ctx | 11.5 |
| AD 5 Sup Temporal Ctx | 32.5 | Control (Path) 3 Occipital Ctx | 3.8 |
| AD 6 Inf Temporal Ctx | 44.1 | Control (Path) 4 Occipital Ctx | 11.9 |
| AD 6 Sup Temporal Ctx | 32.5 | Control 1 Parietal Ctx | 9.5 |
| Control 1 Temporal Ctx | 10.5 | Control 2 Parietal Ctx | 40.6 |
| Control 2 Temporal Ctx | 45.4 | Control 3 Parietal Ctx | 18.3 |
| Control 3 Temporal Ctx | 28.9 | Control (Path) 1 Parietal Ctx | 74.2 |
| Control 3 Temporal Ctx | 10.1 | Control (Path) 2 Parietal Ctx | 27.5 |
| Control (Path) 1 Temporal Ctx | 65.1 | Control (Path) 3 Parietal Ctx | 5.0 |
| Control (Path) 2 Temporal Ctx | 36.1 | Control (Path) 4 Parietal Ctx | 36.3 |

TABLE AGC
General_screening_panel_v1.5

| | Rel. | | Rel. |
|---|---|---|---|
| | Exp. (%) | | Exp. (%) |
| | Ag5219, | | Ag5219, |
| | Run | | Run |
| Tissue Name | 228758224 | issue Name | 228758224 |
| Adipose | 0.3 | Renal ca. TK-10 | 0.4 |
| Melanoma* Hs688(A).T | 0.0 | Bladder | 0.2 |
| Melanoma* Hs688(B).T | 0.0 | Gastric ca. (liver met.) NCI-N87 | 6.6 |
| Melanoma* M14 | 0.0 | Gastric ca. KATO III | 0.0 |
| Melanoma* LOXIMVI | 0.5 | Colon ca. SW-948 | 0.1 |
| Melanoma* SK-MEL-5 | 0.8 | Colon ca. SW480 | 0.6 |
| Squamous cell carcinoma SCC-4 | 0.8 | Colon ca.* (SW480 met) SW620 | 1.1 |
| Testis Pool | 0.4 | Colon ca. HT29 | 0.0 |
| Prostate ca.* (bone met) PC-3 | 2.1 | Colon ca. HCT-116 | 1.7 |
| Prostate Pool | 0.5 | Colon ca. CaCo-2 | 0.7 |
| Placenta | 0.0 | Colon cancer tissue | 0.0 |
| Uterus Pool | 0.2 | Colon ca. SW1116 | 0.0 |
| Ovarian ca. OVCAR-3 | 1.0 | Colon ca. Colo-205 | 0.0 |
| Ovarian ca. SK-OV-3 | 0.9 | Colon ca. SW-48 | 0.0 |
| Ovarian ca. OVCAR-4 | 0.0 | Colon Pool | 0.7 |
| Ovarian ca. OVCAR-5 | 0.2 | Small Intestine Pool | 0.7 |
| Ovarian ca. IGROV-1 | 0.0 | Stomach Pool | 1.4 |
| Ovarian ca. OVCAR-8 | 0.1 | Bone Marrow Pool | 0.1 |
| Ovary | 0.1 | Fetal Heart | 0.6 |
| Breast ca. MCF-7 | 0.0 | Heart Pool | 0.3 |
| Breast ca. MDA-MB-231 | 0.5 | Lymph Node Pool | 1.1 |
| Breast ca. BT 549 | 0.0 | Fetal Skeletal Muscle | 0.1 |
| Breast ca. T47D | 0.0 | Skeletal Muscle Pool | 0.7 |
| Breast ca. MDA-N | 0.0 | Spleen Pool | 1.4 |
| Breast Pool | 2.6 | Thymus pool | 0.4 |
| Trachea | 0.4 | CNS cancer (glio/astro) U87-MG | 1.0 |
| Lung | 0.2 | CNS cancer (glio/astro) U-118-MG | 0.1 |
| Fetal Lung | 0.8 | CNS cancer (neuro; met) SK-N-AS | 1.4 |
| Lung ca. NCI-N417 | 0.1 | CNS cancer (astro) SF-539 | 0.0 |
| Lung ca. LX-1 | 4.5 | CNS cancer (astro) SNB-75 | 0.0 |
| Lung ca. NCI-H146 | 1.1 | CNS cancer (glio) SNB-19 | 0.0 |
| Lung ca. SHP-77 | 3.3 | CNS cancer (glio) SF-295 | 0.0 |
| Lung ca. A549 | 0.0 | Brain (Amygdala) Pool | 60.3 |
| Lung ca. NCI-H526 | 0.3 | Brain (cerebellum) | 100.0 |
| Lung ca. NCI-H23 | 0.4 | Brain (fetal) | 66.4 |
| Lung ca. NCI-H460 | 0.9 | Brain (Hippocampus) Pool | 43.5 |
| Lung ca. HOP-62 | 0.0 | Cerebral Cortex Pool | 80.1 |
| Lung ca. NCI-H522 | 0.7 | Brain (Substantia nigra) Pool | 54.0 |
| Liver | 0.0 | Brain (Thalamus) Pool | 94.6 |
| Fetal Liver | 0.4 | Brain (whole) | 65.1 |
| Liver ca. HepG2 | 0.9 | Spinal Cord Pool | 36.6 |
| Kidney Pool | 1.5 | Adrenal Gland | 0.6 |
| Fetal Kidney | 0.7 | Pituitary gland Pool | 0.9 |
| Renal ca. 786-0 | 0.0 | Salivary Gland | 0.2 |
| Renal ca. A498 | 0.0 | Thyroid (female) | 0.0 |
| Renal ca. ACHN | 1.0 | Pancreatic ca. CAPAN2 | 0.1 |
| Renal ca. UO-31 | 0.5 | Pancreas Pool | 0.9 |

TABLE AGD
Panel 4.1D

| | Rel. | | Rel. |
|---|---|---|---|
| | Exp (%) | | Exp. (%) |
| | Ag5219, | | Ag5219, |
| | Run | | Run |
| Tissue Name | 229739298 | Tissue Name | 229739298 |
| Secondary Th1 act | 0.0 | HUVEC IL-1beta | 3.3 |
| Secondary Th2 act | 3.2 | HUVEC IFN gamma | 14.0 |
| Secondary Tr1 act | 0.0 | HUVEC TNF alpha + IFN gamma | 2.9 |
| Secondary Th1 rest | 0.0 | HUVEC TNF alpha + IL4 | 1.8 |
| Secondary Th2 rest | 0.0 | HUVEC IL-11 | 21.8 |
| Secondary Tr1 rest | 2.9 | Lung Microvascular EC none | 100.0 |
| Primary Th1 act | 0.0 | Lung Microvascular EC TNF alpha + IL-1beta | 31.9 |
| Primary Th2 act | 5.8 | Microvascular Dermal EC none | 0.0 |
| Primary Tr1 act | 0.0 | Microsvasular Dermal EC | 15.5 |
| | | TNF alpha + IL-1beta | |
| Primary Th1 rest | 0.0 | Bronchial epithelium TNF alpha + IL1beta | 0.0 |
| Primary Th2 rest | 1.8 | Small airway epithelium none | 0.0 |
| Primary Tr1 rest | 4.7 | Small airway epithelium TNF alpha + IL-1beta | 3.4 |
| CD45RA CD4 lymphocyte act | 0.0 | Coronery artery SMC rest | 2.3 |
| CD45RO CD4 lymphocyte act | 11.1 | Coronery artery SMC TNF alpha + IL-1beta | 0.0 |
| CD8 lymphocyte act | 6.7 | Astrocytes rest | 0.0 |
| Secondary CD8 lymphocyte rest | 5.9 | Astrocytes TNF alpha + IL-1beta | 3.4 |
| Secondary CD8 lymphocyte act | 0.0 | KU-812 (Basophil) rest | 4.1 |
| CD4 lymphocyte none | 3.3 | KU-812 (Basophil) | 26.1 |
| | | PMA/ionomycin | |
| 2ry Th1/Th2/Tr1_anti-CD95 | 5.9 | CCD1106 (Keratinocytes) none | 4.5 |
| CH11 | | | |
| LAK cells rest | 3.0 | CCD1106 (Keratinocytes) | 0.0 |
| | | TNF alpha + IL-1beta | |
| LAK cells IL-2 | 2.0 | Liver cirrhosis | 0.0 |
| LAK cells IL-2 + IL-12 | 0.0 | NCI-H292 none | 18.2 |
| LAK cells IL-2 + IFN gamma | 3.0 | NCI-H292 IL-4 | 16.7 |
| LAK cells IL-2 + IL-18 | 2.7 | NCI-H292 IL-9 | 25.0 |
| LAK cells PMA/ionomycin | 0.0 | NCI-H292 IL-13 | 48.3 |
| NK Cells IL-2 rest | 24.1 | NCI-H292 IFN gamma | 19.9 |
| Two Way MLR 3 day | 3.5 | HPAEC none | 8.1 |
| Two Way MLR 5 day | 1.5 | HPAEC TNF alpha + IL-1beta | 7.8 |
| Two Way MLR 7 day | 0.0 | Lung fibroblast none | 0.0 |
| PBMC rest | 0.0 | Lung fibroblast TNF alpha + IL-1 | 2.0 |
| | | beta | |
| PBMC PWM | 1.0 | Lung fibroblast IL-4 | 7.9 |
| PBMC PHA-L | 0.0 | Lung fibroblast IL-9 | 0.0 |
| Ramos (B cell) none | 18.2 | Lung fibroblast IL-13 | 0.0 |
| Ramos (B cell) ionomycin | 59.9 | Lung fibroblast IFN gamma | 2.8 |
| B lymphocytes PWM | 4.2 | Dermal fibroblast CCD1070 rest | 0.0 |
| B lymphocytes CD40L and IL-4 | 13.2 | Dermal fibroblast CCD1070 TNF | 0.0 |
| | | alpha | |
| EOL-1 dbcAMP | 0.0 | Dermal fibroblast CCD1070 IL-1 | 6.7 |
| | | beta | |
| EOL-1 dbcAMP | 4.8 | Dermal fibroblast IFN gamma | 40.6 |
| PMA/ionomycin | | | |
| Dendritic cells none | 4.4 | Dermal fibroblast IL-4 | 25.0 |
| Dendritic cells LPS | 0.0 | Dermal fibroblasts rest | 2.1 |
| Dendritic cells anti-CD40 | 0.0 | Neutrophils TNFa + LPS | 0.0 |
| Monocytes rest | 0.0 | Neutrophils rest | 0.0 |
| Monocytes LPS | 0.0 | Colon | 0.0 |
| Macrophages rest | 0.0 | Lung | 0.0 |
| Macrophages LPS | 0.0 | Thymus | 0.0 |
| HUVEC none | 1.7 | Kidney | 11.3 |
| HUVEC starved | 28.1 | | |

CNS_neurodegeneration_v1.0 Summary: Ag5219 This panel confirms the expression of this gene at low levels in the brain in an independent group of individuals. This gene is found to be slightly down-regulated in the temporal cortex of Alzheimer's disease patients. Therefore, up-regulation of this gene or its protein product, or treatment with specific agonists for this receptor may be of use in reversing the dementia, memory loss, and neuronal death associated with this disease.

General_screening₁₃ panel_v1.5 Summary: Ag5219 Highest expression of this gene is deted in cerebellum (CT=27). High expression of this gene is mainly seen in all the region of central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

In addition, moderate to low levels of expression of this gene is also seen in a number of cancer cell lines derived from brain, colon, gastric, lung, ovarian, and prostate cancers, squamous cell carcinoma and melanoma. Therefore, therapeutic modulation of this gene may be useful in the treatment of these cancers.

Low levels of expression of this gene is also seen in tissues with metabolic/endocrine functions including pancreas, adrenal and pituitary cancers, fetal heart, skeletal muscle and gastrointestinal tract. Therefore, therapeutic modulation of the activity of this gene may prove useful in the treatment of endocrine/metabolically related diseases, such as obesity and diabetes.

Panel 4.1D Summary: Ag5219 Highest expression of this gene is detected in lung microvascular endothelial cells (CT=32.4). This gene is expressed at lower levels in cytokine activated lung microvascular cells, activated dermal fibroblasts, resting and activated mucoepidermoid NCI-H292, activated basophils, starved and I-11 stimulated HVEC cells, Ramos B cells, and resting IL-2 treated NK cells. Therefore, therapeutic modulation of this gene may be useful in the treatment of autoimmune and inflammatory diseases such as asthma, allergies, inflammatory bowel disease, lupus erythematosus, psoriasis, rheumatoid arthritis, and osteoarthritis.

AH. CG151014-02 and CG151014-03: Metabotropic Glutamate Receptor 3.

Expression of gene CG151014-02 and CG151014-02 was assessed using the primer-probe set Ag5220, described in Table AHA. Results of the RTQ-PCR runs are shown in Tables AHB and AHC. Please note that CG151014-03 represents a full-length physical clone.

TABLE AHA

Probe Name Ag5220

| | | | Start | SEQ ID |
|---|---|---|---|---|
| Primers | | Length | Position | No |
| Forward | 5′-atcaacttcacgggtgcag-3′ | 19 | 1399 | 384 |
| Probe | TET-5′-ctttgtggtcttgggctgtttgtttg-3′-TAMRA | 26 | 1453 | 385 |
| Reverse | 5′-caggatgatgtgaaccttgg-3′ | 20 | 1482 | 386 |

TABLE AHB
CNS_neurodegeneration_v1.0

| | Rel. | | Rel. |
|---|---|---|---|
| | Exp. (%) | | Exp. (%) |
| | Ag5220, | | Ag5220, |
| | Run | | Run |
| Tissue Name | 228020422 | issue Name | 228020422 |
| AD 1 Hippo | 2.0 | Control (Path) 3 Temporal Ctx | 5.8 |
| AD 2 Hippo | 49.0 | Control (Path) 4 Temporal Ctx | 25.2 |
| AD 3 Hippo | 1.0 | AD 1 Occipital Ctx | 5.6 |
| AD 4 Hippo | 13.5 | AD 2 Occipital Ctx (Missing) | 0.0 |
| AD 5 hippo | 35.4 | AD 3 Occipital Ctx | 3.1 |
| AD 6 Hippo | 59.9 | AD 4 Occipital Ctx | 24.7 |
| Control 2 Hippo | 34.2 | AD 5 Occipital Ctx | 17.2 |
| Control 4 Hippo | 7.0 | AD 6 Occipital Ctx | 61.6 |
| Control (Path) 3 Hippo | 4.4 | Control 1 Occipital Ctx | 2.6 |
| AD 1 Temporal Ctx | 6.0 | Control 2 Occipital Ctx | 43.2 |
| AD 2 Temporal Ctx | 39.2 | Control 3 Occipital Ctx | 10.2 |
| AD 3 Temporal Ctx | 2.4 | Control 4 Occipital Ctx | 9.0 |
| AD 4 Temporal Ctx | 29.9 | Control (Path) 1 Occipital Ctx | 100.0 |
| AD 5 Inf Temporal Ctx | 76.3 | Control (Path) 2 Occipital Ctx | 7.7 |
| AD 5 SupTemporal Ctx | 29.9 | Control (Path) 3 Occipital Ctx | 2.1 |
| AD 6 Inf Temporal Ctx | 60.3 | Control (Path) 4 Occipital Ctx | 14.2 |
| AD 6 Sup Temporal Ctx | 69.3 | Control 1 Parietal Ctx | 7.0 |
| Control 1 Temporal Ctx | 13.2 | Control 2 Parietal Ctx | 24.3 |
| Control 2 Temporal Ctx | 52.9 | Control 3 Parietal Ctx | 15.4 |
| Control 3 Temporal Ctx | 23.3 | Control (Path) 1 Parietal Ctx | 89.5 |
| Control 4 Temporal Ctx | 11.7 | Control (Path) 2 Parietal Ctx | 15.2 |
| Control (Path) 1 Temporal Ctx | 87.1 | Control (Path) 3 Parietal Ctx | 6.4 |
| Control (Path) 2 Temporal Ctx | 59.0 | Control (Path) 4 Parietal Ctx | 33.0 |

TABLE AHC
General_screening_panel_v1.5

| | Rel. | | Rel. |
|---|---|---|---|
| | Exp. (%) | | Exp. (%) |
| | Ag5220, | | Ag5220, |
| | Run | | Run |
| Tissue Name | 228758228 | issue Name | 228758228 |
| Adipose | 0.0 | Renal ca. TK-10 | 0.0 |
| Melanoma* Hs688(A).T | 0.0 | Bladder | 0.0 |
| Melanoma* Hs688(B).T | 0.0 | Gastric ca. (liver met.) NCI-N87 | 0.0 |
| Melanoma* M14 | 0.0 | Gastric ca. KATO III | 0.0 |
| Melanoma* LOXIMVI | 0.0 | Colon ca. SW-948 | 0.0 |
| Melanoma* SK-MEL-5 | 0.0 | Colon ca. SW480 | 0.0 |
| Squamous cell carcinoma SCC-4 | 0.0 | Colon ca.* (SW480 met) SW620 | 0.0 |
| Testis Pool | 0.0 | Colon ca. HT29 | 0.0 |
| Prostate ca.* (bone met) PC-3 | 0.0 | Colon ca. HCT-116 | 0.0 |
| Prostate Pool | 0.0 | Colon ca. CaCo-2 | 0.0 |
| Placenta | 0.0 | Colon cancer tissue | 0.0 |
| Uterus Pool | 0.0 | Colon ca. SW1116 | 0.0 |
| Ovarian ca. OVCAR-3 | 0.0 | Colon ca. Colo-205 | 0.0 |
| Ovarian ca. SK-OV-3 | 0.0 | Colon ca. SW-48 | 0.0 |
| Ovarian ca. OVCAR-4 | 0.0 | Colon Pool | 0.0 |
| Ovarian ca. OVCAR-5 | 0.0 | Small Intestine Pool | 0.0 |
| Ovarian ca. IGROV-1 | 0.0 | Stomach Pool | 1.6 |
| Ovarian ca. OVCAR-8 | 0.0 | Bone Marrow Pool | 0.0 |
| Ovary | 0.0 | Fetal Heart | 0.0 |
| Breast ca. MCF-7 | 0.0 | Heart Pool | 0.0 |
| Breast ca. MDA-MB-231 | 0.0 | Lymph Node Pool | 0.7 |
| Breast ca. BT 549 | 0.0 | Fetal Skeletal Muscle | 0.0 |
| Breast ca. T47D | 0.0 | Skeletal Muscle Pool | 0.0 |
| Breast ca. MDA-N | 0.0 | Spleen Pool | 0.0 |
| Breast Pool | 2.3 | Thymus Pool | 0.0 |
| Trachea | 0.0 | CNS cancer (glio/astro) U87-MG | 0.0 |
| Lung | 0.0 | CNS cancer (glio/astro) U-118-MG | 0.0 |
| Fetal Lung | 0.0 | CNS cancer (neuro; met) SK-N-AS | 0.0 |
| Lung ca. NCI-N417 | 0.0 | CNS cancer (astro) SF-539 | 0.0 |
| Lung ca. LX-1 | 0.0 | CNS cancer (astro) SNB-75 | 0.0 |
| Lung ca. NCI-H146 | 0.0 | CNS cancer (glio) SNB-19 | 0.0 |
| Lung ca. SHP-77 | 0.0 | CNS cancer (glio) SF-295 | 0.0 |
| Lung ca. A549 | 0.0 | Brain (Amygdala) Pool | 75.8 |
| Lung ca. NCI-H526 | 0.0 | Brain (cerebellum) | 100.0 |
| Lung ca. NCI-H23 | 0.0 | Brain (fetal) | 69.3 |
| Lung ca. NCI-H460 | 0.2 | Brain (Hippocampus) Pool | 53.2 |
| Lung ca. HOP-62 | 0.0 | Cerebral Cortex Pool | 72.2 |
| Lung ca. NCI-H522 | 0.0 | Brain (Substantia nigra) Pool | 80.7 |
| Liver | 0.0 | Brain (Thalamus) Pool | 96.6 |
| Fetal Liver | 0.0 | Brain (whole) | 78.5 |
| Liver ca. HepG2 | 0.0 | Spinal Cord Pool | 25.0 |
| Kidney Pool | 0.0 | Adrenal Gland | 4.3 |
| Fetal Kidney | 0.5 | Pituitary gland Pool | 0.0 |
| Renal ca. 786-0 | 0.0 | Salivary Gland | 0.0 |
| Renal ca. A498 | 0.0 | Thyroid (female) | 0.0 |
| Renal ca. ACHN | 0.0 | Pancreatic ca. CAPAN2 | 0.0 |
| Renal ca. UO-31 | 0.0 | Pancreas Pool | 0.0 |

CNS_neurodegeneration_v1.0 Summary: Ag5220 This panel confirms the expression of this gene at low levels in the brains of an independent group of individuals. However, no differential expression of this gene was detected between Alzheimer's diseased postmortem brains and those of non-demented controls in this experiment. Please see Panel 1.5 for a discussion of this gene in treatment of central nervous system disorders.

General_screen_panel_v1.5 Summary: Ag5220 Highest expression of this gene is deted in cerebellum (CT=27). High expression of this gene is mainly seen in all the region of central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

Panel 4.1D Summary: Ag5220 Expression of this gene is low/undetectable (CTs>35) across all of the samples on this panel.

AI. CG151297-01: Calmodulin-Dependent Phosphodiesterase.

Expression of gene CG151297-01 was assessed using the primer-probe set Ag7165, described in Table AIA. Results of the RTQ-PCR runs are shown in Table AIB. Please note that CG151297-01 represents a full-length physical clone.

TABLE AIA

Probe Name Ag7165

| | | | Start | SEQ ID |
|---|---|---|---|---|
| Primers | Sequences | Length | Position | No |
| Forward | 5′-agaatgtaccgaaaaacattttctct-3′ | 26 | 481 | 387 |
| Probe | TET-5′-ttcctcttatagaggaagcctcaaaagccg-3′-TAMRA | 30 | 536 | 388 |
| Reverse | 5′-tgcttgccacataggaagaa-3′ | 20 | 570 | 389 |

TABLE AIB
Panel 4.1D

| | Rel. | | Rel. |
|---|---|---|---|
| | Exp. (%) | | Exp. (%) |
| | Ag7165, | | Ag7165, |
| | Run | | Run |
| Tissue Name | 307719896 | Tissue Name | 307719896 |
| Secondary Th1 act | 0.0 | HUVEC IL-1beta | 0.0 |
| Secondary Th2 act | 0.0 | HUVEC IFN gamma | 0.0 |
| Secondary Tr1 act | 0.0 | HUVEC TNF alpha + IFN gamma | 0.0 |
| Secondary Th1 rest | 0.0 | HUVEC TNF alpha + IL4 | 0.0 |
| Secondary Th2 rest | 0.0 | HUVEC IL-11 | 0.0 |
| Secondary Tr1 rest | 0.0 | Lung Microvascular EC none | 0.0 |
| Primary Th1 act | 0.0 | Lung Microvascular EC TNF alpha + IL-1beta | 0.0 |
| Primary Th2 act | 0.0 | Microvascular Dermal EC none | 0.0 |
| Primary Tr1 act | 0.0 | Microsvasular Dermal EC | 0.0 |
| | | TNF alpha + IL-1beta | |
| Primary Th1 rest | 0.0 | Bronchial epithelium TNF alpha + IL1beta | 0.0 |
| Primary Th2 rest | 0.0 | Small airway epithelium none | 0.0 |
| Primary Tr1 rest | 0.0 | Small airway epithelium TNF alpha + IL-1beta | 0.0 |
| CD45RA CD4 lymphocyte act | 0.0 | Coronery artery SMC rest | 0.0 |
| CD45RO CD4 lymphocyte act | 0.0 | Coronery artery SMC TNF alpha +IL-1beta | 0.0 |
| CD8 lymphocyte act | 0.0 | Astrocytes rest | 0.0 |
| Secondary CD8 lymphocyte rest | 0.0 | Astrocytes TNF alpha + IL-1beta | 0.0 |
| Secondary CD8 lymphocyte act | 0.0 | KU-812 (Basophil) rest | 0.0 |
| CD4 lymphocyte none | 0.0 | KU-812 (Basophil) | 0.0 |
| | | PMA/ionomycin | |
| 2ry Th1/Th2/Tr1_anti-CD95 | 0.0 | CCD1106 (Keratinocytes) none | 0.0 |
| CH11 | | | |
| LAK cells rest | 0.0 | CCD1106 (Keratinocytes) | 0.0 |
| | | TNF alpha + IL-1beta | |
| LAK cells IL-2 | 0.0 | Liver cirrhosis | 100.0 |
| LAK cells IL-2 + IL-12 | 0.0 | NCI-H292 none | 0.0 |
| LAK cells IL-2 + IFN gamma | 0.0 | NCI-H292 IL-4 | 0.0 |
| LAK cells IL-2 + IL-18 | 0.0 | NCI-H292 IL-9 | 0.0 |
| LAK cells PMA/ionomycin | 0.0 | NCI-H292 IL-13 | 0.0 |
| NK Cells IL-2 rest | 0.0 | NCI-H292 IFN gamma | 0.0 |
| Two Way MLR 3 day | 0.0 | HPAEC none | 0.0 |
| Two Way MLR 5 day | 0.0 | HPAEC TNF alpha + IL-1beta | 0.0 |
| Two Way MLR 7 day | 0.0 | Lung fibroblast none | 0.0 |
| PBMC rest | 0.0 | Lung fibroblast TNF alpha + IL-1 | 0.0 |
| | | beta | |
| PBMC PWM | 0.0 | Lung fibroblast IL-4 | 0.0 |
| PBMC PHA-L | 0.0 | Lung fibroblast IL-9 | 0.0 |
| Ramos (B cell) none | 0.0 | Lung fibroblast IL-13 | 0.0 |
| Ramos (B cell) ionomycin | 0.0 | Lung fibroblast IFN gamma | 0.0 |
| B lymphocytes PWM | 0.0 | Dermal fibroblast CCD1070 rest | 0.0 |
| B lymphocytes CD40L and IL-4 | 0.0 | Dermal fibroblast CCD1070 TNF | 0.0 |
| | | alpha | |
| EOL-1 dbcAMP | 0.0 | Dermal fibroblast CCD1070 IL-1 | 0.0 |
| | | beta | |
| EOL-1 dbcAMP | 0.0 | Dermal fibroblast IFN gamma | 0.0 |
| PMA/ionomycin | | | |
| Dendritic cells none | 0.0 | Dermal fibroblast IL-4 | 0.0 |
| Dendritic cells LPS | 0.0 | Dermal Fibroblasts rest | 0.0 |
| Dendritic cells anti-CD40 | 0.0 | Neutrophils TNFa + LPS | 0.0 |
| Monocytes rest | 0.0 | Neutrophils rest | 0.0 |
| Monocytes LPS | 0.0 | Colon | 0.0 |
| Macrophages rest | 0.0 | Lung | 0.0 |
| Macrophages LPS | 0.0 | Thymus | 0.0 |
| HUVEC none | 0.0 | Kidney | 0.0 |
| HUVEC starved | 0.0 | | |

CNS_neurodegeneration_v1.0 Summary: Ag7165 Expression of this gene is low/undetectable (CTs>35) across all of the samples on this panel.

Panel 4.1D Summary: Ag7165 Moderate level of expression of this gene is detected mainly in the liver cirrhosis sample (CT=31.5). The presence of this gene in liver cirrhosis (a component of which involves liver inflammation and fibrosis) suggests that antibodies to the protein encoded by this gene could also be used for the diagnosis of liver cirrhosis. Furthermore, therapeutic agents involving this gene may be useful in reducing or inhibiting the inflammation associated with fibrotic and inflammatory diseases.

AJ. CG152256-01: Phosphatidylserine Synthase.

Expression of gene CG152256-01 was assessed using the primer-probe set Ag6718, described in Table AJA. Results of the RTQ-PCR runs are shown in Tables AJB, AJC and AJD.

TABLE AJA

Probe Name Ag6718

| | | | Start | SEQ ID |
|---|---|---|---|---|
| Primers | | Length | Position | No |
| Forward | 5′-gagcctcgcttccgattat-3′ | 19 | 2012 | 390 |
| Probe | TET-5′-tcccttcccaatattattcatccaga-3′-TAMRA | 26 | 2031 | 391 |
| Reverse | 5′-ctctagcaggtttgcttttgtg-3′ | 22 | 2070 | 392 |

TABLE AJB
CNS_neurodegeneration_v1.0

| | Rel. | | Rel. |
|---|---|---|---|
| | Exp. (%) | | Exp. (%) |
| | Ag6718, | | Ag6718, |
| | Run | | Run |
| Tissue Name | 276596848 | issue Name | 276596848 |
| AD 1 Hippo | 19.8 | Control (Path) 3 Temporal Ctx | 2.6 |
| AD 2 Hippo | 26.6 | Control (Path) 4 Temporal Ctx | 15.3 |
| AD 3 Hippo | 4.3 | AD 1 Occipital Ctx | 9.9 |
| AD 4 Hippo | 3.7 | AD 2 Occipital Ctx (Missing) | 0.0 |
| AD 5 Hippo | 58.6 | AD 3 Occipital Ctx | 7.1 |
| AD 6 Hippo | 45.4 | AD 4 Occipital Ctx | 15.9 |
| Control 2 Hippo | 28.5 | AD 5 Occipital Ctx | 26.6 |
| Control 4 Hippo | 8.4 | AD 6 Occipital Ctx | 15.1 |
| Control (Path) 3 Hippo | 3.1 | Control 1 Occipital Ctx | 3.6 |
| AD 1 Temporal Ctx | 4.8 | Control 2 Occipital Ctx | 67.4 |
| AD 2 Temporal Ctx | 24.7 | Control 3 Occipital Ctx | 31.2 |
| AD 3 Temporal Ctx | 7.5 | Control 4 Occipital Ctx | 1.8 |
| AD 4 Temporal Ctx | 10.5 | Control (Path) 1 Occipital Ctx | 100.0 |
| AD 5 Inf Temporal Ctx | 62.9 | Control (Path) 2 Occipital Ctx | 9.5 |
| AD 5 Sup Temporal Ctx | 46.3 | Control (Path) 3 Occipital Ctx | 5.3 |
| AD 6 Inf Temporal Ctx | 43.5 | Control (Path) 4 Occipital Ctx | 10.0 |
| AD6 Sup Temporal Ctx | 43.2 | Control 1 Parietal Ctx | 3.8 |
| Control 1 Temporal Ctx | 4.1 | Control 2 Parietal Ctx | 27.9 |
| Control 2 Temporal Ctx | 59.0 | Control 3 Parietal Ctx | 15.0 |
| Control 3 Temporal Ctx | 17.6 | Control (Path) 1 Parietal Ctx | 89.5 |
| Control 3 Temporal Ctx | 5.0 | Control (Path) 2 Parietal Ctx | 10.2 |
| Control (Path) 1 Temporal Ctx | 57.0 | Control (Path) 3 Parietal Ctx | 7.0 |
| Control (Path) 2 Temporal Ctx | 30.4 | Control (Path) 4 Parietal Ctx | 27.9 |

TABLE AJC
General_screening_panel v1.6

| | Rel. | | Rel. |
|---|---|---|---|
| | Exp. (%) | | Exp. (%) |
| | Ag6718, | | Ag6718, |
| | Run | | Run |
| Tissue Name | 277223813 | issue Name | 277223813 |
| Adipose | 2.3 | Renal ca. TK-10 | 34.4 |
| Melanoma* Hs688(A).T | 16.4 | Bladder | 22.2 |
| Melanoma* Hs688(B).T | 20.0 | Gastric ca. (liver met.) NCI-N87 | 54.0 |
| Melanoma* M14 | 30.6 | Gastric ca. KATO III | 48.3 |
| Melanoma* LOXIMVI | 55.1 | Colon ca. SW-948 | 31.0 |
| Melanoma* SK-MEL-5 | 81.8 | Colon ca. SW480 | 87.1 |
| Squamous cell carcinoma SCC-4 | 23.5 | Colon ca.* (SW480 met) SW620 | 69.7 |
| Testis Pool | 5.2 | Colon ca. HT29 | 0.0 |
| Prostate ca.* (bone met) PC-3 | 100.0 | Colon ca. HCT-116 | 51.4 |
| Prostate Pool | 1.8 | Colon ca. CaCo-2 | 15.9 |
| Placenta | 2.6 | Colon cancer tissue | 23.5 |
| Uterus Pool | 0.8 | Colon ca. SW1116 | 25.0 |
| Ovarian ca. OVCAR-3 | 27.4 | Colon ca. Colo-205 | 21.9 |
| Ovarian ca. SK-OV-3 | 29.9 | Colon ca. SW-48 | 24.1 |
| Ovarian ca. OVCAR-4 | 33.0 | Colon Pool | 12.4 |
| Ovarian ca. OVCAR-5 | 59.9 | Small Intestine Pool | 4.8 |
| Ovarian ca. IGROV-1 | 47.6 | Stomach Pool | 1.8 |
| Ovarian ca. OVCAR-8 | 32.8 | Bone Marrow Pool | 0.0 |
| Ovary | 11.7 | Fetal Heart | 14.2 |
| Breast ca. MCF-7 | 18.9 | Heart Pool | 11.6 |
| Breast ca. MDA-MB-231 | 48.0 | Lymph Node Pool | 3.8 |
| Breast ca. BT 549 | 31.6 | Fetal Skeletal Muscle | 3.3 |
| Breast ca. T47D | 3.6 | Skeletal Muscle Pool | 0.0 |
| Breast ca. MDA-N | 17.9 | Spleen Pool | 2.0 |
| Breast Pool | 7.0 | Thymus pool | 11.7 |
| Trachea | 9.2 | CNS cancer (glio/astro) U87-MG | 32.3 |
| Lung | 2.4 | CNS cancer (glio/astro) U-118-MG | 43.2 |
| Fetal Lung | 4.9 | CNS cancer (neuro; met) SK-N-AS | 25.9 |
| Lung ca. NCI-N417 | 15.0 | CNS cancer (astro) SF-539 | 29.5 |
| Lung ca. LX-1 | 17.6 | CNS cancer (astro) SNB-75 | 59.0 |
| Lung ca. NCI-H146 | 23.7 | CNS cancer (glio) SNB-19 | 29.7 |
| Lung ca. SHP-77 | 53.2 | CNS cancer (glio) SF-295 | 59.5 |
| Lung ca. A549 | 28.3 | Brain (Amygdala) Pool | 10.4 |
| Lung ca. NCI-H526 | 24.3 | Brain (cerebellum) | 34.4 |
| Lung ca. NCI-H23 | 71.7 | Brain (fetal) | 17.3 |
| Lung ca. NCI-H460 | 14.2 | Brain (Hippocampus) Pool | 9.4 |
| Lung ca. HOP-62 | 32.3 | Cerebral Cortex Pool | 7.4 |
| Lung ca. NCI-H522 | 16.4 | Brain (Substantia nigra) Pool | 3.9 |
| Liver | 1.0 | Brain (Thalamus) Pool | 6.9 |
| Fetal Liver | 2.3 | Brain (whole) | 6.5 |
| Liver ca. HepG2 | 19.2 | Spinal Cord Pool | 5.6 |
| Kidney Pool | 15.2 | Adrenal Gland | 10.3 |
| Fetal Kidney | 4.1 | Pituitary gland Pool | 1.1 |
| Renal ca. 786-0 | 61.6 | Salivary Gland | 3.2 |
| Renal ca. A498 | 5.6 | Thyroid (female) | 11.5 |
| Renal ca. ACHN | 24.7 | Pancreatic ca. CAPAN2 | 28.1 |
| Renal ca. UO-31 | 33.9 | Pancreas Pool | 8.3 |

TABLE AJD
Panel 4.1D

| | Rel. | | Rel. |
|---|---|---|---|
| | Exp. (%) | | Exp. (%) |
| | Ag6718, | | Ag6718, |
| | Run | | Run |
| Tissue Name | 276596888 | Tissue Name | 276596888 |
| Secondary Th1 act | 51.4 | HUVEC IL-1beta | 18.0 |
| Secondary Th2 act | 39.5 | HUVEC IFN gamma | 16.5 |
| Secondary Tr1 act | 19.3 | HUVEC TNF alpha + IFN gamma | 4.5 |
| Secondary Th1 rest | 5.3 | HUVEC TNF alpha + IL4 | 3.1 |
| Secondary Th2 rest | 4.5 | HUVEC IL-11 | 0.0 |
| Secondary Tr1 rest | 5.9 | Lung Microvascular EC none | 13.9 |
| Primary Th1 act | 3.5 | Lung Microvascular EC TNF alpha + IL-1beta | 0.7 |
| Primary Th2 act | 20.7 | Microvascular Dermal EC none | 3.0 |
| Primary Tr1 act | 12.8 | Microsvasular Dermal EC | 1.2 |
| | | TNF alpha + IL-1beta | |
| Primary Th1 rest | 1.6 | Bronchial epithelium TNF alpha + IL1beta | 5.8 |
| Primary Th2 rest | 5.8 | Small airway epithelium none | 6.3 |
| Primary Tr1 rest | 0.7 | Small airway epithelium TNF alpha + IL-1beta | 9.7 |
| CD45RA CD4 lymphocyte act | 26.4 | Coronery artery SMC rest | 7.1 |
| CD45RO CD4 lymphocyte act | 30.8 | Coronery artery SMC TNF alpha + IL-1beta | 8.4 |
| CD8 lymphocyte act | 7.6 | Astrocytes rest | 3.3 |
| Secondary CD8 lymphocyte rest | 6.3 | Astrocytes TNF alpha + IL-1beta | 2.9 |
| Secondary CD8 lymphocyte act | 1.5 | KU-812 (Basophil) rest | 44.8 |
| CD4 lymphocyte none | 3.6 | KU-812 (Basophil) | 28.1 |
| | | PMA/ionomycin | |
| 2ry Th1/Th2/Tr1_anti-CD95 | 2.9 | CCD1106 (Keratinocytes) none | 27.5 |
| CH11 | | | |
| LAK cells rest | 4.5 | CCD1106 (Keratinocytes) | 5.1 |
| | | TNF alpha + IL-1beta | |
| LAK cells IL-2 | 9.9 | Liver cirrhosis | 0.8 |
| LAK cells IL-2 + IL-12 | 0.7 | NCI-H292 none | 8.0 |
| LAK cells IL-2 + IFN gamma | 4.2 | NCI-H292 IL-4 | 10.2 |
| LAK cells IL-2 + IL-18 | 1.4 | NCI-H292 IL-9 | 19.2 |
| LAK cells PMA/ionomycin | 18.7 | NCI-H292 IL-13 | 14.8 |
| NK Cells IL-2 rest | 21.0 | NCI-H292 IFN gamma | 6.8 |
| Two Way MLR 3 day | 7.6 | HPAEC none | 3.7 |
| Two Way MLR 5 day | 5.2 | HPAEC TNF alpha + IL-1beta | 8.5 |
| Two Way MLR 7 day | 4.3 | Lung fibroblast none | 6.8 |
| PBMC rest | 1.4 | Lung fibroblast TNF alpha + IL-1 | 1.9 |
| | | beta | |
| PBMC PWM | 3.0 | Lung fibroblast IL-4 | 6.1 |
| PBMC PHA-L | 4.1 | Lung fibroblast IL-9 | 10.0 |
| Ramos (B cell) none | 42.9 | Lung fibroblast IL-13 | 7.7 |
| Ramos (B cell) ionomycin | 22.1 | Lung fibroblast IFN gamma | 16.4 |
| B lymphocytes PWM | 10.8 | Dermal fibroblast CCD1070 rest | 33.9 |
| B lymphocytes CD40L and IL-4 | 12.2 | Dermal fibroblast CCD1070 TNF | 100.0 |
| | | alpha | |
| EOL-1 dbcAMP | 39.0 | Dermal fibroblast CCD1070 IL-1 | 17.4 |
| | | beta | |
| EOL-1 dbcAMP | 14.1 | Dermal fibroblast IFN gamma | 6.7 |
| PMA/ionomycin | | | |
| Dendritic cells none | 13.5 | Dermal fibroblast IL-4 | 10.4 |
| Dendritic cells LPS | 2.5 | Dermal Fibroblasts rest | 6.9 |
| Dendritic cells anti-CD40 | 4.5 | Neutrophils TNFa + LPS | 0.4 |
| Monocytes rest | 0.6 | Neutrophils rest | 0.7 |
| Monocytes LPS | 3.9 | Colon | 0.8 |
| Macrophages rest | 1.4 | Lung | 0.6 |
| Macrophages LPS | 3.8 | Thymus | 2.9 |
| HUVEC none | 11.1 | Kidney | 8.1 |
| HUVEC starved | 6.4 | | |

CNS_neurodegeneration_v1.0 Summary: Ag6718 This panel confirms the expression of this gene at low levels in the brains of an independent group of individuals. However, no differential expression of this gene was detected between Alzheimer's diseased postmortem brains and those of non-demented controls in this experiment. Please see Panel 1.6 for a discussion of this gene in treatment of central nervous system disorders.

General_screen_panel_v1.6 Summary: Ag6718 Highest expression of this gene is detected in prostate cancer PC3 cell line (CT=31.9). Moderate levels of expression of this gene is also seen in cluster of cancer cell lines derived from pancreatic, gastric, colon, lung, liver, renal, breast, ovarian, prostate, squamous cell carcinoma, melanoma and brain cancers. Thus, expression of this gene could be used as a marker to detect the presence of these cancers. Furthermore, therapeutic modulation of the expression or function of this gene may be effective in the treatment of pancreatic, gastric, colon, lung, liver, renal, breast, ovarian, prostate, squamous cell carcinoma, melanoma and brain cancers.

In addition, this gene is expressed at low levels in cerebellum and fetal brain. Therefore, therapeutic modulation of this gene product may be useful in the treatment of central nervous system disorders such as ataxia and autism.

Panel 4.1D Summary: Ag6718 Highest expression of this gene is detected in TNF alpha treated dermal fibroblasts (CT=32). Moderate to low levels of expression of this gene is detected in activated polarized, naive and memory T cells, PMA/ionomycin treated LAK cells, resting IL-2 treated NK cells, Ramos B cells, cosinophils, activated HUVEC cells, lung microvascular endothelial cells, basophils and activated mucoepidermoid NCI-H292 cells. Therefore, therapeutic modulation of this gene or its protein product may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

AK. CG173017-01: Retinoic Acid Receptor Rxe-Beta.

Expression of gene CG173017-01 was assessed using the primer-probe set Ag7565, described in Table AKA.

TABLE AKA

Probe Name Ag7565

| | | | Start | SEQ ID |
|---|---|---|---|---|
| Primers | | Length | Position | No |
| Forward | 5′-ctggacgggacgggat-3′ | 16 | 222 | 393 |
| Probe | TET-5′-acatagccgtttgccagccccag-3′-TAMRA | 23 | 261 | 394 |
| Reverse | 5′-cttctgtccccgcagatt-3′ | 18 | 286 | 395 |

CNS_neurodegeneration_v1.0 Summary: Ag7565 Expression of this gene is low/undetectable in all samples on this panel (CTs>35).

Panel 4.1D Summary: Ag7565 Expression of this gene is low/undetectable in all samples on this panel (CTs>35).

AL. CG173347-01: Novel Serum Paraoxonase/arylesterase 3.

Expression of gene CG173347-01 was assessed using the primer-probe set Ag7564, described in Table ALA.

TABLE ALA

Probe Name Ag7564

| | | | Start | SEQ ID |
|---|---|---|---|---|
| Primers | Sequences | Length | Position | No |
| Forward | 5′-gaaagtggctctgaagatattgatatact-3′ | 29 | 153 | 396 |
| Probe | TET-5′-tcctagtgggctggcttttatctcc-3′-TAMRA | 25 | 182 | 397 |
| Reverse | 5′-actccaacagacctgcagact-3′ | 21 | 207 | 398 |

CNS_neurodegeneration_v1.0 Summary: Ag7564 Expression of this gene is low/undetectable in all samples on this panel (CTs>35).

Panel 4.1D Summary: Ag7564 Expression of this gene is low/undetectable in all samples on this panel (CTs>35).

AM. CG56234-02: Splice Variant of PCK2.

Expression of gene CG56234-02 was assessed using the primer-probe set Ag5111, described in Table AMA. Results of the RTQ-PCR runs are shown in Tables AMB, AMC, AMD and AME.

TABLE AMA

Probe Name Ag5111

| | | | Start | SEQ ID |
|---|---|---|---|---|
| Primers | | Length | Position | No |
| Forward | 5′-ctgggaggccccaga-3′ | 15 | 1377 | 399 |
| Probe | TET-5′-tgtccccattgacgccatcatc-3′-TAMRA | 22 | 1395 | 400 |
| Reverse | 5′-gatgatcttccctttgggtct-3′ | 21 | 1429 | 401 |

TABLE AMB
General_screening_panel_v1.5

| | Rel. | | Rel. |
|---|---|---|---|
| | Exp. (%) | | Exp. (%) |
| | Ag5111, | | Ag5111, |
| | Run | | Run |
| Tissue Name | 228980587 | issue Name | 228980587 |
| Adipose | 2.0 | Renal ca. TK-10 | 29.1 |
| Melanoma* Hs688(A).T | 31.9 | Bladder | 12.1 |
| Melanoma* Hs688(B).T | 28.3 | Gastric ca. (liver met.) NCI-N87 | 31.4 |
| Melanoma* M14 | 9.9 | Gastric ca. KATO III | 28.1 |
| Melanoma* LOXIMVI | 4.5 | Colon ca. SW-948 | 17.9 |
| Melanoma* SK-MEL-5 | 39.8 | Colon ca. SW480 | 14.9 |
| Squamous cell carcinoma SCC-4 | 4.7 | Colon ca.* (SW480 met) SW620 | 29.5 |
| Testis Pool | 1.6 | Colon ca. HT29 | 8.6 |
| Prostate ca.* (bone met) PC-3 | 55.1 | Colon ca. HCT-116 | 11.0 |
| Prostate Pool | 0.5 | Colon ca. CaCo-2 | 44.4 |
| Placenta | 0.3 | Colon cancer tissue | 9.7 |
| Uterus Pool | 0.6 | Colon ca. SW1116 | 1.4 |
| Ovarian ca. OVCAR-3 | 13.6 | Colon ca. Colo-205 | 6.6 |
| Ovarian ca. SK-OV-3 | 5.3 | Colon ca. SW-48 | 14.4 |
| Ovarian ca. OVCAR-4 | 7.1 | Colon Pool | 0.1 |
| Ovarian ca. OVCAR-5 | 34.6 | Small Intestine Pool | 0.6 |
| Ovarian ca. IGROV-1 | 22.5 | Stomach Pool | 1.1 |
| Ovarian ca. OVCAR-8 | 100.0 | Bone Marrow Pool | 0.5 |
| Ovary | 0.0 | Fetal Heart | 0.0 |
| Breast ca. MCF-7 | 87.7 | Heart Pool | 0.0 |
| Breast ca. MDA-MB-231 | 12.6 | Lymph Node Pool | 0.8 |
| Breast ca. BT 549 | 75.8 | Fetal Skeletal Muscle | 0.6 |
| Breast ca. T47D | 10.1 | Skeletal Muscle Pool | 0.4 |
| Breast ca. MDA-N | 16.4 | Spleen Pool | 1.7 |
| Breast Pool | 0.5 | Thymus Pool | 0.4 |
| Trachea | 4.3 | CNS cancer (glio/astro) U87-MG | 18.8 |
| Lung | 0.0 | CNS cancer (glio/astro) U-118-MG | 9.3 |
| Fetal Lung | 2.0 | CNS cancer (neuro; met) SK-N-AS | 7.5 |
| Lung ca. NCI-N417 | 1.8 | CNS cancer (astro) SF-539 | 11.3 |
| Lung ca. LX-1 | 8.2 | CNS cancer (astro) SNB-75 | 48.6 |
| Lung ca. NCI-H146 | 11.1 | CNS cancer (glio) SNB-19 | 31.0 |
| Lung ca. SHP-77 | 11.3 | CNS cancer (glio) SF-295 | 32.5 |
| Lung ca. A549 | 11.4 | Brain (Amygdala) Pool | 0.4 |
| Lung ca. NCI-H526 | 1.8 | Brain (cerebellum) | 0.3 |
| Lung ca. NCI-H23 | 83.5 | Brain (fetal) | 0.3 |
| Lung ca. NCI-H460 | 27.0 | Brain (Hippocampus) Pool | 2.5 |
| Lung ca. HOP-62 | 1.0 | Cerebral Cortex Pool | 0.4 |
| Lung ca. NCI-H522 | 67.4 | Brain (Substantia nigra) Pool | 0.0 |
| Liver | 6.3 | Brain (Thalamus) Pool | 1.0 |
| Fetal Liver | 6.7 | Brain (whole) | 0.7 |
| Liver ca. HepG2 | 24.7 | Spinal Cord Pool | 1.1 |
| Kidney Pool | 0.8 | Adrenal Gland | 1.6 |
| Fetal Kidney | 1.0 | Pituitary gland Pool | 0.4 |
| Renal ca. 786-0 | 8.7 | Salivary Gland | 0.9 |
| Renal ca. A498 | 1.5 | Thyroid (female) | 0.7 |
| Renal ca. ACHN | 9.3 | Pancreatic ca. CAPAN2 | 12.8 |
| Renal ca. UO-31 | 1.9 | Pancreas Pool | 0.8 |

TABLE AMC
General_screening_panel_v1.6

| | Rel. | Rel. | Rel. | | Rel. | Rel. | Rel. |
|---|---|---|---|---|---|---|---|
| | Exp. (%) | Exp. (%) | Exp. () | | Exp. (%) | Exp. (%) | Exp. (%) |
| | Ag5111, | Ag5111, | Ag5111, | | Ag5111, | Ag5111, | Ag5111, |
| | Run | Run | Run | | Run | Run | Run |
| Tissue Name | 277218717 | 277731246 | 278368614 | Tissue Name | 277218717 | 277731246 | 278368614 |
| Adipose | 0.5 | 0.0 | 1.5 | Renal ca. | 24.7 | 20.2 | 33.0 |
| | | | | TK-10 | | | |
| Melanoma* | 26.1 | 29.5 | 31.6 | Bladder | 6.7 | 6.1 | 11.6 |
| Hs688(A).T | | | | | | | |
| Melanoma* | 25.2 | 32.1 | 31.9 | Gastric ca. | 21.3 | 22.5 | 36.1 |
| Hs688(B).T | | | | (liver met.) | | | |
| | | | | NCI-N87 | | | |
| Melanoma* | 5.6 | 9.7 | 7.5 | Gastric ca. | 14.6 | 12.2 | 19.2 |
| M14 | | | | KATO III | | | |
| Melanoma* | 3.0 | 0.0 | 4.2 | Colon ca. | 18.8 | 16.5 | 23.5 |
| LOXIMVI | | | | SW-948 | | | |
| Melanoma* | 28.7 | 57.0 | 39.8 | Colon ca. | 11.8 | 7.3 | 19.5 |
| SK-MEL-5 | | | | SW480 | | | |
| Squamous cell | 4.8 | 4.2 | 5.1 | Colon ca.* | 23.0 | 19.9 | 35.6 |
| carcinoma | | | | (SW480 met) | | | |
| SCC-4 | | | | SW620 | | | |
| Testis Pool | 2.0 | 0.0 | 1.4 | Colon ca. | 10.2 | 4.2 | 8.2 |
| | | | | HT29 | | | |
| Prostate ca.* | 33.2 | 44.4 | 57.8 | Colon ca. | 9.6 | 7.6 | 19.9 |
| (bone met) | | | | HCT-116 | | | |
| PC-3 | | | | | | | |
| Prostate Pool | 0.3 | 0.0 | 0.6 | Colon ca. | 9.4 | 25.0 | 36.9 |
| | | | | CaCo-2 | | | |
| Placenta | 0.3 | 0.0 | 1.1 | Colon cancer | 6.0 | 0.0 | 6.6 |
| | | | | tissue | | | |
| Uterus Pool | 0.0 | 0.0 | 0.6 | Colon ca. | 2.3 | 0.0 | 1.7 |
| | | | | SW1116 | | | |
| Ovarian ca. | 12.7 | 8.2 | 18.2 | Colon ca. | 5.1 | 4.7 | 5.9 |
| OVCAR-3 | | | | Colo-205 | | | |
| Ovarian ca. | 5.3 | 6.5 | 12.2 | Colon ca. | 9.0 | 0.0 | 11.6 |
| SK-OV-3 | | | | SW-48 | | | |
| Ovarian ca. | 4.0 | 5.2 | 5.8 | Colon Pool | 0.7 | 0.0 | 0.7 |
| OVCAR-4 | | | | | | | |
| Ovarian ca. | 31.6 | 24.8 | 34.2 | Small Intestine | 0.3 | 0.0 | 0.8 |
| OVCAR-5 | | | | Pool | | | |
| Ovarian ca. | 19.2 | 12.8 | 27.2 | Stomach Pool | 1.2 | 0.0 | 2.3 |
| IGROV- 1 | | | | | | | |
| Ovarian ca. | 100.0 | 100.0 | 100.0 | Bone Marrow | 0.0 | 0.0 | 0.0 |
| OVCAR-8 | | | | Pool | | | |
| Ovary | 0.0 | 0.0 | 0.2 | Fetal Heart | 0.0 | 0.0 | 0.3 |
| Breast ca. | 54.0 | 51.4 | 77.9 | Heart Pool | 0.4 | 0.0 | 0.0 |
| MCF-7 | | | | | | | |
| Breast ca. | 8.5 | 7.6 | 7.7 | Lymph Node | 1.2 | 0.0 | 0.0 |
| MDA-MB-231 | | | | Pool | | | |
| Breast ca. BT | 47.0 | 30.4 | 49.0 | Fetal Skeletal | 0.0 | 0.0 | 0.0 |
| 549 | | | | Muscle | | | |
| Breast ca. T47D | 5.1 | 6.5 | 7.1 | Skeletal | 0.0 | 0.0 | 0.0 |
| | | | | Muscle Pool | | | |
| Breast ca. | 6.1 | 6.0 | 24.5 | Spleen Pool | 0.7 | 0.0 | 2.5 |
| MDA-N | | | | | | | |
| Breast Pool | 0.3 | 0.0 | 0.3 | Thymus Pool | 0.5 | 0.0 | 1.8 |
| Trachea | 3.3 | 0.0 | 8.3 | CNS cancer | 12.9 | 7.9 | 13.8 |
| | | | | (glio/astro) | | | |
| | | | | U87-MG | | | |
| Lung | 0.0 | 0.0 | 0.0 | CNS cancer | 5.9 | 4.4 | 8.1 |
| | | | | (glio/astro) | | | |
| | | | | U-118-MG | | | |
| Fetal Lung | 0.9 | 0.0 | 2.1 | CNS cancer | 6.4 | 4.9 | 6.7 |
| | | | | (neuro; met) | | | |
| | | | | SK-N-AS | | | |
| Lung ca. | 1.3 | 0.0 | 3.8 | CNS cancer | 5.8 | 6.4 | 8.5 |
| NCI-N417 | | | | (astro) SF-539 | | | |
| Lung ca. LX-1 | 5.5 | 7.8 | 9.5 | CNS cancer | 25.0 | 29.9 | 26.8 |
| | | | | (astro) | | | |
| | | | | SNB-75 | | | |
| Lung ca. | 8.0 | 8.5 | 11.5 | CNS cancer | 23.8 | 20.7 | 29.5 |
| NCI-H146 | | | | (glio) SNB-19 | | | |
| Lung ca. | 12.2 | 14.3 | 21.3 | CNS cancer | 38.2 | 28.7 | 46.7 |
| SHP-77 | | | | (glio) SF-295 | | | |
| Lung ca. A549 | 11.5 | 11.7 | 15.9 | Brain | 0.8 | 0.0 | 1.1 |
| | | | | (Amygdala) | | | |
| | | | | Pool | | | |
| Lung ca. | 1.8 | 0.0 | 1.7 | Brain | 1.0 | 0.0 | 1.1 |
| NCI-H526 | | | | (cerebellum) | | | |
| Lung ca. | 42.6 | 68.8 | 55.1 | Brain (fetal) | 0.0 | 0.0 | 0.4 |
| NCI-H23 | | | | | | | |
| Lung ca. | 16.7 | 23.5 | 38.4 | Brain | 0.4 | 0.0 | 1.2 |
| NCI-H460 | | | | (Hippocampus) | | | |
| | | | | Pool | | | |
| Lung ca. | 2.0 | 0.0 | 3.0 | Cerebral | 0.0 | 0.0 | 0.6 |
| HOP-62 | | | | Cortex Pool | | | |
| Lung ca. | 41.5 | 64.2 | 87.1 | Brain | 0.0 | 0.0 | 0.4 |
| NCI-H522 | | | | (Substantia | | | |
| | | | | nigra) Pool | | | |
| Liver | 4.4 | 4.6 | 7.1 | Brain | 0.0 | 0.0 | 0.0 |
| | | | | (Thalamus) | | | |
| | | | | Pool | | | |
| Fetal Liver | 5.8 | 3.3 | 8.7 | Brain (whole) | 6.7 | 0.0 | 2.8 |
| Liver ca. | 15.7 | 16.3 | 18.8 | Spinal Cord | 0.6 | 0.0 | 0.5 |
| HepG2 | | | | Pool | | | |
| Kidney Pool | 0.7 | 0.0 | 0.3 | Adrenal Gland | 1.4 | 0.0 | 1.4 |
| Fetal Kidney | 0.9 | 0.0 | 1.0 | Pituitary gland | 0.0 | 0.0 | 0.7 |
| | | | | Pool | | | |
| Renal ca. 786-0 | 9.3 | 8.1 | 13.8 | Salivary Gland | 0.8 | 0.0 | 1.8 |
| Renal ca. A498 | 1.1 | 0.0 | 2.0 | Thyroid | 1.0 | 0.0 | 2.1 |
| | | | | (female) | | | |
| Renal ca. | 5.8 | 6.0 | 10.8 | Pancreatic ca. | 13.1 | 9.6 | 19.9 |
| ACHN | | | | CAPAN2 | | | |
| Renal ca. | 2.4 | 0.0 | 3.3 | Pancreas Pool | 4.8 | 0.0 | 7.3 |
| UO-31 | | | | | | | |

TABLE AMD
Panel 4.1D

| | Rel. | Rel. | | Rel. | Rel. |
|---|---|---|---|---|---|
| | Exp. (%) | Exp. (%) | | Exp. (%) | Exp. (%) |
| | g5111, | Ag5111, | | Ag5111, | Ag5111, |
| | Run | Run | | Run | Run |
| Tissue Name | 226444761 | 276596864 | Tissue Name | 226444761 | 276596864 |
| Secondary Th1 act | 90.8 | 58.6 | HUVEC IL-1beta | 18.7 | 10.7 |
| Secondary Th2 act | 40.9 | 57.8 | HUVEC IFN gamma | 2.8 | 6.2 |
| Secondary Tr1 act | 57.4 | 16.5 | HUVEC TNF alpha + IFN | 5.0 | 6.2 |
| | | | gamma | | |
| Secondary Th1 rest | 27.2 | 8.4 | HUVEC TNF alpha + IL4 | 23.2 | 8.8 |
| Secondary Th2 rest | 6.0 | 0.0 | HUVEC IL-11 | 2.3 | 0.0 |
| Secondary Tr1 rest | 7.2 | 4.0 | Lung Microvascular | 3.2 | 15.4 |
| | | | EC none | | |
| Primary Th1 act | 32.8 | 5.0 | Lung Microvascular | 6.4 | 0.0 |
| | | | EC TNF alpha + IL-1beta | | |
| Primary Th2 act | 49.0 | 19.9 | Microvascular | 6.6 | 0.0 |
| | | | Dermal EC none | | |
| Primary Tr1 act | 50.0 | 38.4 | Microsvasular | 0.0 | 0.0 |
| | | | Dermal EC | | |
| | | | TNF alpha + IL-1beta | | |
| Primary Th1 rest | 6.0 | 8.5 | Bronchial epithelium | 8.7 | 6.9 |
| | | | TNF alpha + IL1beta | | |
| Primary Th2 rest | 6.4 | 6.3 | Small airway | 2.2 | 0.0 |
| | | | epithelium none | | |
| Primary Tr1 rest | 18.0 | 0.0 | Small airway | 11.8 | 0.0 |
| | | | epithelium TNF alpha + | | |
| | | | IL-1beta | | |
| CD45RA CD4 | 95.9 | 76.8 | Coronery artery SMC | 18.3 | 10.2 |
| lymphocyte act | | | rest | | |
| CD45RO CD4 | 95.3 | 100.0 | Coronery artery SMC | 9.4 | 8.8 |
| lymphocyte act | | | TNF alpha + IL-1beta | | |
| CD8 lymphocyte act | 77.4 | 4.5 | Astrocytes rest | 2.1 | 0.0 |
| Secondary CD8 | 90.1 | 17.3 | Astrocytes TNF alpha + | 0.0 | 0.0 |
| lymphocyte rest | | | IL-1beta | | |
| Secondary CD8 | 21.0 | 7.7 | KU-812 (Basophil) | 25.9 | 10.2 |
| lymphocyte act | | | rest | | |
| CD4 lymphocyte none | 0.0 | 0.0 | KU-812 (Basophil) | 26.8 | 21.2 |
| | | | PMA/ionomycin | | |
| 2ry | 5.4 | 0.0 | CCD1106 | 15.2 | 4.9 |
| Th1/Th2/Tr1_anti-CD95 | | | (Keratinocytes) none | | |
| CH11 | | | | | |
| LAK cells rest | 43.5 | 19.9 | CCD1106 | 9.0 | 12.3 |
| | | | (Keratinocytes) | | |
| | | | TNF alpha + IL-1beta | | |
| LAK cells IL-2 | 52.1 | 18.4 | Liver cirrhosis | 8.3 | 0.0 |
| LAK cells IL-2 + IL-12 | 33.7 | 0.0 | NCI-H292 none | 15.3 | 3.4 |
| LAK cells IL-2 + IFN | 57.0 | 6.6 | NCI-H292 IL-4 | 13.5 | 17.2 |
| gamma | | | | | |
| LAK cells IL-2 + IL-18 | 46.0 | 9.5 | NCI-H292 IL-9 | 14.2 | 14.1 |
| LAK cells | 43.5 | 24.5 | NCI-H292 IL-13 | 29.1 | 11.3 |
| PMA/ionomycin | | | | | |
| NK Cells IL-2 rest | 60.7 | 37.4 | NCI-H292 IFN | 44.8 | 7.2 |
| | | | gamma | | |
| Two Way MLR 3 day | 32.1 | 10.3 | HPAEC none | 2.0 | 0.0 |
| Two Way MLR 5 day | 53.2 | 3.6 | HPAEC TNF alpha + IL-1 | 7.2 | 7.0 |
| | | | beta | | |
| Two Way MLR 7 day | 23.5 | 9.6 | Lung fibroblast none | 21.2 | 15.9 |
| PBMC rest | 6.1 | 0.0 | Lung fibroblast TNF | 11.5 | 0.0 |
| | | | alpha + IL-1beta | | |
| PBMC PWM | 23.5 | 9.1 | Lung fibroblast IL-4 | 2.4 | 0.0 |
| PBMC PHA-L | 35.8 | 12.2 | Lung fibroblast IL-9 | 17.6 | 5.4 |
| Ramos (B cell) none | 58.6 | 16.7 | Lung fibroblast IL-13 | 13.4 | 0.0 |
| Ramos (B cell) | 71.7 | 92.7 | Lung fibroblast IFN | 11.6 | 3.1 |
| ionomycin | | | gamma | | |
| B lymphocytes PWM | 21.6 | 14.8 | Dermal fibroblast | 99.3 | 64.6 |
| | | | CCD1070 rest | | |
| B lymphocytes CD40L | 29.7 | 23.2 | Dermal fibroblast | 74.7 | 88.9 |
| and IL-4 | | | CCD1070 TNF alpha | | |
| EOL-1 dbcAMP | 32.3 | 32.8 | Dermal fibroblast | 29.9 | 50.0 |
| | | | CCD1070 IL-1beta | | |
| EOL-1 dbcAMP | 10.6 | 3.2 | Dermal fibroblast | 13.3 | 0.0 |
| PMA/ionomycin | | | IFN gamma | | |
| Dendritic cells none | 66.0 | 24.5 | Dermal fibroblast | 12.2 | 0.0 |
| | | | IL-4 | | |
| Dendritic cells LPS | 31.4 | 0.0 | Dermal Fibroblasts | 0.0 | 0.0 |
| | | | rest | | |
| Dendritic cells | 48.3 | 28.1 | Neutrophils | 0.0 | 0.0 |
| anti-CD40 | | | TNFa + LPS | | |
| Monocytes rest | 29.1 | 0.0 | Neutrophils rest | 0.0 | 0.0 |
| Monocytes LPS | 37.6 | 18.0 | Colon | 32.3 | 8.2 |
| Macrophages rest | 100.0 | 12.9 | Lung | 3.5 | 0.0 |
| Macrophages LPS | 28.1 | 16.2 | Thymus | 12.1 | 0.0 |
| HUVEC none | 7.9 | 5.7 | Kidney | 83.5 | 31.9 |
| HUVEC starved | 17.4 | 8.4 | | | |

TABLE AME
general oncology screening panel_v_2.4

| | Rel. | | Rel. |
|---|---|---|---|
| | Exp. (%) | | Exp. (%) |
| | Ag5111, | | Ag5111, |
| | Run | | Run |
| Tissue Name | 260280403 | Tissue Name | 260280403 |
| Colon cancer 1 | 49.0 | Bladder cancer NAT 2 | 0.0 |
| Colon cancer NAT 1 | 2.5 | Bladder cancer NAT 3 | 0.0 |
| Colon cancer 2 | 11.7 | Bladder cancer NAT 4 | 0.0 |
| Colon cancer NAT 2 | 28.5 | Prostate adenocarcinoma 1 | 5.0 |
| Colon cancer 3 | 43.5 | Prostate adenocarcinoma 2 | 0.0 |
| Colon cancer NAT 3 | 53.2 | Prostate adenocarcinoma 3 | 0.0 |
| Colon malignant cancer 4 | 100.0 | Prostate adenocarcinoma 4 | 0.0 |
| Colon normal adjacent tissue 4 | 8.4 | Prostate cancer NAT 5 | 0.0 |
| Lung cancer 1 | 12.2 | Prostate adenocarcinoma 6 | 0.0 |
| Lung NAT 1 | 0.0 | Prostate adenocarcinoma 7 | 0.0 |
| Lung cancer 2 | 72.2 | Prostate adenocarcinoma 8 | 0.0 |
| Lung NAT 2 | 0.0 | Prostate adenocarcinoma 9 | 4.0 |
| Squamous cell carcinoma 3 | 18.8 | Prostate cancer NAT 10 | 0.0 |
| Lung NAT 3 | 0.0 | Kidney cancer 1 | 7.5 |
| metastatic melanoma 1 | 0.0 | KidneyNAT 1 | 0.0 |
| Melanoma 2 | 6.3 | Kidney cancer 2 | 73.2 |
| Melanoma 3 | 0.0 | Kidney NAT 2 | 9.2 |
| metastatic melanoma 4 | 0.0 | Kidney cancer 3 | 6.3 |
| metastatic melanoma 5 | 2.0 | Kidney NAT 3 | 0.0 |
| Bladder cancer 1 | 0.0 | Kidney cancer 4 | 7.6 |
| Bladder cancer NAT 1 | 0.0 | Kidney NAT 4 | 84.1 |
| Bladder cancer 2 | 0.0 | | |

CNS_neurodegeneration_v1.0 Summary: Ag5111 Expression of the CG56234-02 gene is low/undetectable in all samples on this panel (CTs>35).

General_screen_panel_v1.5 Summary: Ag5111 Highest expression of the CG56234-02 gene is seen in an ovarian cancer cell line (CT=30). This gene encodes a splice variant of PEPCK2, the rate-limiting enzyme for gluconeogenesis that has been shown to be regulated in response to hormones and environmental stress. In addition, to the ovarian cancer cell line, this gene is expressed at a moderate level in most of the cancer cell lines used in this panel. Therefore, modulation of the gene product using small molecule drugs may affect the growth and survival of cancer cells. Expression of this gene could potentially be used as a diagnostic marker of the metabolic status of cells and inhibition of activity of this gene prodcut might be used for therapeutic treatment of cancers.

This gene is also moderately expressed (CT values=34) in adult and fetal liver. Inhibition of this enzyme could potentially decrease hepatic glucose production and thus serve as an effective treatment for Type 2 diabetes, which is characterized by excess hepatic glucose production.

General_screen_panel_v1.6 Summary: Ag5111 Three experiments with the same probe and primer produce results that are in excellent agreement. Highest expression is seen in an ovarian cancer cell line (CTs=31-34) and overall, expression of this gene appears to be more highly associated with cancer cell line samples than with normal tissue samples. These results are also in agreement with results in Panel 1.5. Please see that panel for discussion of this gene.

Panel 4.1D Summary: Ag5111 This gene is expressed at low levels in a wide range of cell across this panel (CTs=33.3-34.4), including CD4 T cells (naive and memory T cells), CD8 T cells, B cells and macrophages. Expression of this transcript is also found in dermal fibroblasts and kidney. This transcript encodes a homolog of a key enzyme in glucogenesis and therefore may be important for the metabolic status of all these cell types which contribute to the inflammatory response. Therefore, modulation of the activity or expression of this putative protein by small molecules could affect the activity of these cells and be useful for the treatment of autoimmune diseases such as inflammatory bowel diseases, rheumatoid arthritis, asthma, COPD, psoriasis and lupus.

General oncology screening panel_v_{—}2.4 Summary: Ag5111 Low but significant expression is seen in a colon cancer, a kidney cancer, and a lung cancer (CTs=34-35). This is in agreement with the preferential expression in cancer cell lines seen in Panels 1.5 and 1.6. Please see Panel 1.5 for discussion of this gene in oncology.

AN. CG56836-03: Cathepsin B.

Expression of gene CG56836-03 was assessed using the primer-probe sets Ag2052 and Ag5278, described in Tables ANA, ANB and ANC. Results of the RTQ-PCR runs are shown in Tables AND, ANE, ANF, ANG, ANH, ANI, ANJ and ANK.

TABLE ANA

Probe Name Ag2052

| | | | Start | SEQ ID |
|---|---|---|---|---|
| Primers | | Length | Position | No |
| Forward | 5′-gtcccaccatcaaagagatca-3′ | 21 | 414 | 402 |
| Probe | TET-5′-agaccagggctcctgtggctcct-3′-TAMRA | 23 | 436 | 403 |
| Reverse | 5′-atgcagatccggtcagagat-3′ | 20 | 485 | 404 |

TABLE ANB

Probe Name Ag5277

| | | | Start | SEQ ID |
|---|---|---|---|---|
| Primers | | Length | Position | No |
| Forward | 5′-gatctgcatccacaccaat-3′ | 19 | 390 | 405 |
| Probe | TET-5′-cctgctcacctgcctgctctacaagt-3′-TAMRA | 26 | 441 | 406 |
| Reverse | 5′-cagtcagtgttccaggagtt-3′ | 20 | 568 | 407 |

TABLE ANC

Probe Name Ag5278

| | | | Start | SEQ ID |
|---|---|---|---|---|
| Primers | | Length | Position | No |
| Forward | 5′-tatgaatccaatagcgaga-3′ | 19 | 653 | 408 |
| Probe | TET-5′-agctttctctgtgtattcggacttcc-3′-TAMRA | 26 | 715 | 409 |
| Reverse | 5′-tgttggtacactcctgactt-3′ | 20 | 749 | 410 |

TABLE AND
AI_comprehensive panel_v1.0

| | Rel. | | Rel. |
|---|---|---|---|
| | Exp. (%) | | Exp. (%) |
| | Ag2052, | | Ag2052, |
| | Run | | Run |
| Tissue Name | 275804031 | issue Name | 275804031 |
| 110967 COPD-F | 10.2 | 112427 Match Control Psoriasis-F | 15.4 |
| 110980 COPD-F | 6.4 | 112418 Psoriasis-M | 10.4 |
| 110968 COPD-M | 12.0 | 112723 Match Control Psoriasis-M | 5.9 |
| 110977 COPD-M | 14.0 | 112419 Psoriasis-M | 12.9 |
| 110989 Emphysema-F | 15.6 | 112424 Match Control Psoriasis-M | 4.3 |
| 110992 Emphysema-F | 20.0 | 112420 Psoriasis-M | 29.7 |
| 110993 Emphysema-F | 13.8 | 112425 Match Control Psoriasis-M | 14.8 |
| 110994 Emphysema-F | 6.0 | 104689 (MF) OA Bone-Backus | 29.9 |
| 110995 Emphysema-F | 33.2 | 104690 (MF) Adj “Normal” | 15.4 |
| | | Bone-Backus | |
| 110996 Emphysema-F | 8.5 | 104691 (MF) OA Synovium-Backus | 55.9 |
| 110997 Asthma-M | 6.1 | 104692 (BA) OA Cartilage-Backus | 27.9 |
| 111001 Asthma-F | 6.7 | 104694 (BA) OA Bone-Backus | 39.5 |
| 111002 Asthma-F | 11.2 | 104695 (BA) Adj “Normal” | 23.0 |
| | | Bone-Backus | |
| 111003 Atopic Asthma-F | 9.7 | 104696 (BA) OA Synovium-Backus | 100.0 |
| 111004 Atopic Asthma-F | 12.2 | 104700 (SS) OA Bone-Backus | 12.2 |
| 111005 Atopic Asthma-F | 7.4 | 104701 (SS) Adj “Normal” | 24.3 |
| | | Bone-Backus | |
| 111006 Atopic Asthma-F | 1.7 | 104702 (SS) OA Synovium-Backus | 43.8 |
| 111417 Allergy-M | 9.0 | 117093 OA Cartilage Rep7 | 18.4 |
| 112347 Allergy-M | 0.0 | 112672 OA Bone5 | 17.3 |
| 112349 Normal Lung-F | 0.0 | 112673 OA Synovium5 | 6.6 |
| 112357 Normal Lung-F | 10.7 | 112674 OA Synovial Fluid cells5 | 8.4 |
| 112354 Normal Lung-M | 3.6 | 117100 OA Cartilage Rep14 | 8.4 |
| 112374 Crohns-F | 10.6 | 112756 OA Bone9 | 13.4 |
| 112389 Match Control Crohns-F | 14.1 | 112757 OA Synovium9 | 4.0 |
| 112375 Crohns-F | 9.9 | 112758 OA Synovial Fluid Cells9 | 5.0 |
| 112732 Match Control Crohns-F | 6.6 | 117125 RA Cartilage Rep2 | 19.5 |
| 112725 Crohns-M | 1.3 | 113492 Bone2 RA | 11.7 |
| 112387 Match Control | 11.7 | 113493 Synovium2 RA | 3.6 |
| Crohns-M | | | |
| 112378 Crohns-M | 0.0 | 113494 Syn Fluid Cells RA | 6.7 |
| 112390 Match Control | 14.5 | 113499 Cartilage4 RA | 6.7 |
| Crohns-M | | | |
| 112726 Crohns-M | 11.5 | 113500 Bone4 RA | 6.3 |
| 112731 Match Control | 7.5 | 113501 Synovium4 RA | 5.1 |
| Crohns-M | | | |
| 112380 Ulcer Col-F | 8.7 | 113502 Syn Fluid Cells4 RA | 3.4 |
| 112734 Match Control Ulcer | 15.4 | 113495 Cartilage3 RA | 7.2 |
| Col-F | | | |
| 112384 Ulcer Col-F | 25.7 | 113496 Bone3 RA | 7.0 |
| 112737 Match Control Ulcer | 4.1 | 113497 Synovium3 RA | 4.4 |
| Col-F | | | |
| 112386 Ulcer Col-F | 7.1 | 113498 Syn Fluid Cells3 RA | 9.7 |
| 112738 Match Control Ulcer | 13.1 | 117106 Normal Cartilage Rep20 | 8.1 |
| Col-F | | | |
| 112381 Ulcer Col-M | 0.1 | 113663 Bone3 Normal | 0.0 |
| 112735 Match Control Ulcer | 0.4 | 113664 Synovium3 Normal | 0.0 |
| Col-M | | | |
| 112382 Ulcer Col-M | 12.9 | 113665 Syn Fluid Cells3 Normal | 0.0 |
| 112394 Match Control Ulcer | 3.3 | 117107 Normal Cartilage Rep22 | 3.2 |
| Col-M | | | |
| 112383 Ulcer Col-M | 30.4 | 113667 Bone4 Normal | 6.3 |
| 112736 Match Control Ulcer | 11.0 | 113668 Synovium4 Normal | 8.1 |
| Col-M | | | |
| 112423 Psoriasis-F | 5.5 | 113669 Syn Fluid Cells4 Normal | 12.9 |

TABLE ANE
General_screening_panel_v1.5

| | Rel. | | Rel. |
|---|---|---|---|
| | Exp. (%) | | Exp. (%) |
| | Ag5278, | | Ag5278, |
| | Run | | Run |
| Tissue Name | 230509757 | issue Name | 230509757 |
| Adipose | 0.2 | Renal ca. TK-10 | 6.2 |
| Melanoma* Hs688(A).T | 24.0 | Bladder | 5.1 |
| Melanoma* Hs688(B).T | 12.9 | Gastric ca. (liver met.) NCI-N87 | 9.7 |
| Melanoma* M14 | 51.8 | Gastric ca. KATO III | 5.7 |
| Melanoma* LOXIMVI | 26.6 | Colon ca. SW-948 | 2.1 |
| Melanoma* SK-MEL-5 | 17.0 | Colon ca. SW480 | 7.0 |
| Squamous cell carcinoma SCC-4 | 3.2 | Colon ca.* (SW480 met) SW620 | 3.2 |
| Testis Pool | 0.5 | Colon ca. HT29 | 0.7 |
| Prostate ca.* (bone met) PC-3 | 0.6 | Colon ca. HCT-116 | 2.6 |
| Prostate Pool | 0.2 | Colon ca. CaCo-2 | 5.3 |
| Placenta | 5.4 | Colon cancer tissue | 14.5 |
| Uterus Pool | 0.0 | Colon ca. SW1116 | 2.3 |
| Ovarian ca. OVCAR-3 | 16.3 | Colon ca. Colo-205 | 7.9 |
| Ovarian ca. SK-OV-3 | 18.7 | Colon ca. SW-48 | 2.7 |
| Ovarian ca. OVCAR-4 | 3.9 | Colon Pool | 1.8 |
| Ovarian ca. OVCAR-5 | 5.7 | Small Intestine Pool | 0.7 |
| Ovarian ca. IGROV-1 | 0.3 | Stomach Pool | 1.2 |
| Ovarian ca. OVCAR-8 | 1.3 | Bone Marrow Pool | 0.3 |
| Ovary | 3.2 | Fetal Heart | 0.5 |
| Breast ca. MCF-7 | 3.0 | Heart Pool | 1.2 |
| Breast ca. MDA-MB-231 | 4.1 | Lymph Node Pool | 2.9 |
| Breast ca. BT 549 | 100.0 | Fetal Skeletal Muscle | 0.3 |
| Breast ca. T47D | 2.0 | Skeletal Muscle Pool | 1.0 |
| Breast ca. MDA-N | 1.6 | Spleen Pool | 2.1 |
| Breast Pool | 2.0 | Thymus pool | 1.4 |
| Trachea | 2.3 | CNS cancer (glio/astro) U87-MG | 8.1 |
| Lung | 0.5 | CNS cancer (glio/astro) U-118-MG | 12.3 |
| Fetal Lung | 2.2 | CNS cancer (neuro; met) SK-N-AS | 2.0 |
| Lung ca. NCI-N417 | 0.1 | CNS cancer (astro) SF-539 | 3.4 |
| Lung ca. LX-1 | 6.1 | CNS cancer (astro) SNB-75 | 27.4 |
| Lung ca. NCI-H146 | 0.4 | CNS cancer (glio) SNB-19 | 2.4 |
| Lung ca. SHP-77 | 1.8 | CNS cancer (glio) SF-295 | 26.8 |
| Lung ca. A549 | 4.1 | Brain (Amygdala) Pool | 2.1 |
| Lung ca. NCI-H526 | 0.1 | Brain (cerebellum) | 6.9 |
| Lung ca. NCI-H23 | 3.0 | Brain (fetal) | 1.2 |
| Lung ca. NCI-H460 | 2.6 | Brain (Hippocampus) Pool | 1.9 |
| Lung ca. HOP-62 | 4.0 | Cerebral Cortex Pool | 3.8 |
| Lung ca. NCI-H522 | 1.0 | Brain (Substantia nigra) Pool | 2.6 |
| Liver | 1.4 | Brain (Thalamus) Pool | 2.8 |
| Fetal Liver | 10.4 | Brain (whole) | 5.3 |
| Liver ca. HepG2 | 8.3 | Spinal Cord Pool | 2.4 |
| Kidney Pool | 0.0 | Adrenal Gland | 3.2 |
| Fetal Kidney | 0.7 | Pituitary gland Pool | 0.6 |
| Renal ca. 786-0 | 5.3 | Salivary Gland | 2.5 |
| Renal ca. A498 | 4.0 | Thyroid (female) | 25.3 |
| Renal ca. ACHN | 3.0 | Pancreatic ca. CAPAN2 | 5.7 |
| Renal ca. UO-31 | 15.2 | Pancreas Pool | 3.0 |

TABLE ANF
HASS Panel v1.0

| | Rel. | Rel. | | Rel. | Rel. |
|---|---|---|---|---|---|
| | Exp. (%) | Exp. (%) | | Exp. (%) | Exp. (%) |
| | Ag2052, | Ag2052, | | Ag2052, | Ag2052, |
| | Run | Run | | Run | Run |
| Tissue Name | 247736616 | 248455625 | Tissue Name | 247736616 | 248455625 |
| MCF-7 C1 | 12.6 | 7.1 | U87-MG F1 (B) | 40.3 | 22.4 |
| MCF-7 C2 | 12.7 | 8.6 | U87-MG F2 | 11.1 | 6.7 |
| MCF-7 C3 | 10.2 | 5.6 | U87-MG F3 | 12.2 | 8.0 |
| MCF-7 C4 | 16.2 | 19.5 | U87-MG F4 | 27.0 | 17.8 |
| MCF-7 C5 | 13.2 | 11.0 | U87-MG F5 | 59.0 | 38.2 |
| MCF-7 C6 | 13.2 | 14.6 | U87-MG F6 | 61.1 | 44.4 |
| MCF-7 C7 | 12.7 | 10.4 | U87-MG F7 | 72.7 | 50.7 |
| MCF-7 C9 | 9.7 | 12.9 | U87-MG F8 | 75.3 | 54.7 |
| MCF-7 C10 | 15.8 | 17.1 | U87-MG F9 | 29.9 | 28.1 |
| MCF-7 C11 | 2.5 | 1.8 | U87-MG F10 | 65.1 | 50.0 |
| MCF-7 C12 | 9.9 | 8.0 | U87-MG F11 | 58.2 | 48.3 |
| MCF-7 C13 | 12.5 | 17.1 | U87-MG F12 | 47.0 | 42.6 |
| MCF-7 C15 | 5.6 | 6.5 | U87-MG F13 | 95.3 | 77.9 |
| MCF-7 C16 | 14.0 | 21.5 | U87-MG F14 | 96.6 | 80.1 |
| MCF-7 C17 | 10.2 | 6.9 | U87-MG F15 | 64.6 | 54.7 |
| T24 D1 | 25.0 | 14.4 | U87-MG F16 | 51.8 | 47.6 |
| T24 D2 | 33.0 | 42.0 | U87-MG F17 | 62.0 | 49.0 |
| T24 D3 | 29.3 | 19.1 | LnCAP A1 | 9.4 | 6.0 |
| T24 D4 | 39.8 | 30.6 | LnCAP A2 | 8.1 | 5.5 |
| T24 D5 | 28.5 | 19.5 | LnCAP A3 | 6.3 | 3.4 |
| T24 D6 | 32.8 | 27.2 | LnCAP A4 | 10.4 | 6.9 |
| T24 D7 | 18.3 | 25.9 | LnCAP A5 | 10.0 | 6.0 |
| T24 D9 | 12.1 | 8.5 | LnCAP A6 | 10.0 | 6.3 |
| T24 D10 | 23.5 | 19.2 | LnCAP A7 | 9.2 | 6.6 |
| T24 D11 | 13.2 | 11.7 | LnCAP A8 | 11.5 | 8.8 |
| T24 D12 | 24.0 | 19.2 | LnCAP A9 | 10.8 | 7.2 |
| T24 D13 | 8.5 | 5.8 | LnCAP A10 | 11.0 | 8.0 |
| T24 D15 | 10.7 | 8.0 | LnCAP A11 | 15.7 | 10.7 |
| T24 D16 | 6.6 | 4.7 | LnCAP A12 | 3.5 | 2.3 |
| T24 D17 | 12.0 | 7.4 | LnCAP A13 | 5.7 | 3.3 |
| CAPaN B1 | 64.6 | 52.1 | LnCAP A14 | 3.3 | 1.7 |
| CAPaN B2 | 46.3 | 33.2 | LnCAP A15 | 2.5 | 1.3 |
| CAPaN B3 | 13.0 | 10.7 | LnCAP A16 | 12.5 | 8.6 |
| CAPaN B4 | 39.8 | 30.4 | LnCAP A17 | 12.2 | 2.5 |
| CAPaN B5 | 39.5 | 28.7 | Primary Astrocytes | 47.3 | 27.9 |
| CAPaN B6 | 27.5 | 25.7 | Primary Renal | 100.0 | 100.0 |
| | | | Proximal Tubule | | |
| | | | Epithelial cell A2 | | |
| CAPaN B7 | 30.1 | 31.2 | Primary melanocytes | 40.1 | 21.8 |
| | | | A5 | | |
| CAPaN B8 | 33.2 | 26.8 | 126443-341 medullo | 0.7 | 0.4 |
| CAPaN B9 | 38.7 | 50.0 | 126444-487 medullo | 2.2 | 1.8 |
| CAPaN B10 | 57.4 | 51.4 | 126445-425 medullo | 1.6 | 1.0 |
| CAPaN B11 | 45.1 | 28.5 | 126446-690 medullo | 4.4 | 2.6 |
| CAPaN B12 | 31.4 | 22.7 | 126447-54 adult | 33.4 | 22.2 |
| | | | glioma | | |
| CAPaN B13 | 38.7 | 29.7 | 126448-245 adult | 9.4 | 6.3 |
| | | | glioma | | |
| CAPaN B14 | 29.9 | 22.1 | 126449-317 adult | 10.4 | 6.0 |
| | | | glioma | | |
| CAPaN B15 | 32.8 | 20.7 | 126450-212 glioma | 41.5 | 22.8 |
| CAPaN B16 | 29.7 | 16.4 | 126451-456 glioma | 17.4 | 11.3 |
| CAPaN B17 | 42.3 | 24.3 | | | |

TABLE ANG
Panel 1.3D

| | Rel. | | Rel. |
|---|---|---|---|
| | Exp. (% | | Exp. (%) |
| | Ag2052, | | Ag2052, |
| | Run | | Run |
| Tissue Name | 166004256 | Tissue Name | 166004256 |
| Liver adenocarcinoma | 21.8 | Kidney (fetal) | 19.2 |
| Pancreas | 4.2 | Renal ca. 786-0 | 8.4 |
| Pancreatic ca. CAPAN 2 | 24.5 | Renal ca. A498 | 26.4 |
| Adrenal gland | 11.7 | Renal ca. RXF 393 | 34.4 |
| Thyroid | 37.6 | Renal ca. ACHN | 9.3 |
| Salivary gland | 25.3 | Renal ca. UO-31 | 33.7 |
| Pituitary gland | 13.8 | Renal ca. TK-10 | 2.8 |
| Brain (fetal) | 11.7 | Liver | 14.0 |
| Brain (whole) | 51.4 | Liver (fetal) | 16.2 |
| Brain (amygdala) | 29.5 | Liver ca. (hepatoblast) HepG2 | 33.9 |
| Brain (cerebellum) | 24.3 | Lung | 22.8 |
| Brain (hippocampus) | 24.5 | Lung (fetal) | 10.7 |
| Brain (substantia nigra) | 17.8 | Lung ca. (small cell) LX-1 | 25.2 |
| Brain (thalamus) | 27.5 | Lung ca. (small cell) NCI-H69 | 2.1 |
| Cerebral Cortex | 45.4 | Lung ca. (s.cell var.) SHP-77 | 6.9 |
| Spinal cord | 30.4 | Lung ca. (large cell)NCI-H460 | 2.1 |
| glio/astro U87-MG | 42.6 | Lung ca. (non-sm. cell) A549 | 4.4 |
| glio/astro U-118-MG | 23.5 | Lung ca. (non-s.cell) NCI-H23 | 4.4 |
| astrocytoma SW1783 | 24.3 | Lung ca. (non-s.cell) HOP-62 | 30.4 |
| neuro*; met SK-N-AS | 5.4 | Lung ca. (non-s.cl) NCI-H522 | 3.4 |
| astrocytoma SF-539 | 43.8 | Lung ca. (squam.) SW 900 | 18.4 |
| astrocytoma SNB-75 | 21.9 | Lung ca. (squam.) NCI-H596 | 1.9 |
| glioma SNB-19 | 20.7 | Mammary gland | 15.5 |
| glioma U251 | 43.2 | Breast ca.* (pl.ef) MCF-7 | 10.7 |
| glioma SF-295 | 25.5 | Breast ca.* (pl.ef) MDA-MB-231 | 13.2 |
| Heart (fetal) | 15.2 | Breast ca.* (pl.ef) T47D | 6.0 |
| Heart | 13.7 | Breast ca. BT-549 | 100.0 |
| Skeletal muscle (fetal) | 8.2 | Breast ca. MDA-N | 3.7 |
| Skeletal muscle | 11.8 | Ovary | 23.5 |
| Bone marrow | 19.5 | Ovarian ca. OVCAR-3 | 14.1 |
| Thymus | 7.7 | Ovarian ca. OVCAR-4 | 20.7 |
| Spleen | 34.6 | Ovarian ca. OVCAR-5 | 23.5 |
| Lymph node | 17.4 | Ovarian ca. OVCAR-8 | 7.8 |
| Colorectal | 12.5 | Ovarian ca. IGROV-1 | 5.1 |
| Stomach | 8.0 | Ovarian ca.* (ascites) SK-OV-3 | 27.9 |
| Small intestine | 12.2 | Uterus | 11.0 |
| Colon ca. SW480 | 9.7 | Placenta | 40.3 |
| Colon ca.* SW620(SW480 met) | 5.9 | Prostate | 8.0 |
| Colon ca. HT29 | 1.2 | Prostate ca.* (bone met)PC-3 | 8.4 |
| Colon ca. HCT-116 | 4.8 | Testis | 4.3 |
| Colon ca. CaCo-2 | 15.7 | Melanoma Hs688(A).T | 22.7 |
| Colon ca. tissue(ODO3866) | 62.4 | Melanoma* (met) Hs688(B).T | 21.8 |
| Colon ca. HCC-2998 | 12.9 | Melanoma UACC-62 | 23.0 |
| Gastric ca.* (liver met) NCI-N87 | 21.9 | Melanoma M14 | 43.2 |
| Bladder | 11.4 | Melanoma LOX IMVI | 11.2 |
| Trachea | 13.1 | Melanoma* (met) SK-MEL-5 | 22.8 |
| Kidney | 31.0 | Adipose | 12.8 |

TABLE ANH
Panel 2.2

| | Rel. | | Rel. |
|---|---|---|---|
| | Exp. %) | | Exp. (%) |
| | Ag2052, | | Ag2052, |
| | Run | | Run |
| Tissue Name | 174244470 | Tissue Name | 174244470 |
| Normal Colon | 3.3 | Kidney Margin (OD04348) | 13.1 |
| Colon cancer (OD06064) | 23.3 | Kidney malignant cancer | 1.0 |
| | | (OD06204B) | |
| Colon Margin (OD06064) | 3.6 | Kidney normal adjacent tissue | 9.5 |
| | | (OD06204E) | |
| Colon cancer (OD06159) | 1.5 | Kidney Cancer (OD04450-01) | 22.2 |
| Colon Margin (OD06159) | 3.6 | Kidney Margin (OD04450-03) | 4.5 |
| Colon cancer (OD06297-04) | 1.3 | Kidney Cancer 8120613 | 0.6 |
| Colon Margin (OD06297-05) | 4.7 | Kidney Margin 8120614 | 0.0 |
| CC Gr.2 ascend colon (ODO3921) | 1.5 | Kidney Cancer 9010320 | 10.7 |
| CC Margin (ODO3921) | 2.6 | Kidney Margin 9010321 | 6.6 |
| Colon cancer metastasis | 6.7 | Kidney Cancer 8120607 | 9.7 |
| (OD06104) | | | |
| Lung Margin (OD06104) | 6.0 | Kidney Margin 8120608 | 11.4 |
| Colon mets to lung (OD04451-01) | 12.8 | Normal Uterus | 3.1 |
| Lung Margin (OD04451-02) | 6.0 | Uterine Cancer 064011 | 3.5 |
| Normal Prostate | 2.3 | Normal Thyroid | 7.2 |
| Prostate Cancer (OD04410) | 0.7 | Thyroid Cancer 064010 | 44.8 |
| Prostate Margin (OD04410) | 1.2 | Thyroid Cancer A302152 | 100.0 |
| Normal Ovary | 6.1 | Thyroid Margin A302153 | 7.6 |
| Ovarian cancer (OD06283-03) | 4.1 | Normal Breast | 2.2 |
| Ovarian Margin (OD06283-07) | 2.0 | Breast Cancer (OD04566) | 2.5 |
| Ovarian Cancer 064008 | 9.2 | Breast Cancer 1024 | 6.3 |
| Ovarian cancer (OD06145) | 8.9 | Breast Cancer (OD04590-01) | 8.5 |
| Ovarian Margin (OD06145) | 3.8 | Breast Cancer Mets | 4.4 |
| | | (OD04590-03) | |
| Ovarian cancer (OD06455-03) | 6.1 | Breast Cancer Metastasis | 3.3 |
| | | (OD04655-05) | |
| Ovarian Margin (OD06455-07) | 1.0 | Breast Cancer 064006 | 4.9 |
| Normal Lung | 4.9 | Breast Cancer 9100266 | 2.7 |
| Invasive poor diff. lung adeno | 2.9 | Breast Margin 9100265 | 1.7 |
| (ODO4945-01 | | | |
| Lung Margin (ODO4945-03) | 3.2 | Breast Cancer A209073 | 1.5 |
| Lung Malignant Cancer | 11.1 | Breast Margin A2090734 | 2.3 |
| (OD03126) | | | |
| Lung Margin (OD03126) | 5.1 | Breast cancer (OD06083) | 4.4 |
| Lung Cancer (OD05014A) | 19.6 | Breast cancer node metastasis | 5.6 |
| | | (OD06083) | |
| Lung Margin (OD05014B) | 15.3 | Normal Liver | 6.9 |
| Lung cancer (OD06081) | 3.4 | Liver Cancer 1026 | 8.0 |
| Lung Margin (OD06081) | 1.3 | Liver Cancer 1025 | 22.2 |
| Lung Cancer (OD04237-01) | 4.6 | Liver Cancer 6004-T | 13.8 |
| Lung Margin (OD04237-02) | 11.1 | Liver Tissue 6004-N | 4.1 |
| Ocular Melanoma Metastasis | 3.5 | Liver Cancer 6005-T | 21.5 |
| Ocular Melanoma Margin (Liver) | 9.8 | Liver Tissue 6005-N | 51.1 |
| Melanoma Metastasis | 5.4 | Liver Cancer 064003 | 13.6 |
| Melanoma Margin (Lung) | 5.1 | Normal Bladder | 2.8 |
| Normal Kidney | 3.3 | Bladder Cancer 1023 | 4.8 |
| Kidney Ca, Nuclear grade 2 | 5.0 | Bladder Cancer A302173 | 6.1 |
| (OD04338) | | | |
| Kidney Margin (OD04338) | 10.6 | Normal Stomach | 5.3 |
| Kidney Ca Nuclear grade 1/2 | 15.0 | Gastric Cancer 9060397 | 6.3 |
| (OD04339) | | | |
| Kidney Margin (OD04339) | 11.3 | Stomach Margin 9060396 | 5.0 |
| Kidney Ca, Clear cell type | 4.2 | Gastric Cancer 9060395 | 4.6 |
| (OD04340) | | | |
| Kidney Margin (OD04340) | 7.2 | Stomach Margin 9060394 | 7.7 |
| Kidney Ca, Nuclear grade 3 | 3.1 | Gastric Cancer 064005 | 3.8 |
| (OD04348) | | | |

TABLE ANI
Panel 4.1D

| | Rel. | | Rel. |
|---|---|---|---|
| | Exp. (% | | Exp. (%) |
| | Ag5278, | | Ag5278, |
| | Run | | Run |
| Tissue Name | 230472911 | Tissue Name | 230472911 |
| Secondary Th1 act | 3.4 | HUVEC IL-1beta | 13.0 |
| Secondary Th2 act | 3.3 | HUVEC IFN gamma | 9.0 |
| Secondary Tr1 act | 1.2 | HUVEC TNF alpha + IFN gamma | 7.4 |
| Secondary Th1 rest | 0.4 | HUVEC TNF alpha + IL4 | 2.1 |
| Secondary Th2 rest | 0.0 | HUVEC IL-11 | 3.6 |
| Secondary Tr1 rest | 0.0 | Lung Microvascular EC none | 27.7 |
| Primary Th1 act | 0.0 | Lung Microvascular EC TNF alpha + IL-1beta | 8.2 |
| Primary Th2 act | 1.1 | Microvascular Dermal EC none | 4.2 |
| Primary Tr1 act | 1.4 | Microsvasular Dermal EC | 3.0 |
| | | TNF alpha + IL-1beta | |
| Primary Th1 rest | 0.5 | Bronchial epithelium TNF alpha + IL1beta | 9.1 |
| Primary Th2 rest | 0.5 | Small airway epithelium none | 22.1 |
| Primary Tr1 rest | 0.9 | Small airway epithelium TNF alpha + IL-1beta | 33.9 |
| CD45RA CD4 lymphocyte act | 5.0 | Coronery artery SMC rest | 6.2 |
| CD45RO CD4 lymphocyte act | 1.6 | Coronery artery SMC TNF alpha + IL-1beta | 11.3 |
| CD8 lymphocyte act | 0.4 | Astrocytes rest | 2.3 |
| Secondary CD8 lymphocyte rest | 1.3 | Astrocytes TNF alpha + IL-1beta | 3.1 |
| Secondary CD8 lymphocyte act | 0.0 | KU-812 (Basophil) rest | 1.9 |
| CD4 lymphocyte none | 0.0 | KU-812 (Basophil) | 10.9 |
| | | PMA/ionomycin | |
| 2ry Th1/Th2/Tr1_anti-CD95 | 0.0 | CCD1106 (Keratinocytes) none | 5.8 |
| CH11 | | | |
| LAK cells rest | 18.6 | CCD1106 (Keratinocytes) | 4.8 |
| | | TNF alpha + IL-1beta | |
| LAK cells IL-2 | 0.6 | Liver cirrhosis | 1.9 |
| LAK cells IL-2 + IL-12 | 0.0 | NCI-H292 none | 7.1 |
| LAK cells IL-2 + IFN gamma | 0.7 | NCI-H292 IL-4 | 8.4 |
| LAK cells IL-2 + IL-18 | 0.9 | NCI-H292 IL-9 | 7.0 |
| LAK cells PMA/ionomycin | 62.4 | NCI-H292 IL-13 | 5.6 |
| NK Cells IL-2 rest | 1.0 | NCI-H292 IFN gamma | 3.6 |
| Two Way MLR 3 day | 9.4 | HPAEC none | 9.1 |
| Two Way MLR 5 day | 3.9 | HPAEC TNF alpha + IL-1beta | 28.3 |
| Two Way MLR 7 day | 2.3 | Lung fibroblast none | 9.3 |
| PBMC rest | 0.6 | Lung fibroblast TNF alpha + IL- | 12.2 |
| | | 1beta | |
| PBMC PWM | 1.1 | Lung fibroblast IL-4 | 3.9 |
| PBMC PHA-L | 2.2 | Lung fibroblast IL-9 | 11.8 |
| Ramos (B cell) none | 0.0 | Lung fibroblast IL-13 | 5.4 |
| Ramos (B cell) ionomycin | 0.0 | Lung fibroblast IFN gamma | 19.5 |
| B lymphocytes PWM | 0.0 | Dermal fibroblast CCD1070 rest | 32.1 |
| B lymphocytes CD40L and IL-4 | 1.4 | Dermal fibroblast CCD1070 TNF | 66.0 |
| | | alpha | |
| EOL-1 dbcAMP | 1.4 | Dermal fibroblast CCD1070 IL- | 21.8 |
| | | 1beta | |
| EOL-1 dbcAMP | 1.4 | Dermal fibroblast IFN gamma | 42.3 |
| PMA/ionomycin | | | |
| Dendritic cells none | 100.0 | Dermal fibroblast IL-4 | 45.1 |
| Dendritic cells LPS | 34.9 | Dermal Fibroblasts rest | 15.7 |
| Dendritic cells anti-CD40 | 44.8 | Neutrophils TNFa + LPS | 0.0 |
| Monocytes rest | 1.4 | Neutrophils rest | 0.6 |
| Monocytes LPS | 19.9 | Colon | 0.0 |
| Macrophages rest | 12.5 | Lung | 1.4 |
| Macrophages LPS | 11.2 | Thymus | 0.0 |
| HUVEC none | 5.9 | Kidney | 12.8 |
| HUVEC starved | 11.7 | | |

TABLE ANJ
Panel 4D

| | Rel. | | Rel. |
|---|---|---|---|
| | Exp. ( ) | | Exp. (%) |
| | Ag2052, | | Ag2052, |
| | Run | | Run |
| Tissue Name | 161706487 | Tissue Name | 161706487 |
| Secondary Th1 act | 2.6 | HUVEC IL-1beta | 2.1 |
| Secondary Th2 act | 1.7 | HUVEC IFN gamma | 5.2 |
| Secondary Tr1 act | 1.9 | HUVEC TNF alpha + IFN gamma | 5.7 |
| Secondary Th1 rest | 0.3 | HUVEC TNF alpha + IL4 | 4.5 |
| Secondary Th2 rest | 0.5 | HUVEC IL-11 | 2.6 |
| Secondary Tr1 rest | 0.6 | Lung Microvascular EC none | 9.9 |
| Primary Th1 act | 1.4 | Lung Microvascular EC TNF alpha + IL-1beta | 10.0 |
| Primary Th2 act | 0.7 | Microvascular Dermal EC none | 16.6 |
| Primary Tr1 act | 1.2 | Microsvasular Dermal EC | 9.2 |
| | | TNF alpha + IL-1beta | |
| Primary Th1 rest | 2.2 | Bronchial epithelium TNF alpha + IL1beta | 3.1 |
| Primary Th2 rest | 1.4 | Small airway epithelium none | 12.5 |
| Primary Tr1 rest | 0.2 | Small airway epithelium TNF alpha + IL-1beta | 46.0 |
| CD45RA CD4 lymphocyte act | 4.2 | Coronery artery SMC rest | 5.4 |
| CD45RO CD4 lymphocyte act | 1.4 | Coronery artery SMC TNF alpha + IL-1beta | 4.3 |
| CD8 lymphocyte act | 0.3 | Astrocytes rest | 2.2 |
| Secondary CD8 lymphocyte rest | 1.4 | Astrocytes TNF alpha + IL-1beta | 2.0 |
| Secondary CD8 lymphocyte act | 0.4 | KU-812 (Basophil) rest | 1.5 |
| CD4 lymphocyte none | 0.4 | KU-812 (Basophil) | 11.0 |
| | | PMA/ionomycin | |
| 2ry Th1/Th2/Tr1_anti-CD95 | 0.8 | CCD1106 (Keratinocytes) none | 3.1 |
| CH11 | | | |
| LAK cells rest | 43.2 | CCD1106 (Keratinocytes) | 0.8 |
| | | TNF alpha + IL-1beta | |
| LAK cells IL-2 | 0.8 | Liver cirrhosis | 1.5 |
| LAK cells IL-2 + IL-12 | 1.8 | Lupus kidney | 0.7 |
| LAK cells IL-2 + IFN gamma | 3.2 | NCI-H292 none | 5.8 |
| LAK cells IL-2 + IL-18 | 2.1 | NCI-H292 IL-4 | 5.5 |
| LAK cells PMA/ionomycin | 26.2 | NCI-H292 IL-9 | 7.4 |
| NK Cells IL-2 rest | 0.3 | NCI-H292 IL-13 | 2.7 |
| Two Way MLR 3 day | 9.2 | NCI-H292 IFN gamma | 3.3 |
| Two Way MLR 5 day | 9.3 | HPAEC none | 5.6 |
| Two Way MLR 7 day | 2.0 | HPAEC TNF alpha + IL-1beta | 10.7 |
| PBMC rest | 1.0 | Lung fibroblast none | 6.3 |
| PBMC PWM | 5.3 | Lung fibroblast TNF alpha + IL- | 6.3 |
| | | 1beta | |
| PBMC PHA-L | 5.0 | Lung fibroblast IL-4 | 10.4 |
| Ramos (B cell) none | 0.0 | Lung fibroblast IL-9 | 8.1 |
| Ramos (B cell) ionomycin | 0.0 | Lung fibroblast IL-13 | 5.6 |
| B lymphocytes PWM | 2.2 | Lung fibroblast IFN gamma | 15.4 |
| B lymphocytes CD40L and IL-4 | 1.2 | Dermal fibroblast CCD1070 rest | 15.5 |
| EOL-1 dbcAMP | 0.7 | Dermal fibroblast CCD1070 TNF | 18.9 |
| | | alpha | |
| EOL-1 dbcAMP | 1.5 | Dermal fibroblast CCD1070 IL- | 11.1 |
| PMA/ionomycin | | 1beta | |
| Dendritic cells none | 66.9 | Dermal fibroblast IFN gamma | 19.6 |
| Dendritic cells LPS | 37.6 | Dermal fibroblast IL-4 | 21.2 |
| Dendritic cells anti-CD40 | 77.9 | IBD Colitis 2 | 0.2 |
| Monocytes rest | 5.1 | IBD Crohn's | 0.5 |
| Monocytes LPS | 17.2 | Colon | 3.9 |
| Macrophages rest | 100.0 | Lung | 19.8 |
| Macrophages LPS | 40.9 | Thymus | 12.9 |
| HUVEC none | 5.3 | Kidney | 2.4 |
| HUVEC starved | 10.6 | | |

TABLE ANK
Panel 5 Islet

| | Rel. | | Rel. |
|---|---|---|---|
| | Exp. (%) | | Exp. (%) |
| | Ag2052, | | Ag2052, |
| | un | | Run |
| Tissue Name | 279370795 | Tissue Name | 279370795 |
| 97457_Patient-02go_adipose | 15.6 | 94709_Donor 2 AM - A_adipose | 24.7 |
| 97476_Patient-07sk_skeletal | 0.0 | 94710_Donor 2 AM - B_adipose | 24.7 |
| muscle | | | |
| 97477_Patient-07ut_uterus | 22.1 | 94711_Donor 2 AM - C_adipose | 14.7 |
| 97478_Patient-07pl_placenta | 13.1 | 94712_Donor 2 AD - A_adipose | 64.2 |
| 99167_Bayer Patient 1 | 17.6 | 94713_Donor 2 AD - B_adipose | 89.5 |
| 97482_Patient-08ut_uterus | 15.3 | 94714_Donor 2 AD - C_adipose | 66.4 |
| 97483_Patient-08pl_placenta | 11.6 | 94742_Donor 3 U - A_Mesenchymal | 17.3 |
| | | Stem Cells | |
| 97486_Patient-09sk_skeletal | 4.8 | 94743_Donor 3 U - B_Mesenchymal | 23.2 |
| muscle | | Stem Cells | |
| 97487_Patient-09ut_uterus | 15.5 | 94730_Donor 3 AM - A_adipose | 54.0 |
| 97488_Patient-09pl_placenta | 7.9 | 94731_Donor 3 AM - B_adipose | 76.3 |
| 97492_Patient-10ut_uterus | 14.5 | 94732_Donor 3 AM - C_adipose | 59.9 |
| 97493_Patient-10pl_placenta | 23.8 | 94733_Donor 3 AD - A_adipose | 100.0 |
| 97495_Patient-11go_adipose | 11.9 | 94734_Donor 3 AD - B_adipose | 92.0 |
| 97496_Patient-11sk_skeletal | 3.2 | 94735_Donor 3 AD - C_adipose | 32.1 |
| muscle | | | |
| 97497_Patient-11ut_uterus | 36.9 | 77138_Liver_HepG2untreated | 62.9 |
| 97498_Patient-11pl_placenta | 7.0 | 73556_Heart_Cardiac stromal cells | 0.3 |
| | | (primary) | |
| 97500_Patient-12go_adipose | 17.2 | 81735_Small Intestine | 10.9 |
| 97501_Patient-12sk_skeletal | 8.4 | 72409_Kidney_Proximal Convoluted | 23.7 |
| muscle | | Tubule | |
| 97502_Patient-12ut_uterus | 25.2 | 82685_Small intestine_Duodenum | 9.3 |
| 97503_Patient-12pl_placenta | 23.8 | 90650_Adrenal_Adrenocortical | 8.4 |
| | | adenoma | |
| 94721_Donor 2 U - | 61.6 | 72410_Kidney_HRCE | 40.1 |
| A_Mesenchymal Stem Cells | | | |
| 94722_Donor 2 U - | 45.1 | 72411_Kidney_HRE | 13.5 |
| B_Mesenchymal Stem Cells | | | |
| 94723_Donor 2 U - | 53.2 | 73139_Uterus_Uterine smooth | 61.1 |
| C_Mesenchymal Stem Cells | | muscle cells | |

AI_comprehensive panel_v1.0 Summary: Ag2052 Highest expression of this gene is detected in synovium from an orthoarthritis (OA) patient (CT=20.3). High levels of expression of this gene are detected in samples derived from normal and orthoarthitis/rheumatoid arthritis bone and adjacent bone, cartilage, synovium and synovial fluid samples, from normal lung, COPD lung, emphysema, atopic asthma, asthma, allergy, Crohn's disease (normal matched control and diseased), ulcerative colitis(normal matched control and diseased), and psoriasis (normal matched control and diseased). Therefore, therapeutic modulation of this gene product may ameliorate symptoms/conditions associated with autoimmune and inflammatory disorders including psoriasis, allergy, asthma, inflammatory bowel disease, rheumatoid arthritis and osteoarthritis.

CNS_neurodegeneration_v1.0 Summary: Ag5277/Ag5278 Expression of this gene is low/undetectable (CTs>35) across all of the samples on this panel.

General_screen_panel_v1.5 Summary: Ag5278 Highest expression of this gene is detected in breast cancer BT-549 cell line (CT=29). Moderate levels of expression of this gene is also seen in cluster of cancer cell lines derived from pancreatic, gastric, colon, lung, liver, renal, breast, ovarian, melanoma and brain cancers. In addition, moderate to low levels of expression of this gene is also seen in all the regions of brain, in tissues with metabolic/endocrine functions such as pancreas, adrenal gland, thyroid, fetal liver and colon. Please see panel 1.3D for further discussion of this gene.

Ag5277 Expression of this gene is low/undetectable (CTs>35) across all of the samples on this panel.

HASS Panel v1.0 Summary: Ag2052 Two experiments with same probe and primer sets are in excellent agreement. This gene shows wide spread expression in this panel, with highest expression in primary renal proximal tubular epithelial cells cultured in vitro (CTs=20-22). The expression of this gene is also higher in the glioblastoma type of brain cancer compared to the medulloblastoma suggesting that it may play a role in glioblastoma development than medulloblastomas. Expression is also induced in the U87-MG(cells when they are deprived of nutrients, oxygen and exposed to an acidic pH than in the control population (comparing the control U87-MG F4 with U87-MG F5, F7, F10). This suggests that the serum-starved, hypoxic and acidotic regions of brain cancers may express this gene at a higher level and that this may be used as a marker for these regions.

Panel 1.3D Summary: Ag2052 This gene shows a widespread expression in this panel. Highest expression of this gene is detected in breast cancer BT-549 cell line (CT=24.9). High levels of expression of this gene is also seen in cluster of cancer cell lines derived from pancreatic, gastric, colon, lung, liver, renal, breast, ovarian, prostate, melanoma and brain cancers. Thus, expression of this gene could be used as a marker to detect the presence of these cancers. Furthermore, therapeutic modulation of the expression or function of this gene may be effective in the treatment of pancreatic, gastric, colon, lung, liver, renal, breast, ovarian, prostate, melanoma and brain cancers.

Among tissues with metabolic or endocrine function, this gene is expressed at high levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

In addition, this gene is expressed at high levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

Panel 2.2 Summary: Ag2052 Highest expression of this gene is detected in thyroid cancer (CT=23.9). High to moderate levels of expression of this gene is also seen in normal and cancer samples derived from melanoma, colon, gastric, bladder, liver, breast, thyroid, uterine, kidney, lung, ovarian and prostate cancers. Interestingly, higher levels of expression of this gene is associated with kidney and thyroid cancers as compared to corresponding normal tissue. Therefore, expression of this gene may bay used as diagnostic marker to detect the presence of these cancers. Furthermore, therapeutic modulation of this gene may be useful in the treatment of melanoma, colon, gastric, bladder, liver, breast, thyroid, uterine, kidney, lung, ovarian and prostate cancers.

Panel 4.1D Summary: Ag5278 Highest levels of expression of this gene is detected in resting dendritic cells (CT=32). Moderate to low levels of expression of this gene is also seen in activated dendrict cells, PMA/ionomycin stimulated LAK cells, LPS activated macrophage, lung rmicrovascular endothelial cells, activated HPAEC cells, small airway epithelium, and dermal fibroblasts. Therefore, therapeutic modulation of this gene or its protein product may alter the functions associated with these cell types and would be beneficial in the treatment of autoimmune and inflammatory diseases such as asthma, allergies, inflammatory bowel disease, lupus erythematosus, psoriasis, rheumatoid arthritis, and osteoarthritis.

Ag5277 Expression of this gene is low/undetectable (CTs>35) across all of the samples on this panel.

Panel 4D Summary: Ag2052 Highest expression of this gene is detected in resting macrophage (CT=21). This gene is expressed at high to moderate levels in a wide range of cell types of significance in the immune response in health and disease. These cells include members of the T-cell, B-cell, dendritic cells, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screen_panel_v1.3 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Panel 5 Islet Summary: Ag2052 Highest expression of this gene is detected in a differentiated adipose tissue (CT=24.4). Moderate to high levels of expression is seen in placenta, uterus, adipose, skeletal muscle, small intestine, heart and kidney. This gene shows a ubiquitous expression which correlates to the expression in panel 1.3D. Please see panel 1.3D for further discussion of this gene.

AO. CG56836-04: Cathepsin B.

Expression of gene CG56836-04 was assessed using the primer-probe set Ag5264, described in Table AOA. Results of the RTQ-PCR runs are shown in Tables AOB, AOC and AOD.

TABLE AOA

Probe Name Ag5264

| | | | Start | SEQ ID |
|---|---|---|---|---|
| Primers | | Length | Position | No |
| Forward | 5′-tcctgctgggtttctggt-3′ | 18 | 455 | 411 |
| Probe | TET-5′-ccgtactccatccctccctgtgagc-3′-TAMRA | 25 | 503 | 412 |
| Reverse | 5′-tgtttgtaggtogggctgta-3′ | 20 | 605 | 413 |

TABLE AOB
CNS_neurodegeneration_v1.0

| | Rel. | | Rel. |
|---|---|---|---|
| | Exp. (%) | | Exp. (%) |
| | Ag5264, | | Ag5264, |
| | Run | | Run |
| Tissue Name | 230512807 | issue Name | 230512807 |
| AD 1 Hippo | 10.2 | Control (Path) 3 Temporal Ctx | 3.6 |
| AD 2 Hippo | 32.5 | Control (Path) 4 Temporal Ctx | 18.4 |
| AD 3 Hippo | 9.3 | AD 1 Occipital Ctx | 14.7 |
| AD 4 Hippo | 3.8 | AD 2 Occipital Ctx (Missing) | 0.0 |
| AD 5 hippo | 94.0 | AD 3 Occipital Ctx | 7.3 |
| AD 6 Hippo | 66.9 | AD 4 Occipital Ctx | 13.4 |
| Control 2 Hippo | 25.0 | AD 5 Occipital Ctx | 15.3 |
| Control 4 Hippo | 13.0 | AD 6 Occipital Ctx | 39.0 |
| Control (Path) 3 Hippo | 4.0 | Control 1 Occipital Ctx | 5.9 |
| AD 1 Temporal Ctx | 9.8 | Control 2 Occipital Ctx | 53.6 |
| AD 2 Temporal Ctx | 25.2 | Control 3 Occipital Ctx | 8.4 |
| AD 3 Temporal Ctx | 3.9 | Control 4 Occipital Ctx | 6.3 |
| AD 4 Temporal Ctx | 7.5 | Control (Path) 1 Occipital Ctx | 83.5 |
| AD 5 Inf Temporal Ctx | 74.7 | Control (Path) 2 Occipital Ctx | 6.0 |
| AD 5 SupTemporal Ctx | 43.8 | Control (Path) 3 Occipital Ctx | 1.7 |
| AD 6 Inf Temporal Ctx | 71.2 | Control (Path) 4 Occipital Ctx | 13.1 |
| AD 6 Sup Temporal Ctx | 41.8 | Control 1 Parietal Ctx | 2.9 |
| Control 1 Temporal Ctx | 5.9 | Control 2 Parietal Ctx | 30.1 |
| Control 2 Temporal Ctx | 45.1 | Control 3 Parietal Ctx | 12.3 |
| Control 3 Temporal Ctx | 12.0 | Control (Path) 1 Parietal Ctx | 100.0 |
| Control 4 Temporal Ctx | 6.7 | Control (Path) 2 Parietal Ctx | 12.6 |
| Control (Path) 1 Temporal Ctx | 47.3 | Control (Path) 3 Parietal Ctx | 2.5 |
| Control (Path) 2 Temporal Ctx | 15.9 | Control (Path) 4 Parietal Ctx | 44.1 |

TABLE AOC
General_screening_panel_v1.5

| | Rel. | | Rel. |
|---|---|---|---|
| | Exp. (%) | | Exp. (%) |
| | Ag5264, | | Ag5264, |
| | Run | | Run |
| Tissue Name | 232936651 | issue Name | 232936651 |
| Adipose | 0.7 | Renal ca. TK-10 | 3.6 |
| Melanoma* Hs688(A).T | 19.5 | Bladder | 3.8 |
| Melanoma* Hs688(B).T | 9.0 | Gastric ca. (liver met.) NCI-N87 | 10.2 |
| Melanoma* M14 | 24.7 | Gastric ca. KATO III | 5.5 |
| Melanoma* LOXIMVI | 15.6 | Colon ca. SW-948 | 1.2 |
| Melanoma* SK-MEL-5 | 9.7 | Colon ca. SW480 | 7.0 |
| Squamous cell carcinoma SCC-4 | 3.1 | Colon ca.* (SW480 met) SW620 | 2.0 |
| Testis Pool | 0.4 | Colon ca. HT29 | 0.6 |
| Prostate ca.* (bone met) PC-3 | 2.0 | Colon ca. HCT-116 | 3.1 |
| Prostate Pool | 0.6 | Colon ca. CaCo-2 | 5.2 |
| Placenta | 3.7 | Colon cancer tissue | 8.6 |
| Uterus Pool | 0.2 | Colon ca. SW1116 | 2.4 |
| Ovarian ca. OVCAR-3 | 6.7 | Colon ca. Colo-205 | 4.1 |
| Ovarian ca. SK-OV-3 | 7.2 | Colon ca. SW-48 | 1.3 |
| Ovarian ca. OVCAR-4 | 4.2 | Colon Pool | 1.2 |
| Ovarian ca. OVCAR-5 | 6.2 | Small Intestine Pool | 0.7 |
| Ovarian ca. IGROV-1 | 1.5 | Stomach Pool | 1.3 |
| Ovarian ca. OVCAR-8 | 2.2 | Bone Marrow Pool | 0.7 |
| Ovary | 1.4 | Fetal Heart | 0.5 |
| Breast ca. MCF-7 | 2.7 | Heart Pool | 1.3 |
| Breast ca. MDA-MB-231 | 4.9 | Lymph Node Pool | 2.2 |
| Breast ca. BT 549 | 100.0 | Fetal Skeletal Muscle | 0.3 |
| Breast ca. T47D | 1.3 | Skeletal Muscle Pool | 1.3 |
| Breast ca. MDA-N | 1.1 | Spleen Pool | 1.2 |
| Breast Pool | 1.7 | Thymus Pool | 0.9 |
| Trachea | 3.0 | CNS cancer (glio/astro) U87-MG | 12.6 |
| Lung | 0.2 | CNS cancer (glio/astro) U-118-MG | 9.0 |
| Fetal Lung | 1.6 | CNS cancer (neuro; met) SK-N-AS | 2.1 |
| Lung ca. NCI-N417 | 0.2 | CNS cancer (astro) SF-539 | 7.4 |
| Lung ca. LX-1 | 4.5 | CNS cancer (astro) SNB-75 | 22.5 |
| Lung ca. NCI-H146 | 0.2 | CNS cancer (glio) SNB-19 | 1.7 |
| Lung ca. SHP-77 | 1.6 | CNS cancer (glio) SF-295 | 15.6 |
| Lung ca. A549 | 4.1 | Brain (Amygdala) Pool | 1.4 |
| Lung ca. NCI-H526 | 0.2 | Brain (cerebellum) | 5.6 |
| Lung ca. NCI-H23 | 2.2 | Brain (fetal) | 1.0 |
| Lung ca. NCI-H460 | 1.2 | Brain (Hippocampus) Pool | 1.3 |
| Lung ca. HOP-62 | 5.6 | Cerebral Cortex Pool | 1.6 |
| Lung ca. NCI-H522 | 1.4 | Brain (Substantia nigra) Pool | 1.5 |
| Liver | 1.7 | Brain (Thalamus) Pool | 2.1 |
| Fetal Liver | 4.9 | Brain (whole) | 3.1 |
| Liver ca. HepG2 | 4..9 | Spinal Cord Pool | 1.6 |
| Kidney Pool | 2.4 | Adrenal Gland | 2.1 |
| Fetal Kidney | 1.0 | Pituitary gland Pool | 0.4 |
| Renal ca. 786-0 | 1.0 | Salivary Gland | 1.6 |
| Renal ca. A498 | 1.7 | Thyroid (female) | 16.7 |
| Renal ca. ACHN | 4.0 | Pancreatic ca. CAPAN2 | 5.6 |
| Renal ca. UO-31 | 11.2 | Pancreas Pool | 2.8 |

TABLE AOD
Panel 4.1D

| | Rel. | | Rel. |
|---|---|---|---|
| | Exp. (% | | Exp. (%) |
| | Ag5264, | | Ag5264, |
| | Run | | Run |
| Tissue Name | 230472870 | Tissue Name | 230472870 |
| Secondary Th1 act | 4.0 | HUVEC IL-1beta | 9.2 |
| Secondary Th2 act | 3.3 | HUVEC IFN gamma | 7.2 |
| Secondary Tr1 act | 1.2 | HUVEC TNF alpha + IFN gamma | 4.6 |
| Secondary Th1 rest | 0.3 | HUVEC TNF alpha + IL4 | 5.1 |
| Secondary Th2 rest | 0.2 | HUVEC IL-11 | 4.5 |
| Secondary Tr1 rest | 0.2 | Lung Microvascular EC none | 32.5 |
| Primary Th1 act | 0.5 | Lung Microvascular EC TNF alpha + IL-1beta | 10.3 |
| Primary Th2 act | 0.7 | Microvascular Dermal EC none | 4.2 |
| Primary Tr1 act | 1.0 | Microsvasular Dermal EC | 2.8 |
| | | TNF alpha + IL-1beta | |
| Primary Th1 rest | 0.2 | Bronchial epithelium TNF alpha + IL1beta | 11.5 |
| Primary Th2 rest | 0.3 | Small airway epithelium none | 15.8 |
| Primary Tr1 rest | 0.2 | Small airway epithelium TNF alpha + IL-1beta | 20.2 |
| CD45RA CD4 lymphocyte act | 4.6 | Coronery artery SMC rest | 6.0 |
| CD45RO CD4 lymphocyte act | 1.7 | Coronery artery SMC TNF alpha + IL-1beta | 5.1 |
| CD8 lymphocyte act | 0.3 | Astrocytes rest | 1.5 |
| Secondary CD8 lymphocyte rest | 1.1 | Astrocytes TNF alpha + IL-1beta | 1.9 |
| Secondary CD8 lymphocyte act | 0.3 | KU-812 (Basophil) rest | 1.7 |
| CD4 lymphocyte none | 0.1 | KU-812 (Basophil) | 8.9 |
| | | PMA/ionomycin | |
| 2ry Th1/Th2/Tr1_anti-CD95 | 0.8 | CCD1106 (Keratinocytes) none | 6.8 |
| CH11 | | | |
| LAK cells rest | 39.2 | CCD1106 (Keratinocytes) | 5.0 |
| | | TNF alpha + IL-1beta | |
| LAK cells IL-2 | 0.6 | Liver cirrhosis | 3.8 |
| LAK cells IL-2 + IL-12 | 0.1 | NCI-H292 none | 3.6 |
| LAK cells IL-2 + IFN gamma | 0.3 | NCI-H292 IL-4 | 4.7 |
| LAK cells IL-2 + IL-18 | 0.3 | NCI-H292 IL-9 | 5.4 |
| LAK cells PMA/ionomycin | 54.3 | NCI-H292 IL-13 | 3.3 |
| NK Cells IL-2 rest | 0.6 | NCI-H292 IFN gamma | 2.4 |
| Two Way MLR 3 day | 9.0 | HPAEC none | 3.7 |
| Two Way MLR 5 day | 3.4 | HPAEC TNF alpha + IL-1beta | 27.0 |
| Two Way MLR 7 day | 1.3 | Lung fibroblast none | 10.7 |
| PBMC rest | 0.4 | Lung fibroblast TNF alpha + IL- | 10.4 |
| | | 1beta | |
| PBMC PWM | 0.7 | Lung fibroblast IL-4 | 4.5 |
| PBMC PHA-L | 2.7 | Lung fibroblast IL-9 | 8.2 |
| Ramos (B cell) none | 0.0 | Lung fibroblast IL-13 | 2.2 |
| Ramos (B cell) ionomycin | 0.0 | Lung fibroblast IFN gamma | 16.0 |
| B lymphocytes PWM | 0.5 | Dermal fibroblast CCD1070 rest | 17.6 |
| B lymphocytes CD40L and IL-4 | 1.3 | Dermal fibroblast CCD1070 TNF | 16.6 |
| | | alpha | |
| EOL-1 dbcAMP | 1.0 | Dermal fibroblast CCD1070 IL- | 16.7 |
| | | 1beta | |
| EOL-1 dbcAMP | 0.9 | Dermal fibroblast IFN gamma | 31.6 |
| PMA/ionomycin | | | |
| Dendritic cells none | 100.0 | Dermal fibroblast IL-4 | 20.3 |
| Dendritic cells LPS | 31.9 | Dermal fibroblast rest | 14.6 |
| Dendritic cells anti-CD40 | 36.3 | Neutrophils TNFa + LPS | 0.2 |
| Monocytes rest | 1.4 | Neutrophils rest | 0.2 |
| Monocytes LPS | 40.9 | Colon | 0.0 |
| Macrophages rest | 26.1 | Lung | 1.4 |
| Macrophages LPS | 16.7 | Thymus | 0.2 |
| HUVEC none | 4.7 | Kidney | 9.7 |
| HUVEC starved | 5.8 | | |

CNS_neurodegeneration_v1.0 Summary: Ag5264 This panel confirms the expression of this gene at low levels in the brains of an independent group of individuals. However, no differential expression of this gene was detected between Alzheimer's diseased postmortem brains and those of non-demented controls in this experiment. Please see Panel 1.5 for a discussion of the potential utility of this gene in treatment of central nervous system disorders.

General_screen_panel_v1.5 Summary: Ag5264 Highest expression of this gene is detected in breast cancer BT-549 cell line (CT=25). Moderate levels of expression of this gene is also seen in cluster of cancer cell lines derived from pancreatic, gastric, colon, lung, liver, renal, breast, ovarian, prostate, melanoma and brain cancers. Thus, expression of this gene could be used as a marker to detect the presence of these cancers. Furthermore, therapeutic modulation of the expression or function of this gene may be effective in the treatment of pancreatic, gastric, colon, lung, liver, renal, breast, ovarian, prostate, melanoma and brain cancers.

Among tissues with metabolic or endocrine function, this gene is expressed at moderate levels in pancreas, adipose, adrenal gland, thyroid, pituitary gland, skeletal muscle, heart, liver and the gastrointestinal tract. Therefore, therapeutic modulation of the activity of this gene may prove useful in the treatment of endocrine/metabolically related diseases, such as obesity and diabetes.

In addition, this gene is expressed at moderate levels in all regions of the central nervous system examined, including amygdala, hippocampus, substantia nigra, thalamus, cerebellum, cerebral cortex, and spinal cord. Therefore, therapeutic modulation of this gene product may be useful in the treatment of central nervous system disorders such as Alzheimer's disease, Parkinson's disease, epilepsy, multiple sclerosis, schizophrenia and depression.

Panel 4.1D Summary: Ag5264 Highest levels of expression of this gene is detected in resting dendritic cells (CT=28.7). Moderate to low levels of expression of this gene is also seen in activated dendritic cells, resting and PMA/ionomycin stimulated LAK cells, monocytes, macrophage, different types of endothelial cells, small airway epithelium, lung and dermal fibroblasts and normal tissue represent by lung and kidney. This gene is upregulated in LPS treated monocytes, cytokine treated UPAEC, and activated secondary Th1, Th2 cells. Therefore, therapeutic modulation of this gene or its protein product may alter the functions associated with these cell types and would be beneficial in the treatment of autoimmune and inflammatory diseases such as asthma, allergies, inflammatory bowel disease, lupus erythematosus, psoriasis, rheumatoid arthritis, and osteoarthritis.

AP. CG57284-03: RAS-Related Protein RAB-5C.

Expression of gene CG57284-03 was assessed using the primer-probe set Ag6892, described in Table APA. Results of the RTQ-PCR runs are shown in Tables APB and APC. Please note that this sequence represents a full-length physical clone.

TABLE APA

Probe Name Ag6892

| | | | Start | SEQ ID |
|---|---|---|---|---|
| Primers | | Length | Position | No |
| Forward | 5′-gtgtcatccaggcagacagtct-3′ | 22 | 473 | 414 |
| Probe | TET-5′-ccgctccaattgtgctctcctggtact-3′-TAMRA | 27 | 507 | 415 |
| Reverse | 5′-cgctttqtcaagggacagttt-3′ | 21 | 538 | 416 |

TABLE APB
General_screening_panel_v1.6

| | Rel. | | Rel. |
|---|---|---|---|
| | Exp. (%) | | Exp. (%) |
| | Ag6892, | | Ag6892, |
| | Run | | Run |
| Tissue Name | 278388295 | issue Name | 278388295 |
| Adipose | 11.0 | Renal ca. TK-10 | 41.5 |
| Melanoma* Hs688(A).T | 37.4 | Bladder | 19.1 |
| Melanoma* Hs688(B).T | 33.0 | Gastric ca. (liver met.) NCI-N87 | 26.4 |
| Melanoma* M14 | 85.3 | Gastric ca. KATO III | 93.3 |
| Melanoma* LOXIMVI | 48.6 | Colon ca. SW-948 | 15.7 |
| Melanoma* SK-MEL-5 | 49.7 | Colon ca. SW480 | 62.4 |
| Squamous cell carcinoma SCC-4 | 28.5 | Colon ca.* (SW480 met) SW620 | 9.5 |
| Testis Pool | 10.1 | Colon ca. HT29 | 20.7 |
| Prostate ca.* (bone met) PC-3 | 0.0 | Colon ca. HCT-116 | 48.0 |
| Prostate Pool | 10.6 | Colon ca. CaCo-2 | 49.7 |
| Placenta | 22.4 | Colon cancer tissue | 19.3 |
| Uterus Pool | 4.8 | Colon ca. SW1116 | 6.7 |
| Ovarian ca. OVCAR-3 | 18.9 | Colon ca. Colo-205 | 13.3 |
| Ovarian ca. SK-OV-3 | 63.3 | Colon ca. SW-48 | 16.5 |
| Ovarian ca. OVCAR-4 | 17.4 | Colon Pool | 15.5 |
| Ovarian ca. OVCAR-5 | 41.5 | Small Intestine Pool | 8.7 |
| Ovarian ca. IGROV-1 | 18.4 | Stomach Pool | 8.0 |
| Ovarian ca. OVCAR-8 | 13.8 | Bone Marrow Pool | 8.5 |
| Ovary | 10.6 | Fetal Heart | 5.9 |
| Breast ca. MCF-7 | 33.2 | Heart Pool | 6.3 |
| Breast ca. MDA-MB-231 | 46.0 | Lymph Node Pool | 16.4 |
| Breast ca. BT 549 | 37.4 | Fetal Skeletal Muscle | 5.4 |
| Breast ca. T47D | 35.1 | Skeletal Muscle Pool | 1.6 |
| Breast ca. MDA-N | 22.2 | Spleen Pool | 8.8 |
| Breast Pool | 12.7 | Thymus pool | 8.7 |
| Trachea | 12.0 | CNS cancer (glio/astro) U87-MG | 35.4 |
| Lung | 2.5 | CNS cancer (glio/astro) U-118-MG | 55.9 |
| Fetal Lung | 32.5 | CNS cancer (neuro; met) SK-N-AS | 52.1 |
| Lung ca. NCI-N417 | 5.4 | CNS cancer (astro) SF-539 | 28.9 |
| Lung ca. LX-1 | 20.2 | CNS cancer (astro) SNB-75 | 52.9 |
| Lung ca. NCI-H146 | 8.6 | CNS cancer (glio) SNB-19 | 21.2 |
| Lung ca. SHP-77 | 20.2 | CNS cancer (glio) SF-295 | 100.0 |
| Lung ca. A549 | 51.1 | Brain (Amygdala) Pool | 10.6 |
| Lung ca. NCI-H526 | 5.6 | Brain (cerebellum) | 49.0 |
| Lung ca. NCI-H23 | 23.7 | Brain (fetal) | 25.9 |
| Lung ca. NCI-H460 | 19.1 | Brain (Hippocampus) Pool | 13.0 |
| Lung ca. HOP-62 | 21.0 | Cerebral Cortex Pool | 17.3 |
| Lung ca. NCI-H522 | 31.4 | Brain (Substantia nigra) Pool | 11.2 |
| Liver | 5.7 | Brain (Thalamus) Pool | 19.6 |
| Fetal Liver | 19.8 | Brain (whole) | 23.0 |
| Liver ca. HepG2 | 10.3 | Spinal Cord Pool | 12.5 |
| Kidney Pool | 15.9 | Adrenal Gland | 24.8 |
| Fetal Kidney | 14.0 | Pituitary gland Pool | 2.7 |
| Renal ca. 786-0 | 24.3 | Salivary Gland | 11.3 |
| Renal ca. A498 | 21.9 | Thyroid (female) | 9.8 |
| Renal ca. ACHN | 22.2 | Pancreatic ca. CAPAN2 | 24.8 |
| Renal ca. UO-31 | 35.4 | Pancreas Pool | 8.1 |

TABLE APC
Panel 5 Islet

| | Rel. | | Rel. |
|---|---|---|---|
| | Exp. (%) | | Exp. (%) |
| | Ag6892, | | Ag6892, |
| | Run | | Run |
| Tissue Name | 305424859 | Tissue Name | 305424859 |
| 97457_Patient-02go_adipose | 4.5 | 94709_Donor 2 AM - A_adipose | 44.1 |
| 97476_Patient-07sk_skeletal | 0.0 | 94710_Donor 2 AM - B_adipose | 30.8 |
| muscle | | | |
| 97477_Patient-07ut_uterus | 8.2 | 94711_Donor 2 AM - C_adipose | 21.0 |
| 97478_Patient-07pl_placenta | 13.1 | 94712_Donor 2 AD - A_adipose | 48.0 |
| 99167_Bayer Patient 1 | 23.2 | 94713_Donor 2 AD - B_adipose | 54.0 |
| 97482_Patient-08ut_uterus | 7.7 | 94714_Donor 2 AD - C_adipose | 50.3 |
| 97483_Patient-08pl_placenta | 18.9 | 94742_Donor 3 U - A_Mesenchymal | 14.7 |
| | | Stem Cells | |
| 97486_Patient-09sk_skeletal | 4.4 | 94743_Donor 3 U - B_Mesenchymal | 10.4 |
| muscle | | Stem Cells | |
| 97487_Patient-09ut_uterus | 19.6 | 94730_Donor 3 AM - A_adipose | 53.2 |
| 97488_Patient-09pl_placenta | 11.3 | 94731_Donor 3 AM - B_adipose | 74.2 |
| 97492_Patient-10ut_uterus | 12.2 | 94732_Donor 3 AM - C_adipose | 58.6 |
| 97493_Patient-10pl_placenta | 34.9 | 94733_Donor 3 AD - A_adipose | 64.6 |
| 97495_Patient-11go_adipose | 9.2 | 94734_Donor 3 AD - B_adipose | 100.0 |
| 97496_Patient-11sk_skeletal | 3.8 | 94735_Donor 3 AD - C_adipose | 20.4 |
| muscle | | | |
| 97497_Patient-11ut_uterus | 25.0 | 77138_Liver_HepG2untreated | 71.2 |
| 97498_Patient-11pl_placenta | 8.8 | 73556_Heart_Cardiac stromal cells | 18.6 |
| | | (primary) | |
| 97500_Patient-12go_adipose | 10.4 | 81735_Small Intestine | 12.4 |
| 97501_Patient-12sk_skeletal muscle | 12.7 | 72409_Kidney_Proximal Convoluted | 81.2 |
| muscle | | Tubule | |
| 97502_Patient-12ut_uterus | 18.9 | 82685_Small intestine_Duodenum | 8.1 |
| 97503_Patient-12pl_placenta | 17.8 | 90650_Adrenal_Adrenocortical | 4.8 |
| | | adenoma | |
| 94721_Donor 2 U - | 27.9 | 72410_Kidney_HRCE | 37.9 |
| A_Mesenchymal Stem Cells | | | |
| 94722_Donor 2 U - | 25.7 | 72411_Kidney_HRE | 18.8 |
| B_Mesenchymal Stem Cells | | | |
| 94723_Donor 2 U - | 30.4 | 73139_Uterus_Uterine smooth | 48.0 |
| C_Mesenchymal Stem Cells | | muscle cells | |

General_screen_panel_v1.6 Summary: Ag6892 Highest expression of this gene is seen in a brain cancer cell line (CT=24.1). This gene is ubiquitously expressed in this panel, with high levels of expression seen in brain, colon, gastric, lung, breast, ovarian, and melanoma cancer cell lines. This expression profile suggests a role for this gene product in cell survival and proliferation. Modulation of this gene product may be useful in the treatment of cancer.

Among tissues with metabolic function, this gene is expressed at high levels in pituitary, adipose, adrenal gland, pancreas, thyroid, and adult and fetal skeletal muscle, heart, and liver. This widespread expression among these tissues suggests that this gene product may play a role in normal neuroendocrine and metabolic function and that disregulated expression of this gene may contribute to neuroendocrine disorders or metabolic diseases, such as obesity and diabetes.

This gene is also expressed at high levels in the CNS, including the hippocampus, thalamus, substantia nigra, amygdala, cerebellum and cerebral cortex. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

In addition, this gene is expressed at much higher levels in fetal lung tissue (CT=25.7) when compared to expression in the adult counterpart (CT=29.4). Thus, expression of this gene may be used to differentiate between the fetal and adult source of this tissue.

Panel 5 Islet Summary: Ag6892 Highest expression is seen in adipose (CT=26), with nearly ubiquitous expression seen across the samples on this panel. High to moderate levels of expression are seen in metabolic tissues, including skeletal muscle, adipose, and placenta, in agreement with Panel 1.6. Please see that panel for discussion of this gene in metabolic disease.

AQ. CG57308-02: Sulfonylurea Receptor 1 Splice Variant.

Expression of gene CG57308-02 was assessed using the primer-probe set Ag7558, described in Table AQA. Results of the RTQ-PCR runs are shown in Tables AQB and AQC.

TABLE AQA

Probe Name Ag7558

| | | | Start | SEQ ID |
|---|---|---|---|---|
| Primers | | Length | Position | No |
| Forward | 5′-tcgaagggcacatcatca-3′ | 18 | 4319 | 417 |
| Probe | TET-5′-tgcctctgtccctggctgaaattctc-3′-TAMRA | 26 | 4348 | 418 |
| Reverse | 5′-tgaagatgctggtcttcctca-3′ | 21 | 4400 | 419 |

TABLE AQB
CNS_neurodegeneration_v1.0

| | Rel. | | Rel. |
|---|---|---|---|
| | Exp. (%) | | Exp. (%) |
| | Ag7558, | | Ag7558, |
| | Run | | Run |
| Tissue Name | 308750599 | issue Name | 308750599 |
| AD 1 Hippo | 4.2 | Control (Path) 3 Temporal Ctx | 3.3 |
| AD 2 Hippo | 16.4 | Control (Path) 4 Temporal Ctx | 50.3 |
| AD 3 Hippo | 1.7 | AD 1 Occipital Ctx | 11.1 |
| AD 4 Hippo | 11.3 | AD 2 Occipital Ctx (Missing) | 0.0 |
| AD 5 Hippo | 76.3 | AD 3 Occipital Ctx | 2.3 |
| AD 6 Hippo | 38.7 | AD 4 Occipital Ctx | 19.8 |
| Control 2 Hippo | 17.8 | AD 5 Occipital Ctx | 45.4 |
| Control 4 Hippo | 3.9 | AD 6 Occipital Ctx | 21.2 |
| Control (Path) 3 Hippo | 1.0 | Control 1 Occipital Ctx | 0.9 |
| AD 1 Temporal Ctx | 7.6 | Control 2 Occipital Ctx | 82.4 |
| AD 2 Temporal Ctx | 24.5 | Control 3 Occipital Ctx | 13.4 |
| AD 3 Temporal Ctx | 4.0 | Control 4 Occipital Ctx | 0.0 |
| AD 4 Temporal Ctx | 32.3 | Control (Path) 1 Occipital Ctx | 100.0 |
| AD 5 Inf Temporal Ctx | 78.5 | Control (Path) 2 Occipital Ctx | 17.1 |
| AD 5 Sup Temporal Ctx | 25.3 | Control (Path) 3 Occipital Ctx | 0.0 |
| AD 6 Inf Temporal Ctx | 39.2 | Control (Path) 4 Occipital Ctx | 31.9 |
| AD 6 Sup Temporal Ctx | 71.7 | Control 1 Parietal Ctx | 1.8 |
| Control 1 Temporal Ctx | 4.3 | Control 2 Parietal Ctx | 36.9 |
| Control 2 Temporal Ctx | 33.2 | Control 3 Parietal Ctx | 21.5 |
| Control 3 Temporal Ctx | 13.8 | Control (Path) 1 Parietal Ctx | 87.1 |
| Control 3 Temporal Ctx | 2.5 | Control (Path) 2 Parietal Ctx | 41.5 |
| Control (Path) 1 Temporal Ctx | 55.9 | Control (Path) 3 Parietal Ctx | 3.7 |
| Control (Path) 2 Temporal Ctx | 65.1 | Control (Path) 4 Parietal Ctx | 79.0 |

TABLE AQC
Panel 5 Islet

| | Rel. | | Rel. |
|---|---|---|---|
| | Exp. (%) | | Exp. (%) |
| | Ag7558, | | Ag7558, |
| | Run | | Run |
| Tissue Name | 312000203 | Tissue Name | 312000203 |
| 97457_Patient-02go_adipose | 0.0 | 94709_Donor 2 AM - A_adipose | 0.0 |
| 97476_Patient-07sk_skeletal | 0.0 | 94710_Donor 2 AM - B_adipose | 0.0 |
| muscle | | | |
| 97477_Patient-07ut_uterus | 0.0 | 94711_Donor 2 AM - C_adipose | 0.0 |
| 97478_Patient-07pl_placenta | 0.0 | 94712_Donor 2 AD - A_adipose | 0.0 |
| 99167_Bayer Patient 1 | 100.0 | 94713_Donor 2 AD - B_adipose | 0.0 |
| 97482_Patient-08ut_uterus | 0.0 | 94714_Donor 2 AD - C_adipose | 0.0 |
| 97483_Patient-08pl_placenta | 0.0 | 94742_Donor 3 U - A_Mesenchymal | 0.0 |
| | | Stem Cells | |
| 97486_Patient-09sk_skeletal | 0.0 | 94743_Donor 3 U - B_Mesenchymal | 0.0 |
| muscle | | Stem Cells | |
| 97487_Patient-09ut_uterus | 0.0 | 94730_Donor 3 AM - A_adipose | 0.0 |
| 97488_Patient-09pl_placenta | 0.0 | 94731_Donor 3 AM - B_adipose | 0.0 |
| 97492_Patient-10ut_uterus | 0.0 | 94732_Donor 3 AM - C_adipose | 0.0 |
| 97493_Patient-10pl_placenta | 0.0 | 94733_Donor 3 AD - A_adipose | 0.0 |
| 97495_Patient-11go_adipose | 0.0 | 94734_Donor 3 AD - B_adipose | 0.0 |
| 97496_Patient-11sk_skeletal | 0.0 | 94735_Donor 3 AD - C_adipose | 0.0 |
| muscle | | | |
| 97497_Patient-11ut_uterus | 0.0 | 77138_Liver_HepG2untreated | 0.0 |
| 97498_Patient-11pl_placenta | 0.0 | 73556_Heart_Cardiac stromal cells | 0.0 |
| | | (primary) | |
| 97500_Patient-12go_adipose | 0.0 | 81735_Small Intestine | 0.0 |
| 97501_Patient-12sk_skeletal | 0.0 | 72409_Kidney_Proximal Convoluted | 0.0 |
| muscle | | Tubule | |
| 97502_Patient-12ut_uterus | 0.0 | 82685_Small intestine_Duodenum | 0.0 |
| 97503_Patient-12pl_placenta | 0.0 | 90650_Adrenal_Adrenocortical | 0.0 |
| | | adenoma | |
| 94721_Donor 2 U - | 0.0 | 72410_Kidney_HRCE | 0.0 |
| A_Mesenchymal Stem Cells | | | |
| 94722_Donor 2 U - | 0.0 | 72411_Kidney_HRE | 0.0 |
| B_Mesenchymal Stem Cells | | | |
| 94723_Donor 2 U - | 0.0 | 73139_Uterus_Uterine smooth | 0.0 |
| C_Mesenchymal Stem Cells | | muscle cells | |

CNS_neurodegeneration_v1.0 Summary: Ag7558 Highest expression of this gene is seen in the occipital cortex of a control patient (CT=33). This panel does not show differential expression of this gene in Alzheimer's disease. However, this profile does show the expression of this gene at low levels in the brain. Therefore, therapeutic modulation of the expression or function of this gene may be useful in the treatment of neurological disorders, such as Alzheimer's disease, Parkinson's disease, schizophrenia, multiple sclerosis, stroke and epilepsy.

Panel 4.1D Summary: Ag7558 Expression of this gene is low/undetectable in all samples on this panel (CTs>35).

Panel 5 Islet Summary: Ag7558 Expression of this gene is limited to pancreatic islet cells (CT=34.6). This gene codes for a variant of SURI. SURI is a subunit of the pancreatic beta cell K+ channel that regulates insulin release in glucose-stimulated cells. Thus, therapeutic modulation of SURI variant encoded by this gene may be used as a treatment for the enhancement of insulin secretion in Type 2 diabetes.

AR. CG93659-03: Mirogen-Activated Protein Kinase Kinase Kinase 9.

Expression of gene CG93659-03 was assessed using the primer-probe set Ag4828, described in Table ARA. Results of the RTQ-PCR runs are shown in Tables ARB and ARC.

TABLE ARA

Probe Name Ag4828

| | | | Start | SEQ ID |
|---|---|---|---|---|
| Primers | | Length | Position | No |
| Forward | 5′-gaggaatctgagatgctcaaga-3′ | 22 | 1275 | 420 |
| Probe | TET-5′-caacgctctctctacatcgacctcgg-3′-TAMRA | 26 | 1299 | 421 |
| Reverse | 5′-tccccgaacaagattgaagt-3′ | 20 | 1339 | 422 |

TABLE ARB
General_screening_panel_v1.4

| | Rel. | | Rel. |
|---|---|---|---|
| | Exp. (%) | | Exp. (%) |
| | Ag4828, | | Ag4828, |
| | Run | | Run |
| Tissue Name | 217081802 | issue Name | 217081802 |
| Adipose | 53.6 | Renal ca. TK-10 | 10.6 |
| Melanoma* Hs688(A).T | 15.5 | Bladder | 31.9 |
| Melanoma* Hs688(B).T | 17.4 | Gastric ca. (liver met.) NCI-N87 | 36.3 |
| Melanoma* M14 | 3.5 | Gastric ca. KATO III | 12.2 |
| Melanoma* LOXIMVI | 3.2 | Colon ca. SW-948 | 5.4 |
| Melanoma* SK-MEL-5 | 0.9 | Colon ca. SW480 | 25.0 |
| Squamous cell carcinoma SCC-4 | 7.0 | Colon ca.* (SW480 met) SW620 | 2.5 |
| Testis Pool | 4.7 | Colon ca. HT29 | 14.3 |
| Prostate ca.* (bone met) PC-3 | 6.3 | Colon ca. HCT-116 | 2.1 |
| Prostate Pool | 3.9 | Colon ca. CaCo-2 | 15.9 |
| Placenta | 39.0 | Colon cancer tissue | 39.8 |
| Uterus Pool | 9.0 | Colon ca. SW1116 | 3.4 |
| Ovarian ca. OVCAR-3 | 15.7 | Colon ca. Colo-205 | 8.8 |
| Ovarian ca. SK-OV-3 | 46.3 | Colon ca. SW-48 | 5.4 |
| Ovarian ca. OVCAR-4 | 7.1 | Colon Pool | 16.2 |
| Ovarian ca. OVCAR-5 | 30.6 | Small Intestine Pool | 9.3 |
| Ovarian ca. IGROV-1 | 14.1 | Stomach Pool | 17.3 |
| Ovarian ca. OVCAR-8 | 2.7 | Bone Marrow Pool | 7.0 |
| Ovary | 4.5 | Fetal Heart | 2.9 |
| Breast ca. MCF-7 | 100.0 | Heart Pool | 7.9 |
| Breast ca. MDA-MB-231 | 9.2 | Lymph Node Pool | 15.2 |
| Breast ca. BT 549 | 73.2 | Fetal Skeletal Muscle | 1.7 |
| Breast ca. T47D | 66.0 | Skeletal Muscle Pool | 9.8 |
| Breast ca. MDA-N | 0.9 | Spleen Pool | 45.7 |
| Breast Pool | 24.1 | Thymus Pool | 15.9 |
| Trachea | 18.0 | CNS cancer (glio/astro) U87-MG | 7.6 |
| Lung | 6.7 | CNS cancer (glio/astro) U-118-MG | 7.9 |
| Fetal Lung | 68.3 | CNS cancer (neuro; met) SK-N-AS | 2.6 |
| Lung ca. NCI-N417 | 0.2 | CNS cancer (astro) SF-539 | 2.3 |
| Lung ca. LX-1 | 11.8 | CNS cancer (astro) SNB-75 | 14.1 |
| Lung ca. NCI-H146 | 0.0 | CNS cancer (glio) SNB-19 | 11.1 |
| Lung ca. SHP-77 | 0.1 | CNS cancer (glio) SF-295 | 31.9 |
| Lung ca. A549 | 36.6 | Brain (Amygdala) Pool | 2.7 |
| Lung ca. NCI-H526 | 0.0 | Brain (cerebellum) | 1.4 |
| Lung ca. NCI-H23 | 13.4 | Brain (fetal) | 4.9 |
| Lung ca. NCI-H460 | 17.6 | Brain (Hippocampus) Pool | 3.7 |
| Lung ca. HOP-62 | 13.2 | Cerebral Cortex Pool | 3.5 |
| Lung ca. NCI-H522 | 2.1 | Brain (Substantia nigra) Pool | 2.7 |
| Liver | 1.0 | Brain (Thalamus) Pool | 4.5 |
| Fetal Liver | 2.8 | Brain (whole) | 4.5 |
| Liver ca. HepG2 | 8.1 | Spinal Cord Pool | 3.8 |
| Kidney Pool | 31.4 | Adrenal Gland | 9.5 |
| Fetal Kidney | 7.7 | Pituitary gland Pool | 1.4 |
| Renal ca. 786-0 | 10.9 | Salivary Gland | 2.5 |
| Renal ca. A498 | 5.2 | Thyroid (female) | 7.7 |
| Renal ca. ACHN | 2.5 | Pancreatic ca. CAPAN2 | 34.4 |
| Renal ca. UO-31 | 14.9 | Pancreas Pool | 19.6 |

TABLE ARC
Panel 5D

| | Rel. | | Rel. |
|---|---|---|---|
| | Exp. %) | | Exp. (%) |
| | Ag4828, | | Ag4828, |
| | Run | | Run |
| Tissue Name | 219436967 | Tissue Name | 219436967 |
| 97457_Patient-02go_adipose | 33.9 | 94709_Donor 2 AM - A_adipose | 10.8 |
| 97476_Patient-07sk_skeletal | 33.4 | 94710_Donor 2 AM - B_adipose | 9.3 |
| muscle | | | |
| 97477_Patient-07ut_uterus | 59.5 | 94711_Donor 2 AM - C_adipose | 3.0 |
| 97478_Patient-07pl_placenta | 39.8 | 94712_Donor 2 AD - A_adipose | 13.7 |
| 97481_Patient-08sk_skeletal | 25.9 | 94713_Donor 2 AD - B_adipose | 10.0 |
| muscle | | | |
| 97482_Patient-08ut_uterus | 19.8 | 94714_Donor 2 AD - C_adipose | 6.7 |
| 97483_Patient-08pl_placenta | 41.5 | 94742_Donor 3 U - A_Mesenchymal | 4.7 |
| | | Stem Cells | |
| 97486_Patient-09sk_skeletal | 6.5 | 94743_Donor 3 U - B_Mesenchymal | 2.8 |
| muscle | | Stem Cells | |
| 97487_Patient-09ut_uterus | 8.1 | 94730_Donor 3 AM - A_adipose | 6.3 |
| 97488_Patient-09pl_placenta | 38.4 | 94731_Donor 3 AM - B_adipose | 2.4 |
| 97492_Patient-10ut_uterus | 30.6 | 94732_Donor 3 AM - C_adipose | 2.2 |
| 97493_Patient-10pl_placenta | 72.7 | 94733_Donor 3 AD - A_adipose | 10.2 |
| 97495_Patient-11go_adipose | 100.0 | 94734_Donor 3 AD - B_adipose | 5.5 |
| 97496_Patient-11sk_skeletal | 5.8 | 94735_Donor 3 AD - C_adipose | 4.7 |
| muscle | | | |
| 97497_Patient-11ut_uterus | 20.6 | 77138_Liver_HepG2untreated | 14.4 |
| 97498_Patient-11pl_placenta | 50.0 | 73556_Heart_Cardiac stromal cells | 1.9 |
| | | (primary) | |
| 97500_Patient-12go_adipose | 82.4 | 81735_Small Intestine | 17.2 |
| 97501_Patient-12sk_skeletal | 19.2 | 72409_Kidney_Proximal Convoluted | 0.9 |
| muscle | | Tubule | |
| 97502_Patient-12ut_uterus | 23.7 | 82685_Small intestine_Duodenum | 19.1 |
| 97503_Patient-12pl_placenta | 57.0 | 90650_Adrenal_Adrenocortical | 8.8 |
| | | adenoma | |
| 94721_Donor 2 U - | 1.6 | 72410_Kidney_HRCE | 7.6 |
| A_Mesenchymal Stem Cells | | | |
| 94722_Donor 2 U - | 3.0 | 72411_Kidney_HRE | 13.5 |
| B_Mesenchymal Stem Cells | | | |
| 94723_Donor 2 U - | 2.1 | 73139_Uterus_Uterine smooth | 2.0 |
| C_Mesenchymal Stem Cells | | muscle cells | |

General_screen_panel_v1.4 Summary: Ag4828 Highest expression of this gene is detected in a breast cancer MCF-7 cell line(CT=27.6). Interestingly, this gene is expressed at much higher levels in fetal (CT=28) when compared to adult lung (CT=31).

This observation suggests that expression of this gene can be used to distinguish fetal from adult lung. In addition, the relative overexpression of this gene in fetal lung suggests that the protein product may enhance lung growth or development in the fetus and thus may also act in a regenerative capacity in the adult. Therefore, therapeutic modulation of the protein encoded by this gene could be useful in treatment of lung related diseases.

In addition significant expression of this gene is found in a number of cancer (pancreatic, CNS, colon, lung, breast, ovary, prostate, melanoma) cell lines. Therefore, therapeutic modulation of the activity of this gene or its protein product, through the use of small molecule drugs, might be beneficial in the treatment of these cancers.

Among tissues with metabolic or endocrine function, this gene is expressed at high to moderate levels in pancreas, adipose, adrenal gland, thyroid, skeletal muscle, heart, fetal liver and the gastrointestinal tract. Therefore, therapeutic modulation of the activity of this gene may prove useful in the treatment of endocrine/metabolically related diseases, such as obesity and diabetes.

This gene encodes a protein that is homologous to mitogen-activated protein kinase kinase kinase 8 (MAP3K8)(COT proto-oncogene serine/threonine-protein kinase) (C-COT) (Cancer osaka thyroid oncogene). COT is able to enhance the TNF alpha production and to activate NF-kB. Both events are connected with insulin resistance and type II diabetes (1, 2, 3). Inhibition of COT kinase would prevent overproduction of TNF alpha and activation of NF_{kB}, thus improving insulin resistance and diabetes.

In addition, this gene is expressed at high levels in all regions of the central nervous system examined, including amygdala, hippocampus, substantia nigra, thalamus, cerebellum, cerebral cortex, and spinal cord. Recently, MKK6, a related protein, has been shown to associated with Alzheimer's disease (4). Therefore, based on the homology of this protein to MKK6 and the presence of this gene in the brain, we predict that this putative MAP3K8 may play a role in central nervous system disorders such as Alzheimer's disease, Parkinson's disease, epilepsy, multiple sclerosis, schizophrenia and depression.

References:

1. Ballester A, Velasco A, Tobena R, Alemany S. Cot kinase activates tumor necrosis factor-alpha gene expression in a cyclosporin A-resistant manner. J. Biol. Chem. 1998. 273, 14099-106. PMID: 9603908.

2. Bierhaus A, Schiekofer S, Schwaninger M, Andrassy M, Humpert P M, Chen J, Hong M, Luther T, Henle T, Kloting I, Morcos M, Hofmann M, Tritschler H, Weigle B, Kasper M, Smith M, Perry G, Schmidt A M, Stern D M, Haring H U, Schleicher E, Nawroth P P. Diabetes-associated sustained activation of the transcription factor nuclear factor-kappaB. Diabetes, 2001 50, 2792-808. PMID: 11723063.

3. Belich M P, Salmeron A, Johnston L H, Ley S C. TPL-2 kinase regulates the proteolysis of the NF-kappaB-inhibitory protein NF-kappaB1 p105. Nature. 1999 397, 363-8. PMID: 9950430.

4. Zhu X, Rottkamp C A, Hartzler A, Sun Z, Takeda A, Boux H, Shimohama S, Perry G, Smith M A. (2001) Activation of MKK6, an upstream activator of p38, in Alzheimer's disease. J Neurochem 79(2):311-8

Panel 5D Summary: Ag4828 Highest expression of this gene is detected in adipose tissue (CT=29). Low to moderate expression of this gene is seen in wide range of samples used in this panel including adipose, skeletal muscle, uterus, and placenta. This wide spread expression of this gene in tissues with metabolic or endocrine function, suggests that this gene plays a role in endocrine/metabolically related diseases, such as obesity and diabetes.

This gene encodes a MAP3K8-like protein. Recently, activation of MAP kinase, ERK, a related protein, by modified LDL in vascular smooth muscle cells has been implicated in the development of atherosclerosis in diabetes (Ref. 1). Therefore, this putative MAP3K8 may also play a role in the development of this disease. Therefore, therapeutic modulation of the activity of this gene or its protein product, through the use of small molecule drugs, might be beneficial in the treatment of artheroscierosis and diabetes.

References:

1. Velarde V, Jenkins A J, Christopher J, Lyons T J, Jaffa A A. (2001) Activation of MAPK by modified low-density lipoproteins in vascular smooth muscle cells. J Appl Physiol 91(3):1412-20

AS. CG94521-02 and CG94521-03: Cytoplasmic Glycerol-3-Phosphate Dehydrogenase [NAD+].

Expression of gene CG94521-02 and CG94521-03 was assessed using the primer-probe set Ag3924, described in Table ASA. Results of the RTQ-PCR runs are shown in Tables ASB, ASC, ASD, ASE and ASF. Please note that these sequences represent full-length physical clones.

TABLE ASA

Probe Name Ag3924

| | | | Start | SEQ ID |
|---|---|---|---|---|
| Primers | | Length | Position | No |
| Forward | 5′-actgggaagaccattgaagagt-3′ | 22 | 197 | 423 |
| Probe | TET-5′-aaaagctccaaggaccgcagacttct-3′-TAMRA | 26 | 147 | 424 |
| Reverse | 5′-gtttgaggatgcggtacactt-3′ | 21 | 122 | 425 |

TABLE ASB
CNS_neurodegeneration_v1.0

| | Rel. | | Rel. |
|---|---|---|---|
| | Exp. (%) | | Exp. (%) |
| | Ag3924, | | Ag3924, |
| | Run | | Run |
| Tissue Name | 212343350 | issue Name | 212343350 |
| AD 1 Hippo | 8.4 | Control (Path) 3 Temporal Ctx | 6.0 |
| AD 2 Hippo | 21.9 | Control (Path) 4 Temporal Ctx | 2.8 |
| AD 3 Hippo | 8.4 | AD 1 Occipital Ctx | 14.4 |
| AD 4 Hippo | 7.5 | AD 2 Occipital Ctx (Missing) | 0.0 |
| AD 5 hippo | 92.7 | AD 3 Occipital Ctx | 4.8 |
| AD 6 Hippo | 24.5 | AD 4 Occipital Ctx | 14.0 |
| Control 2 Hippo | 25.7 | AD 5 Occipital Ctx | 14.0 |
| Control 4 Hippo | 7.3 | AD 6 Occipital Ctx | 55.5 |
| Control (Path) 3 Hippo | 8.8 | Control 1 Occipital Ctx | 6.1 |
| AD 1 Temporal Ctx | 8.3 | Control 2 Occipital Ctx | 47.3 |
| AD 2 Temporal Ctx | 23.8 | Control 3 Occipital Ctx | 9.8 |
| AD 3 Temporal Ctx | 4.2 | Control 4 Occipital Ctx | 4.5 |
| AD 4 Temporal Ctx | 15.1 | Control (Path) 1 Occipital Ctx | 64.6 |
| AD 5 Inf Temporal Ctx | 100.0 | Control (Path) 2 Occipital Ctx | 8.6 |
| AD 5 SupTemporal Ctx | 32.3 | Control (Path) 3 Occipital Ctx | 3.9 |
| AD 6 Inf Temporal Ctx | 39.0 | Control (Path) 4 Occipital Ctx | 15.8 |
| AD 6 Sup Temporal Ctx | 33.2 | Control 1 Parietal Ctx | 5.0 |
| Control 1 Temporal Ctx | 4.5 | Control 2 Parietal Ctx | 40.3 |
| Control 2 Temporal Ctx | 44.4 | Control 3 Parietal Ctx | 14.6 |
| Control 3 Temporal Ctx | 11.1 | Control (Path) 1 Parietal Ctx | 70.7 |
| Control 4 Temporal Ctx | 4.4 | Control (Path) 2 Parietal Ctx | 15.5 |
| Control (Path) 1 Temporal Ctx | 49.0 | Control (Path) 3 Parietal Ctx | 4.9 |
| Control (Path) 2 Temporal Ctx | 29.9 | Control (Path) 4 Parietal Ctx | 39.5 |

TABLE ASC
General_screening_panel_v1.4

| | Rel. | | Rel. |
|---|---|---|---|
| | Exp. (%) | | Exp. (%) |
| | Ag3924, | | Ag3924, |
| | Run | | Run |
| Tissue Name | 219515221 | issue Name | 219515221 |
| Adipose | 14.0 | Renal ca. TK-10 | 7.1 |
| Melanoma* Hs688(A).T | 3.6 | Bladder | 8.1 |
| Melanoma* Hs688(B).T | 4.9 | Gastric ca. (liver met.) NCI-N87 | 7.7 |
| Melanoma* M14 | 15.1 | Gastric ca. KATO III | 17.4 |
| Melanoma* LOXIMVI | 6.2 | Colon ca. SW-948 | 25.5 |
| Melanoma* SK-MEL-5 | 37.6 | Colon ca. SW480 | 28.3 |
| Squamous cell carcinoma SCC-4 | 1.1 | Colon ca.* (SW480 met) SW620 | 6.6 |
| Testis Pool | 6.3 | Colon ca. HT29 | 4.1 |
| Prostate ca.* (bone met) PC-3 | 47.0 | Colon ca. HCT-116 | 25.0 |
| Prostate Pool | 18.6 | Colon ca. CaCo-2 | 6.9 |
| Placenta | 6.3 | Colon cancer tissue | 7.6 |
| Uterus Pool | 5.1 | Colon ca. SW1116 | 5.2 |
| Ovarian ca. OVCAR-3 | 11.3 | Colon ca. Colo-205 | 2.6 |
| Ovarian ca. SK-OV-3 | 6.8 | Colon ca. SW-48 | 4.4 |
| Ovarian ca. OVCAR-4 | 12.2 | Colon Pool | 9.9 |
| Ovarian ca. OVCAR-5 | 17.9 | Small Intestine Pool | 9.3 |
| Ovarian ca. IGROV-1 | 8.2 | Stomach Pool | 5.2 |
| Ovarian ca. OVCAR-8 | 3.5 | Bone Marrow Pool | 4.9 |
| Ovary | 9.6 | Fetal Heart | 26.1 |
| Breast ca. MCF-7 | 100.0 | Heart Pool | 23.7 |
| Breast ca. MDA-MB-231 | 11.4 | Lymph Node Pool | 8.7 |
| Breast ca. BT 549 | 11.4 | Fetal Skeletal Muscle | 11.2 |
| Breast ca. T47D | 40.9 | Skeletal Muscle Pool | 62.0 |
| Breast ca. MDA-N | 11.7 | Spleen Pool | 9.7 |
| Breast Pool | 8.3 | Thymus Pool | 5.8 |
| Trachea | 15.4 | CNS cancer (glio/astro) U87-MG | 18.2 |
| Lung | 2.8 | CNS cancer (glio/astro) U-118-MG | 11.3 |
| Fetal Lung | 21.8 | CNS cancer (neuro; met) SK-N-AS | 6.6 |
| Lung ca. NCI-N417 | 13.4 | CNS cancer (astro) SF-539 | 4.0 |
| Lung ca. LX-1 | 8.2 | CNS cancer (astro) SNB-75 | 21.9 |
| Lung ca. NCI-H146 | 4.5 | CNS cancer (glio) SNB-19 | 7.6 |
| Lung ca. SHP-77 | 13.3 | CNS cancer (glio) SF-295 | 24.0 |
| Lung ca. A549 | 16.6 | Brain (Amygdala) Pool | 11.4 |
| Lung ca. NCI-H526 | 2.4 | Brain (cerebellum) | 10.2 |
| Lung ca. NCI-H23 | 2.0 | Brain (fetal) | 27.2 |
| Lung ca. NCI-H460 | 2.9 | Brain (Hippocampus) Pool | 11.6 |
| Lung ca. HOP-62 | 6.6 | Cerebral Cortex Pool | 17.2 |
| Lung ca. NCI-H522 | 14.3 | Brain (Substantia nigra) Pool | 10.4 |
| Liver | 0.3 | Brain (Thalamus) Pool | 18.9 |
| Fetal Liver | 1.1 | Brain (whole) | 17.7 |
| Liver ca. HepG2 | 3.4 | Spinal Cord Pool | 14.3 |
| Kidney Pool | 26.4 | Adrenal Gland | 37.9 |
| Fetal Kidney | 6.7 | Pituitary gland Pool | 5.0 |
| Renal ca. 786-0 | 3.0 | Salivary Gland | 11.1 |
| Renal ca. A498 | 1.4 | Thyroid (female) | 17.0 |
| Renal ca. ACHN | 2.5 | Pancreatic ca. CAPAN2 | 2.8 |
| Renal ca. UO-31 | 10.1 | Pancreas Pool | 13.3 |

TABLE ASD
Panel 4.1D

| | Rel. | | Rel. |
|---|---|---|---|
| | Exp. (% | | Exp. (%) |
| | Ag3924, | | Ag3924, |
| | Run | | Run |
| Tissue Name | 170552351 | Tissue Name | 170552351 |
| Secondary Th1 act | 33.9 | HUVEC IL-1beta | 19.6 |
| Secondary Th2 act | 35.4 | HUVEC IFN gamma | 32.3 |
| Secondary Tr1 act | 29.3 | HUVEC TNF alpha + IFN gamma | 8.6 |
| Secondary Th1 rest | 14.8 | HUVEC TNF alpha + IL4 | 19.1 |
| Secondary Th2 rest | 23.7 | HUVEC IL-11 | 17.2 |
| Secondary Tr1 rest | 15.8 | Lung Microvascular EC none | 16.8 |
| Primary Th1 act | 31.0 | Lung Microvascular EC TNF alpha + IL-1beta | 11.0 |
| Primary Th2 act | 33.7 | Microvascular Dermal EC none | 27.7 |
| Primary Tr1 act | 33.9 | Microsvasular Dermal EC | 8.6 |
| | | TNF alpha + IL-1beta | |
| Primary Th1 rest | 27.4 | Bronchial epithelium TNF alpha + IL1beta | 6.7 |
| Primary Th2 rest | 15.3 | Small airway epithelium none | 4.7 |
| Primary Tr1 rest | 34.2 | Small airway epithelium TNF alpha + IL-1beta | 4.0 |
| CD45RA CD4 lymphocyte act | 17.4 | Coronery artery SMC rest | 8.1 |
| CD45RO CD4 lymphocyte act | 28.3 | Coronery artery SMC TNF alpha + IL-1beta | 4.4 |
| CD8 lymphocyte act | 24.1 | Astrocytes rest | 16.4 |
| Secondary CD8 lymphocyte rest | 18.2 | Astrocytes TNF alpha + IL-1beta | 11.9 |
| Secondary CD8 lymphocyte act | 15.2 | KU-812 (Basophil) rest | 37.1 |
| CD4 lymphocyte none | 12.8 | KU-812 (Basophil) | 35.6 |
| | | PMA/ionomycin | |
| 2ry Th1/Th2/Tr1_anti-CD95 | 21.0 | CCD1106 (Keratinocytes) none | 9.5 |
| CH11 | | | |
| LAK cells rest | 17.8 | CCD1106 (Keratinocytes) | 4.8 |
| | | TNF alpha + IL-1beta | |
| LAK cells IL-2 | 26.6 | Liver cirrhosis | 14.4 |
| LAK cells IL-2 + IL-12 | 17.8 | NCI-H292 none | 42.9 |
| LAK cells IL-2 + IFN gamma | 17.8 | NCI-H292 IL-4 | 57.0 |
| LAK cells IL-2 + IL-18 | 32.5 | NCI-H292 IL-9 | 81.2 |
| LAK cells PMA/ionomycin | 7.9 | NCI-H292 IL-13 | 60.7 |
| NK Cells IL-2 rest | 35.6 | NCI-H292 IFN gamma | 39.0 |
| Two Way MLR 3 day | 17.3 | HPAEC none | 21.2 |
| Two Way MLR 5 day | 17.1 | HPAEC TNF alpha + IL-1beta | 13.4 |
| Two Way MLR 7 day | 100.0 | Lung fibroblast none | 18.0 |
| PBMC rest | 15.6 | Lung fibroblast TNF alpha + IL- | 6.0 |
| | | 1beta | |
| PBMC PWM | 16.5 | Lung fibroblast IL-4 | 19.5 |
| PBMC PHA-L | 13.8 | Lung fibroblast IL-9 | 30.8 |
| Ramos (B cell) none | 64.6 | Lung fibroblast IL-13 | 22.2 |
| Ramos (B cell) ionomycin | 70.2 | Lung fibroblast IFN gamma | 20.0 |
| B lymphocytes PWM | 23.8 | Dermal fibroblast CCD1070 rest | 12.5 |
| B lymphocytes CD40L and IL-4 | 17.0 | Dermal fibroblast CCD1070 TNF | 30.1 |
| | | alpha | |
| EOL-1 dbcAMP | 10.8 | Dermal fibroblast CCD1070 IL- | 5.4 |
| | | 1beta | |
| EOL-1 dbcAMP | 2.2 | Dermal fibroblast IFN gamma | 8.2 |
| PMA/ionomycin | | | |
| Dendritic cells none | 13.6 | Dermal fibroblast IL-4 | 17.8 |
| Dendritic cells LPS | 4.5 | Dermal Fibroblast rest | 20.0 |
| Dendritic cells anti-CD40 | 21.6 | Neutrophils TNFa + LPS | 2.1 |
| Monocytes rest | 19.8 | Neutrophils rest | 3.6 |
| Monocytes LPS | 3.0 | Colon | 35.6 |
| Macrophages rest | 14.9 | Lung | 27.7 |
| Macrophages LPS | 1.7 | Thymus | 27.7 |
| HUVEC none | 16.7 | Kidney | 66.4 |
| HUVEC starved | 17.7 | | |

TABLE ASE
Panel 5 Islet

| | Rel. | | Rel. |
|---|---|---|---|
| | Exp. (% | | Exp. (%) |
| | Ag3924, | | Ag3924, |
| | Run | | Run |
| Tissue Name | 268363571 | Tissue Name | 268363571 |
| 97457_Patient-02go_adipose | 18.2 | 94709_Donor 2 AM - A_adipose | 19.6 |
| 97476_Patient-07sk_skeletal | 10.6 | 94710_Donor 2 AM - B_adipose | 13.3 |
| muscle | | | |
| 97477_Patient-07ut_uterus | 10.2 | 94711_Donor 2 AM - C_adipose | 11.0 |
| 97478_Patient-07pl_placenta | 17.0 | 94712_Donor 2 AD - A_adipose | 9.5 |
| 99167_Bayer Patient 1 | 6.5 | 94713_Donor 2 AD - B_adipose | 21.9 |
| 97482_Patient-08ut_uterus | 6.8 | 94714_Donor 2 AD - C_adipose | 16.7 |
| 97483_Patient-08pl_placenta | 11.7 | 94742_Donor 3 U - A_Mesenchymal | 1.8 |
| | | Stem Cells | |
| 97486_Patient-09sk_skeletal | 10.6 | 94743_Donor 3 U - B_Mesenchymal | 1.7 |
| muscle | | Stem Cells | |
| 97487_Patient-09ut_uterus | 12.0 | 94730_Donor 3 AM - A_adipose | 19.6 |
| 97488_Patient-09pl_placenta | 15.4 | 94731_Donor 3 AM - B_adipose | 12.5 |
| 97492_Patient-10ut_uterus | 12.9 | 94732_Donor 3 AM - C_adipose | 12.2 |
| 97493_Patient-10pl_placenta | 29.5 | 94733_Donor 3 AD - A_adipose | 10.2 |
| 97495_Patient-11go_adipose | 17.9 | 94734_Donor 3 AD - B_adipose | 9.2 |
| 97496_Patient-11sk_skeletal | 70.7 | 94735_Donor 3 AD - C_adipose | 8.9 |
| muscle | | | |
| 97497_Patient-11ut_uterus | 18.8 | 77138_Liver_HepG2untreated | 11.1 |
| 97498_Patient-11pl_placenta | 10.3 | 73556_Heart_Cardiac stromal cells | 5.2 |
| | | (primary) | |
| 97500_Patient-12go_adipose | 31.9 | 81735_Small Intestine | 15.9 |
| 97501_Patient-12sk_skeletal | 100.0 | 72409_Kidney_Proximal Convoluted | 6.5 |
| muscle | | Tubule | |
| 97502_Patient-12ut_uterus | 23.8 | 82685_Small intestine_Duodenum | 17.0 |
| 97503_Patient-12pl_placenta | 8.7 | 90650_Adrenal_Adrenocortical | 14.4 |
| | | adenoma | |
| 94721_Donor 2 U - | 3.9 | 72410_Kidney_HRCE | 11.5 |
| A_Mesenchymal Stem Cells | | | |
| 94722_Donor 2 U - | 2.8 | 72411_Kidney_HRE | 3.4 |
| B_Mesenchymal Stem Cells | | | |
| 94723_Donor 2 U - | 4.8 | 73139_Uterus_Uterine smooth | 2.1 |
| C_Mesenchymal Stem Cells | | muscle cells | |

TABLE ASF
general oncology screening panel_v_2.4

| | Rel. Exp. (%) | | Rel. Exp. (%) |
|---|---|---|---|
| | Ag3924, | | Ag3924, |
| | Run | | Run |
| Tissue Name | 268143856 | Tissue Nme | 268143856 |
| Colon cancer 1 | 60.3 | Bladder NAT 2 | 3.3 |
| Colon NAT 1 | 29.7 | Bladder NAT 3 | 2.4 |
| Colon cancer 2 | 26.1 | Bladder NAT 4 | 25.7 |
| Colon NAT 2 | 60.7 | Prostate adenocarcinoma 1 | 100.0 |
| Colon cancer 3 | 88.9 | Prostate adenocarcinoma 2 | 14.6 |
| Colon NAT 3 | 88.9 | Prostate adenocarcinoma 3 | 86.5 |
| Colon malignant cancer 4 | 98.6 | Prostate adenocarcinoma 4 | 34.9 |
| Colon NAT 4 | 29.5 | Prostate NAT 5 | 26.2 |
| Lung cancer 1 | 17.3 | Prostate adenocarcinoma 6 | 24.5 |
| Lung NAT 1 | 7.9 | Prostate adenocarcinoma 7 | 39.5 |
| Lung cancer 2 | 31.9 | Prostate adenocarcinoma 8 | 15.2 |
| Lung NAT 2 | 14.8 | Prostate adenocarcinoma 9 | 53.6 |
| Squamous cell carcinoma 3 | 34.2 | Prostate NAT 10 | 12.6 |
| Lung NAT 3 | 5.0 | Kidney cancer 1 | 12.0 |
| Metastatic melanoma 1 | 28.3 | Kidney NAT 1 | 25.9 |
| Melanoma 2 | 4.8 | Kidney cancer 2 | 53.6 |
| Melanoma 3 | 12.9 | Kidney NAT 2 | 64.6 |
| Metastatic melanoma 4 | 42.6 | Kidney cancer 3 | 12.5 |
| Metastatic melanoma 5 | 70.7 | Kidney NAT 3 | 26.6 |
| Bladder cancer 1 | 9.3 | Kidney cancer 4 | 15.0 |
| Bladder NAT 1 | 0.0 | Kidney NAT 4 | 14.6 |
| Bladder cancer 2 | 17.7 | | |

CNS_neurodegeneration_v1.0 Summary: Ag3924 This panel does not show differential expression of this gene in Alzheimer's disease. However, this profile confirms the expression of this gene at moderate levels in the brain. Please see Panel 1.4 for discussion of this gene in the central nervous system.

General_screen_panel_v1.4 Summary: Ag3924 Highest expression of this gene is seen in a breast cancer cell line (CT=25.3). This gene is ubiquitously expressed in this panel, with high to moderate expression seen in brain, colon, gastric, lung, breast, ovarian, and melanoma cancer cell lines. This expression profile suggests a role for this gene product in cell survival and proliferation. Modulation of this gene product may be useful in the treatment of cancer.

Among tissues with metabolic function, this gene is expressed at moderate to high levels in pituitary, adipose, adrenal gland, pancreas, thyroid, and adult and fetalskeletal muscle, heart, and liver. This widespread expression among these tissues suggests that this gene product may play a role in normal neuroendocrine and metabolic function and that disregulated expression of this gene may contribute to neuroendocrine disorders or metabolic diseases, such as obesity and diabetes. This gene encodes a novel glycerol 3-phosphate dehydrogenase (G3PD).

Similar to known cytosolic glycerol 3-phosphate dehydrogenase, this putative G3PD may contribute to glycerol synthesis and link glycolysis with TG production. This gene is highly expressed in skeletal muscle and diabetic skeletal muscle on Panel 5I. Diabetic skeletal muscle has increased glycolytic activity and increased lipid content that interfere with insulin sensitivity. Inhibition of G3PD may balance disproportionate glycolysis and impair accumulation of TG in skeletal muscle. Thus, an antagonist of this novel G3PD may be beneficial for the treatment of diabetes.

This gene is also expressed at high to moderate levels in the CNS, including the hippocampus, thalamus, substantia nigra, amygdala, cerebellum and cerebral cortex. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

In addition, this gene is expressed at much higher levels in fetal lung tissue (CT=27.5) when compared to expression in the adult counterpart (CT=30.5). Thus, expression of this gene may be used to differentiate between the fetal and adult source of this tissue.

Panel 4.1D Summary: Ag3924 Highest expression is seen in a sample derived from an MLR, where the sample was take 7 days after the reaction (CT=27.6). This gene is also expressed at high to moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screen_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Panel 5 Islet Summary: Ag3924 Highest expression is seen in skeletal muscle from a diabetic patient (patient 12) (CT=28). This panel confirms expression of this gene in metabolic tissues including adipose, skeletal muscle and placenta. Please see Panel 1.4 for discussion of this gene in metabolic disease.

General oncology screening panel_v_{—}2.4 Summary: Ag3924 Highest expression is seen in a prostate cancer sample (CT=28.2). Prominent expression is also seen in melanoma samples, as well as in normal and malignant kidney, colon and lung. Thus, modulation of this gene may be useful in the treatment of prostate cancer and melanoma.

AT. CG96613-02 and CG96613-03: Splice Variant of PDK1.

Expression of gene CG96613-02 and CG96613-03 was assessed using the primer-probe sets Ag1778 and Ag5110, described in Tables ATA and ATB. Results of the RTQ-PCR runs are shown in Tables ATC, ATD, ATE, ATF, ATG and ATH. Please note that probe-primer set Agl778 is specific for CG96613-03.

TABLE ATA

Probe Name Ag1778

| | | | Start | SEQ ID |
|---|---|---|---|---|
| Primers | | Length | Position | No |
| Forward | 5′-gattgcccatatcacgtcttta-3′ | 22 | 1241 | 426 |
| Probe | TET-5′-cgcacaatacttccaaggagacctga-3′-TAMRA | 26 | 1263 | 427 |
| Reverse | 5′-gataactgcatctgtcccgtaa-3′ | 22 | 1308 | 428 |

TABLE ATB

Probe Name Ag5110

| | | | Start | SEQ ID |
|---|---|---|---|---|
| Primers | | Length | Position | No |
| Forward | 5′-tgtatggcctgcaagatgat-3′ | 20 | 559 | 429 |
| Probe | TET-5′-tcattcccacaatggcccagg-3′-TAMRA | 21 | 623 | 430 |
| Reverse | 5′-agctctccttgtattcaatcaca-3′ | 23 | 645 | 431 |

TABLE ATC
CNS_neurodegeneration_v1.0

| | Rel. | Rel. | Rel. | | Rel. | Rel. | Rel. |
|---|---|---|---|---|---|---|---|
| | Exp. (%) | Exp. (%) | Exp. (%) | | Exp. (%) | Exp. (%) | Exp. (%) |
| | Ag1778, | Ag5110, | Ag5110, | | Ag1778, | Ag5110, | Ag5110, |
| | Run | Run | Run | | Run | Run | Run |
| Tissue Name | 276596797 | 226442922 | 276596798 | Tissue Name | 276596797 | 226442922 | 276596798 |
| AD 1 Hippo | 11.7 | 6.2 | 5.3 | Control | 6.6 | 12.2 | 17.7 |
| | | | | (Path) 3 | | | |
| | | | | Temporal | | | |
| | | | | Ctx | | | |
| AD 2 Hippo | 31.4 | 7.4 | 20.3 | Control | 33.4 | 15.8 | 13.3 |
| | | | | (Path) 4 | | | |
| | | | | Temporal | | | |
| | | | | Ctx | | | |
| AD 3 Hippo | 12.5 | 5.3 | 4.9 | AD 1 | 23.0 | 7.7 | 8.0 |
| | | | | Occipital Ctx | | | |
| AD 4 Hippo | 5.4 | 9.4 | 0.0 | AD 2 | 0.0 | 0.0 | 0.0 |
| | | | | Occipital Ctx | | | |
| | | | | (Missing) | | | |
| AD 5 Hippo | 82.4 | 79.0 | 45.4 | AD 3 | 12.2 | 6.2 | 5.8 |
| | | | | Occipital Ctx | | | |
| AD 6 Hippo | 54.3 | 88.3 | 70.2 | AD 4 | 16.3 | 18.0 | 7.0 |
| | | | | Occipital Ctx | | | |
| Control 2 | 17.9 | 18.8 | 19.5 | AD 5 | 77.9 | 29.9 | 26.2 |
| Hippo | | | | Occipital Ctx | | | |
| Control 4 | 13.0 | 19.3 | 13.3 | AD 6 | 36.9 | 18.9 | 18.8 |
| Hippo | | | | Occipital Ctx | | | |
| Control | 11.0 | 7.5 | 16.3 | Control 1 | 6.2 | 6.8 | 5.4 |
| (Path) 3 | | | | Occipital Ctx | | | |
| Hippo | | | | | | | |
| AD 1 | 20.3 | 14.6 | 11.0 | Control 2 | 54.0 | 44.8 | 51.4 |
| Temporal | | | | Occipital Ctx | | | |
| Ctx | | | | | | | |
| AD2 | 29.9 | 16.6 | 21.8 | Control 3 | 32.3 | 4.9 | 26.8 |
| Temporal | | | | Occipital Ctx | | | |
| Ctx | | | | | | | |
| AD 3 | 11.7 | 8.4 | 17.7 | Control 4 | 7.5 | 9.2 | 5.3 |
| Temporal | | | | Occipital Ctx | | | |
| Ctx | | | | | | | |
| AD 4 | 20.2 | 5.6 | 19.6 | Control | 60.3 | 24.7 | 41.8 |
| Temporal | | | | (Path) 1 | | | |
| Ctx | | | | Occipital Ctx | | | |
| AD 5 Inf | 72.2 | 47.0 | 46.3 | Control | 12.8 | 9.2 | 6.3 |
| Temporal | | | | (Path) 2 | | | |
| Ctx | | | | Occipital Ctx | | | |
| AD 5 Sup | 39.5 | 51.1 | 44.1 | Control | 5.5 | 0.9 | 0.0 |
| Temporal | | | | (Path) 3 | | | |
| Ctx | | | | Occipital Ctx | | | |
| AD 6 Inf | 75.3 | 84.1 | 84.1 | Control | 16.6 | 15.5 | 12.3 |
| Temporal | | | | (Path) 4 | | | |
| Ctx | | | | Occipital Ctx | | | |
| AD 6 Sup | 100.0 | 100.0 | 100.0 | Control 1 | 10.0 | 10.0 | 3.6 |
| Temporal | | | | Parietal Ctx | | | |
| Ctx | | | | | | | |
| Control 1 | 11.2 | 10.4 | 3.9 | Control 2 | 46.0 | 57.0 | 27.5 |
| Temporal | | | | Parietal Ctx | | | |
| Ctx | | | | | | | |
| Control 2 | 25.3 | 21.6 | 36.3 | Control 3 | 23.5 | 18.3 | 16.6 |
| Temporal | | | | Parietal Ctx | | | |
| Ctx | | | | | | | |
| Control 3 | 31.2 | 37.9 | 38.2 | Control | 78.5 | 39.2 | 52.5 |
| Temporal Ctx | | | | (Path) 1 | | | |
| Ctx | | | | Parietal Ctx | | | |
| Control 3 | 11.7 | 8.4 | 8.8 | Control | 23.5 | 12.5 | 14.9 |
| Temporal | | | | (Path) 2 | | | |
| Ctx | | | | Parietal Ctx | | | |
| Control | 36.6 | 53.6 | 46.7 | Control | 9.5 | 13.9 | 5.8 |
| (Path) 1 | | | | (Path) 3 | | | |
| Temporal | | | | Parietal Ctx | | | |
| Ctx | | | | | | | |
| Control | 46.0 | 29.7 | 32.5 | Control | 46.0 | 58.6 | 39.2 |
| (Path) 2 | | | | (Path) 4 | | | |
| Temporal | | | | Parietal Ctx | | | |
| Ctx | | | | | | | |

TABLE ATD
General_screening_panel_v1.5

| | Rel. | | Rel. |
|---|---|---|---|
| | Exp. (%) | | Exp. (%) |
| | Ag5110, | | Ag5110, |
| | Run | | Run |
| Tissue Name | 228980585 | issue Name | 228980585 |
| Adipose | 5.4 | Renal ca. TK-10 | 11.7 |
| Melanoma* Hs688(A).T | 10.7 | Bladder | 12.2 |
| Melanoma* Hs688(B).T | 5.8 | Gastric ca. (liver met.) NCI-N87 | 3.8 |
| Melanoma* M14 | 19.5 | Gastric ca. KATO III | 10.6 |
| Melanoma* LOXIMVI | 17.3 | Colon ca. SW-948 | 2.6 |
| Melanoma* SK-MEL-5 | 29.9 | Colon ca. SW480 | 16.6 |
| Squamous cell carcinoma SCC-4 | 4.2 | Colon ca.* (SW480 met) SW620 | 10.8 |
| Testis Pool | 9.2 | Colon ca. HT29 | 17.0 |
| Prostate ca.* (bone met) PC-3 | 48.0 | Colon ca. HCT-116 | 6.7 |
| Prostate Pool | 0.6 | Colon ca. CaCo-2 | 9.8 |
| Placenta | 0.5 | Colon cancer tissue | 7.1 |
| Uterus Pool | 2.3 | Colon ca. SW1116 | 2.5 |
| Ovarian ca. OVCAR-3 | 5.5 | Colon ca. Colo-205 | 3.5 |
| Ovarian ca. SK-OV-3 | 11.8 | Colon ca. SW-48 | 4.7 |
| Ovarian ca. OVCAR-4 | 7.9 | Colon Pool | 0.8 |
| Ovarian ca. OVCAR-5 | 17.4 | Small Intestine Pool | 1.2 |
| Ovarian ca. IGROV-1 | 8.7 | Stomach Pool | 2.2 |
| Ovarian ca. OVCAR-8 | 8.2 | Bone Marrow Pool | 1.2 |
| Ovary | 0.3 | Fetal Heart | 13.0 |
| Breast ca. MCF-7 | 4.3 | Heart Pool | 4.0 |
| Breast ca. MDA-MB-231 | 25.0 | Lymph Node Pool | 0.9 |
| Breast ca. BT 549 | 21.3 | Fetal Skeletal Muscle | 0.6 |
| Breast ca. T47D | 2.7 | Skeletal Muscle Pool | 1.7 |
| Breast ca. MDA-N | 17.2 | Spleen Pool | 7.5 |
| Breast Pool | 0.7 | Thymus Pool | 11.6 |
| Trachea | 21.9 | CNS cancer (glio/astro) U87-MG | 48.3 |
| Lung | 1.2 | CNS cancer (glio/astro) U-118-MG | 71.7 |
| Fetal Lung | 4.0 | CNS cancer (neuro; met) SK-N-AS | 7.2 |
| Lung ca. NCI-N417 | 11.3 | CNS cancer (astro) SF-539 | 16.6 |
| Lung ca. LX-1 | 20.3 | CNS cancer (astro) SNB-75 | 24.7 |
| Lung ca. NCI-H146 | 5.5 | CNS cancer (glio) SNB-19 | 11.0 |
| Lung ca. SHP-77 | 17.7 | CNS cancer (glio) SF-295 | 27.5 |
| Lung ca. A549 | 6.9 | Brain (Amygdala) Pool | 2.0 |
| Lung ca. NCI-H526 | 11.9 | Brain (cerebellum) | 5.2 |
| Lung ca. NCI-H23 | 4.7 | Brain (fetal) | 1.0 |
| Lung ca. NCI-H460 | 32.3 | Brain (Hippocampus) Pool | 2.0 |
| Lung ca. HOP-62 | 9.7 | Cerebral Cortex Pool | 1.9 |
| Lung ca. NCI-H522 | 12.8 | Brain (Substantia nigra) Pool | 1.6 |
| Liver | 0.4 | Brain (Thalamus) Pool | 1.7 |
| Fetal Liver | 100.0 | Brain (whole) | 3.0 |
| Liver ca. HepG2 | 15.4 | Spinal Cord Pool | 1.0 |
| Kidney Pool | 1.6 | Adrenal Gland | 14.9 |
| Fetal Kidney | 2.2 | Pituitary gland Pool | 0.4 |
| Renal ca. 786-0 | 10.5 | Salivary Gland | 6.1 |
| Renal ca. A498 | 0.2 | Thyroid (female) | 0.5 |
| Renal ca. ACHN | 8.4 | Pancreatic ca. CAPAN2 | 2.6 |
| Renal ca. UO-31 | 3.7 | Pancreas Pool | 1.3 |

TABLE ATE
General_screening_panel v1.6

| | Rel. | Rel. | | Rel. | Rel. |
|---|---|---|---|---|---|
| | Exp. (%) | Exp. (%) | | Exp. (%) | Exp. (%) |
| | Ag1778, | Ag5110, | | Ag1778, | Ag5110, |
| | Run | Run | | Run | Run |
| Tissue Name | 277218713 | 277218715 | Tissue Name | 277218713 | 277218715 |
| Adipose | 8.8 | 8.7 | Renal ca. TK-10 | 31.6 | 13.5 |
| Melanoma* | 45.1 | 15.5 | Bladder | 23.3 | 14.5 |
| Hs688(A).T | | | | | |
| Melanoma* | 34.6 | 11.7 | Gastric ca. (liver | 22.1 | 5.0 |
| Hs688(B).T | | | met.) NCI-N87 | | |
| Melanoma* M14 | 29.3 | 11.6 | Gastric ca. KATO | 9.0 | 15.3 |
| | | | III | | |
| Melanoma* | 16.6 | 32.1 | Colon ca. SW-948 | 9.2 | 4.4 |
| LOXIMVI | | | | | |
| Melanoma* | 23.0 | 36.9 | Colon ca. SW480 | 35.8 | 22.5 |
| SK-MEL-5 | | | | | |
| Squamous Cell | 16.6 | 7.2 | Colon ca.* (SW480 | 24.0 | 11.9 |
| carcinoma SCC-4 | | | met) SW620 | | |
| Testis Pool | 8.9 | 8.5 | Colon ca. HT29 | 32.1 | 21.5 |
| Prostate ca.* (bone | 100.0 | 50.7 | Colon ca. HCT-116 | 17.9 | 9.3 |
| met) PC-3 | | | | | |
| Prostate Pool | 5.7 | 1.7 | Colon ca. CaCo-2 | 21.6 | 13.5 |
| Placenta | 1.6 | 0.3 | Colon cancer tissue | 3.2 | 10.5 |
| Uterus Pool | 3.5 | 3.1 | Colon ca. SW1116 | 3.8 | 2.7 |
| Ovarian ca. | 11.6 | 9.5 | Colon ca. Colo-205 | 6.7 | 4.5 |
| OVCAR-3 | | | | | |
| Ovarian ca. SK-OV-3 | 33.0 | 20.3 | Colon ca. SW-48 | 12.1 | 5.2 |
| Ovarian ca. | 11.4 | 10.7 | Colon Pool | 6.6 | 1.8 |
| OVCAR-4 | | | | | |
| Ovarian ca. | 28.1 | 24.8 | Small Intestine Pool | 9.0 | 3.0 |
| OVCAR-5 | | | | | |
| Ovarian ca. | 29.1 | 12.7 | Stomach Pool | 5.6 | 4.5 |
| IGROV-1 | | | | | |
| Ovarian ca. | 15.9 | 0.1 | Bone Marrow Pool | 5.1 | 2.4 |
| OVCAR-8 | | | | | |
| Ovary | 4.4 | 1.6 | Fetal Heart | 61.6 | 26.4 |
| Breast ca. MCF-7 | 5.9 | 3.6 | Heart Pool | 6.8 | 8.8 |
| Breast ca. | 79.0 | 34.4 | Lymph Node Pool | 10.4 | 0.8 |
| MDA-MB-231 | | | | | |
| Breast ca. BT 549 | 35.6 | 15.9 | Fetal Skeletal | 6.6 | 0.6 |
| | | | Muscle | | |
| Breast ca. T47D | 3.0 | 3.4 | Skeletal Muscle | 0.9 | 0.7 |
| | | | Pool | | |
| Breast ca. MDA-N | 20.7 | 20.9 | Spleen Pool | 19.2 | 13.0 |
| Breast Pool | 7.4 | 1.9 | Thymus Pool | 20.2 | 12.6 |
| Trachea | 23.8 | 33.7 | CNS cancer | 47.0 | 51.1 |
| | | | (glio/astro) | | |
| | | | U87-MG | | |
| Lung | 4.6 | 1.0 | CNS cancer | 43.2 | 100.0 |
| | | | (glio/astro) | | |
| | | | U-118-MG | | |
| Fetal Lung | 17.4 | 8.1 | CNS cancer | 14.1 | 7.7 |
| | | | (neuro; met) | | |
| | | | SK-N-AS | | |
| Lung ca. NCI-N417 | 16.2 | 16.0 | CNS cancer (astro) | 35.1 | 28.3 |
| | | | SF-539 | | |
| Lung ca. LX-1 | 38.7 | 8.8 | CNS cancer (astro) | 50.3 | 30.8 |
| | | | SNB-75 | | |
| Lung ca. NCI-H146 | 16.7 | 5.9 | CNS cancer (glio) | 34.4 | 13.1 |
| | | | SNB-19 | | |
| Lung ca. SHP-77 | 53.2 | 25.9 | CNS cancer (glio) | 93.3 | 46.0 |
| | | | SF-295 | | |
| Lung ca. A549 | 10.9 | 9.9 | Brain (Amygdala) | 7.7 | 2.3 |
| | | | Pool | | |
| Lung ca. NCI-H526 | 10.1 | 10.9 | Brain (cerebellum) | 24.7 | 5.3 |
| Lung ca. NCI-H23 | 12.2 | 9.2 | Brain (fetal) | 9.7 | 1.3 |
| Lung ca. NCI-H460 | 57.4 | 57.8 | Brain | 9.7 | 2.8 |
| | | | (Hippocampus) | | |
| | | | Pool | | |
| Lung ca. HOP-62 | 39.0 | 9.7 | Cerebral Cortex Pool | 9.6 | 3.3 |
| Lung ca. NCI-H522 | 19.5 | 13.3 | Brain (Substantia | 6.0 | 2.8 |
| | | | nigra) Pool | | |
| Liver | 1.5 | 0.6 | Brain (Thalamus) | 15.3 | 1.9 |
| | | | Pool | | |
| Fetal Liver | 15.1 | 6.0 | Brain (whole) | 9.5 | 3.3 |
| Liver ca. HepG2 | 41.5 | 18.2 | Spinal Cord Pool | 5.8 | 2.1 |
| Kidney Pool | 9.6 | 2.0 | Adrenal Gland | 27.5 | 23.3 |
| Fetal Kidney | 14.7 | 2.6 | Pituitary gland Pool | 2.5 | 1.0 |
| Renal ca. 786-0 | 14.5 | 11.0 | Salivary Gland | 9.8 | 10.4 |
| Renal ca. A498 | 2.2 | 0.9 | Thyroid (female) | 1.5 | 1.9 |
| Renal ca. ACHN | 9.5 | 10.8 | Pancreatic ca. | 9.7 | 5.3 |
| | | | CAPAN2 | | |
| Renal ca. UO-31 | 13.4 | 4.6 | Pancreas Pool | 18.0 | 7.2 |

TABLE ATF
Panel 1.3D

| | Rel. | | Rel. |
|---|---|---|---|
| | Exp. (% | | Exp. (%) |
| | Ag1778, | | Ag1778, |
| | Run | | Run |
| Tissue Name | 157790405 | Tissue Name | 157790405 |
| Liver adenocarcinoma | 6.7 | Kidney (fetal) | 12.1 |
| Pancreas | 1.3 | Renal ca. 786-0 | 6.8 |
| Pancreatic ca. CAPAN 2 | 2.1 | Renal ca. A498 | 12.2 |
| Adrenal gland | 18.7 | Renal ca. RXF 393 | 15.0 |
| Thyroid | 2.9 | Renal ca. ACHN | 3.2 |
| Salivary gland | 6.2 | Renal ca. UO-31 | 8.4 |
| Pituitary gland | 5.7 | Renal ca. TK-10 | 3.6 |
| Brain (fetal) | 2.5 | Liver | 3.0 |
| Brain (whole) | 4.8 | Liver (fetal) | 14.7 |
| Brain (amygdala) | 6.3 | Liver ca. (hepatoblast) HepG2 | 25.5 |
| Brain (cerebellum) | 5.4 | Lung | 13.7 |
| Brain (hippocampus) | 22.8 | Lung (fetal) | 5.3 |
| Brain (substantia nigra) | 1.1 | Lung ca. (small cell) LX-1 | 14.5 |
| Brain (thalamus) | 3.3 | Lung ca. (small cell) NCI-H69 | 4.9 |
| Cerebral Cortex | 14.7 | Lung ca. (s.cell var.) SHP-77 | 36.1 |
| Spinal cord | 2.3 | Lung ca. (large cell)NCI-H460 | 12.9 |
| glio/astro U87-MG | 21.6 | Lung ca. (non-sm. cell) A549 | 8.1 |
| glio/astro U-118-MG | 56.3 | Lung ca. (non-s.cell) NCI-H23 | 7.3 |
| astrocytoma SW1783 | 31.2 | Lung ca. (non-s.cell) HOP-62 | 12.8 |
| neuro*; met SK-N-AS | 30.4 | Lung ca. (non-s.cl) NCI-H522 | 4.5 |
| astrocytoma SF-539 | 22.2 | Lung ca. (squam.) SW 900 | 1.5 |
| astrocytoma SNB-75 | 12.6 | Lung ca. (squam.) NCI-H596 | 0.7 |
| glioma SNB-19 | 29.9 | Mammary gland | 9.7 |
| glioma U251 | 22.2 | Breast ca.* (pl.ef) MCF-7 | 4.6 |
| glioma SF-295 | 20.3 | Breast ca.* (pl.ef) MDA-MB-231 | 100.0 |
| Heart (fetal) | 35.4 | Breast ca.* (pl.ef) T47D | 5.1 |
| Heart | 4.5 | Breast ca. BT-549 | 45.1 |
| Skeletal muscle (fetal) | 26.1 | Breast ca. MDA-N | 28.9 |
| Skeletal muscle | 3.1 | Ovary | 4.0 |
| Bone marrow | 13.1 | Ovarian ca. OVCAR-3 | 4.5 |
| Thymus | 6.2 | Ovarian ca. OVCAR-4 | 3.5 |
| Spleen | 15.5 | Ovarian ca. OVCAR-5 | 13.4 |
| Lymph node | 16.3 | Ovarian ca. OVCAR-8 | 3.1 |
| Colorectal | 7.9 | Ovarian ca. IGROV-1 | 4.2 |
| Stomach | 14.5 | Ovarian ca.* (ascites) SK-OV-3 | 13.2 |
| Small intestine | 15.5 | Uterus | 3.1 |
| Colon ca. SW480 | 9.7 | Placenta | 4.3 |
| Colon ca.* SW620(SW480 met) | 10.7 | Prostate | 2.2 |
| Colon ca. HT29 | 25.5 | Prostate ca.* (bone met)PC-3 | 16.7 |
| Colon ca. HCT-116 | 5.1 | Testis | 20.2 |
| Colon ca. CaCo-2 | 8.1 | Melanoma Hs688(A).T | 7.1 |
| Colon ca. tissue(ODO3866) | 8.4 | Melanoma* (met) Hs688(B).T | 3.8 |
| Colon ca. HCC-2998 | 12.2 | Melanoma UACC-62 | 2.0 |
| Gastric ca.* (liver met) NCI-N87 | 11.1 | Melanoma M14 | 11.4 |
| Bladder | 8.0 | Melanoma LOX IMVI | 10.8 |
| Trachea | 17.7 | Melanoma* (met) SK-MEL-5 | 5.2 |
| Kidney | 0.7 | Adipose | 4.9 |

TABLE ATG
Panel 4.1D

| | Rel. | Rel. | Rel. | Rel. | Rel. |
|---|---|---|---|---|---|
| | Exp. (%) | Exp. (%) | Exp. (%) | Exp. (%) | Exp. (%) |
| | Ag1778, | Ag1778, | Ag5110, | Ag5110, | Ag5110, |
| | Run | Run | Run | Run | Run |
| Tissue Name | 276596860 | 276686878 | 226444095 | 276596862 | 276686880 |
| Secondary Th1 act | 23.5 | 26.8 | 13.9 | 14.9 | 9.0 |
| Secondary Th2 act | 28.7 | 28.1 | 11.4 | 14.8 | 17.9 |
| Secondary Tr1 act | 5.4 | 8.4 | 7.9 | 1.9 | 4.5 |
| Secondary Th1 rest | 2.9 | 3.8 | 6.3 | 1.0 | 1.5 |
| Secondary Th2 rest | 7.4 | 4.3 | 11.3 | 4.3 | 2.7 |
| Secondary Tr1 rest | 4.3 | 4.9 | 6.6 | 4.8 | 1.4 |
| Primary Th1 act | 4.5 | 5.6 | 13.9 | 5.0 | 1.8 |
| Primary Th2 act | 23.2 | 16.8 | 14.4 | 14.4 | 16.5 |
| Primary Tr1 act | 22.2 | 23.3 | 13.9 | 11.1 | 12.3 |
| Primary Th1 rest | 3.1 | 3.3 | 2.2 | 0.0 | 0.0 |
| Primary Th2 rest | 6.8 | 4.2 | 5.6 | 0.0 | 0.0 |
| Primary Tr1 rest | 2.6 | 3.6 | 10.3 | 0.7 | 0.0 |
| CD45RA CD4 | 25.5 | 26.4 | 9.5 | 18.3 | 16.2 |
| lymphocyte act | | | | | |
| CD45RO CD4 | 40.1 | 27.2 | 22.1 | 27.9 | 22.4 |
| lymphocyte act | | | | | |
| CD8 lymphocyte act | 5.1 | 7.4 | 13.1 | 8.1 | 2.4 |
| Secondary CD8 | 3.3 | 5.1 | 20.9 | 32.3 | 5.1 |
| lymphocyte rest | | | | | |
| Secondary CD8 | 4.3 | 3.7 | 3.3 | 1.3 | 0.0 |
| lymphocyte act | | | | | |
| CD4 lymphocyte none | 13.3 | 8.6 | 13.7 | 4 3 | 4.9 |
| 2ry | 3.2 | 5.2 | 8.1 | 3.1 | 2.4 |
| Th1/Th2/Tr1_anti-CD95 | | | | | |
| CH11 | | | | | |
| LAK cells rest | 13.2 | 6.7 | 10.1 | 5.6 | 4.6 |
| LAK cells IL-2 | 9.1 | 8.0 | 11.1 | 6.2 | 3.5 |
| LAK cells IL-2 + IL-12 | 0.8 | 1.3 | 11.0 | 1.7 | 0.0 |
| LAK cells IL-2 + IFN | 9.2 | 8.5 | 12.2 | 4.8 | 7.6 |
| gamma | | | | | |
| LAK cells IL-2 + IL-18 | 6.4 | 5.1 | 15.6 | 3.7 | 12.2 |
| LAK cells | 100.0 | 100.0 | 100.0 | 100.0 | 100.0 |
| PMA/ionomycin | | | | | |
| NK Cells IL-2 rest | 27.5 | 17.8 | 8.7 | 7.1 | 14.7 |
| Two Way MLR 3 day | 16.8 | 21.2 | 16.3 | 5.1 | 10.7 |
| Two Way MLR 5 day | 2.9 | 2.7 | 4.2 | 1.7 | 0.0 |
| Two Way MLR 7 day | 6.2 | 2.6 | 3.4 | 1.9 | 2.6 |
| PBMC rest | 3.6 | 3.7 | 5.9 | 2.3 | 3.2 |
| PBMC PWM | 9.5 | 6.9 | 4.5 | 1.7 | 1.6 |
| PBMC PHA-L | 6.9 | 8.0 | 8.7 | 5.0 | 3.4 |
| Ramos (B cell) none | 7.7 | 4.2 | 4.7 | 0.6 | 1.4 |
| Ramos (B cell) ionomycin | 36.6 | 32.1 | 11.9 | 9.2 | 6.0 |
| B lymphocytes PWM | 11.7 | 4.9 | 6.7 | 4.4 | 4.3 |
| B lymphocytes CD40L | 34.2 | 21.0 | 13.2 | 15.2 | 19.8 |
| and IL-4 | | | | | |
| EOL-1 dbcAMP | 52.1 | 34.4 | 11.0 | 10.8 | 15.6 |
| EOL- 1 dbcAMP | 9.8 | 6.0 | 3.5 | 1.4 | 5.8 |
| PMA/ionomycin | | | | | |
| Dendritic cells none | 9.5 | 7.7 | 7.3 | 6.3 | 5.4 |
| Dendritic cells LPS | 5.6 | 5.0 | 6.6 | 1.1 | 2.0 |
| Dendritic cells anti-CD40 | 3.6 | 4.2 | 7.0 | 1.3 | 1.5 |
| Monocytes rest | 4.9 | 3.1 | 6.9 | 1.2 | 0.0 |
| Monocytes LPS | 11.3 | 8.4 | 6.8 | 2.9 | 0.0 |
| Macrophages rest | 5.7 | 10.2 | 5.7 | 1.9 | 0.0 |
| Macrophages LPS | 3.2 | 3.0 | 5.2 | 0.7 | 3.6 |
| HUVEC none | 6.0 | 4.2 | 1.8 | 1.3 | 5.2 |
| HUVEC starved | 11.0 | 9.5 | 4.4 | 5.9 | 2.3 |
| HUVEC IL-1beta | 11.9 | 10.1 | 4.9 | 8.1 | 9.0 |
| HUVEC IFN gamma | 9.2 | 9.4 | 5.5 | 2.7 | 6.5 |
| HUVEC TNF alpha + IFN | 3.8 | 3.6 | 4.1 | 3.5 | 1.8 |
| gamma | | | | | |
| HUVEC TNF alpha + IL4 | 2.7 | 2.8 | 5.5 | 0.0 | 0.0 |
| HUVEC IL-11 | 4.3 | 5.3 | 3.5 | 3.4 | 0.0 |
| Lung Microvascular EC | 25.3 | 23.3 | 7.5 | 6.9 | 6.2 |
| none | | | | | |
| Lung Microvascular EC | 9.2 | 7.0 | 7.9 | 2.6 | 2.2 |
| TNF alpha + IL-1beta | | | | | |
| Microvascular Dermal EC | 1.8 | 2.1 | 3.8 | 0.0 | 0.0 |
| none | | | | | |
| Microvasular Dermal EC | 2.0 | 2.6 | 1.9 | 1.3 | 0.0 |
| TNF alpha + IL-1beta | | | | | |
| Bronchial epithelium | 8.8 | 14.0 | 10.6 | 3.3 | 3.3 |
| TNF alpha + IL1beta | | | | | |
| Small airway epithelium | 10.7 | 3.0 | 2.4 | 3.4 | 6.0 |
| none | | | | | |
| Small airway epithelium | 31.9 | 31.0 | 21.9 | 30.4 | 15.8 |
| TNF alpha + IL-1beta | | | | | |
| Coronery artery SMC rest | 25.2 | 19.6 | 9.1 | 13.3 | 13.4 |
| Coronery artery SMC | 27.5 | 19.6 | 5.5 | 7.8 | 15.2 |
| TNF alpha + IL-1beta | | | | | |
| Astrocytes rest | 8.2 | 15.3 | 2.4 | 1.9 | 2.8 |
| Astrocytes TNF alpha + | 5.2 | 2.7 | 3.4 | 0.0 | 5.3 |
| IL-1beta | | | | | |
| KU-812 (Basophil) rest | 10.7 | 8.1 | 3.5 | 2.0 | 0.0 |
| KU-812 (Basophil) | 37.1 | 25.5 | 11.6 | 8.9 | 5.2 |
| PMA/ionomycin | | | | | |
| CCD1106 (Keratinocytes) | 20.6 | 20.9 | 13.2 | 4.5 | 6.9 |
| none | | | | | |
| CCD11O6 (Keratinocytes) | 14.1 | 22.7 | 17.8 | 7.7 | 2.3 |
| TNF alpha + IL-1beta | | | | | |
| Liver cirrhosis | 11.4 | 8.5 | 7.4 | 1.4 | 1.4 |
| NCI-H292 none | 12.9 | 7.6 | 7.1 | 5.5 | 7.5 |
| NCI-H292 IL-4 | 11.9 | 12.2 | 4.3 | 4.8 | 5.8 |
| NCI-H292 IL-9 | 16.8 | 12.7 | 7.0 | 3.7 | 11.4 |
| NCI-H292 IL-13 | 12.5 | 10.0 | 6.5 | 4.2 | 7.3 |
| NCI-H292 IFN gamma | 3.9 | 4.1 | 7.6 | 2.6 | 4.2 |
| HPAEC none | 1.7 | 2.9 | 2.6 | 0.0 | 0.0 |
| HPAEC TNF alpha + IL-1 | 10.6 | 7.2 | 2.9 | 2.7 | 3.3 |
| beta | | | | | |
| Lung fibroblast none | 31.2 | 24.1 | 4.5 | 8.7 | 5.8 |
| Lung fibroblast TNF | 24.3 | 21.6 | 6.6 | 7.5 | 11.2 |
| alpha + IL-1beta | | | | | |
| Lung fibroblast IL-4 | 6.5 | 1.1 | 1.8 | 3.2 | 4.0 |
| Lung fibroblast IL-9 | 19.2 | 28.3 | 8.2 | 6.7 | 7.7 |
| Lung fibroblast IL-13 | 8.2 | 5.1 | 2.9 | 0.0 | 3.6 |
| Lung fibroblast IFN | 15.3 | 14.9 | 5.5 | 3.8 | 12.9 |
| gamma | | | | | |
| Dermal fibroblast | 25.0 | 23.3 | 7.8 | 4.6 | 11.0 |
| CCD1070 rest | | | | | |
| Dermal fibroblast | 74.2 | 45.1 | 14.1 | 23.2 | 36.3 |
| CCD1070 TNF alpha | | | | | |
| Dermal fibroblast | 23.3 | 22.4 | 4.3 | 3.9 | 5.7 |
| CCD1070 IL-1beta | | | | | |
| Dermal fibroblast IFN | 3.4 | 3.9 | 2.0 | 0.9 | 0.0 |
| gamma | | | | | |
| Dermal fibroblast IL-4 | 6.8 | 8.2 | 3.3 | 2.6 | 3.0 |
| Dermal Fibroblasts rest | 11.2 | 7.8 | 2.8 | 2.7 | 3.8 |
| Neutrophils TNFa + LPS | 4.5 | 1.6 | 1.6 | 1.8 | 0.0 |
| Neutrophils rest | 28.9 | 31.2 | 12.1 | 15.9 | 20.2 |
| Colon | 2.3 | 1.5 | 2.3 | 0.0 | 2.3 |
| Lung | 2.0 | 2.4 | 3.7 | 0.9 | 1.6 |
| Thymus | 13.0 | 14.6 | 6.6 | 0.0 | 5.1 |
| Kidney | 7.9 | 7.5 | 1.7 | 1.1 | 2.8 |

TABLE ATH
general oncology screening panel_v_2.4

| | Rel. Exp. (%) | | Rel. Exp. (%) |
|---|---|---|---|
| | Ag5110, | | Ag5110, |
| | Run | | Run |
| Tissue Name | 259939210 | Tissue Nme | 259939210 |
| Colon cancer 1 | 6.5 | Bladder NAT 2 | 0.0 |
| Colon NAT 1 | 5.9 | Bladder NAT 3 | 0.0 |
| Colon cancer 2 | 6.0 | Bladder NAT 4 | 0.0 |
| Colon NAT 2 | 14.2 | Prostate adenocarcinoma 1 | 1.2 |
| Colon cancer 3 | 23.7 | Prostate adenocarcinoma 2 | 0.0 |
| Colon NAT 3 | 15.7 | Prostate adenocarcinoma 3 | 1.6 |
| Colon malignant cancer 4 | 41.5 | Prostate adenocarcinoma 4 | 14.2 |
| Colon NAT 4 | 4.2 | Prostate NAT 5 | 0.9 |
| Lung cancer 1 | 7.5 | Prostate adenocarcinoma 6 | 0.0 |
| Lung NAT 1 | 0.0 | Prostate adenocarcinoma 7 | 0.7 |
| Lung cancer 2 | 28.5 | Prostate adenocarcinoma 8 | 0.0 |
| Lung NAT 2 | 1.2 | Prostate adenocarcinoma 9 | 3.0 |
| Squamous cell carcinoma 3 | 42.3 | Prostate NAT 10 | 0.0 |
| Lung NAT 3 | 0.0 | Kidney cancer 1 | 34.2 |
| Metastatic melanoma 1 | 1.4 | Kidney NAT 1 | 4.5 |
| Melanoma 2 | 10.4 | Kidney cancer 2 | 100.0 |
| Melanoma 3 | 2.1 | Kidney NAT 2 | 3.2 |
| Metastatic melanoma 4 | 2.2 | Kidney cancer 3 | 19.6 |
| Metastatic melanoma 5 | 4.5 | Kidney NAT 3 | 1.1 |
| Bladder cancer 1 | 0.0 | Kidney cancer 4 | 37.1 |
| Bladder NAT 1 | 0.0 | Kidney NAT 4 | 1.0 |
| Bladder cancer 2 | 2.3 | | |

CNS_neurodegeneration_v1.0 Summary: Ag1778/Ag5110 This panel confirms the expression of this gene at low levels in the brains of an independent group of individuals. However, no differential expression of this gene was detected between Alzheimer's diseased postmortem brains and those of non-demented controls in this experiment. Please see Panel 1.5 for a discussion of this gene in treatment of central nervous system disorders.

General_screen_panel_v1.5 Summary: Ag5110 Highest expression of this gene is detected in fetal liver (CT=29.4). Interestingly, this gene is expressed at much higher levels in fetal when compared to adult liver (CT=37). This observation suggests that expression of this gene can be used to distinguish fetal from adult liver. In addition, the relative overexpression of this gene in fetal tissue suggests that the protein product may enhance liver growth or development in the fetus and thus may also act in a regenerative capacity in the adult. Therefore, therapeutic modulation of the protein encoded by this gene could be useful in treatment of liver related diseases.

Among tissues with metabolic or endocrine function, this gene is expressed at low levels in adipose, adrenal gland, heart, fetal liver and stomach. This gene codes for a splice variant of pyruvate dehydrogenase [lipoamide] kinase (PDK). Pyruvate dehydrogenase kinase (PDK) catalyzes phosphorylation and inactivation of the pyruvate dehydrogenase complex (PDC). Inactivation of PDC by increased PDK activity promotes gluconeogenesis by conserving three-carbon substrates. This helps maintain glucose levels during starvation, but is detrimental in diabetes (Huang et al., 2002, Diabetes 51(2):276-83, PMID: 11812733). Therefore, therapeutic modulation of the activity of PKD encoded by gene may be useful in the treatment of endocrine/metabolically related diseases, such as obesity and diabetes.

In addition, this gene is expressed at low levels in cerebellum and whole brain. Therefore, therapeutic modulation of this gene product may be useful in the treatment of neurological disorders such as Alzheimer's disease, Parkinson's disease, epilepsy, multiple sclerosis, schizophrenia and depression.

Moderate to low levels of expression of this gene is also seen in cluster of cancer cell lines derived from pancreatic, gastric, colon, lung, liver, renal, breast, ovarian, prostate, squamous cell carcinoma, melanoma and brain cancers. Thus, expression of this gene could be used as a marker to detect the presence of these cancers. Furthermore, therapeutic modulation of the expression or function of this gene may be effective in the treatment of pancreatic, gastric, colon, lung, liver, renal, breast, ovarian, prostate, squamous cell carcinoma, melanoma and brain cancers.

General_screen_panel_v1.6 Summary: Ag1778/Ag5110 Two experiments with different probe and primer sets are in good agreement. Highest expression of this gene is detected in a prostate cancer PC3 and a brain cancer U-118-MG cell lines (CTs=25-29.8). Expression in this panel correlates with pattern seen in panel 1.5. Moderate to low levels of expression of this gene is detected in tissues with metabolic/endocrine functions such as pancreas, adipose, adrenal gland, heart, fetal liver and gastrointestinal tract, in brain including cerebellum, cerebral cortex, substantia nigra and the whole brain and also in number of cancer cell lines derived from pancreatic, gastric, colon, lung, liver, renal, breast, ovarian, prostate, squamous cell carcinoma, melanoma and brain cancers. Please see panel 1.5 for further discussion on the utility of this gene.

Panel 1.3D Summary: Ag1778 Highest expression of this gene is detected in a breast cancer cell line (CT=27.4). Expression in this panel correlates with pattern seen in panel 1.5. Moderate to low levels of expression of this gene is detected in tissues with metabolic/endocrine functions such as pancreas, adrenal gland, heart, fetal liver and gastrointestinal tract, in brain including cerebellum, cerebral cortex, substantia nigra and the whole brain and also in number of cancer cell lines derived from pancreatic, gastric, colon, lung, liver, renal, breast, ovarian, prostate, squamous cell carcinoma, melanoma and brain cancers. Please see panel 1.5 for further discussion of this gene.

Panel 4.1D Summary: Ag1778/Ag5110 Five experiments with the two different probe-primer sets are in good agreement. Highest expression of this gene is detected in PMA/ionomycin treated LAK cells. These cells are involved in tumor immunology and cell clearance of virally and bacterial infected cells as well as tumors. Therefore, modulation of the function of the protein encoded by this gene through the application of a small molecule drug or antibody may alter the functions of these cells and lead to improvement of symptoms associated with these conditions.

Low levels of expression of this gene is also seen in naive and memory T cells, resting secondary CD8 lymphocytes, cytokine activated small airway epithelium, and resting neutrophils. Therefore, therapeutic modulation of this gene or its protein product may be useful in the treatment of Therefore, therapeutic modulation of this gene product may ameliorate symptoms/conditions associated with autoimmune and inflammatory disorders including psoriasis, allergy, asthma, inflammatory bowel disease, rheumatoid arthritis and osteoarthritis

General oncology screening panel_v_{—}2.4 Summary: Ag5110 Highest expression of this gene is detected in kidney cancer (CT=32). Low levels of expression of this gene is also seen in colon, lung, prostate and kidney cancer. Higher levels of expression of this gene is associated with cancer as compared to corresponding normal tissue. Therefore, expression of this gene may be used as diagnostic marker for the detection of these cancers.

Furthermore, therapeutic modulation of this gene or its protein product may be useful in the treatment of colon, lung, prostate and kidney cancers.

AU. CG96736-01: Neutral amino acid transporter B.

Expression of gene CG96736-01 was assessed using the primer-probe sets Ag3788 and Ag4075, described in Tables AUA and AUB. Results of the RTQ-PCR runs are shown in Tables AUC, AUD, AUE, AUF, AUG, AUH, AUI, AUJ and AUK.

TABLE AUA

Probe Name Ag3788

| | | | Start | SEQ ID |
|---|---|---|---|---|
| Primers | | Length | Position | No |
| Forward | 5′-cgagaaatatcttcccttccaa-3′ | 22 | 1182 | 432 |
| Probe | TET-5′-tgtcagcagcctttcgctcatactct-3′-TAMRA | 26 | 1209 | 433 |
| Reverse | 5′-ttccggtgatattcctctcttc-3′ | 22 | 1244 | 434 |

TABLE AUB

Probe Name Ag4075

| | | | Start | SEQ ID |
|---|---|---|---|---|
| Primers | | Length | Position | No |
| Forward | 5′-cgagaaatatcttcccttccaa-3′ | 22 | 1182 | 435 |
| Probe | TET-5′-tgtcagcagcctttcgctcatactct-3′-TAMRA | 26 | 1209 | 436 |
| Reverse | 5′-ttccggtgatattcctctcttc-3′ | 22 | 1244 | 437 |

TABLE AUC
AI_comprehensive panel_v1.0

| | Rel. | | Rel. |
|---|---|---|---|
| | Exp. (%) | | Exp. (%) |
| | Ag4075, | | Ag4075, |
| | Run | | Run |
| Tissue Name | 226203371 | issue Name | 226203371 |
| 110967 COPD-F | 6.0 | 112427 Match Control Psoriasis-F | 12.3 |
| 110980 COPD-F | 9.9 | 112418 Psoriasis-M | 3.6 |
| 110968 COPD-M | 6.6 | 112723 Match Control Psoriasis-M | 6.3 |
| 110977 COPD-M | 0.0 | 112419 Psoriasis-M | 6.5 |
| 110989 Emphysema-F | 8.7 | 112424 Match Control Psoriasis-M | 2.7 |
| 110992 Emphysema-F | 12.3 | 112420 Psoriasis-M | 14.1 |
| 110993 Emphysema-F | 7.2 | 112425 Match Control Psoriasis-M | 6.7 |
| 110994 Emphysema-F | 4.6 | 104689 (MF) OA Bone-Backus | 21.6 |
| 110995 Emphysema-F | 20.3 | 104690 (MF) Adj “Normal” | 21.8 |
| | | Bone-Backus | |
| 110996 Emphysema-F | 7.1 | 104691 (MF) OA Synovium-Backus | 14.1 |
| 110997 Asthma-M | 2.5 | 104692 (BA) OA Cartilage-Backus | 53.6 |
| 111001 Asthma-F | 6.7 | 104694 (BA) OA Bone-Backus | 14.8 |
| 111002 Asthma-F | 5.7 | 104695 (BA) Adj “Normal” | 28.7 |
| 111003 Atopic Asthma-F | 11.0 | 104696 (BA) OA Synovium-Backus | 15.8 |
| 111004 Atopic Asthma-F | 13.3 | 104700 (SS) OA Bone-Backus | 11.6 |
| 111005 Atopic Asthma-F | 12.2 | 104701 (SS) Adj “Normal” | 12.7 |
| | | Bone-Backus | |
| 111006 Atopic Asthma-F | 2.6 | 104702 (SS) OA Synovium-Backus | 27.5 |
| 111417 Allergy-M | 7.6 | 117093 OA Cartilage Rep7 | 6.3 |
| 112347 Allergy-M | 0.0 | 112672 OA Bone5 | 6.0 |
| 112349 Normal Lung-F | 0.0 | 112673 OA Synovium5 | 1.4 |
| 112357 Normal Lung-F | 19.9 | 112674 OA Synovial Fluid cells5 | 3.0 |
| 112354 Normal Lung-M | 4.0 | 117100 OA Cartilage Rep14 | 4.0 |
| 112374 Crohns-F | 2.7 | 112756 OA Bone9 | 100.0 |
| 112389 Match Control Crohns-F | 9.3 | 112757 OA Synovium9 | 0.9 |
| 112375 Crohns-F | 2.0 | 112758 OA Synovial Fluid Cells9 | 3.8 |
| 112732 Match Control Crohns-F | 12.6 | 117125 RA Cartilage Rep2 | 9.0 |
| 112725 Crohns-M | 0.3 | 113492 Bone2 RA | 8.1 |
| 112387 Match Control | 5.0 | 113493 Synovium2 RA | 2.5 |
| Crohns-M | | | |
| 112378 Crohns-M | 0.0 | 113494 Syn Fluid Cells RA | 5.3 |
| 112390 Match Control | 6.0 | 113499 Cartilage4 RA | 6.7 |
| Crohns-M | | | |
| 112726 Crohns-M | 9.9 | 113500 Bone4 RA | 7.0 |
| 112731 Match Control | 8.1 | 113501 Synovium4 RA | 4.4 |
| Crohns-M | | | |
| 112380 Ulcer Col-F | 6.0 | 113502 Syn Fluid Cells4 RA | 3.2 |
| 112734 Match Control Ulcer | 21.0 | 113495 Cartilage3 RA | 6.3 |
| Col-F | | | |
| 112384 Ulcer Col-F | 14.1 | 113496 Bone3 RA | 8.4 |
| 112737 Match Control Ulcer | 3.4 | 113497 Synovium3 RA | 5.1 |
| Col-F | | | |
| 112386 Ulcer Col-F | 3.4 | 113498 Syn Fluid Cells3 RA | 7.9 |
| 112738 Match Control Ulcer | 18.0 | 117106 Normal Cartilage Rep20 | 8.7 |
| Col-F | | | |
| 112381 Ulcer Col-M | 0.0 | 113663 Bone3 Normal | 0.0 |
| 112735 Match Control Ulcer | 0.5 | 113664 Synovium3 Normal | 0.0 |
| Col-M | | | |
| 112382 Ulcer Col-M | 7.1 | 113665 Syn Fluid Cells3 Normal | 0.0 |
| 112394 Match Control Ulcer | 1.6 | 117107 Normal Cartilage Rep22 | 1.8 |
| Col-M | | | |
| 112383 Ulcer Col-M | 13.1 | 113667 Bone4 Normal | 2.4 |
| 112736 Match Control Ulcer | 3.8 | 113668 Synovium4 Normal | 1.7 |
| Col-M | | | |
| 112423 Psoriasis-F | 6.3 | 113669 Syn Fluid Cells4 Normal | 3.9 |

TABLE AUD
CNS_neurodegeneration_v1.0

| | Rel. | | Rel. |
|---|---|---|---|
| | Exp. (%) | | Exp. (%) |
| | Ag4075, | | Ag4075, |
| | Run | | Run |
| Tissue Name | 214294982 | issue Name | 214294982 |
| AD 1 Hippo | 11.0 | Control (Path) 3 Temporal Ctx | 1.0 |
| AD 2 Hippo | 8.4 | Control (Path) 4 Temporal Ctx | 1.7 |
| AD 3 Hippo | 8.0 | AD 1 Occipital Ctx | 6.5 |
| AD 4 Hippo | 2.9 | AD 2 Occipital Ctx (Missing) | 0.0 |
| AD 5 Hippo | 16.8 | AD 3 Occipital Ctx | 1.3 |
| AD 6 Hippo | 100.0 | AD 4 Occipital Ctx | 3.6 |
| Control 2 Hippo | 19.6 | AD 5 Occipital Ctx | 11.9 |
| Control 4 Hippo | 17.6 | AD 6 Occipital Ctx | 6.5 |
| Control (Path) 3 Hippo | 3.0 | Control 1 Occipital Ctx | 5.6 |
| AD 1 Temporal Ctx | 6.3 | Control 2 Occipital Ctx | 10.4 |
| AD 2 Temporal Ctx | 14.1 | Control 3 Occipital Ctx | 6.0 |
| AD 3 Temporal Ctx | 4.2 | Control 4 Occipital Ctx | 2.9 |
| AD 4 Temporal Ctx | 7.5 | Control (Path) 1 Occipital Ctx | 3.3 |
| AD 5 Inf Temporal Ctx | 8.9 | Control (Path) 2 Occipital Ctx | 0.5 |
| AD 5 Sup Temporal Ctx | 24.5 | Control (Path) 3 Occipital Ctx | 1.6 |
| AD 6 Inf Temporal Ctx | 78.5 | Control (Path) 4 Occipital Ctx | 0.4 |
| AD 6 Sup Temporal Ctx | 56.6 | Control 1 Parietal Ctx | 5.9 |
| Control 1 Temporal Ctx | 2.3 | Control 2 Parietal Ctx | 9.9 |
| Control 2 Temporal Ctx | 12.1 | Control 3 Parietal Ctx | 6.0 |
| Control 3 Temporal Ctx | 7.7 | Control (Path) 1 Parietal Ctx | 3.6 |
| Control 3 Temporal Ctx | 3.1 | Control (Path) 2 Parietal Ctx | 1.1 |
| Control (Path) 1 Temporal Ctx | 4.6 | Control (Path) 3 Parietal Ctx | 2.2 |
| Control (Path) 2 Temporal Ctx | 1.8 | Control (Path) 4 Parietal Ctx | 3.4 |

TABLE AUE
General_screening_panel_v1.4

| | Rel. | Rel. | | Rel. | Rel. |
|---|---|---|---|---|---|
| | Exp. (%) | Exp. (%) | | Exp. (%) | Exp. (%) |
| | Ag4075, | Ag4075, | | Ag4075, | Ag4075, |
| | Run | Run | | Run | Run |
| Tissue Name | 212696066 | 218525356 | Tissue Name | 212696066 | 218525356 |
| Adipose | 0.0 | 1.3 | Renal ca. TK-10 | 9.7 | 14.8 |
| Melanoma* | 14.4 | 23.2 | Bladder | 1.0 | 1.8 |
| Hs688(A).T | | | | | |
| Melanoma* | 19.1 | 29.9 | Gastric ca. (liver | 41.5 | 42.0 |
| Hs688(B).T | | | met.) NCI-N87 | | |
| Melanoma* M14 | 9.5 | 12.7 | Gastric ca. KATO | 25.5 | 22.8 |
| | | | III | | |
| Melanoma* | 8.1 | 12.9 | Colon ca. SW-948 | 4.4 | 5.6 |
| LOXIMVI | | | | | |
| Melanoma* | 5.9 | 14.2 | Colon ca. SW480 | 100.0 | 100.0 |
| SK-MEL-5 | | | | | |
| Squamous cell | 5.1 | 10.2 | Colon ca.* (SW480 | 41.5 | 50.0 |
| carcinoma SCC-4 | | | met) SW620 | | |
| Testis Pool | 1.4 | 1.9 | Colon ca. HT29 | 10.2 | 13.6 |
| Prostate ca.* (bone | 9.5 | 13.6 | Colon ca. HCT-116 | 13.0 | 20.9 |
| met) PC-3 | | | | | |
| Prostate Pool | 1.1 | 1.5 | Colon ca. CaCo-2 | 12.0 | 14.5 |
| Placenta | 1.1 | 1.3 | Colon cancer tissue | 5.0 | 8.4 |
| Uterus Pool | 0.1 | 0.2 | Colon ca. SW1116 | 14.7 | 15.9 |
| Ovarian ca. | 6.5 | 8.0 | Colon ca. Colo-205 | 24.7 | 29.5 |
| OVCAR-3 | | | | | |
| Ovarian ca. SK-OV-3 | 8.1 | 9.9 | Colon ca. SW-48 | 3.6 | 4.7 |
| Ovarian ca. | 9.2 | 16.4 | Colon Pool | 0.7 | 1.1 |
| OVCAR-4 | | | | | |
| Ovarian ca. | 28.1 | 32.1 | Small Intestine Pool | 0.5 | 0.6 |
| OVCAR-5 | | | | | |
| Ovarian ca. | 23.0 | 33.2 | Stomach Pool | 0.8 | 0.8 |
| IGROV-1 | | | | | |
| Ovarian ca. | 10.3 | 16.4 | Bone Marrow Pool | 0.2 | 0.4 |
| OVCAR-8 | | | | | |
| Ovary | 0.5 | 0.8 | Fetal Heart | 0.1 | 0.1 |
| Breast ca. MCF-7 | 15.7 | 17.2 | Heart Pool | 0.2 | 0.3 |
| Breast ca. | 10.4 | 15.6 | Lymph Node Pool | 1.2 | 1.0 |
| MDA-MB-231 | | | | | |
| Breast ca. BT 549 | 9.9 | 18.7 | Fetal Skeletal | 0.2 | 0.2 |
| | | | Muscle | | |
| Breast ca. T47D | 53.2 | 51.8 | Skeletal Muscle | 0.2 | 0.3 |
| | | | Pool | | |
| Breast ca. MDA-N | 4.7 | 6.3 | Spleen Pool | 0.7 | 0.5 |
| Breast Pool | 0.6 | 0.6 | Thymus Pool | 0.8 | 0.9 |
| Trachea | 3.6 | 5.3 | CNS cancer | 20.0 | 20.3 |
| | | | (glio/astro) | | |
| | | | U87-MG | | |
| Lung | 0.1 | 0.1 | CNS cancer | 11.2 | 12.9 |
| | | | (glio/astro) | | |
| | | | U-118-MG | | |
| Fetal Lung | 2.4 | 4.0 | CNS cancer | 6.9 | 8.9 |
| | | | (neuro; met) | | |
| | | | SK-N-AS | | |
| Lung ca. NCI-N417 | 1.6 | 0.0 | CNS cancer (astro) | 9.3 | 12.0 |
| | | | SF-539 | | |
| Lung ca. LX-1 | 81.8 | 82.4 | CNS cancer (astro) | 36.1 | 55.5 |
| | | | SNB-75 | | |
| Lung ca. NCI-H146 | 0.4 | 0.8 | CNS cancer (glio) | 30.1 | 37.6 |
| | | | SNB-19 | | |
| Lung ca. SHP-77 | 6.8 | 8.5 | CNS cancer (glio) | 58.6 | 60.7 |
| | | | SF-295 | | |
| Lung ca. A549 | 9.8 | 15.8 | Brain (Amygdala) | 0.0 | 0.1 |
| | | | Pool | | |
| Lung ca. NCI-H526 | 2.1 | 2.5 | Brain (cerebellum) | 0.1 | 0.2 |
| Lung ca. NCI-H23 | 4.3 | 4.2 | Brain (fetal) | 0.2 | 0.3 |
| Lung ca. NCI-H460 | 9.2 | 16.2 | Brain | 0.1 | 0.1 |
| | | | (Hippocampus) | | |
| | | | Pool | | |
| Lung ca. HOP-62 | 4.4 | 4.5 | Cerebral Cortex | 0.0 | 0.1 |
| | | | Pool | | |
| Lung ca. NCI-H522 | 9.5 | 10.0 | Brain (Substantia | 0.1 | 0.1 |
| | | | nigra) Pool | | |
| Liver | 0.0 | 0.1 | Brain (Thalamus) | 0.0 | 0.1 |
| | | | Pool | | |
| Fetal Liver | 2.9 | 4.3 | Brain (whole) | 0.2 | 0.2 |
| Liver ca. HepG2 | 6.7 | 7.9 | Spinal Cord Pool | 0.2 | 0.3 |
| Kidney Pool | 1.1 | 1.2 | Adrenal Gland | 0.3 | 0.6 |
| Fetal Kidney | 0.3 | 0.5 | Pituitary gland Pool | 0.1 | 0.3 |
| Renal ca. 786-0 | 5.1 | 9.5 | Salivary Gland | 3.0 | 2.8 |
| Renal ca. A498 | 3.1 | 5.0 | Thyroid (female) | 0.1 | 0.1 |
| Renal ca. ACHN | 5.1 | 5.9 | Pancreatic ca. | 7.9 | 12.2 |
| | | | CAPAN2 | | |
| Renal ca. UO-31 | 2.6 | 4.2 | Pancreas Pool | 1.3 | 1.2 |

TABLE AUF
General_screening_panel_v1.5

| | Rel. | | Rel. |
|---|---|---|---|
| | Exp. (%) | | Exp. (%) |
| | Ag4075, | | Ag4075, |
| | Run | | Run |
| Tissue Name | 228714883 | issue Name | 228714883 |
| Adipose | 1.0 | Renal ca. TK-10 | 9.8 |
| Melanoma* Hs688(A).T | 18.0 | Bladder | 1.4 |
| Melanoma* Hs688(B).T | 17.4 | Gastric ca. (liver met.) NCI-N87 | 35.4 |
| Melanoma* M14 | 9.5 | Gastric ca. KATO III | 19.9 |
| Melanoma* LOXIMVI | 9.0 | Colon ca. SW-948 | 4.4 |
| Melanoma* SK-MEL-5 | 8.7 | Colon ca. SW480 | 100.0 |
| Squamous cell carcinoma SCC-4 | 5.8 | Colon ca.* (SW480 met) SW620 | 32.8 |
| Testis Pool | 1.2 | Colon ca. HT29 | 9.9 |
| Prostate ca.* (bone met) PC-3 | 10.8 | Colon ca. HCT-116 | 15.2 |
| Prostate Pool | 1.5 | Colon ca. CaCo-2 | 11.1 |
| Placenta | 1.1 | Colon cancer tissue | 5.1 |
| Uterus Pool | 0.3 | Colon ca. SW1116 | 7.2 |
| Ovarian ca. OVCAR-3 | 6.2 | Colon ca. Colo-205 | 23.7 |
| Ovarian ca. SK-OV-3 | 7.5 | Colon ca. SW-48 | 3.2 |
| Ovarian ca. OVCAR-4 | 12.5 | Colon Pool | 0.7 |
| Ovarian ca. OVCAR-5 | 20.2 | Small Intestine Pool | 0.4 |
| Ovarian ca. IGROV-1 | 23.8 | Stomach Pool | 0.7 |
| Ovarian ca. OVCAR-8 | 11.2 | Bone Marrow Pool | 0.2 |
| Ovary | 0.6 | Fetal Heart | 0.1 |
| Breast ca. MCF-7 | 14.4 | Heart Pool | 0.2 |
| Breast ca. MDA-MB-231 | 14.1 | Lymph Node Pool | 0.7 |
| Breast ca. BT 549 | 8.4 | Fetal Skeletal Muscle | 0.2 |
| Breast ca. T47D | 2.1 | Skeletal Muscle Pool | 0.4 |
| Breast ca. MDA-N | 3.6 | Spleen Pool | 0.3 |
| Breast Pool | 0.5 | Thymus Pool | 0.5 |
| Trachea | 4.6 | CNS cancer (glio/astro) U87-MG | 12.5 |
| Lung | 0.1 | CNS cancer (glio/astro) U-118-MG | 8.5 |
| Fetal Lung | 2.6 | CNS cancer (neuro; met) SK-N-AS | 5.5 |
| Lung ca. NCI-N417 | 1.9 | CNS cancer (astro) SF-539 | 8.4 |
| Lung ca. LX-1 | 81.8 | CNS cancer (astro) SNB-75 | 13.1 |
| Lung ca. NCI-H146 | 0.6 | CNS cancer (glio) SNB-19 | 27.2 |
| Lung ca. SHP-77 | 7.7 | CNS cancer (glio) SF-295 | 53.2 |
| Lung ca. A549 | 11.8 | Brain (Amygdala) Pool | 0.0 |
| Lung ca. NCI-H526 | 2.1 | Brain (cerebellum) | 0.1 |
| Lung ca. NCI-H23 | 3.5 | Brain (fetal) | 0.2 |
| Lung ca. NCI-H460 | 8.8 | Brain (Hippocampus) Pool | 0.0 |
| Lung ca. HOP-62 | 3.5 | Cerebral Cortex Pool | 0.1 |
| Lung ca. NCI-H522 | 7.5 | Brain (Substantia nigra) Pool | 0.1 |
| Liver | 0.0 | Brain (Thalamus) Pool | 0.1 |
| Fetal Liver | 2.9 | Brain (whole) | 0.2 |
| Liver ca. HepG2 | 6.2 | Spinal Cord Pool | 0.1 |
| Kidney Pool | 0.8 | Adrenal Gland | 0.4 |
| Fetal Kidney | 0.3 | Pituitary gland Pool | 0.2 |
| Renal ca. 786-0 | 5.6 | Salivary Gland | 2.7 |
| Renal ca. A498 | 3.4 | Thyroid (female) | 0.1 |
| Renal ca. ACHN | 4.9 | Pancreatic ca. CAPAN2 | 9.7 |
| Renal ca. UO-31 | 2.4 | Pancreas Pool | 0.8 |

TABLE AUG
Panel 3D

| | Rel. | | Rel. |
|---|---|---|---|
| | Exp. () | | Exp. (%) |
| | Ag4075, | | Ag4075, |
| | Run | | Run |
| Tissue Name | 186579982 | Tissue Name | 186579982 |
| Daoy-Medulloblastoma | 1.7 | Ca Ski-Cervical epidermoid | 9.3 |
| | | carcinoma (metastasis) | |
| TE671-Medulloblastoma | 1.3 | ES-2-Ovarian clear cell carcinoma | 4.2 |
| D283 Med-Medulloblastoma | 13.6 | Ramos-Stimulated with | 12.2 |
| | | PMA/ionomycin 6 h | |
| PFSK-1-Primitive | 8.0 | Ramos-Stimulated with | 12.2 |
| Neuroectodermal | | PMA/ionomycin 14 h | |
| XF-498-CNS | 5.1 | MEG-01-Chronic myelogenous | 25.0 |
| | | leukemia (megokaryoblast) | |
| SNB-78-Glioma | 12.9 | Raji-Burkitt's lymphoma | 2.4 |
| SF-268-Glioblastoma | 5.4 | Daudi-Burkitt's lymphoma | 5.0 |
| T98G-Glioblastoma | 7.9 | U266-B-cell plasmacytoma | 9.3 |
| SK-N-SH-Neuroblastoma | 4.4 | CA46-Burkitt's lymphoma | 2.6 |
| (metastasis) | | | |
| SF-295-Glioblastoma | 8.2 | RL-non-Hodgkin's B-cell | 6.5 |
| | | lymphoma | |
| Cerebellum | 0.1 | JM1-pre-B-cell lymphoma | 6.0 |
| Cerebellum | 0.1 | Jurkat-T cell leukemia | 7.6 |
| NCI-H292-Mucoepidermoid | 12.0 | TF-1-Erythroleukemia | 17.6 |
| lung carcinoma | | | |
| DMS-114-Small cell lung cancer | 3.0 | HUT 78-T-cell lymphoma | 4.9 |
| DMS-79-Small cell lung cancer | 92.0 | U937-Histiocytic lymphoma | 17.9 |
| NCI-H146-Small cell lung | 1.6 | KU-812-Myelogenous leukemia | 15.4 |
| cancer | | | |
| NCI-H526-Small cell lung | 10.7 | 769-P-Clear cell renal carcinoma | 5.8 |
| cancer | | | |
| NCI-N417-Small cell lung | 3.0 | Caki-2-Clear cell renal carcinoma | 5.5 |
| cancer | | | |
| NCI-H82-Small cell lung cancer | 5.7 | SW 839-Clear cell renal carcinoma | 6.2 |
| NCI-H157-Squamous cell lung | 30.1 | G401-Wilms' tumor | 3.8 |
| cancer (metastasis) | | | |
| NCI-H1155-Large cell lung | 9.5 | Hs766T-Pancreatic carcinoma (LN | 7.6 |
| cancer | | metastasis) | |
| NCI-H1299-Large cell lung | 6.1 | CAPAN-1-Pancreatic | 3.3 |
| cancer | | adenocarcinoma (liver metastasis) | |
| NCI-H727-Lung carcinoid | 8.7 | SU86.86-Pancreatic carcinoma | 5.1 |
| | | (liver metastasis) | |
| NCI-UMC-11-Lung carcinoid | 14.4 | BxPC-3-Pancreatic | 11.4 |
| | | adenocarcinoma | |
| LX-1-Small cell lung cancer | 100.0 | HPAC-Pancreatic adenocarcinoma | 6.1 |
| Colo-205-Colon cancer | 49.3 | MIA PaCa-2-Pancreatic carcinoma | 1.1 |
| KM12-Colon cancer | 12.7 | CFPAC-1-Pancreatic ductal | 10.4 |
| | | adenocarcinoma | |
| KM20L2-Colon cancer | 11.7 | PANC-1-Pancreatic epithelioid | 4.3 |
| | | ductal carcinoma | |
| NCI-H716-Colon cancer | 10.2 | T24-Bladder carcinma (transitional | 1.5 |
| | | cell) | |
| SW-48-Colon adenocarcinoma | 6.7 | 5637-Bladder carcinoma | 2.8 |
| SW1116-Colon adenocarcinoma | 20.9 | HT-1197-Bladder carcinoma | 10.4 |
| LS 174T-Colon adenocarcinoma | 13.4 | UM-UC-3-Bladder carcinma | 1.4 |
| | | (transitional cell) | |
| SW-948-Colon adenocarcinoma | 0.9 | A204-Rhabdomyosarcoma | 2.6 |
| SW-480-Colon adenocarcinoma | 3.5 | HT-1080-Fibrosarcoma | 4.7 |
| NCI-SNU-5-Gastric carcinoma | 34.6 | MG-63-Osteosarcoma | 8.1 |
| KATO III-Gastric carcinoma | 38.7 | SK-LMS-1-Leiomyosarcoma | 8.1 |
| | | (vulva) | |
| NCI-SNU-16-Gastric carcinoma | 2.9 | SJRH30-Rhabdomyosarcoma (met | 1.9 |
| | | to bone marrow) | |
| NCI-SNU-1-Gastric carcinoma | 22.4 | A431-Epidermoid carcinoma | 10.6 |
| RF-1-Gastric adenocarcinoma | 1.8 | WM266-4-Melanoma | 5.5 |
| RF-48-Gastric adenocarcinoma | 1.9 | DU 145-Prostate carcinoma (brain | 0.1 |
| | | metastasis) | |
| MKN-45-Gastric carcinoma | 12.0 | MDA-MB-468-Breast | 13.4 |
| | | adenocarcinoma | |
| NCI-N87-Gastric carcinoma | 24.5 | SCC-4-Squamous cell carcinoma of | 0.2 |
| | | tongue | |
| OVCAR-5-Ovarian carcinoma | 2.3 | SCC-9-Squamous cell carcinoma of | 0.2 |
| | | tongue | |
| RL95-2-Uterine carcinoma | 8.3 | SCC-15-Squamous cell carcinoma | 0.3 |
| | | of tongue | |
| HelaS3-Cervical | 2.3 | CAL 27-Squamous cell carcinoma | 10.7 |
| adenocarcinoma | | of tongue | |

TABLE AUH
Panel 4.1D

| | Rel. | | Rel. |
|---|---|---|---|
| | Exp. (% | | Exp. (%) |
| | Ag4075, | | Ag4075, |
| | Run | | Run |
| Tissue Name | 184565261 | Tissue Name | 184565261 |
| Secondary Th1 act | 81.2 | HUVEC IL-1beta | 35.1 |
| Secondary Th2 act | 84.1 | HUVEC IFN gamma | 17.6 |
| Secondary Tr1 act | 67.8 | HUVEC TNF alpha + IFN gamma | 24.7 |
| Secondary Th1 rest | 3.5 | HUVEC TNF alpha + IL4 | 29.9 |
| Secondary Th2 rest | 11.3 | HUVEC IL-11 | 12.4 |
| Secondary Tr1 rest | 3.6 | Lung Microvascular EC none | 33.4 |
| Primary Th1 act | 43.2 | Lung Microvascular EC TNF alpha + IL-1beta | 21.0 |
| Primary Th2 act | 55.1 | Microvascular Dermal EC none | 20.3 |
| Primary Tr1 act | 51.8 | Microsvasular Dermal EC | 11.7 |
| | | TNF alpha + IL-1beta | |
| Primary Th1 rest | 3.3 | Bronchial epithelium TNF alpha + IL1beta | 39.8 |
| Primary Th2 rest | 2.2 | Small airway epithelium none | 10.8 |
| Primary Tr1 rest | 10.3 | Small airway epithelium TNF alpha + IL-1beta | 15.3 |
| CD45RA CD4 lymphocyte act | 52.5 | Coronery artery SMC rest | 34.6 |
| CD45RO CD4 lymphocyte act | 45.7 | Coronery artery SMC TNF alpha + IL-1beta | 32.5 |
| CD8 lymphocyte act | 51.1 | Astrocytes rest | 10.9 |
| Secondary CD8 lymphocyte rest | 41.5 | Astrocytes TNF alpha + IL-1beta | 7.1 |
| Secondary CD8 lymphocyte act | 36.1 | KU-812 (Basophil) rest | 52.1 |
| CD4 lymphocyte none | 0.6 | KU-812 (Basophil) | 82.4 |
| | | PMA/ionomycin | |
| 2ry Th1/Th2/Tr1_anti-CD95 | 4.0 | CCD1106 (Keratinocytes) none | 52.9 |
| CH11 | | | |
| LAK cells rest | 24.1 | CCD1106 (Keratinocytes) | 39.8 |
| | | TNF alpha + IL-1beta | |
| LAK cells IL-2 | 34.6 | Liver cirrhosis | 2.8 |
| LAK cells IL-2 + IL-12 | 28.3 | NCI-H292 none | 27.0 |
| LAK cells IL-2 + IFN gamma | 20.4 | NCI-H292 IL-4 | 53.6 |
| LAK cells IL-2 + IL-18 | 29.5 | NCI-H292 IL-9 | 29.5 |
| LAK cells PMA/ionomycin | 49.0 | NCI-H292 IL-13 | 51.4 |
| NK Cells IL-2 rest | 43.2 | NCI-H292 IFN gamma | 58.6 |
| Two Way MLR 3 day | 22.4 | HPAEC none | 10.4 |
| Two Way MLR 5 day | 39.8 | HPAEC TNF alpha + IL-1beta | 17.0 |
| Two Way MLR 7 day | 25.9 | Lung fibroblast none | 42.0 |
| PBMC rest | 2.3 | Lung fibroblast TNF alpha + IL-1 | 17.7 |
| | | beta | |
| PBMC PWM | 42.3 | Lung fibroblast IL-4 | 36.6 |
| PBMC PHA-L | 30.1 | Lung fibroblast IL-9 | 38.4 |
| Ramos (B cell) none | 57.4 | Lung fibroblast IL-13 | 41.2 |
| Ramos (B cell) ionomycin | 100.0 | Lung fibroblast IFN gamma | 39.5 |
| B lymphocytes PWM | 31.2 | Dermal fibroblast CCD1070 rest | 84.7 |
| B lymphocytes CD40L and IL-4 | 14.5 | Dermal fibroblast CCD1070 TNF | 59.0 |
| | | alpha | |
| EOL-1 dbcAMP | 61.1 | Dermal fibroblast CCD1070 IL-1 | 55.1 |
| | | beta | |
| EOL-1 dbcAMP | 21.2 | Dermal fibroblast IFN gamma | 16.7 |
| PMA/ionomycin | | | |
| Dendritic cells none | 28.5 | Dermal fibroblast IL-4 | 36.9 |
| Dendritic cells LPS | 7.9 | Dermal fibroblast rest | 15.0 |
| Dendritic cells anti-CD40 | 32.8 | Neutrophils TNFa + LPS | 1.6 |
| Monocytes rest | 11.0 | Neutrophils rest | 0.4 |
| Monocytes LPS | 5.4 | Colon | 4.5 |
| Macrophages rest | 25.5 | Lung | 7.5 |
| Macrophages LPS | 3.7 | Thymus | 6.3 |
| HUVEC none | 21.9 | Kidney | 12.9 |
| HUVEC starved | 27.7 | | |

TABLE AUI
Panel 5 Islet

| | Rel. | | Rel. |
|---|---|---|---|
| | Exp. (%) | | Exp. (%) |
| | Ag4075, | | Ag4075, |
| | Run | | Run |
| Tissue Name | 186511155 | Tissue Name | 186511155 |
| 97457_Patient-02go_adipose | 7.6 | 94709_Donor 2 AM - A_adipose | 45.7 |
| 97476_Patient-07sk_skeletal | 2.9 | 94710_Donor 2 AM - B_adipose | 27.4 |
| muscle | | | |
| 97477_Patient-07ut_uterus | 3.5 | 94711_Donor 2 AM - C_adipose | 15.2 |
| 97478_Patient-07pl_placenta | 5.0 | 94712_Donor 2 AD - A_adipose | 62.9 |
| 99167_Bayer Patient 1 | 30.6 | 94713_Donor 2 AD - B_adipose | 66.4 |
| 97482_Patient-08ut_uterus | 4.6 | 94714_Donor 2 AD - C_adipose | 57.4 |
| 97483_Patient-08pl_placenta | 3.8 | 94742_Donor 3 U - A_Mesenchymal | 36.1 |
| | | Stem Cells | |
| 97486_Patient-09sk_skeletal | 0.3 | 94743_Donor 3 U - B_Mesenchymal | 62.4 |
| muscle | | Stem Cells | |
| 97487_Patient-09ut_uterus | 8.3 | 94730_Donor 3 AM - A_adipose | 34.9 |
| 97488_Patient-09pl_placenta | 3.4 | 94731_Donor 3 AM - B_adipose | 17.2 |
| 97492_Patient-10ut_uterus | 7.5 | 94732_Donor 3 AM - C_adipose | 22.4 |
| 97493_Patient-10pl_placenta | 5.1 | 94733_Donor 3 AD - A_adipose | 100.0 |
| 97495_Patient-11go_adipose | 6.4 | 94734_Donor 3 AD - B_adipose | 32.3 |
| 97496_Patient-11sk_skeletal | 1.3 | 94735_Donor 3 AD - C_adipose | 66.9 |
| muscle | | | |
| 97497_Patient-11ut_uterus | 11.6 | 77138_Liver_HepG2untreated | 31.4 |
| 97498_Patient-11pl_placenta | 3.9 | 73556_Heart_Cardiac stromal cells | 3.6 |
| | | (primary) | |
| 97500_Patient-12go_adipose | 8.5 | 81735_Small Intestine | 6.4 |
| 97501_Patient-12sk_skeletal | 2.7 | 72409_Kidney_Proximal Convoluted | 3.8 |
| muscle | | Tubule | |
| 97502_Patient-12ut_uterus | 8.7 | 82685_Small intestine_Duodenum | 1.9 |
| 97503_Patient-12pl_placenta | 3.1 | 90650_Adrenal_Adrenocortical | 1.4 |
| | | adenoma | |
| 94721_Donor 2 U - | 40.1 | 72410_Kidney_HRCE | 14.9 |
| A_Mesenchymal Stem Cells | | | |
| 94722_Donor 2 U - | 23.7 | 72411_Kidney_HRE | 11.1 |
| B_Mesenchymal Stem Cells | | | |
| 94723_Donor 2 U - | 52.5 | 73139_Uterus_Uterine smooth | 17.4 |
| C_Mesenchymal Stem Cells | | muscle cells | |

TABLE AUJ
Panel 5D

| | Rel. | Rel. | | Rel. | Rel. |
|---|---|---|---|---|---|
| | Exp. (%) | Exp. (%) | | Exp. (%) | Exp. (%) |
| | Ag378, | Ag4075, | | Ag3788, | Ag4075, |
| | Run | Run | | Run | Run |
| Tissue Name | 170222681 | 172167823 | Tissue Name | 170222681 | 172167823 |
| 97457_Patient-02go_adipose | 8.2 | 11.0 | 94709_Donor 2 AM - | 44.1 | 53.2 |
| | | | A_adipose | | |
| 97476_Patient-07sk_skeletal | 2.1 | 2.8 | 94710_Donor 2 AM - | 31.2 | 28.3 |
| muscle | | | B_adipose | | |
| 97477_Patient-07ut_uterus | 3.5 | 7.1 | 94711_Donor 2 AM - | 29.3 | 30.8 |
| | | | C_adipose | | |
| 97478_Patient-07pl_placenta | 5.1 | 5.8 | 94712_Donor 2 AD - | 77.4 | 81.8 |
| | | | A_adipose | | |
| 97481_Patient-08sk_skeletal | 4.2 | 3.9 | 94713_Donor 2 AD - | 100.0 | 100.0 |
| muscle | | | B_adipose | | |
| 97482_Patient-08ut_uterus | 5.7 | 8.7 | 94714_Donor 2 AD - | 68.8 | 84.1 |
| | | | C_adipose | | |
| 97483_Patient-08pl_placenta | 7.5 | 7.2 | 94742_Donor 3 U - | 55.1 | 66.9 |
| | | | A_Mesenchymal Stem Cells | | |
| 97486_Patient-09sk_skeletal | 0.9 | 1.2 | 94743_Donor 3 U - | 62.9 | 70.7 |
| muscle | | | B_Mesenchymal Stem Cells | | |
| 97487_Patient-09ut_uterus | 8.5 | 11.0 | 94730_Donor 3 AM - | 41.5 | 46.7 |
| | | | A_adipose | | |
| 97488_Patient-09pl_placenta | 4.9 | 4.2 | 94731_Donor 3 AM - | 29.7 | 29.5 |
| | | | B_adipose | | |
| 97492_Patient-10ut_uterus | 5.7 | 5.8 | 94732_Donor 3 AM - | 25.7 | 36.6 |
| | | | C_adipose | | |
| 97493_Patient-10pl_placenta | 6.3 | 7.0 | 94733_Donor 3 AD - | 97.3 | 92.7 |
| | | | A_adipose | | |
| 97495_Patient-11go_adipose | 7.3 | 8.8 | 94734_Donor 3 AD - | 58.6 | 80.7 |
| | | | B_adipose | | |
| 97496_Patient-11sk_skeletal | 1.7 | 1.3 | 94735_Donor 3 AD - | 69.3 | 83.5 |
| muscle | | | C_adipose | | |
| 97497_Patient-11ut_uterus | 12.9 | 15.7 | 77138_Liver_HepG2untreated | 72.7 | 80.7 |
| 97498_Patient-11pl_placenta | 4.7 | 6.8 | 73556_Heart_Cardiac stromal | 2.6 | 4.7 |
| | | | cells (primary) | | |
| 97500_Patient-12go_adipose | 9.5 | 12.6 | 81735_Small Intestine | 7.6 | 8.9 |
| 97501_Patient-12sk_skeletal | 2.7 | 2.4 | 72409_Kidney_Proximal | 4.6 | 4.3 |
| muscle | | | Convoluted Tubule | | |
| 97502_Patient-12ut_uterus | 9.3 | 10.7 | 82685_Small | 1.9 | 2.0 |
| | | | intestine_Duodenum | | |
| 97503_Patient-12pl_placenta | 3.0 | 3.1 | 90650_Adrenal_Adrenocortical | 1.4 | 1.1 |
| | | | adenoma | | |
| 94721_Donor 2 U - | 50.3 | 52.9 | 72410_Kidney_HRCE | 21.9 | 21.5 |
| A_Mesenchymal Stem | | | | | |
| Cells | | | | | |
| 94722_Donor 2 U - | 45.4 | 47.3 | 72411_Kidney_HRE | 15.7 | 0.0 |
| B_Mesenchymal Stem | | | | | |
| Cells | | | | | |
| 94723_Donor 2 U - | 52.1 | 45.4 | 73139_Uterus_Uterine | 23.7 | 28.3 |
| C_Mesenchymal Stem | | | smooth muscle cells | | |
| Cells | | | | | |

TABLE AUK
general oncology screening panel_v_2.4

| | Rel. | | Rel. |
|---|---|---|---|
| | Exp. (%) | | Exp. (%) |
| | Ag4075, | | Ag4075, |
| | Run | | Run |
| Tissue Name | 259745203 | Tissue Nme | 259745203 |
| Colon cancer 1 | 50.7 | Bladder cancer NAT 2 | 0.1 |
| Colon cancer NAT 1 | 13.5 | Bladder cancer NAT 3 | 0.0 |
| Colon cancer 2 | 47.0 | Bladder cancer NAT 4 | 0.1 |
| Colon cancer NAT 2 | 24.3 | Prostate adenocarcinoma 1 | 33.9 |
| Colon cancer 3 | 95.9 | Prostate adenocarcinoma 2 | 3.6 |
| Colon cancer NAT 3 | 16.2 | Prostate adenocarcinoma 3 | 26.4 |
| Colon malignant cancer 4 | 55.9 | Prostate adenocarcinoma 4 | 100.0 |
| Colon normal adjacent tissue 4 | 6.2 | Prostate cancer NAT 5 | 6.8 |
| Lung cancer 1 | 11.4 | Prostate adenocarcinoma 6 | 11.2 |
| Lung NAT 1 | 0.6 | Prostate adenocarcinoma 7 | 8.0 |
| Lung cancer 2 | 12.9 | Prostate adenocarcinoma 8 | 2.6 |
| Lung NAT 2 | 1.0 | Prostate adenocarcinoma 9 | 38.2 |
| Squamous cell carcinoma 3 | 62.0 | Prostate cancer NAT 10 | 0.6 |
| Lung NAT 3 | 1.1 | Kidney cancer 1 | 7.9 |
| metastatic melanoma 1 | 20.2 | KidneyNAT 1 | 2.9 |
| Melanoma 2 | 3.1 | Kidney cancer 2 | 28.1 |
| Melanoma 3 | 1.7 | Kidney NAT 2 | 8.5 |
| metastatic melanoma 4 | 57.0 | Kidney cancer 3 | 13.9 |
| metastatic melanoma 5 | 25.3 | Kidney NAT 3 | 2.1 |
| Bladder cancer 1 | 0.2 | Kidney cancer 4 | 9.6 |
| Bladder cancer NAT 1 | 0.0 | Kidney NAT 4 | 11.2 |
| Bladder cancer 2 | 11.7 | | |

AI_comprehensive panel_v1.0 Summary: Ag4075 Highest expression is seen in an osteoarthritic bone sample (CT=27.31). This gene is expressed at moderate to low levels in many samples on this panel. Please see Panel 4.1 for discussion of this gene in inflammation.

CNS_neurodegeneration_v1.0 Summary: Ag4075 This panel does not show differential expression of this gene in Alzheimer's disease. However, this profile confirms the expression of this gene at moderate levels in the brain. Please see Panel 1.4 for discussion of this gene in the central nervous system.

General_screen_panel_v1.4 Summary: Ag4075 Two experiments with the same probe and primer set produce results that are in excellent agreement. Highest expression is seen in a colon cancer cell line (CTs=21-22). Overall, expression of this gene appears to be highly associated with cancer cell line samples, with high levels oof expression in brain, colon, gastric, lung, breast, ovarian, and melanoma cancer cell lines. This expression profile suggests a role for this gene product in cell survivaland proliferation. This gene encodes a protein with homology to Neutral amino acid transporter 2. L type amino acid transporter 1 (LAT1) has been implicated in tumor growth and may play an important role in supplying nutrition to cells for cell proliferation (Ohkame, J Surg Oncol 2001 Dec;78(4):265-71; discussion 271-2). Thus, modulation of this gene product may be useful in the treatment of cancer.

Among tissues with metabolic function, this gene is expressed at moderate levels in pituitary, adipose, adrenal gland, pancreas, thyroid, and adult and fetal skeletal muscle, heart, and liver. This widespread expression among these tissues suggests that this gene product may play a role in normal neuroendocrine and metabolic function and that disregulated expression of this gene may contribute to neuroendocrine disorders or metabolic diseases, such as obesity and diabetes.

This gene is also expressed at moderate levels in the CNS, including the hippocampus, thalamus, substantia nigra, amygdala, cerebellum and cerebral cortex. Therefore, therapeutic modulation of the expression or function of this gene may be useful in the treatment of neurologic disorders, such as Alzheimer's disease, Parkinson's disease, schizophrenia, multiple sclerosis, stroke and epilepsy.

In addition, this gene is expressed at much higher levels in fetal lung and liver tissue (CTs=26-27) when compared to expression in the adult counterparts (CTs=31-33). Thus, expression of this gene may be used to differentiate between the fetal and adult sources of these tissues.

General_screen_panel_v1.5 Summary: Ag4075 Highest expression is seen in a colon cancer cell line (CT=20), with expression in this panel in strong agreement with Panel 1.4. Please see that panel for discussion of this gene in disease.

Panel 3D Summary: Ag4075 Expression of this gene is widespread on this panel, with highest expression in a lung cancer cell line (CT=26). The widespread expression on this panel is in agreement with expression in Panels 1.4 and 1.5 where expression of this gene is highly associated with cancer cell line samples. Please see Panel 1.4 for discussion of this gene in oncology.

Panel 4.1D Summary: Ag4075 Highest expression of this gene is seen in a sample derived from the Ramos B cell line treated with ionomycin (CT=27.3). In addition, this gene appears to be more highly expressed in activated T cells than in resting T cells. Thus, therapeutic regulation of the transcript or the protein encoded by the transcript could be important in immune modulation and in the treatment of T cell-mediated diseases such as asthma, arthritis, psoriasis, IBD, and lupus. In addition, this gene is also expressed at moderate levels in a wide range of cell types of significance in the immune response in health and disease. These cells include members of the T-cell, B-cell, endothelial cell, macrophage/monocyte, and peripheral blood mononuclear cell family, as well as epithelial and fibroblast cell types from lung and skin, and normal tissues represented by colon, lung, thymus and kidney. This ubiquitous pattern of expression suggests that this gene product may be involved in homeostatic processes for these and other cell types and tissues. This pattern is in agreement with the expression profile in General_screen_panel_v1.4 and also suggests a role for the gene product in cell survival and proliferation. Therefore, modulation of the gene product with a functional therapeutic may lead to the alteration of functions associated with these cell types and lead to improvement of the symptoms of patients suffering from autoimmune and inflammatory diseases such as asthma, allergies, inflammatory bowel disease, lupus erythematosus, psoriasis, rheumatoid arthritis, and osteoarthritis.

Panel 5 Islet Summary: Ag4075 Highest expression is seen in adipose (CT=27). In addition, this expression of this gene is widespread on this panel, with moderate to high levels in metabolic tissues, including skeletal muscle, adipose, pancreatic islet cells and placenta. This gene codes for neutral amino acid transporter B(0)[ATB(0)]. ATB(0) transports the gluconeogenic amino acids 1-alanine and 1-glutamine into cells. Excess neutral amino acid transport and a resultant increase in gluconeogenesis and triglyceride synthesis may impair beta cell function in obesity and Type 2 diabetes. Pharmacologic inhibition of ATB(0) encoded by this gene may prevent or treat the symptoms of obesity-related Type 2 diabetes.

Panel 5D Summary: Ag4075 Expression on this panel agrees with Panel 5I. Highest expression is seen in adipose in two replicate experiments (CTs=28). Please see Panel 5I and 1.4 for further discussion of utility of this gene in metabolic disease.

General oncology screening panel_v_{—}2.4 Summary: Ag4975 Highest expression of this gene is seen in prostate cancer (CT=27). Prominent expression is also seen in melanoma and squamous cell carcinoma derived samples. In addition, this gene appears to be overexpressed in colon, lung, prostate cancer when compared to expression in the normal adjacent tissue. Thus, expression of this gene could be used as a marker to detect the presence of colon, lung and prostate cancer. Furthermore, therapeutic modulation of the expression or function of this gene may be effective in the treatment of colon, prostate, melanoma and lung cancer.

### Example D: Identification of Single Nucleotide Polymorphisms in NOVX Nucleic Acid Sequences

Variant sequences are also included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, when a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern. Examples include alteration in temporal expression, physiological response regulation, cell type expression regulation, intensity of expression, and stability of transcribed message.

SeqCalling assemblies produced by the exon linking process were selected and extended using the following criteria. Genomic clones having regions with 98% identity to all or part of the initial or extended sequence were identified by BLASTN searches using the relevant sequence to query human genomic databases. The genomic clones that resulted were selected for further analysis because this identity indicates that these clones contain the genomic locus for these SeqCalling assemblies. These sequences were analyzed for putative coding regions as well as for similarity to the known DNA and protein sequences. Programs used for these analyses include Grail, Genscan, BLAST, HMMER, FASTA, Hybrid and other relevant programs.

Some additional genomic regions may have also been identified because selected SeqCalling assemblies map to those regions. Such SeqCalling sequences may have overlapped with regions defined by homology or exon prediction. They may also be included because the location of the fragment was in the vicinity of genomic regions identified by similarity or exon prediction that had been included in the original predicted sequence. The sequence so identified was manually assembled and then may have been extended using one or more additional sequences taken from CuraGen Corporation's human SeqCalling database. SeqCalling fragments suitable for inclusion were identified by the CuraTool™ program SeqExtend or by identifying SeqCalling fragments mapping to the appropriate regions of the genomic clones analyzed.

The regions defined by the procedures described above were then manually integrated and corrected for apparent inconsistencies that may have arisen, for example, from miscalled bases in the original fragments or from discrepancies between predicted exon junctions, EST locations and regions of sequence similarity, to derive the final sequence disclosed herein. When necessary, the process to identify and analyze SeqCalling assemblies and genomic clones was reiterated to derive the full length sequence (Alderborn et al., Determination of Single Nucleotide Polymorphisms by Real-time Pyrophosphate DNA Sequencing. Genome Research. 10 (8) 1249-1265, 2000).

Variants are reported individually but any combination of all or a select subset of variants are also included as contemplated NOVX embodiments of the invention.

NOV1a SNP Data:

NOV1a has one SNP variant, whose variant positions for its nucleotide and amino acid sequences is numbered according to SEQ ID NOs: 1 and 2, respectively. The nucleotide sequence of the NOVIa variant differs as shown in Table SNP1.

TABLE SNP1

| | Nucleotides | | | Amino Acids | | |
|---|---|---|---|---|---|---|
| Variant | Position | Initial | Modified | Position | Initial | Modified |
| 13375555 | 4319 | C | T | 1440 | Pro | Leu |

NOV2b SNP Data:

NOV2b has six SNP variants, whose variant positions for its nucleotide and amino acid sequences are numbered according to SEQ ID NOs: 17 and 18, respectively. The nucleotide sequence of the NOV2b variant differs as shown in Table SNP2.

TABLE SNP2

| | Nucleotides | | | Amino Acids | | |
|---|---|---|---|---|---|---|
| Variant | Position | Initial | Modified | Position | Initial | Modified |
| 12252060 | 100 | A | T | 34 | Ile | Phe |
| 13380837 | 204 | A | C | 68 | Thr | Thr |
| 13380838 | 209 | G | A | 70 | Gly | Asp |
| 13380839 | 254 | A | G | 85 | Gln | Arg |
| 13380843 | 605 | C | T | 202 | Ala | Val |
| 13380844 | 614 | C | T | 205 | Ala | Val |

NOV3b SNP Data:

NOV3b has seven SNP variants, whose variant positions for its nucleotide and amino acid sequences are numbered according to SEQ ID NOs: 21 and 22, respectively. The nucleotide sequence of the NOV3b variant differs as shown in Table SNP3.

TABLE SNP3

| | Nucleotides | | | Amino Acids | | |
|---|---|---|---|---|---|---|
| Variant | Position | Initial | Modified | Position | Initial | Modified |
| 13375856 | 338 | G | A | 0 | | |
| 13380855 | 397 | T | G | 0 | | |
| 13380857 | 1134 | T | C | 243 | Val | Ala |
| 13375853 | 1362 | G | A | 319 | Arg | His |
| 13380859 | 1376 | A | G | 324 | Thr | Ala |
| 13380860 | 1426 | C | T | 340 | Cys | Cys |
| 13380861 | 1496 | C | T | 0 | | |

NOV4b SNP Data:

NOV4b has eleven SNP variants, whose variant positions for its nucleotide and amino acid sequences are numbered according to SEQ ID NOs: 27 and 28, respectively. The nucleotide sequence of the NOV4b variant differs as shown in Table SNP4.

TABLE SNP4

| | Nucleotides | | | Amino Acids | | |
|---|---|---|---|---|---|---|
| Variant | Position | Initial | Modified | Position | Initial | Modified |
| 13380847 | 73 | C | C | 12 | Arg | Pro |
| 13380848 | 116 | G | A | 26 | Arg | Arg |
| 13380849 | 117 | A | T | 27 | Ile | Phe |
| 13380862 | 200 | G | T | 54 | Lys | Asn |
| 13380863 | 222 | C | T | 62 | Glu | End |
| 13380864 | 243 | C | T | 69 | Glu | End |
| 13380850 | 338 | C | T | 100 | Ile | Ile |
| 13380851 | 438 | C | T | 134 | Ala | Ser |
| 13380865 | 779 | A | T | 247 | Pro | Pro |
| 13380852 | 1023 | C | G | 329 | Pro | Ala |
| 13380853 | 1494 | C | T | 0 | | |

NOV6a SNP Data:

NOV6a has two SNP variants, whose variant positions for its nucleotide and amino acid sequences are numbered according to SEQ ID NOs: 33 and 34, respectively. The nucleotide sequence of the NOV6a variant differs as shown in Table SNP5.

TABLE SNP5

| | Nucleotides | | | Amino Acids | | |
|---|---|---|---|---|---|---|
| Variant | Position | Initial | Modified | Position | Initial | Modified |
| 13380868 | 1646 | T | C | 539 | Val | Ala |
| 13380869 | 2992 | T | C | 988 | Cys | Arg |

NOV11a SNP Data:

NOV11a has one SNP variant, whose variant positions for its nucleotide and amino acid sequences is numbered according to SEQ ID NOs: 47 and 48, respectively. The nucleotide sequence of the NOV11a variant differs as shown in Table SNP6.

TABLE SNP6

| | Nucleotides | | | Amino Acids | | |
|---|---|---|---|---|---|---|
| Variant | Position | Initial | Modified | Position | Initial | Modified |

| | | | | |
|---|---|---|---|---|
| 13380962 | 41 | G | T | 0 |

NOV12a SNP Data:

NOV12a has three SNP variants, whose variant positions for its nucleotide and amino acid sequences are numbered according to SEQ ID NOs: 63 and 64, respectively. The nucleotide sequence of the NOV 12a variant differs as shown in Table SNP7.

TABLE SNF7

| | Nucleotides | | | Amino Acids | | |
|---|---|---|---|---|---|---|
| Variant | Position | Initial | Modified | Position | Initial | Modified |
| 13380902 | 594 | C | T | 193 | Ser | Ser |
| 13380901 | 1392 | A | C | 0 | | |
| 13380900 | 1425 | C | T | 0 | | |

NOV13a SNP Data:

NOV13a has one SNP variant, whose variant positions for its nucleotide and amino acid sequences is numbered according to SEQ ID NOs: 65 and 66, respectively. The nucleotide sequence of the NOV13a variant differs as shown in Table SNP8.

TABLE SNP8

| | Nucleotides | | | Amino Acids | | |
|---|---|---|---|---|---|---|
| Variant | Position | Initial | Modified | Position | Initial | Modified |
| 13380964 | 204 | C | T | 68 | Leu | Leu |

NOV14a SNP Data:

NOV14a has two SNP variants, whose variant positions for its nucleotide and amino acid sequences are numbered according to SEQ ID NOs: 73 and 74, respectively. The nucleotide sequence of the NOV14a variant differs as shown in Table SNP9.

TABLE SNP9

| | Nucleotides | | | Amino Acids | | |
|---|---|---|---|---|---|---|
| Variant | Position | Initial | Modified | Position | Initial | Modified |
| 13380922 | 106 | C | G | 28 | Pro | Pro |
| 13380923 | 760 | A | G | 246 | Pro | Pro |

NOV15a SNP Data:

NOV15a has two SNP variants, whose variant positions for its nucleotide and amino acid sequences are numbered according to SEQ ID NOs: 77 and 78, respectively. The nucleotide sequence of the NOV1Sa variant differs as shown in Table SNP10.

TABLE SNP10

| | Nucleotides | | | Amino Acids | | |
|---|---|---|---|---|---|---|
| Variant | Position | Initial | Modified | Position | Initial | Modified |
| 13380896 | 19 | T | C | 4 | Phe | Leu |
| 13380897 | 258 | G | A | 83 | Pro | Pro |

NOV20a SNP Data:

NOV20a has seven SNP variants, whose variant positions for its nucleotide and amino acid sequences are numbered according to SEQ ID NOs: 107 and 108, respectively. The nucleotide sequence of the NOV20a variant differs as shown in Table SNP11.

TABLE SNP11

| | Nucleotides | | | Amino Acids | | |
|---|---|---|---|---|---|---|
| Variant | Position | Initial | Modified | Position | Initial | Modified |
| 13380969 | 155 | G | A | 0 | | |
| 13380970 | 448 | A | G | 79 | His | Arg |
| 13380971 | 475 | G | C | 88 | Cys | Ser |
| 13380972 | 780 | A | G | 190 | Arg | Gly |
| 13380974 | 890 | A | G | 226 | Arg | Arg |
| 13380975 | 1798 | A | G | 0 | | |
| 13380976 | 2564 | A | G | 0 | | |

V26a SNP Data: V26a has one SNP variant, whose variant positions for its nucleotide and amino ces is numbered according to SEQ ID NOs: 119 and 120, respectively. The sequence of the NOV26a variant differs as shown in Table SNP12.

TABLE SNP12

| | Nucleotides | | | Amino Acids | | |
|---|---|---|---|---|---|---|
| Variant | Position | Initial | Modified | Position | Initial | Modified |
| 13377803 | 98 | C | A | 25 | Met | Ile |

V27a SNP Data:

NOV27a has two SNP variants, whose variant positions for its nucleotide and sequences are numbered according to SEQ ID NOs: 121 and 122, respectively. tide sequence of the NOV27a variant differs as shown in Table SNP13.

TABLE SNP13

| | Nucleotides | | | Amino Acids | | |
|---|---|---|---|---|---|---|
| Variant | Position | Initial | Modified | Position | Initial | Modified |
| 13380980 | 186 | A | G | 22 | Thr | Ala |
| 13380979 | 292 | C | T | 57 | Thr | Ile |

NOV28a SNP Data:

NOV28a has two SNP variants, whose variant positions for its nucleotide and amino acid sequences are numbered according to SEQ ID NOs: 123 and 124, respectively. The nucleotide sequence of the NOV28a variant differs as shown in Table SNP14.

TABLE SNP14

| | Nucleotides | | | Amino Acids | | |
|---|---|---|---|---|---|---|
| Variant | Position | Initial | Modified | Position | Initial | Modified |
| 13380981 | 2192 | G | A | 721 | Arg | Lys |
| 13380982 | 2283 | C | T | 751 | Phe | Phe |

NOV29a SNP Data:

NOV29a has one SNP variant, whose variant positions for its nucleotide and amino acid sequences is numbered according to SEQ ID NOs: 127 and 128, respectively. The nucleotide sequence of the NOV29a variant differs as shown in Table SNP15.

TABLE SNP15

| | Nucleotides | | | Amino Acids | | |
|---|---|---|---|---|---|---|
| Variants | Position | Initial | Modified | Position | Initial | Modified |

| | | | | |
|---|---|---|---|---|
| 13380985 | 46 | T | C | 0 |

NOV31a SNP Data:

NOV31a has one SNP variant, whose variant positions for its nucleotide and amino acid sequences is numbered according to SEQ ID NOs: 133 and 134, respectively. The nucleotide sequence of the NOV31a variant differs as shown in Table SNP16.

TABLE SNP16

| | Nucleotides | | | Amino Acids | | |
|---|---|---|---|---|---|---|
| Variant | Position | Initial | Modified | Position | Initial | Modified |
| 13380984 | 1232 | G | A | 335 | Gly | Ser |

NOV34a SNP Data:

NOV34a has two SNP variants, whose variant positions for its nucleotide and amino acid sequences are numbered according to SEQ ID NOs: 141 and 142, respectively. The nucleotide sequence of the NOV34a variant differs as shown in Table SNP17.

TABLE SNP17

| | Nucleotides | | | Amino Acids | | |
|---|---|---|---|---|---|---|
| Variant | Position | Initial | Modified | Position | Initial | Modified |
| 13380987 | 1145 | G | C | 362 | Arg | Thr |
| 13380988 | 1749 | A | T | 0 | | |

NOV35a SNP Data:

NOV35a has one SNP variant, whose variant positions for its nucleotide and amino acid sequences is numbered according to SEQ ID NOs: 143 and 144, respectively. The nucleotide sequence of the NOV35a variant differs as shown in Table SNP18.

TABLE SNP18

| | Nucleotides | | | Amino Acids | | |
|---|---|---|---|---|---|---|
| Variant | Position | Initial | Modified | Position | Initial | Modified |
| 13380995 | 85 | C | T | 122 | Thr | Ile |

NOV36a SNP Data:

NOV36a has three SNP variants, whose variant positions for its nucleotide and amino acid sequences are numbered according to SEQ ID NOs: 153 and 154, respectively. The nucleotide sequence of the NOV36a variant differs as shown in Table SNP19.

TABLE SNP19

| | Nucleotides | | | Amino Acids | | |
|---|---|---|---|---|---|---|
| Variant | Position | Initial | Modified | Position | Initial | Modified |
| 13380998 | 411 | G | A | 122 | Ser | Asn |
| 13381013 | 492 | T | C | 149 | Leu | Pro |
| 13380999 | 686 | T | C | 214 | Cys | Arg |

NOV37a SNP Data:

NOV37a has one SNP variant, whose variant positions for its nucleotide and amino acid sequences is numbered according to SEQ ID NOs: 155 and 156, respectively. The nucleotide sequence of the NOV37a variant differs as shown in Table SNP20.

TABLE SNP20

| | Nucleotides | | | Amino Acids | | |
|---|---|---|---|---|---|---|
| Variant | Position | Initial | Modified | Position | Initial | Modified |

| | | | | |
|---|---|---|---|---|
| 13381009 | 2077 | C | G | 0 |

NOV38a SNP Data:

NOV38a has one SNP variant, whose variant positions for its nucleotide and amino acid sequences is numbered according to SEQ ID NOs: 157 and 158, respectively. The nucleotide sequence of the NOV38a variant differs as shown in Table SNP21.

TABLE SNP21

| | Nucleotides | | | Amino Acids | | |
|---|---|---|---|---|---|---|
| Variant | Position | Initial | Modified | Position | Initial | Modified |
| 13378369 | 994 | C | T | 330 | Ser | Leu |

NOV40a SNP Data:

NOV40a has one SNP variant, whose variant positions for its nucleotide and amino acid sequences is numbered according to SEQ ID NOs: 167 and 168, respectively. The nucleotide sequence of the NOV40a variant differs as shown in Table SNP22.

TABLE SNP22

| | Nucleotides | | | Amino Acids | | |
|---|---|---|---|---|---|---|
| Variant | Position | Initial | Modified | Position | Initial | Modified |

| | | | | |
|---|---|---|---|---|
| 13381011 | 32 | A | G | 0 |

NOV41a SNP Data:

NOV41a has two SNP variants, whose variant positions for its nucleotide and amino acid sequences are numbered according to SEQ ID NOs: 173 and 174, respectively. The nucleotide sequence of the NOV41a variant differs as shown in Table SNP23.

TABLE SNP23

| | Nucleotides | | | Amino Acids | | |
|---|---|---|---|---|---|---|
| Variant | Position | Initial | Modified | Position | Initial | Modified |
| 13380997 | 247 | A | G | 55 | Asn | Asp |
| 13380996 | 417 | A | G | 111 | Lys | Lys |

NOV43a SNP Data:

NOV43a has eight SNP variants, whose variant positions for its nucleotide and amino acid sequences are numbered according to SEQ ID NOs: 181 and 182, respectively. The nucleotide sequence of the NOV43a variant differs as shown in Table SNP24.

TABLE SNP24

| | Nucleotides | | | Amino Acids | | |
|---|---|---|---|---|---|---|
| Variant | Position | Initial | Modified | Position | Initial | Modified |
| 13381140 | 184 | G | A | 61 | Asp | Asn |
| 13381141 | 337 | T | C | 112 | Phe | Leu |
| 13381158 | 729 | G | T | 242 | Met | Ile |
| 13381157 | 748 | A | G | 249 | Ser | Gly |
| 13381156 | 934 | T | C | 311 | Phe | Leu |
| 13381142 | 1916 | A | G | 0 | | |
| 13381143 | 2123 | T | A | 0 | | |
| 13381148 | 2260 | G | C | 0 | | |

NOV44a SNP Data:

NOV44a has one SNP variant, whose variant positions for its nucleotide and amino acid sequences is numbered according to SEQ ID NOs: 183 and 184, respectively. The nucleotide sequence of the NOV44a variant differs as shown in Table SNP25.

TABLE SNP25

| | Nucleotides | | | Amino Acids | | |
|---|---|---|---|---|---|---|
| Variant | Position | Initial | Modified | Position | Initial | Modified |

| | | | | |
|---|---|---|---|---|
| 13381168 | 1096 | C | T | 0 |

NOV45a SNP Data:

NOV45a has two SNP variants, whose variant positions for its nucleotide and amino acid sequences are numbered according to SEQ ID NOs: 185 and 186, respectively. The nucleotide sequence of the NOV45a variant differs as shown in Table SNP26.

TABLE SNP26

| | Nucleotides | | | Amino Acids | | |
|---|---|---|---|---|---|---|
| Variant | Position | Initial | Modified | Position | Initial | Modified |
| 13381163 | 1269 | T | C | 399 | Cys | Arg |
| 13381162 | 1418 | C | T | 0 | | |

NOV46a SNP Data:

NOV46a has one SNP variant, whose variant positions for its nucleotide and amino acid sequences is numbered according to SEQ ID NOs: 187 and 188, respectively. The nucleotide sequence of the NOV46a variant differs as shown in Table SNP27.

TABLE SNP27

| | Nucleotides | | | Amino Acids | | |
|---|---|---|---|---|---|---|
| Variant | Position | Initial | Modified | Position | Initial | Modified |
| 13381020 | 820 | T | C | 267 | Phe | Phe |

NOV48b SNP Data:

NOV48b has five SNP variants, whose variant positions for its nucleotide and amino acid sequences are numbered according to SEQ ID NOs: 193 and 194, respectively. The nucleotide sequence of the NOV48b variant differs as shown in Table SNP28.

TABLE SNP28

| | Nucleotides | | | Amino Acids | | |
|---|---|---|---|---|---|---|
| Variant | Position | Initial | Modified | Position | Initial | Modified |
| 13375777 | 107 | A | G | 14 | His | Arg |
| 13376584 | 116 | G | A | 17 | Ser | Asn |
| 13381146 | 448 | T | C | 128 | Cys | Arg |
| 13378857 | 1282 | G | A | 406 | Gly | Ser |
| 13376583 | 1297 | C | T | 411 | Pro | Ser |

NOV49a SNP Data:

NOV49a has twenty-one SNP variants, whose variant positions for its nucleotide and amino acid sequences are numbered according to SEQ ID NOs: 195 and 196, repectively. The nucleotide sequence of the NOV49a variant differs as shown in Table SNP29.

TABLE SNP29

| | Nucleotides | | | Amino Acids | | |
|---|---|---|---|---|---|---|
| Variant | Position | Initial | Modified | Position | Initial | Modified |
| 13379126 | 186 | C | T | 17 | Ala | Val |
| 13375663 | 212 | C | G | 26 | Leu | Val |
| 13375662 | 213 | T | C | 26 | Leu | Pro |
| 13379016 | 293 | A | G | 53 | Ser | Gly |
| 13378698 | 388 | C | T | 84 | Phe | Phe |
| 13381282 | 401 | C | T | 89 | Gln | End |
| 13381193 | 556 | A | C | 140 | Thr | Thr |
| 13381194 | 577 | G | A | 147 | Gly | Gly |
| 13381283 | 631 | A | G | 165 | Lys | Lys |
| 13378699 | 840 | G | A | 235 | Ser | Asn |
| 13378106 | 909 | A | G | 258 | Asp | Gly |
| 13381284 | 924 | A | G | 263 | Lys | Arg |
| 13377887 | 954 | A | G | 273 | Glu | Gly |
| 13381285 | 967 | C | T | 277 | Gly | Gly |
| 13381286 | 1009 | A | G | 291 | Thr | Thr |
| 13377889 | 1083 | A | G | 316 | Gln | Arg |
| 13381287 | 1107 | A | C | 324 | Glu | Gly |
| 13377890 | 1113 | T | C | 326 | Val | Ala |
| 13377891 | 1137 | A | C | 334 | Gln | Pro |
| 13381288 | 1196 | C | G | 0 | | |
| 13381289 | 1202 | A | G | 0 | | |

NOV50b SNP Data:

NOV50b has three SNP variants, whose variant positions for its nucleotide and amino acid sequences are numbered according to SEQ ID NOs: 219 and 220, respectively. The nucleotide sequence of the NOV50b variant differs as shown in Table SNP30.

TABLE SNP23

| | Nucleotides | | | Amino Acids | | |
|---|---|---|---|---|---|---|
| Variant | Position | Initial | Modified | Position | Initial | Modified |
| 13381192 | 216 | G | A | 48 | Glu | Glu |
| 13381177 | 602 | G | T | 177 | Arg | Leu |
| 13381190 | 698 | C | T | 0 | | |

NOV52b SNP Data:

NOV52b has eight SNP variants, whose variant positions for its nucleotide and amino acid sequences are numbered according to SEQ ID NOs: 229 and 230, respectively. The nucleotide sequence of the NOV52b variant differs as shown in Table SNP31.

TABLE SNP31

| | Nucleotides | | | Amino Acids | | |
|---|---|---|---|---|---|---|
| Variant | Position | Initial | Modified | Position | Initial | Modified |
| 13381176 | 215 | A | G | 43 | Glu | Glu |
| 13376180 | 320 | C | T | 78 | Tyr | Tyr |
| 13376179 | 397 | A | G | 104 | Gln | Arg |
| 13381171 | 519 | T | C | 145 | Ser | Pro |
| 13381174 | 629 | C | T | 181 | Ile | Ile |
| 13381173 | 1173 | C | A | 363 | Gln | Lys |
| 13381172 | 1174 | A | C | 363 | Gln | Pro |
| 13381169 | 1402 | A | G | 0 | | |

NOV53c SNP Data:

NOV53c has two SNP variants, whose variant positions for its nucleotide and amino acid sequences are numbered according to SEQ ID NOs: 237 and 238, respectively. The nucleotide sequence of the NOV53c variant differs as shown in Table SNP32.

TABLE SNP32

| | Nucleotides | | | Amino Acids | | |
|---|---|---|---|---|---|---|
| Variant | Position | Initial | Modified | Position | Initial | Modified |
| 13380578 | 424 | C | T | 136 | Asp | Asp |
| 13380577 | 869 | A | G | 285 | Thr | Ala |

NOV55a SNP Data:

NOV55a has thirteen SNP variants, whose variant positions for its nucleotide and amino acid sequences are numbered according to SEQ ID NOs: 245 and 246, respectively. The nucleotide sequence of the NOV55a variant differs as shown in Table SNP33.

TABLE SNP33.

| | Nucleotides | | | Amino Acids | | |
|---|---|---|---|---|---|---|
| Variant | Position | Initial | Modified | Position | Initial | Modified |
| 13375283 | 272 | C | T | 0 | | |
| 13375284 | 281 | T | C | 0 | | |
| 13377920 | 1226 | T | C | 203 | Ser | Pro |
| 13377921 | 1447 | C | T | 276 | Tyr | Tyr |
| 13377922 | 1765 | C | T | 382 | Gly | Gly |
| 13377907 | 2021 | A | G | 468 | Thr | Ala |
| 13377908 | 2074 | T | C | 485 | Tyr | Tyr |
| 13375287 | 2153 | G | C | 512 | Val | Leu |
| 13375288 | 2157 | C | T | 513 | Pro | Leu |
| 13375289 | 2160 | C | T | 514 | Thr | lie |
| 13375290 | 2329 | G | A | 0 | | |
| 13377903 | 2417 | A | G | 0 | | |
| 13377904 | 2559 | C | T | 0 | | |

### Example E: Potential Role(s) of CG96736-01 in Obesity and/or Diabetes

The NOV55a gene (CG96736-01) is a Na+-dependent neutral amino acid transporter that exhibits high affinity electroneutral uptake of neutral amino acids such as L-alanine, L-serine, L-threonine, L-cysteine and L-glutamine. This transporter prefers neutral amino acids without bulky or branched side chains. It is localized to the plasma membrane and has eight putative transmembrane segments. It appears to be a Type IIIa membrane protein with an N-terminal cytoplasmic tail and a C-terminal extracellular segment. In this respect, the expression patter and its function in nutral amino acid uptake is an indication of a role for NOV55a in obesity and/or diabetes.

Obesity and Diabetes are major public health concerns in the developed and developing world. It is estimated that over half of the adult US population is overweight with a body mass index (BMI) greater than the upper limit of normal (25) where the BMI is defined as the weight (Kg)/[height (m)]². A common consequence of being overweight is hyperlipidemia and the development of insulin resistance. This is followed by the development of hyperglycemia—a hallmark of Type II diabetes. Left untreated, the hyperglycemia leads to microvascular disease and end organ damage that includes retinopathy, renal disease, cardiac disease, peripheral neuropathy and peripheral vascular compromise. Currently, over 16 million adults in the US are affected and the condition has now become rampant among school-age children as a consequence of the epidemic of obesity in that age group.

Several cellular, animal and clinical studies were performed to elucidate the genetic contribution to the etiology and pathogenesis of these conditions in a variety of physiologic, pharmacologic or native states. These studies utilized the core technologies at CuraGen Corporation to look at differential gene expression, protein-protein interactions, large-scale sequencing of expressed genes and the association of genetic variations such as, but not limited to, single nucleotide polymorphisms (SNPs) or splice variants in and between biological samples from experimental and control groups. The goal of such studies is to identify potential avenues for therapeutic intervention in order to prevent, treat the consequences or cure the conditions.

In order to treat diseases, pathologies and other abnormal states or conditions in which a mammalian organism has been diagnosed as being, or as being at risk for becoming, other than in a normal state or condition, it is important to identify new therapeutic agents. Such a procedure includes at least the steps of identifying a target component within an affected tissue or organ, and identifying a candidate therapeutic agent that modulates the functional attributes of the target. The target component may be any biological macromolecule implicated in the disease or pathology. Commonly the target is a polypeptide or protein with specific functional attributes. Other classes of macromolecule may be a nucleic acid, a polysaccharide, a lipid such as a complex lipid or a glycolipid; in addition a target may be a sub-cellular structure or extra-cellular structure that is comprised of more than one of these classes of macromolecule. Once such a target has been identified, it may be employed in a screening assay in order to identify favorable candidate therapeutic agents from among a large population of substances or compounds.

In many cases the objective of such screening assays is to identify small molecule candidates; this is commonly approached by the use of combinatorial methodologies to develop the population of substances to be tested. The implementation of high throughput screening methodologies is advantageous when working with large, combinatorial libraries of compounds.

In an important aspect, the present invention provides a method of identifying a candidate therapeutic agent for treating a disease, pathology, or an abnormal state or condition using a target entity having a specific association with the disease. This method includes:

(a) identification of a target biopolymer associated with the disease, pathology, or abnormal state or condition;

(b) contacting the biopolymer with at least one chemical compound; and

(c) identifying a compound that binds to the biopolymer as a candidate therapeutic agent.

In important embodiments of this method, the chemical compound is a member of a combinatorial library of compounds; the contacting in step (b) is conducted on one or more replicate samples of the biopolymer; and the replicate sample is contacted with at least one member of the combinatorial library. In additional embodiments of this method, the biopolymer is included within a cell and is functionally expressed therein. In still a further advantageous embodiment, the binding of the compound modulates the function of the biopolymer, and it is the modulation that provides the identification that the compound is a potential therapeutic agent. In yet further significant embodiments of this method, the target biopolymer is a polypeptide.

In a second aspect of the invention, a method for identifying a pharmaceutical agent for treating a disease, pathology, or an abnormal state or condition is provided. The second method includes the steps of:

(a) identifying a candidate therapeutic agent for treating said disease, pathology, or abnormal state or condition by the method described in the preceding paragraph;

(b) contacting a biological sample associated with the disease, pathology, or abnormal state or condition with the candidate therapeutic agent;

(c) determining whether the candidate induces an effect on the biological sample associated with a therapeutic response therein; and

(d) identifying a candidate exerting such an effect as a pharmaceutical agent.

In significant embodiments of the second method, the biological sample includes a cell, a tissue or organ, or is a nonhuman mammal.

A gene fragment of the mouse Neutral Amino Acid Transporter B was initially found to be up-regulated by 6 fold in the adipose tissue of obese mice (AKR) relative to non-obese mice (C57BL/6J) using CuraGen's GeneCalling™ method of differential gene expression. Two differentially expressed mouse gene fragments migrating, at approximately 138 and 347 nucleotides in length (Tables MOU-3A and MOU-3B for NOV55c (SEQ ID NO: 438), and Tables MOU-3C and MOU-3D for NOV55d (SEQ ID NO: 439) respectively—vertical line) were definitively identified as a component of the Mouse Neutral Amino Acid Transporter B cDNA (in the graphs, the abscissa is measured in lengths of nucleotides and the ordinate is measured as signal response). The method of competitive PCR was used for conformation of the gene assessment. The electropherogramatic peaks corresponding to the gene fragment of the mouse Neutral Amino Acid Transporter B are ablated when a gene-specific primer competes with primers in the linker-adaptors during the PCR amplification. The peaks at 138 nt length are ablated in the sample from both the obese and non-obese mice.

The direct sequences of the 138.4 and 346.7 nucleotide-long gene fragments and the gene-specific primers used for competitive PCR are indicated on the cDNA sequence of the Mouse Neutral Amino Acid Transporter B are shown below in bold. The gene-specific primers at the 5′ and 3′ ends of the fragment are in italics.

Competitive PCR Primer for the Mouse Neutral Amino Acid Transporter B (peak at 138.4).

TABLE MOU-1

NOV55c Gene Sequence
(fragment from 564 to 700 in bold. band size: 137)

(SEQ ID NO:438)

| | | |
|---|---|---|
| 83 | CCAGAGAGGA CCAGAGTGCG AAAGCAGGTG GTTGCTGCGG TTCCCGTGAC CGGGTGCGCC | 143 |

| | |
|---|---|
| GCTGCATTCG CGCCAACCTG CTGGTGCTGC TCACGGTGGC TGCGGTGGTG GCTGGCGTGG | 203 |
| GGCTGGGGCT GGGGGTCTCG GCGGCGGGCG GTGCTGACGC CCTGGGTCCC GCGCGCTTGA | 263 |
| CCGCTTTCGC CTTCCCGGGA GAGCTGCTGC TGCGTCTGCT GAAGATGATC ATCCTGCCCC | 323 |
| TCGTGGTGTG CAGCCTGATC GGAGGTGCAG CCAGCTTGGA CCCTAGCGCG CTCGGTCGTG | 383 |
| TGGGCGCCTG GGCGCTGCTC TTTTTCCTGG TCACCACACT GCTCGCGTCG GCGCTCGGCG | 443 |
| TGGGTTTGGC CCTGGCGCTG AAGCCCGGCG CCGCCGTTAC CGCCATCACC TCCATCAACG | 503 |
| ACTCTGTTGT AGACCCCTGT GCCCGCAGTG CACCAACCAA AGAGGTGCTG GATTCCTTTC | 563 |
| TAGATCTCGT CAGGAATATT TTCCCCTCCA ATCTGGTGTC TGCTGCCTTC CGCTCTTTTG | 623 |
| CTACCTCATA TGAACCCAAA GACAACTCAT GTAAAATACC GCAATCCTGT ATCCAGCGGG | 683 |
| AGATCAATTC AACCATGGTC CAGCTTCTCT GTGAGGTGGA GCGAATGAAC ATCCTGGGCC | 743 |
| TGGTGGTCTT CGCTATCGTC TTTGGTGTGG CTCTGCGGAA GCTGGGGCCC GAGGGTGAGC | 803 |
| TGCTCATTCG TTTCTTCAAC TCCTTCAATG ATGCCACCAT GGTCCTGGTC TCCTGGATTA | 863 |
| TGTGGTACGC ACCCGTTGGA ATCCTGTTCC TGGTGGCCAG CAAGATTGTG GAGATGAAAG | 923 |
| ACGTCCGCCA GCTCTTCATC AGCCTCGGCA AATACATTCT GTGCTGCCTG CTGGGCCACG | 983 |
| CCATCCACGG GCTCCTGGTT CTGCCTCTCA TCTACTTCCT CTTCACCCGC AAAAATCCCT | 1043 |
| ATCGATTCCT GTGGGGCATC ATGACACCCC TGGCCACTGC TTTCGGGACC TCTTCTAGCT | 1103 |
| CTGCCACCTT GCCTCTGATG ATGAAGTGTG TAGAGGAGAA GAATGGTGTG GCCAAACACA | 1163 |
| TCAGCCGGTT CATCCTAC | |

(gene length is 1668, only region from 83 to 1180 shown)

Competitive PCR Primer for the Mouse Neutral Amino Acid Transporter B (peak at 346.7). The gene-specific primers at the 5′ and 3′ ends of the fragment are in italics.

TABLE MOU-2

NOV55d Gene Sequence
(fragment from 1 to 347 in italics, band size: 347)

(SEQ ID NO:439)

| | |
|---|---|
| GGAPCCCTGC CGCACCGACA CTGGATGCTG TGGCTGTGAC CCTGGGGAAG AGAAGAGCGG | 61 |
| AGATGGCAGA ATCATGGGGG CGGGGCCTCC TGCCACAGCC CCTGGCACTC ACAGGATGGT | 121 |
| GATGATCTTC ACGAAGTCCA GCGACACCCC GTTTAGTTGT GCGATGAACA CTGCCGCCAC | 181 |
| ACACTGGAAC AGCGCCGCCC CGTCCATGTT GACCGTCGCG CCGATGGGTA GGATCAACCG | 241 |
| GCTGATGTGT TTGGCCACAC CATTCTTCTC CTCTACACAC TTCATCATCA GAGGCAAGGT | 301 |
| GGCAGAGCTA GAAGAGGTCC CGAAAGCAGT GGCCAGGGGT GTCATGA | |

(gene length is 347, only region from 1 to 347 shown)

Nucleic acid and amino acid sequences for NOV55a and NOV55b are disclosed in Table 55a, SNPs for NOV55a and NOV55b are disclosed in Table SNP33 and quantitative expression of these genes is shown in Tables AUA-AUK in Example D.

Tables MOU-3A and MOU-3B show differentially expressed mouse neutral amino acid transporter B gene fragment, NOV55c, and Tables MOU-3C and MOU-3D shows differentially expressed mouse neutral amino acid transporter B gene fragment, NOV55d.

[image]

[image]

[image]

### OTHER EMBODIMENTS

Although particular embodiments have been disclosed herein in detail, this has been done by way of example for purposes of illustration only, and is not intended to be limiting with respect to the scope of the appended claims, which follow. In particular, it is contemplated by the inventors that various substitutions, alterations, and modifications may be made to the invention without departing from the spirit and scope of the invention as defined by the claims. The choice of nucleic acid starting material, clone of interest, or library type is believed to be a matter of routine for a person of ordinary skill in the art with knowledge of the embodiments described herein. Other aspects, advantages, and modifications considered to be within the scope of the following claims. The claims presented are representative of the inventions disclosed herein. Other, unclaimed inventions are also contemplated. Applicants reserve the right to pursue such inventions in later claims.

What is claimed is:

1. An isolated polypeptide comprising the mature form of an amino acid sequenced selected from the group consisting of SEQ ID NO: 2n, wherein n is an integer between 1 and 124.

2. An isolated polypeptide comprising an amino acid sequence selected from the group consisting of SEQ ID NO: 2n, wherein n is an integer between 1 and 124.

3. An isolated polypeptide comprising an amino acid sequence which is at least 95% identical to an amino acid sequence selected from the group consisting of SEQ ID NO: 2n, wherein n is an integer between 1 and 124.

4. An isolated polypeptide, wherein the polypeptide comprises an amino acid sequence comprising one or more conservative substitutions in the amino acid sequence selected from the group consisting of SEQ ID NO: 2n, wherein n is an integer between 1 and 124.

5. The polypeptide of claim 1 wherein said polypeptide is naturally occurring.

6. A composition comprising the polypeptide of claim 1 and a carrier.

7. A kit comprising, in one or more containers, the composition of claim 6.

8. The use of a therapeutic in the manufacture of a medicament for treating a syndrome associated with a human disease, the disease selected from a pathology associated with the polypeptide of claim l, wherein the therapeutic comprises the polypeptide of claim 1.

9. A method for determining the presence or amount of the polypeptide of claim 1 in a sample, the method comprising:
(a) providing said sample;
(b) introducing said sample to an antibody that binds immunospecifically to the polypeptide; and
(c) determining the presence or amount of antibody bound to said polypeptide, thereby determining the presence or amount of polypeptide in said sample.

10. A method for determining the presence of or predisposition to a disease associated with altered levels of expression of the polypeptide of claim 1 in a first mammalian subject, the method comprising:
a) measuring the level of expression of the polypeptide in a sample from the first mammalian subject; and
b) comparing the expression of said polypeptide in the sample of step (a) to the expression of the polypeptide present in a control sample from a second mammalian subject known not to have, or not to be predisposed to, said disease,
wherein an alteration in the level of expression of the polypeptide in the first subject as compared to the control sample indicates the presence of or predisposition to said disease.

11. A method of identifying an agent that binds to the polypeptide of claim 1, the method comprising:
(a) introducing said polypeptide to said agent; and
(b) determining whether said agent binds to said polypeptide.

12. The method of claim 11 wherein the agent is a cellular receptor or a downstream effector.

13. A method for identifying a potential therapeutic agent for use in treatment of a pathology, wherein the pathology is related to aberrant expression or aberrant physiological interactions of the polypeptide of claim 1, the method comprising:
(a) providing a cell expressing the polypeptide of claim 1 and having a property or function ascribable to the polypeptide;
(b) contacting the cell with a composition comprising a candidate substance; and
(c) determining whether the substance alters the property or function ascribable to the polypeptide;
whereby, if an alteration observed in the presence of the substance is not observed when the cell is contacted with a composition in the absence of the substance, the substance is identified as a potential therapeutic agent.

14. A method for screening for a modulator of activity of or of latency or predisposition to a pathology associated with the polypeptide of claim 1, said method comprising:
(a) administering a test compound to a test animal at increased risk for a pathology associated with the polypeptide of claim 1, wherein said test animal recombinantly expresses the polypeptide of claim 1;
(b) measuring the activity of said polypeptide in said test animal after administering the compound of step (a); and
(c) comparing the activity of said polypeptide in said test animal with the activity of said polypeptide in a control animal not administered said polypeptide, wherein a change in the activity of said polypeptide in said test animal relative to said control animal indicates the test compound is a modulator activity of or latency or predisposition to, a pathology associated with the polypeptide of claim 1.

15. The method of claim 14, wherein said test animal is a recombinant test animal that expresses a test protein transgene or expresses said transgene under the control of a promoter at an increased level relative to a wild-type test animal, and wherein said promoter is not the native gene promoter of said transgene.

16. A method for modulating the activity of the polypeptide of claim 1, the method comprising contacting a cell sample expressing the polypeptide of claim 1 with a compound that binds to said polypeptide in an amount sufficient to modulate the activity of the polypeptide.

17. A method of treating or preventing a pathology associated with the polypeptide of claim 1, the method comprising administering the polypeptide of claim 1 to a subject in which such treatment or prevention is desired in an amount sufficient to treat or prevent the pathology in the subject.

18. The method of claim 17, wherein the subject is a human.

19. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising the amino acid sequence selected from the group consisting of SEQ ID NO: 2n, wherein n is an integer between 1 and 124 or a biologically active fragment thereof.

20. An isolated nucleic acid molecule comprising a nucleic acid sequence selected from the group consisting of SEQ ID NO: 2n−1, wherein n is an integer between 1 and 124.

21. The nucleic acid molecule of claim 20, wherein the nucleic acid molecule is naturally occurring.

22. A nucleic acid molecule, wherein the nucleic acid molecule differs by a single nucleotide from a nucleic acid sequence selected from the group consisting of SEQ ID NO: 2n−1, wherein n is an integer between 1 and 124.

23. An isolated nucleic acid molecule encoding the mature form of a polypeptide having an amino acid sequence selected from the group consisting of SEQ ID NO: 2n, wherein n is an integer between 1 and 124.

24. An isolated nucleic acid molecule comprising a nucleic acid selected from the group consisting of 2n−1, wherein n is an integer between 1 and 124.

25. The nucleic acid molecule of claim 20, wherein said nucleic acid molecule hybridizes under stringent conditions to the nucleotide sequence selected from the group consisting of SEQ ID NO: 2n−1, wherein n is an integer between 1 and 124, or a complement of said nucleotide sequence.

26. A vector comprising the nucleic acid molecule of claim 20.

27. The vector of claim 26, further comprising a promoter operably linked to said nucleic acid molecule.

28. A cell comprising the vector of claim 26.

29. An antibody that immunospecifically binds to the polypeptide of claim 1.

30. The antibody of claim 29, wherein the antibody is a monoclonal antibody.

31. The antibody of claim 29, wherein the antibody is a humanized antibody.

32. A method for determining the presence or amount of the nucleic acid molecule of claim 20 in a sample, the method comprising:
(a) providing said sample;
(b) introducing said sample to a probe that binds to said nucleic acid molecule;
and
(c) determining the presence or amount of said probe bound to said nucleic acid molecule,
thereby determining the presence or amount of the nucleic acid molecule in said sample.

33. The method of claim 32 wherein presence or amount of the nucleic acid molecule is used as a marker for cell or tissue type.

34. The method of claim 33 wherein the cell or tissue type is cancerous.

35. A method for determining the presence of or predisposition to a disease associated with altered levels of expression of the nucleic acid molecule of claim 20 in a first mammalian subject, the method comprising:
a) measuring the level of expression of the nucleic acid in a sample from the first mammalian subject; and
b) comparing the level of expression of said nucleic acid in the sample of step (a) to the level of expression of the nucleic acid present in a control sample from a second mammalian subject known not to have or not be predisposed to, the disease;
wherein an alteration in the level of expression of the nucleic acid in the first subject as compared to the control sample indicates the presence of or predisposition to the disease.

36. A method of producing the polypeptide of claim 1, the method comprising culturing a cell under conditions that lead to expression of the polypeptide, wherein said cell comprises a vector comprising an isolated nucleic acid molecule comprising a nucleic acid sequence selected from the group consisting of SEQ ID NO: 2n−1, wherein n is an integer between 1 and 124.

37. The method of claim 36 wherein the cell is a bacterial cell.

38. The method of claim 36 wherein the cell is an insect cell.

39. The method of claim 36 wherein the cell is a yeast cell.

40. The method of claim 36 wherein the cell is a mammalian cell.

41. A method of producing the polypeptide of claim 2, the method comprising culturing a cell under conditions that lead to expression of the polypeptide, wherein said cell comprises a vector comprising an isolated nucleic acid molecule comprising a nucleic acid sequence selected from the group consisting of SEQ ID NO: 2n−1, wherein n is an integer between 1 and 124.

42. The method of claim 41 wherein the cell is a bacterial cell.

43. The method of claim 41 wherein the cell is an insect cell.

44. The method of claim 41 wherein the cell is a yeast cell.

45. The method of claim 41 wherein the cell is a mammalian cell.
